(19) United States
(12) Patent Application Publication
(10) Pub. No.: US 2025/0302995 A1
(43) Pub. Date: Oct. 2, 2025

(54) OLIGONUCLEOTIDE COMPOSITIONS AND METHODS THEREOF

(71) Applicants: Chikdu Shakti Shivalila, Cambridge, MA (US); Genliang Lu, Cambridge, MA (US); Ian Chandler Harding, Cambridge, MA (US); Jack David Godfrey, Cambridge, MA (US); Tom Liantang Pu, Cambridge, MA (US); Stephany Michelle Standley, Cambridge, MA (US); Pachamuthu Kandasamy, Cambridge, MA (US); Chandra Vargeese, Cambridge, MA (US); WAVE LIFE SCIENCES LTD., Singapore (SG)

(72) Inventors: Chikdu Shakti Shivalila, Woburn, MA (US); Genliang Lu, Winchester, MA (US); Ian Chandler Harding, Waltham, MA (US); Jack David Godfrey, Arlington, MA (US); Tom Liantang Pu, Boston, MA (US); Stephany Michelle Standley, Wakefield, MA (US); Pachamuthu Kandasamy, Lexington, MA (US); Chandra Vargeese, Schwenksville, PA (US)

(21) Appl. No.: 18/864,860

(22) PCT Filed: May 12, 2023
(86) PCT No.: PCT/US23/22142

§ 371 Date: Nov. 11, 2024

Related U.S. Application Data
(60) Provisional application No. 63/341,407, filed on May 12, 2022.

(51) Int. Cl. A61K 48/00; A61K 31/713; C07H 21/02; C12N 9/78; C12N 15/11

(52) U.S. Cl. CPC A61K 48/005; A61K 31/713; C07H 21/02; C12N 9/78; C12N 15/111; C12N 2310/314; C12N 2310/322; C12Y 305/04004

(57) ABSTRACT

Among other things, the present disclosure provides oligonucleotides, compositions and methods thereof that are useful for adenosine modification. In some embodiments, the present disclosure provides methods for treating various conditions, disorders or diseases that can benefit from adenosine modification.

### CROSS-REFERENCE TO RELATED APPLICATIONS

This application claims priority to U.S. Provisional Application Ser. No. 63/341,407 filed May 12, 2022, the entirety of which is incorporated herein by reference.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

When utilized for various applications, an oligonucleotide is typically prepared to have a base sequence that is complementary to a single stretch of consecutive nucleobases in a target nucleic acid. For example, for target adenosine editing by ADAR, the base sequence of an oligonucleotide is typically complementary to the base sequence of a portion of a target nucleic acid that includes a target adenosine.

Surprisingly, the present disclosure demonstrates that, among other things, an oligonucleotide can have two or more domains, each of which independently has a base sequence that is complementary to that of a portion of a target nucleic acid, wherein two or more portions of the target nucleic acid are independently separated by a gap. As demonstrated herein, such oligonucleotides can surprisingly provide various biological activities and are useful for various applications, e.g., target adenosine editing by ADAR. In some embodiments, the present disclosure provides an oligonucleotide comprising:
a first domain; and
a second domain,
   wherein:
the base sequence of the first domain is complementary to a first portion of the base sequence of a target nucleic acid;
the base sequence of the second domain is complementary to a second portion of the base sequence of a target nucleic acid; and
the first portion and the second portion of the base sequence of the target nucleic acid are separated by a gap.

Provided technologies, e.g., oligonucleotides, compositions, methods, etc., can provide various advantages. For example, in some embodiments, provided technologies provide greatly expanded options with respect to base sequence of oligonucleotides. For example, for target adenosine editing, base sequence of an oligonucleotide does not have to be complimentary to a portion of a target nucleic acid within a certain range from a target adenosine (e.g., about 25-50 or more consecutive nucleosides including a target adenosine): part of an oligonucleotide, e.g., a first portion, may have a base sequence that is complementary to a distal portion of a target nucleic acid from a target adenosine. Alternatively or additionally, a shorter portion around a target adenosine may be targeted. In some embodiments, such shorter portion avoids disruption of one or more functional elements near to a target adenosine (e.g., within about 5, 10, 15, 20, 30, 40, or 50 nucleosides from a target adenosine), e.g., motifs critical for splicing, regulation, etc. Alternatively or additionally, provided technologies, by providing options to utilize sequences complimentary to portions away from target adenosines, can improve various properties and/or activities, e.g., stability, cell uptake, non-specific interaction, editing efficiency, etc. Alternatively or additionally, provided technologies enable or improve editing of target adenosines in complex structures, e.g., complex secondary RNA structures. Alternatively or additionally, provided technologies can provide different on/off rates. In some embodiments, base sequence of a reference oligonucleotide for comparison is, or comprises a sequence that is, complementary to the base sequence of one and only one portion of a target nucleic acid, which portion includes a target adenosine and is about 25-100, 30-80, 30-70, 30-60, 30-50, 25-50 or 25-40 nucleobases in length.

In some embodiments, the present disclosure provides designed oligonucleotides and compositions thereof which oligonucleotides comprise modifications (e.g., modifications to nucleobases sugars, and/or internucleotidic linkages, and patterns thereof) as described herein. In some embodiments, technologies (compounds (e.g., oligonucleotides), compositions, methods, etc.) of the present disclosure (e.g., oligonucleotides, oligonucleotide compositions, methods, etc.) are particularly useful for editing nucleic acids, e.g., site-directed editing in nucleic acids (e.g., editing of target adenosine). In some embodiments, provided technologies can significantly improve efficiency of nucleic acid editing, e.g., modification of one or more A residues, such as conversion of A to I. In some embodiments, the present disclosure provides technologies for editing (e.g., for modifying an A residue, e.g., converting an A to I) in an RNA. In some embodiments, the present disclosure provides technologies for editing (e.g., for modifying an A residue, e.g., converting an A to an I) in a transcript, e.g., mRNA. Among other things, provided technologies provide the benefits of utilization of endogenous proteins such as ADAR (Adenosine Deaminases Acting on RNA) proteins (e.g., ADAR1 and/or ADAR2), for editing nucleic acids, e.g., for modifying an A (e.g., as a result of G to A mutation). Those skilled in the art will appreciates that such utilization of endogenous proteins can avoid a number of challenges and/or provide various benefits compared to those technologies that require the delivery of exogenous components (e.g., proteins (e.g., those engineered to bind to oligonucleotides (and/or duplexes thereof with target nucleic acids) to provide desired activities), nucleic acids encoding proteins, viruses, etc.).

Particularly, in some embodiments, oligonucleotides of provided technologies comprise useful sugar modifications and/or patterns thereof (e.g., presence and/or absence of certain modifications), nucleobase modifications and/or patterns thereof (e.g., presence and/or absence of certain modifications), internucleotidic linkages modifications and/or stereochemistry and/or patterns thereof [e.g., types, modifications, and/or configuration (Rp or Sp) of chiral linkage phosphorus, etc.], etc., which, when combined with one or more other structural elements described herein (e.g., additional chemical moieties) can provide high activities and/or various desired properties, e.g., high efficiency of nucleic acid editing, high selectivity, high stability, high cellular uptake, low immune stimulation, low toxicity, improved distribution, improved affinity, etc. In some embodiments, provided oligonucleotides provide high stability, e.g., when compared to oligonucleotides having a high percentage of natural RNA sugars utilized for adenosine editing. In some embodiments, provided oligonucleotides provide high activities, e.g., adenosine editing activity. In some embodiments, provided oligonucleotides provide high selectivity, for example, in some embodiments, provided oligonucleotides provide selective modification of a target adenosine in a target nucleic acid over other adenosine in the same target nucleic acid (e.g., 2, 3, 4, 5, 6, 7, 8, 9, 10, 11, 12, 13, 14, 15, 16, 17, 18, 19, 20 fold or more modification at the target adenosine than another adenosine, or all other adenosine, in a target nucleic acid).

Among other things, the present disclosure provides designed oligonucleotides and compositions of improved properties and/or activities compared to reference oligonucleotides and compositions (e.g., those described herein or reported in the art). For example, in some embodiments, provided oligonucleotide and compositions can provide improved stability, pharmacokinetic properties, pharmacodynamic properties and/or improved activities (e.g., for A-to-I editing). Various designed oligonucleotides and compositions are described herein. For example, in some embodiments, the present disclosure provides oligonucleotides and compositions thereof, including chirally controlled oligonucleotide compositions thereof, wherein the oligonucleotides comprise several (e.g., 1, 2, 3, 4, or 5 or more; in some embodiments, 3 or more) nucleosides independently comprising sugar modifications (e.g., 2′-OR modifications wherein R is optionally substituted C₁₋₆ alkyl (e.g., 2′-OMe, 2′-MOE, etc.,), bicyclic sugars (e.g., LNA sugars, cEt sugars, etc.)) at their 5′- and 3′-ends. In some embodiments, the first several (e.g., 1, 2, 3, 4, or 5 or more; in some embodiments, 3 or more) nucleosides and/or the last several (e.g., 1, 2, 3, 4, or 5 or more; in some embodiments, 3 or more) nucleosides independently comprise sugar modifications. In some embodiments, the first 3 or more and the last 3 or more nucleosides independently comprise sugar modifications. In some embodiments, one or more internucleotidic linkages bonded to such nucleosides are non-negatively charged internucleotidic linkage such as phosphoryl guanidine internucleotidic linkages like n001. In some embodiments, both the first and the last internucleotidic linkages are independently non-negatively charged internucleotidic linkages. In some embodiments, both the first and the last internucleotidic linkages are independently phosphoryl guanidine internucleotidic linkages. In some embodiments, both the first and the last internucleotidic linkages are independently n001. In some embodiments, they are both chirally controlled and are Rp. In some embodiments, an oligonucleotide comprises a nucleoside N₀ which comprises a natural DNA sugar (two 2′-H), a natural RNA sugar or a 2′-F modified sugar. In some embodiments, N₀ is a nucleoside opposite to a target adenosine when an oligonucleotide is utilized for adenosine editing. In some embodiments, sugar of N₀ is a natural DNA sugar. In some embodiments, sugar of N₁(“+” or nothing before a number indicates counting toward the 5′-direction (5′ . . . N₁N₀N₋₁ . . . 3′)) is a 2′-F modified sugar, a natural DNA sugar, or a natural RNA sugar. In some embodiments, sugar of N₁ is a DNA sugar. In some embodiments, sugar of N₋₁ (“−” indicates counting toward the 3′-direction (5′ . . . N₁N₀N₋₁ . . . 3′)) is a 2′-F modified sugar, a natural DNA sugar, or a natural RNA sugar. In some embodiments, sugar of N₋₁ is a DNA sugar. In some embodiments, sugar of N₋₃ is a 2′-F modified sugar. In some embodiments, between N₂ and their 5′-ends oligonucleotides comprise multiple 2′-F modified sugars and multiple 2′-modified sugars (e.g., 2′-OR modified sugars wherein R is optionally substituted C₁₋₆ alkyl, bicyclic sugars such as LNA sugars, cEt sugars, etc.). In some embodiments, oligonucleotides comprise one or more (e.g., 1-20, 1-15, 1-10, 2-15, 2-10, or 1, 2, 3, 4, 5, 6, 7, 8, 9, 10, 11, 12, 13, 14, 15, 16, 17, 18, 19, 20 or more) 2′-F blocks and one or more (e.g., 1-20, 1-15, 1-10, 2-15, 2-10, or 1, 2, 3, 4, 5, 6, 7, 8, 9, 10, 11, 12, 13, 14, 15, 16, 17, 18, 19, 20 or more) separating blocks from N₂ to their 5′-ends (e.g., first domains and first subdomains of second domains combined when first subdomains end with and include N₂), wherein each nucleoside in a 2′-F block independently comprises a 2′-F modification, each nucleoside in a separating block independently comprises no 2′-F modification, and each block independently comprises one or more (e.g., 1-20, 1-15, 1-10, 2-15, 2-10, or 1, 2, 3, 4, 5, 6, 7, 8, 9, 10, 11, 12, 13, 14, 15, 16, 17, 18, 19, 20 or more) nucleosides. In some embodiments, there are two or more such 2′-F blocks and two or more such separating blocks. In some embodiments, one or more or all such separating blocks are independently bonded to two 2′-F blocks. In some embodiments, each nucleoside in one or more or all separating blocks independently comprise a 2′-OR modification wherein R is optionally substituted C₁₋₆ alkyl or is a bicyclic sugar such as a LNA sugar, a cEt sugar, etc. In some embodiments, each nucleoside in one or more or all separating blocks independently comprise a 2′-OR modification wherein R is optionally substituted C₁₋₆ alkyl. In some embodiments, each nucleoside in one or more or all separating blocks independently comprise a 2′-OMe or 2′-MOE modification. In some embodiments, each of such 2′-F and separating blocks independently comprises 1, 2, 3, 4 or 5 nucleosides. In some embodiments, nucleosides close to N₀, e.g., N₂, N₁, N₀, N₋₁, N₋₂, etc., do not contain large 2′-modifications such as 2′-MOE. In some embodiments, sugars of N₂, N₁, N₀, N₋₁, and N₋₂ are independently natural DNA sugar, 2′-F modified sugar, or 2′-OMe modified sugar. In some embodiments, sugars of N₁, N₀, N₋₁ are each a natural DNA sugar. In some embodiments, each chiral internucleotidic linkage is independently chirally controlled.

In some embodiments, a first domain comprises one or more 2′-F modifications, and a second domain comprises one or more sugars that do not have a 2′-F modification. In some embodiments, a provided oligonucleotide comprises one or more chiral modified internucleotidic linkages. In some embodiments, a first domain comprises at least 1, 2, 3, 4, 5, 6, 7, 8, 9, 10, 11, 12, 13, 14, 15, 16, 17, 18, 19, or 20 or more sugars comprising a 2′-F modification and 1, 2, 3, 4, 5, 6, 7, 8, 9, 10, 11, 12, 13, 14, 15, 16, 17, 18, 19, or 20 or more sugars each independently comprising a 2′-OR modification wherein R is not —H (e.g., 2′-OMe, 2,-MOE, 2′-O-L^{B}-4′ wherein L^{B} is optionally substituted —CH₂—, etc.). In some embodiments, a second domain comprises at least 1, 2, 3, 4, 5, 6, 7, 8, 9, 10, 11, 12, 13, 14, 15, 16, 17, 18, 19, or 20 or more sugars each independently comprising a 2′-OR modification wherein R is not —H (e.g., 2′-OMe, 2,-MOE, 2′-O-L^{B}-4, wherein L^{B}is optionally substituted —CH₂—, etc.).

In some embodiments, about 20%-80% (e.g., about 25%-80%, 30%-80%, 35%-80%, 40%-80%, 40%-70%, 40%-60%, 50%-80%, 50%-75%, 50%-60%, 55%-80%, 60-80%, or about 50%,55%, 60%, 65%, 70%, 75%, or 80%) of all sugars of a first domain comprises a 2′-F modification. In some embodiments, about 20%-70% (e.g., about 20%-60%, 20%-50%, 30%-60%, 30%-50%, 40%-50%, or about 20%, 25%, 30%, 35%, 40%, 45%, 50%, 55%, or 60%) of all sugars of a first domain independently comprises a 2′-OR modifications wherein R is not —H (e.g., 2′-OMe, 2,-MOE, 2′-O-L^{B}-4′ wherein L^{B} is optionally substituted —CH₂—, etc.). In some embodiments, a second domain comprises at least 1, 2, 3, 4, 5, 6, 7, 8, 9, 10, 11, 12, 13, 14, 15, 16, 17, 18, 19, or 20 or more modified sugars comprising no 2′-F modification, or at least 50%, 60%, 70%, 75%, 80%, 85%, 90%, 95%, or 99% of all sugars of the second domain comprise no 2′-F modification.

In some embodiments, a second domain comprises or consists of a first subdomain, a second subdomain and a third subdomain as described herein. In some embodiments, a first subdomain comprises one or more (e.g., 1-10, 1-5, 1-3, 1, 2, 3, 4, 5, 6, 7, 8, 9, or 10) sugars each independently comprising a 2′-OR modification wherein R is not —H (e.g., 2′-OMe, 2,-MOE, 2′-O-L^{B}-4′ wherein L^{B}is optionally substituted —CH₂—, etc.). In some embodiments, there are more such sugars in a first subdomain than 2′-F modified sugars. In some embodiments, none of sugars in a second subdomain contain any 2′-OR modifications wherein R is optionally substituted C₁₋₆ aliphatic or 2′-O-L^{B}-4′). In some embodiments, each sugar of a second subdomain is independently a natural DNA sugar, a natural RNA sugar or a 2′-F modified sugar. In some embodiments, each sugar of a second subdomain is independently a natural DNA sugar or a natural RNA sugar. In some embodiments, each sugar of a second subdomain is independently a natural DNA sugar or a 2′-F modified sugar. In some embodiments, each sugar of a second subdomain is independently a natural DNA sugar. In some embodiments, there are three nucleosides in a second subdomain. In some embodiments, when binding to a target the second nucleoside the three is opposite to a target adenosine. In some embodiments, the sugar of a second nucleoside does not contain any 2′-OR modifications as described herein (e.g., 2′-OMe, 2′-MOE etc.). In some embodiments, such a sugar is a natural DNA sugar. In some embodiments, it is a natural RNA sugar. In some embodiments, it is a 2′-F modified sugar. In some embodiments, a third subdomain comprises one or more (e.g., 1-10, 1-5, 1-3, 1, 2, 3, 4, 5, 6, 7, 8, 9, or 10) sugars each independently comprising a 2′-OR modification wherein R is not —H (e.g., 2′-OMe, 2,-MOE, 2′-O-L^{B}-4, wherein L^{B}is optionally substituted —CH₂—, etc.). In some embodiments, there are more such sugars in a third subdomain than 2′-F modified sugars.

In some embodiments, a second domain comprises at least 1, 2, 3, 4, 5, 6, 7, 8, 9, 10, 11, 12, 13, 14, 15, 16, 17, 18, 19, or 20 or more modified sugars independently comprising a 2′-OR modification, or at least 50%, 60%, 70%, 75%, 80%, 85%, 90%, 95%, or 99% of all sugars of a second domain comprise a 2′-OR modification, wherein R is optionally substituted C₁₋₆ aliphatic. In some embodiments, R is methyl. In some embodiments, R is —CH₂CH₂OCH₃. As described herein, other sugar modifications may also be utilized in accordance with the present disclosure, optionally with base modifications and/or internucleotidic linkage modifications described herein.

In some embodiments, an oligonucleotide comprises or is of a 5′-first domain-second domain-3′ structure. In some embodiments, a second domain comprises or is of a 5′-first subdomain-second subdomain-third subdomain-3′ structure. In some embodiments, an oligonucleotide comprises or is of a 5′-first domain-first subdomain-second subdomain-third subdomain-3′ structure. In some embodiments, oligonucleotide is conjugated to an additional moiety, e.g., various additional chemical moieties as described herein. In some embodiments, an oligonucleotide comprises an additional moiety, e.g., an additional moiety as described herein. In some embodiments, an additional chemical moiety is or comprises a small molecule moiety, a carbohydrate moiety (e.g., GalNAc moiety), a nucleic acid moiety (e.g., an oligonucleotide moiety, a nucleic acid moiety which can provide and/or modulate one or more properties and/or activities, etc. (e.g., a moiety of RNase H-dependent oligonucleotide, RNAi oligonucleotide, aptamer, gRNA, etc.), and/or a peptide moiety.

In some embodiments, base sequence of a provided oligonucleotide is substantially complementary to the base sequence of a target nucleic acid comprising a target adenosine. In some embodiments, a provided oligonucleotide when aligned to a target nucleic acid comprises one or more mismatches (non-Watson-Crick base pairs). In some embodiments, a provided oligonucleotide when aligned to a target nucleic acid comprises one or more wobbles (e.g., G-U, I-A, G-A, I-U, I-C, etc.). In some embodiments, mismatches and/or wobbles may help one or more proteins, e.g., ADAR1, ADAR2, etc., to recognize a duplex formed by a provided oligonucleotide and a target nucleic acid. In some embodiments, provided oligonucleotides form duplexes with target nucleic acids. In some embodiments, ADAR proteins recognize and bind to such duplexes. In some embodiments, nucleosides opposite to target adenosines are located in the middle of provided oligonucleotides, e.g., with 5-50 nucleosides to 5′ side, and 1-50 nucleosides on its 3′ side. In some embodiments, a 5′ side has more nucleosides than a 3′ side. In some embodiments, a 5′ side has fewer nucleosides than a 3′ side. In some embodiments, a 5′ side has the same number of nucleosides as a 3′ side.

In some embodiments, identity, e.g., of two nucleic acid sequences, is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about 100%.

In some embodiments, with utilization of various structural elements (e.g., various modifications, stereochemistry, and patterns thereof), the present disclosure can achieve desired properties and high activities with short oligonucleotides, e.g., those of about 20-40, 25-40, 25-35, 26-32, 25, 26, 27, 28, 29, 30, 31, 32 33, 34 or 35 nucleobases in length.

In some embodiments, provided oligonucleotides comprise modified nucleobases. In some embodiments, a modified nucleobase promotes modification of a target adenosine. In some embodiments, a nucleobase which is opposite to a target adenine maintains interactions with an enzyme, e.g., ADAR, compared to when a U is present, while interacts with a target adenine less strongly than U (e.g., forming fewer hydrogen bonds). In some embodiments, an opposite nucleobase and/or its associated sugar provide certain flexibility (e.g., when compared to U) to facility modification of a target adenosine by enzymes, e.g., ADAR1, ADAR2, etc. In some embodiments, a nucleobase immediately 5′ or 3′ to the opposite nucleobase (to a target adenine), e.g., I and derivatives thereof, enhances modification of a target adenine. Among other things, the present disclosure recognizes that such a nucleobase may causes less steric hindrance than G when a duplex of a provided oligonucleotide and its target nucleic acid interact with a modifying enzyme, e.g., ADAR1 or ADAR2. In some embodiments, base sequences of oligonucleotides are selected (e.g., when several adenosine residues are suitable targets) and/or designed (e.g., through utilization of various nucleobases described herein) so that steric hindrance may be reduced or removed (e.g., no G next to the opposite nucleoside of a target A).

Various internucleotidic linkages may be utilized in oligonucleotides in accordance with the present disclosure. In some embodiments, an oligonucleotide comprises one or more types of internucleotidic linkage. In some embodiments, an oligonucleotide comprises two or more types of internucleotidic linkage. In some embodiments, an oligonucleotide comprises at least three types of internucleotidic linkages. In some embodiments, a linkage contains a linkage phosphorus atom bonded to an oxygen atom which oxygen atom is not bonded to or is not part of a backbone sugar (“a PO linkage”, e.g., a natural phosphate linkage). In some embodiments, a linkage contains a linkage phosphorus atom bonded to a sulfur atom which sulfur atom is not bonded to or is not part of a backbone sugar (“a PS linkage”, e.g., a phosphorothioate internucleotidic linkage). In some embodiments, a linkage contains a linkage phosphorus atom bonded to a nitrogen atom which nitrogen atom is not bonded to or is not part of a backbone sugar (“a PN linkage”, e.g., n001). In some embodiments, an oligonucleotide comprises one or more PS linkages. In some embodiments, an oligonucleotide comprises one or more PO linkages. In some embodiments, an oligonucleotide comprises one or more PN linkages. In some embodiments, an oligonucleotide comprises one or more PS and one or more PO linkages. In some embodiments, an oligonucleotide comprises one or more PS and one or more PN linkages. In some embodiments, an oligonucleotide comprises one or more PS, one or more PN and one or more PO linkages.

In some embodiments, a first domain comprises one or more PO linkages, one or more PS linkages and one or more PN linkages. In some embodiments, a first subdomain comprises one or more PO linkages, one or more PS linkages and/or one or more PN linkages. In some embodiments, a first subdomain comprises one or more PO linkages. In some embodiments, a first subdomain comprises one or more natural phosphate linkages. In some embodiments, second subdomain comprises one or more modified internucleotidic linkages. In some embodiments, each internucleotidic linkage bonded to a nucleoside of a second subdomain is independently a modified internucleotidic linkage. In some embodiments, each internucleotidic linkage bonded to a nucleoside of a second subdomain is independently a PS or PN linkages. In some embodiments, a third subdomain comprises one or more PO linkages, one or more PS linkages and/or one or more PN linkages. In some embodiments, a third subdomain comprises one or more PO linkages. In some embodiments, a third subdomain comprises one or more natural phosphate linkages. In some embodiments, a third subdomain comprises one or more PS linkages. In some embodiments, a third subdomain comprises one or more PN linkages. In some embodiments, a third subdomain comprises one or more PO linkages, one or more PS linkages and one or more PN linkages. In some embodiments, the first internucleotidic linkage of a first domain or an oligonucleotide is a PN linkage. In some embodiments, the last internucleotidic linkage of a third subdomain or an oligonucleotide is a PN linkage. In some embodiments, a natural DNA sugar is bonded to a modified internucleotidic linkage. In some embodiments, a natural DNA sugar is bonded to a PN or PS internucleotidic linkage. In some embodiments, each natural DNA sugar in an oligonucleotide or a portion thereof (e.g., a first domain, a first subdomain, a second subdomain, a third subdomain, etc.) is independently bonded to a modified internucleotidic linkage. In some embodiments, each natural DNA sugar is independently bonded to a PN or PS internucleotidic linkage. In some embodiments, a natural RNA sugar is bonded to a modified internucleotidic linkage. In some embodiments, a natural RNA sugar is bonded to a PN or PS internucleotidic linkage. In some embodiments, each natural RNA sugar in an oligonucleotide or a portion thereof (e.g., a first domain, a first subdomain, a second subdomain, a third subdomain, etc.) is independently bonded to a modified internucleotidic linkage. In some embodiments, each natural RNA sugar is independently bonded to a PN or PS internucleotidic linkage.

In some embodiments, a 2′-F modified sugar is bonded to a modified internucleotidic linkage. In some embodiments, a 2′-F modified sugar is bonded to a PN or PS internucleotidic linkage. In some embodiments, each 2′-F modified sugar in an oligonucleotide or a portion thereof (e.g., a first domain, a first subdomain, a second subdomain, a third subdomain, etc.) is independently bonded to a modified internucleotidic linkage. In some embodiments, each 2′-F modified sugar is independently bonded to a PN or PS internucleotidic linkage. In some embodiments, each PO linkage is independently a natural phosphate linkage. In some embodiments, each PS linkage is independently a phosphorothioate internucleotidic linkage. In some embodiments, one or more PN linkages are independently non-negatively charged internucleotidic linkage. In some embodiments, one or more PN linkages are independently neutral internucleotidic linkage. In some embodiments, one or more PN linkages are independently phosphoryl guanidine linkages. In some embodiments, each PN linkage is independently a phosphoryl guanidine linkage. In some embodiments, one or more PN linkages are independently n001. In some embodiments, each PN linkage is independently n001.

In some embodiments, oligonucleotides of the present disclosure provides modified internucleotidic linkages (i.e., internucleotidic linkages that are not natural phosphate linkages). In some embodiments, linkage phosphorus of modified internucleotidic linkages (e.g., chiral internucleotidic linkages) are chiral and can exist in different configurations (Rp and Sp). In some embodiments, incorporation of modified internucleotidic linkage, particularly with control of stereochemistry of linkage phosphorus centers (so that at such a controlled center one configuration is enriched compared to stereorandom oligonucleotide preparation), can significantly improve properties (e.g., stability) and/or activities (e.g., adenosine modifying activities (e.g., converting an adenosine to inosine). In some embodiments, provided oligonucleotides have stereochemical purity significantly higher than stereorandom preparations. In some embodiments, provided oligonucleotides are chirally controlled.

In some embodiments, oligonucleotides of the present disclosure comprise one or more chiral internucleotidic linkages whose linkage phosphorus is chiral (e.g., a phosphorothioate internucleotidic linkage). In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% (e.g., 50%-100%, 60%-100%, 70-100%, 75%-100%, 80%-100%, 90%-100%, 95%-100%, 60%-95%, 70%-95%, 75-95%, 80-95%, 85-95%, 90-95%, 50%, 60%, 70%, 75%, 80%, 85%, 90%, 95%, or 99%, etc.) of all, or all internucleotidic linkages in an oligonucleotide, are chiral internucleotidic linkages. In some embodiments, at least one internucleotidic linkage is a chiral internucleotidic linkage. In some embodiments, at least one internucleotidic linkage is a natural phosphate linkage. In some embodiments, each internucleotidic linkage is independently a chiral internucleotidic linkage. In some embodiments, at least one chiral internucleotidic linkage is a phosphorothioate internucleotidic linkage. In some embodiments, each is a phosphorothioate internucleotidic linkage. In some embodiments, one or more chiral internucleotidic linkages are independently a non-negatively charged internucleotidic linkage or a neutral internucleotidic linkage. In some embodiments, one or more chiral internucleotidic linkages are independently a phosphoryl guanidine internucleotidic linkage. In some embodiments, one or more chiral internucleotidic linkages are independently chirally controlled. In some embodiments, each chiral internucleotidic linkage is independently chirally controlled. In some embodiments, one or more chiral internucleotidic linkages are not chirally controlled. In some embodiments, each phosphorothioate internucleotidic linkage is independently chirally controlled. In some embodiments, each modified internucleotidic linkage is independently a phosphorothioate or a non-negatively charged internucleotidic linkage. In some embodiments, each modified internucleotidic linkage is independently a phosphorothioate or a neutral internucleotidic linkage. In some embodiments, each modified internucleotidic linkage is independently a phosphorothioate or a neutral internucleotidic linkage. In some embodiments, each modified internucleotidic linkage is independently a phosphorothioate or a phosphoryl guanidine internucleotidic linkage. In some embodiments, a phosphoryl guanidine internucleotidic linkage is n001. In some embodiments, each phosphoryl guanidine internucleotidic linkage is n001. In some embodiments, each non-negatively charged internucleotidic linkage is n001. In some embodiments, each neutral internucleotidic linkage is n001. In some embodiments, a modified internucleotidic linkage n002. In some embodiments, it is n006. In some embodiments, it is n020. In some embodiments, it is n004. In some embodiments, it is n008. In some embodiments, it is n025. In some embodiments, it is n026. Various modified internucleotidic linkages are described herein. A linkage phosphorus can be either Rp or Sp. In some embodiments, at least one linkage phosphorus is Rp. In some embodiments, at least one linkage phosphorus is Sp. In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% (e.g., 50%-100%, 60%-100%, 70-100%, 75%-100%, 80%-100%, 90%-100%, 95%-100%, 60%-95%, 70%-95%, 75-95%, 80-95%, 85-95%, 90-95%, 50%, 60%, 70%, 75%, 80%, 85%, 90%, 95%, or 99%, etc.) of all, or all chiral internucleotidic linkages in an oligonucleotide, are Sp. In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% (e.g., 50%-100%, 60%-100%, 70-100%, 75%-100%, 80%-100%, 90%-100%, 95%-100%, 60%-95%, 70%-95%, 75-95%, 80-95%, 85-95%, 90-95%, 50%, 60%, 70%, 75%, 80%, 85%, 90%, 95%, or 99%, etc.) of all, or all phosphorothioate internucleotidic linkages in an oligonucleotide, are Sp. In some embodiments, at least 50% of all phosphorothioate internucleotidic linkage are Sp. In some embodiments, at least 60% of all phosphorothioate internucleotidic linkage are Sp. In some embodiments, at least 70% of all phosphorothioate internucleotidic linkage are Sp. In some embodiments, at least 75% of all phosphorothioate internucleotidic linkage are Sp. In some embodiments, at least 80% of all phosphorothioate internucleotidic linkage are Sp. In some embodiments, at least 85% of all phosphorothioate internucleotidic linkage are Sp. In some embodiments, at least 90% of all phosphorothioate internucleotidic linkage are Sp. In some embodiments, at least 95% of all phosphorothioate internucleotidic linkage are Sp. In some embodiments, at least 96% of all phosphorothioate internucleotidic linkage are Sp. In some embodiments, at least 97% of all phosphorothioate internucleotidic linkage are Sp. In some embodiments, at least 98% of all phosphorothioate internucleotidic linkage are Sp. In some embodiments, all phosphorothioate internucleotidic linkage are Sp. In some embodiments, no more than 3, 4, 5, 6, 7, 8, 9, or 10 consecutive phosphorothioate internucleotidic linkages are Rp. In some embodiments, no more than 3 consecutive phosphorothioate internucleotidic linkages are Rp. In some embodiments, no more than 4 consecutive phosphorothioate internucleotidic linkages are Rp. In some embodiments, no more than 5 consecutive phosphorothioate internucleotidic linkages are Rp. In some embodiments, no more than 6 consecutive phosphorothioate internucleotidic linkages are Rp. In some embodiments, no more than 7 consecutive phosphorothioate internucleotidic linkages are Rp. In some embodiments, no more than 8 consecutive phosphorothioate internucleotidic linkages are Rp. In some embodiments, no more than 9 consecutive phosphorothioate internucleotidic linkages are Rp. In some embodiments, no more than 10 consecutive phosphorothioate internucleotidic linkages are Rp. In some embodiments, consecutive Rp phosphorothioate internucleotidic linkages are not utilized in portions wherein the majority (e.g., greater than 50%, 60%, 70%, 75%, 80%, 85%, 90%, 95% or more) or all of sugars are natural DNA and/or RNA and/or 2′-F modified sugars. In some embodiments, when consecutive Rp phosphorothioate internucleotidic linkages are utilized, one or more or the majority (e.g., greater than 50%, 60%, 70%, 75%, 80%, 85%, 90%, 95% or more) or all of such internucleotidic linkages are independently bonded to sugars which can improve stability. In some embodiments, when consecutive Rp phosphorothioate internucleotidic linkages are utilized, one or more or the majority (e.g., greater than 50%, 60%, 70%, 75%, 80%, 85%, 90%, 95% or more) or all of such internucleotidic linkages are independently bonded to bicyclic sugars or 2′-OR modified sugars wherein R is optionally substituted C₁₋₆ aliphatic. In some embodiments, when consecutive Rp phosphorothioate internucleotidic linkages are utilized, one or more or the majority (e.g., greater than 50%, 60%, 70%, 75%, 80%, 85%, 90%, 95% or more) or all of such internucleotidic linkages are independently bonded to 2′-OR modified sugars wherein R is optionally substituted C₁₋₆ aliphatic. In some embodiments, each 2′-OR modified sugar is independently a 2′-OMe modified sugar or a 2′-MOE modified sugar. In some embodiments, each 2′-OR modified sugar is independently a 2′-OMe modified sugar. In some embodiments, each 2′-OR modified sugar is independently a 2′-MOE modified sugar.

In some embodiments, stereochemistry of one or more chiral linkage phosphorus of provided oligonucleotides are controlled in a composition. In some embodiments, the present disclosure provides a composition comprising a plurality of oligonucleotides, wherein oligonucleotides of a plurality share a common base sequence, and the same configuration of linkage phosphorus (e.g., all are Rp or all are Sp for the chiral linkage phosphorus) independently at one or more (e.g., about 1-50, 1-40, 1-30, 1-25, 1-20, 1-15, 1-10, 5-50, 5-40, 5-30, 5-25, 5-20, 5-15, 5-10, 1,2,3,4,5, 6,7, 8, 9, 10, 11, 12, 13, 14, 15, 16, 17, 18, 19, 20, 21, 22, 23, 24, or 25 or more, or at least 50%, 60%, 70%, 75%, 80%, 85%, 90%, 95%, or 99% of all chiral internucleotidic linkages) chiral internucleotidic linkages (“chirally controlled internucleotidic linkages”). In some embodiments, they share the same stereochemistry at each chiral linkage phosphorus. In some embodiments, oligonucleotides of a plurality share the same constitution. In some embodiments, oligonucleotides of a plurality are structurally identical except the internucleotidic linkages. In some embodiments, oligonucleotides of a plurality are structurally identical. In some embodiments, at least at least about 50%, 60%, 65%, 70%, 75%, 80%, 85%, 90%, or 95% of all oligonucleotides in a composition, or of all oligonucleotides sharing the common base sequence, share the pattern of backbone chiral centers of oligonucleotides of the plurality. In some embodiments, at least about 50%, 60%, 65%, 70%, 75%, 80%, 85%, 90%, or 95% of all oligonucleotides in a composition, or of all oligonucleotides sharing the common base sequence, are oligonucleotides of the plurality.

In some embodiments, the present disclosure provides a chirally controlled oligonucleotide composition of an oligonucleotide, wherein at least about 50%, 60%, 65%, 70%, 75%, 80%, 85%, 90%, or 95% of all oligonucleotides in a composition, or of all oligonucleotides having the same base sequence of the oligonucleotide, or of all oligonucleotide having the same base sequence and sugar and base modifications, or of all oligonucleotides of the same constitution, share the same configuration of linkage phosphorus (e.g., all are Rp or all are Sp for the chiral linkage phosphorus) independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6,7, 8, 9, 10, 11, 12, 13, 14, 15, 16, 17, 18, 19, 20, 21, 22, 23, 24, or 25 or more, or at least 50%, 60%, 70%, 75%, 80%, 85%, 90%, 95%, or 99% of all chiral internucleotidic linkages) chiral internucleotidic linkages with the oligonucleotide. In some embodiments, the present disclosure provides a chirally controlled oligonucleotide composition of an oligonucleotide, wherein at least about 50%, 60%, 65%, 70%, 75%, 80%, 85%, 90%, or 95% of all oligonucleotides in a composition, or of all oligonucleotides having the same base sequence of the oligonucleotide, or of all oligonucleotide having the same base sequence and sugar and base modifications, or of all oligonucleotides of the same constitution, are one or more forms of the oligonucleotide (e.g., acid forms, salt forms (e.g. pharmaceutically acceptable salt forms; as appreciated by those skilled in the art, in case the oligonucleotide is a salt, other salt forms of the corresponding acid or base form of the oligonucleotide), etc.).

In some embodiments, chirally controlled oligonucleotide compositions provide a number of advantages, e.g., higher stability, activities, etc., compared to corresponding stereorandom oligonucleotide compositions. In some embodiments, chirally controlled oligonucleotide compositions provide high levels of adenosine modifying (e.g., converting A to I) activities with various isoforms of an ADAR protein (e.g., p150 and p110 forms of ADAR1) while corresponding stereorandom compositions provide high levels of adenosine modifying (e.g., converting A to I) activities with only certain isoforms of an ADAR protein (e.g., p150 isoform of ADAR1).

In some embodiments, provided oligonucleotides comprise an additional moiety, e.g., a targeting moiety, a carbohydrate moiety, etc. In some embodiments, an additional moiety is or comprises a ligand for an asialoglycoprotein receptor. In some embodiments, an additional moiety is or comprises GalNAc or derivatives thereof Among other things, additional moieties may facilitate delivery to certain target locations, e.g., cells, tissues, organs, etc. (e.g., locations comprising receptors that interact with additional moieties). In some embodiments, additional moieties facilitate delivery to liver.

In some embodiments, the present disclosure provides technologies for preparing oligonucleotides and compositions thereof, particularly chirally controlled oligonucleotide compositions. In some embodiments, provided oligonucleotides and compositions thereof are of high purity. In some embodiments, oligonucleotides of the present disclosure are at least 50%, 60%, 70%, 75%, 80%, 85%, 90%, 95%, or 99% stereochemically pure at linkage phosphorus of chiral internucleotidic linkages. In some embodiments, oligonucleotides of the present disclosure are prepared stereoselectively and are substantially free of stereoisomers. In some embodiments, in provided compositions comprising a plurality of oligonucleotides which share the same base sequence of the same pattern of chiral linkage phosphorus stereochemistry (e.g., comprising one or more of Rp and/or Sp, wherein each chiral linkage phosphorus is independently Rp or Sp), at least 50%, 60%, 70%, 75%, 80%, 85%, 90%, 95%, or 99% of all oligonucleotides in the composition that share the same base sequence as oligonucleotides of the plurality share the same pattern of chiral linkage phosphorus stereochemistry or are oligonucleotides of the plurality. In some embodiments, in provided compositions comprising a plurality of oligonucleotides which share the same base sequence of the same pattern of chiral linkage phosphorus stereochemistry, at least 50%, 60%, 70%, 75%, 80%, 85%, 90%, 95%, or 99% of all oligonucleotides in the composition that share the same constitution as oligonucleotides of the plurality share the same pattern of chiral linkage phosphorus stereochemistry or are oligonucleotides of the plurality.

In some embodiments, the present disclosure describes useful technologies for assessing oligonucleotide and compositions thereof. For example, various technologies of the present disclosure are useful for assessing adenosine modification. As appreciated by those skilled in the art, in some embodiments, modification/editing of adenosine can be assessed through sequencing, mass spectrometry, assessment (e.g., levels, activities, etc.) of products (e.g., RNA, protein, etc.) of modified nucleic acids (e.g., wherein adenosines of target nucleic acids are converted to inosines), etc., optionally in view of other components (e.g., ADAR proteins) presence in modification systems (e.g., an in vitro system, an ex vivo system, cells, tissues, organs, organisms, subjects, etc.). Those skilled in the art will appreciate that oligonucleotides which provide adenosine modification of a target nucleic acid can also provide modified nucleic acid (e.g., wherein a target adenosine is converted into I) and one or more products thereof (e.g., mRNA, proteins, etc.). Certain useful technologies are described in the Examples.

As described herein, oligonucleotides and compositions of the present disclosure may be provided/utilized in various forms. In some embodiments, the present disclosure provides compositions comprising one or more forms of oligonucleotides, e.g., acid forms (e.g., in which natural phosphate linkages exist as —O(P(O)(OH)—O—, phosphorothioate internucleotidic linkages exist as —O(P(O)(SH)—O—), base forms, salt forms (e.g., in which natural phosphate linkages exist as salt forms (e.g., sodium salt (—O(P(O)(O—Na⁻)—O—), phosphorothioate internucleotidic linkages exist as salt forms (e.g., sodium salt (—O(P(O)(S—Na⁺)—O—) etc. As appreciated by those skilled in the art, oligonucleotides can exist in various salt forms, including pharmaceutically acceptable salts, and in solutions (e.g., various aqueous buffering system), cations may dissociate from anions. In some embodiments, the present disclosure provides a pharmaceutical composition comprising a provided oligonucleotide and/or one or more pharmaceutically acceptable salts thereof, and a pharmaceutically acceptable carrier. In some embodiments, pharmaceutical compositions are chirally controlled oligonucleotide compositions.

Provided technologies can be utilized for various purposes. For example, those skilled in the art will appreciate that provided technologies are useful for many purposes involving modification of adenosine, e.g., correction of G to A mutations, modulate levels of certain nucleic acids and/or products encoded thereby (e.g., reducing levels of proteins by introducing A to G/I modifications), modulation of splicing, modulation of translation (e.g., modulating translation start and/or stop site by introducing A to G/I modifications), etc.

In some embodiments, the present disclosure provides technologies for preventing or treating a condition, disorder or disease that is amenable to an adenosine modification, e.g. conversion of A to I or G. As appreciated by those skilled in the art, I may perform one or more functions of G, e.g., in base pairing, translation, etc. In some embodiments, a G to A mutation may be corrected through conversion of A to I so that one or more products, e.g., proteins, of the G-version nucleic acid can be produced. In some embodiments, the present disclosure provides technologies for preventing or treating a condition, disorder or disease associated with a mutation, comprising administering to a subject susceptible thereto or suffering therefrom a provided oligonucleotide or composition thereof, which oligonucleotide or composition can edit a mutation. In some embodiments, the present disclosure provides technologies for preventing or treating a condition, disorder or disease associated with a G to A mutation, comprising administering to a subject susceptible thereto or suffering therefrom a provided oligonucleotide or composition thereof, which oligonucleotide or composition can modify an A. In some embodiments, provided technologies modify an A in a transcript, e.g., RNA transcript. In some embodiments, an A is converted into an I. In some embodiments, during translation protein synthesis machineries read I as G. In some embodiments, G/I forms have and/or encode one or more proteins that have one or more higher desired activities and/or one or more better desired properties compared to the corresponding A forms and/or one or more proteins encoded thereby. In some embodiments, G/I forms provide higher levels, compared to the corresponding A forms, of one or more proteins that have one or more desired activities, one or more desired levels of activities, and/or one or more better desired properties. In some embodiments, products encoded by an G/I form are structurally different (e.g., longer, in some embodiments, full length proteins) from those encoded by its corresponding A form. In some embodiments, G/I forms provide structurally identical products (e.g., proteins) compared to the corresponding A forms but G/I forms provide such products at more desired levels.

As those skilled in the art will appreciate, many conditions, disorders or diseases are associated with mutations that can be modified by provided technologies and can be prevented and/or treated using provided technologies. For example, it is reported that there are over 20,000 conditions, disorders or diseases are associated with G to A mutation and can benefit from A to I editing.

### BRIEF DESCRIPTION OF THE DRAWINGS

FIG. 1. Provided technologies can provide editing. Various oligonucleotides comprising first and second domains whose corresponding first and second portions are separated by gaps of various lengths (in some embodiments, 1, 3, 5, 10, 30, 60, 137, 200, or 206 nucleobases) were assessed. As shown in (A) and (B), various oligonucleotides can provide editing of the target adenosine. Oligonucleotides were transfected into 293T cells that either expressed ADAR-110 (A) or ADAR-150 (B).

FIG. 2. Provided technologies can provide editing. Various oligonucleotides comprising first and second domains whose corresponding first and second portions in a NRF2 transcript are separated by gaps of various lengths were assessed. Various oligonucleotides can provide editing of a target adenosine in NRF2 transcripts. Oligonucleotides were transfected into human primary astrocytes expressing endogenous ADAR. Gap lengths are indicated in parentheses; canonical=no gap. Error bars represent standard error of the mean (SEM), N=2.

FIG. 3. Provided technologies can provide editing. Various oligonucleotides comprising a first domain complementary to a first target adenosine and a second domain complementary to a second target adenosine can provide editing of target adenosines in NRF2 transcripts. Oligonucleotides were transfected into human primary astrocytes expressing endogenous ADAR. Target adenosines are referred to by the associated amino acid codon (e.g., Q26 indicates editing of an adenosine within the glutamine codon corresponding to position 26 of the NRF2 protein). Position of nucleoside opposite of the target adenosine is indicated in parentheses (e.g., P10 indicates nucleoside 10 of oligonucleotide, when counting from 5′-to-3′). Error bars represent standard error of the mean (SEM), N=2.

FIG. 4. Provided technologies can provide editing. Various oligonucleotides comprising first and second domains whose corresponding first and second portions are separated by gaps of various lengths were assessed. Various oligonucleotides can provide editing of a target adenosine in Malat1 transcripts. HEK293T cells were transfected with a plasmid encoding ADAR1 p110 for exogenous ADAR expression. Oligonucleotides were transfected into cells 24 hours later at a dosage of 25 nM. Gap lengths are indicated in parentheses; canonical=no gap. Error bars represent standard error of the mean (SEM), N=2.

FIG. 5. Provided technologies can provide editing. Various oligonucleotides comprising first and second domains whose corresponding first and second portions are separated by gaps of various lengths were assessed. As shown in (A), (B), and (C), various oligonucleotides can provide editing of a target adenosine in MECP2 transcripts. HEK293T cells were transfected with a plasmid encoding ADAR1 p110 for exogenous ADAR expression and a plasmid encoding MECP2 for exogenous expression of MECP2. Oligonucleotides were transfected into cells 24 hours later at a dosage of 25 nM. Gap lengths are indicated in parentheses. Error bars represent standard error of the mean (SEM), N=2.

FIG. 6. Provided technologies can provide editing. Various oligonucleotides comprising first and second domains whose corresponding first and second portions are separated by gaps of various lengths were assessed. Oligonucleotides comprising various linkers (e.g., [SpC3], [oC4o], [oC6o], [Sp9], [Sp12]) can provide editing of a target adenosine in MECP2 transcripts. HEK293T cells were transfected with a plasmid encoding ADAR1 p110 for exogenous ADAR expression and a plasmid encoding MECP2 for exogenous expression of MECP2. Oligonucleotides were transfected into cells 24 hours later at a dosage of 25 nM. Gap lengths of oligonucleotides are indicated at bottom. Error bars represent standard error of the mean (SEM), N=2.

FIG. 7. Provided technologies can provide editing. Various oligonucleotides comprising first and second domains whose corresponding first and second portions are separated by gaps of various lengths were assessed. Oligonucleotides comprising various linkers (e.g., [SpC3], [oC4o], [oC6o], [Sp9], [Sp12]) can provide editing of a target adenosine in Malat1 transcripts. HEK293T cells were transfected with a plasmid encoding ADAR1 p110 for exogenous ADAR expression. Oligonucleotides were transfected into cells 24 hours later at a dosage of 25 nM. Gap lengths of oligonucleotides are indicated at bottom. Error bars represent standard error of the mean (SEM), N=2.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation, which chiral auxiliaries often are part of chiral phosphoramidites used during oligonucleotide preparation. In contrast to chiral control, a person having ordinary skill in the art will appreciate that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In some embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%,20%, 30%,40%,50%,60%,70%,80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100^{%}, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%,91%,92%,93%.94%,95%,96%,97%,98%,99%, or 100%, or at least 5%, 10%,20%,30%,40%,50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%,99%, or 100%, or at least 5%, 10%,20%,30%,40%,50%,60%,70%,80%,85%,90%,91%,92%,93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality share the same pattern of sugar and/or nucleobase modifications, in any. In some embodiments, oligonucleotides (or nucleic acids) of a plurality are various forms of the same oligonucleotide (e.g., acid and/or various salts of the same oligonucleotide). In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)” °, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)” °, wherein DS is 95%-100%. For example, when DS is 99% and nc is 10, the percentage is or is at least 90% ((99%)¹⁰ 0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramiditc method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbomyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or <NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%0, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from =S, =Se, =NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral (e.g., as in phosphorothioate internucleotidic linkages). In some embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-F. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly-or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, U1 adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, an oligonucleotide is from about 9 to about 39 nucleosides in length. In some embodiments, an oligonucleotide is from about 25 to about 70 nucleosides in length. In some embodiments, an oligonucleotide is from about 26 to about 70 nucleosides in length. In some embodiments, an oligonucleotide is from about 27 to about 70 nucleosides in length. In some embodiments, an oligonucleotide is from about 28 to about 70 nucleosides in length. In some embodiments, an oligonucleotide is from about 29 to about 70 nucleosides in length. In some embodiments, an oligonucleotide is from about 30 to about 70 nucleosides in length. In some embodiments, an oligonucleotide is from about 31 to about 70 nucleosides in length. In some embodiments, an oligonucleotide is from about 32 to about 70 nucleosides in length. In some embodiments, an oligonucleotide is from about 25 to about 60 nucleosides in length. In some embodiments, an oligonucleotide is from about 25 to about 50 nucleosides in length. In some embodiments, an oligonucleotide is from about 25 to about 40 nucleosides in length. In some embodiments, an oligonucleotide is from about 30 to about 40 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, an oligonucleotide is at least 4 nucleosides in length. In some embodiments, an oligonucleotide is at least 5 nucleosides in length. In some embodiments, an oligonucleotide is at least 6 nucleosides in length. In some embodiments, an oligonucleotide is at least 7 nucleosides in length. In some embodiments, an oligonucleotide is at least 8 nucleosides in length. In some embodiments, an oligonucleotide is at least 9 nucleosides in length. In some embodiments, an oligonucleotide is at least 10 nucleosides in length. In some embodiments, an oligonucleotide is at least 11 nucleosides in length. In some embodiments, an oligonucleotide is at least 12 nucleosides in length. In some embodiments, an oligonucleotide is at least 15 nucleosides in length. In some embodiments, an oligonucleotide is at least 15 nucleosides in length. In some embodiments, an oligonucleotide is at least 16 nucleosides in length. In some embodiments, an oligonucleotide is at least 17 nucleosides in length. In some embodiments, an oligonucleotide is at least 18 nucleosides in length. In some embodiments, an oligonucleotide is at least 19 nucleosides in length. In some embodiments, an oligonucleotide is at least 20 nucleosides in length. In some embodiments, an oligonucleotide is at least 25 nucleosides in length. In some embodiments, an oligonucleotide is at least 26 nucleosides in length. In some embodiments, an oligonucleotide is at least 27 nucleosides in length. In some embodiments, an oligonucleotide is at least 28 nucleosides in length. In some embodiments, an oligonucleotide is at least 29 nucleosides in length. In some embodiments, an oligonucleotide is at least 30 nucleosides in length. In some embodiments, an oligonucleotide is at least 31 nucleosides in length. In some embodiments, an oligonucleotide is at least 32 nucleosides in length. In some embodiments, an oligonucleotide is at least 33 nucleosides in length. In some embodiments, an oligonucleotide is at least 34 nucleosides in length. In some embodiments, an oligonucleotide is at least 35 nucleosides in length. In some embodiments, an oligonucleotide is at least 36 nucleosides in length. In some embodiments, an oligonucleotide is at least 37 nucleosides in length. In some embodiments, an oligonucleotide is at least 38 nucleosides in length. In some embodiments, an oligonucleotide is at least 39 nucleosides in length. In some embodiments, an oligonucleotide is at least 40 nucleosides in length. In some embodiments, an oligonucleotide is 25 nucleosides in length. In some embodiments, an oligonucleotide is 26 nucleosides in length. In some embodiments, an oligonucleotide is 27 nucleosides in length. In some embodiments, an oligonucleotide is 28 nucleosides in length. In some embodiments, an oligonucleotide is 29 nucleosides in length. In some embodiments, an oligonucleotide is 30 nucleosides in length. In some embodiments, an oligonucleotide is 31 nucleosides in length. In some embodiments, an oligonucleotide is 32 nucleosides in length. In some embodiments, an oligonucleotide is 33 nucleosides in length. In some embodiments, an oligonucleotide is 34 nucleosides in length. In some embodiments, an oligonucleotide is 35 nucleosides in length. In some embodiments, an oligonucleotide is 36 nucleosides in length. In some embodiments, an oligonucleotide is 37 nucleosides in length. In some embodiments, an oligonucleotide is 38 nucleosides in length. In some embodiments, an oligonucleotide is 39 nucleosides in length. In some embodiments, an oligonucleotide is 40 nucleosides in length. In some embodiments, each nucleoside counted in an oligonucleotide length independently comprises a nucleobase comprising a ring having at least one nitrogen ring atom. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications. In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{o}; —(CH₂)₀₋₄R^{o}; —O(CH₂)⁰⁻⁴R^{o}, —O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄₀C(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —Si(R^{o})₃; —OSi(R^{o})₃; —B(R^{o})₂; —OB(R^{o})₂; —OB(OR^{o})₂; —P(R^{o})₂; —P(OR^{o})₂; —P(R^{o})(OR^{o}); —OP(R^{o})₂; —OP(OR^{o})₂; —OP(R^{o})(OR^{o}); —P(O)(R^{o})₂; —P(O)(OR^{o})₂; —OP(O)(R^{o})₂; —OP(O)(OR^{o})₂; —OP(O)(OR^{o})(SR^{o}); —SP(O)(R^{o})₂; —SP(O)(OR^{o})₂; —N(R^{o})P(O)(R^{o})₂; —N(R^{o})P(O)(OR^{o})₂; —P(R^{o})₂[B(R^{o})₃]; —P(OR^{o})₂[B(R^{o})₃]; —OP(R^{o})₂[B(R^{o})₃]; —OP(OR^{o})₂[B(R^{o})₃]; —(C₁₋₄ straight or branched alkylene)O—N(R^{o})₂; or —(C₁₋₄ straight or branched alkylene)C(O)O—N(R^{o})₂, wherein each R^{o} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C_{6_14} aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{o} (or the ring formed by taking two independent occurrences of R^{o} together with their intervening atoms), are independently halogen, —(CH₂)₀₋₂R^{•}, —(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below. unsubstituted —OPh. or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt. or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O—and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate. 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilvlazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N_{-1,1}-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-²-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2- trimethylsilylethyl, p-chlorophenyl, 2,4-dinitrophenyl, benzyl, benzoyl, p-phenylbenzoyl, 2,6- dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifluoroacetyl, pivaloyl, 9- fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t- butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In some embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### DESCRIPTION OF CERTAIN EMBODIMENTS

Oligonucleotides are useful in various therapeutic, diagnostic, and research applications. Use of naturally occurring nucleic acids is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities.

From a structural point of view, modifications to internucleotidic linkages can introduce chirality, and certain properties and activities may be affected by configurations of linkage phosphorus atoms of oligonucleotides. For example, binding affinity, sequence specific binding to complementary RNA, stability to nucleases, activities, delivery, pharmacokinetics, etc. can be affected by, inter alia, chirality of backbone linkage phosphorus atoms.

Among other things, the present disclosure utilizes technologies for controlling various structural elements, e.g., sugar modifications and patterns thereof, nucleobase modifications and patterns thereof, modified internucleotidic linkages and patterns thereof, linkage phosphorus stereochemistry and patterns thereof, additional chemical moieties (moieties that are not typically in an oligonucleotide chain) and patterns thereof, etc. With the capability to fully control structural elements of oligonucleotides, the present disclosure provides oligonucleotides with improved and/or new properties and/or activities for various applications, e.g., as therapeutic agents, probes, etc. For example, in some embodiments, provided oligonucleotides and compositions thereof are particularly powerful for editing target adenosine in target nucleic acids to, in some embodiments, correct a G to A mutation by converting A to I.

In some embodiments, an oligonucleotide comprises a sequence that is identical to or is completely or substantially complementary to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, typically 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, contiguous bases of a nucleic acid (e.g., DNA, pre-mRNA, mRNA, etc.). In some embodiments, a nucleic acid is a target nucleic acid comprising one or more target adenosine. In some embodiments, a target nucleic acid comprises one and no more than one target adenosine. In some embodiments, an oligonucleotide can hybridize with a target nucleic acid. In some embodiments, such hybridization facilitates modification of A (e.g., conversion of A to I) by, e.g., ADAR1, ADAR2, etc., in a nucleic acid or a product thereof.

In some embodiments, the present disclosure provides an oligonucleotide comprising:
a first domain; and
a second domain,
   wherein:
the base sequence of the first domain is complementary to a first portion of the base sequence of a target nucleic acid;
the base sequence of the second domain is complementary to a second portion of the base sequence of a target nucleic acid; and
the first portion and the second portion of the base sequence of the target nucleic acid are separated by a gap.

In some embodiments, a first domain can hybridize with a first portion of a target nucleic acid. In some embodiments, a second domain can hybridize with a second portion of a target nucleic acid. In some embodiments, a first portion and a second portion is separated by a gap. In some embodiments, the present disclosure provides an oligonucleotide comprising:
a first domain; and
a second domain,
   wherein:
the first domain is characterized in that it can form a duplex with a first portion of a target nucleic acid;
the second domain is characterized in that it can form a duplex with a second portion of a target nucleic acid; and
the first portion and the second portion of the base sequence of the target nucleic acid are separated by a gap.

In some embodiments, the length of the first domain is about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases. In some embodiments, the length of the second domain is about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases. In some embodiments, the length of the gap is about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 nucleobases. In some embodiments, the length of the gap is about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 nucleobases. In some embodiments, the length of the gap is about 2-100,000, 2-50,000, 2-10,000, 2-5,000, 3-100,000, 3-50,000, 3-10,000, 3-5,000, 4-100,000, 4-50,000, 4-10,000, 4-5,000, 5-100,000, 5-50,000, 5-10,000, 5-5,000, 10-100,000, 10-50,000, 10-10,000, 10-5,000, 20-100,000, 20-50,000, 20-10,000, 20-5,000, 50-100,000, 50-50,000, 50-10,000, 50-5,000, 100-100,000, 100-50,000, 100-10,000, or 100-5,000 nucleobases.

In some embodiments, an oligonucleotide has a base sequence which is, or comprises about 10-40, about 15-40, about 20-40, or at least about 27, at least about 28, at least about 29, at least about 30, at least about 31, at least about 32, at least about 33, at least about 34 contiguous bases of, an oligonucleotide or nucleic acid disclosed in a Table, or a sequence that is complementary to a target RNA sequence gene, transcript, etc. disclosed herein, and wherein each T can be optionally and independently replaced with U and vice versa. In some embodiments, the present disclosure provides an oligonucleotide or oligonucleotide composition as disclosed herein, e.g., in a Table. In some embodiments, base sequence of an oligonucleotide is or comprises a sequence which shares about 50%-100%, e.g., about or at least about 50%, 60%, 70%, 75%, 80%, 85%, 90%, or 95%, identity with a first domain of an oligonucleotide in a Table. Alternatively or additionally, in some embodiments, base sequence of an oligonucleotide is or comprises a sequence which shares about 50%-100%, e.g., about or at least about 50%, 60%, 70%, 75%, 80%, 85%, 90%, or 95%, identity with a second domain of an oligonucleotide in a Table. In some embodiments, base sequence of an oligonucleotide is or comprises a sequence which shares about 50%-100%, e.g., about or at least about 50%, 60%, 70%, 75%, 80%, 85%, 90%, or 95%, identity with an oligonucleotide in a Table.

In some embodiments, an oligonucleotide is a single-stranded oligonucleotide for site-directed editing of a nucleoside (e.g., a target adenosine) in a target nucleic acid, e.g., RNA.

As described herein, oligonucleotides may contain one or more modified internucleotidic linkages (non-natural phosphate linkages). In some embodiments, a modified internucleotidic linkage is a chiral internucleotidic linkage whose linkage phosphorus is chiral. In some embodiments, a modified internucleotidic linkage is a phosphorothioate internucleotidic linkage. In some embodiments, oligonucleotides comprise one or more negatively charged internucleotidic linkages (e.g., phosphorothioate internucleotidic linkages, natural phosphate linkages, etc.). In some embodiments, oligonucleotides comprise one or more non-negatively charged internucleotidic linkage. In some embodiments, oligonucleotides comprise one or more neutral internucleotidic linkage.

In some embodiments, oligonucleotides are chirally controlled. In some embodiments, oligonucleotides are chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (for a chirally pure oligonucleotide, each internucleotidic linkage is independently stereodefined or chirally controlled). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide; e.g., from traditional oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *RA A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A).

In some embodiments, oligonucleotides comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, oligonucleotides comprise one or more (e.g., 1-60, 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments. an internucleotidic linkage is a stereorandom phosphorothioate internucleotidic linkage. In some embodiments, an internucleotidic linkage is a chirally controlled phosphorothioate internucleotidic linkage.

Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75% 80% 85%, 90%, 95%, or 100%, or at least about 5%, 10%, 15%, 20%, 25%, 30%, 35%, 40%, ₄₅%, 50%, 55%, 60%, 65%, 70%, 75%, 80%, 85%, 90%, 95%, or 99% stereochemically pure.

In some embodiments, the present disclosure provides various oligonucleotide compositions. In some embodiments, oligonucleotide compositions are stereorandom or not chirally controlled. In some embodiments, there are no chirally controlled internucleotidic linkages in oligonucleotides of provided compositions. In some embodiments, internucleotidic linkages of oligonucleotides in compositions comprise one or more chirally controlled internucleotidic linkages (e.g., chirally controlled oligonucleotide compositions).

In some embodiments, an oligonucleotide composition comprises a plurality of oligonucleotides sharing a common base sequence, wherein one or more internucleotidic linkages in the oligonucleotides are chirally controlled and one or more internucleotidic linkages are stereorandom (not chirally controlled). In some embodiments, an oligonucleotide composition comprises a plurality of oligonucleotides sharing a common base sequence, wherein each internucleotidic linkage comprising chiral linkage phosphorus in the oligonucleotides is independently a chirally controlled internucleotidic linkage. In some embodiments, a plurality of oligonucleotides share the same base sequence, and the same base and sugar modification. In some embodiments, a plurality of oligonucleotides share the same base sequence, and the same base, sugar and internucleotidic linkage modification. In some embodiments, an oligonucleotide composition comprises oligonucleotides of the same constitution, wherein one or more internucleotidic linkages are chirally controlled and one or more internucleotidic linkages are stereorandom (not chirally controlled). In some embodiments, an oligonucleotide composition comprises oligonucleotides of the same constitution, wherein each internucleotidic linkage comprising chiral linkage phosphorus is independently a chirally controlled internucleotidic linkage. In some embodiments, at least 10%, 20%, 30%, 40%, 50%, 60%, 70%, 75%, 80%, 85%, 90% or 95% of all oligonucleotides, or all oligonucleotides of the common base sequence, are oligonucleotides of the plurality.

In some embodiments, the present disclosure provides technologies for preparing, assessing and/or utilizing provided oligonucleotides and compositions thereof.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some embodiments, “one or more” is one. In some embodiments, one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is one or more.

Various embodiments are described for variables, e.g., R, R^{L}, L, etc., as examples. Embodiments described for a variable, e.g., R, are generally applicable to all variables that can be such a variable (e.g., R′, R″, R^{L}, R^{L1}, etc.).

### Oligonucleotides

Among other things, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided oligonucleotides can direct A to I editing in target nucleic acids. In some embodiments, oligonucleotides of the present disclosure are single-stranded oligonucleotides capable of site-directed editing of an adenosine (conversion of A into I) in a target RNA sequence.

In some embodiments, oligonucleotides are of suitable lengths and sequence complementarity to specifically hybridize with target nucleic acids. In some embodiments, oligonucleotide is sufficiently long and is sufficiently complementary to target nucleic acids to distinguish target nucleic acid from other nucleic acids to reduce off-target effects. In some embodiments, oligonucleotide is sufficiently short to facilitate delivery, reduce manufacture complexity and/or cost which maintaining desired properties and activities (e.g., editing of adenosine).

In some embodiments, an oligonucleotide has a length of about 10-200 (e.g., about 10-20, 10-30, 10-40, 10-50, 10-60, 10-70, 10-80, 10-90, 10-100, 10-120, 10-150, 20-30, 20-40, 20-50, 20-60, 20-70, 20-80, 20-90, 20-100, 20-120, 20-150, 20-200, 25-30, 25-40, 25-50, 25-60, 25-70, 25-80, 25-90, 25-100, 25-120, 25-150, 25-200, 30-40, 30-50, 30-60, 30-70, 30-80, 30-90, 30-100, 30-120, 30-150, 30-200, 10, 20, 25, 26, 27, 28, 29, 30, 31, 32, 33, 34, 35, 36, 37, 38, 39, 40, 45, 50, 60, etc.) nucleobases. In some embodiments, the base sequence of an oligonucleotide is about 10-60 nucleobases in length. In some embodiments, a base sequence is about 15-50 nucleobases in length. In some embodiments, a base sequence is from about 15 to about 35 nucleobases in length. In some embodiments, a base sequence is from about 25 to about 34 nucleobases in length. In some embodiments, a base sequence is from about 26 to about 35 nucleobases in length. In some embodiments, a base sequence is from about 27 to about 32 nucleobases in length. In some embodiments, a base sequence is from about 29 to about 35 nucleobases in length. In some embodiments, a base sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleobases in length. In some embodiments, a base sequence is or is at least 35 nucleobases in length. In some embodiments, a base sequence is or is at least 34 nucleobases in length. In some embodiments, a base sequence is or is at least 33 nucleobases in length. In some embodiments, a base sequence is or is at least 32 nucleobases in length. In some embodiments, a base sequence is or is at least 31 nucleobases in length. In some embodiments, a base sequence is or is at least 30 nucleobases in length. In some embodiments, a base sequence is or is at least 29 nucleobases in length. In some embodiments, a base sequence is or is at least 28 nucleobases in length. In some embodiments, a base sequence is or is at least 27 nucleobases in length. In some embodiments, a base sequence is or is at least 26 nucleobases in length. In some embodiments, the base sequence of a complementary portion in a duplex is about or at least about 10, 11, 12, 13, 14, 15, 16, 17, 18, 19, 20, 21, 22, 23, 24, 25, 16, 27, 28, 29, 30, 31, 32, 33, 34, 35 or more nucleobases in length. In some embodiments, it is at least 10 nucleobases in length. In some embodiments, it is at least 11 nucleobases in length. In some embodiments, it is at least 12 nucleobases in length. In some embodiments, it is at least 13 nucleobases in length. In some embodiments, it is at least 14 nucleobases in length. In some embodiments, it is at least 16 nucleobases in length. In some embodiments, it is at least 16 nucleobases in length. In some embodiments, it is at least 17 nucleobases in length. In some embodiments, it is at least 18 nucleobases in length. In some embodiments, it is at least 19 nucleobases in length. In some embodiments, it is at least 20 nucleobases in length. In some embodiments, it is at least 21 nucleobases in length. In some embodiments, it is at least 22 nucleobases in length. In some embodiments, it is at least 23 nucleobases in length. In some embodiments, it is at least 24 nucleobases in length. In some embodiments, it is at least 25 nucleobases in length. Among other things, the present disclosure provides oligonucleotides of comparable or better properties and/or comparable or higher activities but of shorter lengths compared to prior reported adenosine editing oligonucleotides.

In some embodiments, base sequence of an oligonucleotide or a fragment thereof (e.g., a first domain, a second domain, etc.) is complementary to a base sequence of a target nucleic acid or a fragment thereof (e.g., a first portion, a second portion, etc.) with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs (AT, AU and CG). In some embodiments, there are no mismatches. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches. In some embodiments, complementarity, e.g., between oligonucleotides or a fragment thereof and target nucleic acids or a fragmentment thereof (e.g., between a first domain and a first portion, between a second domain and a second portion, etc.), is about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.). In some embodiments, complementarity is at least about 60%. In some embodiments, complementarity is at least about 65%. In some embodiments, complementarity is at least about 70%. In some embodiments, complementarity is at least about 75%. In some embodiments, complementarity is at least about 80%. In some embodiments, complementarity is at least about 85%. In some embodiments, complementarity is at least about 90%. In some embodiments, complementarity is at least about 95%. In some embodiments, complementarity is 100%. In some embodiments, complementarity is 100% except at a nucleoside opposite to a target nucleoside (e.g., adenosine) across the length of an oligonucleotide. Typically, complementarity is based on Watson-Crick base pairs AT, AU and CG. Those skilled in the art will appreciate that when assessing complementarity of two sequences of different lengths (e.g., a provided oligonucleotide and a target nucleic acid) complementarity may be properly based on the length of the shorter sequence and/or maximum complementarity between the two sequences. In many embodiments, oligonucleotides and target nucleic acids are of sufficient complementarity such that modifications are selectively directed to target adenosine sites.

In some embodiments, one or more mismatches are independently wobbles. In some embodiments, each mismatch is a wobble. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) wobbles. In some embodiments, the number is 0. In some embodiments, the number is 1. In some embodiments, the number is 2. In some embodiments, the number is 3. In some embodiments, the number is 4. In some embodiments, the number is 5. In some embodiments, a wobble is G-U, I-A, G-A, I-U, I-C, I-T, A-A, or reverse A-T. In some embodiments, a wobble is G-U, I-A, G-A, I-U, or I-C. In some embodiments, I-C may be considered a match when I is a 3′ immediate nucleoside next to a nucleoside opposite to a target nucleoside. In some embodiments, a base that forms a wobble pair (e.g., U which can form a G-U wobble) may replace a base that forms a match pair (e.g., C which matches G) and can provide oligonucleotide with editing activity.

In some embodiments, duplexes of oligonucleotides and target nucleic acids comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, distances between two mismatches, mismatches and one or both ends of oligonucleotides (or a portion thereof, e.g., first domain, second domain, first subdomain, second subdomain, third subdomain), and/or mismatches and nucleosides opposite to target adenosine can independently be 0-50, 0-40, 0-30, 0-25, 0-20, 0-15, 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleobases (not including mismatches, end nucleosides and nucleosides opposite to target adenosine). In some embodiments, a number is 0-30. In some embodiments, a number is 0-20. In some embodiments, a number is 1, 2, 3, 4, 5, 6, 7, 8, 9, 10, 11, 12, 13, 14, 15, 16, 17, 18, 19, or 20. In some embodiments, a distance between two mismatches is 0-20. In some embodiments, a distance between two mismatches is 1-10. In some embodiments, a distance between a mismatch and a 5′-end nucleoside of an oligonucleotide is 0-20. In some embodiments, a distance between a mismatch and a 5′-end nucleoside of an oligonucleotide is 5-20. In some embodiments, a distance between a mismatch and a 3′-end nucleoside of an oligonucleotide is 0-40. In some embodiments, a distance between a mismatch and a 3′-end nucleoside of an oligonucleotide is 5-20. In some embodiments, a distance between a mismatch and a nucleoside opposite to a target adenosine is 0-20. In some embodiments, a distance between a mismatch and a nucleoside opposite to a target adenosine is 1-10. In some embodiments, the number of nucleobases for a distance is 0. In some embodiments, it is 1. In some embodiments, it is 2. In some embodiments, it is 3. In some embodiments, it is 4. In some embodiments, it is 5. In some embodiments, it is 6. In some embodiments, it is 7. In some embodiments, it is 8. In some embodiments, it is 9. In some embodiments, it is 10. In some embodiments, it is 11. In some embodiments, it is 12. In some embodiments, it is 13. In some embodiments, it is 14. In some embodiments, it is 15. In some embodiments, it is 16. In some embodiments, it is 17. In some embodiments, it is 18. In some embodiments, it is 19. In some embodiments, it is 20. In some embodiments, a mismatch is at an end, e.g., a 5′-end or 3′-end of a first domain, second domain, first subdomain, second subdomain, or third subdomain. In some embodiments, a mismatch is at a nucleoside opposite to a target adenosine.

In some embodiments, provided oligonucleotides can direct adenosine editing (e.g., converting A to I) in a target nucleic acid and has a base sequence which consists of, comprises, or comprises a portion (e.g., a span of 10, 11, 12, 13, 14, 15, 16, 17, 18, 19 or more contiguous bases) of the base sequence of an oligonucleotide disclosed herein, wherein each T can be independently replaced with U and vice versa, and the oligonucleotide comprises at least one non-naturally-occurring modification of a base, sugar and/or internucleotidic linkage.

In some embodiments, a provided oligonucleotide comprises one or more carbohydrate moieties. In some embodiments, a provided oligonucleotide comprises one or more GalNAc moieties. In some embodiments, a provided oligonucleotide comprises one or more targeting moieties. Non-limiting examples of such additional chemical moieties which can be conjugated to oligonucleotide chain are described herein.

In some embodiments, provided oligonucleotides can direct a correction of a G to A mutation in a target sequence, or a product thereof. In some embodiments, a correction of a G to A mutation is or comprises conversion of A to I, which can be read as G during translation or other biological processes. In some embodiments, provided oligonucleotides can direct a correction of a G to A mutation in a target sequence or a product thereof via ADAR-mediated deamination. In some embodiments, provided oligonucleotides can direct a correction of a G to A mutation in a target sequence or a product thereof via ADAR-mediated deamination by recruiting an endogenous ADAR (e.g., in a target cell) and facilitating the ADAR-mediated deamination. Regardless, however, the present disclosure is not limited to any particular mechanism. In some embodiments, the present disclosure provides oligonucleotides, compositions, methods, etc., capable of operating via double-stranded RNA interference, single-stranded RNA interference, RNase H-mediated knock-down, steric hindrance of translation, ADAR-mediated deamination or a combination of two or more such mechanisms.

In some embodiments, an oligonucleotide comprises a structural element or a portion thereof described herein, e.g., in a Table. In some embodiments, an oligonucleotide has a base sequence which comprises the base sequence (or a portion thereof) wherein each T can be independently substituted with U, pattern of chemical modifications (or a portion thereof), and/or a format of an oligonucleotide disclosed herein, e.g., in a Table or in the Figures, or otherwise disclosed herein. In some embodiments, such oligonucleotide can direct a correction of a G to A mutation in a target sequence, or a product thereof.

Among other things, provided oligonucleotides may hybridize to their target nucleic acids (e.g., pre-mRNA, mature mRNA, etc.). In some embodiments, oligonucleotide can hybridize to a target RNA sequence nucleic acid in any stage of RNA processing, including but not limited to a pre-mRNA or a mature mRNA. In some embodiments, oligonucleotide can hybridize to any element of oligonucleotide nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR.

In some embodiments, oligonucleotide hybridizes to two or more variants of transcripts derived from a sense strand of a target site (e.g., a target sequence).

In some embodiments, provided oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, provided oligonucleotides are labeled with deuterium (replacing —¹H with —²H) at one or more positions. In some embodiments, one or more ¹H of an oligonucleotide chain or any moiety conjugated to the oligonucleotide chain (e.g., a targeting moiety, etc.) is substituted with ²H. Such oligonucleotides can be used in compositions and methods described herein.

In some embodiments, oligonucleotides comprise one or more modified nucleobases, one or more modified sugars, and/or one or more modified internucleotidic linkages as described herein. In some embodiments, oligonucleotides comprise a certain level of modified nucleobases, modified sugars, and/or modified internucleotidic linkages, e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all nucleobases, sugars, and internucleotidic linkages, respectively, within an oligonucleotide.

In some embodiments, oligonucleotides comprise one or more modified sugars. In some embodiments, an oligonucleotide comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, an oligonucleotide comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30,40 or 50, etc., about 1, 2, 3, 4, 5, 6, 7, 8, 9, 10, 11, 12, 13, 14, 15, 16, 17, 18, 19, or 20, etc.) modified sugars with 2′-F modification. In some embodiments, an oligonucleotide comprises about 2-50 (e.g., about 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., 2-40, 2-30, 2-25, 2-20, 2-15, 2-10, 3-40, 3-30, 3-25, 3-20, 3-15, 3-10, 4-40, 4-30, 4-25, 4-20, 4-15, 4-10, 5-40, 5-30, 5-25, 5-20, 5-15, 5-10, 6-40, 6-30, 6-25, 6-20, 6-15, 6-10, 7-40, 7-30, 7-25, 7-20, 7-15, 7-10, 8-40, 8-30, 8-25, 8-20, 8-15, 8-10, 9-40, 9-30, 9-25, 9-20, 9-15, 9-10, 10-40, 10-30, 10-25, 10-20, 10-15, about 2, 3, 4, 5, 6, 7, 8, 9, 10, 11, 12, 13, 14, 15, 16, 17, 18, 19, or 20, etc.) consecutive modified sugars with 2′-F modification. In some embodiments, an oligonucleotide comprises 2 consecutive 2′-F modified sugars. In some embodiments, an oligonucleotide comprises 3 consecutive 2′-F modified sugars. In some embodiments, an oligonucleotide comprises 4 consecutive 2′-F modified sugars. In some embodiments, an oligonucleotide comprises 5 consecutive 2′-F modified sugars. In some embodiments, an oligonucleotide comprises 6 consecutive 2′-F modified sugars. In some embodiments, an oligonucleotide comprises 7 consecutive 2′-F modified sugars. In some embodiments, an oligonucleotide comprises 8 consecutive 2′-F modified sugars. In some embodiments, an oligonucleotide comprises 9 consecutive 2′-F modified sugars. In some embodiments, an oligonucleotide comprises 10 consecutive 2′-F modified sugars. In some embodiments, an oligonucleotide comprises two or more 2′-F modified sugar blocks, wherein each sugar in a 2′-F modified sugar block is independently a 2′-F modified sugar. In some embodiments, each 2′-F modified sugar block independently comprises or consists of 2, 3, 4, 5, 6, 7, 8, 9, or 10 consecutive 2′-F modified sugars as described herein. In some embodiments, two consecutive 2′-F modified sugar blocks are independently separated by a separating block which separating block comprises one or more sugars that are independently not 2′-F modified sugars. In some embodiments, an oligonucleotide comprises one or more (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) 2′-F blocks and one or more (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) separating blocks. In some embodiments, a first domain comprises one or more (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) 2′-F blocks and one or more (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) separating blocks. In some embodiments, each first domain block bonded to a first domain 2′-F block is a separating block. In some embodiments, each first domain block bonded to a first domain separating block is a first domain 2′-F block. In some embodiments, each sugar in a separating block is independently not 2′-F modified. In some embodiments, two or more (e.g., 2, 3, 4, 5, 6, 7, 8, 9, or 10 or more) or all sugars in a separating block are independently not 2′-F modified. In some embodiments, a separating block comprises one or more bicyclic sugars (e.g., LNA sugar, cEt sugar, etc.) and/or one or more 2′-OR modified sugars, wherein R is optionally substituted C₁₋₆ aliphatic (e.g., 2′-OMe, 2′-MOE, etc.). In some embodiments, a separating block comprises one or more 2′-OR modified sugars, wherein R is optionally substituted C₁₋₆ aliphatic (e.g., 2′-OMe, 2′-MOE, etc.). In some embodiments, two or more non-2′-F modified sugars are consecutive. In some embodiments, two or more 2′-OR modified sugars wherein R is optionally substituted C₁₋₆ aliphatic (e.g., 2′-OMe, 2′-MOE, etc.) are consecutive. In some embodiments, a separating block comprises two or more (e.g., 2, 3, 4, 5, 6, 7, 8, 9, or 10 or more) 2′-OR modified sugars wherein R is optionally substituted C₁₋₆ aliphatic (e.g., 2′-OMe, 2′-MOE, etc.). In some embodiments, a separating block comprises two or more (e.g., 2, 3, 4, 5, 6, 7, 8, 9, or 10 or more) consecutive 2′-OR modified sugars wherein R is optionally substituted C₁₋₆ aliphatic (e.g., 2′-OMe, 2′-MOE, etc.). In some embodiments, each 2′-OR modified sugar is independently a 2′-OMe or 2′-MOE sugar. In some embodiments, each 2′-OR modified sugar is independently a 2′-OMe sugar. In some embodiments, each 2′-OR modified sugar is independently a 2′-MOE sugar. In some embodiments, a separating block comprises one or more 2′-F modified sugars. In some embodiments, none of 2′-F modified sugars in a separating block are next to each other. In some embodiments, a separating block contain no 2′-F modified sugars. In some embodiments, each sugar in a separating block is independently a 2′-OR modified sugar wherein R is optionally substituted C₁₋₆ aliphatic or a bicyclic sugar. In some embodiments, each sugar in each separating block is independently a 2′-OR modified sugar wherein R is optionally substituted C₁₋₆ aliphatic or a bicyclic sugar. In some embodiments, each sugar in a separating block is independently a 2′-OR modified sugar wherein R is optionally substituted C₁. aliphatic. In some embodiments, each sugar in each separating block is independently a 2′-OR modified sugar wherein R is optionally substituted C₁₋₆ aliphatic. In some embodiments, each sugar in a separating block is independently a 2′-OMe or 2′-MOE modified sugar. In some embodiments, each sugar in each separating block is independently a 2′-OMe or 2′-MOE modified sugar. In some embodiments, each sugar in a separating block is independently a 2′-OMe modified sugar. In some embodiments, each sugar in a separating block is independently a 2′-MOE modified sugar. In some embodiments, a separating block comprises a 2′-OMe sugar and 2′-MOE modified sugar. In some embodiments, each 2′-F block and each separating block independently contains 1, 2, 3, 4, or 5 nucleosides. In some embodiments, each 2′-F block and each separating block independently contains 1, 2, or 3 nucleosides.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are modified sugars. In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are modified sugars independently selected from 2′-F modified sugars, 2′-OR modified sugars wherein R is optionally substituted C₁₋₆ aliphatic, and bicyclic sugars (e.g., LNA sugars, cEt sugars, etc.). In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are modified sugars independently selected from 2′-F modified sugars and 2′-OR modified sugars wherein R is optionally substituted C₁₋₆ aliphatic. In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are modified sugars independently selected from 2′-F modified sugars, 2′-OMe modified sugars and 2′-MOE modified sugars. In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are modified sugars independently selected from 2′-F modified sugars and 2′-OMe modified sugars. In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-F modified sugars. In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%. In some embodiments, 10 or more (e.g., about or at least about 10, 11, 12, 13, 14, 15, 16, 17, 18, 19, 20, 21, 22, 23, 24, 25, 26, 27, 28, 29 or 30 or more, 10-50, 10-40, 10-30, 10-25, 15-50, 15-40, 15-30, 15-25, 20-50, 20-40, 20-30, 20-25, etc.) sugars are 2′-F modified sugars. In some embodiments, an oligonucleotide comprises two or more (e.g., 2-30, 2-25, 2-20, 2-15, 3-10, 3-30, 3-25, 3-20, 3-15, 3-10, 4-30, 4-25, 4-20, 4-15, 4-10, 5-30, 5-25, 5-20, 5-15, 5-10, 2, 3,4, 5, 6, 7, 8, 9, 10, 11, 12, 13, 14, 15, 16, 17, 18, 19, or 20) consecutive 2′-F modified sugars. In some embodiments, an oligonucleotide comprises one or more 2′-F blocks each independently comprising two or more (e.g., 2-30, 2-25, 2-20, 2-15, 3-10, 3-30, 3-25, 3-20, 3-15, 3-10, 4-30, 4-25, 4-20, 4-15, 4-10, 5-30, 5-25, 5-20, 5-15, 5-10, 2, 3, 4, 5, 6, 7, 8, 9, 10, 11, 12, 13, 14, 15, 16, 17, 18, 19, or 20) consecutive 2′-F modified sugars. In some embodiments, an oligonucleotide comprises two or more 2′-F blocks as described herein separated by one or more separating blocks as described herein. In some embodiments, a 2′-F block has 2, 3, 4, 5, 6, 7, 8, 9, or 10 2′-F modified sugars. In some embodiments, a 2′-F block has no more than 2, 3, 4, 5, 6, 7, 8, 9, or 10 2′-F modified sugars. In some embodiments, each sugar in each 2′-F blocks is a 2′-F modified sugar, and each 2′-F block independently has 2, 3, 4, 5, 6, 7, 8, 9, or 10 2′-F modified sugars. In some embodiments, each sugar in each 2′-F blocks is a 2′-F modified sugar, and each 2′-F block independently has no more than 2, 3, 4, 5, 6, 7, 8, 9, or 10 2′-F modified sugars. In some embodiments, each sugar in each 2′-F blocks is a 2′-F modified sugar, and each 2′-F block independently has no more than 10 2′-F modified sugars. In some embodiments, each sugar in each 2′-F blocks is a 2′-F modified sugar, and each 2′-F block independently has no more than 9 2′-F modified sugars. In some embodiments, each sugar in each 2′-F blocks is a 2′-F modified sugar, and each 2′-F block independently has no more than 8 2′-F modified sugars. In some embodiments, each sugar in each 2′-F blocks is a 2′-F modified sugar, and each 2′-F block independently has no more than 7 2′-F modified sugars. In some embodiments, each sugar in each 2′-F blocks is a 2′-F modified sugar, and each 2′-F block independently has no more than 6 2′-F modified sugars. In some embodiments, each sugar in each 2′-F blocks is a 2′-F modified sugar, and each 2′-F block independently has no more than 5 2′-F modified sugars. In some embodiments, each sugar in each 2′-F blocks is a 2′-F modified sugar, and each 2′-F block independently has no more than 4 2′-F modified sugars. In some embodiments, each block bonded to a 2′-F block is independently a block that comprises no 2′-F modified sugar. In some embodiments, each block bonded to a 2′-F block is independently a block that comprises a natural DNA or RNA sugar, a 2′-OR modified sugar wherein R is optionally substituted C₁₋₆ aliphatic or a bicyclic sugar. In some embodiments, each block bonded to a 2′-F block is independently a block that comprises a natural DNA or RNA sugar, a 2′-OMe modified sugar, 2′-MOE modified sugar or a bicyclic sugar. In some embodiments, each block bonded to a 2′-F block is independently a block that comprises a natural DNA or RNA sugar, a 2′-OMe modified sugar or 2′-MOE modified sugar. In some embodiments, each nucleoside in a first domain bonded to a 2′-F block in a first domain is independently a 2′-OR modified sugar wherein R is optionally substituted C₁₋₆ aliphatic or a bicyclic sugar. In some embodiments, each nucleoside in a first domain bonded to a 2′-F block in a first domain is independently a 2′-OR modified sugar wherein R is optionally substituted C₁₋₆ aliphatic. In some embodiments, each nucleoside in a first domain bonded to a 2′-F block in a first domain is independently a 2′-OMe or 2′-MOE modified sugar. In some embodiments, each nucleoside in a second domain bonded to a 2′-F block in a second domain is independently a 2′-OR modified sugar wherein R is optionally substituted C₁₋₆ aliphatic or a bicyclic sugar. In some embodiments, each nucleoside in a second domain bonded to a 2′-F block in a second domain is independently a 2′-OR modified sugar wherein R is optionally substituted C₁₋₆ aliphatic. In some embodiments, each nucleoside in a second domain bonded to a 2′-F block in a second domain is independently a 2′-OMe or 2′-MOE modified sugar.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-OR modified sugars, wherein R is optionally substituted C₁₋₆ aliphatic. In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-OMe or 2′-MOE modified sugars. In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-OMe modified sugars. In some embodiments, a percentage is about or at least about 30%. In some embodiments, a percentage is about or at least about 40%. In some embodiments, a percentage is about or at least about 50%. In some embodiments, a percentage is about or at least about 60%. In some embodiments, a percentage is about or at least about 70%. In some embodiments, a percentage is about or at least about 80%. In some embodiments, a percentage is about or at least about 90%. In some embodiments, a percentage is about or at least about 95%.

In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-OR modified sugars, wherein R is optionally substituted C₁₆ aliphatic. In some embodiments, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars are 2′-MOE modified sugars.

In some embodiments, sugars of the first (5′-end) one or several (e.g., 1, 2, 3, 4, 5, 6, 7, 8, 9, 10, or 1-10, 1-9, 1-8, 1-7, 1-6, 1-5, 1-4, 1-3, 2-10, 2-9, 2-8, 2-7, 2-6, 2-5, 2-4, 2-3, 3-10, 3-9, 3-8, 3-7, 3-6, 3-5, 3-4, etc.) and/or the last (3′-end) one or several (e.g., 1, 2, 3, 4, 5, 6, 7, 8, 9, 10, or 1-10, 1-9, 1-8, 1-7, 1-6, 1-5, 1-4, 1-3, 2-10, 2-9, 2-8, 2-7, 2-6, 2-5, 2-4, 2-3, 3-10, 3-9, 3-8, 3-7, 3-6, 3-5, 3-4, etc.) nucleosides are independently modified sugars. In some embodiments, the first one or several sugars are independently modified sugars. In some embodiments, the last one or several sugars are independently modified sugars. In some embodiments, both the first and last one or several sugars are independently modified sugars. In some embodiments, modified sugars are independently non-2′-F modified sugars, e.g., bicyclic sugars, 2′-OR modified sugars wherein R is as described herein and is not —H (e.g., optionally substituted C₁₋₆ aliphatic). In some embodiments, they are independently selected from bicyclic sugars and 2′-OR modified sugars wherein R is optionally substituted C₁-₆ aliphatic. In some embodiments, they are independently 2′-OR modified sugars wherein R is optionally substituted C₁₋₆ aliphatic. In some embodiments, they are independently 2′-OMe modified sugars and 2′-MOE modified sugars. In some embodiments, the first several sugars comprises one or more 2′-OR modified sugars wherein R is optionally substituted C₁₋₆ aliphatic or bicyclic sugars (e.g., LNA, cEt, etc.) as described herein. In some embodiments, the first several sugars comprises one or more 2′-OR modified sugars wherein R is optionally substituted C₁₋₆ aliphatic. In some embodiments, the first several sugars comprises one or more 2′-OMe modified sugars. In some embodiments, the first several sugars comprises one or more 2′-MOE modified sugars. In some embodiments, the first several sugars comprises one or more 2′-OMe modified sugars and one or more 2′-MOE modified sugars. In some embodiments, the last several sugars comprises one or more 2′-OR modified sugars wherein R is optionally substituted C₁₋₆ aliphatic or bicyclic sugars (e.g., LNA, cEt, etc.) as described herein. In some embodiments, the last several sugars comprises one or more 2′-OR modified sugars wherein R is optionally substituted C₁₋₆ aliphatic. In some embodiments, the last several sugars comprises one or more 2′-OMe modified sugars. In some embodiments, the last several sugars comprises one or more 2′-MOE modified sugars. In some embodiments, the last several sugars comprises one or more 2′-OMe modified sugars and one or more 2′-MOE modified sugars. In some embodiments, the last several sugars are independently 2′-OMe modified sugars. In some embodiments, the first several sugars comprise two or more (e.g., 2, 3, 4, 5, 6, 7, 8, 9, or 10, etc.) consecutive bicyclic sugars or 2′-OR modified sugars wherein R is optionally substituted C₁₋₆ aliphatic. In some embodiments, the first several sugars comprise two or more (e.g., 2, 3, 4, 5, 6, 7, 8, 9, or 10, etc.) consecutive 2′-OR modified sugars wherein R is optionally substituted C₁₋₆ aliphatic. In some embodiments, the first several sugars comprise two or more (e.g., 2, 3, 4, 5, 6, 7, 8, 9, or 10, etc.) consecutive modified sugars wherein each modified sugar is independently a 2′-OMe modified sugar or a 2′-MOE modified sugar. In some embodiments, the first several sugars comprise two or more (e.g., 2, 3, 4, 5, 6, 7, 8, 9, or 10, etc.) consecutive 2′-OMe modified sugars. In some embodiments, the first several sugars comprise two or more (e.g., 2, 3, 4, 5, 6, 7, 8, 9, or 10, etc.) consecutive 2′-MOE modified sugars. In some embodiments, the last several sugars comprise two or more (e.g., 2, 3, 4, 5, 6, 7, 8, 9, or 10, etc.) consecutive 2′-OR modified sugars wherein R is optionally substituted C₁₋₆ aliphatic. In some embodiments, the last several sugars comprise two or more (e.g., 2, 3, 4, 5, 6, 7, 8, 9, or 10, etc.) consecutive modified sugars wherein each modified sugar is independently a 2′-OMe modified sugar or a 2′-MOE modified sugar. In some embodiments, the last several sugars comprise two or more (e.g., 2, 3, 4, 5, 6, 7, 8, 9, or 10, etc.) consecutive 2′-OMe modified sugars. In some embodiments, the last several sugars comprise three or more consecutive 2′-OMe modified sugars. In some embodiments, the last several sugars comprise four or more consecutive 2′-OMe modified sugars. In some embodiments, the last several sugars comprise five or more consecutive 2′-OMe modified sugars. In some embodiments, the last several sugars comprise six or more consecutive 2′-OMe modified sugars. In some embodiments, the last several sugars comprise two or more (e.g., 2, 3, 4, 5, 6, 7, 8, 9, or 10, etc.) consecutive 2′-MOE modified sugars.

In some embodiments, one or more (1, 2, 3, 4, 5, 6, 7, 8, 9, or 10) of the first several (1, 2, 3, 4, 5, 6, 7, 8, 9, or 10) sugars are modified sugars. In some embodiments, one or more (1, 2, 3, 4, 5, 6, 7, 8, 9, or 10) of the first several (1, 2, 3, 4, 5, 6, 7, 8, 9, or 10) sugars are modified sugars each independently selected from a 2′-OR modified sugar wherein R is optionally substituted C₁₋₆ aliphatic and a bicyclic sugar (e.g., a sugar comprising 2′-O—CH₂-4′, wherein the —CH₂— is optionally substituted (e.g., a LNA sugar, a cET sugar (e.g., (S)-cEt))). In some embodiments, two or more of the first several sugars are modified sugars each independently selected from a 2′-OR modified sugar wherein R is optionally substituted C₁₋₆ aliphatic and a bicyclic sugar. In some embodiments, three or more of the first several sugars are modified sugars each independently selected from a 2′-OR modified sugar wherein R is optionally substituted C₁₋₆ aliphatic and a bicyclic sugar. In some embodiments, four or more of the first several sugars are modified sugars each independently selected from a 2′-OR modified sugar wherein R is optionally substituted C₁₋₆ aliphatic and a bicyclic sugar. In some embodiments, the one or more sugars are consecutive. In some embodiments, the first one, two, three or four sugars are modified sugars. In some embodiments, the first two sugars are modified sugars each independently selected from a 2′-OR modified sugar wherein R is optionally substituted C₁0.6 aliphatic and a bicyclic sugar. In some embodiments, the first three sugars are modified sugars each independently selected from a 2′-OR modified sugar wherein R is optionally substituted C₁₋₆ aliphatic and a bicyclic sugar. In some embodiments, the first four sugars are modified sugars each independently selected from a 2′-OR modified sugar wherein R is optionally substituted C₁₋₆ aliphatic and a bicyclic sugar. In some embodiments, each 2′-OR modified sugar is independently a 2′-OMe or 2′-MOE modified sugar. In some embodiments, each bicyclic sugar is independently a LNA sugar or a cEt sugar. In some embodiments, each of the one or more (e.g., 1, 2, 3, 4, or 5) sugars of the first several sugars, or the first several (e.g., 1, 2, 3, 4, or 5) sugar(s), is independently a 2′-OR modified sugar wherein R is optionally substituted C₁₋₆ aliphatic. In some embodiments, each of the one or more (e.g., 1, 2, 3, 4, or 5) sugars of the first several sugars, or the first several (e.g., 1, 2, 3, 4, or 5) sugar(s), is independently a 2′-OMe or 2′-MOE modified sugar. In some embodiments, each of the one or more (e.g., 1, 2, 3, 4, or 5) sugars of the first several sugars, or the first several (e.g., 1, 2, 3, 4, or 5) sugar(s), is independently a 2′-OMe modified sugar. In some embodiments, each of the one or more (e.g., 1, 2, 3, 4, or 5) sugars of the first several sugars, or the first several (e.g., 1, 2, 3, 4, or 5) sugar(s), is independently a 2′-MOE modified sugar. In some embodiments, the first one, two, three, four or more sugars are independently 2′-OMe modified sugars. In some embodiments, the first sugar is a 2′-OMe modified sugar. In some embodiments, the first two sugars are independently 2′-OMe modified sugars. In some embodiments, the first three sugars are independently 2′-OMe modified sugars. In some embodiments, the first four sugars are independently 2′-OMe modified sugars. In some embodiments, the first one, two, three, four or more sugars are independently 2′-MOE modified sugars. In some embodiments, the first sugar is a 2′-MOE modified sugar. In some embodiments, the first two sugars are independently 2′-MOE modified sugars. In some embodiments, the first three sugars are independently 2′-MOE modified sugars. In some embodiments, the first four sugars are independently 2′-MOE modified sugars. In some embodiments, each of such modified sugars is independently the sugar of a nucleoside whose nucleobase is optionally substituted or protected A, T, C, G, or U, or an optionally substituted or protected tautomer of A, T, C, G, or U. In some embodiments, one or more such sugars are independently bonded to a PN linkage. In some embodiments, one or more such sugars are independently bonded to a non-negatively charged internucleotidic linkage. In some embodiments, one or more such sugars are independently bonded to a neutral internucleotidic linkage such as n001. In some embodiments, a non-negatively charged internucleotidic linkage or neutral internucleotidic linkage, e.g., n001, is chirally controlled. In some embodiments, it is Rp. In some embodiments, one or more such sugars are independently bonded to a PS linkage. In some embodiments, one or more such sugars are independently bonded to a phosphorothioate internucleotidic linkage. In some embodiments, a PS linkage, e.g., a phosphorothioate internucleotidic linkage is chirally controlled. In some embodiments, it is Sp. In some embodiments, as described herein, the internucleotidic linkage between the first and second nucleosides is a non-negatively charged internucleotidic linkage. In some embodiments, it is a neutral internucleotidic linkage. In some embodiments, it is a PN linkage. In some embodiments, it is a phosphoryl guanidine internucleotidic linkage. In some embodiments, it is n001. In some embodiments, it is chirally controlled. In some embodiments, it is Rp. In some embodiments, except the internucleotidic linkage between the first and second nucleosides, each internucleotidic linkages bonded to nucleosides comprising the one or more of the first several, or the first several modified sugars are independently PS linkages, e.g., phosphorothioate internucleotidic linkages. In some embodiments, each is chirally controlled. In some embodiments, each is Sp. In some embodiments, a first nucleoside is connected to an additional moiety, e.g., Mod001, optionally through a linker, e.g., L001, through its 5′-end carbon (in some embodiments, via a phosphate group). In some embodiments, the first several is the first 3, 4, 5, 6, etc.

In some embodiments, one or more (1, 2, 3, 4, 5, 6, 7, 8, 9, or 10) of the last several (1, 2, 3, 4, 5, 6, 7, 8, 9, or 10) sugars are modified sugars. In some embodiments, one or more (1, 2, 3, 4, 5, 6, 7, 8, 9, or 10) of the last several (1, 2, 3, 4, 5, 6, 7, 8, 9, or 10) sugars are modified sugars each independently selected from a 2′-OR modified sugar wherein R is optionally substituted C₁₋₆ aliphatic and a bicyclic sugar (e.g., a sugar comprising 2′-O—CH₂-4′, wherein the —CH₂— is optionally substituted (e.g., a LNA sugar, a cET sugar (e.g., (S)-cEt))). In some embodiments, two or more of the last several sugars are modified sugars each independently selected from a 2′-OR modified sugar wherein R is optionally substituted C₁₋₆ aliphatic and a bicyclic sugar. In some embodiments, three or more of the last several sugars are modified sugars each independently selected from a 2′-OR modified sugar wherein R is optionally substituted C₁₋₆ aliphatic and a bicyclic sugar. In some embodiments, four or more of the last several sugars are modified sugars each independently selected from a 2′-OR modified sugar wherein R is optionally substituted C₁₋₆ aliphatic and a bicyclic sugar. In some embodiments, the one or more sugars are consecutive. In some embodiments, the last one, two, three or four sugars are modified sugars. In some embodiments, the last two sugars are modified sugars each independently selected from a 2′-OR modified sugar wherein R is optionally substituted C₁₋₆ aliphatic and a bicyclic sugar. In some embodiments, the last three sugars are modified sugars each independently selected from a 2′-OR modified sugar wherein R is optionally substituted C₁₋₆ aliphatic and a bicyclic sugar. In some embodiments, the last four sugars are modified sugars each independently selected from a 2′-OR modified sugar wherein R is optionally substituted C₁₋₆ aliphatic and a bicyclic sugar. In some embodiments, each 2′-OR modified sugar is independently a 2′-OMe or 2′-MOE modified sugar. In some embodiments, each bicyclic sugar is independently a LNA sugar or a cEt sugar. In some embodiments, each of the one or more (e.g., 1, 2, 3, 4, or 5) sugars of the last several sugars, or the last several (e.g., 1, 2, 3, 4, or 5) sugar(s), is independently a 2′-OR modified sugar wherein R is optionally substituted C₁₋₆ aliphatic. In some embodiments, each of the one or more (e.g., 1, 2, 3, 4, or 5) sugars of the last several sugars, or the last several (e.g., 1, 2, 3, 4, or 5) sugar(s), is independently a 2′-OMe or 2′-MOE modified sugar. In some embodiments, each of the one or more (e.g., 1, 2, 3, 4, or 5) sugars of the last several sugars, or the last several (e.g., 1, 2, 3, 4, or 5) sugar(s), is independently a 2′-OMe modified sugar. In some embodiments, each of the one or more (e.g., 1, 2, 3, 4, or 5) sugars of the last several sugars, or the last several (e.g., 1, 2, 3, 4, or 5) sugar(s), is independently a 2′-MOE modified sugar. In some embodiments, the last one, two, three, four or more sugars are independently 2′-OMe modified sugars. In some embodiments, the last sugar is a 2′-OMe modified sugar. In some embodiments, the last two sugars are independently 2′-OMe modified sugars. In some embodiments, the last three sugars are independently 2′-OMe modified sugars. In some embodiments, the last four sugars are independently 2′-OMe modified sugars. In some embodiments, the last one, two, three, four or more sugars are independently 2′-MOE modified sugars. In some embodiments, the last sugar is a 2′-MOE modified sugar. In some embodiments, the last two sugars are independently 2′-MOE modified sugars. In some embodiments, the last three sugars are independently 2′-MOE modified sugars. In some embodiments, the last four sugars are independently 2′-MOE modified sugars. In some embodiments, each of such modified sugars is independently the sugar of a nucleoside whose nucleobase is optionally substituted or protected A, T, C, G, or U, or an optionally substituted or protected tautomer of A, T, C, G, or U. In some embodiments, one or more such sugars are independently bonded to a PN linkage. In some embodiments, one or more such sugars are independently bonded to a non-negatively charged internucleotidic linkage. In some embodiments, one or more such sugars are independently bonded to a neutral internucleotidic linkage such as n001. In some embodiments, a non-negatively charged internucleotidic linkage or neutral internucleotidic linkage, e.g., n001, is chirally controlled. In some embodiments, it is Rp. In some embodiments, one or more such sugars are independently bonded to a PS linkage. In some embodiments, one or more such sugars are independently bonded to a phosphorothioate internucleotidic linkage. In some embodiments, a PS linkage is chirally controlled. In some embodiments, a phosphorothioate internucleotidic linkage is chirally controlled. In some embodiments, it is Sp. In some embodiments, as described herein, the internucleotidic linkage between the last and second last nucleosides is a non-negatively charged internucleotidic linkage. In some embodiments, it is a neutral internucleotidic linkage. In some embodiments, it is a PN linkage In some embodiments, it is a phosphoryl guanidine internucleotidic linkage. In some embodiments, it is n001. In some embodiments, it is chirally controlled. In some embodiments, it is Rp. In some embodiments, except the internucleotidic linkage between the last and second last nucleosides, each internucleotidic linkages bonded to nucleosides comprising the one or more of the last several, or the last several modified sugars are independently phosphorothioate internucleotidic linkages. In some embodiments, each is chirally controlled. In some embodiments, each is Sp. In some embodiments, the last several is the last 3, 4, 5, etc.

In some embodiments, a sugar at position +1 is a 2′-F modified sugar. In some embodiments, a sugar at position +1 is a natural DNA sugar. In some embodiments, a sugar at position 0 is a natural DNA sugar (nucleoside at position 0 is opposite to a target adenosine when aligned). In some embodiments, a sugar at position −1 is a DNA sugar. In some embodiments, a sugar at position −2 is a 2′-OR modified sugar wherein R is optionally substituted C₁₋₆ aliphatic or a bicyclic sugar (e.g., a sugar comprising 2′-O—CH₂-4′, wherein the —CH₂— is optionally substituted (e.g., a LNA sugar, a cET sugar (e.g., (S)-cEt))). In some embodiments, it is a 2′-OR modified sugar wherein R is optionally substituted C₁₋₆ aliphatic. In some embodiments, it is a 2′-OMe modified sugar. In some embodiments, it is a 2′-MOE modified sugar. In some embodiments, it is a bicyclic sugar. In some embodiments, it is a LNA sugar. In some embodiments, it is a cEt sugar. In some embodiments, a sugar at position −3 is a 2′-F modified sugar. In some embodiments, each sugar after position −3 (e.g., position −4, −5, −6, etc.) is independently a 2′-OR modified sugar wherein R is optionally substituted C₁₋₆ aliphatic or a bicyclic sugar (e.g., a sugar comprising 2′-O—CH₂-4′, wherein the —CH₂— is optionally substituted (e.g., a LNA sugar, a cET sugar (e.g., (S)-cEt))). In some embodiments, each is independently a 2′-OR modified sugar wherein R is optionally substituted C₁₋₆ aliphatic or a bicyclic sugar. In some embodiments, each is independently a 2′-OMe or 2′-MOE modified sugar. In some embodiments, each is a 2′-OMe modified sugar. In some embodiments, each is a 2′-MOE modified sugar. In some embodiments, one or more are independently 2′-OMe modified sugars, and one or more are independently 2′-MOE modified sugars. In some embodiments, as described herein, the internucleotidic linkage between nucleosides at positions −1 and −2 is a non-negatively charged internucleotidic linkage. In some embodiments, it is a neutral internucleotidic linkage. In some embodiments, it is a phosphoryl guanidine internucleotidic linkage. In some embodiments, it is n001. In some embodiments, it is chirally controlled. In some embodiments, it is Sp. In some embodiments, it is Rp. In some embodiments, the internucleotidic linkage between nucleosides at positions −2 and −3 is a natural phosphate linkage. In some embodiments, as described herein, the internucleotidic linkage between the last and second last nucleosides is a non-negatively charged internucleotidic linkage. In some embodiments, it is a neutral internucleotidic linkage. In some embodiments, it is a phosphoryl guanidine internucleotidic linkage. In some embodiments, it is n001. In some embodiments, it is chirally controlled. In some embodiments, it is Rp. In some embodiments, each internucleotidic linkages between nucleosides to the 3′-side of a nucleoside opposite to a target adenosine, except those between nucleosides at positions −1 and −2, and between nucleosides at positions −2 and −3, and between the last and the second last nucleosides, is independently a phosphorothioate internucleotidic linkages. In some embodiments, each phosphorothioate internucleotidic linkage is chirally controlled. In some embodiments, each is Sp.

In some embodiments, the first and/or last one or several sugars are modified sugars, e.g., bicyclic sugars and/or 2′-OR modified sugars wherein R is optionally substituted C₁₋₆ aliphatic (e.g., 2′-OMe modified sugars, 2′-MOE modified sugars, etc.). In some embodiments, such sugars may increase stability, affinity and/or activity of an oligonucleotide. In some embodiments, when conjugated to one or more additional chemical moieties, sugars at 5′- and/or 3′-ends of oligonucleotides are not bicyclic sugars or 2′-OR modified sugars wherein R is optionally substituted C₁₋₆ aliphatic. In some embodiments, a 5′-end sugar is a bicyclic sugar or a 2′-OR modified sugar wherein R is optionally substituted C₁₋₆ aliphatic. In some embodiments, such a 5′-end sugar is not connected to an additional chemical moiety. In some embodiments, a 5′-end sugar is a 2′-F modified sugar. In some embodiments, a 5′-end sugar is a 2′-F modified sugar conjugated to an additional chemical moiety. In some embodiments, a 3′-end sugar is a bicyclic sugar or a 2′-OR modified sugar wherein R is optionally substituted C₁₋₆ aliphatic. In some embodiments, such a 3′-end sugar is not connected to an additional chemical moiety. In some embodiments, a 3′-end sugar is a 2′-F modified sugar. In some embodiments, a 3′-end sugar is a 2′-F modified sugar conjugated to an additional chemical moiety. In some embodiments, the last several sugars are 3′-side sugars relative to a nucleoside opposite to a target adenosine (e.g., sugars of 3′-side nucleosides such as N₋₁, N₋₂, etc.). In some embodiments, the last several sugars or the 3′-side sugars comprises one or more (e.g., 1, 2, 3, 4, 5, 6, 7, 8, 9, or 10 or more) 2′-F modified sugars. In some embodiments, the last several sugars or the 3′-side sugars comprises two or more (e.g., 2, 3, 4, 5, 6, 7, 8, 9, or 10 or more) consecutive 2′-F modified sugars. In some embodiments, the last several sugars or the 3′-side sugars comprises one or more, or two or more consecutive, 2′-F modified sugars, and sugar of the last nucleoside of an oligonucleotide is a bicyclic sugar or a 2′-OR modified sugar wherein R is optionally substituted C₁₋₆ aliphatic. In some embodiments, as described herein a 2′-OR modified sugar is a 2′-OMe modified sugar or a 2′-MOE modified sugar; in some embodiments, it is a 2′-OMe modified sugar; in some embodiments, it is a 2′-MOE modified sugar. In some embodiments, the last several sugars or the 3′-side sugars comprises one or more, or two or more consecutive, 2′-F modified sugars, and sugar of the last nucleoside of an oligonucleotide is a 2′-OR modified sugar wherein R is optionally substituted C₁₋₆ aliphatic. In some embodiments, the last several sugars or the 3′-side sugars comprises one or more, or two or more consecutive, 2′-F modified sugars, and sugar of the last nucleoside of an oligonucleotide is a 2′-OMe modified sugar or a 2′-MOE modified sugar. In some embodiments, the last several sugars or the 3′-side sugars comprises one or more, or two or more consecutive, 2′-F modified sugars, and sugar of the last nucleoside of an oligonucleotide is a 2′-OMe modified sugar. In some embodiments, the last several sugars or the 3′-side sugars comprises one or more, or two or more consecutive, 2′-F modified sugars, and sugar of the last nucleoside of an oligonucleotide is a 2′-MOE modified sugar. In some embodiments, two and no more than two nucleosides at the 3′-side of a nucleoside opposite to an adenosine independently have a 2′-F modified sugar. In some embodiments, they are at positions −4 and −5. In some embodiments, they are the second and third last nucleosides of an oligonucleotide. In some embodiments, one and no more than one nucleoside at the 3′-side of a nucleoside opposite to an adenosine has a 2′-F modified sugar. In some embodiments, it is at position −3. In some embodiments, it is 4th last nucleoside of an oligonucleotide.

In some embodiments, a bicyclic sugar or a 2′-OR modified sugar wherein R is optionally substituted C₁₋₆ aliphatic is present in a region which comprises one or more (e.g., 1-30, 1-25, 1-20, 1-15, 1-10, 2-30, 2-25, 2-20, 2-25, 2-10, or 1, 2, 3, 4, 5, 6, 7, 8, 9, 10, 11, 12, 13, 14, 15, 16, 17, 18, 19, 20 or more) sugars are 2′-F modified. In some embodiments, a majority of sugars as described herein in such a region are 2′-F modified sugars. In some embodiments, two or more 2′-F modified sugars are consecutive. In some embodiments, a region is a first domain. In some embodiments, a bicyclic sugar is present in such a region. In some embodiments, a 2′-OR modified sugar wherein R is optionally substituted C₁₋₆ aliphatic is present in such a region. In some embodiments, a 2′-OMe modified sugar is present in such a region. In some embodiments, a 2′-MOE modified sugar is present in such a region.

In some embodiments, one or more sugars at positions −5, −4, −3, +1, +2, +4, +5, +6, +7, and +8 (position 0 being the position of a nucleoside opposite to a target adenosine; “+” is going from a nucleoside opposite to a target adenosine toward 5′-end of an oligonucleotide, and “−” is going from a nucleoside opposite to a target adenosine toward 3′-end of an oligonucleotide; for example, in 5′-N₁N₀N₋₁-3′, if No is a nucleoside opposite to a target adenosine, it is at position 0, and N₁ is at position +1 and N₁ is at position −1) are independently 2′-F modified sugars. In some embodiments, a sugar at position +1, and one or more sugars at positions −5, −4, −3, +2, +4, +5, +6, +7, and +8, are independently 2′-F modified sugars. In some embodiments, a sugar at position +1, and one sugar at position −5, −4, −3, +2, +4, +5, +6, +7, and +8, are independently 2′-F modified sugars.

In some embodiments, an oligonucleotide comprises one or more (e.g., 1, 2, 3, 4, 5, 6, 7, 8, 9, 10 or more, 2-10, 3-10, 2-5, 2-4, 2-3, 3-5, 3-4, etc.) natural DNA sugars. In some embodiments, one or more natural DNA sugars are at an editing region, e.g., positions +1, 0, and/or −1. In some embodiments, a natural DNA sugar is within the first several nucleosides of an oligonucleotides (e.g., the first 1, 2, 3, 4, 5, 6, 7, 8, 9, or 10 nucleosides). In some embodiments, the first, second, and/or third nucleosides of an oligonucleotides independently have a natural DNA sugar. In some embodiments, a natural DNA sugar is bonded to a modified internucleotidic linkage such as a non-negatively charged internucleotidic linkage, a neutral internucleotidic linkage, a phosphoryl guanidine internucleotidic linkage, n001, or a phosphorothioate internucleotidic linkage (in various embodiments, Sp).

Oligonucleotides may contain various types of internucleotidic linkages. In some embodiments, oligonucleotides comprises one or more modified internucleotidic linkages. In some embodiments, a modified internucleotidic linkage is a chiral internucleotidic linkages. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral internucleotidic linkage. In some embodiments, a modified internucleotidic linkage is a phosphoryl guanidine internucleotidic linkage. In some embodiments, a modified internucleotidic linkage is n001. In some embodiments, oligonucleotides comprises one or more natural phosphate linkages. In some embodiments, a natural phosphate linkage bonds to a nucleoside comprising a modified sugar that can improve stability (e.g., resistance toward nuclease). In some embodiments, a natural phosphate linkage bonds to a bicyclic sugar. In some embodiments, a natural phosphate linkage bonds to a 2′-modified sugar. In some embodiments, a natural phosphate linkage bonds to a 2′-OR modified sugar, wherein R is optionally substituted C₁₋₆ aliphatic. In some embodiments, a natural phosphate linkage bonds to a 2′-OMe modified sugar. In some embodiments, a natural phosphate linkage bonds to a 2′-MOE modified sugar. In some embodiments, an oligonucleotide comprises a phosphorothioate internucleotidic linkage, a non-negatively charged internucleotidic linkage, and a natural phosphate linkage. In some embodiments, an oligonucleotide comprises a phosphorothioate internucleotidic linkage, a neutral internucleotidic linkage, and a natural phosphate linkage. In some embodiments, an oligonucleotide comprises a phosphorothioate internucleotidic linkage, a phosphoryl guanidine internucleotidic linkage, and a natural phosphate linkage. In some embodiments, an oligonucleotide comprises a phosphorothioate internucleotidic linkage, n001, and a natural phosphate linkage. In some embodiments, each chiral internucleotidic linkage is independently chirally controlled. In some embodiments, one or more chiral internucleotidic linkage is not chirally controlled. In some embodiments, each phosphorothioate internucleotidic linkage is independently chirally controlled. In some embodiments, each chiral internucleotidic linkage is independently chirally controlled. In some embodiments, a majority or each phosphorothioate internucleotidic linkage is Sp as described herein. In some embodiments, a majority or each non-negatively charged internucleotidic linkage, e.g., n001, is Rp. In some embodiments, a majority or each non-negatively charged internucleotidic linkage, e.g., n001, is Sp.

In some embodiments, an oligonucleotide comprises a phosphorothioate internucleotidic linkage and a non-negatively charged internucleotidic linkage. In some embodiments, an oligonucleotide comprises a phosphorothioate internucleotidic linkage and a neutral internucleotidic linkage. In some embodiments, an oligonucleotide comprises a phosphorothioate internucleotidic linkage and a phosphoryl guanidine internucleotidic linkage. In some embodiments, an oligonucleotide comprises a phosphorothioate internucleotidic linkage and n001. In some embodiments, each chiral internucleotidic linkage is independently chirally controlled. In some embodiments, one or more chiral internucleotidic linkage is not chirally controlled. In some embodiments, each phosphorothioate internucleotidic linkage is independently chirally controlled. In some embodiments, each chiral internucleotidic linkage is independently chirally controlled. In some embodiments, a majority or each phosphorothioate internucleotidic linkage is Sp as described herein. In some embodiments, one or more (e.g., 1, 2, 3, 4, or 5) phosphorothioate internucleotidic linkages are Rp. In some embodiments, a majority or each non-negatively charged internucleotidic linkage, e.g., n001, is Rp. In some embodiments, a majority or each non-negatively charged internucleotidic linkage, e.g., n001, is Sp. In some embodiments, an oligonucleotide comprises no natural phosphate linkages. In some embodiments, each internucleotidic linkage is independently a phosphorothioate or a non-negatively charged internucleotidic linkage. In some embodiments, each internucleotidic linkage is independently a phosphorothioate or a neutral charged internucleotidic linkage. In some embodiments, each internucleotidic linkage is independently a phosphorothioate or phosphoryl guanidine internucleotidic linkages. In some embodiments, each internucleotidic linkage is independently a phosphorothioate or n001 internucleotidic linkage. In some embodiments, the last internucleotidic linkage of an oligonucleotide is a non-negatively charged internucleotidic linkage, or is a neutral internucleotidic linkage, or is a phosphoryl guanidine internucleotidic linkage, or is n001.

In some embodiments, oligonucleotides of the present disclosure comprise one or more modified nucleobases. Various modifications can be introduced to a sugar and/or nucleobase in accordance with the present disclosure. For example, in some embodiments, a modification is a modification described in U.S. Pat. No. 9,006,198. In some embodiments, a modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the sugars, bases, and internucleotidic linkages of each of which are independently incorporated herein by reference.

In some embodiments, a nucleobase in a nucleoside is or comprises Ring BA which has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-c, BA-IV, BA-IV-a, BA-IV-b, BA-V, BA-V-a, BA-V-b, or BA-VI, or a tautomer of Ring BA, wherein the nucleobase is optionally substituted or protected.

In some embodiments, a sugar is a modified sugar comprising a 2′-modificatin, e.g., 2′-F, 2′-OR wherein R is optionally substituted aliphatic, or a bicyclic sugar (e.g., a LNA sugar), or a acyclic sugar (e.g., a UNA sugar).

In some embodiments, as described herein, provided oligonucleotides comprise one or more domains, each of which independently has certain lengths, modifications, linkage phosphorus stereochemistry, etc., as described herein. In some embodiments, the present disclosure provides an oligonucleotide comprising one or more modified sugars and/or one or more modified internucleotidic linkages, wherein the oligonucleotide comprises a first domain and a second domain each independently comprising one or more nucleobases. In some embodiments, the present disclosure provides oligonucleotide comprising one or more domains and/or subdomains as described herein. In some embodiments, the present disclosure provides oligonucleotides comprising a first domain as described herein. In some embodiments, the present disclosure provides oligonucleotides comprising a second domain as described herein. In some embodiments, the present disclosure provides oligonucleotides comprising a first subdomain as described herein. In some embodiments, the present disclosure provides oligonucleotides comprising a second subdomain as described herein. In some embodiments, the present disclosure provides oligonucleotides comprising a third subdomain as described herein. In some embodiments, the present disclosure provides oligonucleotides comprising one or more regions each independently selected from a first domain, a second domain, a first subdomain, a second subdomain and a third subdomain, each of which is independently as described herein. In some embodiments, a first domain is connected to a second domain through a linker. In some embodiments, a first domain is directly connected to a linker.

In some embodiments, an oligonucleotide or a portion thereof (e.g., a first domain, a second domain, a first subdomain, a second subdomain, a third subdomain, etc.) comprises a certain level of modified sugars. In some embodiments, a modified sugar comprises a 2′-modification. In some embodiments, a modified sugar is a bicyclic sugar. In some embodiments, a modified sugar is an acyclic sugar (e.g., by breaking a C2—C3 bond of a corresponding cyclic sugar). In some embodiments, a modified sugar comprises a 5′-modification. Typically, oligonucleotides of the present disclosure have a free 5′—OH at its 5′-end and a free 3′—OH at its 3′-end unless indicated otherwise, e.g., by context. In some embodiments, a 5′-end sugar of an oligonucleotide may comprise a modified 5′—OH.

In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, a majority is at least 50%, 60%, 65%, 70%, 75%, 80%, 85%, 90%, 95%, or more. In some embodiments, a majority is about 50%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%. In some embodiments, a majority is about or at least about 50%. In some embodiments, a majority is about or at least about 55%. In some embodiments, a majority is about or at least about 60%. In some embodiments, a majority is about or at least about 65%. In some embodiments, a majority is about or at least about 70%. In some embodiments, a majority is about or at least about 75%. In some embodiments, a majority is about or at least about 80%. In some embodiments, a majority is about or at least about 85%. In some embodiments, a majority is about or at least about 90%. In some embodiments, a majority is about or at least about 95%.

In some embodiments, an oligonucleotide or a portion thereof (e.g., a first domain, a second domain, a first subdomain, a second subdomain, a third subdomain, etc.) comprises a certain level of modified internucleotidic linkages. In some embodiments, an oligonucleotide or a portion thereof (e.g., a first domain, a second domain, a first subdomain, a second subdomain, a third subdomain, etc.) comprises a certain level of chiral internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, an oligonucleotide or a portion thereof (e.g., a first domain, a second domain, a first subdomain, a second subdomain, a third subdomain, etc.) comprises a certain level of chirally controlled internucleotidic linkages. In some embodiments, an oligonucleotide or a portion thereof (e.g., a first domain, a second domain, a first subdomain, a second subdomain, a third subdomain, etc.) comprises a certain level of Sp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, an oligonucleotide or a portion thereof (e.g., a first domain, a second domain, a first subdomain, a second subdomain, a third subdomain, etc.) comprises a certain level of Sp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, about 1-50, 1-40, 1-30, e.g., about 1, 2, 3, 4, 5, 6, 7, 8, 9, 10, 11, 12, 13, 14, 15, 16, 17, 18, 19, or 20 internucleotidic linkages are independently Sp chiral internucleotidic linkages. In many embodiments, a high percentage (e.g., relative to Rp internucleotidic linkages and/or natural phosphate linkages) of Sp internucleotidic linkages in an oligonucleotide or certain portions thereof can provide improved properties and/or activities, e.g., high stability and/or high adenosine editing activity.

In some embodiments, an oligonucleotide or a portion thereof (e.g., a first domain, a second domain, a first subdomain, a second subdomain, a third subdomain, etc.) comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

While not wishing to be bound by theory, it is noted that in some instances Rp and Sp configurations of internucleotidic linkages may affect structural changes in helical conformations of double stranded complexes formed by oligonucleotides and target nucleic acids such as RNA, and ADAR proteins may recognize and interact various targets (e.g., double stranded complexes formed by oligonucleotides and target nucleic acids such as RNA) through multiple domains. In some embodiments, provided oligonucleotides and compositions thereof promote and/or enhance interaction profiles of oligonucleotide, target nucleic acids, and/or ADAR proteins to provide efficient adenosine modification by ADAR proteins through incorporation of various modifications and/or control of stereochemistry.

In some embodiments, an oligonucleotide can have or comprise a base sequence; internucleotidic linkage, base modification, sugar modification, additional chemical moiety, or pattern thereof; and/or any other structural element described herein, e.g., in Tables.

In some embodiments, a provided oligonucleotide or composition is characterized in that, when it is contacted with a target nucleic acid comprising a target adenosine in a system (e.g., an ADAR-mediated deamination system), modification of the target adenosine (e.g., deamination of the target A) is improved relative to that observed under reference conditions (e.g., selected from the group consisting of absence of the composition, presence of a reference oligonucleotide or composition, and combinations thereof). In some embodiments, modification, e.g., ADAR-mediated deamination (e.g., endogenous ADAR-mediated deamination) is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18,19, 20,21,22,23,24,25,26,27,28,29,30,40,50,60,70,80,90, 100, 200, 300, 400, 500, 600, 700, 800, 900, 1000 fold or more.

In some embodiments, oligonucleotides are provided as salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as ammonium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, oligonucleotides are chiral controlled, comprising one or more chirally controlled internucleotidic linkages. In some embodiments, provided oligonucleotides are stereochemically pure. In some embodiments, provided oligonucleotides or compositions thereof are substantially pure of other stereoisomers. In some embodiments, the present disclosure provides chirally controlled oligonucleotide compositions.

As described herein, oligonucleotides of the present disclosure can be provided in high purity (e.g., 50%-100%). In some embodiments, oligonucleotides of the present disclosure are of high stereochemical purity (e.g., 50%-100%). In some embodiments, oligonucleotides in provided compositions are of high stereochemical purity (e.g., high percentage (e.g., 50%-100%) of a stereoisomer compared to the other stereoisomers of the same oligonucleotide). In some embodiments, a percentage is at least or about 50%. In some embodiments, a percentage is at least or about 60%. In some embodiments, a percentage is at least or about 70%. In some embodiments, a percentage is at least or about 75%. In some embodiments, a percentage is at least or about 80%. In some embodiments, a percentage is at least or about 85%. In some embodiments, a percentage is at least or about 90%. In some embodiments, a percentage is at least or about 95%.

### First Domains

As described herein, in some embodiment, an oligonucleotide comprises a first domain and a second domain. In some embodiments, an oligonucleotide consists of a first domain and a second domain. Certain embodiments are described below as examples.

In some embodiments, the length of a first domain is about or at least about 5-100, 5-50, 5-40, 5-30, 5-20, 10-100, 10-50, 10-40, 10-30, 10-20,5,6,7, 8,9, 10, 11, 12, 13, 14, 15, 16, 17, 18, 19, 20, 21, 22, 23, 24, 25, 26, 27, 28, 29 or 30 nucleobases. In some embodiments, a first domain has a length of about 2-50 or more, e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40, or 50 or more nucleobases. In some embodiments, a first domain has a length of about 5-30 nucleobases. In some embodiments, a first domain has a length of about 10-30 nucleobases. In some embodiments, a first domain has a length of about 5-20 nucleobases. In some embodiments, a first domain has a length of about 7-16 nucleobases. In some embodiments, a first domain has a length of 7 nucleobases. In some embodiments, a first domain has a length of 8 nucleobases. In some embodiments, a first domain has a length of 9 nucleobases. In some embodiments, a first domain has a length of 10 nucleobases. In some embodiments, a first domain has a length of 11 nucleobases. In some embodiments, a first domain has a length of 12 nucleobases. In some embodiments, a first domain has a length of 13 nucleobases. In some embodiments, a first domain has a length of 14 nucleobases. In some embodiments, a first domain has a length of 15 nucleobases. In some embodiments, a first domain has a length of 16 nucleobases. In some embodiments, a first domain has a length of 17 nucleobases. In some embodiments, a first domain has a length of 18 nucleobases. In some embodiments, a first domain has a length of 19 nucleobases. In some embodiments, a first domain has a length of 20 nucleobases. In some embodiments, a first domain has a length of 21 or more nucleobases. In some embodiments, a first domain has the same length as a first portion.

In some embodiments, about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases in the first domain are each independently optionally substituted A, T, C, G, or U, or an optionally substituted tautomer of A, T, C, G or U. In some embodiments, each nucleobase in a first domain is independently optionally substituted A, T, C, G, or U, or an optionally substituted tautomer of A, T, C, G or U. In some embodiments, about 10-30 nucleobases in the first domain are each independently optionally substituted A, T, C, G, or U, or an optionally substituted tautomer of A, T, C, G or U. In some embodiments, about 10-25 nucleobases in the first domain are each independently optionally substituted A, T, C, G, or U, or an optionally substituted tautomer of A, T, C, G or U. In some embodiments, about 10 nucleobases in the first domain are each independently optionally substituted A, T, C, G, or U, or an optionally substituted tautomer of A, T, C, G or U. In some embodiments, about 11 nucleobases in the first domain are each independently optionally substituted A, T, C, G, or U, or an optionally substituted tautomer of A, T, C, G or U. In some embodiments, about 12 nucleobases in the first domain are each independently optionally substituted A, T, C, G, or U, or an optionally substituted tautomer of A, T, C, G or U. In some embodiments, about 13 nucleobases in the first domain are each independently optionally substituted A, T, C, G, or U, or an optionally substituted tautomer of A, T, C, G or U. In some embodiments, about 14 nucleobases in the first domain are each independently optionally substituted A, T, C, G, or U, or an optionally substituted tautomer of A, T, C, G or U. In some embodiments, about 15 nucleobases in the first domain are each independently optionally substituted A, T, C, G, or U, or an optionally substituted tautomer of A, T, C, G or U. In some embodiments, about 16 nucleobases in the first domain are each independently optionally substituted A, T, C, G, or U, or an optionally substituted tautomer of A, T, C, G or U. In some embodiments, about 17 nucleobases in the first domain are each independently optionally substituted A, T, C, G, or U, or an optionally substituted tautomer of A, T, C, G or U. In some embodiments, about 18 nucleobases in the first domain are each independently optionally substituted A, T, C, G, or U, or an optionally substituted tautomer of A, T, C, G or U. In some embodiments, about 19 nucleobases in the first domain are each independently optionally substituted A, T, C, G, or U, or an optionally substituted tautomer of A, T, C, G or U. In some embodiments, about 20 nucleobases in the first domain are each independently optionally substituted A, T, C, G, or U, or an optionally substituted tautomer of A, T, C, G or U. In some embodiments, about 21 or more nucleobases in the first domain are each independently optionally substituted A, T, C, G, or U, or an optionally substituted tautomer of A, T, C, G or U. In some embodiments, such nucleobases are each independently optionally substituted A, T, C, G or U. In some embodiments, such nucleobases are each independently A, T, C, G or U.

Base sequences of first domains are typically of sufficient complementarity to first portions so that first domains can form duplexes with first portions of target nucleic acids. In some embodiments, base sequence of a first domain is about 70%-100%, e.g., about or at least about 70%, 75%, 80%, 85%, 90%, 95%, or about 100%, complementary to the base sequence of the first portion. In some embodiments, the complementarity is about 70% or more. In some embodiments, the complementarity is about 75% or more. In some embodiments, the complementarity is about 80% or more. In some embodiments, the complementarity is about 85% or more. In some embodiments, the complementarity is about 90% or more. In some embodiments, the complementarity is about 95% or more. In some embodiments, the complementarity is about 100%. In some embodiments, there are one or more mismatches. In some embodiments, there are one or more wobbles. In some embodiments, there are one or more bulges. In some embodiments, the first nucleobase from the 5′-end of the first domain is complementary to a nucleobase in the first portion. In some embodiments, the first nucleobase from the 3′-end of the first domain is complementary to a nucleobase in the first portion. In some embodiments, when an oligonucleotide is administered or delivered to a system comprising a target nucleic acid or a fragment thereof that comprises a first portion, a first domain can selectively form a duplex with a first portion. In some embodiments, when an oligonucleotide is administered or delivered to a system expressing a target nucleic acid or a fragment thereof that comprises a portion, a first domain can selectively form a duplex with a first portion. In some embodiments, base sequence of a first portion differentiates the first portion from the rest of a target nucleic acid. In some embodiments, base sequence of a first portion differentiates the first portion from the other transcripts in a system. In some embodiments, base sequence of a first portion differentiates a first portion from the other RNA in a system. In some embodiments, base sequence of a first portion differentiates the first portion from the other nucleic acids in a system.

In some embodiments, a first domain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of an oligonucleotide. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a first domain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a first domain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a first domain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a first domain is fully complementary to a target nucleic acid.

In some embodiments, a first domain comprises one or more modified nucleobases.

In some embodiments, a second domain comprises one or more sugars comprising two 2′-H (e.g., natural DNA sugars). In some embodiments, a second domain comprises one or more sugars comprising 2′-OH (e.g., natural RNA sugars). In some embodiments, a first domain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2—C3 bond of a corresponding cyclic sugar).

In some embodiments, a first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars with 2′-F modification. In some embodiments, a first domain comprises about 2-50 (e.g., about 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., 2-40, 2-30, 2-25, 2-20, 2-15, 2-10, 3-40, 3-30, 3-25, 3-20, 3-15, 3-10, 4-40, 4-30, 4-25, 4-20, 4-15, 4-10, 5-40, 5-30, 5-25, 5-20, 5-15, 5-10, 6-40, 6-30, 6-25, 6-20, 6-15, 6-10, 7-40, 7-30, 7-25, 7-20, 7-15, 7-10, 8-40, 8-30, 8-25, 8-20, 8-15, 8-10, 9-40, 9-30, 9-25, 9-20, 9-15, 9-10, 10-40, 10-30, 10-25, 10-20, 10-15, about 2, 3, 4, 5, 6, 7, 8, 9, 10, 11, 12, 13, 14, 15, 16, 17, 18, 19, or 20, etc.) consecutive modified sugars with 2′-F modification. In some embodiments, a first domain comprises 2 consecutive 2′-F modified sugars. In some embodiments, a first domain comprises 3 consecutive 2′-F modified sugars. In some embodiments, a first domain comprises 4 consecutive 2′-F modified sugars. In some embodiments, a first domain comprises 5 consecutive 2′-F modified sugars. In some embodiments, a first domain comprises 6 consecutive 2′-F modified sugars. In some embodiments, a first domain comprises 7 consecutive 2′-F modified sugars. In some embodiments, a first domain comprises 8 consecutive 2′-F modified sugars. In some embodiments, a first domain comprises 9 consecutive 2′-F modified sugars. In some embodiments, a first domain comprises 10 consecutive 2′-F modified sugars. In some embodiments, a first domain comprises two or more 2′-F modified sugar blocks, wherein each sugar in a 2′-F modified sugar block is independently a 2′-F modified sugar. In some embodiments, each 2′-F modified sugar block independently comprises or consists of 2, 3, 4, 5, 6, 7, 8, 9, or 10 consecutive 2′-F modified sugars as described herein. In some embodiments, two consecutive 2′-F modified sugar blocks are independently separated by a separating block which separating block comprises one or more sugars that are independently not 2′-F modified sugars. In some embodiments, each sugar in a separating block is independently not 2′-F modified. In some embodiments, two or more (e.g., 2, 3, 4, 5, 6, 7, 8, 9, or 10 or more) or all sugars in a separating block are independently not 2′-F modified. In some embodiments, a separating block comprises one or more bicyclic sugars (e.g., LNA sugar, cEt sugar, etc.) and/or one or more 2′-OR modified sugars, wherein R is optionally substituted C₁₋₆ aliphatic (e.g., 2′-OMe, 2′-MOE, etc.). In some embodiments, a separating block comprises one or more 2′-OR modified sugars, wherein R is optionally substituted C₁₋₆ aliphatic (e.g., 2′-OMe, 2′-MOE, etc.). In some embodiments, two or more non-2′-F modified sugars are consecutive. In some embodiments, two or more 2′-OR modified sugars wherein R is optionally substituted C₁₋₆ aliphatic (e.g., 2′-OMe, 2′-MOE, etc.) are consecutive. In some embodiments, a separating block comprises two or more (e.g., 2, 3, 4, 5, 6, 7, 8, 9, or 10 or more) 2′-OR modified sugars wherein R is optionally substituted C₁₋₆ aliphatic (e.g., 2′-OMe, 2′-MOE, etc.). In some embodiments, a separating block comprises two or more (e.g., 2, 3, 4, 5, 6, 7, 8, 9, or 10 or more) consecutive 2′-OR modified sugars wherein R is optionally substituted C₁₋₆ aliphatic (e.g., 2′-OMe, 2′-MOE, etc.). In some embodiments, each 2′-OR modified sugar is independently a 2′-OMe or 2′-MOE sugar. In some embodiments, each 2′-OR modified sugar is independently a 2′-OMe sugar. In some embodiments, each 2′-OR modified sugar is independently a 2′-MOE sugar. In some embodiments, a separating block comprises one or more 2′-F modified sugars. In some embodiments, none of 2′-F modified sugars in a separating block are next to each other. In some embodiments, a separating block contain no 2′-F modified sugars. In some embodiments, each sugar in a separating block is independently a 2′-OR modified sugar wherein R is optionally substituted C₁₋₆ aliphatic or a bicyclic sugar. In some embodiments, each sugar in each separating block is independently a 2′-OR modified sugar wherein R is optionally substituted C₁₋₆ aliphatic or a bicyclic sugar. In some embodiments, each sugar in a separating block is independently a 2′-OR modified sugar wherein R is optionally substituted C₁₋₆ aliphatic. In some embodiments, each sugar in each separating block is independently a 2′-OR modified sugar wherein R is optionally substituted C₁₋₆ aliphatic. In some embodiments, each sugar in a separating block is independently a 2′-OMe or 2′-MOE modified sugar. In some embodiments, each sugar in each separating block is independently a 2′-OMe or 2′-MOE modified sugar. In some embodiments, each sugar in a separating block is independently a 2′-OMe modified sugar. In some embodiments, each sugar in a separating block is independently a 2′-MOE modified sugar. In some embodiments, a separating block comprises a 2′-OMe sugar and 2′-MOE modified sugar. In some embodiments, each 2′-F block and each separating block independently contains 1, 2, 3, 4, or 5 nucleosides. In some embodiments, each 2′-F block and each separating block independently contains 1, 2, or 3 nucleosides.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first domain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first domain are independently a 2′-F modified sugar. In some embodiments, a percentage is at least about 40%. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 60%. In some embodiments, a percentage is about or no more than about 70%. In some embodiments, a percentage is about or no more than about 80%. In some embodiments, a percentage is about or no more than about 90%.

In some embodiments, a first domain comprises no bicyclic sugars or 2′-OR modified sugars wherein R is not —H. In some embodiments, a first domain comprises one or more (e.g., 1, 2, 3, 4, 5, 6, 7, 8, 9, or 10) bicyclic sugars and/or 2′-OR modified sugars wherein R is not —H. In some embodiments, a first domain comprises one or more (e.g., 1, 2, 3, 4, 5, 6, 7, 8, 9, or 10) 2′-OR modified sugars wherein R is not —H. In some embodiments, a first domain comprises one or more (e.g., 1, 2, 3, 4, 5, 6, 7, 8, 9, or 10) 2′-OR modified sugars wherein R is optionally substituted C₁₋₁₀ aliphatic. In some embodiments, levels of bicyclic sugars and/or 2′-OR modified sugars wherein R is not —H, individually or combined, are relatively low compared to level of 2′-F modified sugars. In some embodiments, levels of bicyclic sugars and/or 2′-OR modified sugars wherein R is not —H, individually or combined, are about 10%-80% (e.g., about 10%-75%, 10-70%, 10%-65%, 10%-60%, 10%-50%, about 20%-60%, about 30%-60%, about 20%-50%, about 30%-50%, about 5%, 10%, 15%, 20%, 25%, 30%, 35%, 40%, 45%, 50%, 55%, or 60%, etc.). In some embodiments, levels of 2′-OR modified sugars wherein R is not —H combined (e.g., 2′-OMe and 2′-MOE modified sugars combined, if any) are about 10-70% (e.g., about 10%-60%, 10%-50%, about 20%-60%, about 30%-60%, about 20%-50%, about 30-50%, about 5%, 10%, 15%, 20%, 25%, 30%, 35%, 40%, 45%, 50%, 55%, or 60%, etc.). In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first domain comprises 2′-OMe. In some embodiments, no more than about 50% of sugars in a first domain comprises 2′-OMe. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first domain comprises 2′-OR, wherein R is optionally substituted C₁₋₆ aliphatic. In some embodiments, no more than about 50% of sugars in a first domain comprises 2′-OR, wherein R is optionally substituted C₁₋₆ aliphatic. In some embodiments, no more than about 40% of sugars in a first domain comprises 2′-OR, wherein R is optionally substituted C₁₋₆ aliphatic. In some embodiments, no more than about 30% of sugars in a first domain comprises 2′-OR, wherein R is optionally substituted C₁₋₆ aliphatic. In some embodiments, no more than about 25% of sugars in a first domain comprises 2′-OR, wherein R is optionally substituted C₁₋₆ aliphatic. In some embodiments, no more than about 20% of sugars in a first domain comprises 2′-OR, wherein R is optionally substituted C₁₋₆ aliphatic. In some embodiments, no more than about 10% of sugars in a first domain comprises 2′-OR, wherein R is optionally substituted C₁₋₆ aliphatic. In some embodiments, as described herein, 2′-OR is 2′-MOE. In some embodiments, as described herein, 2′-OR is 2′-MOE or 2′-OMe. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars). In some embodiments, a number of 5′-end sugars in a first domain are independently 2′-OR modified sugars, wherein R is not —H. In some embodiments, a number of (e.g., 1, 2, 3, 4, 5, 6, 7, 8, 9, 10 or more) 5′-end sugars in a first domain are independently 2′-OR modified sugars, wherein R is independently optionally substituted C₁₋₆ aliphatic. In some embodiments, the first about 1-10, e.g., 1, 2, 3, 4, 5, 6, 7, 8, 9, or 10, sugars from the 5′-end of a first domain are independently 2′-OR modified sugars, wherein R is independently optionally substituted C₁₋₆ aliphatic. In some embodiments, the first one is 2′-OR modified. In some embodiments, the first two are independently 2′-OR modified. In some embodiments, the first three are independently 2′-OR modified. In some embodiments, the first four are independently 2′-OR modified. In some embodiments, the first five are independently 2′-OR modified. In some embodiments, all 2′-OR modification in a domain (e.g., a first domain), a subdomain (e.g., a first subdomain), or an oligonucleotide are the same. In some embodiments, 2′-OR is 2′-MOE. In some embodiments, 2′-OR is 2′-OMe.

In some embodiments, no sugar in a first domain comprises 2′-OR. In some embodiments, no sugar in a first domain comprises 2′-OMe. In some embodiments, no sugar in a first domain comprises 2′-MOE. In some embodiments, no sugar in a first domain comprises 2′-MOE or 2′-OMe. In some embodiments, no sugar in a first domain comprises 2′-OR, wherein R is optionally substituted C₁₆ aliphatic. In some embodiments, each sugar in a first domain comprises 2′-F.

In some embodiments, about 40-70% (e.g., about 40%-70%, 40%-60%, 50%-70%, 50%-60%, etc., or about 40%, 45%, 50%, 55%, 60%, 65%, 70%, etc.) of sugars in a first domain are 2′-F modified, and about 10%-60% (e.g., about 10%-50%, 20%-60%, 30%-60%, 30%-50%, 40%-50%, etc., or about 10%, 15%, 20%, 25%, 30%, 35%, 40%, 45%, 50%, 55%, or 60%) of sugars in a first domain are independently 2′-OR modified wherein R is not —H or bicyclic sugars (e.g., LNA sugars, cEt sugars, etc.). In some embodiments, about 20%-60% of sugars in a first domain are 2′-F modified. In some embodiments, about 25%-60% of sugars in a first domain are 2′-F modified. In some embodiments, about 30%-60% of sugars in a first domain are 2′-F modified. In some embodiments, about 35%-60% of sugars in a first domain are 2′-F modified. In some embodiments, about 40%-60% of sugars in a first domain are 2′-F modified. In some embodiments, about 50%-60% of sugars in a first domain are 2′-F modified. In some embodiments, about 50%-70% of sugars in a first domain are 2′-F modified. In some embodiments, about 20%-60% of sugars in a first domain are independently 2′-OR modified wherein R is not —H or bicyclic sugars. In some embodiments, about 30%-60% of sugars in a first domain are independently 2′-OR modified wherein R is not —H or bicyclic sugars. In some embodiments, about 40%-60% of sugars in a first domain are independently 2′-OR modified wherein R is not —H or bicyclic sugars. In some embodiments, about 30%-50% of sugars in a first domain are independently 2′-OR modified wherein R is not —H or bicyclic sugars. In some embodiments, about 40%-50% of sugars in a first domain are independently 2′-OR modified wherein R is not —H or bicyclic sugars. In some embodiments, each of the sugars in a first domain that are independently 2′-OR modified wherein R is not —H or bicyclic sugars is independently a 2′-OR modified sugar wherein R is not —H. In some embodiments, each of them is independently a 2′-OR modified sugar wherein R is C₁₋₆ aliphatic. In some embodiments, each of them is independently a 2′-OR modified sugar wherein R is C₁₋₆ alky. In some embodiments, each of them is independently a 2′-OMe or 2′-MOE modified sugar.

In some embodiments, a first domain is to the 5′-side of a second domain, and the first one or more sugars (e.g., 1-5, 1-3, 1, 2, 3, 4, 5, etc.) from the 5′-end of a first domain are each independently a 2′-OR modified sugar or a bicyclic sugar, wherein R is not —H (e.g., optionally substituted C₁₋₆ alkyl). In some embodiments, a first domain is to the 5′-side of a second domain, and the first three sugars from the 5′-end of a first domain are each independently a 2′-OR modified sugar or a bicyclic sugar, wherein R is not —H (e.g., optionally substituted C₁₋₆ alkyl). In some embodiments, each such sugar is independently a 2′-OMe or 2′-MOE modified sugar. In some embodiments, each such sugar is independently a 2′-OMe modified sugar. In some embodiments, each such sugar is independently a 2′-MOE modified sugar.

In some embodiments, a first domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%0, 30%, 40%, 50%, 60%, 65%, 70%, 75%, 80%, 85%, 90%, 95%, or 100%, etc.) of internucleotidic linkages in a first domain are modified internucleotidic linkages. In some embodiments, each internucleotidic linkage in a first domain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first domain is chirally controlled. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first domain is Sp. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a first domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first domain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkages linking two first domain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a first domain is bonded to two nucleosides of the first domain. In some embodiments, an internucleotidic linkage bonded to a nucleoside in a first domain and a nucleoside in a second domain may be properly considered an internucleotidic linkage of a first domain. In some embodiments, an internucleotidic linkage bonded to a nucleoside in a first domain and a nucleoside in a second domain is a modified internucleotidic linkage; in some embodiments, it is a chiral internucleotidic linkage; in some embodiments, it is chirally controlled; in some embodiments, it is Rp; in some embodiments, it is Sp. In many embodiments, a high percentage (e.g., relative to Rp internucleotidic linkages and/or natural phosphate linkages) of Sp internucleotidic linkages provide improved properties and/or activities, e.g., high stability and/or high adenosine editing activity.

In some embodiments, a first domain comprises a certain level of Rp internucleotidic linkages. In some embodiments, a level is about e.g., about 5%-100%, about 10%-100%, 20-100%, 30%-100%,40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90-90%-100, 100%, 20%, 30%, 40%, 50%, 60%, 65%, 70%, 75%, 80%, 85%, 90%, 95%, or 100%, etc. of all internucleotidic linkages in a first domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a first domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a first domain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a first domain is independently chirally controlled. In some embodiments, each is independently Sp or Rp. In some embodiments, a high level is Sp as described herein. In some embodiments, each phosphorothioate internucleotidic linkage in a first domain is chirally controlled and is Sp.

In some embodiments, as illustrated in certain examples, a first domain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a first domain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a first domain are consecutive (e.g., 3 consecutive non-negatively charged internucleotidic linkages). In some embodiments, a non-negatively charged internucleotidic linkage, or two or more consecutive non-negatively charged internucleotidic linkages, are at the 5′-end of a first domain. In some embodiments, the internucleotidic linkage linking the last two nucleosides of a first domain is a non-negatively charged internucleotidic linkage. In some embodiments, the internucleotidic linkage linking the last two nucleosides of a first domain is a Sp non-negatively charged internucleotidic linkage. In some embodiments, the internucleotidic linkage linking the last two nucleosides of a first domain is a Rp non-negatively charged internucleotidic linkage. In some embodiments, the internucleotidic linkage linking the last two nucleosides of a first domain is a phosphorothioate internucleotidic linkage. In some embodiments, the internucleotidic linkage linking the last two nucleosides of a first domain is a Sp phosphorothioate internucleotidic linkage. In some embodiments, the internucleotidic linkage linking the first two nucleosides of a first domain is a non-negatively charged internucleotidic linkage. In some embodiments, the internucleotidic linkage linking the first two nucleosides of a first domain is a Sp non-negatively charged internucleotidic linkage. In some embodiments, the internucleotidic linkage linking the first two nucleosides of a first domain is a Rp non-negatively charged internucleotidic linkage. In some embodiments, the internucleotidic linkage linking the first two nucleosides of a first domain is a phosphorothioate internucleotidic linkage. In some embodiments, the internucleotidic linkage linking the first two nucleosides of a first domain is a Sp phosphorothioate internucleotidic linkage. In some embodiments, a non-negatively charged internucleotidic linkage is a neutral internucleotidic linkage such as n001. In some embodiments, the first two nucleosides of a first domain are the first two nucleosides of an oligonucleotide.

In some embodiments, one or more (e.g., about or at least about 1, 2, 3, 4, 5, 6, 7, 8, 9, 10, 11, 12, 13, 14, 15, 16, 17, 18, 19, or 20, or about or at least about 10%, 20%, 25%, 30%, 35%, 40%, 50%, 60%, 70%, 75%, 80%, 85%, 90% or 95% of all internucleotidic linkage in a first domain) chiral internucleotidic linkage in a first domain are not chirally controlled. In some embodiments, all chiral internucleotidic linkages in a first domain are not chirally controlled. In some embodiments, a first domain comprises one or more blocks of chirally controlled internucleotidic linkages and one or more blocks of non-chirally controlled internucleotidic linkages. In some embodiments, in each block of chirally controlled internucleotidic linkages there are about 1-20, e.g., about 1, 2, 3, 4, 5, 6, 7, 8, 9, 10, 11, 12, 13, 14, 15, 16, 17, 18, 19 or 20 chiral internucleotidic linkages, and each is independently chirally controlled. In some embodiments, in each block of non-chirally controlled internucleotidic linkages there are about 1-20, e.g., about 1, 2, 3, 4, 5, 6, 7, 8, 9, 10, 11, 12, 13, 14, 15, 16, 17, 18, 19 or 20 chiral internucleotidic linkages, and each is independently non-chirally controlled.

In some embodiments, a first domain comprises one or more natural phosphate linkages. In some embodiments, a first domain contains no natural phosphate linkages. In some embodiments, one or more 2′-OR modified sugars wherein R is not —H are independently bonded to a natural phosphate linkage. In some embodiments, one or more 2′-OR modified sugars wherein R is optionally substituted C₁₋₆ aliphatic are independently bonded to a natural phosphate linkage. In some embodiments, one or more 2′-OMe modified sugars are independently bonded to a natural phosphate linkage. In some embodiments, one or more 2′-MOE modified sugars are independently bonded to a natural phosphate linkage. In some embodiments, each 2′-MOE modified sugar is independently bonded to a natural phosphate linkage. In some embodiments, 50% or more (e.g., 50%-100%, 50%-90%, 50-80%, or about 50%, 60%, 66%, 70%, 75%, 80%, 90% or more) 2′-OR modified sugars wherein R is not —H are independently bonded to a natural phosphate linkage. In some embodiments, 50% or more (e.g., 50%-100%, 50%-90%, 50-80%, or about 50%, 60%, 66%, 70%, 75%, 80%, 90% or more) 2′-OMe modified sugars are independently bonded to a natural phosphate linkage. In some embodiments, 50% or more (e.g., 50%-100%, 50%-90%, 50-80%, or about 50%, 60%, 66%, 70%, 75%, 80%, 90% or more) 2′-MOE modified sugars are independently bonded to a natural phosphate linkage. In some embodiments, 50% or more (e.g., 50%-100%, 50%-90%, 50-80%, or about 50%, 60%, 66%, 70%, 75%, 80%, 90% or more) internucleotidic linkages bonded to two 2′-OR modified sugars are independently natural phosphate linkages. In some embodiments, 50% or more (e.g., 50%-100%, 50%-90%, 50-80%, or about 50%, 60%, 66%, 70%, 75%, 80%, 90% or more) internucleotidic linkages bonded to two 2′-OMe or 2′-MOE modified sugars are independently natural phosphate linkages.

In some embodiments, in an oligonucleotide of the present disclosure or a portion thereof, e.g., a first domain, a second domain, a first subdomain, a second subdomain, a third subdomain, etc., each internucleotidic linkage bonded to two 2′-F modified sugars is independently a modified internucleotidic linkage. In some embodiments, it is independently a phosphorothioate internucleotidic linkage or a non-negatively charged internucleotidic linkage such as a phosphoryl guanidine internucleotidic linkage like n001. In some embodiments, it is independently a Sp phosphorothioate internucleotidic linkage or a non-negatively charged internucleotidic linkage such as a phosphoryl guanidine internucleotidic linkage like n001. In some embodiments, it is independently a Sp phosphorothioate internucleotidic linkage or a Rp phosphoryl guanidine internucleotidic linkage like Rp n001. In some embodiments, each phosphorothioate internucleotidic linkage bonded to two 2′-F modified sugars is independently Sp.

In some embodiments, a first domain recruits, promotes or contribute to recruitment of, a protein such as an ADAR protein (e.g., ADAR1, ADAR2, etc.). In some embodiments, a first domain recruits, or promotes or contribute to interactions with, a protein such as an ADAR protein. In some embodiments, a first domain contacts with a RNA binding domain (RBD) of ADAR. In some embodiments, a first domain does not substantially contact with a second RBD domain of ADAR. In some embodiments, a first domain does not substantially contact with a catalytic domain of ADAR which has a deaminase activity. In some embodiments, various nucleobases, sugars and/or internucleotidic linkages may interact with one or more residues of proteins, e.g., ADAR proteins.

### Second Domains

As described herein, in some embodiment, an oligonucleotide comprises a first domain and a second domain. In some embodiments, an oligonucleotide consists of a first domain and a second domain. Certain embodiments of a second domain are described below as examples. In some embodiments, a second domain comprise a nucleoside opposite to a target adenosine to be modified (e.g., conversion to I).

In some embodiments, the length of a second domain is about or at least about 5-100, 5-50, 5-40, 5-30, 5-20, 10-100, 10-50, 10-40, 10-30, 10-20,5, 6,7, 8, 9, 10, 11, 12, 13, 14, 15, 16, 17, 18, 19, 20, 21, 22, 23, 24, 25, 26, 27, 28, 29 or 30 nucleobases. In some embodiments, a second domain has a length of about 2-50 or more, e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40, or 50 or more nucleobases. In some embodiments, a second domain has a length of about 5-30 nucleobases. In some embodiments, a second domain has a length of about 10-30 nucleobases. In some embodiments, a second domain has a length of about 5-20 nucleobases. In some embodiments, a second domain has a length of about 7-16 nucleobases. In some embodiments, a second domain has a length of 7 nucleobases. In some embodiments, a second domain has a length of 8 nucleobases. In some embodiments, a second domain has a length of 9 nucleobases. In some embodiments, a second domain has a length of 10 nucleobases. In some embodiments, a second domain has a length of 11 nucleobases. In some embodiments, a second domain has a length of 12 nucleobases. In some embodiments, a second domain has a length of 13 nucleobases. In some embodiments, a second domain has a length of 14 nucleobases. In some embodiments, a second domain has a length of 15 nucleobases. In some embodiments, a second domain has a length of 16 nucleobases. In some embodiments, a second domain has a length of 17 nucleobases. In some embodiments, a second domain has a length of 18 nucleobases. In some embodiments, a second domain has a length of 19 nucleobases. In some embodiments, a second domain has a length of 20 nucleobases. In some embodiments, a second domain has a length of 21 or more nucleobases. In some embodiments, a second domain has the same length as a second portion.

In some embodiments, about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases in the second domain are each independently optionally substituted A, T, C, G, or U, or an optionally substituted tautomer of A, T, C, G or U. In some embodiments, each nucleobase in a second domain is independently optionally substituted A, T, C, G, or U, or an optionally substituted tautomer of A, T, C, G or U. In some embodiments, about 10-30 nucleobases in the second domain are each independently optionally substituted A, T, C, G, or U, or an optionally substituted tautomer of A, T, C, G or U. In some embodiments, about 10-25 nucleobases in the second domain are each independently optionally substituted A, T, C, G, or U, or an optionally substituted tautomer of A, T, C, G or U. In some embodiments, about 10 nucleobases in the second domain are each independently optionally substituted A, T, C, G, or U, or an optionally substituted tautomer of A, T, C, G or U. In some embodiments, about 11 nucleobases in the second domain are each independently optionally substituted A, T, C, G, or U, or an optionally substituted tautomer of A, T, C, G or U. In some embodiments, about 12 nucleobases in the second domain are each independently optionally substituted A, T, C, G, or U, or an optionally substituted tautomer of A, T, C, G or U. In some embodiments, about 13 nucleobases in the second domain are each independently optionally substituted A, T, C, G, or U, or an optionally substituted tautomer of A, T, C, G or U. In some embodiments, about 14 nucleobases in the second domain are each independently optionally substituted A, T, C, G, or U, or an optionally substituted tautomer of A, T, C, G or U. In some embodiments, about 15 nucleobases in the second domain are each independently optionally substituted A, T, C, G. or U, or an optionally substituted tautomer of A, T, C, G or U. In some embodiments, about 16 nucleobases in the second domain are each independently optionally substituted A, T, C, G, or U, or an optionally substituted tautomer of A, T, C, G or U. In some embodiments, about 17 nucleobases in the second domain are each independently optionally substituted A, T, C, G, or U, or an optionally substituted tautomer of A, T, C, G or U. In some embodiments, about 18 nucleobases in the second domain are each independently optionally substituted A, T, C, G, or U, or an optionally substituted tautomer of A, T, C, G or U. In some embodiments, about 19 nucleobases in the second domain are each independently optionally substituted A, T, C, G, or U, or an optionally substituted tautomer of A, T, C, G or U. In some embodiments, about 20 nucleobases in the second domain are each independently optionally substituted A, T, C, G, or U, or an optionally substituted tautomer of A, T, C, G or U. In some embodiments, about 21 or more nucleobases in the second domain are each independently optionally substituted A, T, C, G, or U, or an optionally substituted tautomer of A, T, C, G or U. In some embodiments, such nucleobases are each independently optionally substituted A, T, C, G or U. In some embodiments, such nucleobases are each independently A, T, C, G or U.

In some embodiments, a second domain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of an oligonucleotide. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

Base sequences of second domains are typically of sufficient complementarity to second portions so that second domains can form duplexes with second portions of target nucleic acids. In some embodiments, base sequence of a second domain is about 70%-100%, e.g., about or at least about 70%, 75%, 80%, 85%, 90%, 95%, or about 100%, complementary to the base sequence of the second portion. In some embodiments, the complementarity is about 70% or more. In some embodiments, the complementarity is about 75% or more. In some embodiments, the complementarity is about 80% or more. In some embodiments, the complementarity is about 85% or more. In some embodiments, the complementarity is about 90% or more. In some embodiments, the complementarity is about 95% or more. In some embodiments, the complementarity is about 100%. In some embodiments, the complementarity is about 100% except at the nucleoside opposite to target adenosine (No). In some embodiments, there are one or more mismatches. In some embodiments, there are one or more wobbles. In some embodiments, there are one or more bulges. In some embodiments, the second nucleobase from the 5′-end of the second domain is complementary to a nucleobase in the second portion. In some embodiments, the second nucleobase from the 3′-end of the second domain is complementary to a nucleobase in the second portion. In some embodiments, when an oligonucleotide is administered or delivered to a system comprising a target nucleic acid or a fragment thereof that comprises a second portion, a second domain can selectively form a duplex with a second portion. In some embodiments, when an oligonucleotide is administered or delivered to a system expressing a target nucleic acid or a fragment thereof that comprises a portion, a second domain can selectively form a duplex with a second portion. In some embodiments, base sequence of a second portion differentiates the second portion from the rest of a target nucleic acid. In some embodiments, base sequence of a second portion differentiates the second portion from the other transcripts in a system. In some embodiments, base sequence of a second portion differentiates a second portion from the other RNA in a system. In some embodiments, base sequence of a second portion differentiates the second portion from the other nucleic acids in a system.

As described herein, in some embodiments, a system is an in vitro system. In some embodiments, a system is an in vivo system. In some embodiments, a system is an ex vivo system. In some embodiments, a system is or comprises a cell. In some embodiments, a system is or comprises a population of cell. In some embodiments, a system is or comprises a tissue. In some embodiments, a system is or comprises an organ. In some embodiments, a system is or comprises a sample. In some embodiments, a system is or comprises an organism. In some embodiments, a system is an animal. In some embodiments, a system is a subject. In some embodiments, a system is a human.

In some embodiments, one or more (e.g., 1-20, 1, 2, 3, 4, 5, 6, 7, 8, 9, or 10, etc.) mismatches exist in a second domain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a second domain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a second domain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a second domain is fully complementary to a target nucleic acid.

In some embodiments, a second domain comprises one or more modified nucleobases.

In some embodiments, a second domain comprise a nucleoside opposite to a target adenosine, e.g., when the oligonucleotide forms a duplex with a target nucleic acid. In some embodiments, an opposite nucleobase is optionally substituted or protected U, or is an optionally substituted or protected tautomer of U. In some embodiments, an opposite nucleobase is U.

In some embodiments, an opposite nucleobase has weaker hydrogen bonding with a target adenine of a target adenosine compared to U. In some embodiments, an opposite nucleobase forms fewer hydrogen bonds with a target adenine of a target adenosine compared to U. In some embodiments, an opposite nucleobase forms one or more hydrogen bonds with one or more amino acid residues of a protein, e.g., ADAR, which residues form one or more hydrogen bonds with U opposite to a target adenosine. In some embodiments, an opposite nucleobase forms one or more hydrogen bonds with each amino acid residue of ADAR that forms one or more hydrogen bonds with U opposite to a target adenosine. In some embodiments, by weakening hydrogen boding with a target A and/or maintaining or enhancing interactions with proteins such as ADAR1, ADAR2, etc., certain opposite nucleobase facilitate and/or promote adenosine modification, e.g., by ADAR proteins such as ADAR1 and ADAR2.

In some embodiments, an opposite nucleobase is optionally substituted or protected C, or is an optionally substituted or protected tautomer of C. In some embodiments, an opposite nucleobase is C. In some embodiments, an opposite nucleobase is optionally substituted or protected A, or is an optionally substituted or protected tautomer of A. In some embodiments, an opposite nucleobase is A. In some embodiments, an opposite nucleobase is optionally substituted or protected nucleobase of pseudoisocytosine, or is an optionally substituted or protected tautomer of the nucleobase of pseudoisocytosine. In some embodiments, an opposite nucleobase is the nucleobase of pseudoisocytosine.

In some embodiments, a nucleoside, e.g., a nucleoside opposite to a target adenosine (may also be referred to as “an opposite nucleoside”) is abasic as described herein (e.g., having the structure of L010, L012, L028, etc.).

Many useful embodiments of modified nucleobases, e.g., for opposite nucleobases, are also described below. In some embodiments, as described herein (e.g., in various oligonucleotides), the present disclosure provides oligonucleotides comprising a nucleobase, e.g., of a nucleoside opposite to a target nucleoside such as A (N₀), N₁, N₋₁, etc., which is or comprises A, T, C, G, U, hypoxanthine, b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b003I, b004I, b014I, [3nT], [3ne5U], [3nfl5U], [3npry5U], [3ncn5U], [nathp6o8A], [ipr6o8A], [c7In], [c39z48In], [z2c3In], [z5C], zdnp or a nucleobase described in Table BA-1. In some embodiments, as described herein (e.g., in various oligonucleotides), the present disclosure provides oligonucleotides comprising a nucleobase, e.g., of a nucleoside opposite to a target nucleoside such as A, which is or comprises b001U, b002U, b003U, b004U, b005U, b006U, b007U, b008U, b009U, b011U, b012U, b013U, b001A, b002A, b003A, b001G, b002G, b001C, b002C, b003C, b004C, b005C, b006C, b007C, b008C, b009C, b002I, b003I, b004I, b014I, [3nT], [3ne5U], [3nfl5U], [3npry5U], [3ncn5U], [nathp6o8A], [ipr6o8A], [c7In], [c39z481n], [z2c3In], [z5C], and zdnp. In some embodiments, as described herein (e.g., in various oligonucleotides), the present disclosure provides oligonucleotides comprising a nucleobase, e.g., of a nucleoside opposite to a target nucleoside such as A, which is or comprises C, A, b007U, b001U, b001A, b002U, b001C, b003U, b002C, b004U, b003C, b005U, b0021, b006U, b0031, b008U, b009U, b002A, b003A, b001G, [3nT], [3ne5U], [3nfl5U], [3npry5U], [3ncn5U], [nathp6o8A], [ipr6o8A], [c7In], [c39z481n], [z2c3In], [z5C], or zdnp. In some embodiments, a nucleobase is C. In some embodiments, a nucleobase is A. In some embodiments, a nucleobase is hypoxanthine. In some embodiments, a nucleobase is b002I. In some embodiments, a nucleobase is b0031. In some embodiments, a nucleobase is b0041. In some embodiments, a nucleobase is b0141. In some embodiments, a nucleobase is b001C. In some embodiments, a nucleobase is b002C. In some embodiments, a nucleobase is b003C. In some embodiments, a nucleobase is b004C. In some embodiments, a nucleobase is b005C. In some embodiments, a nucleobase is b006C. In some embodiments, a nucleobase is b007C. In some embodiments, a nucleobase is b008C. In some embodiments, a nucleobase is b009C. In some embodiments, a nucleobase is b001U. In some embodiments, a nucleobase is b002U. In some embodiments, a nucleobase is b003U. In some embodiments, a nucleobase is b004U. In some embodiments, a nucleobase is b005U. In some embodiments, a nucleobase is b006U. In some embodiments, a nucleobase is b007U. In some embodiments, a nucleobase is b008U. In some embodiments, a nucleobase is b009U. In some embodiments, a nucleobase is b011U. In some embodiments, a nucleobase is b012U. In some embodiments, a nucleobase is b013U. In some embodiments, a nucleobase is b014U. In some embodiments, a nucleobase is b015U. In some embodiments, a nucleobase is b001A. In some embodiments, a nucleobase is b002A. In some embodiments, a nucleobase is b003A. In some embodiments, a nucleobase is b004A. In some embodiments, a nucleobase is b005A. In some embodiments, a nucleobase is b006A. In some embodiments, a nucleobase is b007A. In some embodiments, a nucleobase is b001G. In some embodiments, a nucleobase is b002G. In some embodiments, a nucleobase is [3nT]. In some embodiments, a nucleobase is [3ne5U]. In some embodiments, a nucleobase is [3nfl5U]. In some embodiments, a nucleobase is [3npry5U]. In some embodiments, a nucleobase is [3ncn5U]. In some embodiments, a nucleobase is [nathp6o8A]. In some embodiments, a nucleobase is [ipr6o8A]. In some embodiments, a nucleobase is [c7In]. In some embodiments, a nucleobase is [c39z481n]. In some embodiments, a nucleobase is [z2c3In]. In some embodiments, a nucleobase is [z5C]. In some embodiments, a nucleobase is or zdnp. In some embodiments, a nucleobase is selected from Table BA-1. In some embodiments, such a nucleobase is in N₀. In some embodiments, such a nucleobase is in N₁. In some embodiments, such a nucleobase is in N₋₁. In some embodiments, as those skilled in the art appreciate, a nucleobase is protected, e.g., for oligonucleotide synthesis. For example, in some embodiments, a nucleobase is protected b001A having the structure of

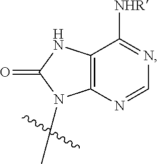

wherein R′ is as described herein. In some embodiments, R′ is —C(O)R. In some embodiments, R′ is —C(O)Ph.

In some embodiments, various modified nucleobases, e.g., b001A, b008U, etc., can provide improved adenosine editing efficiency when compared to a reference nucleobase (e.g., under comparable conditions including, e.g., in otherwise identical oligonucleotides, assessed in identical or comparable assays, etc.). In some embodiments, a reference nucleobase is U. In some embodiments, a reference nucleobase is T. In some embodiments, a reference nucleobase is C.

### Certain Modified Nucleobases

In some embodiments, BA is or comprises Ring BA or a tautomer thereof, wherein Ring BA is an optionally substituted, 5-20 membered, monocyclic, bicyclic or polycyclic ring having 0-10 heteroatoms. In some embodiments, Ring BA is or comprises an optionally substituted, 5-20 membered, monocyclic, bicyclic or polycyclic having 1-10 heteroatoms, wherein at least one heteroatom is nitrogen. In some embodiments, Ring BA is saturated. In some embodiments, Ring BA comprises one or more unsaturation. In some embodiments, Ring BA is partially unsaturated. In some embodiments, Ring BA is aromatic.

In some embodiments, BA is or comprises Ring BA, wherein Ring BA is an optionally substituted, 5-20 membered, monocyclic, bicyclic or polycyclic ring having 0-10 heteroatoms. In some embodiments, Ring BA is or comprises an optionally substituted, 5-20 membered, monocyclic, bicyclic or polycyclic having 1-10 heteroatoms, wherein at least one heteroatom is nitrogen. In some embodiments, Ring BA is saturated. In some embodiments, Ring BA comprises one or more unsaturation. In some embodiments, Ring BA is partially unsaturated. In some embodiments, Ring BA is aromatic.

In some embodiments, BA is or comprises Ring BA. In some embodiments, BA is Ring BA. In some embodiments, BA is or comprises a tautomer of Ring BA. In some embodiments, BA is a tautomer of Ring BA.

In some embodiments, structures of the present disclosure contain one or more optionally substituted rings (e.g., Ring BA, —Cy—, Ring BA^{A}, R, formed by R groups taken together, etc.). In some embodiments, a ring is an optionally substituted C₃₋₃₀, C₃₋₂₀, C₃₋₁₅, C₃₋₁₀, C₃₋₉, C₃₋₈, C₃₋₇, C₃₋₆, C₅₋₅₀, C₅₋₂₀, C₅₋₁₅, C₅₋₁₀, C₅₋₉, C₅₋₈, C₅₋₇, C₅₋₆, or 3-30 (e.g., 3-30, 3-20, 3-15, 3-10, 3-9, 3-8, 3-7, 3-6, 5-50, 5-20, 5-15, 5- 10, 5-9, 5-8, 5-7, 5-6, or 1, 2, 3, 4, 5, 6, 7, 8, 9, 10, 11, 12, 13, 14, 15, 16, 17, 18, 19, 20, 25, 30, etc.) membered monocyclic, bicyclic or polycyclic ring having 0-10 (e.g., 1-10, 1-5, 0, 1, 2, 3, 4, 5, 6, 7, 8, 9, 10, etc.) heteroatoms. In some embodiments, a ring is an optionally substituted 3-10 membered monocyclic or bicyclic, saturated, partially saturated or aromatic ring having 0-3 heteroatoms. In some embodiments, a ring is substituted. In some embodiments, a ring is not substituted. In some embodiments, a ring is 3, 4, 5, 6, 7, 8. 9, or 10 membered. In some embodiments, a ring is 5, 6, or 7-membered. In some embodiments, a ring is 5-membered. In some embodiments, a ring is 6-membered. In some embodiments, a ring is 7-membered. In some embodiments, a ring is monocyclic. In some embodiments, a ring is bicyclic. In some embodiments, a ring is polycyclic. In some embodiments, a ring is saturated. In some embodiments, a ring contains at least one unsaturation. In some embodiments, a ring is partially unsaturated. In some embodiments, a ring is aromatic. In some embodiments, a ring has 0-5 heteroatoms. In some embodiments, a ring has 1-5 heteroatoms. In some embodiments, a ring has one or more heteroatoms. In some embodiments, a ring has 1 heteroatom. In some embodiments, a ring has 2 heteroatoms. In some embodiments, a ring has 3 heteroatoms. In some embodiments, a ring has 4 heteroatoms. In some embodiments, a ring has 5 heteroatoms. In some embodiments, a heteroatom is nitrogen. In some embodiments, a heteroatom is oxygen. In some embodiments, a ring is substituted, e.g., substituted with one or more alkyl groups and optionally one or more other substituents as described herein. In some embodiments, a substituent is methyl.

In some embodiments, each monocyclic ring unit of a monocyclic, bicyclic, or polycyclic ring of the present disclosure (e.g., Ring BA, —Cy—, Ring BA^{A}, R, formed by R groups taken together, etc.) is independently an optionally substituted 5-7 membered, saturated, partially unsaturated or aromatic ring having 0-5 heteroatoms. In some embodiments, one or more monocyclic units independently comprise one or more unsaturation. In some embodiments, one or more monocyclic units are saturated. In some embodiments, one or more monocyclic units are partially saturated. In some embodiments, one or more monocyclic units are aromatic. In some embodiments, one or more monocyclic units independently have 1-5 heteroatoms. In some embodiments, one or more monocyclic units independently have at least one nitrogen atom. In some embodiments, each monocyclic unit is independently 5- or 6-membered. In some embodiments, a monocyclic unit is 5-membered. In some embodiments, a monocyclic unit is 5-membered and has 1-2 nitrogen atom. In some embodiments, a monocyclic unit is 6-membered. In some embodiments, a monocyclic unit is 6-membered and has 1-2 nitrogen atom. Rings and monocyclic units thereof are optionally substituted unless otherwise specified.

Without the intention to be limited by any particular theory, the present disclosure recognizes that in some embodiment, structures of nucleobases (e.g. BA) can impact interactions with proteins (e.g., ADAR proteins such as ADAR1, ADAR2, etc.). In some embodiments, provided oligonucleotides comprise nucleobases that can facility interaction of an oligonucleotide with an enzyme, e.g., ADAR1. In some embodiments, provided oligonucleotides comprise nucleobases that may reduce strength of base pairing (e.g., compared to A-T/U or C-G). In some embodiments, the present disclosure recognizes that by maintaining and/or enhancing interactions (e.g., hydrogen bonding) of a first nucleobase with a protein (e.g., an enzyme like ADAR1) and/or reducing interactions (e.g., hydrogen bonding) of a first nucleobase with its corresponding nucleobase (e.g., A) on the other strand in a duplex, modification of the corresponding nucleobase by a protein (e.g., an enzyme like ADAR1) can be significantly improved. In some embodiments, the present disclosure provides oligonucleotides comprises such a first nucleobase (e.g., various embodiments of BA described herein). Exemplary embodiments of such as a first nucleobase are as described herein. In some embodiments, when an oligonucleotide comprising such a first nucleobase is aligned with another nucleic acid for maximum complementarity, the first nucleobase is opposite to A. In some embodiments, such an A opposite to the first nucleobase, as exemplified in many embodiments of the present disclosure, can be efficiently modified using technologies of the present disclosure.

In some embodiments, Ring BA comprises a moiety [symbol] X² [symbol] X³ [symbol] , wherein each variable is independently as described herein. In some embodiments, Ring BA comprises a moiety [symbol] X² [symbol] X³ [symbol] X⁴ [symbol] , wherein each variable is independently as described herein. In some embodiments, Ring BA comprises a moiety —X₁( [symbol] ) [symbol] X² [symbol] X³ [symbol] , wherein each variable is independently as described herein. In some embodiments, Ring BA comprises a moiety —X¹( [symbol] ) [symbol] X² [symbol] X³ [symbol] X⁴ [symbol] , wherein each variable is independently as described herein. In some embodiments, X¹ is bonded to a sugar. In some embodiments, X¹is —N(−)—. In some embodiments, X¹ is —C(═)—. In some embodiments, X² is —C(O)—. In some embodiments, X³ is —NH—. In some embodiments, X⁴ is not —C(O)—. In some embodiments, X⁴ is —C(O)—, and forms an intramolecular hydrogen bond, e.g., with a moiety of the same nucleotidic unit (e.g., within the same BA unit (e.g., with a hydrogen bond donor (e.g., —OH, SH, etc.) of X). In some embodiments, X⁴ is —C(═NH)—. In some embodiments, Ring BA comprises a moiety [symbol] X⁴′ [symbol] X^{5′} [symbol] , wherein each variable is independently as described herein. In some embodiments, X⁴′ is —C(O)—. In some embodiments, X⁵′ is —NH—.

In some embodiments, BA is optionally substituted or protected C or a tautomer thereof. In some embodiments, BA is optionally substituted or optionally protected C. In some embodiments, BA is an optionally substituted or optionally protected tautomer of C. In some embodiments, BA is C. In some embodiments, BA is substituted C. In some embodiments, BA is protected C. In some embodiments, BA is an substituted tautomer of C. In some embodiments, BA is an protected tautomer of C.

In some embodiments, Ring BA has the structure of formula BA-I:

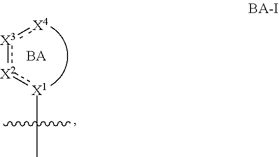

wherein:
Ring BA is an optionally substituted, 5-20 membered, monocyclic, bicyclic or polycyclic, saturated, partially saturated or aromatic ring having 1-10 heteroatoms;
each [symbol] is independent a single or double bond;
X¹ is —N(−)— or —C(−)═;
X² is —C(W^{X2})—, —C(R^{B2})═, —C(OR^{B2})═, —N═ or optionally substituted —CH═, wherein R^{B2} is halogen, —CN, —NO₂, or -L^{B2}-R′, and W^{X2} is O, S or Se;
X³ is —C(R^{B3})═, —N(R^{B3})—, —N═ or optionally substituted —NH— or —CH═, wherein R^{B3} is halogen, —CN, —NO₂, or -L^{B3}-R′;
X⁴ is —C(R^{B4})═, —C(—N(R^{B4})₂)═, —C(R^{B4})₂—, —C(W^{X4})—, —C(═NR^{B4})— or optionally substituted —CH═ or —CH₂—, wherein each R^{B4} is independently halogen, —CN, —NO₂, or -L^{B4}-R^{B41}, or two R^{B4} on the same atom are taken together to form ═O, ═C(-L^{B4}-R^{B41})₂, ═N-L^{B4}-R^{B41}, or optionally substituted ═CH₂ or ═NH, wherein each R^{B41} is independently R′, and W^{X4} is O, S or Se;
each of L^{B2}, L^{B3}, and L^{B4} is independently L^{B};
each L^{B} is independently a covalent bond, or an optionally substituted bivalent C₁₋₁₀ saturated or partially unsaturated chain having 0-6 heteroatoms, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted, 3-20 membered, monocyclic, bicyclic or polycyclic ring having 0-10 heteroatoms;
each R′ is independently —R, —C(O)R, —C(O)OR, —C(O)N(R)₂, or —SO₂R; and
each R is independently —H, or an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, Ring BA (e.g., one of formula BA-I) has the structure of formula BA-I-a:

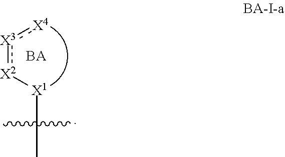

In some embodiments, Ring BA (e.g., one of formula BA-I, BA-I-a, etc.)has the structure of formula BA-I-b:

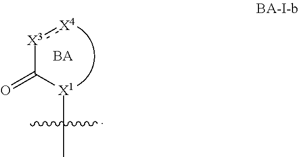

In some embodiments, Ring BA (e.g., one of formula BA-I, BA-I-a, etc.)has the structure of formula BA-I-c:

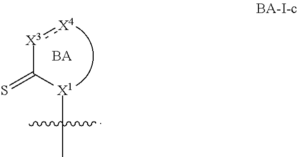

In some embodiments, Ring BA (e.g., one of formula BA-I, BA-I-a, etc.)has the structure of formula BA-I-d:

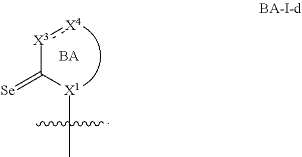

In some embodiments, Ring BA (e.g., one of formula BA-I) has the structure of formula BA-IL:

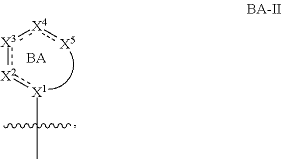

wherein:
X⁵ is —C(R^{B5})₂—, —N(R^{B5})—, —C(R^{B5})═, —C(W^{X5})—, —N═ or optionally substituted —CH₂—, —NH— or —CH═, wherein each R^{B5} is independently halogen, —CN, —NO₂, or -L^{B5}-R^{B51}, wherein RB is —R′, —N(R′)₂, —OR′, or —SR′, and W^{X5} is O, S, or Se;
L^{B5} is L^{B}; and
each other variable is independently as described herein.

In some embodiments, Ring BA (e.g., one of formula BA-I, BA-I-a, BA-II, etc.) has the structure of formula BA-II-a:

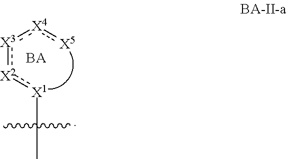

In some embodiments, Ring BA (e.g., one of formula BA-I, BA-I-a, BA-I-b, BA-II, BA-II-a, etc.) has the structure of formula BA-II-b:

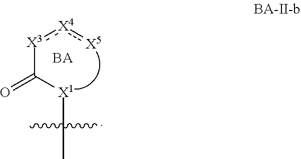

In some embodiments, Ring BA (e.g., one of formula BA-I. BA-I-a, BA-I-b, BA-II, BA-II-a, etc.) has the structure of formula BA-II-b:

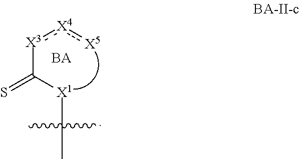

In some embodiments, Ring BA (e.g., one of formula BA-I. BA-I-a, BA-I-b, BA-II, BA-II-a, etc.) has the structure of formula BA-II-b:

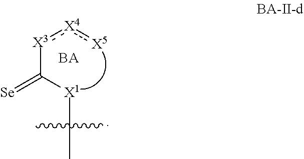

In some embodiments, Ring BA (e.g., one of formula BA-I, BA-II, etc.) has the structure of formula BA-Ill:

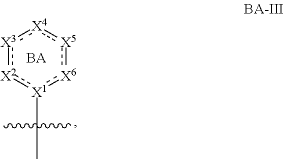

wherein:
X⁶ is —C(R^{B6})═, —C(OR^{B6})═, —C(R^{B6})₂—, —C(W^{X6})—, —N═ or optionally substituted —CH═ or —CH₂—, wherein each R^{B6} is independently halogen, —CN, —NO₂, or -L^{B6}-R^{B61}, or two R^{B6} on the same atom are taken together to form ═O, ═C(-L^{B6}-R^{B61})₂, ═N-L^{B6}-R^{B61}, or optionally substituted ═CH₂ or ═NH, wherein each R^{B61} is independently R′, and W^{X6} is O, S, or Se;
L^{B6} is L^{B}; and
each other variable is independently as described herein.

In some embodiments, Ring BA (e.g., one of formula BA-I, BA-I-a, BA-II, BA-II-a, BA-III, etc.) has the structure of formula BA-III-a:

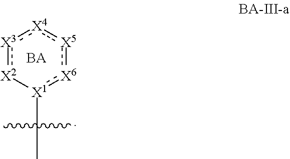

In some embodiments, Ring BA (e.g., one of formula BA-I, BA-I-a, BA-I-b, BA-II, BA-II-a, BA-II-b, BA-III, BA-III-a, etc.) has the structure of formula BA-III-b:

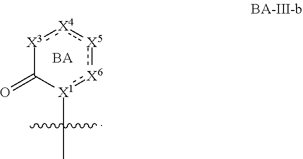

In some embodiments, Ring BA (e.g., one of formula BA-I, BA-I-a, BA-I-b, BA-II, BA-II-a, BA-II-b, BA-III, BA-III-a, etc.) has the structure of formula BA-III-c:

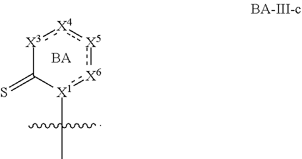

In some embodiments, Ring BA (e.g., one of formula BA-I, BA-I-a, BA-I-b, BA-II, BA-II-a, BA-II-b, BA-III, BA-III-a, etc.) has the structure of formula BA-III-d:

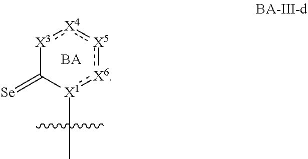

In some embodiments, Ring BA (e.g., one of formula BA-I, BA-I-a, BA-I-b, BA-II, BA-II-a, BA-II-b, BA-III, BA-III-a, etc.) has the structure of formula BA-III-e:

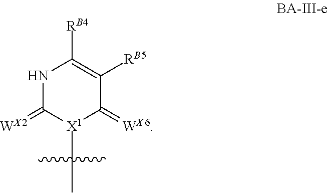

In some embodiments, X¹ is N, and each of R^{B4} and R^{B5} is independently halogen or optionally substituted C₁₋₁₀ alkyl. In some embodiments, each of R^{B4} and R^{B5} is independently halogen or optionally substituted C₁₋₆ alkyl. In some embodiments, each of R^{B4} and R^{B5} is independently halogen or C₁₋₆ alkyl. In some embodiments, each of R^{B4} and R^{B5} is independently halogen or C₁₋₄ alkyl. In some embodiments, R^{B4} is —H. In some embodiments, R^{B4} is not —H. In some embodiments, R^{B4} is halogen. In some embodiments, R^{B4} is —F. In some embodiments, R^{B4} is —Cl. In some embodiments, R^{B4} is —Br. In some embodiments, R^{B4} is —CN. In some embodiments, R^{B4} is —NO₂. In some embodiments, R^{B4} is optionally substituted C₁₋₆(e.g., C_{1_5}, C₁₋₄, C₁₋₃, C₁₋₂, C₁, C₂, C₃, C₄, C₅, C₆, etc.) aliphatic. In some embodiments, R^{B4} is optionally substituted C₁₋₄ aliphatic. In some embodiments, R^{B4} is optionally substituted C₁₋₃ aliphatic. In some embodiments, R^{B4} is optionally substituted C₁₋₂ aliphatic. In some embodiments, R^{B4} is C₁₋₆ (e.g., C₁₋₅, C₁₋₄, C₁₋₃, C₁₋₂, C₁, C₂, C₃, C₄, C₅, C₆, etc.) aliphatic optionally substituted halogen. In some embodiments, R^{B4} is C₁₋₄ aliphatic optionally substituted halogen. In some embodiments, R^{B4} IS C₁₋₃ aliphatic optionally substituted halogen. In some embodiments, R^{B4} is C₁₋₂ aliphatic optionally substituted halogen. In some embodiments, R^{B4} is C₁₋₆(e.g., C₁₋₅, C₁₋₄, C₁₋₃, C₁₋₂, C₁, C₂, C₃, C₄, C₅, C₆, etc.) aliphatic. In some embodiments, R^{B4} is C₁₋₄ aliphatic. In some embodiments, R^{B4}is C₁₋₃ aliphatic. In some embodiments, R^{B4} is C₁₋₂ aliphatic. In some embodiments, R^{B4} is optionally substituted C₁₋₆ (e.g., C₁₋₅, C₁₋₄, C₁₋₃, C₁₋₂, C₁, C₂, C₃, C₄, C₅, C₆, etc.) alkyl. In some embodiments, R^{B4} is optionally substituted C₁₋₄ alkyl. In some embodiments, R^{B4} is optionally substituted C₁₋₃ alkyl. In some embodiments, R^{B4} is optionally substituted C₁₋₂ alkyl. In some embodiments, R^{B4} is C₁₋₆(e.g., C₁₋₅, C₁₋₄, C₁₋₃, C₁₋₂, C₁, C₂, C₃, C₄, C₅, C₆, etc.) alkyl optionally substituted with halogen. In some embodiments, R^{B4} is C₁₋₄ alkyl optionally substituted with halogen. In some embodiments, R^{B4} is C₁₋₃ alkyl optionally substituted with halogen. In some embodiments, R^{B4} is C₁₋₂ alkyl optionally substituted with halogen. In some embodiments, R^{B4} is C₁₋₆ (e.g., C₁₋₅, C₁₋₄, C₁₋₃, C₁₋₂, C₁, C₂, C₃, C₄, C₅, C₆, etc.) alkyl. In some embodiments, R^{B4} is C₁₋₄ alkyl. In some embodiments, R^{B4} IS C₁₋₄ alkyl. In some embodiments, R^{B4} IS C₁₋₃ alkyl. In some embodiments, R^{B4} IS C₁₋₂ alkyl. In some embodiments, R^{B4} is methyl. In some embodiments, R^{B4} is ethyl. In some embodiments, R^{B5} is —H. In some embodiments, R^{B5} is not —H. In some embodiments, R^{B5} is halogen. In some embodiments, R^{B5} is —F. In some embodiments, R^{B5} is —Cl . In some embodiments, R^{B5} is —Br. In some embodiments, R^{B5} is —CN. In some embodiments, R^{B5} is —NO₂. In some embodiments, R^{B5} is optionally substituted C₁₋₆(e.g., C₁₋₅, C₁₋₄, C₁₋₃, C₁₋₂, C₁, C₂, C₃, C₄, C₅, C₆, etc.) aliphatic. In some embodiments, R^{B5} is optionally substituted C₁₋₄ aliphatic. In some embodiments, R^{B5} is optionally substituted C₁₋₃ aliphatic. In some embodiments, R^{B5} is optionally substituted C₁₋₂ aliphatic. In some embodiments, R^{B5} IS C₁₋₆(e.g., C₁₋₅, C₁₋₄, C₁₋₃, C₁₋₂, C₁, C₂, C₃, C₄, C₅, C₆, etc.) aliphatic optionally substituted halogen. In some embodiments, R^{B5} is C₁₋₄ aliphatic optionally substituted halogen. In some embodiments, R^{B5} is C₁₋₃ aliphatic optionally substituted halogen. In some embodiments, R^{B5} IS C₁₋₂ aliphatic optionally substituted halogen. In some embodiments, R^{B5} is C₁₋₆(e.g., C₁₋₅, C₁₋₄, C₁₋₃, C₁₋₂, C₁, C₂, C₃, C₄, C₅, C₆, etc.) aliphatic. In some embodiments, R^{B5} is C₁₋₄ aliphatic. In some embodiments, R^{B5} IS C₁₋₃ aliphatic. In some embodiments, R^{B5} is C₁₋₂ aliphatic. In some embodiments, R^{B5} is optionally substituted C₁₋₆ (e.g., C₁₋₅, C₁₋₄, C₁₋₃, C₁₋₂, C₁, C₂, C₃, C₄, C₅, C₆, etc.) alkyl. In some embodiments, R^{B5} is optionally substituted C₁₋₄ alkyl. In some embodiments, R^{B5} is optionally substituted C₁₋₃ alkyl. In some embodiments, R^{B5} is optionally substituted C₁₋₂ alkyl. In some embodiments, R^{B5} is C₁₋₆ (e.g., C₁₋₅, C₁₋₄, C₁₋₃, C₁₋₂, C₁, C₂, C₃, C₄, C₅, C₆, etc.) alkyl optionally substituted with halogen. In some embodiments, R^{B5} is C₁₋₄ alkyl optionally substituted with halogen. In some embodiments, R^{B5} is C₁₋₃ alkyl optionally substituted with halogen. In some embodiments, R^{B5} IS C₁₋₂ alkyl optionally substituted with halogen. In some embodiments, R^{B5} is C₁₋₆ (e.g., C₁₋₅, C₁₋₄, C₁₋₃, C₁₋₂, C₁, C₂, C₃, C₄, C₅, C₆, etc.) alkyl. In some embodiments, R^{B5} is C₁₋₄ alkyl. In some embodiments, R^{B5} is C₁₋₄ alkyl. In some embodiments, R^{B5} is C₁₋₃ alkyl. In some embodiments, R^{B5} is C₁₋₂ alkyl. In some embodiments, R^{B5} is methyl. In some embodiments, R^{B5} is ethyl. In some embodiments, R^{B5} is —C≡CH. In some embodiments, R^{B5} is —C≡CHCH₃. In some embodiments, W^{X2} and W^{X6} are each O. In some embodiments, Ring BA is optionally substituted

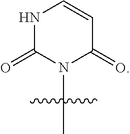 [SMILES: *n1c(=O)cc[nH]c1=O]

In some embodiments, Ring BA is substituted

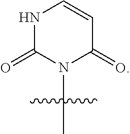 [SMILES: *n1c(=O)cc[nH]c1=O]

In some embodiments, Ring BA is

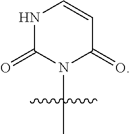 [SMILES: *n1c(=O)cc[nH]c1=O]

In some embodiments, Ring BA is

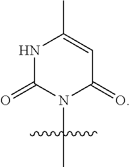 [SMILES: *n1c(=O)cc(C)[nH]c1=O]

In some embodiments, Ring BA is

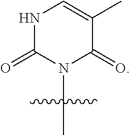 [SMILES: *n1c(=O)[nH]cc(C)c1=O]

In some embodiments, Ring BA is

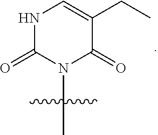 [SMILES: *n1c(=O)[nH]cc(CC)c1=O]

In some embodiments, Ring BA is

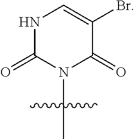 [SMILES: *n1c(=O)[nH]cc(Br)c1=O]

In some embodiments, Ring BA is

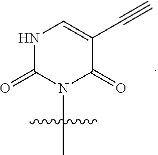 [SMILES: *n1c(=O)[nH]cc(C#C)c1=O]

In some embodiments, Ring BA is

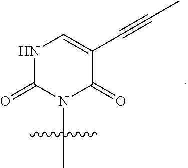 [SMILES: *n1c(=O)[nH]cc(C#CC)c1=O]

In some embodiments, Ring BA is

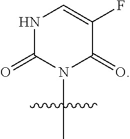 [SMILES: *n1c(=O)[nH]cc(F)c1=O]

In some embodiments, Ring BA is

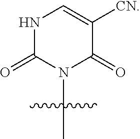 [SMILES: *n1c(=O)[nH]cc(C#N)c1=O]

In some embodiments, Ring BA is

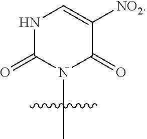 [SMILES: *n1c(=O)[nH]cc([N+](=O)[O-])c1=O]

In some embodiments, Ring BA (e.g., one of formula BA-I, BA-II, etc.) has the structure of formula BA-IV:

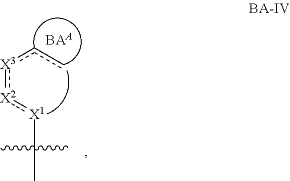

wherein:
Ring BA^{A} is an optionally substituted 5-14 membered, monocyclic, bicyclic or polycyclic ring having 0-5 heteroatoms, and
each other variable is independently as described herein.

In some embodiments, Ring BA (e.g., one of formula BA-I, BA-I-a, BA-II, BA-II-a, etc.) has the structure of formula BA-IV-a:

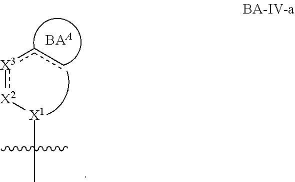

In some embodiments, Ring BA (e.g., one of formula BA-I, BA-I-a, BA-II, BA-II-a, etc.) has the structure of formula BA-IV-b:

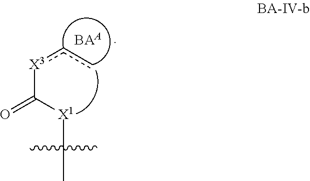

In some embodiments, Ring BA (e.g., one of formula BA-I, BA-Il, BA-Ill, BA-IV, etc.) has the structure of formula BA-V:

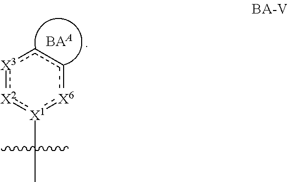

In some embodiments, Ring BA (e.g., one of formula BA-I, BA-I-a, BA-Il, BA-II-a, BA-III, BA-III-a, BA-IV, BA-IV-a, BA-V, etc.) has the structure of formula BA-V-a:

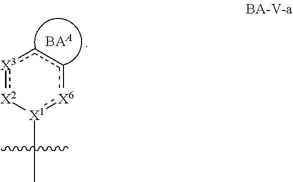

In some embodiments, Ring BA (e.g., one of formula BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, etc.) has the structure of formula BA-V-a:

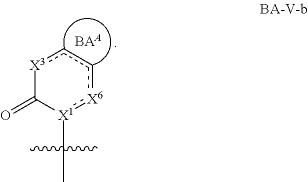

In some embodiments, Ring BA has the structure of formula BA-VI:

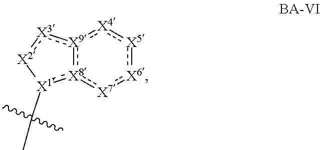

wherein:
each [symbol] is independent a single or double bond;
X¹′ is —N(−)— or —C(−)═;
X²′ is —C(W^{X2}′)—, —C(R^{B2}′)═, —C(OR^{B2′})═, —N═, or optionally substituted —CH═ or —CH₂—, wherein R^{B2}′ is halogen, —CN, —NO₂, or -L^{B2}′-R′, and W^{X2}′ is O, S or Se;
X^{3′} is —N(R^{B3}′)═, —N═, —C(R^{B3}′)═ or optionally substituted —NH— or —CH═, wherein R^{B3}′ is halogen, —CN, —NO₂, or -L^{B3}′-R′;
X⁴′ is —C(R^{B4}′)═, —C(OR^{B4}′)═, —C(N(R^{B4}′)₂)═, —C(R^{B4}′)₂, —C(W^{X4}′)—, —C(═NR^{B4}′)—, —N(R^{B4}′)—, —N═, or optionally substituted —CH═, —NH— or —CH₂—, wherein each R^{B4}′ is independently halogen, —CN, —NO₂, or -L^{B4′}-R^{B41}′, or two R^{B4}′ on the same atom are taken together to form ═O, ═C(-L^{B4}′-R^{B41}′)₂, ═N-L^{B4}′-R^{B41}′, or optionally substituted ═CH₂ or ═NH, wherein each R^{B41}′ is independently —R′, and W^{X4}′ is O, S or Se;
X⁵′ is —C(R^{B5}′)₂—, —N(R^{B5}′)—, —C(R^{B5}′)═, —C(W^{X5}′)—, —N═, or optionally substituted —NH—, —CH₂—, or —CH═, wherein each R^{B5}′ is independently halogen, —CN, —NO₂, or -L^{B5}′-R^{B51}, wherein R^{B51}′ is —R′, —N(R′)₂, —OR′, or —SR′, and W^{X5}′ is O, S, or Se;
X^{6′} is ——C(R^{B6}′)═, —C(OR^{B6})═, —C(R^{B6})₂—, —C(W^{X6}′)—, —C(—N(R^{B6}′)₂)═, —N═ or optionally substituted —NH—, —CH₂— or —CH═, wherein each R^{B6}′ is independently halogen, —CN, —NO₂, or -L^{B6}′-R^{B61}′ or two R^{B6}′ on the same atom are taken together to form ═O, ═C(-L^{B6}′-R^{B61}′)₂—, ═N-L^{B6}′-R^{B61}′, or optionally substituted ═CH₂ or ═NH, wherein each R^{B61}′ is independently R′, and W^{X6}′ is O, S or Se;
X⁷′ is —C(R^{B7}′)═, —C(OR^{B7}′)═, —C(R^{B7}′)₂—, —C(W^{X7}′)—, —C(—N(R^{B7}′)₂)═, —N(R^{B7}′), —N═ or optionally substituted —NH—, —CH₂— or —CH═, wherein each R^{B7}′ is independently halogen, —CN, —NO₂, or -L^{B7}′-R^{B71}′, or two R^{B7}′ on the same atom are taken together to form ═O, ═C(-L^{B7}′-R^{B71}′)₂, ═N-L^{B7}′- R^{B71}′, or optionally substituted ═CH₂ or ═NH, wherein each R^{B71}′ is independently R′, and wherein W^{X7}′ is O, S, or Se;
each of X⁸′ and X⁹′ is independently C or N;
each of L^{B2}′, L^{B3}′, L^{B4}′, L^{B5}′, L^{B6}′ and L^{B7}′ is independently L^{B}; and
each other variable is independently as described herein.

In some embodiments, [symbol] is a single bond. In some embodiments, [symbol] is a double bond.

In some embodiments, X¹ is —(N−)—. In some embodiments, X¹ is —C(−)═.

In some embodiments, W^{X2} is O. In some embodiments, W^{X2} is S. In some embodiments, W^{X2} is Se. In some embodiments, X² is —C(O)—. In some embodiments, X² is —C(S)—. In some embodiments, X² is —C(Se)—. In some embodiments, X² is —C(R^{B2})═. In some embodiments, X² is —C(OR^{B2})═. In some embodiments, X² is —C(OR^{B2})═, wherein R^{B2} is -L^{B2}-R′. In some embodiments, X² is optionally substituted —CH═. In some embodiments, X² is —CH═. In some embodiments, X² is —C(R^{B2})═, wherein R^{B2} is halogen, —CN, —NO₂, or -L^{B2}-R′. In some embodiments, X² is —C(OR^{B2})═, wherein R^{B2} is -L^{B2}-R′. In some embodiments, X² is —N═.

In some embodiments, L^{B2} is a covalent bond.

In some embodiments, R^{B2} is a protecting group, e.g., a hydroxyl protecting group suitable for oligonucleotide synthesis. In some embodiments, R^{B2} is R′. In some embodiments. R^{B2} is —H. In some embodiments, R^{B2} is halogen. In some embodiments, R^{B2} is —F. In some embodiments, R^{B2} is —Cl . In some embodiments, R^{B2} is —Br. In some embodiments, R^{B2} is —I. In some embodiments, R^{B2} is —CN. In some embodiments, R^{B2} is —NO₂. In some embodiments, R^{B2} is -L^{B2}-R′.

In some embodiments, X³ is —C(R^{B2})═. In some embodiments, X³ is optionally substituted —CH═. In some embodiments, X³ is —CH═. In some embodiments, X³ is —N(R^{B3})—. In some embodiments, X³ is —N(R^{B3})—, wherein R^{B3} is -L^{B3}-R′. In some embodiments, X³ is —N(R^{B3})— wherein R^{B3} is -L^{B3}-R′. In some embodiments, X³ is optionally substituted —NH—. In some embodiments, X³ is —NH—. In some embodiments, X³ is —N═.

In some embodiments, L^{B3} is a covalent bond.

In some embodiments, R^{B3} is a protecting group, e.g., an amino protecting group suitable for oligonucleotide synthesis (e.g., Bz). In some embodiments, R^{B3} is R′. In some embodiments, R^{B3} is —C(O)R. In some embodiments, R^{B3} is R. In some embodiments, R^{B3} is —H. In some embodiments, R^{B3} is halogen. In some embodiments, R^{B3} is —F. In some embodiments. R^{B3} is —Cl. In some embodiments, R^{B3} is —Br. In some embodiments, R^{B3} is —I. In some embodiments, R^{B3} is —CN. In some embodiments, R^{B3} is —NO₂. In some embodiments, R^{B3} is -L^{B3}-R′.

In some embodiments, X⁴ is —C(R^{B4})═. In some embodiments, X⁴ is —C(R)═. In some embodiments, X⁴ is —CH═. In some embodiments, X⁴ is —CH═. In some embodiments, X⁴ is —C(OR^{B4})═. In some embodiments, X⁴ is —C(—N(R^{B4})₂)═. In some embodiments, X⁴ is —C(—N(R^{B4})₂)═, wherein each R^{B4} is independently -L^{B4}-R^{B41}. In some embodiments, X⁴ is —C(—NHR^{B4})═. In some embodiments, X⁴ is —C(—NHR′)═. In some embodiments, X⁴ is —C(—NHR′)═. In some embodiments, X⁴ is optionally substituted —C(—NH₂)═. In some embodiments, X⁴ is —C(—NH₂)═. In some embodiments, X⁴ is —C(—NHC(O)R)═. In some embodiments, X⁴ is —C(R^{B4})₂—. In some embodiments, X⁴ is optionally substituted —CH₂—. In some embodiments, X⁴ is —CH—. In some embodiments, X⁴ is —C(O)—. In some embodiments, X⁴ is —C(S)—. In some embodiments, X⁴ is —C(Se)—. In some embodiments, X⁴ is —C(O)—, wherein O forms a intramolecular hydrogen bond. In some embodiments, O forms a hydrogen bond with a hydrogen bond donor of X⁵ of the same BA. In some embodiments, X⁴ is —C(═NR^{B4})—. In some embodiments, X⁴ is —C((═NR^{B4})—, wherein N forms a intramolecular hydrogen bond. In some embodiments, N forms a hydrogen bond with a hydrogen bond donor of X⁵ of the same BA.

In some embodiments, R^{B4} is -L^{B4}-R^{B41}. In some embodiments, two R^{B4} on the same atom are taken together to form ═O, ═C(-L^{B4}-R^{B41})₂, ═N-L^{B4}- R^{B41}, or optionally substituted ═CH₂ or ═NH.

In some embodiments, two R^{B4} on the same atom are taken together to form ═O. In some embodiments, two R^{B4} on the same atom are taken together to form ═C(-L^{B4}-R^{B41})₂. In some embodiments, ═C(-L^{B4}-R^{B41})₂ is ═CH-L^{B4}-R^{B41}. In some embodiments, ═C(_L^{B4}-R^{B41})₂ is ═CHR′. In some embodiments, ═C(-L^{B4}-R^{B41})₂ is ═CHR. In some embodiments, two R^{B4} on the same atom are taken together to form ═N-L^{B4}-R^{B41}. In some embodiments, =N-L^{B4}-R^{B41} is ═N—R. In some embodiments, two R^{B4} on the same atom are taken together to form ═CH₂. In some embodiments, two R^{B4} on the same atom are taken together to form ═NH. In some embodiments, a formed group is a suitable protecting group, e.g., amino protecting group, for oligonucleotide synthesis.

In some embodiments, X⁴ is —C(—N═C(-L^{B4}-R^{B41})₂)═. In some embodiments, X⁴ is —C(—N═CH-L^{B4}-R^{B41})═. In some embodiments, X⁴ is —C(—N═CH—N(CH₃)₂)═.

In some embodiments, R of X⁴ (e.g., of —C(═N—R)—, ═C(R)—, etc.) are optionally taken together with another R, e.g., of X⁵, to form a ring as described herein.

In some embodiments, R^{B4} is R′. In some embodiments, R^{B4} is R. In some embodiments, R^{B4} is —H. In some embodiments, R^{B4} is halogen. In some embodiments, R^{B4} is —F. In some embodiments, R^{B4} is —Cl. In some embodiments, R^{B4} is —Br. In some embodiments, R^{B4} is —I. In some embodiments, R^{B4} is —CN. In some embodiments, R^{B4} is —NO₂. In some embodiments, R^{B4} is -L^{B4}-R′. In some embodiments, R^{B4} is a protecting group, e.g., an amino or hydroxyl protecting group suitable for oligonucleotide synthesis. In some embodiments, R^{B4} is R′. In some embodiments, R^{B4} is —CH₂CH₂-(4-nitrophenyl).

In some embodiments, L^{B4} is a covalent bond. In some embodiments, L^{B4} is not a covalent bond. In some embodiments, at least one methylene unit is replaced with —C(O)—. In some embodiments, at least one methylene unit is replaced with —C(O)N(R′)—. In some embodiments, at least one methylene unit is replaced with —N(R′)—. In some embodiments, at least one methylene unit is replaced with —NH—. In some embodiments, L^{B4} is or comprises optionally substituted —N═CH—.

In some embodiments, R^{B41} is R′. In some embodiments, R^{B41} is —H. In some embodiments, R^{B41} is R. In some embodiments. R is optionally substituted phenyl. In some embodiments, R is phenyl.

In some embodiments, X⁵ is —C(R^{B5})₂—. In some embodiments, X⁵ is —CHR^{B5}—. In some embodiments, X⁵ is optionally substituted —CH₂—. In some embodiments, X⁵ is —CH₂—. In some embodiments, X⁵ is —N(R^{B5})—. In some embodiments, X⁵ is optionally substituted —NH—. In some embodiments, X⁵ is —NH—. In some embodiments, X⁵ is —C(R^{B5})═. In some embodiments, X⁵ is —C(R)═. In some embodiments, X⁵ is optionally substituted —CH═. In some embodiments, X⁵ is —CH═. In some embodiments, X⁵ is —N═. In some embodiments, X⁵ is —C(O)—. In some embodiments, X⁵ is —C(S)—. In some embodiments, X⁵ is —C(Se)—.

In some embodiments, R^{B5} is halogen. In some embodiments, R^{B5} is -L^{B5}-R^{B51}. In some embodiments, R^{B5} is -L^{B5}-R^{B51}, wherein R^{B51} is R′, —NHR′, —OH, or —SH. In some embodiments, R^{B5} is -L^{B5}-R^{B51}, wherein R^{B51} is —NHR, —OH, or —SH. In some embodiments, R^{B5} is -L^{B5}-R^{B51}, wherein R^{B51} is —NH₂, —OH, or —SH. In some embodiments, R^{B5} is —C(O)—R^{B51}. In some embodiments, R^{B5} is R′. In some embodiments, R^{B5} is R. In some embodiments, R^{B5} is —H. In some embodiments, R^{B5} is —OH. In some embodiments, R^{B5} is —CH₂OH. In some embodiments, R^{B5} is halogen. In some embodiments, R^{B5} is —F. In some embodiments, R^{B5} is —Cl. In some embodiments, R^{B5} is —Br. In some embodiments, R^{B5} is —I. In some embodiments, R^{B5} is —CN. In some embodiments, R^{B5} is —NO₂. In some embodiments, R^{B5} is -L^{B5}-R′.

In some embodiments, when X⁴ is —C(O)—, X⁵ is —C(R^{B5})₂—, —C(R^{B5}) or —N(R^{B5})—, wherein R^{B5} is -L^{B5}-R^{B51}, wherein R^{B51} is —NHR′, —OH, or —SH. In some embodiments, X⁴ is —C(O)—, and R^{B51} is or comprises a hydrogen bond donor, which forms a hydrogen bond with the O of X⁴.

In some embodiments, L^{B5} is a covalent bond. In some embodiments, L^{B5} is or comprises —C(O)—. In some embodiments, L^{B5} is or comprises —O—. In some embodiments, L^{B5} is or comprises —OC(O)—. In some embodiments, L^{B5} is or comprises —CH₂OC(O)—.

In some embodiments, R⁵¹ is —R′. In some embodiments, R⁵¹ is —R. In some embodiments, R⁵¹ is —H. In some embodiments, R⁵¹ is —N(R′)₂. In some embodiments, R is —NHR′. In some embodiments, R⁵¹ is —NHR. In some embodiments, R⁵¹ is —NH₂. In some embodiments, R⁵¹ is —OR′. In some embodiments, R⁵¹ is —OR. In some embodiments, R⁵¹ is —OH. In some embodiments, R⁵¹ is —SR′. In some embodiments, R⁵¹ is —SR. In some embodiments, R⁵¹ is —SH. In some embodiments, R is benzyl. In some embodiments, R is optionally substituted phenyl. In some embodiments, R is phenyl. In some embodiments, R is methyl.

In some embodiments, R^{B5} is —C(O)—R^{B51}. In some embodiments, R^{B5} is —C(O)NHCH₂Ph. In some embodiments, R^{B5} is —C(O)NHPh. In some embodiments, R^{B5} is —C(O)NHCH₃. In some embodiments, R^{B5} is —OC(O)—R^{B51}. In some embodiments, R^{B5} is —OC(O)—R. In some embodiments, R^{B5} is —OC(O)CH₃.

In some embodiments, X⁵ is directly bonded to X¹, and Ring BA is 5-membered.

In some embodiments, X⁶ is —C(R^{B6})═. In some embodiments, X⁶ is optionally substituted —CH═. In some embodiments, X⁶ is —CH═. In some embodiments, X⁶ is —C(OR^{B6})═. In some embodiments, X⁶ is —C(R^{B6})₂—. In some embodiments, X⁶ is optionally substituted —CH₂—. In some embodiments, X⁶ is —CH₂—. In some embodiments, X⁶ is —C(O)—. In some embodiments, X⁶ is —C(S)—. In some embodiments, X⁶ is —C(Se)—. In some embodiments, X⁶ is —N═.

In some embodiments, R^{B6} is -L^{B6}- R^{B61}. In some embodiments, two R^{B6} on the same atom are taken together to form ═O, ═C(-L^{B6}-R^{B61})₂, ═N-L^{B6}- R^{B61}, or optionally substituted ═CH₂ or ═NH. In some embodiments, two R^{B6} on the same atom are taken together to form ═O. In some embodiments, L^{B6} is a covalent bond. In some embodiments, R^{B6} is R. In some embodiments, R^{B6} is —H.

In some embodiments, R^{B6} is a protecting group, e.g., an amino or hydroxyl protecting group suitable for oligonucleotide synthesis. In some embodiments, R^{B6} is R. In some embodiments, R^{B6} is —H. In some embodiments, R^{B6} is halogen. In some embodiments, R^{B6} is —F. In some embodiments, R^{B6} is —Cl. In some embodiments, R^{B6} is —Br. In some embodiments, R^{B6} is —I. In some embodiments, R^{B6} is —CN. In some embodiments, R^{B6} is —NO₂. In some embodiments, R^{B6} is -L^{B6}-R′.

In some embodiments, L^{B6} is a covalent bond. In some embodiments, L^{B6} is optionally substituted C₁₋₁₀alkylene. In some embodiments, L^{B6} is —CH₂CH₂—. In some embodiments, R^{B6} is —CH₂CH₂-(4-nitrophenyl).

In some embodiments, R^{B61} is R′. In some embodiments, R^{B61} is R. In some embodiments, R^{B61} is —H.

In some embodiments, Ring BA^{A} is monocyclic. In some embodiments, Ring BA^{A} is 5-membered. In some embodiments, Ring BA^{A} is 6-membered. In some embodiments, Ring BA^{A} is bicyclic. In some embodiments, Ring BA^{A} is 9-membered. In some embodiments, Ring BA^{A} is 10-membered. In some embodiments, Ring BA^{A} has one heteroatom. In some embodiments, Ring BA^{A} has 2 heteroatoms. In some embodiments, Ring BA^{A} has 3 heteroatoms. In some embodiments, Ring BA^{A} has 4 heteroatoms. In some embodiments, Ring BA^{A} has 5 heteroatoms. In some embodiments, a heteroatom is nitrogen. In some embodiments, a heteroatom is oxygen.

In some embodiments, X^{1′} is —(N−)−. In some embodiments, X^{1′} is —C(−)═.

In some embodiments, X²′ is —C(O)—. In some embodiments, X^{2′} is —C(S)—. In some embodiments, X^{2′} is —C(Se)—. In some embodiments, X²′ is —C(R^{B2}′)═. In some embodiments, X^{2′} is optionally substituted —CH₂—. In some embodiments, X² is —CH₂—. In some embodiments, X^{2′} is optionally substituted —CH═. In some embodiments, X^{2′} is —CH═. In some embodiments, X” is —C(OR^{B2}′)═. In some embodiments, X²′ is —C(OR^{B2}′)=, wherein R^{B2} is -L^{B2}‘-R’. In some embodiments, X^{2′} is —N═.

In some embodiments, L^{B2}′ is a covalent bond.

In some embodiments, R^{B2}′ is R′. In some embodiments, R^{B2} is R. In some embodiments, R^{B2} is not —H. In some embodiments, R^{B2} is —H. In some embodiments, R^{B2} is halogen. In some embodiments, R^{B2}is —F. In some embodiments, R^{B2}′ is —Cl. In some embodiments, R^{B2}′ is —Br. In some embodiments, R^{B2} is —I. In some embodiments, R^{B2}′ is —CN. In some embodiments, R^{B2}′ is —NO₂. In some embodiments, R^{B2} is -L^{B2}′-R′.

In some embodiments, X³ is —N(R^{B3}′)—. In some embodiments, X^{3′} is —N(R^{B3}′)—, wherein R^{B3} is -L^{B3}′-R′. In some embodiments, X^{3′} is —N(R′)—. In some embodiments, X^{3′} is optionally substituted —NH—. In some embodiments, X³ is —NH—. In some embodiments, X^{3′} is —N═. In some embodiments, X³′ is —C(R^{B3}′)═.

In some embodiments, L^{B3}′ is a covalent bond.

In some embodiments, R^{B3}′ is R′. In some embodiments, R^{B3}′ is R. In some embodiments, R^{B3}′ is —H. In some embodiments, R^{B3} is not —H. In some embodiments, R^{B3}′ is halogen. In some embodiments, R^{B3}′ is —F. In some embodiments, R^{B3}′ is —Cl. In some embodiments, R^{B3} is —Br. In some embodiments, R^{B3}′ is —I. In some embodiments, R^{B3}′ is —CN. In some embodiments, R^{B3}′ is —NO₂. In some embodiments, R^{B3} is -L^{B}′-R′.

In some embodiments, X⁴′ is —C(R^{B4}′)═. In some embodiments, X⁴′ is optionally substituted —CH═. In some embodiments, X⁴′ is —CH═. In some embodiments, X⁴′ is —C(OR^{B4}′)═. In some embodiments, X⁴ is —C(OR^{B4}′)═, wherein R^{B4}′ is -L^{B4}′-R^{B41}′. In some embodiments, X⁴′ is —C(—N(R^{B4}′)₂)═. In some embodiments, X⁴′ is —C(—NHR^{B4}′)═. In some embodiments, X⁴′ is optionally substituted —C(—NH₂)═. In some embodiments, X⁴′ is —C(—NH₂)═. In some embodiments, X⁴′ is —C(—NHR′)═. In some embodiments, X⁴′ is —C(—N(R′)₂)═. In some embodiments, X⁴′ is —C(—NHC(O)R′)═. In some embodiments, X⁴′ is —C(—NHC(O)NHR′)═. In some embodiments, X⁴′ is —C(—NHC(O)N(R′)₂)═. In some embodiments, X⁴′ is —C(—N(R′)C(O)N(R′)₂)═. In some embodiments, R′ is —H. In some embodiments, R′ is optionally substituted C₁₋₆ aliphatic. In some embodiments, R′ is optionally substituted C₁₋₆ alkyl. In some embodiments, R′ is C₁₋₆ alkyl. In some embodiments, R′ is methyl. In some embodiments, R′ is ethyl. In some embodiments, R′ is isopropyl. In some embodiments, R′ is optionally substituted phenyl. In some embodiments, R′ is phenyl. In some embodiments, R′ is optionally substituted naphthyl. In some embodiments, R′ is optionally substituted 2-naphthyl. In some embodiments, R′ is 2-naphthyl. In some embodiments, R′ is C₁₋₆ alkyl. In some embodiments, X⁴′ is —C(—NH₂)═. In some embodiments, X⁴′ is —C(—N(CH₃)₂)═. In some embodiments, X⁴′ is —C(—NHEt)═. In some embodiments, X⁴′ is —C(—NH(i-Pr))═. In some embodiments, X⁴′ is —C(—NHC(O)Ph)═. In some embodiments. X⁴′ is —C(—NHC(O)NHCH₃)═. In some embodiments, X⁴′ is —C(—NHC(O)NHPh)═. In some embodiments, X⁴′ is —C(—NHC(O)NH(2-naphthyl))=. In some embodiments, X⁴′ is —C(R^{B4}′)₂—. In some embodiments, X⁴′ is optionally substituted —CH₂—. In some embodiments, X⁴′ is —CH₂—. In some embodiments, X⁴′ is —C(O)—. In some embodiments, X⁴′ is —C(S)—. In some embodiments, X⁴′ is —C(Se)—. In some embodiments, X⁴′ is —C(═NR^{B4}′)—. In some embodiments, X⁴′ is —N(R^{B4}′). In some embodiments, X⁴′ is optionally substituted —NH—. In some embodiments, X⁴′ is —NH—. In some embodiments, X⁴ is —N═.

In some embodiments, R^{B4}′ is -L^{B4}′-R^{B41}′. In some embodiments, two R^{B4}′ on the same atom are taken together to form ═O, ═C(-L^{B4}′-R^{B41}′)₂, ═N-L^{B4}′-R^{B41}′, or optionally substituted ═CH₂ or ═NH. In some embodiments, two R^{B4}′ on the same atom are taken together to form ═O. In some embodiments, two R^{B4}′ on the same atom are taken together to form ═C(-L^{B4}′-R^{B41}′)₂. In some embodiments, two R^{B4}′ on the same atom are taken together to form ═N-L^{B4}′-R^{B41}′. In some embodiments, two R^{B4}′ on the same atom are taken together to form ═CH₂. In some embodiments, two R^{B4}′ on the same atom are taken together to form ═NH. In some embodiments, a formed group is a suitable protecting group, e.g., amino protecting group, for oligonucleotide synthesis.

In some embodiments, X⁴′ is —C(—N═C(-L^{B4}′-R^{B41}′)₂)═. In some embodiments, X⁴ is C(N═CHL^{B4}′R^{B41}′)═. In some embodiments, X⁴ is —C(—N═CH—N(CH₃)₂)═.

In some embodiments, R^{B4}′ is R′. In some embodiments, R^{B4}′ is R. In some embodiments, R^{B4}′ is —H. In some embodiments, R^{B4}′ is not —H. In some embodiments, R^{B4}′ is halogen. In some embodiments, R^{B4}is —F. In some embodiments, R^{B4}′ is —Cl. In some embodiments, R^{B4}′ is —Br. In some embodiments, R^{B4} is —I. In some embodiments, R^{B4}′ is —CN. In some embodiments, R^{B4}′ is —NO₂. In some embodiments, R^{B4} is -L^{B4}′-R′.

In some embodiments, R^{B4}′ is a protecting group, e.g., an amino or hydroxyl protecting group suitable for oligonucleotide synthesis. In some embodiments, R^{B4}′ is R. In some embodiments, R^{B4}′ is —CH₂CH₂-(4-nitrophenyl).

In some embodiments, L^{B4}′ is a covalent bond. In some embodiments, L^{B4}′ is optionally substituted C₁₋₁₀ alkylene. In some embodiments, L^{B4}′ is —CH₂CH₂—. In some embodiments, at least one methylene unit is replaced with —N(R′)—. In some embodiments, R′ is R. In some embodiments, R is optionally substituted phenyl. In some embodiments, R is phenyl. In some embodiments, R is methyl. In some embodiments, R is —H.

In some embodiments, R^{B41}′ is R′. In some embodiments, R^{B41′} is R. In some embodiments, R^{B41}′ is —H.

In some embodiments, X⁵′ is —C(R^{B5}′)₂—. In some embodiments, X^{5′} is optionally substituted —CH₂—. In some embodiments, X^{5′} is —CH₂—. In some embodiments, X⁵′ is —N(R^{B5}′). In some embodiments, X^{5′} is optionally substituted —NH—. In some embodiments, X^{5′} is —NH—. In some embodiments, X^{5′} is —C(R^{B5}′)═. In some embodiments, X^{5′} is optionally substituted —CH═. In some embodiments, X⁵ is —CH═. In some embodiments, X^{5′} is —C(O)—. In some embodiments, X⁵ is —C(S)—. In some embodiments, X^{5′} is —C(Se)—. In some embodiments, X^{5′} is —N═.

In some embodiments, L^{B5}′ is a covalent bond.

In some embodiments, R^{B5}′ is R′. In some embodiments, R^{B5}′ is R. In some embodiments, R^{B5} is —H. In some embodiments, R^{B5}′ is not —H.

In some embodiments, R^{B5}′ is halogen. In some embodiments, R^{B5}′ is —F. In some embodiments, R^{B5}′ is —Cl. In some embodiments, R^{B5}′ is —Br. In some embodiments, R^{B5}′ is —I. In some embodiments, R^{B5}′ is —CN. In some embodiments, R^{B5}′ is —NO₂. In some embodiments, R^{B5}′ is -L^{B5}′-R′.

In some embodiments, X^{6′} is —C(R^{B6}′)═. In some embodiments, X^{6′} is optionally substituted —CH═. In some embodiments, X⁶′ is —CH═. In some embodiments, X^{6′} is —C(OR^{B6}′)═. In some embodiments, X⁶′ is —C(OR^{B6}′)═, wherein R^{B6′} is -L^{B6}′-R^{B61}′. In some embodiments, X⁶′ is —C(R^{B6}′)₂—. In some embodiments, X^{6′} is optionally substituted —CH₂—. In some embodiments, X^{6′} is —CH₂—. In some embodiments, X^{6′} is —C(O)—. In some embodiments, X^{6′} is —C(S)—. In some embodiments, X^{6′} is —C(Se)—. In some embodiments, X^{6′} is —C(—N(R^{B6}′)₂)═. In some embodiments, X^{6′} is —C(—N(R^{B6}′)₂)═, wherein each R^{B6}′ is independently -L^{B6}′-R^{B61}′. In some embodiments, X⁶′ is —C(—N(R′)₂)═. In some embodiments, X⁶′ is —C(—NHR′)═. In some embodiments, X⁶′ is —C(—NHC(O)R)═. In some embodiments, X^{6′} is —N═.

In some embodiments, R^{B6}′ is -L^{B6}′R^{B61}′. In some embodiments, two R^{B6}′ on the same atom are taken together to form ═O, ═C(-L^{B6}′R^{B61}′)₂, ═N-L^{B6}′R^{B61}′, or optionally substituted ═CH₂ or ═NH. In some embodiments, two R^{B6}′ on the same atom are taken together to form ═O.

In some embodiments, L^{B6}′ is a covalent bond. In some embodiments, L^{B6}′ is optionally substituted C₁₋₁₀ alkylene. In some embodiments, L^{B6}′ is —CH₂CH₂—.

In some embodiments, R^{B6}′ is R′. In some embodiments, R^{B6}′ is R. In some embodiments, R^{B6}′ is —H. In some embodiments, R^{B6}′ is not —H. In some embodiments, R^{B6}′ is a protecting group, e.g., an amino or hydroxyl protecting group suitable for oligonucleotide synthesis. In some embodiments, R^{B6}′ is R′. In some embodiments, R^{B6}′ is —CH₂CH₂-(4-nitrophenyl).

In some embodiments, R^{B61}′ is R′. In some embodiments, R^{B61}′ is R. In some embodiments, R^{B61}′ is —H. In some embodiments, R^{B61}′ is not —H.

In some embodiments, R^{B6}′ is halogen. In some embodiments, R^{B6}′ is —F. In some embodiments, R^{B6}′ is —Cl. In some embodiments, R^{B6}′ is —Br. In some embodiments, R^{B6}′ is —I. In some embodiments, R^{B6}′ is —CN. In some embodiments, R^{B6}′ is —NO₂. In some embodiments, R^{B6} is -L^{B6}′-R′.

In some embodiments, X⁷′ is —C(R^{B7}′)═. In some embodiments, X⁷′ is optionally substituted —CH═. In some embodiments, X⁷′ is —CH═. In some embodiments, X^{7′} is —C(OR^{B7}′)═. In some embodiments, X⁷′ is —C(OR^{B7}′)═, wherein R^{B7}′ is -L^{B7}′-R^{B71}′. In some embodiments, X^{7′} is —C(R^{B7}′)₂—. In some embodiments, X⁷′ is optionally substituted —CH₂—. In some embodiments, X^{7′} is —CH₂—. In some embodiments, X⁷′ is —C(O)—. In some embodiments, X⁷′ is —C(S)—. In some embodiments, X⁷′ is —C(Se)—. In some embodiments, X⁷′ is —C(—N(R^{D7}′)₂)═. In some embodiments, X⁷′ is —C(—N(R^{B7}′)₂)═, wherein each R^{B7}′ is independently -L^{B7}′-R^{B71}′. In some embodiments, X⁷′ is —N(R^{B7}′)—. In some embodiments, X^{7′} is optionally substituted —NH—. In some embodiments, X^{7′} is —NH—. In some embodiments, X^{7′} is —N═.

In some embodiments, R^{B7}′ is -L^{B7}′-R^{B71}′. In some embodiments, two R^{B7}′ on the same atom are taken together to form ═O, ═C(-L^{B7}′-R^{B71}′)₂, ═N-L^{B7}′R^{B71}′ or optionally substituted ═CH₂ or ═NH. In some embodiments, two R^{B7}′ on the same atom are taken together to form ═O. In some embodiments, L^{B7}′ is a covalent bond. In some embodiments, R^{B7}′ is R. In some embodiments, R^{B7}′ is —H. In some embodiments, R^{B7}′ is not —H. In some embodiments, R^{B7}′ is halogen. In some embodiments, R^{B7}′ is —F. In some embodiments, R^{B7}′ is —Cl. In some embodiments, R^{B7}′ is —Br. In some embodiments, R^{B7}′ is —I. In some embodiments, R^{B7}′ is —CN. In some embodiments, R^{B7}′ is —NO₂. In some embodiments, R^{B7}′ is -L^{B7}′R′.

In some embodiments, L^{B7}′ is a covalent bond. In some embodiments, L^{B7}′ is optionally substituted C₁₋₁₀ alkylene. In some embodiments, L^{B7}′ is —CH₂CH₂—.

In some embodiments, R^{B71}′ is R′. In some embodiments, R^{B71}′ is R. In some embodiments, R^{B71}′ is —H. In some embodiments, R^{B71}′ is not —H.

In some embodiments, L^{B} is a covalent bond. In some embodiments, L^{B} is an optionally substituted bivalent C₁₋₁₀ saturated or partially unsaturated aliphatic chain, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{B} is an optionally substituted bivalent C₁₋₁₀ saturated or partially unsaturated heteroaliphatic chain having 1-6 heteroatoms, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, at least methylene unit is replaced. In some embodiments, L^{B} is optionally substituted C₁₋₁₀ alkylene. In some embodiments, L^{B} is optionally substituted C₁₋₆ alkylene. In some embodiments, L^{B} is optionally substituted C₁₋₄ alkylene. In some embodiments, L^{B} is —CH₂CH₂—. In some embodiments, at least one methylene unit is replaced with —C(O)—. In some embodiments, at least one methylene unit is replaced with —C(O)N(R′)—. In some embodiments, at least one methylene unit is replaced with —N(R′)—. In some embodiments, at least one methylene unit is replaced with —NH—. In some embodiments, at least one methylene unit is replaced with —Cy—. In some embodiments, L^{B} is or comprises optionally substituted —N═CH—. In some embodiments, L^{B}is or comprises —C(O)—. In some embodiments, L^{B} is or comprises —O—. In some embodiments, L^{B} is or comprises —OC(O)—. In some embodiments, L^{B} is or comprises —CH₂OC(O)—.

In some embodiments, each —Cy— is independently an optionally substituted, 3-20 membered, monocyclic, bicyclic or polycyclic, saturated, partially saturated or aromatic ring having 0-10 heteroatoms. Suitable monocyclic unit(s) of —Cy— are described herein. In some embodiments, —Cy— is monocyclic. In some embodiments, —Cy— is bicyclic. In some embodiments, —Cy— is polycyclic. In some embodiments, —Cy-is an optionally substituted bivalent 3-10 membered monocyclic, saturated or partially unsaturated ring having 0-5 heteroatoms. In some embodiments, —Cy— is an optionally substituted bivalent 5-10 membered aromatic ring having 0-5 heteroatoms. In some embodiments, —Cy— is optionally substituted phenylene. In some embodiments, —Cy— is phenylene.

In some embodiments, R′ is R. In some embodiments, R′ is —C(O)R. In some embodiments, R′ is —C(O)OR. In some embodiments, R′ is —C(O)N(R)₂. In some embodiments, R′ is —SO₂R.

In some embodiments, R′ in various structures is a protecting group (e.g., for amino, hydroxyl, etc.), e.g., one suitable for oligonucleotide synthesis. In some embodiments, R is optionally substituted phenyl. In some embodiments, R is phenyl. In some embodiments, R is 4-nitrophenyl. In some embodiments, R is —CH₂CH₂-(4-nitrophenyl). In some embodiments, R′ is —C(O)NPh₂.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₂₀ arylaliphatic, C₆₋₂₀ arylheteroaliphatic having 1-5 heteroatoms, 5-14 membered heteroaryl having 1-5 heteroatoms, and 3-10 membered heterocyclyl having 1-5 heteroatoms, or
two R groups are optionally and independently taken together to form a covalent bond, or:
two or more R groups on the same atom are optionally and independently taken together with the atom to form an optionally substituted, 3-15 membered, monocyclic, bicyclic or polycyclic ring having, in addition to the atom, 0-10 (e.g., 0, 1, 2, 3, 4, 5, 6, 7, 8, 9, or 10) heteroatoms; or:
two or more R groups on two or more atoms are optionally and independently taken together with their intervening atoms to form an optionally substituted, 3-15 membered, monocyclic, bicyclic or polycyclic ring having, in addition to the intervening atoms, 0-10 (e.g., 0, 1, 2, 3, 4, 5, 6, 7, 8, 9, or 10) heteroatoms.

In some embodiments, each R is independently —H, or an optionally substituted group selected from C₁₋₂₀ aliphatic, C₁₋₂₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-20 membered heteroaryl having 1-10 heteroatoms, and 3-30 membered heterocyclyl having 1-10 heteroatoms. In some embodiments, each R is independently —H, or an optionally substituted group selected from C₁₋₁₀ aliphatic, C₁-10 heteroaliphatic having 1-5 heteroatoms, C₆₋₁₄ aryl, C₆₋₂₀ arylaliphatic, C₆₋₂₀ arylheteroaliphatic having 1-5 heteroatoms, 5-14 membered heteroaryl having 1-5 heteroatoms, and 3-10 membered heterocyclyl having 1-5 heteroatoms. In some embodiments, two R groups are optionally and independently taken together to form a covalent bond. In some embodiments, two or more R groups on the same atom are optionally and independently taken together with the atom to form an optionally substituted, 3-20 membered, monocyclic, bicyclic or polycyclic ring having, in addition to the atom, 0-10 heteroatoms. In some embodiments, two groups on the same atom are optionally and independently taken together with the atom to form an optionally substituted, 3-20 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, two groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, a formed ring is monocyclic. In some embodiments, a formed ring is bicyclic. In some embodiments, a formed ring is polycyclic. In some embodiments, each monocyclic ring unit is independently 3-10 (e.g., 3-8, 3-7, 3-6, 5-10, 5-8, 5-7, 5-6, 3, 4, 5, 6, 7, 8, 9, or 10, etc.) membered, and is independently saturated, partially saturated, or aromatic, and independently has 0-5 heteroatom. In some embodiments, a ring is saturated. In some embodiments, a ring is partially saturated. In some embodiments, a ring is aromatic. In some embodiments, a formed ring has 1-5 heteroatom. In some embodiments, a formed ring has 1 heteroatom. In some embodiments, a formed ring has 2 heteroatoms. In some embodiments, a heteroatom is nitrogen. In some embodiments, a heteroatom is oxygen.

In some embodiments, R is —H.

In some embodiments, R is optionally substituted C₁₋₂₀, C₁₋₁₅, C₁₋₁₀, C₁₋₈, C₁₋₆, C₁₋₅, C₁₋₄, C₁₋₃, or C₁₋₂ aliphatic. In some embodiments, R is optionally substituted alkyl. In some embodiments, R is optionally substituted C₁₋₆ alkyl. In some embodiments, R is optionally substituted methyl. In some embodiments, R is optionally substituted cycloaliphatic. In some embodiments, R is optionally substituted cycloalkyl.

In some embodiments, R is optionally substituted C₁₋₂₀ heteroaliphatic having 1-10 heteroatoms.

In some embodiments, R is optionally substituted C₆₋₂₀ aryl. In some embodiments, R is optionally substituted phenyl. In some embodiments, R is phenyl.

In some embodiments, R is optionally substituted C₆₋₂₀ arylaliphatic. In some embodiments, R is optionally substituted C₆₋₂₀ arylalkyl. In some embodiments, R is benzyl. In some embodiments, R is optionally substituted C₆-20 arylheteroaliphatic having 1-10 heteroatoms.

In some embodiments, R is optionally substituted 5-20 membered heteroaryl having 1-10 heteroatoms. In some embodiments, R is optionally substituted 5-membered heteroaryl having 1-4 heteroatoms. In some embodiments, R is optionally substituted 6-membered heteroaryl having 1-4 heteroatoms. In some embodiments, R is optionally substituted 3-20 membered heterocyclyl having 1-10 heteroatoms. In some embodiments, R is optionally substituted 3-10 membered heterocyclyl having 1-5 heteroatoms. In some embodiments, R is optionally substituted 5-6 membered heterocyclyl having 1-5 heteroatoms. In some embodiments, a heterocyclyl is saturated. In some embodiments, a heterocyclyl is partially saturated.

In some embodiments, a heteroatom is selected from boron, nitrogen, oxygen, sulfur, silicon and phosphorus. In some embodiments, a heteroatom is selected from nitrogen, oxygen, sulfur, and silicon. In some embodiments, a heteroatom is selected from nitrogen, oxygen, and sulfur. In some embodiments, a heteroatom is nitrogen. In some embodiments, a heteroatom is oxygen. In some embodiments, a heteroatom is sulfur.

As described herein, various groups may be optionally substituted. Substituents are routinely utilized in chemistry including in development of various therapeutics. Many substituents can be utilized in accordance with the present disclosure. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is no more than about 2; in some embodiments, it is no more than about 3; in some embodiments, it is no more than about 4; in some embodiments, it is no more than about 5; in some embodiments, it is no more than about 6; in some embodiments, it is no more than about 7; in some embodiments, it is no more than about 8; in some embodiments, it is no more than about 9; in some embodiments, it is no more than about 10; in some embodiments, it is no more than about 11; in some embodiments, it is no more than about 12; in some embodiments, it is no more than about 13; in some embodiments, it is no more than about 14; in some embodiments, it is no more than about 15.

As appreciated by those skilled in the art, embodiments described for variables can be readily combined to provide various structures. Those skilled in the art also appreciates that embodiments described for a variable can be readily utilized for other variables that can be that variable, e.g., embodiments of R for R′ R^{B2}, R^{B3}, R^{B4}, R^{B5}, R^{B6}, R^{B2}′, R^{B3}′, R^{B4}′, R^{B5}′, R^{B6}′, etc.; embodiments of embodiments of L^{B} for L^{B2}, L^{B3}, L^{B4}, L^{B5}, L^{B6}, L^{B2}′, L^{B3}′, L^{B4}′, L^{B5}′, L^{B6}′, etc. Exemplary embodiments and combinations thereof include but are not limited to structures exemplified herein. Certain examples are described below.

For example, in some embodiments, Ring BA is optionally substituted or protected

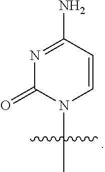 [SMILES: *n1ccc(N)nc1=O]

In some embodiments, Ring BA is

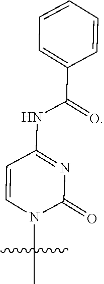 [SMILES: *n1ccc(NC(=O)c2ccccc2)nc1=O]

In some embodiments. Ring BA is

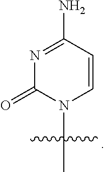 [SMILES: *n1ccc(N)nc1=O]

In some embodiments, Ring BA is optionally substituted or protected

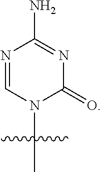 [SMILES: *n1cnc(N)nc1=O]

In some embodiments, Ring BA is

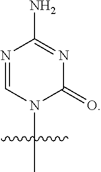 [SMILES: *n1cnc(N)nc1=O]

In some embodiments, X⁴ is —C(O)—, and O in —C(O)— of X⁴ may form a hydrogen bond with a —H of R⁵, e.g., a —H in —NHR′, —OH, or —SH of R⁵′. In some embodiments, X⁴ is —C(O)—, and X⁵ is —C(R⁵)═. In some embodiments, R⁵′ is —NHR′. In some embodiments, R⁵ is -L^{B5}-NHR′. In some embodiments, L^{B5} is optionally substituted —CH₂—. In some embodiments, a methylene unit is replaced with —C(O)—. In some embodiments, L^{B5} is —C(O)—. In some embodiments, R′ is optionally substituted methyl. In some embodiments, R′ is —CH₂Ph. In some embodiments, R′ is optionally substituted phenyl. In some embodiments, R′ is phenyl. In some embodiments, R′ is optionally substituted C₁₋₆ aliphatic. In some embodiments, R′ is optionally substituted C₁₋₆ alkyl. In some embodiments, R′ is optionally substituted methyl. In some embodiments, R′ is methyl. In some embodiments, Ring BA is optionally protected

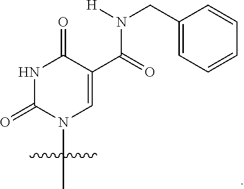 [SMILES: *n1cc(C(=O)NCc2ccccc2)c(=O)[nH]c1=O]

In some embodiments, Ring BA is

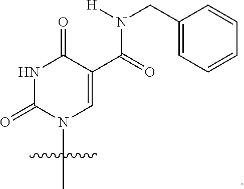 [SMILES: *n1cc(C(=O)NCc2ccccc2)c(=O)[nH]c1=O]

In some embodiments, Ring BA is optionally protected

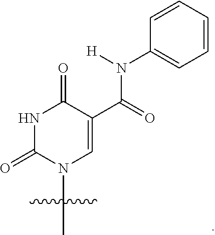 [SMILES: *n1cc(C(=O)Nc2ccccc2)c(=O)[nH]c1=O]

In some embodiments, Ring BA is

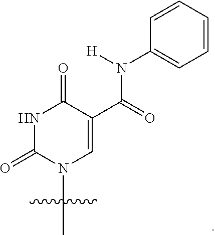 [SMILES: *n1cc(C(=O)Nc2ccccc2)c(=O)[nH]c1=O]

In some embodiments, Ring BA is optionally protected

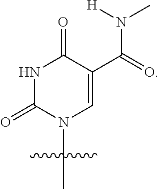 [SMILES: *n1cc(C(=O)NC)c(=O)[nH]c1=O]

In some embodiments, Ring BA is

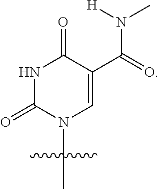 [SMILES: *n1cc(C(=O)NC)c(=O)[nH]c1=O]

In some embodiments, Ring BA is optionally protected

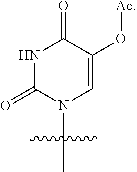 [SMILES: *n1cc(OC(C)=O)c(=O)[nH]c1=O]

In some embodiments, Ring BA is

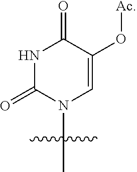 [SMILES: *n1cc(OC(C)=O)c(=O)[nH]c1=O]

In some embodiments, Ring BA is optionally protected

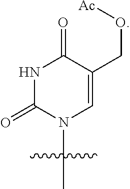 [SMILES: *n1cc(COC(C)=O)c(=O)[nH]c1=O]

In some embodiments, Ring BA is

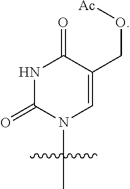 [SMILES: *n1cc(COC(C)=O)c(=O)[nH]c1=O]

In some embodiments, Ring BA is optionally protected

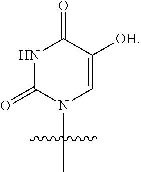 [SMILES: *n1cc(O)c(=O)[nH]c1=O]

In some embodiments, Ring BA is

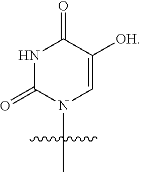 [SMILES: *n1cc(O)c(=O)[nH]c1=O]

In some embodiments, Ring BA is optionally protected

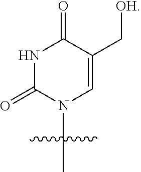 [SMILES: *n1cc(CO)c(=O)[nH]c1=O]

In some embodiments, Ring BA is

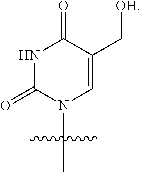 [SMILES: *n1cc(CO)c(=O)[nH]c1=O]

In some embodiments, X¹ is —C(−)═, and X⁴ is ═C(—N(R^{B4})₂)—. In some embodiments, two R groups on the same atom, e.g., a nitrogen atom, are taken together to form optionally substituted ═CH₂ or ═NH. In some embodiments, two R groups on the same atom, e.g., a nitrogen atom, are taken together to form optionally substituted ═C(-L^{B4}-R)₂, ═N-L^{B4}-R. In some embodiments, a formed group is ═CHN(R)₂. In some embodiments, a formed group is ═CHN(CH₃)₂. In some embodiments, X⁴ is ═C(—N═CHN(CH₃)₂)—. In some embodiments, —N(R^{B4})₂ is —NR^{B4}. In some embodiments, R^{B4} is —NHC(O)R. In some embodiments, Ring BA is optionally substituted or protected

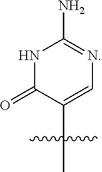 [SMILES: *c1cnc(N)[nH]c1=O]

In some embodiments, Ring BA is

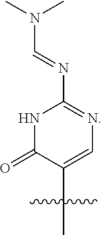 [SMILES: *c1cnc(/N=C/N(C)C)[nH]c1=O]

In some embodiments, Ring BA is

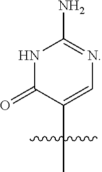 [SMILES: *c1cnc(N)[nH]c1=O]

In some embodiments, Ring BA is optionally substituted

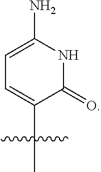 [SMILES: *c1ccc(N)[nH]c1=O]

In some embodiments, Ring BA is optionally protected

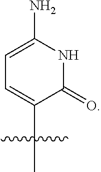 [SMILES: *c1ccc(N)[nH]c1=O]

In some embodiments, Ring BA is

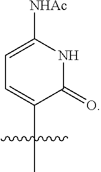 [SMILES: *c1ccc(NC(C)=O)[nH]c1=O]

In some embodiments, Ring BA is

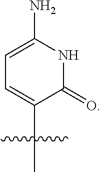 [SMILES: *c1ccc(N)[nH]c1=O]

In some embodiments, X¹ is —N(−)—, X² is —C(O)—, and X³ is —N(R^{B3})—. In some embodiments, X¹ is —N(−)—, X² is —C(O)—, X³ is —N(R^{B3})—, and X⁴ is —C(R^{B4})═. In some embodiments, X¹ is —N(−)—, X² is —C(O)—, X³ is —N(R^{B3})—, X⁴ is —C(R^{B4})═, and X⁵ is —C(R^{B5})═. In some embodiments, Ring BA is optionally substituted or protected

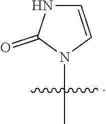 [SMILES: *n1cc[nH]c1=O]

In some embodiments, Ring BA is

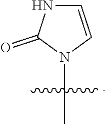 [SMILES: *n1cc[nH]c1=O]

In some embodiments, X³ is —N(R′)—. In some embodiments, R′ is —C(O)R. In some embodiments, X⁴ is —C(R^{B4})₂. In some embodiments, R^{B4} is —R. In some embodiments, R^{B4} is —H. In some embodiments, X⁴ is —CH₂—. In some embodiments, X⁵ is —C(R^{B5})₂—. In some embodiments, R^{B5} is —R. In some embodiments, R^{B5} is —H. In some embodiments, X⁵ is —CH₂—. In some embodiments, Ring BA is optionally substituted or protected

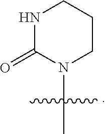 [SMILES: *N1CCCNC1=O]

In some embodiments, Ring BA is

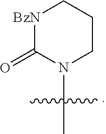

In some embodiments, Ring BA is

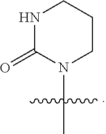 [SMILES: *N1CCCNC1=O]

In some embodiments, X⁴ is —C(R^{B4})═. In some embodiments, X⁴ is —CH═. In some embodiments, X⁵ is —C(R^{B5})═. In some embodiments, X⁵ is —CH═. In some embodiments, Ring BA is optionally substituted or protected

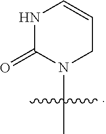 [SMILES: *N1CC=CNC1=O]

In some embodiments, Ring BA

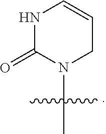 [SMILES: *N1CC=CNC1=O]

In some embodiments, Ring BA is optionally substituted or protected

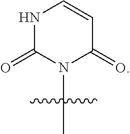 [SMILES: *n1c(=O)cc[nH]c1=O]

In some embodiments, Ring BA is

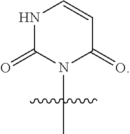 [SMILES: *n1c(=O)cc[nH]c1=O]

In some embodiments, X⁴ is —C(R^{B4})₂—. In some embodiments, X⁴ is —CH₂—. In some embodiments, X⁵ is —C(R^{B5})═. In some embodiments, X⁵ is —CH═. In some embodiments, Ring BA is optionally substituted or protected

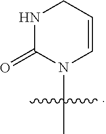 [SMILES: *N1C=CCNC1=O]

In some embodiments, Ring BA is

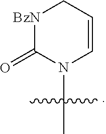

In some embodiments, Ring BA is

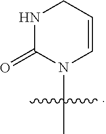 [SMILES: *N1C=CCNC1=O]

In some embodiments, X¹ is —N(−)—, X² is —C(O)—, X³ is —N(R^{B3})—, X⁴ is —C(R^{B4})═, X⁵ is —C(R^{B5})═, X⁶ is —C(O)—. In some embodiments, X₁ is —N(−)—, X² is —C(O)—, X³ is —N(R^{B3})—. X⁴ is —C(R^{B4})═, X⁵ is —C(R^{BS})—, X⁶ is —C(S)—. In some embodiments, each of R^{B3}, R^{B4} and R^{B5} is independently R. In some embodiments, R^{B3} is —H. In some embodiments, R^{B4} is —H. In some embodiments, R^{B5} is —H. In some embodiments, BA is or comprises optionally substituted or protected

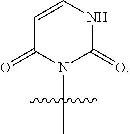 [SMILES: *n1c(=O)cc[nH]c1=O]

In some embodiments, BA is

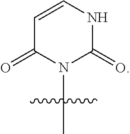 [SMILES: *n1c(=O)cc[nH]c1=O]

In some embodiments, BA is or comprises optionally substituted or protected

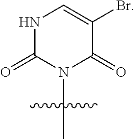 [SMILES: *n1c(=O)[nH]cc(Br)c1=O]

In some embodiments, BA is

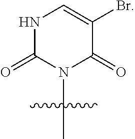 [SMILES: *n1c(=O)[nH]cc(Br)c1=O]

In some embodiments, BA is or comprises optionally substituted or protected

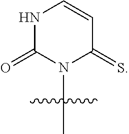 [SMILES: *n1c(=S)cc[nH]c1=O]

In some embodiments, BA is

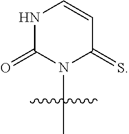 [SMILES: *n1c(=S)cc[nH]c1=O]

In some embodiments, BA is or comprises optionally substituted or protected

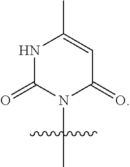 [SMILES: *n1c(=O)cc(C)[nH]c1=O]

In some embodiments, BA is

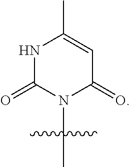 [SMILES: *n1c(=O)cc(C)[nH]c1=O]

In some embodiments, BA is or comprises optionally substituted or protected

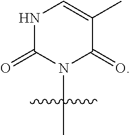 [SMILES: *n1c(=O)[nH]cc(C)c1=O]

In some embodiments, BA is

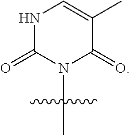 [SMILES: *n1c(=O)[nH]cc(C)c1=O]

In some embodiments, BA is or comprises optionally substituted or protected

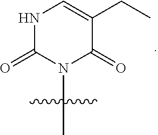 [SMILES: *n1c(=O)[nH]cc(CC)c1=O]

In some embodiments, BA is

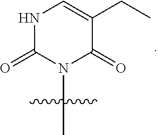 [SMILES: *n1c(=O)[nH]cc(CC)c1=O]

In some embodiments, BA is or comprises optionally substituted or protected

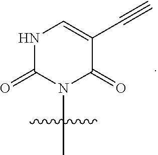 [SMILES: *n1c(=O)[nH]cc(C#C)c1=O]

In some embodiments, BA is

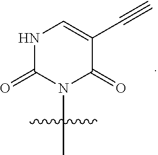 [SMILES: *n1c(=O)[nH]cc(C#C)c1=O]

In some embodiments, BA is or comprises optionally substituted or protected

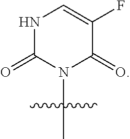 [SMILES: *n1c(=O)[nH]cc(F)c1=O]

In some embodiments, BA is

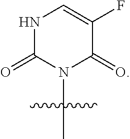 [SMILES: *n1c(=O)[nH]cc(F)c1=O]

In some embodiments, BA is or comprises optionally substituted or protected

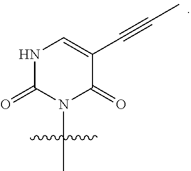 [SMILES: *n1c(=O)[nH]cc(C#CC)c1=O]

In some embodiments, BA is

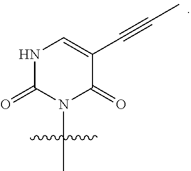 [SMILES: *n1c(=O)[nH]cc(C#CC)c1=O]

In some embodiments, BA is or comprises optionally substituted protected

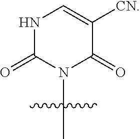 [SMILES: *n1c(=O)[nH]cc(C#N)c1=O]

In some embodiments, BA is

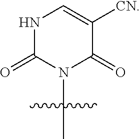 [SMILES: *n1c(=O)[nH]cc(C#N)c1=O]

In some embodiments, BA is or comprises optionally substituted or protected

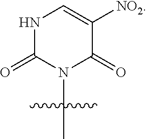 [SMILES: *n1c(=O)[nH]cc([N+](=O)[O-])c1=O]

In some embodiments, BA is

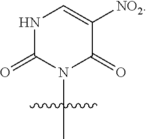 [SMILES: *n1c(=O)[nH]cc([N+](=O)[O-])c1=O]

In some embodiments, X¹ is —N(−)—, X² is —C(O)—, X³ is —N(R^{B3})—. In some embodiments, X⁴ is —C(R^{B4})₂—, wherein the two R^{B4} are taken together to form ═O, or ═C(-L^{B4}-R^{B41})₂, ═N-L^{B4}-R^{B41}. In some embodiments, X⁴ is —C(═NR^{B4})—. In some embodiments, X⁵ is —C(R^{B5})═. In some embodiments, R^{B41} or R^{B4} and R^{B5} are R, and are taken together with their intervening atoms to form an optionally substituted ring as described herein. In some embodiment, Ring BA is optionally substituted or protected

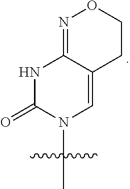 [SMILES: *N1C=C2CCON=C2NC1=O]

In some embodiment, Ring BA is

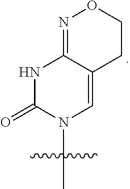 [SMILES: *N1C=C2CCON=C2NC1=O]

In some embodiment, Ring BA is optionally substituted or protected

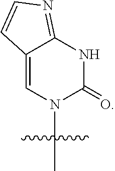 [SMILES: *n1cc2ccnc-2[nH]c1=O]

In some embodiment, Ring BA is

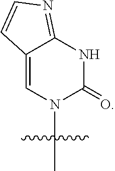 [SMILES: *n1cc2ccnc-2[nH]c1=O]

In some embodiments, X¹ is —N(−)—, X² is —C(O)—, X³ is —N═. In some embodiments, X⁴ is —C(—N(R^{B4})₂)═. In some embodiments, X⁴ is —C(—NHR^{B4})═. In some embodiments, X⁵ is —C(R^{B5})═. In some embodiments, one R^{B4} and R^{B5} are taken together to form an optionally substituted ring as described herein. In some embodiments, a formed ring is an optionally substituted 5-membered ring having a nitrogen atom. In some embodiment, Ring BA is optionally substituted or protected

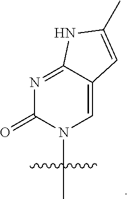 [SMILES: *n1cc2cc(C)[nH]c2nc1=O]

In some embodiment, Ring BA is

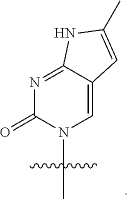 [SMILES: *n1cc2cc(C)[nH]c2nc1=O]

In some embodiment, Ring BA is optionally substituted or protected

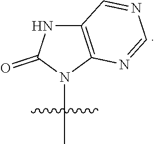 [SMILES: *n1c(=O)[nH]c2cncnc21]

In some embodiment, Ring BA is

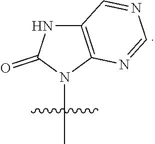 [SMILES: *n1c(=O)[nH]c2cncnc21]

In some embodiment, Ring BA is optionally substituted or protected

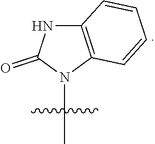 [SMILES: *n1c(=O)[nH]c2ccccc21]

In some embodiment, Ring BA is

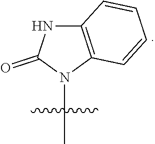 [SMILES: *n1c(=O)[nH]c2ccccc21]

In some embodiment. Ring BA is optionally substituted or protected

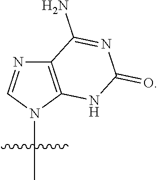 [SMILES: *n1cnc2c(N)nc(=O)[nH]c21]

In some embodiment, Ring BA is

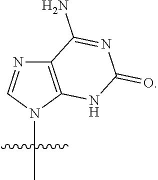 [SMILES: *n1cnc2c(N)nc(=O)[nH]c21]

In some embodiments, Ring BA has the structure of formula BA-IV or BA-V. In some embodiments, X¹ is —N(−)—, X² is —C(O)—, and X³ is —N═. In some embodiments, X¹ is —N(−)—, X² is —C(O)—, X³ is —N═, and X⁶ is —C(R^{B6})═. In some embodiments, Ring BA^{A} is 5-6 membered. In some embodiments, Ring BA^{A} is monocyclic. In some embodiments, Ring BA^{A} is partially unsaturated. In some embodiments, Ring BA^{A} is aromatic. In some embodiments, Ring BA^{A} has 0-2 heteroatoms. In some embodiments, Ring BA^{A} has 1-2 heteroatoms. In some embodiments, Ring BA^{A} has one heteroatom. In some embodiments, Ring BA^{A} has 2 heteroatoms. In some embodiments, a heteroatom is nitrogen. In some embodiments, heteroatom is oxygen. In some embodiments, Ring BA is optionally substituted or protected

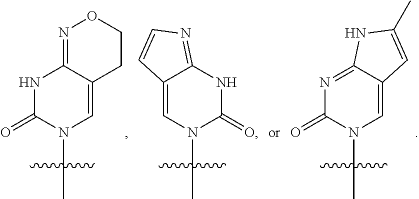

In some embodiments, Ring BA is

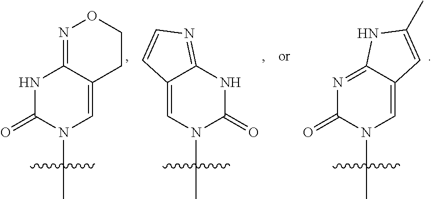

In some embodiments, Ring BA is an optionally substituted 5-membered ring. In some embodiments, X¹ is bonded to X⁵. In some embodiments, each of X⁴ and X⁵ is independently —CH═. In some embodiments, X¹ is —N(−)—. X² is —C(O)—, X³ is —NH—, X⁴ is —CH═, and X⁵ is —CH═. In some embodiments, Ring BA is optionally substituted or protected

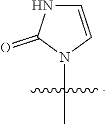 [SMILES: *n1cc[nH]c1=O]

In some embodiments, Ring BA is

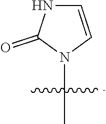 [SMILES: *n1cc[nH]c1=O]

In some embodiments, Ring BA has the structure of formula BA-VI. In some embodiments, X¹′ is —N(−)—, X²′ is —C(O)— and X³′ is —N(R^{B3})—. In some embodiments, X¹′ is —N(−)—, X²′ is —C(O)—, X³′ is —N(R^{B3})—, X⁴′ is —C(R^{B4}′)═, X⁵′ is —N═, X^{6′} is —C(R^{B6}′)═, and X⁷′ is —N═. In some embodiments, X¹′ is —N(−)—, X²′ is —C(O)—, X^{3′} is —N(R^{B3})—, X⁴′ is —C(R^{B4}′)═, X^{5′} is —C(R^{B5}′)═, X^{6′} is —C(R^{B6}′)═, and X⁷ is —C(R^{B7}′)═. In some embodiments, Ring BA is optionally substituted or protected

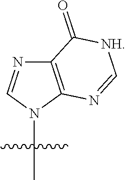 [SMILES: *n1cnc2c(=O)[nH]cnc21]

In some embodiments, Ring BA is

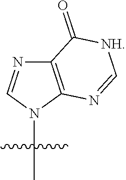 [SMILES: *n1cnc2c(=O)[nH]cnc21]

In some embodiments, Ring BA is optionally substituted or protected

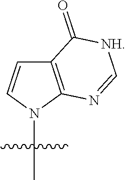 [SMILES: *n1ccc2c(=O)[nH]cnc21]

In some embodiments, Ring BA is

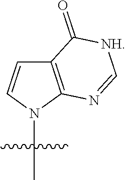 [SMILES: *n1ccc2c(=O)[nH]cnc21]

In some embodiments, Ring BA is optionally substituted or protected

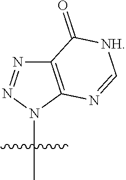 [SMILES: *n1nnc2c(=O)[nH]cnc21]

In some embodiments, Ring BA is

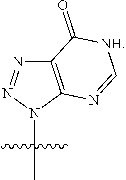 [SMILES: *n1nnc2c(=O)[nH]cnc21]

In some embodiments, Ring BA is optionally substituted or protected

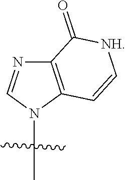 [SMILES: *n1cnc2c(=O)[nH]ccc21]

In some embodiments, Ring BA is

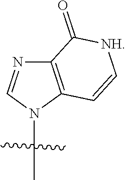 [SMILES: *n1cnc2c(=O)[nH]ccc21]

In some embodiments, Ring BA is optionally substituted or protected

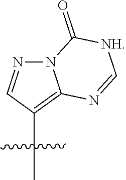 [SMILES: *c1cnn2c(=O)[nH]cnc12]

In some embodiments, Ring BA is

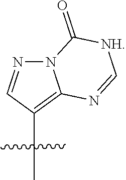 [SMILES: *c1cnn2c(=O)[nH]cnc12]

In some embodiments, Ring BA is optionally substituted or protected

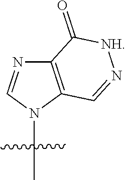 [SMILES: *n1cnc2c(=O)[nH]ncc21]

In some embodiments, Ring BA is

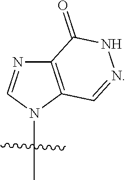 [SMILES: *n1cnc2c(=O)[nH]ncc21]

In some embodiments, Ring BA is optionally substituted or protected

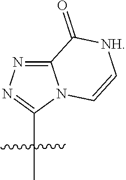 [SMILES: *c1nnc2c(=O)[nH]ccn12]

In some embodiments, Ring BA is

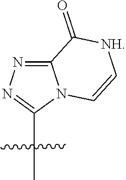 [SMILES: *c1nnc2c(=O)[nH]ccn12]

In some embodiments, Ring BA is optionally substituted or protected

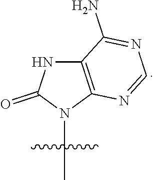 [SMILES: *n1c(=O)[nH]c2c(N)ncnc21]

In some embodiments, Ring BA is

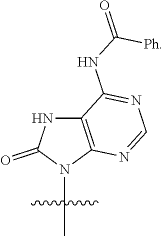 [SMILES: *n1c(=O)[nH]c2c(NC(=O)c3ccccc3)ncnc21]

In some embodiments, Ring BA is

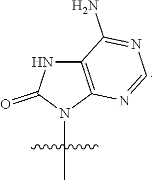 [SMILES: *n1c(=O)[nH]c2c(N)ncnc21]

In some embodiments, Ring BA is optionally substituted or protected

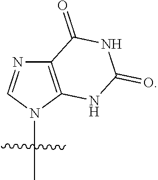 [SMILES: *n1cnc2c(=O)[nH]c(=O)[nH]c21]

In some embodiments, Ring BA is

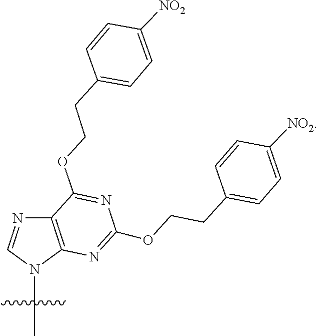 [SMILES: *n1cnc2c(OCCc3ccc([N+](=O)[O-])cc3)nc(OCCc3ccc([N+](=O)[O-])cc3)nc21]

In some embodiments, Ring BA is

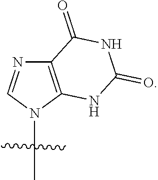 [SMILES: *n1cnc2c(=O)[nH]c(=O)[nH]c21]

In some embodiments, Ring BA is optionally substituted or protected

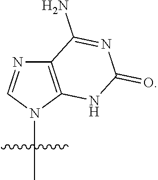 [SMILES: *n1cnc2c(N)nc(=O)[nH]c21]

In some embodiments, Ring BA is

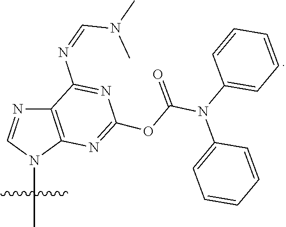 [SMILES: *n1cnc2c(/N=C\N(C)C)nc(OC(=O)N(c3ccccc3)c3ccccc3)nc21]

In some embodiments, Ring BA is

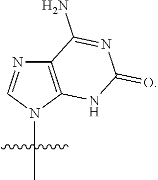 [SMILES: *n1cnc2c(N)nc(=O)[nH]c21]

In some embodiments, Ring BA is optionally substituted or protected

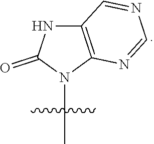 [SMILES: *n1c(=O)[nH]c2cncnc21]

In some embodiments, Ring BA is

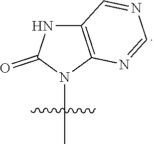 [SMILES: *n1c(=O)[nH]c2cncnc21]

In some embodiments, Ring BA is optionally substituted or protected

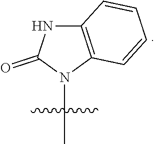 [SMILES: *n1c(=O)[nH]c2ccccc21]

In some embodiments, Ring BA is

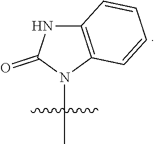 [SMILES: *n1c(=O)[nH]c2ccccc21]

In some embodiments, X¹′ is —N(−)—, X²′ is —C(R^{B2})═, and X³ is —N═. In some embodiments, X¹′ is —N(−)—, X²′ is —C(R^{B2}′)═, X^{3′} is —N═, X⁴ is —C(—N(R^{B4}′)₂)═, X⁵′ is —N═, X⁶′ is —C(O)—, and X⁷′ is —N(R^{B7}′)—. In some embodiments, Ring BA is optionally substituted or protected

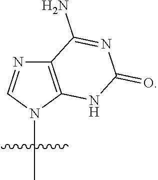 [SMILES: *n1cnc2c(N)nc(=O)[nH]c21]

In some embodiments, Ring BA is

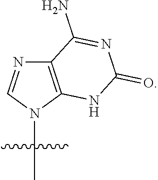 [SMILES: *n1cnc2c(N)nc(=O)[nH]c21]

In some embodiments, X¹ is —C(−)═, X² is —C(O)—, and X³ is —N(R^{B3})—. In some embodiments, X¹ is —C(−)═, X² is —C(O)—, X³ is —N(R^{B3})—, —C(—N(R^{B4})₂)═, and X⁴ is —C(R^{B4})═. In some embodiments, X¹ is —C(−)═, X² is —C(O)—, X³ is —N(R^{B3})—, —C(N(R^{B4})₂)═, X⁴ is —C(R^{B4})═, and X⁶ is —C(R^{B6})═. In some embodiments, each of R^{B3}, R^{B4}, and R^{B6} is independently —H. In some embodiments, Ring BA is optionally substituted or protected

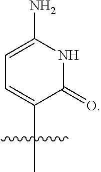 [SMILES: *c1ccc(N)[nH]c1=O]

In some embodiments, Ring BA is

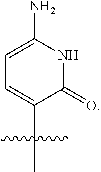 [SMILES: *c1ccc(N)[nH]c1=O]

In some embodiments, Ring BA is optionally substituted or protected

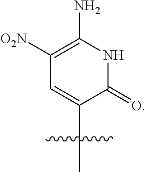 [SMILES: *c1cc([N+](=O)[O-])c(N)[nH]c1=O]

In some embodiments, Ring BA is

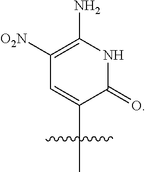 [SMILES: *c1cc([N+](=O)[O-])c(N)[nH]c1=O]

In some embodiments, Ring BA has the structure of

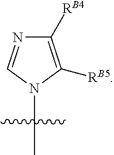

In some embodiments, R^{B4} is optionally substituted aryl. In some embodiments, R^{B4} is optionally substituted

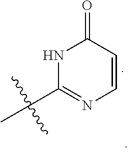 [SMILES: *c1nccc(=O)[nH]1]

In some embodiments, R^{B4} is

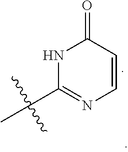 [SMILES: *c1nccc(=O)[nH]1]

In some embodiments, R^{B5} is —H. In some embodiments, R^{B5} is —N(R′)₂. In some embodiments, R^{B5} is —NH₂. In some embodiments, Ring BA is optionally substituted

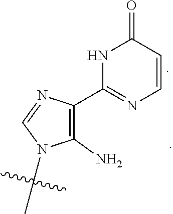 [SMILES: *n1cnc(-c2nccc(=O)[nH]2)c1N]

In some embodiments, Ring BA is

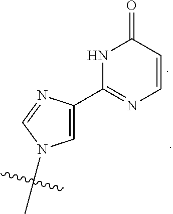 [SMILES: *n1cnc(-c2nccc(=O)[nH]2)c1]

In some embodiments, Ring BA is optionally substituted

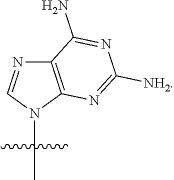 [SMILES: *n1cnc2c(N)nc(N)nc21]

In some embodiments, Ring BA is

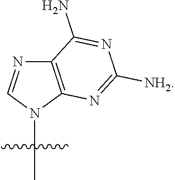 [SMILES: *n1cnc2c(N)nc(N)nc21]

In some embodiments, Ring BA is optionally substituted

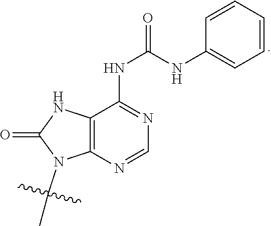 [SMILES: *n1c(=O)[nH]c2c(NC(=O)Nc3ccccc3)ncnc21]

In some embodiments, Ring BA is

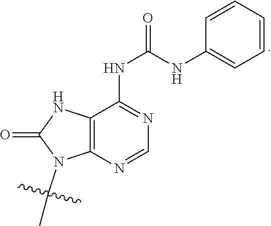 [SMILES: *n1c(=O)[nH]c2c(NC(=O)Nc3ccccc3)ncnc21]

In some embodiments, Ring BA is optionally substituted

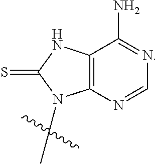 [SMILES: *n1c(=S)[nH]c2c(N)ncnc21]

In some embodiments, Ring BA is

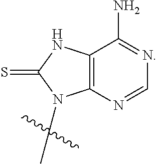 [SMILES: *n1c(=S)[nH]c2c(N)ncnc21]

In some embodiments, Ring BA is optionally substituted

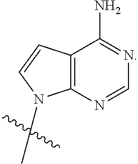 [SMILES: *n1ccc2c(N)ncnc21]

In some embodiments, Ring BA is

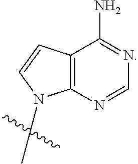 [SMILES: *n1ccc2c(N)ncnc21]

In some embodiments, Ring BA is optionally substituted

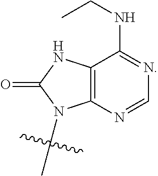 [SMILES: *n1c(=O)[nH]c2c(NCC)ncnc21]

In some embodiments, Ring BA is

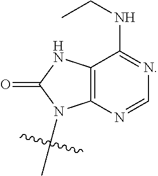 [SMILES: *n1c(=O)[nH]c2c(NCC)ncnc21]

In some embodiments, Ring BA is optionally substituted

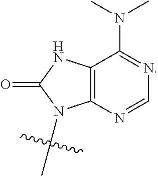 [SMILES: *n1c(=O)[nH]c2c(N(C)C)ncnc21]

In some embodiments, Ring BA

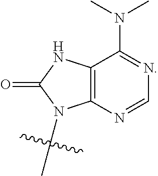 [SMILES: *n1c(=O)[nH]c2c(N(C)C)ncnc21]

In some embodiments, Ring BA is optionally substituted

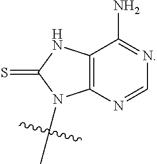 [SMILES: *n1c(=S)[nH]c2c(N)ncnc21]

In some embodiments, Ring BA is

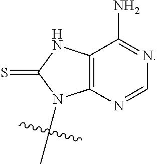 [SMILES: *n1c(=S)[nH]c2c(N)ncnc21]

In some embodiments, Ring BA is optionally substituted

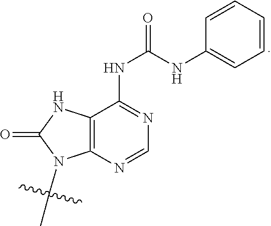 [SMILES: *n1c(=O)[nH]c2c(NC(=O)Nc3ccccc3)ncnc21]

In some embodiments, Ring BA is

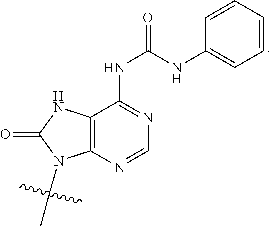 [SMILES: *n1c(=O)[nH]c2c(NC(=O)Nc3ccccc3)ncnc21]

In some embodiments, Ring BA is optionally substituted

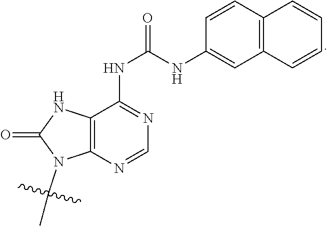 [SMILES: *n1c(=O)[nH]c2c(NC(=O)Nc3ccc4ccccc4c3)ncnc21]

In some embodiments, Ring BA is

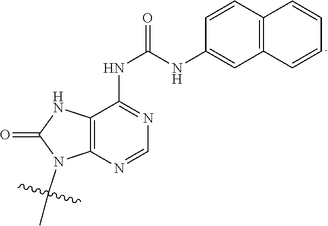 [SMILES: *n1c(=O)[nH]c2c(NC(=O)Nc3ccc4ccccc4c3)ncnc21]

In some embodiments, Ring BA is optionally substituted

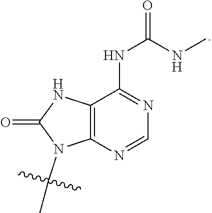 [SMILES: *n1c(=O)[nH]c2c(NC(=O)NC)ncnc21]

In some embodiments, Ring BA is

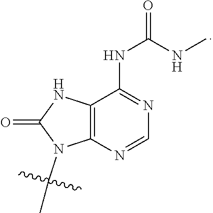 [SMILES: *n1c(=O)[nH]c2c(NC(=O)NC)ncnc21]

In some embodiments, Ring BA is optionally substituted

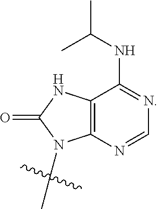 [SMILES: *n1c(=O)[nH]c2c(NC(C)C)ncnc21]

In some embodiments, Ring BA is

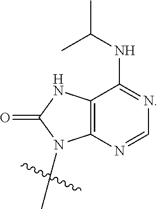 [SMILES: *n1c(=O)[nH]c2c(NC(C)C)ncnc21]

In some embodiments, Ring BA is optionally substituted

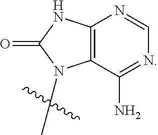 [SMILES: *n1c(=O)[nH]c2ncnc(N)c21]

In some embodiments, Ring BA is

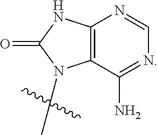 [SMILES: *n1c(=O)[nH]c2ncnc(N)c21]

In some embodiments, Ring BA is optionally substituted

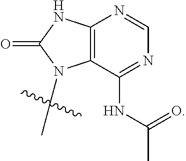 [SMILES: *n1c(=O)[nH]c2ncnc(NC(C)=O)c21]

In some embodiments, Ring BA is

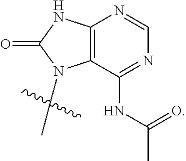 [SMILES: *n1c(=O)[nH]c2ncnc(NC(C)=O)c21]

In some embodiments, Ring BA is optionally substituted

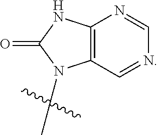 [SMILES: *n1c(=O)[nH]c2ncncc21]

In some embodiments, Ring BA is

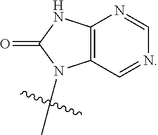 [SMILES: *n1c(=O)[nH]c2ncncc21]

In some embodiments, Ring BA is optionally substituted

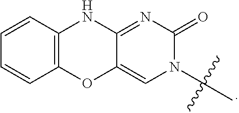 [SMILES: *n1cc2c(nc1=O)Nc1ccccc1O2]

In some embodiments, Ring BA is

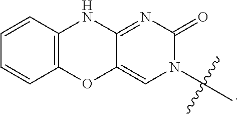 [SMILES: *n1cc2c(nc1=O)Nc1ccccc1O2]

In some embodiments, Ring BA is optionally substituted

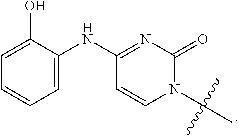 [SMILES: *n1ccc(Nc2ccccc2O)nc1=O]

In some embodiments, Ring BA is

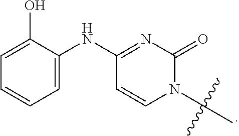 [SMILES: *n1ccc(Nc2ccccc2O)nc1=O]

In some embodiments, Ring BA is optionally substituted

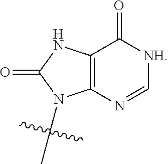 [SMILES: *n1c(=O)[nH]c2c(=O)[nH]cnc21]

In some embodiments, Ring BA is

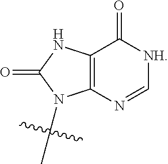 [SMILES: *n1c(=O)[nH]c2c(=O)[nH]cnc21]

In some embodiments, Ring BA is optionally substituted

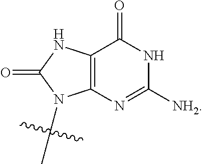 [SMILES: *n1c(=O)[nH]c2c(=O)[nH]c(N)nc21]

In some embodiments, Ring BA is

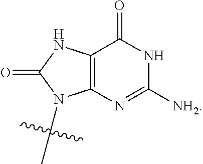 [SMILES: *n1c(=O)[nH]c2c(=O)[nH]c(N)nc21]

As described herein, Ring BA may be optionally substituted. In some embodiments, each of X², X³, X⁴, X⁵, X⁶, X²′, X³′, X⁴′, X⁵′, X⁶′ and X⁷′ is independently and optionally substituted when it is —CH═, —C(OH)═, —C(—NH₂)═, —CH₂—, —C(═NH)—, or —NH—. In some embodiments, each of X², X³, X⁴, X⁵, X⁶, X²′, X^{3′}, X⁴′, X⁵′, X⁶′, and X⁷′ is independently and optionally substituted when it is —CH═, —CH₂—, or —NH—. In some embodiments, each of X², X³, X⁴, X⁵, X⁶, X²′, X³′, X⁴′, X⁵′, X⁶′, and X⁷′ is independently and optionally substituted when it is —CH═. In some embodiments, each of X², X³, X⁴, X⁵, X⁶, X²′, X³′, X⁴′, X⁵′, X⁶′, and X⁷′ is independently and optionally substituted when it is —CH₂—. In some embodiments, each of X², X³, X⁴, X⁵, X⁶, X²′, X³′, X⁴′, X⁵′, X⁶′, and X⁷′ is independently and optionally substituted when it is —NH—. In some embodiments, X² is optionally substituted —CH═, —C(OH)═, —C(—NH₂)═, —CH₂—, —C(═NH)—, or —NH—. In some embodiments, X³ is optionally substituted —CH═, —C(OH)═, —C(—NH₂)═, —CH₂—, —C(═NH)—, or —NH—. In some embodiments, X⁴ is optionally substituted —CH═, —C(OH)═, —C(—NH₂)═, —CH₂—, —C(═NH)—, or —NH—. In some embodiments, X⁵ is optionally substituted —CH═, —C(OH)═, —C(—NH₂)═, —CH₂—, —C(═NH)—, or —NH—. In some embodiments, X⁶ is optionally substituted —CH═, —C(OH)═, —C(—NH₂)═, —CH₂—, —C(═NH)—, or —NH—. In some embodiments, X²′ is optionally substituted —CH═, —C(OH)═, —C(—NH₂)═, —CH₂—, —C(═NH)—, or —NH—. In some embodiments, X³′ is optionally substituted —CH═, —C(OH)═, —C(—NH₂)═, —CH₂—, —C(═NH)—, or —NH—. In some embodiments, X⁴′ is optionally substituted —CH═, —C(OH)═, —C(—NH₂)═, —CH₂—, —C(═N)—, or —NH—. In some embodiments, X⁵′ is optionally substituted —CH═, —C(OH)═, —C(—NH₂)═, —CH₂—, —C(═NH)—, or —NH—. In some embodiments, X⁶′ is optionally substituted —CH═, —C(OH)═, —C(—NH₂)═, —CH₂—, —C(═NH)—, or —NH—. In some embodiments, XT is optionally substituted —CH═, —C(OH)═, —C(—NH₂)═, —CH₂—, —C(═NH)—, or —NH—.

In some embodiments, X⁸′ is C. In some embodiments, X⁸′ is N. In some embodiments, X⁹′ is C. In some embodiments, X⁹′ is N. In some embodiments, X⁸′ is C and X⁹′ is C. In some embodiments, X⁸′ is C and X⁹′ is N. In some embodiments, X⁸′ is N and X^{9′} is C. In some embodiments, X⁸′ is N and X⁹′ is N.

In some embodiments, Ring BA is aromatic. In some embodiments, Ring BA is aromatic and has one or more, e.g., 1-5, nitrogen ring atoms. In some embodiments, Ring BA comprises an optionally substituted aromatic ring that has one or more, e.g., 1-5, nitrogen ring atoms.

In some embodiments provided oligonucleotides comprising certain nucleobases (e.g., b001A, b002A, b008U, C, A, etc.) opposite to target adenosines can among other things provide improved editing efficiency (e.g., compared to a reference nucleobase such as U). In some embodiments, an opposite nucleoside is linked to an I to its 3′ side.

In some embodiments, a nucleobase is Ring BA as described herein. In some embodiments, an oligonucleotide comprises one or more Ring BA as described herein.

In some embodiments, an opposite nucleoside is abasic, e.g., having the structure of

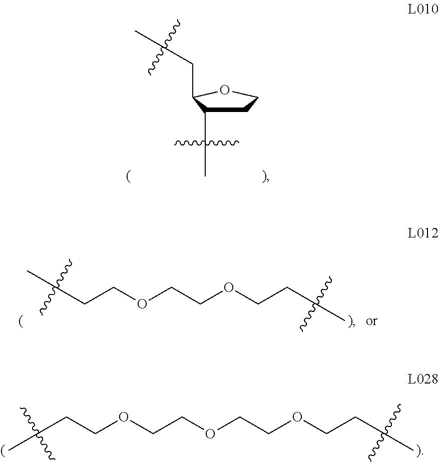

Abasic nucleosides may also be utilized in other portions of oligonucleotides, and oligonucleotides may comprise one or more (e.g., 1, 2, 3, 4, 5, or more), optionally consecutive, abasic nucleosides. In some embodiments, a first domain comprises one or more optionally consecutive, abasic nucleosides. In some embodiments, an oligonucleotide comprises one and no more than one abasic nucleoside. In some embodiments, each abasic nucleoside is independently in a first domain or a first subdomain of a second domain. In some embodiments, each abasic nucleoside is independently in a first domain. In some embodiments, each abasic nucleoside is independently in a first subdomain of a second domain. In some embodiments, an abasic nucleoside is opposite to a target adenosine. In some embodiments, a single abasic nucleoside may replace one or more nucleosides each of which independently comprises a nucleobase in a reference oligonucleotide, for example, L010 may be utilized to replace 1 nucleoside which comprises a nucleobase, L012 may be utilized to replace 1, 2 or 3 nucleosides each of which independently comprises a nucleobase, and L028 may be utilized to replace 1, 2 or 3 nucleosides each of which independently comprises a nucleobase. In some embodiments, a basic nucleoside is linked to its 3′ immediate nucleoside (which is optionally abasic) through a stereorandom linkage (e.g., a stereorandom phosphorothioate internucleotidic linkage). In some embodiments, each basic nucleoside is independently linked to its 3′ immediate nucleoside (which is optionally abasic) through a stereorandom linkage (e.g., a stereorandom phosphorothioate internucleotidic linkage).

In some embodiments, a modified nucleobase opposite to a target adenine can greatly improve properties and/or activities of an oligonucleotide. In some embodiments, a modified nucleobase at the opposite position can provide high activities even when there is a G next to it (e.g., at the 3′ side), and/or other nucleobases, e.g. C, provide much lower activities or virtually no detect activates.

In some embodiments, a second domain comprises one or more sugars comprising two 2′-H (e.g., natural DNA sugars). In some embodiments, a second domain comprises one or more sugars comprising 2′-OH (e.g., natural RNA sugars). In some embodiments, a second domain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17. 18, 19, or 20, etc.) modified sugars. In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a 2′-OR modified sugar, wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl.

In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a second domain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a second domain are independently modified sugars with a modification that is not 2′-F. In some embodiments, modified sugars of a second domain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, a second domain comprises one or more 2′-F modified sugars. In some embodiments, a second domain comprises no 2′-F modified sugars. In some embodiments, a second domain comprises one or more bicyclic sugars and/or 2′-OR modified sugars wherein R is not —H. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second domain comprises 2′-F. In some embodiments, no more than about 50% of sugars in a second domain comprises 2′-F. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second domain comprises 2′-MOE. In some embodiments, no more than about 50% of sugars in a second domain comprises 2′-MOE. In some embodiments, no sugars in a second domain comprises 2′-MOE.

In some embodiments, a second domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%0, % 40%, 50%, 60%, 65%, 70%, 75%, 80%, 85%, 90%, 95%, or 100%, etc.) of internucleotidic linkages in a second domain are modified internucleotidic linkages. In some embodiments, each internucleotidic linkage in a second domain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a second domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second domain is chirally controlled. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a second domain is Sp. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a second domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second domain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two second domain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a second domain is bonded to two nucleosides of the second domain. In some embodiments, an internucleotidic linkage bonded to a nucleoside in a first domain and a nucleoside in a second domain may be properly considered an internucleotidic linkage of a second domain. In some embodiments, a high percentage (e.g., relative to Rp internucleotidic linkages and/or natural phosphate linkages) of Sp internucleotidic linkages provide improved properties and/or activities, e.g., high stability and/or high adenosine editing activity.

In some embodiments, a second domain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90% 95%, or 100%, etc. of all internucleotidic linkages in a second domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a second domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a second domain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a second domain is independently chirally controlled. In some embodiments, each is independently Sp or Rp. In some embodiments, a high level is Sp as described herein. In some embodiments, each phosphorothioate internucleotidic linkage in a second domain is chirally controlled and is Sp. In some embodiments, one or more, e.g., about 1-5 (e.g., about 1, 2, 3, 4, or 5) is Rp.

In some embodiments, each phosphorothioate internucleotidic linkage in a second domain is independently chirally controlled. In some embodiments, each is independently Sp or Rp. In some embodiments, a high level is Sp as described herein. In some embodiments, each phosphorothioate internucleotidic linkage in a second domain is chirally controlled and is Sp. In some embodiments, one or more, e.g., about 1-5 (e.g., about 1, 2, 3, 4, or 5) is Rp.

In some embodiments, as illustrated in certain examples, a second domain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a second domain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a second domain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a second domain. In some embodiments, the last two or three or four internucleotidic linkages of a second domain comprise at least one internucleotidic linkage that is not a non-negatively charged internucleotidic linkage. In some embodiments, the last two or three or four internucleotidic linkages of a second domain comprise at least one internucleotidic linkage that is not n001.

In some embodiments, the internucleotidic linkage linking the last two nucleosides of a second domain is a non-negatively charged internucleotidic linkage. In some embodiments, the internucleotidic linkage linking the last two nucleosides of a second domain is a Sp non-negatively charged internucleotidic linkage. In some embodiments, the internucleotidic linkage linking the last two nucleosides of a second domain is a Rp non-negatively charged internucleotidic linkage. In some embodiments, the internucleotidic linkage linking the last two nucleosides of a second domain is a phosphorothioate internucleotidic linkage. In some embodiments, the internucleotidic linkage linking the last two nucleosides of a second domain is a Sp phosphorothioate internucleotidic linkage. In some embodiments, the last two nucleosides of a second domain are the last two nucleosides of an oligonucleotide. In some embodiments, the internucleotidic linkage linking the first two nucleosides of a second domain is a non-negatively charged internucleotidic linkage. In some embodiments, the internucleotidic linkage linking the first two nucleosides of a second domain is a Sp non-negatively charged internucleotidic linkage. In some embodiments, the internucleotidic linkage linking the first two nucleosides of a second domain is a Rp non-negatively charged internucleotidic linkage. In some embodiments, the internucleotidic linkage linking the first two nucleosides of a second domain is a phosphorothioate internucleotidic linkage. In some embodiments, the internucleotidic linkage linking the first two nucleosides of a second domain is a Sp phosphorothioate internucleotidic linkage. In some embodiments, a non-negatively charged internucleotidic linkage is a neutral internucleotidic linkage such as n001.

In some embodiments, one or more (e.g., about or at least about 1, 2, 3, 4, 5, 6, 7, 8, 9, 10, 11, 12, 13, 14, 15, 16, 17, 18, 19, or 20, or about or at least about 10%, 20%, 25%, 30%, 35%, 40%, 50%, 60%, 70%, 75%, 80%, 85%, 90% or 95% of all internucleotidic linkage in a second domain) chiral internucleotidic linkage in a second domain are not chirally controlled. In some embodiments, all chiral internucleotidic linkages in a second domain are not chirally controlled. In some embodiments, a second domain comprises one or more blocks of chirally controlled internucleotidic linkages and one or more blocks of non-chirally controlled internucleotidic linkages. In some embodiments, in each block of chirally controlled internucleotidic linkages there are about 1-20, e.g., about 1, 2, 3, 4, 5, 6, 7, 8, 9, 10, 11, 12, 13, 14, 15, 16, 17, 18, 19 or 20 chiral internucleotidic linkages, and each is independently chirally controlled. In some embodiments, in each block ofnon-chirally controlled internucleotidic linkages there are about 1-20, e.g., about 1, 2, 3, 4, 5, 6, 7, 8, 9, 10, 11, 12, 13, 14, 15, 16, 17, 18, 19 or 20 chiral internucleotidic linkages, and each is independently non-chirally controlled.

In some embodiments, a second domain comprises one or more natural phosphate linkages. In some embodiments, a second domain contains no natural phosphate linkages.

In some embodiments, a first domain is to the 5′-side of a second domain. In some embodiments, a first domain is to the 3′-side of a second domain.

In some embodiments, a second domain recruits, promotes or contribute to recruitment of, a protein such as an ADAR protein. In some embodiments, a second domain recruits, or promotes or contribute to interactions with, a protein such as an ADAR protein. In some embodiments, a second domain contacts with a RNA binding domain (RBD) of ADAR. In some embodiments, a second domain contacts with a catalytic domain of ADAR which has a deaminase activity. In some embodiments, various nucleobases, sugars and/or internucleotidic linkages may interact with one or more residues of proteins, e.g., ADAR proteins. Without the intention to be limited by any theory, in some embodiments, a first portion and a second portion, though separated by a gap in nucleobase sequence, may be in spatial proximity, e.g., in folded or secondary structures of target nucleic acids such as mRNA; in some embodiments, provided oligonucleotides, with domains to form duplexes with two portions of target nucleic acids, enable, promote, encourage or stabilize certain spatial structures of target nucleic acids such as mRNA.

In some embodiments, a second domain comprises or consists of a first subdomain as described herein. In some embodiments, a second domain comprises or consists of a second subdomain as described herein. In some embodiments, a second domain comprises or consists of a third subdomain as described herein. In some embodiments, a second domain comprises or consists of a first subdomain, a second subdomain and a third subdomain from 5′ to 3′. Certain embodiments of such subdomains are described below. First Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a first subdomain are described below as examples. In some embodiments, a first subdomain comprise a nucleoside opposite to target adenosine to be modified (e.g., conversion to I).

In some embodiments, a first subdomain has a length of about 1-50, 1-40, 1-30, 1-20 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 1, 2, 3, 4, 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a first subdomain has a length of about 5-30 nucleobases. In some embodiments, a first subdomain has a length of about 10-30 nucleobases. In some embodiments, a first subdomain has a length of about 10-20 nucleobases. In some embodiments, a first subdomain has a length of about 5-15 nucleobases. In some embodiments, a first subdomain has a length of about 13-16 nucleobases. In some embodiments, a first subdomain has a length of about 6-12 nucleobases. In some embodiments, a first subdomain has a length of about 6-9 nucleobases. In some embodiments, a first subdomain has a length of about 1-10 nucleobases. In some embodiments, a first subdomain has a length of about 1-7 nucleobases. In some embodiments, a first subdomain has a length of about 1-5 nucleobases. In some embodiments, a first subdomain has a length of about 1-3 nucleobases. In some embodiments, a first subdomain has a length of 1 nucleobase. In some embodiments, a first subdomain has a length of 2 nucleobases. In some embodiments, a first subdomain has a length of 3 nucleobases. In some embodiments, a first subdomain has a length of 4 nucleobases. In some embodiments, a first subdomain has a length of 5 nucleobases. In some embodiments, a first subdomain has a length of 6 nucleobases. In some embodiments, a first subdomain has a length of 7 nucleobases. In some embodiments, a first subdomain has a length of 8 nucleobases. In some embodiments, a first subdomain has a length of 9 nucleobases. In some embodiments, a first subdomain has a length of 10 nucleobases. In some embodiments, a first subdomain has a length of 11 nucleobases. In some embodiments, a first subdomain has a length of 12 nucleobases. In some embodiments, a first subdomain has a length of 13 nucleobases. In some embodiments, a first subdomain has a length of 14 nucleobases. In some embodiments, a first subdomain has a length of 15 nucleobases.

In some embodiments, a first subdomain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of a second domain. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a first subdomain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a first subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a first subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a first subdomain is fully complementary to a target nucleic acid.

In some embodiments, a first subdomain comprises one or more modified nucleobases.

In some embodiments, a first subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA.

In some embodiments, a first subdomain comprises one or more sugars comprising two 2′-H (e.g., natural DNA sugars). In some embodiments, a first subdomain comprises one or more sugars comprising 2′—OH (e.g., natural RNA sugars). In some embodiments, a first subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first subdomain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%. 75%-90%, 75%-95%, 75%-100%, 80%-85%, 80%-90%, 80%-95%, 80%-100%, 85%-90%, 85%-95%, 85%-100%, 90%-95%, 90%-100%1%, 1, 20%, 30%, 40%, 50%, 60%, 65%, 70%, 75%, 80%, 85%, 90%, 95%, or 100%, etc.) of all sugars in a first subdomain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%,90%-100%,10%, 20%, 30%, 40%, 50%, 60%, 65%, 70%, 75%, 80%, 85%, 90%, 95%, or 100%, etc.) of all sugars in a first subdomain are independently a 2′-OR modified sugar. wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl.

In some embodiments, a first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%,90%-100%,10%, 20%, 30%, 40%, 50%, 60%, 65%, 70%, 75%, 80%, 85%, 90%, 95%, or 100%, etc.) of sugars in a first subdomain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a first subdomain are independently modified sugars with a modification that is not 2′-F. In some embodiments, modified sugars of a first subdomain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, each sugar in a first domain is a 2′-F modified sugar.

In some embodiments, a first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)2 modification, wherein each R is independently optionally substituted C₁₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a first subdomain are independently modified sugars selected from a bicyclic sugar (e.g., a LNA sugar), an acyclic sugar (e.g., a UNA sugar), a sugar with a 2′-OR modification, or a sugar with a 2′-N(R)2 modification, wherein each R is independently optionally substituted C₁₋₆ aliphatic. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, each sugar in a first subdomain independently comprises a 2′-OR modification, wherein R is optionally substituted C₁₋₆ aliphatic, or a 2′-O-L^{B}-4′ modification. In some embodiments, each sugar in a first subdomain independently comprises a 2′-OR modification, wherein R is optionally substituted C₁₋₆ aliphatic, or a 2′-O-L^{B}-4′ modification, wherein L^{B} is optionally substituted —CH₂—. In some embodiments, each sugar in a first subdomain independently comprises 2′-OMe.

In some embodiments, a first subdomain comprises one or more 2′-F modified sugars. In some embodiments, a first subdomain comprises no 2′-F modified sugars. In some embodiments, a first subdomain comprises one or more bicyclic sugars and/or 2′-OR modified sugars wherein R is not —H. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) 2′-OMe modified sugars. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) consecutive 2′-OMe modified sugars. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first subdomain comprises 2′-F. In some embodiments, no more than about 50% of sugars in a first subdomain comprises 2′-F. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first subdomain comprises 2′-MOE. In some embodiments, no more than about 50% of sugars in a first subdomain comprises 2′-MOE. In some embodiments, no sugars in a first subdomain comprises 2′-MOE.

In some embodiments, a first subdomain contains more 2′-OR modified sugars than 2′-F modified sugars. In some embodiments, each sugar in a first subdomain is independently a 2′-OR modified sugar or a 2′-F modified sugar. In some embodiments, a first subdomain contains only 3 nucleosides, two of which are independently 2′-OR modified sugars and one is a 2′-F modified sugar. In some embodiments, the 2′-F modified nucleoside is at the 3′-end of the first subdomain and connects to a second subdomain. In some embodiments, each 2′-OR modified sugar is independently a 2′-OR modified sugar wherein R is optionally substituted C₁₋₆ aliphatic, or a 2′-O-L^{B}-4′ modification. In some embodiments, each 2′-OR modified sugar is independently 2′-OR modified sugar wherein R is optionally substituted C₁₋₆ aliphatic. In some embodiments, each 2′-OR modified sugar is independently a 2′-OMe or 2′-MOE modified sugar. In some embodiments, each 2′-OR modified sugar is independently a 2′-OMe modified sugar. In some embodiments, each 2′-OR modified sugar is independently a 2′-MOE modified sugar. In some embodiments, a sugar is 2′-OMe modified and a sugar is 2′-MOE. In some embodiments, a first subdoman contains only 3 nucleosides which are N₂, N₃ and N₄.

In some embodiments, a first subdomain comprise about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a first subdomain are modified internucleotidic linkages. In some embodiments, each internucleotidic linkage in a first subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first subdomain is chirally controlled. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first subdomain is Sp. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a first subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first subdomain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two first subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a first subdomain is bonded to two nucleosides of the first subdomain. In some embodiments, an internucleotidic linkage bonded to a nucleoside in a first subdomain and a nucleoside in a second subdomain may be properly considered an internucleotidic linkage of a first subdomain. In some embodiments, an internucleotidic linkage bonded to a nucleoside in a first subdomain and a nucleoside in a second subdomain is a modified internucleotidic linkage; in some embodiments, it is a chiral internucleotidic linkage; in some embodiments, it is chirally controlled; in some embodiments, it is Rp; in some embodiments, it is Sp.

In some embodiments, a first subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a first subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a first subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 5% 70%, 75%, 80%, 85%, 90%, 95%, or 100%, etc. of all chirally controlled internucleotidic linkages in a first subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a first subdomain is independently chirally controlled. In some embodiments, each is independently Sp or Rp. In some embodiments, a high level is Sp as described herein. In some embodiments, each phosphorothioate internucleotidic linkage in a first subdomain is chirally controlled and is Sp. In some embodiments, one or more, e.g., about 1-5 (e.g., about 1, 2, 3, 4, or 5) is Rp.

In some embodiments, as illustrated in certain examples, a first subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a first subdomain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a first subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a first subdomain. In some embodiments, the last two or three or four internucleotidic linkages of a first subdomain comprise at least one internucleotidic linkage that is not a non-negatively charged internucleotidic linkage. In some embodiments, the last two or three or four internucleotidic linkages of a first subdomain comprise at least one internucleotidic linkage that is not n001. In some embodiments, the internucleotidic linkage linking the last two nucleosides of a first subdomain is a non-negatively charged internucleotidic linkage. In some embodiments, the internucleotidic linkage linking the last two nucleosides of a first subdomain is a Sp non-negatively charged internucleotidic linkage. In some embodiments, the internucleotidic linkage linking the last two nucleosides of a first subdomain is a Rp non-negatively charged internucleotidic linkage. In some embodiments, the internucleotidic linkage linking the last two nucleosides of a first subdomain is a phosphorothioate internucleotidic linkage. In some embodiments, the internucleotidic linkage linking the last two nucleosides of a first subdomain is a Sp phosphorothioate internucleotidic linkage. In some embodiments, the internucleotidic linkage linking the first two nucleosides of a first subdomain is a non-negatively charged internucleotidic linkage. In some embodiments, the internucleotidic linkage linking the first two nucleosides of a first subdomain is a Sp non-negatively charged internucleotidic linkage. In some embodiments, the internucleotidic linkage linking the first two nucleosides of a first subdomain is a Rp non-negatively charged internucleotidic linkage. In some embodiments, the internucleotidic linkage linking the first two nucleosides of a first subdomain is a phosphorothioate internucleotidic linkage. In some embodiments, the internucleotidic linkage linking the first two nucleosides of a first subdomain is a Sp phosphorothioate internucleotidic linkage. In some embodiments, a non-negatively charged internucleotidic linkage is a neutral internucleotidic linkage such as n001.

In some embodiments, a first subdomain comprises one or more natural phosphate linkages. In some embodiments, a first subdomain contains no natural phosphate linkages. In some embodiments, one or more 2′-OR modified sugars wherein R is not —H are independently bonded to a natural phosphate linkage. In some embodiments, one or more 2′-OR modified sugars wherein R is optionally substituted C₁₋₆ aliphatic are independently bonded to a natural phosphate linkage. In some embodiments, one or more 2′-OMe modified sugars are independently bonded to a natural phosphate linkage. In some embodiments, one or more 2′-MOE modified sugars are independently bonded to a natural phosphate linkage. In some embodiments, each 2′-MOE modified sugar is independently bonded to a natural phosphate linkage. In some embodiments, 50% or more (e.g., 50%-100%, 50%-90%, 50-80%, or about 50%, 60%, 66%, 70%, 75%, 80%, 90% or more) 2′-OR modified sugars wherein R is not —H are independently bonded to a natural phosphate linkage. In some embodiments, 50% or more (e.g., 50%-100%, 50%-90%, 50-80%, or about 50%, 60%, 66%, 70%, 75%, 80%, 90% or more) 2′-OMe modified sugars are independently bonded to a natural phosphate linkage. In some embodiments, 50% or more (e.g., 50%-100%,50%-90%, 50-80%, or about 50%, 60%, 66%, 70%, 75%, 80%, 90% or more) 2′-MOE modified sugars are independently bonded to a natural phosphate linkage. In some embodiments, 50% or more (e.g., 50%-100%, 50%-90%, 50-80%, or about 50%, 60%, 66%, 70%, 75%, 80%, 90% or more) internucleotidic linkages bonded to two 2′-OR modified sugars are independently natural phosphate linkages. In some embodiments, 50% or more (e.g., 50%-100%, 50%-90%, 50-80%, or about 50%, 60%, 66%, 70%, 75%, 80%, 90% or more) internucleotidic linkages bonded to two 2′-OMe or 2′-MOE modified sugars are independently natural phosphate linkages.

In some embodiments, a first subdomain comprises a 5′-end portion, e.g., one having a length of about 1-20, 1-15, 1-10, 3-8, or about 1, 2, 3, 4, 5, 6, 7, 8, 9, or 10 nucleobases. In some embodiments, a 5′-end portion has a length of about 3-6 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 5′-end portion comprises the 5′-end nucleobase of a first subdomain.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, one or more (e.g., about 1-10, or about 1, 2, 3, 4, 5, 6, 7, 8, 9, or 10) internucleotidic linkages of a 5′-end portion are independently a modified internucleotidic linkage. In some embodiments, one or more (e.g., about 1-10, or about 1, 2, 3, 4, 5, 6, 7, 8, 9, or 10) internucleotidic linkages of a 5′-end portion are independently a chiral internucleotidic linkage. In some embodiments, one or more (e.g., about 1-10, or about 1, 2, 3, 4, 5, 6, 7, 8, 9, or 10) internucleotidic linkages ofa 5′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 5′-end portion are Rp. In some embodiments, one or more (e.g., about 1-10, or about 1, 2, 3, 4, 5, 6, 7, 8, 9, or 10) internucleotidic linkages of a 5′-end portion are Sp. In some embodiments, each internucleotidic linkage of a 5′-end portion is Sp.

In some embodiments, a 5′-end portion comprises one or more (e.g., about 1-10, or about 1, 2, 3, 4, 5, 6, 7, 8, 9, or 10) mismatches as described herein. In some embodiments, a 5′-end portion comprises one or more (e.g., about 1-10, or about 1, 2, 3, 4, 5, 6, 7, 8, 9, or 10) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a first subdomain comprises a 3′-end portion, e.g., one having a length of about 1-20, 1-15, 1-10, 1-5, 1-3, 3-8, or about 1, 2, 3, 4, 5, 6, 7, 8, 9, or 10 nucleobases. In some embodiments, a 3′-end portion has a length of about 1-3 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 3′-end portion comprises the 3′-end nucleobase of a first subdomain. In some embodiments, a first subdomain comprises or consists of a 5′-end portion and a 3′-end portion.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 3′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, compared to a 5′-end portion, a 3′-end portion contains a higher level (in numbers and/or percentage) of 2′-F modified sugars and/or sugars comprising two 2′-H (e.g., natural DNA sugars), and/or a lower level (in numbers and/or percentage) of other types of modified sugars, e.g., bicyclic sugars and/or sugars with 2′-OR modifications wherein R is independently optionally substituted C₁₋₆ aliphatic. In some embodiments, compared to a 5′-end portion, a 3′-end portion contains a higher level of 2′-F modified sugars and/or a lower level of 2′-OR modified sugars wherein R is optionally substituted C₁₋₆ aliphatic. In some embodiments, compared to a 5′-end portion, a 3′-end portion contains a higher level of 2′-F modified sugars and/or a lower level of 2′-OMe modified sugars. In some embodiments, compared to a 5′-end portion, a 3′-end portion contains a lower level of 2′-F modified sugars and/or a higher level of 2′-OR modified sugars wherein R is optionally substituted C₁₋₆ aliphatic. In some embodiments, compared to a 5′-end portion, a 3′-end portion contains a lower level of 2′-F modified sugars and/or a higher level of 2′-OMe modified sugars. In some embodiments, compared to a 5′-end portion, a 3′-end portion contains a higher level of natural DNA sugars and/or a lower level of 2′-OR modified sugars wherein R is optionally substituted C₁₋₆ aliphatic. In some embodiments, compared to a 5′-end portion, a 3′-end portion contains a higher level of natural DNA sugars and/or a lower level of 2′-OMe modified sugars. In some embodiments, a 3′-end portion contains low levels (e.g., no more than 50%, 40%, 30%, 25%, 20%, or 10%, or no more than 1, 2, 3, 4, 5, 6, 7, 8, 9, or 10) of modified sugars which are bicyclic sugars or sugars comprising 2′-OR wherein R is optionally substituted C₁i6 aliphatic (e.g., methyl). In some embodiments, a 3′-end portion contains no modified sugars which are bicyclic sugars or sugars comprising 2′-OR wherein R is optionally substituted C₁₋₆ aliphatic (e.g., methyl).

In some embodiments, one or more modified sugars independently comprise 2′-F. In some embodiments, no modified sugars comprises 2′-OMe or other 2′-OR modifications wherein R is optionally substituted C₁₋₆ aliphatic. In some embodiments, each sugar of a 3′-end portion independently comprises two 2′-H or a 2′-F modification. In some embodiments, a 3′-end portion comprises 1, 2, 3, 4, or 5 2′-F modified sugars. In some embodiments, a 3′-end portion comprises 1-3 2′-F modified sugars. In some embodiments, a 3′-end portion comprises 1, 2, 3, 4, or 5 natural DNA sugars. In some embodiments, a 3′-end portion comprises 1-3 natural DNA sugars.

In some embodiments, one or more (e.g., about 1-10, or about 1, 2, 3, 4, 5, 6, 7, 8, 9, or 10) internucleotidic linkages of a 3′-end portion are independently a modified internucleotidic linkage. In some embodiments, one or more (e.g., about 1-10, or about 1, 2, 3, 4, 5, 6, 7, 8, 9, or 10) internucleotidic linkages of a 3′-end portion are independently a chiral internucleotidic linkage. In some embodiments, one or more (e.g., about 1-10, or about 1, 2, 3, 4, 5, 6, 7, 8, 9, or 10) internucleotidic linkages ofa 3′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 3′-end portion are Rp. In some embodiments, one or more (e.g., about 1-10, or about 1, 2, 3, 4, 5, 6, 7, 8, 9, or 10) internucleotidic linkages of a 3′-end portion are Sp. In some embodiments, each internucleotidic linkage of a 3′-end portion is Sp. In some embodiments, a 3′-end portion contains a higher level (in number and/or percentage) of Rp internucleotidic linkage and/or natural phosphate linkage compared to a 5′-end portion.

In some embodiments, a 3′-end portion comprises one or more (e.g., about 1-10, or about 1, 2, 3, 4, 5, 6, 7, 8, 9, or 10) mismatches as described herein. In some embodiments, a 3′-end portion comprises one or more (e.g., about 1-10, or about 1, 2, 3, 4, 5, 6, 7, 8, 9, or 10) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a first subdomain recruits, promotes or contribute to recruitment of, a protein such as an ADAR protein, e.g., ADAR1, ADAR2, etc. In some embodiments, a first subdomain recruits, or promotes or contribute to interactions with, a protein such as an ADAR protein. In some embodiments, a first subdomain contacts with a RNA binding domain (RBD) of ADAR. In some embodiments, a first subdomain contacts with a catalytic domain of ADAR which has a deaminase activity. In some embodiments, a first subdomain contact with a domain that has a deaminase activity of ADAR1. In some embodiments, a first subdomain contact with a domain that has a deaminase activity of ADAR2. In some embodiments, various nucleobases, sugars and/or internucleotidic linkages of a first subdomain may interact with one or more residues of proteins, e.g., ADAR proteins.

### Second Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a second subdomain are described below as examples. In some embodiments, a second subdomain comprise a nucleoside opposite to a target adenosine to be modified (e.g., conversion to I). In some embodiments, a second subdomain comprises one and no more than one nucleoside opposite to a target adenosine. In some embodiments, each nucleoside opposite to a target adenosine of an oligonucleotide is in a second subdomain.

In some embodiments, a second subdomain has a length of about 1-10, 1-5, 1-3, or about 1, 2, 3, 4, 5, 6, 7, 8, 9, or 10 nucleobases. In some embodiments, a second subdomain has a length of about 1-10 nucleobases. In some embodiments, a second subdomain has a length of about 1-5 nucleobases. In some embodiments, a second subdomain has a length of about 1-3 nucleobases. In some embodiments, a second subdomain has a length of 1 nucleobase. In some embodiments, a second subdomain has a length of 2 nucleobases. In some embodiments, a second subdomain has a length of 3 nucleobases. In some embodiments, all the nucleosides in a second subdomain are 5′-N₁N0N₋₁-3′.

In some embodiments, one or more (e.g., 1-20, 1, 2, 3, 4, 5, 6, 7, 8, 9, or 10, etc.) mismatches exist in a second subdomain when an oligonucleotide is aligned with a target nucleic acid for complementarity. In some embodiments, there is I mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, a second subdomain comprises one and no more than one mismatch. In some embodiments, a second subdomain comprises two and no more than two mismatches. In some embodiments, a second subdomain comprises two and no more than two mismatches, wherein one mismatch is between a target adenosine and its opposite nucleoside, and/or one mismatch is between a nucleoside next to a target adenosine and its corresponding nucleoside in an oligonucleotide. In some embodiments, a mismatch between a nucleoside next to a target adenosine and its corresponding nucleoside in an oligonucleotide is a wobble. In some embodiments, a wobble is I-C. In some embodiments, C is next to a target adenosine, e.g., immediately to its 3′ side.

In some embodiments, one or more (e.g., 1-20, 1, 2, 3, 4, 5, 6, 7, 8, 9, or 10, etc.) wobbles exist in a second subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a second subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a second subdomain is fully complementary to a target nucleic acid.

In some embodiments, a second subdomain comprises one or more modified nucleobases.

In some embodiments, a second subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA. For example, in some embodiments, an opposite nucleobase is selected from

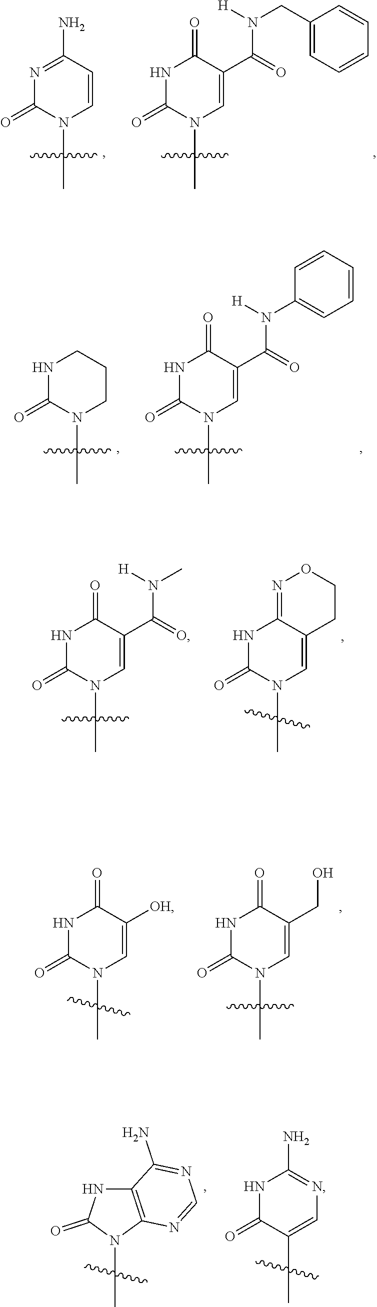

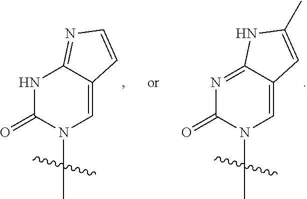 [SMILES: *n1cc2ccnc-2[nH]c1=O ; *n1cc2cc(C)[nH]c2nc1=O]

In some embodiments, an opposite nucleobase is

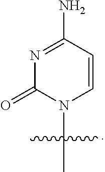 [SMILES: *n1ccc(N)nc1=O]

In some embodiments, an opposite nucleobase is

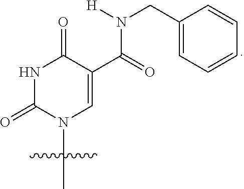 [SMILES: *n1cc(C(=O)NCc2ccccc2)c(=O)[nH]c1=O]

In some embodiments, an opposite nucleobase is

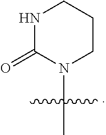 [SMILES: *N1CCCNC1=O]

In some embodiments, an opposite nucleobase is

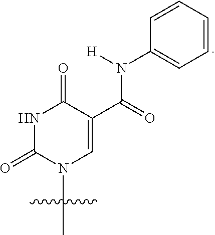 [SMILES: *n1cc(C(=O)Nc2ccccc2)c(=O)[nH]c1=O]

In some embodiments, an opposite nucleobase is

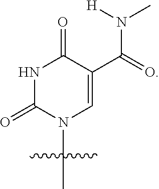 [SMILES: *n1cc(C(=O)NC)c(=O)[nH]c1=O]

In some embodiments, an opposite nucleobase is

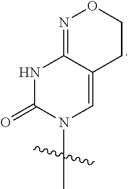 [SMILES: *N1C=C2CCON=C2NC1=O]

In some embodiments, an opposite nucleobase is

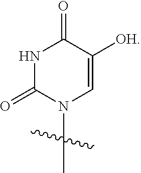 [SMILES: *n1cc(O)c(=O)[nH]c1=O]

In some embodiments, an opposite nucleobase is.

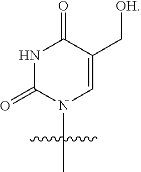 [SMILES: *n1cc(CO)c(=O)[nH]c1=O]

In some embodiments, an opposite nucleobase is

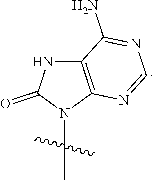 [SMILES: *n1c(=O)[nH]c2c(N)ncnc21]

In some embodiments, an opposite nucleobase is

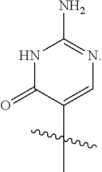 [SMILES: *c1cnc(N)[nH]c1=O]

In some embodiments, an opposite nucleobase is

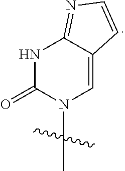 [SMILES: *n1cc2ccnc-2[nH]c1=O]

In some embodiments, an opposite nucleobase is or

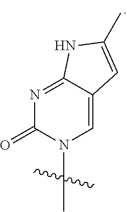 [SMILES: *n1cc2cc(C)[nH]c2nc1=O]

In some embodiments, a second subdomain comprises a modified nucleobase next to an opposite nucleobase. In some embodiments, it is to the 5′ side. In some embodiments, it is to the 3′ side. In some embodiments, on each side there is independently a modified nucleobase. Among other things, the present disclosure recognizes that nucleobases adjacent to (e.g., next to) opposite nucleobases may cause disruption (e.g., steric hindrance) to recognition, binding, interaction, and/or modification of target nucleic acids, oligonucleotides and/or duplexes thereof. In some embodiments, disruption is associated with an adjacent G. In some embodiments, the present disclosure provides nucleobases that can replace G and provide improved stability and/or activities compared to G. For example, in some embodiments, an adjacent nucleobase (e.g., 3′-immediate nucleoside of an opposite nucleoside) is hypoxanthine (replacing G to reduce disruption (e.g., steric hindrance) and/or forming wobble base pairing with C). In some embodiments, an adjacent nucleobase is a derivative of hypoxanthine. In some embodiments, 3′-immediate nucleoside comprises a nucleobase which is or comprise Ring BA having the structure of formula BA-VI. In some embodiments, an adjacent nucleobase is

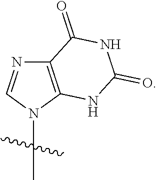 [SMILES: *n1cnc2c(=O)[nH]c(=O)[nH]c21]

In some embodiments, an adjacent nucleobase is

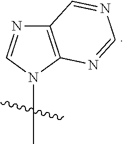 [SMILES: *n1cnc2cncnc21]

In some embodiments, the present disclosure provides flexibility, e.g., with respect to nucleobases that can be utilized in 3′-immediate nucleosides of opposite nucleosides. In some embodiments, the present disclosure provides various modified nucleobases that can be utilized in opposite nucleosides and can allow utilization of G (e.g., opposite to C) in 3′-immediate nucleosides of opposite nucleosides without significantly reducing editing levels. In some embodiments, a nucleobase at N₀ is BA which has the structure of formula BA-III-e as described herein. In some embodiments, a nucleobase at N₀ is BA which has the structure of formula BA-VI as described herein. In some embodiments, N₋₁ comprises hypoxanthine. Certain useful modified nucleobases at adjacent position(s), e.g., N₋₁, and their uses are described in WO 2021/071858, WO 2022/099159, and/or PCT/US2023/018742 and are incorporated herein by reference.

In some embodiments, a second subdomain comprises one or more sugars comprising two 2′-H (e.g., natural DNA sugars). In some embodiments, a second subdomain comprises one or more sugars comprising 2′-OH (e.g., natural RNA sugars). In some embodiments, a second subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2—C3 bond of a corresponding cyclic sugar). In some embodiments, an opposite nucleoside comprises an acyclic sugar such as an UNA sugar. In some embodiments, such an acyclic sugar provides flexibility for proteins to perform modifications on a target adenosine.

In some embodiments, a second subdomain comprises about 1-10 (e.g., about 1, 2, 3, 4, 5, 6, 7, 8, 9, or 10) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a second subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a second subdomain independently comprise a 2′-OR modification, wherein R is optionally substituted C₁₋₆ aliphatic, or a 2′-O-L^{B}-4′ modification. In some embodiments, each sugar in a second subdomain independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a second subdomain independently contains no 2′-OMe.

In some embodiments, high levels (e.g., more than 50%, 60%, 70%, 80%, 90%, or 95%, 99%, or more than 10, 11, 12, 13, 14, 15, 16, 17, 18, 19, 20) of sugars in a second subdomain independently comprise a 2′-OR modification, wherein R is optionally substituted C₁₋₆ aliphatic, or a 2′-O-L^{B}-4′ modification. In some embodiments, each sugar in a second subdomain independently contains a 2′-OR modification, wherein R is optionally substituted C₁₋₆ aliphatic, or a 2′-O-L^{B}-4′ modification, wherein L^{B} is optionally substituted —CH₂—. In some embodiments, each sugar in a second subdomain independently comprises 2′-OMe.

In some embodiments, a second subdomain comprises one or more 2′-F modified sugars.

In some embodiments, a high level (e.g., about 60-100%, or about 60%, 65%, 70%, 75%, 80%, 85%, 90%, 95% or more, or 100%) or all sugars in a second subdomain are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a second subdomain are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a second subdomain are independently 2′-F modified sugars and natural DNA sugars. In some embodiments, a level is 100%. In some embodiments, a second subdomain comprise 1, 2, 3, 4 or 5 2′-F modified sugars. In some embodiments, a second subdomain comprise 1, 2, 3, 4 or 5 sugars comprising two 2′-H. In some embodiments, a second subdomain comprise 1, 2, 3, 4 or 5 natural DNA sugars. In some embodiments, a second subdomain comprise 1, 2, 3, 4 or 5 sugars comprising 2′-OH. In some embodiments, a second subdomain comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, low levels (e.g., no more than 50%, 40%, 30%, 25%, 20%, or 10%, or no more than 1, 2, 3, 4, 5, 6, 7, 8, 9, or 10) of sugars in a second subdomain independently comprise a 2′-F modification. In some embodiments, each sugar in a second subdomain independently contains no 2′-F modification. In some embodiments, each sugar in a second subdomain independently contains no 2′-F.

In some embodiments, sugars of opposite nucleosides to target adenosines (“opposite sugars”), sugars of nucleosides 5′-next to opposite nucleosides (“5′-next sugars”), and/or sugars of nucleosides 3′-next to opposite nucleosides (“3-next sugars”) are independently and optionally 2′-F modified sugars, sugars comprising two 2′-H (e.g., natural DNA sugars), or sugars comprising 2′-OH (e.g., natural RNA sugars). In some embodiments, an opposite sugar is a 2′-F modified sugar. In some embodiments, an opposite sugar is a sugar comprising two 2′-H. In some embodiments, an opposite sugar is a natural DNA sugar. In some embodiments, an opposite sugar is a sugar comprising 2′-OH. In some embodiments, an opposite sugar is a natural RNA sugar. For example, in some embodiments, each of a 5′-next sugar, an opposite sugar and a 3′-next sugar in an oligonucleotide is independently a natural DNA sugar. In some embodiments, a 5′-next sugar is a 2′-F modified sugar, and each of an opposite sugar and a 3′-next sugar is independently a natural DNA sugar.

In some embodiments, a 5′-next sugar is a 2′-F modified sugar. In some embodiments, a 5′-next sugar is a sugar comprising two 2′-H. In some embodiments, a 5′-next sugar is a natural DNA sugar. In some embodiments, a 5′-next sugar is a sugar comprising 2′-OH. In some embodiments, a 5′-next sugar is a natural RNA sugar.

In some embodiments, a 3′-next sugar is a 2′-F modified sugar. In some embodiments, a 3′-next sugar is a sugar comprising two 2′-H. In some embodiments, a 3′-next sugar is a natural DNA sugar. In some embodiments, a 3′-next sugar is a sugar comprising 2′-OH. In some embodiments, a 3′-next sugar is a natural RNA sugar.

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second subdomain comprises 2′-MOE. In some embodiments, no more than about 50% of sugars in a second subdomain comprises 2′-MOE. In some embodiments, no sugars in a second subdomain comprises 2′-MOE.

In some embodiments, a second subdomain comprise about 1-10 (e.g., about 1-5, 1-4, 1-3, about 1, 2, 3, 4, 5, 6, 7, 8, 9, or 10) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%,40%, 50%, 60%, 65%, 70%, 75%, 80%, 85%, 90%, 95%, or 100%, etc.) of internucleotidic linkages in a second subdomain are modified internucleotidic linkages. In some embodiments, each internucleotidic linkage in a second subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-10 (e.g., about 1-5, 1-4, 1-3, about 1, 2, 3, 4, 5, 6, 7, 8, 9, or 10) chiral internucleotidic linkages in a second subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second subdomain is chirally controlled. In some embodiments, each is independently chirally controlled. In some embodiments, at least about 1-10 (e.g., about 1-5, 1-4, 1-3, about 1, 2, 3, 4, 5, 6, 7, 8, 9, or 10) chiral internucleotidic linkages in a second subdomain is Sp. In some embodiments, at least about 1-10 (e.g., about 1-5, 1-4, 1-3, about 1, 2, 3, 4, 5, 6, 7, 8, 9, or 10) phosphorothioate internucleotidic linkages in a second subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%. etc.) of phosphorothioate internucleotidic linkages in a second subdomain is Sp. In some embodiments, the number is one or more. In some embodiments, the number is 2 or more. In some embodiments, the number is 3 or more. In some embodiments, the number is 4 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two second subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a second subdomain is bonded to two nucleosides of the second subdomain. In some embodiments, an internucleotidic linkage bonded to a nucleoside in a second subdomain and a nucleoside in a first or third subdomain may be properly considered an internucleotidic linkage of a second subdomain. In some embodiments, an internucleotidic linkage bonded to a nucleoside in a second subdomain and a nucleoside in a first or third subdomain is a modified internucleotidic linkage; in some embodiments, it is a chiral internucleotidic linkage; in some embodiments, it is chirally controlled; in some embodiments, it is Rp: in some embodiments, it is Sp.

In some embodiments, a second subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a second subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%. 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a second subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a second subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, 1-10 (e.g., about 1-5, 1-4, 1-3, about 1, 2, 3, 4, 5, 6, 7, 8, 9, or 10) internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10. In some embodiments, a second subdomain comprise a higher level (in number and/or percentage) of Rp internucleotidic linkage compared to other portions (e.g., a first domain, a second domain overall, a first subdomain, a third subdomain, or portions thereof). In some embodiments, a second subdomain comprise a higher level (in number and/or percentage) of Rp internucleotidic linkage than Sp internucleotidic linkage.

In some embodiments, each phosphorothioate internucleotidic linkage in a second subdomain is independently chirally controlled. In some embodiments, each is independently Sp or Rp. In some embodiments, a high level is Sp as described herein. In some embodiments, each phosphorothioate internucleotidic linkage in a second subdomain is chirally controlled and is Sp. In some embodiments, one or more, e.g., about 1-5 (e.g., about 1, 2, 3, 4, or 5) is Rp.

In some embodiments, each internucleotidic linkage bonded to a natural DNA or RNA or 2′-F modified sugar in a second subdomain is independently a modified internucleotidic linkage as described herein. In some embodiments, each such modified internucleotidic linkage is independently a phosphorothioate or non-negatively charged internucleotidic linkage such as a phosphoryl guanidine internucleotidic linkage like n001. In some embodiments, each such modified internucleotidic linkage is independently a phosphorothioate or n001 internucleotidic linkage. In some embodiments, each internucleotidic linkage bonded to two second subdomain nucleosides is independently a phosphorothioate internucleotidic linkage. In some embodiments, each phosphorothioate internucleotidic linkage bonded to two second subdomain nucleosides is independently chirally controlled and is Sp. In some embodiments, one or more internucleotidic linkages bonded to a second subdomain nucleoside are independently non-negatively charged internucleotidic linkages such as phosphoryl guanidine internucleotidic linkages like n001. In some embodiments, an internucleotidic linkage bonded to N₋₁ and N₋₂ is an non-negatively charged internucleotidic linkage. In some embodiments, it is a phosphoryl guanidine internucleotidic linkage. In some embodiments, it is n001. In some embodiments, it is chirally controlled and is Rp. In some embodiments, it is chirally controlled and is Sp. In some embodiments, N₋₁ comprises hypoxanthine and in some embodiments, is deoxyinosine. In some embodiments, a phosphoryl guanidine internucleotidic linkage such as n001 bonded to 3′ position of a nucleoside comprising hypoxanthine is chirally controlled and is Sp. In some embodiments, oligonucleotides comprising such Sp phosphoryl guanidine internucleotidic linkages such as Sp n001 bonded to 3′ position of nucleosides comprising hypoxanthine (e.g., deoxyinosine) provide various benefits, e.g., higher activities, better properties, lower manufacturing cost, and/or more readily available manufacturing materials, etc.

In some embodiments, as illustrated in certain examples, a second subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a second subdomain is about 1-5, or about 1, 2, 3, 4, or 5. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a second subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a second subdomain. In some embodiments, the last two or three or four internucleotidic linkages of a second subdomain comprise at least one internucleotidic linkage that is not a non-negatively charged internucleotidic linkage. In some embodiments, the last two or three or four internucleotidic linkages of a second subdomain comprise at least one internucleotidic linkage that is not n001. In some embodiments, the internucleotidic linkage linking the last two nucleosides of a second subdomain is a non-negatively charged internucleotidic linkage. In some embodiments, the internucleotidic linkage linking the last two nucleosides of a second subdomain is a Sp non-negatively charged internucleotidic linkage. In some embodiments, the internucleotidic linkage linking the last two nucleosides of a second subdomain is a Rp non-negatively charged internucleotidic linkage. In some embodiments, the internucleotidic linkage linking the last two nucleosides of a second subdomain is a phosphorothioate internucleotidic linkage. In some embodiments, the internucleotidic linkage linking the last two nucleosides of a second subdomain is a Sp phosphorothioate internucleotidic linkage. In some embodiments, the internucleotidic linkage linking the first two nucleosides of a second subdomain is a non-negatively charged internucleotidic linkage. In some embodiments, the internucleotidic linkage linking the first two nucleosides of a second subdomain is a Sp non-negatively charged internucleotidic linkage. In some embodiments, the internucleotidic linkage linking the first two nucleosides of a second subdomain is a Rp non-negatively charged internucleotidic linkage. In some embodiments, the internucleotidic linkage linking the first two nucleosides of a second subdomain is a phosphorothioate internucleotidic linkage. In some embodiments, the internucleotidic linkage linking the first two nucleosides of a second subdomain is a Sp phosphorothioate internucleotidic linkage. In some embodiments, the internucleotidic linkage linking the last nucleoside of a second subdomain and the first nucleoside of a third subdomain is a non-negatively charged internucleotidic linkage. In some embodiments, the internucleotidic linkage linking the last nucleoside of a second subdomain and the first nucleoside of a third subdomain is a Sp non-negatively charged internucleotidic linkage. In some embodiments, the internucleotidic linkage linking the last nucleoside of a second subdomain and the first nucleoside of a third subdomain is a Rp non-negatively charged internucleotidic linkage. In some embodiments, the internucleotidic linkage linking the last nucleoside of a second subdomain and the first nucleoside of a third subdomain is a phosphorothioate internucleotidic linkage. In some embodiments, the internucleotidic linkage linking the last nucleoside of a second subdomain and the first nucleoside of a third subdomain is a Sp phosphorothioate internucleotidic linkage. In some embodiments, a non-negatively charged internucleotidic linkage is a neutral internucleotidic linkage such as n001.

In some embodiments, a second subdomain comprises one or more natural phosphate linkages. In some embodiments, a second subdomain contains no natural phosphate linkages. In some embodiments, a second subdomain comprises at least 1 natural phosphate linkage. In some embodiments, a second subdomain comprises at least 2 natural phosphate linkages. In some embodiments, a second subdomain comprises at least 3 natural phosphate linkages. In some embodiments, a second subdomain comprises at least 4 natural phosphate linkages. In some embodiments, a second subdomain comprises at least 5 natural phosphate linkages.

In some embodiments, an opposite nucleoside is connected to its 5′ immediate nucleoside through a natural phosphate linkage. In some embodiments, an opposite nucleoside is connected to its 5′ immediate nucleoside through a natural phosphate linkage. In some embodiments, an opposite nucleoside is connected to its 5′ immediate nucleoside through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp.

In some embodiments, an opposite nucleoside is connected to its 3′ immediate nucleoside (-1 position relative to the opposite nucleoside) through a natural phosphate linkage. In some embodiments, an opposite nucleoside is connected to its 3′ immediate nucleoside through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n00l) and is not chirally controlled.

In some embodiments, a nucleoside at -1 position relative to an opposite nucleoside and a nucleoside at −2 position relative to an opposite nucleoside (e.g., in 5′- . . . N₀N₋₁N₋₂ . . . 3′, if N₀ is an opposite nucleoside, N₋₁ is at −1 position and N₋₂ is at −2 position) is linked through a natural phosphate linkage. In some embodiments, they are connected through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n001) and is not chirally controlled.

In some embodiments, a nucleoside of a second subdomain and a nucleoside of a third subdomain is linked through a natural phosphate linkage. In some embodiments, they are connected through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n001) and is not chirally controlled.

In some embodiments, an oligonucleotide comprises 5′-N₁N₀N₋₁-3′, wherein each of N₁, N₀, and N₋₁ is independently a nucleoside, N₁ and N₀ bond to an internucleotidic linkage as described herein, and N₋₁ and N₀ bond to an internucleotidic linkage as described herein, and N₀ is opposite to a target adenosine. In some embodiments, the sugar of each of N₁, N₀, and N₋₁ is independently a natural DNA sugar or a 2′-F modified sugar. In some embodiments, the sugar of each of N₁, N₀, and N₋₁ is independently a natural DNA sugar. In some embodiments, the sugar of N₁ is a 2′-modified sugar, and the sugar of each of N₀ and N₋₁ is independently a natural DNA sugar. In some embodiments, such oligonucleotides provide high editing levels. In some embodiments, each of the two internucleotidic linkages bonded to N₋₁ is independently Rp. In some embodiments, each of the two internucleotidic linkages bonded to N₋₁ is independently an Rp phosphorothioate internucleotidic linkage. In some embodiments, each of the two internucleotidic linkages bonded to N₋₁ is independently an Rp phosphorothioate internucleotidic linkage, and each other phosphorothioate internucleotidic linkage in an oligonucleotide, if any, is independently Sp. In some embodiments, a 5′ internucleotidic linkage bonded to N₁ is Rp. In some embodiments, an internucleotidic linkage bonded to N₁ and N₀ (i.e., a 3′ internucleotidic linkage bonded to N₁) is Rp. In some embodiments, an internucleotidic linkage bonded to N₋₁ and N₀ is Rp. In some embodiments, a 3′ internucleotidic linkage bonded to N₋₁ is Rp. In some embodiments, each internucleotidic linkage bonded to N₀ is independently Rp. In some embodiments, each internucleotidic linkage bonded to N₀ or N₁ is independently Rp. In some embodiments, each internucleotidic linkage bonded to N₀ or N₋₁ is independently Rp. In some embodiments, each internucleotidic linkage bonded to N₁ is independently Rp. In some embodiments, each Rp internucleotidic linkage is independently an Rp phosphorothioate internucleotidic linkage. In some embodiments, each other chirally controlled phosphorothioate internucleotidic linkage in an oligonucleotide is independently Sp.

In some embodiments, sugar of a 5′ immediate nucleoside (e.g., N₁) is independently selected from a natural DNA sugar, a natural RNA sugar, and a 2′-F modified sugar (e.g., R^{2s} is —F). In some embodiments, sugar of an opposite nucleoside (e.g., N₀) is independently selected from a natural DNA sugar, a natural RNA sugar, and a 2′-F modified sugar. In some embodiments, sugar of a 3′ immediate nucleoside (e.g., N₋₁) is independently selected from a natural DNA sugar, a natural RNA sugar, and a 2′-F modified sugar. In some embodiments, sugars of a 5′ immediate nucleoside, an opposite nucleoside, and a 3′ immediate nucleoside are each independently a natural DNA sugar. In some embodiments, sugars of a 5′ immediate nucleoside, an opposite nucleoside, and a 3′ immediate nucleoside are a natural DNA sugar, a natural RNA sugar, and natural DNA sugar, respectively. In some embodiments, sugars of a 5′ immediate nucleoside, an opposite nucleoside, and a 3′ immediate nucleoside are a 2′-F modified sugar, a natural RNA sugar, and natural DNA sugar, respectively.

In some embodiments, sugar of an opposite nucleoside is a natural RNA sugar. In some embodiments, such an opposite nucleoside is utilized with a 3′ immediate I nucleoside (which is optionally complementary to a C in a target nucleic acid when aligned). In some embodiments, an internucleotidic linkage between the 3′ immediate nucleoside (e.g., N⁻¹) and its 3′ immediate nucleoside (e.g., N₋₂) is a non-negatively charged internucleotidic linkage, e.g., n001. In some embodiments, it is stereorandom. In some embodiments, it is chirally controlled and is Rp. In some embodiments, it is chirally controlled and is Sp.

In some embodiments, an internucleotidic linkage that is bonded to a 3′ immediate nucleoside (e.g., N₋₁) and its 3′ neighboring nucleoside (e.g., N₋₂ in 5′-N₁N₀N₋₁N₋₂-3′) is a modified internucleotidic linkage. In some embodiments, it is a chiral internucleotidic linkage. In some embodiments, it is stereorandom. In some embodiments, it is a stereorandom phosphorothioate internucleotidic linkage. In some embodiments, it is a stereorandom non-negatively charged internucleotidic linkage. In some embodiments, it is stereorandom n001. In some embodiments, it is chirally controlled. In some embodiments, it is a Rp phosphorothioate internucleotidic linkage. In some embodiments, it is a Sp phosphorothioate internucleotidic linkage. In some embodiments, it is chirally controlled. In some embodiments, it is a Rp non-negatively charged internucleotidic linkage. In some embodiments, it is a Sp non-negatively charged internucleotidic linkage. In some embodiments, a non-negatively charged internucleotidic linkage is a neutral internucleotidic linkage. In some embodiments, a non-negatively charged internucleotidic linkage is n001.

In some embodiments, N₋₁ is I. In some embodiments, I is utilized replacing G, e.g., when a target nucleic acid comprises 5′-CA-3′ wherein A is a target adenosine. In some embodiments, 5′-N₁N₀N₋₁-3′is 5′-N₁N₀I-3′. In some embodiments, N₀ is b001A, b002A, b003A, b008U, b001C, C, A, or U. In some embodiments, N₀ is b001A, b002A, b008U, b001C, C. or A. In some embodiments, N₀ is b001A, b002A, b008U, or b001C. In some embodiments, N₀ is b001A. In some embodiments, N₀ is b002A. In some embodiments, N₀ is b003A. In some embodiments, N₀ is b008U. In some embodiments, N₀ is b001C. In some embodiments, N₀ is A. In some embodiments, N₀ is U.

In some embodiments provided oligonucleotides comprising certain nucleobases (e.g., b001A, b002A, b008U, C, A, etc.) opposite to target adenosines can among other things provide improved editing efficiency (e.g., compared to a reference nucleobase such as U). In some embodiments, an opposite nucleoside is linked to an I to its 3′ side.

In some embodiments, a second subdomain comprises an editing region as described herein.

In some embodiments, a second subdomain comprises a 5′-end portion, e.g., one having a length of about 1-5, 1-3, or 1, 2, 3, 4, or 5 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-5, 1-3, or 1, 2, 3, 4, or 5) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a 5′-end portion independently comprise a 2′-OR modification, wherein R is optionally substituted C₁₋₆ aliphatic, or a 2′-O-L^{B}-4′ modification. In some embodiments, each sugar in a 5′-end portion independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a 5′-end portion independently contains no 2′-OMe.

In some embodiments, a 5′-end portion comprises one or more 2′-F modified sugars.

In some embodiments, a high level (e.g., about 60-100%, or about 60%, 65%, 70%, 75%, 80%, 85%, 90%, 95% or more, or 100%) or all sugars in a 5′-end are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a 5′-end portion are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a 5′-end portion are independently 2′-F modified sugars and natural DNA sugars. In some embodiments, a level is 100%. In some embodiments, sugars of a 5′-end portion are selected from sugars having two 2′-H (e.g., natural DNA sugar) and 2′-F modified sugars. In some embodiments, a 5′-end portion comprise 1, 2, 3, 4 or 5 2′-F modified sugars. In some embodiments, a 5′-end portion comprise 1, 2, 3, 4 or 5 sugars comprising two 2′-H. In some embodiments, a 5′-end portion comprise 1, 2, 3, 4 or 5 natural DNA sugars. In some embodiments, a 5′-end portion comprise 1, 2, 3, 4 or 5 sugars comprising 2′-OH. In some embodiments, a 5′-end portion comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, one or more (e.g., about 1, 2, 3, 4, or 5) internucleotidic linkages of a 5′-end portion are independently a modified internucleotidic linkage. In some embodiments, one or more (e.g., about 1, 2, 3, 4, or 5) internucleotidic linkages of a 5′-end portion are independently a chiral internucleotidic linkage. In some embodiments, one or more (e.g., about 1, 2, 3, 4, or 5) internucleotidic linkages of a 5′-end portion are independently a chirally controlled internucleotidic linkage. In some embodiments, one or more (e.g., about 1, 2, 3, 4, or 5) internucleotidic linkages of a 5′-end portion are Rp. In some embodiments, one or more (e.g., about 1, 2, 3, 4, or 5) internucleotidic linkages of a 5′-end portion are Sp. In some embodiments, each internucleotidic linkage of a 5′-end portion is Sp.

In some embodiments, one or more (e.g., about 1, 2, 3, 4, or 5) internucleotidic linkages of a 5′-end portion are independently a modified internucleotidic linkage. In some embodiments, one or more (e.g., about 1, 2, 3, 4, or 5) internucleotidic linkages of a 5′-end portion are independently a chiral internucleotidic linkage. In some embodiments, one or more (e.g., about 1, 2, 3, 4, or 5) internucleotidic linkages of a 5′-end portion are independently a chirally controlled internucleotidic linkage. In some embodiments, one or more (e.g., about 1, 2, 3, 4, or 5) internucleotidic linkages of a 5′-end portion are Rp. In some embodiments, one or more (e.g., about 1, 2, 3, 4, or 5) internucleotidic linkages of a 5′-end portion are Rp. In some embodiments, each internucleotidic linkage of a 5′-end portion is Rp.

In some embodiments, a 5′-end portion comprises one or more (e.g., about 1, 2, 3, 4, or 5) mismatches as described herein. In some embodiments, a 5′-end portion comprises one or more (e.g., about 1, 2, 3, 4, or 5) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a 5′-end portion comprises a nucleoside 5′ next to an opposite nucleoside. In some embodiments, a nucleoside 5′ next to an opposite nucleoside comprise a nucleobase as described herein.

In some embodiments, a second subdomain comprises a 3′-end portion, e.g., one having a length of about 1-5, 1-3, or 1, 2, 3, 4, or 5 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a second subdomain consists a 5′-end portion and a 3′-end portion.

In some embodiments, a 3′-end portion comprises one or more sugars having two 2′-H (e.g., natural DNA sugars). In some embodiments, a 3′-end portion comprises one or more sugars having 2′-OH (e.g., natural RNA sugars). In some embodiments, one or more (e.g., about 1-5, 1-3, or 1, 2, 3, 4, or 5) of sugars in a 3′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a 3′-end portion independently comprise a 2′-OR modification, wherein R is optionally substituted C₁₆ aliphatic, or a 2′-O-L^{B}-4′ modification. In some embodiments, each sugar in a 3′-end portion independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a 3′-end portion independently contains no 2′-OMe.

In some embodiments, a 3′-end portion comprises one or more 2′-F modified sugars.

In some embodiments, a high level (e.g., about 60-100%, or about 60%, 65%, 70%, 75%, 80%, 85%, 90%, 95% or more, or 100%) or all sugars in a 3′-end are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a 3′-end portion are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a 3′-end portion are independently 2′-F modified sugars and natural DNA sugars. In some embodiments, a level is 100%. In some embodiments, sugars of a 3′-end portion are selected from sugars having two 2′-H (e.g., natural DNA sugar) and 2′-F modified sugars. In some embodiments, a 3′-end portion comprise 1, 2, 3, 4 or 5 2′-F modified sugars. In some embodiments, a 3′-end portion comprise 1, 2, 3, 4 or 5 sugars comprising two 2′-H. In some embodiments, a 3′-end portion comprise 1, 2, 3, 4 or 5 natural DNA sugars. In some embodiments, a 3′-end portion comprise 1, 2, 3, 4 or 5 sugars comprising 2′-OH. In some embodiments, a 3′-end portion comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, one or more (e.g., about 1, 2, 3, 4, or 5) internucleotidic linkages of a 3′-end portion are independently a modified internucleotidic linkage. In some embodiments, one or more (e.g., about 1, 2, 3, 4, or 5) internucleotidic linkages of a 3′-end portion are independently a chiral internucleotidic linkage. In some embodiments, one or more (e.g., about 1, 2, 3, 4, or 5) internucleotidic linkages of a 3′-end portion are independently a chirally controlled internucleotidic linkage. In some embodiments, one or more (e.g., about 1, 2, 3, 4, or 5) internucleotidic linkages of a 3′-end portion are Rp. In some embodiments, one or more (e.g., about 1, 2, 3, 4, or 5) internucleotidic linkages of a 3′-end portion are Sp. In some embodiments, each internucleotidic linkage of a 3′-end portion is Sp.

In some embodiments, one or more (e.g., about 1, 2, 3, 4, or 5) internucleotidic linkages of a 3′-end portion are independently a modified internucleotidic linkage. In some embodiments, one or more (e.g., about 1, 2, 3, 4, or 5) internucleotidic linkages of a 3′-end portion are independently a chiral internucleotidic linkage. In some embodiments, one or more (e.g., about 1, 2, 3, 4, or 5) internucleotidic linkages of a 3′-end portion are independently a chirally controlled internucleotidic linkage. In some embodiments, one or more (e.g., about 1, 2, 3, 4, or 5) internucleotidic linkages of a 3′-end portion are Rp. In some embodiments, one or more (e.g., about 1, 2, 3, 4, or 5) internucleotidic linkages of a 3′-end portion are Rp. In some embodiments, each internucleotidic linkage of a 3′-end portion is Rp.

In some embodiments, a 3′-end portion comprises one or more (e.g., about 1, 2, 3, 4, or 5) mismatches as described herein. In some embodiments, a 3′-end portion comprises one or more (e.g., about 1, 2, 3, 4, or 5) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a 3′-end portion comprises a nucleoside 3′ next to an opposite nucleoside. In some embodiments, a nucleoside 3′ next to an opposite nucleoside comprise a nucleobase as described herein. In some embodiments, a nucleoside 3′ next to an opposite nucleoside forms a wobble pair with a corresponding nucleoside in a target nucleic acid. In some embodiments, the nucleobase of a nucleoside 3′ next to an opposite nucleoside is hypoxanthine; in some embodiments, it is a derivative of hypoxanthine.

In some embodiments, a second subdomain recruits, promotes or contribute to recruitment of, a protein such as an ADAR protein, e.g., ADAR1, ADAR2, etc. In some embodiments, a second subdomain recruits, or promotes or contribute to interactions with, a protein such as an ADAR protein. In some embodiments, a second subdomain contacts with a RNA binding domain (RBD) of ADAR. In some embodiments, a second subdomain contacts with a catalytic domain of ADAR which has a deaminase activity. In some embodiments, a second subdomain contact with a domain that has a deaminase activity of ADAR1. In some embodiments, a second subdomain contact with a domain that has a deaminase activity of ADAR2. In some embodiments, various nucleobases, sugars and/or internucleotidic linkages of a second subdomain may interact with one or more residues of proteins, e.g., ADAR proteins.

### Third Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a third subdomain are described below as examples.

In some embodiments, a third subdomain has a length of about 1-50, 1-40, 1-30, 1-20 (e.g., about 1, 2, 3, 4, 5, 6, 7, 8, 9, or 10-about 10, 11, 12, 13, 14, 15, 16, 17, 18. 19, 20, 21, 22, 23, 24, 25, 30, 40 or 50, or about 1, 2, 3, 4, 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a third subdomain has a length of about 5-30 nucleobases. In some embodiments, a third subdomain has a length of about 10-30 nucleobases. In some embodiments, a third subdomain has a length of about 10-20 nucleobases. In some embodiments, a third subdomain has a length of about 5-15 nucleobases. In some embodiments, a third subdomain has a length of about 13-16 nucleobases. In some embodiments, a third subdomain has a length of about 6-12 nucleobases. In some embodiments, a third subdomain has a length of about 6-9 nucleobases. In some embodiments, a third subdomain has a length of about 1-10 nucleobases. In some embodiments, a third subdomain has a length of about 1-7 nucleobases. In some embodiments, a third subdomain has a length of 1 nucleobase. In some embodiments, a third subdomain has a length of 2 nucleobases. In some embodiments, a third subdomain has a length of 3 nucleobases. In some embodiments, a third subdomain has a length of 4 nucleobases. In some embodiments, a third subdomain has a length of 5 nucleobases. In some embodiments, a third subdomain has a length of 6 nucleobases. In some embodiments, a third subdomain has a length of 7 nucleobases. In some embodiments, a third subdomain has a length of 8 nucleobases. In some embodiments, a third subdomain has a length of 9 nucleobases. In some embodiments, a third subdomain has a length of 10 nucleobases. In some embodiments, a third subdomain has a length of 11 nucleobases. In some embodiments, a third subdomain has a length of 12 nucleobases. In some embodiments, a third subdomain has a length of 13 nucleobases. In some embodiments, a third subdomain has a length of 14 nucleobases. In some embodiments, a third subdomain has a length of 15 nucleobases. In some embodiments, a third subdomain is shorter than a first subdomain. In some embodiments, a third subdomain is shorter than a first domain. In some embodiments, a third subdomain comprises a 3′-end nucleobase of a second domain.

In some embodiments, a third subdomain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of a second domain. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%. In some embodiments, the 5′-end nucleoside of a third subdomain is N₋₂. In some embodiments, all nucleosides from N₋₂ to the 3′-end are in a third subdomain.

In some embodiments, one or more (e.g., 1-20, 1, 2, 3, 4, 5, 6, 7, 8, 9, or 10, etc.) mismatches exist in a third subdomain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a third subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a third subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a third subdomain is fully complementary to a target nucleic acid.

In some embodiments, a third subdomain comprises one or more modified nucleobases.

In some embodiments, a third subdomain comprises a nucleoside opposite to a target adenosine (an opposite nucleoside). In some embodiments, a third subdomain comprises a nucleoside 3′ next to an opposite nucleoside. In some embodiments, a third subdomain comprises a nucleoside 5′ next to an opposite nucleoside. Various suitable opposite nucleosides, including sugars and nucleobases thereof, have been described herein.

In some embodiments, a third subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA.

In some embodiments, a third subdomain comprises one or more sugars comprising two 2′-H (e.g., natural DNA sugars). In some embodiments, a third subdomain comprises one or more sugars comprising 2′-OH (e.g., natural RNA sugars). In some embodiments, a third subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) RNA sugars. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) DNA sugars.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a 2′-OR modified sugar, wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl. In some embodiments, N₋₂ comprises a 2′-OR modified sugar wherein R is not —H. In some embodiments, N₋₃ comprises a 2′-F modified sugar. In some embodiments, each nucleoside after N₋₃ independently comprises a 2′-OR modified sugar wherein R is not —H. In some embodiments, N₋₃ comprises a 2′-F modified sugar and each other nucleosides in a third subdomain independently comprises a 2′-OR modified sugar wherein R is not —H. In some embodiments, a 2′-OR modified sugar is independently a 2′-OMe or 2′-MOE modified sugar. In some embodiments, 2′-OR modified sugar is independently a 2′-OMe modified sugar. In some embodiments, each 2′-OR modified sugar is independently a 2′-ORm odified sugar wherein R is optionally substituted C₁₋₆ aliphatic or a bicyclic sugar. In some embodiments, each 2′-OR modified sugar is independently a 2′-OR modified sugar wherein R is optionally substituted C₁₋₆ aliphatic. In some embodiments, each 2′-OR modified sugar is independently a 2′-OMe or 2′-MOE modified sugar. In some embodiments, each 2′-OR modified sugar is independently a 2′-OMe modified sugar.

In some embodiments, a third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a third subdomain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a third subdomain are independently modified sugars with a modification that is not 2′-F. In some embodiments, modified sugars of a third subdomain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, a third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%. 75%-100%, 80%-85%, 80%-90%, 80%-95%, 80%-100%, 85%-90%, 85%-95%, 85%-100%, 90%-95%, 90%-100%, 10%, 20%, 30%, 40%, 5 60%, 65%, 70%, 75%, 80%, 85%, 90%, 95%, or 100%, etc.) of sugars in a third subdomain are independently modified sugars selected from a bicyclic sugar (e.g., a LNA sugar), an acyclic sugar (e.g., a UNA sugar), a sugar with a 2′-OR modification, or a sugar with a 2′-N(R)2 modification, wherein each R is independently optionally substituted C₁₋₆ aliphatic. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, each sugar in a third subdomain independently comprises a 2′-OR modification, wherein R is optionally substituted C₁₋₆ aliphatic, or a 2′-O-L^{B}-4′ modification. In some embodiments, each sugar in a third subdomain independently comprises a 2′-OR modification, wherein R is optionally substituted C₁₋₆ aliphatic, or a 2′-O-L^{B}-4′ modification, wherein L^{B} is optionally substituted —CH₂—. In some embodiments, each sugar in a third subdomain independently comprises 2′-OMe.

In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) 2′-F modified sugars. In some embodiments, a third subdomain comprises no 2′-F modified sugars. In some embodiments, a third subdomain comprises one or more bicyclic sugars and/or 2′-OR modified sugars wherein R is not —H. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a third subdomain comprises 2′-F. In some embodiments, no more than about 50% of sugars in a third subdomain comprises 2′-F. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a third subdomain comprises 2′-MOE. In some embodiments, no more than about 50% of sugars in a third subdomain comprises 2′-MOE. In some embodiments, no sugars in a third subdomain comprises 2′-MOE.

In some embodiments, a third subdomain comprise about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10. 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a third subdomain are modified internucleotidic linkages. In some embodiments, each internucleotidic linkage in a third subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a third subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a third subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a third subdomain is chirally controlled. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a third subdomain is Sp. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a third subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a third subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%0, 90%, 95%, or 100%, etc.) of phosphorothioate internucleotidic linkages in a third subdomain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two third subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a third subdomain is bonded to two nucleosides of the third subdomain. In some embodiments, an internucleotidic linkage bonded to a nucleoside in a third subdomain and a nucleoside in a second subdomain may be properly considered an internucleotidic linkage of a third subdomain. In some embodiments, an internucleotidic linkage bonded to a nucleoside in a third subdomain and a nucleoside in a second subdomain is a modified internucleotidic linkage; in some embodiments, it is a chiral internucleotidic linkage; in some embodiments, it is chirally controlled; in some embodiments, it is Rp; in some embodiments, it is Sp.

In some embodiments, a third subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a third subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%. 65%-90%, 65%-95%, 65%-100%, 70%-80%, 70%-85%, 70%-90%, 70%-95%, 70%-100%, 75%-80%, 75%-85%. 75%-90%, 75%-95%, 75%-100%, 80%-85%, 80%-90%, 80%-95%, 80%-100%, 85%-90%, 85%-95%. 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a third subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a third subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a third subdomain is independently chirally controlled. In some embodiments, each is independently Sp or Rp. In some embodiments, a high level is Sp as described herein. In some embodiments, each phosphorothioate internucleotidic linkage in a third subdomain is chirally controlled and is Sp. In some embodiments, one or more, e.g., about 1-5 (e.g., about 1, 2, 3, 4, or 5) is Rp.

In some embodiments, as illustrated in certain examples, a third subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a third subdomain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments. no two non-negatively charged internucleotidic linkages are consecutive. In some embodiments, all non-negatively charged internucleotidic linkages in a third subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a third subdomain. In some embodiments, the last two or three or four internucleotidic linkages of a third subdomain comprise at least one internucleotidic linkage that is not a non-negatively charged internucleotidic linkage. In some embodiments, the last two or three or four internucleotidic linkages of a third subdomain comprise at least one internucleotidic linkage that is not n001. In some embodiments, the internucleotidic linkage linking the last two nucleosides of a third subdomain is a non-negatively charged internucleotidic linkage. In some embodiments, the internucleotidic linkage linking the last two nucleosides of a third subdomain is a Sp non-negatively charged internucleotidic linkage. In some embodiments, the internucleotidic linkage linking the last two nucleosides of a third subdomain is a Rp non-negatively charged internucleotidic linkage. In some embodiments, the internucleotidic linkage linking the last two nucleosides of a third subdomain is a phosphorothioate internucleotidic linkage. In some embodiments, the internucleotidic linkage linking the last two nucleosides of a third subdomain is a Sp phosphorothioate internucleotidic linkage. In some embodiments, the last two nucleosides of a third subdomain are the last two nucleosides of a second domain. In some embodiments, the last two nucleosides of a third subdomain are the last two nucleosides of an oligonucleotide. In some embodiments, the internucleotidic linkage linking the first two nucleosides of a third subdomain is a non-negatively charged internucleotidic linkage. In some embodiments, the internucleotidic linkage linking the first two nucleosides of a third subdomain is a Sp non-negatively charged internucleotidic linkage. In some embodiments, the internucleotidic linkage linking the first two nucleosides of a third subdomain is a Rp non-negatively charged internucleotidic linkage. In some embodiments, the internucleotidic linkage linking the first two nucleosides of a third subdomain is a phosphorothioate internucleotidic linkage. In some embodiments, the internucleotidic linkage linking the first two nucleosides of a third subdomain is a Sp phosphorothioate internucleotidic linkage. In some embodiments, a non-negatively charged internucleotidic linkage is a neutral internucleotidic linkage such as n001. In some embodiments, it is chirally controlled and is Rp. In some embodiments, the last and/or the second last internucleotidic linkage of an oligonucleotide is a non-negatively charged internucleotidic linkage such as a phosphoryl guanidine internucleotidic linkage like n001. In some embodiments, it is chirally controlled and is Rp.

In some embodiments, a third subdomain comprises one or more (e.g., about 1, 2, 3, 4, 5, 6, 7, 8, 9, 10) natural phosphate linkages. In some embodiments, a third subdomain contains no natural phosphate linkages. In some embodiments, the internucleotidic linkage bonded to N₋₂ and N⁻³ is a natural phosphate linkage. In some embodiments, sugar of N₋₃ is a 2′-F modified sugar and sugar of N₋₂ is a 2′-OR modified sugar wherein R is not —H (e.g., a 2′-OMe modified sugar). In some embodiments, among all internucleotidic linkages bonded to two nucleosides of a third subdomain, one is a natural phosphate linkage (e.g., between N₋₂ and N₋₃ as described herein), one is a Rp non-negatively charged internucleotidic linkage such as a phosphoryl guanidine internucleotidic linkage n001 (e.g., the last or the second last internucleotidic linkage of an oligonucleotide), and all the others are Sp phosphorothioate internucleotidic linkages.

In some embodiments, a third subdomain comprises a 5′-end portion, e.g., one having a length of about 1-20, 1-15, 1-10, 1-8, 1-5, 1-3, 3-8, or about 1, 2, 3, 4, 5, 6, 7, 8, 9, or 10 nucleobases. In some embodiments, a 5′-end portion has a length of about 1-3 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 5′-end portion comprises the 5′-end nucleobase of a third subdomain. In some embodiments, a third subdomain comprises or consists of a 3′-end portion and a 5′-end portion. In some embodiments, a 5′-end portion comprises the 5′-end nucleobase of a third subdomain. In some embodiments, a 5′-end portion of a third subdomain is bonded to a second subdomain.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, compared to a 3′-end portion, 5′ end portion contains a higher level (in numbers and/or percentage) of 2′-F modified sugars and/or sugars comprising two 2′-H (e.g., natural DNA sugars), and/or a lower level (in numbers and/or percentage) of other types of modified sugars, e.g., bicyclic sugars and/or sugars with 2′-OR modifications wherein R is independently optionally substituted C₁₆ aliphatic. In some embodiments, compared to a 3′-end portion, a 5′-end portion contains a higher level of 2′-F modified sugars and/or a lower level of 2′-OR modified sugars wherein R is optionally substituted C₁₋₆ aliphatic. In some embodiments, compared to a 3′-end portion, a 5′-end portion contains a higher level of 2′-F modified sugars and/or a lower level of 2′-OMe modified sugars. In some embodiments, compared to a 3′-end portion, a 5′-end portion contains a higher level of natural DNA sugars and/or a lower level of 2′-OR modified sugars wherein R is optionally substituted C₁₋₆ aliphatic. In some embodiments, compared to a 3′-end portion, a 5′-end portion contains a higher level of natural DNA sugars and/or a lower level of 2′-OMe modified sugars. In some embodiments, a 5′-end portion contains low levels (e.g., no more than 50%, 40%, 30%, 25%, 20%, or 10%, or no more than 1, 2, 3, 4, 5, 6, 7, 8, 9, or 10) of modified sugars which are bicyclic sugars or sugars comprising 2′-OR wherein R is optionally substituted C₁₋₆ aliphatic (e.g., methyl). In some embodiments, a 5′-end portion contains no modified sugars which are bicyclic sugars or sugars comprising 2′-OR wherein R is optionally substituted C₁₋₆ aliphatic (e.g., methyl).

In some embodiments, one or more modified sugars independently comprise 2′-F. In some embodiments, no modified sugars comprises 2′-OMe or other 2′-OR modifications wherein R is optionally substituted C₁₋₆ aliphatic. In some embodiments, each sugar of a 5′-end portion independently comprises two 2′-H or a 2′-F modification. In some embodiments, a 5′-end portion comprises 1, 2, 3, 4, or 5 2′-F modified sugars. In some embodiments, a 5′-end portion comprises 1-3 2′-F modified sugars. In some embodiments, a 5′-end portion comprises 1, 2, 3, 4, or 5 natural DNA sugars. In some embodiments, a 5′-end portion comprises 1-3 natural DNA sugars.

In some embodiments, one or more (e.g., about 1-10, or about 1, 2, 3, 4, 5, 6, 7, 8, 9, or 10) internucleotidic linkages of a 5′-end portion are independently a modified internucleotidic linkage. In some embodiments, one or more (e.g., about 1-10, or about 1, 2, 3, 4, 5, 6, 7, 8, 9, or 10) internucleotidic linkages of a 5′-end portion are independently a chiral internucleotidic linkage. In some embodiments, one or more (e.g., about 1-10, or about 1, 2, 3, 4, 5, 6, 7, 8, 9, or 10) internucleotidic linkages of a 5′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 5′-end portion are Rp. In some embodiments, one or more (e.g., about 1-10, or about 1, 2, 3, 4, 5, 6, 7, 8, 9, or 10) internucleotidic linkages of a 5′-end portion are Sp. In some embodiments, each internucleotidic linkage of a 5′-end portion is Sp. In some embodiments, a 5′-end portion contains a higher level (in number and/or percentage) of Rp internucleotidic linkage and/or natural phosphate linkage compared to a 3′-end portion.

In some embodiments, a 5′-end portion comprises one or more (e.g., about 1-10, or about 1, 2, 3, 4, 5, 6, 7, 8, 9, or 10) mismatches as described herein. In some embodiments, a 5′-end portion comprises one or more (e.g., about 1-10, or about 1, 2, 3, 4, 5, 6, 7, 8, 9, or 10) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a third subdomain comprises a 3′-end portion, e.g., one having a length of about 1-20, 1-15, 1-10, 1-8, 1-4, 3-8, or about 1, 2, 3, 4, 5, 6, 7, 8, 9, or 10 nucleobases. In some embodiments, a 3′-end portion has a length of about 3-6 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 3′-end portion comprises the 3′-end nucleobase of a third subdomain.

In some embodiments, a 3′-end portion comprises one or more sugars having two 2′-H (e.g., natural DNA sugars). In some embodiments, a 3′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 3′-end portion are independently modified sugars. In some embodiments, about 5%-100% (e.g., about 10%-100%, 20-100%, 30%-100%, 40%-100%,50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 3′-end portion is independently a bicyclic sugar (e.g., a LNA sugar) or a sugar with a 2′-OR modification wherein R is optionally substituted C₁₋₆ aliphatic. In some embodiments, each modified sugar in a 3′-end portion is independently a bicyclic sugar (e.g., a LNA sugar) or a sugar with a 2′-OR modification wherein R is optionally substituted C₁₋₆ aliphatic. In some embodiments, each modified sugar in a 3′-end portion is independently a sugar with a 2′-OR modification wherein R is optionally substituted C₁₋₆ aliphatic. In some embodiments, R is methyl.

In some embodiments, one or more sugars in a 3′-end portion independently comprise a 2′-OR modification, wherein R is optionally substituted C₁₆ aliphatic, or a 2′-O-L^{B}-4′ modification. In some embodiments, each sugar in a 3′-end portion independently comprises a 2′-OR modification, wherein R is optionally substituted C₁₋₆ aliphatic, or a 2′-O-L^{B}-4′ modification. In some embodiments, L^{B} is optionally substituted —CH₂—. In some embodiments, L^{B} is —CH₂—. In some embodiments, each sugar in a 3′-end portion independently comprises 2′-OMe.

In some embodiments, one or more (e.g., about 1-10, or about 1, 2, 3, 4, 5, 6, 7, 8, 9, or 10) internucleotidic linkages of a 3′-end portion are independently a modified internucleotidic linkage. In some embodiments, one or more (e.g., about 1-10, or about 1, 2, 3, 4, 5, 6, 7, 8, 9, or 10) internucleotidic linkages of a 3′-end portion are independently a chiral internucleotidic linkage. In some embodiments, one or more (e.g., about 1-10, or about 1, 2, 3, 4, 5, 6, 7, 8, 9, or 10) internucleotidic linkages ofa 3′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 3′-end portion are Rp. In some embodiments, one or more (e.g., about 1-10, or about 1, 2, 3, 4, 5, 6, 7, 8, 9, or 10) internucleotidic linkages of a 3′-end portion are Sp. In some embodiments, each internucleotidic linkage of a 3′-end portion is Sp.

In some embodiments, a 3′-end portion comprises one or more (e.g., about 1-10, or about 1, 2, 3, 4, 5, 6, 7, 8, 9, or 10) mismatches as described herein. In some embodiments, a 3′-end portion comprises one or more (e.g., about 1-10, or about 1, 2, 3, 4, 5, 6, 7, 8, 9, or 10) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a third subdomain recruits, promotes or contribute to recruitment of, a protein such as an ADAR protein, e.g., ADAR1, ADAR2, etc. In some embodiments, a third subdomain recruits, or promotes or contribute to interactions with, a protein such as an ADAR protein. In some embodiments, a third subdomain contacts with a RNA binding domain (RBD) of ADAR. In some embodiments, a third subdomain contacts with a catalytic domain of ADAR which has a deaminase activity. In some embodiments, a third subdomain contact with a domain that has a deaminase activity of ADAR1. In some embodiments, a third subdomain contact with a domain that has a deaminase activity of ADAR2. In some embodiments, various nucleobases, sugars and/or internucleotidic linkages of a third subdomain may interact with one or more residues of proteins, e.g., ADAR proteins.

In some embodiments, chiral control of linkage phosphorus of chiral internucleotidic linkages can be utilized in oligonucleotides to provide various properties and/or activities. In some embodiments, a Rp internucleotidic linkage (e.g., a Rp phosphorothioate internucleotidic linkage), a Sp internucleotidic linkage (e.g., a Rp phosphorothioate internucleotidic linkage), or a non-chirally controlled internucleotidic linkage (e.g., a non-chirally controlled phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine (“+” is counting from the nucleoside toward the 5′-end of an oligonucleotide with the internucleotidic linkage at the +1 position being the internucleotidic linkage between a nucleoside opposite to a target adenosine and its 5′ side neighboring nucleoside (e.g., being the internucleotidic linkage bonded to the 5′-carbon of a nucleoside opposite to a target adenosine, or being between N₁ and N₀ of 5′-N₁N₀N₋₁-3′, wherein as described herein N₀ is the nucleoside opposite to a target adenosine), and “−” is counting from the nucleoside toward the 3′-end of an oligonucleotide with the internucleotidic linkage at the −1 position being the internucleotidic linkage between a nucleoside opposite to a target adenosine and its 3′ side neighboring nucleoside (e.g., being the internucleotidic linkage bonded to the 3′-carbon of a nucleoside opposite to a target adenosine, or being between N₋₁ and No of 5′-N₁N₀N₋₁-3′, wherein as described herein N₀ is the nucleoside opposite to a target adenosine)). In some embodiments, a Rp internucleotidic linkage (e.g., a Rp phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine. In some embodiments, a Rp internucleotidic linkage (e.g., a Rp phosphorothioate internucleotidic linkage) is at one or more of positions −2, −1, +3, +4, +5, +6, +7, and +8 of a nucleoside opposite to a target adenosine. In some embodiments, a Sp internucleotidic linkage (e.g., a Sp phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine. In some embodiments, a Sp internucleotidic linkage (e.g., a Sp phosphorothioate internucleotidic linkage) is at one or more of positions −2, −1, +3, +4, +5, +6, +7, and +8 of a nucleoside opposite to a target adenosine. In some embodiments, a non-chirally controlled internucleotidic linkage (e.g., a non-chirally controlled phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine. In some embodiments, a non-chirally controlled internucleotidic linkage (e.g.. a non-chirally controlled phosphorothioate internucleotidic linkage) is at one or more of positions −2, −1, +3, +4, +5, +6, +7, and +8 of a nucleoside opposite to a target adenosine.

In some embodiments, Rp is at position +8. In some embodiments, Rp is at position +7. In some embodiments, Rp is at position −6. In some embodiments, Rp is at position +5. In some embodiments, Rp is at position +4. In some embodiments, Rp is at position +3. In some embodiments, Rp is at position +2. In some embodiments, Rp is at position +1. In some embodiments, Rp is at position −1. In some embodiments, Rp is at position −2. In some embodiments, Rp is at position −3. In some embodiments, Rp is at position −4. In some embodiments, Rp is at position −5. In some embodiments, Rp is at position −6. In some embodiments, Rp is at position −7. In some embodiments, Rp is at position −8. In some embodiments, Rp is the configuration of a chirally controlled phosphorothioate internucleotidic linkage. In some embodiments, Sp is at position +8. In some embodiments, Sp is at position +7. In some embodiments, Sp is at position −6. In some embodiments, Sp is at position +5. In some embodiments, Sp is at position +4. In some embodiments, Sp is at position +3. In some embodiments, Sp is at position +2. In some embodiments, Sp is at position +1. In some embodiments, Sp is at position −1. In some embodiments, Sp is at position −2. In some embodiments, Sp is at position −3. In some embodiments, Sp is at position −4. In some embodiments, Sp is at position −5. In some embodiments, Sp is at position −6. In some embodiments, Sp is at position −7. In some embodiments, Sp is at position −8. In some embodiments, Sp is the configuration of a chirally controlled phosphorothioate internucleotidic linkage. In some embodiments, a non-chirally controlled internucleotidic linkage is at position +8. In some embodiments, a non-chirally controlled internucleotidic linkage is at position +7. In some embodiments, a non-chirally controlled internucleotidic linkage is at position -6. In some embodiments, a non-chirally controlled internucleotidic linkage is at position +5. In some embodiments, a non-chirally controlled internucleotidic linkage is at position +4. In some embodiments, a non-chirally controlled internucleotidic linkage is at position +3. In some embodiments, a non-chirally controlled internucleotidic linkage is at position +2. In some embodiments, a non-chirally controlled internucleotidic linkage is at position +1. In some embodiments, a non-chirally controlled internucleotidic linkage is at position −1. In some embodiments, a non-chirally controlled internucleotidic linkage is at position −2. In some embodiments, a non-chirally controlled internucleotidic linkage is at position −3. In some embodiments, a non-chirally controlled internucleotidic linkage is at position −4. In some embodiments, a non-chirally controlled internucleotidic linkage is at position −5. In some embodiments, a non-chirally controlled internucleotidic linkage is at position −6. In some embodiments, a non-chirally controlled internucleotidic linkage is at position −7. In some embodiments, a non-chirally controlled internucleotidic linkage is at position −8. In some embodiments, a non-chirally controlled internucleotidic linkage is a non-chirally controlled phosphorothioate internucleotidic linkage.

In some embodiments, a first domain comprises one or more (e.g., 1, 2, 3, 4, 5, 6, 7, 8, 9, or 10) Rp internucleotidic linkages (e.g., Rp phosphorothioate internucleotidic linkages). In some embodiments, a first domain comprises one or more (e.g., 1, 2, 3, 4, 5, 6, 7, 8, 9, or 10) Sp internucleotidic linkages (e.g., Sp phosphorothioate internucleotidic linkages). In some embodiments, a first domain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, at least about 50%, 55%, 60%, 65%, 70%, 75%, 80%, 85%, 90%, or 95%, or all of internucleotidic linkages in a first domain are chirally controlled and are Sp. In some embodiments, at least about 50%, 55%, 60%, 65%, 70%, 75%, 80%, 85%, 90%, or 95%, or all of phosphorothioate internucleotidic linkages in a first domain are chirally controlled and are Sp. In some embodiments, a second domain comprises one or more (e.g., 1, 2, 3, 4, 5, 6, 7, 8, 9, or 10) Rp internucleotidic linkages (e.g., Rp phosphorothioate internucleotidic linkages). In some embodiments, a second domain comprises one or more (e.g., 1, 2, 3, 4, 5, 6, 7, 8, 9, or 10) Sp internucleotidic linkages (e.g., Sp phosphorothioate internucleotidic linkages). In some embodiments, a second domain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, at least about 50%, 55%, 60%, 65%, 70%, 75%, 80%, 85%, 90%, or 95%, or all of internucleotidic linkages in a second domain are chirally controlled and are Sp. In some embodiments, at least about 50%, 55%, 60%, 65%, 70%, 75%, 80%, 85%, 90%, or 95%, or all of phosphorothioate internucleotidic linkages in a second domain are chirally controlled and are Sp. In some embodiments, a first subdomain comprises one or more (e.g., 1, 2, 3, 4, 5, 6, 7, 8, 9, or 10) Rp internucleotidic linkages (e.g., Rp phosphorothioate internucleotidic linkages). In some embodiments, a first subdomain comprises one or more (e.g., 1, 2, 3, 4, 5, 6, 7, 8, 9, or 10) Sp internucleotidic linkages (e.g., Sp phosphorothioate internucleotidic linkages). In some embodiments, a first subdomain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, such internucleotidic linkages are at 3′-end portion of a first subdomain.

In some embodiments, one or more natural phosphate linkages are utilized in provided oligonucleotides and compositions thereof. In some embodiments, provided oligonucleotides or portions thereof (e.g., first domains, second domains, first subdomains, second subdomains, third subdomains, etc.) comprise one or more (e.g., about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, or more) natural phosphate linkages. In some embodiments, provided oligonucleotides or portions thereof (e.g., first domains, second domains, first subdomains, second subdomains, third subdomains, etc.) comprise two or more (e.g., about, or at least about, 2,3,4,5, 6,7, 8,9, 10, 11, 12, 13, 14, 15, 16, 17, 18, 19, 20, 21, 22, 23, 24, 25, 26, 27, 28, 29, 30, 35, 40, 45, or 50, or more) consecutive natural phosphate linkages. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages. In some embodiments, about or at least about 5%, 10%1, 15%, 20%, 25%, 30%, 35%, 40%,45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are natural phosphate linkages. In some embodiments, about or at least about 5%, 10%, 15%, 20%, 25%, 30%, 35%, 40%, 45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are not natural phosphate linkages. In some embodiments, about or at least about 5%, 10%, 15%, 20%, 25%, 30%, 35%, 40%, 45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are not consecutive natural phosphate linkages.

In some embodiments, provided oligonucleotides or portions thereof comprises one or more natural phosphate linkages and one or more modified internucleotidic linkages. In some embodiments, provided oligonucleotides or portions thereof comprises one or more natural phosphate linkages and one or more chirally controlled modified internucleotidic linkages. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages each of which independently bonds to two sugars comprising no 2′-OR modification, wherein R is as described herein but not —H. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages each of which independently bonds to two sugars comprising no 2′-OR modification, wherein R is as described herein but not —H. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages each of which independently bonds to two 2′-F modified sugars. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages each of which independently bonds to two 2′-F modified sugars. In some embodiments, in oligonucleotides or portions thereof (e.g., first domains, second domains, first subdomains, second subdomains, third subdomains, etc.) no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., internucleotidic linkages that bond to two 2′-F modified sugars are natural phosphate linkages. In some embodiments, in oligonucleotides or portions thereof (e.g., first domains, second domains, first subdomains, second subdomains, third subdomains, etc.) no more than about 5%, 10%, 15%, 20%, 25%, 30%, 35%,40%,45%, 50%, 55%, 60%, 65%,70%,75%, 80%, 85%, 90%, or 95%, e.g., no more than 10%, no more than 15%, no more than 20%, no more than 25%, no more than about 30%, no more than about 40%, no more than 50% etc., of internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 5%, 10%, 15%, 20%, 25%, 30%, 35%, 40%, 45%, 50%, 55%, 60%, 65%, 70%, 75%, 80%, 85%, 90%, or 95%, e.g., no more than 10%, no more than 15%, no more than 20%, no more than 25%, no more than about 30%, no more than about 40%, no more than 50% etc., of internucleotidic linkages that bond to two 2′-F modified sugars are natural phosphate linkages. In some embodiments, in oligonucleotides or portions thereof (e.g., first domains, second domains, first subdomains, second subdomains, third subdomains, etc.) no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., consecutive internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., consecutive internucleotidic linkages that bond to two 2′-F modified sugars are natural phosphate linkages.

In some embodiments, a natural phosphate linkage is at one or more of positions −8, −7, −6, −5, −4, −3, −2, −1, +1, +2, +3, +4, +5, +6, +7, and +8 of a nucleoside opposite to a target adenosine. In some embodiments, a natural phosphate linkage is at one or more of positions −1 and +1. In some embodiments, a natural phosphate linkage is at positions −1 and +1. In some embodiments, a natural phosphate linkage is at position −1. In some embodiments, a natural phosphate linkage is at position +1. In some embodiments, a natural phosphate linkage is at position +8. In some embodiments, a natural phosphate linkage is at position +7. In some embodiments, a natural phosphate linkage is at position −6. In some embodiments, a natural phosphate linkage is at position +5. In some embodiments, a natural phosphate linkage is at position +4. In some embodiments, a natural phosphate linkage is at position +3. In some embodiments, a natural phosphate linkage is at position +2. In some embodiments, a natural phosphate linkage is at position −2. In some embodiments, a natural phosphate linkage is at position −3. In some embodiments, a natural phosphate linkage is at position −4. In some embodiments, a natural phosphate linkage is at position −5. In some embodiments, a natural phosphate linkage is at position −6. In some embodiments, a natural phosphate linkage is at position −7. In some embodiments, a natural phosphate linkage is at position −8. In some embodiments, a natural phosphate linkage is at position −1, and a modified internucleotidic linkage is at position +1. In some embodiments, a natural phosphate linkage is at position +1, and a modified internucleotidic linkage is at position −1. In some embodiments, a modified internucleotidic linkage is chirally controlled. In some embodiments, a modified internucleotidic linkage is chirally controlled and is Sp. In some embodiments, a modified internucleotidic linkage is a chirally controlled Sp phosphorothioate internucleotidic linkage. In some embodiments, a modified internucleotidic linkage is chirally controlled and is Rp. In some embodiments, a modified internucleotidic linkage is a chirally controlled Rp phosphorothioate internucleotidic linkage. In some embodiments, a second domain comprises no more than 2 natural phosphate linkages. In some embodiments, a second domain comprises no more than 1 natural phosphate linkages. In some embodiments, a single natural phosphate linkage can be utilized at various positions of an oligonucleotide or a portion thereof (e.g., a first domain, a second domain, a first subdomain, a second subdomain, a third subdomain, etc.).

In some embodiments, particular types of sugars are utilized at particular positions of oligonucleotides or portions thereof. For example, in some embodiments, a first domain comprises a number of 2′-F modified sugars (and optionally a number of 2′-OR modified sugars wherein R is not-H, in some embodiments at lower levels than 2′-F modified sugars), a first subdomain comprises a number of 2′-OR modified sugars wherein R is not-H (e.g., 2′-OMe modified sugars; and optionally a number of 2′-F sugars, in some embodiments at lower levels than 2′-OR modified sugars wherein R is not —H), a second domain comprises one or more natural DNA sugars (no substitution at 2′ position) and/or one or more 2′-F modified sugars, and/or a third subdomain comprises a number of 2′-OR modified sugars wherein R is not-H (e.g., 2′-OMe modified sugars; and optionally a number of 2′-F sugars, in some embodiments at lower levels than 2′-OR modified sugars wherein R is not —H). In some embodiments, particular type of sugars are independently at one or more of positions −8, -7, -6, -5, -4, -3, -2, -1, 0, +1, +2, +3, +4, +5, +6, +7, and +8 of a nucleoside opposite to a target adenosine (“+” is counting from the nucleoside toward the 5′-end of an oligonucleotide, “-“is counting from the nucleoside toward the 3′-end of an oligonucleotide, with position 0 being the position of the nucleoside opposite to a target adenosine, e.g.: 5′- . . . N₊₂N₊₁N₀N₁N₋₂ . . . 3′). In some embodiments, particular types of sugars are independently at one or more of positions −5, −4, −3, −2, −1, 0, +1, +2, +3, +4, and +5. In some embodiments, particular types of sugars are independently at one or more of positions −3, −2, −1, 0, +1, +2, and +3. In some embodiments, particular types of sugars are independently at one or more of positions −2, −1, 0, +1, and +2. In some embodiments, particular types of sugars are independently at one or more of positions −1, 0, and +1. In some embodiments, a particular type of sugar is at position +8. In some embodiments, a particular type of sugar is at position +7. In some embodiments, a particular type of sugar is at position +6. In some embodiments, a particular type of sugar is at position +5. In some embodiments, a particular type of sugar is at position +4. In some embodiments, a particular type of sugar is at position +3. In some embodiments, a particular type of sugar is at position +2. In some embodiments, a particular type of sugar is at position +1. In some embodiments, a particular type of sugar is at position 0. In some embodiments, a particular type of sugar is at position −8. In some embodiments, a particular type of sugar is at position −7. In some embodiments, a particular type of sugar is at position −6. In some embodiments, a particular type of sugar is at position −5. In some embodiments, a particular type of sugar is at position −4. In some embodiments, a particular type of sugar is at position −3. In some embodiments, a particular type of sugar is at position −2. In some embodiments, a particular type of sugar is at position −1. In some embodiments, a particular type of sugar is independently a sugar selected from a natural DNA sugar (two 2′-H at 2′-carbon), a 2′-OMe modified sugar, and a 2′-F modified sugar. In some embodiments, a particular type of sugar is independently a sugar selected from a natural DNA sugar (two 2′-H at 2′-carbon) and a 2′-OMe modified sugar. In some embodiments, a particular type of sugar is independently a sugar selected from a natural DNA sugar (two 2′-H at 2′-carbon) and a 2′-F modified sugar, e.g., for sugars at position 0, −1, and/or +1. In some embodiments, a particularly type of sugar is a natural DNA sugar (two 2′-H at 2′-carbon), e.g., at position −1, 0 or +1. In some embodiments, a particular type of sugar is 2′-F modified sugar, e.g., at position −8, −7, −6, −5, −4, −3, −2, −1, 0, +1, +2, +3, +4, +5, +6, +7, and/or +8. In some embodiments, a particular type of sugar is 2′-F modified sugar, e.g., at position −8, −7, −6, −5, −4, −3, −2, +2, +3, +4, +5, +6, +7, and/or +8. In some embodiments, a 2′-F modified sugar is at position −2. In some embodiments, a 2′-F modified sugar is at position −3. In some embodiments, a 2′-F modified sugar is at position −4. In some embodiments, a 2′-F modified sugar is at position +2. In some embodiments, a 2′-F modified sugar is at position +3. In some embodiments, a 2′-F modified sugar is at position +4. In some embodiments, a 2′-F modified sugar is at position +5. In some embodiments, a 2′-F modified sugar is at position +6. In some embodiments, a 2′-F modified sugar is at position +7. In some embodiments, a 2′-F modified sugar is at position +8. In some embodiments, a particular type of sugar is 2′-OMe modified sugar, e.g., at position −8, −7, −6, −5, −4, −3, −2, −1, 0, +1, +2, +3, +4, +5, +6, +7, and/or +8. In some embodiments, a particular type of sugar is 2′-OMe modified sugar, e.g., at position −8, −7, −6, −5, −4, −3, −2, +2, +3, +4, +5, +6, +7, and/or +8. In some embodiments, a 2′-OMe modified sugar is at position -2. In some embodiments, a 2′-OMe modified sugar is at position −3. In some embodiments, a 2′-OMe modified sugar is at position −4. In some embodiments, a 2′-OMe modified sugar is at position +2. In some embodiments, a 2′-OMe modified sugar is at position +3. In some embodiments, a 2′-OMe modified sugar is at position +4. In some embodiments, a 2′-OMe modified sugar is at position +5. In some embodiments, a 2′-OMe modified sugar is at position +6. In some embodiments, a 2′-OMe modified sugar is at position +7. In some embodiments, a 2′-OMe modified sugar is at position +8. In some embodiments, a sugar at position 0 is not a 2′-MOE modified sugar. In some embodiments, a sugar at position 0 is a natural DNA sugar (two 2′-H at 2′-carbon). In some embodiments, a sugar at position 0 is not a 2′-MOE modified sugar. In some embodiments, a sugar at position −1 is not a 2′-MOE modified sugar. In some embodiments, a sugar at position −2 is not a 2′-MOE modified sugar. In some embodiments, a sugar at position −3 is not a 2′-MOE modified sugar. In some embodiments, a first domain comprises one or more 2′-F modified sugars, and optionally 2′-OR modified sugars (in some embodiments at lower levels than 2′-F modified sugars) wherein R is as described herein and is not —H. In some embodiments, a first domain comprises 1, 2, 3, 4, 5, 6, 7, 8, 9, or 10 2′—OR modified sugars (in some embodiments at lower levels that 2′-F modified sugars) wherein R is as described herein and is not —H. In some embodiments, a first domain comprise 1, 2, 3, or 4, or 1 and no more than 1, 2 and no more than 2, 3 and no more than 3, or 4 and no more than 4 2′-OR modified sugars wherein R is C₁₋₆ aliphatic. In some embodiments, the first, second, third and/or fourth sugars of a first domain are independently 2′-OR modified sugars, wherein R is optionally substituted C₁₋₆ aliphatic. In some embodiments, sugars comprising 2′-OR are consecutive. In some embodiments, a first domain comprises 2, 3, 4, 5, 6, 7, 8, 9, or 10 consecutive sugars at its 5′-end, wherein each sugar independently comprises 2′-OR, wherein R is optionally substituted C₁₋₆ aliphatic. In some embodiments, 2′-OR is 2′-OMe. In some embodiments, 2′-OR is 2′-MOE. In some embodiments, a second domain comprises one or more 2′-OR modified sugars (in some embodiments at lower levels) wherein R is as described herein and is not —H, and optionally 2′-F modified sugars (in some embodiments at lower levels). In some embodiments, a first subdomain comprises one or more 2′-OR modified sugars (in some embodiments at lower levels) wherein R is as described herein and is not —H, and optionally 2′-F modified sugars (in some embodiments at lower levels). In some embodiments, a third subdomain comprises one or more 2′-OR modified sugars (in some embodiments at lower levels) wherein R is as described herein and is not —H, and optionally 2′-F modified sugars (in some embodiments, at lower levels; in some embodiments, at higher levels). In some embodiments, a third subdomain comprises about, or at least about 1, 2, 3, 4, 5, 6, 7, 8, 9, or 10 2′-F modified sugars. In some embodiments, a third subdomain comprises about, or at least about 1, 2, 3, 4, 5, 6, 7, 8, 9, or 10 consecutive 2′-F modified sugars. In some embodiments, about or at least about, 50%, 55%, 60%, 65%, 70%, 75%, 80%, 85%, 90%, or 95% of sugars in a third subdomain independently comprise 2′-F modification. In some embodiments, the first 2′-F modified sugar in the third subdomain (from 5′ to 3′) is not the first sugar in the third subdomain. In some embodiments, the first 2′-F modified sugar in the third subdomain is at position -3 relative to the nucleoside opposition to a target adenosine. In some embodiments, each sugar in a third subdomain is independently a modified sugar. In some embodiments, each sugar in a third subdomain is independently a modified sugar, wherein the modification is selected from 2′-F and 2′-OR, wherein R is C₁₋₆ aliphatic. In some embodiments, a modification in selected from 2′-F and 2′-OMe. In some embodiments, each modified sugar in a third subdomain is independently 2′-F modified sugar. In some embodiments, each modified sugar in a third subdomain is independently 2′-OMe modified sugar. In some embodiments, one or more modified sugars in a third subdomain are independently 2′-OMe modified sugar, and one or more modified sugars in a third subdomain are independently 2′-F modified sugar. In some embodiments, each modified sugar in a third subdomain is independently a 2′-F modified sugar except the first sugar of a third subdomain, which in some embodiments is a 2′-OMe modified sugar. In some embodiments, a third subdomain comprises one or more 2′-OR modified sugars (in some embodiments at lower levels) wherein R is as described herein and is not —H, and optionally 2′-F modified sugars (in some embodiments at lower levels). In some embodiments, 2′-OR is 2′-OMe. In some embodiments, 2′-OR is 2′-MOE.

### Editing Region

In some embodiments, the present disclosure provides oligonucleotides comprising editing regions, e.g., regions comprising or consisting of 5′-N₁N₀N₋₁-3′ as described herein. In some embodiments, an editing region is or comprises a nucleoside opposite to a target adenosine (typically, when base sequences of oligonucleotides are aligned with target sequences for maximal complementarity, and/or oligonucleotides hybridize with target nucleic acids) and its neighboring nucleosides. In some embodiments, an editing region is or comprises three nucleobases, wherein the nucleobase in the middle is a nucleoside opposite to a target adenosine. In some embodiments, a nucleoside opposite to a target adenosine is No as described herein. In some embodiments, an editing region is or comprises a second subdomain. In some embodiments, a second subdomain is or comprising an editing region. In some embodiments, editing regions comprises three and no more than three nucleosides, the middle one of which is opposite to a target adenosine when utilized for editing, and such editing regions may be referred to as central triplets in various reports. Certain useful sugars, nucleobases, nucleosides, internucleotidic linkages etc. for editing regions are described in WO 2016/097212, WO 2017/050306, WO 2017/220751, WO 2018/041973, WO 2018/134301, WO 2019/158475, WO 2019/219581, WO 2020/154342, WO 2020/154343, WO 2020/154344, WO 2020/157008, WO 2020/165077, WO 2020/201406, WO 2020/216637, WO 2020/252376, WO 2021/130313, WO 2021/231673, WO 2021/231675, WO 2021/231679, WO 2021/231680, WO 2021/231685, WO 2021/231691, WO 2021/231692, WO 2021/231698, WO 2021/231830, WO 2021/243023, WO 2022/018207, or WO 2022/026928 and can be utilized in accordance with the present disclosure. Certain useful sugars, nucleobases, nucleosides, internucleotidic linkages etc. for editing regions are described in WO 2021/071858, WO 2022/099159, and/or PCT/US2023/018742 and are incorporated herein by reference.

In some embodiments, the nucleobase of a nucleoside opposite to a target adenosine (e.g., in some embodiments, N₀) (may be referred to as BA₀) is C. In some embodiments, BA₀ is a modified nucleobase as described herein. In some embodiments, a nucleobase, e.g., BA₀. is or comprises Ring BA which has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a nucleobase is optionally substituted or protected, or optionally substituted or protected tautomer of C, T, U, hypoxanthine, b001U, b002U, b003U, b004U, b005U, b006U, b007U, b008U, b009U, b 011U, b012U, b013U, b014U, b015U, b001A, b002A, b003A, b004A, b005A, b006A, b007A, b001G, b002G, b001C, b002C, b003C, b004C, b005C, b006C. b007C, b008C, b009C, b0021, b0031, b0041, b014I, [3nT], [3ne5U], [3nfl5U], [3npry5U], [3ncn5U], [nathp6o8A], [ipr6o8A], [c7In], [c39z481n], [z2c3In], [z5C], and zdnp. In some embodiments, a nucleobase is optionally substituted or protected, or optionally substituted or protected tautomer of zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b0021, b0031, or b001G. In some embodiments, the nucleobase of N₀ is optionally substituted or protected, or optionally substituted or protected tautomer of C, zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b002I, b0031, or b001G, and the sugar of N₀ is a natural DNA sugar. In some embodiments, the nucleobase of N₀ is optionally substituted or protected, or optionally substituted or protected tautomer of C, zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b002I, b003I, or b001G, and the sugar of N₀ is a natural RNA sugar. In some embodiments, the nucleobase of N₀ is optionally substituted or protected, or optionally substituted or protected tautomer of C, zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b002I, b003I, or b001G, and the sugar of N₀ is a 2′-F modified sugar. In some embodiments, the nucleobase of N₀ is optionally substituted or protected, or optionally substituted or protected tautomer of C, zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b002I, b003I, or b001G, and the sugar of N₀ is a bicyclic sugar (e.g., a LNA sugar, a cEt sugar, etc.). In some embodiments, the nucleobase of N₀ is optionally substituted or protected, or optionally substituted or protected tautomer of C, zdnp, b001U, b002U, b003U, b004U, b005U, b006U, b007U, b008U, b009U, b011U, b012U, b013U, b014U, b015U, b001A, b002A, b003A, b004A, b005A, b006A, b007A, b001C, b002C, b003C, b0021, b003I, or b001G, and the sugar of N₀ is a sugar comprising a 6-membered ring, e.g., sm19. In some embodiments, BA₀ is C. In some embodiments, BA₀ is T. In some embodiments, BA₀ is hypoxanthine. In some embodiments, BA₀ is U. In some embodiments, BA₀ is b001U. In some embodiments, BA₀ is b002U. In some embodiments, BA₀ is b003U. In some embodiments, BA₀ is b004U. In some embodiments, BA₀ is b005U. In some embodiments, BA₀ is b006U. In some embodiments, BA₀ is b007U. In some embodiments, BA₀ is b008U. In some embodiments, BA₀ is b009U. In some embodiments, BA₀ is b011U. In some embodiments, BA₀ is b012U. In some embodiments, BA₀ is b013U. In some embodiments, BA₀ is b014U. In some embodiments, BA₀ is b015U. In some embodiments, BA₀ is b001A. In some embodiments, BA₀ is b002A. In some embodiments, BA₀ is b003A. In some embodiments, BA₀ is b004A. In some embodiments, BA₀ is b005A. In some embodiments, BA₀ is b006A. In some embodiments, BA₀ is b007A. In some embodiments, BA₀ is b001C. In some embodiments, BA₀ is b002C. In some embodiments, BA₀ is b003C. In some embodiments, BA₀ is b004C. In some embodiments, BA₀ is b005C. In some embodiments, BA₀ is b006C. In some embodiments, BA₀ is b007C. In some embodiments, BA₀ is b008C. In some embodiments, BA₀ is b009C. In some embodiments, BA₀ is b002I. In some embodiments, BA₀ is b003I. In some embodiments, BA₀ is b004L. In some embodiments, BA₀ is b014I. In some embodiments, BA₀ is b001G. In some embodiments, BA₀ is b002G. In some embodiments, BA₀ is [3nT]. In some embodiments, BA₀ is [3ne5U]. In some embodiments, BA₀ is [3nflU]. In some embodiments, BA₀ is [3npry5U]. In some embodiments, BA₀ is [3ncn5U]. In some embodiments, BA₀ is [nathp6o8A]. In some embodiments, BA₀ is [ipr6o8A]. In some embodiments, BA₀ is [c7In]. In some embodiments, BA₀ is [c39z48In]. In some embodiments, BA₀ is [z2c3In]. In some embodiments, BA₀ is [z5C]. In some embodiments, BA₀ is C. In some embodiments, BA₀ does not base pair with A. In some embodiments, BA₀ base pairs with guanine (G) or inosine (I). In some embodiments, sugar of N₀ is a natural DNA sugar, or a substituted natural DNA sugar one of whose 2′-H is substituted with —OH or —F and the other 2′-H is not substituted. In some embodiments, sugar of N₀ is a natural DNA sugar. In some embodiments, sugar of N₀ is a natural RNA sugar. In some embodiments, sugar of N₀ is an acyclic sugar. In some embodiments, sugar of N₀ is sm01. In some embodiments, sugar of N₀ is sm04. In some embodiments, sugar of N₀ is sm11. In some embodiments, sugar of N₀ is sm12. In some embodiments, sugar of N₀ is rsm13. In some embodiments, sugar of N₀ is rsm14. In some embodiments, sugar of N₀ is sm15. In some embodiments, sugar of N₀ is sm16. In some embodiments, sugar of N₀ is sm17. In some embodiments, sugar of N₀ is sm18. In some embodiments, sugar of N₀ is sm19. In some embodiments, sugar of N₀ is a FANA sugar. In some embodiments, sugar of N₀ is a bicyclic sugar. In some embodiments, sugar of N₀ is a LNA sugar. In some embodiments, sugar of N₀ is a cEt sugar. In some embodiments, sugar of N₀ is a (S)-cEt sugar. For example, while various reports asserted that 2′-OMe modified sugar at N₀ blocked editing, various base modifications described herein, e.g., those of formula BA-III-e such as b008U, can be utilized with 2′-OMe modified sugar at N₀ to provide editing, in some cases with at least comparable levels compared to other sugars such as natural DNA sugar. In some embodiments, one or two or three sugars at N₋₁, No or N₋₁ are independently modified sugars. In some embodiments, a sugar at N₁ is a modified sugar. In some embodiments, a sugar at No is a modified sugar. In some embodiments, a sugar at N₋₁ is a modified sugar. In some embodiments, sugars at N₁ and No are each independently a modified sugar. In some embodiments, sugars at N₋₁ and No are each independently a modified sugar. In some embodiments, sugars at N₁ and N₋₁ are each independently a modified sugar. In some embodiments, sugars at N₁, No and N₋₁ are each independently a modified sugar. In some embodiments, a modified sugar is a 2′-modified sugar as described herein. In some embodiments, a modified sugar is a 2′-OR” modified sugar. In some embodiments, a modified sugar is a 2′-OMe modified sugar. In some embodiments, a modified sugar is a 2′-MOE modified sugar. In some embodiments, a modified sugar is a bicyclic sugar, e.g., a LNA sugar. Among other things, the present disclosure confirmed that various modified nucleobases and/or various sugars may be utilized at N₀ in oligonucleotides to provide adenosine-editing activities. In some embodiments, b001A as BA₀ can provide improved adenosine editing efficiency compared to a reference nucleobase (e.g., under comparable conditions including in otherwise identical oligonucleotides, assessed in identical or comparable assays, etc.). In some embodiments, b008U as BA₀ can provide improved adenosine editing efficiency. In some embodiments, a reference nucleobase is U. In some embodiments, a reference nucleobase is T. In some embodiments, a reference nucleobase is C. In some embodiments, when sugar of No comprises certain 6-membered ring, e.g., when sugar of N₀ is sm19, editing activity may be improved over a reference sugar, e.g., a natural DNA sugar. In some embodiments, LNA sugars can be utilized in N₀ or neighboring nucleosides.

In some embodiments, an oligonucleotide comprises a nucleoside, e.g., N₀, the nucleobase of which is a modified nucleobase as described herein, and the sugar of which is a sugar as described herein. For example, in some embodiments, a nucleoside is b001A. In some embodiments, a nucleoside is b008U. In some embodiments, the nucleobase of a nucleoside is b001A and the sugar is a natural DNA sugar. In some embodiments, the nucleobase of a nucleoside is b001A and the sugar is a 2′-modified sugar. In some embodiments, the nucleobase of a nucleoside is b001A and the sugar is a 2′-F modified sugar. In some embodiments, the nucleobase of a nucleoside is b001A and the sugar is a 2′-OR modified sugar wherein R is optionally substituted C₁₋₆ aliphatic. In some embodiments, the nucleobase of a nucleoside is b001A and the sugar is a 2′-OR modified sugar wherein R is optionally substituted C₁₋₆ alkyl. In some embodiments, the nucleobase of a nucleoside is b001A and the sugar is a 2′-OMe modified sugar. In some embodiments, the nucleobase of a nucleoside is b001A and the sugar is a 2′-MOE modified sugar. In some embodiments, the nucleobase of a nucleoside is b008U and the sugar is a natural DNA sugar. In some embodiments, the nucleobase of a nucleoside is b008U and the sugar is a 2′-modified sugar. In some embodiments, the nucleobase of a nucleoside is b008U and the sugar is a 2′-F modified sugar. In some embodiments, the nucleobase of a nucleoside is b008U and the sugar is a 2′-OR modified sugar wherein R is optionally substituted C₁₋₆ aliphatic. In some embodiments, the nucleobase of a nucleoside is b008U and the sugar is a 2′-OR modified sugar wherein R is optionally substituted C₁₋₆ alkyl. In some embodiments, the nucleobase of a nucleoside is b008U and the sugar is a 2′-OMe modified sugar. In some embodiments, the nucleobase of a nucleoside is b008U and the sugar is a 2′-MOE modified sugar. In some embodiments, a nucleoside opposite to a target adenosine, e.g., N₀, is dC. In some embodiments, it is rC. In some embodiments, it is fC. In some embodiments, it is dT. In some embodiments, it is rT. In some embodiments, it is fT. In some embodiments, it is dU. In some embodiments, it is rU. In some embodiments, it is fU. In some embodiments, it is b001A (which when utilized for a nucleoside refers to

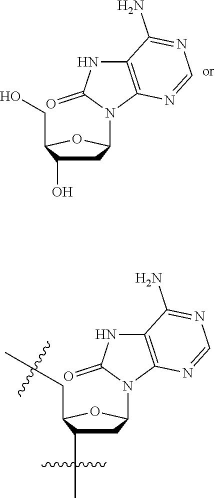

in an oligonucleotide chain unless specified otherwise). In some embodiments, it is Csm15 (which when utilized for a nucleoside refers to

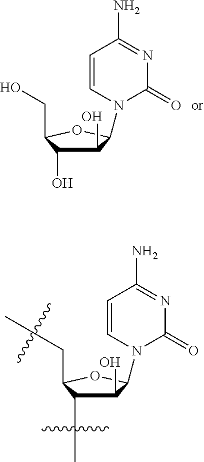

in an oligonucleotide chain unless specified otherwise). In some embodiments, it is Usm15 (which when utilized for a nucleoside refers to

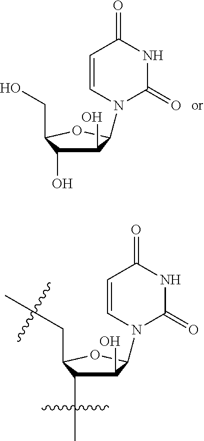

in an oligonucleotide chain unless specified otherwise). In some embodiments, it is rCsm13 (which when utilized for a nucleoside refers to

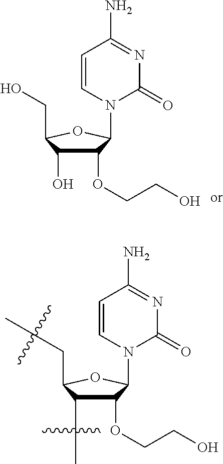

in an oligonucleotide chain unless specified otherwise). In some embodiments, it is Csm04 (which when utilized for a nucleoside refers to

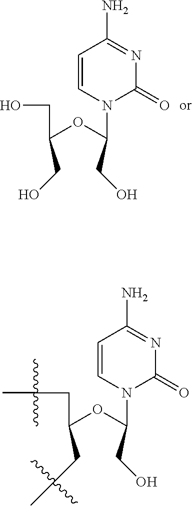

in an oligonucleotide chain unless specified otherwise). In some embodiments, it is b001rA (which when utilized for a nucleoside refers to

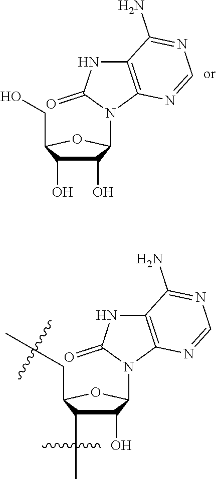

in an oligonucleotide chain unless specified otherwise). In some embodiments, a sugar is a (R)-GNA sugar

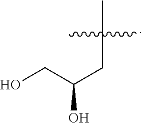 [SMILES: *C[C@@H](O)CO]

In some embodiments, a sugar is a (S)-GNA sugar

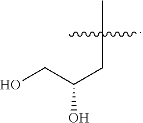 [SMILES: *C[C@H](O)CO]

In some embodiments, it is S-GNA C, also referred herein as

Csm 11 (which when utilized for a nucleoside refers to

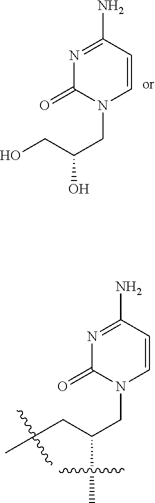

in an oligonucleotide chain unless specified otherwise). In some embodiments, it is R-GNA C, also referred herein as Csm12 (which when utilized for a nucleoside refers to

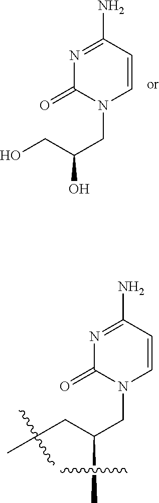

in an oligonucleotide chain unless specified otherwise). In some embodiments, it is S-GNA b008U, also referred herein as b008Usm11 (which when utilized for a nucleoside refers to

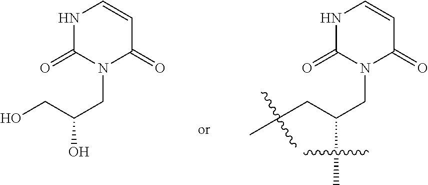

in an oligonucleotide chain unless specified otherwise). In some embodiments, it is R-GNA b008U, also referred herein as b008Usm12 (which when utilized for a nucleoside refers to

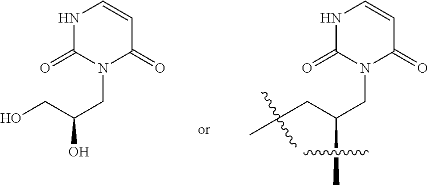

in an oligonucleotide chain unless specified otherwise). In some embodiments, it is S-GNA isoC, also referred herein as b009Csm11 (which when utilized for a nucleoside refers to

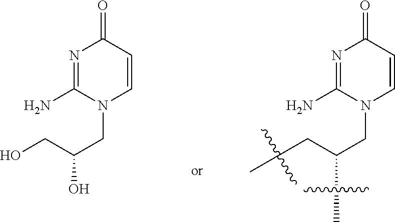

in an oligonucleotide chain unless specified otherwise). In some embodiments, it is R-GNA isoC, also referred herein as b009Csm12 (which when utilized for a nucleoside refers to

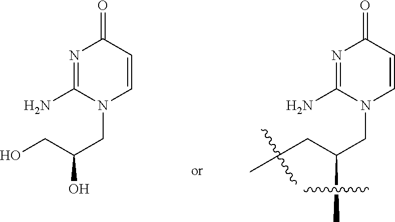

in an oligonucleotide chain unless specified otherwise). In some embodiments, it is S-GNA G, also referred herein as Gsm11 (which when utilized for a nucleoside refers to

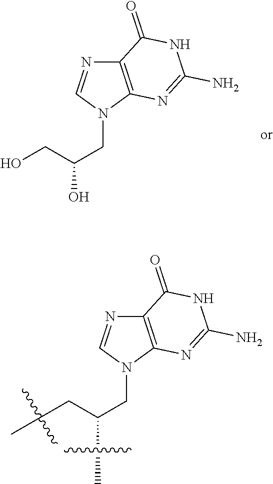

in an oligonucleotide chain unless specified otherwise). In some embodiments, it is R-GNA G, also referred herein as Gsm12 (which when utilized for a nucleoside refers to

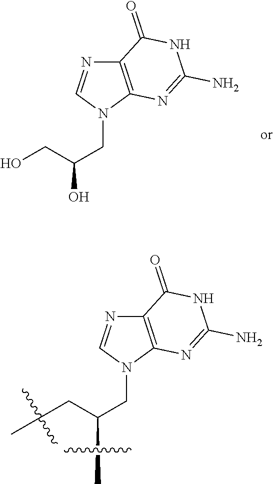

in an oligonucleotide chain unless specified otherwise). In some embodiments, it is S-GNA T. also referred herein as Tsm11 (which when utilized for a nucleoside refers to

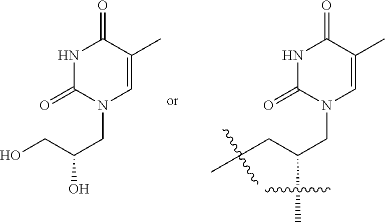

in an oligonucleotide chain unless specified otherwise). In some embodiments, it is R-GNA T, also referred herein as Tsm12 (which when utilized for a nucleoside refers to

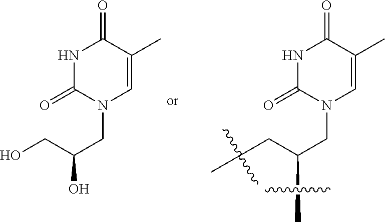

in an oligonucleotide chain unless specified otherwise). In some embodiments, it is b004C (which when utilized for a nucleoside refers to

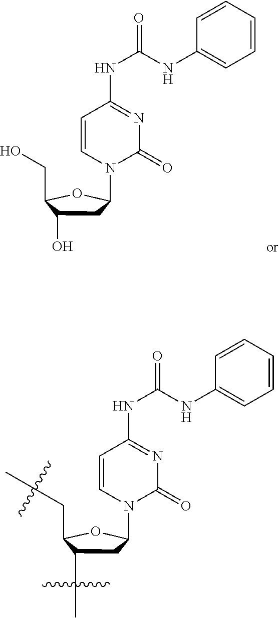

in an oligonucleotide chain unless specified otherwise). In some embodiments, it is b007C (which when utilized for a nucleoside refers to

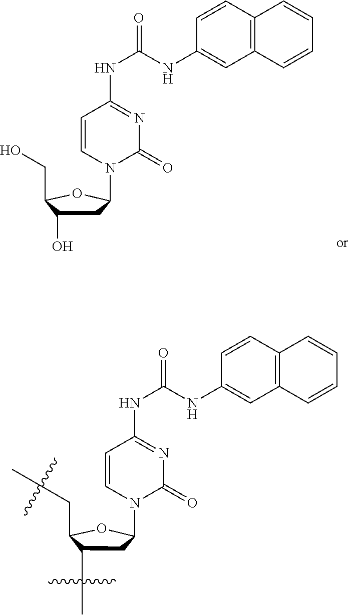

in an oligonucleotide chain unless specified otherwise).
In some embodiments, it is Csm16 (which when utilized for a nucleoside refers to

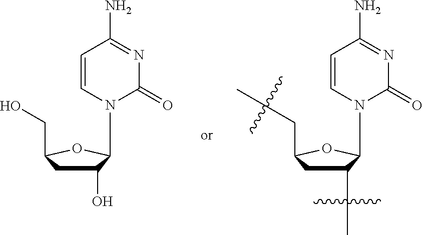 [SMILES: Nc1ccn([C@@H]2O[C@H](CO)C[C@@H]2O)c(=O)n1 ; *C[C@@H]1C[C@H](*)[C@H](n2ccc(N)nc2=O)O1]

in an oligonucleotide chain unless specified otherwise). In some embodiments, it is Csm17 (which when utilized for a nucleoside refers to

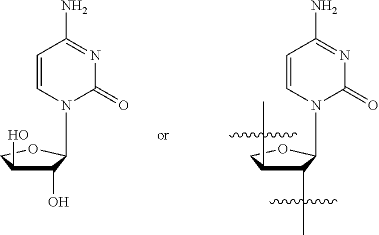

in an oligonucleotide chain unless specified otherwise). In some embodiments, it is rCsm14 (which when utilized for a nucleoside refers to

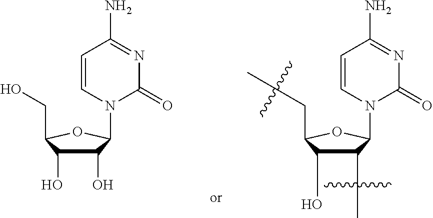

in an oligonucleotide chain unless specified otherwise). In some embodiments, it is b008U (which when utilized for a nucleoside refers to

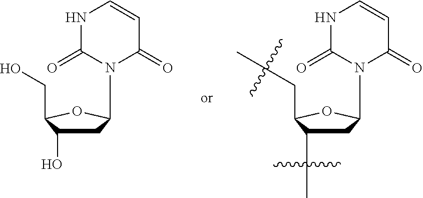

in an oligonucleotide chain unless specified otherwise). In some embodiments, it is fb008U (which when utilized for a nucleoside refers to

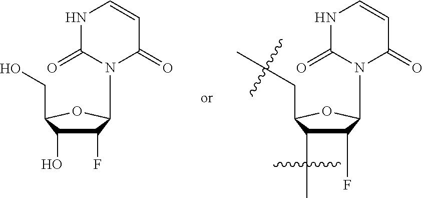

in an oligonucleotide chain unless specified otherwise). In some embodiments, it is mb008U (which when utilized for a nucleoside refers to

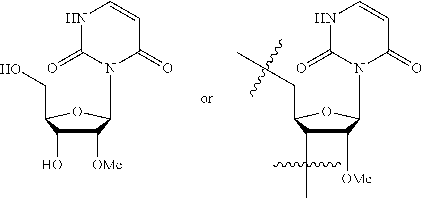

in an oligonucleotide chain unless specified otherwise). In some embodiments, it is b010U (which when utilized for a nucleoside refers to

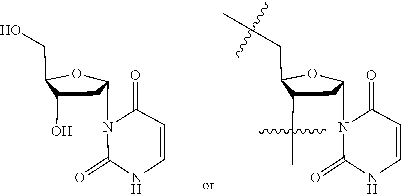 [SMILES: O=c1cc[nH]c(=O)n1[C@@H]1C[C@@H](O)[C@@H](CO)O1 ; *C[C@H]1O[C@H](n2c(=O)cc[nH]c2=O)C[C@H]1*]

in an oligonucleotide chain unless specified otherwise). In some embodiments, it is b001C (which when utilized for a nucleoside refers to

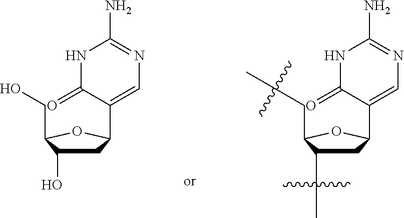

in an oligonucleotide chain unless specified otherwise). In some embodiments, it is b008C (which when utilized for a nucleoside refers to

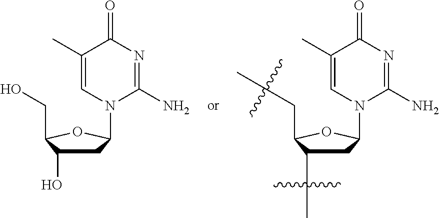

in an oligonucleotide chain unless specified otherwise). In some embodiments, it is b011U (which when utilized for a nucleoside refers to

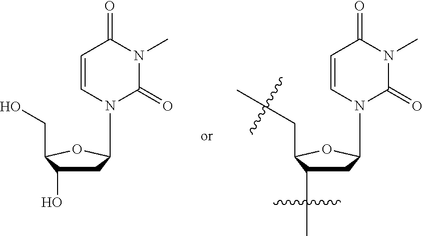

in an oligonucleotide chain unless specified otherwise). In some embodiments, it is b012U (which when utilized for a nucleoside refers to

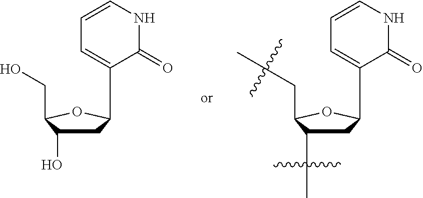

in an oligonucleotide chain unless specified otherwise). In some embodiments, it is abasic. In some embodiments, it is L010. In some embodiments, it is L034 (which when utilized for a nucleoside refers to

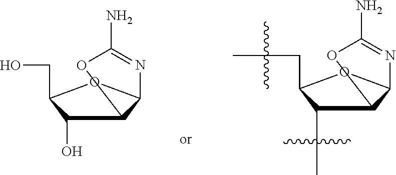

in an oligonucleotide chain unless specified otherwise). In some embodiments, it is b002G (which when utilized for a nucleoside refers to

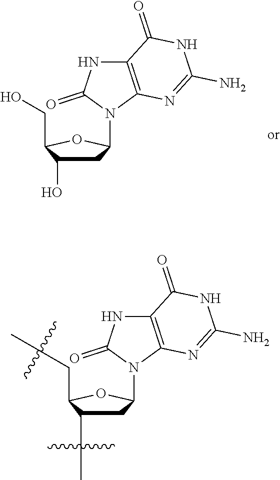

in an oligonucleotide chain unless specified otherwise). In some embodiments, it is b013U (which when utilized for a nucleoside refers to

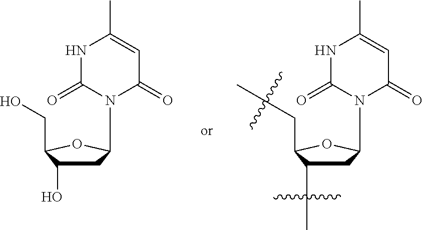

in an oligonucleotide chain unless specified otherwise). In some embodiments, it is b002A (which when utilized for a nucleoside refers to

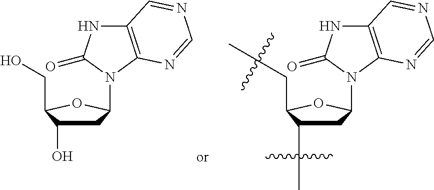

in an oligonucleotide chain unless specified otherwise). In some embodiments, it is b003A (which when utilized for a nucleoside refers to

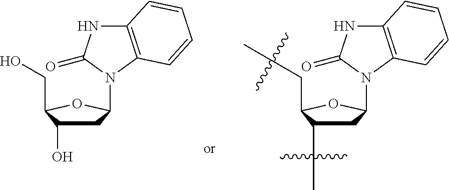

in an oligonucleotide chain unless specified otherwise). In some embodiments, it is b004I (which when utilized for a nucleoside refers to

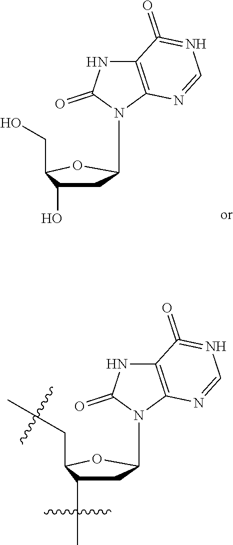

in an oligonucleotide chain unless specified otherwise). In some embodiments, it is b014I (which when utilized for a nucleoside refers to

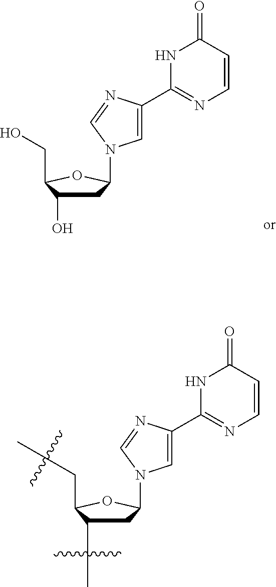

in an oligonucleotide chain unless specified otherwise). In some embodiments, it is b009U (which when utilized for a nucleoside refers to

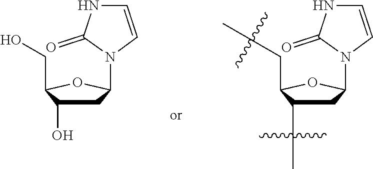

in an oligonucleotide chain unless specified otherwise). In some embodiments, it is aC (which when utilized for a nucleoside refers to

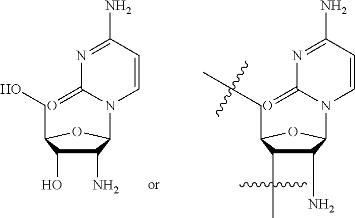

in an oligonucleotide chain unless specified otherwise). In some embodiments, it is b001U (which when utilized for a nucleoside refers to

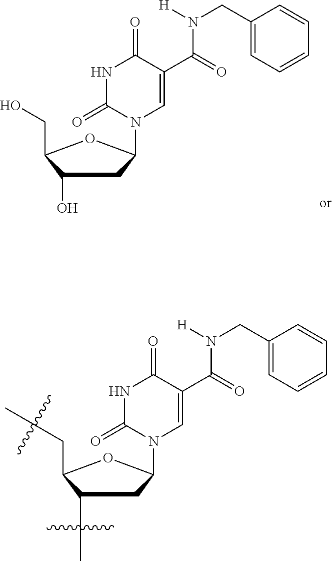 [SMILES: O=C(NCc1ccccc1)c1cn([C@H]2C[C@@H](O)[C@@H](CO)O2)c(=O)[nH]c1=O ; *C[C@H]1O[C@@H](n2cc(C(=O)NCc3ccccc3)c(=O)[nH]c2=O)C[C@H]1*]

in an oligonucleotide chain unless specified otherwise). In some embodiments, it is b002U (which when utilized for a nucleoside refers to

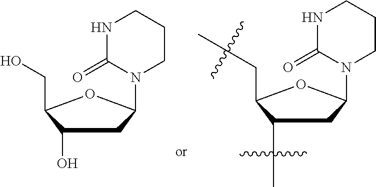 [SMILES: O=C1NCCCN1[C@H]1C[C@@H](O)[C@@H](CO)O1 ; *C[C@H]1O[C@@H](N2CCCNC2=O)C[C@H]1*]

in an oligonucleotide chain unless specified otherwise). In some embodiments, it is b003U (which when utilized for a nucleoside refers to

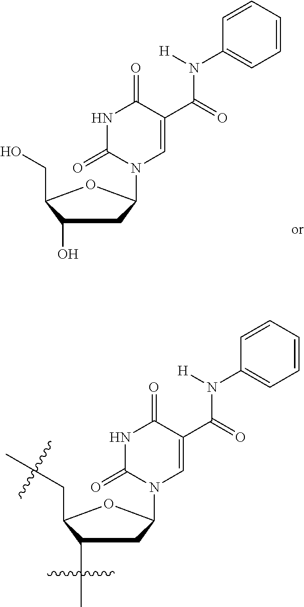 [SMILES: O=C(Nc1ccccc1)c1cn([C@H]2C[C@@H](O)[C@@H](CO)O2)c(=O)[nH]c1=O ; *C[C@H]1O[C@@H](n2cc(C(=O)Nc3ccccc3)c(=O)[nH]c2=O)C[C@H]1*]

in an oligonucleotide chain unless specified otherwise). In some embodiments, it is b004U (which when utilized for a nucleoside refers to

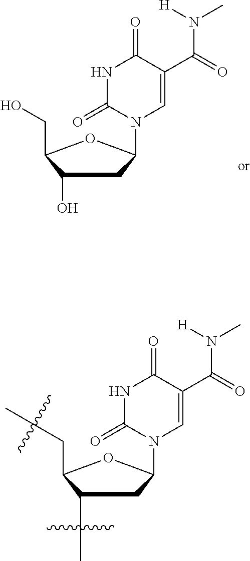 [SMILES: CNC(=O)c1cn([C@H]2C[C@@H](O)[C@@H](CO)O2)c(=O)[nH]c1=O ; *C[C@H]1O[C@@H](n2cc(C(=O)NC)c(=O)[nH]c2=O)C[C@H]1*]

in an oligonucleotide chain unless specified otherwise). In some embodiments, it is b005U (which when utilized for a nucleoside refers to

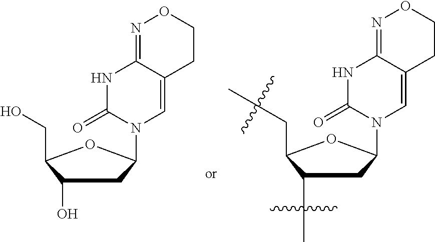 [SMILES: O=C1NC2=NOCCC2=CN1[C@H]1C[C@@H](O)[C@@H](CO)O1 ; *C[C@H]1O[C@@H](N2C=C3CCON=C3NC2=O)C[C@H]1*]

in an oligonucleotide chain unless specified otherwise). In some embodiments, it is b006U (which when utilized for a nucleoside refers to

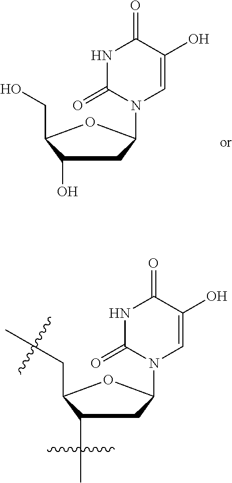 [SMILES: O=c1[nH]c(=O)n([C@H]2C[C@@H](O)[C@@H](CO)O2)cc1O ; *C[C@H]1O[C@@H](n2cc(O)c(=O)[nH]c2=O)C[C@H]1*]

in an oligonucleotide chain unless specified otherwise). In some embodiments, it is b007U (which when utilized for a nucleoside refers to

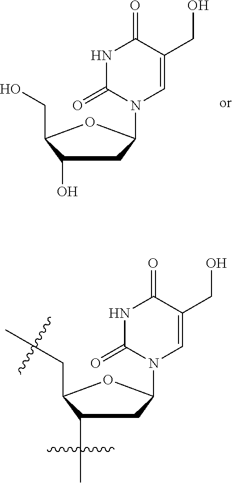 [SMILES: O=c1[nH]c(=O)n([C@H]2C[C@@H](O)[C@@H](CO)O2)cc1CO ; *C[C@H]1O[C@@H](n2cc(CO)c(=O)[nH]c2=O)C[C@H]1*]

in an oligonucleotide chain unless specified otherwise). In some embodiments, it is b001G (which when utilized for a nucleoside refers to

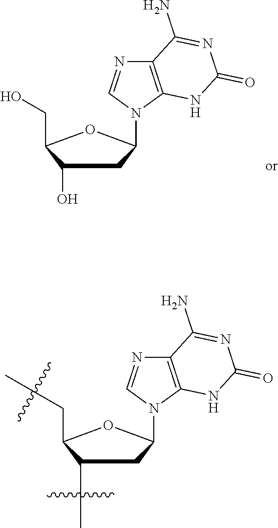 [SMILES: Nc1nc(=O)[nH]c2c1ncn2[C@H]1C[C@@H](O)[C@@H](CO)O1 ; *C[C@H]1O[C@@H](n2cnc3c(N)nc(=O)[nH]c32)C[C@H]1*]

in an oligonucleotide chain unless specified otherwise). In some embodiments, it is b002C (which when utilized for a nucleoside refers to

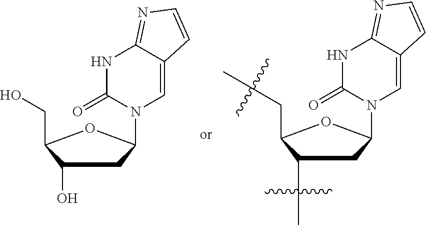 [SMILES: O=c1[nH]c2nccc-2cn1[C@H]1C[C@@H](O)[C@@H](CO)O1 ; *C[C@H]1O[C@@H](n2cc3ccnc-3[nH]c2=O)C[C@H]1*]

in an oligonucleotide chain unless specified otherwise). In some embodiments, it is b003C (which when utilized for a nucleoside refers to

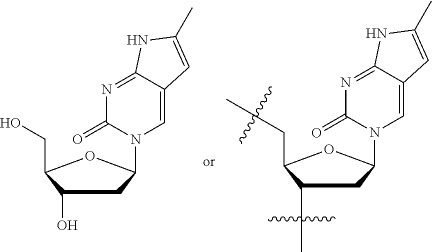 [SMILES: Cc1cc2cn([C@H]3C[C@@H](O)[C@@H](CO)O3)c(=O)nc2[nH]1 ; *C[C@H]1O[C@@H](n2cc3cc(C)[nH]c3nc2=O)C[C@H]1*]

in an oligonucleotide chain unless specified otherwise). In some embodiments, it is b003mC (which when utilized for a nucleoside refers to

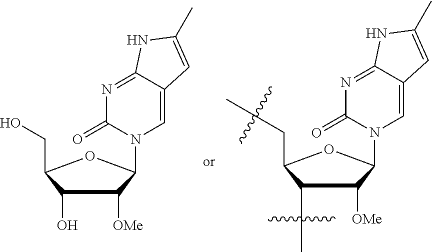

in an oligonucleotide chain unless specified otherwise). In some embodiments, it is b002I (which when utilized for a nucleoside refers to

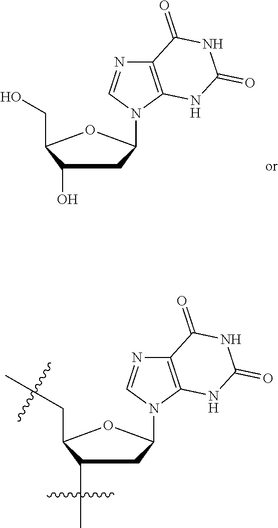 [SMILES: O=c1[nH]c(=O)c2ncn([C@H]3C[C@@H](O)[C@@H](CO)O3)c2[nH]1 ; *C[C@H]1O[C@@H](n2cnc3c(=O)[nH]c(=O)[nH]c32)C[C@H]1*]

in an oligonucleotide chain unless specified otherwise). In some embodiments, it is b003I (which when utilized for a nucleoside refers to

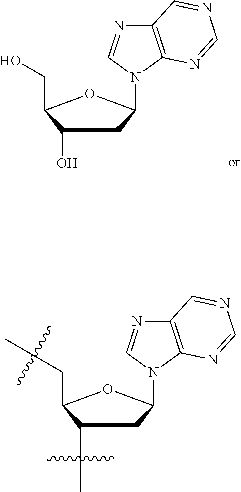 [SMILES: OC[C@H]1O[C@@H](n2cnc3cncnc32)C[C@H]1O ; *C[C@H]1O[C@@H](n2cnc3cncnc32)C[C@H]1*]

in an oligonucleotide chain unless specified otherwise). In some embodiments, it is Asm01 (which when utilized for a nucleoside refers to

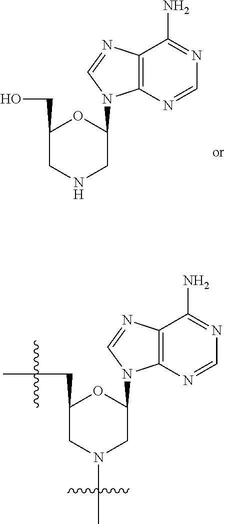 [SMILES: Nc1ncnc2c1ncn2[C@H]1CNC[C@@H](CO)O1 ; *C[C@@H]1CN(*)C[C@H](n2cnc3c(N)ncnc32)O1]

in an oligonucleotide chain unless specified otherwise; in some embodiments, the nitrogen atom is bonded to a linkage phosphorus). In some embodiments, it is Gsm01 (which when utilized for a nucleoside refers to

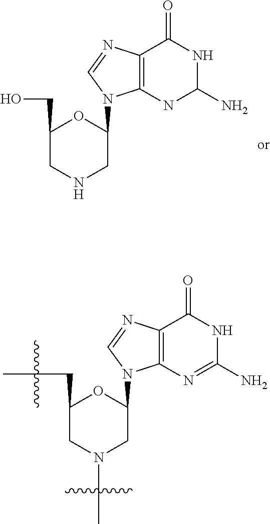 [SMILES: NC1NC(=O)c2ncn([C@H]3CNC[C@@H](CO)O3)c2N1 ; *C[C@@H]1CN(*)C[C@H](n2cnc3c(=O)[nH]c(N)nc32)O1]

in an oligonucleotide chain unless specified otherwise; in some embodiments, the nitrogen atom is bonded to a linkage phosphorus). In some embodiments, it is Tsm01 (which when utilized for a nucleoside refers to

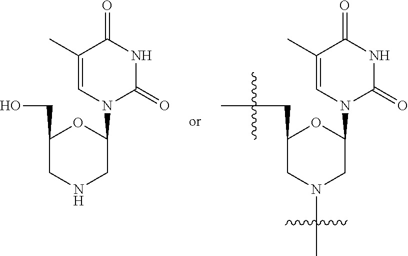 [SMILES: Cc1cn([C@H]2CNC[C@@H](CO)O2)c(=O)[nH]c1=O ; *C[C@@H]1CN(*)C[C@H](n2cc(C)c(=O)[nH]c2=O)O1]

in an oligonucleotide chain unless specified otherwise; in some embodiments, the nitrogen atom is bonded to a linkage phosphorus). In some embodiments, it is 5MsfC (which when utilized for a nucleoside refers to

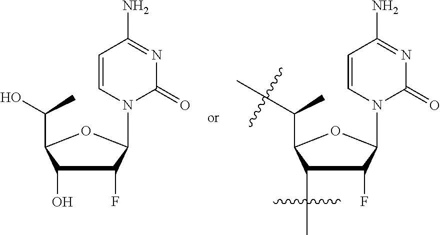

in an oligonucleotide chain unless specified otherwise). In some embodiments, it is Usm04 (which when utilized for a nucleoside refers to

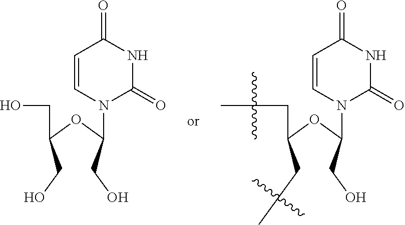

in an oligonucleotide chain unless specified otherwise). In some embodiments, it is 5MRdT (which when utilized for a nucleoside refers to

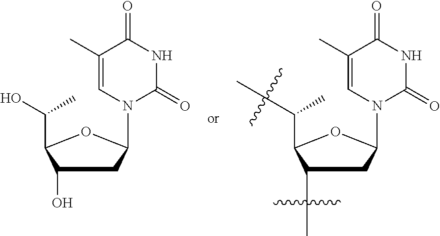

in an oligonucleotide chain unless specified otherwise). In some embodiments, it is Tsm18 (which when utilized for a nucleoside refers to

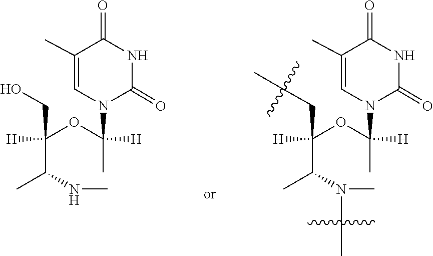 [SMILES: CN[C@H](C)[C@@H](CO)O[C@H](C)n1cc(C)c(=O)[nH]c1=O ; *C[C@@H](O[C@H](C)n1cc(C)c(=O)[nH]c1=O)[C@@H](C)N(*)C]

in an oligonucleotide chain unless specified otherwise; in some embodiments, the nitrogen atom is bonded to a linkage phosphorus). In some embodiments, it is b001Asm15 (which when utilized for a nucleoside refers to

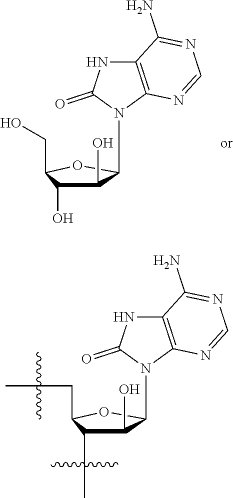

in an oligonucleotide chain unless specified otherwise). In some embodiments, it is Csm19 (which when utilized for a nucleoside refers to

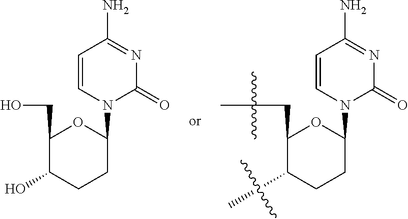

in an oligonucleotide chain unless specified otherwise). In some embodiments, it is b008Usm19 (which when utilized for a nucleoside refers to

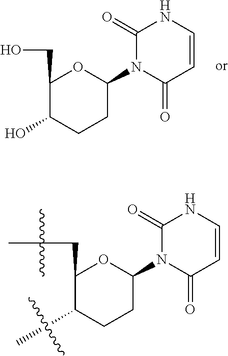

in an oligonucleotide chain unless specified otherwise).
In some embodiments, it is b006A (which when utilized for a nucleoside refers to

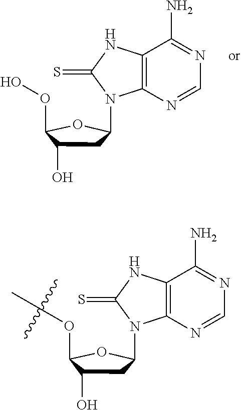

in an oligonucleotide chain unless specified otherwise). In some embodiments, it is b014U (which when utilized for a nucleoside refers to

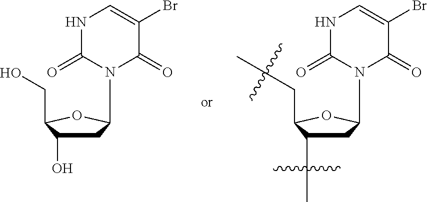

in an oligonucleotide chain unless specified otherwise). In some embodiments, it is b008Usm15 (which when utilized for a nucleoside refers to

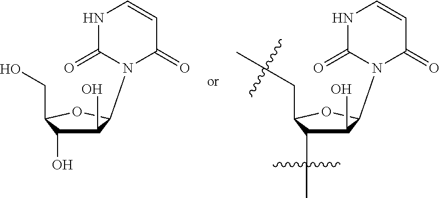

in an oligonucleotide chain unless specified otherwise). In some embodiments, it is b004A (which when utilized for a nucleoside refers to

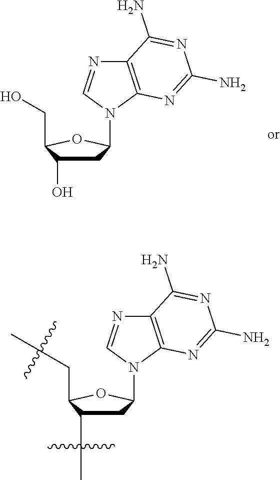

in an oligonucleotide chain unless specified otherwise). In some embodiments, it is b005A (which when utilized for a nucleoside refers to

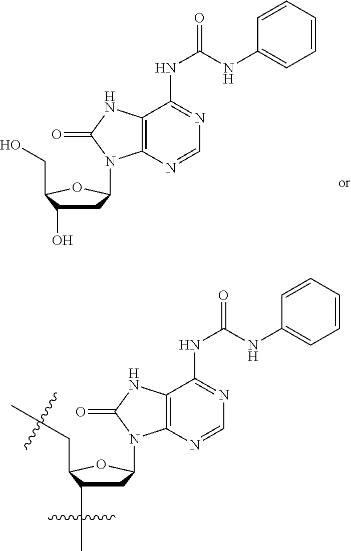

in an oligonucleotide chain unless specified otherwise).
In some embodiments, it is b015U (which when utilized for a nucleoside refers to

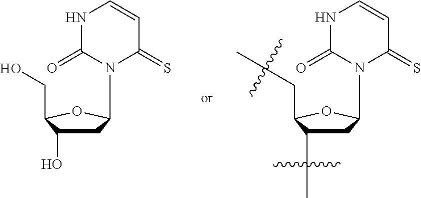

in an oligonucleotide chain unless specified otherwise). In some embodiments, N₀ is abasic. In some embodiments, N₀ is L010.

In some embodiments, certain modified nucleosides or nucleobases, e.g., b001A, b008U etc., can provide improved editing, e.g., when compared to dC at positions opposite to target adenosines. In some embodiments, certain nucleosides, e.g., dC, b001A, b001rA, Csm15, b001C, etc. can provide improved adenosine editing efficiency when utilized at N₀ compared to a reference nucleoside (e.g., under comparable conditions including in otherwise identical oligonucleotides, assessed in identical or comparable assays, etc.). In some embodiments, N₀ is b001A. In some embodiments, N₀ is b001Asm15. In some embodiments, N₀ is b001rA. In some embodiments, N₀ is b002A. In some embodiments, N₀ is b003A. In some embodiments, N₀ is b004A. In some embodiments, N₀ is b005A. In some embodiments, N₀ is b006A. In some embodiments, N₀ is b0041. In some embodiments, N₀ is b0141. In some embodiments, N₀ is b002G. In some embodiments, N₀ is dC. In some embodiments, N₀ is b001C. In some embodiments, N₀ is b009U. In some embodiments, No is b010U. In some embodiments, N₀ is b011U. In some embodiments, N₀ is b012U. In some embodiments, N₀ is b013U. In some embodiments, N₀ is b014U. In some embodiments, N₀ is b015U. In some embodiments, N₀ is b008Usm15. In some embodiments, N₀ is b008Usm19. In some embodiments, N₀ is Csm04. In some embodiments, N₀ is Csm11. In some embodiments, N₀ is Csm12. In some embodiments, N₀ is Csm15. In some embodiments, N₀ is Csm19. In some embodiments, N₀ is b009Csml1. In some embodiments, N₀ is b009Csm12. In some embodiments, N₀ is Gsm11. In some embodiments, N₀ is Gsm12. In some embodiments, N₀ is Tsm11. In some embodiments, N₀ is Tsm12. In some embodiments, a reference nucleoside is rU. In some embodiments, a reference nucleoside is dU. In some embodiments, a reference nucleoside is dT. In some embodiments, at N₀ position there is no nucleobase. In some embodiments, at N₀ position it is L010. In some embodiments, sugar of N₀ is sm15.

In some embodiments, replacing guanine with hypoxanthine at position −1 (e.g., replacing dG with dI) can provide improved editing.

In some embodiments, an oligonucleotide comprises 5′-N₁N₀N₋₁-3′ wherein each of N₁, N₀, and N₋₁ is independently a nucleoside as described herein. In some embodiments, an oligonucleotide comprises 5′-N₂N₁N₀N₋₁N₋₂-3′ wherein each of N₂, N₁, N₀, N₋₁, and N₋₂ is independently a nucleoside as described herein. In some embodiments, an oligonucleotide comprises 5′-N₃N₂N₁N₀N₋₁N₋₂N₋₃-3′ wherein each of N₃, N₂, N₁, N₀, N₋₁, N₋₂, and N₋₃ is independently a nucleoside as described herein. In some embodiments, an oligonucleotide comprises 5′-N₄N₃N₂N₁N₀N₋₁N₋₂N₋₃N₋₄-3′ wherein each of N₄, N₃, N₂, N₁, N₀, N₋₁, N₋₂, N₋₃, and N₋₄ is independently a nucleoside as described herein. In some embodiments, an oligonucleotide comprises 5′-N₅N₄N₃N₂N₁N₀N₋₁N₋₂N₋₃N₋₄N₋₅-3′ wherein each of N₅, N₄, N₃, N₂, N₁, N₀, N₋₁, N₋₂, N₋₃, N₋₄, and N₋₅ is independently a nucleoside as described herein. In some embodiments, an oligonucleotide comprises 5′-N₆N₅N₄N₃N₂N₁N₀N₋₁N₋₂N₋₃N₋₄N₋₅N₋₆-3′ wherein each of N₆, N₅, N₄, N₃, N₂, N₁, N₀, N₋₁, N₋₂, N₋₃, N₋₄, N₋₅, and N₋₆ is independently a nucleoside as described herein. In some embodiments, Nₙ wherein n is a positive number, e.g., N₁, may also be referred to as N₋ₙ, e.g., N₊₁ for N₁. In some embodiments, such an oligonucleotide can form a duplex with a nucleic acid (e.g., a RNA nucleic acid) and can edit a target adenosine which is opposite to N₀. In some embodiments, N₋₆ is the last nucleoside of an oligonucleotide (counting from the 5′-end).

In some embodiments, an oligonucleotide comprises 5′-N₂N₁N₀N₋₁N₋₂-3′, wherein each of N₂, N₁, N₀, N₋₁, and N₋₂ is independently a nucleoside. In some embodiments, an oligonucleotide comprises 5′-N₂N₁N₀N₋₁N₋₂-3′, wherein each of N₂, N₁, N₀, N₋₁, and N₋₂ is independently a nucleoside. In some embodiments, an oligonucleotide comprises 5′-N₂N₁N₀N₋₁N₋₂-3′, wherein each of N₂, N₁, N₀, N₋₁, and N₋₂ is independently a nucleoside, N₀ is opposite to a target adenosine, and each two of N₂, N₁, N₀, N₋₁, and N₋₂ that are next to each other, as those skilled in the art will appreciate, independently bond to an internucleotidic linkage as described herein. In some embodiments, one or more or all of N₁, N₀, and N₋₁ independently have a natural RNA sugar. In some embodiments, one or more or all of N₁, N₀, and N₋₁ independently have a natural DNA sugar. In some embodiments, the sugar of each of N₁, N₀, and N₋₁ is independently a natural DNA sugar or a 2′-F modified sugar. In some embodiments, the sugar of each of N₁, N₀, and N₋₁ is independently a natural DNA sugar. In some embodiments, the sugar of N₁ is a 2′-modified sugar, and the sugar of each of No and N₋₁ is independently a natural DNA sugar. In some embodiments, the sugar of N₁ is a 2′-F sugar, and the sugar of each of No and N₋₁ is independently a natural DNA sugar. In some embodiments, the sugar of N₁ is a modified sugar. In some embodiments, the sugar of N₁ is a 2′-F modified sugar. In some embodiments, the sugar of N₁ is a natural DNA sugar. In some embodiments, the sugar of N₁ is a natural RNA sugar. In some embodiments, the sugar of No is not a modified sugar. In some embodiments, the sugar of N₀ is not a 2′-modified sugar. In some embodiments, the sugar of N₀ is not a 2′-OR modified sugar, wherein R is optionally substituted C₁₋₆ alkyl. In some embodiments, the sugar of N₀ is not a 2′-F modified sugar. In some embodiments, the sugar of N₀ is not a 2′-OMe modified sugar. In some embodiments, the sugar of N₀ is a natural DNA or RNA sugar. In some embodiments, the sugar of N₀ is a natural DNA sugar. In some embodiments, the sugar of N₀ is a natural RNA sugar. In some embodiments, the sugar of N₋₁ is not a modified sugar. In some embodiments, the sugar of N₋₁ is not a 2′-modified sugar. In some embodiments, the sugar of N₋₁ is not a 2′-OR modified sugar, wherein R is optionally substituted C₁₋₆ alkyl. In some embodiments, the sugar of N₋₁ is not a 2′-F modified sugar. In some embodiments, the sugar of N₋₁ is not a 2′-OMe modified sugar. In some embodiments, the sugar of N₋₁ is a natural DNA or RNA sugar. In some embodiments, the sugar of N₋₁ is a natural DNA sugar. In some embodiments, the sugar of N₋₁ is a natural RNA sugar. In some embodiments, each of N₁, N₀ and N₋₁ independently has a natural RNA sugar. In some embodiments, each of N₁, No and N₋₁ independently has a natural DNA sugar. In some embodiments, N₁ has a 2′-F modified sugar, and each of N₀ and N₋₁ independently has a natural DNA or RNA sugar. In some embodiments, N₁ has a 2′-F modified sugar, and each of N₀ and N₋₁ independently has a natural DNA sugar. In some embodiments, two of N₁, N₀, and N₋₁ independently have a natural DNA or RNA sugar. In some embodiments, two of N₁, N₀, and N₋₁ independently have a natural DNA sugar. In some embodiments, each of N₁ and N₀ independently has a 2′-F modified sugar, and N₋₁ is a natural DNA sugar.

In some embodiments, such oligonucleotides provide high editing levels. In some embodiments, each of the two internucleotidic linkages bonded to N₋₁ is independently Rp. In some embodiments, each of the two internucleotidic linkages bonded to N₋₁ is independently an Rp phosphorothioate internucleotidic linkage. In some embodiments, each of the two internucleotidic linkages bonded to N₋₁ is independently an Rp phosphorothioate internucleotidic linkage, and each other phosphorothioate internucleotidic linkage in an oligonucleotide, if any, is independently Sp. In some embodiments, a 5′ internucleotidic linkage bonded to Ni is Rp. In some embodiments, an internucleotidic linkage bonded to N₁ and N₀(i.e., a 3′ internucleotidic linkage bonded to N₁) is Rp. In some embodiments, an internucleotidic linkage bonded to N₋₁ and N₀ is Rp. In some embodiments, a 3′ internucleotidic linkage bonded to N₋₁ is Rp. In some embodiments, each internucleotidic linkage bonded to N₀ is independently Rp. In some embodiments, each internucleotidic linkage bonded to N₀ or N₁ is independently Rp. In some embodiments, each internucleotidic linkage bonded to N₀ or N₋₁ is independently Rp. In some embodiments, each internucleotidic linkage bonded to N₁ is independently Rp. In some embodiments, each Rp internucleotidic linkage is independently an Rp phosphorothioate internucleotidic linkage. In some embodiments, each other chirally controlled phosphorothioate internucleotidic linkage in an oligonucleotide is independently Sp. In some embodiments, the internucleotidic linkage between N₀N₋₁ is Rp. In some embodiments, the internucleotidic linkage between N₀N₋₁ is Rp phosphorothioate internucleotidic linkage. In some embodiments, the internucleotidic linkage between N₋₁N₋₂ is Rp. In some embodiments, the internucleotidic linkage between N₋₁N₋₂ is Rp phosphorothioate internucleotidic linkage. In some embodiments, all internucleotidic linkages bonded to N₁, N₀, and N₋₁ are independently Sp. In some embodiments, all internucleotidic linkages bonded to N₁, N₀, and N₋₁ are independently Sp phosphorothioate internucleotidic linkages. In some embodiments, all internucleotidic linkages bonded to N₂, N₁, N₀, N₋₁, and N₋₂ are independently Sp. In some embodiments, all internucleotidic linkages bonded to N₂, N₁, N₀, N₋₁, and N₋₂ are independently Sp phosphorothioate internucleotidic linkages. In some embodiments, both internucleotidic linkage bonded to N₁ are independently Sp (e.g., Sp phosphorothioate internucleotidic linkages). In some embodiments, an internucleotidic linkage between N₁ and N₀ is Sp (e.g., a Sp phosphorothioate internucleotidic linkage). In some embodiments, an internucleotidic linkage between N₋₁ and No is Sp (e.g., a Sp phosphorothioate internucleotidic linkage). In some embodiments, an internucleotidic linkage between N₋₁ and N₋₂ is a neutral internucleotidic linkage. In some embodiments, an internucleotidic linkage between N₋₁ and N₋₂ is a PN linkage. In some embodiments, an internucleotidic linkage between N₋₁ and N₋₂ is a non-negatively charged internucleotidic linkage. In some embodiments, an internucleotidic linkage between N₋₁ and N₋₂ is a phosphoryl guanidine linkage. In some embodiments, an internucleotidic linkage between N₋₁ and N₋₂ is n001. In some embodiments, an internucleotidic linkage between N₋₁ and N₋₂ is not chirally controlled. In some embodiments, an internucleotidic linkage between N₋₁ and N₋₂ is chirally controlled. In some embodiments, an internucleotidic linkage between N₋₁ and N₋₂ is Rp. In some embodiments, an internucleotidic linkage between N₋₁ and N₋₂ is Sp. In some embodiments, N₂ comprises a modified sugar. In some embodiments, N₋₂ comprises a modified sugar. In some embodiments, each of N₂ and N₋₂ independently comprises a modified sugar. In some embodiments, a modified sugar is a 2′-modified sugar. In some embodiments, a modified sugar is 2′-OR modified sugar, wherein R is optionally substituted C₁₋₆ aliphatic. In some embodiments, a 2′-modified sugar is 2′-OMe modified sugar. In some embodiments, a 2′-modified sugar is 2′-MOE modified sugar. In some embodiments, a modified sugar is a bicyclic sugar, e.g., a LNA sugar, a cEt sugar, etc.

In some embodiments, to the 3′-side of a nucleoside opposite to a target adenosine (e.g., No) there are at least 2, 3, 4, 5, 6, 7, 8, 9 or more nucleosides (e.g., 2-30, 3-30, 4-30, 5-30, 2-20, 3-20, 4-20, 5-20, 2-15, 3-15, 4-15, 5-15, 2-10, 3-10, 4-10, 5-10, 2, 3, 4, 5, 6, 7, 8, 9, 10, 11, 12, 13, 14, 15, 16, 17, 18, 19, or 20, etc., “3′-side nucleosides”). In some embodiments, there are at least 2 3′-side nucleosides. In some embodiments, there are at least 3 3′-side nucleosides. In some embodiments, there are at least 4 3′-side nucleosides. In some embodiments, there are at least 5 3′-side nucleosides (e.g., an oligonucleotide comprising 5′-N₀N₋₁N₋₂N₋₃N₋₄N₋₅-3′, wherein each of N₀, N₋₁, N₋₂, N₋₃, N₋₄, and N₋₅ is independently a nucleoside). In some embodiments, there are at least 6 3′-side nucleosides (e.g., an oligonucleotide comprising 5′-N₀N₋₁N₋₂N₋₃N₋₄N₋₅N₋₆-3′, wherein each of N₀, N₋₁, N₋₂, N₋₃, N₋₄, N₋₅, and N₋₆ is independently a nucleoside). In some embodiments, there are at least 7 3′-side nucleosides. In some embodiments, there are at least 8 3′-side nucleosides. In some embodiments, there are at least 9 3′-side nucleosides. In some embodiments, there are at least 10 3′-side nucleosides. In some embodiments, there are 2 3′-side nucleosides. In some embodiments, there are 3 3′-side nucleosides. In some embodiments, there are 4 3′-side nucleosides. In some embodiments, there are 5 3′-side nucleosides. In some embodiments, there are 6 3′-side nucleosides. In some embodiments, there are 7 3′-side nucleosides. In some embodiments, there are 8 3′-side nucleosides. In some embodiments, there are 9 3′-side nucleosides. In some embodiments, there are 10 3′-side nucleosides. In some embodiments, to the 5′-side of a nucleoside opposite to a target adenosine (e.g., N₀) there are at least 15, 16, 17, 18, 19, 20, 21, 22, 23, 24, 25, 26, 27, 28, 29 or 30 or more nucleosides (e.g., 15-50, 20-50, 21-50, 22-50, 23-50, 24-50, 25-50, 26-50, 27-50, 28-50, 29-50, 30-50, 15-40, 20-40, 21-40, 22-40, 23-40, 24-40, 25-40, 26-40, 27-40, 28-40, 29-40, 30-40, 15-30, 20-30, 21-30, 22-30, 23-30, 24-30, 25-30, 26-30, 27-30, 28-30, 29-30, 15, 16, 17, 18, 19, 20, 21, 22, 23, 24, 25, 26, 27, 28, 29 or 30, etc.). In some embodiments, there are at least 15 5′-side nucleosides. In some embodiments, there are at least 16 5′-side nucleosides. In some embodiments, there are at least 17 5′-side nucleosides. In some embodiments, there are at least 18 5′-side nucleosides. In some embodiments, there are at least 19 5′-side nucleosides. In some embodiments, there are at least 20 5′-side nucleosides. In some embodiments, there are at least 21 5′-side nucleosides. In some embodiments, there are at least 22 5′-side nucleosides. In some embodiments, there are at least 23 5′-side nucleosides. In some embodiments, there are at least 24 5′-side nucleosides. In some embodiments, there are at least 25 5′-side nucleosides. In some embodiments, there are at least 26 5′-side nucleosides. In some embodiments, there are at least 27 5′-side nucleosides. In some embodiments, there are at least 28 5′-side nucleosides. In some embodiments, there are at least 29 5′-side nucleosides. In some embodiments, there are at least 30 5′-side nucleosides. In some embodiments, there are 15 5′-side nucleosides. In some embodiments, there are 16 5′-side nucleosides. In some embodiments, there are 17 5′-side nucleosides. In some embodiments, there are 18 5′-side nucleosides. In some embodiments, there are 19 5′-side nucleosides. In some embodiments, there are 20 5′-side nucleosides. In some embodiments, there are 21 5′-side nucleosides. In some embodiments, there are 22 5′-side nucleosides. In some embodiments, there are 23 5′-side nucleosides. In some embodiments, there are 24 5′-side nucleosides. In some embodiments, there are 25 5′-side nucleosides. In some embodiments, there are 26 5′-side nucleosides. In some embodiments, there are 27 5′-side nucleosides. In some embodiments, there are 28 5′-side nucleosides. In some embodiments, there are 29 5′-side nucleosides. In some embodiments, there are 30 5′-side nucleosides. In some embodiments, there are at least 4 3′-side nucleosides and at least 22 5′-side nucleosides. In some embodiments, there are at least 4 3′-side nucleosides and at least 23 5′-side nucleosides. In some embodiments, there are at least 4 3′-side nucleosides and at least 24 5′-side nucleosides. In some embodiments, there are at least 4 3′-side nucleosides and at least 25 5′-side nucleosides. In some embodiments, there are at least 5 3′-side nucleosides and at least 22 5′-side nucleosides. In some embodiments, there are at least 5 3′-side nucleosides and at least 23 5′-side nucleosides. In some embodiments, there are at least 5 3′-side nucleosides and at least 24 5′-side nucleosides. In some embodiments, there are at least 5 3′-side nucleosides and at least 25 5′-side nucleosides. In some embodiments, there are at least 6 3′-side nucleosides and at least 21 5′-side nucleosides. In some embodiments, there are at least 6 3′-side nucleosides and at least 22 5′-side nucleosides. In some embodiments, there are at least 6 3′-side nucleosides and at least 23 5′-side nucleosides. In some embodiments, there are at least 6 3′-side nucleosides and at least 24 5′-side nucleosides. In some embodiments, there are at least 7 3′-side nucleosides and at least 20 5′-side nucleosides. In some embodiments, there are at least 7 3′-side nucleosides and at least 21 5′-side nucleosides. In some embodiments, there are at least 7 3′-side nucleosides and at least 22 5′-side nucleosides. In some embodiments, there are at least 7 3′-side nucleosides and at least 23 5′-side nucleosides. In some embodiments, there are at least 8 3′-side nucleosides and at least 19 5′-side nucleosides. In some embodiments, there are at least 8 3′-side nucleosides and at least 20 5′-side nucleosides. In some embodiments, there are at least 8 3′-side nucleosides and at least 21 5′-side nucleosides. In some embodiments, there are at least 8 3′-side nucleosides and at least 22 5′-side nucleosides. In some embodiments, there are at least 9 3′-side nucleosides and at least 18 5′-side nucleosides. In some embodiments, there are at least 9 3′-side nucleosides and at least 19 5′-side nucleosides. In some embodiments, there are at least 9 3′-side nucleosides and at least 20 5′-side nucleosides. In some embodiments, there are at least 9 3′-side nucleosides and at least 21 5′-side nucleosides. In some embodiments, there are at least 10 3′-side nucleosides and at least 17 5′-side nucleosides. In some embodiments, there are at least 10 3′-side nucleosides and at least 18 5′-side nucleosides. In some embodiments, there are at least 10 3′-side nucleosides and at least 19 5′-side nucleosides. In some embodiments, there are at least 10 3′-side nucleosides and at least 20 5′-side nucleosides. In some embodiments, there are at least 11 3′-side nucleosides and at least 16 5′-side nucleosides. In some embodiments, there are at least 11 3′-side nucleosides and at least 17 5′-side nucleosides. In some embodiments, there are at least 11 3′-side nucleosides and at least 18 5′-side nucleosides. In some embodiments, there are at least 11 3′-side nucleosides and at least 19 5′-side nucleosides. In some embodiments, there are at least 12 3′-side nucleosides and at least 15 5′-side nucleosides. In some embodiments, there are at least 12 3′-side nucleosides and at least 16 5′-side nucleosides. In some embodiments, there are at least 12 3′-side nucleosides and at least 17 5′-side nucleosides. In some embodiments, there are at least 12 3′-side nucleosides and at least 18 5′-side nucleosides. In some embodiments, there are at least 13 3′-side nucleosides and at least 14 5′-side nucleosides. In some embodiments, there are at least 13 3′-side nucleosides and at least 15 5′-side nucleosides. In some embodiments, there are at least 13 3′-side nucleosides and at least 16 5′-side nucleosides. In some embodiments, there are at least 13 3′-side nucleosides and at least 17 5′-side nucleosides. In some embodiments, certain useful lengths of 5′-sides and/or 3′-sides and/or positioning of nucleosides opposite to target adenosines are described in WO 2021/071858 and/or WO 2022/099159 and are incorporated herein by reference.

As described herein, wherein modifications may be utilized for N₁, including sugar modifications, nucleobase modifications, etc. In some embodiments, N₁ contains a natural DNA sugar. In some embodiments, N₁ contains a natural RNA sugar. In some embodiments, N₁ contains a modified sugar as described herein. In some embodiments, a modified sugar is a 2′-modified sugar. In some embodiments, a modified sugar is a 2′-F modified sugar. In some embodiments, a modified sugar is a 2′-OR modified sugar wherein R is optionally substituted C₁₋₆ alkyl. In some embodiments, a modified sugar is a 2′-OMe modified sugar. In some embodiments, a modified sugar is a 2′-MOE modified sugar. In some embodiments, a sugar is a UNA sugar. In some embodiments, a sugar is a GNA sugar. In some embodiments, sugar of N₁ is sm01. In some embodiments, it is sm11. In some embodiments, it is sm12. In some embodiments, it is sm18. In some embodiments, a modified sugar, e.g., a 2′-F modified sugar, or a DNA sugar provides higher editing efficiency when administered to a system (e.g., a cell, a tissue, an organism, etc.) compared to a reference sugar (e.g., a natural RNA sugar, a different modified sugar, etc.). In some embodiments, N₁ contains a natural nucleobase, e.g., U. In some embodiments, N₁ contains a modified nucleobase as described herein. In some embodiments, nucleobase of N₁ is A, T, C, G, U, hypoxanthine, b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b0021, b0031, b0041, b014I, [3nT], [3ne5U], [3nfl5U], [3npry5U], [3ncn5U], [nathp6o8A], [ipr6o8A], [c7In], [c39z481n], [z2c3In], [z5C], or zdnp. In some embodiments, nucleobase of N₁ is T. In some embodiments, it is U. In some embodiments, it is b002A. In some embodiments, it is b003A. In some embodiments, it is b008U. In some embodiments, it is b010U. In some embodiments, it is b011U. In some embodiments, it is b012U. In some embodiments, it is b001C. In some embodiments, it is b004C. In some embodiments, it is b007C. In some embodiments, it is b008C. In some embodiments, N₁ is a natural nucleoside. In some embodiments, N₁ is a modified nucleoside. In some embodiments, N₁ is fU, dU, fA, dA, fT, dT, fC, dC, fG, dG, dI, fI, aC, b001U, b002U, b003U, b004U, b005U, b006U, b007U, b008U, b009U, b010U, b011U, b012U, b013U, b014U, b015U, b001A, b001rA, b002A, b003A, b004A, b005A, b006A, b007A, b001G, b002G, b001C, b002C, b003C, b003mC, b004C, b005C, b006C, b007C, b008C, b002I, b003I, b004I, b014I, Asm01, Gsm01, 5MSfC, Usm04, 5MRdT, Csm04, Csm11, Gsm11, Tsm11, b009Csm11, b009Csm12, Gsm12, Tsm12, Csm12, rCsm13, rCsm14, Csm15, Csm16, Csm17, L034, zdnp, and Tsm18. In some embodiments, N₁ is fU, dU, fA, dA, fT, dT, fC, dC, fG, dG, dI, or fI. In some embodiments, N₁ is fU, dU, fA, dA, fT, dT, fC, dC, fG, or dG. In some embodiments, N₁ is dT. In some embodiments, N₁ is b001A. In some embodiments, N₁ is b002A. In some embodiments, N₁ is b003A. In some embodiments, N₁ is b004A. In some embodiments, N₁ is b005A. In some embodiments, N₁ is b006A. In some embodiments, N₁ is fU. In some embodiments, N₁ is b008U. In some embodiments, N₁ is b001C. In some embodiments, N₁ is b004C. In some embodiments, N₁ is b007C. In some embodiments, N₁ is b008C. In some embodiments, N₁ is b001U. In some embodiments, N₁ is b008U. In some embodiments, N₁ is b010U. In some embodiments, N₁ is b011U. In some embodiments, N₁ is b012U. In some embodiments, N₁ is b013U. In some embodiments, N₁ is b014U. In some embodiments, N₁ is b015U. In some embodiments, N₁ is Csm11. In some embodiments. N₁ is Gsm11. In some embodiments, N₁ is Tsm11. In some embodiments, N₁ is b009Csm11. In some embodiments, N₁ is Csm12. In some embodiments, N₁ is Gsm12. In some embodiments, N₁ is Tsm12. In some embodiments, N₁ is b009Csm12. In some embodiments, N₁ is Gsm01. In some embodiments, N₁ is Tsm01. In some embodiments, N₁ is Csm17. In some embodiments, N₁ is Tsm18. In some embodiments, N₁ is b014I. In some embodiments, N₁ is abasic. In some embodiments, N₁ is L010. As described herein, at position N₁ in some embodiments, it is a match when an oligonucleotide forms a duplex with a nucleic acid (e.g., its target transcript for adenosine editing). In some embodiments, it is a mismatch. In some embodiments, it is a wobble. In some embodiments, N₁ is bonded to a natural phosphate linkage. In some embodiments, N₁ is bonded to a modified internucleotidic linkage as described herein, in various embodiments, with defined stereochemistry. In some embodiments, N₁ is bonded to a natural phosphate linkage and a modified internucleotidic linkage. In some embodiments, N₁ is bonded to two natural phosphate linkages. In some embodiments, N₁ is bonded to two modified internucleotidic linkages, each of which may be independently and optionally stereocontrolled and may be Rp or Sp.

As described herein, wherein modifications may be utilized for N₋₁, including sugar modifications, nucleobase modifications, etc. In some embodiments, N₋₁ contains a natural DNA sugar. In some embodiments, N₋₁ contains a natural RNA sugar. In some embodiments, N₋₁ contains a modified sugar as described herein. In some embodiments, a modified sugar is a 2′-modified sugar. In some embodiments, a modified sugar is a 2′-F modified sugar. In some embodiments, a modified sugar is a 2′-OR modified sugar wherein R is optionally substituted C₁₋₆ alkyl. In some embodiments, a modified sugar is a 2′-OMe modified sugar. In some embodiments, a modified sugar is a 2′-MOE modified sugar. In some embodiments, a sugar is a UNA sugar. In some embodiments, a sugar is a GNA sugar. In some embodiments, sugar of N₋₁ is sm01. In some embodiments, it is sm11. In some embodiments, it is sm12. In some embodiments, it is sm18. In some embodiments, a modified sugar, e.g., a 2′-F modified sugar, or a DNA sugar provides higher editing efficiency when administered to a system (e.g., a cell, a tissue, an organism, etc.) compared to a reference sugar (e.g., a natural RNA sugar, a different modified sugar, etc.). In some embodiments, N₋₁ contains a natural nucleobase, e.g., U. In some embodiments, N₋₁ contains a modified nucleobase as described herein. In some embodiments, nucleobase of N₋₁ is A, T, C, G, U, hypoxanthine, b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b003I, b004I, b014I, [3nT], [3ne5U], [3nfl5U], [3npry5U], [3ncn5U], [nathp6o8A], [ipr6o8A], [c7In], [c39z481n], [z2c3In], [z5C], or zdnp. In some embodiments, nucleobase of N₋₁ is T. In some embodiments, it is U. In some embodiments, it is b001A. In some embodiments, it is b002A. In some embodiments, it is b003A. In some embodiments, it is b004A. In some embodiments, it is b005A. In some embodiments, it is b006A. In some embodiments, it is b008U. In some embodiments, it is b011U. In some embodiments, it is b012U. In some embodiments, it is b013U. In some embodiments, it is b014U. In some embodiments. it is b015U. In some embodiments, it is b001C. In some embodiments, it is b004C. In some embodiments, it is b007C. In some embodiments, it is b008C. In some embodiments, it is b009C. In some embodiments, it is b002G. In some embodiments, it is b0141. In some embodiments, it is [3nT]. In some embodiments, it is [3ne5U]. In some embodiments, it is [3nfl5U]. In some embodiments, it is [3npry5U]. In some embodiments, it is [3ncn5U]. In some embodiments, it is [nathp608A]. In some embodiments, it is [ipr6o8A]. In some embodiments, it is [c7In]. In some embodiments, it is [c39z481n]. In some embodiments, it is [z2c3In]. In some embodiments, it is [z5C]. In some embodiments, with the high flexibility and efficiency of technologies described herein, various nucleobases and sugars can be utilized at N₋₁ to provide editing. For example, various nucleobases, e.g., G, b006C, b007C, b004A, b007A, b002I, b003I, b004I, etc., and/or various sugars, e.g., natural DNA sugars, sm11, sm12, etc., can be utilized at N₋₁, which is opposite to C, to provide editing, in some embodiments, with higher editing levels compared to certain other nucleobases and/or sugars; and various nucleobases, e.g., C, b008U, b005U, b009U, b012U, b005C, b008C, b001A, b004A, b007A, b002I, b004I, b001G, abasic, etc., and/or various sugars, e.g., natural DNA sugars, sm11, sm12, sm15, etc., can be utilized at N₋₁ which is opposite to G, to provide editing, in some embodiments, with higher editing levels compared to certain other nucleobases and/or sugars; and various nucleobases, e.g., A, b004U, b009U, b010U, b011U, b012U, b006C, b008C, b009C, b001A, b003A, b004A, b006A, b007A, b003I, b004I, b001G, abasic, etc., and/or various sugars, e.g., natural DNA sugars, sm11, sm12, sm15, etc., can be utilized at N₋₁, which is opposite to U/T, to provide editing, in some embodiments, with higher editing levels compared to certain other nucleobases and/or sugars; and various nucleobases, e.g., U, T, b003U, b004U, b005U, b008U, b009U, b010U, b011U, b012U, b001C, b002C, b003C, b004C, b005C, b006C, b007C, b008C, b001A, b003A, b004A, b006A, b007A, b002I, b003I, b004I, b001G, b002G, abasic, etc., and various sugars, e.g., natural DNA sugar, sm11, sm12, sm15, sm17, etc., can be utilized at N₋₁ which is opposite to A, to provide editing, in some embodiments, with higher editing levels compared to certain other nucleobases and/or sugars. In some embodiments, nucleobase G can be utilized to opposite C at N₋₁ to provide effective editing, e.g., when the nucleobase of N₀ is b008U. In some embodiments, N₀ is b008U and N₋₁ is G. In some embodiments, when a neighboring nucleoside (e.g., 5′-side) of a target adenosine is A, certain nucleobases, sugars, etc. at N₋₁ position (opposite to a 5′ neighboring adenosine) provide higher editing efficiency and/or selectivity compared to others. In some embodiments, certain nucleobases, e.g., b008U, at N₀ provide target adenosine editing with higher selectivity over a neighboring A (e.g., a 5′-side A) compared to other nucleobases, e.g., C. In some embodiments, N₀ is b008U to provide editing efficiency and selectivity. In some embodiments, N₋₁ is a natural nucleoside. In some embodiments, N₋₁ is a modified nucleoside. In some embodiments, N₋₁ is fU, dU, fA, dA, fT, dT, fC, dC, fG, dG, dI, fI, aC, b001U, b002U, b003U, b004U, b005U, b006U, b007U, b008U, b009U, b010U, b011U, b012U, b013U, b014U, b015U, b001A, b001rA, b002A, b003A, b004A, b005A, b006A, b007A, b001G, b002G, b001C, b002C, b003C, b003mC, b004C, b005C, b006C, b007C, b008C, b002I, b0031, b004I, b014I, Asm01, Gsm01, 5MSfC, Usm04, 5MRdT, Csm04, Csm11, Gsm11, Tsm11, b009Csm11, b009Csm12, Gsm12, Tsm12, Csm12, rCsm13, rCsm14, Csm15, Csm16, Csm17, L034, zdnp, and Tsm18. In some embodiments, N₋₁ is fU, dU, fA, dA, fT, dT, fC, dC, fG, dG, dI, or fI. In some embodiments, N₋₁ is fU, dU, fA, dA, fT, dT, fC, dC, fG, or dG. In some embodiments, N₋₁ is dI. In some embodiments, N₋₁ is rI. In some embodiments, N₋₁ is dT. In some embodiments, N₋₁ is b001A. In some embodiments, N₋₁ is b002A. In some embodiments, N₋₁ is b003A. In some embodiments, N₋₁ is b004A. In some embodiments, N₋₁ is b005A. In some embodiments, N₋₁ is b006A. In some embodiments, N₋₁ is b007A. In some embodiments, N₋₁ is fU. In some embodiments, N₋₁ is b001C. In some embodiments, N₋₁ is b002C. In some embodiments, N₋₁ is b003C. In some embodiments, N₋₁ is b004C. In some embodiments, N₋₁ is b005C. In some embodiments, N₋₁ is b006C. In some embodiments, N₋₁ is b007C. In some embodiments, N₋₁ is b008C. In some embodiments, N₋₁ is b009Csm12. In some embodiments, N₋₁ is b001G. In some embodiments, N₋₁ is b002G. In some embodiments, N₋₁ is b001U. In some embodiments, N₋₁ is b003U. In some embodiments, N₋₁ is b004U. In some embodiments, N₋₁ is b005U. In some embodiments, N₋₁ is b006U. In some embodiments, N₋₁ is b007U. In some embodiments, N₋₁ is b008U. In some embodiments, N₋₁ is b009U. In some embodiments, N₋₁ is b010U. In some embodiments, N₋₁ is b011U. In some embodiments, N₋₁ is b012U. In some embodiments, N₋₁ is b013U. In some embodiments, N₋₁ is b014U. In some embodiments, N₋₁ is b015U. In some embodiments, N₋₁ is b002I. In some embodiments, N₋₁ is b003I. In some embodiments, N₋₁ is b004I. In some embodiments, N₋₁ is Gsm01. In some embodiments, N₋₁ is Tsm01. In some embodiments, N₋₁ is Csm11. In some embodiments, N₋₁ is b009Csm11. In some embodiments, N₋₁ is Gsm11. In some embodiments, N₋₁ is Tsm11. In some embodiments, N₋₁ is Csm12. In some embodiments, N₋₁ is b009Csm12. In some embodiments, N₋₁ is Gsm12. In some embodiments, N₋₁ is Tsm12. In some embodiments, N₋₁ is Csm17. In some embodiments, N₋₁ is Tsm18. In some embodiments, N₋₁ is abasic. In some embodiments, N₋₁ is L010. In some embodiments, N₋₁ is b002G. In some embodiments, N₋₁ is b014I. As described herein, at position N₋₁ in some embodiments, it is a match when an oligonucleotide forms a duplex with a nucleic acid (e.g., its target transcript for adenosine editing). In some embodiments, it is a mismatch. In some embodiments, it is a wobble. In some embodiments, N₋₁ is bonded to a natural phosphate linkage. In some embodiments, N₋₁ is bonded to a modified internucleotidic linkage as described herein, in various embodiments, with defined stereochemistry. In some embodiments, N₋₁ is bonded to a natural phosphate linkage and a modified internucleotidic linkage. In some embodiments, N₋₁ is bonded to two natural phosphate linkages. In some embodiments, N₋₁ is bonded to two modified internucleotidic linkages, each of which may be independently and optionally stereocontrolled and may be Rp or Sp.

In some embodiments, N₂ contains a natural sugar. In some embodiments, sugar of N₂ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₁ and N₂ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₃ contains a natural sugar. In some embodiments, sugar of N₃ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₂ and N₃ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001. In some embodiments, N₄ contains a natural sugar. In some embodiments, sugar of N₄ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₃ and N₄ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₅ contains a natural sugar. In some embodiments, sugar of N₅ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₄ and N₁ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₆ contains a natural sugar. In some embodiments, sugar of N₆ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₅ and N₆ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

As described herein, an oligonucleotide, or a portion thereof, e.g., a first domain, a second domain, etc., may comprise or consist of one or more, e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc. blocks, each of which independently comprises one or more (e.g., 1-50, 1-40, 1-30, 1-25, 1-24, 1-23, 1-22, 1-21, 1-20, 1-10, 1-5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) sugars, wherein each sugar in a block share the same structure. In some embodiments, an oligonucleotide, or a portion thereof, e.g., a first domain, a second domain, etc., may comprise or consist of one or more, e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc. blocks, each of which independently comprises one or more (e.g., 1-50, 1-40, 1-30, 1-25, 1-24, 1-23, 1-22, 1-21, 1-20, 1-10, 1-5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) sugars, wherein each sugar in a block is the same modified sugar. In some embodiments, each block independently contains 1-10, e.g., 1, 2, 3, 4, 5, 6, 7, 8, 9, or 10, sugars. In some embodiments, each block independently contains 1-5 sugars. In some embodiments, each block independently contains 1, 2, or 3 sugars. In some embodiments, one or more blocks, e.g., 1-15, 1-10, 1, 2, 3, 4, 5, 6, 7, 8, 9, 10, 11, 12, 13, 14, 15, 16, 17, 18, 19, 20 or more, independently contain two or three or more sugars. In some embodiments, one or more blocks, e.g., 1-15, 1-10, 1, 2, 3, 4, 5, 6, 7, 8, 9, 10, 11, 12, 13, 14, 15, 16, 17, 18, 19, 20 or more, independently contain two or three sugars. In some embodiments, about or at least about 30%, 40% or 50% blocks in an oligonucleotide or a portion thereof independently contains two or more (e.g., two or three) sugars. In some embodiments, about 50% blocks in an oligonucleotide of a first domain independently contains two or more (e.g., two or three) sugars. In some embodiments, a block is a 2′-F block wherein each sugar in the block is a 2′-F modified block. In some embodiments, a block is a 2′-OR block wherein R is optionally substituted C₁₋₆ aliphatic wherein each sugar in the block is the same 2′-OR modified sugar. In some embodiments, a block is a 2′-OMe block. In some embodiments, a block is a 2′-MOE block. In some embodiments, a block is a bicyclic sugar block wherein each sugar in the block is the same bicyclic sugar (e.g., a LNA sugar, cEt, etc.). In some embodiments, two or more blocks are 2′-F blocks. In some embodiments, every other block is a 2′-F block. In some embodiments, each 2′-F block independently contains no more than 2, 3, 4, 5, 6, 7, 8, 9, or 10 sugars. In some embodiments, a 2′-F block contains no more than 5 sugars. In some embodiments, a 2′-F block contains no more than 4 sugars. In some embodiments, a 2′-F block contains no more than 3 sugars. In some embodiments, between every two 2′-F blocks in an oligonucleotide or a portion thereof there is at least one 2′-OR block wherein R is optionally substituted C₁₋₆ aliphatic or one bicyclic sugar block. In some embodiments, between every two 2′-F blocks in a portion there is at least one 2′-OR block wherein R is optionally substituted C₁₋₆ aliphatic or one bicyclic sugar block. In some embodiments, between every two 2′-F blocks in an oligonucleotide there is at least one 2′-OR block wherein R is optionally substituted C₁₋₆ aliphatic. In some embodiments, between every two 2′-F blocks in a first domain there is at least one 2′-OR block wherein R is optionally substituted C₁₋₆ aliphatic. In some embodiments, between every two 2′-F blocks in a first domain there is at least one 2′-OMe block. In some embodiments, between two 2′-F blocks in a first domain there is a 2′-OMe block. In some embodiments, between two 2′-F blocks in a first domain there is a 2′-MOE block. In some embodiments, between two 2′-F blocks in a first domain there is a 2′-MOE block and 2′-OMe block. In some embodiments, between two 2′-F blocks in a first domain there is a 2′-MOE block and 2′-OMe block and no 2′-F block. In some embodiments, each 2′-F block is independently bonded to a 2′-OR block wherein R is C₁₋₆ aliphatic or a bicyclic sugar block. In some embodiments, each 2′-F block is independently bonded to a 2′-OR block wherein R is C₁₋₆ aliphatic. In some embodiments, each block a 2′-F block bonds to is independently a 2′-OR block wherein R is optionally substituted C₁₋₆ aliphatic or a bicyclic sugar block. In some embodiments, each block a 2′-F block bonds to is independently a 2′-OR block wherein R is optionally substituted C₁₋₆ aliphatic. In some embodiments, each block in a first domain that a 2′-F block in a first domain bonds to is independently a 2′-OR block wherein R is optionally substituted C₁₋₆ aliphatic or a bicyclic sugar block. In some embodiments, each block in a first domain that a 2′-F block in a first domain bonds to is independently a 2′-OR block wherein R is optionally substituted C₁₋₆ aliphatic. In some embodiments, each block in a first domain that a 2′-OR block wherein R is C₁₋₆ aliphatic or a bicyclic sugar block bonds is independently a 2′-F block of a different 2′-OR block wherein R is C₁₋₆ aliphatic or a bicyclic sugar block. In some embodiments, each block in a first domain that a 2′-OR block wherein R is C₁₋₆ aliphatic bonds is independently a 2′-F block of a different 2′-OR block wherein R is C₁₋₆ aliphatic. In some embodiments, a 2′-OR block is a 2′-OMe block. In some embodiments, a 2′-OR block is a 2′-MOE block. In some embodiments, at least one block is a 2′-OMe block. In some embodiments, about or about at least 2, 3, 4, or 5 blocks are independently 2′-OMe block. In some embodiments, at least one block is a 2′-MOE block. In some embodiments, about or about at least 2, 3, 4, or 5 blocks are independently 2′-MOE block. In some embodiments, in an oligonucleotide or a portion thereof, e.g., a first domain, a second domain, etc., there are one or more (e.g., 1, 2, 3, 4, 5, 6, 7, 8, 9, or 10 or more) 2′-OMe block and one or more (e.g., 1, 2, 3, 4, 5, 6, 7, 8, 9, or 10 or more) 2′-MOE block. In some embodiments, in an oligonucleotide or a portion thereof, e.g., a first domain, a second domain, etc., there are one or more (e.g., 1, 2, 3, 4, 5, 6, 7, 8, 9, or 10 or more) 2′-OMe block and one or more (e.g., 1, 2, 3, 4, 5, 6, 7, 8, 9, or 10 or more) 2′-MOE block and one or more (e.g., 1, 2, 3, 4, 5, 6, 7, 8, 9, or 10) 2′-F block. In some embodiments, in a first domain there are one or more (e.g., 1, 2, 3, 4, 5, 6, 7, 8, 9, or 10 or more) 2′-OMe block and one or more (e.g., 1, 2, 3, 4, 5, 6, 7, 8, 9, or 10 or more) 2′-MOE block. In some embodiments, in a first domain there are one or more (e.g., 1, 2, 3, 4, 5, 6, 7, 8, 9, or 10 or more) 2′-OMe block and one or more (e.g., 1, 2, 3, 4, 5, 6, 7, 8, 9, or 10 or more) 2′-F block. In some embodiments, in a first domain there are one or more (e.g., 1, 2, 3, 4, 5, 6, 7, 8, 9, or 10 or more) 2′-F block and one or more (e.g., 1, 2, 3, 4, 5, 6, 7, 8, 9, or 10 or more) 2′-MOE block. In some embodiments, in a first domain there are one or more (e.g., 1, 2, 3, 4, 5, 6, 7, 8, 9, or 10 or more) 2′-OMe block and one or more (e.g., 1, 2, 3, 4, 5, 6, 7, 8, 9, or 10 or more) 2′-MOE block and one or more (e.g., 1, 2, 3, 4, 5, 6, 7, 8, 9, or 10) 2′-F block. In some embodiments, in an oligonucleotide or a portion thereof, e.g., a first domain, a second domain, etc., percentage of 2′-F modified sugars is about 20%-80%, 30-70%, 30%-60%, 30%-50%, 40%-60%, 20%, 30%, 40%, 50%, 60%, 70% or 80%, and percentage of 2′-OR modified sugars each of which is independently a 2′-OR modified sugar wherein R is optionally substituted C₁₋₆ aliphatic is about 20%-80%, 30-70%, 30%-60%, 30%-50%, 40%-60%, 20%, 30%, 40%, 50%, 60%, 70% or 80%. In some embodiments, in a first domain percentage of 2′-F modified sugars is about 20%-80%, 30-70%, 30%-60%, 30%-50%, 40%-60%, 20%, 30%, 40%, 50%, 60%, 70% or 80%, and percentage of 2′-OR modified sugars each of which is independently a 2′-OR modified sugar wherein R is optionally substituted C₁₋₆ aliphatic is about 20%-80%, 30-70%, 30%-60%, 30%-50%, 40%-60%, 20%, 30%, 40%, 50%, 60%, 70% or 80%. In some embodiments, the difference between the percentage of 2′-F modified sugars and the percentage of 2′-OR modified sugars each of which is independently a 2′-OR modified sugar wherein R is optionally substituted C₁₋₆ aliphatic is less than about 50%, 40%, 30%, 20%, or 10% (calculated by subtracting the smaller of the two percentages from the larger of the two percentages). In some embodiments, each 2′-OR modified sugar is independently a 2′-OMe or 2′-MOE modified sugar.

For example, in some embodiments, sugar of each of N₂, N₅, and N₆ is independently a 2′-F modified sugar, and sugar of each of N₃ and N₄ is independently a 2′-OR modified sugar wherein R is C₁₋₆ aliphatic or a bicyclic sugar. In some embodiments, sugar of each of N₂, N₅, and N₆ is independently a 2′-F modified sugar, and sugar of each of N₃ and N₄ is independently a 2′-OR modified sugar. In some embodiments, sugar of each of N₂, N₅, and N₆ is independently a 2′-F modified sugar, and sugar of each of N₃ and N₄ is independently a 2′-OMe or 2′-MOE modified sugar. In some embodiments, sugar of each of N₂, N₅, and N₆ is independently a 2′-F modified sugar, and sugar of each of N₃ and N₄ is independently a 2′-OMe modified sugar. In some embodiments, at least one sugar is a 2′-MOE modified sugar. In some embodiments, sugar of N₃ is a 2′-MOE modified sugar. In some embodiments, sugar of N₃ is a 2′-OMe modified sugar. In some embodiments, sugar of N₄ is a 2′-MOE modified sugar. In some embodiments, sugars of both N₃ and N₄ are 2′-MOE modified sugar. In some embodiments, N₂ forms a 2′-F block. In some embodiments, N₃ and N₄ forms a 2′-OMe block. In some embodiments, N₃ and N₄ forms a 2′-MOE block. In some embodiments, N₅, N₆ and/or N₇ form a 2′-F block. In some embodiments, oligonucleotides comprising modified sugars, e.g., 2′-F modified sugars, 2′-OMe modified sugars, 2′-MOE modified sugars, etc., at various positions can provide, among other things, high levels of adenosine editing. For example, 2′-MOE modified sugars can be incorporated at various positions to provide oligonucleotides capable of adenosine editing; in some embodiments, sugar of N₁ is a 2′-MOE modified sugar; in some embodiments, sugar of N₂ is a 2′-MOE modified sugar; in some embodiments, sugar of N₃ is a 2′-MOE modified sugar; in some embodiments, sugar of N₄ is a 2′-MOE modified sugar; in some embodiments, sugar of N₅ is a 2′-MOE modified sugar; in some embodiments, sugar of N₆ is a 2′-MOE modified sugar; in some embodiments, sugar of N₇ is a 2′-MOE modified sugar; in some embodiments, sugar of N₈ is a 2′-MOE modified sugar; in some embodiments, sugar of N₋₁ is a 2′-MOE modified sugar; in some embodiments, sugar of N₋₂ is a 2′-MOE modified sugar; in some embodiments, sugar of N₋₃ is a 2′-MOE modified sugar; in some embodiments, sugar of N₋₄ is a 2′-MOE modified sugar; in some embodiments, sugar of N₋₅ is a 2′-MOE modified sugar; in some embodiments, sugar of N₋₆ is a 2′-MOE modified sugar.

As described herein, various internucleotidic linkages may be utilized in oligonucleotides or portions thereof, e.g., first domains, second domains, etc. For example, various linkages may be utilized in first domains. In some embodiments, a first domain comprises one or more, e.g., 1, 2, 3, 4, 5, 6, 7, 8, 9, 10, 11, 12, 13, 14, 15, 16, 17, 18, 19, 20 or more natural phosphate linkages. In some embodiments, a first domain comprises one or more, e.g., 1, 2, 3, 4, 5, 6, 7, 8, 9, 10, 11, 12, 13, 14, 15, 16, 17, 18, 19, 20 or more, modified internucleotidic linkages. In some embodiments, a first domain comprises one or more natural phosphate linkages and one or more modified internucleotidic linkages. In some embodiments, one or more modified internucleotidic linkages are phosphorothioate internucleotidic linkages. In some embodiments, each phosphorothioate internucleotidic linkage is chirally controlled. In some embodiments, each phosphorothioate internucleotidic linkage in an oligonucleotide or a portion thereof, e.g., a first domain, a second domain, etc. is Sp. In some embodiments, each phosphorothioate internucleotidic linkage in an oligonucleotide is Sp. In some embodiments, one or more modified internucleotidic linkages are independently non-negatively charged internucleotidic linkage. In some embodiments, one or more modified internucleotidic linkages are independently non-negatively charged internucleotidic linkage. In some embodiments, one or more modified internucleotidic linkages are independently phosphoryl guanidine internucleotidic linkages. In some embodiments, each phosphoryl guanidine internucleotidic linkage is independently n001. In some embodiments, a first domain contains about 1-5, e.g., 1, 2, 3, 4, or 5 non-negatively charged internucleotidic linkages. In some embodiments, each of such non-negatively charged internucleotidic linkages are independently a phosphoryl guanidine internucleotidic linkage. In some embodiments, each of them is independently n001. In some embodiments, one or more of them are independently chirally controlled. In some embodiments, each of them is chirally controlled. In some embodiments, each of them is Rp n001. In some embodiments, one or more sugars that are 2′-OR modified sugars wherein R is optionally substituted C₁₋₆ aliphatic are boned to natural phosphate linkages. In some embodiments, one or more 2′-OMe sugars are bonded to natural phosphate linkages. In some embodiments, one or more 2′-MOE sugars are bonded to natural phosphate linkages. In some embodiments, one or more 2′-F modified sugars are bonded to natural phosphate linkages. In some embodiments, about or at least about 20%, 30%, 40%, 50%, 60%, 70%, 80% or 90% 2′-OR modified sugars wherein R is optionally substituted C₁₋₆ aliphatic in an oligonucleotide or a portion thereof, e.g., a first domain, a second domain, etc., are independently bonded to a natural phosphate linkage. In some embodiments, about or at least about 20%, 30%, 40%, 50%, 60%, 70%, 80% or 90% 2′-OMe modified sugars in an oligonucleotide or a portion thereof, e.g., a first domain, a second domain, etc., are independently bonded to a natural phosphate linkage. In some embodiments, about or at least about 20%, 30%, 40%, 50%, 60%, 70%, 80% or 90% 2′-MOE modified sugars in an oligonucleotide or a portion thereof, e.g., a first domain, a second domain, etc., are independently bonded to a natural phosphate linkage. In some embodiments, about or at least about 20%, 30%, 40%, 50%, 60%, 70%, 80% or 90% 2′-OR modified sugars wherein R is optionally substituted C₁₋₆ aliphatic in a first domain, a second domain are independently bonded to a natural phosphate linkage. In some embodiments, about or at least about 20%, 30%, 40%, 50%, 60%, 70%, 80% or 90% 2′-OMe modified sugars in a first domain are independently bonded to a natural phosphate linkage. In some embodiments, about or at least about 20%, 30%, 40%, 50%, 60%, 70%, 80% or 90% 2′-MOE modified sugars in a first domain are independently bonded to a natural phosphate linkage. In some embodiments, about or at least about 1-10, e.g., 1, 2, 3, 4, 5, 6, 7, 8, 9, or 10, 2′-OR modified sugars wherein R is optionally substituted C₁₋₆ aliphatic in a first domain, a second domain are independently bonded to a natural phosphate linkage. In some embodiments, about or at least about 1-10, e.g., 1, 2, 3, 4, 5, 6, 7, 8, 9, or 10, 2′-OMe modified sugars in a first domain are independently bonded to a natural phosphate linkage. In some embodiments, about or at least about 1-10, e.g., 1, 2, 3, 4, 5, 6, 7, 8, 9, or 10, 2′-MOE modified sugars in a first domain are independently bonded to a natural phosphate linkage. In some embodiments, one or more, e.g., 1, 2, 3, 4, 5, 6, 7, 8, 9, or 10 or more, natural phosphate linkage bonded to a 2′-F modified sugar are independently bonded to a 2′-OR modified sugar wherein R is optionally substituted C₁₋₆ aliphatic or a bicyclic sugar. In some embodiments, each natural phosphate linkage bonded to a 2′-F modified sugar is independently bonded to a 2′-OR modified sugar wherein R is optionally substituted C₁₋₆ aliphatic or a bicyclic sugar. In some embodiments, one or more, e.g., 1, 2, 3, 4, 5, 6, 7, 8, 9, or 10 or more, natural phosphate linkages bonded to a 2′-F modified sugar is independently bonded to a 2′-MOE modified sugar. In some embodiments, each natural phosphate linkage bonded to a 2′-F modified sugar is independently bonded to a 2′-MOE modified sugar.

Among other things, oligonucleotides comprising various blocks and patterns as described herein, e.g., 2′-F blocks, 2′-OMe blocks, 2′-MOE blocks, etc., and/or various internucleotidic linkages and patterns thereof as described herein, can provide improved pharmacodynamics, pharmacokinetics. and/or adenosine editing levels, etc., compared to comparable reference oligonucleotides, e.g., those previously reported in WO 2016/097212, WO 2017/220751, WO 2018/041973, WO 2018/134301A1, WO 2019/158475, WO 2019/219581, WO 2020/157008, WO 2020/165077, WO 2020/201406, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256. In some embodiments, a reference oligonucleotide is an oligonucleotide reported in WO 2021/071858, WO 2022/099159, and/or PCT/US2023/018742.

In some embodiments, N₋₂ contains a natural sugar. In some embodiments, sugar of N₋₂ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₋₁ and N₋₂ is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001. In some embodiments, N₋₁ is dI, and a linkage between N₋₁ and N₋₂ is a Sp phosphoryl guanidine internucleotidic linkage. In some embodiments, N₋₁ is dI, and a linkage between N₋₁ and N₋₂ is Sp n001.

In some embodiments, N₋₃ contains a natural sugar. In some embodiments, sugar of N₋₃ is a natural DNA sugar. In some embodiments, it is a natural RNA sugar. In some embodiments, it is a modified sugar. In some embodiments. it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₋₂ and N₋₃ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₋₄ contains a natural sugar. In some embodiments, sugar of N₋₄ is a natural DNA sugar. In some embodiments, it is a natural RNA sugar. In some embodiments, it is a modified sugar. In some embodiments. it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₋₃ and N₋₄ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₋₅ contains a natural sugar. In some embodiments, sugar of N₋₅ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₋₄ and N₋₅ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, N₋₆ contains a natural sugar. In some embodiments, sugar of N₋₆ is a natural DNA sugar. In some embodiments, it is a natural RNA sugar. In some embodiments, it is a modified sugar. In some embodiments, it is a 2′-F modified sugar. In some embodiments, it is a 2′-OR modified sugar, wherein R is C₁₋₆ aliphatic as described herein. In some embodiments, R is optionally substituted C₁₋₆ alkyl. In some embodiments, it is a 2′-OMe modified. In some embodiments, it is a 2′-MOE modified sugar.

In some embodiments, an internucleotidic linkage between N₋₅ and N₋₆ is a natural phosphate linkage. In some embodiments, it is a modified internucleotidic linkage. In some embodiments, it is a phosphorothioate internucleotidic linkage. In some embodiments, it is a non-negatively charged internucleotidic linkage. In some embodiments, it is a neutral internucleotidic linkage. In some embodiments, it is phosphoryl guanidine internucleotidic linkage. In some embodiments, it is n001. In some embodiments, it is Sp. In some embodiments, it is Rp. In some embodiments, it is a Sp phosphorothioate internucleotidic linkage. In some embodiments, it is Sp n001. In some embodiments, it is Rp n001.

In some embodiments, at least one sugar of N₋₁, N₋₂, N₋₃, N₋₄, N₋₅, and N₋₆ is a natural DNA sugar. In some embodiments, at least one sugar of N₋₂, N₋₃, N₋₄, N₋₅, and N₋₆ is a 2′-F modified sugar. In some embodiments, at least one sugar of N₋₂, N₋₃, N₋₄, N₋₅, and N₋₆ is a 2′-OR modified sugar wherein R is optionally substituted C₁₋₆ aliphatic. In some embodiments, at least one sugar of N₋₂, N₋₃, N₋₄, N₋₅, and N₋₆ is a 2′-OMe modified sugar. In some embodiments, at least one sugar of N₋₂, N₋₃, N₋₄, N₋₅, and N₋₆ is a 2′-MOE modified sugar. In some embodiments, at least one sugar of N₋₂, N₋₃, N₋₄, N₋₅, and N₋₆ is a bicyclic sugar, e.g., a LNA sugar, a cEt sugar, etc. In some embodiments, one sugar of N₋₂, N₋₃, N₋₄, N₋₅, and N₋₆ is a 2′-F modified sugar, and each of the other sugars are independently a 2′-OR modified sugar wherein R is C₁₋₆ aliphatic (e.g., a 2′-OMe modified sugar, a 2′-MOE modified sugar, etc.) or a bicyclic sugar as described herein. In some embodiments, one sugar of N₋₂, N₋₃, N₋₄, N₋₅, and N₋₆ is a 2′-F modified sugar, and each of the other sugars are independently a 2′-OR modified sugar wherein R is C₁₋₆ aliphatic. In some embodiments, one sugar of N₋₂, N₋₃, N₋₄, N₋₅, and N₋₆ is a 2′-F modified sugar, and each of the other sugars are independently a 2′-OMe or 2′-MOE modified sugar. In some embodiments, one sugar of N₋₂, N₋₃, N₋₄, N₋₅, and N₋₆ is a 2′-F modified sugar, and each of the other sugars are independently a 2′-OMe modified sugar. In some embodiments, sugar of N₋₃ a 2′-F modified sugar. In some embodiments, sugar of N₋₁ is a DNA sugar, sugar of N₋₃ is a 2′-F modified sugar, and sugar of each of N₋₂, N₋₄, N₋₅, and N₋₆ is independently a 2′-OR modified sugar wherein R is optionally substituted C₁₋₆ aliphatic or a bicyclic sugar (e.g., a LNA sugar, an ENA sugar, etc.) as described herein. In some embodiments, sugar of N₋₁ is a DNA sugar, sugar of N₋₃ is a 2′-F modified sugar, and sugar of each of N₋₂, N₋₄, N₋₅, and N₋₆ is independently a 2′-OR modified sugar wherein R is optionally substituted C₁₋₆ aliphatic. In some embodiments, sugar of N₋₁ is a DNA sugar, sugar of N₋₃ is a 2′-F modified sugar, and sugar of each of N₋₂, N₋₄, N₋₅, and N₋₆ is independently a 2′-OMe or 2′-MOE modified sugar. In some embodiments, sugar of N₋₁ is a DNA sugar, sugar of N₋₃ is a 2′-F modified sugar, and sugar of each of N₋₂, N₋₄, N₋₅, and N₋₆ is independently a 2′-OMe modified sugar. In some embodiments, N₋₂ forms a 2′-OMe block. In some embodiments, N₋₃ forms a 2′-F block. In some embodiments, N₋₄, N₋₅, and N₋₆ forms a 2′-OMe block.

In some embodiments, at least one of N₋₂, N₋₃, N₋₄, N₋₅, and N₋₆ is bonded to a natural phosphate linkage. In some embodiments, a linkage between N₋₂ and N₋₃ is a natural phosphate linkage. In some embodiments, N₋₂ is bonded to a non-negatively charged internucleotidic linkage. In some embodiments, at least one of N₋₃, N₋₄, N₋₅, and N₋₆ is bonded to a non-negatively charged internucleotidic linkage. In some embodiments, a linkage between N₋₅ and N₋₆ is a non-negatively charged internucleotidic linkage. In some embodiments, at least one of N₋₃, N₋₄, N₋₅, and N₋₆ is bonded to a phosphorothioate internucleotidic linkage. In some embodiments, each of N₋₃, N₋₄ and N₋₅ is independently bonded to a phosphorothioate internucleotidic linkage. In some embodiments, a non-negatively charged internucleotidic linkage is a neutral internucleotidic linkage. In some embodiments, a non-negatively charged internucleotidic linkage is a phosphoryl guanidine internucleotidic linkage. In some embodiments, a non-negatively charged internucleotidic linkage is n001. In some embodiments, it is Rp. In some embodiments, it is Sp. In some embodiments, a phosphorothioate internucleotidic linkage is Rp. In some embodiments, a phosphorothioate internucleotidic linkage is Sp. In some embodiments, each phosphorothioate internucleotidic linkage is Sp. In some embodiments, a linkage between N₋₂ and v-3 is a natural phosphate linkage, a linkage between N₋₃ and N₋₄ is a Sp phosphorothioate internucleotidic linkage, a linkage between N₋₄ and N₋₅ is a Sp phosphorothioate internucleotidic linkage, and a linkage between N₋₅ and N₋₆ is a Rp non-negatively charged internucleotidic linkage (e.g., a Rp phosphoryl guanidine internucleotidic linkage such as Rp n001). In some embodiments, a natural phosphate linkage is bonded to at least one modified sugar. In some embodiments, a natural phosphate linkage is bonded to at least one 2′-OR modified sugar wherein R is C₁₋₆ aliphatic or a bicyclic sugar. In some embodiments, a natural phosphate linkage is bonded to a 2′-OMe modified sugar. In some embodiments, a natural phosphate linkage is bonded to a 2′-MOE modified sugar. In some embodiments, both sugars bonded to a natural phosphate linkage is independently a modified sugar as described herein.

In some embodiments, an oligonucleotide comprises a first domain as described herein (e.g., a first domain in which multiple or a majority of or all of sugars are 2′-F modified sugars) and a second domain as described herein (e.g., a second domain in which multiple or a majority of or all of sugars are non-2′-F modified sugars (e.g., 2′-OMe modified sugars)). In some embodiments, a first domain is at the 5′ side of a second domain. In some embodiments, a first domain is at the 3′ side of a second domain. In some embodiments, when a first domain is at the 3′ side of a second domain, there is at least 1, 2, 3, 4, 5, 6, 7, 8, 9, or 10 or more (e.g., 1-20, 2-20, 3-20, 4-20, 5-20, 6-20, 7-20, 7-11, etc.) 5′-side nucleosides of a nucleoside opposite to a target adenosine. In some embodiments, there are at least 3. In some embodiments, there are at least 4. In some embodiments, there are at least 5. In some embodiments, there are at least 6. In some embodiments, there are at least 7. In some embodiments, there are at least 8. In some embodiments, there are at least 9. In some embodiments, there are at least 10. In some embodiments, there are 3. In some embodiments, there are 4. In some embodiments, there are 5. In some embodiments, there are 6. In some embodiments, there are 7. In some embodiments, there are 8. In some embodiments, there are 9. In some embodiments, there are 10. In some embodiments, there are 11. In some embodiments, there are 7-11. In some embodiments, there are 9-11. In some embodiments, there are 10 or 11. In some embodiments, additionally or alternatively, there are at least 15, 16, 17, 18, 19, 20 or more (e.g., 15-30, 16-30, 17-30, 18-30, 18-25, 18-22, etc.) 5′-side nucleosides of a nucleoside opposite to a target adenosine. In some embodiments, there are at least 15. In some embodiments, there are at least 16. In some embodiments, there are at least 17. In some embodiments, there are at least 18. In some embodiments, as described above, there are at least about 5 (e.g., 5-50, 5-40, 5-30, 5-20, 5-10, 5-9, 5, 6, 7, 8, 9, or 10, etc.) 3′-side nucleosides and at least about 15 (e.g., 15-50, 15-40, 15-30, 15-20, 20-30, 20-25, 15, 16, 17, 18, 19, 20, 21, 22, 23, 24, 25, 26, 27, 28, 29 or 30, etc.) 5′-side nucleosides. In some embodiments, independently about 1-10 (e.g., 2-10, 3-10, 3-5, 1, 2, 3, 4, 5, 6, 7, 8, 9, or 10) bicyclic or 2′-OR modified sugars are independently on the 5′-, or 3′-, or both sides of an editing region (e.g., N₁N₀N_{‘1}), wherein R is optionally substituted C₁₋₆ aliphatic. In some embodiments, independently about 1-10 (e.g., 2-10, 3-10, 3-5, 1, 2, 3, 4, 5, 6, 7, 8, 9, or 10) 2′-OR modified sugars are independently on the 5′-, or 3′-, or both sides of an editing region, wherein R is optionally substituted C₁₋₆ aliphatic. In some embodiments, independently about 1-10 (e.g., 2-10, 3-10, 3-5, 1, 2, 3, 4, 5, 6, 7, 8, 9, or 10) 2′-OMe modified sugars are independently on the 5′-, or 3′-, or both sides of an editing region. In some embodiments, they are on the 5′ side. In some embodiments, they are on the 3′ sides. In some embodiments, they are on both sides. In some embodiments, it is beneficial that surrounding an editing region, e.g., N₁N₀N₋₁, there are bicyclic or 2′-OR modified sugars, e.g., independently about 1-10 (e.g., 2-10, 3-10, 3-5, 1, 2, 3, 4, 5, 6, 7, 8, 9, or 10), on both sides, wherein R is optionally substituted C₁₋₆ aliphatic. In some embodiments, it is beneficial that surrounding an editing region, e.g., N₁N₀N₋₁, there are 2′-OR modified sugars, e.g., independently about 1-10 (e.g., 2-10, 3-10, 3-5, 1, 2, 3, 4, 5, 6, 7, 8, 9, or 10), on both sides, wherein R is optionally substituted C₁₋₆ aliphatic. In some embodiments, on each side there are at least 2. In some embodiments, each 2′-OR modified sugar is a 2′-OMe modified sugar. In some embodiments, each 2′-OR modified sugar is a 2′-MOE modified sugar.

One of the many advantages of provided technologies is that much shorter oligonucleotides compared to traditional technologies of others can provide comparable or higher levels of adenosine editing. Those skilled in the art reading the present disclosure will appreciate that longer oligonucleotides (e.g., extending 5′ side, 3′ side or both sides of a target adenosine) incorporating one or more structural elements (e.g., sugar modifications, nucleobase modifications, internucleotidic linkage modifications, stereochemistry, and/or patterns thereof) of oligonucleotides of the present disclosure may also be useful, e.g., for various uses described herein including adenine editing and prevention and/or treatment of conditions, disorders or diseases which can benefit editing of target adenosines.

In some embodiments, ADAR1 p150 may tolerate variations of lengths of 5′-sides and/or 3′-sides and/or positioning of nucleosides opposite to target adenosines more than ADAR1 p110. In some embodiments, the present disclosure provides particularly useful lengths of 5′-sides and/or 3′-sides and/or positioning of nucleosides opposite to target adenosines for editing (e.g., by ADAR1 p110 and/or ADAR1 p150). In some embodiments, certain useful lengths of 5′-sides and/or 3′-sides and/or positioning of nucleosides are useful for editing in cells expressing ADAR1, e.g., ADAR1 p110 and/or p150.

In some embodiments, each phosphorothioate bonded to a nucleoside opposite to atarget adenosine is independently a phosphorothioate internucleotidic linkage. In some embodiments, the internucleotidic linkage between N₀ and N₋₁ is a Rp phosphorothioate internucleotidic linkage. In some embodiments, the internucleotidic linkage between N₋₁ and N₋₂ is a Rp phosphorothioate internucleotidic linkage.

In some embodiments, the present disclosure provides oligonucleotides comprising editing regions that can provide high editing efficiency. In some embodiments, a provided editing region is or comprises 5′-N₁N₀N₋₁-3′ as described herein.

In some embodiments, the present disclosure provides oligonucleotides comprising 5′-N₁N₀N₋₁-3′ as described herein.

In some embodiments, N₀ is as described herein. In some embodiments, N₀ comprises a sugar and a nucleobase as described herein. In some embodiments, N₀ has a natural DNA sugar. In some embodiments, N₀ has a natural RNA sugar. In some embodiments, N₀ has a modified sugar, e.g., a 2′-F modified sugar. In some embodiments, sugar of a nucleobase opposite to a target adenosine. or N₀, is arabinofuranose. In some embodiments, sugar of a nucleobase opposite to a target adenosine, or N₀, is

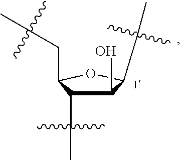

wherein C1′ bonds to a nucleobase as described herein. In some embodiments, N₀ has a natural nucleobase. In some embodiments, nucleobase of N₀ is C. In some embodiments, nucleobase of N₀ is b001A. In some embodiments, N₀ is deoxycytidine. In some embodiments, N₀ is cytidine. In some embodiments, N₀ is 2′-F C (wherein 2′-OH of cytidine is replaced with —F). In some embodiments, N₀ is b001A. In some embodiments, N₀ is Csm15. In some embodiments, N₀ is b001rA. In some embodiments, N₀ is b008U. In some embodiments, nucleobase of N₀ is not T or U. In some embodiments, nucleobase of N₀ is not T. In some embodiments, nucleobase of N₀ is not U. In some embodiments, N₀ is not a match to A.

In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A.

In some embodiments, nucleobase of N₁ is T, and nucleobase of N₋₁ is hypoxanthine. In some embodiments, N₁ is thymidine, and N₋₁ is deoxyinosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A.

In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A.

In some embodiments, nucleobase of N₁ is U, and nucleobase of N₋₁ is guanine. In some embodiments, N₁ is 2′-F U, and N₋₁ is deoxyguanosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A. In some embodiments, there are 6 or at least 6 nucleosides to the 3′ side of N₀(e.g., when there are 6, N₋₁ to N₋₆).

In some embodiments, nucleobase of N₁ is C, and nucleobase of N₋₁ is guanine. In some embodiments, N₁ is 2′-F C, and N₋₁ is deoxyguanosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A. In some embodiments, there are 6 or at least 6 nucleosides to the 3′ side of N₀(e.g., when there are 6, N₋₁ to N₋₆).

In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is U, and nucleobase of N₋₁ is G. In some embodiments, N₁ is 2′-F U, and N₋₁ is dG. In some embodiments, nucleobase of N₁ is C, and nucleobase of N₋₁ is G. In some embodiments, N₁ is 2′-F C, and N₋₁ is dG. In some embodiments, nucleobase of N₁ is G. and nucleobase of N₋₁ is hypoxanthine. In some embodiments, N₁ is 2′-F G, and N₋₁ is deoxyinosine. In some embodiments, nucleobase of N₀ is as described herein such as cytosine, b001A, b008U, etc. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAA -3′ for editing a target adenosine A.

In some embodiments, nucleobase of N₁ is U, and nucleobase of N₋₁ is T. In some embodiments, N₁ is 2′-F U, and N₋₁ is dT. In some embodiments, nucleobase of N₁ is C, and nucleobase of N₋₁ is thymine. In some embodiments, N₁ is 2′-F C, and N₋₁ is dT. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is U, and nucleobase of N₋₁ is G. In some embodiments, N₁ is 2′-F U, and N₋₁ is dG. In some embodiments, nucleobase of N₁ is C, and nucleobase of N₋₁ is hypoxanthine. In some embodiments, N₁ is 2′-F U, and N₋₁ is deoxyinosine. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-AAA -3′ for editing a target adenosine A.

In some embodiments, nucleobase of N₁ is A, and nucleobase of N₋₁ is T. In some embodiments, N₁ is 2′-F A, and N₋₁ is dT. In some embodiments, nucleobase of N₁ is G, and nucleobase of N₋₁ is T. In some embodiments, N₁ is 2′-F G, and N₋₁ is dT. In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is A, and nucleobase of N₋₁ is G. In some embodiments, N₁ is 2′-F A, and N₋₁ is dG. In some embodiments, nucleobase of N₁ is A, and nucleobase of N₋₁ is C. In some embodiments, N₁ is 2′-F A, and N₋₁ is dC. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, No is Csm15. In some embodiments, N₀ is b001rA. In some embodiments, N₀ is b008U. In some embodiments, such 5′-N₁N₀N₋₁-3′are particularly useful for targeting RNA comprising 5′-AAU -3′ for editing a target adenosine A.

In some embodiments, nucleobase of N₁ is U, and nucleobase of N₋₁ is T. In some embodiments, N₁ is 2′-F U, and N₋₁ is dT. In some embodiments, nucleobase of N₁ is C, and nucleobase of N₋₁ is T. In some embodiments, N₁ is 2′-F C, and N₋₁ is dT. In some embodiments, nucleobase of N₁ is C, and nucleobase of N₋₁ is A. In some embodiments, N₁ is 2′-F C, and N₋₁ is dA. In some embodiments, nucleobase of N₁ is C, and nucleobase of N₋₁ is C. In some embodiments, N₁ is 2′-F C, and N₋₁ is dC. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is C, and nucleobase of N₋₁ is G. In some embodiments, N₁ is 2′-F C, and N₋₁ is dG. In some embodiments, nucleobase of N₁ is U, and nucleobase of N₋₁ is G. In some embodiments, N₁ is 2′-F U, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-AAG -3′ for editing a target adenosine A.

In some embodiments, nucleobase of N₁ is G, and nucleobase of N₋₁ is T. In some embodiments, N₁ is 2′-F G, and N₋₁ is dT. In some embodiments, nucleobase of N₁ is G, and nucleobase of N₋₁ is A. In some embodiments, N₁ is 2′-F G, and N₋₁ is dA. In some embodiments, nucleobase of N₁ is G, and nucleobase of N⁻¹ is C. In some embodiments, N₁ is 2′-F G, and N₋₁ is dC. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is G, and nucleobase of N₋₁ is G. In some embodiments, N₁ is 2′-F G, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-AAC -3′ for editing a target adenosine A.

In some embodiments, nucleobase of N₁ is U, and nucleobase of N₋₁ is A. In some embodiments, N₁ is 2′-F U, and N₋₁ is dA. In some embodiments, nucleobase of N₁ is C, and nucleobase of N₋₁ is A. In some embodiments, N, is 2′-F C, and N₋₁ is dA. In some embodiments, nucleobase of N₁ is A, and nucleobase of N₋₁ is A. In some embodiments, N₁ is 2′-F A, and N₋₁ is dA. In some embodiments, nucleobase of N₁ is G, and nucleobase of N₋₁ is A. In some embodiments, N₁ is 2′-F G, and N₋₁ is dA. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, No is b008U. In some embodiments, such 5′-N₁N₀N₋₁-3′are particularly useful for targeting RNA comprising 5′-UAA -3′ for editing a target adenosine A.

In some embodiments, nucleobase of N₁ is A, and nucleobase of N₋₁ is A. In some embodiments, N₁ is 2′-F A, and N₋₁ is dA. In some embodiments, nucleobase of N₁ is G, and nucleobase of N₋₁ is A. In some embodiments, N₁ is 2′-F G, and N₋₁ is dA. In some embodiments, nucleobase of N₁ is C, and nucleobase of N₋₁ is A. In some embodiments, N₁ is 2′-F C, and N₋₁ is dA. In some embodiments, nucleobase of N₁ is U, and nucleobase of N₋₁ is A. In some embodiments, N₁ is 2′-F U, and N₋₁ is dA. In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is G, and nucleobase of N₋₁ is hypoxanthine. In some embodiments, N₁ is 2′-F G, and N₋₁ is deoxyinosine. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, No is b008U. In some embodiments, such 5′-N₁N₀N₋₁-3′are particularly useful for targeting RNA comprising 5′-UAU -3′ for editing a target adenosine A.

In some embodiments, nucleobase of N₁ is C, and nucleobase of N₋₁ is A. In some embodiments, N₁ is 2′-F C. and N₋₁ is dA. In some embodiments, nucleobase of N₁ is U, and nucleobase of N₋₁ is A. In some embodiments, N₁ is 2′-F U, and N₋₁ is dA. In some embodiments, nucleobase of N₁ is C, and nucleobase of N₋₁ is T. In some embodiments, N₁ is 2′-F C, and N₋₁ is dT. In some embodiments, nucleobase of N₁ is A, and nucleobase of N₋₁ is A. In some embodiments, N₁ is 2′-F A, and N₋₁ is dA. In some embodiments, nucleobase of N₁ is G, and nucleobase of N₋₁ is A. In some embodiments, N₁ is 2′-F G, and N₋₁ is dA. In some embodiments, nucleobase of N₁ is C, and nucleobase of N₋₁ is C. In some embodiments, Ni is 2′-F C, and N₋₁ is dC. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is C, and nucleobase of N₋₁ is G. In some embodiments, N₁ is 2′-F C, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-UAG -3′ for editing a target adenosine A.

In some embodiments, nucleobase of N₁ is G, and nucleobase of N₋₁ is T. In some embodiments, N₁ is 2′-F G, and N₋₁ is dT. In some embodiments, nucleobase of N₁ is U, and nucleobase of N₋₁ is A. In some embodiments, N₁ is 2′-F U, and N₋₁ is dA. In some embodiments, nucleobase of N₁ is A, and nucleobase of N₋₁ is A. In some embodiments, N₁ is 2′-F A, and N₋₁ is dA. In some embodiments, nucleobase of N₁ is C, and nucleobase of N₋₁ is A. In some embodiments, N₁ is 2′-F C, and N₋₁ is dA. In some embodiments, nucleobase of N₁ is G, and nucleobase of N₋₁ is A. In some embodiments, N₁ is 2′-F G, and N₋₁ is dA. In some embodiments, nucleobase of N₁ is G, and nucleobase of N₋₁ is C. In some embodiments, N₁ is 2′-F G, and N₋₁ is dC. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is G, and nucleobase of N₋₁ is G. In some embodiments, N₁ is 2′-F G, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-UAC -3′ for editing a target adenosine A.

In some embodiments, nucleobase of N₁ is C, and nucleobase of N₋₁ is C. In some embodiments, N₁ is 2′-F C, and N₋₁ is dC. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is U, and nucleobase of N₋₁ is G. In some embodiments, N₁ is 2′-F U, and N₋₁ is dG. In some embodiments, nucleobase of N₁ is C, and nucleobase of N₋₁ is G. In some embodiments, N₁ is 2′-F C, and N₋₁ is dG. In some embodiments, N₀ is as described herein such as deoxycytidine. b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-GAA -3′ for editing a target adenosine A.

In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is A, and nucleobase of N₋₁ is G. In some embodiments, N₁ is 2′-F A, and N₋₁ is dG. In some embodiments, nucleobase of N₁ is G, and nucleobase of N₋₁ is G. In some embodiments, N₁ is 2′-F G, and N₋₁ is dG. In some embodiments, nucleobase of N₁ is A, and nucleobase of N₋₁ is A. In some embodiments, N₁ is 2′-F A, and N₋₁ is dA. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-GAU -3′ for editing a target adenosine A.

In some embodiments, nucleobase of N₁ is C, and nucleobase of N₋₁ is T. In some embodiments, N₁ is 2′-F C, and N₋₁ is dT. In some embodiments, nucleobase of N₁ is C, and nucleobase of N₋₁ is A. In some embodiments, N₁ is 2′-F C, and N₋₁ is dA. In some embodiments, nucleobase of N₁ is C, and nucleobase of N₋₁ is C. In some embodiments, N₁ is 2′-F C, and N₋₁ is dC. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is U, and nucleobase of N₋₁ is G. In some embodiments, N₁ is 2′-F U, and N₋₁ is dG. In some embodiments, nucleobase of N₁ is C, and nucleobase of N₋₁ is G. In some embodiments, N₁ is 2′-F C, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-GAG -3′ for editing a target adenosine A.

In some embodiments, nucleobase of N₁ is G, and nucleobase of N₋₁ is A. In some embodiments, N₁ is 2′-F G, and N₋₁ is dA. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is G, and nucleobase of N₋₁ is G. In some embodiments, N₁ is 2′-F G, and N₋₁ is dG. In some embodiments, nucleobase of N₁ is G, and nucleobase of N₋₁ is T. In some embodiments, N₁ is 2′-F G, and N₋₁ is dT. In some embodiments, nucleobase of N₁ is G, and nucleobase of N₋₁ is C. In some embodiments, N₁ is 2′-F G, and N₋₁ is dC. In some embodiments, N₀ is as described herein such as deoxycytidine, b001A, Csm15, b001rA, b008U, etc. In some embodiments, N₀ is deoxycytidine. In some embodiments. No is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-GAC -3′ for editing a target adenosine A.

In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is A, and nucleobase of N₋₁ is G. In some embodiments, N₁ is 2′-F A, and N₋₁ is dG. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAU -3′ for editing a target adenosine A.

In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is U, and nucleobase of N₋₁ is G. In some embodiments, N₁ is 2′-F U, and N₋₁ is dG. In some embodiments, nucleobase of N₁ is C, and nucleobase of N₋₁ is G. In some embodiments, N₁ is 2′-F C, and N₋₁ is dG. In some embodiments, nucleobase of N₁ is G, and nucleobase of N₋₁ is G. In some embodiments, N₁ is 2′-F G, and N₋₁ is dG. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAG -3′ for editing a target adenosine A.

In some embodiments, nucleobase of N₁ is A, and nucleobase of N₋₁ is hypoxanthine. In some embodiments, N₁ is 2′-F A, and N₋₁ is deoxyinosine. In some embodiments, nucleobase of N₁ is G, and nucleobase of N₋₁ is hypoxanthine. In some embodiments, N₁ is 2′-F G, and N₋₁ is deoxyinosine. In some embodiments, nucleobase of N₁ is G, and nucleobase of N₋₁ is G. In some embodiments, N₁ is 2′-F G, and N₋₁ is dG. In some embodiments, nucleobase of N₁ is U, and nucleobase of N₋₁ is hypoxanthine. In some embodiments, N₁ is 2′-F U, and N₋₁ is deoxyinosine. In some embodiments, N₀ is as described herein such as deoxycytidine, b001A, Csm15, b001rA, b008U, etc. In some embodiments, N₀ is deoxycytidine. In some embodiments, N₀ is b001A. In some embodiments, N₀ is Csm15. In some embodiments, N₀ is b001rA. In some embodiments, N₀ is b008U. In some embodiments, such 5′-N₁N₀N₋₁-3′are particularly useful for targeting RNA comprising 5′-CAC -3′ for editing a target adenosine A.

In some embodiments, nucleobase of N₁ is U, and nucleobase of N₋₁ is A. In some embodiments, N₁ is 2′-F U, and N₋₁ is dA. In some embodiments, nucleobase of N₁ is C, and nucleobase of N₋₁ is A. In some embodiments, N₁ is 2′-F C, and N₋₁ is dA. In some embodiments, nucleobase of N₁ is G, and nucleobase of N₋₁ is A. In some embodiments, N₁ is 2′-F G, and N₋₁ is dA. In some embodiments, nucleobase of N₁ is C, and nucleobase of N₋₁ is hypoxanthine. In some embodiments, N₁ is 2′-F C, and N₋₁ is deoxyinosine. In some embodiments, nucleobase of N₁ is U, and nucleobase of N₋₁ is hypoxanthine. In some embodiments, N₁ is 2′-F U, and N₋₁ is deoxyinosine. In some embodiments, nucleobase of N₁ is A, and nucleobase of N₋₁ is A. In some embodiments, N₁ is 2′-F A, and N₋₁ is dA. In some embodiments, N₀ is b001rA. In some embodiments, N₀ is b008U. In some embodiments, such 5′-N₁N₀N₋₁-3′are particularly useful for targeting RNA comprising 5′-UAG -3′ for editing a target adenosine A.

In some embodiments, nucleobase U may be replaced with T without lowering editing levels. In some embodiments, nucleobase U may be replaced with T to increase editing levels. In some embodiments, 2′-F U may be replaced with thymidine. In some embodiments, N₁ is thymidine. In some embodiments, N₁ is thymidine, N₀ is as described herein, e.g., b001A, b008U, etc. In some embodiments, N₁ is thymidine, N₀ is as described herein, e.g., b001A, b008U, etc., and N₋₁ is I.

In some embodiments, when being aligned to a target sequence and/or hybridized to a target nucleic acid, N₀ is a wobble or mismatch to A. In some embodiments, N₁ is not a match to its opposite nucleobase. In some embodiments, N₋₁ is not a match to its opposite nucleobase. In some embodiments, two of N₋₁, N₀ and N₁ are independently not a match to its opposite nucleobase. In some embodiments, N₀ and N₁ are independently not a match to its opposite nucleobase. In some embodiments, N₀ and N₋₁ are independently not a match to its opposite nucleobase. In some embodiments, when it is not a match, it is a wobble. In some embodiments, when it is not a match, it is a mismatch. In some embodiments, nucleobase of N₁ is C and its opposite nucleobase is A. In some embodiments, more nucleosides to the 3′ side of N₀(e.g., 6 or more) may tolerate more mismatches/wobbles of 5′-N₁N₀N₋₁-3′.

In some embodiments, each internucleotidic linkage bonded to No is independently Sp phosphorothioate internucleotidic linkages. In some embodiments, each internucleotidic linkage bonded to Ni is independently Sp phosphorothioate internucleotidic linkages. In some embodiments, an internucleotidic linkage bonded to N₋₁ is a non-negatively charged internucleotidic linkage. In some embodiments, an internucleotidic linkage bonded to N₋₁ is a neutral internucleotidic linkage. In some embodiments, an internucleotidic linkage bonded to N₋₁ is a phosphoryl guanidine internucleotidic linkage. In some embodiments, an internucleotidic linkage bonded to N₋₁ is n001. In some embodiments, a phosphoryl guanidine internucleotidic linkage, e.g., n001, bonded to N₋₁ (e.g., to its position 3′) is chirally controlled and is Rp. In some embodiments, a phosphoryl guanidine internucleotidic linkage, e.g., n001, bonded to N₋₁ (e.g., to its position 3′) is chirally controlled and is Sp (e.g., in some embodiments, when N₋₁ is dI).

In some embodiments, an oligonucleotide may comprise more than one editing region, each independently as described herein, for targeted editing of more than one target adenosines. In some embodiments, an oligonucleotide may comprise more than one 5′-N₁N₀N₋₁-3′. In some embodiments, an oligonucleotide may comprise more than one 5′-N₁N₀N₋₁-3′, each independently as described herein, for targeted editing of more than one target adenosines. For example, in some embodiments, a first domain comprises an editing region targeting a first target adenosine. In some embodiments, a second domain comprises an editing region targeting a second target adenosine. In some embodiments, each editing region is independently or independently comprises 5′-N₁N₀N₋₁-3′ as described herein. In some embodiments, N₀, N₁, and N₋₁ of each 5′-N₁N₀N₋₁-3′ are independently as described herein. In some embodiments, flanking sequences of each 5′-N₁N₀N₋₁-3′ are independently as described herein. In some embodiments, a first domain is or comprises a second domain. In some embodiments, a first domain is or comprises a second subdomain as described herein. In some embodiments, a first domain is or comprises a first subdomain, second subdomain and/or a third subdomain, each of which is independently as described herein. In some embodiments, an oligonucleotide is capable of editing a first target adenosine. In some embodiments, an oligonucleotide is capable of editing a second target adenosine. In some embodiments, an oligonucleotide is capable of a first target adenosine and a second target adenosine on the same nucleic acid, e.g., a mRNA. In some embodiments, a first target adenosine and a second target adenosine are in a same sequence, e.g., of a transcript such as mRNA. In some embodiments, they are separated by a gap as described herein, e.g., a gap about or at least about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 500, 1000, 2-100,000, 2-50,000, 2-10,000, 2-5,000, 3-100,000, 3-50,000, 3-10,000, 3-5,000, 4-100,000, 4-50,000, 4-10,000, 4-5,000, 5-100,000, 5-50,000, 5-10,000, 5-5,000, 10-100,000, 10-50,000, 10-10,000, 10-5,000, 20-100,000, 20-50,000, 20-10,000, 20-5,000, 50-100,000, 50-50,000, 50-10,000, 50-5,000, 100-100,000, 100-50,000, 100-10,000, or 100-5,000, nucleobases.

### Linker

In some embodiments, a first domain is directly connected to a second domain. In some embodiments, a first domain is connected to a second domain through a linker.

In some embodiments, a first domain is connected to a second domain through L^{FS}, which is a covalent bond, or a bivalent, optionally substituted, linear or branched group selected from a C₁₋₅₀ (e.g., C₁₋₁₀, C₁₋₂₀, C₁₋₃₀, C₁₋₄₀, or about C₁₀, C₂₀, C₃₀, C₄₀, C₁₋₅₀, etc.) aliphatic group and a C₁₋₅₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, —Cy—, —O—, —S—, —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, a nucleotide, an amino acid, and —[C(R′)₂C(R′)₂O]n-, wherein n is 1-50, and one or more nitrogen or carbon atoms are optionally and independently replaced with Cy^{L}, wherein each variable is independently as described herein.

In some embodiments, a linker is or comprises of [SpC3], [oC4o], [oC6o], [Sp9], or [Sp12]. In some embodiments, a linker is or comprises [SpC3]. In some embodiments, a linker is or comprises of [oC4o]. In some embodiments, a linker is or comprises [oC6o]. In some embodiments, a linker is or comprises of [Sp9]. In some embodiments, a linker is or comprises [Sp12]. In some embodiments, a linker is or comprises —(CH₂)ₙ—, wherein n is 1-50. In some embodiments, a linker is or comprises —O(CH₂)ₙO—, wherein n is 1-50. In some embodiments, a linker is or comprises —(CH₂CH₂O)ₙCH₂CH₂—, wherein n is 1-50. In some embodiments, a linker is or comprises —O(CH₂CH₂O)ₙCH₂CH₂O—, wherein n is 1-50. In some embodiments, n is about 1-40, 1-30, 1-25, 1-20, 1-15, 1-10, 1, 2, 3, 4, 5, 6, 7, 8, 9, 10, etc. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, a linker is or comprises

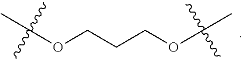 [SMILES: *OCCCO*]

In some embodiments, a linker is or comprises

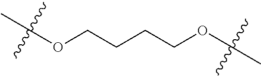 [SMILES: *OCCCCO*]

In some embodiments, a linker is or comprises

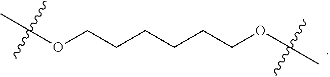 [SMILES: *OCCCCCCO*]

In some embodiments, a linker is or comprises

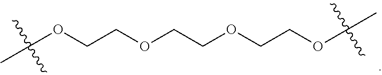 [SMILES: *OCCOCCOCCO*]

In some embodiments, a linker is or comprises

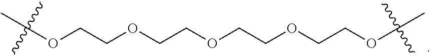 [SMILES: *OCCOCCOCCOCCO*]

In some embodiments, a linker is or comprises a nucleoside. In some embodiments, a linker is or comprises an oligonucleotide (“linker oligonucleotide”). In some embodiments, length of a linker oligonucleotide is about or at least about 2-100, 2-90, 2-80, 2-70, 2-60, 2-50, 2-40, 2-30, 2-20, 2-10, 2, 3, 4, 5, 6, 7, 8, 9, 10, 11, 12, 13, 14, 15, 16, 17, 18, 19, or 20 nucleobases. In some embodiments, complementarity of a linker oligonucleotide to a gap in a target nucleic acid is no more than about 90%, 85%, 80%, 75%, 70%, 65%, 60%, 50%, 40%, 35%, 30%, 25%, 20%, 10%, or 5%. In some embodiments, it is no more than about 85%. In some embodiments, it is no more than about 80%. In some embodiments, it is no more than about 75%. In some embodiments, it is no more than about 70%. In some embodiments, it is no more than about 65%. In some embodiments, it is no more than about 60%. In some embodiments, it is no more than about 55%. In some embodiments, it is no more than about 50%. In some embodiments, it is no more than about 40%. In some embodiments, it is no more than about 35%. In some embodiments, it is no more than about 30%. In some embodiments, it is no more than about 25%. In some embodiments, it is no more than about 20%. In some embodiments, it is no more than about 10%. In some embodiments, it is no more than about 5%. In some embodiments, a linker is or comprises polyvinylether, polyethylene, polypropylene, polyethylene glycol (PEG), polypropylene glycol (PEG), polyvinyl alcohol (PVA), polyglycolide (PGA), polylactide (PLA), polycaprolactone (PCL), or copolymers thereof In some embodiments, a linker is, comprises or consists of one or more units each independently selected from

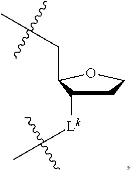

—(CH₂)ₙ-L^{k}-, and —(CH₂CH₂O)ₙCH₂CH₂-L^{k}-, wherein each n is independently about 1-100, 1-50, 1-40, 1-30, 1-20, 1-10, 1-5, 2-100, 2-50, 2-40, 2-30, 2-20, 2-10, 2-5, 3-100, 3-50, 3-40, 3-30, 3-20, 3-10, 3-5, 1, 2, 3, 4, 5, 6, 7, 8, 9, 10, 11, 12, 13, 14, 15, 16, 17, 18, 19, or 20, and each L^{k} independently has the structure of internucleotidic linkage. In some embodiments, a linker comprises one or more units each independently selected from

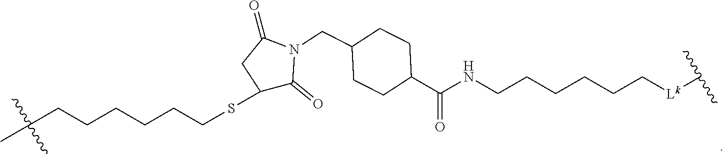

In some embodiments, each L^{k}independently has the structure of a PO, PS or PN linkage. In some embodiments, each L^{k} independently has the structure of a PO, PS or phosphoryl guanidine linkage. In some embodiments, at least one L^{k} is has the structure of a natural phosphate linkage. In some embodiments, at least one L^{k} has the structure of phosphorothioate internucleotidic linkage. In some embodiments, at least one L^{k} has the structure of a PN linkage. In some embodiments, at least one L^{k} has the structure of a phosphoryl guanidine linkage. In some embodiments, at least one L^{k} has the structure of n001. In some embodiments, each L^{k} independently has the structure of a natural phosphate linkage, a phosphorothioate internucleotidic linkage or a phosphoryl guanidine internucleotidic linkage. In some embodiments, each L^{k} independently has the structure of a natural phosphate linkage, a phosphorothioate internucleotidic linkage or n001. In some embodiments, a linker is, comprises or consists of one or more units each independently selected from

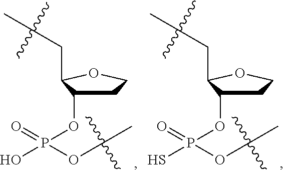

—(CH₂)ₙOP(O)(OH)O—, —(CH₂)ₙOP(O)(SH)O—, —(CH₂CH₂O)ₙP(O)(OH)O—, and —(CH₂CH₂O)ₙP(O)(SH)O—, wherein each n is independently about 1-100, 1-50, 1-40, 1-30, 1-20, 1-10, 1-5, 2-100, 2-50, 2-40, 2-30, 2-20, 2-10, 2-5, 3-100, 3-50, 3-40, 3-30, 3-20, 3-10, 3-5, 1, 2, 3, 4, 5, 6, 7, 8, 9, 10, 11, 12, 13, 14, 15, 16, 17, 18, 19, or 20. In some embodiments, n is about 1-20. In some embodiments, n is about 3-10. In some embodiments, n is 1. In some embodiments, n is 2. In some embodiments, n is 3. In some embodiments, n is 4. In some embodiments, n is 5. In some embodiments, n is 6. In some embodiments, n is 6. In some embodiments, n is 7. In some embodiments, n is 8. In some embodiments, n is 9. In some embodiments, n is 10. In some embodiments, a linker comprises one or more units each independently selected from

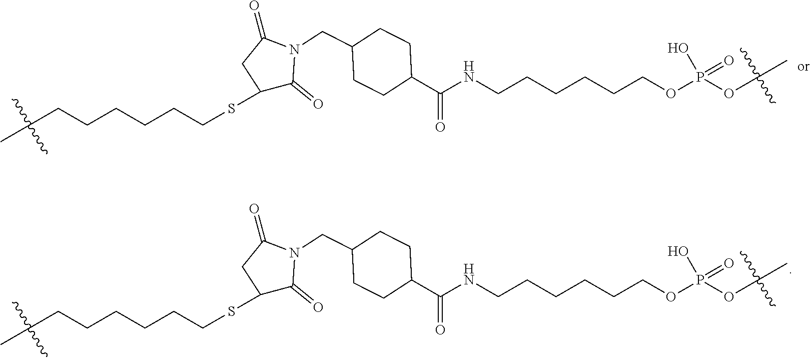 [SMILES: *CCCCCCSC1CC(=O)N(CC2CCC(C(=O)NCCCCCCOP(=O)(O)O*)CC2)C1=O ; *CCCCCCSC1CC(=O)N(CC2CCC(C(=O)NCCCCCCOP(=O)(O)O*)CC2)C1=O]

In some embodiments, for one unit, L^{k}, or a phosphate or phosphorothioate moiety is bonded to 5′-position of a 5′-end nucleoside of the first domain. In some embodiments, for one unit, L^{k}, or a phosphate or phosphorothioate moiety is bonded to 3′-position of a 3′-end nucleoside of the first domain. In some embodiments, for one unit, L^{k}, or a phosphate or phosphorothioate moiety is bonded to 5′-position of a 5′-end nucleoside of the second domain. In some embodiments, for one unit, L^{k}, or a phosphate or phosphorothioate moiety is bonded to 3′-position of a 3′-end nucleoside of the second domain. In some embodiments, for one unit, —CH₂— is bonded to 5′-position of a 5′-end nucleoside of a first domain through L^{k}. In some embodiments, —CH₂— is bonded to 3′-position of a 3′-end nucleoside of the first domain through L^{k}. In some embodiments, for one unit, —CH₂— is bonded to 5′-position of a 5′-end nucleoside of a second domain through L^{k}. In some embodiments, for one unit, —CH₂— is bonded to 3′-position of a 3′-end nucleoside of the second domain through L^{k}. In some embodiments, L^{k} has the structure of a PO linkage. In some embodiments, L^{k} has the structure of natural phosphate linkage. In some embodiments, L^{k} has the structure of a PS linkage. In some embodiments, L^{k} has the structure of a phosphorothioate internucleotidic linkage. In some embodiments, L^{k} has the structure of a PN linkage. In some embodiments, L^{k} has the structure of a phosphoryl guanidine internucleotidic linkage. In some embodiments, L^{k} has the structure of n001.

In some embodiments, linkers, and functional groups and reactive groups (e.g., for linking entities such as first and second domains), are selected based on a variety of factors, e.g., size, solubility (e.g., in aqueous solution), biocompatibility, reactivity, reaction conditions for linking, reagents/catalysts, etc. for linking, etc.

A variety of linkers are suitable for use in accordance with the present disclosure. In some embodiments, a linker is or comprises polyvinylether, polyethylene, polypropylene, polyethylene glycol (PEG), polypropylene glycol (PEG), polyvinyl alcohol (PVA), polyglycolide (PGA), polylactide (PLA), polycaprolactone (PCL), and copolymers thereof. In some embodiments, useful functional/reactive groups is an amine, sulfhydryl, carboxyl, hydroxyl, alkene (e.g., a terminal alkene), or azide group. Various multifunctional (e.g., bifunctional) linkers are also generally known in the art and can be utilized in accordance with the present disclosure, and may be either heterofunctional or homofunctional, and may include any suitable functional group, e.g., isothiocyanate, isocyanate, acyl azide, an NHS ester, sulfonyl chloride, tosyl ester, tresyl ester, aldehyde, amine, epoxide, carbonate (e.g., bis(p- nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc.

In some embodiments, an linker is or comprises an alkyl, alkenyl, alkynyl, substituted alkyl, substituted alkenyl, substituted alkynyl, repeated ethylene glycol groups, ether, thioether, urea, carbonate, amine, amide, maleimide-thioether, disulfide, phosphodiester, sulfonamide linkage, a product of a click reaction (e.g., a triazole from the azide-alkyne cycloaddition), and/or carbamate. In some embodiments, a linker may is a cleavable linker, e.g., redox cleavable linker (such as a reductively cleavable linker; e.g., a disulfide group), an acid cleavable linker (e.g., a hydrazone group, an ester group, an acetal group, and/or a ketal group), an esterase cleavable linker (e.g., an ester group), a phosphatase cleavable linker (e.g., a phosphate group), or a peptidase cleavable linker (e.g., a peptide bond), a bio-cleavable linker, e.g., comprising DNA, RNA, disulfide, amide, functionalized monosaccharides or oligosaccharides of galactosamine.

In some embodiments, a linker comprises one or more acetamido linkages, for example, bromoacetamido thiol-reactive chemistry instead of maleimide (for example, Bromoacetamido-PEG2-NHS ester). In some embodiments, a linker may exploit other chemical properties, for example, as will be readily appreciated by the skilled artisan as applied to linking moieties (e.g., bioconjugation), such as Diels-Alder; Reverse electron-demand Diels-Alder; Copper-catalyzed (CuAAC) Huisgen azide-alkyne 1,3-dipolar cycloaddition; Copper-free strained alkyne-azide (SPAAC); Staudinger ligation; Active esters including NHS, OPfp, OTfp esters; Native chemical ligation; Chemoenzymatic ligation; etc.

In some embodiments, a linker comprises an optionally substituted 1H-1,2,3-triazole ring. In some embodiments, a linker comprises an optionally substituted 1H-1,2,3-triazole ring formed from an azide and an alkyne.

In some embodiments, one or more units are independently a salt form.

In some embodiments, linkers are useful for linking entities other than first and second domains, e.g., additional chemical moieties and oligonucleotide chains.

In some embodiments, a target nucleic acid is RNA. In some embodiments, a target nucleic acid is a transcript. In some embodiments, a target nucleic acid is mRNA. In some embodiments, a target nucleic acid comprises a target adenosine which is a G to A mutation. In some embodiments, when a target adenosine is edited to inosine, the product nucleic acid provides one or more improved and/or new desired properties and/or activities, and/or reduces or removes one or more undesired properties and/or activities. For example, in some embodiments, a target adenosine is associated with a condition, disorder or disease, and after its conversion to inosine, a product nucleic acid and/or products thereof are less, or not, associated with a condition, disorder or disease.

### Base Sequences

As appreciated by those skilled in the art, structural features of the present disclosure, such as nucleobase modification, sugar modifications, internucleotidic linkage modifications, linkage phosphorus stereochemistry, etc., and combinations thereof may be utilized with various suitable base sequences to provide oligonucleotides and compositions with desired properties and/or activities. For example, oligonucleotides for adenosine modification (e.g., conversion to I in the presence of ADAR proteins) typically have sequences that are sufficiently complementary to sequences of target nucleic acids that comprise target adenosines. Nucleosides opposite to target adenosines can be present at various positions of oligonucleotides. In some embodiments, one or more opposite nucleosides are in first domains. In some embodiments, one or more opposite nucleosides are in second domains. In some embodiments, one or more opposite nucleosides are in first subdomains. In some embodiments, one or more opposite nucleosides are in second subdomains. In some embodiments, one or more opposite nucleosides are in third subdomains. Oligonucleotide of the present disclosure may target one or more target adenosines. In some embodiments, one or more opposite nucleosides are each independently in a portion which has the structure features of a second subdomain, and each independently have one or more or all structural features of opposite nucleosides as described herein. In many embodiments, e.g., for targeting G to A mutations, oligonucleotides may selectively target one and only one target adenosine for modification, e.g., by ADAR to convert into I. In some embodiments, an opposite nucleoside is closer to the 3′-end than to the 5′-end of an oligonucleotide.

In some embodiments, an oligonucleotide has abase sequence described herein (e.g., in Tables) or a portion thereof (e.g., a span of 10-50, 10-40, 10-30, 10-20, or 10, 11, 12, 13, 14, 15, 16, 17, 18, 19, 20, or at least 10, at least 15, at least 20, at least 25 contiguous nucleobases) with 0-5 (e.g., 0, 1, 2, 3, 4 or 5) mismatches, wherein each T can be independently substituted with U and vice versa. In some embodiments, an oligonucleotide comprises a base sequence described herein, or a portion thereof, wherein a portion is a span of at least 10 contiguous nucleobases, or a span of at least 15 contiguous nucleobases with 0-5 mismatches. In some embodiments, provided oligonucleotides have a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa.

In some embodiments, base sequences of oligonucleotides comprise or consist of 10-60 (e.g., about or at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55. 60; in some embodiments, at least 15; in some embodiments, at least 16; in some embodiments, at least 17; in some embodiments, at least 18; in some embodiments, at least 19; in some embodiments, at least 20; in some embodiments, at least 21; in some embodiments, at least 22; in some embodiments, at least 23; in some embodiments, at least 24; in some embodiments, at least 25; in some embodiments, at least 26; in some embodiments, at least 27; in some embodiments, at least 28; in some embodiments, at least 29; in some embodiments, at least 30; in some embodiments, at least 31; in some embodiments, at least 32; in some embodiments, at least 33; in some embodiments, at least 34; in some embodiments, at least 35) bases, optionally contiguous, of a base sequence that is identical or complementary to a base sequence of nucleic acid, e.g., a gene or a transcript (e.g., mRNA) thereof In some embodiments, the base sequence of an oligonucleotide is or comprises a sequence that is complementary to a target sequence in a gene or a transcript thereof In some embodiments, the sequence is 10, 11, 12, 13, 14, 15, 16, 17, 18, 19, 20, 21, 22, 23, 24, 25, 26, 27, 28, 29, 30, 31, 32, 33. 34, 35, 36, 37, 38, 39, 40, 45, 50, 55, 60 or more nucleobases in length.

In some embodiments, a target sequence is or comprises a characteristic sequence of a nucleic acid sequence (e.g., of an gene or a transcript thereof) in that it defines the nucleic acid sequence over others in a relevant organism; for example, a characteristic sequence is not in or has at least various mismatches from other genomic nucleic acid sequences (e.g., genes) or transcripts thereof in a relevant organism. In some embodiments, a characteristic sequence of a transcript defines that transcript over other transcripts in a relevant organism; for example, in some embodiments, a characteristic sequence is not in transcripts that are transcribed from a different nucleic acid sequence (e.g., a different gene). In some embodiments, transcript variants from a nucleic acid sequence (e.g., mRNA variants of a gene) may share a common characteristic sequence that defines them from, e.g., transcripts of other genes. In some embodiments, a characteristic sequence comprises a target adenosine. In some embodiments, an oligonucleotide selectively forms a duplex with a nucleic acid comprising a target adenosine, wherein the target adenosine is within the duplex region and can be modified by a protein such as ADAR1 or ADAR2.

Base sequences of provided oligonucleotides, as appreciated by those skilled in the art, typically have sufficient lengths and complementarity to their target nucleic acids, e.g., RNA transcripts (e.g., pre-mRNA, mature mRNA, etc.) for, e.g., site-directed editing of target adenosines. In some embodiments, an oligonucleotide is complementary to a portion of a target RNA sequence comprising a target adenosine (as appreciated by those skilled in the art. in many instances target nucleic acids are longer than oligonucleotides of the present disclosure, and complementarity may be properly assessed based on the shorter of the two, oligonucleotides). In some embodiments, the base sequence of an oligonucleotide has 90% or more identity with the base sequence of an oligonucleotide disclosed in a Table, wherein each T can be independently substituted with U and vice versa. In some embodiments, the base sequence of an oligonucleotide has 95% or more identity with the base sequence of an oligonucleotide disclosed in a Table, wherein each T can be independently substituted with U and vice versa. In some embodiments, the base sequence of an oligonucleotide comprises a continuous span of 15, 16, 17, 18, 19, 20, 21, 22, 23, 24, 25, 26, 27, 28, 29, 30, 31, 32, 33, 34, 35, 36, 37, 38, 39, 40 or more bases of an oligonucleotide disclosed in a Table, wherein each T can be independently substituted with U and vice versa, except that one or more bases within the span are abasic (e.g., a nucleobase is absent from a nucleotide).

In some embodiments, the present disclosure pertains to an oligonucleotide having a base sequence which comprises the base sequence of any oligonucleotide disclosed herein, wherein each T may be independently replaced with U and vice versa.

In some embodiments, the present disclosure pertains to an oligonucleotide having a base sequence which is the base sequence of any oligonucleotide disclosed herein, wherein each T may be independently replaced with U and vice versa.

In some embodiments, the present disclosure pertains to an oligonucleotide having a base sequence which comprises at least 15 contiguous bases of the base sequence of any oligonucleotide disclosed herein, wherein each T may be independently replaced with U and vice versa.

In some embodiments, the present disclosure pertains to an oligonucleotide having a base sequence which is at least 90% identical to the base sequence of any oligonucleotide disclosed herein, wherein each T may be independently replaced with U and vice versa.

In some embodiments, the present disclosure pertains to an oligonucleotide having a base sequence which is at least 95% identical to the base sequence of any oligonucleotide disclosed herein, wherein each T may be independently replaced with U and vice versa.

In some embodiments, a base sequence of an oligonucleotide is, comprises, or comprises 10-40, e.g., 15, 16, 17, 18, 19, 20, 21, 22, 23, 24, 25, 26, 27, 28, 29, 30, 31, 32, 33, 34, 35, 36, 37, 38, 39, 40 contiguous bases of the base sequence of any oligonucleotide describer herein, wherein each T may be independently replaced with U and vice versa.

In some embodiments, an oligonucleotide is an oligonucleotide presented in a Table herein.

In some embodiments, the base sequence of an oligonucleotide is complementary to that of a target nucleic acid, e.g., a portion comprising a target adenosine.

In some embodiments, base sequence of an oligonucleotide comprises a domain whose base sequence shares about 70%-100% (e.g., about 50%, 75%, 80%, 85%, 90%, 95% or 100%) identity with a first domain of an oligonucleotide described in a Table, wherein each T may be independently replaced with U and vice versa. In some embodiments, base sequence of an oligonucleotide comprises a domain whose sequence shares about 70%-100% (e.g., about 50%, 75%, 80%, 85%, 90%, 95% or 100%) identity with a second domain of an oligonucleotide described in a Table, wherein each T may be independently replaced with U and vice versa. In some embodiments, base sequence of an oligonucleotide shares about 70%-100% (e.g., about 50%, 75%, 80%, 85%, 90%, 95% or 100%) identity with an oligonucleotide described in a Table, wherein each T may be independently replaced with U and vice versa. In some embodiments, base sequence of an oligonucleotide comprises about or at least about 10-20 (e.g., about or at least about 10, 11, 12, 13, 14, 15, 16, 17, 18, 19, 20) consecutive nucleobases of a first domain of an oligonucleotide in a Table, wherein each T may be independently replaced with U and vice versa. In some embodiments, base sequence of an oligonucleotide comprises about or at least about 10-20 (e.g., about or at least about 10, 11, 12, 13, 14, 15, 16, 17, 18, 19, 20) consecutive nucleobases of a second domain of an oligonucleotide in a Table, wherein each T may be independently replaced with U and vice versa. In some embodiments, base sequence of an oligonucleotide comprises about or at least about 10-20 (e.g., about or at least about 10, 11, 12, 13, 14, 15, 16, 17, 18, 19, 20) consecutive nucleobases of an oligonucleotide in a Table, wherein each T may be independently replaced with U and vice versa. In some embodiments, base sequence of an oligonucleotide comprises base sequence of a first domain of an oligonucleotide in a Table, wherein each T may be independently replaced with U and vice versa. In some embodiments, base sequence of an oligonucleotide comprises base sequence of a second domain of an oligonucleotide in a Table, wherein each T may be independently replaced with U and vice versa. In some embodiments, base sequence of an oligonucleotide is or comprises base sequence of an oligonucleotide in a Table, wherein each T may be independently replaced with U and vice versa.

In some embodiments, an oligonucleotide comprises a base sequence or portion thereof (e.g., a portion comprising 10-40, e.g., 15, 16, 17, 18, 19, 20, 21, 22, 23, 24, 25, 26, 27, 28, 29, 30, 31, 32, 33, 34, 35, 36, 37, 38, 39, 40 nucleobases) described in any of the Tables, wherein each T may be independently replaced with U and vice versa, and/or a sugar, nucleobase, and/or internucleotidic linkage modification and/or stereochemistry, and/or a pattern thereof described in any of the Tables, and/or an additional chemical moiety (in addition to an oligonucleotide chain, e.g., a target moiety, a lipid moiety, a carbohydrate moiety, etc.) described in any of the Tables.

In some embodiments, the terms “complementary,” “fully complementary” and “substantially complementary” may be used with respect to the base matching between an oligonucleotide and a target sequence, as will be understood by those skilled in the art from the context of their uses. It is noted that substitution of T for U, or vice versa, generally does not alter the amount of complementarity. As used herein, an oligonucleotide that is “substantially complementary” to a target sequence is largely or mostly complementary but not necessarily 100% complementary. In some embodiments, a sequence (e.g., an oligonucleotide ) which is substantially complementary has one or more, e.g., 1, 2, 3, 4 or 5 mismatches when maximally aligned to its target sequence. In some embodiments, an oligonucleotide has a base sequence which is substantially complementary to a target sequence of a target nucleic acid. In some embodiments, an oligonucleotide has a base sequence which is substantially complementary to the complement of the sequence of an oligonucleotide disclosed herein. As appreciated by those skilled in the art, in some embodiments, sequences of oligonucleotides need not be 100% complementary to their targets for oligonucleotides to perform their functions (e.g., converting A to I in a nucleic acid. In some embodiments, a mismatch is well tolerated at the 5′ and/or 3′ end or the middle of an oligonucleotide. In some embodiments, one or more mismatches are preferred for adenosine modification. In some embodiments, oligonucleotides comprise portions for complementarity to target nucleic acids, and optionally portions that are not primarily for complementarity to target nucleic acids; for example, in some embodiments, oligonucleotides may comprise portions for protein binding. In some embodiments, base sequences of provided oligonucleotides are fully complementary to their target sequences (A-T/U and C-G base pairing). In some embodiments, base sequences of provided oligonucleotides are fully complementary to their target sequences (A-T/U and C-G base pairing) except at a nucleoside opposite to a target nucleoside (e.g., adenosine).

In some embodiments, the present disclosure provides an oligonucleotide comprising a sequence found in an oligonucleotide described in a Table, wherein one or more U is independently and optionally replaced with T or vice versa. In some embodiments, an oligonucleotide can comprise at least one T and/or at least one U. In some embodiments, the present disclosure provides an oligonucleotide comprising a sequence found in an oligonucleotide described in a Table herein, wherein the said sequence has over 50% identity with the sequence of the oligonucleotide described in a Table. In some embodiments, the present disclosure provides an oligonucleotide whose base sequence is the sequence of an oligonucleotide disclosed in a Table, wherein each T may be independently replaced with U and vice versa. In some embodiments, the present disclosure provides an oligonucleotide comprising a sequence found in an oligonucleotide in a Table, wherein the oligonucleotides have a pattern of backbone linkages, pattern of backbone chiral centers, and/or pattern of backbone phosphorus modifications of the same oligonucleotide or another oligonucleotide in a Table herein.

In some embodiments, the disclosure provides an oligonucleotide having a base sequence which is, comprises, or comprises a portion of the base sequence of an oligonucleotide disclosed herein, e.g., in a Table, wherein each T may be independently replaced with U and vice versa, wherein the oligonucleotide optionally further comprises a chemical modification, stereochemistry, format, an additional chemical moiety described herein (e.g., a targeting moiety, lipid moiety, carbohydrate moiety, etc.), and/or another structural feature.

In some embodiments, a “portion” (e.g., of a base sequence or a pattern of modifications or other structural element) is at least 5, 6, 7, 8, 9, 10, 11, 12, 13, 14, 15, 16, 17, 18, 19, or 20 monomeric units long.

Those skilled in the art reading the present disclosure will appreciate that technologies herein may be utilized to target various target nucleic acids comprising target adenosine for editing. In some embodiments, a target nucleic acid is a transcript of a PiZZ allele. In some embodiments, a target adenosine is . . . atcgacAagaaagggactgaagc . . . . In some embodiments, oligonucleotides of the present disclosure have suitable base sequences so that they have sufficient complementarity to selectively form duplexes with a portion of a transcript that comprise the target adenosine for editing.

As described herein, nucleosides opposite to target nucleosides (e.g., A) can be positioned at various locations. In some embodiments, an opposite nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 5′-end of an oligonucleotide. In some embodiments, it is at position 3 or more from the 5′-end of an oligonucleotide. In some embodiments, it is at position 4 or more from the 5′-end of an oligonucleotide. In some embodiments, it is at position 5 or more from the 5′-end of an oligonucleotide. In some embodiments, it is at position 6 or more from the 5′-end of an oligonucleotide. In some embodiments, it is at position 7 or more from the 5′-end of an oligonucleotide. In some embodiments, it is at position 8 or more from the 5′-end of an oligonucleotide. In some embodiments, it is at position 9 or more from the 5′-end of an oligonucleotide. In some embodiments, it is at position 10 or more from the 5′-end of an oligonucleotide. In some embodiments, an opposite nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 3′-end of an oligonucleotide. In some embodiments, it is at position 3 or more from the 3′-end of an oligonucleotide. In some embodiments, it is at position 4 or more from the 3′-end of an oligonucleotide. In some embodiments, it is at position 5 or more from the 3′-end of an oligonucleotide. In some embodiments, it is at position 6 or more from the 3′-end of an oligonucleotide. In some embodiments, it is at position 7 or more from the 3′-end of an oligonucleotide. In some embodiments, it is at position 8 or more from the 3′-end of an oligonucleotide. In some embodiments, it is at position 9 or more from the 3′-end of an oligonucleotide. In some embodiments, it is at position 10 or more from the 3′-end of an oligonucleotide. In some embodiments, nucleobases at position 1 from the 5′-end and/or the 3′-end are complementary to corresponding nucleobases in target sequences when aligned for maximum complementarity. In some embodiments, certain positions, e.g., position 6, 7, or 8, may provide higher editing efficiency.

As examples, certain oligonucleotides comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties, etc., are presented in Table 1, below. Among other things, these oligonucleotides may be utilized to correct a G to A mutation in a gene or gene product (e.g., by converting A to I). In some embodiments, listed in Tables are stereorandom oligonucleotide compositions. In some embodiments, the present disclosure provides chirally controlled oligonucleotide compositions.

In some embodiments, a base sequence is or comprises a particular sequence. In some embodiments, a base sequence is complementary to a base sequence that is or comprises a base sequence that is complementary to a particular sequence. In some embodiments, a base sequence is or comprise a sequence that differs from a particular sequence at no more than 1, 2, 3, 4, or 5 positions. In some embodiments, a base sequence is or comprise a sequence that differs from about 15-30 (e.g., 15-25, 15-20, 20-30, 15, 16, 17, 18, 19, 20, 21, 22, 23, 24, 25, 26, 27, 28, 29 or 30) consecutive nucleobases of a particular sequence at no more than 1, 2, 3, 4, or 5 positions. In some embodiments, a base sequence is or comprise a sequence that differs from a particular sequence at no more than 1 position. In some embodiments, a base sequence is or comprise a sequence that differs from a particular sequence at no more than 2 positions. In some embodiments, a base sequence is or comprise a sequence that differs from a particular sequence at no more than 3 positions. In some embodiments, a base sequence is or comprise a sequence that differs from a particular sequence at no more than 4 positions. In some embodiments, a base sequence is or comprise a sequence that differs from a particular sequence at no more than 5 positions. In some embodiments, a particular sequence is or comprises a base sequence selected from Table 1. In some embodiments, a particular sequence is or comprises 5-30, 10-30, 15-30, 20-30, or 25-30 (e.g., 5, 6, 7, 8, 9, 10, 11, 12, 13, 14, 15, 16, 17, 18, 19, 20, 21, 22, 23, 24, 25, 26, 27, 28, 29 or 30) consecutive bases in a base sequence selected from Table 1. In some embodiments, a particular sequence is or comprises 10 consecutive bases in a base sequence selected from Table 1. In some embodiments, a particular sequence is or comprises 11 consecutive bases in a base sequence selected from Table 1. In some embodiments, a particular sequence is or comprises 12 consecutive bases in a base sequence selected from Table 1. In some embodiments, a particular sequence is or comprises 13 consecutive bases in a base sequence selected from Table 1. In some embodiments, a particular sequence is or comprises 14 consecutive bases in a base sequence selected from Table 1. In some embodiments, a particular sequence is or comprises 15 consecutive bases in a base sequence selected from Table 1. In some embodiments, a particular sequence is or comprises 16 consecutive bases in a base sequence selected from Table 1. In some embodiments, a particular sequence is or comprises 17 consecutive bases in abase sequence selected from Table 1. In some embodiments, a particular sequence is or comprises 18 consecutive bases in a base sequence selected from Table 1. In some embodiments, a particular sequence is or comprises 19 consecutive bases in a base sequence selected from Table 1. In some embodiments, a particular sequence is or comprises 20 consecutive bases in a base sequence selected from Table 1. In some embodiments, a particular sequence is or comprises 21 consecutive bases in a base sequence selected from Table 1. In some embodiments, a particular sequence is or comprises 22 consecutive bases in a base sequence selected from Table 1. In some embodiments, a particular sequence is or comprises 23 consecutive bases in a base sequence selected from Table 1. In some embodiments, a particular sequence is or comprises 24 consecutive bases in abase sequence selected from Table 1. In some embodiments, a particular sequence is or comprises 25 consecutive bases in a base sequence selected from Table 1. In some embodiments, a particular sequence is or comprises 26 consecutive bases in a base sequence selected from Table 1. In some embodiments, a particular sequence is or comprises 27 consecutive bases in a base sequence selected from Table 1. In some embodiments, a particular sequence is or comprises 28 consecutive bases in a base sequence selected from Table 1. In some embodiments, a particular sequence is or comprises 29 consecutive bases in a base sequence selected from Table 1. In some embodiments, a particular sequence is or comprises 30 consecutive bases in a base sequence selected from Table 1.

### Gap

As described herein, in some embodiments, base sequence of a first domain of an oligonucleotide is complementary to base sequence of a first portion of a target nucleic acid, and base sequence of a second domain of an oligonucleotide is complementary to base sequence of a second portion of a target nucleic acid. In some embodiments, a first domain of an oligonucleotide can hybridize to a first portion of a target nucleic acid, and a second domain of an oligonucleotide can hybridize to a second portion of a target nucleic acid. In some embodiments, a first portion and a second portion is separated by a gap in base sequence.

In some embodiments, length of the gap is about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 nucleobases. In some embodiments, length of a gap is about 2-100,000, 2-50,000, 2-10,000, 2-5,000, 3-100,000, 3-50.000, 3-10,000, 3-5,000, 4-100,000, 4-50,000, 4-10,000, 4-5,000, 5-100,000, 5-50,000, 5-10,000, 5-5,000, 10-100,000, 10-50,000, 10-10,000, 10-5,000, 20-100,000, 20-50,000, 20-10,000, 20-5,000, 50-100,000, 50-50,000, 50-10,000, 50-5,000, 100-100,000, 100-50,000, 100-10,000, or 100-5,000 nucleobases. Among other things, the present disclosure surprisingly demonstrate that oligonucleotides comprising first and second domains independently with complimentary base sequences to first and second portions separated by large gaps, e.g., in some embodiments, about 10 nucleobases or more; in some embodiments, about 20 nucleobases or more; in some embodiments, about 50 nucleobases or more; in some embodiments, about 100 nucleobases or more, can provide various activities including target adenosine editing.

In some embodiments, length of a gap is 1 nucleobase or more. In some embodiments, length of a gap is about or at least about 2 nucleobases. In some embodiments, length of a gap is about or at least about 3 nucleobases. In some embodiments, length of a gap is about or at least about 4 nucleobases. In some embodiments, length of a gap is about or at least about 5 nucleobases. In some embodiments, length of a gap is about or at least about 6 nucleobases. In some embodiments, length of a gap is about or at least about 7 nucleobases. In some embodiments, length of a gap is about or at least about 8 nucleobases. In some embodiments, length of a gap is about or at least about 9 nucleobases. In some embodiments, length of a gap is about or at least about 10 nucleobases. In some embodiments, length of a gap is about or at least about 11 nucleobases. In some embodiments, length of a gap is about or at least about 12 nucleobases. In some embodiments, length of a gap is about or at least about 13 nucleobases. In some embodiments, length of a gap is about or at least about 14 nucleobases. In some embodiments, length of a gap is about or at least about 15 nucleobases. In some embodiments, length of a gap is about or at least about 16 nucleobases. In some embodiments, length of a gap is about or at least about 17 nucleobases. In some embodiments, length of a gap is about or at least about 18 nucleobases. In some embodiments, length of a gap is about or at least about 19 nucleobases. In some embodiments, length of a gap is about or at least about 20 nucleobases. In some embodiments, length of a gap is about or at least about 25 nucleobases. In some embodiments, length of a gap is about or at least about 30 nucleobases. In some embodiments, length of a gap is about or at least about 40 nucleobases. In some embodiments, length of a gap is about or at least about 50 nucleobases. In some embodiments, length of a gap is about or at least about 60 nucleobases. In some embodiments, length of a gap is about or at least about 70 nucleobases. In some embodiments, length of a gap is about or at least about 80 nucleobases. In some embodiments, length of a gap is about or at least about 90 nucleobases. In some embodiments, length of a gap is about or at least about 100 nucleobases. In some embodiments, length of a gap is about or at least about 110 nucleobases. In some embodiments, length of a gap is about or at least about 120 nucleobases. In some embodiments, length of a gap is about or at least about 130 nucleobases. In some embodiments, length of a gap is about or at least about 140 nucleobases. In some embodiments, length of a gap is about or at least about 150 nucleobases. In some embodiments, length of a gap is about or at least about 200 nucleobases. In some embodiments, length of a gap is about or at least about 300 nucleobases. In some embodiments, length of a gap is about or at least about 400 nucleobases. In some embodiments, length of a gap is about or at least about 500 nucleobases. In some embodiments, length of a gap is about or at least about 600 nucleobases. In some embodiments, length of a gap is about or at least about 700 nucleobases. In some embodiments, length of a gap is about or at least about 800 nucleobases. In some embodiments, length of a gap is about or at least about 900 nucleobases. In some embodiments, length of a gap is about or at least about 1000 nucleobases. In some embodiments, length of a gap is about or at least about 1500 nucleobases. In some embodiments, length of a gap is about or at least about 2000 nucleobases. In some embodiments, length of a gap is about or at least about 3000 nucleobases. In some embodiments, length of a gap is about or at least about 4000 nucleobases. In some embodiments, length of a gap is about or at least about 5000 nucleobases. In some embodiments, length of a gap is about or at least about 10000 nucleobases.

In some embodiments, each nucleobase in the gap is independently optionally substituted A, T, C, G, or U, or an optionally substituted tautomer of A, T, C, G or U. In some embodiments, each nucleobase in the gap is independently optionally substituted A, T, C, G, or U. In some embodiments, each nucleobase in the gap is independently A, T, C, G. or U.

TABLE 1
Example oligonucleotides and/or compositions.

| ID | Description | Base Sequence |
|---|---|---|
| ADR- | mC[n001R]mG[Ssp]mA[Ssp][fl2r]C[Ssp][fl2r]U[Ssp][fl2r]U[Ssp][fl2r]A[Ssp][fl2r]C[Ssp][fl2r]U[Ssp][fl2r] | CGACUUACUCC |
| 0104859 | C[Ssp][fl2r]C[Ssp][fl2r]A[Ssp][fl2r]A[Ssp][fl2r]G[Ssp][fl2r]A[Ssp][fl2r]U[Ssp][fl2r]C[Ssp][fl2r]U[Ssp][f12 | AAGAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | [fl2r]A[n001R][fl2r]C[Ssp][fl2r]U[Ssp][fl2r]C[Ssp][fl2r]C[Ssp][fl2r]C[Ssp][fl2r]A[Ssp][fl2r]G[Ssp][f]2r]G[ | ACUCCCAGGGA |
| 0105002 | Ssp][fl2r]G[Ssp][fl2r]A[Ssp][fl2r]G[Ssp][fl2r]A[Ssp][fl2r]C[Ssp][fl2r]C[Ssp]mA[Ssp]mA[Ssp]mA[Ssp]mG | GACCAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]G[n001R][fl2r]U[Ssp][fl2r]G[Ssp][fl2r]U[Ssp][fl2r]G[Ssp][fl2r]C[Ssp][fl2r]A[Ssp][fl2r]C[Ssp][fl2r]U[ | GUGUGCACUUU |
| 0105003 | Ssp][fl2r]U[Ssp][fl2r]U[Ssp][fl2r]U[Ssp][fl2r]A[Ssp][fl2r]U[Ssp][fl2r]U[Ssp]mC[Ssp]mA[Ssp]mA[Ssp]mG | UAUUCAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]U[n001R][fl2r]U[Ssp][fl2r]U[Ssp][fl2r]A[Ssp][fl2r]A[Ssp][fl2r]G[Ssp][fl2r]G[Ssp][fl2r]U[Ssp][fl2r]G[ | UUUAAGGUGUG |
| 0105004 | Ssp][fl2r]U[Ssp][fl2r]G[Ssp][fl2r]C[Ssp][fl2r]A[Ssp][fl2r]C[Ssp][fl2r]U[Ssp]mU[Ssp]mA[Ssp]mA[Ssp]mG | CACUUAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]U[n001R][fl2r]G[Ssp][fl2r]C[Ssp][fl2r]A[Ssp][fl2r]U[Ssp][fl2r]U[Ssp][fl2r]A[Ssp][fl2r]C[Ssp][fl2r]A[ | UGCAUUACAUA |
| 0105007 | Ssp][fl2r]U[Ssp][fl2r]A[Ssp][fl2r]A[Ssp][fl2r]U[Ssp][fl2r]U[Ssp][fl2r]U[Ssp]mA[Ssp]mA[Ssp]mA[Ssp]mG | AUUUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]A[n001R][fl2r]A[Ssp][fl2r]U[Ssp][fl2r]U[Ssp][fl2r]U[Ssp][fl2r]U[Ssp][fl2r]G[Ssp][fl2r]C[Ssp][fl2r]A[ | AAUUUUGCAUU |
| 0105008 | Ssp][fl2r]U[Ssp][fl2r]U[Ssp][fl2r]A[Ssp][fl2r]C[Ssp][fl2r]A[Ssp][fl2r]U[Ssp]mA[Ssp]mA[Ssp]mA[Ssp]mG | ACAUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]A[n001R][fl2r]U[Ssp][fl2r]U[Ssp][fl2r]A[Ssp][fl2r]A[Ssp][fl2r]G[Ssp][fl2r]G[Ssp][fl2r]C[Ssp][fl2r]G[ | AUUAAGGCGAA |
| 0105009 | Ssp][fl2r]A[Ssp][fl2r]A[Ssp][fl2r]G[Ssp][fl2r]A[Ssp][fl2r]U[Ssp][fl2r]U[Ssp]mA[Ssp]mA[Ssp]mA[Ssp]mG | GAUUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]U[n001R][fl2r]C[Ssp][fl2r]A[Ssp][fl2r]U[Ssp][fl2r]U[Ssp][fl2r]C[Ssp][fl2r]A[Ssp][fl2r]A[Ssp][fl2r]A[ | UCAUUCAAAAU |
| 0105010 | Ssp][fl2r]A[Ssp][fl2r]U[Ssp][fl2r]A[Ssp][fl2r]A[Ssp][fl2r]A[Ssp][fl2r]A[Ssp]mC[Ssp]mA[Ssp]mA[Ssp]mG | AAAACAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]A[n001R][fl2r]C[Ssp][fl2r]U[Ssp][fl2r]C[Ssp][fl2r]C[Ssp][fl2r]C[Ssp][fl2r]A[Ssp][fl2r]G[Ssp][fl2r]G[ | ACUCCCAGGGA |
| 0105011 | Ssp][fl2r]G[Ssp][fl2r]A[Ssp][fl2r]G[Ssp][fl2r]A[Ssp][fl2r]C[Ssp][fl2r]C[Ssp]mA[Ssp]mA[Ssp]mA[Ssp]mG | GACCAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]G[n001R][fl2r]U[Ssp][fl2r]G[Ssp][fl2r]U[Ssp][fl2r]G[Ssp][fl2r]C[Ssp][fl2r]A[Ssp][fl2r]C[Ssp][fl2r]U[ | GUGUGCACUUU |
| 0105012 | Ssp][fl2r]U[Ssp][fl2r]U[Ssp][fl2r]U[Ssp][fl2r]A[Ssp][fl2r]U[Ssp][fl2r]U[Ssp]mC[Ssp]mA[Ssp]mA[Ssp]mG | UAUUCAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]U[n001R][fl2r]U[Ssp][fl2r]U[Ssp][fl2r]A[Ssp][fl2r]A[Ssp][fl2r]G[Ssp][fl2r]G[Ssp][fl2r]U[Ssp][fl2r]G[ | UUUAAGGUGUG |
| 0105013 | Ssp][fl2r]U[Ssp][fl2r]G[Ssp][fl2r]C[Ssp][fl2r]A[Ssp][fl2r]C[Ssp][fl2r]U[Ssp]mU[Ssp]mA[Ssp]mA[Ssp]mG | CACUUAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACCU |
| | mC[Ssp]mC[Ssp]mU[n001R]mC | C |
| ADR- | [fl2r]U[n001R][fl2r]U[Ssp][fl2r]A[Ssp][fl2r]C[Ssp][fl2r]A[Ssp][fl2r]U[Ssp][fl2r]A[Ssp][fl2r]A[Ssp][fl2r]U[ | UUACAUAAUUU |
| 0105014 | Ssp][fl2r]U[Ssp][fl2r]U[Ssp][fl2r]A[Ssp][fl2r]C[Ssp][fl2r]A[Ssp][fl2r]C[Ssp]mG[Ssp]mU[Ssp]mA[Ssp]mA | ACACGUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]C[n001R][fl2r]A[Ssp][fl2r]U[Ssp][fl2r]U[Ssp][fl2r]A[Ssp][fl2r]C[Ssp][fl2r]A[Ssp][fl2r]U[Ssp][fl2r]A[ | CAUUACAUAAU |
| 0105015 | Ssp][fl2r]A[Ssp][fl2r]U[Ssp][fl2r]U[Ssp][fl2r]U[Ssp][fl2r]A[Ssp][fl2r]C[Ssp]mA[Ssp]mU[Ssp]mA[Ssp]mA | UUACAUAAGCA |
| | [Ssp][Ssp][Ssp]A[Ssp]A[Ssp]mU[Ssp]mG[Ssp][fl2r][Ssp]dC[Ssp]dA[n001R]mU[Ssp]mC[Ssp] | AUGCCAUCAC |
| | mA[n001R]mC | |
| ADR- | [fl2r]U[n001R][fl2r]G[Ssp][fl2r]C[Ssp][fl2r]A[Ssp][fl2r]U[Ssp][fl2r]U[Ssp][fl2r]A[Ssp][fl2r]C[Ssp][fl2r]A[ | UGCAUUACAUA |
| 0105016 | Ssp][fl2r]U[Ssp][fl2r]A[Ssp][fl2r]A[Ssp][fl2r]U[Ssp][fl2r]U[Ssp][fl2r]U[Ssp]mA[Ssp]mU[Ssp]mA[Ssp]mA | AUUUAUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]A[n001R][fl2r]A[Ssp][fl2r]U[Ssp][fl2r]U[Ssp][fl2r]U[Ssp][fl2r]U[Ssp][fl2r]G[Ssp][fl2r]C[Ssp][fl2r]A[ | AAUUUUGCAUU |
| 0105017 | Ssp][fl2r]U[Ssp][fl2r]U[Ssp][fl2r]A[Ssp][fl2r]C[Ssp][fl2r]A[Ssp][fl2r]U[Ssp]mA[Ssp]mU[Ssp]mA[Ssp]mA | ACAUAUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]A[n001R][fl2r]U[Ssp][fl2r]U[Ssp][fl2r]A[Ssp][fl2r]A[Ssp][fl2r]G[Ssp][fl2r]G[Ssp][fl2r]C[Ssp][fl2r]G[ | AUUAAGGCGAA |
| 0105018 | Ssp][fl2r]A[Ssp][fl2r]A[Ssp][fl2r]G[Ssp][fl2r]A[Ssp][fl2r]U[Ssp][fl2r]U[Ssp]mA[Ssp]mU[Ssp]mA[Ssp]mA | GAUUAUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]A[n001R][fl2r]C[Ssp][fl2r]U[Ssp][fl2r]C[Ssp][fl2r]C[Ssp][fl2r]C[Ssp][fl2r]A[Ssp][fl2r]G[Ssp][fl2r]G[ | ACUCCCAGGGA |
| 0105020 | Ssp][fl2r]G[Ssp][fl2r]A[Ssp][fl2r]G[Ssp][fl2r]A[Ssp][fl2r]C[Ssp][fl2r]C[Ssp]mA[Ssp]mU[Ssp]mA[Ssp]mA | GACCAUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]G[n001R][fl2r]U[Ssp][fl2r]G[Ssp][fl2r]U[Ssp][fl2r]G[Ssp][fl2r]C[Ssp][fl2r]A[Ssp][fl2r]C[Ssp][fl2r]U[ | GUGUGCACUUU |
| 0105021 | Ssp][fl2r]U[Ssp][fl2r]U[Ssp][fl2r]U[Ssp][fl2r]A[Ssp][fl2r]U[Ssp][fl2r]U[Ssp]mC[Ssp]mU[Ssp]mA[Ssp]mA | UAUUCUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]U[n001R][fl2r]U[Ssp][fl2r]U[Ssp][fl2r]A[Ssp][fl2r]A[Ssp][fl2r]G[Ssp][fl2r]G[Ssp][fl2r]U[Ssp][fl2r]G[ | UUUAAGGUGUG |
| 0105022 | Ssp][fl2r]U[Ssp][fl2r]G[Ssp][fl2r]C[Ssp][fl2r]A[Ssp][fl2r]C[Ssp][fl2r]U[Ssp]mU[Ssp]mU[Ssp]mA[Ssp]mA | CACUUUAAGCA |
| | [Ssp]mG[Ssp]mC[Ssp]mA[Ssp]mA[Ssp]mU[Ssp]mG[Ssp][fl2r]C[Ssp]dC[Ssp]dA[n001R]mU[Ssp]mC[Ssp] | AUGCCAUCAC |
| | mA[n001R]mC | |
| ADR- | [fl2r]U[n001R][fl2r]U[Ssp][fl2r]A[Ssp][fl2r]C[Ssp][fl2r]A[Ssp][fl2r]U[Ssp][fl2r]A[Ssp][fl2r]A[Ssp][fl2r]U[ | UUACAUAAUUU |
| 0105023 | Ssp][fl2r]U[Ssp][fl2r]U[Ssp][fl2r]A[Ssp][fl2r]C[Ssp][fl2r]A[Ssp][fl2r]C[Ssp]mG[Ssp]mU[Ssp]mU[Ssp]mA | ACACGUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]C[n001R][f]2r]A[Ssp][fl2r]U[Ssp][fl2r]U[Ssp][fl2r]A[Ssp][fl2r]C[Ssp][fl2r]A[Ssp][fl2r]U[Ssp][fl2r]A[ | CAUUACAUAAU |
| 0105024 | Ssp][fl2r]A[Ssp][fl2r]U[Ssp][fl2r]U[Ssp][fl2r]U[Ssp][fl2r]A[Ssp][fl2r]C[Ssp]mA[Ssp]mU[Ssp]mU[Ssp]mA | UUACAUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]U[n001R][fl2r]G[Ssp][fl2r]C[Ssp][fl2r]A[Ssp][fl2r]U[Ssp][fl2r]U[Ssp][fl2r]A[Ssp][fl2r]C[Ssp][fl2r]A[ | UGCAUUACAUA |
| 0105025 | Ssp][fl2r]U[Ssp][fl2r]A[Ssp][fl2r]A[Ssp][fl2r]U[Ssp][fl2r]U[Ssp][fl2r]U[Ssp]mA[Ssp]mU[Ssp]mU[Ssp]mA | AUUUAUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]A[n001R][fl2r]A[Ssp][fl2r]U[Ssp][fl2r]U[Ssp][fl2r]U[Ssp][fl2r]U[Ssp][fl2r]G[Ssp][fl2r]C[Ssp][fl2r]A[ | AAUUUUGCAUU |
| 0105026 | Ssp][fl2r]U[Ssp][fl2r]U[Ssp][fl2r]A[Ssp][fl2r]C[Ssp][fl2r]A[Ssp][fl2r]U[Ssp]mA[Ssp]mU[Ssp]mU[Ssp]mA | ACAUAUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]U[n001R][fl2r]C[Ssp][fl2r]A[Ssp][fl2r]U[Ssp][fl2r]U[Ssp][fl2r]C[Ssp][fl2r]A[Ssp][fl2r]A[Ssp][fl2r]A[ | UCAUUCAAAAU |
| 0105028 | Ssp][fl2r]A[Ssp][fl2r]U[Ssp][fl2r]A[Ssp][fl2r]A[Ssp][fl2r]A[Ssp][fl2r]A[Ssp]mC[Ssp]mU[Ssp]mU[Ssp]mA | AAAACUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]A[n001R][fl2r]C[Ssp][fl2r]U[Ssp][fl2r]C[Ssp][fl2r]C[Ssp][fl2r]C[Ssp][fl2r]A[Ssp][fl2r]G[Ssp][fl2r]G[ | ACUCCCAGGGA |
| 0105029 | Ssp][fl2r]G[Ssp][fl2r]A[Ssp][fl2r]G[Ssp][fl2r]A[Ssp][fl2r]C[Ssp][fl2r]C[Ssp]mA[Ssp]mU[Ssp]mU[Ssp]mA | GACCAUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]G[n001R][fl2r]U[Ssp][fl2r]G[Ssp][fl2r]U[Ssp][fl2r]G[Ssp][fl2r]C[Ssp][fl2r]A[Ssp][fl2r]C[Ssp][fl2r]U[ | GUGUGCACUUU |
| 0105030 | Ssp][fl2r]U[Ssp][fl2r]U[Ssp][fl2r]U[Ssp][fl2r]A[Ssp][fl2r]U[Ssp][fl2r]U[Ssp]mC[Ssp]mU[Ssp]mU[Ssp]mA | UAUUCUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r][n001R][fl2r]U[Ssp][fl2r]U[Ssp][fl2r]A[Ssp][fl2r]A[Ssp][fl2r]G[Ssp][fl2r]G[Ssp][fl2r]U[Ssp][fl2r]G[ | UUUAAGGUGUG |
| 0105031 | Ssp][fl2r]U[Ssp][fl2r]G[Ssp][fl2r]C[Ssp][fl2r]A[Ssp][fl2r]C[Ssp][fl2r]U[Ssp]mU[Ssp]mU[Ssp]mU[Ssp]mA | CACUUUUAAGC |
| | [Ssp]mA[Ssp]mG[Ssp]mC[Ssp]mA[Ssp]mA[Ssp]mU[Ssp]mG[Ssp][fl2r]C[Ssp]dC[Ssp]dA[n001R]mU[Ssp] | AAUGCCAUCAC |
| | mC[Ssp]mA[n001R]mC | |
| ADR- | [fl2r]C[n001R][fl2r]A[Ssp][fl2r]U[Ssp][fl2r]U[Ssp][fl2r]A[Ssp][fl2r]C[Ssp][fl2r]A[Ssp][fl2r]U[Ssp][fl2r]A[ | CAUUACAUAAU |
| 0105032 | Ssp][fl2r]A[Ssp][fl2r]U[Ssp][fl2r]U[Ssp][fl2r]U[Ssp][fl2r]A[Ssp][fl2r]C[Ssp]mA[Ssp]mA[Ssp]mA[Ssp]mG | UUACAAAGCAA |
| | [Ssp]mC[Ssp]mA[Ssp]mA[Ssp]mU[Ssp]mG[Ssp][fl2r][Ssp]dC[Ssp]dA[n001R]mU[Ssp]mC[Ssp]mA[n001 | UGCCAUCAC |
| | R]mC | |
| ADR- | [fl2r]U[n001R][fl2r]G[Ssp][fl2r]C[Ssp][fl2r]A[Ssp][fl2r]U[Ssp][fl2r]U[Ssp][fl2r]A[Ssp][fl2r]C[Ssp][fl2r]A[ | UGCAUUACAUA |
| 0105033 | Ssp][fl2r]U[Ssp][fl2r]A[Ssp][fl2r]A[Ssp][fl2r]U[Ssp][fl2r]U[Ssp][fl2r]U[Ssp]mA[Ssp]mA[Ssp]mA[Ssp]mG | AUUUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]A[n001R][fl2r]A[Ssp][fl2r]U[Ssp][fl2r]U[Ssp][fl2r]U[Ssp][fl2r]U[Ssp][fl2r]G[Ssp][fl2r]C[Ssp][fl2r]A[ | AAUUUUGCAUU |
| 0105034 | Ssp][fl2r]U[Ssp][fl2r]U[Ssp][fl2r]A[Ssp][fl2r]C[Ssp][fl2r]A[Ssp][fl2r]U[Ssp]mA[Ssp]mA[Ssp]mA[Ssp]mG | ACAUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]A[n001R][fl2r]U[Ssp][fl2r]U[Ssp][fl2r]A[Ssp][fl2r]A[Ssp][fl2r]G[Ssp][fl2r]G[Ssp][fl2r]C[Ssp][fl2r]G[ | AUUAAGGCGAA |
| 0105035 | Ssp][fl2r]A[Ssp][fl2r]A[Ssp][fl2r]G[Ssp][fl2r]A[Ssp][fl2r]U[Ssp][fl2r]U[Ssp]mA[Ssp]mA[Ssp]mA[Ssp]mG | GAUUAAAGCAA |
| | [Ssp][Ssp]A[Ssp]A[Ssp]mU[Ssp]mG[Ssp][fl2r][Ssp]dC[Ssp]dA[n001R]mU[Ssp]mC[Ssp]mA[n001 | UGCCAUCAC |
| | R]mC | |
| ADR- | [fl2r]U[n001R][fl2r]C[Ssp][fl2r]A[Ssp][fl2r]U[Ssp][fl2r]U[Ssp][fl2r]C[Ssp][fl2r]A[Ssp][fl2r]A[Ssp][fl2r]A[ | UCAUUCAAAAU |
| 0105036 | Ssp][fl2r]A[Ssp][fl2r]U[Ssp][fl2r]A[Ssp][fl2r]A[Ssp][fl2r]A[Ssp][fl2r]A[Ssp]mC[Ssp]mA[Ssp]mA[Ssp]mG | AAAACAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]A[n001R][fl2r]C[Ssp][fl2r]U[Ssp][fl2r]C[Ssp][fl2r]C[Ssp][fl2r]C[Ssp][fl2r]A[Ssp][fl2r]G[Ssp][fl2r]G[ | ACUCCCAGGGA |
| 0105037 | Ssp][fl2r]G[Ssp][fl2r]A[Ssp][fl2r]G[Ssp][fl2r]A[Ssp][fl2r]C[Ssp][fl2r]C[Ssp]mA[Ssp]mA[Ssp]mA[Ssp]mG | GACCAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]G[n001R][fl2r]U[Ssp][fl2r]G[Ssp][fl2r]U[Ssp][fl2r]G[Ssp][fl2r]C[Ssp][fl2r]A[Ssp][fl2r]C[Ssp][fl2r]U[ | GUGUGCACUUU |
| 0105038 | Ssp][fl2r]U[Ssp][fl2r]U[Ssp][fl2r]U[Ssp][fl2r]A[Ssp][fl2r]U[Ssp][fl2r]U[Ssp]mC[Ssp]mA[Ssp]mA[Ssp]mG | UAUUCAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]U[n001R][fl2r]U[Ssp][fl2r]U[Ssp][fl2r]A[Ssp][fl2r]A[Ssp][fl2r]G[Ssp][fl2r]G[Ssp][fl2r]U[Ssp][fl2r]G[ | UUUAAGGUGUG |
| 0105039 | Ssp][fl2r]U[Ssp][fl2r]G[Ssp][fl2r]C[Ssp][fl2r]A[Ssp][fl2r]C[Ssp][fl2r]U[Ssp]mU[Ssp]mA[Ssp]mA[Ssp]mG | CACUUAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | [fl2r]U[n001R][fl2r]U[Ssp][fl2r]A[Ssp][fl2r]C[Ssp][fl2r]A[Ssp][fl2r]U[Ssp][fl2r]A[Ssp][fl2r]A[Ssp][fl2r]U[ | UUACAUAAUUU |
| 0105040 | Ssp][fl2r]U[Ssp][fl2r]U[Ssp][fl2r]A[Ssp][fl2r]C[Ssp][fl2r]A[Ssp][fl2r]C[Ssp]mG[Ssp]mA[Ssp]mA[Ssp]mG | ACACGAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]C[n001R][fl2r]A[Ssp][fl2r]U[Ssp][fl2r]U[Ssp][fl2r]A[Ssp][fl2r]C[Ssp][fl2r]A[Ssp][fl2r]U[Ssp][fl2r]A[ | CAUUACAUAAU |
| 0105041 | Ssp][fl2r]A[Ssp][fl2r]U[Ssp][fl2r]U[Ssp][fl2r]U[Ssp][fl2r]A[Ssp][fl2r]C[Ssp]mA[Ssp]mA[Ssp]mA[Ssp]mG | UUACAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]U[n001R][fl2r]G[Ssp][fl2r]C[Ssp][fl2r]A[Ssp][fl2r]U[Ssp][fl2r]U[Ssp][fl2r]A[Ssp][fl2r]C[Ssp][fl2r]A[ | UGCAUUACAUA |
| 0105042 | Ssp][fl2r]U[Ssp][fl2r]A[Ssp][fl2r]A[Ssp][fl2r]U[Ssp][fl2r]U[Ssp][fl2r]U[Ssp]mA[Ssp]mA[Ssp]mA[Ssp]mG | AUUUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]A[n001R][fl2r]A[Ssp][fl2r]U[Ssp][fl2r]U[Ssp][fl2r]U[Ssp][fl2r]U[Ssp][fl2r]G[Ssp][fl2r]C[Ssp][fl2r]A[ | AAUUUUGCAUU |
| 0105043 | Ssp][fl2r]U[Ssp][fl2r]U[Ssp][fl2r]A[Ssp][fl2r]C[Ssp][fl2r]A[Ssp][fl2r]U[Ssp]mA[Ssp]mA[Ssp]mA[Ssp]mG | ACAUAAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r]A[n001R][fl2r]U[Ssp][fl2r]U[Ssp][fl2r]A[Ssp][fl2r]A[Ssp][fl2r]G[Ssp][fl2r]G[Ssp][fl2r]C[Ssp][fl2r]G[ | AUUAAGGCGAA |
| 0105044 | Ssp][fl2r]A[Ssp][fl2r]A[Ssp][fl2r]G[Ssp][fl2r]A[Ssp][fl2r]U[Ssp][fl2r]U[Ssp]mA[Ssp]mA[Ssp]mA[Ssp]mG | GAUUAAAGCAA |
| | [Ssp][Ssp]A[Ssp]A[Ssp]mU[Ssp]mG[Ssp][fl2r][Ssp]dC[Ssp]dA[n001R]mU[Ssp]mC[Ssp]mA[Ssp] | UGCCAUCACC |
| | mC[n001R]mC | |
| ADR- | [fl2r]U[n001R][fl2r]C[Ssp][fl2r]A[Ssp][fl2r]U[Ssp][fl2r]U[Ssp][fl2r]C[Ssp][fl2r]A[Ssp][fl2r]A[Ssp][fl2r]A[ | UCAUUCAAAAU |
| 0105045 | Ssp][fl2r]A[Ssp][fl2r]U[Ssp][fl2r]A[Ssp][fl2r]A[Ssp][fl2r]A[Ssp][fl2r]A[Ssp]mC[Ssp]mA[Ssp]mA[Ssp]mG | AAAACAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[Ssp] | UGCCAUCACC |
| | mC[n001R]mC | |
| ADR- | [fl2r][[n001R][fl2r]U[Ssp][fl2r]A[Ssp][fl2r]C[Ssp][fl2r]A[Ssp][fl2r]U[Ssp][fl2r]A[Ssp][fl2r]A[Ssp][fl2r]U[ | UUACAUAAUUU |
| 0105046 | Ssp][fl2r]U[Ssp][fl2r]U[Ssp][fl2r]A[Ssp][fl2r]C[Ssp][fl2r]A[Ssp][fl2r]C[Ssp]mG[Ssp]mA[Ssp]mA[Ssp]mG | ACACGAAGCAA |
| | [Ssp]mC[Ssp]mA[Ssp]mA[Ssp]mU[Ssp]mG[Ssp][fl2r]C[Ssp]dC[Ssp]dA[n001R]mU[Ssp]mC[Ssp]mA[n001 | UGCCAUCAC |
| | R]mC | |
| ADR- | mC[n001R]mU[Ssp]mG[Ssp][fl2r]C[Ssp][fl2r]C[Ssp][fl2r]U[Ssp][fl2r]C[Ssp][fl2r]A[Ssp][fl2r]A[Ssp][fl2r] | CUGCCUCAAUG |
| 0105461 | U[Ssp][fl2r]G[Ssp][fl2r]C[Ssp][fl2r]C[Ssp][f]2r]U[Ssp][fl2r]A[Ssp][fl2r]C[Ssp][fl2r]C[Ssp][fl2r]G[Ssp][fl2r | CCUACCGCACA |
| | ]C[Ssp][fl2r]A[Ssp][fl2r]C[Ssp][fl2r]A[Ssp][fl2r]G[Ssp]dC[Ssp]d[3nU][Ssp]dC[n001R]mGp[fl2r]G[Ssp]m | GC[3nU]CGGGC |
| | G[n001R]mC | |
| ADR- | mA[n001R]mU[Ssp]mG[Ssp][fl2r]A[Ssp][fl2r]C[Ssp][fl2r]C[Ssp][fl2r]C[Ssp][fl2r]U[Ssp][fl2r]A[Ssp][fl2r] | AUGACCCUACU |
| 0105462 | C[Ssp][fl2r]U[Ssp][fl2r]G[Ssp][fl2r]A[Ssp][fl2r]A[Ssp][fl2r]G[Ssp][fl2r]C[Ssp][fl2r]C[Ssp][fl2r]G[Ssp][fl2 | GAAGCCGCACA |
| | r]C[Ssp][fl2r]A[Ssp][fl2r]C[Ssp][fl2r]A[Ssp][fl2r]G[Ssp]dC[Ssp]d[3nU][Ssp]dC[n001R]mGp[fl2r]G[Ssp]m | GC[3nU]CGGGC |
| | G[n001R]mC | |
| ADR- | mC[n001R]mA[Ssp]mA[Ssp][fl2r]U[Ssp][fl2r]U[Ssp][fl2r]U[Ssp][fl2r]U[Ssp][fl2r]C[Ssp][fl2r]U[Ssp][fl2r] | CAAUUUUCUUU |
| 0105463 | U[Ssp][fl2r]U[Ssp][fl2r]U[Ssp][fl2r]A[Ssp][fl2r]A[Ssp][fl2r]A[Ssp][fl2r]U[Ssp][fl2r]A[Ssp][fl2r]C[Ssp][f12 | UAAAUACAUUU |
| | r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2r]U[Ssp]m | UT[3nU]ACUCC |
| | C[n001R]mC | |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0105464 | C[Ssp][fl2r]U[Ssp][fl2r]C[Ssp][fl2r]U[Ssp][fl2r]C[Ssp][fl2r]A[Ssp][fl2r]U[Ssp][fl2r]A[Ssp][fl2r]C[Ssp][fl2r | CUCAUACAUUU |
| | ]A[Ssp][fl2r]U[Ssp][fl2r]U[Ssp][fl2r]U[Ssp][fl2r]U[Ssp]dT[Ssp]d[3nU][Ssp]dA[n001R]mCp[fl2r]U[Ssp]m | UT[3nU]ACUCC |
| | C[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0105465 | C[Ssp][fl2r]C[Ssp][fl2r]U[Ssp][fl2r]U[Ssp][f]2r]U[Ssp][fl2r]C[Ssp][fl2r]U[Ssp][fl2r]A[Ssp][fl2r]C[Ssp][fl2r | UUUCUACAUUU |
| | ]A[Ssp][fl2r]U[Ssp][fl2r]U[Ssp][fl2r]U[Ssp][fl2r]U[Ssp]dT[Ssp]d[3nU][Ssp]dA[n001R]mCp[fl2r]U[Ssp]m | UT[3nU]ACUCC |
| | C[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0105466 | A[Ssp][fl2r]U[Ssp][fl2r]U[Ssp][fl2r]U[Ssp][fl2r]U[Ssp][fl2r]C[Ssp][fl2r]U[Ssp][fl2r]A[Ssp][fl2r]C[Ssp][fl2 | UUUCUACAUUU |
| | r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2r]U[Ssp]m | UT[3nU]ACUCC |
| | C[n001R]mC | |
| ADR- | mU[n001R]mA[Ssp]mU[Ssp][fl2r]C[Ssp][fl2r]U[Ssp][fl2r]U[Ssp][fl2r]C[Ssp][fl2r]C[Ssp][fl2r]A[Ssp][fl2r] | UAUCUUCCAAU |
| 0105467 | A[Ssp][fl2r]U[Ssp][fl2r]U[Ssp][fl2r]U[Ssp][fl2r]U[Ssp][fl2r]C[Ssp][fl2r]U[Ssp][fl2r]A[Ssp][fl2r]C[Ssp][fl2 | UUUCUACAUUU |
| | r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2r]U[Ssp]m | UT[3nU]ACUCC |
| | C[n001R]mC | |
| ADR- | mU[n001R]mA[Ssp]mU[Ssp][fl2r]C[Ssp][fl2r]U[Ssp][fl2r]U[Ssp][fl2r]C[Ssp][fl2r]C[Ssp][fl2r]A[Ssp][fl2r] | UAUCUUCCAAU |
| 0105488 | A[Ssp][fl2r]U[Ssp][fl2r]U[Ssp][fl2r]U[Ssp][fl2r]U[Ssp][fl2r]C[Ssp][fl2r]U[Ssp][fl2r]C[Ssp][fl2r]A[Ssp][fl2 | UUUCUCAAACU |
| | r]A[Ssp][fl2r]A[Ssp][fl2r]C[Ssp][fl2r]U[Ssp][fl2r]U[Ssp]dC[Ssp]d[3nU][Ssp]dA[n001R]mUp[fl2r]C[Ssp]m | UC[3nU]AUCUU |
| | U[n001R]mU | |
| ADR- | mU[n001R]mC[Ssp]mU[Ssp][fl2r]U[Ssp][fl2r]C[Ssp][fl2r]C[Ssp][fl2r]A[Ssp][fl2r]G[Ssp][fl2r]U[Ssp][fl2r] | UCUUCCAGUUU |
| 0105489 | U[Ssp][fl2r]U[Ssp][fl2r]U[Ssp][fl2r]C[Ssp][fl2r]C[Ssp][fl2r]A[Ssp][fl2r]C[Ssp][fl2r]U[Ssp][fl2r]U[Ssp][f12 | UCCACUUCAAA |
| | r]C[Ssp][fl2r]A[Ssp][fl2r]A[Ssp][fl2r]A[Ssp][fl2r]C[Ssp]dT[Ssp]d[3nU][Ssp]dC[n001R]mUp[fl2r]A[Ssp]m | CT[3nU]CUAUC |
| | U[n001R]mC | |
| ADR- | mU[n001R]mA[Ssp]mU[Ssp][fl2r]C[Ssp][fl2r]U[Ssp][fl2r]U[Ssp][fl2r]C[Ssp][fl2r]C[Ssp][fl2r]A[Ssp][fl2r] | UAUCUUCCAAU |
| 0105490 | A[Ssp][fl2r]U[Ssp][fl2r]U[Ssp][fl2r]U[Ssp][fl2r]U[Ssp][fl2r]C[Ssp][fl2r]C[Ssp][fl2r]U[Ssp][f]2r]U[Ssp][f12 | UUUCCUUCAAA |
| | r]C[Ssp][fl2r]A[Ssp][fl2r]A[Ssp][fl2r]A[Ssp][fl2r]C[Ssp]dT[Ssp]d[3nU][Ssp]dC[n001R]mUp[fl2r]A[Ssp]m | CT[3nU]CUAUC |
| | U[n001R]mC | |
| ADR- | mG[n001R]mC[Ssp]mA[Ssp][fl2r]C[Ssp][fl2r]U[Ssp][fl2r]U[Ssp][fl2r]A[Ssp][fl2r]U[Ssp][fl2r]C[Ssp][fl2r] | GCACUUAUCCC |
| 0105525 | C[Ssp][fl2r]C[Ssp][fl2r]U[Ssp][fl2r]A[Ssp][fl2r]A[Ssp][fl2r]C[Ssp][fl2r]A[Ssp][fl2r]U[Ssp][fl2r]G[Ssp][f12 | UAACAUGCAAU |
| | r]C[Ssp][fl2r]A[Ssp][fl2r]A[Ssp][fl2r]U[Ssp][fl2r]A[Ssp]dC[Ssp]d[3nU][Ssp]dG[n001R]mCp[fl2r]A[Ssp]m | AC[3nU]GCAGA |
| | G[n001R]mA | |
| ADR- | mA[n001R]mG[Ssp]mG[Ssp][fl2r]A[Ssp][fl2r]G[Ssp][fl2r]A[Ssp][fl2r]A[Ssp][fl2r]A[Ssp][fl2r]G[Ssp][fl2r] | AGGAGAAAGUG |
| 0105526 | U[Ssp][fl2r]G[Ssp][fl2r]C[Ssp][fl2r]C[Ssp][fl2r]A[Ssp][fl2r]U[Ssp][fl2r]A[Ssp][fl2r]U[Ssp][fl2r]G[Ssp][f12 | CCAUAUGCAAU |
| | r]C[Ssp][fl2r]A[Ssp][fl2r]A[Ssp][fl2r]U[Ssp][fl2r]A[Ssp]dC[Ssp]d[3nU][Ssp]dG[n001R]mCp[fl2r]A[Ssp]m | AC[3nU]GCAGA |
| | G[n001R]mA | |
| ADR- | mU[n001R]mU[Ssp]mG[Ssp][fl2r]G[Ssp][fl2r]G[Ssp][fl2r]C[Ssp][fl2r]U[Ssp][fl2r]U[Ssp][fl2r]C[Ssp][fl2r] | UUGGGCUUCUU |
| 0105552 | U[Ssp][fl2r]U[Ssp][fl2r]A[Ssp][fl2r]G[Ssp][fl2r]G[Ssp][fl2r]U[Ssp][fl2r]U[Ssp][fl2r]U[Ssp][fl2r]U[Ssp][fl2 | AGGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mA[n001R]mU[Ssp]mU[Ssp][fl2r]U[Ssp][fl2r]G[Ssp][fl2r]G[Ssp][fl2r]G[Ssp][fl2r]C[Ssp][fl2r]U[Ssp][fl2r] | AUUUGGGCUUC |
| 0105553 | U[Ssp][fl2r]C[Ssp][fl2r]U[Ssp][fl2r]U[Ssp][fl2r]A[Ssp][fl2r]G[Ssp][fl2r]U[Ssp][fl2r]U[Ssp][fl2r]U[Ssp][f12 | UUAGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mA[n001R]mG[Ssp]mA[Ssp][fl2r]U[Ssp][fl2r]U[Ssp][fl2r]U[Ssp][fl2r]G[Ssp][fl2r]G[Ssp][fl2r]G[Ssp][fl2r] | AGAUUUGGGCU |
| 0105554 | C[Ssp][fl2r]U[Ssp][fl2r]U[Ssp][fl2r]C[Ssp][fl2r]U[Ssp][fl2r]U[Ssp][fl2r]U[Ssp][fl2r]U[Ssp][fl2r]U[Ssp][fl2 | UCUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0105555 | G[Ssp][fl2r]G[Ssp][fl2r]C[Ssp][fl2r]U[Ssp][fl2r]U[Ssp][fl2r]C[Ssp][fl2r]U[Ssp][fl2r]U[Ssp][fl2r]U[Ssp][f12 | CUUCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mG[Ssp][fl2r]G[Ssp][fl2r]A[Ssp][fl2r]G[Ssp][fl2r]A[Ssp][fl2r]U[Ssp][fl2r]U[Ssp][fl2r] | UGGGAGAUUUG |
| 0105556 | U[Ssp][fl2r]G[Ssp][fl2r]G[Ssp][fl2r]G[Ssp][fl2r]C[Ssp][fl2r]U[Ssp][fl2r]U[Ssp][fl2r]U[Ssp][fl2r]U[Ssp][fl2 | GGCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mU[Ssp]mU[Ssp][fl2r]G[Ssp][fl2r]G[Ssp][fl2r]G[Ssp][fl2r]A[Ssp][fl2r]G[Ssp][fl2r]A[Ssp][fl2r] | UUUGGGAGAUU |
| 0105557 | U[Ssp][fl2r]U[Ssp][fl2r]U[Ssp][fl2r]G[Ssp][fl2r]G[Ssp][fl2r]G[Ssp][fl2r]U[Ssp][fl2r]U[Ssp][fl2r]U[Ssp][fl2 | UGGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mC[Ssp]mU[Ssp][fl2r]U[Ssp][fl2r]U[Ssp][fl2r]G[Ssp][fl2r]G[Ssp][fl2r]G[Ssp][fl2r]A[Ssp][fl2r] | GCUUUGGGAGA |
| 0105558 | G[Ssp][fl2r]A[Ssp][fl2r]U[Ssp][fl2r]U[Ssp][f]2r]U[Ssp][fl2r]G[Ssp][fl2r]U[Ssp][fl2r]U[Ssp][fl2r]U[Ssp][f12 | UUUGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0105559 | G[Ssp][fl2r]A[Ssp][fl2r]G[Ssp][fl2r]A[Ssp][fl2r]U[Ssp][fl2r]U[Ssp][fl2r]U[Ssp][fl2r]U[Ssp][fl2r]U[Ssp][f12 | GAUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mG[Ssp][fl2r]A[Ssp][fl2r]G[Ssp][fl2r]C[Ssp][fl2r]U[Ssp][fl2r]U[Ssp][fl2r]U[Ssp][fl2r] | UGGAGCUUUGG |
| 0105560 | G[Ssp][fl2r]G[Ssp][fl2r]G[Ssp][fl2r]A[Ssp][fl2r]G[Ssp][fl2r]A[Ssp][fl2r]U[Ssp][fl2r]U[Ssp][fl2r]U[Ssp][fl2 | GAGAUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mC[n001R]mC[Ssp]mU[Ssp][fl2r]G[Ssp][fl2r]G[Ssp][fl2r]A[Ssp][fl2r]G[Ssp][fl2r]C[Ssp][fl2r]U[Ssp][fl2r] | CCUGGAGCUUU |
| 0105561 | U[Ssp][fl2r]U[Ssp][fl2r]G[Ssp][fl2r]G[Ssp][fl2r]G[Ssp][fl2r]A[Ssp][fl2r]U[Ssp][fl2r]U[Ssp][fl2r]U[Ssp][fl2 | GGGAUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mU[Ssp]mC[Ssp][fl2r]C[Ssp][fl2r]U[Ssp][fl2r]G[Ssp][fl2r]G[Ssp][fl2r]A[Ssp][fl2r]G[Ssp][fl2r] | UUCCUGGAGCU |
| 0105562 | C[Ssp][fl2r]U[Ssp][fl2r]U[Ssp][fl2r]U[Ssp][fl2r]G[Ssp][fl2r]G[Ssp][fl2r]U[Ssp][fl2r]U[Ssp][f]2r]U[Ssp][f12 | UUGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mA[n001R]mG[Ssp]mU[Ssp][fl2r]U[Ssp][fl2r]C[Ssp][fl2r]C[Ssp][fl2r]U[Ssp][fl2r]G[Ssp][fl2r]G[Ssp][fl2r] | AGUUCCUGGAG |
| 0105563 | A[Ssp][fl2r]G[Ssp][fl2r]C[Ssp][fl2r]U[Ssp][fl2r]U[Ssp][fl2r]U[Ssp][fl2r]U[Ssp][fl2r]U[Ssp][fl2r]U[Ssp][fl2 | CUUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mC[n001R]mC[Ssp]mA[Ssp][fl2r]G[Ssp][fl2r]U[Ssp][fl2r]U[Ssp][fl2r]C[Ssp][fl2r]C[Ssp][fl2r]U[Ssp][fl2r] | CCAGUUCCUGG |
| 0105564 | G[Ssp][fl2r]G[Ssp][fl2r]A[Ssp][fl2r]G[Ssp][fl2r]C[Ssp][fl2r]U[Ssp][fl2r]U[Ssp][fl2r]U[Ssp][fl2r]U[Ssp][fl2 | AGCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mC[Ssp][fl2r]C[Ssp][fl2r]A[Ssp][fl2r]G[Ssp][fl2r]U[Ssp][fl2r]U[Ssp][fl2r]C[Ssp][fl2r] | UGCCAGUUCCU |
| 0105565 | C[Ssp][fl2r]U[Ssp][fl2r]G[Ssp][fl2r]G[Ssp][fl2r]A[Ssp][fl2r]G[Ssp][fl2r]U[Ssp][fl2r]U[Ssp][fl2r]U[Ssp][fl2 | GGAGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mC[Ssp]mU[Ssp][fl2r]G[Ssp][fl2r]C[Ssp][fl2r]C[Ssp][fl2r]A[Ssp][fl2r]G[Ssp][fl2r]U[Ssp][fl2r] | UCUGCCAGUUC |
| 0105566 | U[Ssp][fl2r]C[Ssp][fl2r]C[Ssp][fl2r]U[Ssp][fl2r]G[Ssp][fl2r]G[Ssp][fl2r]U[Ssp][fl2r]U[Ssp][fl2r]U[Ssp][f12 | CUGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mC[n001R]mC[Ssp]mU[Ssp][fl2r]C[Ssp][fl2r]U[Ssp][fl2r]G[Ssp][fl2r]C[Ssp][fl2r]C[Ssp][fl2r]A[Ssp][fl2r] | CCUCUGCCAGU |
| 0105567 | G[Ssp][fl2r]U[Ssp][fl2r]U[Ssp][f]2r]C[Ssp][fl2r]C[Ssp][fl2r]U[Ssp][fl2r]U[Ssp][fl2r]U[Ssp][fl2r]U[Ssp][fl2 | UCCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mG[Ssp]mC[Ssp][fl2r]C[Ssp][fl2r]U[Ssp][fl2r]C[Ssp][fl2r]U[Ssp][fl2r]G[Ssp][fl2r]C[Ssp][fl2r] | GGCCUCUGCCA |
| 0105568 | C[Ssp][fl2r]A[Ssp][fl2r]G[Ssp][fl2r]U[Ssp][fl2r]U[Ssp][fl2r]C[Ssp][fl2r]U[Ssp][fl2r]U[Ssp][fl2r]U[Ssp][f12 | GUUCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mC[n001R]mC[Ssp]mG[Ssp][fl2r]G[Ssp][fl2r]C[Ssp][fl2r]C[Ssp][fl2r]U[Ssp][fl2r]C[Ssp][fl2r]U[Ssp][fl2r] | CCGGCCUCUGC |
| 0105569 | G[Ssp][fl2r]C[Ssp][fl2r]C[Ssp][fl2r]A[Ssp][fl2r]G[Ssp][fl2r]U[Ssp][fl2r]U[Ssp][fl2r]U[Ssp][fl2r]U[Ssp][f12 | CAGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0105570 | C[Ssp][fl2r]U[Ssp][fl2r]G[Ssp][fl2r]C[Ssp][fl2r]C[Ssp][fl2r]A[Ssp][fl2r]U[Ssp][fl2r]U[Ssp][fl2r]U[Ssp][f12 | GCCAUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mU[Ssp]mC[Ssp][fl2r]C[Ssp][fl2r]C[Ssp][fl2r]C[Ssp][fl2r]G[Ssp][fl2r]G[Ssp][fl2r]C[Ssp][fl2r] | GUCCCCGGCCU |
| 0105571 | C[Ssp][fl2r]U[Ssp][fl2r]C[Ssp][fl2r]U[Ssp][fl2r]G[Ssp][fl2r]C[Ssp][fl2r]U[Ssp][fl2r]U[Ssp][fl2r]U[Ssp][fl2 | CUGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mC[Ssp]mG[Ssp][fl2r]U[Ssp][fl2r]C[Ssp][fl2r]C[Ssp][fl2r]C[Ssp][fl2r]C[Ssp][fl2r]G[Ssp][fl2r] | GCGUCCCCGGC |
| 0105572 | G[Ssp][fl2r]C[Ssp][fl2r]C[Ssp][fl2r]U[Ssp][fl2r]C[Ssp][fl2r]U[Ssp][fl2r]U[Ssp][fl2r]U[Ssp][fl2r]U[Ssp][f12 | CUCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mG[Ssp]mG[Ssp][fl2r]C[Ssp][fl2r]G[Ssp][fl2r]U[Ssp][fl2r]C[Ssp][fl2r]C[Ssp][fl2r]C[Ssp][fl2r] | GGGCGUCCCCG |
| 0105573 | C[Ssp][fl2r]G[Ssp][fl2r]G[Ssp][fl2r]C[Ssp][fl2r]C[Ssp][fl2r]U[Ssp][fl2r]U[Ssp][fl2r]U[Ssp][fl2r]U[Ssp][f12 | GCCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mG[Ssp][fl2r]G[Ssp][fl2r]G[Ssp][fl2r]C[Ssp][fl2r]G[Ssp][fl2r]U[Ssp][fl2r]C[Ssp][fl2r] | UGGGGCGUCCC |
| 0105574 | C[Ssp][fl2r]C[Ssp][fl2r]C[Ssp][fl2r]G[Ssp][fl2r]G[Ssp][fl2r]C[Ssp][fl2r]U[Ssp][fl2r]U[Ssp][fl2r]U[Ssp][fl2r | CGGCUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCG |
| | n001R]mG | |
| ADR- | mU[n001R]mU[Ssp]mU[Ssp][fl2r]G[Ssp][fl2r]G[Ssp][fl2r]G[Ssp][fl2r]G[Ssp][fl2r]C[Ssp][fl2r]G[Ssp][fl2r] | UUUGGGGCGUC |
| 0105575 | U[Ssp][fl2r]C[Ssp][fl2r]C[Ssp][fl2r]C[Ssp][fl2r]C[Ssp][fl2r]G[Ssp][fl2r]U[Ssp][fl2r]U[Ssp][fl2r]U[Ssp][fl2r | CCCGUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCG |
| | [n001R]mG | |
| ADR- | mC[n001R]mC[Ssp]mU[Ssp][fl2r]U[Ssp][fl2r]U[Ssp][fl2r]G[Ssp][fl2r]G[Ssp][fl2r]G[Ssp][fl2r]G[Ssp][fl2r] | CCUUUGGGGCG |
| 0105576 | C[Ssp][fl2r]G[Ssp][fl2r]U[Ssp][f]2r]C[Ssp][f]2r]C[Ssp][fl2r]C[Ssp][fl2r]U[Ssp][fl2r]U[Ssp][fl2r]U[Ssp][fl2r | UCCCUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCG |
| | [n001R]mG | |
| ADR- | mG[n001R]mG[Ssp]mU[Ssp][fl2r]G[Ssp][fl2r]C[Ssp][fl2r]C[Ssp][fl2r]G[Ssp][fl2r]C[Ssp][fl2r]U[Ssp][fl2r] | GGUGCCGCUCC |
| 0105577 | C[Ssp][fl2r]C[Ssp][fl2r]C[Ssp][fl2r]U[Ssp][fl2r]U[Ssp][fl2r]U[Ssp][fl2r]U[Ssp][fl2r]U[Ssp][fl2r]U[Ssp][fl2 | CUUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mG[Ssp][fl2r]G[Ssp][fl2r]U[Ssp][fl2r]C[Ssp][f]2r]U[Ssp][fl2r]C[Ssp][fl2r]G[Ssp][fl2r] | UGGGUCUCGUG |
| 0105578 | U[Ssp][fl2r]G[Ssp][fl2r]G[Ssp][fl2r]U[Ssp][fl2r]G[Ssp][fl2r]C[Ssp][fl2r]U[Ssp][fl2r]U[Ssp][fl2r]U[Ssp][fl2 | GUGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mG[n001R]mU[Ssp]mG[Ssp][fl2r]G[Ssp][fl2r]C[Ssp][fl2r]C[Ssp][fl2r]G[Ssp][fl2r]C[Ssp][fl2r]C[Ssp][fl2r] | GUGGCCGCCUU |
| 0105579 | U[Ssp][fl2r]U[Ssp][fl2r]G[Ssp][fl2r]G[Ssp][fl2r]G[Ssp][fl2r]U[Ssp][fl2r]U[Ssp][fl2r]U[Ssp][fl2r]U[Ssp][fl2 | GGGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mA[n001R]mC[Ssp]mC[Ssp][fl2r]C[Ssp][fl2r]U[Ssp][fl2r]C[Ssp][fl2r]U[Ssp][fl2r]G[Ssp][fl2r]A[Ssp][fl2r] | ACCCUCUGACG |
| 0105580 | C[Ssp][fl2r]G[Ssp][fl2r]U[Ssp][fl2r]G[Ssp][fl2r]G[Ssp][fl2r]C[Ssp][fl2r]U[Ssp][fl2r]U[Ssp][fl2r]U[Ssp][fl2 | UGGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mC[Ssp]mA[Ssp][fl2r]C[Ssp][fl2r]C[Ssp][fl2r]U[Ssp][fl2r]G[Ssp][fl2r]C[Ssp][f]2r]A[Ssp][fl2r] | UCACCUGCACA |
| 0105581 | C[Ssp][fl2r]A[Ssp][fl2r]C[Ssp][fl2r]C[Ssp][fl2r]C[Ssp][fl2r]U[Ssp][fl2r]U[Ssp][fl2r]U[Ssp][fl2r]U[Ssp][fl2r | CCCUUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCG |
| | [n001R]mG | |
| ADR- | mA[n001R]mG[Ssp]mG[Ssp][fl2r]A[Ssp][fl2r]C[Ssp][fl2r]C[Ssp][fl2r]C[Ssp][fl2r]U[Ssp][fl2r]U[Ssp][fl2r] | AGGACCCUUUU |
| 0105582 | U[Ssp][fl2r]U[Ssp][fl2r]C[Ssp][fl2r]A[Ssp][fl2r]C[Ssp][fl2r]C[Ssp][fl2r]U[Ssp][fl2r]U[Ssp][fl2r]U[Ssp][fl2 | CACCUUUCUGC |
| | r]C[Ssp][fl2][Ssp][fl2][Ssp][fl2][Ssp][fl2][Ssp]dC[Ssp]d[3nU][Ssp]dC[n001R]mGp[fl2r]C[Ssp]m | UC[3nU]CGCCG |
| | C[n001R]mG | |
| ADR- | mU[n001R]mG[Ssp]mG[Ssp][fl2r]G[Ssp][fl2r]C[Ssp][fl2r]U[Ssp][fl2r]U[Ssp][fl2r]C[Ssp][fl2r]U[Ssp][fl2r] | UGGGCUUCUUA |
| 0105583 | U[Ssp][fl2r]A[Ssp][fl2r]G[Ssp][fl2r]G[Ssp][fl2r]U[Ssp][fl2r]G[Ssp][fl2r]G[Ssp][fl2r]U[Ssp][fl2r]U[Ssp][fl2 | GGUGGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mU[Ssp]mU[Ssp][fl2r]G[Ssp][fl2r]G[Ssp][fl2r]G[Ssp][fl2r]C[Ssp][fl2r]U[Ssp][fl2r]U[Ssp][fl2r] | UUUGGGCUUCU |
| 0105584 | C[Ssp][fl2r]U[Ssp][fl2r]U[Ssp][fl2r]A[Ssp][fl2r]G[Ssp][fl2r]G[Ssp][fl2r]U[Ssp][fl2r]U[Ssp][fl2r]U[Ssp][fl2 | UAGGUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mA[Ssp]mU[Ssp][fl2r]U[Ssp][fl2r]U[Ssp][fl2r]G[Ssp][fl2r]G[Ssp][fl2r]G[Ssp][fl2r]C[Ssp][fl2r] | GAUUUGGGCUU |
| 0105585 | U[Ssp][fl2r]U[Ssp][fl2r]C[Ssp][fl2r]U[Ssp][fl2r]U[Ssp][fl2r]A[Ssp][fl2r]G[Ssp][fl2r]U[Ssp][fl2r]U[Ssp][fl2 | CUUAGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mG[Ssp]mG[Ssp][fl2r]A[Ssp][fl2r]G[Ssp][fl2r]A[Ssp][fl2r]U[Ssp][fl2r]U[Ssp][fl2r]U[Ssp][fl2r] | GGGAGAUUUGG |
| 0105587 | G[Ssp][fl2r]G[Ssp][fl2r]G[Ssp][fl2r]C[Ssp][fl2r]U[Ssp][fl2r]U[Ssp][fl2r]C[Ssp][fl2r]U[Ssp][fl2r]U[Ssp][fl2 | GCUUCUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mU[Ssp]mG[Ssp][fl2r]G[Ssp][fl2r]G[Ssp][fl2r]A[Ssp][fl2r]G[Ssp][fl2r]A[Ssp][fl2r]U[Ssp][fl2r] | UUGGGAGAUUU |
| 0105588 | U[Ssp][fl2r]U[Ssp][fl2r]G[Ssp][fl2r]G[Ssp][fl2r]G[Ssp][fl2r]C[Ssp][fl2r]U[Ssp][fl2r]U[Ssp][fl2r]U[Ssp][f12 | GGGCUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mU[Ssp]mU[Ssp][fl2r]U[Ssp][fl2r]G[Ssp][fl2r]G[Ssp][fl2r]G[Ssp][fl2r]A[Ssp][fl2r]G[Ssp][fl2r] | CUUUGGGAGAU |
| 0105589 | A[Ssp][fl2r]U[Ssp][fl2r]U[Ssp][fl2r]U[Ssp][fl2r]G[Ssp][fl2r]G[Ssp][fl2r]G[Ssp][fl2r]U[Ssp][fl2r]U[Ssp][f12 | UUGGGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mA[n001R]mG[Ssp]mC[Ssp][fl2r]U[Ssp][fl2r]U[Ssp][fl2r]U[Ssp][fl2r]G[Ssp][fl2r]G[Ssp][fl2r]G[Ssp][fl2r] | AGCUUUGGGAG |
| 0105590 | A[Ssp][fl2r]G[Ssp][fl2r]A[Ssp][fl2r]U[Ssp][fl2r]U[Ssp][fl2r]U[Ssp][fl2r]G[Ssp][fl2r]U[Ssp][fl2r]U[Ssp][fl2 | AUUUGUUUCUG |
| | r]U[Ssp][fl2r]C[Ssp][f]2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mG[Ssp]mA[Ssp][fl2r]G[Ssp][fl2r]C[Ssp][fl2r]U[Ssp][fl2r]U[Ssp][fl2r]U[Ssp][fl2r]G[Ssp][fl2r] | GGAGCUUUGGG |
| 0105591 | G[Ssp][fl2r]G[Ssp][fl2r]A[Ssp][fl2r]G[Ssp][fl2r]A[Ssp][fl2r]U[Ssp][fl2r]U[Ssp][fl2r]U[Ssp][fl2r]U[Ssp][f12 | AGAUUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mU[Ssp]mG[Ssp][fl2r]G[Ssp][fl2r]A[Ssp][fl2r]G[Ssp][fl2r]C[Ssp][fl2r]U[Ssp][fl2r]U[Ssp][fl2r] | CUGGAGCUUUG |
| 0105592 | U[Ssp][fl2r]G[Ssp][fl2r]G[Ssp][fl2r]G[Ssp][fl2r]A[Ssp][fl2r]G[Ssp][fl2r]A[Ssp][fl2r]U[Ssp][fl2r]U[Ssp][fl2 | GGAGAUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mC[Ssp]mC[Ssp][fl2r]U[Ssp][fl2r]G[Ssp][fl2r]G[Ssp][fl2r]A[Ssp][fl2r]G[Ssp][fl2r]C[Ssp][fl2r] | UCCUGGAGCUU |
| 0105593 | U[Ssp][fl2r]U[Ssp][fl2r]U[Ssp][f]2r]G[Ssp][fl2r]G[Ssp][fl2r]G[Ssp][fl2r]A[Ssp][fl2r]U[Ssp][fl2r]U[Ssp][fl2 | UGGGAUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mU[Ssp]mU[Ssp][fl2r]C[Ssp][fl2r]C[Ssp][fl2r]U[Ssp][fl2r]G[Ssp][fl2r]G[Ssp][fl2r]A[Ssp][fl2r] | GUUCCUGGAGC |
| 0105594 | G[Ssp][fl2r]C[Ssp][fl2r]U[Ssp][fl2r]U[Ssp][fl2r]U[Ssp][fl2r]G[Ssp][fl2r]G[Ssp][fl2r]U[Ssp][fl2r]U[Ssp][fl2 | UUUGGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mA[Ssp]mG[Ssp][fl2r]U[Ssp][fl2r]U[Ssp][fl2r]C[Ssp][fl2r]C[Ssp][fl2r]U[Ssp][fl2r]G[Ssp][fl2r] | CAGUUCCUGGA |
| 0105595 | G[Ssp][fl2r]A[Ssp][fl2r]G[Ssp][fl2r]C[Ssp][fl2r]U[Ssp][fl2r]U[Ssp][fl2r]U[Ssp][fl2r]U[Ssp][f]2r]U[Ssp][fl2 | GCUUUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mC[Ssp]mC[Ssp][fl2r]A[Ssp][fl2r]G[Ssp][fl2r]U[Ssp][fl2r]U[Ssp][fl2r]C[Ssp][fl2r]C[Ssp][fl2r] | GCCAGUUCCUG |
| 0105596 | U[Ssp][fl2r]G[Ssp][fl2r]G[Ssp][fl2r]A[Ssp][fl2r]G[Ssp][fl2r]C[Ssp][fl2r]U[Ssp][fl2r]U[Ssp][f]2r]U[Ssp][fl2 | GAGCUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mU[Ssp]mG[Ssp][fl2r]C[Ssp][fl2r]C[Ssp][fl2r]A[Ssp][fl2r]G[Ssp][fl2r]U[Ssp][fl2r]U[Ssp][fl2r] | CUGCCAGUUCC |
| 0105597 | C[Ssp][fl2r]C[Ssp][fl2r]U[Ssp][fl2r]G[Ssp][fl2r]G[Ssp][fl2r]A[Ssp][fl2r]G[Ssp][fl2r]U[Ssp][fl2r]U[Ssp][fl2 | UGGAGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mU[Ssp]mC[Ssp][fl2r]U[Ssp][fl2r]G[Ssp][fl2r]C[Ssp][fl2r]C[Ssp][fl2r]A[Ssp][fl2r]G[Ssp][fl2r] | CUCUGCCAGUU |
| 0105598 | U[Ssp][fl2r]U[Ssp][fl2r]C[Ssp][fl2r]C[Ssp][fl2r]U[Ssp][fl2r]G[Ssp][fl2r]G[Ssp][fl2r]U[Ssp][fl2r]U[Ssp][fl2 | CCUGGUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mC[Ssp]mC[Ssp][fl2r]U[Ssp][fl2r]C[Ssp][fl2r]U[Ssp][fl2r]G[Ssp][fl2r]C[Ssp][fl2r]C[Ssp][fl2r] | GCCUCUGCCAG |
| 0105599 | A[Ssp][fl2r]G[Ssp][fl2r]U[Ssp][fl2r]U[Ssp][fl2r]C[Ssp][fl2r]C[Ssp][fl2r]U[Ssp][fl2r]U[Ssp][fl2r]U[Ssp][f12 | UUCCUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mG[Ssp]mG[Ssp][fl2r]C[Ssp][fl2r]C[Ssp][fl2r]U[Ssp][fl2r]C[Ssp][fl2r]U[Ssp][fl2r]G[Ssp][fl2r] | CGGCCUCUGCC |
| 0105600 | C[Ssp][fl2r]C[Ssp][fl2r]A[Ssp][fl2r]G[Ssp][fl2r]U[Ssp][fl2r]U[Ssp][fl2r]C[Ssp][fl2r]U[Ssp][fl2r]U[Ssp][fl2 | AGUUCUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[f] | CUC[3nU]CGCCG |
| | 2r]C[Ssp]mC[n001R]mG | |
| ADR- | mC[n001R]mC[Ssp]mC[Ssp][fl2r]G[Ssp][fl2r]G[Ssp][fl2r]C[Ssp][fl2r]C[Ssp][fl2r]U[Ssp][fl2r]C[Ssp][fl2r] | CCCGGCCUCUG |
| 0105601 | U[Ssp][fl2r]G[Ssp][fl2r]C[Ssp][fl2r]C[Ssp][fl2r]A[Ssp][fl2r]G[Ssp][fl2r]U[Ssp][fl2r]U[Ssp][fl2r]U[Ssp][fl2 | CCAGUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[f] | CUC[3nU]CGCCG |
| | 2r]C[Ssp]mC[n001R]mG | |
| ADR- | mU[n001R]mC[Ssp]mC[Ssp][fl2r]C[Ssp][fl2r]C[Ssp][fl2r]G[Ssp][fl2r]G[Ssp][fl2r]C[Ssp][fl2r]C[Ssp][fl2r] | UCCCCGGCCUC |
| 0105602 | U[Ssp][fl2r]C[Ssp][fl2r]U[Ssp][fl2r]G[Ssp][fl2r]C[Ssp][fl2r]C[Ssp][fl2r]A[Ssp][fl2r]U[Ssp][fl2r]U[Ssp][f12 | UGCCAUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mG[Ssp]mU[Ssp][fl2r]C[Ssp][fl2r]C[Ssp][fl2r]C[Ssp][fl2r]C[Ssp][fl2r]G[Ssp][fl2r]G[Ssp][fl2r] | CGUCCCCGGCC |
| 0105603 | C[Ssp][fl2r]C[Ssp][fl2r]U[Ssp][fl2r]C[Ssp][fl2r]U[Ssp][fl2r]G[Ssp][fl2r]C[Ssp][fl2r]U[Ssp][fl2r]U[Ssp][fl2r | UCUGCUUUCUG |
| | ]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mG[Ssp]mC[Ssp][fl2r]G[Ssp][fl2r]U[Ssp][fl2r]C[Ssp][fl2r]C[Ssp][fl2r]C[Ssp][fl2r]C[Ssp][fl2r] | GGCGUCCCCGG |
| 0105604 | G[Ssp][fl2r]G[Ssp][fl2r]C[Ssp][f]2r]C[Ssp][f]2r]U[Ssp][fl2r]C[Ssp][fl2r]U[Ssp][fl2r]U[Ssp][fl2r]U[Ssp][fl2 | CCUCUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mG[Ssp]mG[Ssp][fl2r]G[Ssp][fl2r]C[Ssp][fl2r]G[Ssp][fl2r]U[Ssp][fl2r]C[Ssp][fl2r]C[Ssp][fl2r] | GGGGCGUCCCC |
| 0105605 | C[Ssp][fl2r]C[Ssp][fl2r]G[Ssp][fl2r]G[Ssp][fl2r]C[Ssp][fl2r]C[Ssp][fl2r]U[Ssp][fl2r]U[Ssp][fl2r]U[Ssp][fl2r | GGCCUUUUCUG |
| | ]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mU[Ssp]mG[Ssp][fl2r]G[Ssp][fl2r]G[Ssp][fl2r]G[Ssp][fl2r]C[Ssp][fl2r]G[Ssp][fl2r]U[Ssp][fl2r] | UUGGGGCGUCC |
| 0105606 | C[Ssp][fl2r]C[Ssp][fl2r]C[Ssp][fl2r]C[Ssp][fl2r]G[Ssp][fl2r]G[Ssp][fl2r]C[Ssp][fl2r]U[Ssp][fl2r]U[Ssp][fl2r | CCGGCUUUCUG |
| | ]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mC[Ssp]mC[Ssp][fl2r]U[Ssp][fl2r]U[Ssp][fl2r]U[Ssp][fl2r]G[Ssp][fl2r]G[Ssp][fl2r]G[Ssp][fl2r] | CCCUUUGGGGC |
| 0105608 | G[Ssp][fl2r]C[Ssp][fl2r]G[Ssp][fl2r]U[Ssp][fl2r]C[Ssp][fl2r]C[Ssp][fl2r]C[Ssp][fl2r]U[Ssp][fl2r]U[Ssp][fl2r | GUCCCUUUCUG |
| | ]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mG[Ssp]mG[Ssp][fl2r]U[Ssp][fl2r]G[Ssp][fl2r]C[Ssp][fl2r]C[Ssp][fl2r]G[Ssp][fl2r]C[Ssp][fl2r] | UGGUGCCGCUC |
| 0105609 | U[Ssp][fl2r]C[Ssp][fl2r]C[Ssp][fl2r]C[Ssp][fl2r]U[Ssp][fl2r]U[Ssp][fl2r]U[Ssp][fl2r]U[Ssp][fl2r]U[Ssp][fl2 | CCUUUUUUCUG |
| | r]U[Ssp][fl2r]C[Ssp][f]2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mU[Ssp]mG[Ssp][fl2r]G[Ssp][fl2r]G[Ssp][fl2r]U[Ssp][fl2r]C[Ssp][fl2r]U[Ssp][fl2r]C[Ssp][fl2r] | UUGGGUCUCGU |
| 0105610 | G[Ssp][fl2r]U[Ssp][fl2r]G[Ssp][fl2r]G[Ssp][fl2r]U[Ssp][fl2r]G[Ssp][fl2r]C[Ssp][fl2r]U[Ssp][fl2r]U[Ssp][f12 | GGUGCUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[f] | CUC[3nU]CGCCG |
| | 2r]C[Ssp]mC[n001R]mG | |
| ADR- | mC[n001R]mG[Ssp]mU[Ssp][fl2r]G[Ssp][fl2r]G[Ssp][fl2r]C[Ssp][fl2r]C[Ssp][fl2r]G[Ssp][fl2r]C[Ssp][fl2r] | CGUGGCCGCCU |
| 0105611 | C[Ssp][fl2r]U[Ssp][fl2r]U[Ssp][fl2r]G[Ssp][fl2r]G[Ssp][fl2r]G[Ssp][fl2r]U[Ssp][fl2r]U[Ssp][fl2r]U[Ssp][f12 | UGGGUUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[f] | CUC[3nU]CGCCG |
| | 2r]C[Ssp]mC[n001R]mG | |
| ADR- | mC[n001R]mA[Ssp]mC[Ssp][fl2r]C[Ssp][fl2r]C[Ssp][fl2r]U[Ssp][fl2r]C[Ssp][fl2r]U[Ssp][fl2r]G[Ssp][fl2r] | CACCCUCUGAC |
| 0105612 | A[Ssp][fl2r]C[Ssp][fl2r]G[Ssp][fl2r]U[Ssp][fl2r]G[Ssp][fl2r]G[Ssp][fl2r]C[Ssp][fl2r]U[Ssp][fl2r]U[Ssp][f12 | GUGGCUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mU[n001R]mU[Ssp]mC[Ssp][fl2r]A[Ssp][fl2r]C[Ssp][fl2r]C[Ssp][fl2r]U[Ssp][fl2r]G[Ssp][fl2r]C[Ssp][fl2r] | UUCACCUGCAC |
| 0105613 | A[Ssp][fl2r]C[Ssp][fl2r]A[Ssp][fl2r]C[Ssp][fl2r]C[Ssp][fl2r]C[Ssp][fl2r]U[Ssp][fl2r]U[Ssp][fl2r]U[Ssp][fl2r | ACCCUUUUCUG |
| | ]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mC[n001R]mA[Ssp]mG[Ssp][fl2r]G[Ssp][fl2r]A[Ssp][fl2r]C[Ssp][fl2r]C[Ssp][fl2r]C[Ssp][fl2r]U[Ssp][fl2r] | CAGGACCCUUU |
| 0105614 | U[Ssp][fl2r]U[Ssp][fl2r]U[Ssp][f]2r]C[Ssp][fl2r]A[Ssp][fl2r]C[Ssp][fl2r]C[Ssp][f]2r]U[Ssp][fl2r]U[Ssp][fl2 | UCACCUUUCUG |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | CUC[3nU]CGCCG |
| | 2r]C[Ssp]mC[n001R]mG | |
| ADR- | mG[n001R]mG[Ssp]mG[Ssp][fl2r]C[Ssp][fl2r]U[Ssp][fl2r]U[Ssp][fl2r]C[Ssp][fl2r]U[Ssp][fl2r]U[Ssp][fl2r] | GGGCUUCUUAG |
| 0105615 | A[Ssp][fl2r]G[Ssp][fl2r]G[Ssp][fl2r]U[Ssp][fl2r]G[Ssp][fl2r]G[Ssp][fl2r]U[Ssp][fl2r]U[Ssp][fl2r]U[Ssp][f12 | GUGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mU[Ssp]mG[Ssp][fl2r]G[Ssp][fl2r]G[Ssp][fl2r]C[Ssp][fl2r]U[Ssp][fl2r]U[Ssp][fl2r]C[Ssp][fl2r] | UUGGGCUUCUU |
| 0105616 | U[Ssp][fl2r]U[Ssp][fl2r]A[Ssp][fl2r]G[Ssp][fl2r]G[Ssp][fl2r]U[Ssp][fl2r]U[Ssp][fl2r]U[Ssp][fl2r]U[Ssp][f12 | AGGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mA[n001R]mU[Ssp]mU[Ssp][fl2r]U[Ssp][fl2r]G[Ssp][fl2r]G[Ssp][fl2r]G[Ssp][fl2r]C[Ssp][fl2r]U[Ssp][fl2r] | AUUUGGGCUUC |
| 0105617 | U[Ssp][fl2r]C[Ssp][fl2r]U[Ssp][fl2r]U[Ssp][fl2r]A[Ssp][fl2r]G[Ssp][fl2r]U[Ssp][fl2r]U[Ssp][fl2r]U[Ssp][f12 | UUAGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mA[n001R]mG[Ssp]mA[Ssp][fl2r]U[Ssp][fl2r]U[Ssp][fl2r]U[Ssp][fl2r]G[Ssp][fl2r]G[Ssp][fl2r]G[Ssp][fl2r] | AGAUUUGGGCU |
| 0105618 | C[Ssp][fl2r]U[Ssp][fl2r]U[Ssp][fl2r]C[Ssp][fl2r]U[Ssp][fl2r]U[Ssp][fl2r]U[Ssp][fl2r]U[Ssp][fl2r]U[Ssp][f12 | UCUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0105619 | G[Ssp][fl2r]G[Ssp][fl2r]C[Ssp][fl2r]U[Ssp][fl2r]U[Ssp][fl2r]C[Ssp][fl2r]U[Ssp][fl2r]U[Ssp][fl2r]U[Ssp][fl2 | CUUCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mG[Ssp]mG[Ssp][fl2r]G[Ssp][fl2r]A[Ssp][fl2r]G[Ssp][fl2r]A[Ssp][fl2r]U[Ssp][fl2r]U[Ssp][fl2r] | UGGGAGAUUUG |
| 0105620 | U[Ssp][fl2r]G[Ssp][fl2r]G[Ssp][fl2r]G[Ssp][fl2r]C[Ssp][fl2r]U[Ssp][fl2r]U[Ssp][fl2r]U[Ssp][fl2r]U[Ssp][fl2 | GGCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mU[Ssp]mU[Ssp][fl2r]G[Ssp][fl2r]G[Ssp][fl2r]G[Ssp][fl2r]A[Ssp][fl2r]G[Ssp][fl2r]A[Ssp][fl2r] | UUUGGGAGAUU |
| 0105621 | U[Ssp][fl2r]U[Ssp][fl2r]U[Ssp][fl2r]G[Ssp][fl2r]G[Ssp][fl2r]G[Ssp][fl2r]U[Ssp][fl2r]U[Ssp][fl2r]U[Ssp][fl2 | UGGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mC[Ssp]mU[Ssp][fl2r]U[Ssp][fl2r]U[Ssp][fl2r]G[Ssp][fl2r]G[Ssp][fl2r]G[Ssp][fl2r]A[Ssp][fl2r] | GCUUUGGGAGA |
| 0105622 | G[Ssp][fl2r]A[Ssp][fl2r]U[Ssp][fl2r]U[Ssp][fl2r]U[Ssp][fl2r]G[Ssp][fl2r]U[Ssp][fl2r]U[Ssp][fl2r]U[Ssp][fl2 | UUUGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0105623 | G[Ssp][fl2r]A[Ssp][fl2r]G[Ssp][fl2r]A[Ssp][fl2r]U[Ssp][fl2r]U[Ssp][fl2r]U[Ssp][fl2r]U[Ssp][fl2r]U[Ssp][fl2 | GAUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mG[Ssp]mG[Ssp][fl2r]A[Ssp][fl2r]G[Ssp][fl2r]C[Ssp][fl2r]U[Ssp][fl2r]U[Ssp][fl2r]U[Ssp][fl2r] | UGGAGCUUUGG |
| 0105624 | G[Ssp][fl2r]G[Ssp][fl2r]G[Ssp][fl2r]A[Ssp][fl2r]G[Ssp][fl2r]A[Ssp][fl2r]U[Ssp][fl2r]U[Ssp][fl2r]U[Ssp][f12 | GAGAUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mA[n001R]mG[Ssp]mU[Ssp][fl2r]U[Ssp][fl2r]C[Ssp][fl2r]C[Ssp][fl2r]U[Ssp][fl2r]G[Ssp][fl2r]G[Ssp][fl2r] | AGUUCCUGGAG |
| 0105627 | A[Ssp][fl2r]G[Ssp][fl2r]C[Ssp][fl2r]U[Ssp][fl2r]U[Ssp][fl2r]U[Ssp][fl2r]U[Ssp][fl2r]U[Ssp][fl2r]U[Ssp][f12 | CUUUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mC[n001R]mC[Ssp]mA[Ssp][fl2r]G[Ssp][fl2r]U[Ssp][fl2r]U[Ssp][fl2r]C[Ssp][fl2r]C[Ssp][fl2r]U[Ssp][fl2r] | CCAGUUCCUGG |
| 0105628 | G[Ssp][fl2r]G[Ssp][fl2r]A[Ssp][fl2r]G[Ssp][fl2r]C[Ssp][fl2r]U[Ssp][fl2r]U[Ssp][fl2r]U[Ssp][f]2r]U[Ssp][fl2 | AGCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mG[Ssp]mC[Ssp][fl2r]C[Ssp][fl2r]A[Ssp][fl2r]G[Ssp][fl2r]U[Ssp][fl2r]U[Ssp][fl2r]C[Ssp][fl2r] | UGCCAGUUCCU |
| 0105629 | C[Ssp][fl2r]U[Ssp][fl2r]G[Ssp][fl2r]G[Ssp][fl2r]A[Ssp][fl2r]G[Ssp][fl2r]U[Ssp][fl2r]U[Ssp][f]2r]U[Ssp][f12 | GGAGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mC[Ssp]mU[Ssp][fl2r]G[Ssp][fl2r]C[Ssp][fl2r]C[Ssp][fl2r]A[Ssp][fl2r]G[Ssp][fl2r]U[Ssp][fl2r] | UCUGCCAGUUC |
| 0105630 | U[Ssp][fl2r]C[Ssp][fl2r]C[Ssp][fl2r]U[Ssp][fl2r]G[Ssp][fl2r]G[Ssp][fl2r]U[Ssp][fl2r]U[Ssp][fl2r]U[Ssp][fl2 | CUGGUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mC[n001R]mC[Ssp]mU[Ssp][fl2r]C[Ssp][fl2r]U[Ssp][fl2r]G[Ssp][fl2r]C[Ssp][fl2r]C[Ssp][f]2r]A[Ssp][fl2r] | CCUCUGCCAGU |
| 0105631 | G[Ssp][fl2r]U[Ssp][fl2r]U[Ssp][fl2r]C[Ssp][fl2r]C[Ssp][fl2r]U[Ssp][fl2r]U[Ssp][fl2r]U[Ssp][fl2r]U[Ssp][f12 | UCCUUUUCUGC |
| | r]C[Ssp][2][Ssp][2][Ssp][2][Ssp][fl2][Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mG[Ssp]mC[Ssp][fl2r]C[Ssp][fl2r]U[Ssp][fl2r]C[Ssp][fl2r]U[Ssp][fl2r]G[Ssp][fl2r]C[Ssp][fl2r] | GGCCUCUGCCA |
| 0105632 | C[Ssp][fl2r]A[Ssp][fl2r]G[Ssp][fl2r]U[Ssp][fl2r]U[Ssp][fl2r]C[Ssp][fl2r]U[Ssp][fl2r]U[Ssp][fl2r]U[Ssp][f12 | GUUCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mC[n001R]mC[Ssp]mG[Ssp][f]2r]G[Ssp][fl2r]C[Ssp][fl2r]C[Ssp][fl2r]U[Ssp][fl2r]C[Ssp][fl2r]U[Ssp][fl2r] | CCGGCCUCUGC |
| 0105633 | G[Ssp][fl2r]C[Ssp][fl2r]C[Ssp][fl2r]A[Ssp][fl2r]G[Ssp][fl2r]U[Ssp][fl2r]U[Ssp][fl2r]U[Ssp][fl2r]U[Ssp][f12 | CAGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0105634 | C[Ssp][fl2r]U[Ssp][fl2r]G[Ssp][fl2r]C[Ssp][fl2r]C[Ssp][fl2r]A[Ssp][fl2r]U[Ssp][fl2r]U[Ssp][fl2r]U[Ssp][fl2 | GCCAUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mU[Ssp]mC[Ssp][fl2r]C[Ssp][fl2r]C[Ssp][fl2r]C[Ssp][fl2r]G[Ssp][fl2r]G[Ssp][fl2r]C[Ssp][fl2r] | GUCCCCGGCCU |
| 0105635 | C[Ssp][fl2r]U[Ssp][fl2r]C[Ssp][fl2r]U[Ssp][fl2r]G[Ssp][fl2r]C[Ssp][fl2r]U[Ssp][fl2r]U[Ssp][fl2r]U[Ssp][fl2 | CUGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mC[Ssp]mG[Ssp][fl2r]U[Ssp][fl2r]C[Ssp][fl2r]C[Ssp][fl2r]C[Ssp][fl2r]C[Ssp][fl2r]G[Ssp][fl2r] | GCGUCCCCGGC |
| 0105636 | G[Ssp][fl2r]C[Ssp][fl2r]C[Ssp][fl2r]U[Ssp][fl2r]C[Ssp][fl2r]U[Ssp][fl2r]U[Ssp][fl2r]U[Ssp][fl2r]U[Ssp][fl2 | CUCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mG[Ssp]mG[Ssp][fl2r]C[Ssp][fl2r]G[Ssp][fl2r]U[Ssp][fl2r]C[Ssp][fl2r]C[Ssp][fl2r]C[Ssp][fl2r] | GGGCGUCCCCG |
| 0105637 | C[Ssp][fl2r]G[Ssp][fl2r]G[Ssp][fl2r]C[Ssp][fl2r]C[Ssp][fl2r]U[Ssp][fl2r]U[Ssp][fl2r]U[Ssp][fl2r]U[Ssp][fl2 | GCCUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mG[Ssp]mG[Ssp][fl2r]G[Ssp][fl2r]G[Ssp][fl2r]C[Ssp][fl2r]G[Ssp][fl2r]U[Ssp][fl2r]C[Ssp][fl2r] | UGGGGCGUCCC |
| 0105638 | C[Ssp][fl2r]C[Ssp][fl2r]C[Ssp][fl2r]G[Ssp][fl2r]G[Ssp][fl2r]C[Ssp][fl2r]U[Ssp][fl2r]U[Ssp][fl2r]U[Ssp][fl2r | CGGCUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCGG |
| | [Ssp]mG[n001R]mG | |
| ADR- | mC[n001R]mC[Ssp]mU[Ssp][fl2r]U[Ssp][fl2r]U[Ssp][fl2r]G[Ssp][fl2r]G[Ssp][fl2r]G[Ssp][fl2r]G[Ssp][fl2r] | CCUUUGGGGCG |
| 0105640 | C[Ssp][fl2r]G[Ssp][fl2r]U[Ssp][fl2r]C[Ssp][fl2r]C[Ssp][fl2r]C[Ssp][fl2r]U[Ssp][fl2r]U[Ssp][fl2r]U[Ssp][fl2r | UCCCUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCGG |
| | [Ssp]mG[n001R]mG | |
| ADR- | mG[n001R]mG[Ssp]mU[Ssp][fl2r]G[Ssp][fl2r]C[Ssp][fl2r]C[Ssp][fl2r]G[Ssp][fl2r]C[Ssp][fl2r]U[Ssp][fl2r] | GGUGCCGCUCC |
| 0105641 | C[Ssp][fl2r]C[Ssp][fl2r]C[Ssp][fl2r]U[Ssp][f]2r]U[Ssp][fl2r]U[Ssp][fl2r]U[Ssp][fl2r]U[Ssp][fl2r]U[Ssp][fl2 | CUUUUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCGG |
| | C[Ssp]mG[n001R]mG | |
| ADR- | mU[n001R]mG[Ssp]mG[Ssp][fl2r]G[Ssp][fl2r]U[Ssp][fl2r]C[Ssp][fl2r]U[Ssp][fl2r]C[Ssp][fl2r]G[Ssp][fl2r] | UGGGUCUCGUG |
| 0105642 | U[Ssp][fl2r]G[Ssp][fl2r]G[Ssp][fl2r]U[Ssp][fl2r]G[Ssp][fl2r]C[Ssp][fl2r]U[Ssp][fl2r]U[Ssp][fl2r]U[Ssp][f12 | GUGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mG[n001R]mU[Ssp]mG[Ssp][fl2r]G[Ssp][fl2r]C[Ssp][fl2r]C[Ssp][fl2r]G[Ssp][fl2r]C[Ssp][fl2r]C[Ssp][fl2r] | GUGGCCGCCUU |
| 0105643 | U[Ssp][fl2r]U[Ssp][fl2r]G[Ssp][fl2r]G[Ssp][fl2r]G[Ssp][fl2r]U[Ssp][fl2r]U[Ssp][fl2r]U[Ssp][fl2r]U[Ssp][fl2 | GGGUUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mA[n001R]mC[Ssp]mC[Ssp][fl2r]C[Ssp][fl2r]U[Ssp][fl2r]C[Ssp][fl2r]U[Ssp][fl2r]G[Ssp][fl2r]A[Ssp][fl2r] | ACCCUCUGACG |
| 0105644 | C[Ssp][fl2r]G[Ssp][fl2r]U[Ssp][f]2r]G[Ssp][fl2r]G[Ssp][fl2r]C[Ssp][fl2r]U[Ssp][fl2r]U[Ssp][f]2r]U[Ssp][fl2 | UGGCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mU[n001R]mC[Ssp]mA[Ssp][fl2r]C[Ssp][fl2r]C[Ssp][fl2r]U[Ssp][fl2r]G[Ssp][fl2r]C[Ssp][fl2r]A[Ssp][fl2r] | UCACCUGCACA |
| 0105645 | C[Ssp][fl2r]A[Ssp][fl2r]C[Ssp][fl2r]C[Ssp][fl2r]C[Ssp][fl2r]U[Ssp][fl2r]U[Ssp][fl2r]U[Ssp][fl2r]U[Ssp][fl2r | CCCUUUUCUGC |
| | ]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]mC | UC[3nU]CGCCGG |
| | [Ssp]mG[n001R]mG | |
| ADR- | mA[n001R]mG[Ssp]mG[Ssp][fl2r]A[Ssp][fl2r]C[Ssp][fl2r]C[Ssp][fl2r]C[Ssp][fl2r]U[Ssp][fl2r]U[Ssp][fl2r] | AGGACCCUUUU |
| 0105646 | U[Ssp][fl2r]U[Ssp][fl2r]C[Ssp][fl2r]A[Ssp][fl2r]C[Ssp][fl2r]C[Ssp][fl2r]U[Ssp][fl2r]U[Ssp][fl2r]U[Ssp][f12 | CACCUUUCUGC |
| | r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2r]C[Ssp]m | UC[3nU]CGCCGG |
| | C[Ssp]mG[n001R]mG | |
| ADR- | mC[n001R]mU[Ssp]mC[Ssp][fl2r]G[Ssp][fl2r]A[Ssp][fl2r]C[Ssp][fl2r]U[Ssp][fl2r]U[Ssp][fl2r]A[Ssp][fl2r] | CUCGACUUACU |
| 0106651 | C[Ssp][f]2r]U[Ssp][fl2r]C[Ssp][fl2r]C[Ssp][fl2r]A[Ssp][fl2r]A[Ssp][fl2r]U[Ssp][fl2r]C[Ssp][fl2r]U[Ssp][fl2r | CCAAUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mC[Ssp][fl2r]U[Ssp][fl2r]C[Ssp][fl2r]G[Ssp][fl2r]A[Ssp][fl2r]C[Ssp][fl2r]U[Ssp][fl2r] | UUCUCGACUUA |
| 0106652 | U[Ssp][fl2r]A[Ssp][fl2r]C[Ssp][fl2r]U[Ssp][fl2r]C[Ssp][fl2r]C[Ssp][fl2r]U[Ssp][fl2r]C[Ssp][fl2r]U[Ssp][fl2r | CUCCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mC[Ssp]mU[Ssp][fl2r]U[Ssp][fl2r]C[Ssp][fl2r]U[Ssp][fl2r]C[Ssp][fl2r]G[Ssp][fl2r]A[Ssp][fl2r] | ACUUCUCGACU |
| 0106653 | C[Ssp][fl2r]U[Ssp][fl2r]U[Ssp][fl2r]A[Ssp][fl2r]C[Ssp][fl2r]U[Ssp][fl2r]U[Ssp][fl2r]C[Ssp][fl2r]U[Ssp][fl2 | UACUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mU[Ssp]mA[Ssp][fl2r]C[Ssp][fl2r]U[Ssp][fl2r]U[Ssp][fl2r]C[Ssp][fl2r]U[Ssp][fl2r]C[Ssp][fl2r] | AUACUUCUCGA |
| 0106654 | G[Ssp][fl2r]A[Ssp][fl2r]C[Ssp][fl2r]U[Ssp][fl2r]U[Ssp][fl2r]A[Ssp][fl2r]U[Ssp][fl2r]C[Ssp][fl2r]U[Ssp][f12 | CUUAUCUAUAU |
| | r]A[S][2][Ssp][fl2]A[Ssp][2][Ssp][fl2][Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mA[Ssp]mA[Ssp][fl2r]U[Ssp][fl2r]A[Ssp][fl2r]C[Ssp][fl2r]U[Ssp][fl2r]U[Ssp][fl2r]C[Ssp][fl2r] | AAAUACUUCUC |
| 0106655 | U[Ssp][fl2r]C[Ssp][fl2r]G[Ssp][fl2r]A[Ssp][fl2r]C[Ssp][fl2r]U[Ssp][fl2r]U[Ssp][fl2r]C[Ssp][fl2r]U[Ssp][f12 | GACUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mC[Ssp]mA[Ssp][fl2r]A[Ssp][fl2r]A[Ssp][fl2r]U[Ssp][fl2r]A[Ssp][fl2r]C[Ssp][fl2r]U[Ssp][fl2r] | UCAAAUACUUC |
| 0106656 | U[Ssp][fl2r]C[Ssp][fl2r]U[Ssp][fl2r]C[Ssp][fl2r]G[Ssp][fl2r]A[Ssp][fl2r]U[Ssp][fl2r]C[Ssp][fl2r]U[Ssp][f12 | UCGAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mG[Ssp]mU[Ssp][fl2r]C[Ssp][fl2r]A[Ssp][fl2r]A[Ssp][fl2r]A[Ssp][fl2r]U[Ssp][fl2r]A[Ssp][fl2r] | AGUCAAAUACU |
| 0106657 | C[Ssp][fl2r]U[Ssp][fl2r]U[Ssp][fl2r]C[Ssp][f]2r]U[Ssp][fl2r]C[Ssp][fl2r]U[Ssp][fl2r]C[Ssp][fl2r]U[Ssp][fl2r | UCUCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mA[Ssp]mA[Ssp][fl2r]G[Ssp][fl2r]U[Ssp][fl2r]C[Ssp][fl2r]A[Ssp][fl2r]A[Ssp][fl2r]A[Ssp][fl2r] | GAAGUCAAAUA |
| 0106658 | U[Ssp][fl2r]A[Ssp][fl2r]C[Ssp][fl2r]U[Ssp][fl2r]U[Ssp][fl2r]C[Ssp][fl2r]U[Ssp][fl2r]C[Ssp][fl2r]U[Ssp][fl2 | CUUCUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mG[Ssp][fl2r]A[Ssp][fl2r]A[Ssp][fl2r]G[Ssp][fl2r]U[Ssp][fl2r]C[Ssp][fl2r]A[Ssp][fl2r] | CUGAAGUCAAA |
| 0106659 | A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2r]U[Ssp][fl2 | UACUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mA[Ssp]mC[Ssp][fl2r]U[Ssp][fl2r]G[Ssp][fl2r]A[Ssp][fl2r]A[Ssp][fl2r]G[Ssp][fl2r]U[Ssp][fl2r] | GACUGAAGUCA |
| 0106660 | C[Ssp][fl2r]A[Ssp][fl2r]A[Ssp][fl2r]A[Ssp][fl2r]U[Ssp][fl2r]A[Ssp][fl2r]U[Ssp][fl2r]C[Ssp][fl2r]U[Ssp][fl2 | AAUAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mG[Ssp][fl2r]A[Ssp][fl2r]C[Ssp][fl2r]U[Ssp][fl2r]G[Ssp][fl2r]A[Ssp][fl2r]A[Ssp][fl2r] | CUGACUGAAGU |
| 0106661 | G[Ssp][fl2r]U[Ssp][fl2r]C[Ssp][fl2r]A[Ssp][fl2r]A[Ssp][fl2r]A[Ssp][fl2r]U[Ssp][fl2r]C[Ssp][fl2r]U[Ssp][fl2 | CAAAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mG[Ssp]mC[Ssp][fl2r]U[Ssp][fl2r]G[Ssp][fl2r]A[Ssp][fl2r]C[Ssp][fl2r]U[Ssp][fl2r]G[Ssp][fl2r] | CGCUGACUGAA |
| 0106662 | A[Ssp][fl2r]A[Ssp][fl2r]G[Ssp][fl2r]U[Ssp][fl2r]C[Ssp][fl2r]A[Ssp][fl2r]U[Ssp][fl2r]C[Ssp][fl2r]U[Ssp][fl2 | GUCAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mU[Ssp]mC[Ssp][fl2r]G[Ssp][fl2r]C[Ssp][fl2r]U[Ssp][fl2r]G[Ssp][fl2r]A[Ssp][fl2r]C[Ssp][fl2r] | GUCGCUGACUG |
| 0106663 | U[Ssp][fl2r]G[Ssp][fl2r]A[Ssp][fl2r]A[Ssp][fl2r]G[Ssp][fl2r]U[Ssp][fl2r]U[Ssp][fl2r]C[Ssp][fl2r]U[Ssp][fl2 | AAGUUCUAUAU |
| | r]A[Ssp][fl2][Ssp][fl2]A[Ssp][fl2r]U[Ssp][fl2r][Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mC[Ssp]mG[Ssp][fl2r]U[Ssp][fl2r]C[Ssp][fl2r]G[Ssp][fl2r]C[Ssp][fl2r]U[Ssp][fl2r]G[Ssp][fl2r] | CCGUCGCUGAC |
| 0106664 | A[Ssp][fl2r]C[Ssp][fl2r]U[Ssp][fl2r]G[Ssp][fl2r]A[Ssp][fl2r]A[Ssp][fl2r]U[Ssp][fl2r]C[Ssp][fl2r]U[Ssp][f12 | UGAAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mC[Ssp][fl2r]C[Ssp][fl2r]G[Ssp][fl2r]U[Ssp][fl2r]C[Ssp][fl2r]G[Ssp][fl2r]C[Ssp][fl2r] | UUCCGUCGCUG |
| 0106665 | U[Ssp][fl2r]G[Ssp][fl2r]A[Ssp][fl2r]C[Ssp][fl2r]U[Ssp][fl2r]G[Ssp][fl2r]U[Ssp][fl2r]C[Ssp][fl2r]U[Ssp][fl2 | ACUGUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mU[Ssp][fl2r]U[Ssp][fl2r]C[Ssp][fl2r]C[Ssp][fl2r]G[Ssp][fl2r]U[Ssp][fl2r]C[Ssp][fl2r] | CUUUCCGUCGC |
| 0106666 | G[Ssp][fl2r]C[Ssp][fl2r]U[Ssp][fl2r]G[Ssp][fl2r]A[Ssp][fl2r]C[Ssp][fl2r]U[Ssp][fl2r]C[Ssp][fl2r]U[Ssp][fl2 | UGACUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mC[Ssp][fl2r]U[Ssp][fl2r]U[Ssp][fl2r]U[Ssp][fl2r]C[Ssp][fl2r]C[Ssp][fl2r]G[Ssp][fl2r] | CUCUUUCCGUC |
| 0106667 | U[Ssp][fl2r]C[Ssp][fl2r]G[Ssp][fl2r]C[Ssp][fl2r]U[Ssp][fl2r]G[Ssp][fl2r]U[Ssp][fl2r]C[Ssp][fl2r]U[Ssp][fl2 | GCUGUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mA[Ssp]mC[Ssp][fl2r]U[Ssp][fl2r]C[Ssp][fl2r]U[Ssp][fl2r]U[Ssp][fl2r]U[Ssp][fl2r]C[Ssp][fl2r] | UACUCUUUCCG |
| 0106668 | C[Ssp][fl2r]G[Ssp][fl2r]U[Ssp][fl2r]C[Ssp][fl2r]G[Ssp][fl2r]C[Ssp][fl2r]U[Ssp][fl2r]C[Ssp][fl2r]U[Ssp][fl2r | UCGCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mA[Ssp]mU[Ssp][fl2r]A[Ssp][fl2r]C[Ssp][fl2r]U[Ssp][fl2r]C[Ssp][fl2r]U[Ssp][fl2r]U[Ssp][fl2r] | CAUACUCUUUC |
| 0106669 | U[Ssp][fl2r]C[Ssp][fl2r]C[Ssp][fl2r]G[Ssp][fl2r]U[Ssp][fl2r]C[Ssp][fl2r]U[Ssp][fl2r]C[Ssp][fl2r]U[Ssp][fl2r | CGUCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mC[Ssp][fl2r]A[Ssp][fl2r]U[Ssp][fl2r]A[Ssp][fl2r]C[Ssp][fl2r]U[Ssp][fl2r]C[Ssp][fl2r] | CUCAUACUCUU |
| 0106670 | U[Ssp][fl2r]U[Ssp][fl2r]U[Ssp][fl2r]C[Ssp][fl2r]C[Ssp][fl2r]G[Ssp][fl2r]U[Ssp][fl2r]C[Ssp][fl2r]U[Ssp][fl2 | UCCGUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mG[Ssp]mC[Ssp][fl2r]U[Ssp][fl2r]C[Ssp][fl2r]A[Ssp][fl2r]U[Ssp][fl2r]A[Ssp][fl2r]C[Ssp][fl2r] | AGCUCAUACUC |
| 0106671 | U[Ssp][fl2r]C[Ssp][fl2r]U[Ssp][fl2r]U[Ssp][fl2r]U[Ssp][fl2r]C[Ssp][fl2r]U[Ssp][fl2r]C[Ssp][fl2r]U[Ssp][fl2 | UUUCUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mC[Ssp]mA[Ssp][fl2r]G[Ssp][fl2r]C[Ssp][fl2r]U[Ssp][fl2r]C[Ssp][fl2r]A[Ssp][fl2r]U[Ssp][fl2r] | CCAGCUCAUAC |
| 0106672 | A[Ssp][fl2r]C[Ssp][fl2r]U[Ssp][fl2r]C[Ssp][fl2r]U[Ssp][fl2r]U[Ssp][fl2r]U[Ssp][fl2r]C[Ssp][fl2r]U[Ssp][fl2 | UCUUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mC[Ssp][fl2r]C[Ssp][fl2r]A[Ssp][fl2r]G[Ssp][fl2r]C[Ssp][fl2r]U[Ssp][fl2r]C[Ssp][fl2r] | UUCCAGCUCAU |
| 0106673 | A[Ssp][fl2r]U[Ssp][fl2r]A[Ssp][f]2r]C[Ssp][fl2r]U[Ssp][fl2r]C[Ssp][fl2r]U[Ssp][fl2r]C[Ssp][fl2r]U[Ssp][fl2 | ACUCUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mU[Ssp][fl2r]U[Ssp][fl2r]C[Ssp][fl2r]C[Ssp][fl2r]A[Ssp][fl2r]G[Ssp][fl2r]C[Ssp][fl2r] | UUUUCCAGCUC |
| 0106674 | U[Ssp][fl2r]C[Ssp][fl2r]A[Ssp][fl2r]U[Ssp][fl2r]A[Ssp][fl2r]C[Ssp][fl2r]U[Ssp][fl2r]C[Ssp][fl2r]U[Ssp][fl2 | AUACUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mU[Ssp]mU[Ssp][fl2r]U[Ssp][fl2r]U[Ssp][fl2r]U[Ssp][fl2r]C[Ssp][fl2r]C[Ssp][fl2r]A[Ssp][fl2r] | GUUUUUCCAGC |
| 0106675 | G[Ssp][f]2r]C[Ssp][fl2r]U[Ssp][fl2r]C[Ssp][fl2r]A[Ssp][fl2r]U[Ssp][fl2r]U[Ssp][fl2r]C[Ssp][fl2r]U[Ssp][fl2 | UCAUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mG[Ssp][fl2r]U[Ssp][fl2r]U[Ssp][fl2r]U[Ssp][fl2r]U[Ssp][fl2r]U[Ssp][fl2r]C[Ssp][fl2r] | CUGUUUUUCCA |
| 0106676 | C[Ssp][fl2r]A[Ssp][fl2r]G[Ssp][fl2r]C[Ssp][fl2r]U[Ssp][fl2r]C[Ssp][fl2r]U[Ssp][fl2r]C[Ssp][fl2r]U[Ssp][fl2r | GCUCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mC[Ssp][fl2r]U[Ssp][fl2r]G[Ssp][fl2r]U[Ssp][fl2r]U[Ssp][fl2r]U[Ssp][fl2r]U[Ssp][fl2r] | UUCUGUUUUUC |
| 0106677 | U[Ssp][fl2r]C[Ssp][fl2r]C[Ssp][fl2r]A[Ssp][fl2r]G[Ssp][fl2r]C[Ssp][fl2r]U[Ssp][fl2r]C[Ssp][fl2r]U[Ssp][fl2r | CAGCUCUAUAU |
| | ]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mU[Ssp][fl2r]U[Ssp][fl2r]C[Ssp][fl2r]U[Ssp][fl2r]G[Ssp][fl2r]U[Ssp][fl2r]U[Ssp][fl2r] | UUUUCUGUUUU |
| 0106678 | U[Ssp][fl2r]U[Ssp][fl2r]U[Ssp][fl2r]C[Ssp][fl2r]C[Ssp][fl2r]A[Ssp][fl2r]U[Ssp][fl2r]C[Ssp][fl2r]U[Ssp][fl2 | UCCAUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mU[Ssp][fl2r]U[Ssp][fl2r]U[Ssp][fl2r]U[Ssp][fl2r]C[Ssp][fl2r]U[Ssp][fl2r]G[Ssp][fl2r] | UUUUUUCUGUU |
| 0106679 | U[Ssp][fl2r]U[Ssp][fl2r]U[Ssp][fl2r]U[Ssp][fl2r]U[Ssp][fl2r]C[Ssp][fl2r]U[Ssp][fl2r]C[Ssp][fl2r]U[Ssp][fl2 | UUUCUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mG[Ssp]mU[Ssp][fl2r]U[Ssp][fl2r]U[Ssp][fl2r]U[Ssp][fl2r]U[Ssp][fl2r]U[Ssp][fl2r]C[Ssp][fl2r] | AGUUUUUUCUG |
| 0106680 | U[Ssp][fl2r]G[Ssp][fl2r]U[Ssp][fl2r]U[Ssp][fl2r]U[Ssp][fl2r]U[Ssp][fl2r]U[Ssp][fl2r]C[Ssp][fl2r]U[Ssp][f12 | UUUUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mC[Ssp][fl2r]C[Ssp][fl2r]U[Ssp][fl2r]U[Ssp][fl2r]U[Ssp][fl2r]U[Ssp][fl2r]C[Ssp][fl2r] | UUCCUUUUCAA |
| 0106681 | A[Ssp][fl2r]A[Ssp][fl2r]G[Ssp][fl2r]U[Ssp][fl2r]U[Ssp][fl2r]U[Ssp][fl2r]U[Ssp][fl2r]C[Ssp][fl2r]U[Ssp][f12 | GUUUUCUAUAU |
| | r]A[Ssp][fl2][Ssp][fl2]A[Ssp][fl2][Ssp][fl2][Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mU[Ssp]mU[Ssp][fl2r]C[Ssp][fl2r]U[Ssp][fl2r]U[Ssp][fl2r]G[Ssp][fl2r]U[Ssp][fl2r]C[Ssp][fl2r] | GUUCUUGUCUU |
| 0106682 | U[Ssp][fl2r]U[Ssp][fl2r]U[Ssp][f]2r]C[Ssp][fl2r]C[Ssp][fl2r]U[Ssp][fl2r]U[Ssp][fl2r]C[Ssp][fl2r]U[Ssp][fl2 | UCCUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mU[n001R]mU[Ssp]mU[Ssp][fl2r]U[Ssp][fl2r]G[Ssp][fl2r]G[Ssp][fl2r]A[Ssp][fl2r]G[Ssp][fl2r]U[Ssp][fl2r] | UUUUGGAGUUG |
| 0106683 | U[Ssp][fl2r]G[Ssp][fl2r]U[Ssp][fl2r]U[Ssp][fl2r]C[Ssp][fl2r]U[Ssp][fl2r]U[Ssp][fl2r]C[Ssp][fl2r]U[Ssp][fl2 | UUCUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mC[n001R]mU[Ssp]mC[Ssp][fl2r]U[Ssp][fl2r]U[Ssp][fl2r]G[Ssp][fl2r]C[Ssp][fl2r]U[Ssp][fl2r]C[Ssp][fl2r] | CUCUUGCUCCU |
| 0106684 | C[Ssp][fl2r]U[Ssp][fl2r]U[Ssp][fl2r]U[Ssp][fl2r]U[Ssp][fl2r]G[Ssp][fl2r]U[Ssp][fl2r]C[Ssp][fl2r]U[Ssp][f12 | UUUGUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mA[n001R]mA[Ssp]mA[Ssp][fl2r]A[Ssp][fl2r]G[Ssp][fl2r]G[Ssp][fl2r]C[Ssp][fl2r]U[Ssp][fl2r]U[Ssp][fl2r] | AAAAGGCUUUC |
| 0106685 | U[Ssp][fl2r]C[Ssp][fl2r]U[Ssp][fl2r]C[Ssp][fl2r]U[Ssp][fl2r]U[Ssp][fl2r]U[Ssp][fl2r]C[Ssp][fl2r]U[Ssp][fl2 | UCUUUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp]m | CT[3nU]GCCUC |
| | U[n001R]mC | |
| ADR- | mG[n001R]mG[Ssp]mG[Ssp][fl2r]A[Ssp][fl2r]G[Ssp][fl2r]A[Ssp][fl2r]A[Ssp][fl2r]A[Ssp][fl2r]U[Ssp]d[3n | GGGAGAAAU[3n |
| 0106704 | U][Ssp]dC[Ssp][fl2r]A[Ssp][fl2r]C[Ssp][fl2r]C[Ssp][fl2r]U[Ssp][fl2r]G[Ssp][fl2r]U[Ssp][fl2r]C[Ssp][fl2r]U | U]CACCUGUCU |
| | [Ssp][fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r] | AUAUCT[3nU]GC |
| | C[Ssp]mU[n001R]mC | CUC |
| ADR- | mG[n001R]mG[Ssp]mG[Ssp][fl2r]A[Ssp][fl2r]G[Ssp][fl2r]A[Ssp][fl2r]A[Ssp][fl2r]A[Ssp][fl2r]U[Ssp]d[3n | GGGAGAAAU[3n |
| 0106705 | U][Ssp]dC[Ssp][fl2r]A[Ssp][fl2r]C[Ssp][fl2r]C[Ssp][fl2r]U[Ssp][fl2r]G[Ssp][fl2r]A[Ssp][fl2r]G[Ssp][fl2r]A | U]CACCUGAGA |
| | [Ssp][fl2r]U[Ssp][fl2r]C[Ssp][fl2r]U[Ssp][fl2r]A[Ssp][fl2r]U[Ssp]dA[Ssp]d[3nU][Ssp]dC[n001R]mUp[fl2r] | UCUAUA[3nU]C |
| | U[Ssp]mG[n001R]mC | UUGC |
| ADR- | mG[n001R]mG[Ssp]mG[Ssp][fl2r]A[Ssp][fl2r]G[Ssp][fl2r]A[Ssp][fl2r]A[Ssp][fl2r]A[Ssp][fl2r]U[Ssp]d[3n | GGGAGAAAU[3n |
| 0106706 | U][Ssp]dC[Ssp][fl2r]A[Ssp][fl2r]C[Ssp][fl2r]C[Ssp][fl2r]U[Ssp][fl2r]G[Ssp][fl2r]C[Ssp][fl2r]A[Ssp][fl2r]A | U]CACCUGCAA |
| | [Ssp][fl2r]G[Ssp][fl2r]A[Ssp][fl2r]U[Ssp][fl2r]C[Ssp][fl2r]U[Ssp]dA[Ssp]d[3nU][Ssp]dA[n001R]mUp[fl2r] | GAUCUA[3nU]A |
| | C[Ssp]mU[n001R]mU | UCUU |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0106831 | C[Ssp][fl2r]U[Ssp][fl2r]C[Ssp][fl2r]U[Ssp][fl2r]C[Ssp][fl2r]A[Ssp][SpC3][Ssp][fl2r]U[Ssp][fl2r]A[Ssp][fl2 | CUCAUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0106832 | C[Ssp][fl2r]U[Ssp][fl2r]C[Ssp][fl2r]U[Ssp][fl2r]C[Ssp][fl2r]A[Ssp][oC40][Ssp][fl2r]U[Ssp][fl2r]A[Ssp][fl2 | CUCAUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0106833 | C[Ssp][fl2r]U[Ssp][fl2r]C[Ssp][fl2r]U[Ssp][fl2r]C[Ssp][fl2r]A[Ssp][oC60][Ssp][fl2r]U[Ssp][fl2r]A[Ssp][fl2 | CUCAUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0106834 | C[Ssp][fl2r]U[Ssp][fl2r]C[Ssp][fl2r]U[Ssp][fl2r]C[Ssp][fl2r]A[Ssp][Sp9][Ssp][fl2r]U[Ssp][fl2r]A[Ssp][fl2r] | CUCAUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[f12 | UT[3nU]ACUCC |
| | r]U[Ssp]mC[n001R]mC | |
| ADR- | mU[n001R]mA[Ssp]mG[Ssp][fl2r]U[Ssp][fl2r]C[Ssp][fl2r]C[Ssp][fl2r]U[Ssp][fl2r]U[Ssp][fl2r]U[Ssp][fl2r] | UAGUCCUUUCU |
| 0106835 | C[Ssp][fl2r]U[Ssp][fl2r]C[Ssp][fl2r]U[Ssp][fl2r]C[Ssp][fl2r]A[Ssp][Sp12][sp][fl2r]U[Ssp][fl2r]A[Ssp][fl2r] | CUCAUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | r]U[Ssp]mC[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0106836 | C[Ssp][fl2r]C[Ssp][fl2r]U[Ssp][fl2r]U[Ssp][fl2r]U[Ssp][fl2r]C[Ssp][SpC3][Ssp][fl2r]U[Ssp][fl2r]A[Ssp][f12 | UUUCUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0106837 | C[Ssp][fl2r]C[Ssp][fl2r]U[Ssp][fl2r]U[Ssp][fl2r]U[Ssp][fl2r]C[Ssp][oC40][Ssp][fl2r]U[Ssp][fl2r]A[Ssp][fl2 | UUUCUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0106838 | C[Ssp][fl2r]C[Ssp][fl2r]U[Ssp][fl2r]U[Ssp][fl2r]U[Ssp][fl2r]C[Ssp][oC60][Ssp][fl2r]U[Ssp][fl2r]A[Ssp][fl2 | UUUCUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0106839 | C[Ssp][fl2r]C[Ssp][fl2r]U[Ssp][fl2r]U[Ssp][f]2r]U[Ssp][fl2r]C[Ssp][Sp9][Ssp][fl2r]U[Ssp][fl2r]A[Ssp][fl2r] | UUUCUACAUUU |
| | C[Ssp][f]2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | r]U[Ssp]mC[n001R]mC | |
| ADR- | mC[n001R]mU[Ssp]mC[Ssp][fl2r]U[Ssp][fl2r]G[Ssp][fl2r]U[Ssp][fl2r]A[Ssp][fl2r]G[Ssp][fl2r]U[Ssp][fl2r] | CUCUGUAGUCC |
| 0106840 | C[Ssp][fl2r]C[Ssp][fl2r]U[Ssp][fl2r]U[Ssp][fl2r]U[Ssp][fl2r]C[Ssp][Sp12][sp][fl2r]U[Ssp][fl2r]A[Ssp][fl2r] | UUUCUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | r]U[Ssp]mC[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0106841 | A[Ssp][fl2r]U[Ssp][fl2r]U[Ssp][fl2r]U[Ssp][fl2r]U[Ssp][fl2r]C[Ssp][SpC3][Ssp][fl2r]U[Ssp][fl2r]A[Ssp][f12 | UUUCUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | 2r]U[Ssp]mC[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0106842 | A[Ssp][fl2r]U[Ssp][fl2r]U[Ssp][fl2r]U[Ssp][fl2r]U[Ssp][fl2r]C[Ssp][oC40][Ssp][fl2r]U[Ssp][fl2r]A[Ssp][fl2 | UUUCUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0106843 | A[Ssp][fl2r]U[Ssp][fl2r]U[Ssp][fl2r]U[Ssp][fl2r]U[Ssp][fl2r]C[Ssp][oC60][Ssp][fl2r]U[Ssp][fl2r]A[Ssp][f12 | UUUCUACAUUU |
| | r]C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl | UT[3nU]ACUCC |
| | 2r]U[Ssp]mC[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0106844 | A[Ssp][fl2r]U[Ssp][fl2r]U[Ssp][fl2r]U[Ssp][fl2r]U[Ssp][fl2r]C[Ssp][Sp9][Ssp][fl2r]U[Ssp][fl2r]A[Ssp][fl2r] | UUUCUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | r]U[Ssp]mC[n001R]mC | |
| ADR- | mA[n001R]mA[Ssp]mU[Ssp][fl2r]C[Ssp][fl2r]U[Ssp][fl2r]U[Ssp][fl2r]U[Ssp][fl2r]C[Ssp][fl2r]C[Ssp][fl2r] | AAUCUUUCCAU |
| 0106845 | A[Ssp][fl2r]U[Ssp][fl2r]U[Ssp][fl2r]U[Ssp][fl2r]U[Ssp][fl2r]C[Ssp][Sp12][sp][fl2r]U[Ssp][fl2r]A[Ssp][fl2r] | UUUCUACAUUU |
| | C[Ssp][fl2r]A[Ssp][fl2r]U[Ssp][fl2r]U[Ssp][fl2r]U[Ssp][fl2r]U[Ssp]dT[Ssp]d[3nU][Ssp]dA[n001R]mCp[fl2 | UT[3nU]ACUCC |
| | r]U[Ssp]mC[n001R]mC | |
| ADR- | mG[n001R]mG[Ssp]mA[Ssp][fl2r]G[Ssp][fl2r]A[Ssp][fl2r]U[Ssp][fl2r]U[Ssp][f]2r]U[Ssp][fl2r]G[Ssp][fl2r] | GGAGAUUUGGG |
| 0106846 | G[Ssp][fl2r]G[Ssp][fl2r]C[Ssp][fl2r]U[Ssp][fl2r]U[Ssp][fl2r]C[Ssp][SpC3][Ssp][fl2r]U[Ssp][fl2r]U[Ssp][f12 | CUUCUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0106847 | G[Ssp][fl2r]G[Ssp][fl2r]C[Ssp][fl2r]U[Ssp][fl2r]U[Ssp][fl2r]C[Ssp][oC40][Ssp][fl2r]U[Ssp][fl2r]U[Ssp][fl2 | CUUCUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0106848 | G[Ssp][fl2r]G[Ssp][fl2r]C[Ssp][fl2r]U[Ssp][fl2r]U[Ssp][fl2r]C[Ssp][oC60][Ssp][fl2r]U[Ssp][fl2r]U[Ssp][fl2 | CUUCUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0106849 | G[Ssp][fl2r]G[Ssp][fl2r]C[Ssp][fl2r]U[Ssp][fl2r]U[Ssp][fl2r]C[Ssp][Sp9][Ssp][fl2r]U[Ssp][fl2r]U[Ssp][fl2r] | CUUCUUUCUGC |
| | U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2 | UC[3nU]CGCCGG |
| | r]C[Ssp]mC[Ssp]mG[n001R]mG | |
| ADR- | mG[n001R]mG[Ssp]mA[Ssp][fl2r]G[Ssp][fl2r]A[Ssp][fl2r]U[Ssp][fl2r]U[Ssp][fl2r]U[Ssp][fl2r]G[Ssp][fl2r] | GGAGAUUUGGG |
| 0106850 | G[Ssp][fl2r]G[Ssp][fl2r]C[Ssp][fl2r]U[Ssp][fl2r]U[Ssp][fl2r]C[Ssp][Sp12][sp][fl2r]U[Ssp][fl2r]U[Ssp][fl2r] | CUUCUUUCUGC |
| | G[Ssp][fl2r]G[Ssp][fl2r]C[Ssp][fl2r]U[Ssp][fl2r]U[Ssp][fl2r]C[Ssp][Sp12][sp][fl2r]U[Ssp][fl2r]U[Ssp][fl2r] | UC[3nU]CGCCGG |
| | r]C[Ssp]mC[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0106851 | G[Ssp][fl2r]A[Ssp][fl2r]G[Ssp][fl2r]A[Ssp][fl2r]U[Ssp][fl2r]U[Ssp][SpC3][Ssp][fl2r]U[Ssp][fl2r]U[Ssp][fl2 | GAUUUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0106852 | G[Ssp][fl2r]A[Ssp][fl2r]G[Ssp][fl2r]A[Ssp][fl2r]U[Ssp][fl2r]U[Ssp][oC40][Ssp][fl2r]U[Ssp][fl2r]U[Ssp][fl2 | GAUUUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0106853 | G[Ssp][fl2r]A[Ssp][fl2r]G[Ssp][fl2r]A[Ssp][fl2r]U[Ssp][fl2r]U[Ssp][oC60][Ssp][fl2r]U[Ssp][fl2r]U[Ssp][f12 | GAUUUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0106854 | G[Ssp][fl2r]A[Ssp][fl2r]G[Ssp][fl2r]A[Ssp][fl2r]U[Ssp][fl2r]U[Ssp][Sp9][Ssp][fl2r]U[Ssp][fl2r]U[Ssp][fl2r] | GAUUUUUCUGC |
| | U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2 | UC[3nU]CGCCGG |
| | r]C[Ssp]mC[Ssp]mG[n001R]mG | |
| ADR- | mG[n001R]mA[Ssp]mG[Ssp][fl2r]C[Ssp][fl2r]U[Ssp][fl2r]U[Ssp][fl2r]U[Ssp][fl2r]G[Ssp][fl2r]G[Ssp][fl2r] | GAGCUUUGGGA |
| 0106855 | G[Ssp][fl2r]A[Ssp][fl2r]G[Ssp][fl2r]A[Ssp][fl2r]U[Ssp][fl2r]U[Ssp][Sp12][sp][fl2r]U[Ssp][fl2r]U[Ssp][fl2r] | GAUUUUUCUGC |
| | U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2 | UC[3nU]CGCCGG |
| | r]C[Ssp]mC[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0106856 | C[Ssp][fl2r]U[Ssp][fl2r]G[Ssp][fl2r]C[Ssp][fl2r]C[Ssp][fl2r]A[Ssp][SpC3][Ssp][fl2r]U[Ssp][fl2r]U[Ssp][f12 | GCCAUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0106857 | C[Ssp][fl2r]U[Ssp][fl2r]G[Ssp][fl2r]C[Ssp][fl2r]C[Ssp][fl2r]A[Ssp][oC40][Ssp][fl2r]U[Ssp][fl2r]U[Ssp][fl2 | GCCAUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0106858 | C[Ssp][fl2r]U[Ssp][fl2r]G[Ssp][fl2r]C[Ssp][fl2r]C[Ssp][fl2r]A[Ssp][oC60][Ssp][fl2r]U[Ssp][fl2r]U[Ssp][fl2 | GCCAUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | 2r]C[Ssp]mC[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0106859 | C[Ssp][fl2r]U[Ssp][fl2r]G[Ssp][fl2r]C[Ssp][fl2r]C[Ssp][fl2r]A[Ssp][Sp9][Ssp][fl2r]U[Ssp][fl2r]U[Ssp][fl2r] | GCCAUUUCUGC |
| | r]U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl | UC[3nU]CGCCGG |
| | r]C[Ssp]mC[Ssp]mG[n001R]mG | |
| ADR- | mC[n001R]mC[Ssp]mC[Ssp][fl2r]C[Ssp][fl2r]G[Ssp][fl2r]G[Ssp][fl2r]C[Ssp][fl2r]C[Ssp][fl2r]U[Ssp][fl2r] | CCCCGGCCUCU |
| 0106860 | C[Ssp][fl2r]U[Ssp][fl2r]G[Ssp][fl2r]C[Ssp][fl2r]C[Ssp][fl2r]A[Ssp][Sp12][sp][fl2r]U[Ssp][fl2r]U[Ssp][fl2r] | GCCAUUUCUGC |
| | U[Ssp][fl2r]C[Ssp][fl2r]U[Ssp][fl2r]G[Ssp][fl2r]C[Ssp][fl2r]U[Ssp]dC[Ssp]d[3nU][Ssp]dC[n001R]mGp[fl2 | UC[3nU]CGCCGG |
| | r]C[Ssp]mC[Ssp]mG[n001R]mG | |
| ADR- | []r]G[n001R][r]A[Ssp][lr]A[Ssp][fl2r]G[Ssp][fl2r]U[Ssp][fl2r]C[Ssp][fl2r]A[Ssp][fl2r]A[Ssp][fl2r]A[Ssp][f | GAAGUCAAAUA |
| 0107290 | 12r]U[Ssp][fl2r]A[Ssp][fl2r]C[Ssp][fl2r]U[Ssp][fl2r]U[Ssp][fl2r]C[Ssp][fl2r]U[Ssp][fl2r]C[Ssp][fl2r]U[Ssp] | CUUCUCUAUAU |
| | [fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp | CT[3nU]GCCUC |
| | ]mU[n001R]mC | |
| ADR- | mG[n001R]mA[Ssp]mA[Ssp][fl2r]G[Ssp][fl2r]U[Ssp][fl2r]C[Ssp][fl2r]A[Ssp][fl2r]A[Ssp][fl2r]A[Ssp][fl2r] | GAAGUCAAAUA |
| 0107291 | U[Ssp][fl2r]A[Ssp][fl2r]C[Ssp][fl2r]U[Ssp][fl2r]U[Ssp][fl2r]C[Ssp][fl2r]U[Ssp][fl2r]C[Ssp][f]2r]U[Ssp][fl2 | CUUCUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp][lr | CT[3nU]GCCT[m5 |
| | ]T[n001R][lr][m5C] | C] |
| ADR- | [lr]G[n001R][lr]A[Ssp][lr]A[Ssp][fl2r]G[Ssp][fl2r]U[Ssp][fl2r]C[Ssp][fl2r]A[Ssp][fl2r]A[Ssp][fl2r]A[Ssp][f | GAAGUCAAAUA |
| 0107292 | 12r]U[Ssp][fl2r]A[Ssp][fl2r]C[Ssp][fl2r]U[Ssp][fl2r]U[Ssp][fl2r]C[Ssp][fl2r]U[Ssp][fl2r]C[Ssp][fl2r]U[Ssp] | CUUCUCUAUAU |
| | [fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp | CT[3nU]GCCT[m5 |
| | ][lr]T[n001R][lr][m5C] | C] |
| ADR- | mC[n001R]mU[Ssp]mG[Ssp][fl2r]A[Ssp][fl2r]A[Ssp][fl2r]G[Ssp][fl2r]U[Ssp][f]2r]C[Ssp][fl2r]A[Ssp][fl2r] | CUGAAGUCAAA |
| 0107293 | A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2r]U[Ssp][f12 | UACUUCUCUAU |
| | r]C[Ssp][fl2r]U[Ssp][fl2r]A[Ssp][fl2r]U[Ssp][fl2r]A[Ssp][fl2r][Ssp][fl2r][Ssp]dT[Ssp][3][Ssp]dG[n0 | AUCT[3nU]GCCU |
| | 01R]mCp[fl2r]C[Ssp]mU[n001R]mC | C |
| ADR- | [lr][m5C][n001R][lr]T[Ssp][lr]G[Ssp][fl2r]A[Ssp][fl2r]A[Ssp][fl2r]G[Ssp][fl2r]U[Ssp][fl2r]C[Ssp][fl2r]A[S | [m5C]TGAAGUC |
| 0107294 | sp][fl2r]A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2r]U[ | AAAUACUUCUC |
| | Ssp][fl2r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ss | UAUAUCT[3nU] |
| | p]dG[n001R]mCp[fl2r]C[Ssp]mU[n001R]mC | GCCUC |
| ADR- | mC[n001R]mU[Ssp]mG[Ssp][fl2r]A[Ssp][fl2r]A[Ssp][fl2r]G[Ssp][fl2r]U[Ssp][fl2r]C[Ssp][fl2r]A[Ssp][fl2r] | CUGAAGUCAAA |
| 0107295 | A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2r]U[Ssp][f12 | UACUUCUCUAU |
| | r]C[Ssp][fl2r]U[Ssp][fl2r]A[Ssp][fl2r]U[Ssp][fl2r]A[Ssp][fl2r]U[Ssp][fl2r][Ssp]dT[Ssp]d[3nU][Ssp]dG[n0 | AUCT[3nU]GCCT |
| | 01R]mCp[fl2r]C[Ssp][lr]T[n001R][lr][m5C] | [m5C] |
| ADR- | [lr][m5C][n001R][lr]T[Ssp][lr]G[Ssp][fl2r]A[Ssp][fl2r]A[Ssp][fl2r]G[Ssp][fl2r]U[Ssp][fl2r]C[Ssp][fl2r]A[S | [m5C]TGAAGUC |
| 0107296 | sp][fl2r]A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2r]U[ | AAAUACUUCUC |
| | Ssp][fl2r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ss | UAUAUCT[3nU] |
| | p]dG[n001R]mCp[fl2r]C[Ssp][lr]T[n001R][r][m5C] | GCCT[m5C] |
| ADR- | mU[n001R]mG[Ssp]mA[Ssp][fl2r]A[Ssp][fl2r]G[Ssp][fl2r]U[Ssp][fl2r]C[Ssp][fl2r]A[Ssp][fl2r]A[Ssp][fl2r] | UGAAGUCAAAU |
| 0107297 | A[Ssp][fl2r]U[Ssp][fl2r]A[Ssp][f]2r]C[Ssp][fl2r]U[Ssp][fl2r]U[Ssp][fl2r]C[Ssp][fl2r]U[Ssp][fl2r]C[Ssp][f12 | ACUUCUCUAUA |
| | r]U[Ssp][fl2r]A[Ssp][fl2r]U[Ssp][fl2r]A[Ssp][fl2r]U[Ssp][fl2r]C[Ssp]dT[Sspd]d[3nu][Ssp]dG[n001R]mCp[fl | UCT[3nU]GCCUC |
| | 2r]C[Ssp]mU[Ssp]mC[n001R]mC | C |
| ADR- | mC[n001R]mU[Ssp]mG[Ssp][fl2r]A[Ssp][fl2r]A[Ssp][fl2r]G[Ssp][fl2r]U[Ssp][fl2r]C[Ssp][fl2r]A[Ssp][fl2r] | CUGAAGUCAAA |
| 0107298 | A[Ssp][fl2r]A[Ssp][fl2r]U[Ssp][f]2r]A[Ssp][fl2r]C[Ssp][fl2r]U[Ssp][fl2r]U[Ssp][fl2r]C[Ssp][fl2r]U[Ssp][fl2 | UACUUCUCUAU |
| | r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ssp]dG[n0 | AUCT[3nU]GCCU |
| | 01R]mCp[fl2r]C[Ssp][fl2r]U[Ssp]mC[Ssp]mC[n001R]mA | CCA |
| ADR- | mA[n001R]mC[Ssp]mU[Ssp][fl2r]G[Ssp][fl2r]A[Ssp][fl2r]A[Ssp][fl2r]G[Ssp][fl2r]U[Ssp][fl2r]C[Ssp][fl2r] | ACUGAAGUCAA |
| 0107299 | A[Ssp][fl2r]A[Ssp][fl2r]A[Ssp][fl2r]U[Ssp][fl2r]A[Ssp][fl2r]C[Ssp][fl2r]U[Ssp][fl2r]U[Ssp][fl2r]C[Ssp][fl2 | AUACUUCUCUA |
| | r]U[Ssp][fl2r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU | UAUCT[3nU]GCC |
| | ][Ssp]dG[n001R]mCp[fl2r]C[Ssp][fl2r]U[Ssp][fl2r]C[Ssp]mC[Ssp]mA[n001R]mA | UCCAA |
| ADR- | [lr]T[n001R][[r]T[Ssp][lr][m5C][Ssp][fl2r]C[Ssp][fl2r]G[Ssp][fl2r]U[Ssp][fl2r]C[Ssp][fl2r]G[Ssp][fl2r]C[Ss | TT[m5C]CGUCG |
| 0107300 | p][fl2r]U[Ssp][fl2r]G[Ssp][fl2r]A[Ssp][fl2r]C[Ssp][fl2r]U[Ssp][fl2r]G[Ssp][fl2r]U[Ssp][fl2r]C[Ssp][fl2r]U[ | CUGACUGUCUA |
| | Ssp][fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C | UAUCT[3nU]GCC |
| | [Ssp]mU[n001R]mC | UC |
| ADR- | mU[n001R]mU[Ssp]mC[Ssp][fl2r]C[Ssp][fl2r]G[Ssp][fl2r]U[Ssp][fl2r]C[Ssp][fl2r]G[Ssp][fl2r]C[Ssp][fl2r] | UUCCGUCGCUG |
| 0107301 | U[Ssp][fl2r]G[Ssp][fl2r]A[Ssp][fl2r]C[Ssp][fl2r]U[Ssp][fl2r]G[Ssp][fl2r]U[Ssp][fl2r]C[Ssp][fl2r]U[Ssp][fl2 | ACUGUCUAUAU |
| | r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C[Ssp][lr | CT[3nU]GCCT |
| | ]T[n001R][lr][m5C] | [m5C] |
| ADR- | [lr]T[n001R][r]T[Ssp][lr][m5C][Ssp][fl2r]C[Ssp][fl2r]G[Ssp][fl2r]U[Ssp][fl2r]C[Ssp][fl2r]G[Ssp][fl2r]C[Ss | TT[m5C]CGUCG |
| 0107302 | p][fl2r]U[Ssp][fl2r]G[Ssp][fl2r]A[Ssp][fl2r]C[Ssp][fl2r]U[Ssp][fl2r]G[Ssp][fl2r]U[Ssp][fl2r]C[Ssp][fl2r]U[ | CUGACUGUCUA |
| | Ssp][fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl2r]C | UAUCT[3nU]GCC |
| | [Ssp][lr]T[n001R][lr][m5C] | T[m5C] |
| ADR- | mC[n001R]mU[Ssp]mU[Ssp][fl2r]U[Ssp][fl2r]C[Ssp][fl2r]C[Ssp][fl2r]G[Ssp][fl2r]U[Ssp][fl2r]C[Ssp][fl2r] | CUUUCCGUCGC |
| 0107303 | G[Ssp][fl2r]C[Ssp][fl2r]U[Ssp][fl2r]G[Ssp][fl2r]A[Ssp][fl2r]C[Ssp][fl2r]U[Ssp][fl2r]G[Ssp][fl2r]U[Ssp][fl2 | UGACUGUCUAU |
| | r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ssp]dG[n0 | AUCT[3nU]GCCU |
| | 01R]mCp[fl2r]C[Ssp]mU[n001R]mC | C |
| ADR- | [lr][m5C][n001R][lr]T[Ssp][lr]T[Ssp][fl2r]U[Ssp][fl2r]C[Ssp][fl2r]C[Ssp][fl2r]G[Ssp][fl2r]U[Ssp][fl2r]C[Ss | [m5C]TTUCCGU |
| 0107304 | p][fl2r]G[Ssp][fl2r]C[Ssp][fl2r]U[Ssp][fl2r]G[Ssp][fl2r]A[Ssp][fl2r]C[Ssp][fl2r]U[Ssp][fl2r]G[Ssp][fl2r]U[ | CGCUGACUGUC |
| | Ssp][fl2r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ss | UAUAUCT[3nU] |
| | p]dG[n001R]mCp[fl2r]C[Ssp]mU[n001R]mC | GCCUC |
| ADR- | mC[n001R]mU[Ssp]mU[Ssp][fl2r]U[Ssp][fl2r]C[Ssp][fl2r]C[Ssp][fl2r]G[Ssp][fl2r]U[Ssp][fl2r]C[Ssp][fl2r] | CUUUCCGUCGC |
| 0107305 | G[Ssp][fl2r]C[Ssp][fl2r]U[Ssp][fl2r]G[Ssp][fl2r]A[Ssp][fl2r]C[Ssp][fl2r]U[Ssp][fl2r]G[Ssp][fl2r]U[Ssp][fl2 | UGACUGUCUAU |
| | r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ssp]dG[n0 | AUCT[3nU]GCCT |
| | 01R]mCp[fl2r]C[Ssp][lr]T[n001R][lr][m5C] | [m5C] |
| ADR- | [lr][m5C][n001R][r]T[Ssp][lr]T[Ssp][fl2r]U[Ssp][fl2r]C[Ssp][fl2r]C[Ssp][fl2r]G[Ssp][fl2r]U[Ssp][fl2r]C[Ss | [m5C]TTUCCGU |
| 0107306 | p][fl2r]G[Ssp][fl2r]C[Ssp][fl2r]U[Ssp][fl2r]G[Ssp][fl2r]A[Ssp][fl2r]C[Ssp][fl2r]U[Ssp][fl2r]G[Ssp][fl2r]U[ | CGCUGACUGUC |
| | Ssp][fl2r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ss | UAUAUCT[3nU] |
| | p]dG[n001R]mCp[fl2r]C[Ssp][lr]T[n001R][lr][m5C] | GCCT[m5C] |
| ADR- | mU[n001R]mU[Ssp]mU[Ssp][fl2r]C[Ssp][fl2r]C[Ssp][fl2r]G[Ssp][fl2r]U[Ssp][fl2r]C[Ssp][fl2r]G[Ssp][fl2r] | UUUCCGUCGCU |
| 0107307 | C[Ssp][fl2r]U[Ssp][fl2r]G[Ssp][fl2r]A[Ssp][fl2r]C[Ssp][fl2r]U[Ssp][fl2r]G[Ssp][fl2r]U[Ssp][fl2r]C[Ssp][fl2 | GACUGUCUAUA |
| | r]U[Ssp][fl2r]A[Ssp][fl2r]U[Ssp][fl2r]A[Ssp][fl2r]U[Ssp][fl2r]C[Ssp]dT[Ssp]d[3nU][Ssp]dG[n001R]mCp[fl | UCT[3nU]GCCUC |
| | 2r]C[Ssp]mU[Ssp]mC[n001R]mC | C |
| ADR- | mC[n001R]mU[Ssp]mU[Ssp][fl2r]U[Ssp][fl2r]C[Ssp][fl2r]C[Ssp][fl2r]G[Ssp][fl2r]U[Ssp][fl2r]C[Ssp][fl2r] | CUUUCCGUCGC |
| 0107308 | G[Ssp][fl2r]C[Ssp][fl2r]U[Ssp][f]2r]G[Ssp][fl2r]A[Ssp][fl2r]C[Ssp][fl2r]U[Ssp][fl2r]G[Ssp][fl2r]U[Ssp][f12 | UGACUGUCUAU |
| | r]C[Ssp][fl2r]U[Ssp][fl2r]A[Ssp][fl2r]U[Ssp][fl2r]A[Ssp][fl2r]U[Ssp][fl2r]C[Ssp]dT[Ssp]d[3nU][Ssp]dG[n0 | AUCT[3nU]GCCU |
| | 01R]mCp[fl2r]C[Ssp][fl2r]U[Ssp]mC[Ssp]mC[n001R]mA | CCA |
| ADR- | mU[n001R]mC[Ssp]mU[Ssp][fl2r]U[Ssp][fl2r]U[Ssp][fl2r]C[Ssp][fl2r]C[Ssp][fl2r]G[Ssp][fl2r]U[Ssp][fl2r] | UCUUUCCGUCG |
| 0107309 | C[Ssp][fl2r]G[Ssp][fl2r]C[Ssp][fl2r]U[Ssp][fl2r]G[Ssp][fl2r]A[Ssp][fl2r]C[Ssp][fl2r]U[Ssp][fl2r]G[Ssp][fl2 | CUGACUGUCUA |
| | r]U[Ssp][fl2r]C[Ssp][fl2r]U[Ssp][fl2r]A[Ssp][fl2r]U[Ssp][fl2r]A[Ssp][fl2r]U[Ssp][f]2r]C[Ssp]dT[Ssp]d[3nU | UAUCT[3nU]GCC |
| | ][Ssp]dG[n001R]mCp[fl2r]C[Ssp][fl2r]U[Ssp][fl2r]C[Ssp]mC[Ssp]mA[n001R]mA | UCCAA |

Notes:
Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, m5, eo, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. A natural DNA sugar may also be indicated with “d” as in dG, dA, dC, and dT, and a natural phosphate linkage may be indicated with “p” in Table 1.

### Moieties and Modifications:

m: 2′-OMe;
f or [fl2r]: 2′-F;
m5: methyl at 5-position of C (nucleobase is 5-methylcytosine);
[m5C]: a nucleoside whose base is 5-methylcytosine;
*, PS, or [sp]: Phosphorothioate. It can be an end group (if it is an end group, e.g., a 5′-end group, it is indicated in the Description and typically not in Stereochemistry/Linkage), or a linkage, e.g., a linkage between linker and an oligonucleotide chain, an internucleotidic linkage (a phosphorothioate internucleotidic linkage), etc.;
[Ssp]: Phosphorothioate in the Sp configuration;
[n001R]: n001

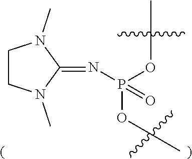

in Rp configuration;
b008U or [3nU]: a nucleoside whose base is

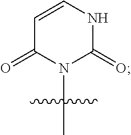 [SMILES: *n1c(=O)cc[nH]c1=O]

[lr]: a nucleoside whose sugar is a LNA sugar;
[SpC3]: a linker whose structure is —(CH₂)₃—;
[oC4o]: a linker whose structure is —(CH₂)₄—;
[oC6o]: a linker whose structure is -(CH₂)₆—;
[Sp9]: a linker whose structure is —CH₂CH₂OCH₂CH₂OCH₂CH₂—; and
[Sp12]: . a linker whose structure is —CH₂CH₂(OCH₂CH₂)₂OCH₂CH₂—.

### Oligonucleotide Compositions

Among other things, the present disclosure provides various oligonucleotide compositions. In some embodiments, the present disclosure provides oligonucleotide compositions of oligonucleotides described herein. In some embodiments, an oligonucleotide composition comprises a plurality of oligonucleotides described in the present disclosure. In some embodiments, an oligonucleotide composition is chirally controlled. In some embodiments, an oligonucleotide composition is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2n stereoisomers (e.g., when n is 10, 2\hat{ }10=1,032; when n is 20, 2\hat{ }20=1,048,576). These stereoisomers have the same constitution, but differ with respect to the pattern of stereochemistry of their linkage phosphorus.

In some embodiments, stereorandom oligonucleotide compositions have sufficient properties and/or activities for certain purposes and/or applications. In some embodiments, stereorandom oligonucleotide compositions can be cheaper, easier and/or simpler to produce than chirally controlled oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

In some embodiments, the present disclosure encompasses technologies for designing and preparing chirally controlled oligonucleotide compositions. In some embodiments, the present disclosure provides chirally controlled oligonucleotide compositions, e.g., of many oligonucleotides in Table 1 which contain S and/or R in their stereochemistry/linkage. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure. In some embodiments, oligonucleotides of a plurality are structural identical.

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”).

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the composition is enriched, relative to a substantially racemic preparation of oligonucleotides sharing the common base sequence, for oligonucleotides of the plurality.

In some embodiments, an oligonucleotide composition is a chirally controlled oligonucleotide composition comprising a plurality of oligonucleotides, wherein the oligonucleotides share:
a common base sequence,
a common pattern of backbone linkages, and
the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3,4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:
a common base sequence,
a common patter of backbone linkages, and
a common pattern of backbone chiral centers, which pattern comprises at least one Sp,
wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:
a common base sequence,
a common patter of backbone linkages, and
a common pattern of backbone chiral centers, which pattern comprises at least one Rp,
wherein the composition is enriched, relative to a substantially racemic preparation of oligonucleotides sharing the common base sequence and pattern of backbone linkages, for oligonucleotides of the plurality.

In some embodiments, the present disclosure provides a chirally controlled oligonucleotide composition comprising a plurality of oligonucleotides, wherein the oligonucleotides share:
1) a common constitution, and
2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),
wherein the composition is enriched, relative to a substantially racemic preparation of oligonucleotides of the common constitution, for oligonucleotides of the plurality.

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, an oligonucleotide composition is a chirally controlled oligonucleotide composition comprising a plurality of oligonucleotides, wherein the oligonucleotides share:
a common base sequence,
a common pattern of backbone linkages, and
the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3,4,5, 6,7, 8,9, 10, 11, 12, 13, 14, 15, 16, 17, 18, 19, 20, or more) chiral internucleotidic linkages (chirally controlled internucleotidic linkages),
wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, the present disclosure provides a chirally controlled oligonucleotide composition comprising a plurality of oligonucleotides, wherein the oligonucleotides share:
1) a common constitution, and
2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),
wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the common base sequence is complementary to a base sequence of a portion of a nucleic acid which portion comprises a target adenosine.

In some embodiments, the present disclosure provides an oligonucleotide composition comprising one or more pluralities of oligonucleotides, wherein oligonucleotides of each plurality independently share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the common base sequence of each plurality is independently complementary to a base sequence of a portion of a nucleic acid which portion comprises a target adenosine.

In some embodiments, the present disclosure provides an composition comprising a plurality of oligonucleotides which are of a particular oligonucleotide type characterized by:
a) a common base sequence;
b) a common pattern of backbone linkages;
c) a common pattern of backbone chiral centers;
d) a common pattern of backbone phosphorus modifications;
which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and
wherein the common base sequence is complementary to a base sequence of a portion of a nucleic acid which portion comprises a target adenosine.

In some embodiments, as described herein a portion can be about or at least about 10-40, 15-40, 20-40, e.g., 10, 11, 12, 13, 14, 15, 16, 17, 18, 19, 20, 21, 22, 23, 24, 25, 26, 27, 28, 29 or 30 or more, nucleobases long. In some embodiments, a portion is about or at least about or no more than about 1%-50% of a nucleic acid. In some embodiments, a portion is the whole length of a nucleic acid. In some embodiments, a common base sequence is complementary to a base sequence of a portion of a nucleic acid as described herein. In some embodiments, it is fully complementary across its length except at a nucleobase opposite to a target adenosine. In some embodiments, it is fully complementary across its length. In some embodiments, a target adenosine is associated with a condition, disorder or disease. In some embodiments, a target adenosine is a G to A mutation associated with a condition, disorder or disease. In some embodiments, a target adenosine is edited to I by a provided oligonucleotide or composition. In some embodiments, as described herein editing increases expression, level and/or activity of a transcript or a product thereof (e.g., a mRNA, a protein, etc.). In some embodiments, as described herein editing reduces expression, level and/or activity of a transcript or a product thereof (e.g., a mRNA, a protein, etc.).

In some embodiments, oligonucleotide of a plurality share the same nucleobase modifications and/or sugar modifications. In some embodiments, oligonucleotide of a plurality share the same internucleotidic linkage modifications (wherein the internucleotidic linkages may be in various acid, base, and/or salt forms). In some embodiments, oligonucleotides of a plurality share the same nucleobase modifications, sugar modifications, and internucleotidic linkage modifications, if any. In some embodiments, oligonucleotides of a plurality are of the same form, e.g., an acid form, a base form, or a particularly salt form (e.g., a pharmaceutically acceptable salt form, e.g., salt form). In some embodiments, oligonucleotides in a composition may exist as one or more forms, e.g., acid forms, base forms, and/or one or more salt forms. In some embodiments, in an aqueous solution (e.g., when dissolved in a buffer like PBS), anions and cations may dissociate. In some embodiments, oligonucleotides of a plurality are of the same constitution. In some embodiments, oligonucleotides of a plurality are structurally identical. In some embodiments, the present disclosure provides a chirally controlled oligonucleotide composition comprising a plurality of oligonucleotides, wherein the oligonucleotides are of a common constitution, and share the same linkage phosphorus stereochemistry at one or more (e.g., 1-60, 1-50. 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more) chiral internucleotidic linkages (chirally controlled internucleotidic linkages), wherein the composition is enriched, relative to a substantially racemic preparation of oligonucleotides of the common constitution, for oligonucleotides of the plurality.

In some embodiments, at least one chiral internucleotidic linkage is chirally controlled. In some embodiments, at least 2 internucleotidic linkages are independently chirally controlled. In some embodiments, the number of chirally controlled internucleotidic linkages is at least 3. In some embodiments, it is at least 4. In some embodiments, it is at least 5. In some embodiments, it is at least 6. In some embodiments, it is at least 7. In some embodiments, it is at least 8. In some embodiments, it is at least 9. In some embodiments, it is at least 10. In some embodiments, it is at least 11. In some embodiments, it is at least 12. In some embodiments, it is at least 13. In some embodiments, it is at least 14. In some embodiments, it is at least 15. In some embodiments, it is at least 20. In some embodiments, it is at least 25. In some embodiments, it is at least 30.

In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all chiral internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all phosphorothioate internucleotidic linkages are chirally controlled. In some embodiments, a percentage is at least 50%. In some embodiments, a percentage is at least 60%. In some embodiments, a percentage is at least 70%. In some embodiments, a percentage is at least 80%. In some embodiments, a percentage is at least 90%. In some embodiments, a percentage is at least 90%. In some embodiments, each chiral internucleotidic linkage is chirally controlled. In some embodiments, each phosphorothioate internucleotidic linkage is chirally controlled.

In some embodiments, no more than 1-10, e.g., no more than 1, 2, 3, 4, 5, 6, 7, 8, 9, or 10, chiral internucleotidic linkages are not chirally controlled. In some embodiments, no more than 1 chiral internucleotidic linkages is not chirally controlled. In some embodiments, no more than 2 chiral internucleotidic linkages are not chirally controlled. In some embodiments, no more than 3 chiral internucleotidic linkages are not chirally controlled. In some embodiments, no more than 4 chiral internucleotidic linkages are not chirally controlled. In some embodiments, no more than 5 chiral internucleotidic linkages are not chirally controlled. In some embodiments, the number of non-chirally controlled internucleotidic linkages is 1. In some embodiments, it is 2. In some embodiments, it is 3. In some embodiments, it is 4. In some embodiments, it is 5.

In some embodiments, the present disclosure provides a composition comprising a plurality of oligonucleotides, wherein each oligonucleotide of the plurality is independently a particular oligonucleotide or a salt thereof. In some embodiments, the present disclosure provides a composition comprising a plurality of oligonucleotides, wherein each oligonucleotide of the plurality is independently a particular oligonucleotide or a pharmaceutically acceptable salt thereof. In some embodiments, such a composition is enriched relative to a substantially racemic preparation of a particular oligonucleotide. As appreciated by those skilled in the art, oligonucleotides of the plurality share a common sequence which is the base sequence of the particular oligonucleotide. In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the base sequence of a the particular oligonucleotide are oligonucleotide of the plurality. In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%0, 40%, 50%, 60%, 70%, 80%, 85%, 90%, 91%, 92%, 93%, 94%, 95%, 96%, 97%, 98%, or 99% of all oligonucleotides in the composition that share the constitution of the particular oligonucleotide or a salt thereof are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%. In some embodiments, a particular oligonucleotide is an oligonucleotide exemplified herein, e.g., an oligonucleotide of Table 1 or another table.

In some embodiments, an enrichment relative to a substantially racemic preparation is that at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%0, 40%, 50%, 60%, 70% 80%, 85%, 90%, 91%, 92%, 93%, 94%, 95%, 96%, 97%, 98%, or 99% of all oligonucleotides in the composition, or all oligonucleotides in the composition that share the common base sequence of a plurality, or all oligonucleotides in the composition that share the common constitution of a plurality, are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%.

In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the common base sequence of a plurality are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments. it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%.

Levels of oligonucleotides of a plurality in chirally controlled oligonucleotide compositions are controlled. In contrast, in non-chirally controlled (or stereorandom, racemic) oligonucleotide compositions (or preparations), levels of oligonucleotides are random and not controlled. In some embodiments, an enrichment relative to a substantially racemic preparation is a level described herein.

In some embodiments, a level as a percentage (e.g., a controlled level, a pre-determined level, an enrichment) is or is at least (DS)^{nc}, wherein DS (diastereopurity of an individual internucleotidic linkage) is 90%-100%, and nc is the number of chirally controlled internucleotidic linkages as described in the present disclosure (e.g., 5, 6, 7, 8, 9, 10, 11, 12, 13, 14, 15, 16, 17, 18, 19, 20 or more). In some embodiments, each chiral internucleotidic linkage is chirally controlled, and nc is the number of chiral internucleotidic linkage. In some embodiments, DS is 90%, 91%, 92%, 93%, 94%, 95%, 96%, 97%, 98%, 99% or 99.5% or more. In some embodiments, DS is or is at least 90%. In some embodiments, DS is or is at least 91%. In some embodiments, DS is or is at least 92%. In some embodiments, DS is or is at least 93%. In some embodiments, DS is or is at least 94%. In some embodiments, DS is or is at least 95%. In some embodiments, DS is or is at least 96%. In some embodiments, DS is or is at least 97%. In some embodiments, DS is or is at least 98%. In some embodiments, DS is or is at least 99%. In some embodiments, a level (e.g., a controlled level, a pre-determined level, an enrichment) is a percentage of all oligonucleotides in a composition that share the same constitution, wherein the percentage is or is at least (DS)^{nc}. For example, when DS is 99% and nc is 10, the percentage is or is at least 90% ((99%)¹⁰≈0.90=90%). As appreciated by those skilled in the art, in a stereorandom preparation the percentage is typically about 1/2^{nc}—when nc is 10, the percentage is about 1/2¹⁰≈0.001=0.1%. In some embodiments, an enrichment (e.g., relative to a substantially racemic preparation), a level, etc., is that at least about (DS)^{nc} of all oligonucleotides in the composition, or all oligonucleotides in the composition that share the common base sequence of a plurality, or all oligonucleotides in the composition that share the common constitution of a plurality, are oligonucleotide of the plurality. In some embodiments, it is of all oligonucleotides in the composition. In some embodiments, it is of all oligonucleotides in the composition that share the common base sequence of a plurality. In some embodiments, it is of all oligonucleotides in the composition that share the common constitution of a plurality. In some embodiments, various forms (e.g., various salt forms) of an oligonucleotide may be properly considered to have the same constitution.

In some embodiments, oligonucleotides comprise one or more (e.g., 5, 6, 7, 8, 9, 10, 11, 12, 13, 14, 15, 16, 17, 18, 19, 20 or more) chirally controlled chiral internucleotidic linkages the diastereomeric excess (d.e.) of whose linkage phosphorus is independently about or at least about 80%, 85%, 90%, 91%, 92%, 93%, 94%, 95%, 96%, 97%, 98% or 99%. In some embodiments, about or at least about 50%, 60%, 70%, 75%, 80%, 85%, 90%, or 95% of all chiral internucleotidic linkages comprising a chiral linkage phosphorus are independently such a chirally controlled internucleotidic linkage. In some embodiments, about or at least about 50%, 60%, 70%, 75%, 80%, 85%, 90%, or 95% of phosphorothioate internucleotidic linkages are independently such a chirally controlled internucleotidic linkage. In some embodiments, each phosphorothioate internucleotidic linkage is independently such a chirally controlled internucleotidic linkage. In some embodiments, each chiral internucleotidic linkage comprising a chiral linkage phosphorus is independently such a chirally controlled internucleotidic linkage. In some embodiments, d.e. is about or at least about 80%. In some embodiments, d.e. is about or at least about 85%. In some embodiments, d.e. is about or at least about 90%. In some embodiments, d.e. is about or at least about 95%. In some embodiments, d.e. is about or at least about 96%. In some embodiments, d.e. is about or at least about 97%. In some embodiments, d.e. is about or at least about 98%.

In some embodiments, an oligonucleotide composition (also referred to as an oligonucleotide composition) is a chirally controlled oligonucleotide composition comprising a plurality of oligonucleotides, wherein the oligonucleotides share:
a common base sequence,
a common pattern of backbone linkages, and
the same linkage phosphorus stereochemistry at one or more chiral internucleotidic linkages (chirally controlled internucleotidic linkages),
wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition (also referred to as an oligonucleotide composition) is a chirally controlled oligonucleotide composition comprising a plurality of oligonucleotides, wherein the oligonucleotides share:
a common base sequence,
a common patter of backbone linkages, and
a common pattern of backbone chiral centers, which pattern comprises at least one Sp,
wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, level of a diastereopurity of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy).

In some embodiments, a chirally controlled oligonucleotide composition comprises two or more pluralities of oligonucleotides, wherein each plurality is independently a plurality of oligonucleotides as described herein (e.g., in various chirally controlled oligonucleotide compositions). For example, in some embodiments, each plurality independently shares a common base sequence, and the same linkage phosphorus stereochemistry at one or more chiral internucleotidic linkages, and each plurality is independently enriched compared to stereorandom preparation of that plurality or each plurality is independently of a level as described herein. In some embodiments, at least two pluralities or each plurality independently targets a different adenosine. In some embodiments, at least two pluralities or each plurality independently targets a different transcript of the same or different nucleic acids. In some embodiments, at least two pluralities or each plurality independently targets transcripts of a different gene. Among other things. such compositions may be utilized to target two or more targets, in some embodiments, simultaneously and in the same system.

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure.

In some embodiments, a chirally controlled oligonucleotide composition is a chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein optionally combined with other structural features described herein, e.g., modifications of nucleobases, sugars, internucleotidic linkages, etc. can be utilized to provide to provide directed adenosine editing with high efficiency.

In some embodiments, an oligonucleotide composition comprises one or more internucleotidic linkages which are stereocontrolled (chirally controlled; in some embodiments, stereopure) and one or more internucleotidic linkages which are stereorandom. In some embodiments, an oligonucleotide composition comprises one or more internucleotidic linkages which are stereocontrolled (chirally controlled; in some embodiments, stereopure) and one or more internucleotidic linkages which are stereorandom.

In some embodiments, an oligonucleotide composition comprises one or more internucleotidic linkages which are stereocontrolled (e.g., chirally controlled or stereopure) and one or more internucleotidic linkages which are stereorandom. Such oligonucleotides may target various nucleic acids and may have various base sequences, and may provide efficient adenosine editing (e.g., conversion of A to I).

In some embodiments, the present disclosure provides a chirally controlled oligonucleotide composition. In some embodiments, provided chirally controlled oligonucleotide compositions comprise a plurality of oligonucleotides of the same constitution, and have one or more internucleotidic linkages. In some embodiments, a plurality of oligonucleotides, e.g., in a chirally controlled oligonucleotide composition, is a plurality of an oligonucleotide selected from Table 1 (and/or one or more of various salts forms thereof), wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of an oligonucleotide selected from Table 1 (and/or one or more of various salts forms thereof), wherein each phosphorothioate internucleotidic linkage in the oligonucleotide is independently chirally controlled (each phosphorothioate internucleotidic linkage is independently Rp or Sp). In some embodiments, an oligonucleotide composition, e.g., an oligonucleotide composition is a substantially pure preparation of a single oligonucleotide in that oligonucleotides in the composition that are not the single oligonucleotide are impurities from the preparation process of the single oligonucleotide, in some case, after certain purification procedures. In some embodiments, a single oligonucleotide is an oligonucleotide of Table 1, wherein each chiral internucleotidic linkage of the oligonucleotide is chirally controlled (e.g., indicated as S or R but not X in “Stereochemistry/Linkage”).

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled oligonucleotide composition which is capable of modulating level, activity or expression of a gene or a gene product thereof. In some embodiments, level, activity or expression of a gene or a gene product thereof is increased (e.g., through conversion of A to I to restore correct G to A mutations, to increase protein translation levels, to increase production of particular protein isoforms, to modulate splicing to increase levels of a particular splicing products and proteins encoded thereby, etc.), and in some embodiments, level, activity or expression of a gene or a gene product thereof is decreased (e.g., through conversion of A to I to create stop codon and/or alter codons, to decrease protein translation levels, to decrease production of particular protein isoforms, to modulate splicing to decrease levels of a particular splicing products and proteins encoded thereby, etc.), as compared to a reference condition (e.g., absence of oligonucleotides and/or compositions of the present disclosure, and/or presence of a reference oligonucleotide and/or oligonucleotide composition (e.g., oligonucleotides of the same base sequence but different modifications, stereorandom compositions of oligonucleotides of comparable structures (e.g., base sequence, modifications, etc.) but lack of stereochemical control, etc.).

In some embodiments, the present disclosure provides a chirally controlled oligonucleotide composition which is capable of increasing the level, activity or expression of a gene or a gene product thereof, and comprises a plurality of oligonucleotides which share a common base sequence described herein. In some embodiments, the present disclosure provides a chirally controlled oligonucleotide composition which is capable of decreasing the level, activity or expression of a gene or a gene product thereof, and comprises a plurality of oligonucleotides which share a common base sequence described herein.

In some embodiments, a provided chirally controlled oligonucleotide composition is a chirally controlled oligonucleotide composition comprising a plurality of oligonucleotide. In some embodiments, a chirally controlled oligonucleotide composition is a chirally pure (or “stereochemically pure”) oligonucleotide composition. In some embodiments, the present disclosure provides a chirally pure oligonucleotide composition of an oligonucleotide in Table 1, wherein each chiral internucleotidic linkage of the oligonucleotide is independently chirally controlled (Rp or Sp, e.g., can be determined from R or S but not X in “Stereochemistry/Linkage”). As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in an oligonucleotide ), and the chirally pure oligonucleotide composition does not contain any other stereoisomers (in the context of oligonucleotide, typically diastereomers as typically multiple chiral centers exist in an oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2” diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

Certain data showing properties and/or activities of chirally controlled oligonucleotide composition, e.g., chirally controlled oligonucleotide composition in modulating level, activity and/or expression of target genes and/or products thereof, are shown in, for example, the Examples of this disclosure.

In some embodiments, the present disclosure provides an oligonucleotide composition comprising oligonucleotides that comprise at least one chiral linkage phosphorus. In some embodiments, the present disclosure provides an oligonucleotide composition comprising oligonucleotides that comprise at least one chiral linkage phosphorus. In some embodiments, the present disclosure provides an oligonucleotide composition in which the oligonucleotides comprise a chirally controlled phosphorothioate internucleotidic linkage, wherein the linkage phosphorus has a Rp configuration. In some embodiments, the present disclosure provides an oligonucleotide composition in which the oligonucleotides comprise a chirally controlled phosphorothioate internucleotidic linkage, wherein the linkage phosphorus has a Sp configuration. In some embodiments, the present disclosure provides an oligonucleotide composition in which the oligonucleotides comprise a chirally controlled phosphorothioate internucleotidic linkage, wherein the linkage phosphorus has a Rp configuration and the linkage phosphorus has a Sp configuration. In some embodiments, such oligonucleotide compositions are chirally controlled, and the Rp and/or Sp internucleotidic linkages are independently chirally controlled internucleotidic linkages.

In some embodiments, compared to reference oligonucleotides or oligonucleotide compositions, provided oligonucleotides or oligonucleotide compositions (e.g., chirally controlled oligonucleotide compositions) are surprisingly effective. In some embodiments, desired biological effects (e.g., as measured by increased (if increase is desired) and/or decreased (if decrease is desired) levels of mRNA, proteins, etc. whose levels are targeted for increase) can be enhanced by more than 1.5, 2, 3, 4, 5, 6, 7, 8, 9, 10, 15, 20, 25, 30, 40, 50, or 100 fold (e.g., as measured by levels of desired mRNA, proteins, etc.). In some embodiments, a change is measured by increase of desired mRNA and/or protein levels, or decrease of undesired mRNA and/or protein levels, compared to a reference condition. In some embodiments, a change is measured by increase of a desired mRNA and/or protein level compared to a reference condition. In some embodiments, a change is measured by decrease of an undesired mRNA and/or level compared to a reference condition. In some embodiments, a reference condition is absence of provided oligonucleotides or oligonucleotide compositions, and or presence of reference oligonucleotides or oligonucleotide compositions, respectively. In some embodiments, a reference oligonucleotide shares the same base sequence, but different nucleobase modifications, sugar modifications, internucleotidic linkages modifications, and/or linkage phosphorus stereochemistry. In some embodiments, a reference oligonucleotide composition is a composition of oligonucleotides of the same base sequence, but different nucleobase modifications, sugar modifications, internucleotidic linkages modifications, and/or linkage phosphorus stereochemistry. In some embodiments, a reference composition for a chirally controlled oligonucleotide composition is a corresponding stereorandom composition of oligonucleotides having the same base sequence, nucleobase modifications, sugar modifications, and/or internucleotidic linkages modifications (but lack of and/or low levels of linkage phosphorus stereochemistry control), or having the same constitution.

In some embodiments, the present disclosure provides a chirally controlled oligonucleotide composition, wherein the linkage phosphorus of at least one chirally controlled internucleotidic linkage is Sp. In some embodiments, the present disclosure provides a chirally controlled oligonucleotide composition, wherein the majority of linkage phosphorus of chirally controlled internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled internucleotidic linkages (or of all chiral internucleotidic linkages, or of all internucleotidic linkages) are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled phosphorothioate internucleotidic linkages are Sp. In some embodiments, no more than 1, 2, 3, 4, 5, 6, 7, 8, 9, or 10 of phosphorothioate internucleotidic linkages are non-chirally controlled or are chirally controlled and Rp. In some embodiments, no more than 1, 2, 3, 4, 5, 6, 7, 8, 9, or 10 of phosphorothioate internucleotidic linkages are chirally controlled and Rp. In some embodiments, it is no more than 1. In some embodiments, it is no more than 2. In some embodiments, it is no more than 3. In some embodiments, it is no more than 4. In some embodiments, it is no more than 5. In some embodiments, each phosphorothioate internucleotidic linkage is independently chirally controlled. In some embodiments, the present disclosure provides a chirally controlled oligonucleotide composition, wherein the majority of chiral internucleotidic linkages are chirally controlled and are Sp at their linkage phosphorus. In some embodiments, the present disclosure provides a chirally controlled oligonucleotide composition, wherein each chiral internucleotidic linkage is chirally controlled and each chiral linkage phosphorus is Sp. In some embodiments, the present disclosure provides a chirally controlled oligonucleotide composition, e.g., chirally controlled oligonucleotide composition, wherein at least one chirally controlled internucleotidic linkage has a Rp linkage phosphorus. In some embodiments, the present disclosure provides a chirally controlled oligonucleotide composition, wherein at least one chirally controlled internucleotidic linkage comprises a Rp linkage phosphorus and at least one chirally controlled internucleotidic linkage comprises a Sp linkage phosphorus.

In some embodiments, the present disclosure provides a chirally controlled oligonucleotide composition, wherein at least two chirally controlled internucleotidic linkages have different linkage phosphorus stereochemistry and/or different P-modifications relative to one another, wherein a P-modification is a modification at a linkage phosphorus. In some embodiments, the present disclosure provides a chirally controlled oligonucleotide composition, wherein at least two chirally controlled internucleotidic linkages have different stereochemistry relative to one another, and the pattern of the backbone chiral centers of the oligonucleotides is characterized by a repeating pattern of alternating stereochemistry.

In certain embodiments, the present disclosure provides a chirally controlled oligonucleotide composition comprising a plurality of oligonucleotides, wherein with in each of the oligonucleotides at least two individual internucleotidic linkages have different P-modifications relative to one another. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage and a phosphorothioate triester internucleotidic linkage.

In some embodiments, the present disclosure provides a chirally controlled oligonucleotide composition, comprising a plurality of oligonucleotides which share a common base sequence that is the base sequence of an oligonucleotide disclosed herein, wherein at least one internucleotidic linkage is chirally controlled. In some embodiments, oligonucleotides are provided, administered or delivered as stereorandom compositions. In some embodiments, a stereorandom composition comprises a mixture of diastereomers, e.g., with diastereomers with respect to chiral linkage phosphorus centers.

### Linkage Phosphorus Stereochemistry and Pattern of Backbone Chiral Centers

Among other things, the present disclosure provides various oligonucleotide compositions. In some embodiments, the present disclosure provides oligonucleotide compositions of oligonucleotides described herein. In some embodiments, an oligonucleotide composition comprises a plurality of oligonucleotides described in the present disclosure. In some embodiments, an oligonucleotide composition is chirally controlled. In some embodiments, an oligonucleotide composition is not chirally controlled (stereorandom).

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, control of stereochemistry can provide improved properties and/or activities, including desired stability, reduced toxicity, improved modification of target nucleic acids, improved modulation of levels of transcripts and/or products (e.g., mRNA, proteins, etc.) encoded thereof, etc. In some embodiments, the present disclosure provides useful patterns of backbone chiral centers for oligonucleotides and/or regions thereof, which pattern includes a combination of stereochemistry of each chiral linkage phosphorus (Rp or Sp) of chiral linkage phosphorus, indication of each achiral linkage phosphorus (Op, if any), etc. from 5′ to 3′. Certain patterns are provided in various Tables (e.g., Stereochemistry/Linkage as examples; such patterns can be applied to various oligonucleotides with various base sequences and modifications (e.g., those described herein including patterns thereof).

Useful patterns of backbone chiral centers, e.g., those for oligonucleotides, first domains, second domains, first subdomains, second subdomains, third subdomains, etc., are extensively described herein. For example, in some embodiments, high levels of Sp internucleotidic linkages of oligonucleotides or of one or more portions thereof (e.g., first domains, second domains, first subdomains, second subdomains, and/or third subdomains, and/or 5′-end portions and/or 3′-end portions therein) provide high stability and/or activities. In some embodiments, first domains contain high levels of Sp internucleotidic linkages. In some embodiments, second domains contain high levels of Sp internucleotidic linkages (in numbers and/or percentages, relative to natural phosphate linkages and/or Rp internucleotidic linkages). In some embodiments, first subdomains contain high levels of Sp internucleotidic linkages. In some embodiments, second subdomains contain high levels of Sp internucleotidic linkages. In some embodiments, third subdomains contain high levels of Sp internucleotidic linkages. In some embodiments, Rp internucleotidic linkages can be utilized in various locations and/or portions. For example, in some embodiments, first domains contain one or more or high levels of Rp internucleotidic linkages, and in some embodiments, second subdomains contain one or more or high levels of Rp internucleotidic linkages.

In some embodiments, a number of linkage phosphorus in chirally controlled internucleotidic linkages are Sp. In some embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In some embodiments, at least 10%, 20%, 25%, 30%, 35%, 40%, 45% 50%, 55%, 60%, 65%, 70%, 75%, 80%, 85%, 90% or 95% of all internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all phosphorothioate internucleotidic linkages have Sp linkage phosphorus. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 65%. In some embodiments, the percentage is at least 70%. In some embodiments, the percentage is at least 75%. In some embodiments, the percentage is at least 80%. In some embodiments, the percentage is at least 90%. In some embodiments, the percentage is at least 95%. In some embodiments, all chirally controlled internucleotidic linkages have Sp linkage phosphorus. In some embodiments, all chirally controlled phosphorothioate internucleotidic linkages have Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In some embodiments, at least 5 internucleotidic linkages are chirally controlled internucleotidic linkages having Sp linkage phosphorus. In some embodiments, at least 6 internucleotidic linkages are chirally controlled internucleotidic linkages having Sp linkage phosphorus. In some embodiments, at least 7 internucleotidic linkages are chirally controlled internucleotidic linkages having Sp linkage phosphorus. In some embodiments, at least 8 internucleotidic linkages are chirally controlled internucleotidic linkages having Sp linkage phosphorus. In some embodiments, at least 9 internucleotidic linkages are chirally controlled internucleotidic linkages having Sp linkage phosphorus. In some embodiments, at least 10 internucleotidic linkages are chirally controlled internucleotidic linkages having Sp linkage phosphorus. In some embodiments, at least 11 internucleotidic linkages are chirally controlled internucleotidic linkages having Sp linkage phosphorus. In some embodiments, at least 12 internucleotidic linkages are chirally controlled internucleotidic linkages having Sp linkage phosphorus. In some embodiments, at least 13 internucleotidic linkages are chirally controlled internucleotidic linkages having Sp linkage phosphorus. In some embodiments, at least 14 internucleotidic linkages are chirally controlled internucleotidic linkages having Sp linkage phosphorus. In some embodiments, at least 15 internucleotidic linkages are chirally controlled internucleotidic linkages having Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 internucleotidic linkages are chirally controlled internucleotidic linkages having Rp linkage phosphorus. In some embodiments, one and no more than one internucleotidic linkage in an oligonucleotide is a chirally controlled internucleotidic linkage having Rp linkage phosphorus. In some embodiments, 2 and no more than 2 internucleotidic linkages in an oligonucleotide are chirally controlled internucleotidic linkages having Rp linkage phosphorus. In some embodiments, 3 and no more than 3 internucleotidic linkages in an oligonucleotide are chirally controlled internucleotidic linkages having Rp linkage phosphorus. In some embodiments, 4 and no more than 4 internucleotidic linkages in an oligonucleotide are chirally controlled internucleotidic linkages having Rp linkage phosphorus. In some embodiments. 5 and no more than 5 internucleotidic linkages in an oligonucleotide are chirally controlled internucleotidic linkages having Rp linkage phosphorus.

In some embodiments, all, essentially all or most of the internucleotidic linkages in an oligonucleotide or a portion thereof are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages in an oligonucleotide) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages in an oligonucleotide) being in the Rp configuration. In some embodiments, all, essentially all or most of the internucleotidic linkages in a first domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a first domain). In some embodiments, each internucleotidic linkage in a first domain is a phosphorothioate in the Sp configuration. In some embodiments, each internucleotidic linkage in the a domain is a phosphorothioate in the Sp configuration. In some embodiments, all, essentially all or most of the internucleotidic linkages in a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55% 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a second domain). In some embodiments, each internucleotidic linkage in a second domain is a phosphorothioate in the Sp configuration. In some embodiments, each internucleotidic linkage in a second domain is a phosphorothioate in the Sp configuration except for one phosphorothioate in the Rp configuration. In some embodiments, all, essentially all or most of the internucleotidic linkages in a subdomain of a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a first subdomain of a second domain). In some embodiments, each internucleotidic linkage in a first subdomain of a second domain is a phosphorothioate in the Sp configuration. In some embodiments, each internucleotidic linkage in a first subdomain of second domain is a phosphorothioate in the Sp configuration except for one phosphorothioate in the Rp configuration. In some embodiments, all, essentially all or most of the internucleotidic linkages in a the second subdomain of a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a second subdomain of a second domain) except for one or a minority of internucleotidic linkages being in the Rp configuration. In some embodiments, each internucleotidic linkage in a second subdomain of a second domain is a phosphorothioate in the Sp configuration except for one phosphorothioate in the Rp configuration. In some embodiments, each internucleotidic linkage in a second subdomain of a second domain is a phosphorothioate in the Sp configuration except for one phosphorothioate in the Rp configuration. In some embodiments, all, essentially all or most of the internucleotidic linkages in a the third subdomain of the second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a third subdomain of a second domain. In some embodiments, each internucleotidic linkage in a third subdomain of a second domain is a phosphorothioate in the Sp configuration except for one phosphorothioate in the Rp configuration. In some embodiments, each internucleotidic linkage in a third subdomain of a second domain is a phosphorothioate in the Sp configuration except for one phosphorothioate in the Rp configuration.

In some embodiments, an oligonucleotide comprises one or more Rp internucleotidic linkages. In some embodiments, an oligonucleotide comprises one and no more than one Rp internucleotidic linkages. In some embodiments, an oligonucleotide comprises five or more Rp internucleotidic linkages. In some embodiments, about 5%-50% of all chirally controlled internucleotidic linkages in an oligonucleotide are Rp. In some embodiments, about 5%-40% of all chirally controlled internucleotidic linkages in an oligonucleotide are Rp. In some embodiments, certain portions (e.g., domains, subdomains, etc.) may contain relatively more (in numbers and/or percentages) Rp internucleotidic linkages, e.g., second subdomains.

In some embodiments, an oligonucleotide comprises one or more Rp phosphorothioate internucleotidic linkages at one or more positions, e.g., −1, −2, +1, +2, +7, +8, etc. In some embodiments, an internucleotidic linkage at position −1 is a Rp phosphorothioate internucleotidic linkage. In some embodiments, an internucleotidic linkage at position −2 is a Rp phosphorothioate internucleotidic linkage. In some embodiments, an internucleotidic linkage at position +1 is a Rp phosphorothioate internucleotidic linkage. In some embodiments, an internucleotidic linkage at position +2 is a Rp phosphorothioate internucleotidic linkage. In some embodiments, two or three internucleotidic linkages at positions −1, −2, +1, and +2 Rp phosphorothioate internucleotidic linkages. In some embodiments, the positions are −1 and −2. In some embodiments, the positions are +1 and +2. In some embodiments, the positions are −1 and +1. In some embodiments, the positions are −1, +1 and +2. In some embodiments, the positions are −1, −2 and +1. In some embodiments, one and only one internucleotidic linkage is Rp phosphorothioate internucleotidic linkage. In some embodiments, one and only one internucleotidic linkage is Rp phosphorothioate internucleotidic linkage and is at position +2, +1, 1 or 2. In some embodiments, a position is +1. In some embodiments, a position is +2. In some embodiments, a position is −1. In some embodiments, a position is −2. In some embodiments, utilization of Rp internucleotidic linkages may improve editing efficiency by ADAR1 (p110 and/or p150) and/or ADAR2. In some embodiments, improvements of editing by ADAR1 (p110 and/or p150) are more than those by ADAR2 (no or less improvements or less editing compared to absence of Rp).

In some embodiments, the present disclosure provides a chirally controlled oligonucleotide composition wherein the composition comprises a non-random or controlled level of a plurality of oligonucleotides, wherein oligonucleotides of the plurality share a common base sequence, and share the same configuration of linkage phosphorus independently at 1-60, 1-50, 1-40, 1-30, 1-25, 1-20, 1-15, 1-10, 5-50, 5-40, 5-30, 5-25, 5-20, 5-15, 5-10, 1,2,3, 4, 5,6, 7, 8, 9, 10, 11, 12, 13, 14, 15, 16, 17, 18, 19, 20, 21, 22, 23, 24, or 25 or more chiral internucleotidic linkages.

In some embodiments, provided oligonucleotides comprise 2-30 chirally controlled internucleotidic linkages. In some embodiments, provided oligonucleotide compositions comprise 5-30 chirally controlled internucleotidic linkages. In some embodiments, provided oligonucleotide compositions comprise 10-30 chirally controlled internucleotidic linkages. In some embodiments, provided oligonucleotide compositions comprise 1, 2, 3, 4, 5, 6, 7, 8, 9, 10, 11, 12, 13, 14, 15, 16, 17, 18, 19, 20, 21, 22, 23, 24, or 25 or more chirally controlled internucleotidic linkages.

In some embodiments, about 1-100% of all internucleotidic linkages are chirally controlled internucleotidic linkages. In some embodiments, about 1-100% of all chiral internucleotidic linkages are chirally controlled internucleotidic linkages. In some embodiments, a percentage is about 5%-100%. In some embodiments, a percentage is at least 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a percentage is about 5%10%, 15%, 20%, 25%, 30%, 35%, 40%, 45%, 50%, 55%, 60%, 65%, 70%, 75%, 80%, 85%, 90%, 95%, 965, 96%, 98%, or 99%.

In some embodiments, an internucleotidic linkage in the Sp configuration (having a Sp linkage phosphorus) is a phosphorothioate internucleotidic linkage. In some embodiments, an achiral internucleotidic linkage is a natural phosphate linkage. In some embodiments, an internucleotidic linkage in the Rp configuration (having a Rp linkage phosphorus) is a phosphorothioate internucleotidic linkage. In some embodiments, each internucleotidic linkage in the Sp configuration is a phosphorothioate internucleotidic linkage. In some embodiments, each achiral internucleotidic linkage is a natural phosphate linkage. In some embodiments, each internucleotidic linkage in the Rp configuration is a phosphorothioate internucleotidic linkage. In some embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In some embodiments, provided oligonucleotides in chirally controlled oligonucleotide compositions each comprise different types of internucleotidic linkages. In some embodiments, provided oligonucleotides comprise at least one natural phosphate linkage and at least one modified internucleotidic linkage. In some embodiments, provided oligonucleotides comprise at least one natural phosphate linkage and 1,2,3,4, 5,6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 modified internucleotidic linkages. In some embodiments, a modified internucleotidic linkage is a phosphorothioate internucleotidic linkage. In some embodiments, each modified internucleotidic linkage is a phosphorothioate internucleotidic linkage. In some embodiments, each modified internucleotidic linkage is independently a chiral internucleotidic linkage and is independently chirally controlled.

In some embodiments, oligonucleotides in a chirally controlled oligonucleotide composition each comprise at least two internucleotidic linkages that have different stereochemistry and/or different P-modifications relative to one another. In some embodiments, at least two internucleotidic linkages have different stereochemistry relative to one another. In some embodiments, oligonucleotides each comprise a pattern of backbone chiral centers comprising alternating linkage phosphorus stereochemistry.

In some embodiments, a phosphorothioate triester linkage comprises a chiral auxiliary, which, for example, is used to control the stereoselectivity of a reaction, e.g., a coupling reaction in an oligonucleotide synthesis cycle. In some embodiments, a phosphorothioate triester linkage does not comprise a chiral auxiliary. In some embodiments, a phosphorothioate triester linkage is intentionally maintained until and/or during the administration of the oligonucleotide composition to a subject.

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%, 70%, 80%, 85%, 90%, 91%, 92%, 93%, 94%, 95%, 96%, 97%, 98%, 99%, or 99.5%. In some embodiments, a chirally controlled internucleotidic linkage is typically formed with a stereoselectivity of at least 85%, 87%, 90%, 91%, 92%, 93%, 94%, 95%, 96%, 97%, 98%, 99.5% or virtually 100% (in some embodiments, at least 85%; in some embodiments, at least 87%; in some embodiments, at least 90%; in some embodiments, at least 95%; in some embodiments, at least 96%; in some embodiments, at least 97%; in some embodiments, at least 98%; in some embodiments, at least 99%). In some embodiments, a stereoselectivity is at least 85%. In some embodiments, a stereoselectivity is at least 87%. In some embodiments, a stereoselectivity is at least 90%. In some embodiments, each coupling step independently has a stereoselectivity of virtually 100%.

In some embodiments, stereopurity of a chiral center, e.g., a chiral linkage phosphorus, in a composition is at least 60%, 70%, 80%, 85%, 87%, 90%, 91%, 92%, 93%, 94%, 95%, 96%, 97%, 98%, 99%, or 99.5%. In some embodiments, a stereopurity is at least 80%. In some embodiments, a stereopurity is at least 85%. In some embodiments, a stereopurity is at least 87%. In some embodiments, a stereopurity is at least 90%. In some embodiments, a stereopurity is virtually 100%. In some embodiments, each chirally controlled internucleotidic linkage independently has a stereochemical purity (typically diastereomeric purity for oligonucleotides with multiple chiral centers) of at least 85%, 87%, 90%, 91%, 92%, 93%, 94%, 95%, 96%, 97%, 98%, 99.5% or virtually 100% (in some embodiments, at least 85%; in some embodiments, at least 87%; in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a chirally controlled internucleotidic linkage has a stereochemical purity of at least 90%. In some embodiments, a majority of chirally controlled internucleotidic linkages independently have a stereochemical purity of at least 90%. In some embodiments, each chirally controlled internucleotidic linkage independently has a stereochemical purity of at least 90%. In some embodiments, each phosphorothioate internucleotidic linkage is independently chirally controlled. In some embodiments, at least 60%, 65%, 70%, 75%, 80%, 85%, 90%, or 95%, or all chirally controlled internucleotidic linkages are Sp. In some embodiments, at least 60%, 65%, 70%, 75%, 80%, 85%, 90%, or 95%, or all chirally controlled phosphorothioate internucleotidic linkages are Sp. In some embodiments, at least 60%, 65%, 70%, 75%, 80%, 85%, 90%, or 95%, or all phosphorothioate internucleotidic linkages are chirally controlled and are Sp.

Stereoselectivity and stereopurity may be assessed by various technologies. In some embodiments, stereoselectivity and/or stereopurity is virtually 100% in that when a composition is analyzed by an analytical method (e.g., NMR, HPLC, etc.), virtually all detectable stereoisomers has the intended stereochemistry.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)].

In some embodiments, in stereorandom (or racemic) preparations (or stereorandom/non-chirally controlled oligonucleotide compositions), at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages of the oligonucleotides independently have a stereochemical purity (typically diastereomeric purity for oligonucleotides comprising multiple chiral centers) less than about 60%, 65%, 70%, 75%, 80%, or 85% with respect to chiral linkage phosphorus of the internucleotidic linkage(s). In some embodiments, a stereochemistry purity (stereopurity) is less than about 60%. In some embodiments, a stereochemistry purity (stereopurity) is less than about 65%. In some embodiments, a stereochemistry purity (stereopurity) is less than about 70%. In some embodiments, a stereochemistry purity (stereopurity) is less than about 75%. In some embodiments, a stereochemistry purity (stereopurity) is less than about 80%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., an oligonucleotide ) each independently have a diastereomeric purity as described herein. In some embodiments, a diastereomeric purity is at least 85%. In some embodiments, a diastereomeric purity is at least 86%. In some embodiments, a diastereomeric purity is at least 87%. In some embodiments, a diastereomeric purity is at least 88%. In some embodiments, a diastereomeric purity is at least 89%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease S1, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, oligonucleotides sharing a common base sequence, a common pattern of backbone linkages, and a common pattern of backbone chiral centers share a common pattern of backbone phosphorus modifications and a common pattern of base modifications. In some embodiments, oligonucleotide compositions sharing a common base sequence, a common pattern of backbone linkages, and a common pattern of backbone chiral centers share a common pattern of backbone phosphorus modifications and a common pattern of nucleoside modifications. In some embodiments, oligonucleotides share a common base sequence, a common pattern of backbone linkages, and a common pattern of backbone chiral centers have identical structures.

In some embodiments, the present disclosure provides an oligonucleotide composition comprising a plurality of oligonucleotides capable of directing deamination of a target adenosine in a target nucleic acid, wherein oligonucleotides of the plurality are of a particular oligonucleotide type, which composition is chirally controlled in that it is enriched, relative to a substantially racemic preparation of oligonucleotides having the same base sequence, for oligonucleotides of the particular oligonucleotide type.

In some embodiments, a plurality of oligonucleotides or oligonucleotides of a particular oligonucleotide type in a provided oligonucleotide composition are oligonucleotides. In some embodiments, the present disclosure provides a chirally controlled oligonucleotide composition comprising a plurality of oligonucleotides, wherein the oligonucleotides share:
a common base sequence;
a common pattern of backbone linkages; and
the same linkage phosphorus stereochemistry at one or more chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality.

In some embodiments, the present disclosure provides a chirally controlled oligonucleotide composition comprising a plurality of oligonucleotides, wherein the oligonucleotides share: a common base sequence;
a common pattern of backbone linkages; and
a common pattern of backbone chiral centers, which composition is a substantially pure preparation of
a single oligonucleotide in that at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of the oligonucleotides in the composition have the common base sequence, the common pattern of backbone linkages, and the common pattern of backbone chiral centers.

In some embodiments, an oligonucleotide composition type is further defined by: 4) additional chemical moiety, if any.

In some embodiments, the percentage is at least about 10%. In some embodiments, the percentage is at least about 20%. In some embodiments, the percentage is at least about 30%. In some embodiments, the percentage is at least about 40%. In some embodiments, the percentage is at least about 50%. In some embodiments, the percentage is at least about 60%. In some embodiments, the percentage is at least about 70%. In some embodiments, the percentage is at least about 75%. In some embodiments, the percentage is at least about 80%. In some embodiments, the percentage is at least about 85%. In some embodiments, the percentage is at least about 90%. In some embodiments, the percentage is at least about 91%. In some embodiments, the percentage is at least about 92%. In some embodiments, the percentage is at least about 93%. In some embodiments, the percentage is at least about 94%. In some embodiments, the percentage is at least about 95%. In some embodiments, the percentage is at least about 96%. In some embodiments, the percentage is at least about 97%. In some embodiments, the percentage is at least about 98%. In some embodiments, the percentage is at least about 99%. In some embodiments, the percentage is or is greater than (DS)^{nc}, wherein DS and nc are each independently as described in the present disclosure.

In some embodiments, a plurality of oligonucleotides share the same constitution. In some embodiments, a plurality of oligonucleotides are identical (the same stereoisomer). In some embodiments, a chirally controlled oligonucleotide composition is a stereopure oligonucleotide composition wherein oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In some embodiments, a provided composition is characterized in that when it is contacted with a target nucleic acid [e.g., a transcript (e.g., pre-mRNA, mature mRNA, other types of RNA, etc. that hybridizes with oligonucleotides of the composition)], levels of the target nucleic acid and/or a product encoded thereby is reduced compared to that observed under a reference condition. In some embodiments, levels of a nucleic acid and/or a product thereof, which nucleic acid is a product of an A to I edition of a target nucleic acid, is increased. In some embodiments, a reference condition is selected from the group consisting of absence of the composition, presence of a reference composition, and combinations thereof. In some embodiments, a reference condition is absence of the composition. In some embodiments, a reference condition is presence of a reference composition. In some embodiments, a reference composition is a composition whose oligonucleotides do not hybridize with the target nucleic acid. In some embodiments, a reference composition is a composition whose oligonucleotides do not comprise a sequence that is sufficiently complementary to the target nucleic acid. In some embodiments, a reference composition is a composition whose oligonucleotides share the same base sequence but do not share the same nucleobase, sugar and/or internucleotidic linkage modifications. In some embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In some embodiments, the present disclosure provides a chirally controlled oligonucleotide composition comprising a plurality of oligonucleotides capable of directing deamination of a target adenosine in a target nucleic acid, wherein the oligonucleotides share:
a common base sequence,
a common pattern of backbone linkages, and
the same linkage phosphorus stereochemistry at one or more chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality,
the oligonucleotide composition being characterized in that, when it is contacted with a target sequence, deamination of the target adenosine in the target nucleic acid is improved relative to that observed under a reference condition selected from the group consisting of absence of the composition, presence of a reference composition, and combinations thereof.

As appreciated by those skilled in the art, deamination of a target adenosine can be assessed using various technologies. In some embodiments, a technology is sequencing, wherein a deaminated adenosine is detected as G or I. In some embodiments, deamination is assessed by levels of a product (e.g., RNA, protein (e.g., encoded by a sequence wherein a target A is replaced with I but is otherwise identical to a target nucleic acid), etc.).

In some embodiments, an oligonucleotide composition is a substantially pure preparation of a single oligonucleotide stereoisomer in that oligonucleotides in the composition that are of the same constitution but are not of the stereoisomer are impurities from the preparation process of said oligonucleotide stereoisomer, in some case, after certain purification procedures.

In some embodiments, the present disclosure provides oligonucleotides and oligonucleotide compositions that are chirally controlled, and in some embodiments, stereopure. For instance, in some embodiments, a provided composition contains non-random or controlled levels of one or more individual oligonucleotide types. In some embodiments, oligonucleotides of the same oligonucleotide type are identical.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5 mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)]. In some embodiments, a nucleobase is or comprise an optionally substituted ring having at least one nitrogen atom. In some embodiments, a nucleobase comprise Ring BA as described herein, wherein at least one monocyclic ring of Ring BA comprise a nitrogen ring atom.

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5 mC.

In some embodiments, utilization of certain nucleobases at certain locations (e.g., in a nucleoside opposite to a target adenosine and/or its adjacent nucleoside(s)) can provide oligonucleotides with improved properties and/or activities (e.g., adenosine editing to I). In some embodiments, a useful nucleobase is or comprises Ring BA as described herein. In some embodiments, a nucleobase in a nucleoside is or comprises Ring BA which has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a nucleobase is optionally substituted or protected, or optionally substituted or protected tautomer of: Table BA-1. Certain useful nucleobases.

TABLE BA-1
Certain useful nucleobases.

| | |
|---|---|
| zdnp | 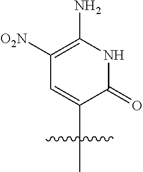 [SMILES: *c1cc([N+](=O)[O-])c(N)[nH]c1=O] |
| b007U | 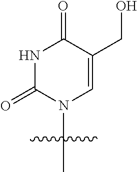 [SMILES: *n1cc(CO)c(=O)[nH]c1=O] |
| b001U | 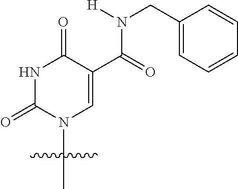 [SMILES: *n1cc(C(=O)NCc2ccccc2)c(=O)[nH]c1=O] |
| b001A or [o8A] | 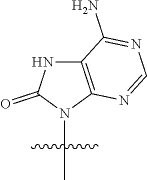 [SMILES: *n1c(=O)[nH]c2c(N)ncnc21] |
| b002U | 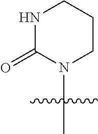 [SMILES: *N1CCCNC1=O] |
| b001C | 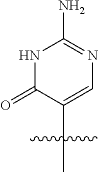 [SMILES: *c1cnc(N)[nH]c1=O] |
| b003U or [phc5U] | 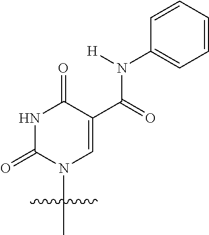 [SMILES: *n1cc(C(=O)Nc2ccccc2)c(=O)[nH]c1=O] |
| b002C | 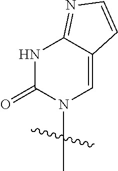 [SMILES: *n1cc2ccnc-2[nH]c1=O] |
| b004U or [nmc5U] | 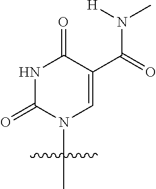 [SMILES: *n1cc(C(=O)NC)c(=O)[nH]c1=O] |
| b003C or [m6pyrC] | 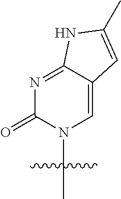 [SMILES: *n1cc2cc(C)[nH]c2nc1=O] |
| b005U or [dhpU] | 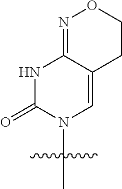 [SMILES: *N1C=C2CCON=C2NC1=O] |
| b002I or [Xan] | 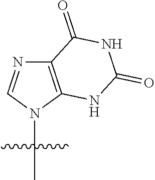 [SMILES: *n1cnc2c(=O)[nH]c(=O)[nH]c21] |
| b006U | 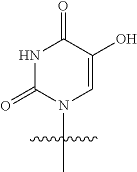 [SMILES: *n1cc(O)c(=O)[nH]c1=O] |
| b003I or [purine] | 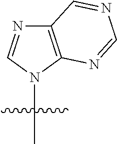 [SMILES: *n1cnc2cncnc21] |
| b008U or [3nU] | 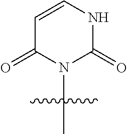 [SMILES: *n1c(=O)cc[nH]c1=O] |
| b009U or [im2o] | 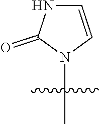 [SMILES: *n1cc[nH]c1=O] |
| b002A | 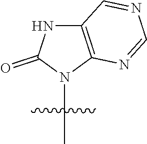 [SMILES: *n1c(=O)[nH]c2cncnc21] |
| b003A or [o2imbe] | 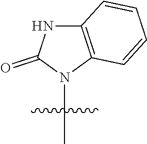 [SMILES: *n1c(=O)[nH]c2ccccc21] |
| b001G or [isoG] | 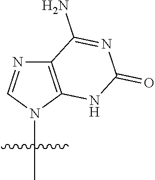 [SMILES: *n1cnc2c(N)nc(=O)[nH]c21] |
| b007C | 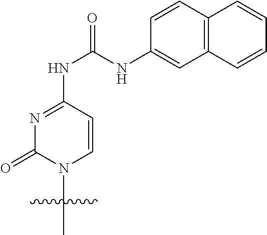 [SMILES: *n1ccc(NC(=O)Nc2ccc3ccccc3c2)nc1=O] |
| b004C or [pamC] | 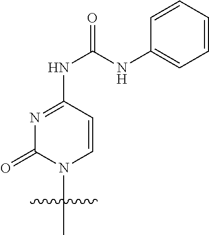 [SMILES: *n1ccc(NC(=O)Nc2ccccc2)nc1=O] |
| b005C or [no3pyr] | 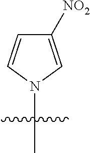 [SMILES: *n1ccc([N+](=O)[O-])c1] |
| b006C or [no5ind] | 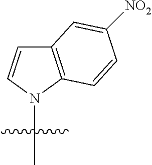 [SMILES: *n1ccc2cc([N+](=O)[O-])ccc21] |
| b008C or [mi5C] | 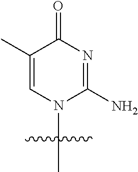 [SMILES: *n1cc(C)c(=O)nc1N] |
| b011U or [m3U] | 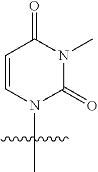 [SMILES: *n1ccc(=O)n(C)c1=O] |
| b012U or [o2pyr] | 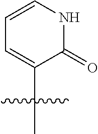 [SMILES: *c1ccc[nH]c1=O] |
| b013U or [3nm6U] | 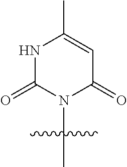 [SMILES: *n1c(=O)cc(C)[nH]c1=O] |
| b002G | 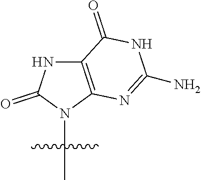 [SMILES: *n1c(=O)[nH]c2c(=O)[nH]c(N)nc21] |
| b004I or [o68pur] | 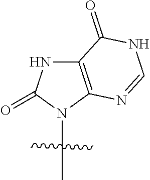 [SMILES: *n1c(=O)[nH]c2c(=O)[nH]cnc21] |
| b009C or [isoC] | 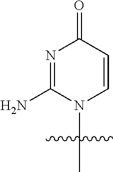 [SMILES: *n1ccc(=O)nc1N] |
| b014I | 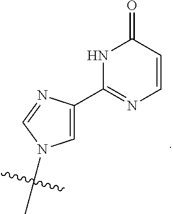 [SMILES: *n1cnc(-c2nccc(=O)[nH]2)c1] |
| b014U | 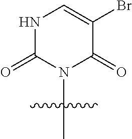 [SMILES: *n1c(=O)[nH]cc(Br)c1=O] |
| b015U | 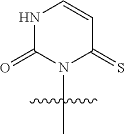 [SMILES: *n1c(=S)cc[nH]c1=O] |
| b004A or [n2A] | 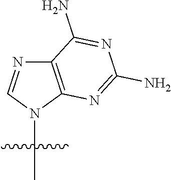 [SMILES: *n1cnc2c(N)nc(N)nc21] |
| b005A | 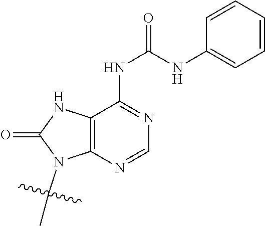 [SMILES: *n1c(=O)[nH]c2c(NC(=O)Nc3ccccc3)ncnc21] |
| b006A or [s8A] | 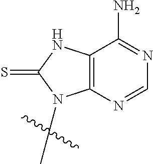 [SMILES: *n1c(=S)[nH]c2c(N)ncnc21] |
| b007A or [c7A] | 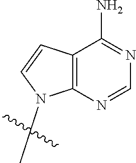 [SMILES: *n1ccc2c(N)ncnc21] |
| [3nT] | 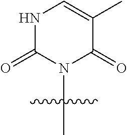 [SMILES: *n1c(=O)[nH]cc(C)c1=O] |
| [3ne5U] | 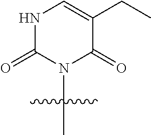 [SMILES: *n1c(=O)[nH]cc(CC)c1=O] |
| | 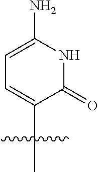 [SMILES: *c1ccc(N)[nH]c1=O] |
| | 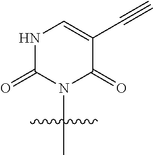 [SMILES: *n1c(=O)[nH]cc(C#C)c1=O] |
| [3nfl5U] | 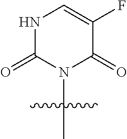 [SMILES: *n1c(=O)[nH]cc(F)c1=O] |
| [3npry5U] | 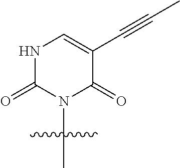 [SMILES: *n1c(=O)[nH]cc(C#CC)c1=O] |
| [3ncn5U] | 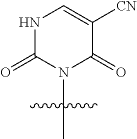 [SMILES: *n1c(=O)[nH]cc(C#N)c1=O] |
| | 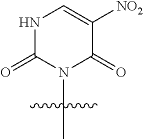 [SMILES: *n1c(=O)[nH]cc([N+](=O)[O-])c1=O] |
| | 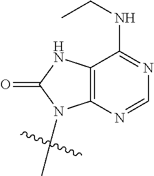 [SMILES: *n1c(=O)[nH]c2c(NCC)ncnc21] |
| | 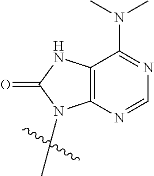 [SMILES: *n1c(=O)[nH]c2c(N(C)C)ncnc21] |
| | 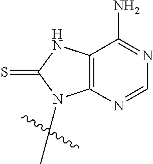 [SMILES: *n1c(=S)[nH]c2c(N)ncnc21] |
| | 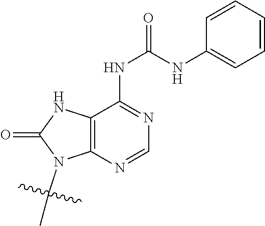 [SMILES: *n1c(=O)[nH]c2c(NC(=O)Nc3ccccc3)ncnc21] |
| [napth6o8A] | 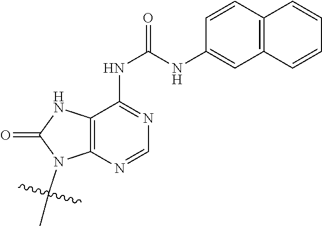 [SMILES: *n1c(=O)[nH]c2c(NC(=O)Nc3ccc4ccccc4c3)ncnc21] |
| | 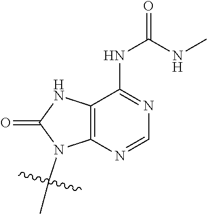 [SMILES: *n1c(=O)[nH]c2c(NC(=O)NC)ncnc21] |
| [ipr6o8A] | 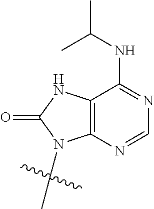 [SMILES: *n1c(=O)[nH]c2c(NC(C)C)ncnc21] |
| | 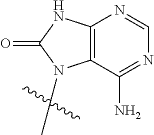 [SMILES: *n1c(=O)[nH]c2ncnc(N)c21] |
| | 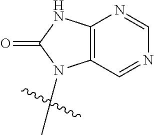 [SMILES: *n1c(=O)[nH]c2ncncc21] |
| | 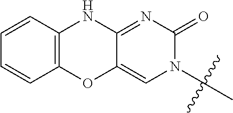 [SMILES: *n1cc2c(nc1=O)Nc1ccccc1O2] |
| | 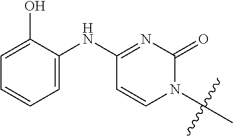 [SMILES: *n1ccc(Nc2ccccc2O)nc1=O] |
| [c7In] | 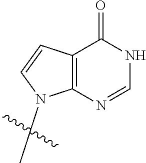 [SMILES: *n1ccc2c(=O)[nH]cnc21] |
| [c39z48In] | 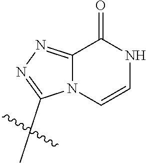 [SMILES: *c1nnc2c(=O)[nH]ccn12] |
| [z2c3In] | 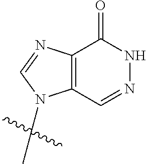 [SMILES: *n1cnc2c(=O)[nH]ncc21] |
| [z5C] | 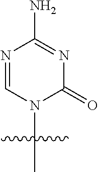 [SMILES: *n1cnc(N)nc1=O] |

In some embodiments, a modified nucleobase is b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b003I, b004I, b0141, [3nT], [3ne5U], [3nfl5U], [3npry5U], [3ncn5U], [nathp6o8A], [ipr6o8A], [c7Jn], [c39z481n], [z2c3In], [z5C], or zdnp. In some embodiments, the present disclosure provides oligonucleotides comprising one or more such nucleobases. In some embodiments, the present disclosure provides compounds comprising such nucleobases. In some embodiments, the present disclosure provides monomers (e.g., those useful for oligonucleotide synthesis) comprising such nucleobases. In some embodiments, the present disclosure provides phosphoramidites comprising such nucleobases. In some embodiments, phosphoramidites are CED phosphoramidites. In some embodiments, monomers comprise auxiliary moieties as described herein (e.g., with P forming bonds to O and N, to O and S, to S and S, etc.). In some embodiments, phosphoramidites comprise chiral auxiliary moieties as described herein (e.g., with P forming bonds to O and N). In some embodiments, R^{NS} comprises such a nucleobase. In some embodiments, nucleobases are protected for oligonucleotide synthesis.

In some embodiments, the present disclosure provides various nucleosides. In some embodiments, b001U, b002U, b003U, b004U, b005U, b006U, b008U, b009U, b011U, b012U, b013U, b014U, b015U, b002A, b001G, b004C, b007U, b001A, b002A, b003A, b004A, b005A, b006A, b007A, b001C, b002C, b003C, b002I, b003I, b009U, b003A, or b007C may also refer to a nucleoside whose nucleobase is b001U, b002U, b003U, b004U, b005U, b006U, b008U, b009U, b011U, b012U, b013U, b014U, b015U, b002A, b001G, b004C, b007U, b001A, b002A, b003A, b004A, b005A, b006A, b007A, b001C, b002C, b003C, b002I, b003I, b009U, b003A, or b007C, respectively. For example, b001A may refer to a nucleoside whose nucleobase is

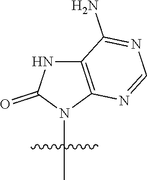 [SMILES: *n1c(=O)[nH]c2c(N)ncnc21]

and whose sugar is a natural DNA sugar; sugar modification may also be indicated, for example, “r” in b001rA indicates there is a 2′-OH on the sugar (a natural RNA sugar). In some embodiments, the present disclosure provides a compound having the structure of

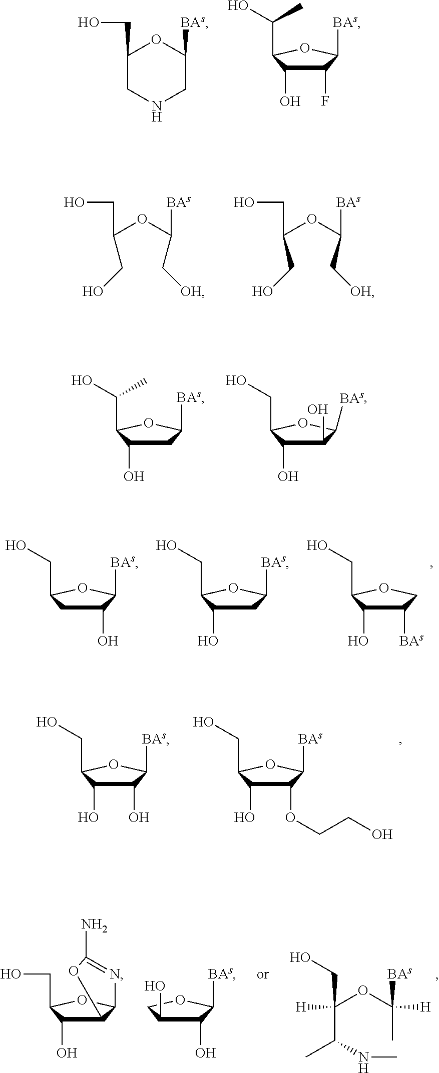

or a salt thereof, wherein BA^{s} is as described herein. In some embodiments, a provided compound, e.g., a nucleoside has the structure of

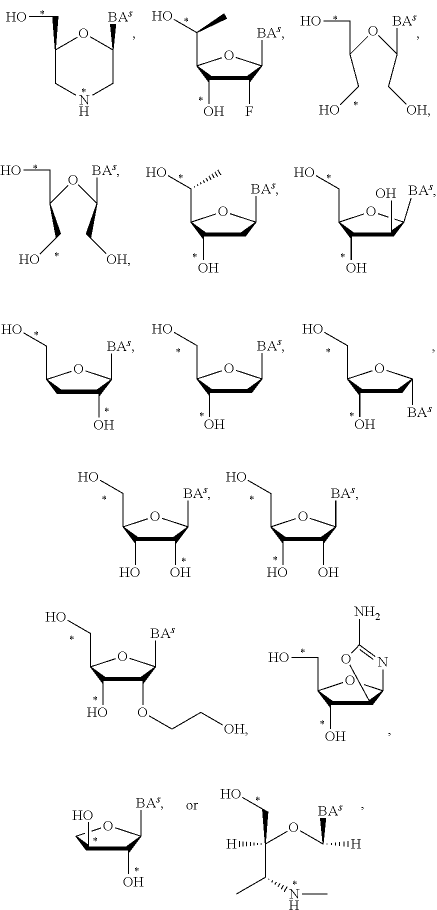

or a salt thereof, wherein “*” indicates connection to internucleotidic linkages when in various oligonucleotides, and BA^{s} is as described herein. In some embodiments, BA^{s} is a nucleobase, e.g., BA as described herein. In some embodiments, BA is protected for oligonucleotide synthesis. In some embodiments, a provided nucleoside is selected from Asm01

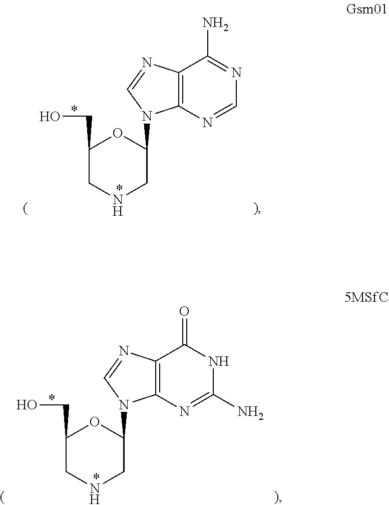 [SMILES: Nc1ncnc2c1ncn2[C@H]1CNC[C@@H](CO)O1 ; Nc1nc2c(ncn2[C@H]2CNC[C@@H](CO)O2)c(=O)[nH]1]

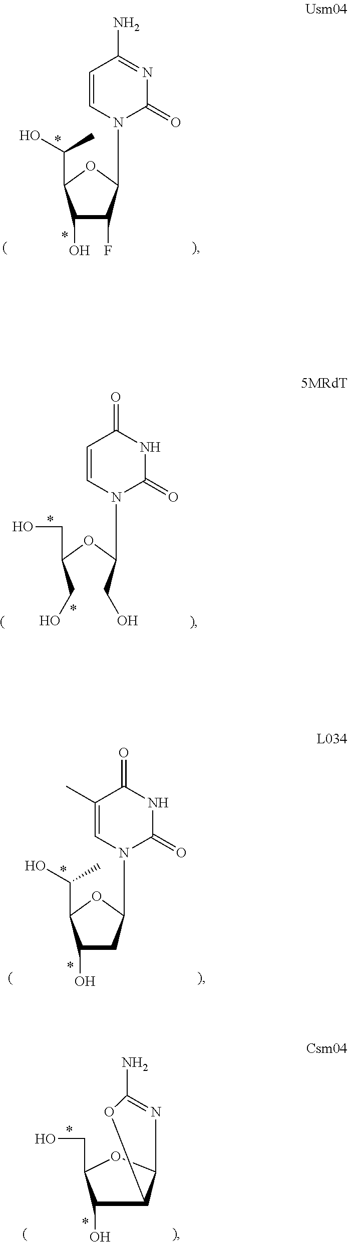

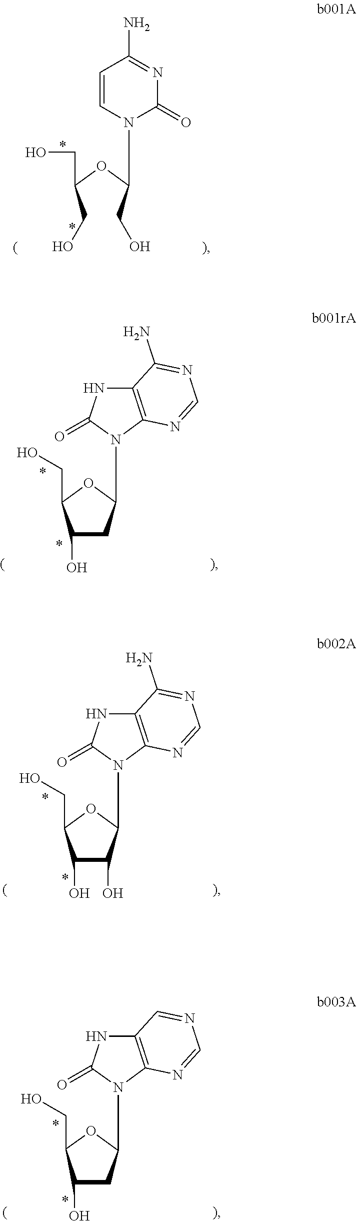

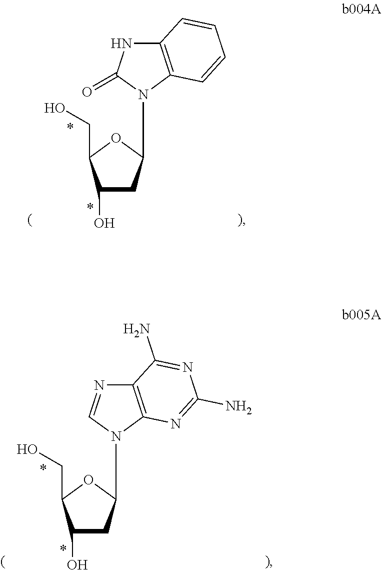 [SMILES: O=c1[nH]c2ccccc2n1[C@H]1C[C@@H](O)[C@@H](CO)O1 ; Nc1nc(N)c2ncn([C@H]3C[C@@H](O)[C@@H](CO)O3)c2n1]

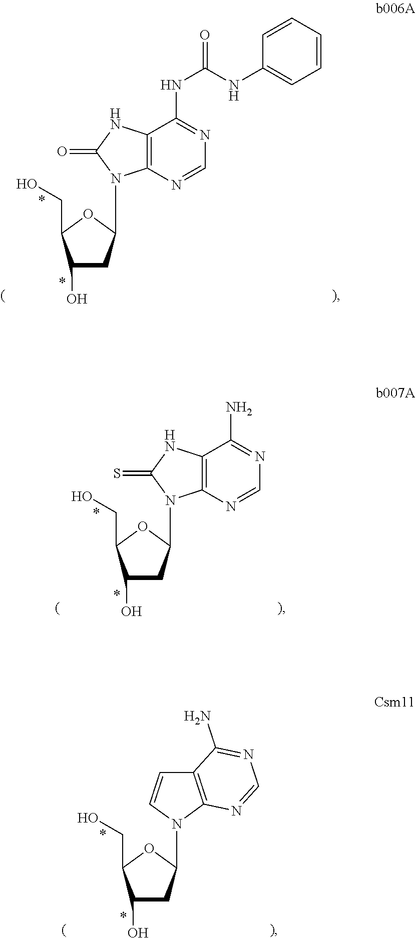 [SMILES: O=C(Nc1ccccc1)Nc1ncnc2c1[nH]c(=O)n2[C@H]1C[C@@H](O)[C@@H](CO)O1 ; Nc1ncnc2c1[nH]c(=S)n2[C@H]1C[C@@H](O)[C@@H](CO)O1 ; Nc1ncnc2c1ccn2[C@H]1C[C@@H](O)[C@@H](CO)O1]

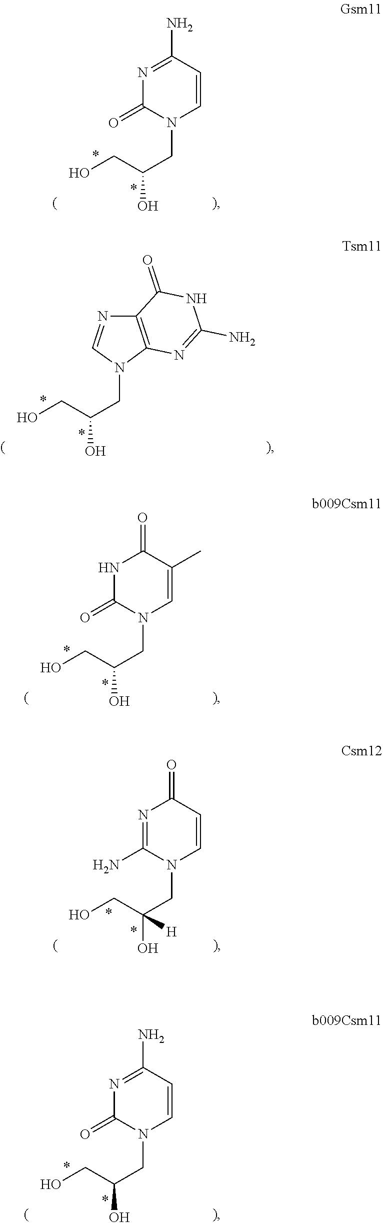 [SMILES: Nc1ccn(C[C@H](O)CO)c(=O)n1 ; Nc1nc2c(ncn2C[C@H](O)CO)c(=O)[nH]1 ; Cc1cn(C[C@H](O)CO)c(=O)[nH]c1=O ; Nc1nc(=O)ccn1C[C@H](O)CO ; Nc1ccn(C[C@@H](O)CO)c(=O)n1]

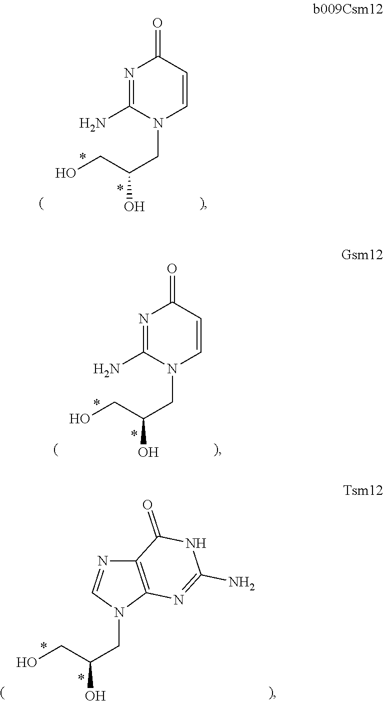 [SMILES: Nc1nc(=O)ccn1C[C@H](O)CO ; Nc1nc(=O)ccn1C[C@@H](O)CO ; Nc1nc2c(ncn2C[C@@H](O)CO)c(=O)[nH]1]

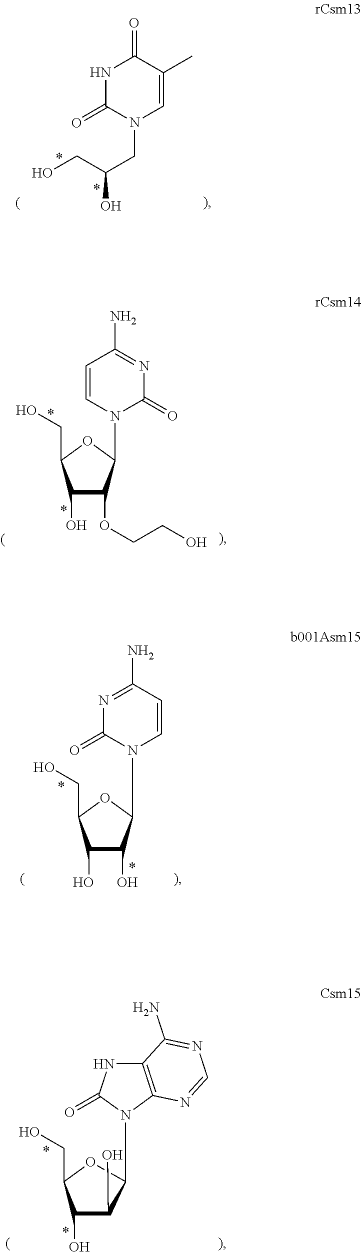

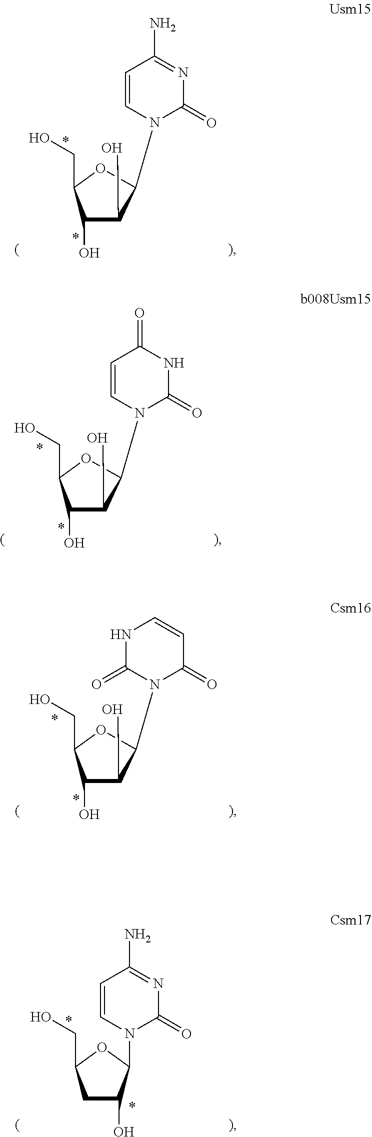

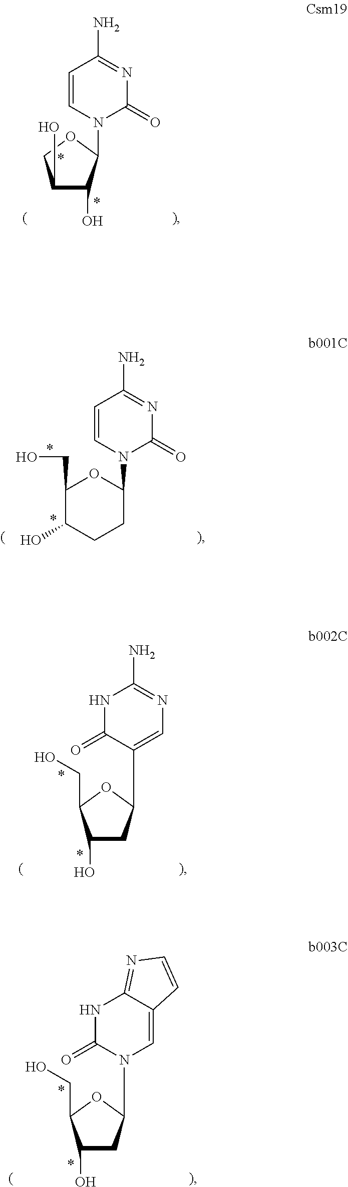

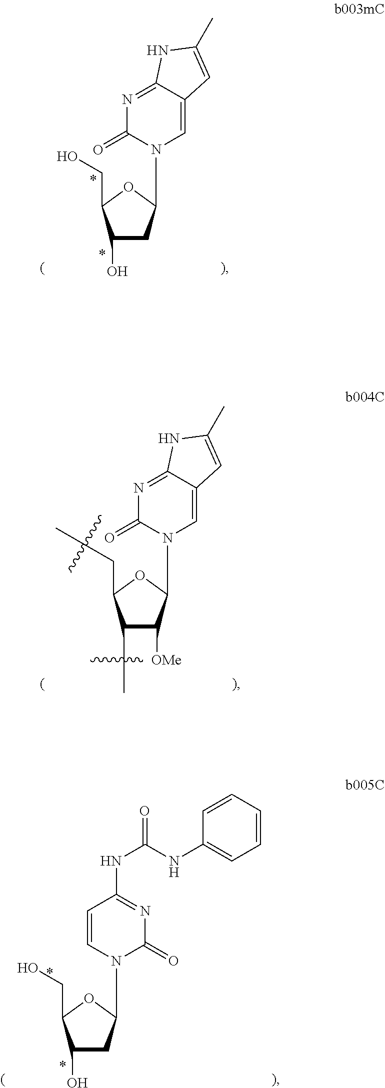

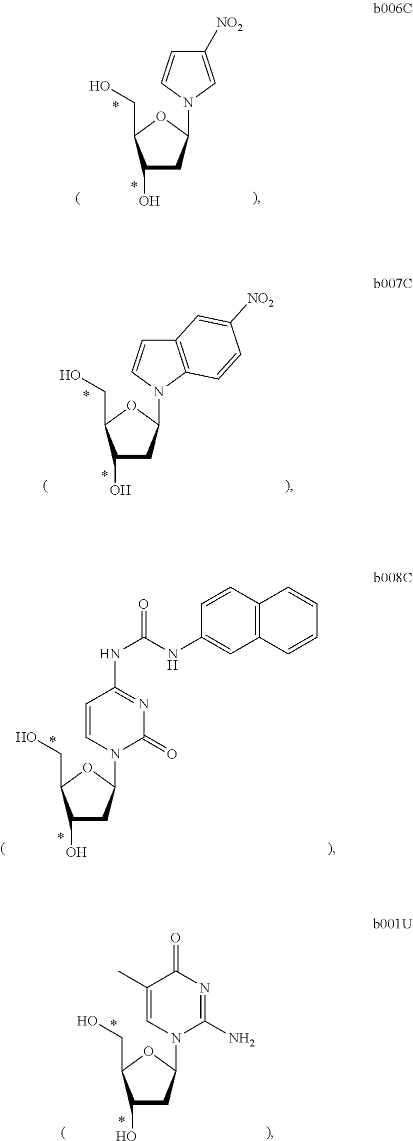 [SMILES: O=[N+]([O-])c1ccn([C@H]2C[C@@H](O)[C@@H](CO)O2)c1 ; O=[N+]([O-])c1ccc2c(ccn2[C@H]2C[C@@H](O)[C@@H](CO)O2)c1 ; O=C(Nc1ccc2ccccc2c1)Nc1ccn([C@H]2C[C@@H](O)[C@@H](CO)O2)c(=O)n1 ; Cc1cn([C@H]2C[C@@H](O)[C@@H](CO)O2)c(N)nc1=O]

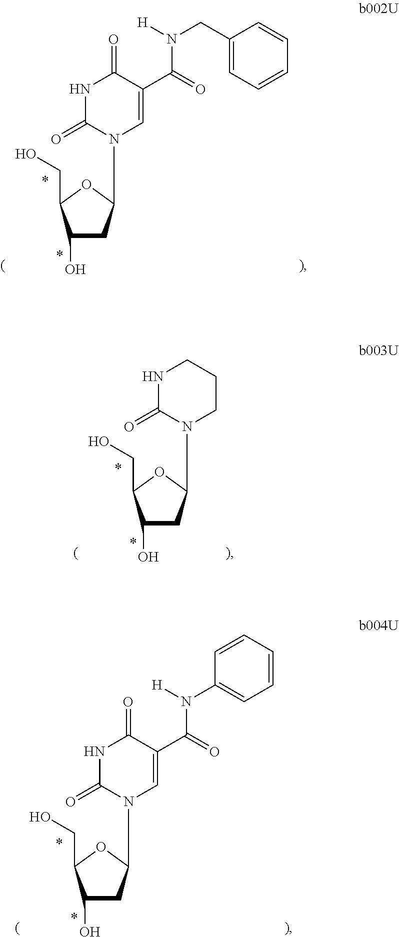 [SMILES: O=C(NCc1ccccc1)c1cn([C@H]2C[C@@H](O)[C@@H](CO)O2)c(=O)[nH]c1=O ; O=C1NCCCN1[C@H]1C[C@@H](O)[C@@H](CO)O1 ; O=C(Nc1ccccc1)c1cn([C@H]2C[C@@H](O)[C@@H](CO)O2)c(=O)[nH]c1=O]

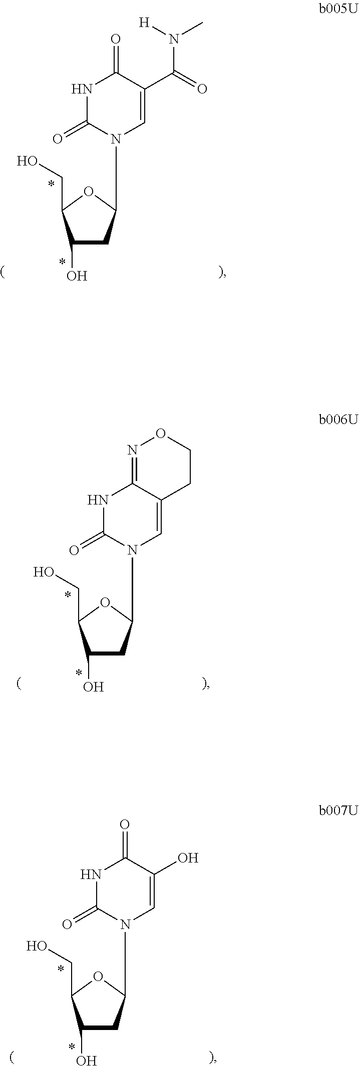 [SMILES: CNC(=O)c1cn([C@H]2C[C@@H](O)[C@@H](CO)O2)c(=O)[nH]c1=O ; O=C1NC2=NOCCC2=CN1[C@H]1C[C@@H](O)[C@@H](CO)O1 ; O=c1[nH]c(=O)n([C@H]2C[C@@H](O)[C@@H](CO)O2)cc1O]

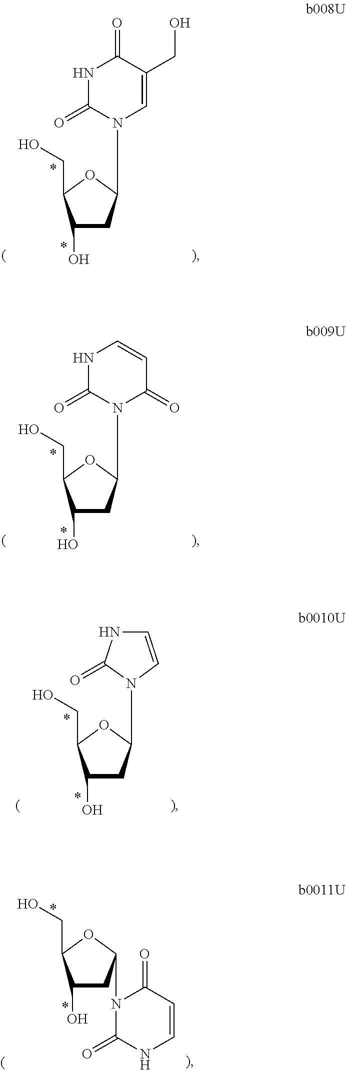 [SMILES: O=c1[nH]c(=O)n([C@H]2C[C@@H](O)[C@@H](CO)O2)cc1CO ; O=c1cc[nH]c(=O)n1[C@H]1C[C@@H](O)[C@@H](CO)O1 ; O=c1[nH]ccn1[C@H]1C[C@@H](O)[C@@H](CO)O1 ; O=c1cc[nH]c(=O)n1[C@@H]1C[C@@H](O)[C@@H](CO)O1]

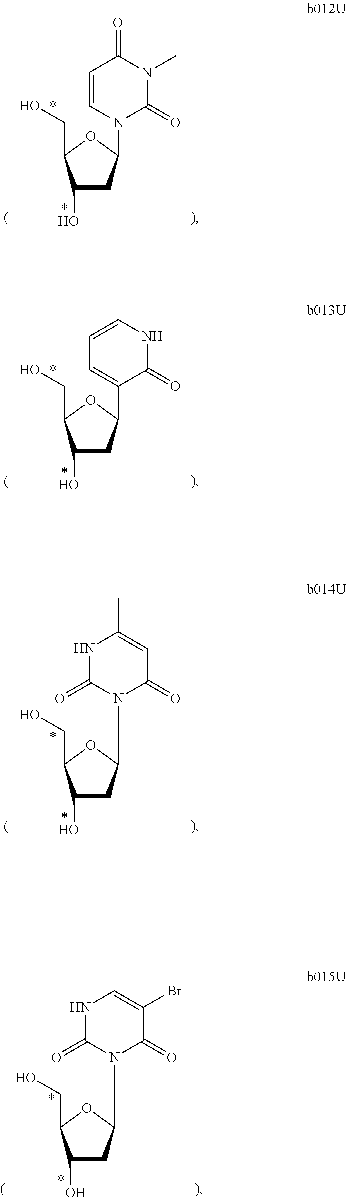 [SMILES: Cn1c(=O)ccn([C@H]2C[C@@H](O)[C@@H](CO)O2)c1=O ; O=c1[nH]cccc1[C@H]1C[C@@H](O)[C@@H](CO)O1 ; Cc1cc(=O)n([C@H]2C[C@@H](O)[C@@H](CO)O2)c(=O)[nH]1 ; O=c1[nH]cc(Br)c(=O)n1[C@H]1C[C@@H](O)[C@@H](CO)O1]

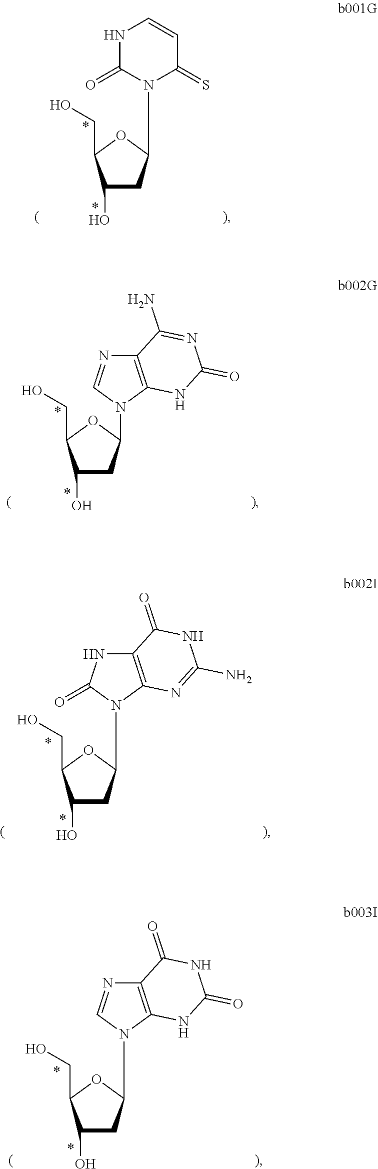 [SMILES: O=c1[nH]ccc(=S)n1[C@H]1C[C@@H](O)[C@@H](CO)O1 ; Nc1nc(=O)[nH]c2c1ncn2[C@H]1C[C@@H](O)[C@@H](CO)O1 ; Nc1nc2c([nH]c(=O)n2[C@H]2C[C@@H](O)[C@@H](CO)O2)c(=O)[nH]1 ; O=c1[nH]c(=O)c2ncn([C@H]3C[C@@H](O)[C@@H](CO)O3)c2[nH]1]

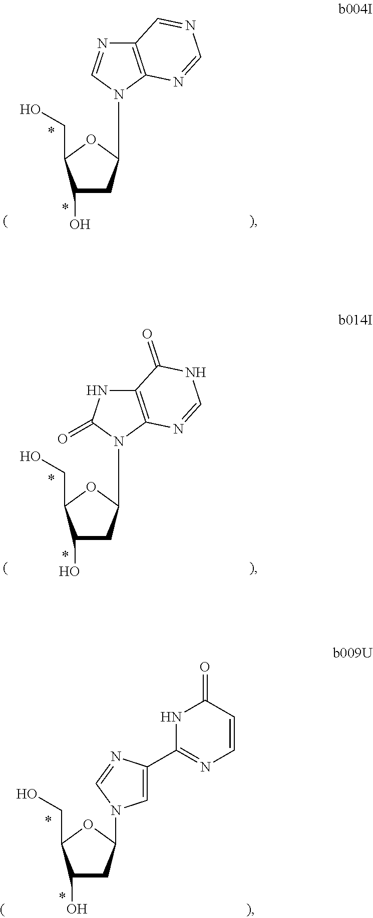 [SMILES: OC[C@H]1O[C@@H](n2cnc3cncnc32)C[C@H]1O ; O=c1[nH]cnc2c1[nH]c(=O)n2[C@H]1C[C@@H](O)[C@@H](CO)O1 ; O=c1ccnc(-c2cn([C@H]3C[C@@H](O)[C@@H](CO)O3)cn2)[nH]1]

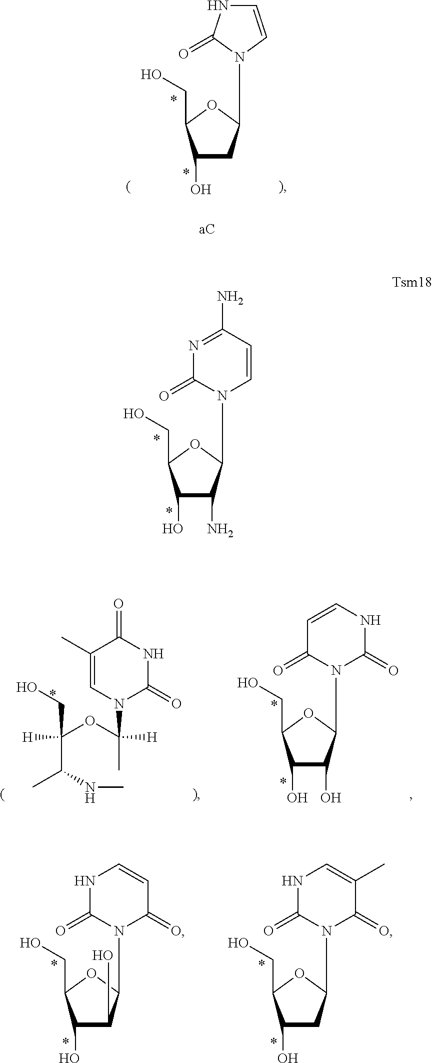

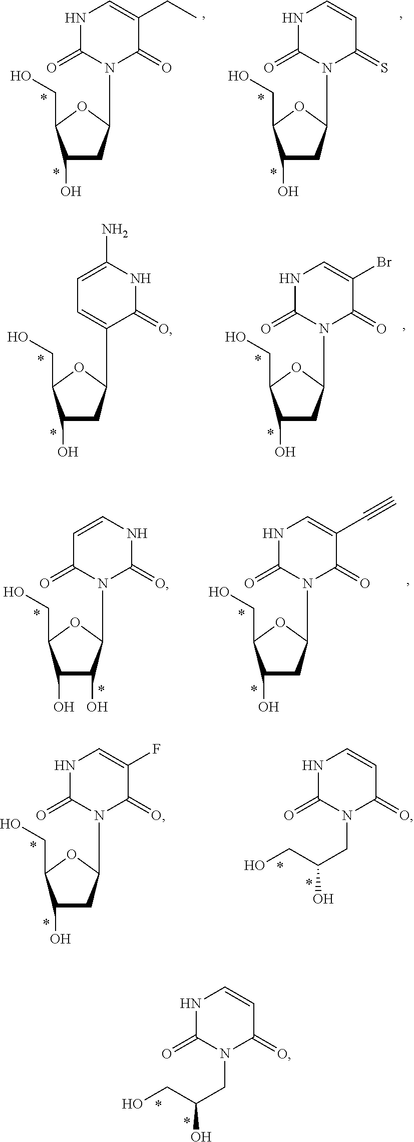

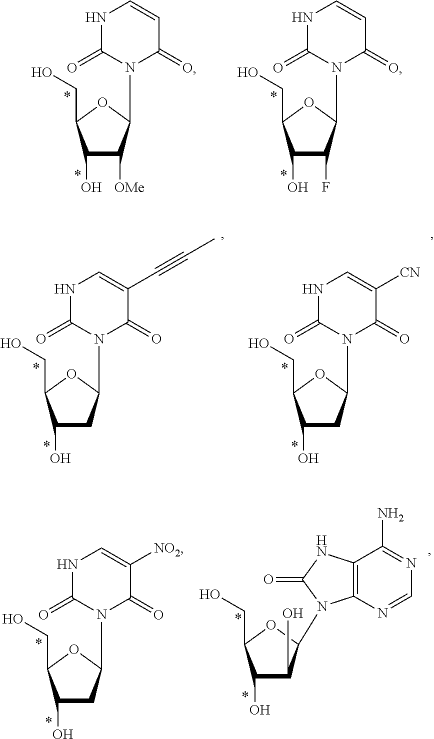

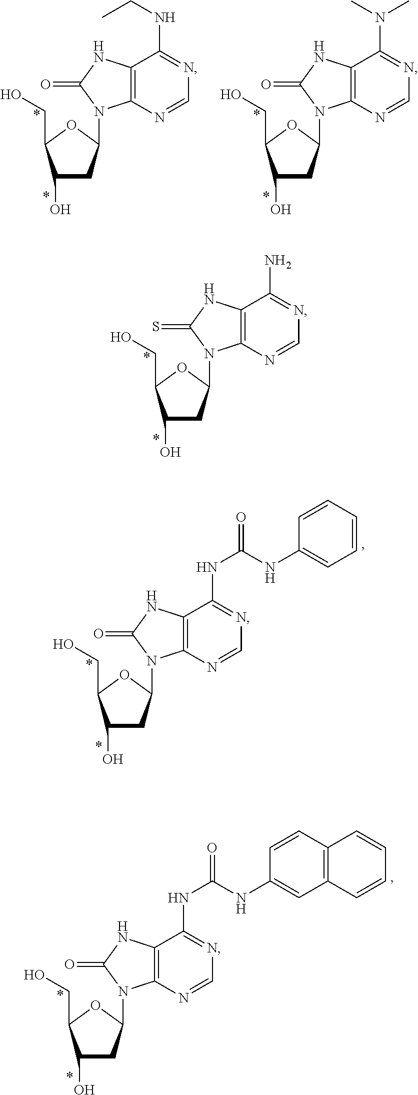 [SMILES: CCNc1ncnc2c1[nH]c(=O)n2[C@H]1C[C@@H](O)[C@@H](CO)O1 ; CN(C)c1ncnc2c1[nH]c(=O)n2[C@H]1C[C@@H](O)[C@@H](CO)O1 ; Nc1ncnc2c1[nH]c(=S)n2[C@H]1C[C@@H](O)[C@@H](CO)O1 ; O=C(Nc1ccccc1)Nc1ncnc2c1[nH]c(=O)n2[C@H]1C[C@@H](O)[C@@H](CO)O1 ; O=C(Nc1ccc2ccccc2c1)Nc1ncnc2c1[nH]c(=O)n2[C@H]1C[C@@H](O)[C@@H](CO)O1]

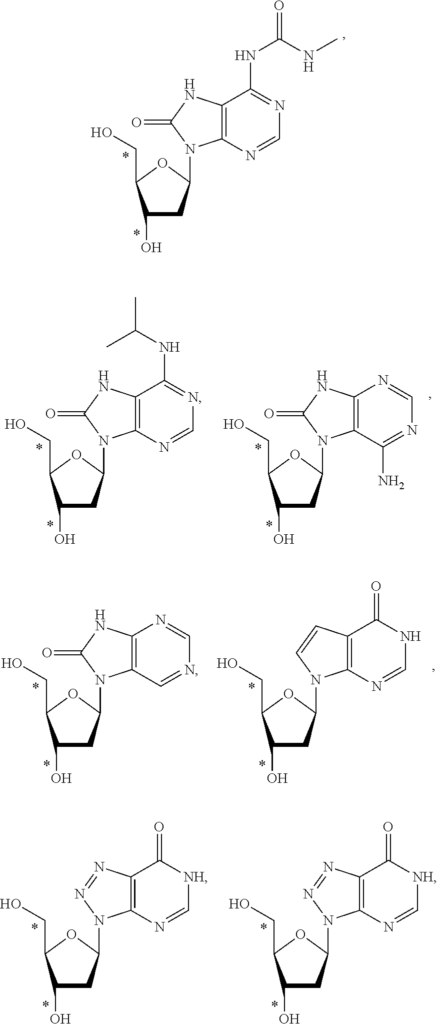

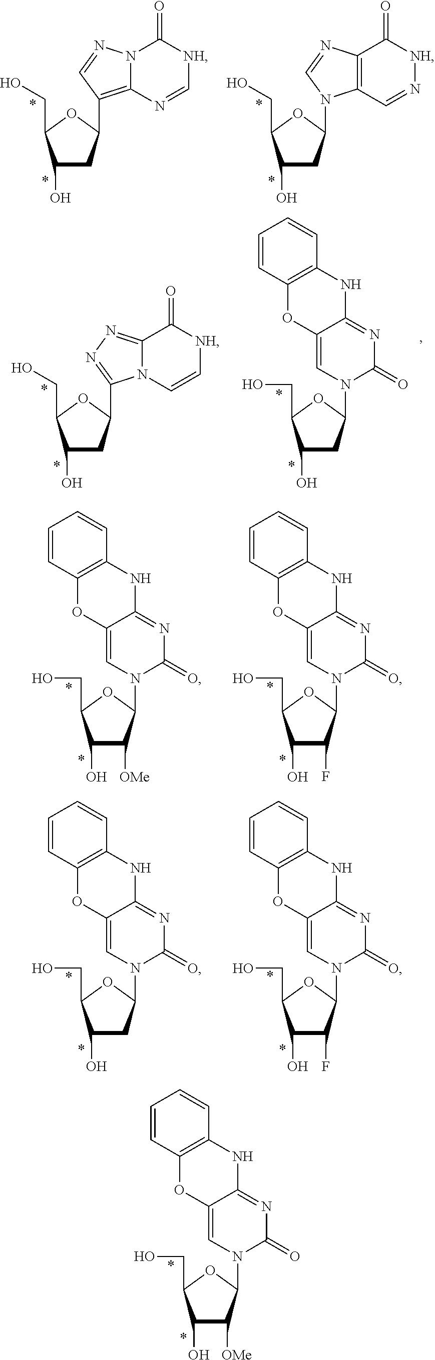

or a salt thereof, wherein “*” indicates connection to internucleotidic linkages when in various oligonucleotides. In some embodiments, an oligonucleotide comprises a nucleoside described herein. In some embodiments, a nucleoside is connected to a internucleotidic linkage through a nitrogen atom (e.g., sm01, sml8, etc.), wherein the nitrogen atom is directly connected to a linkage phosphorus atom. In some embodiments, the present disclosure provides monomers of nucleosides (e.g., Asm01, Gsm01, Tsm18, etc.) as described herein. In some embodiments, the present disclosure provides phosphoramidites of nucleosides as described herein. In some embodiments, such monomers or phosphoramidites comprise protected hydroxyl (e.g., DMTrO—) and/or protected nucleobases (e.g., for oligonucleotide synthesis). In some embodiments, such monomers or phosphoramidites comprise protected hydroxyl (e.g., DMTrO—), optionally protected nucleobases (e.g., as useful for oligonucleotide synthesis), and/or chiral auxiliary groups. Certain reagents, such as various phosphoramidites, that are useful for incorporating various nucleosides and/or compounds into oligonucleotides, and certain technologies for utilizing such reagents for oligonucleotide preparation, e.g., cycles, conditions, etc., are described in the Examples or WO 2021/071858, WO 2022/099159, and/or PCT/US2023/018742. Certain oligonucleotides comprising modified nucleosides and compositions thereof are prepared utilizing such reagents and technologies and are presented herein as examples, e.g., those in various Tables including those in Table 1.

In some embodiments, the present disclosure provides oligonucleotides comprising one or more modified nucleobases as described herein. In some embodiments, the present disclosure provides compounds comprising modified nucleobases as described herein. In some embodiments, the present disclosure provides monomers (e.g., those useful for oligonucleotide synthesis) comprising modified nucleobases as described herein. In some embodiments, the present disclosure provides phosphoramidites comprising modified nucleobases as described herein. In some embodiments, phosphoramidites are CED phosphoramidites. In some embodiments, monomers comprise auxiliary moieties as described herein (e.g., with P forming bonds to O and N, to O and S, to S and S, etc.). In some embodiments, phosphoramidites comprise chiral auxiliary moieties as described herein (e.g., with P forming bonds to O and N). In some embodiments, R^{NS} comprises a nucleobase as described herein. In some embodiments, R^{NS} comprises a modified nucleobase as described herein. In some embodiments, nucleobases are protected for oligonucleotide synthesis.

In some embodiments, an oligonucleotide comprises one or more structures independently selected from pseudoisocytidine, Benner's base Z. 5-hydroxyC, 5-aminoC and 8-oxoA.

In some embodiments, a nucleobase is optionally substituted 2AP (2-amino purine,

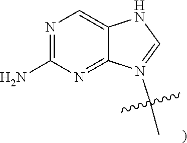

or DAP (2,6-diamino purine,

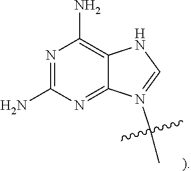

In some embodiments, a nucleobase is optionally substituted 2AP. In some embodiments, a nucleobase is optionally substituted DAP. In some embodiments, a nucleobase is 2AP. In some embodiments, a nucleobase is DAP.

As appreciated by those skilled in the art, various nucleobases are known in the art and can be utilized in accordance with the present disclosure, e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the sugar, base, and internucleotidic linkage modifications of each of which are independently incorporated herein by reference. In some embodiments, nucleobases are protected and useful for oligonucleotide synthesis.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided oligonucleotide comprises one or more 5-methylcytosine. In some embodiments, the present disclosure provides an oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide —such oligonucleotide comprises a nucleobase modification at the C position (e.g., see various oligonucleotides in Table 1). In description of oligonucleotides, typically unless otherwise noted, nucleobases, sugars and internucleotidic linkages are non-modified.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:
a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
one or more atoms of a nucleobase are independently replaced with a different atom selected from carbon, nitrogen and sulfur;
one or more double bonds in a nucleobase are independently hydrogenated; or one or more aryl or heteroaryl rings are independently inserted into a nucleobase.

In some embodiments, a base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-RI are optionally taken together with their intervening atoms to form a 3-30 membered bicyclic or polycyclic ring having 0-10 heteroatom ring atoms. In some embodiments, a modified base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms, wherein the modified base is different than the natural A, T, C, G and U. In some embodiments, a base is optionally substituted A, T, C, G or U. In some embodiments, a modified base is substituted A, T, C, G or U, wherein the modified base is different than the natural A, T, C, G and U.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added. Certain examples of modified nucleobases, including nucleobase replacements, are described in the Glen Research catalog (Glen Research, Sterling, Virginia); Krueger A T et al., Acc. Chem. Res., 2007, 40, 141-150; Kool, ET, Acc. Chem. Res., 2002, 35, 936-943; Benner S.A., et al., Nat. Rev. Genet., 2005, 6, 553-543; Romesberg, F. E., et al., Curr. Opin. Chem. Biol., 2003, 7, 723-733; or Hirao, I., Curr. Opin. Chem. Biol., 2006, 10, 622-627. In some embodiments, an expanded-size nucleobase is an expanded-size nucleobase described in, e.g., WO2017/210647. In some embodiments, modified nucleobases are moieties such as corrin- or porphyrin-derived rings. Certain porphyrin-derived base replacements have been described in, e.g., Morales-Rojas, H and Kool, ET, Org. Lett., 2002, 4, 4377-4380. In some embodiments, a porphyrin-derived ring is a porphyrin-derived ring described in, e.g., WO2017/219647. In some embodiments, a modified nucleobase is a modified nucleobase described in, e.g., WO2017/219647. In some embodiments, a modified nucleobase is fluorescent. Examples of such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, naphtho-uracil, etc., and those described in e.g., WO2017/210647. In some embodiments, a nucleobase or modified nucleobase is selected from: C5-propyne T, C5-propyne C, C5-Thiazole, phenoxazine, 2-thio-thymine, 5-triazolylphenyl-thymine, diaminopurine, and N2-aminopropylguanine.

In some embodiments, a modified nucleobase is selected from 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-rⁱbosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2- one (G-clamp). In some embodiments, modified nucleobases are those in which the purine or pyrimidine base is replaced with other heterocycles, for example, 7-deaza-adenine, 7-deazaguanosine, 2-aminopyridine or 2-pyridone. In some embodiments, modified nucleobases are those disclosed in U.S. Pat. No. 3,687,808, The Concise Encyclopedia Of Polymer Science And Engineering, Kroschwitz, J.I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; or in Chapters 6 and 15, Antisense Drug Technology, Crooke S.T., Ed., CRC Press, 2008, 163-166 and 442-443.

In some embodiments, modified nucleobases and methods thereof are those described in US 20030158403, U.S. Pat. Nos. 3,687,808, 4,845,205, 5,130,302, 5,134,066, 5,175,273, 5,367,066, 5,432,272, 5,434,257, 5,457,187, 5,459,255, 5,484,908, 5,502,177, 5,525,711, 5,552,540, 5,587,469, 5,594,121, 5,596,091, 5,614,617, 5,645,985, 5,681,941, 5,750,692, 5,763,588, 5,830,653, or 6,005,096.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethvl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonvlmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

Certain examples of nucleobases and related methods are described in U.S. Pat. Nos. 3,687,808, 4,845,205, 513,030, 5,134,066, 5,175,273, 5,367,066, 5,432,272, 5,457,187, 5,457,191, 5,459,255, 5,484,908, 5,502,177, 5,525,711, 5,552,540, 5,587,469, 5,594,121, 5,596,091, 5,614,617, 5,681,941, 5,750,692, 6,015,886, 6,147,200, 6,166,197, 6,222,025, 6,235,887, 6,380,368, 6,528,640, 6,639,062, 6,617,438, 7,045,610, 7,427,672, or 7,495,088.

In some embodiments, an oligonucleotide comprises a nucleobase, sugar, nucleoside, and/or internucleotidic linkage which is described in any of Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; or WO 2016/079181.

In some embodiments, an oligonucleotide comprises a modified nucleobase, nucleoside or nucleotide which is described in any of: Feldman et al. 2017 J. Am. Chem. Soc. 139: 11427-11433, Feldman et al. 2017 Proc. Natl. Acad. Sci. USA 114: E6478-E6479, Hwang et al. 2009 Nucl. Acids Res. 37: 4757-4763, Hwang et al. 2008 J. Am. Chem. Soc. 130: 14872-14882, Lavergne et al. 2012 Chem. Eur. J. 18: 1231-1239, Lavergne et al. 2013 J. Am. Chem. Soc. 135: 5408-5419, Ledbetter et al. 2018 J. Am. Chem. Soc. 140: 758-765, Malyshev et al. 2009 J. Am. Chem. Soc. 131: 14620-14621, Seo et al. 2009 Chem. Bio. Chem. 10: 2394-2400, e.g., d3FB, d2Py analogs, d2Py, d3MPy, d4MPy, d5MPy, d34DMPy, d35DMPy, d45DMPy, d5FM, d5PrM, d5SICS, dFEMO, dMMO2, dNaM, dNM01, dTPT3, nucleotides with 2′-azido, 2′-chloro, 2′-amino or arabinose sugars, isocarbostiryl-, napthyl- and azaindole-nucleotides, and modifications and derivatives and functionalized versions thereof, e.g., those in which the sugar comprises a 2′-modification and/or other modification, and dMMO2 derivatives with meta-chlorine, -bromine, -iodine, -methyl, or -propinyl substituents.

In some embodiments, a nucleobase comprises at least one optionally substituted ring which comprises a heteroatom ring atom. In some embodiments, a nucleobase comprises at least one optionally substituted ring which comprises a nitrogen ring atom. In some embodiments, such a ring is aromatic. In some embodiments, a nucleobase is bonded to a sugar through a heteroatom. In some embodiments, a nucleobase is bonded to a sugar through a nitrogen atom. In some embodiments, a nucleobase is bonded to a sugar through a ring nitrogen atom.

In some embodiments, an oligonucleotide comprises a nucleobase or modified nucleobase as described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the bases and modified nucleobases of each of which are independently incorporated herein by reference.

In some embodiments, a nucleobase is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, a provided oligonucleotide comprises a modified nucleobase described in, e.g., U.S. Pat. Nos. 5,552,540, 6,222,025, 6,528,640, 4,845,205, 5,681,941, 5,750,692, 6,015,886, 5,614,617, 6,147,200, 5,457,187, 6,639,062, 7,427,672, 5,459,255, 5,484,908, 7,045,610, 3,687,808, 5,502,177, 5,525,711 6235887, U.S. Pat. Nos. 5,175,273, 6,617,438, 5,594,121, 6,380,368, 5,367,066, 5,587,469, 6,166,197, 5,432,272, 7,495,088, 5,134,066, or U.S. Pat. No. 5,596,091. In some embodiments, a nucleobase is described in WO 2020/154344, WO 2020/154343, WO 2020/154342, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256, and can be utilized in accordance with the present disclosure.

In some embodiments, a nucleobase is a protected base residue as used in oligonucleotide preparation. In some embodiments, a nucleobase is a base residue illustrated in US 2011/0294124, US 2015/0211006, US 2015/0197540, WO 2015/107425, WO 2017/192679, WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the base residues of each of which are independently incorporated herein by reference.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

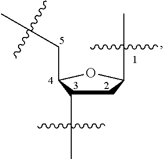

wherein a nucleobase is attached to the 1′ position, and the 3′ and 5′ positions are connected to internucleotidic linkages (as appreciated by those skilled in the art, if at the 5′-end of oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

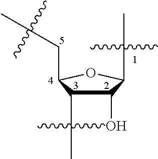

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target nucleic acid recognition. In some embodiments, modified sugars can be utilized to optimize Tm. In some embodiments, modified sugars can be utilized to improve oligonucleotide activities.

Various non-natural RNA sugars, such as natural DNA sugar, various modified sugars, etc., may be utilized in accordance with the present disclosure. For example, one or more natural DNA sugars can be tolerated at various positions. In some embodiments, incorporation of one or more natural DNA sugars provides increased levels of editing, or increased levels of editing by ADAR1 (p110, p150 or both), ADAR2, or both. In some embodiments, editing by ADAR1 is improved. In some embodiments, one or more sugars of N₋₃, N₋₁, N₁, N₄, N₅, N₇, N₈, N₁₀, N₁₂, N₁₃, N₁₄, N₁₅, N₁₆, N₁₇, N₁₈, N₂₀, and N₂₁ is independently a natural DNA sugar (—(e.g., N₋₁): counting from No to the 3′-end of an oligonucleotide; + or just a number (e.g., Ni): counting from N₀ to the 5′-end of an oligonucleotide; each N_{NZ} is independently a nucleoside, wherein NZ is an integer from, e.g., about −100, −90, −80, −70, −60, −50, −40, −30, −20, −10, −9, −8, −7, −6, −5, −4, etc. to ). In some embodiments, one or more sugars of N₋₃, N₋₁, N₀, N₁, N₄, N₅, N₇, N⁸, N₁₀, N₁₂, N₁₃, N₁₄, N₁₅, N₁₆, N₁₇, N₁₈, N₂₀. and N₂₁ is independently a natural DNA sugar. In some embodiments, one or more sugars of N₋₁, N₅, N₁₁, N₁₂ and N₂₀ are independently a natural DNA sugar. In some embodiments, a sugar of N₋₁ is a natural DNA sugar. In some embodiments, a sugar of N₀ is a natural DNA sugar. In some embodiments, a sugar of N₁ is a natural DNA sugar. In some embodiments, a sugar of N₅ is a natural DNA sugar. In some embodiments, a sugar of N₁₁ is a natural DNA sugar. In some embodiments, a sugar of N₁₂ is a natural DNA sugar. In some embodiments, modified sugars are tolerated at one or more positions. In some embodiments, 2′-modified sugars, e.g., 2′-F and/or 2′-OR modified sugars are utilized at one or more or a majority of positions, wherein R is optionally substituted C₁₋₆ aliphatic (e.g., methyl). In some embodiments, modified sugars are utilized at one or more or a majority of or all positions out of 5′-N₁N₀N₋₁-3′. In some embodiments, 2′-OR modified sugars are utilized at one or more or a majority of or all positions out of 5′-N₁N₀N₋₁-3′ wherein R is optionally substituted C₁₋₆ aliphatic (e.g., methyl). In some embodiments, modified sugars are utilized at one or more or a majority of or all positions out of 5′-N₁N₀N₋₁-3′ and one or more 2′-F modified sugars, natural DNA sugars and/or natural RNA sugars are utilized in 5′-N₁N₀N₋₁-3′. In some embodiments, modified sugars are utilized at one or more or a majority of or all positions out of 5′-N₁N₀N₋₁-3′ and each sugar of 5′-N₁N₀N₋₁-3′ is independently a 2′-F modified sugar, a natural DNA sugar or a natural RNA sugar. In some embodiments, modified sugars are utilized at one or more or a majority of or all positions out of 5′-N₁N₀N₋₁-3′ and each sugar of 5′-N₁N₀N₋₁-3′ is independently a 2′-F modified sugar or a natural DNA sugar. In some embodiments, modified sugars are utilized at one or more or a majority of or all positions out of 5′-N₁N₀N₋₁-3′ and each sugar of 5′-N₁N₀N₋₁-3′ is independently a natural DNA sugar. In some embodiments, modified sugars, e.g., 2′-OR modified sugars (wherein R is optionally substituted C₁₋₆ alkyl) provide increased levels of editing, or increased levels of editing by ADAR1 (p110, p150 or both), ADAR2, or both. In some embodiments, editing by ADAR2 is improved. In some embodiments, a modified sugar is a bicyclic sugar (e.g., a LNA sugar, a cEt sugar, etc.). In some embodiments, a bicyclic sugar may be utilized at one or more or all positions where a 2′-OR sugar is utilized, wherein R is optionally substituted C₁₋₆ alkyl. In some embodiments, 2′-OR is 2′-OMe. In some embodiments, 2′-OR is 2′-MOE. In some embodiments, a majority is at least 50%, 60%, 70%, 75%, 80%, 85%, 90%, 95%, or 99% (e.g., 55%-100%, 60%-100%, 70-100%, 75%-100%, 80%-100%, 90%-100%, 95%-100%, 60%-95%, 70%-95%, 75-95%, 80-95%, 85-95%, 90-95%, 51%, 60%, 70%, 75%, 80%, 85%, 90%, 95%, or 99%, etc.

In some embodiments, one or more (e.g., 1-10, 1, 2, 3, 4, or 5, etc.) of the first several (e.g., 1-10, 1, 2, 3, 4, or 5, etc.) sugars (unless otherwise specified, from the 5′-end) of a provided oligonucleotide or of a first domain are independently modified sugars. In some embodiments, each of the first several sugars is independently a modified sugar. In some embodiments, the first one, two or three sugars of a provided oligonucleotide or of a first domain are independently modified sugars. In some embodiments, the first sugar is a modified sugar. In some embodiments, the first two sugars are independently modified sugars. In some embodiments, the first three sugars are independently modified sugars. In some embodiments, a modified sugar is a bicyclic sugar. In some embodiments, a modified sugar is a 2′-modified sugar. In some embodiments, each modified sugar is independently a 2′-modified sugar. In some embodiments, a modified sugar is a 2′-OMe modified sugar. In some embodiments, each modified sugar is a 2′-OMe modified sugar. In some embodiments, a modified sugar is a 2′-MOE modified sugar. In some embodiments, each modified sugar is a 2′-MOE modified sugar. In some embodiments, each modified sugar is independently a 2′-OMe or 2′-MOE modified sugar.

In some embodiments, one or more (e.g., 1-10, 1, 2, 3, 4, or 5, etc.) of the last several (e.g., 1-10, 1, 2, 3, 4, or 5, etc.) sugars (unless otherwise specified, from the 5′-end) of a provided oligonucleotide or of a second domain or a third subdomain are independently modified sugars. In some embodiments, each of the last several sugars is independently a modified sugar. In some embodiments, the last one, two or three sugars of a provided oligonucleotide or of a second domain or a third subdomain are independently modified sugars. In some embodiments, the last sugar is a modified sugar. In some embodiments, the last two sugars are independently modified sugars. In some embodiments, the last three sugars are independently modified sugars. In some embodiments, the last four sugars are independently modified sugars. In some embodiments, a modified sugar is a bicyclic sugar. In some embodiments, a modified sugar is a 2′-modified sugar. In some embodiments, each modified sugar is independently a 2′-modified sugar. In some embodiments, a modified sugar is a 2′-OMe modified sugar. In some embodiments, each modified sugar is a 2′-OMe modified sugar. In some embodiments, a modified sugar is a 2′-MOE modified sugar. In some embodiments, each modified sugar is a 2′-MOE modified sugar. In some embodiments, each modified sugar is independently a 2′-OMe or 2′-MOE modified sugar.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position and another sugar at the 3′ position unless otherwise indicated.

In some embodiments, a sugar is an optionally substituted natural DNA or RNA sugar. In some embodiments, a sugar is optionally substituted

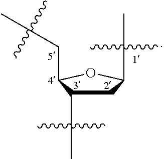

In some embodiments, the 2′ position is optionally substituted. In some embodiments, a sugar is

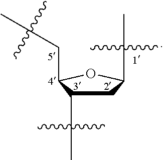

In some embodiments, a sugar has the structure of

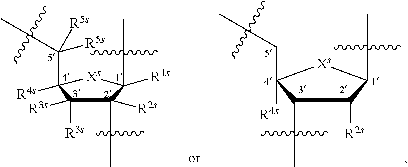

wherein each of R^{1s}, R^{2s}, R^{3s}, R^{4s}, and R^{5S} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′, -L^{s}-OR′, -L^{s}-SR′, -L^{s}-N(R′)₂, —O-L^{s}-OR′, —O-L^{s}-SR′, or -O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two Rs are taken together to form a bridge -L^{s}-. In some embodiments, R′ is optionally substituted C₁₋₁₀ aliphatic. In some embodiments, a sugar has the structure of

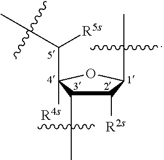

In some embodiments, a sugar has the structure of

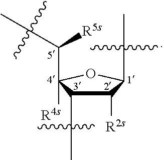

In some embodiments, a sugar has the structure of

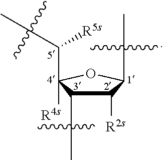

In some embodiments, a sugar has the structure of

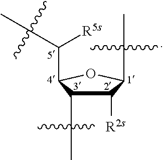

In some embodiments, a sugar has the structure of

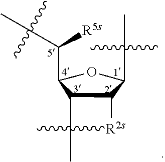

In some embodiments, a sugar has the structure of

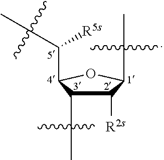

In some embodiments, a sugar has the structure of

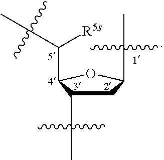

In some embodiments, a sugar has the structure of

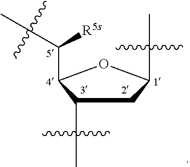

In some embodiments, a sugar has the structure of

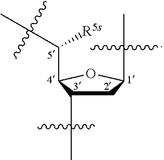

In some embodiments, R^{5s} is optionally substituted C₁₋₆ aliphatic. In some embodiments, R^{5s} is optionally substituted C₁₋₆ alkyl. In some embodiments, R^{5s} is optionally substituted methyl. In some embodiments, R^{5s} is methyl. In some embodiments, a sugar has the structure of

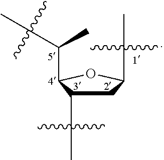

In some embodiments, a sugar has the structure of

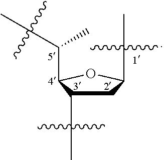

In some embodiments, a sugar has the structure of

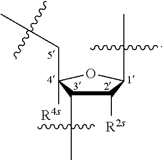

Various such sugars are utilized in Table 1. In some embodiments, a sugar has the structure of

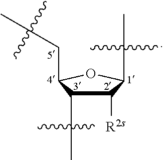

In some embodiments, a 2′-modified sugar has the structure of

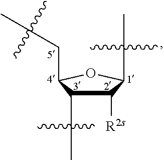

wherein R^{2s} is a 2′-modification. In some embodiments, a sugar has the structure of

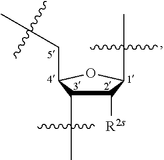

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, a modified nucleoside is mA, mT, mC, m5mC, mG, mU, etc., in which R^{2s} is —OMe. In some embodiments, R^{2s} is —OCH₂CH₂OMe. In some embodiments, a modified nucleoside is Aeo, Teo, Ceo, m5Ceo, Geo, Ueo, etc., in which R^{2s} is —OCH₂CH₂OMe. In some embodiments, R^{2s} is —OCH₂CH₂OH. In some embodiments, an oligonucleotide comprises a 2′-F modified sugar having the structure of

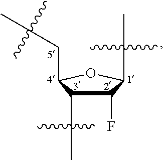

(e.g., as in fA, fT, fC, f5mC, fG, fU, etc.). In some embodiments, an oligonucleotide comprises a 2′-OMe modified sugar having the structure of

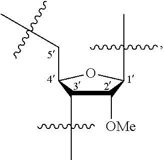

(e.g., as in mA, mT, mC, m5mC, mG, mU, etc.). In some embodiments, an oligonucleotide comprises a 2′-MOE modified sugar having the structure of

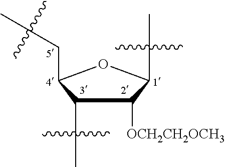

(e.g., as in Aco, Teo, Ceo, m5Ceo, Geo, Ueo, etc.).

In some embodiments, a sugar has the structure of

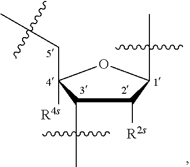

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C₂—O—CH₂—C4. In some embodiments, L^{s} is C2-O—CH₂—C4. In some embodiments, L^{s} is C2-O—(R)—CH(CH₂CH₃)—C4. In some embodiments, L^{S} is C2-O—(S)—CH(CH₂CH₃)—C4.

In some embodiments, a sugar has the structure of

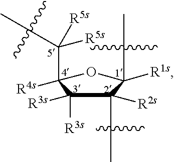

wherein each variable is independently as described herein. In some embodiments, a sugar has the structure of

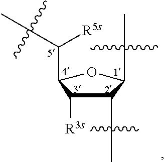

wherein each variable is independently as described herein. In some embodiments, R^{5s} is —H. In some embodiments, a sugar has the structure of

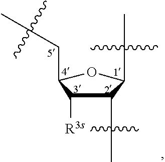

wherein each variable is independently as described herein. In some embodiments, R^{3s} is —OH. In some embodiments, R^{3s} is —H. In some embodiments, a sugar is

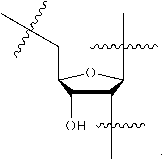

In some embodiments, a sugar is

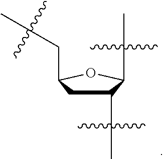 [SMILES: *C[C@@H]1C[C@H](*)[C@H](*)O1]

In some embodiments, a sugar is optionally substituted

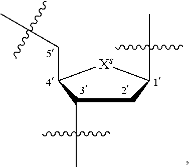

wherein X^{s} is —S—, —Se—, or optionally substituted —CH₂—. In some embodiments, the 2′ position is optionally substituted. In some embodiments, a sugar is

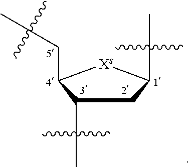

In some embodiments, a sugar has the structure of

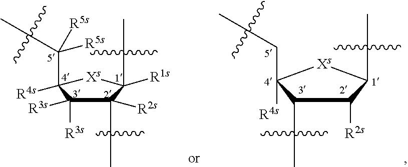

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the substituents, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′, -L^{s}-OR′, -L^{s}-SR′, -L^{s}-N(R′)₂, —O-L^{s}-OR′, —O-L^{s}-SR′, or —O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two R^{s} are taken together to form a bridge -L^{s}-. In some embodiments R′ is optionally substituted C₁₋₁₀ aliphatic. In some embodiments, a sugar has the structure of

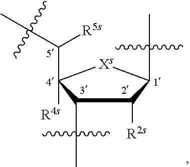

In some embodiments, a sugar has the structure of

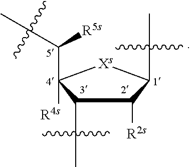

In some embodiments, a sugar has the structure of

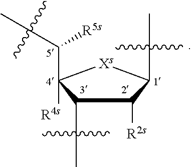

In some embodiments, a sugar has the structure of

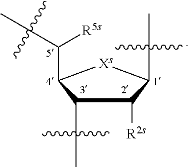

In some embodiments, a sugar has the structure of

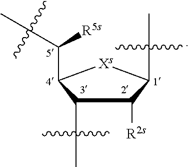

In some embodiments, a sugar has the structure of

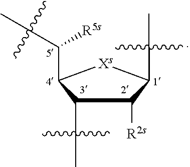

In some embodiments, a sugar has the structure of

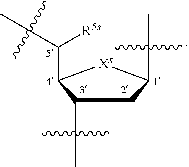

In some embodiments, a sugar has the structure of

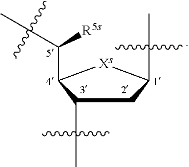

In some embodiments, a sugar has the structure of

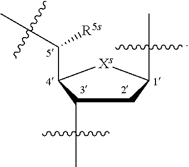

In some embodiments, R^{5s} is optionally substituted C₁₋₆ aliphatic. In some embodiments, R^{5s} is optionally substituted C₁₋₆ alkyl. In some embodiments, R^{5S} is optionally substituted methyl. In some embodiments, R^{5s} is methyl. In some embodiments, a sugar has the structure of

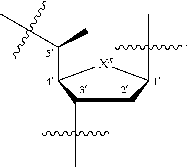

In some embodiments, a sugar has the structure of

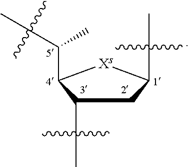

In some embodiments, a sugar has the structure of

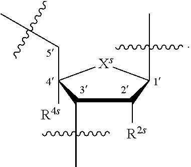

Various such sugars are utilized in Table 1. In some embodiments, a sugar has the structure of

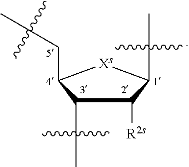

In some embodiments, a 2′-modified sugar has the structure of

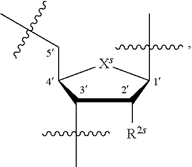

wherein R^{2s} is a 2′-modification. In some embodiments, a sugar has the structure of

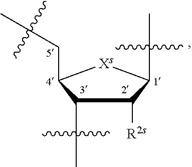

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, R^{2s} is —OCH₂CH₂OMe. In some embodiments, R^{2s} is —OCH₂CH₂OH. In some embodiments, a modified sugar has the structure of

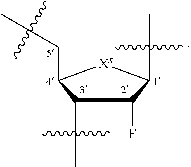

In some embodiments, a modified sugar has the structure of

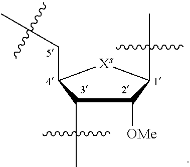

In some embodiments, a modified sugar having the structure of

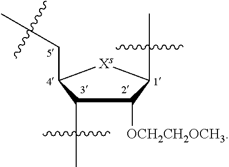

In some embodiments, a modified sugar having the structure of

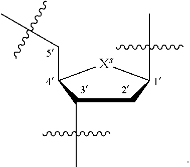

In some embodiments, X^{s} is —S—. In some embodiments, X^{s} is optionally substituted —CH₂—. In some embodiments, X^{s} is —CH₂—. In some embodiments, a modified sugar having the structure of

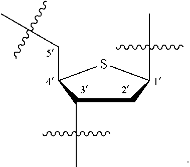 [SMILES: *C[C@H]1S[C@@H](*)C[C@H]1*]

In some embodiments, a modified sugar having the structure of

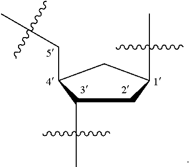 [SMILES: *C[C@H]1C[C@@H](*)C[C@H]1*]

In some embodiments, a sugar has the structure of

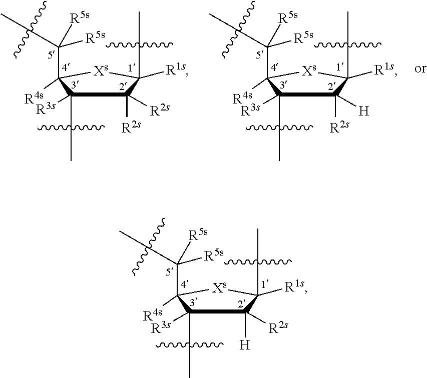

wherein each R^{2s} is independently —H, —F, —OH or —OR^{ak}, wherein R^{ak} is optionally substituted C₁₋₆ aliphatic, and each of the other variables is independently as described herein. In some embodiments, each of R^{1s}, R^{3s}, R^{4s}, and R^{5s} is independently —H. In some embodiments, each of R^{1s}, R^{3s} and R^{4s}, and one of R^{5s}, are independently —H, and the other R^{5s} is independently C₁₋₆ aliphatic. In some embodiments, an occurrence of R^{5s} is C₁₋₆ aliphatic, e.g., methyl. In some embodiments, R^{2s} is —H. In some embodiments, R^{2s} is —F. In some embodiments, R^{2s} is —OR^{ak}. In some embodiments, R^{2s} is —OMe. In some embodiments, R^{2s} is —OCH₂CH₂CH₃. In some embodiments, at least one occurrence of R^{2s} is —H. In some embodiments, at least one occurrence of R^{2s} is not —H. In some embodiments, X^{s} is —O—. In some embodiments, X^{s} is —S—. In some embodiments, X^{s} is optionally substituted —CH₂—. In some embodiments, X^{s} is —CH₂—.

In some embodiments, a sugar has the structure of

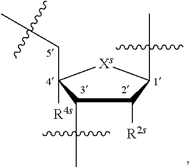

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4. In some embodiments, X^{s} is —S—. In some embodiments, X^{s} is optionally substituted —CH₂—. In some embodiments, X^{s} is —CH₂—. In some embodiments, X^{s} is —Se—.

In some embodiments, a sugar has the structure of

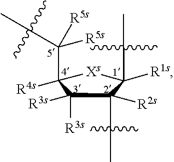

wherein each variable is independently as described herein. In some embodiments, a sugar has the structure of

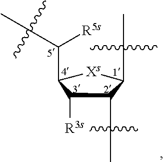

wherein each variable is independently as described herein. In some embodiments, R^{5s} is —H. In some embodiments, a sugar has the structure of

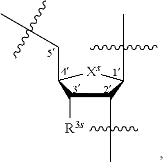

wherein each variable is independently as described herein. In some embodiments, R^{3s} is —OH. In some embodiments, R^{3s} is —H. In some embodiments, X^{s} is —S—. In some embodiments, X^{s} is optionally substituted —CH₂—. In some embodiments, X^{s} is —CH₂—.

In some embodiments, a nucleoside comprising a modified sugar has the structure of

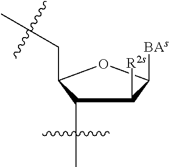

or a salt form thereof, wherein BA^{s} is —H or an optionally substituted or protected nucleobase (e.g., BA), and R^{2s} is as described herein. In some embodiments, R^{2s} is —OH, halogen, or optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, R^{2s} is —F. In some embodiments, a nucleoside comprising a modified sugar has the structure of

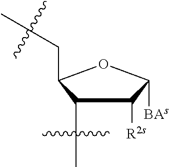

or a salt form thereof, wherein each variable is independently as described herein. In some embodiments, R^{2S} is —H, —OH, halogen, or optionally substituted C₁-C₆ alkoxy. In some embodiments, R^{2s} is —H. In some embodiments R^{2s}is —F. In some embodiments, a nucleoside comprising a modified sugar has the structure of

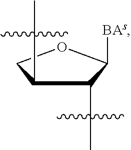

wherein each variable is as described herein. In some embodiments, a nucleoside comprising a modified sugar has the structure of

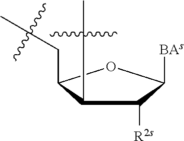

or a salt form thereof, wherein each variable is independently as described herein. In some embodiments, R^{2s} is —H, —OH, halogen, or optionally substituted C₁-C₆ alkoxy. In some embodiments, R^{2s} is —H. In some embodiments, R^{2s} is —F. In some embodiments, a nucleoside comprising a modified sugar has the structure of

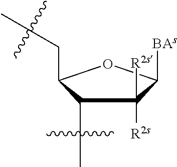

or a salt form thereof, wherein R^{2s}′ is R^{s}, and each of R^{s}, R^{2s} and BA^{s} is independently as described herein. In some embodiments, each of R^{2s} and R^{2s}′ is independently —H, —OH, halogen, or optionally substituted C₁-C₆ alkoxy. In some embodiments, R^{2s} is —H. In some embodiments, R^{2s} is —OH. In some embodiments, R^{2s} is halogen. In some embodiments, R^{2s} is —F. In some embodiments, R^{2s} is optionally substituted C₁-C₆ alkoxy. In some embodiments, R^{2s} is —H. In some embodiments, R^{2s}′ is —OH. In some embodiments, R^{2s}′ is halogen. In some embodiments, R^{2s} is —F. In some embodiments, R^{2s}′ is optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides including sugars and nucleobases and uses thereof are described in WO 2020/154342. In some embodiments, an oligonucleotide comprises arabinoside, 2′-deoxy-2′-fluoro-arabinoside, 2′-OR arabinoside, adeoxycytidine, DNA-abasic, RNA-abasic, or 2′-OR abasic, wherein R is not hydrogen (e.g., optionally substituted C₁₋₆ aliphatic). In some embodiments, 2′-OR is 2′-OMe. In some embodiments, 2′-OR is 2′-MOE. In some embodiments, an oligonucleotide comprises 2′-O-methyl-arabinocytidine (amC). In some embodiments, oligonucleotides comprise such nucleosides. In some embodiments, monomers comprise such nucleosides. In some embodiments, phosphoramidites comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite). In some embodiments, one or more or each of a 5′ immediate nucleoside (e.g., N₁), an opposite nucleoside (N₀) and a 3′ immediate nucleoside (e.g., N₋₁) is independently such a nucleoside. In some embodiments, 5′-N₁N₀N₋₁-3′ is amCCA. In some embodiments, a sugar has the structure of

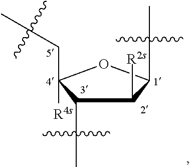

wherein each variable is as described herein and C1′ is bonded to a nucleobase. In some embodiments, a sugar is an arabinose. In some embodiments, a sugar has the structure of

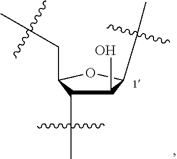

wherein C1′ is bonded to a nucleobase.

In some embodiments, a sugar is optionally substituted

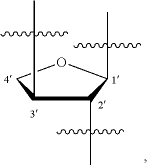

wherein a nucleobase is bonded at position 1′. In some embodiments, a sugar is

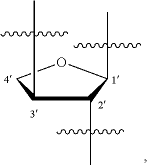

wherein a nucleobase is bonded at position 1′.

In some embodiments, a sugar is optionally substituted

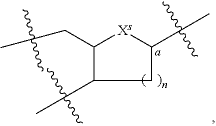

wherein position a is bonded to a nucleobase, X is —O—, —S—, —Sc— or optionally substituted —CH₂—. In some embodiments, a sugar is

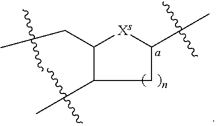

In some embodiments, a sugar is optionally substituted

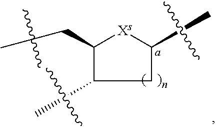

wherein position a is bonded to a nucleobase, X^{s} is —O—, —S—, —Se— or optionally substituted —CH₂—. In some embodiments, a sugar is

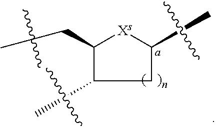

In some embodiments, X^{s} is —O—. In some embodiments, X^{s} is —S—. In some embodiments, X^{s} is —Se—. In some embodiments, X^{s} is optionally substituted —CH₂—. In some embodiments, X^{s} is —CH₂—. In some embodiments, n is 0. In some embodiments, n is 1. In some embodiments, n is 2. In some embodiments, n is 3.

In some embodiments, a modified sugar comprises an optionally substituted 6-membered ring having 0-1 oxygen atom. In some embodiments, a modified sugar comprises an optionally substituted 6-membered ring having an oxygen atom. For example, in some embodiments, a modified sugar has the structure of optionally substituted

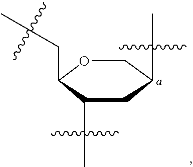 [SMILES: *C[C@H]1OC[C@@H](*)C[C@H]1*]

wherein position a is bonded to a nucleobase. In some embodiments, a modified sugar has the structure of

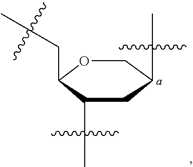 [SMILES: *C[C@H]1OC[C@@H](*)C[C@H]1*]

wherein position a is bonded to a nucleobase. in some embodiments, a modified sugar has the structure of optionally substituted

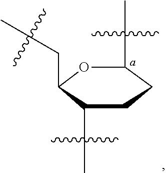

wherein position a is bonded to a nucleobase. In some embodiments, a modified sugar has the structure of

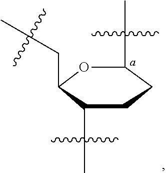

wherein position a is bonded to a nucleobase. In some embodiments, a modified sugar has the structure of

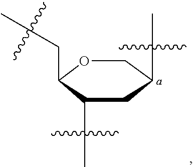 [SMILES: *C[C@H]1OC[C@@H](*)C[C@H]1*]

wherein position a is bonded to a nucleobase. In some embodiments, a modified sugar has the structure of optionally substituted

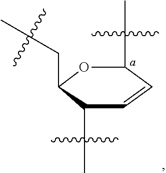

wherein position a is bonded to a nucleobase. In some embodiments, a modified sugar has the structure of

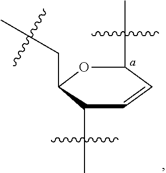

wherein position a is bonded to a nucleobase. In some embodiments, a modified sugar has the structure of optionally substituted

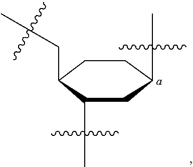 [SMILES: *C[C@H]1CC[C@@H](*)C[C@H]1*]

wherein position a is bonded to a nucleobase. In some embodiments, a modified sugar has the structure of

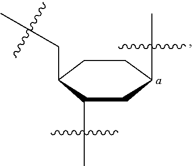 [SMILES: *C[C@H]1CC[C@@H](*)C[C@H]1*]

wherein position a is bonded to a nucleobase. In some embodiments, a modified sugar has the structure of optionally substituted

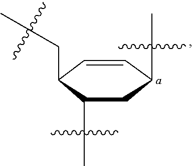 [SMILES: *C[C@H]1C=C[C@@H](*)C[C@H]1*]

wherein position a is bonded to a nucleobase. In some embodiments, a modified sugar has the structure of

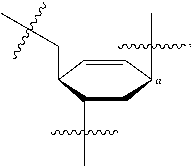 [SMILES: *C[C@H]1C=C[C@@H](*)C[C@H]1*]

wherein position a is bonded to a nucleobase.

In some embodiments, a nucleoside comprising a modified sugar has the structure of

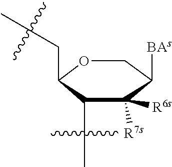

or a salt form thereof, wherein each of R^{6s} and R^{7s} is independently Rs, BA^{s} is —H or an optionally substituted or protected nucleobase (e.g., BA), and R^{s} is independently as described herein. In some embodiments, R^{6s} is —H, —OH or halogen, and R^{7s} is —H, —OH, halogen or optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, a nucleoside comprising a modified sugar has the structure of

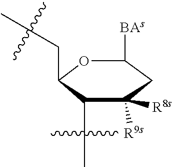

or a salt form thereof, wherein each of R^{8s} and R^{9s} is independently R^{s}, and each of R^{s} and BA^{s} is independently as described herein. In some embodiments, R^{8s} is —H or halogen, and R^{9s} is —H, —OH, halogen, or optionally substituted C₁-C₆ alkoxy. In some embodiments, a nucleoside comprising a modified sugar has the structure of

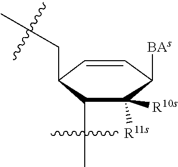

or a salt form thereof, wherein each of R^{10s} and R^{11s} is independently R^{s}, and each of R^{s} and BA^{s} is independently as described herein. In some embodiments, R^{10s} is —H or halogen, and R^{11s} is —H, —OH, halogen, or optionally substituted C₁-C₆ alkoxy. In some embodiments, a nucleoside comprising a modified sugar has the structure of

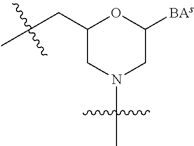

or a salt form thereof, wherein BA^{s} is as described herein. In some embodiments, a nucleoside comprising a modified sugar has the structure of

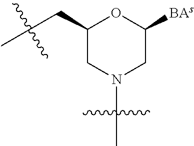

or a salt form thereof, wherein BA^{s} is as described herein. Those skilled in the art appreciate that in some embodiments, the nitrogen may be directly bonded to linkage phosphorus. In some embodiments, a halogen is —F. In some embodiments, BA^{s} is —H. In some embodiments, BAS is an optionally substituted or protected nucleobase. In some embodiments, BA^{s} is BA. In some embodiments, nucleobases such as BA are optionally substituted or protected for oligonucleotide synthesis. In some embodiments, an oligonucleotide comprises alpha-homo-DNA, beta-homo-DNA moieties. In some embodiments, an oligonucleotide comprises an alpha- or beta-homo-DNA sugar. In some embodiments, an oligonucleotide comprises an alpha-homo-DNA sugar. In some embodiments, an oligonucleotide comprises a beta-homo-DNA sugar. Certain such nucleosides including sugars and nucleobases and uses thereof are described in WO 2020/154343. In some embodiments, oligonucleotides comprise such nucleosides. In some embodiments, monomers comprise such nucleosides. In some embodiments, phosphoramidites comprise such nucleosides (in some embodiments, one connecting site (e.g., a —CH₂— connecting site) is bonded to an optionally substituted —OH, e.g., —ODMTr, and one connecting site (e.g., a ring connecting site) is bonded to P of a phosphoramidite (e.g., when the connecting ring atom is N) or to O which is also bonded to P of a phosphoramidite(e.g., when the connecting ring atom is C)). In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a modified sugar has the structure of

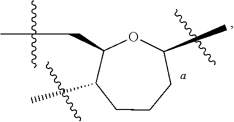

wherein position a is bonded to a nucleobase. In some embodiments, a modified sugar has the structure of

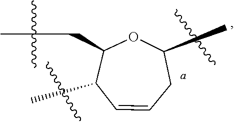

wherein position a is bonded to a nucleobase. In some embodiments, a modified sugar has the structure of

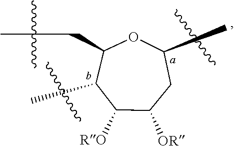

wherein position a is bonded to a nucleobase, position b is bonded to an internucleoside linkage and R″ is —H or optionally substituted C₁₋₆ aliphatic. In some embodiments, a modified sugar has the structure of

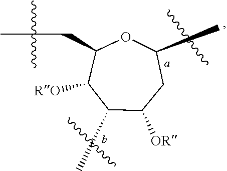

wherein position a is bonded to a nucleobase, position b is bonded to an internucleoside linkage and R″ is —H or C₁₋₆ aliphatic. In some embodiments, a modified sugar has the structure of

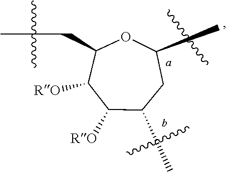

wherein position a is bonded to a nucleobase, position b is bonded to an internucleoside linkage and R″ is —H or C₁₋₆ aliphatic. In some embodiments, R″ is methyl.

In some embodiments, a nucleoside comprising a modified sugar has the structure of

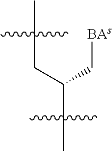

or a salt form thereof, wherein each variable is as described herein. In some embodiments, a nucleoside comprising a modified sugar has the structure of

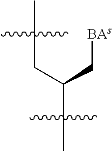

or a salt form thereof, wherein each variable is as described herein. In some embodiments, a nucleoside comprising a modified sugar has the structure of

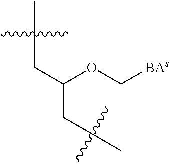

or a salt form thereof, wherein each variable is as described herein. In some embodiments, a nucleoside comprising a modified sugar has the structure of

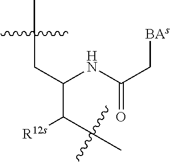

or a salt form thereof, wherein R¹²s is R^{s}, and each of Rs and BA^{s} is independently as described herein. In some embodiments, R^{12s} is —H, —OH, halogen, optionally substituted C₁₋₆ alkyl, optionally substituted C₁₋₆ heteroalkyl, or optionally substituted C₁₋₆ alkoxy. In some embodiments, a halogen is —F. In some embodiments, a nucleoside comprising a modified sugar has the structure of

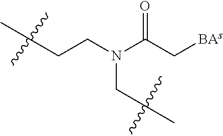

or a salt form thereof, wherein each variable is as described herein. In some embodiments, a nucleotide comprising a modified sugar has the structure of

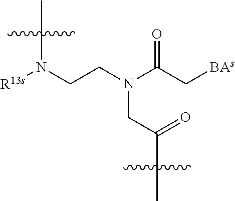

or a salt form thereof, wherein R^{13s} is R^{s}, and each of R^{s} and BA^{s} is independently as described herein. In some embodiments. R^{13s} is —H or optionally substituted C₁-C₆ alkyl. In some embodiments, a nucleoside comprising a modified sugar has the structure of

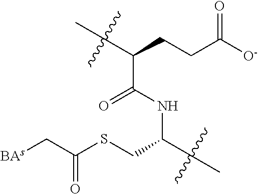

or a salt form thereof, wherein each variable is as described herein. In some embodiments, a nucleotide comprising a modified sugar has the structure of

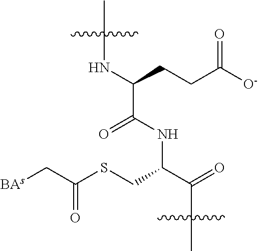

or a salt form thereof, wherein each variable is as described herein. In some embodiments, a linkage is an amide linkage. In some embodiments, BA^{s} is —H. In some embodiments, BA^{s} is an optionally substituted or protected nucleobase. In some embodiments, BAS is BA. In some embodiments, nucleobases such as BA are optionally substituted or protected for oligonucleotide synthesis. Certain such nucleosides and nucleotides including sugars and nucleobases and uses thereof are described in WO 2020/154344. In some embodiments. oligonucleotides comprise such nucleosides. In some embodiments, oligonucleotides comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite. In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a sugar is an acyclic sugar, e.g. a UNA sugar. In some embodiments, a sugar is optionally substituted

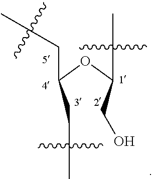

In some embodiments, the 2′ position is optionally substituted.
In some embodiments, a sugar is

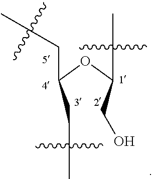

In some embodiments, a sugar has the structure of

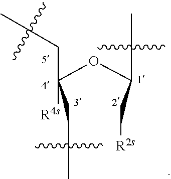

In some embodiments, R^{2s} is —OH. In some embodiments, a sugar is

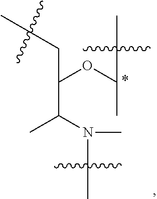 [SMILES: *CC(OC(*)C)C(C)N(*)C]

wherein “*” indicates the carbon atom bonded to a nucleobase. In some embodiments, a sugar is

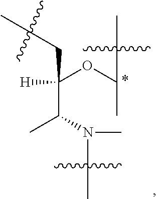 [SMILES: *C[C@@H](OC(*)C)[C@@H](C)N(*)C]

wherein “*” indicates the carbon atom bonded to a nucleobase. In some embodiments, the carbon atom bonded to a nitrogen atom of a nucleobase and is of R consiguration (e.g., sm18). In some embodiments, an oligonucleotide comprises a sugar described herein.

In some embodiments, a sugar is optionally substituted

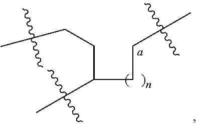

wherein position a is bonded to a nucleobase, X^{s} is —O—, —S—, —Se— or optionally substituted —CH₂—. In some embodiments, a sugar is

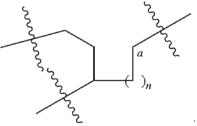

In some embodiments, X^{s} is —O—. In some embodiments, X^{s} is -S-. In some embodiments, X^{s} is —Se—. In some embodiments, X^{s} is optionally substituted —CH₂—. In some embodiments, X^{s} is —CH₂—. In some embodiments, n is 0. In some embodiments, n is 1. In some embodiments, n is 2. In some embodiments, n is 3.

In some embodiments, a sugar is connected not through 5′ and 3′ positions. Those skilled in the art appreciate that for such sugars, 5′ can refer to the side/direction toward 5′-end of an oligonucleotide, and 3′ can refer to the side/direction toward to 3′-end of an oligonucleotide.

In some embodiments, each of R^{1s}, R^{2s}, R^{3s}, R^{4s}, and R^{5s} is independently R^{s}, wherein R^{s} is independently —H, halogen, —CN, —N₃, —NO, —NO₂, -L^{s}-R′, -L^{s}-Si(R′)₃, -L^{s}-OR′, -L^{s}-SR′, -L^{s}-N(R′)₂, —O-L^{s}-R′, —O-L^{s}-Si(R)₃, —O-L^{s}-OR′, —O-L^{s}-SR′, or —O-L^{s}-N(R′)₂; wherein L^{s} is L^{B} as described herein, and each other variable is independently as described herein. In some embodiments, each of R^{1s} and R^{2s} is independently R^{s}. In some embodiments, R^{s} is —H. In some embodiments, R^{S} is not —H. In some embodiments, L^{s} is a covalent bond. In some embodiments, each of R^{2s} and R^{4s} are independently —H, —F, —OR, —N(R)₂. In some embodiments, R^{2s} is —H, —F, —OR, —N(R)₂. In some embodiments, R^{4s} is —H. In some embodiments, R^{2s} and R^{4s} form 2′-O-L^{s}-, wherein L^{s} is optionally substituted C₁₋₆ alkylene. In some embodiments, L^{s} is optionally substituted —CH₂—. In some embodiments, L^{s} is optionally substituted —CH₂-.

In some embodiments, R is hydrogen. In some embodiments, R is not hydrogen. In some embodiments, R is an optionally substituted group selected from C₁₋₁₀ aliphatic, C₁₋₁₀ heteroaliphatic having 1-10 heteroatoms independently selected from oxygen, nitrogen, sulfur, phosphorus and silicon, C₆₋₂₀ aryl, a 5-20 membered heteroaryl ring having 1-10 heteroatoms independently selected from oxygen, nitrogen, sulfur, phosphorus and silicon, and a 3-20 membered heterocyclic ring having 1-10 heteroatoms independently selected from oxygen, nitrogen, sulfur, phosphorus and silicon.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂OCH₃.

In some embodiments, R is optionally substituted phenyl. In some embodiments, R is phenyl.

In some embodiments, R^{2s} is a 2′-modification as described in the present disclosure, and R^{4s} is —H. In some embodiments, R^{2s} is —OR, wherein R is not hydrogen. In some embodiments, R^{2s} is —F. In some embodiments, R^{2s} is —OMe. In some embodiments, R^{2s} is —OCH₂CH₂CH₃, e.g., in various Xₑₒ utilized in Table 1 (X being m5C, T, G, A, etc.). In some embodiments, R^{2s} is selected from —H, —F, and —OR, wherein R is optionally substituted C₁₋₆ alkl. In some embodiments, R^{2s} is selected from —H, —F, and —OMe.

In some embodiments, a sugar is a bicyclic sugar, e.g., sugars wherein R^{2s} and R^{4s} are taken to form an optionally substituted ring as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, a bridge is 2′-L^{a}L^{b}-4′, wherein L^{a} is —O—, —S— or N(R), and L^{b} is an optionally substituted C₁₋₄ bivalent aliphatic chain, e.g., methylene.

In some embodiments, a sugar is a 2′-OMe, 2′-MOE, 2′-F, a LNA (locked nucleic acid) sugar, an ENA (ethylene bridged nucleic acid) sugar, a BNA(NMe) (Methylamino bridged nucleic acid) sugar, 2′-F ANA (2′-F arabinose), alpha-DNA (alpha-D-ribose), 2′/5′ ODN (e.g., 2′/5′ linked oligonucleotide), Inv (inverted sugar, e.g., inverted desoxyribose), AmR (Amino-Ribose), ThioR (Thio-ribose). HNA (hexose nucleic acid), CeNA (cyclohexene nucleic acid), or MOR (Morpholino) sugar.

Those skilled in the art after reading the present disclosure will appreciate that various types of sugar modifications are known and can be utilized in accordance with the present disclosure. In some embodiments, a sugar modification is a 2′-modification (e.g., R^{2s}). In some embodiments, a 2′-modification is 2′-F. In some embodiments, a 2′-modification is 2′-OR, wherein R is not hydrogen. In some embodiments, a 2′-modification is 2′-OR, wherein R is optionally substituted C₁₋₆ aliphatic. In some embodiments, a 2′-modification is 2′-OR, wherein R is optionally substituted C₁₋₆ alkyl. In some embodiments, a 2′-modification is 2′-OMe. In some embodiments, a 2′-modification is 2′-MOE. In some embodiments, a 2′-modification is —O-L^{b}- or -L^{b}-L^{b}- which connects the 2′-carbon of a sugar moiety to another carbon of a sugar moiety. In some embodiments, a 2′-modification is 2′-O-L-4′ or 2′-L-4′ which connects the 2′-carbon of a sugar moiety to the 4′-carbon of a sugar moiety. In some embodiments, a 2′-modification is S-cEt. In some embodiments, a modified sugar is an LNA sugar. In some embodiments, -L^{b}- is —C(R)₂—. In some embodiments, a 2′-modification is (C2-O—C(R)₂—C4), wherein each R is independently as described in the present disclosure. In some embodiments, a 2′-modification is a LNA sugar modification (C2-O—CH₂—C4). In some embodiments, a 2′-modification is (C2-O—CHR—C4), wherein R is as described in the present disclosure. In some embodiments, a 2′-modification is (C2-O—(R)—CHR—C4), wherein R is as described in the present disclosure and is not hydrogen. In some embodiments, a 2′-modification is (C2-O—(S)—CHR—C4), wherein R is as described in the present disclosure and is not hydrogen. In some embodiments, R is optionally substituted C₁₋₆ aliphatic. In some embodiments, R is optionally substituted C₁₋₆ alkyl. In some embodiments, R is unsubstituted C₁₋₆ alkyl. In some embodiments, R is methyl. In some embodiments, R is ethyl. In some embodiments, a 2′-modification is (C2-O—CHR—C4), wherein R is optionally substituted C₁₋₆ aliphatic. In some embodiments, a 2′-modification is (C2-O—CHR—C4), wherein R is optionally substituted C₁₋₆ alkyl. In some embodiments, a 2′-modification is (C2-O—CHR—C4), wherein R is methyl. In some embodiments, a 2′-modification is (C2-O—CHR—C4), wherein R is ethyl. In some embodiments, a 2′-modification is (C2-O—(R)—CHR—C4), wherein R is optionally substituted C₁₋₆ aliphatic. In some embodiments, a 2′-modification is (C2-O—(R)—CHR—C4), wherein R is optionally substituted C₁₋₆ alkyl. In some embodiments, a 2′-modification is (C2-O—(R)—CHR—C4), wherein R is methyl. In some embodiments, a 2′-modification is (C2-O—(R)—CHR—C4), wherein R is ethyl. In some embodiments, a 2′-modification is (C2-O—(S)—CHR—C4), wherein R is optionally substituted C₁₋₆ aliphatic. In some embodiments, a 2′-modification is (C2-O—(S)—CHR—C4), wherein R is optionally substituted C₁₋₆ alkyl. In some embodiments, a 2′-modification is (C2-O—(S)—CHR—C4), wherein R is methyl. In some embodiments, a 2′-modification is (C2-O—(S)—CHR—C4), wherein R is ethyl. In some embodiments, a 2′-modification is C2-O—(R)—CH(CH₂CH₃)—C4. In some embodiments, a 2′-modification is C2-O—(S)—CH(CH₂CH₃)—C4. In some embodiments, a sugar is a natural DNA sugar. In some embodiments, a sugar is a natural RNA sugar. In some embodiments, a sugar is an optionally substituted natural DNA sugar. In some embodiments, a sugar is a natural DNA sugar optionally substituted at 2′. In some embodiments, a sugar is a natural DNA sugar substituted at 2′ (2′-modification). In some embodiments, a sugar is a natural DNA sugar modified at 2′ (2′-modification).

In some embodiments, a sugar is an optionally substituted ribose or deoxyribose. In some embodiments, a sugar is an optionally modified ribose or deoxyribose, wherein one or more hydroxyl groups of the ribose or deoxyribose moiety is optionally and independently replaced by halogen, R′, —N(R′)₂, —OR′, or —SR′, wherein each R′ is as described herein. In some embodiments, a sugar is an optionally substituted deoxyribose, wherein the 2′ position of the deoxyribose is optionally substituted. In some embodiments, a sugar is an optionally substituted deoxyribose, wherein the 2′ position of the deoxyribose is optionally substituted with halogen, R′, —N(R′)₂, —OR′, or —SR′, wherein each R′ is independently described in the present disclosure. In some embodiments, a sugar is an optionally substituted deoxyribose, wherein the 2′ position of the deoxyribose is optionally substituted with halogen. In some embodiments, a sugar is an optionally substituted deoxyribose, wherein the 2′ position of the deoxyribose is optionally substituted with one or more -F. In some embodiments, a sugar is an optionally substituted deoxyribose, wherein the 2′ position of the deoxyribose is optionally substituted with —OR′, wherein each R′ is independently described in the present disclosure. In some embodiments, a sugar is an optionally substituted deoxyribose, wherein the 2′ position of the deoxyribose is optionally substituted with —OR′, wherein each R′ is independently optionally substituted C₁-C₆ aliphatic. In some embodiments, a sugar is an optionally substituted deoxyribose, wherein the 2′ position of the deoxyribose is optionally substituted with —OR′, wherein each R′ is independently an optionally substituted C₁-C₆ alkyl. In some embodiments, a sugar is an optionally substituted deoxyribose, wherein the 2′ position of the deoxyribose is optionally substituted with —OMe. In some embodiments, a sugar is an optionally substituted deoxyribose, wherein the 2′ position of the deoxyribose is optionally substituted with —O-methoxyethyl.

In some embodiments, provided oligonucleotides comprise one or more modified sugars. In some embodiments, provided oligonucleotides comprise one or more modified sugars and one or more natural sugars.

Examples of bicyclic sugars include sugars of alpha-L-methyleneoxy (4′-CH₂—O-2′) LNA, beta-D-methyleneoxy (4′-CH₂—O-2′) LNA, ethyleneoxy (4′-(CH₂)2—O-2′) LNA, aminooxy (4′-CH₂—O—N(R)-2′) LNA, and oxyamino (4′-CH₂—N(R)-O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose.

In some embodiments, a bicyclic sugar may be further defined by isomeric configuration. For example, a sugar comprising a 4′-(CH₂)—O-2′ bridge may be in the alpha-L configuration or in the beta-D configuration. In some embodiments, a 4′ to 2′ bridge is a -L-4′-(CH₂)—O-2′, b-D-4′-CH₂—O-2′, 4′-(CH₂)2—O-2′, 4′-CH₂—O—N(R′)-2′, 4′-CH₂—N(R′)-O-2′, 4′-CH(R′)-O-2′, 4′-CH(CH₃)—O-2′, 4′-CH₂—S-2′, 4′-CH₂—N(R′)-2′, 4′-CH₂—CH(R′)-2′, 4′-CH₂—CH(CH₃)-2′, and 4′-(CH₂)₃-2′, wherein each R′ is as described in the present disclosure. In some embodiments, R′ is —H, a protecting group or optionally substituted C₁-C₁₂ alkyl. In some embodiments, R′ is —H or optionally substituted C₁-C₁₂ alkyl.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S-2′) BNA, methylene-amino (4′-CH₂—N(R)-2′) BNA, methyl carbocyclic (4′-CH₂—CH(CH₃)-2′) BNA, propylene carbocyclic (4′-(CH₂)3-2′) BNA, or vinyl BNA.

In some embodiments, a sugar modification is a modification described in U.S. Pat. No. 9,006,198. In some embodiments, a modified sugar is described in U.S. Pat. No. 9,006,198. In some embodiments, a sugar modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the sugar modifications and modified sugars of each of which are independently incorporated herein by reference.

In some embodiments a modified sugar is one described in U.S. Pat. Nos. 5,658,873, 5,118,800, 5,393,878, 5,514,785, 5,627,053, 7,034,133; 7,084,125, 7,399,845, 5,319,080, 5,591,722, 5,597,909, 5,466,786, 6,268,490, 6,525,191, 5,519,134, 5,576,427, 6,794,499, 6,998,484, 7,053,207, 4,981,957, 5,359,044, 6,770,748, 7,427,672, 5,446,137, 6,670,461, 7,569,686, 7,741,457, 8,022,193, 8,030,467, 8,278,425, 5,610,300, 5,646,265, 8,278,426, 5,567,811, 5,700,920, 8,278,283, 5,639,873, 5,670,633, 8,314,227, US 2008/0039618, US 2009/0012281, WO 2021/030778, WO 2020/154344, WO 2020/154343, WO 2020/154342, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof In some embodiments, a sugar is as in flexible nucleic acids or serinol nucleic acids. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc. In some embodiments, a sugar is a (R)-GNA sugar. In some embodiments, a sugar is a (S)-GNA sugar. In some embodiments, a nucleoside having a GNA sugar is utilized as N₋₁, No and/or N₁. In some embodiments, N₀ is a nucleoside having a GNA sugar. In some embodiments, a sugar is bicyclic sugar. In some embodiments, a sugar is a LNA sugar. In some embodiments, a sugar is an acyclic sugar. In some embodiments, a sugar is a UNA sugar. In some embodiments, a nucleoside having a UNA sugar is utilized as N₋₁, N₀ and/or N₁. In some embodiments, N₀ is a nucleoside having a UNA sugar. In some embodiments, a nucleoside is abasic. In some embodiments, an abasic sugar is utilized as N₋₁, N₀ and/or N₁. In some embodiments, N₀ is a nucleoside having an abasic sugar.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently described in the present disclosure; -O—(C₁-C₁₀ alkyl), —S—(C₁-C₁₀ alkyl), —NH-(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O-(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is -O(CH₂)ₙOCH₃, —O(CH₂)ₙNH₂, MOE, DMAOE, or DMAEOE, wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH-(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)2; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O-(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with -F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is not hydrogen and is as described in the present disclosure. In some embodiments, a modification is 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, one or more of the sugars of an oligonucleotide are modified. In some embodiments, a modified sugar comprises a 2′-modification. In some embodiments, each modified sugar independently comprises a 2′-modification. In some embodiments, a 2′-modification is 2′-OR. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR or 2′-F. In some embodiments, each sugar modification is independently 2′-OR or 2′-F, wherein R is optionally substituted C₁₋₆ alkyl. In some embodiments, each sugar modification is independently 2′-OR or 2′-F, wherein at least one is 2′-F. In some embodiments, each sugar modification is independently 2′-OR or 2′-F, wherein R is optionally substituted C₁₋₆ alkyl, and wherein at least one is 2′-OR. In some embodiments, each sugar modification is independently 2′-OR or 2′-F, wherein at least one is 2′-F, and at least one is 2′-OR. In some embodiments, each sugar modification is independently 2′-OR or 2′-F, wherein R is optionally substituted C₁₋₆ alkyl, and wherein at least one is 2′-F, and at least one is 2′-OR. In some embodiments, each sugar modification is independently 2′-OR. In some embodiments, each sugar modification is independently 2′-OR, wherein R is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

Modified sugars include cyclobutyl or cyclopentyl moieties in place of a pentofuranosyl sugar. Representative examples of such modified sugars include those described in U.S. Pat. Nos. 4,981,957, 5,118,800, 5,319,080, or 5,359,044. In some embodiments, the oxygen atom within the ribose ring is replaced by nitrogen, sulfur, selenium, or carbon. In some embodiments, —O— is replaced with —N(R′)—, —S—, —Se— or —C(R′)₂—. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

A non-limiting example of modified sugars is glycerol, which is part of glycerol nucleic acids (GNAs), e.g., as described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603.

A flexible nucleic acid (FNA) is based on a mixed acetal aminal of formyl glycerol, e.g., as described in Joyce G F et al., PNAS, 1987, 84, 4398-4402 and Heuberger BD and Switzer C, J. Am. Chem. Soc., 2008, 130, 412-413.

In some embodiments, an oligonucleotide, and/or a modified nucleoside thereof, comprises a sugar or modified sugar described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the sugars and modified sugars of each of which are independently incorporated herein by reference.

In some embodiments, one or more hydroxyl group in a sugar is optionally and independently replaced with halogen, R′—N(R′)₂, —OR′, or -SR′, wherein each R′ is independently described in the present disclosure.

In some embodiments, a modified nucleoside is any modified nucleoside described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the modified nucleosides of each of which are independently incorporated herein by reference.

In some embodiments, a sugar modification is 5′-vinyl (R or S), 5′-methyl (R or S), 2′—SH. 2′-F, 2′-OCH₃, 2′-OCH₂CH₃, 2′-OCH₂CH₂F or 2′-O(CH₂)₂OCH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)-(CH₂)₂—N(Rₘ)(Rₙ), wherein each allyl, amino and alkyl is optionally substituted, and each of R₁, Rₘ and Rₙ is independently R′ as described in the present disclosure. In some embodiments, each of R₁, Rₘ and Rₙ is independently —H or optionally substituted C₁-C₁₀ alkyl.

In some embodiments, bicyclic sugars comprise a bridge, e.g., -L^{b}-L^{b}-, -L-, etc. between two sugar carbons, e.g., between the 4′ and the 2′ ribosyl ring carbon atoms. In some embodiments, a bridge is 4′-(CH₂)—O-2′ (e.g., LNA sugars), 4′-(CH₂)—S-2′, 4′-(CH₂)2—O-2′ (e.g., ENA sugars), 4′-CH(R′)-O-2′ (e.g., 4′-CH(CH₃)—O-2′, 4′-CH(CH₂OCH₃)—O-2′, and examples in U.S. Pat. No. 7,399,845, etc.), 4′-CH(R′)₂—O-2′ (e.g., 4′-C(CH₃)(CH₃)—O-2′ and examples in WO 2009006478, etc.), 4′-CH₂—N(OR′)-2′ (e.g., 4′-CH₂-N(OCH₃)-2′, examples in WO 2008150729, etc.), 4′-CH₂—O—N(R′)-2′ (e.g., 4′-CH₂—O—N(CH₃)-2′, examples in US 20040171570, etc.), 4′-CH₂—N(R′)-O-2′ [e.g., wherein R is —H, C₁-C₁₂ alkyl, or a protecting group (e.g., see U.S. Pat. No. 7,427,672)], 4′-C(R′)₂—C(H)(R′)-2′ (e.g., 4′-CH₂—C(H)(CH₃)-2′, examples in Chattopadhyaya et al., J. Org. Chem., 2009, 74, 118-134, etc.), or 4′-C(R′)₂—C(=C(R′)₂)-2′ (e.g., 4′-CH₂—C(═CH₂)-2′, examples in WO 2008154401, etc.).

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars which are described in e.g., Braasch et al., Biochemistry, 2002, 41, 4503-4510; U.S. Pat. Nos. 5,698,685; 5,166,315; 5,185,444; 5,034,506; etc.).

As those skilled in the art will appreciate, modifications of sugars, nucleobases, internucleotidic linkages, etc. can and are often utilized in combination in oligonucleotides, e.g., see various oligonucleotides in Table 1.

In some embodiments, a nucleoside has a six-membered cyclohexenyl in place of the pentofuranosyl residue in naturally occurring nucleosides. Example cyclohexenyl nucleosides and preparation and uses thereof are described in, e.g., WO 2010036696; Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; WO 2006047842; WO 2001049687; etc.

Many monocyclic, bicyclic and tricyclic ring systems are suitable as sugar surrogates (modified sugars) and may be utilized in accordance with the present disclosure. See, e.g., Leumann, Christian J. Bioorg. & Med. Chem., 2002, 10, 841-854. Such ring systems can undergo various additional substitutions to further enhance their properties and/or activities.

In some embodiments, a 2′-modified sugar is a furanosyl sugar modified at the 2′ position. In some embodiments, a 2′-modification is halogen, -R′ (wherein R′ is not —H), —OR′ (wherein R′ is not —H), -SR′, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH₃, —O(CH₂)ₙF, —O(CH₂)ₙONH₂, —OCH₂C(═O)N(H)CH₃, and —O(CH₂)ₙON[(CH₂)ₙCH₃]2, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —C1, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification (e.g., see Baker et al., J. Biol. Chem., 1997, 272, 11944-12000). In some cases, a 2′-MOE modification has been reported as having improved binding affinity compared to unmodified sugars and to some other modified nucleosides, such as 2′- O-methyl, 2′-O-propyl, and 2′-O-aminopropyl. Oligonucleotides having the 2′-MOE modification have also been reported to be capable of inhibiting gene expression with promising features for in vivo use (see, e.g., Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926; etc.).

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)2SCH₃, —O(CH₂)20N(Rₘ)(Rₙ), or —OCH₂C(═O)N(Rₘ)(Rₙ), where each Rₘ and Rₙ is independently —H or optionally substituted C₁-C₁₀ alkyl.

Certain modified sugars, their preparation and uses are described in U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,670,633, 5,700,920, 5,792,847, 6,600,032 and WO 2005121371.

In some embodiments, a sugar is the sugar of N-methanocarba, LNA, cMOE BNA, cEt BNA, a-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage. In some embodiments, examples of internucleotidic linkages and/or sugars are described in Allerson et al. 2005 J. Med. Chem. 48: 901-4; BMCL 2011 21: 1122; BMCL 2011 21: 588; BMCL 2012 22: 296; Chattopadhyaya et al. 2007 J. Am. Chem. Soc. 129: 8362; Chem. Bio. Chem. 2013 14: 58; Curr. Prot. Nucl. Acids Chem. 2011 1.24.1; Egli et al. 2011 J. Am. Chem. Soc. 133: 16642; Hendrix et al. 1997 Chem. Eur. J. 3: 110; Hyrup et al. 1996 Bioorg. Med. Chem. 4: 5; Imanishi 1997 Tet. Lett. 38: 8735; J. Am. Chem. Soc. 1994, 116, 3143; J. Med. Chem. 2009 52: 10; J. Org. Chem. 2010 75: 1589; Jepsen et al. 2004 Oligo. 14: 130-146; Jones et al. J. Org. Chem. 1993, 58, 2983; Jung et al. 2014 ACIEE 53: 9893; Kodama et al. 2014 AGDS; Koizumi 2003 BMC 11: 2211; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Lima et al. 2012 Cell 150: 883-894; Mesmaeker et al. Angew. Chem., Int. Ed. Engl. 1994, 33, 226; Migawa et al. 2013 Org. Lett. 15: 4316; Mol. Ther. Nucl. Acids 2012 1: e47; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Murray et al. 2012 Nucl. Acids Res. 40: 6135; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Obika et al. 2008 J. Am. Chem. Soc. 130: 4886; Obika et al. 2011 Org. Lett. 13: 6050; Oestergaard et al. 2014 JOC 79: 8877; Pallan et al. 2012 Biochem. 51: 7; Pallan et al. 2012 Chem. Comm. 48: 8195-8197; Petersen et al. 2003 TRENDS Biotech. 21: 74-81; Prakash et al. 2010 J. Med. Chem. 53: 1636; Prakash et al. 2015 Nucl. Acids Res. 43: 2993-3011; Prakash et al. 2016 Bioorg. Med. Chem. Lett. 26: 2817-2820; Rajwanshi et al. 1999 Chem. Commun. 1395-1396; Schultz et al. 1996 Nucleic Acids Res. 24: 2966; Seth et al. 2008 Nucl. Acid Sym. Ser. 52: 553; Seth et al. 2009 J. Med. Chem. 52: 10-13; Seth et al. 2010 J. Am. Chem. Soc. 132: 14942; Seth et al. 2010 J. Med. Chem. 53: 8309-8318; Seth et al. 2010 J. Org. Chem. 75: 1569-1581; Seth et al. 2011 BMCL 21: 4690; Seth et al. 2012 Bioo. Med. Chem. Lett. 22: 296-299; Seth et al. 2012 Mol. Ther-Nuc. Acids. 1, e47; Seth et al., Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Starrup et al. 2010 Nucl. Acids Res. 38: 7100; Swayze et al. 2007 Nucl. Acids Res. 35: 687; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114,4006; WO 2007090071; WO 2016079181; U.S. Pat. Nos. 6,326,199; 6,066,500; or U.S. Pat. No. 6,440,739.

In some embodiments, an oligonucleotide or a portion thereof (e.g., a domain, a subdomain, etc.) comprise a high level of 2′-F modified sugars, e.g., about 10%-100% (e.g., about 20%, 25%, 30%, 35%, 40%, 45%, 50%, 55%, 60%, 65%, 70%, 75%, 80%, 85%, 90%, 95%, or more, or about 100%) of sugars in an oligonucleotide or a portion thereof (e.g., a domain, a subdomain, etc.) comprises 2′-F. In some embodiments, about 50% or more of sugars in an oligonucleotide or a portion thereof comprises 2′-F. In some embodiments, about 60% or more of sugars in an oligonucleotide or a portion thereof comprises 2′-F. In some embodiments, about 70% or more of sugars in an oligonucleotide or a portion thereof comprises 2′-F. In some embodiments, about 80% or more of sugars in an oligonucleotide or a portion thereof comprises 2′-F. In some embodiments, about 90% or more of sugars in an oligonucleotide or a portion thereof comprises 2′-F. In some embodiments, an oligonucleotide or a portion thereof also comprises one or more sugars comprising no 2′-F (e.g., sugars comprising no modifications and/or sugars comprising other modifications).

In some embodiments, no more than about 1%-95% (e.g., no more than about 1%, 5%, 10%, 15%, 20%, 25%, 30%, 35%, 40%, 45%, 50%, 55%, 60%, 65, 70%, 75%, 80%, 85%, 90%, 95%, etc.) of sugars in an oligonucleotide or a portion thereof (e.g., a domain, a subdomain, etc.) comprises 2′-MOE. In some embodiments, no more than about 50% of sugars in an oligonucleotide or a portion thereof comprises 2′-MOE. In some embodiments, no sugars in an oligonucleotide or a portion thereof comprises 2′-MOE. In some embodiments, no more than 1, 2, 3, 4, or 5 sugars in an oligonucleotide or a portion thereof comprises 2′-MOE.

Various additional sugars useful for preparing oligonucleotides or analogs thereof are known in the art and may be utilized in accordance with the present disclosure.

### Internucleotidic Linkages

Among other things, the present disclosure provides various internucleotidic linkages, including various modified internucleotidic linkages, that may be utilized together with other structural elements, e.g., various sugars as described herein, to provide oligonucleotides and compositions thereof.

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, provided oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof Modified internucleotidic linkages, depending on their structures, may also be in their salt forms. For example, as appreciated by those skilled in the art, phosphorothioate internucleotidic linkages which have the structure of —OP(O)(SH)O- may be in various salt forms, e.g., at physiological pH (about 7.4) with the anion being —OP(O)(S⁻)O—.

Oligonucleotides may comprise one or more types of internucleotidic linkages. In some embodiments, an oligonucleotide comprises one or more PS linkages. In some embodiments, an oligonucleotide comprises one or more PO linkages. In some embodiments, an oligonucleotide comprises one or more PN linkages. In some embodiments, an oligonucleotide comprises one or more PS and one or more PO linkages. In some embodiments, an oligonucleotide comprises one or more PS and one or more PN linkages. In some embodiments, an oligonucleotide comprises one or more PS, one or more PN and one or more PO linkages. PO linkages may be utilized at various positions, and/or in various patterns, including those described herein for natural phosphate linkages. In some embodiments, a modified internucleotidic linkage is PS linkage. In some embodiments, a chiral internucleotidic linkage is a PS linkage. In some embodiments, a PS linkage is a phosphorothioate linkage. PS linkages may be utilized at various positions, stereochemistry and/or in various patterns, including those described herein for phosphorothioate internucleotidic linkages. In some embodiments, a modified internucleotidic linkage is a PN linkage. In some embodiments, a modified internucleotidic linkage is a chiral PN linkage. PN linkages may be utilized at various positions, stereochemistry and/or in various patterns, including those described herein for non-negatively charged internucleotidic linkages, neutral internucleotidic linkages phosphoryl guanidine internucleotidic linkages and/or n001. In some embodiments, a PN linkage is a non-negatively charged internucleotidic linkage. In some embodiments, a PN linkage is a neutral internucleotidic linkage. In some embodiments, a PN linkage is a phosphoryl guanidine linkage. In some embodiments, a PN linkage is n001. In some embodiments, a non-negatively charged internucleotidic linkage is a PN linkage. In some embodiments, a neutral internucleotidic linkage is a PN internucleotidic linkage.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, an internucleotidic linkage comprises a P-modification, wherein a P-modification is a modification at a linkage phosphorus. In some embodiments, a modified internucleotidic linkage is a moiety which does not comprise a phosphorus but serves to link two sugars or two moieties that each independently comprises a nucleobase, e.g., as in peptide nucleic acid (PNA).

In some embodiments, an oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the internucleotidic linkages (e.g., those of Formula 1, 1-a, 1-b, I-c, etc.) of each of which are independently incorporated herein by reference. In some embodiments, a modified internucleotidic linkage is a chiral internucleotidic linkage. In some embodiments, a modified internucleotidic linkage is a phosphorothioate internucleotidic linkage.

In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage has the structure of Formula I-n-1, I-n-2, I-n-3, I-n-4, II, II-a-1, II-a-2, II-b-1, II-b-2, 1I-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the non-negatively charged internucleotidic linkages (e.g., those of Formula I-n-1, I-n-2. I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In some embodiments, a non-negatively charged internucleotidic linkage can improve the delivery and/or activities (e.g., adenosine editing activity).

In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage has the structure of

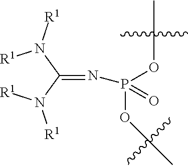

and is optionally chirally controlled, wherein R¹ is -L-R′, wherein L is L^{B} as described herein, and R′ is as described herein. In some embodiments, each R¹ is independently R′. In some embodiments, each R′ is independently R. In some embodiments, two R¹ are R and are taken together to form a ring as described herein. In some embodiments, two R¹ on two different nitrogen atoms are R and are taken together to form a ring as described herein. In some embodiments, R¹ is independently optionally substituted C₁₋₆ aliphatic as described herein. In some embodiments, R¹ is methyl. In some embodiments, two R′ on the same nitrogen atom are R and are taken together to form a ring as described herein. In some embodiments, a modified internucleotidic linkage has the structure of

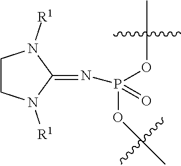

and is optionally chirally controlled. In some embodiments,

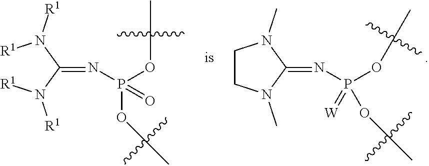

In some embodiments, a modified internucleotidic linkage comprises an optionally substituted cyclic guanidine moiety and has the structure of:

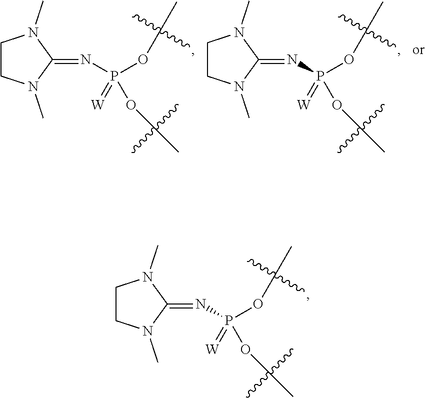

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

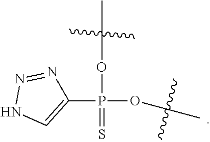 [SMILES: *OP(=S)(O*)c1c[nH]nn1]

In some embodiments, an internucleotidic linkage comprising a triazole moiety has the structure of

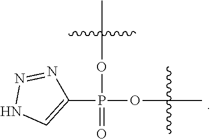 [SMILES: *OP(=O)(O*)c1c[nH]nn1]

In some embodiments, an internucleotidic linkage comprising a triazole moiety has the formula of

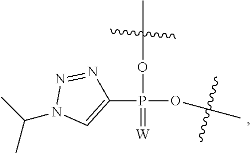

where W is O or S. In some embodiments, an internucleotidic linkage comprising an alkyne moiety (e.g., an optionally substituted alkynyl group) has the formula of

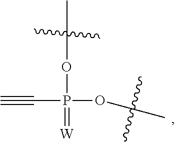

wherein W is O or S. In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

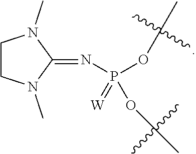

In some embodiments, a non-negatively charged internucleotidic linkage, or a neutral internucleotidic linkage, is or comprising a structure selected from

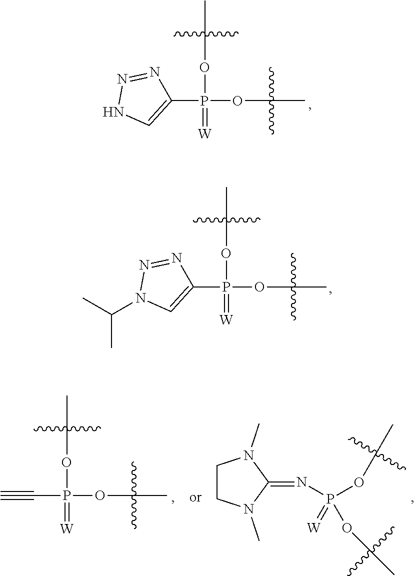

wherein W is O or S.

In some embodiments, an internucleotidic linkage comprises a Tmg group

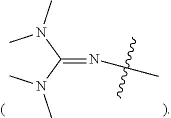 [SMILES: *N=C(N(C)C)N(C)C]

In some embodiments, an internucleotidic linkage comprises a Tmg group and has the structure of

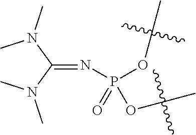 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, etc., or a salt form thereof. In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted triazolyl group. In some embodiments, a non-negatively charged internucleotidic linkage comprises an unsubstituted triazolyl group, e.g.,

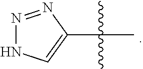 [SMILES: *c1c[nH]nn1]

In some embodiments, a non-negatively charged internucleotidic linkage comprises a substituted triazolyl group, e.g.,

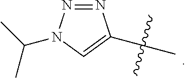 [SMILES: *c1cn(C(C)C)nn1]

In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

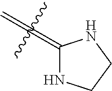 [SMILES: *=C1NCCN1]

group. In some embodiments, a non-negatively charged internucleotidic linkage comprises an substituted

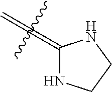 [SMILES: *=C1NCCN1]

group. In some embodiments, a non-negatively charged internucleotidic linkage comprises a

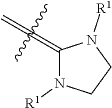

group, wherein each R¹ is independently -L-R. In some embodiments, each R¹ is independently optionally substituted C₁₋₆ alkyl. In some embodiments, each R¹ is independently methyl.

In some embodiments, a modified internucleotidic linkage, e.g., a non-negatively charged internucleotidic linkage, comprises a triazole or alkyne moiety, each of which is optionally substituted. In some embodiments, a modified internucleotidic linkage comprises a triazole moiety. In some embodiments, a modified internucleotidic linkage comprises a unsubstituted triazole moiety. In some embodiments, a modified internucleotidic linkage comprises a substituted triazole moiety. In some embodiments, a modified internucleotidic linkage comprises an alkyl moiety. In some embodiments, a modified internucleotidic linkage comprises an optionally substituted alkynyl group. In some embodiments, a modified internucleotidic linkage comprises an unsubstituted alkynyl group. In some embodiments, a modified internucleotidic linkage comprises a substituted alkynyl group. In some embodiments, an alkynyl group is directly bonded to a linkage phosphorus.

In some embodiments, an oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, an oligonucleotide comprises at least one natural phosphate linkage and at least one phosphorothioate. In some embodiments, an oligonucleotide comprises at least one non-negatively charged internucleotidic linkage. In some embodiments, an oligonucleotide comprises at least one natural phosphate linkage and at least one non-negatively charged internucleotidic linkage. In some embodiments, an oligonucleotide comprises at least one phosphorothioate internucleotidic linkage and at least one non-negatively charged internucleotidic linkage. In some embodiments, an oligonucleotide comprises at least one phosphorothioate internucleotidic linkage, at least one natural phosphate linkage, and at least one non-negatively charged internucleotidic linkage. In some embodiments, oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In some embodiments, oligonucleotides comprise no more than a certain number of non-negatively charged internucleotidic linkages, e.g., no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18. no more than 19, no more than 20, no more than 21, no more than 22, no more than 23, no more than 24, no more than 25, no more than 26, no more than 27, no more than 28, no more than 29, or no more than 30 non-negatively charged internucleotidic linkages. In some embodiments, oligonucleotides comprise no non-negatively charged internucleotidic linkages. In some embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In some embodiments, a pH is about pH 7.4. In some embodiments, a pH is about 4-9. In some embodiments, the percentage is less than 10%. In some embodiments, the percentage is less than 5%. In some embodiments, the percentage is less than 1%. In some embodiments, an internucleotidic linkage is a non-negatively charged internucleotidic linkage in that the neutral form of the internucleotidic linkage has no pKa that is no more than about 1, 2, 3, 4, 5, 6, or 7 in water. In some embodiments, no pKa is 7 or less. In some embodiments, no pKa is 6 or less. In some embodiments, no pKa is 5 or less. In some embodiments, no pKa is 4 or less. In some embodiments, no pKa is 3 or less. In some embodiments, no pKa is 2 or less. In some embodiments, no pKa is 1 or less. In some embodiments, pKa of the neutral form of an internucleotidic linkage can be represented by pKa of the neutral form of a compound having the structure of CH₃— the internucleotidic linkage —CH₃. For example, pKa of the neutral form of an internucleotidic linkage having the structure of Formula I may be represented by the pKa of the neutral form of a compound having the structure of

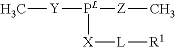

(wherein each of X, Y, Z is independently —O—, —S—, —N(R′)—; L is L^{B}, and R¹ is -L-R′), pKa of

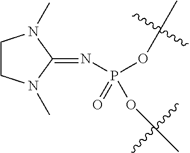 [SMILES: *OP(=O)(N=C1N(C)CCN1C)O*]

can be represented by pKa

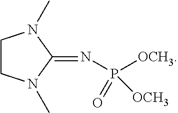

In some embodiments, a non-negatively charged internucleotidic linkage is a neutral internucleotidic linkage. In some embodiments, a non-negatively charged internucleotidic linkage is a positively-charged internucleotidic linkage. In some embodiments, a non-negatively charged internucleotidic linkage comprises a guanidine moiety. In some embodiments, a non-negatively charged internucleotidic linkage comprises a heteroaryl base moiety. In some embodiments, a non-negatively charged internucleotidic linkage comprises a triazole moiety. In some embodiments, a non-negatively charged internucleotidic linkage comprises an alkynyl moiety.

In some embodiments, a neutral or non-negatively charged internucleotidic linkage has the structure of any neutral or non-negatively charged internucleotidic linkage described in any of. U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2017/18/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, each neutral or non-negatively charged internucleotidic linkage of each of which is hereby incorporated by reference.

In some embodiments, each R′ is independently optionally substituted C₁₋₆ aliphatic. In some embodiments, each R′ is independently optionally substituted C₁₋₆ alkyl. In some embodiments, each R′ is independently —CH₃. In some embodiments, each R^{s} is —H.

In some embodiments, a non-negatively charged internucleotidic linkage has the structure of

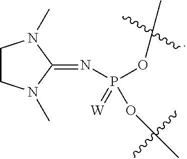

In some embodiments, a non-negatively charged internucleotidic linkage has the structure of

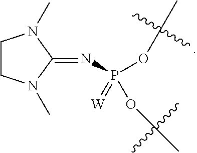

In some embodiments, a non-negatively charged internucleotidic linkage has the structure of

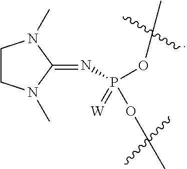

In some embodiments, a non-negatively charged internucleotidic linkage has the structure of

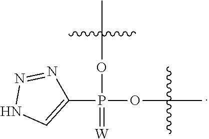

In some embodiments, a non-negatively charged internucleotidic linkage has the structure of

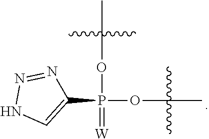

In some embodiments, a non-negatively charged internucleotidic linkage has the structure of

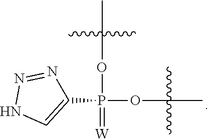

In some embodiments, a non-negatively charged internucleotidic linkage has the structure of

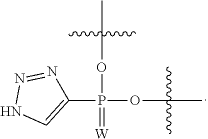

In some embodiments, a non-negatively charged internucleotidic linkage has the structure of

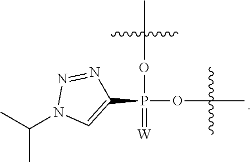

In some embodiments, a non-negatively charged internucleotidic linkage has the structure of

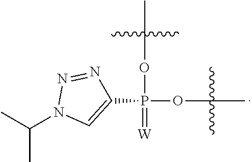

In some embodiments, a non-negatively charged internucleotidic linkage has the structure of

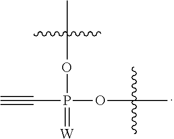

In some embodiments, a non-negatively charged internucleotidic linkage has the structure of

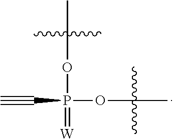

In some embodiments, a non-negatively charged internucleotidic linkage has the structure of

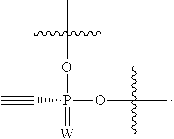

In some embodiments, W is O. In some embodiments, W is S. In some embodiments, a neutral internucleotidic linkage is a non-negatively charged internucleotidic linkage described above.

In some embodiments, provided oligonucleotides comprise I or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, which are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or salt forms thereof, each of which are independently incorporated herein by reference.

In some embodiments, an oligonucleotide comprises a neutral internucleotidic linkage and a chirally controlled internucleotidic linkage. In some embodiments, an oligonucleotide comprises a neutral internucleotidic linkage and a chirally controlled internucleotidic linkage which is not the neutral internucleotidic linkage. In some embodiments, an oligonucleotide comprises a neutral internucleotidic linkage and a chirally controlled phosphorothioate internucleotidic linkage. In some embodiments, the present disclosure provides an oligonucleotide comprising one or more non-negatively charged internucleotidic linkages and one or more phosphorothioate internucleotidic linkages, wherein each phosphorothioate internucleotidic linkage in the oligonucleotide is independently a chirally controlled internucleotidic linkage. In some embodiments, the present disclosure provides an oligonucleotide comprising one or more neutral internucleotidic linkages and one or more phosphorothioate internucleotidic linkage, wherein each phosphorothioate internucleotidic linkage in the oligonucleotide is independently a chirally controlled internucleotidic linkage. In some embodiments, an oligonucleotide comprises at least 5, 6, 7, 8, 9, 10, 11, 12, 13, 14, 15, 16, 17, 18, 19, 20 or more chirally controlled phosphorothioate internucleotidic linkages. In some embodiments, non-negatively charged internucleotidic linkage is chirally controlled. In some embodiments, non-negatively charged internucleotidic linkage is not chirally controlled. In some embodiments, a neutral internucleotidic linkage is chirally controlled. In some embodiments, a neutral internucleotidic linkage is not chirally controlled. In some embodiments, an oligonucleotide comprises one or more (e.g., 1, 2, 3, 4, 5, 6, 7, 8, 9, or 10) chirally controlled and one or more (e.g., 1, 2, 3, 4, 5, 6, 7, 8, 9, or 10) non-chirally controlled chiral internucleotidic linkages. In some embodiments, an oligonucleotide comprises one or more (e.g., 1, 2, 3, 4, 5, 6, 7, 8, 9, or 10) chirally controlled and one or more (e.g., 1, 2, 3, 4, 5, 6, 7, 8, 9, or 10) non-chirally controlled non-negatively charged internucleotidic linkages (in some embodiments, each of which is independently n001). In some embodiments, a neutral internucleotidic linkage is chirally controlled. In some embodiments, a neutral internucleotidic linkage is not chirally controlled. In some embodiments, an oligonucleotide comprises one or more (e.g., 1, 2, 3, 4, 5, 6, 7, 8, 9, or 10) chirally controlled and one or more (e.g., 1, 2, 3, 4, 5, 6, 7, 8, 9, or 10) non-chirally controlled neutral internucleotidic linkages (in some embodiments, each of which is independently n001).

Without wishing to be bound by any particular theory, the present disclosure notes that a neutral internucleotidic linkage can be more hydrophobic than a phosphorothioate internucleotidic linkage, which can be more hydrophobic than a natural phosphate linkage. Typically, unlike a phosphorothioate internucleotidic linkage or a natural phosphate linkage, a neutral internucleotidic linkage bears less charge. Without wishing to be bound by any particular theory, the present disclosure notes that incorporation of one or more neutral internucleotidic linkages into an oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between an oligonucleotide and its target nucleic acid.

Without wishing to be bound by any particular theory, the present disclosure notes that incorporation of one or more non-negatively charged internucleotidic linkages, e.g., neutral internucleotidic linkages, into an oligonucleotide may be able to increase the oligonucleotide's ability to mediate a function such as target adenosine editing.

As appreciated by those skilled in the art, internucleotidic linkages such as natural phosphate linkages and those of Formula 1, 1-a, 1-b, 1-c, 1-n-1. 1-n-2, 1-n-3, 1-n-4, 11, 11-a-1, 11-a-2, 11-b-1, 11-b-2, 11-c-1, 11-c-2, 11-d-1, 11-d-2, or salt forms thereof typically connect two nucleosides (which can either be natural or modified) as described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the Formula I, I-a, I-b, I-c, I-n-1, I-n-2, I-n-3, I-n-4, II, II-a-1, II-a-2, II-b-1, II-b-2, 1I-c-1, II-c-2, II-d-1, II-d-2, or salt forms thereof, each of which are independently incorporated herein by reference. A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms (e.g., Y and Z in various formulae) with one optionally modified ribose or deoxyribose at its 5′ carbon, and the other optionally modified ribose or deoxyribose at its 3′ carbon. In some embodiments, each nucleoside units connected by an internucleotidic linkage independently comprises a nucleobase which is independently an optionally substituted A, T, C, G, or U, or a substituted tautomer of A, T, C, G or U, or a nucleobase comprising an optionally substituted heterocyclyl and/or a heteroaryl ring having at least one nitrogen atom.

In some embodiments, a linkage has the structure of or comprises —Y—P^{L}(_X—R^{L})-Z-, or a salt form thereof, wherein:
P^{L} is P, P(=W), P->B(-L^{L}-R^{L})₃, or P^{N};
W is O, N(-L^{L}-R^{L}), S or Se;
P^{N} is P=N-C(-L^{L}-R′)(=L^{N}-R′) or P=N-L^{L}-R^{L};
L^{N} is =N-L^{L1}-, ═CH-L^{L1}- wherein CH is optionally substituted, or ═N⁺(R′)(Q⁻)-L^{L1}-;
Q⁻ is an anion;
each of X, Y and Z is independently —O—, —S—, -L^{L}-N(-L^{L}-R^{L})-L^{L}-, -L^{L}-N═C(-L^{L}-R^{L})-L^{L} or L^{L};
each R^{L} is independently -L^{L}-N(R′)₂, -L^{L}-R′, —N═C(-L^{L}-R′)₂, -L^{L}-N(R′)C(NR′)N(R′)₂, -L^{L}-N(R′)C(O)N(R′)₂, a carbohydrate, or one or more additional chemical moieties optionally connected through a linker;
each of L^{L1} and L^{L} is independently L;
—Cy^{IL}— is —Cy—;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, —Cy—, —O—, —S—, —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]-, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, and -[C(R′)₂C(R′)₂O]n-, wherein n is 1-50, and one or more nitrogen or carbon atoms are optionally and independently replaced with Cy^{L};
each —Cy— is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)N(R)₂, —C(O)OR, or -S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆_3₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an internucleotidic linkage has the structure of —O-P^{L}(—X—R^{L})—O—, wherein each variable is independently as described herein. In some embodiments, an internucleotidic linkage has the structure of —O—P(═W)(—X—R^{L})—O—, wherein each variable is independently as described herein. In some embodiments, an internucleotidic linkage has the structure of —O—P(═W)[—N(-L^{L}-R^{L})-R^{L}]-O—, wherein each variable is independently as described herein. In some embodiments, an internucleotidic linkage has the structure of —O—P(═W)(-NH-L^{L}-R^{L})—O—, wherein each variable is independently as described herein. In some embodiments, an internucleotidic linkage has the structure of-O—P(═W)[—N(R′)₂]—O—, wherein each variable is independently as described herein. In some embodiments, an internucleotidic linkage has the structure of —O—P(═W)(-NHR′)—O—, wherein each variable is independently as described herein. In some embodiments, an internucleotidic linkage has the structure of —O—P(═W)(-NHSO₂R)—O—, wherein each variable is independently as described herein. In some embodiments, R is methyl. In some embodiments, an internucleotidic linkage is —O—P(═O)(-NHSO₂CH₃)—O—. In some embodiments, an internucleotidic linkage has the structure of —O—P(═W)└—N═C(-L^{L}-R′)₂┘-O—, wherein each variable is independently as described herein. In some embodiments, an internucleotidic linkage has the structure of -O—P(═W)[—N═C[N(R′)₂]₂]—O—, wherein each variable is independently as described herein. In some embodiments, an internucleotidic linkage has the structure of —OP(=W)(-N═C(R″)₂)—O—, wherein each variable is independently as described herein. In some embodiments, an internucleotidic linkage has the structure of —OP(=W)(-N(R″)₂)-O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage.

In some embodiments, an internucleotidic linkage has the structure of -P^{L}(—X—R^{L})-Z-, wherein each variable is independently as described herein. In some embodiments, an internucleotidic linkage has the structure of -P^{L}(—X—R^{L})—O- , wherein each variable is independently as described herein. In some embodiments, an internucleotidic linkage has the structure of -P(=W)(—X—R^{L})—O—, wherein each variable is independently as described herein. In some embodiments, an internucleotidic linkage has the structure of -P(=W)[—N(-L^{L}-R^{L})-R^{L}]-O—, wherein each variable is independently as described herein. In some embodiments, an internucleotidic linkage has the structure of -P(=W)(-NH-L^{L}-R^{L})—O—, wherein each variable is independently as described herein. In some embodiments, an internucleotidic linkage has the structure of -P(=W)[—N(R′)₂]—O—, wherein each variable is independently as described herein. In some embodiments, an internucleotidic linkage has the structure of -P(=W)(-NHR′)—O—, wherein each variable is independently as described herein. In some embodiments, an internucleotidic linkage has the structure of -P(=W)(-NHSO₂R)-O—, wherein each variable is independently as described herein. In some embodiments, R is methyl. In some embodiments, an internucleotidic linkage is —P(═O)(-NHSO₂CH₃)—O—. In some embodiments, an internucleotidic linkage has the structure of -P(=W)[—N═C(-L^{L}-R′)₂]—O—, wherein each variable is independently as described herein. In some embodiments, an internucleotidic linkage has the structure of -P(=W)[—N═C[N(R′)₂]₂]—O—, wherein each variable is independently as described herein. In some embodiments, an internucleotidic linkage has the structure of -P(=W)(-N═C(R″)₂)—O—, wherein each variable is independently as described herein. In some embodiments, an internucleotidic linkage has the structure of -P(=W)(-N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage. In some embodiments, P of such an internucleotidic linkage is bonded to N of a sugar.

In some embodiments, a linkage is a phosphoryl guanidine internucleotidic linkage. In some embodiments, a linkage is a thio-phosphoryl guanidine internucleotidic linkage.

In some embodiments, one or more methylene units are optionally and independently replaced with a moiety as described herein. In some embodiments, L or L^{L} is or comprises —SO₂—. In some embodiments, L or L^{L} is or comprises —SO₂N(R′)-. In some embodiments, L or L^{L} is or comprises —C(O)—. In some embodiments, L or L^{L} is or comprises —C(O)O—. In some embodiments, L or L^{L} is or comprises —C(O)N(R′)-. In some embodiments, L or L^{L} is or comprises —P(═W)(R′)-. In some embodiments, L or L^{L}is or comprises —P(═O)(R′)-. In some embodiments, L or L^{L} is or comprises —P(═S)(R′)-. In some embodiments, L or L^{L} is or comprises —P(R′)-. In some embodiments, L or L^{L} is or comprises —P(═W)(OR′)-. In some embodiments, L or L^{L} is or comprises —P(═O)(OR′)-. In some embodiments, L or L^{L} is or comprises —P(═S)(OR′)-. In some embodiments, L or L^{L} is or comprises -P(OR′)-.

In some embodiments, —X—R^{L} is —N(R′)SO₂R^{L}. In some embodiments, —X—R^{L} is —N(R′)C(O)R^{L}. In some embodiments, —X—R^{L} is —N(R′)P(=O)(R′)R^{L}.

In some embodiments, a linkage, e.g., a non-negatively charged internucleotidic linkage or neutral internucleotidic linkage, has the structure of or comprises —P(═W)(-N═C(R″)₂)-, —P(═W)(-N(R′)SO₂R″)-, —P(═W)(-N(R′)C(O)R″)-, —P(═W)(-N(R″)₂)-, —P(═W)(-N(R′)P(O)(R″)₂)-, —OP(═W)(-N═C(R″)₂)O—, —OP(═W)(-N(R′)SO₂R″)O—, —OP(═W)(-N(R′)C(O)R″)O—, —OP(═W)(-N(R″)₂)O—, —OP(═W)(-N(R′)P(O)(R″)₂)O—, —P(═W)(-N═C(R″)₂)O—, —P(═W)(-N(R′)SO₂R″)O—, —P(═W)(-N(R′)C(O)R″)O—, —P(═W)(-N(R″)₂)O—, or —P(═W)(-N(R′)P(O)(R″)₂)O—, or a salt form thereof, wherein:
W is O or S;
each R″ is independently R′, —OR′, —P(═W)(R′)₂, or —N(R′)₂;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, W is O. In some embodiments, an internucleotidic linkage has the structure of -P(═O)(-N═C(R″)₂)-, —P(═O)(-N(R′)SO₂R″)-, —P(═O)(-N(R′)C(O)R″)-, —P(═O)(-N(R″)₂)-, —P(═O)(-N(R′)P(O)(R″)2)-, —OP(═O)(-N═C(R″)₂)O—, —OP(═O)(-N(R′)SO₂R″)O—, —OP(═O)(-N(R′)C(O)R″)O—, —OP(═O)(-N(R″)₂)O—, —OP(═O)(-N(R′)P(O)(R″)₂)O—, —P(═O)(-N═C(R″)₂)O—, —P(═O)(-N(R′)SO₂R″)O—, —P(═O)(-N(R′)C(O)R″)O—, —P(═O)(-N(R″)₂)O—, or —P(═O)(-N(R′)P(O)(R″)₂)O—, or a salt form thereof. In some embodiments, an internucleotidic linkage has the structure of -P(═O)(-N═C(R″)₂)- -P(═O)(-N(R″)₂)—, —OP(═O)(-N═C(R″)₂)—O—, —OP(═O)(-N(R″)₂)—O—, —P(═O)(-N═C(R″)₂)—O— or —P(═O)(-N(R″)₂)-O- or a salt form thereof. In some embodiments, an internucleotidic linkage has the structure of —OP(═O)(-N═C(R″)₂)—O— or —OP(═O)(-N(R″)₂)-O—, or a salt form thereof In some embodiments, an internucleotidic linkage has the structure of —OP(═O)(-N═C(R″)₂)-O—, or a salt form thereof. In some embodiments, an internucleotidic linkage has the structure of —OP(═O)(-N(R″)₂)-O—, or a salt form thereof In some embodiments, an internucleotidic linkage has the structure of —OP(═O)(-N(R′)SO₂R″)O—, or a salt form thereof. In some embodiments, an internucleotidic linkage has the structure of —OP(═O)(-N(R′)C(O)R″)O—, or a salt form thereof In some embodiments, an internucleotidic linkage has the structure of -OP(═O)(-N(R′)P(O)(R″)₂)O—, or a salt form thereof. In some embodiments, a internucleotidic linkage is n001.

In some embodiments, W is S. In some embodiments, an internucleotidic linkage has the structure of -P(═S)(-N═C(R″)₂)-, —P(═S)(-N(R′)SO₂R″)-, —P(═S)(-N(R′)C(O)R″)-, —P(═S)(-N(R″)₂)-, —P(═S)(-N(R′)P(O)(R″)2)-, —OP(═S)(-N═C(R″)2)O—, —OP(═S)(-N(R′)SO₂R″)O—, —OP(═S)(-N(R′)C(O)R″)O—, —OP(═S)(-N(R″)₂)O—, —OP(═S)(-N(R′)P(O)(R″)2)O—, —P(═S)(-N═C(R″)₂)O—, —P(═S)(-N(R′)SO₂R″)O—, —P(═S)(-N(R′)C(O)R″)O—, —P(═S)(-N(R″)₂)O—, or P(═S)(-N(R′)P(O)(R″)2)O, or a salt form thereof In some embodiments, an internucleotidic linkage has the structure of -P(═S)(-N═C(R″)₂)- -P(═S)(-N(R″)₂)-, —OP(═S)(-N═C(R′)₂)-O—, —OP(═S)(-N(R″)₂)-O—, —P(═S)(-N═C(R″)₂)-O- or —P(═S)(-N(R″)₂)-O- or a salt form thereof. In some embodiments, an internucleotidic linkage has the structure of —OP(═S)(-N═C(R″)₂)-O- or —OP(═S)(-N(R″)₂)-O—, or a salt form thereof. In some embodiments, an internucleotidic linkage has the structure of —OP(═S)(-N═C(R″)₂)-O—, or a salt form thereof In some embodiments, an internucleotidic linkage has the structure of —OP(═S)(-N(R″)₂)-O—, or a salt form thereof. In some embodiments, an internucleotidic linkage has the structure of —OP(═S)(-N(R′)SO₂R″)O—, or a salt form thereof In some embodiments, an internucleotidic linkage has the structure of —OP(═S)(-N(R′)C(O)R″)O—, or a salt form thereof. In some embodiments, an internucleotidic linkage has the structure of —OP(═S)(-N(R′)P(O)(R″)₂)O—, or a salt form thereof In some embodiments, a internucleotidic linkage is *n001.

In some embodiments, an internucleotidic linkage has the structure of -P(═O)(-N(R′)SO₂R″)-, wherein R″ is as described herein. In some embodiments, an internucleotidic linkage has the structure of -P(═S)(-N(R′)SO₂R″)-, wherein R″ is as described herein. In some embodiments, an internucleotidic linkage has the structure of -P(═O)(-N(R′)SO₂R″)O—, wherein R″ is as described herein. In some embodiments, an internucleotidic linkage has the structure of -P(═S)(-N(R′)SO₂R″)O—, wherein R″ is as described herein. In some embodiments, an internucleotidic linkage has the structure of —OP(═O)(-N(R′)SO₂R″)O—, wherein R″ is as described herein. In some embodiments, an internucleotidic linkage has the structure of -OP(═S)(-N(R′)SO₂R″)O—, wherein R″ is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in -SO₂R″, is R′ as described herein. In some embodiments, an internucleotidic linkage has the structure of -P(═O)(-NHSO₂R″)-, wherein R″ is as described herein. In some embodiments, an internucleotidic linkage has the structure of -P(═S)(-NHSO₂R″)-, wherein R″ is as described herein. In some embodiments, an internucleotidic linkage has the structure of -P(═O)(-NHSO₂R″)O—, wherein R″ is as described herein. In some embodiments, an internucleotidic linkage has the structure of -P(═S)(-NHSO₂R″)O—, wherein R″ is as described herein. In some embodiments, an internucleotidic linkage has the structure of —OP(═O)(-NHSO₂R″)O—, wherein R″ is as described herein. In some embodiments, an internucleotidic linkage has the structure of —OP(═S)(-NHSO₂R″)O—, wherein R″ is as described herein. In some embodiments, —X—R^{L} is —N(R′)SO₂R^{L}, wherein each of R′ and R^{L} is independently as described herein. In some embodiments, R^{L} is R″. In some embodiments, R^{L} is R′. In some embodiments, —X—R^{L} is —N(R′)SO₂R″, wherein R′ is as described herein. In some embodiments, —X—R^{L} is —N(R′)SO₂R′, wherein R′ is as described herein. In some embodiments, -X—R^{L} is —NHSO₂R′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in -SO₂R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, —X—R^{L} is —NHSO₂CH₃. In some embodiments, R is —CF₃. In some embodiments, R is methyl. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted propyl. In some embodiments, R is optionally substituted butyl. In some embodiments, R is n-butyl. In some embodiments, R is -(CH₂)6NH₂. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments. R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

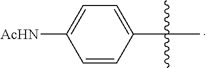 [SMILES: *c1ccc(NC(C)=O)cc1]

In some embodiments, R is

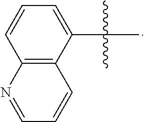 [SMILES: *c1cccc2ncccc12]

In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R is isopropyl. In some embodiments, R″ is —N(R′)₂. In some embodiments, R″ is —N(CH₃)₂. In some embodiments, R″, e.g., in —SO₂R″, is —OR′, wherein R′ is as described herein. In some embodiments, R′ is R as described herein. In some embodiments, R″ is —OCH₃. In some embodiments, a linkage is —OP(═O)(-NHSO₂R)O—, wherein R is as described herein. In some embodiments, R is optionally substituted linear alkyl as described herein. In some embodiments, R is linear alkyl as described herein. In some embodiments, a linkage is —OP(═O)(-NHSO₂CH₃)O—. In some embodiments, a linkage is —OP(═O)(-NHSO₂CH₂CH₃)O—. In some embodiments, a linkage is —OP(═O)(-NHSO₂CH₂CH₂OCH₃)O—. In some embodiments, a linkage is —OP(═O)(-NHSO₂CH₂Ph)O—. In some embodiments, a linkage is —OP(═O)(-NHSO₂CH₂CHF₂)O—. In some embodiments, a linkage is —OP(═O)(-NHSO₂(4-methylphenyl))O—. In some embodiments, —X—R^{L} is

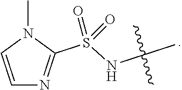 [SMILES: *NS(=O)(=O)c1nccn1C]

In some embodiments, a linkage is —OP(═O)(—X—R^{L})O—, wherein —X—R^{L} is

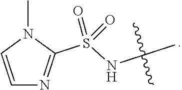 [SMILES: *NS(=O)(=O)c1nccn1C]

In some embodiments, a linkage is —OP(═O)(-NHSO₂CH(CH₃)₂)O—. In some embodiments, a linkage is -OP(═O)(-NHSO₂N(CH₃)₂)O—. In some embodiments, a linkage is n002. In some embodiments, a linkage is n006. In some embodiments, a linkage is n020. In some embodiments, such internucleotidic linkages may be utilized in place of linkages like n001.

In some embodiments, an internucleotidic linkage has the structure of -P(═O)(-N(R′)C(O)R″)-, wherein R″ is as described herein. In some embodiments, an internucleotidic linkage has the structure of -P(═S)(-N(R′)C(O)R″)-, wherein R″ is as described herein. In some embodiments, an internucleotidic linkage has the structure of -P(═O)(-N(R′)C(O)R″)O—, wherein R″ is as described herein. In some embodiments, an internucleotidic linkage has the structure of -P(═S)(-N(R′)C(O)R″)O—, wherein R″ is as described herein. In some embodiments, an internucleotidic linkage has the structure of -OP(═O)(-N(R′)C(O)R″)O—, wherein R″ is as described herein. In some embodiments, an internucleotidic linkage has the structure of —OP(═S)(-N(R′)C(O)R″)O—, wherein R″ is as described herein. In some embodiments, R′, e.g.. of N(R′), is hydrogen or optionally substituted C₁₋₆ aliphatic. In some embodiments, R′ is C₁₋₆ alkyl. In some embodiments, R′ is hydrogen. In some embodiments, R″, e.g., in —C(O)R″, is R′ as described herein. In some embodiments, an internucleotidic linkage has the structure of -P(═O)(-NHC(O)R″)-, wherein R″ is as described herein. In some embodiments, an internucleotidic linkage has the structure of -P(═S)(-NHC(O)R″)-, wherein R″ is as described herein. In some embodiments, an internucleotidic linkage has the structure of -P(═O)(-NHC(O)R″)O—, wherein R″ is as described herein. In some embodiments, an internucleotidic linkage has the structure of P(═S)(-NHC(O)R″)O—, wherein R″ is as described herein. In some embodiments, an internucleotidic linkage has the structure of -OP(═O)(-NHC(O)R″)O—, wherein R″ is as described herein. In some embodiments, an internucleotidic linkage has the structure of —OP(═S)(-NHC(O)R″)O—, wherein R″ is as described herein. In some embodiments, —X—R^{L} is —N(R′)COR^{L}, wherein R^{L} is as described herein. In some embodiments, —X—R^{L} is —N(R′)COR″, wherein R″ is as described herein. In some embodiments, —X—R^{L} is —N(R′)COR′, wherein R′ is as described herein. In some embodiments, —X—R^{L} is NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —C(O)R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, —X—R^{L} is —NHC(O)CH₃. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C_{2_6}, C₃₋₆, C_{1_10}, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R^{L} is -(CH₂)₅NH₂. In some embodiments, R^{L} is

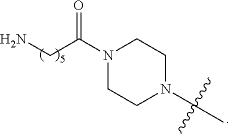

In some embodiments, R^{L} is

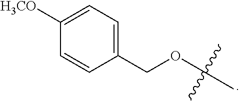 [SMILES: *OCc1ccc(OC)cc1]

In some embodiments, R″ is —N(R′)₂. In some embodiments, R″ is —N(CH₃)₂. In some embodiments, —X—R^{L} is —N(R′)CON(R^{L})₂, wherein each of R′ and R^{L} is independently as described herein. In some embodiments, —X—R^{L} is -NHCON(R^{L})₂, wherein R^{L} is as described herein. In some embodiments, two R′ or two R^{L} are taken together with the nitrogen atom to which they are attached to form a ring as described herein, e.g., optionally substituted

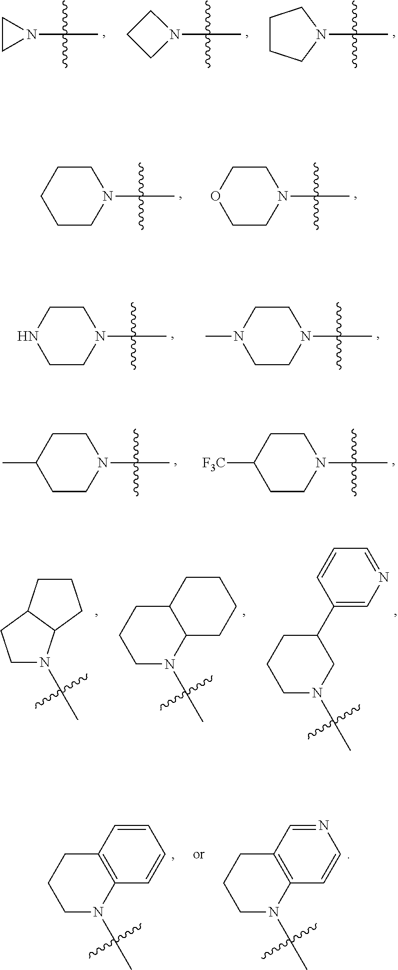

In some embodiments, R″, e.g., in —C(O)R″, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, -X—R^{L} is —N(R′)C(O)OR^{L}, wherein each of R′ and R^{L} is independently as described herein. In some embodiments, R is

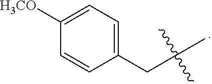 [SMILES: *Cc1ccc(OC)cc1]

In some embodiments, —X—R^{L} is —NHC(O)OCH₃. In some embodiments, —X—R^{L} is —NHC(O)N(CH₃)₂. In some embodiments, a linkage is —OP(O)(NHC(O)CH₃)O—. In some embodiments, a linkage is —OP(O)(NHC(O)OCH₃)O—. In some embodiments, a linkage is —OP(O)(NHC(O)(p-methylphenyl))O—. In some embodiments, a linkage is —OP(O)(NHC(O)N(CH₃)₂)O—. In some embodiments, —X—R^{L} is -N(R′)R^{L}, wherein each of R′ and R^{L} is independently as described herein. In some embodiments, —X—R^{L} is -N(R′)R^{L}, wherein each of R′ and R^{L} is independently not hydrogen. In some embodiments, —X—R^{L} is -NHRL, wherein R^{L} is as described herein. In some embodiments, R^{L} is not hydrogen. In some embodiments, R^{L} is optionally substituted aryl or heteroaryl. In some embodiments, R^{L} is optionally substituted aryl. In some embodiments, R^{L} is optionally substituted phenyl. In some embodiments, —X—R^{L} is —N(R′)₂, wherein each R′ is independently as described herein. In some embodiments, —X—R^{L} is —NHR′, wherein R′ is as described herein. In some embodiments, —X—R^{L} is —NHR, wherein R is as described herein. In some embodiments, -X—R^{L} is R^{L}, wherein R^{L} is as described herein. In some embodiments, R^{L} is —N(R′)₂, wherein each R′ is independently as described herein. In some embodiments. R^{L} is —NHR′, wherein R′ is as described herein. In some embodiments, R^{L} is —NHR, wherein R is as described herein. In some embodiments, R^{L} is —N(R′)₂, wherein each R′ is independently as described herein. In some embodiments, none of R′ in -N(R′)₂ is hydrogen. In some embodiments, R^{L} is —N(R′)₂, wherein each R′ is independently C₁₋₆ aliphatic. In some embodiments, R^{L} is -L-R′, wherein each of L and R′ is independently as described herein. In some embodiments, R^{L} is -L-R, wherein each of L and R is independently as described herein. In some embodiments, R^{L} is —N(R′)—Cy-N(R′)-R′. In some embodiments, R^{L} is —N(R′)—Cy-C(O)-R′. In some embodiments, R^{L} is —N(R′)—Cy-O-R′. In some embodiments, R^{L} is —N(R′)—Cy-SO₂—R′. In some embodiments, R^{L} is —N(R′)—Cy-SO₂-N(R′)₂. In some embodiments, R^{L} is —N(R′)—Cy-C(O)-N(R′)₂. In some embodiments, R^{L} is —N(R′)—Cy-OP(O)(R″)₂. In some embodiments, —Cy— is an optionally substituted bivalent aryl group. In some embodiments, —Cy— is optionally substituted phenylene. In some embodiments, -Cy— is optionally substituted 1,4-phenylene. In some embodiments, —Cy— is 1,4-phenylene. In some embodiments, R^{L} is —N(CH₃)₂. In some embodiments, R^{L} is —N(i-Pr)₂. In some embodiments, R^{L} is

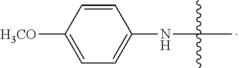 [SMILES: *Nc1ccc(OC)cc1]

In some embodiments, R^{L} is

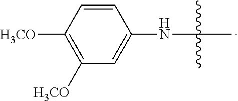 [SMILES: *Nc1ccc(OC)c(OC)c1]

In some embodiments, R^{L} is

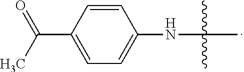 [SMILES: *Nc1ccc(C(C)=O)cc1]

In some embodiments, R^{L} is

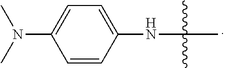 [SMILES: *Nc1ccc(N(C)C)cc1]

In some embodiments, R^{L} is

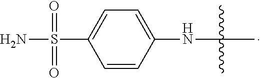 [SMILES: *Nc1ccc(S(N)(=O)=O)cc1]

In some embodiments, R^{L} is

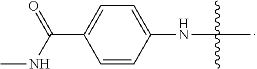 [SMILES: *Nc1ccc(C(=O)NC)cc1]

In some embodiments, R^{L} is

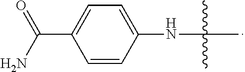 [SMILES: *Nc1ccc(C(N)=O)cc1]

In some embodiments, R^{L} is

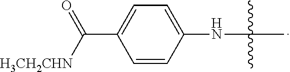

In some embodiments, R^{L}is

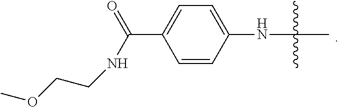 [SMILES: *Nc1ccc(C(=O)NCCOC)cc1]

In some embodiments, R^{L} is

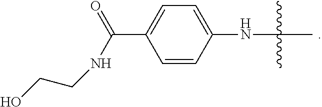 [SMILES: *Nc1ccc(C(=O)NCCO)cc1]

In some embodiments, R^{L} is

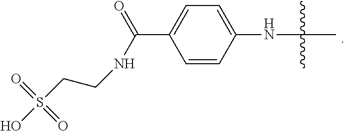 [SMILES: *Nc1ccc(C(=O)NCCS(=O)(=O)O)cc1]

In some embodiments, R^{L} is

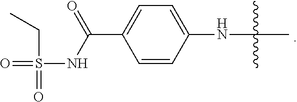 [SMILES: *Nc1ccc(C(=O)NS(=O)(=O)CC)cc1]

In some embodiments, R^{L} is

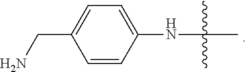 [SMILES: *Nc1ccc(CN)cc1]

In some embodiments, R^{L} is

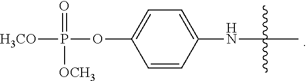 [SMILES: *Nc1ccc(OP(=O)(OC)OC)cc1]

In some embodiments, R^{L} is

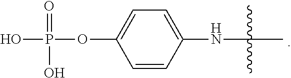 [SMILES: *Nc1ccc(OP(=O)(O)O)cc1]

In some embodiments, R^{L} is

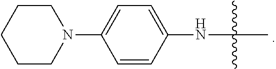 [SMILES: *Nc1ccc(N2CCCCC2)cc1]

In some embodiments, R^{L} is

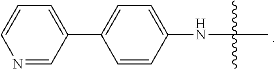 [SMILES: *Nc1ccc(-c2cccnc2)cc1]

In some embodiments, R^{L} is

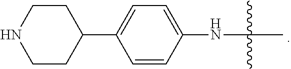 [SMILES: *Nc1ccc(C2CCNCC2)cc1]

In some embodiments, R^{L}is

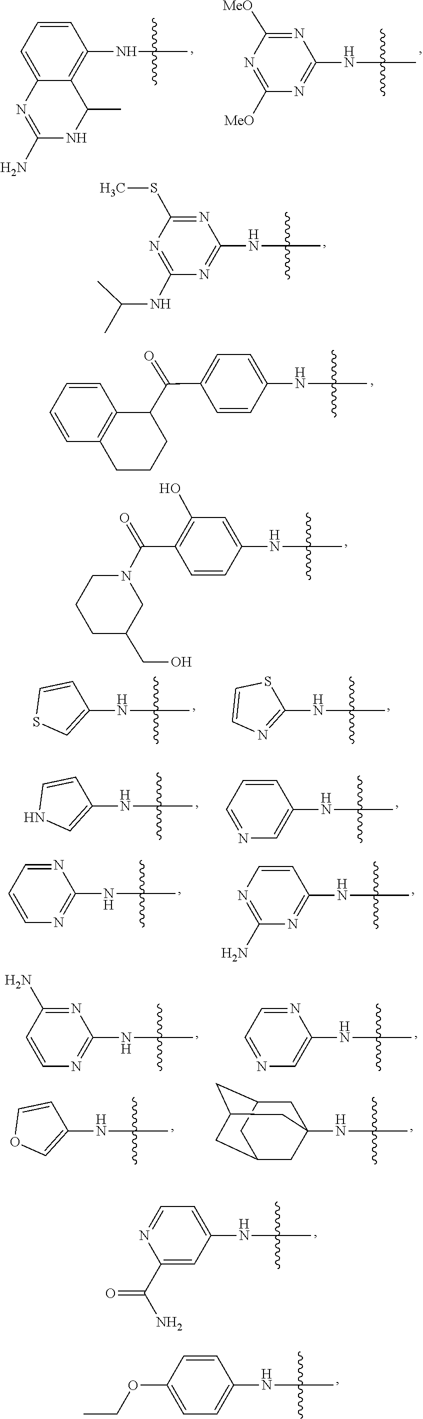

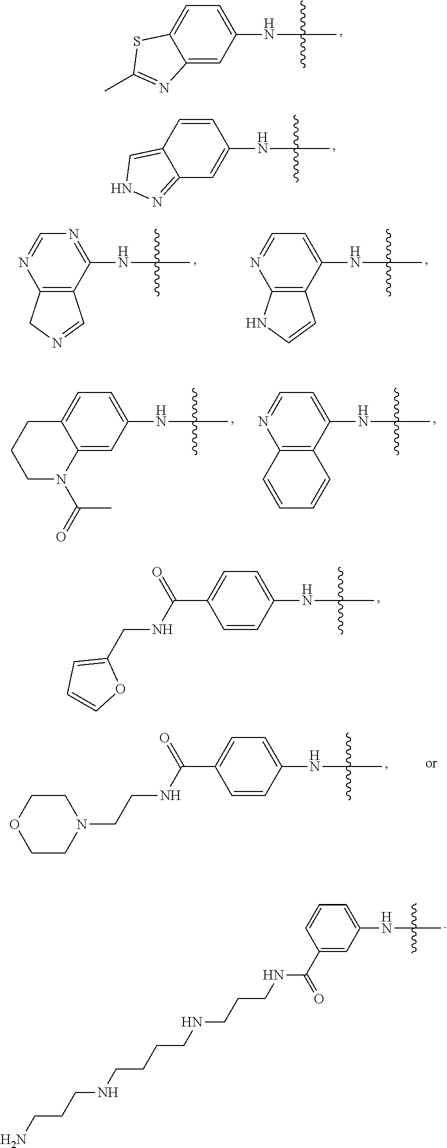

In some embodiments, -X—R^{L} is N(R′)-C(O)-Cy-R^{L}. In some embodiments, —X—R^{L} is R^{L}. In some embodiments, R^{L} is —N(R′)—C(O)-Cy-O-R′. In some embodiments, R^{L} is —N(R′)—C(O)-Cy-R′. In some embodiments, R^{L} is —N(R′)—C(O)-Cy-C(O)-R′. In some embodiments, R^{L} is —N(R′)—C(O)-Cy-N(R′)₂. In some embodiments, R^{L} is —N(R′)—C(O)-Cy-SO₂-N(R′)₂. In some embodiments, R^{L} is —N(R′)—C(O)-Cy-C(O)-N(R′)₂. In some embodiments, R^{L} is —N(R′)—C(O)-Cy-C(O)-N(R′)—SO₂—R′. In some embodiments, R′ is R as described herein. In some embodiments, R^{L} is

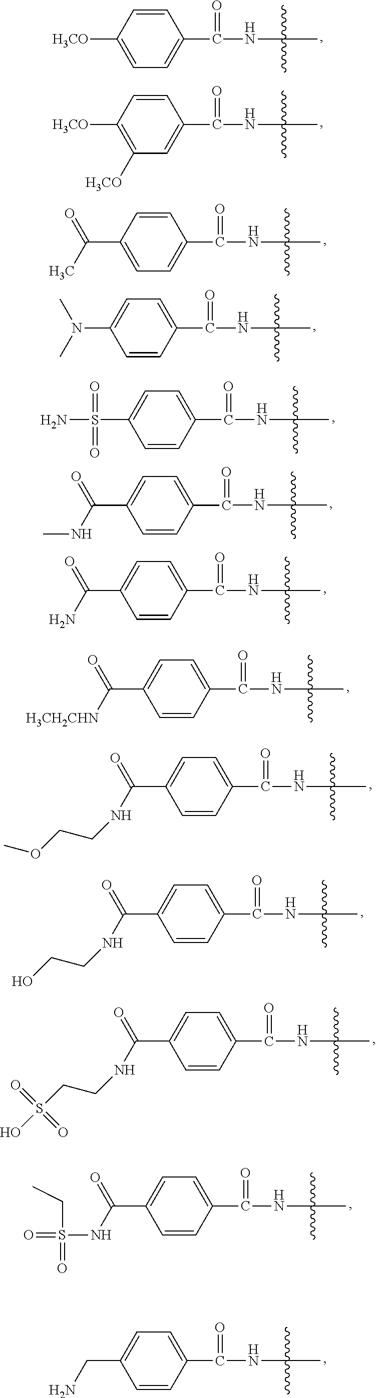

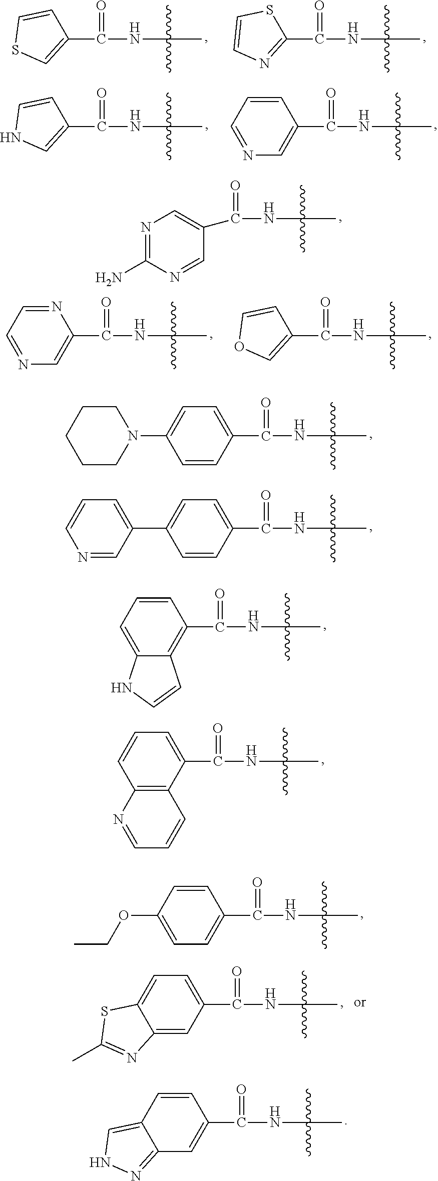

As described herein, in some embodiments, one or more methylene units of L, or a variable which comprises or is L. are independently replaced with —O—, —N(R′)—, —C(O)—, —C(O)N(R′)—, —SO₂—, SO₂N(R′)—, or —Cy—. In some embodiments, amethylene unit is replaced with —Cy-. In some embodiments, —Cy— is an optionally substituted bivalent aryl group. In some embodiments, —Cy— is optionally substituted phenylene. In some embodiments, —Cy— is optionally substituted 1,4-phenylene. In some embodiments, —Cy-is an optionally substituted bivalent 5-20 (e.g. 5, 6, 7, 8, 9, 10, 11, 12, 13, 14, 15, 16, 17, 18, 19, or 20) membered heteroaryl group having 1-10 (e.g., 1, 2, 3, 4, 5, 6, 7, 8, 9, or 10) heteroatoms. In some embodiments, -Cy— is monocyclic. In some embodiments, —Cy— is bicyclic. In some embodiments. —Cy— is polycyclic. In some embodiments, each monocyclic unit in —Cy— is independently 3-10 (e.g., 3, 4, 5, 6, 7, 8, 9, or 10) membered, and is independently saturated, partially saturated, or aromatic. In some embodiments, —Cy— is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic aliphatic group. In some embodiments, —Cy— is an optionally substituted 3-20 (e.g., 3, 4, 5, 6,7, 8, 9, 10, 11, 12, 13, 14, 15, 16, 17, 18, 19, or 20) membered monocyclic, bicyclic or polycyclic heteroaliphatic group having 1-10 (e.g., 1, 2, 3, 4, 5, 6, 7, 8, 9, or 10) heteroatoms.

In some embodiments, an internucleotidic linkage has the structure of -P(═O)(-N(R′)P(O)(R″)₂)-, wherein each R″ is independently as described herein. In some embodiments, an internucleotidic linkage has the structure of -P(═S)(-N(R′)P(O)(R″)₂)-, wherein each R″ is independently as described herein. In some embodiments, an internucleotidic linkage has the structure of -P(═O)(-N(R′)P(O)(R″)₂)O—, wherein each R″ is independently as described herein. In some embodiments, an internucleotidic linkage has the structure of -P(═S)(-N(R′)P(O)(R″)₂)O—, wherein each R″ is independently as described herein. In some embodiments, an internucleotidic linkage has the structure of -OP(═O)(-N(R′)P(O)(R″)₂)O—, wherein each R″ is independently as described herein. In some embodiments, an internucleotidic linkage has the structure of —OP(═S)(-N(R′)P(O)(R″)₂)O—, wherein each R″ is independently as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in -P(O)(R″)₂, is R′ as described herein. In some embodiments, an internucleotidic linkage has the structure of -P(═O)(-NHP(O)(R″)₂)-, wherein each R″ is independently as described herein. In some embodiments, an internucleotidic linkage has the structure of -P(═S)(-NHP(O)(R″)₂)-, wherein each R″ is independently as described herein. In some embodiments, an internucleotidic linkage has the structure of -P(═O)(-NHP(O)(R″)₂)O—, wherein each R″ is independently as described herein. In some embodiments, an internucleotidic linkage has the structure of -P(═S)(-NHP(O)(R″)₂)O—, wherein each R″ is independently as described herein. In some embodiments, an internucleotidic linkage has the structure of —OP(═O)(-NHP(O)(R″)₂)O—, wherein each R″ is independently as described herein. In some embodiments, an internucleotidic linkage has the structure of -OP(═S)(-NHP(O)(R″)₂)O—, wherein each R″ is independently as described herein. In some embodiments, an occurrence of R″, e.g., in -P(O)(R″)₂, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is isopropyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, Cs, C₉, C₁₀, C₁₁, C₁₂, C₁₃, C₁₄, C₁₅, C₁₆, C₁₇, C₁s, C₁₉, or C₂₀ alkyl. In some embodiments, R is optionally substituted linear C₁, C₂, C₃, C₄, C₅, C₆, C₇, Cs, C₉, C₁₀, C₁₁, C₁₂, C₁₃, C₁₄, C₁₅, C₁₆, C₁₇, Cis, C₁₉, or C₂₀ alkyl. In some embodiments, R is linear C₁, C₂, C₃, C₄, C₅, C₆, C₇, Cs, C₉, C₁₀, C₁₁, C₁₂, C₁₃, C₁₄, C₁₅, C₁₆, C₁₇, C₁s, C₁₉, or C₂₀ alkyl. In some embodiments, each R″ is independently R as described herein, for example, in some embodiments, each R″ is methyl. In some embodiments, R″ is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, an occurrence of R″ is —N(R′)₂. In some embodiments, R″ is —N(CH₃)₂. In some embodiments, an occurrence of R″, e.g., in -P(O)(R″)₂, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, each R″ is —OR′ as described herein. In some embodiments, each R″ is —OCH₃. In some embodiments, each R′ is —OH. In some embodiments, a linkage is —OP(O)(NHP(O)(OH)₂)O—. In some embodiments, a linkage is —OP(O)(NHP(O)(OCH₃)₂)O—. In some embodiments, a linkage is —OP(O)(NHP(O)(CH₃)₂)O—.

In some embodiments, —N(R″)₂ is —N(R′)₂. In some embodiments, —N(R″)₂ is —NHR. In some embodiments, —N(R″)₂ is —NHC(O)R. In some embodiments, —N(R″)₂ is —NHC(O)OR. In some embodiments, —N(R″)₂ is —NHS(O)₂R.

In some embodiments, an internucleotidic linkage is a phosphoryl guanidine internucleotidic linkage. In some embodiments, an internucleotidic linkage comprises —X—R^{L} as described herein. In some embodiments, —X—R^{L} is —N═C(-L^{L}-R^{L})₂. In some embodiments, —X—R^{L} is —N═C[N(R^{L})₂]₂ In some embodiments, —X—R^{L} is —N═C[NR′R^{L}₂. In some embodiments, —X—R^{L} is —N═C[N(R′)₂]2. In some embodiments, —X—R^{L} is —N═C[N(R^{L})₂](CHR′R^{L2}), wherein each of R^{L} and R^{L} is independently as described herein. In some embodiments, —X—R^{L} is —N═C(NR′R^{L})(CHR′R^{L2}), wherein each of R^{L}L and R^{L2} is independently as described herein. In some embodiments, —X—R^{L} is —N=C(NR′R^{L})(CR′R^{L}′R^{L}), wherein each of R^{L1} and R^{L2} is independently as described herein. In some embodiments, —X—R^{L} is -N═C[N(R′)₂](CHR′R^{L}). In some embodiments, —X—R^{L} is —N═C[N(R^{L})₂](R^{L}). In some embodiments, —X—R^{L} is —N═C(NR′R^{L})(R^{L}). In some embodiments, —X—R^{L} is —N═C(NR′R^{L})(R,). In some embodiments, —X—R^{L} is —N═C[N(R′)₂](R′). In some embodiments, —X—R^{L} is —N═C(NR′R^{L1})(NR′R^{L2}), wherein each R^{L1} and R^{L} is independently R^{L}, and each R′ and R^{L} is independently as described herein. In some embodiments, —X—R^{L} is —N=C(NR′R^{L1})(NR′R^{L}), wherein variable is independently as described herein. In some embodiments, —X—R^{L} is —N=C(NR′R^{L})(CHR′R^{L}), wherein variable is independently as described herein. In some embodiments, —X—R^{L} is —N═C(NR′R^{L1})(R′), wherein variable is independently as described herein. In some embodiments, each R′ is independently R. In some embodiments, R is optionally substituted C₁₋₆ aliphatic. In some embodiments, R is methyl. In some embodiments, —X—R^{L} is

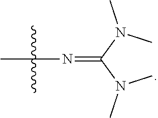 [SMILES: *N=C(N(C)C)N(C)C]

In some embodiments, two groups selected from R′, R^{L}, R^{L1}, R^{L2}, etc. (in some embodiments, on the same atom (e.g., —N(R′)₂, or —NR′R^{L}, or —N(R^{L})₂, wherein R′ and R^{L} can independently be R as described herein), etc.), or on different atoms (e.g., the two R′ in —N═C(NR′R^{L})(CR′R^{L1}R^{L2}) or —N═C(NR′R^{L1})(NR′R^{L2}); can also be two other variables that can be R, e.g., R^{L}, R^{L1}, R^{L2}, etc.)) are independently R and are taken together with their intervening atoms to form a ring as described herein. In some embodiments, two of R, R′, R^{L}, R^{L1}, or R^{L2} on the same atom, e.g., of —N(R′)₂, —N(R^{L})₂, —NR′R^{L}, —NR′R^{L1}, —NRR^{L2}, —CR′R^{L1}R^{L2}, etc., are taken together to form a ring as described herein. In some embodiments, two R′, R^{L}, R^{L1}, or R^{L2} on two different atoms, e.g., the two R′ in —N═C(NR′R^{L1})(CR′R^{L2}), —N═C(NR′R^{L1})(NR′R^{L2}), etc. are taken together to form a ring as described herein. In some embodiments, a formed ring is an optionally substituted 3-20 (e.g., 3-15, 3-12, 3-10, 3-9, 3-8, 3-7, 3-6, 4-15, 4-12, 4-10, 4-9, 4-8, 4-7, 4-6, 5-15, 5-12, 5-10, 5-9, 5-8, 5-7, 5-6, 1, 2, 3, 4, 5, 6, 7, 8, 9, 10, 11, 12, 13, 14, 15, 16, 17, 18, 19, 20, etc.) monocyclic, bicyclic or tricyclic ring having 0-5 additional heteroatoms. In some embodiments, a formed ring is monocyclic as described herein. In some embodiments, a formed ring is an optionally substituted 5-10 membered monocyclic ring. In some embodiments, a formed ring is bicyclic. In some embodiments, a formed ring is polycyclic. In some embodiments, two groups that are or can be R (e.g., the two R′ in —N═C(NR′R^{L1})(CR′R^{L2}) or —N═C(NR′R^{L1})(NR′R^{L2}), the two R′ in -N═C(NR′R^{L1})(CR′R^{L2}), —N═C(NR′R^{L1})(NR′R^{L2}), etc.) are taken together to form an optionally substituted bivalent hydrocarbon chain, e.g., an optionally substituted C-₂₀ aliphatic chain, optionally substituted —(CH₂)n- wherein n is 1-20 (e.g., 1, 2, 3, 4, 5, 6, 7, 8, 9, 10, 11, 12, 13, 14, 15, 16, 17, 18, 19, or 20). In some embodiments, a hydrocarbon chain is saturated. In some embodiments, a hydrocarbon chain is partially unsaturated. In some embodiments, a hydrocarbon chain is unsaturated. In some embodiments, two groups that are or can be R (e.g., the two R′ in —N═C(NR′R^{L1})(CR′R^{L1}R^{L2}) or —N═C(NR′R^{L1})(NR′R^{L2}), the two R′ in —N═C(NR′R^{L1})(CR′R^{L1}R^{L2}), —N═C(NR′R^{L1})(NR′R^{L2}), etc.) are taken together to form an optionally substituted bivalent heteroaliphatic chain, e.g., an optionally substituted C₁₋₂₀ heteroaliphatic chain having 1-10 heteroatoms. In some embodiments, a heteroaliphatic chain is saturated. In some embodiments, a heteroaliphatic chain is partially unsaturated. In some embodiments, a heteroaliphatic chain is unsaturated. In some embodiments, a chain is optionally substituted —(CH₂)—. In some embodiments, a chain is optionally substituted —(CH₂)₂—. In some embodiments, a chain is optionally substituted —(CH₂)—. In some embodiments, a chain is optionally substituted —(CH₂)₂—. In some embodiments, a chain is optionally substituted —(CH₂)₃—. In some embodiments, a chain is optionally substituted —(CH₂)₄—. In some embodiments, a chain is optionally substituted —(CH₂)₅—. In some embodiments, a chain is optionally substituted —(CH₂)₆—. In some embodiments, a chain is optionally substituted —CH═CH—. In some embodiments, a chain is optionally substituted

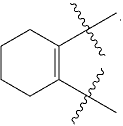 [SMILES: *C1=C(*)CCCC1]

In some embodiments, a chain is optionally substituted

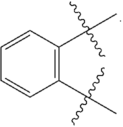 [SMILES: *c1ccccc1*]

In some embodiments, a chain is optionally substituted

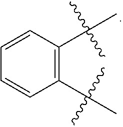 [SMILES: *c1ccccc1*]

In some embodiments, a chain is optionally substituted

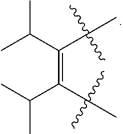 [SMILES: */C(=C(\*)C(C)C)C(C)C]

In some embodiments, a chain is optionally substituted

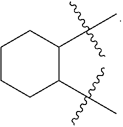 [SMILES: *C1CCCCC1*]

In some embodiments, a chain is optionally substituted

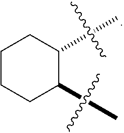

In some embodiments, a chain is optionally substituted

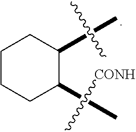

In some embodiments, a chain is optionally substituted

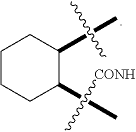

In some embodiments, a chain is optionally substituted

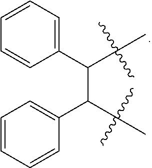 [SMILES: *C(c1ccccc1)C(*)c1ccccc1]

In some embodiments, two of R, R′, R^{L}, R^{L1}, R^{L2}, etc. on different atoms are taken together to form a ring as described herein. For examples, in some embodiments, —X—R^{L} is

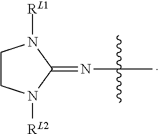

In some embodiments, —X—R^{L} is

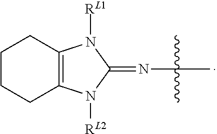

In some embodiments, —X—R^{L} is

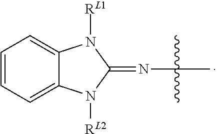

In some embodiments, —X—R^{L} is

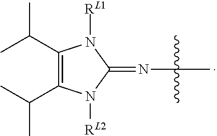

In some embodiments, —X—R^{L} is

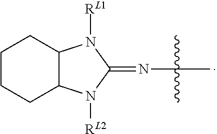

In some embodiments, —X—R^{L} is

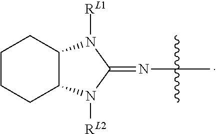

In some embodiments, —X—R^{L} is

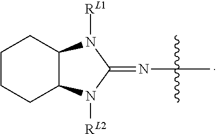

In some embodiments, —X—R^{L} is

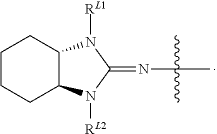

In some embodiments, —X—R^{L} is

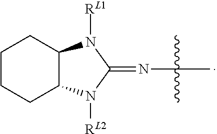

In some embodiments, —X—R^{L} is

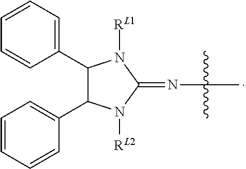

In some embodiments, —N(R′)₂, —N(R)₂, —N(R^{L})₂, —NR′R^{L}, —NR′R^{L1}, —NR′R^{L2}, —NR^{L1}R^{L2}, etc. is a formed ring. In some embodiments, a ring is optionally substituted

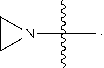 [SMILES: *N1CC1]

In some embodiments, a ring is optionally substituted

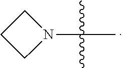 [SMILES: *N1CCC1]

In some embodiments, a ring is optionally substituted

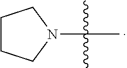 [SMILES: *N1CCCC1]

In some embodiments, a ring is optionally substituted

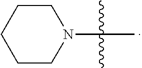 [SMILES: *N1CCCCC1]

In some embodiments, a ring is optionally substituted

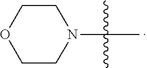 [SMILES: *N1CCOCC1]

In some embodiments, a ring is optionally substituted

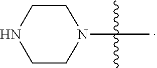 [SMILES: *N1CCNCC1]

In some embodiments, a ring is optionally substituted

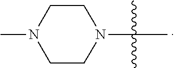 [SMILES: *N1CCN(C)CC1]

In some embodiments, a ring is optionally substituted

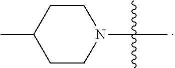 [SMILES: *N1CCC(C)CC1]

In some embodiments, a ring is optionally substituted

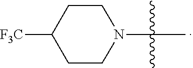 [SMILES: *N1CCC(C(F)(F)F)CC1]

In some embodiments, a ring is optionally substituted

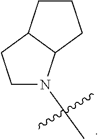 [SMILES: *N1CCC2CCCC21]

In some embodiments, a ring is optionally substituted

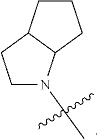 [SMILES: *N1CCC2CCCC21]

In some embodiments, a ring is optionally substituted

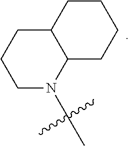 [SMILES: *N1CCCC2CCCCC21]

In some embodiments, a ring is optionally substituted

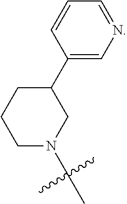 [SMILES: *N1CCCC(c2cccnc2)C1]

In some embodiments, a ring is optionally substituted

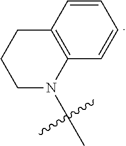 [SMILES: *N1CCCc2ccccc21]

In some embodiments, a ring is optionally substituted

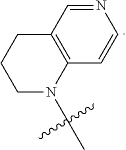 [SMILES: *N1CCCc2cnccc21]

In some embodiments, R^{L1} and R^{L2} are the same. In some embodiments, R^{L1} and R^{L2} are different. In some embodiments, each of R^{L1} and R^{L2} is independently R^{L} as described herein, e.g., below.

In some embodiments, R^{L} is optionally substituted C₁₃₀ aliphatic. In some embodiments, R^{L} is optionally substituted C₁₋₃₀ alkyl. In some embodiments, R^{L} is linear. In some embodiments, R^{L} is optionally substituted linear C₁₋₃₀ alkyl. In some embodiments, R^{L} is optionally substituted C₁₋₆ alkyl. In some embodiments, R^{L} is methyl. In some embodiments, R^{L} is ethyl. In some embodiments, R^{L} is n-propyl. In some embodiments, R^{L} is isopropyl. In some embodiments, R^{L} is n-butyl. In some embodiments, R^{L} is tert-butyl. In some embodiments, R^{L} is (E)-CH₂—CH═CH—CH₂—CH₃. In some embodiments, R^{L} is (Z)-CH₂—CH═CH—CH₂—CH₃. In some embodiments, R^{L} is

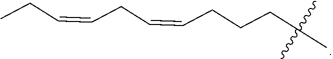 [SMILES: *CCC/C=C\C/C=C\CC]

In some embodiments, R^{L} is

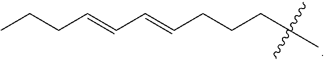 [SMILES: *CCC/C=C/C=C/CCC]

In some embodiments, R^{L} is CH₃(CH₂)₂C≡CC≡C(CH₂)₃-. In some embodiments, R^{L} is CH₃(CH₂)₅C≡C—. In some embodiments, R^{L} optionally substituted aryl. In some embodiments, R^{L} is optionally substituted phenyl. In some embodiments, R^{L} is phenyl substituted with one or more halogen. In some embodiments, R^{L} is phenyl optionally substituted with halogen, —N(R′), or —N(R′)C(O)R′. In some embodiments, R^{L} is phenyl optionally substituted with —Cl, —Br, —F, —N(Me)₂, or —NHCOCH₃. In some embodiments, R^{L} is -L^{L}-R′, wherein L^{L} is an optionally substituted C₁₋₂₀ saturated, partially unsaturated or unsaturated hydrocarbon chain. In some embodiments, such a hydrocarbon chain is linear. In some embodiments, such a hydrocarbon chain is unsubstituted. In some embodiments, L^{L}is (E)-CH₂—CH═CH—. In some embodiments, L^{L} is —CH₂—C═C—CH₂—. In some embodiments, L^{L} is -(CH₂)₃—. In some embodiments, L^{L} is -(CH₂)₄-. In some embodiments, L^{L} is -(CH₂)ₙ-, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R′ is optionally substituted aryl as described herein. In some embodiments, R′ is optionally substituted phenyl. In some embodiments, R′ is phenyl. In some embodiments, R′ is optionally substituted heteroaryl as described herein. In some embodiments, R′ is 2′-pyridinyl. In some embodiments, R′ is 3′-pyridinyl. In some embodiments, R^{L} is

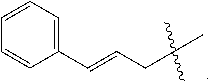 [SMILES: *C/C=C/c1ccccc1]

In some embodiments, R^{L} is

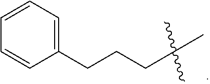 [SMILES: *CCCc1ccccc1]

In some embodiments, R^{L} is

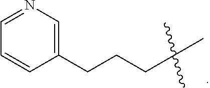 [SMILES: *CCCc1cccnc1]

In some embodiments, R^{L} is -L^{L}-N(R′)₂, wherein each variable is independently as described herein. In some embodiments, each R′ is independently C₁₋₆ aliphatic as described herein. In some embodiments, —N(R′)₂ is —N(CH₃)₂. In some embodiments, —N(R′)₂ is —NH₂. In some embodiments, R^{L} is -(CH₂)ₙ-N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is -(CH₂CH₂O)ₙ-CH₂CH₂—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is

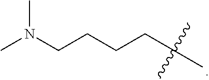 [SMILES: *CCCCN(C)C]

In some embodiments, R^{L} is

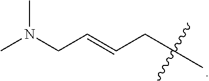 [SMILES: *C/C=C/CN(C)C]

In some embodiments, R^{L} is

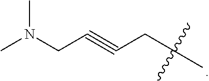 [SMILES: *CC#CCN(C)C]

In some embodiments, R^{L} is —(CH₂)—NH₂. In some embodiments, R^{L} is -(CH₂CH₂O)ₙ-CH₂CH₂—NH₂. In some embodiments, R^{L} is -(CH₂CH₂O)ₙ-CH₂CH₂—R′, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is -(CH₂CH₂O)—CH₂CH₂CH₃, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is -(CH₂CH₂O)ₙ—CH₂CH₂OH, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is or comprises a carbohydrate moiety, e.g., GalNAc. In some embodiments, R^{L} is -L^{L}-GalNAc. In some embodiments, R^{L} is

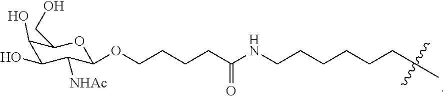 [SMILES: *CCCCCCNC(=O)CCCCOC1OC(CO)C(O)C(O)C1NC(C)=O]

In some embodiments, one or more methylene units of L^{L} are independently replaced with —Cy— (e.g., optionally substituted 1,4-phenylene, a 3-30 membered bivalent optionally substituted monocyclic, bicyclic, or polycyclic cycloaliphatic ring, etc.), —O—, —N(R′)- (e.g., —NH), —C(O)—, —C(O)N(R′)- (e.g., —C(O)NH—), —C(NR′)-(e.g., —C(NH)—), —N(R′)C(O)(N(R′)- (e.g., —NHC(O)NH—), —N(R′)C(NR′)(N(R′)- (e.g., —NHC(NH)NH—), -(CH₂CH₂O)ₙ-, etc. For example, in some embodiments, R^{L} is

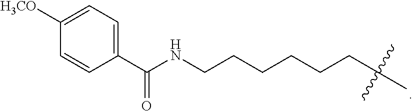 [SMILES: *CCCCCCNC(=O)c1ccc(OC)cc1]

In some embodiments. R^{L} is

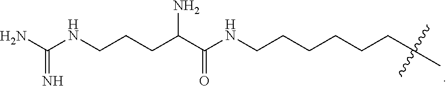 [SMILES: *CCCCCCNC(=O)C(N)CCCNC(=N)N]

In some embodiments, R^{L} is

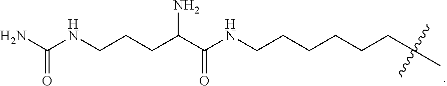 [SMILES: *CCCCCCNC(=O)C(N)CCCNC(N)=O]

In some embodiments, R^{L} is

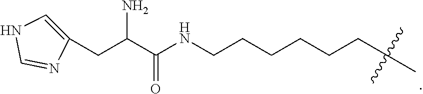 [SMILES: *CCCCCCNC(=O)C(N)Cc1c[nH]cn1]

In some embodiments, R^{L} is

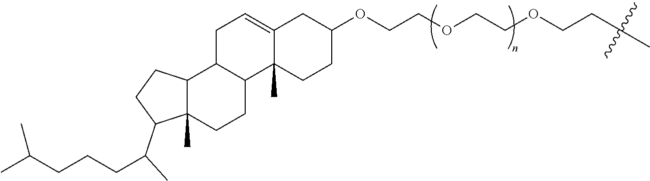

wherein n is 0-20. In some embodiments, R^{L} is or comprises one or more additional chemical moieties (e.g., carbohydrate moieties, GalNAc moieties, etc.) optionally substituted connected through a linker (which can be bivalent or polyvalent). For example, in some embodiments, R^{L} is

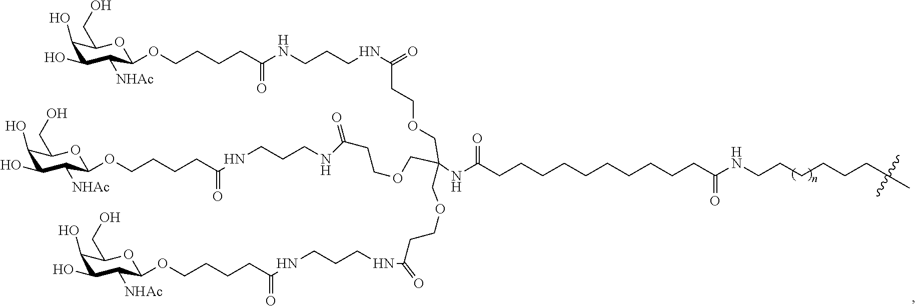

wherein n is 0-20. In some embodiments, R^{L} is

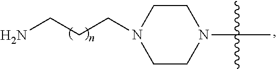

wherein n is 0-20. In some embodiments, R^{L} is R′ as described herein. As described herein, many variable can independently be R′. In some embodiments, R′ is R as described herein. As described herein, various variables can independently be R. In some embodiments, R is optionally substituted C₁₋₆ aliphatic. In some embodiments, R is optionally substituted C₁₋₆ alkyl. In some embodiments, R is methyl. In some embodiments, R is optionally substituted cycloaliphatic. In some embodiments, R is optionally substituted cycloalkyl. In some embodiments, R is optionally substituted aryl. In some embodiments, R is optionally substituted phenyl. In some embodiments, R is optionally substituted heteroaryl. In some embodiments, R is optionally substituted heterocyclyl. In some embodiments, R is optionally substituted C₁₋₂₀ heterocyclyl having 1-5 heteroatoms, e.g., one of which is nitrogen. In some embodiments, R is optionally substituted

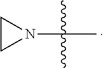 [SMILES: *N1CC1]

In some embodiments, R is optionally substituted

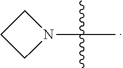 [SMILES: *N1CCC1]

In some embodiments, R is optionally substituted

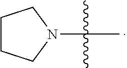 [SMILES: *N1CCCC1]

In some embodiments, R is optionally substituted

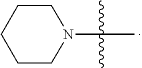 [SMILES: *N1CCCCC1]

In some embodiments, R is optionally substituted

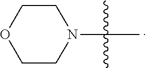 [SMILES: *N1CCOCC1]

In some embodiments, R is optionally substituted

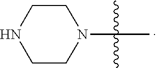 [SMILES: *N1CCNCC1]

In some embodiments, R is optionally substituted

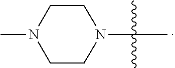 [SMILES: *N1CCN(C)CC1]

In some embodiments, R is optionally substituted

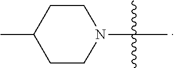 [SMILES: *N1CCC(C)CC1]

In some embodiments, R is optionally substituted

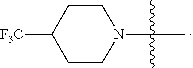 [SMILES: *N1CCC(C(F)(F)F)CC1]

In some embodiments, R is optionally substituted

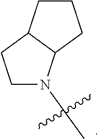 [SMILES: *N1CCC2CCCC21]

In some embodiments, R is optionally substituted

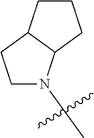 [SMILES: *N1CCC2CCCC21]

In some embodiments, R is optionally substituted

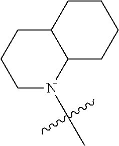 [SMILES: *N1CCCC2CCCCC21]

In some embodiments, R is optionally substituted

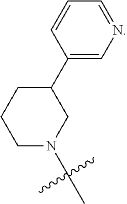 [SMILES: *N1CCCC(c2cccnc2)C1]

In some embodiments, R is optionally substituted

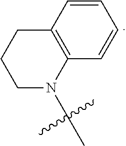 [SMILES: *N1CCCc2ccccc21]

In some embodiments, R is optionally substituted

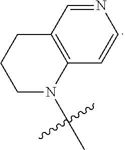 [SMILES: *N1CCCc2cnccc21]

In some embodiments, —X—R^{L} is

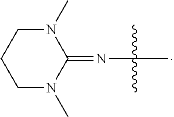 [SMILES: *N=C1N(C)CCCN1C]

In some embodiments, —X—R^{L} is

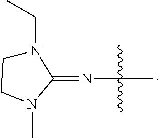 [SMILES: *N=C1N(C)CCN1CC]

In some embodiments, —X—R^{L} is

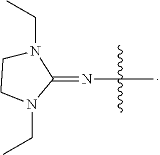 [SMILES: *N=C1N(CC)CCN1CC]

In some embodiments, —X—R^{L} is

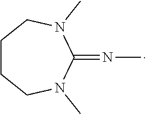

In some embodiments, -X—R^{L} is

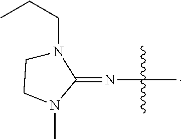 [SMILES: *N=C1N(C)CCN1CCC]

In some embodiments, -X—R^{L} is

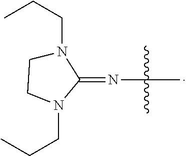 [SMILES: *N=C1N(CCC)CCN1CCC]

In some embodiments, -X—R^{L} is

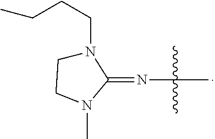 [SMILES: *N=C1N(C)CCN1CCCC]

In some embodiments, -X—R^{L} is

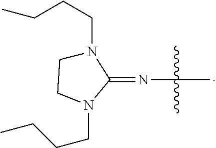 [SMILES: *N=C1N(CCCC)CCN1CCCC]

In some embodiments, —X—R^{L} is

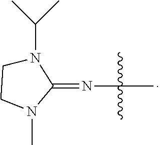 [SMILES: *N=C1N(C)CCN1C(C)C]

In some embodiments, —X—R^{L} is

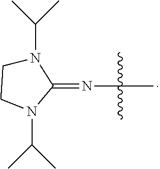 [SMILES: *N=C1N(C(C)C)CCN1C(C)C]

In some embodiments, -X—R^{L} is

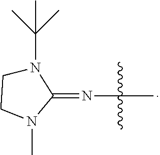 [SMILES: *N=C1N(C)CCN1C(C)(C)C]

In some embodiments, —X—R^{L} is

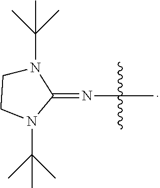 [SMILES: *N=C1N(C(C)(C)C)CCN1C(C)(C)C]

In some embodiments, -X—R^{L}

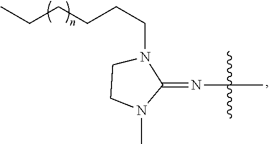

wherein n is 1-20. In some embodiments, -X—R^{L} is

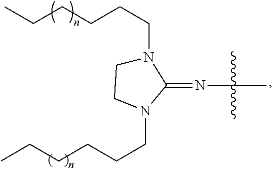

wherein n is 1-20. In some embodiments, —X—R^{L} is selected from:

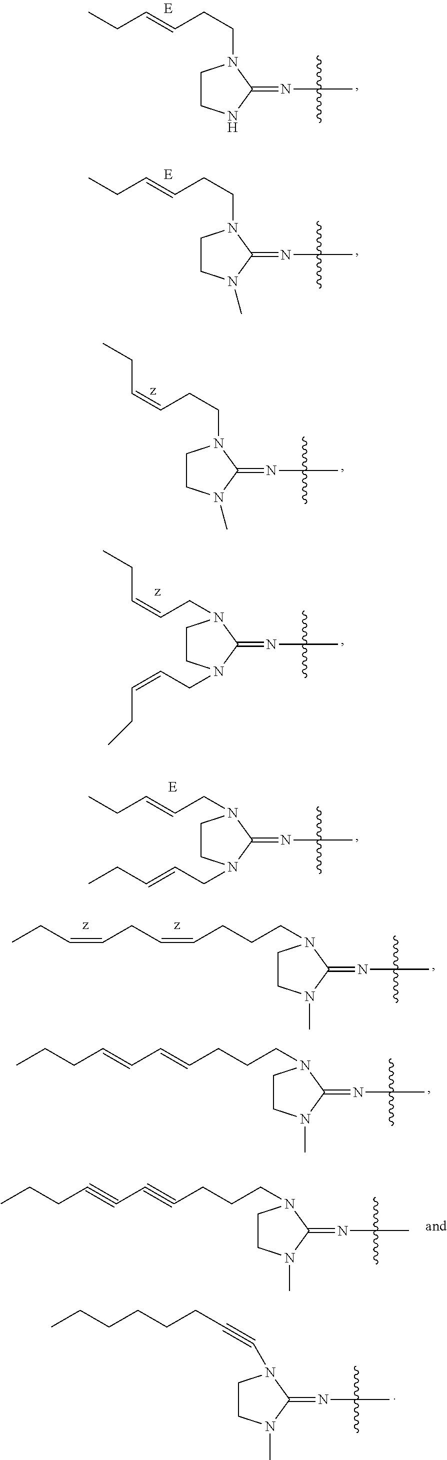

In some embodiments, —X—R^{L} is

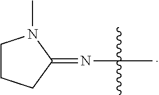 [SMILES: *N=C1CCCN1C]

In some embodiments, —X—R^{L} is

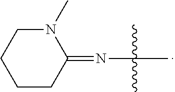 [SMILES: *N=C1CCCCN1C]

In some embodiments, -X—R^{L} is

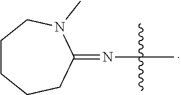 [SMILES: *N=C1CCCCCN1C]

In some embodiments, R^{L} is R″ as described herein. In some embodiments, R^{L} is R as described herein.

In some embodiments, R″ or R^{L} is or comprises an additional chemical moiety. In some embodiments, R″ or R^{L} is or comprises an additional chemical moiety, wherein the additional chemical moiety is or comprises a carbohydrate moiety. In some embodiments, R″ or R^{L} is or comprises a GalNAc. In some embodiments, R^{L} or R″ is replaced with, or is utilized to connect to, an additional chemical moiety.

In some embodiments, X is —O—. In some embodiments, X is -S-. In some embodiments, X is -L^{L}-N(-L^{L}-R^{L})-L^{L}-. In some embodiments, X is —N(-L^{L}-R^{L})-L^{L}-. In some embodiments, X is -L^{L}-N(-L^{L}-R^{L})-. In some embodiments, X is —N(-L^{L}-R^{L})-. In some embodiments, X is -L^{L}-N═C(-L^{L}-R^{L})-L^{L}-In some embodiments, X is —N═C(-L^{L}-R^{L})-L^{L}-. In some embodiments, X is -L^{L}-N═C(-L^{L}-R^{L} In some embodiments, X is —N═C(-L^{L}-R^{L})-. In some embodiments, X is L^{L}. In some embodiments, X is a covalent bond.

In some embodiments, Y is a covalent bond. In some embodiments, Y is —O—. In some embodiments, Y is —N(R′)-. In some embodiments, Z is a covalent bond. In some embodiments, Z is —O—. In some embodiments, Z is —N(R′)-. In some embodiments, R′ is R. In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R. Suitable embodiments for R are described extensively in the present disclosure. As appreciated by those skilled in the art, R embodiments described for a variable that can be R may also be applicable to another variable that can be R. Similarly, embodiments described for a component/moiety (e.g., L) for a variable may also be applicable to other variables that can be or comprise the component/moiety.

In some embodiments, R″ is R′. In some embodiments, R″ is —N(R′)₂.

In some embodiments, —X—R^{L} is —SH. In some embodiments, —X—R^{L} is —OH.

In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of -OP(═O)(-N═C((N(R′)₂)₂—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is -N=C(-L^{L}-R′)₂. In some embodiments, —X—R^{L} is —N═C(-L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁C₆ heteroaliphatic group having 1-5 heteroatoms, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, -P(NR′)—, —P(OR′)[B(R′)₃]-, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L^{L2} is —Cy-. In some embodiments, L^{L1} is a covalent bond. In some embodiments, L^{L3} is a covalent bond. In some embodiments, —X—R^{L} is —N═C(-L^{L1}-Cy-L^{L3}-R′)₂. In some embodiments, —X—R^{L} is

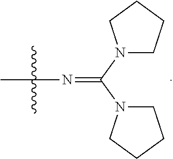 [SMILES: *N=C(N1CCCC1)N1CCCC1]

In some embodiments, —X—R^{L} is

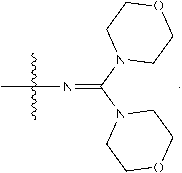 [SMILES: *N=C(N1CCOCC1)N1CCOCC1]

In some embodiments, —X—R^{L} is

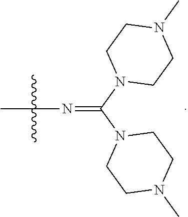 [SMILES: *N=C(N1CCN(C)CC1)N1CCN(C)CC1]

In some embodiments, -X—R^{L} is

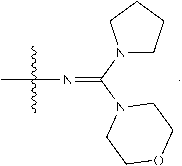 [SMILES: *N=C(N1CCCC1)N1CCOCC1]

In some embodiments, —X—R^{L} is

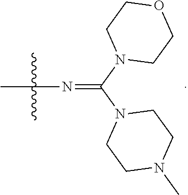 [SMILES: *N=C(N1CCOCC1)N1CCN(C)CC1]

In some embodiments, -X—R^{L} is

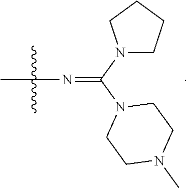 [SMILES: *N=C(N1CCCC1)N1CCN(C)CC1]

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂-, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, -P(NR′)—, —P(OR′)[B(R′)₃]-, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃ ]O—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —C≡C—, —C(R)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C═C—, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, one or more methylene units are optionally and independently replaced by an optionally substituted group selected from —C═C—, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, an internucleotidic linkage is a phosphoryl guanidine internucleotidic linkage. In some embodiments, —X—R^{L} is —N═C[N(R′)₂]₂. In some embodiments, each R′ is independently R. In some embodiments, R is optionally substituted C₁₋₆ aliphatic. In some embodiments, R is methyl. In some embodiments, —X—R^{L} is

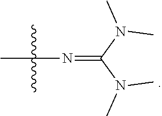 [SMILES: *N=C(N(C)C)N(C)C]

In some embodiments, one R′ on a nitrogen atom is taken with a R′ on the other nitrogen to form a ring as described herein.

In some embodiments, —X—R^{L} is

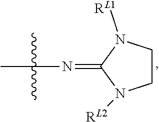

wherein R¹ and R² are independently R′. In some embodiments, —X—R^{L} is

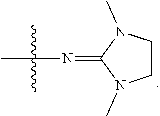 [SMILES: *N=C1N(C)CCN1C]

In some embodiments, —X—R^{L} is

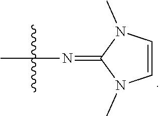 [SMILES: *N=c1n(C)ccn1C]

In some embodiments, two R′ on the same nitrogen are taken together to form a ring as described herein. In some embodiments, -X—R^{L} is

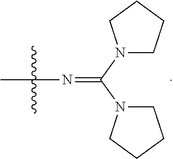 [SMILES: *N=C(N1CCCC1)N1CCCC1]

In some embodiments. —X—R^{L} is

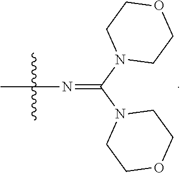 [SMILES: *N=C(N1CCOCC1)N1CCOCC1]

In some embodiments, —X—R^{L} is

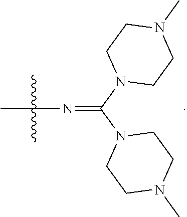 [SMILES: *N=C(N1CCN(C)CC1)N1CCN(C)CC1]

In some embodiments, —X—R^{L} is

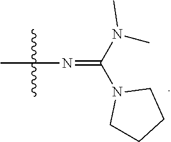 [SMILES: *N=C(N(C)C)N1CCCC1]

In some embodiments, —X—R^{L} is

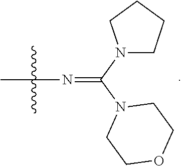 [SMILES: *N=C(N1CCCC1)N1CCOCC1]

In some embodiments, —X—R^{L} is

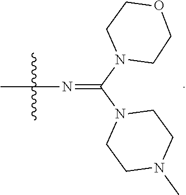 [SMILES: *N=C(N1CCOCC1)N1CCN(C)CC1]

In some embodiments, —X—R^{L} is

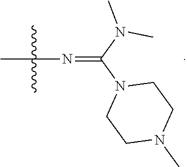 [SMILES: *N=C(N(C)C)N1CCN(C)CC1]

In some embodiments, —X—R^{L} is

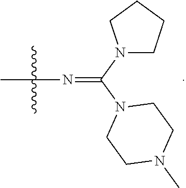 [SMILES: *N=C(N1CCCC1)N1CCN(C)CC1]

In some embodiments, —X—R^{L} is

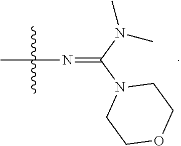 [SMILES: *N=C(N(C)C)N1CCOCC1]

In some embodiments, —X—R^{L} is R as described herein. In some embodiments, R is not hydrogen. In some embodiments, R is optionally substituted C₁₋₆ aliphatic. In some embodiments, R is optionally substituted C₁₋₆ alkyl. In some embodiments, R is methyl.

In some embodiments, —X—R^{L} is selected from Tables below. In some embodiments, X is as described herein. In some embodiments, R^{L} is as described herein. In some embodiments, a linkage has the structure of —Y—P^{L}(—X—R^{L})—Z—, wherein —X—R^{L} is selected from Tables below, and each other variable is independently as described herein. In some embodiments, a linkage has the structure of or comprises —P(O)(—X—R^{L})—, wherein —X—R^{L} is selected from Tables below. In some embodiments, a linkage has the structure of or comprises —P(S)(—X—R^{L})—, wherein —X—R^{L} is selected from Tables below. In some embodiments, a linkage has the structure of or comprises —P(—X—R^{L})—, wherein —X—R^{L} is selected from Tables below. In some embodiments, a linkage has the structure of or comprises —P(O)(—X—R^{L})—O—, wherein —X—R^{L}is selected from Tables below. In some embodiments, a linkage has the structure of or comprises P(S)(—XR^{L})—O—, wherein -X—R^{L} is selected from Tables below. In some embodiments, a linkage has the structure of or comprises —P(—X—R^{L})—O—, wherein —X—R^{L} is selected from Tables below. In some embodiments, a linkage has the structure of -P(O)(—X—R^{L})—O—, wherein —X—R^{L} is selected from Tables below. In some embodiments, a linkage has the structure of -P(S)(—X—R^{L})—O—, wherein -X—R^{L} is selected from Tables below. In some embodiments, a linkage has the structure of -P(—X—R^{L})—O—, wherein —X—R^{L} is selected from Tables below. In some embodiments, P is bonded to a nitrogen atom (e.g., a nitrogen atom in sm01, sml8, etc.). In some embodiments, a linkage has the structure of or comprises —O—P(O)(—X—R^{L})—O—, wherein —X—R^{L} is selected from Tables below. In some embodiments, a linkage has the structure of or comprises —O—P(S)(—X—R^{L})—O—, wherein —X—R^{L} is selected from Tables below. In some embodiments, a linkage has the structure of or comprises —O-P(—X—R^{L})—O—, wherein —X—R^{L} is selected from Tables below. In some embodiments, a linkage has the structure of —O—P(O)(—X—R^{L})—O—, wherein —X—R^{L} is selected from Tables below. In some embodiments, a linkage has the structure of —O—P(S)(—X—R^{L})—O—, wherein —X—R^{L} is selected from Tables below. In some embodiments, a linkage has the structure of —O-P(—X—R^{L})—O—, wherein —X—R^{L} is selected from Tables below. In some embodiments, the Tables below, n is 0-20 or as described herein. As those skilled in the art appreciate, a linkage may exist in a salt form.

Table L-1. Certain useful moieties bonded to linkage phosphorus (e.g., —X—R^{L}).

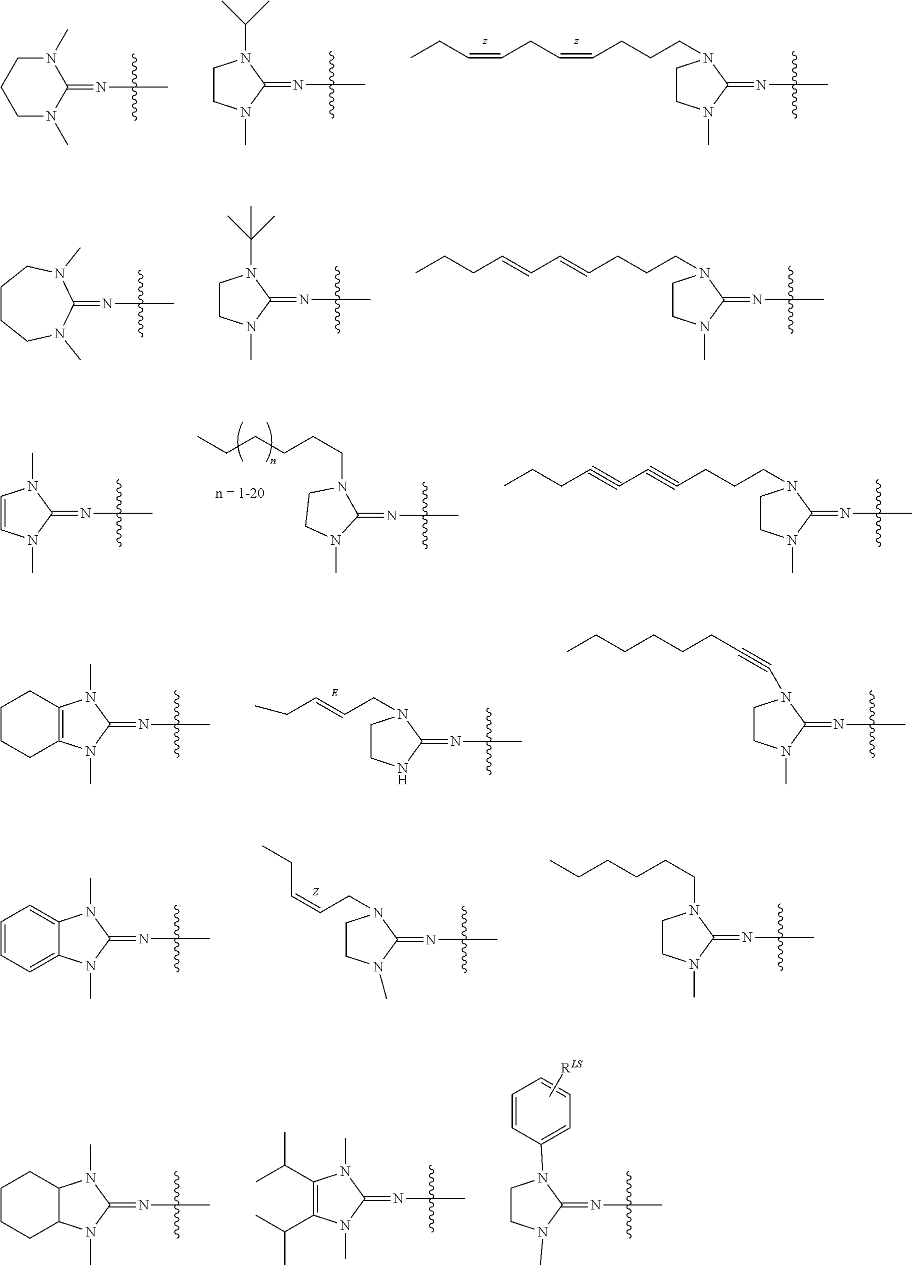

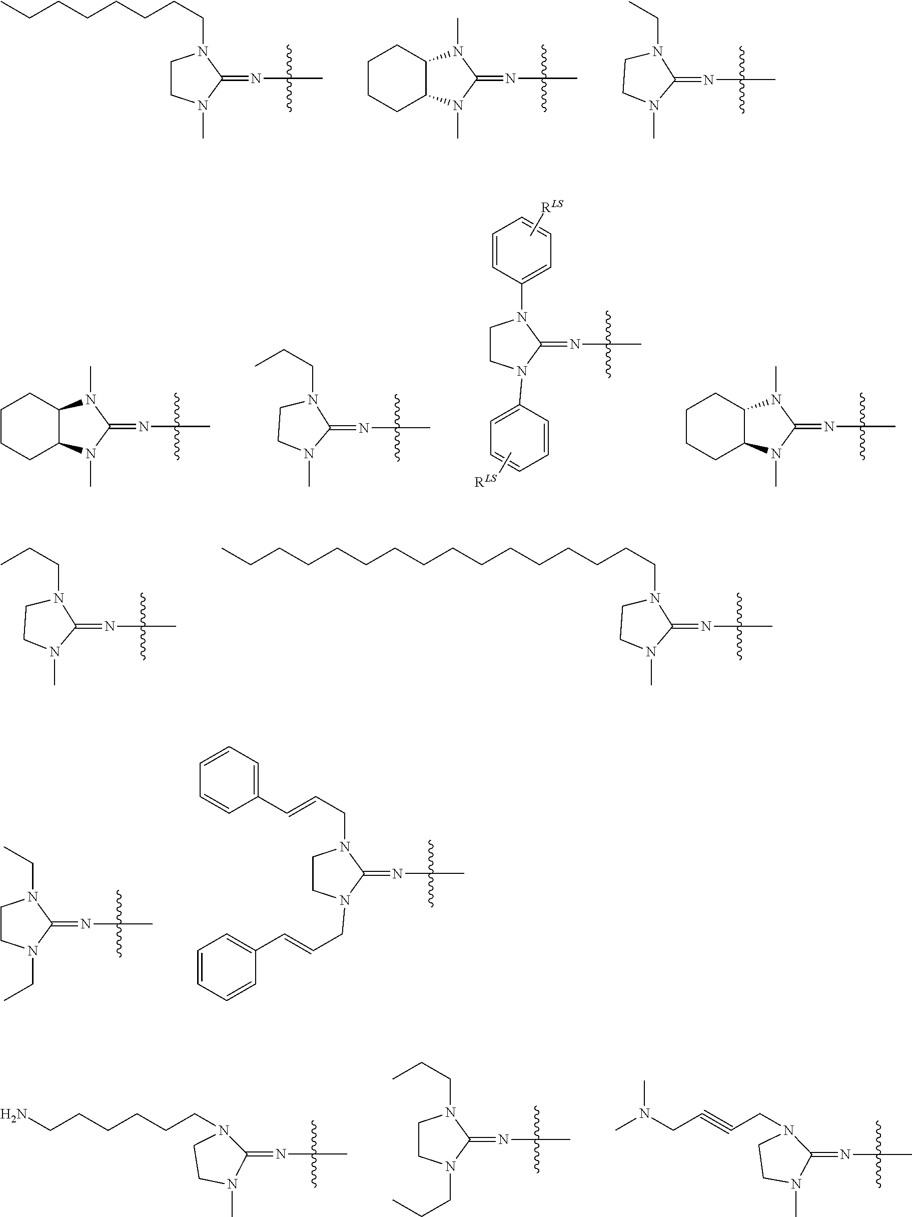

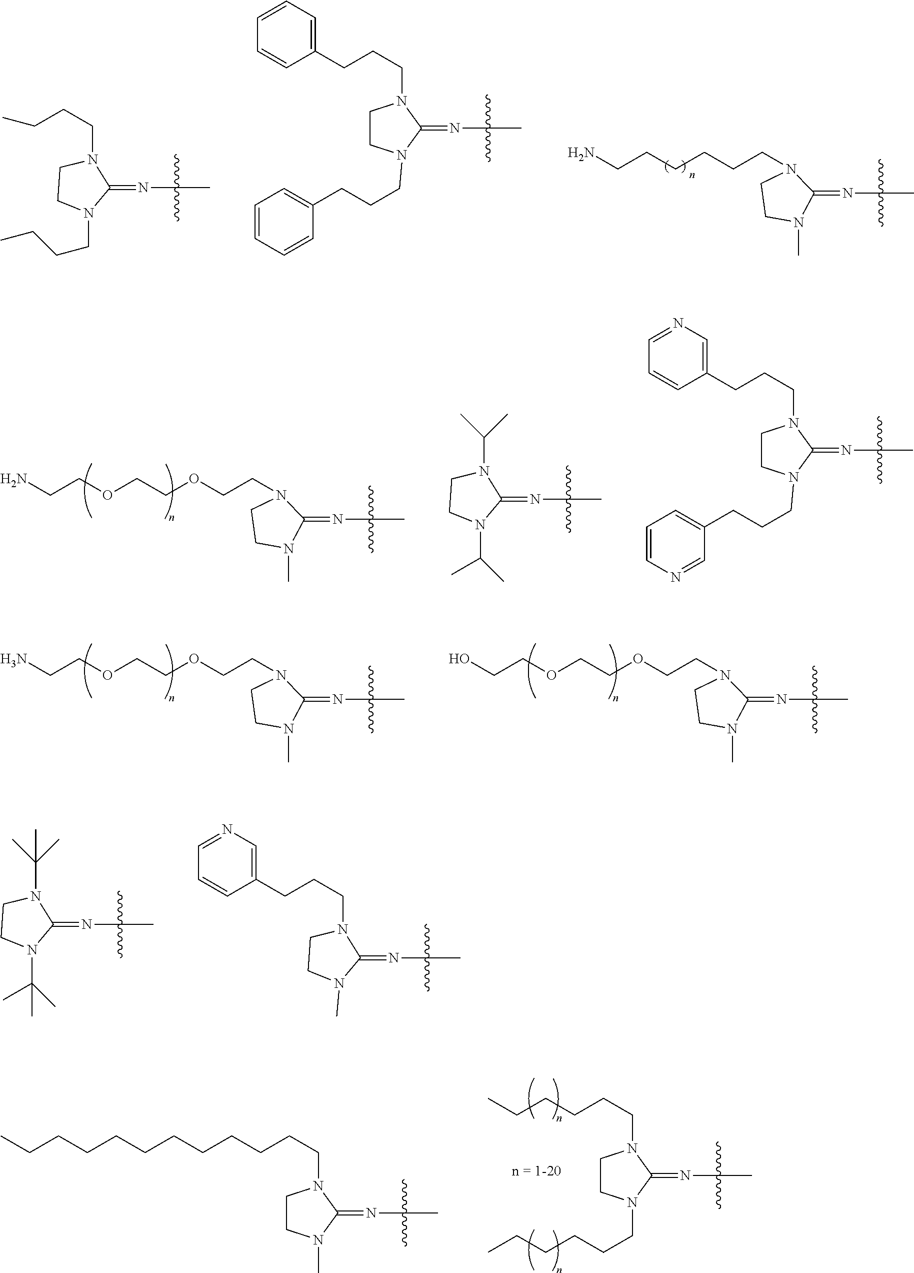

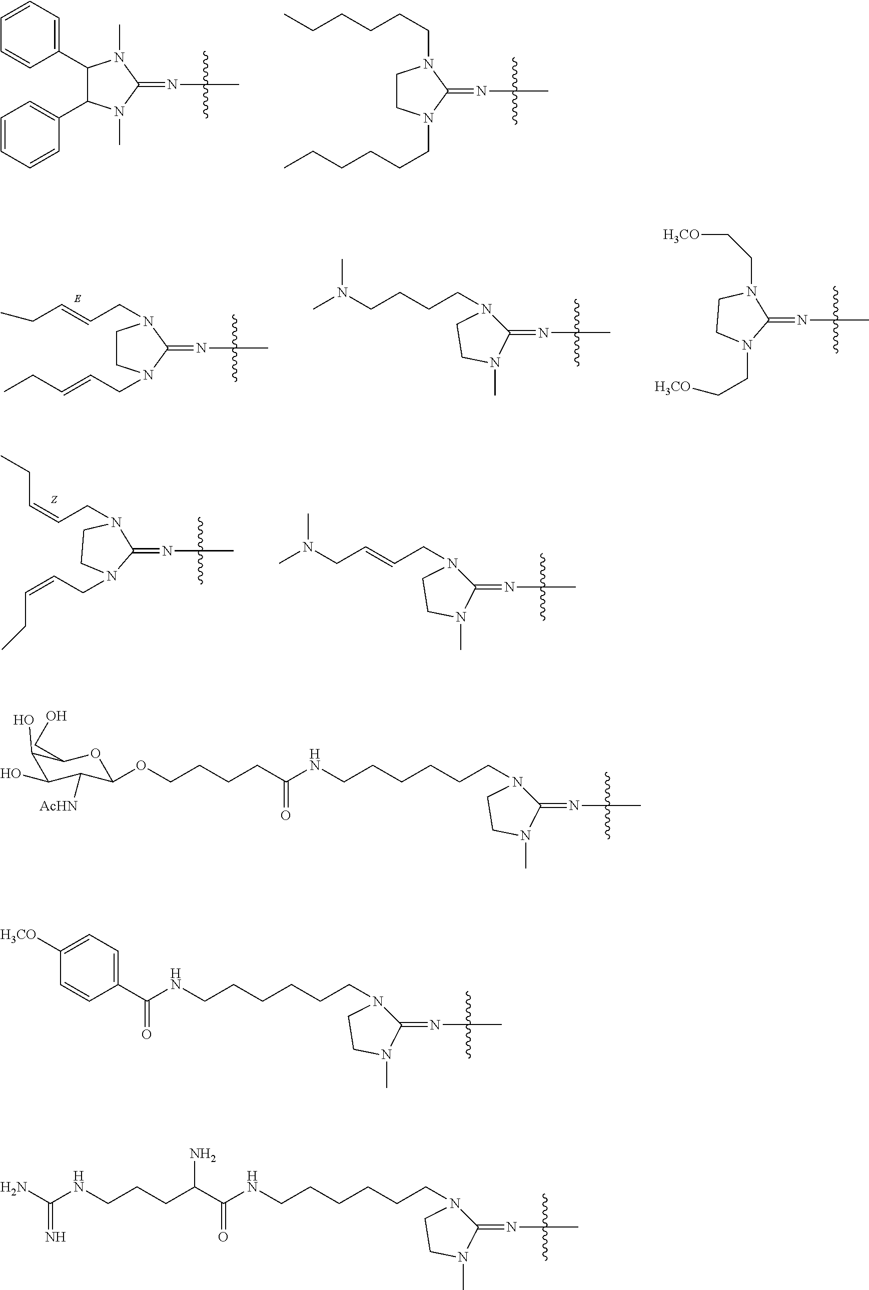

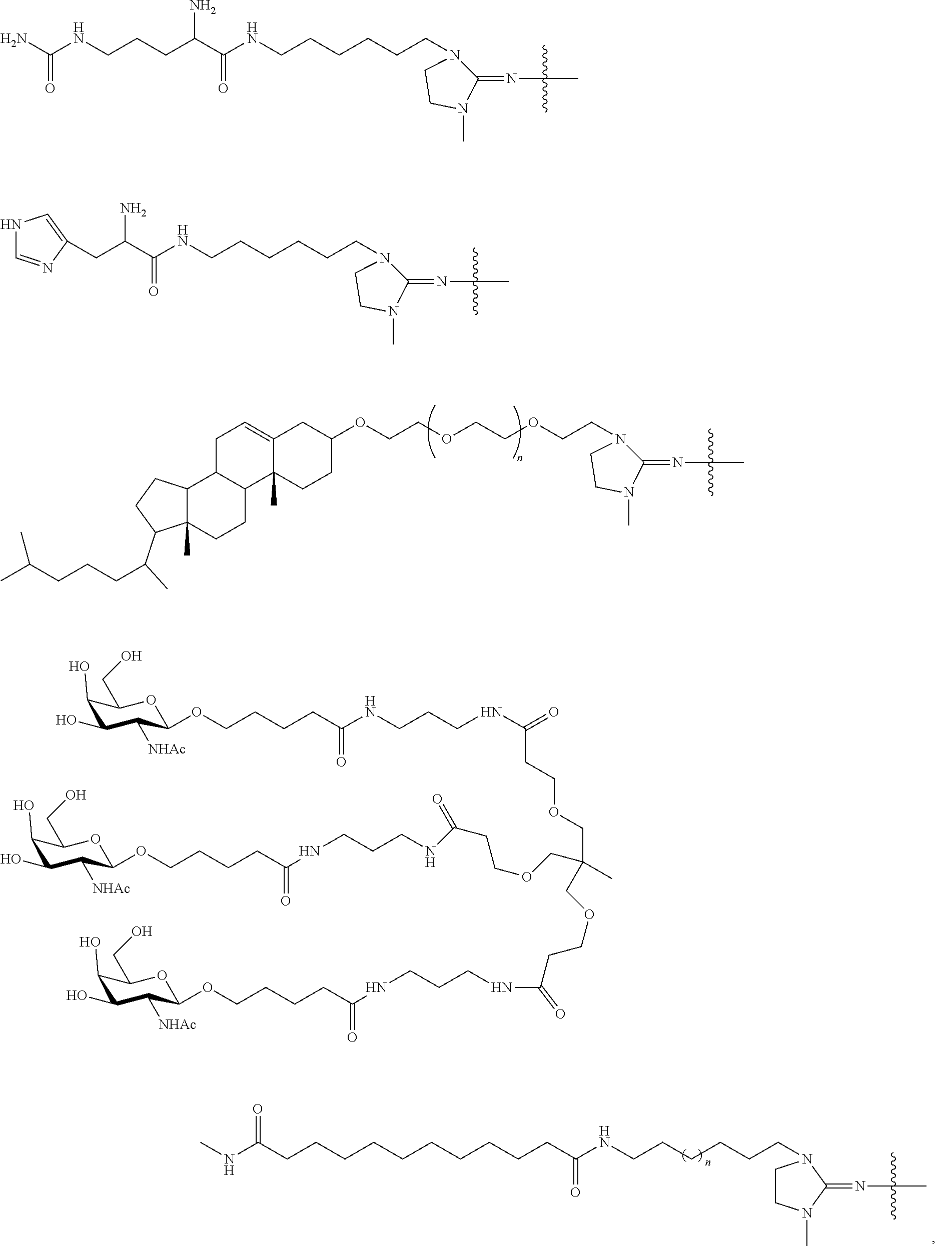

wherein each R^{LS} is independently R^{s}. In some embodiments, each R^{LS} is independently —Cl, —Br, —F, -N(Me)₂, or —NHCOCH₃.

TABLE L-2
Certain useful moieties bonded to linkage phosphorus (e.g., -X-R^{L}).

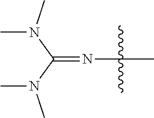 [SMILES: *N=C(N(C)C)N(C)C]

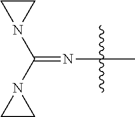 [SMILES: *N=C(N1CC1)N1CC1]

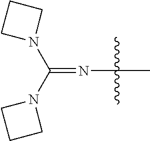 [SMILES: *N=C(N1CCC1)N1CCC1]

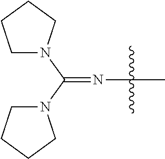 [SMILES: *N=C(N1CCCC1)N1CCCC1]

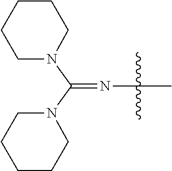 [SMILES: *N=C(N1CCCCC1)N1CCCCC1]

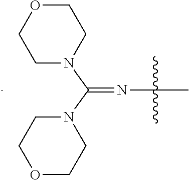 [SMILES: *N=C(N1CCOCC1)N1CCOCC1]

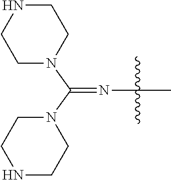 [SMILES: *N=C(N1CCNCC1)N1CCNCC1]

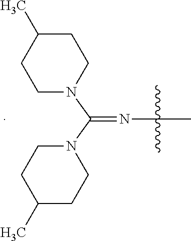 [SMILES: *N=C(N1CCC(C)CC1)N1CCC(C)CC1]

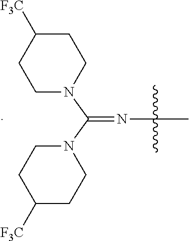 [SMILES: *N=C(N1CCC(C(F)(F)F)CC1)N1CCC(C(F)(F)F)CC1]

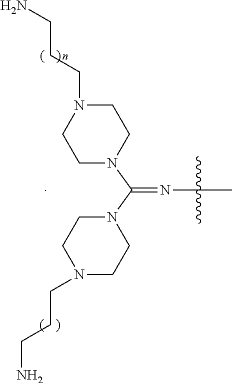

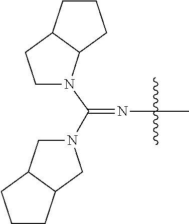 [SMILES: *N=C(N1CC2CCCC2C1)N1CCC2CCCC21]

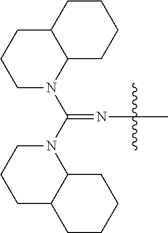 [SMILES: *N=C(N1CCCC2CCCCC21)N1CCCC2CCCCC21]

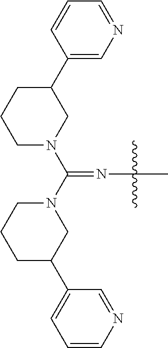 [SMILES: *N=C(N1CCCC(c2cccnc2)C1)N1CCCC(c2cccnc2)C1]

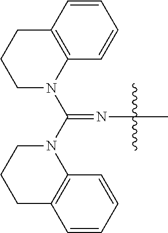 [SMILES: *N=C(N1CCCc2ccccc21)N1CCCc2ccccc21]

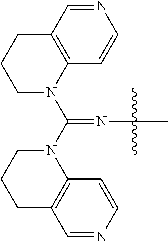 [SMILES: *N=C(N1CCCc2cnccc21)N1CCCc2cnccc21]

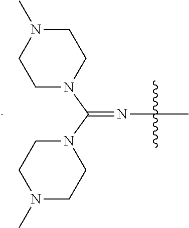 [SMILES: *N=C(N1CCN(C)CC1)N1CCN(C)CC1]

TABLE L-3
Certain useful moieties bonded to linkage phosphorus (e.g., -X-R^{L}).

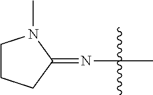 [SMILES: *N=C1CCCN1C]

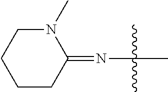 [SMILES: *N=C1CCCCN1C]

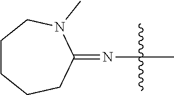 [SMILES: *N=C1CCCCCN1C]

TABLE L-4
Certain useful moieties bonded to linkage phosphorus (e.g., -X-R^{L}).

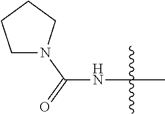 [SMILES: *NC(=O)N1CCCC1]

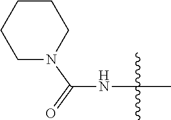 [SMILES: *NC(=O)N1CCCCC1]

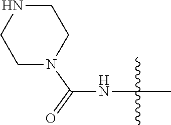 [SMILES: *NC(=O)N1CCNCC1]

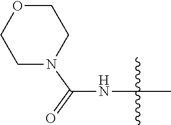 [SMILES: *NC(=O)N1CCOCC1]

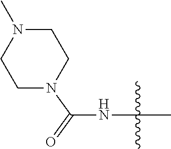 [SMILES: *NC(=O)N1CCN(C)CC1]

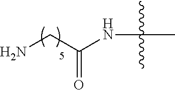

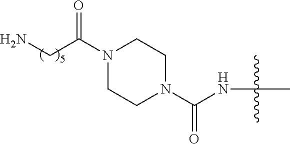

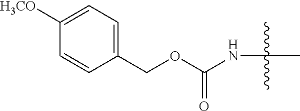 [SMILES: *NC(=O)OCc1ccc(OC)cc1]

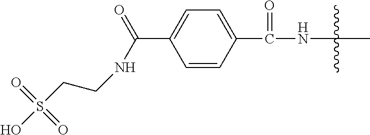 [SMILES: *NC(=O)c1ccc(C(=O)NCCS(=O)(=O)O)cc1]

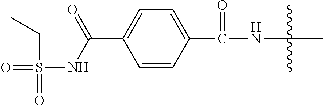 [SMILES: *NC(=O)c1ccc(C(=O)NS(=O)(=O)CC)cc1]

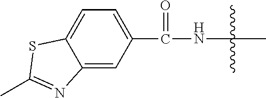 [SMILES: *NC(=O)c1ccc2sc(C)nc2c1]

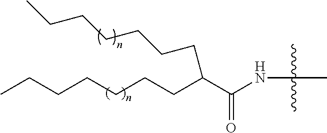

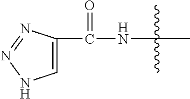 [SMILES: *NC(=O)c1c[nH]nn1]

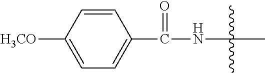 [SMILES: *NC(=O)c1ccc(OC)cc1]

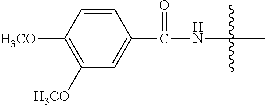 [SMILES: *NC(=O)c1ccc(OC)c(OC)c1]

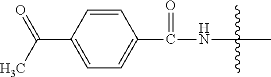 [SMILES: *NC(=O)c1ccc(C(C)=O)cc1]

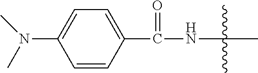 [SMILES: *NC(=O)c1ccc(N(C)C)cc1]

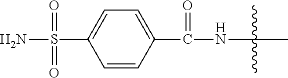 [SMILES: *NC(=O)c1ccc(S(N)(=O)=O)cc1]

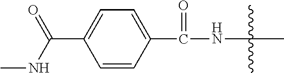 [SMILES: *NC(=O)c1ccc(C(=O)NC)cc1]

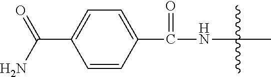 [SMILES: *NC(=O)c1ccc(C(N)=O)cc1]

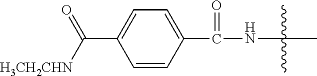

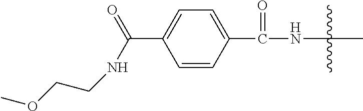 [SMILES: *NC(=O)c1ccc(C(=O)NCCOC)cc1]

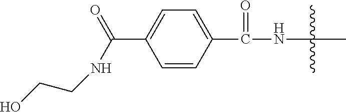 [SMILES: *NC(=O)c1ccc(C(=O)NCCO)cc1]

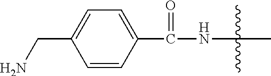 [SMILES: *NC(=O)c1ccc(CN)cc1]

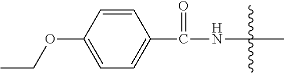 [SMILES: *NC(=O)c1ccc(OCC)cc1]

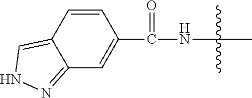 [SMILES: *NC(=O)c1ccc2c[nH]nc2c1]

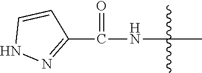 [SMILES: *NC(=O)c1cc[nH]n1]

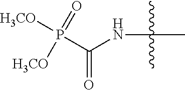 [SMILES: *NC(=O)P(=O)(OC)OC]

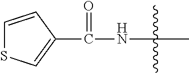 [SMILES: *NC(=O)c1ccsc1]

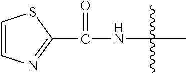 [SMILES: *NC(=O)c1nccs1]

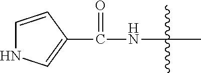 [SMILES: *NC(=O)c1cc[nH]c1]

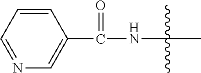 [SMILES: *NC(=O)c1cccnc1]

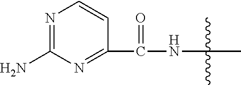 [SMILES: *NC(=O)c1ccnc(N)n1]

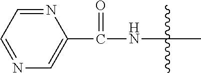 [SMILES: *NC(=O)c1cnccn1]

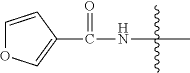 [SMILES: *NC(=O)c1ccoc1]

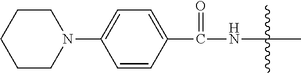 [SMILES: *NC(=O)c1ccc(N2CCCCC2)cc1]

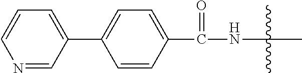 [SMILES: *NC(=O)c1ccc(-c2cccnc2)cc1]

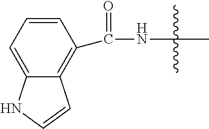 [SMILES: *NC(=O)c1cccc2[nH]ccc12]

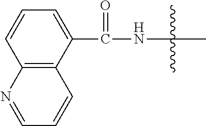 [SMILES: *NC(=O)c1cccc2ncccc12]

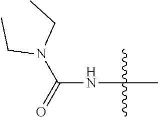 [SMILES: *NC(=O)N(CC)CC]

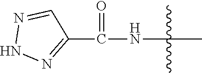 [SMILES: *NC(=O)c1cn[nH]n1]

TABLE L-5
Certain useful moieties bonded to linkage phosphorus (e.g., -X-R^{L}).

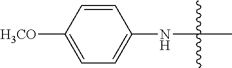 [SMILES: *Nc1ccc(OC)cc1]

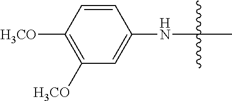 [SMILES: *Nc1ccc(OC)c(OC)c1]

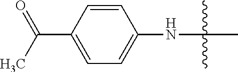 [SMILES: *Nc1ccc(C(C)=O)cc1]

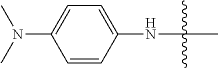 [SMILES: *Nc1ccc(N(C)C)cc1]

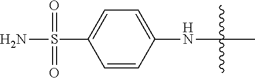 [SMILES: *Nc1ccc(S(N)(=O)=O)cc1]

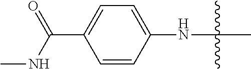 [SMILES: *Nc1ccc(C(=O)NC)cc1]

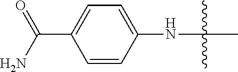 [SMILES: *Nc1ccc(C(N)=O)cc1]

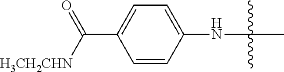

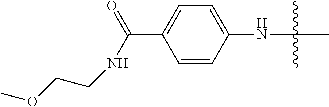 [SMILES: *Nc1ccc(C(=O)NCCOC)cc1]

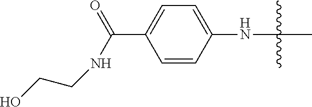 [SMILES: *Nc1ccc(C(=O)NCCO)cc1]

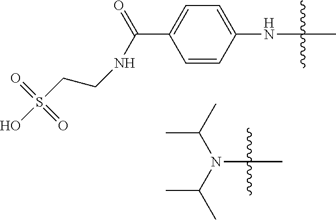

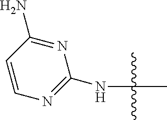 [SMILES: *Nc1nccc(N)n1]

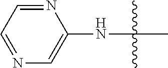 [SMILES: *Nc1cnccn1]

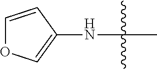 [SMILES: *Nc1ccoc1]

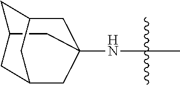 [SMILES: *NC12CC3CC(CC(C3)C1)C2]

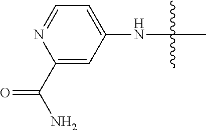 [SMILES: *Nc1ccnc(C(N)=O)c1]

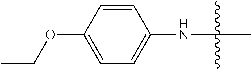 [SMILES: *Nc1ccc(OCC)cc1]

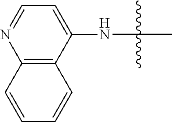 [SMILES: *Nc1ccnc2ccccc12]

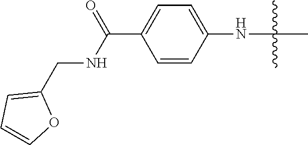 [SMILES: *Nc1ccc(C(=O)NCc2ccco2)cc1]

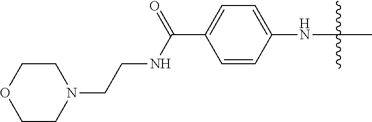 [SMILES: *Nc1ccc(C(=O)NCCN2CCOCC2)cc1]

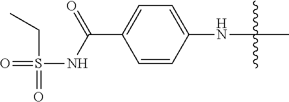 [SMILES: *Nc1ccc(C(=O)NS(=O)(=O)CC)cc1]

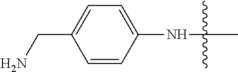 [SMILES: *Nc1ccc(CN)cc1]

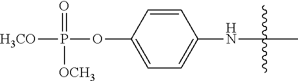 [SMILES: *Nc1ccc(OP(=O)(OC)OC)cc1]

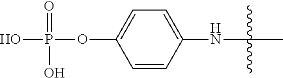 [SMILES: *Nc1ccc(OP(=O)(O)O)cc1]

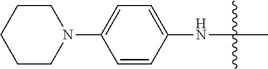 [SMILES: *Nc1ccc(N2CCCCC2)cc1]

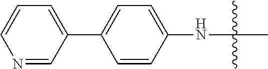 [SMILES: *Nc1ccc(-c2cccnc2)cc1]

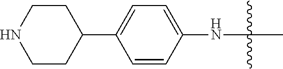 [SMILES: *Nc1ccc(C2CCNCC2)cc1]

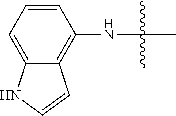 [SMILES: *Nc1cccc2[nH]ccc12]

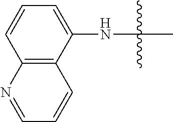 [SMILES: *Nc1cccc2ncccc12]

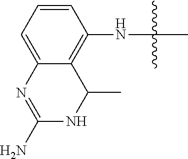 [SMILES: *Nc1cccc2c1C(C)NC(N)=N2]

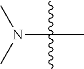 [SMILES: *N(C)C]

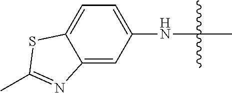 [SMILES: *Nc1ccc2sc(C)nc2c1]

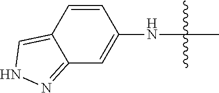 [SMILES: *Nc1ccc2c[nH]nc2c1]

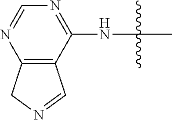 [SMILES: *Nc1ncnc2c1C=NC2]

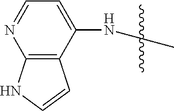 [SMILES: *Nc1ccnc2[nH]ccc12]

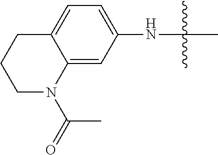 [SMILES: *Nc1ccc2c(c1)N(C(C)=O)CCC2]

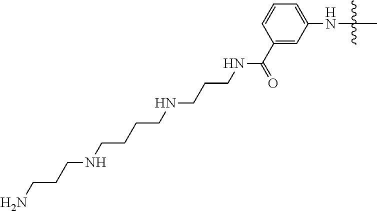 [SMILES: *Nc1cccc(C(=O)NCCCNCCCCNCCCN)c1]

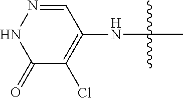 [SMILES: *Nc1cn[nH]c(=O)c1Cl]

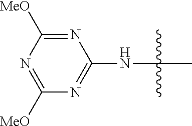 [SMILES: *Nc1nc(OC)nc(OC)n1]

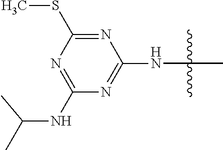 [SMILES: *Nc1nc(NC(C)C)nc(SC)n1]

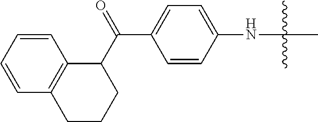 [SMILES: *Nc1ccc(C(=O)C2CCCc3ccccc32)cc1]

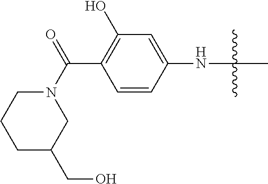 [SMILES: *Nc1ccc(C(=O)N2CCCC(CO)C2)c(O)c1]

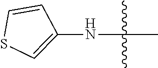 [SMILES: *Nc1ccsc1]

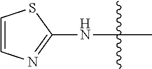 [SMILES: *Nc1nccs1]

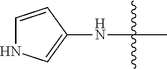 [SMILES: *Nc1cc[nH]c1]

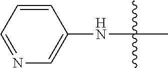 [SMILES: *Nc1cccnc1]

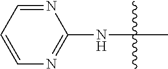 [SMILES: *Nc1ncccn1]

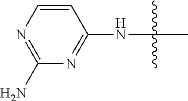 [SMILES: *Nc1ccnc(N)n1]

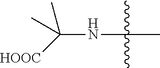 [SMILES: *NC(C)(C)C(=O)O]

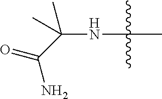 [SMILES: *NC(C)(C)C(N)=O]

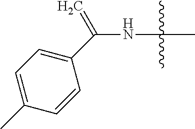 [SMILES: *NC(=C)c1ccc(C)cc1]

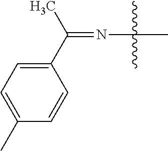 [SMILES: *N=C(C)c1ccc(C)cc1]

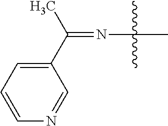 [SMILES: *N=C(C)c1cccnc1]

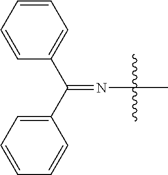 [SMILES: *N=C(c1ccccc1)c1ccccc1]

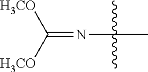 [SMILES: *N=C(OC)OC]

TABLE L-6
Certain useful moieties bonded to linkage phosphorus (e.g., -X-R^{L}).

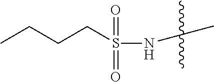 [SMILES: *NS(=O)(=O)CCCC]

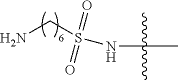

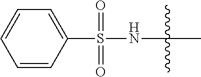 [SMILES: *NS(=O)(=O)c1ccccc1]

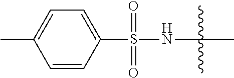 [SMILES: *NS(=O)(=O)c1ccc(C)cc1]

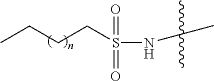

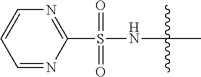 [SMILES: *NS(=O)(=O)c1ncccn1]

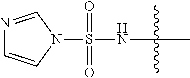 [SMILES: *NS(=O)(=O)n1ccnc1]

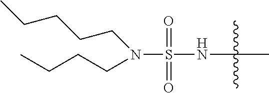 [SMILES: *NS(=O)(=O)N(CCCC)CCCCC]

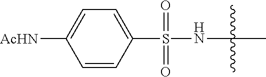 [SMILES: *NS(=O)(=O)c1ccc(NC(C)=O)cc1]

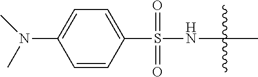 [SMILES: *NS(=O)(=O)c1ccc(N(C)C)cc1]

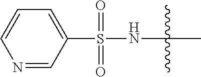 [SMILES: *NS(=O)(=O)c1cccnc1]

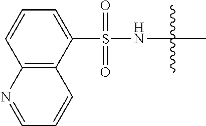 [SMILES: *NS(=O)(=O)c1cccc2ncccc12]

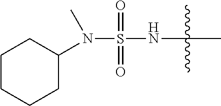 [SMILES: *NS(=O)(=O)N(C)C1CCCCC1]

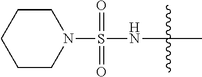 [SMILES: *NS(=O)(=O)N1CCCCC1]

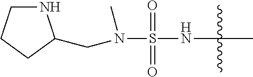 [SMILES: *NS(=O)(=O)N(C)CC1CCCN1]

In some embodiments, an internucleotidic linkage, e.g., an non-negatively charged internucleotidic linkage or a neutral internucleotidic linkage, has the structure of -L^{L1}-Cy^{IL}-L^{L2}-. In some embodiments, L^{L1} is bonded to a 3′-carbon of a sugar. In some embodiments, L^{L2} is bonded to a 5′-carbon of a sugar. In some embodiments, L^{L1} is —O—CH₂—. In some embodiments, L^{L2} is a covalent bond. In some embodiments, L^{L2} is a —N(R′)-. In some embodiments, L^{L2} is a —NH—. In some embodiments, L^{L2} is bonded to a 5′-carbon of a sugar, which 5′-carbon is substituted with ═O. In some embodiments, Cy^{IL} is optionally substituted 3-10 membered saturated, partially unsaturated, or aromatic ring having 0-5 heteroatoms. In some embodiments, Cy^{IL} is an optionally substituted triazole ring. In some embodiments, Cy^{IL} is

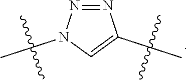 [SMILES: *c1cn(*)nn1]

In some embodiments, a linkage is

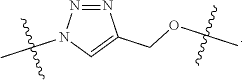 [SMILES: *OCc1cn(*)nn1]

In some embodiments, a non-negatively charged internucleotidic linkage has the structure of -OP(=W)(-N(R′)₂)-O—.

In some embodiments, R′ is R. In some embodiments, R′ is —H. In some embodiments, R′ is —C(O)R. In some embodiments, R′ is —C(O)OR. In some embodiments, R′ is —S(O)₂R.

In some embodiments, R″ is —NHR′. In some embodiments, —N(R′)₂ is —NHR′.

As described herein, some embodiments, R is —H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, R′ is or comprises optionally substituted triazolyl. In some embodiments, a modified internucleotidic linkage (e.g., a non-negatively charged internucleotidic linkage) comprises optionally substituted alkynyl. In some embodiments, R′ is optionally substituted alkynyl. In some embodiments, R′ comprises an optionally substituted triple bond. In some embodiments, a modified internucleotidic linkage comprises a triazole or alkyne moiety. In some embodiments, R′ is or comprises an optionally substituted triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted guanidine moiety. In some embodiments, a modified internucleotidic linkage comprises an optionally substituted cyclic guanidine moiety. In some embodiments, R′, R^{L}, or —X—R^{L}, is or comprises an optionally substituted guanidine moiety. In some embodiments, R′, R^{L}, or —X—R^{L}, is or comprises an optionally substituted cyclic guanidine moiety. In some embodiments, R′, R^{L}, or —X—R^{L} comprises an optionally substituted cyclic guanidine moiety and an internucleotidic linkage has the structure of:

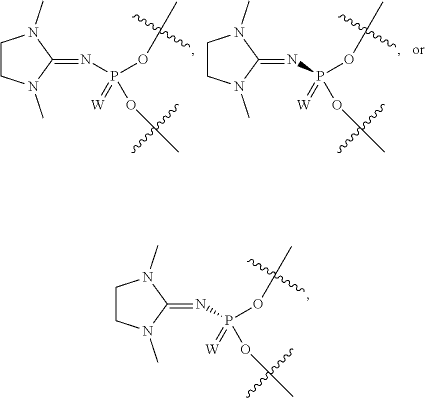

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

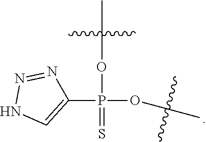 [SMILES: *OP(=S)(O*)c1c[nH]nn1]

In some embodiments, an internucleotidic linkage comprising a triazole moiety has the structure of

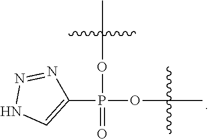 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

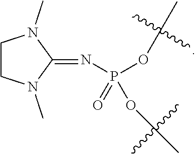 [SMILES: *OP(=O)(N=C1N(C)CCN1C)O*]

In some embodiments, a non-negatively charged internucleotidic linkage, or a neutral internucleotidic linkage, is or comprising a structure selected from

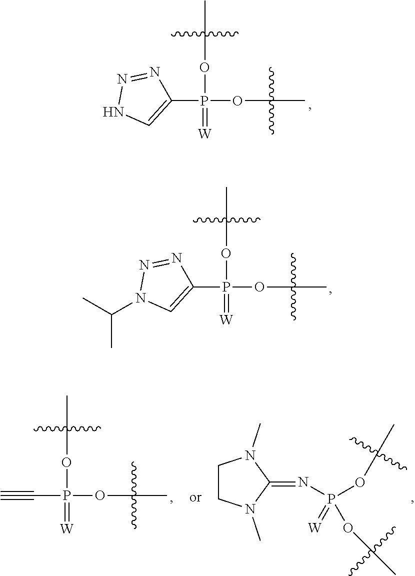

wherein W is O or S.

In some embodiments, an internucleotidic linkage comprises a Tmg group

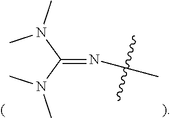 [SMILES: *N=C(N(C)C)N(C)C]

In some embodiments, an internucleotidic linkage comprises a Tmg group and has the structure of

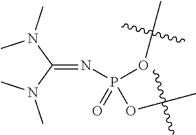 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker. e.g., ═N— when the heterocyclyl group is part of a guanidine moiety who directed bonded to a linkage phosphorus through its ═N—. In some embodiments, a non-negatively charged internucleotidic linkage comprises an optionally substituted

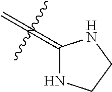 [SMILES: *=C1NCCN1]

group. In some embodiments, a non-negatively charged internucleotidic linkage comprises an substituted

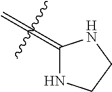 [SMILES: *=C1NCCN1]

group. In some embodiments, a non-negatively charged internucleotidic linkage comprises a

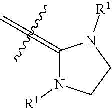

group. In some embodiments, each R¹ is independently optionally substituted C₁₋₆ alkyl. In some embodiments, each R¹ is independently methyl.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an internucleotidic linkage comprises no linkage phosphorus. In some embodiments, an internucleotidic linkage has the structure of —C(O)-(O)- or —C(O)—N(R′)—, wherein R′ is as described herein. In some embodiments, an internucleotidic linkage has the structure of —C(O)-(O)—. In some embodiments, an internucleotidic linkage has the structure of —C(O)-N(R′)—, wherein R′ is as described herein. In various embodiments, —C(O)— is bonded to nitrogen. In some embodiments, an internucleotidic linkage is or comprises —C(O)—O- which is part of a carbamate moiety. In some embodiments, an internucleotidic linkage is or comprises —C(O)—O- which is part of a urea moiety.

In some embodiments, an oligonucleotide comprises 1-20, 1-15, 1-10, 1-5, or 1, 2, 3, 4, 5, 6, 7, 8, 9, 10, or more non-negatively charged internucleotidic linkages. In some embodiments, an oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

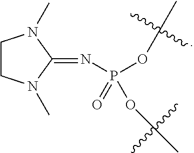 [SMILES: *OP(=O)(N=C1N(C)CCN1C)O*]

In some embodiments, an oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is

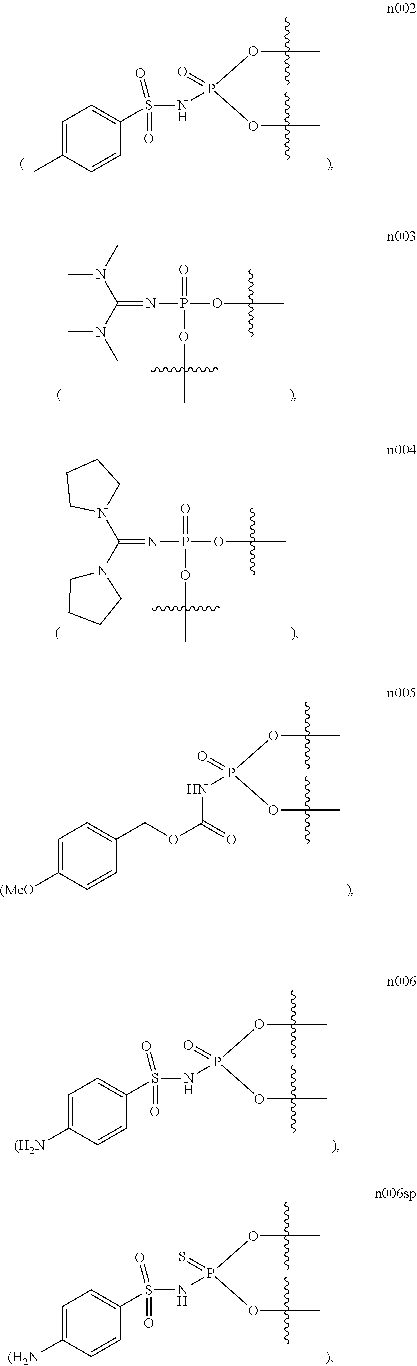

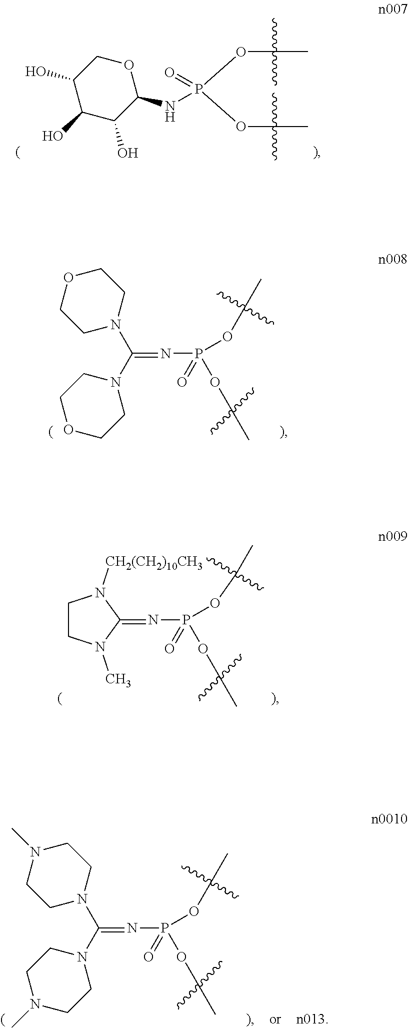

In some embodiments, a non-negatively charged internucleotide linkage is

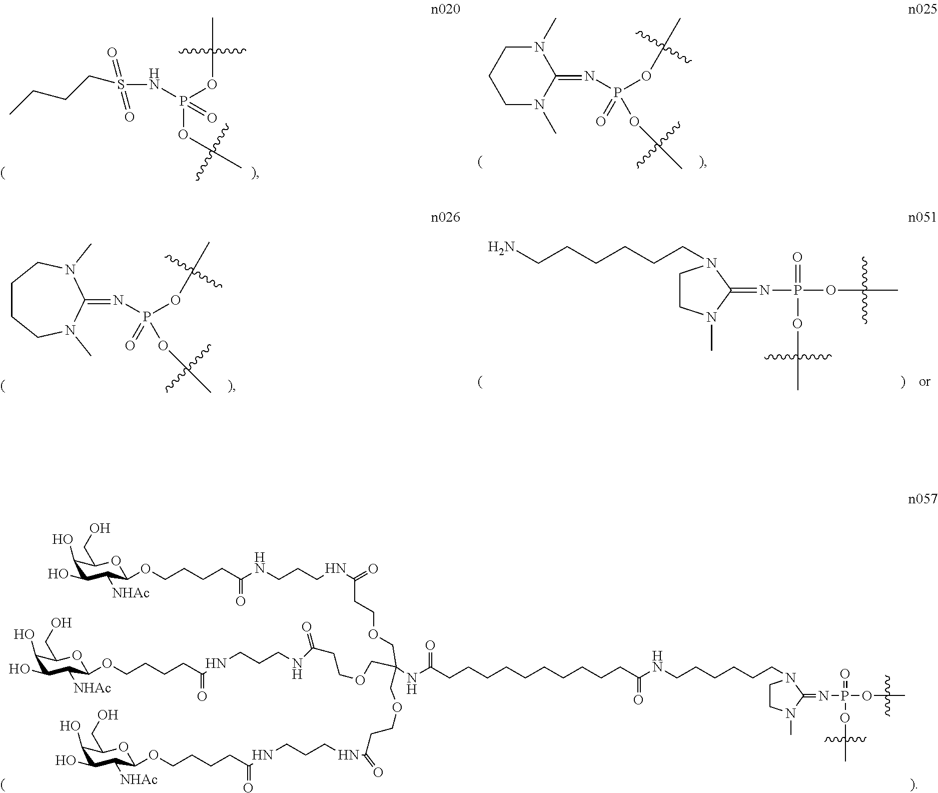

In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, internucleotidic linkages connect sugars that are not ribose sugars, e.g., sugars comprising N ring atoms and acyclic sugars as described herein.

In some embodiments, each nucleoside units connected by an internucleotidic linkage independently comprises a nucleobase which is independently an optionally substituted A, T, C, G, or U. or an optionally substituted tautomer of A, T, C, G or U.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394,333, 9,744,183, 9,605,019, 9,598,458, 9,982,257, U.S. Ser. Nos. 10/160,969, 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.,) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, and/or WO 2019/032612, such internucleotidic linkages of each of which are independently incorporated herein by reference.

As described herein, various variables can be R, e.g., R′, R^{L}, etc. Various embodiments for R are described in the present disclosure (e.g., when describing variables that can be R). Such embodiments are generally useful for all variables that can be R. In some embodiments, R is hydrogen. In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) aliphatic. In some embodiments, R is optionally substituted C₁₋₂₀ aliphatic. In some embodiments, R is optionally substituted C₁₋₁₀ aliphatic. In some embodiments, R is optionally substituted C₁₋₆ aliphatic. In some embodiments, R is optionally substituted alkyl. In some embodiments, R is optionally substituted C₁₋₆ alkyl. In some embodiments, R is optionally substituted methyl. In some embodiments, R is methyl. In some embodiments, R is optionally substituted ethyl. In some embodiments, R is optionally substituted propyl. In some embodiments, R is isopropyl. In some embodiments, R is optionally substituted butyl. In some embodiments, R is optionally substituted pentyl. In some embodiments, R is optionally substituted hexyl.

In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) cycloaliphatic. In some embodiments, R is optionally substituted cycloalkyl. In some embodiments, cycloaliphatic is monocyclic, bicyclic, or polycyclic, wherein each monocyclic unit is independently saturated or partially saturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted adamantyl.

In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) heteroaliphatic having 1-10 heteroatoms. In some embodiments, R is optionally substituted C₁₋₂₀ aliphatic having 1-10 heteroatoms. In some embodiments, R is optionally substituted C₁₋₁₀ aliphatic having 1-10 heteroatoms. In some embodiments, R is optionally substituted C₁₋₆ aliphatic having 1-3 heteroatoms. In some embodiments, R is optionally substituted heteroalkyl. In some embodiments, R is optionally substituted C₁₋₆ heteroalkyl. In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) heterocycloaliphatic having 1-10 heteroatoms. In some embodiments, R is optionally substituted heteroclycloalkyl. In some embodiments, heterocycloaliphatic is monocyclic, bicyclic, or polycyclic, wherein each monocyclic unit is independently saturated or partially saturated.

In some embodiments, R is optionally substituted C₆₋₃₀ aryl. In some embodiments, R is optionally substituted phenyl. In some embodiments, R is optionally substituted phenyl. In some embodiments, R is C₆₋₁₄ aryl. In some embodiments, R is optionally substituted bicyclic aryl. In some embodiments, R is optionally substituted polycyclic aryl. In some embodiments, R is optionally substituted C₆₋₃₀ arylaliphatic. In some embodiments, R is C₆₋₃₀ arylheteroaliphatic having 1-10 heteroatoms.

In some embodiments, R is optionally substituted 5-30 (5, 6, 7, 8, 9, 10, 11, 12, 13, 14, 15, 16, 17, 18, 19, 20, 21, 22, 23, 24, 25, 26, 27, 28, 29 or 30) membered heteroaryl having 1-10 heteroatoms. In some embodiments, R is optionally substituted 5-20 membered heteroaryl having 1-10 heteroatoms. In some embodiments, R is optionally substituted 5-10 membered heteroaryl having 1-10 heteroatoms. In some embodiments, R is optionally substituted 5-membered heteroaryl having 1-5 heteroatoms. In some embodiments, R is optionally substituted 5-membered heteroaryl having 1-4 heteroatoms. In some embodiments, R is optionally substituted 5-membered heteroaryl having 1-3 heteroatoms. In some embodiments, R is optionally substituted 5-membered heteroaryl having 1-2 heteroatoms. In some embodiments, R is optionally substituted 5-membered heteroaryl having one heteroatom. In some embodiments, R is optionally substituted 6-membered heteroaryl having 1-5 heteroatoms. In some embodiments, R is optionally substituted 6-membered heteroaryl having 1-4 heteroatoms. In some embodiments, R is optionally substituted 6-membered heteroaryl having 1-3 heteroatoms. In some embodiments, R is optionally substituted 6-membered heteroaryl having 1-2 heteroatoms. In some embodiments, R is optionally substituted 6-membered heteroaryl having one heteroatom. In some embodiments, R is optionally substituted monocyclic heteroaryl. In some embodiments, R is optionally substituted bicyclic heteroaryl. In some embodiments, R is optionally substituted polycyclic heteroaryl. In some embodiments, a heteroatom is nitrogen.

In some embodiments, R is optionally substituted 2-pyridinyl. In some embodiments, R is optionally substituted 3-pyridinyl. In some embodiments, R is optionally substituted 4-pyridinyl. In some embodiments, R is optionally substituted

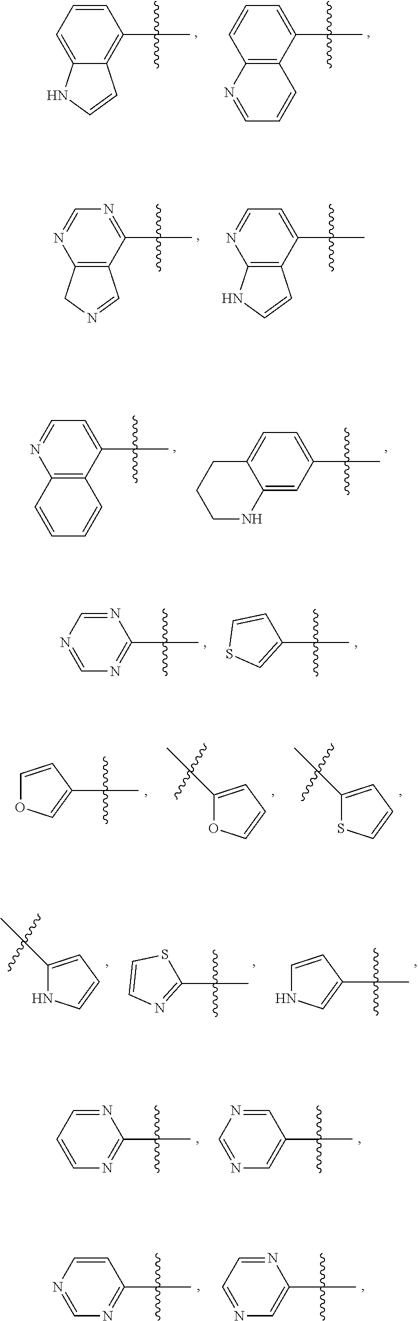

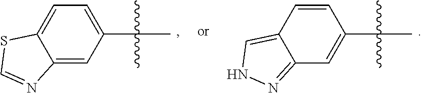 [SMILES: *c1ccc2scnc2c1 ; *c1ccc2c[nH]nc2c1]

In some embodiments, R is optionally substituted 3-30 (3, 4, 5. 6, 7, 8, 9, 10, 11, 12, 13, 14, 15, 16, 17, 18, 19, 20, 21, 22, 23, 24, 25, 26, 27, 28, 29 or 30) membered heterocyclyl having 1-10 heteroatoms. In some embodiments, R is optionally substituted 3-membered heterocyclyl having 1-2 heteroatoms. In some embodiments, R is optionally substituted 4-membered heterocyclyl having 1-2 heteroatoms. In some embodiments, R is optionally substituted 5-20 membered heterocyclyl having 1-10 heteroatoms. In some embodiments, R is optionally substituted 5-10 membered heterocyclyl having 1-10 heteroatoms. In some embodiments, R is optionally substituted 5-membered heterocyclyl having 1-5 heteroatoms. In some embodiments, R is optionally substituted 5-membered heterocyclyl having 1-4 heteroatoms. In some embodiments, R is optionally substituted 5-membered heterocyclyl having 1-3 heteroatoms. In some embodiments, R is optionally substituted 5-membered heterocyclyl having 1-2 heteroatoms. In some embodiments, R is optionally substituted 5-membered heterocyclyl having one heteroatom. In some embodiments, R is optionally substituted 6-membered heterocyclyl having 1-5 heteroatoms. In some embodiments, R is optionally substituted 6-membered heterocyclyl having 1-4 heteroatoms. In some embodiments, R is optionally substituted 6-membered heterocyclyl having 1-3 heteroatoms. In some embodiments, R is optionally substituted 6-membered heterocyclyl having 1-2 heteroatoms. In some embodiments, R is optionally substituted 6-membered heterocyclyl having one heteroatom. In some embodiments, R is optionally substituted monocyclic heterocyclyl. In some embodiments, R is optionally substituted bicyclic heterocyclyl. In some embodiments, R is optionally substituted polycyclic heterocyclyl. In some embodiments, R is optionally substituted saturated heterocyclyl. In some embodiments, R is optionally substituted partially unsaturated heterocyclyl. In some embodiments, a heteroatom is nitrogen. In some embodiments, R is optionally substituted

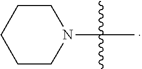 [SMILES: *N1CCCCC1]

In some embodiments, R is optionally substituted

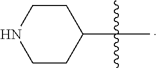 [SMILES: *C1CCNCC1]

In some embodiments, R is optionally substituted

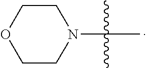 [SMILES: *N1CCOCC1]

In some embodiments, two R groups are optionally and independently taken together to form a covalent bond. In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

Various variables may comprises an optionally substituted ring, or can be taken together with their intervening atom(s) to form a ring. In some embodiments, a ring is 3-30 (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) membered. In some embodiments, a ring is 3-20 membered. In some embodiments, a ring is 3-15 membered. In some embodiments, a ring is 3-10 membered. In some embodiments, a ring is 3-8 membered. In some embodiments, a ring is 3-7 membered. In some embodiments, a ring is 3-6 membered. In some embodiments, a ring is 4-20 membered. In some embodiments, a ring is 5-20 membered. In some embodiments, a ring is monocyclic. In some embodiments, a ring is bicyclic. In some embodiments, a ring is polycyclic. In some embodiments, each monocyclic ring or each monocyclic ring unit in bicyclic or polycyclic rings is independently saturated, partially saturated or aromatic. In some embodiments, each monocyclic ring or each monocyclic ring unit in bicyclic or polycyclic rings is independently 3-10 membered and has 0-5 heteroatoms.

In some embodiments, each heteroatom is independently selected oxygen, nitrogen, sulfur, silicon, and phosphorus. In some embodiments, each heteroatom is independently selected oxygen, nitrogen, sulfur, and phosphorus. In some embodiments, each heteroatom is independently selected oxygen, nitrogen, and sulfur. In some embodiments, a heteroatom is in an oxidized form.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobases, sugars, internucleotidic linkages, chiral auxiliaries/reagents, and technologies for oligonucleotide synthesis (reagents, conditions, cycles, etc.) of each of which is independently incorporated herein by reference.

In some embodiments, each internucleotidic linkage in an oligonucleotide is independently selected from a natural phosphate linkage, a phosphorothioate linkage, and a non-negatively charged internucleotidic linkage (e.g., n001, n002, n003, n004, n005, n006, n007, n008, n009, n010, n013, etc.). In some embodiments, each internucleotidic linkage in an oligonucleotide is independently selected from a natural phosphate linkage, a phosphorothioate linkage, and a neutral internucleotidic linkage (e.g., n001, n002, n003, n004, n005, n006, n007, n008, n009, n010, n013, etc.).

Oligonucleotides can comprise various numbers of natural phosphate linkages, e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more. In some embodiments, one or more (e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more) of the natural phosphate linkages in an oligonucleotide are consecutive. In some embodiments, provided oligonucleotides comprise no natural phosphate linkages. In some embodiments, provided oligonucleotides comprise one natural phosphate linkage. In some embodiments, provided oligonucleotides comprise 1 to 30 or more natural phosphate linkages.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, provided oligonucleotides comprise one or more non-negatively charged internucleotidic linkages. In some embodiments, provided oligonucleotides comprise one or more neutral internucleotidic linkages. In some embodiments, provided oligonucleotides comprise one or more phosphoryl guanidine internucleotidic linkages. In some embodiments, a neutral internucleotidic linkage or non-negatively charged internucleotidic linkage is a phosphoryl guanidine internucleotidic linkage. In some embodiments, each neutral internucleotidic linkage or non-negatively charged internucleotidic linkage is independently a phosphoryl guanidine internucleotidic linkage. In some embodiments, each neutral internucleotidic linkage and non-negatively charged internucleotidic linkage is independently n001.

In some embodiments, each internucleotidic linkage in a provided oligonucleotide is independently selected from a phosphorothioate internucleotidic linkage, a phosphoryl guanidine internucleotidic linkage, and a natural phosphate linkage. In some embodiments, each internucleotidic linkage in a provided oligonucleotide is independently selected from a phosphorothioate internucleotidic linkage, n001, and a natural phosphate linkage.

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designed oligonucleotides. In some embodiments, the present disclosure provides an oligonucleotide comprising one or more modified sugars. In some embodiments, the present disclosure provides an oligonucleotide comprising one or more modified sugars and one or more modified internucleotidic linkages, one or more of which are natural phosphate linkages.

In some embodiments, an internucleotidic linkage is aphosphoryl guanidine, phosphoryl amidine, phosphoryl isourea, phosphoryl isothiourea, phosphoryl imidate, or phosphoryl imidothioate internucleotidic linkage, e.g., those as described in US 20170362270.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. No. 9,982,257, US 20170037399, US 20180216108, WO 2017192664, WO 2017015575, WO 2017062862, WO 2018067973, WO 2017160741, WO 2017192679, WO 2017210647, WO 2018098264, WO 2018223056, WO 2018237194, or WO 2019055951, the nucleobases, sugars, internucleotidic linkages, chiral auxiliaries/reagents, and technologies for oligonucleotide synthesis (reagents, conditions, cycles, etc.) of each of which is independently incorporated herein by reference. In some embodiments, an internucleotidic linkage is described in WO 2012/030683, WO 2021/030778, WO 2019112485, US 20170362270, WO 2018156056, WO 2018056871, WO 2020/154344, WO 2020/154343, WO 2020/154342, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256, and can be utilized in accordance with the present disclosure.

In some embodiments, each internucleotidic linkage in an oligonucleotide is independently selected from a natural phosphate linkage, a phosphorothioate linkage, and a non-negatively charged internucleotidic linkage (e.g., n001). In some embodiments, each internucleotidic linkage in an oligonucleotide is independently selected from a natural phosphate linkage, a phosphorothioate linkage, and a neutral internucleotidic linkage (e.g., n001).

In some embodiments, an oligonucleotide comprises one or more nucleotides that independently comprise a phosphorus modification prone to “autorelease” under certain conditions. That is, under certain conditions, a particular phosphorus modification is designed such that it self-cleaves from the oligonucleotide to provide, e.g., a natural phosphate linkage. In some embodiments, such a phosphorus modification has a structure of —O-L-RI, wherein L is L^{B} as described herein, and R¹ is R′ as described herein. In some embodiments, a phosphorus modification has a structure of -S-L-R¹, wherein each L and R¹ is independently as described in the present disclosure. Certain examples of such phosphorus modification groups can be found in U.S. Pat. No. 9,982,257. In some embodiments, an autorelease group comprises a morpholino group. In some embodiments, an autorelease group is characterized by the ability to deliver an agent to the internucleotidic phosphorus linker, which agent facilitates further modification of the phosphorus atom such as, e.g., desulfurization. In some embodiments, the agent is water and the further modification is hydrolysis to form a natural phosphate linkage.

In some embodiments, an oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28);3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al. (Nucleic Acids Research (1999), 27(20):4071-76) reported that tert-butyl SATE pro-oligonucleotides displayed markedly increased cellular penetration compared to the parent oligonucleotide under certain conditions.

Oligonucleotides can comprise various number of natural phosphate linkages. In some embodiments, 5% or more of the internucleotidic linkages of provided oligonucleotides are natural phosphate linkages. In some embodiments, 10% or more of the internucleotidic linkages of provided oligonucleotides are natural phosphate linkages. In some embodiments, 15% or more of the internucleotidic linkages of provided oligonucleotides are natural phosphate linkages. In some embodiments, 20% or more of the internucleotidic linkages of provided oligonucleotides are natural phosphate linkages. In some embodiments, 25% or more of the internucleotidic linkages of provided oligonucleotides are natural phosphate linkages. In some embodiments, 30% or more of the internucleotidic linkages of provided oligonucleotides are natural phosphate linkages. In some embodiments, 35% or more of the internucleotidic linkages of provided oligonucleotides are natural phosphate linkages. In some embodiments, 40% or more of the internucleotidic linkages of provided oligonucleotides are natural phosphate linkages. In some embodiments, provided oligonucleotides comprises 1, 2, 3, 4, 5, 6, 7, 8, 9, 10 or more natural phosphate linkages. In some embodiments, provided oligonucleotides comprises 4, 5, 6, 7, 8, 9, 10 or more natural phosphate linkages. In some embodiments, the number of natural phosphate linkages is 2. In some embodiments, the number of natural phosphate linkages is 3. In some embodiments, the number of natural phosphate linkages is 4. In some embodiments, the number of natural phosphate linkages is 5. In some embodiments, the number of natural phosphate linkages is 6. In some embodiments, the number of natural phosphate linkages is 7. In some embodiments, the number of natural phosphate linkages is 8. In some embodiments, some or all of the natural phosphate linkages are consecutive. In some embodiments, no more than a certain number of internucleotidic linkages of the provided oligonucleotides are natural phosphate linkages, e.g., no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, or no more than 30 natural phosphate linkages. In some embodiments, provided oligonucleotides comprise no natural phosphate linkages.

In some embodiments, Sp internucleotidic linkages. among other things, at the 5′- and/or 3′-end can improve oligonucleotide stability. In some embodiments, among other things, natural phosphate linkages and/or Rp internucleotidic linkages may improve removal of oligonucleotides from a system. As appreciated by a person having ordinary skill in the art, various assays known in the art can be utilized to assess such properties in accordance with the present disclosure.

In some embodiments, each phosphorothioate internucleotidic linkage in an oligonucleotide or a portion thereof (e.g., a domain, a subdomain, etc.) is independently chirally controlled. In some embodiments, each is independently Sp or Rp. In some embodiments, a high level is Sp as described herein. In some embodiments, each phosphorothioate internucleotidic linkage in an oligonucleotide or a portion thereof is chirally controlled and is Sp. In some embodiments, one or more, e.g., about 1-5 (e.g., about 1, 2, 3, 4, or 5) is Rp.

In some embodiments, as illustrated in certain examples, an oligonucleotide or a portion thereof comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in an oligonucleotide or a portion thereof is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, two or more non-negatively charged internucleotidic linkages are consecutive. In some embodiments, no two non-negatively charged internucleotidic linkages are consecutive. In some embodiments, all non-negatively charged internucleotidic linkages in an oligonucleotide or a portion thereof are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of an oligonucleotide or a portion thereof. In some embodiments, the last two or three or four internucleotidic linkages of an oligonucleotide or a portion thereof comprise at least one internucleotidic linkage that is not a non-negatively charged internucleotidic linkage. In some embodiments, the last two or three or four internucleotidic linkages of an oligonucleotide or a portion thereof comprise at least one internucleotidic linkage that is not n001. In some embodiments, the internucleotidic linkage linking the first two nucleosides of an oligonucleotide or a portion thereof is a non-negatively charged internucleotidic linkage. In some embodiments, the internucleotidic linkage linking the last two nucleosides of an oligonucleotide or a portion thereof is a non-negatively charged internucleotidic linkage. In some embodiments, the internucleotidic linkage linking the first two nucleosides of an oligonucleotide or a portion thereof is a phosphorothioate internucleotidic linkage. In some embodiments, it is Sp. In some embodiments, the internucleotidic linkage linking the last two nucleosides of an oligonucleotide or a portion thereof is a phosphorothioate internucleotidic linkage. In some embodiments, it is Sp.

In some embodiments, one or more chiral internucleotidic linkages are chirally controlled and one or more chiral internucleotidic linkages are not chirally controlled. In some embodiments, each phosphorothioate internucleotidic linkage is independently chirally controlled, and one or more non-negatively charged internucleotidic linkage are not chirally controlled. In some embodiments, each phosphorothioate internucleotidic linkage is independently chirally controlled, and each non-negatively charged internucleotidic linkage is not chirally controlled. In some embodiments, the internucleotidic linkage between the first two nucleosides of an oligonucleotide is a non-negatively charged internucleotidic linkage. In some embodiments, the internucleotidic linkage between the last two nucleosides are each independently a non-negatively charged internucleotidic linkage. In some embodiments, both are independently non-negatively charged internucleotidic linkages. In some embodiments, an oligonucleotide comprises one or more additional internucleotidic linkages, e.g., one of which is between the nucleosides at positions −1 and -2 relative to a nucleoside opposite to a target nucleoside (e.g., a target adenosine) (the two nucleosides immediately 3′ to a nucleoside opposite to a target nucleoside (e.g., in . . . N₀N₋₁N₋₂ . . . , N₀ is a nucleoside opposite to a target nucleoside, N₋₁ and N₋₂ are at positions −1 and −2, respectively). In some embodiments, each non-negatively charged internucleotidic linkage is independently neutral internucleotidic linkage. In some embodiments, each non-negatively charged internucleotidic linkage is independently n001.

In some embodiments, non-negatively charged internucleotidic linkages such as n001 may provide improved properties and/or activities. In some embodiments, in an oligonucleotide a 5′-end internucleotidic linkage and/or a 3′-end internucleotidic linkage, each of which is independently bonded to two nucleosides comprising a nucleobase as described herein, is a non-negatively charged internucleotidic linkage as described herein. In some embodiments, the first one or more (e.g., the first 1, 2, and/or 3), and/or the last one or more (e.g., the last 1, 2, 3, 4, 5, 6 or 7) internucleotidic linkages, each of which is independently bonded to two nucleosides in a first domain, is independently a non-negatively charged internucleotidic linkage. In some embodiments, the first internucleotidic linkage of a first domain is a non-negatively charged internucleotidic linkage. In some embodiments, the last internucleotidic linkage that bonds to two nucleosides of a first domain is a non-negatively charged internucleotidic linkage. In some embodiments, the last internucleotidic linkage of a second domain is a non-negatively charged internucleotidic linkage. In some embodiments, one or more of internucleotidic linkages in the middle of a second domain, e.g., one or more of the 4^{th}, 5^{th} and 6^{th} internucleotidic linkages, each of which independently bonds to two nucleosides of a second domain, is independently a non-negatively charged internucleotidic linkage. In some embodiments, the 11^{th} internucleotidic linkage that bonds to two nucleosides of a second domain is a non-negatively charged internucleotidic linkage. In some embodiments, an internucleotidic linkage that is not bonded to a nucleoside opposite to a target nucleoside but is bonded to its 3′ immediate nucleoside is a non-negatively charged internucleotidic linkage. In some embodiments, a non-negatively charged internucleotidic linkage is a neutral internucleotidic linkage. In some embodiments, a non-negatively charged internucleotidic linkage is n001. In some embodiments, each non-negatively charged internucleotidic linkage is n001. In some embodiments, a non-negatively charged internucleotidic linkage is stereorandom. In some embodiments, a non-negatively charged internucleotidic linkage is chirally controlled and is Rp. In some embodiments, a non-negatively charged internucleotidic linkage is chirally controlled and is Sp. In some embodiments, each non-negatively charged internucleotidic linkage is independently chirally controlled. In some embodiments, one or more internucleotidic linkages of a first domain, e.g., one or more of the 4^{th}, 5^{th}, 6^{th}, 7^{th} and 8^{th} internucleotidic linkages each of which is independently bonded to two nucleosides of a first domain, is independently not a non-negatively charged internucleotidic linkage. In some embodiments, one or more internucleotidic linkages of a second domain, e.g., one or more of the 1^{st}, 2^{nd}, 3^{rd}, 7^{th}, 8^{th}, 9^{th}, 12^{th}, and 13^{th} internucleotidic linkages each of which is independently bonded to two nucleosides of a first domain, is independently not a non-negatively charged internucleotidic linkage. In some embodiments, one or both of the 2^{nd} and the 3^{rd} internucleotidic linkages of a second domain is not a non-negatively charged internucleotidic linkage. In some embodiments, an internucleotidic linkage that is not a non-negatively charged internucleotidic linkage is a phosphorothioate internucleotidic linkage. In some embodiments, it is a stereorandom phosphorothioate internucleotidic linkage. In some embodiments, it is a Rp chirally controlled phosphorothioate internucleotidic linkage. In some embodiments, it is a Sp chirally controlled phosphorothioate internucleotidic linkage.

In some embodiments, one or more or all internucleotidic linkages at positions +11, +9, +5, −2, and −5 of a nucleoside opposite to a target adenosine are independently non-negatively charged internucleotidic linkages (“+” is counting from a nucleoside opposite to a target adenosine toward the 5′-end of an oligonucleotide with the internucleotidic linkage at the +1 position being the internucleotidic linkage between a nucleoside opposite to a target adenosine and its 5′ side neighboring nucleoside (e.g., being between N₁ and N₀ of 5′-N₁N₀N₋₁-3′, wherein as described herein N₀ is a nucleoside opposite to a target adenosine), and “−” is counting from the nucleoside toward the 3′-end of an oligonucleotide with the internucleotidic linkage at the −1 position being the internucleotidic linkage between a nucleoside opposite to a target adenosine and its 3′ side neighboring nucleoside (e.g., being between N₋₁ and No of 5′-N₁N₀N₋₁-3′, wherein as described herein N₀ is the nucleoside opposite to a target adenosine)). In some embodiments, the first internucleotidic linkage of an oligonucleotide is a non-negatively charged internucleotidic linkage. In some embodiments, the last internucleotidic linkage of an oligonucleotide is a non-negatively charged internucleotidic linkage. In some embodiments, the first and last internucleotidic linkages of an oligonucleotide are each independently a non-negatively charged internucleotidic linkage. In some embodiments, one or more or all internucleotidic linkages at positions +21, +20, +18, +17, +16, +15, +14, +13, +12, +11, +10, +6, +5, +4, and −2 are independently non-negatively charged internucleotidic linkage (e.g., a phosphoryl guanidine internucleotidic linkage such as n001). In some embodiments, one or more or all internucleotidic linkages at positions +24, +23, +22, +19, +16, +15, +14, +13, +12, +11, +10, +6, +5, +4, −2, and −5 are independently non-negatively charged internucleotidic linkage (e.g., a phosphoryl guanidine internucleotidic linkage such as n001). In some embodiments, one or more or all internucleotidic linkages at positions +23, +22, +19, +16, +15, +14, +13, +12, +11, +10, +6, +5, +4, and −2 are independently non-negatively charged internucleotidic linkage (e.g., a phosphoryl guanidine internucleotidic linkage such as n001). In some embodiments, the first and last internucleotidic linkages of an oligonucleotide are independently non-negatively charged internucleotidic linkage (e.g., a phosphoryl guanidine internucleotidic linkage such as n001). In some embodiments, the first and the last internucleotidic linkages and one or more or all internucleotidic linkages at positions +23, +22, +19, +16, +15, +14, +13, +12, +11, +10, +6, +5, +4, and −2 are independently non-negatively charged internucleotidic linkage (e.g., a phosphoryl guanidine internucleotidic linkage such as n001). In some embodiments, the first and the last internucleotidic linkages are both Rp. In some embodiments, each phosphorothioate internucleotidic linkages are Sp. In some embodiments, an internucleotidic linkage at position −2 is a non-negatively charged internucleotidic linkage. In some embodiments, an internucleotidic linkage at position −5 is a non-negatively charged internucleotidic linkage. In some embodiments, an internucleotidic linkage at position +5 is a non-negatively charged internucleotidic linkage. In some embodiments, an internucleotidic linkage at position +9 is a non-negatively charged internucleotidic linkage. In some embodiments, an internucleotidic linkage at position +11 is a non-negatively charged internucleotidic linkage. In some embodiments, each of the internucleotidic linkages at positions −2, and −5 is independently a non-negatively charged internucleotidic linkage. In some embodiments, each of the internucleotidic linkages at positions +5, −2, and −5 is independently a non-negatively charged internucleotidic linkage. In some embodiments, each of the internucleotidic linkages at positions +11, +9, −2, and −5 is independently a non-negatively charged internucleotidic linkage. In some embodiments, each of the internucleotidic linkages at positions +11, +9, +5, −2, and −5 is independently a non-negatively charged internucleotidic linkage. In some embodiments, one or more or each of the 1^{st}, 14^{th}, 16^{th}, 20^{th}, 26^{th} and 29^{th} internucleotidic linkages (unless otherwise specified, from the 5′-end) is independently a non-negatively charged internucleotidic linkage. In some embodiments, an oligonucleotide comprises no non-negatively charged internucleotidic linkages to the 5′ side of a nucleoside opposite to a target adenosine except that the first internucleotidic linkage of an oligonucleotide may be optionally a non-negatively charged internucleotidic linkage. In some embodiments, an oligonucleotide comprises no internal non-negatively charged internucleotidic linkages except at position -2. In some embodiments, one or both of the first and last internucleotidic linkages of a first domain is independently a non-negatively charged internucleotidic linkage. In some embodiments, one or both of the first and last internucleotidic linkages of a second domain is independently a non-negatively charged internucleotidic linkage. In some embodiments, one or both of the first and last internucleotidic linkages of an oligonucleotide is independently a non-negatively charged internucleotidic linkage. In some embodiments, both of the first and last internucleotidic linkages of a first domain are independently non-negatively charged internucleotidic linkages. In some embodiments, both of the first and last internucleotidic linkages of a second domain are independently non-negatively charged internucleotidic linkages. In some embodiments, both of the first and last internucleotidic linkages of an oligonucleotide are independently non-negatively charged internucleotidic linkages. In some embodiments, each non-negatively charged internucleotidic linkage is independently a neutral internucleotidic linkage. In some embodiments, a non-negatively charged internucleotidic linkage is a phosphoryl guanidine internucleotidic linkage. In some embodiments, each non-negatively charged internucleotidic linkage is independently a phosphoryl guanidine internucleotidic linkage. In some embodiments, a non-negatively charged internucleotidic linkage is n001. In some embodiments, each non-negatively charged internucleotidic linkage is independently n001. In some embodiments, each non-negatively charged internucleotidic linkage is independently Rp, Sp, or non-chirally controlled. In some embodiments, one or more non-negatively charged internucleotidic linkages are independently not chirally controlled. In some embodiments, each non-negatively charged internucleotidic linkage is independently not chirally controlled. In some embodiments, one or more non-negatively charged internucleotidic linkages are independently chirally controlled. In some embodiments, each non-negatively charged internucleotidic linkage is independently chirally controlled. In some embodiments, each non-negatively charged internucleotidic linkage is Rp. In some embodiments, each non-negatively charged internucleotidic linkage is Sp. In some embodiments, an internucleotidic linkage, e.g., n001, bonded to an inosine or deoxyinosine or 2′-modified inosine (e.g., 2′-OH replaced with a non-H moiety such as —F, —OMe, —MOE, etc.) at its 3′ position is non-chirally controlled or is chirally controlled and Sp. In some embodiments, it is chirally controlled and Sp. In some embodiments, oligonucleotides and compositions thereof comprising chirally controlled Sp non-negatively charged internucleotidic linkages (e.g., phosphoryl guanidine internucleotidic linkages such as n001) bonded to 3′-positions of nucleosides comprising hypoxanthine provide various advantages over corresponding stereorandom or Rp internucleotidic linkages, e.g., the same or better properties and/or activities, improved manufacturing efficiency, and/or lowered manufacturing cost, etc. In some embodiments, processes for constructing chirally controlled Sp non-negatively charged internucleotidic linkages (e.g., phosphoryl guanidine internucleotidic linkages such as n001) bonded to 3′-positions of nucleosides comprising hypoxanthine can be performed more readily (e.g., higher reagent concentrations, smaller solution volumes, shorter reaction times, etc.) and/or with lower cost (e.g., more easily accessible materials).. In some embodiments, oligonucleotides and compositions thereof comprising chirally controlled Rp phosphorothioate internucleotidic linkages bonded to 3′-positions of nucleosides comprising hypoxanthine provide various advantages over corresponding stereorandom or Sp internucleotidic linkages, e.g., the same or better properties and/or activities, improved manufacturing efficiency, and/or lowered manufacturing cost, etc. In some embodiments, processes for constructing chirally controlled Rp phosphorothioate internucleotidic linkages bonded to 3′-positions of nucleosides comprising hypoxanthine can be performed more readily (e.g., higher reagent concentrations, smaller solution volumes, shorter reaction times, etc.) and/or with lower cost (e.g., more easily accessible materials).

In some embodiments, an oligonucleotide comprises one or more (e.g., 1-10, 1, 2, 3, 4, 5, 6, 7, 8, 9, 10 or more, etc.) natural phosphate linkages. In some embodiments, both nucleosides bonded to a natural phosphate linkage are independently a 2′-modified sugar. In some embodiments, both nucleosides bonded to a majority (e.g., at least 50%, 60%, 70%, 75%, 80%, 85%, 90%, 95% or more) of natural phosphate linkages are independently a 2′-modified sugar. In some embodiments, both nucleosides bonded to each natural phosphate linkage are independently a 2′-modified sugar. In some embodiments, a 2′-modified sugar is a bicyclic sugar or 2′-OR modified sugar wherein R is optionally substituted C₁₋₆ aliphatic. In some embodiments, each 2′-modified sugar is independently a bicyclic sugar or 2′-OR modified sugar wherein R is optionally substituted C₁₋₆ aliphatic. In some embodiments, each 2′-modified sugar is independently a 2′-OR modified sugar wherein R is optionally substituted C₁₋₆ aliphatic. In some embodiments, each 2′-modified sugar is independently a 2′-OMe modified sugar or a 2′-MOE modified sugar. In some embodiments, each 2′-modified sugar is independently a 2′-OMe modified sugar. In some embodiments, each 2′-modified sugar is independently a 2′-MOE modified sugar. In some embodiments, a natural phosphate linkage is utilized with a non-negatively charged internucleotidic linkage (e.g., a phosphoryl guanidine internucleotidic linkage such as n001). In some embodiments, an oligonucleotide comprises alternating natural phosphate linkages and non-negatively charged internucleotidic linkages (e.g., a phosphoryl guanidine internucleotidic linkage such as n001).

In some embodiments, one or more internucleotidic linkages at positions −1 and −2 are independently Rp phosphorothioate internucleotidic linkages. In some embodiments, one or more internucleotidic linkages at positions −3, −2, −1, +1, +3, +4, +5, +7, +8, +9, +10, +11, +12, +13,+16, +17 and +18 are independently Rp phosphorothioate internucleotidic linkages. In some embodiments, an internucleotidic linkage at position −3 is a Rp phosphorothioate internucleotidic linkage. In some embodiments, an internucleotidic linkage at position −2 is a Rp phosphorothioate internucleotidic linkage. In some embodiments, an internucleotidic linkage at position −1 is a Rp phosphorothioate internucleotidic linkage. In some embodiments, an internucleotidic linkage at position +1 is a Rp phosphorothioate internucleotidic linkage. In some embodiments, an internucleotidic linkage at position +3 is a Rp phosphorothioate internucleotidic linkage. In some embodiments, an internucleotidic linkage at position +4 is a Rp phosphorothioate internucleotidic linkage. In some embodiments, an internucleotidic linkage at position +5 is a Rp phosphorothioate internucleotidic linkage. In some embodiments, an internucleotidic linkage at position +7 is a Rp phosphorothioate internucleotidic linkage. In some embodiments, an internucleotidic linkage at position +8 is a Rp phosphorothioate internucleotidic linkage. In some embodiments, an internucleotidic linkage at position +9 is a Rp phosphorothioate internucleotidic linkage. In some embodiments, an internucleotidic linkage at position +10 is a Rp phosphorothioate internucleotidic linkage. In some embodiments, an internucleotidic linkage at position +11 is a Rp phosphorothioate internucleotidic linkage. In some embodiments, an internucleotidic linkage at position +12 is a Rp phosphorothioate internucleotidic linkage. In some embodiments, an internucleotidic linkage at position +13 is a Rp phosphorothioate internucleotidic linkage. In some embodiments, an internucleotidic linkage at position +16 is a Rp phosphorothioate internucleotidic linkage. In some embodiments, an internucleotidic linkage at position +17 is a Rp phosphorothioate internucleotidic linkage. In some embodiments, an internucleotidic linkage at position +18 is a Rp phosphorothioate internucleotidic linkage. In some embodiments, an oligonucleotide contains one and only one Rp phosphorothioate internucleotidic linkage. In some embodiments, it contains two and no more than two. In some embodiments, it contains three and no more than three. In some embodiments, it contains four and no more than four. In some embodiments, it contains five and no more than five.

In some embodiments, a non-negatively charged internucleotidic linkage bonded to 3′-carbon of dI is Sp. In some embodiments, a non-negatively charged internucleotidic linkage bonded to 3′-carbon of dI is Sp. In some embodiments, a phosphoryl guanidine internucleotidic linkage bonded to 3′-carbon of dI is Sp. In some embodiments, a n001 internucleotidic linkage bonded to 3′-carbon of dI is Sp. In some embodiments, each non-negatively charged internucleotidic linkage bonded to 3′-carbon of dI is independently Sp. In some embodiments, each neutral internucleotidic linkage bonded to 3′-carbon of dI is independently Sp. In some embodiments, each phosphoryl guanidine internucleotidic linkage bonded to 3′-carbon of dI is independently Sp. In some embodiments, each n001 bonded to 3′-carbon of dI is independently Sp.

In some embodiments, a controlled level of oligonucleotides in a composition are desired oligonucleotides. In some embodiments, of all oligonucleotides in a composition that share a common base sequence (e.g., a desired sequence for a purpose), or of all oligonucleotides in a composition, level of desired oligonucleotides (which may exist in various forms (e.g., salt forms) and typically differ only at non-chirally controlled internucleotidic linkages (various forms of the same stereoisomer can be considered the same for this purpose)) is about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%. In some embodiments, a level is at least about 50%. In some embodiments, a level is at least about 60%. In some embodiments, a level is at least about 70%. In some embodiments, a level is at least about 75%. In some embodiments, a level is at least about 80%. In some embodiments, a level is at least about 85%. In some embodiments, a level is at least about 90%. In some embodiments, a level is or is at least (DS)^{nc}, wherein DS is about 90%, 91%, 92%, 93%, 94%, 95%, 96%, 97%, 98%, 99% or 99.5% and nc is the number of chirally controlled internucleotidic linkages as described in the present disclosure (e.g., 1-50, 1-40, 1-30, 1-25, 1-20, 5-50, 5-40, 5-30, 5-25, 5-20, 1,2, 3,4,5, 6, 7, 8, 9, 10, 11, 12, 13, 14, 15, 16, 17, 18, 19, 20,21,22,23, 24,25 or more). In some embodiments, a level is or is at least (DS)^{nc}, wherein DS is 95%-100%.

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designing oligonucleotides. In some embodiments, the present disclosure provides an oligonucleotide comprising one or more modified sugars. In some embodiments, the present disclosure provides an oligonucleotide comprising one or more modified sugars and one or more modified internucleotidic linkages, one or more of which are natural phosphate linkages.

In some embodiments, provided oligonucleotides comprise a number of natural RNA sugars (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more, two or more or all of them are optionally consecutive). In some embodiments, such oligonucleotides comprise modified sugars, e.g., 2′ modified sugars (e.g., 2′-F, etc.) and/or 2′-OR modified sugars wherein R is not —H (e.g., 2—OMe, 2-MOE, etc.) at one or both ends, and/or various modified internucleotidic linkages (e.g., phosphorothioate internucleotidic linkages, non-negatively charged internucleotidic linkages, etc.). In some embodiments, at the 5′-end there are one or more, e.g., 1, 2, 3, 4, 5, 6, 7, 8, 9, or 10 more such 2′-OR modified sugars, wherein R is not —H. In some embodiments, at the 3′-end there are one or more, e.g., 1, 2, 3, 4, 5, 6, 7, 8, 9, or 10 more such 2′-OR modified sugars, wherein R is not —H. In some embodiments, each 2′-modified sugar is independently a 2′-OR modified sugar wherein R is not —H. In some embodiments, as described herein, 2′-OR is 2′-OMe. In some embodiments, 2′-OR is 2′-MOE. In some embodiments, each of 2′-OR is independently 2′-OMe or 2′-MOE. In some embodiments, each 2′-OR is 2′-OMe.

In some embodiments, stability of various internucleotidic linkages is assessed. In some embodiments, internucleotidic linkages are exposed to various conditions utilized for oligonucleotide manufacturing, e.g., solid phase oligonucleotide synthesis, including reagents, solvents, temperatures (in some cases, temperatures higher than room temperature), cleavage conditions, deprotection conditions, purification conditions, etc., and stability is assessed. In some embodiments, stable internucleotidic linkages (e.g., those having no more than 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% degradation when exposed to one or more conditions and/or processes, or after a complete oligonucleotide manufacturing process) are selected for utilization in various oligonucleotide compositions and applications.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In some embodiments, an additional chemical moiety is or comprises a small molecule moiety. In some embodiments, a small molecule is a ligand of a protein (e.g., receptor). In some embodiments, a small molecule binds to a polypeptide. In some embodiments, a small molecule is an inhibitor of a polypeptide. In some embodiments, an additional chemical moiety is or comprises a peptide moiety (e.g., an antibody). In some embodiments, an additional chemical moiety is or comprises a nucleic acid moiety. In some embodiments, a nucleic acid provides a new property and/or activity. In some embodiments, a nucleic acid moiety forms a duplex or other secondary structure with the original oligonucleotide chain (before conjugation) or a portion thereof. In some embodiments, a nucleic acid is or comprises an oligonucleotide targeting the same or a different target, and may perform its activity through the same or a different mechanism. In some embodiments, a nucleic acid is or comprises a RNAi agent. In some embodiments, a nucleic acid is or comprises a miRNA agent. In some embodiments, a nucleic acid is or comprises RNase H dependent. In some embodiments, a nucleic acid is or comprises a gRNA. In some embodiments, a nucleic acid is or comprises an aptamer. In some embodiments, an additional chemical moiety is or comprises a carbohydrate moiety as described herein. Many useful agents, e.g., small molecules, peptides, carbohydrates, nucleic acid agents, etc., may be conjugated with oligonucleotides herein in accordance with the present disclosure.

In some embodiments, an oligonucleotide comprises an additional chemical moiety can provide increased delivery to and/or activity in an tissue compared to a reference oligonucleotide, e.g., a reference oligonucleotide which does not have the additional chemical moiety but is otherwise identical.

In some embodiments, non-limiting examples of additional chemical moieties include carbohydrate moieties, targeting moieties, etc., which, when incorporated into oligonucleotides, can improve one or more properties. In some embodiments, an additional chemical moiety is selected from: glucose, GluNAc (N-acetyl amine glucosamine) and anisamide moieties. In some embodiments, a provided oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, a ligand moiety is or comprises a GalNAc moiety, which may be a ligand moiety for an asialoglycoprotein receptor. In some embodiments, an additional chemical moiety facilitates delivery to liver.

In some embodiments, a provided oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In some embodiments, a carbohydrate moiety is a targeting moiety. In some embodiments, a targeting moiety is a carbohydrate moiety.

In some embodiments, a provided oligonucleotide comprises an additional chemical moiety suitable for delivery, e.g., glucose, GluNAc (N-acetyl amine glucosamine), anisamide, or a structure selected from:

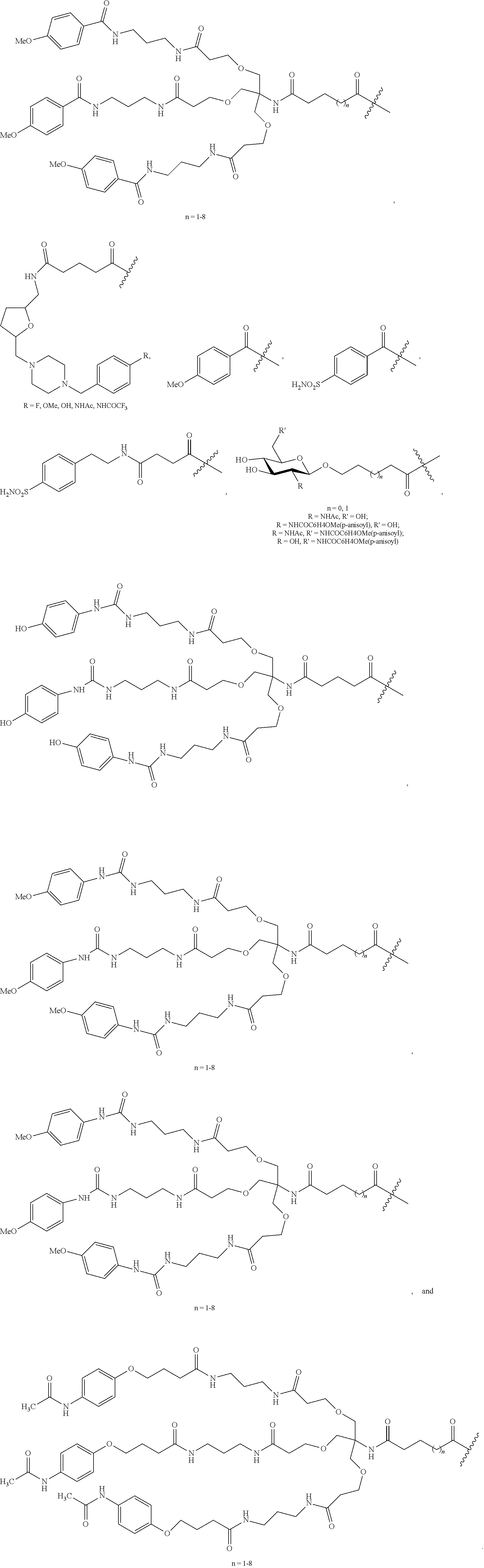

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, additional chemical moieties are any of ones described in the Examples, including examples of various additional chemical moieties incorporated into various oligonucleotides.

In some embodiments, an additional chemical moiety conjugated to an oligonucleotide is capable of targeting the oligonucleotide to a cell in the central nervous system.

In some embodiments, an additional chemical moiety comprises or is a cell receptor ligand. In some embodiments, an additional chemical moiety comprises or is a protein binder, e.g.. one binds to a cell surface protein. Such moieties among other things can be useful for targeted delivery of oligonucleotides to cells expressing the corresponding receptors or proteins. In some embodiments, an additional chemical moiety of a provided oligonucleotide comprises anisamide or a derivative or an analog thereof and is capable of targeting the oligonucleotide to a cell expressing a particular receptor, such as the sigma 1 receptor.

In some embodiments, a provided oligonucleotide is formulated for administration to a body cell and/or tissue expressing its target. In some embodiments, an additional chemical moiety conjugated to an oligonucleotide is capable of targeting the oligonucleotide to a cell.

In some embodiments, an additional chemical moiety is selected from optionally substituted phenyl,

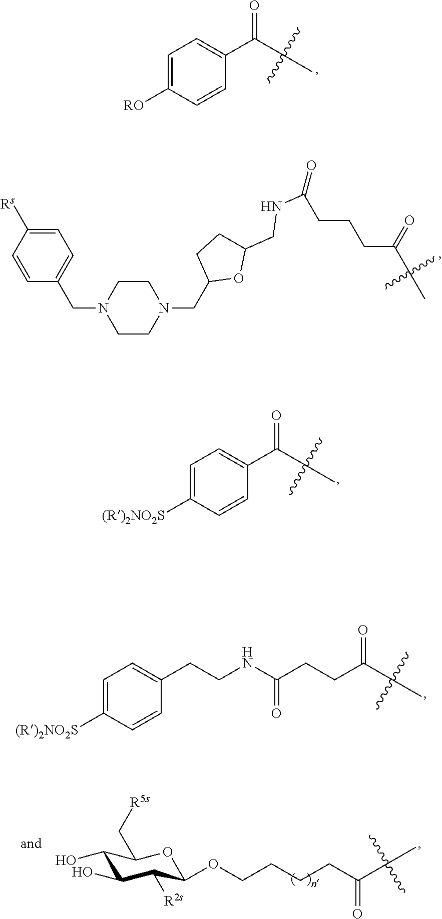

wherein n′ is 1, 2, 3, 4, 5, 6, 7, 8, 9, or 10, and each other variable is as described in the present disclosure. In some embodiments, R^{S} is F. In some embodiments, R^{s} is OMe. In some embodiments, R^{s} is OH. In some embodiments, R^{s} is NHAc. In some embodiments, R^{s} is NHCOCF₃. In some embodiments, R′ is H. In some embodiments, R is H. In some embodiments, R^{2s} is NHAc, and R^{5s} is OH. In some embodiments, R^{2s} is p-anisoyl, and R^{5s} is OH. In some embodiments, R^{2s} is NHAc and R^{5s} is p-anisoyl. In some embodiments, R^{2s} is OH, and R^{5s} is p-anisoyl. In some embodiments, an additional chemical moiety is selected from

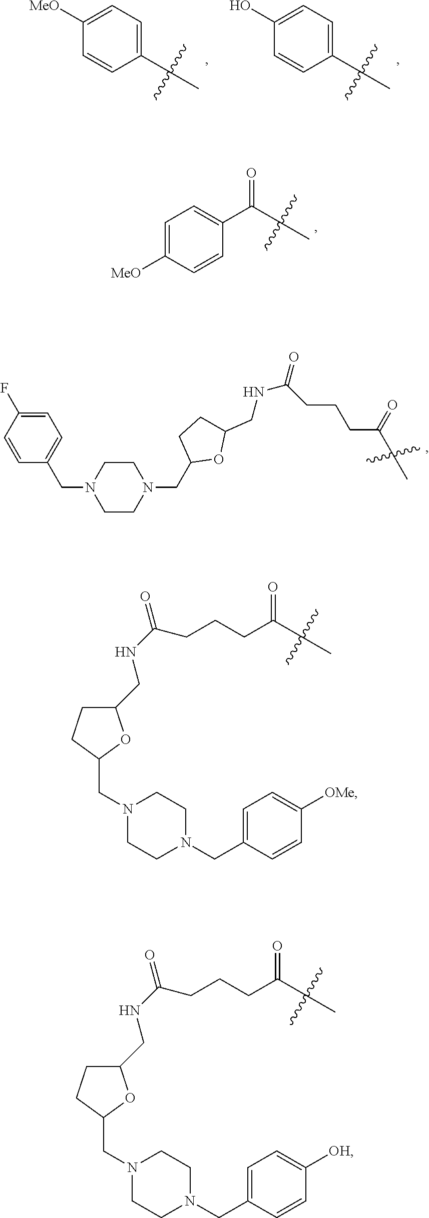 [SMILES: *c1ccc(OC)cc1 ; *c1ccc(O)cc1 ; *C(=O)c1ccc(OC)cc1 ; *C(=O)CCCC(=O)NCC1CCC(CN2CCN(Cc3ccc(F)cc3)CC2)O1 ; *C(=O)CCCC(=O)NCC1CCC(CN2CCN(Cc3ccc(OC)cc3)CC2)O1 ; *C(=O)CCCC(=O)NCC1CCC(CN2CCN(Cc3ccc(O)cc3)CC2)O1]

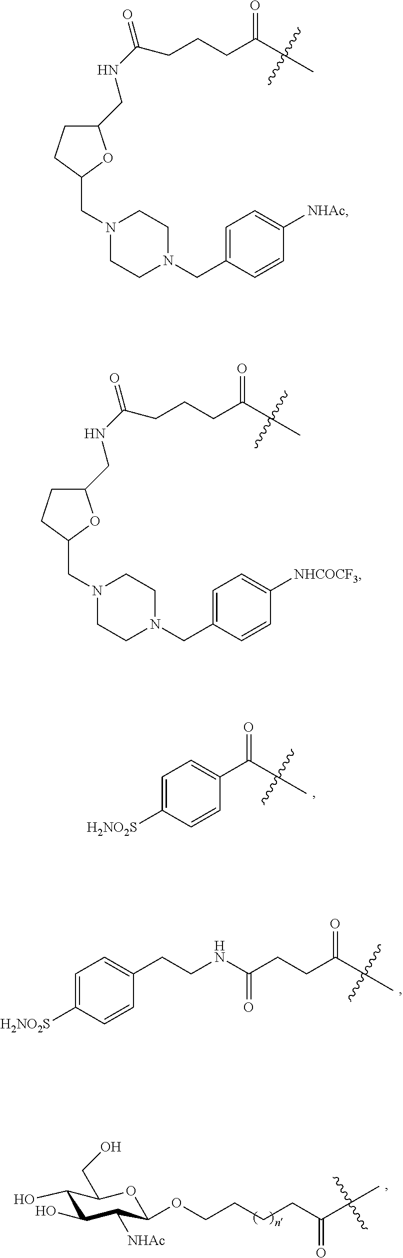

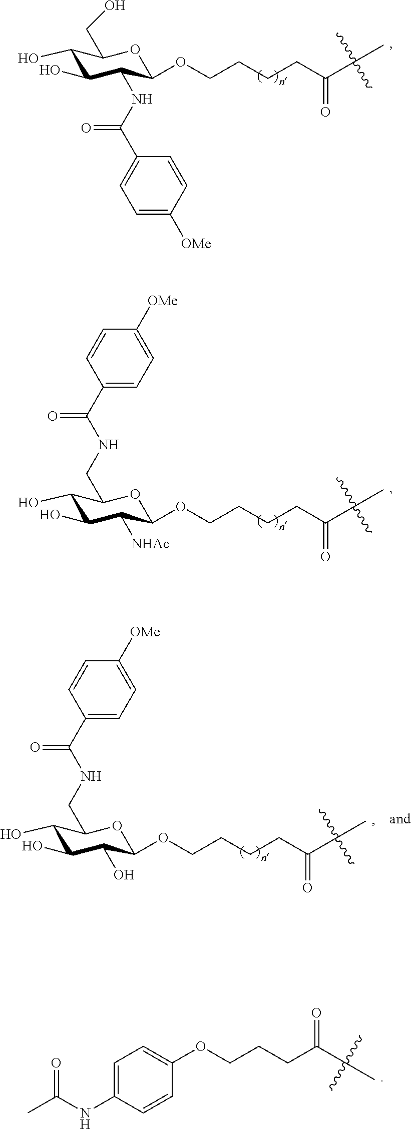

In some embodiments, n′ is 1. In some embodiments, n′ is 0. In some embodiments, n″ is 1. In some embodiments, n″ is 2.

In some embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-mal).org/experiment/show/2048

Various other ASGPR ligands are known in the art and can be utilized in accordance with the present disclosure. In some embodiments, an ASGPR ligand is a carbohydrate. In some embodiments, an ASGPR ligand is GalNac or a derivative or an analog thereof. In some embodiments, an ASGPR ligand is one described in Sanhueza et al. J. Am. Chem. Soc., 2017, 139 (9), pp 3528-3536. In some embodiments, an ASGPR ligand is one described in Mamidyala et al. J. Am. Chem. Soc., 2012, 134, pp 1978-1981. In some embodiments, an ASGPR ligand is one described in US 20160207953. In some embodiments, an ASGPR ligand is a substituted-6,8-dioxabicyclo[3.2.1]octane-2,3-diol derivative disclosed in, e.g., US 20160207953. In some embodiments, an ASGPR ligand is one described in, e.g., US 20150329555. In some embodiments, an ASGPR ligand is a substituted-6,8-dioxabicyclo[3.2.1]octane-2,3-diol derivative disclosed e.g., in US 20150329555. In some embodiments, an ASGPR ligand is one described in U.S. Pat. No. 8,877,917, US 20160376585, U.S. Ser. No. 10/086,081, or U.S. Pat. No. 8,106,022. In some embodiments, various GalNAc derivatives and uses thereof are described in WO 2022/076922 and can be utilized in accordance with the present disclosure. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In some embodiments, a provided oligonucleotide is conjugated to an ASGPR ligand. In some embodiments, a provided oligonucleotide comprises an ASGPR ligand. In some embodiments, an additional chemical moiety comprises an ASGPR ligand is

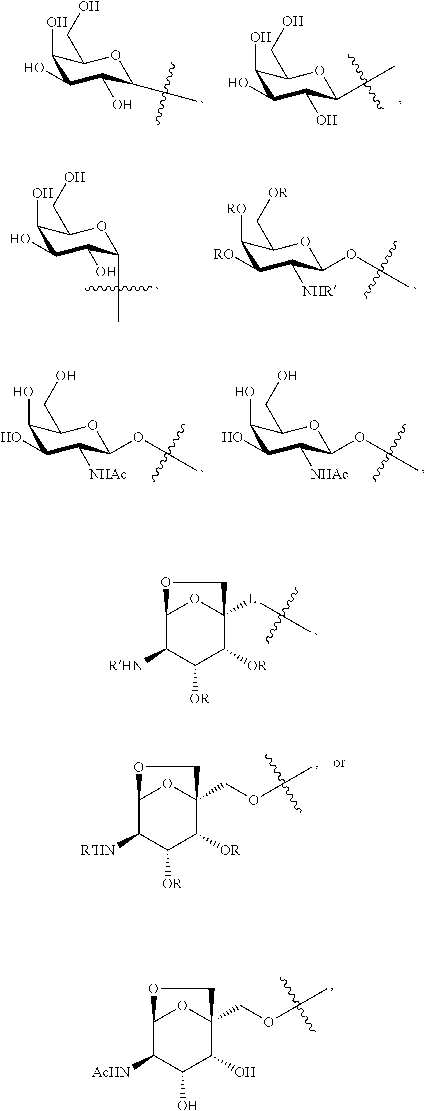

wherein each variable is independently as described in the present disclosure. In some embodiments, R is —H. In some embodiments, R′ is —C(O)R.

In some embodiments, an additional chemical moiety is or comprises

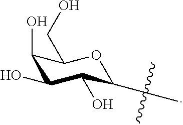

In some embodiments, an additional chemical moiety is or comprises

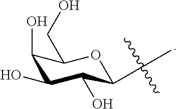

In some embodiments, an additional chemical moiety is or comprises

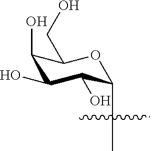

In some embodiments, an additional chemical moiety is or comprises

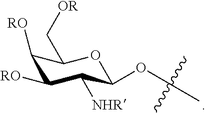

In some embodiments, an additional chemical moiety is or comprises optionally substituted

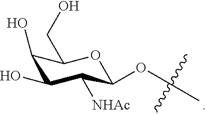

In some embodiments, an additional chemical moiety is or comprises

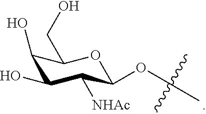

In some embodiments, an additional chemical moiety is or comprises

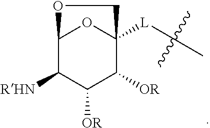

In some embodiments, an additional chemical moiety is or comprises

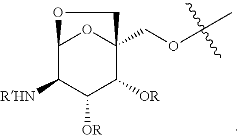

In some embodiments, an additional chemical moiety is or comprises

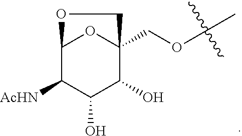 [SMILES: *OC[C@@]12CO[C@@H](O1)[C@H](NC(C)=O)[C@@H](O)[C@H]2O]

In some embodiments, an additional chemical moiety comprises one or more moieties that can bind to, e.g., oligonucleotide target cells. For example, in some embodiments, an additional chemistry moiety comprises one or more protein ligand moieties, e.g., in some embodiments, an additional chemical moiety comprises multiple moieties, each of which independently is an ASGPR ligand. In some embodiments, as in Mod 001 and Mod083, an additional chemical moiety comprises three such ligands.

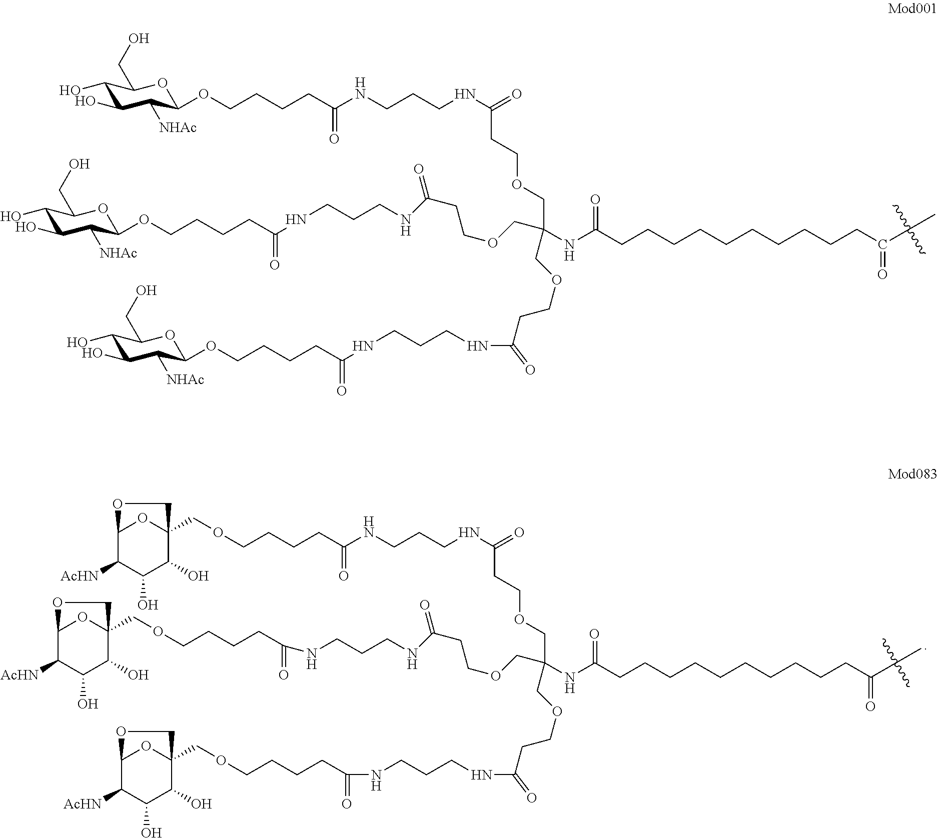

In some embodiments, an oligonucleotide comprises

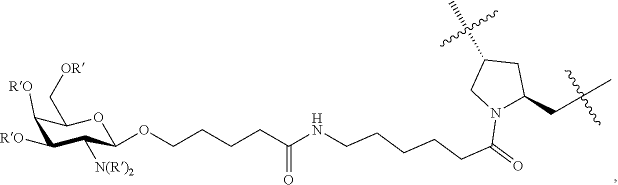

wherein each variable is independently as described herein. In some embodiments, each —OR′ is —OAc, and —N(R′)₂ is —NHAc. In some embodiments, an oligonucleotide comprises

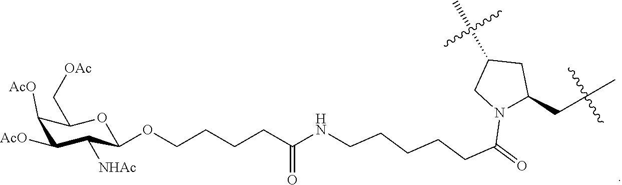

In some embodiments, each R′ is —H. In some embodiments, each —OR is —OH, and each —N(R′)₂ is —NHC(O)R. In some embodiments, each —OR′ is —OH, and each —N(R′)₂ is —NHAc. In some embodiments, an oligonucleotide comprises

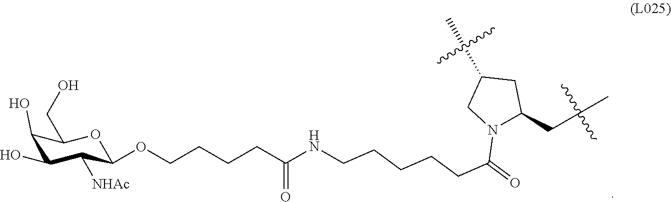

In some embodiments, the —CH₂— connection site is utilized as a C5 connection site in a sugar. In some embodiments, the connection site on the ring is utilized as a C3 connection site in a sugar. Such moieties may be introduced utilizing, e.g., phosphoramidites such as

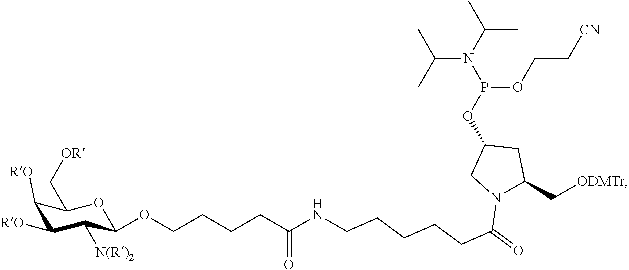

e.g.,

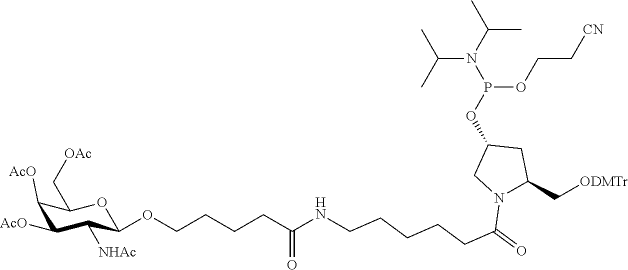

(those skilled in the art appreciate that one or more other groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3)

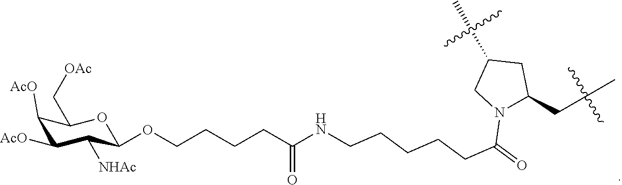

In some embodiments, an oligonucleotide comprises 2, 3 or more (e.g., 3 and no more than 3)

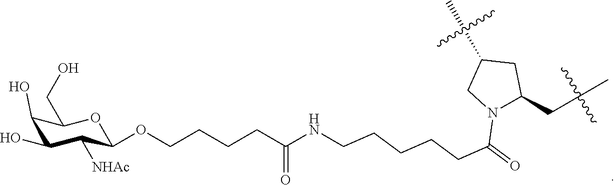

In some embodiments, copies of such moieties are linked by internucleotidic linkages, e.g., natural phosphate linkages, as described herein. In some embodiments, when at a 5′-end, a —CH₂- connection site is bonded to —OH. In some embodiments, an oligonucleotide comprises

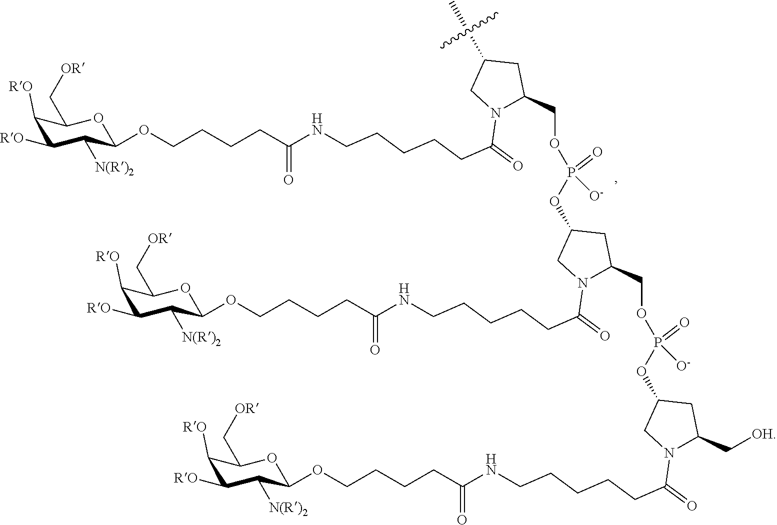

In some embodiments, an oligonucleotide comprises

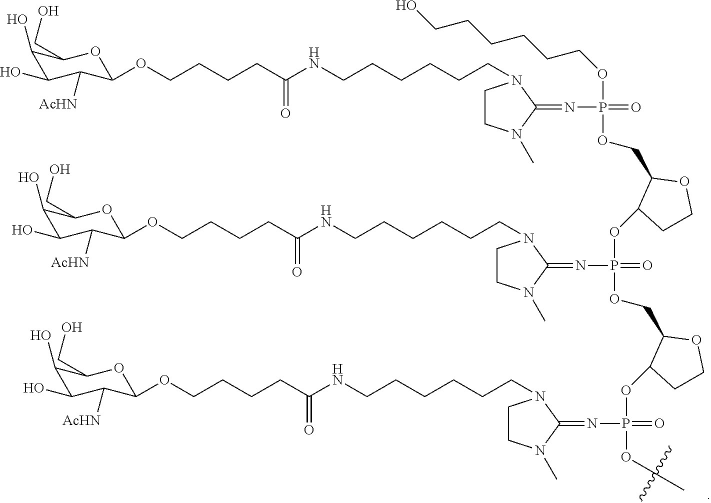 [SMILES: *OP(=O)(N=C1N(C)CCN1CCCCCCNC(=O)CCCCOC1OC(CO)C(O)C(O)C1NC(C)=O)OC1CCO[C@@H]1COP(=O)(N=C1N(C)CCN1CCCCCCNC(=O)CCCCOC1OC(CO)C(O)C(O)C1NC(C)=O)OC1CCO[C@@H]1COP(=O)(N=C1N(C)CCN1CCCCCCNC(=O)CCCCOC1OC(CO)C(O)C(O)C1NC(C)=O)OCCCCCCO]

In some embodiments, each —OR′ is OAc, and N(R′)₂ is NHAc. In some embodiments, an oligonucleotide comprises

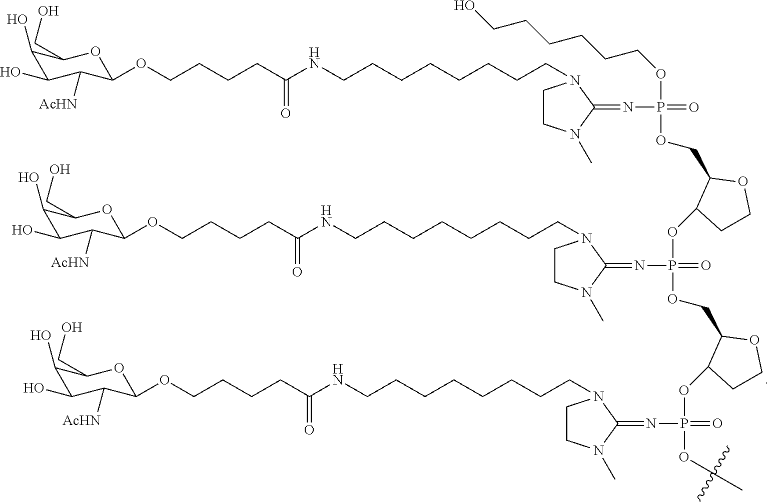 [SMILES: *OP(=O)(N=C1N(C)CCN1CCCCCCCCNC(=O)CCCCOC1OC(CO)C(O)C(O)C1NC(C)=O)OC1CCO[C@@H]1COP(=O)(N=C1N(C)CCN1CCCCCCCCNC(=O)CCCCOC1OC(CO)C(O)C(O)C1NC(C)=O)OC1CCO[C@@H]1COP(=O)(N=C1N(C)CCN1CCCCCCCCNC(=O)CCCCOC1OC(CO)C(O)C(O)C1NC(C)=O)OCCCCCCO]

Among other things,

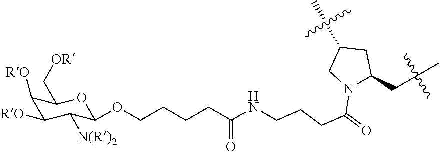

may be utilized to introduce

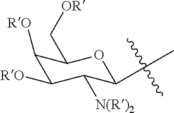

with comparable and/or better activities and/or properties. In some embodiments, it provides improved preparation efficiency and/or lower cost for the same number of

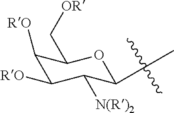

(e.g., when compared to Mod001).

In some embodiments, an additional chemical moiety is a Mod group described herein, e.g., in Table 1.

In some embodiments, an additional chemical moiety is Mod001. In some embodiments, an additional chemical moiety is Mod083. In some embodiments, an additional chemical moiety, e.g., a Mod group, is directly conjugated (e.g., without a linker) to the remainder of the oligonucleotide. In some embodiments, an additional chemical moiety is conjugated via a linker to the remainder of the oligonucleotide. In some embodiments, additional chemical moieties, e.g., Mod groups, may be directly connected, and/or via a linker, to nucleobases, sugars and/or internucleotidic linkages of oligonucleotides. In some embodiments, Mod groups are connected, either directly or via a linker, to sugars. In some embodiments, Mod groups are connected, either directly or via a linker, to 5′-end sugars. In some embodiments, Mod groups are connected, either directly or via a linker, to 5′-end sugars via 5′ carbon. For examples, see various oligonucleotides in Table 1. In some embodiments, Mod groups are connected, either directly or via a linker, to 3′-end sugars. In some embodiments, Mod groups are connected, either directly or via a linker, to 3′-end sugars via 3′ carbon. In some embodiments, Mod groups are connected, either directly or via a linker, to nucleobases. In some embodiments, Mod groups are connected, either directly or via a linker, to internucleotidic linkages. In some embodiments, provided oligonucleotides comprise Mod001 connected to 5′-end of oligonucleotide chains through L001.

As appreciated by those skilled in the art, an additional chemical moiety may be connected to an oligonucleotide chain at various locations, e.g., 5′-end, 3′-end, or a location in the middle (e.g., on a sugar, a base, an internucleotidic linkage, etc.). In some embodiments, it is connected at a 5′-end. In some embodiments, it is connected at a 3′-end. In some embodiments, it is connected at a nucleotide in the middle.

Certain additional chemical moieties (e.g., lipid moieties, targeting moieties, carbohydrate moieties), including but not limited to Mod012, Mod039, Mod062, Mod085, Mod086, and Mod094, and various linkers for connecting additional chemical moieties to oligonucleotide chains, including but not limited to L001, L003, L004, L008, L009, and L010, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the additional chemical moieties and linkers of each of which are independently incorporated herein by reference, and can be utilized in accordance with the present disclosure. In some embodiments, an additional chemical moiety is digoxigenin or biotin or a derivative thereof.

In some embodiments, an oligonucleotide comprises a linker, e.g., L001 L004, L008, and/or an additional chemical moiety, e.g., Mod012, Mod039, Mod062, Mod085, Mod086, or Mod094. In some embodiments, a linker, e.g., L001, L003, L004, L008, L009, L110, etc. is linked to a Mod, e.g., Mod012, Mod039, Mod062, Mod085, Mod086, Mod094, etc. L001: —NH—(CH₂)₆- linker (also known as a C6 linker, C6 amine linker or C6 amino linker), connected to Mod, if any, through —NH—, and the 5′-end or 3′-end of the oligonucleotide chain through either a phosphate linkage (—O—P(O)(OH)-O—, which may exist as a salt form, and may be indicated as O or PO) or a phosphorothioate linkage (-O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂- connecting site. If no Mod is present, L001 is connected to —H through —NH—;
L003:

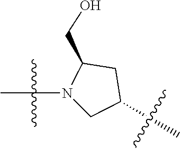

linker. In some embodiments, it is connected to Mod, if any (if no Mod, —H), through its amino group, and the 5′-end or 3′-end of an oligonucleotide chain e.g., via a linkage (e.g., a phosphate linkage (O or PO) or a phosphorothioate linkage (can be either not chirally controlled or chirally controlled (Sp or Rp)));
L004: linker having the structure of —NH(CH₂)4CH(CH₂OH)CH₂—, wherein —NH— is connected to Mod (through —C(O)-) or —H, and the —CH₂- connecting site is connected to an oligonucleotide chain (e.g., at the 3′-end) through a linkage, e.g., phosphodiester (-O—P(O)(OH)-O—, which may exist as a salt form, and may be indicated as O or PO), phosphorothioate (—O—P(O)(SH)-O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)-O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂- site) through a phosphodiester linkage to the 3′ position of the 3′-terminal sugar (which is 2′-OMe modified and connected to the nucleobase A), and the L004 linker is connected via —NH- to —H. Similarly, in one or more oligonucleotides, the L004 linker is connected (via the —CH₂- site) through the phosphodiester linkage to the 3′ position of the 3′-terminal sugar, and the L004 is connected via —NH- to, e.g., Mod012, Mod085, Mod086, etc.;
L008: linker having the structure of —C(O)-(CH₂)₉-, wherein —C(O)— is connected to Mod (through —NH-) or —OH (if no Mod indicated), and the —CH₂- connecting site is connected to an oligonucleotide chain (e.g., at the 5′-end) through a linkage, e.g., phosphodiester (-O—P(O)(OH)-O—, which may exist as a salt form, and may be indicated as O or PO), phosphorothioate (—O—P(O)(SH)-O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)-O—, which may exist as a salt form, and may be indicated as PS2 or: or D) linkage. For example, in an example oligonucleotide which has the sequence of 5′-L008 mN*mN*mN*mN*N*N*N*N*N*N*N*N*N*N*mN*mN*mN*mN-3′, and which has a Stereochemistry/Linkage of OXXXXXXXXX XXXXXXXX, wherein N is a base, wherein O is a natural phosphate internucleotidic linkage, and wherein X is a stereorandom phosphorothioate, L008 is connected to —OH through —C(O)-, and the 5′-end of an oligonucleotide chain through a phosphate linkage (indicated as “O” in “Stereochemistry/Linkage”); in another example oligonucleotide, which has the sequence of 5′-Mod062LO08 mN * mN * mN * mN * N * N * N * N * N * N * N * N * N * N * mN * mN * mN * mN-3′, and which has a Stereochemistry/Linkage of OXXXXXXXXX XXXXXXXX, wherein N is a base, L008 is connected to Mod062 through —C(O)-, and the 5′-end of an oligonucleotide chain through a phosphate linkage (indicated as “O” in “Stereochemistry/Linkage”);
L009: —CH₂CH₂CH₂—. In some embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp)));
L010:

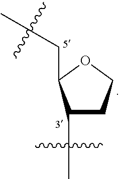

L010 connects to other moieties, e.g., L023, L010, oligonucleotide chains, etc., through various linkages (e.g., n001; if not indicated, typically phosphates). When no other moieties are present, L010 is bonded to —OH. In some embodiments, L010 is utilized with n001R to form L010n001R, which has the structure of

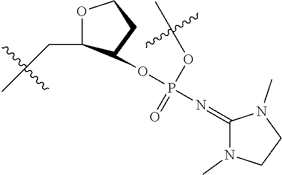

and wherein the configuration of linkage phosphorus is Rp. In some embodiments, multiple 1010n001R may be utilized. In some embodiments, an oligonucleotide comprises L023L010n001RL010n001RL010n001R, which has the following structure (which is bonded to the 5′-carbon at the 5′-end of the oligonucleotide chain, and each linkage phosphorus is independently Rp):

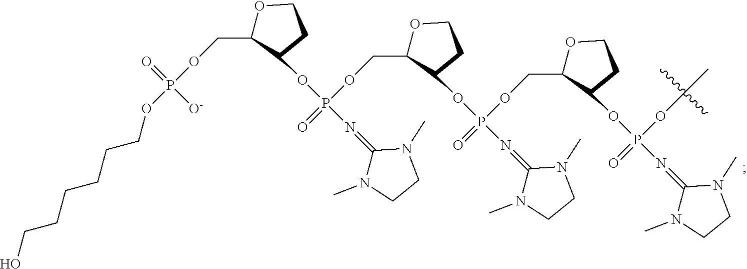

Mod012 (in some embodiments, —C(O)- connects to —NH- of a linker such as LOO1, L004, L008, etc.):

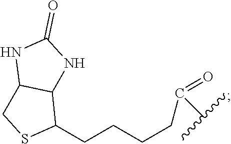 [SMILES: *C(=O)CCCCC1SCC2NC(=O)NC21]

Mod039 (in some embodiments, —C(O)- connects to —NH- of a linker such as L001, L003, L004, L008, L009, L110, etc.):

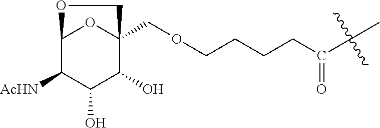 [SMILES: *C(=O)CCCCOC[C@@]12CO[C@@H](O1)[C@H](NC(C)=O)[C@@H](O)[C@H]2O]

Mod062 (in some embodiments, —C(O)- connects to —NH- of a linker such as L001, L003, L004, L008, L009,

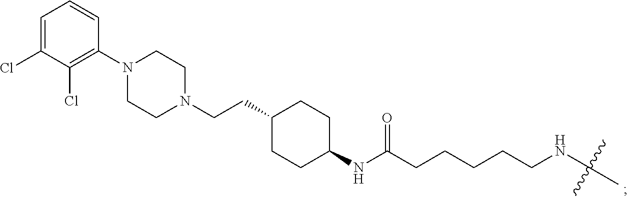 [SMILES: *NCCCCCC(=O)N[C@H]1CC[C@H](CCN2CCN(c3cccc(Cl)c3Cl)CC2)CC1]

Mod085 (in some embodiments, —C(O)- connects to —NH- of a linker such as L001, L003, L004, L008, L009, L110, etc.):

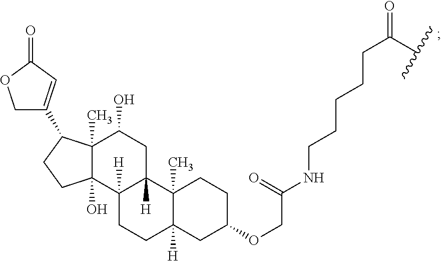

Mod086 (in some embodiments, —C(O)- connects to —NH- of a linker such as L001, L003, L004, L008, L009, L110, etc.):

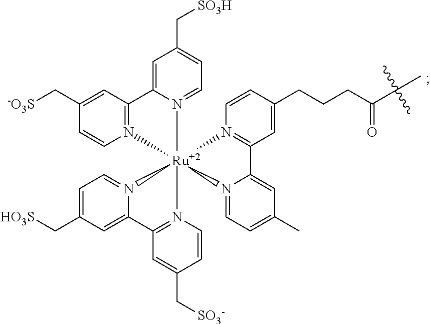

Mod094 (in some embodiments, connects to an internucleotidic linkage, or to the 5′-end or 3′-end of an oligonucleotide via a linkage, e.g., a phosphate linkage, a phosphorothioate linkage (which is optionally chirally controlled), etc.. For example, in an example oligonucleotide which has the sequence of 5′-mN * mN * mN * mN * N * N * N * N * N * N * N * N * N * N * mN * mN * mN * mNMod094-3′, and which has a Stereochemistry/Linkage of XXXXX XXXXX XXXXX XXO, wherein N is a base, Mod094 is connected to the 3′-end of the oligonucleotide chain (3′-carbon of the 3′-end sugar) through a phosphate group (which is not shown below and which may exist as a salt form; and which is indicated as “O” in “Stereochemistry/Linkage” ( . . . XXXXO))):

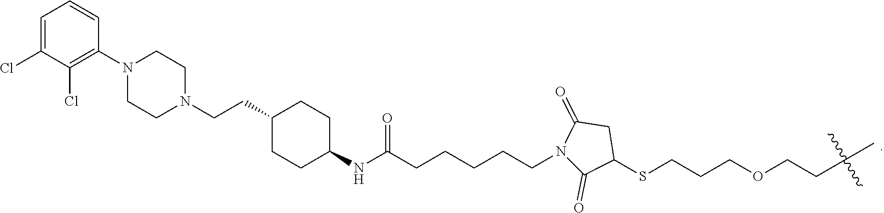 [SMILES: *CCOCCCSC1CC(=O)N(CCCCCC(=O)N[C@H]2CC[C@H](CCN3CCN(c4cccc(Cl)c4Cl)CC3)CC2)C1=O]

In some embodiments, an additional chemical moiety (e.g., a linker, lipid, solubilizing group, conjugate group, targeting group, and/or targeting ligand) is one described in WO 2012/030683 or WO 2021/030778. In some embodiments, a provided oligonucleotide comprise a chemical structure (e.g. , a linker, lipid, solubilizing group, and/or targeting ligand) described in WO 2012/030683, WO 2021/030778, WO 2019112485, US 20170362270, WO 2018156056, or WO 2018056871, WO 2021/030778, WO 2020/154344, WO 2020/154343, WO 2020/154342, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256.

In some embodiments, a provide oligonucleotide comprises an additional chemical moiety (e.g., a targeting group, a conjugate group, etc.) and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313: 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In some embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the linker moieties of each which are independently incorporated herein by reference. In some embodiments, a linker is, as non-limiting examples, L001, L004, L009 or L010. In some embodiments, an oligonucleotide comprises a linker, but not an additional chemical moiety other than the linker. In some embodiments, an oligonucleotide comprises a linker, but not an additional chemical moiety other than the linker, wherein the linker is L001, L004, L009, or L010. In some embodiments, a linker is or comprises a moiety having the structure of an internucleotidic linkage as described herein. In some embodiments, such a moiety in a linker does not connect two nucleosides. In some embodiments, a linker has the structure of L. In some embodiments, a linker is bivalent. In some embodiments, a linker is polyvalent. In some embodiments, a linker can connect two or more additional chemical moieties to an oligonucleotide chain as described herein. For example, some embodiments, one or two or three or more additional chemical moieties, e.g., GalNAc moieties, are connected to an oligonucleotide chain (e.g., at 5′-end) through a multivalent linker moiety.

In some embodiments, an additional chemical moiety is cleaved from the remainder of an oligonucleotide, e.g., an oligonucleotide chain, e.g., after administration to a system, cell, tissue, organ, subject, etc. In some embodiments, additional chemical moieties promote, increase, and/or accelerate delivery to certain cells, and after delivery of oligonucleotides into such cells, additional chemical moieties are cleaved from oligonucleotides. In some embodiments, linker moieties comprise one or more cleavable moieties that can be cleaved at desirable locations (e.g., within certain type of cells, subcellular compartments such as lysosomes, etc.) and/or timing. In some embodiments, a cleavable moiety is selectively cleaved by a polypeptide, e.g., an enzyme such as a nuclease. Many useful cleavable moieties and cleavable linkers are reported and can be utilized in accordance with the present disclosure. In some embodiments, a cleavable moiety is or comprises one or more functional groups selected from amide, ester, ether, phosphodiester, disulfide, carbamate, etc. In some embodiments, a linker is as described in WO 2012/030683, WO 2021/030778, WO 2020/154344, WO 2020/154343, WO 2020/154342, WO 2020/165077, WO 2020/201406, WO 2020/216637, or WO 2020/252376.

Among other things, provided technologies can provide high levels of activities and/or desired properties, in some embodiments, without utilizing particular structural elements (e.g., modifications, linkage configurations and/or patterns, etc.) reported to be desired and/or necessary (e.g., those reported in WO 2019/219581), though certain such structural elements may be incorporated into oligonucleotides in combination with various other structural elements in accordance with the present disclosure. For example, in some embodiments, oligonucleotides of the present disclosure have fewer nucleosides 3′ to a nucleoside opposite to a target nucleoside (e.g., a target adenosine), contain one or more phosphorothioate internucleotidic linkages at one or more positions where a phosphorothioate internucleotidic linkage was reportedly not favored or not allowed, contain one or more Sp phosphorothioate internucleotidic linkages at one or more positions where a Sp phosphorothioate internucleotidic linkage was reportedly not favored or not allowed, contain one or more Rp phosphorothioate internucleotidic linkages at one or more positions where a Rp phosphorothioate internucleotidic linkage was reportedly not favored or not allowed, and/or contain different modifications (e.g., internucleotidic linkage modifications, sugar modifications, etc.) and/or stereochemistry at one or more locations compared to those reportedly favorable or required for certain oligonucleotide properties and/or activities (e.g., presence of 2′-MOE, absence of phosphorothioate linkages at certain positions, absence of Sp phosphorothioate linkages at certain positions, and/or absence of Rp phosphorothioate linkages at certain positions were reportedly favorable or required for certain oligonucleotide properties and/or activities; in some embodiments, provided technologies can provide desired properties and/or high activities without utilizing 2′-MOE, without avoiding phosphorothioate linkages at one or more such certain positions, without avoiding Sp phosphorothioate linkages at one or more such certain positions, and/or without avoiding Rp phosphorothioate linkages at one or more such certain positions). Additionally or alternatively, provided oligonucleotides incorporates structural elements that were not previously recognized such as utilization of certain modifications (e.g., base modifications, sugar modifications (e.g., 2′-F), linkage modifications (e.g., non-negatively charged internucleotidic linkages), additional moieties, etc.) and levels, patterns, and combinations thereof.

For example, in some embodiments, as described herein, provided oligonucleotides contain no more than 5, 6, 7, 8, 9, 10, 11 or 12 nucleosides 3′ to a nucleoside opposite to a target nucleoside (e.g., a target adenosine).

Alternatively or additionally, as described herein (e.g., illustrated in certain Examples), for structural elements 3′ to a nucleoside opposite to a target nucleoside (e.g., a target adenosine), in some embodiments, about 50%-100% (e.g., about or at least about 50%, 55%, 60%, 65%, 70%, 75%, 80%, 85%, 90%, 95%) of internucleotidic linkages 3′ to a nucleoside opposite to a target nucleoside (e.g., a target adenosine) are each independently a modified internucleotidic linkage, which is optionally chirally controlled. In some embodiments, no more than 1, 2, or 3 internucleotidic linkages 3′ to a nucleoside opposite to a target nucleoside are natural phosphate linkages. In some embodiments, no such internucleotidic linkage is natural phosphate linkages. In some embodiments, no more than 1 such internucleotidic linkage is natural phosphate linkages. In some embodiments, no more than 2 such internucleotidic linkages are natural phosphate linkages. In some embodiments, no more than 3 such internucleotidic linkages are natural phosphate linkages. In some embodiments, each modified internucleotidic linkage is independently a phosphorothioate or a non-negatively charged internucleotidic linkage (e.g., n001). In some embodiments, each phosphorothioate internucleotidic linkage is chirally controlled. In some embodiments, no more than 1, 2, or 3 internucleotidic linkages 3′ to a nucleoside opposite to a target nucleoside are Rp phosphorothioate internucleotidic linkage. In some embodiments, an internucleotidic linkage bonded to a nucleoside opposite to a target nucleoside at the 3′-position of its sugar (considered a −1 position) is a Rp phosphorothioate internucleotidic linkage. In some embodiments, it is the only Rp phosphorothioate internucleotidic linkage 3′ to a nucleoside opposite to a target nucleoside. In some embodiments, an internucleotidic linkage at position −3 relative to a nucleoside opposite to a target nucleoside (e.g., for . . . N₀N₋₁N₋₂N₋₃ . . . , the internucleotidic linkage linking N₋₂ and N₋₃ wherein No is a nucleoside opposite to a target nucleoside) is not a Rp phosphorothioate internucleotidic linkage. In some embodiments, an internucleotidic linkage at position −6 relative to a nucleoside opposite to a target nucleoside is not a Rp phosphorothioate internucleotidic linkage. In some embodiments, an internucleotidic linkage at position −4 and/or −5 relative to a nucleoside opposite to a target nucleoside is independently a modified internucleotidic linkage, e.g., a phosphorothioate internucleotidic linkage, or is independently a Rp phosphorothioate internucleotidic linkage. In some embodiments, one or more or all internucleotidic linkages at positions −1, −3, −4, −5, and −6 are each independently a Sp internucleotidic linkage. In some embodiments, one or more or all internucleotidic linkages at positions −1, −3, −4, −5, and −6 are each independently a Sp phosphorothioate internucleotidic linkage. In some embodiments, internucleotidic linkage(s) at position(s) −4 and/or −5 are each independently a Rp internucleotidic linkage. In some embodiments, internucleotidic linkage(s) at position(s) −4 and/or −5 are each independently a Rp phosphorothioate internucleotidic linkage. In many embodiments, no more than 1, 2, 3, 4, or 5 internucleotidic linkages are Rp phosphorothioate internucleotidic linkage.

Alternatively or additionally, as described herein (e.g., illustrated in certain Examples), in some embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently a modified internucleotidic linkage, which is optionally chirally controlled. In some embodiments, no or no more than 1, 2, or 3 internucleotidic linkages 5′ to a nucleoside opposite to a target nucleoside (e.g., a target adenosine) are not modified internucleotidic linkages. In some embodiments, no or no more than 1, 2, or 3 internucleotidic linkages 5′ to a nucleoside opposite to a target nucleoside (e.g., a target adenosine) are not phosphorothioate internucleotidic linkages. In some embodiments, no or no more than 1, 2, or 3 internucleotidic linkages 5′ to a nucleoside opposite to a target nucleoside (e.g., a target adenosine) are not Sp phosphorothioate internucleotidic linkages. In some embodiments, no more than 1, 2, or 3 internucleotidic linkages 5′ to a nucleoside opposite to a target nucleoside (e.g., a target adenosine) are natural phosphate linkages. In some embodiments, no such internucleotidic linkage is natural phosphate linkages. In some embodiments, no more than 1 such internucleotidic linkage is natural phosphate linkages. In some embodiments, no more than 2 such internucleotidic linkages are natural phosphate linkages. In some embodiments, no more than 3 such internucleotidic linkages are natural phosphate linkages. In some embodiments, each modified internucleotidic linkage is independently a phosphorothioate or a non-negatively charged internucleotidic linkage (e.g., n001). In some embodiments, there are no 2, 3, or 4 consecutive internucleotidic linkages 5′ to a nucleoside opposite to a target nucleoside, each of which is not a phosphorothioate internucleotidic linkage. In some embodiments, there are no 2, 3, or 4 consecutive internucleotidic linkages 5′ to a nucleoside opposite to a target nucleoside, each of which is chirally controlled and is not a Sp phosphorothioate internucleotidic linkage. In some embodiments, no or no more than 1, 2, 3, 4, or 5 internucleotidic linkages 5′ to a nucleoside opposite to a target nucleoside (e.g., a target adenosine) are Rp phosphorothioate internucleotidic linkage. In some embodiments, an internucleotidic linkage bonded to a nucleoside opposite to a target nucleoside at the 5′-position of its sugar (considered a +1 position) is a Rp phosphorothioate internucleotidic linkage. In some embodiments, it is the only Rp phosphorothioate internucleotidic linkage 3′ to a nucleoside opposite to a target nucleoside. In some embodiments, an internucleotidic linkage at position +5 relative to a nucleoside opposite to a target nucleoside (e.g., for . . . N₊₅N₊₄N₊₃N₊₂N₊₁N₀ . . . , the internucleotidic linkage linking N₊₄ and N₊₅ wherein N₀ is a nucleoside opposite to a target nucleoside) is not a Rp phosphorothioate internucleotidic linkage. In some embodiments, an internucleotidic linkage at positions +11 is not a Sp phosphorothioate internucleotidic linkage. In some embodiments, one or more or all internucleotidic linkages at positions +6 to +8 relative to a nucleoside opposite to a target nucleoside are each independently a modified internucleotidic linkage, optionally chirally controlled. In some embodiments, each of them is independently a phosphorothioate internucleotidic linkage. In some embodiments, each of them is independently a Sp phosphorothioate internucleotidic linkage. In some embodiments, one or more or all internucleotidic linkages at positions +6 to +8 relative to a nucleoside opposite to a target nucleoside are each independently a phosphorothioate internucleotidic linkage, optionally chirally controlled. In some embodiments, one or more or all internucleotidic linkages at positions +6, +7, +8, +9, and +11 are each independently Rp internucleotidic linkages. In some embodiments, one or more or all internucleotidic linkages at positions +6, +7, +8, +9, and +11 are each independently Rp phosphorothioate internucleotidic linkages. In some embodiments, one or more or all internucleotidic linkages at positions +5, +6, +7, +8, and +9 relative to a nucleoside opposite to a target adenosine are each independently Sp internucleotidic linkages. In some embodiments, one or more or all internucleotidic linkages at positions +5, +6, +7, +8, and +9 relative to a nucleoside opposite to a target adenosine are each independently Sp phosphorothioate internucleotidic linkages. In some embodiments, an internucleotidic linkage at position +5 is a Sp internucleotidic linkage. In some embodiments, an internucleotidic linkage at position +5 is a Sp phosphorothioate internucleotidic linkage. In some embodiments, an internucleotidic linkage at position +6 is a Sp internucleotidic linkage. In some embodiments, an internucleotidic linkage at position +6 is a Sp phosphorothioate internucleotidic linkage. In some embodiments, an internucleotidic linkage at position +7 is a Sp internucleotidic linkage. In some embodiments, an internucleotidic linkage at position +7 is a Sp phosphorothioate internucleotidic linkage. In some embodiments, an internucleotidic linkage at position +8 is a Sp internucleotidic linkage. In some embodiments, an internucleotidic linkage at position +8 is a Sp phosphorothioate internucleotidic linkage. In some embodiments, an internucleotidic linkage at position +9 is a Sp internucleotidic linkage. In some embodiments, an internucleotidic linkage at position +9 is a Sp phosphorothioate internucleotidic linkage. In some embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently chirally controlled and a Sp internucleotidic linkage. In some embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each independently chirally controlled and are Sp. In some embodiments, each phosphorothioate internucleotidic linkages 5′ to a nucleoside opposite to a target nucleoside (e.g., a target adenosine) is chirally controlled. In some embodiments, each phosphorothioate internucleotidic linkages 5′ to a nucleoside opposite to a target nucleoside (e.g., a target adenosine) is Sp.

Alternatively or additionally, as described herein (e.g., illustrated in certain Examples), in some embodiments, about 5%-90%, about 10-80%, about 10-75%, about 10-70%, 10%-60%, 10-50%, 10-40%, 10-30%, 15-40%, 20-30%, 25-30%, or about or at least about 5%, 10%, 15%, 20%, 25%, 30%, 35%, 40%, 45%, or 50%, of all internucleotidic linkages in an oligonucleotide are independently a natural phosphate linkage. In some embodiments, about 5%-90%, about 10-80%, about 10-75%, about 10-70%, 10%-60%, 10-50%, 10-40%, 10-30%, 15-40%, 20-30%, 25-30%, or about or at least about 5%, 10%, 15%, 20%, 25%, 30%, 35%, 40%, 45%, or 50%, of all internucleotidic linkages 5′ to a nucleoside opposite to a target nucleoside (e.g., a target adenosine) are independently a natural phosphate linkage. In some embodiments, one or more, e.g., about 1-15, 1-10, 1-9, 1-8, 1-7, 1-6, 1-5, 5-6, 5-7, 5-8, 5-9, 5-10, or about or at least about 1, 2, 3, 4, 5, 6, 7, 8, 9, or 10, internucleotidic linkages in an oligonucleotide are independently a natural phosphate linkage. In some embodiments, one or more, e.g., about 1-15, 1-10, 1-9, 1-8, 1-7, 1-6, 1-5, 5-6, 5-7, 5-8, 5-9, 5-10, or about or at least about 1, 2, 3, 4, 5, 6, 7, 8, 9, or 10, internucleotidic linkages 5′ to a nucleoside opposite to a target nucleoside (e.g., a target adenosine) are independently a natural phosphate linkage. In some embodiments, one or more internucleotidic linkages at one or more of positions +3 (between N₊₄N+₃), +4, +6, +8, +9, +12, +14, +15, +17, and +18 are independently a natural phosphate linkage. In some embodiments, there are 4 natural phosphate linkages 5′ to a nucleoside opposite to a target nucleoside. In some embodiments, there are 5 natural phosphate linkages 5′ to a nucleoside opposite to a target nucleoside. In some embodiments, there are 6 natural phosphate linkages 5′ to a nucleoside opposite to a target nucleoside. In some embodiments, there are 7 natural phosphate linkages 5′ to a nucleoside opposite to a target nucleoside. In some embodiments, there are 8 natural phosphate linkages 5′ to a nucleoside opposite to a target nucleoside. In some embodiments, there are 9 natural phosphate linkages 5′ to a nucleoside opposite to a target nucleoside. In some embodiments, there are 10 natural phosphate linkages 5′ to a nucleoside opposite to a target nucleoside. In some embodiments, one or more internucleotidic linkages 3′ to a nucleoside opposite to a target nucleoside (e.g., a target adenosine) are each independently a natural phosphate linkage. In some embodiments, there is one natural phosphate linkage 3′ to a nucleoside opposite to a target nucleoside. In some embodiments, an internucleotidic linkage at position -3 is a natural phosphate linkage.

Alternatively or additionally, as described herein (e.g., illustrated in certain Examples), in some embodiments, about 5%-90%, about 10-80%, about 10-75%, about 10-70%, 10%-60%, 10-50%, 10-40%, 10-30%, 15-40%,20-30%, 25-30%, 30%-70%, ₄₀-70%, 40%-65%, 40%-60%, or about or at least about 5%, 10%, 15%, 20%, 25%, 30%, 35%, 40%, 45%, 50%, 55%, 60%, or 65% of all internucleotidic linkages in an oligonucleotide are independently a phosphorothioate internucleotidic linkage. In some embodiments, about 5%-90%, about 10-80%, about 10-75%, about 10-70%, 10%-60%, 10-50%, 10-40%, 10-30%, 15-40%, 20-30%, 25-30%, 30%-70%, 40-70%, 40%-65%, 40%-60%, or about or at least about 5%, 10%, 15%, 20%, 25%, 30%, 35%, 40%, 45%, 50%, 55%, 60%, or 65% of all internucleotidic linkages 5′ to a nucleoside opposite to a target nucleoside (e.g., a target adenosine) are independently a natural phosphate linkage. In some embodiments, one or more, e.g., about 1-30, 1-25, 1-20, 1-15, 5-30, 5-25, 5-20, 5-15, 10-30, 10-25, 10-20, 10-15, or about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, internucleotidic linkages in an oligonucleotide are independently a phosphorothioate internucleotidic linkage. In some embodiments, one or more, e.g., about 1-30, 1-25, 1-20, 1-15, 5-30, 5-25, 5-20, 5-15, 10-30, 10-25, 10-20, 10-15, or about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, internucleotidic linkages 5′ to a nucleoside opposite to a target nucleoside (e.g., a target adenosine) are independently a phosphorothioate internucleotidic linkage. In some embodiments, one or more internucleotidic linkages at one or more of positions +1 (between N+,No), +2, +5, +6, +7, +8, +11, +14, +15, +16, +17, +19, +20, +21, and +22 are independently a phosphorothioate internucleotidic linkage. In some embodiments, there are 5 or more phosphorothioate internucleotidic linkages 5′ to a nucleoside opposite to a target nucleoside. In some embodiments, there are 10 or more phosphorothioate internucleotidic linkages 5′ to a nucleoside opposite to a target nucleoside. In some embodiments, there are 11 or more phosphorothioate internucleotidic linkages 5′ to a nucleoside opposite to a target nucleoside. In some embodiments, there are 12 or more phosphorothioate internucleotidic linkages 5′ to a nucleoside opposite to a target nucleoside. In some embodiments, there are 13 or more phosphorothioate internucleotidic linkages 5′ to a nucleoside opposite to a target nucleoside. In some embodiments, there are 14 or more phosphorothioate internucleotidic linkages 5′ to a nucleoside opposite to a target nucleoside. In some embodiments, there are 15 or more phosphorothioate internucleotidic linkages 5′ to a nucleoside opposite to a target nucleoside. In some embodiments, one or more internucleotidic linkages 3′ to a nucleoside opposite to a target nucleoside (e.g., a target adenosine) are each independently a phosphorothioate internucleotidic linkage. In some embodiments, there is one phosphorothioate internucleotidic linkage 3′ to a nucleoside opposite to a target nucleoside. In some embodiments, there are two phosphorothioate internucleotidic linkages 3′ to a nucleoside opposite to a target nucleoside. In some embodiments, there are three phosphorothioate internucleotidic linkages 3′ to a nucleoside opposite to a target nucleoside. In some embodiments, one or more or all internucleotidic linkages at positions −1, −4 and −5 are independently a phosphorothioate internucleotidic linkage. In some embodiments, each phosphorothioate internucleotidic linkage is independently chirally controlled. In some embodiments, about or at least about 80%, 85%, 90% or 95% of all phosphorothioate internucleotidic linkages are independently Sp. In some embodiments, each phosphorothioate internucleotidic linkage is independently Sp.

Alternatively or additionally, as described herein (e.g., illustrated in certain Examples), in some embodiments, about 5%-90%, about 10-80%, about 10-75%, about 10-70%, 10%-60%, 10-50%, 10-40%, 10-30%, 10%-20%, 10-15%, 15-40%, 15%-35%, 15%-30%, 15-25%, 15-20%, or about or at least about 5%, 10%, 15%, 20%, 25%, 30%, 35%, 40%, 45%, or 50%, of all internucleotidic linkages in an oligonucleotide are independently anon-negatively charged internucleotidic linkage. In some embodiments, about 5%-90%, about 10-80%, about 10-75%, about 10-70%, 10%-60%, 10-50%, 10-40%, 10-30%, 10%-20%, 10-15%, 15-40%, 15%-35%, 15%-30%, 15-25%, 15-20%, or about or at least about 5%, 10%, 15%, 20%, 25%, 30%, 35%, 40%, 45%, or 50%, of all internucleotidic linkages 5′ to a nucleoside opposite to a target nucleoside (e.g., a target adenosine) are each independently a non-negatively charged internucleotidic linkage. In some embodiments, one or more, e.g., about 1-15, 1-10, 1-9, 1-8, 1-7, 1-6, 1-5, 5-6, 5-7, 5-8, 5-9, 5-10, or about or at least about 1, 2, 3, 4, 5, 6, 7, 8, 9, or 10, internucleotidic linkages in an oligonucleotide is independently a non-negatively charged internucleotidic linkage. In some embodiments, one or more, e.g., about 1-15, 1-10, 1-9, 1-8, 1-7, 1-6, 1-5, 5-6, 5-7, 5-8, 5-9, 5-10, or about or at least about 1, 2, 3, 4, 5, 6, 7, 8, 9, or 10, internucleotidic linkages 5′ to a nucleoside opposite to a target nucleoside (e.g., a target adenosine) is independently a non-negatively charged internucleotidic linkage. In some embodiments, one or more internucleotidic linkages at one or more or all of positions +5 (between N₊₅N₊₄), +10, +13 or +23 are independently a non-negatively charged internucleotidic linkage. In some embodiments, there are 2 or more non-negatively charged internucleotidic linkages 5′ to a nucleoside opposite to a target nucleoside. In some embodiments, there are 3 or more non-negatively charged internucleotidic linkages 5′ to a nucleoside opposite to a target nucleoside. In some embodiments, there are 4 or more non-negatively charged internucleotidic linkages 5′ to a nucleoside opposite to a target nucleoside. In some embodiments, there are 5 or more non-negatively charged internucleotidic linkages 5′ to a nucleoside opposite to a target nucleoside. In some embodiments, one or more internucleotidic linkages 3′ to a nucleoside opposite to a target nucleoside (e.g., a target adenosine) are each independently a non-negatively charged internucleotidic linkage. In some embodiments, there is one non-negatively charged internucleotidic linkage 3′ to a nucleoside opposite to a target nucleoside. In some embodiments, there are two or more non-negatively charged internucleotidic linkages 3′ to a nucleoside opposite to a target nucleoside. In some embodiments, there are two non-negatively charged internucleotidic linkages 3′ to a nucleoside opposite to a target nucleoside. In some embodiments, one or both internucleotidic linkages at positions −2 and −6 are independently a non-negatively charged internucleotidic linkage. In some embodiments, a non-negatively charged internucleotidic linkage is a neutral internucleotidic linkage. In some embodiments, a non-negatively charged internucleotidic linkage is independently a neutral internucleotidic linkage. In some embodiments, a non-negatively charged internucleotidic linkage is a phosphoryl guanidine internucleotidic linkage. In some embodiments, each non-negatively charged internucleotidic linkage is independently a phosphoryl guanidine internucleotidic linkage. In some embodiments, a non-negatively charged internucleotidic linkage is n001. In some embodiments, each non-negatively charged internucleotidic linkage is independently n001. In some embodiments, a non-negatively charged internucleotidic linkage is chirally controlled. In some embodiments, each non-negatively charged internucleotidic linkage is independently chirally controlled. In some embodiments, a non-negatively charged internucleotidic linkage is Rp. In some embodiments, a non-negatively charged internucleotidic linkage is Sp. In some embodiments, each non-negatively charged internucleotidic linkage is independently Sp. In some embodiments, each n001 is independently Sp except that each n001 bonded to 3′-carbon of dI is independently Rp.

### ADAR

Among other things, provided technologies can provide modification/editing of target adenosine by converting A to I. In some embodiments, oligonucleotides and/or duplexes formed by oligonucleotides with target nucleic acids interact with proteins, e.g., ADAR proteins. In some embodiments, such proteins comprise adenosine modifying activities and can modify target adenosine in target nucleic acids, e.g., converting them to inosine.

ADAR proteins are naturally expressed proteins in various cells, tissues, organs and/or organism. It has been reported that some ADAR proteins, e.g., ADAR1 and ADAR2, can edit adenosine through deamination, converting adenosine to inosine which can provide a number of functions including being read as or similar to G during translation. Mechanism of ADAR-mediated mRNA editing (e.g., deamination) has been reported. For example, ADAR proteins are reported to catalyze conversion of adenosine to inosine on double-stranded RNA substrates with mismatches. As appreciated by those skilled in the art, inosine can be recognized as guanosine by cellular translation and/or splicing machinery. ADAR can thus be used for functional adenosine to guanosine editing of nucleic acids, e.g., pre-mRNA and mRNA substrates.

In some embodiments, the present disclosure provides oligonucleotides and compositions thereof for ADAR-mediated editing of target adenosine in target nucleic acids, e.g. RNA. ADAR-mediated RNA-editing can offer several advantages over DNA-editing, e.g., delivery is simplified as expression of recombinant proteins like Cas9 is not required. Both ADAR1 and ADAR2 are endogenous enzymes, so cellular delivery of oligonucleotides alone can be sufficient for editing. Off-target effects, if any, are transient and changes are not made to genomic DNA. Additionally, ADAR-mediated editing can be used in post-mitotic cells and it does not require an HDR-template for repair. Three vertebrate ADAR genes have been reported with common functional domains (Nishikura Nat Rev Mol Cell Biol. 2016 February; 17(2): 83-96.; Nishikura Annu Rev Biochem. 2010; 79: 321-349.; Thomas and Beal Bioessays. 2017 April; 39(4)). All 3 ADARs contain a dsRNA-binding domains (dsRBD), which can contact dsRNA substrates. Some ADAR1 also contains Z-DNA-binding domains. ADAR1 has been reported to expressed significantly in brain, lung, kidney, liver, and heart, etc., and may occur in two isoforms. In some embodiments, isoform p150 can be induced by interferon while isoform p110 can be constitutively expressed. In some embodiments, it can be beneficial to utilize p110 as it is reported to be ubiquitously and constitutively expressed. ADAR2 can be highly expressed, e.g. in the brain and lungs, and is reported to be exclusively localized to the nucleus. ADAR3 is reported to be catalytically inactive and expressed only in the brain. Potential differences in tissue expression can be taken into consideration when choosing a therapeutic target.

Use of oligonucleotides for RNA editing by ADAR has been reported. Among other things, the present disclosure recognizes that previously reported technologies generally suffer one or more disadvantages, such as low stability (e.g., oligonucleotides with natural RNA sugars), low editing efficiency, low editing specificity (e.g., a number of As are edited in a portion of a target nucleic acid substantially complementary to an oligonucleotide), specific structures in oligonucleotides for ADAR recognition/recruitment, exogenous proteins (e.g., those engineered to recognize oligonucleotides with specific structures and/or duplexes thereof (e.g., with target nucleic acids) for editing), etc. Additionally, previously reported technologies typically utilize stereorandom oligonucleotide compositions when oligonucleotides comprise one or more chiral linkage phosphorus of modified internucleotidic linkages.

For example, various reported oligonucleotides contain ADAR-recruiting domains. Merkle et al., Nat Biotechnol. 2019 February; 37(2):133-138disclosed oligonucleotides comprising an imperfect 20-bp hairpin ADAR-recruiting domain that is an intramolecular stem loop to recruit endogenous human ADAR2 to edit endogenous transcript. Oligonucleotides reported in Mali et al., Nat Methods. 2019 March; 16(3):239-242contain ADAR substrate GluR2 pre-messenger RNA sequences or MS2 hairpins in addition to specificity domains that hybridize to the target mRNA.

Certain reported editing approach utilizes exogenous or engineered proteins, e.g., those utilizing CRISPR/Cas9 system. For example, Komor et al. Nature 2016 volume533, pages420-424 disclosed deaminase coupled with CRISPR-Cas9 to create programmable DNA base editors. Since it engages in exogenous editing proteins, it requires the delivery of both the CRISPR/Cas9 system and the guide RNA.

Among other things, the present disclosure provides technologies comprising one or more features such as sugar modifications, base modifications, internucleotidic linkage modifications, control of stereochemistry, various patterns thereof, etc. to solve one or more or all disadvantaged suffered from prior adenosine editing technologies, for example, through providing chirally controlled oligonucleotide compositions of designed oligonucleotides described herein. For example, ADAR-recruiting loops are optional and not required for provided technology.

As appreciated by those skilled in the art, one or more of such useful features may be utilized to improve oligonucleotides in prior technologies (e.g., those described in WO 2016097212, WO 2017220751, WO 2018041973, WO 2018134301, oligonucleotides and oligonucleotide compositions of each of which are independently incorporated by reference). In some embodiments, the present disclosure provides improvements of prior technologies by apply one or more useful features described herein to prior reported oligonucleotide base sequences. In some embodiments, the present disclosure provides chirally controlled oligonucleotide compositions of previously reported oligonucleotides that may be useful for adenosine editing. In some embodiments, the present disclosure provides improvements of previously reported adenosine editing using stereorandom oligonucleotide compositions by performing such editing using chirally controlled oligonucleotide compositions.

As reported, ADAR proteins may have various isoforms. For example, ADAR1 has, among others, a reported p110 isoform and a reported p150 isoform. In some embodiments, certain chirally controlled oligonucleotide compositions can provide high levels of adenosine modification (e.g., conversion of A to I) with multiple isoforms, in some embodiments, both p 110 and p150 isoforms, while stereorandom compositions provide low levels of adenosine modification for one or more isoforms (e.g., p110). In some embodiments, chirally controlled oligonucleotide composition are particularly useful for adenosine modification in systems (e.g., cells, tissues, organs, organisms, subjects, etc.) expressing or comprising the p110 isoform of ADAR1, particularly those expressing or comprising high levels of the p110 isoform of ADAR1 relative to the p150 isoform, or those expressing no or low levels of ADAR1 p150.

In some embodiments, the present disclosure provides Cis-acting (CisA) oligonucleotide that do not require stem loop in the structure. In some embodiments, a provided oligonucleotide can form a dsRNA structure with a target mRNA through base pairing. In some embodiments, formed dsRNA structures (optionally with secondary mismatches) contain bulges that promote ADAR binding and therefore, can facilitate ADAR-mediated editing (e.g., deamination of a target adenosine). In some embodiments, oligonucleotides of the present disclosure are shorter than LSL oligonucleotides or CSL oligonucleotides, e.g., no more than or about 32 nt, no more than or about 31 nt, no more than or about 30 nt, no more than or about 29 nt, no more than or about 28 nt, no more than or about 27 nt, or no more than or about 26 nt in length, and can provide high editing efficiency.

### Duplexing and Targeting Regions

In some embodiments, the present disclosure provides an oligonucleotide comprising:
a duplexing region; and
a targeting region;
   wherein:
a duplexing region is capable of forming a duplex with a nucleic acid; and
a targeting region is capable of forming a duplex with a target nucleic acid comprising a target adenosine.

In some embodiments, a duplexing region is or comprises a first domain as described herein. In some embodiments, a targeting region is or comprises a second domain as described herein.

In some embodiments, a duplexing region is capable of forming a duplex with a nucleic acid, wherein the nucleic acid is not a target nucleic acid. In some embodiments, a duplexing region forms a duplex with a target nucleic acid. In some embodiments, a duplexing region forms a duplex with a nucleic acid expressed in a system, e.g., a cell. In some embodiments, a duplexing region forms a duplex with an exogenous nucleic acid, e.g., an oligonucleotide. In some embodiments, a duplexing region forms a duplex with a nucleic acid which is or comprises a RNA portion. In some embodiments, a duplex formed can be recognized by a polypeptide such as an ADAR polypeptide, e.g., ADAR1 (p110 or p150 or both), ADAR2, etc. In some embodiments, a duplex formed can recruit a polypeptide such as an ADAR polypeptide, e.g., ADAR1 (p110 or p150 or both), ADAR2, etc. In some embodiments, a duplex formed recruit ADAR1. In some embodiments, a duplex formed recruit ADAR1 p110. In some embodiments, a duplex formed recruit ADAR1 p150. In some embodiments, a duplex formed recruit ADAR2. In some embodiments, a duplex formed recruit ADAR1 p110 and p150. In some embodiments, a duplex formed recruit ADAR1 and ADAR2. In some embodiments, a duplex formed recruit ADAR1 p110, ADAR p150 and/or ADAR2. In some embodiments, a duplex formed recruit ADAR1 p110 and p150 and ADAR2.

In some embodiments, a duplexing region forms a duplex with an oligonucleotide (which oligonucleotide may be referred to as “a duplexing oligonucleotide”). In some embodiments, a duplexing oligonucleotide comprises one or more modified nucleobases, modified sugars and/or modified internucleotidic linkages. In some embodiments, an duplexing oligonucleotide comprises a duplex-forming region that is complementary to a duplexing region. As those skilled in the art appreciate, in many instances, perfect complementary is not required and one or more wobbles, bulges, mismatches, etc. may be well tolerated. For example, ADAR proteins have been reported to bind to and/or utilize as substrates both perfectly and imperfectly complementary duplexes.

Duplexing regions and/or duplexing-forming regions can be of various lengths. In some embodiments, they are at least 10 (e.g., about or at least about 10, 11, 12, 13, 14, 15, 16, 17, 18, 19, 20, 21, 22, 23, 24, 25, 26, 27, 28, 29 or 30 or more, about 10-20, 10-25, 10-30, 10-40, 10-50, 10-100, 14-20, 14-25, 14-30, 14-40, 14-50, 14-100, 15-20, 15-25, 15-30, 15-40, 15-50, 15-100, 16-20, 16-25, 16-30, 16-40, 16-50, 16-100, 17-20, 17-25, 17-30, 17-40, 17-50, 17-100, 18-20, 18-25, 18-30, 18-40, 18-50, 18-100, 19-20, 19-25, 19-30, 19-40, 19-50, 19-100, 20-25, 20-30, 20-40, 20-50, 20-100, etc.) nucleosides in length. In some embodiments, a length is about or at least about 10 nucleosides in length. In some embodiments, a length is about or at least about 11 nucleosides in length. In some embodiments, a length is about or at least about 12 nucleosides in length. In some embodiments, a length is about or at least about 13 nucleosides in length. In some embodiments, a length is about or at least about 14 nucleosides in length. In some embodiments, a length is about or at least about 15 nucleosides in length. In some embodiments, a length is about or at least about 16 nucleosides in length. In some embodiments, a length is about or at least about 17 nucleosides in length. In some embodiments, a length is about or at least about 18 nucleosides in length. In some embodiments, a length is about or at least about 19 nucleosides in length. In some embodiments, a length is about or at least about 20 nucleosides in length.

In some embodiments, a duplexing oligonucleotide consists of or consists essentially of a duplex-forming region. In some embodiments, a duplexing oligonucleotide further comprises one or more additional regions in addition to a duplex-forming region. In some embodiments, a duplexing oligonucleotide comprises a stem-loop region. In some embodiments, a duplexing oligonucleotide comprises or consists of a duplex-forming region and a stem-loop region. In some embodiments, a stem region is about or at least about 2, 3, 4, 5, 6, 7, 8, 9, or 10 (e.g., about or at least about 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more, about 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, 8-50, 9-10, 9-15, 9-20, 9-25, 9-30, 9-40, 9-50, 10-15, 10-25, 10-30, 10-40, 10-50, 10-100, etc.) nucleobase in length. In some embodiments, it is about or at least about 5 nucleobases in length. In some embodiments, it is about or at least about 6 nucleobases in length. In some embodiments, it is about or at least about 7 nucleobases in length. In some embodiments, it is about or at least about 8 nucleobases in length. In some embodiments, it is about or at least about 9 nucleobases in length. In some embodiments, it is about or at least about 10 nucleobases in length.

In some embodiments, one or more additional regions may promote, encourage, facilitate and/or contribute recruitment of and/or recognition by and/or interaction with a polypeptide, e.g., ADAR1 (p110 and/or p150) and/or ADAR2. In some embodiments, for duplexing oligonucleotides comprising one or more additional regions, shorter duplex-forming regions may be utilized compared to absence of such additional regions.

In some embodiments, a duplex structure formed by a duplex region and a duplexing oligonucleotide can recruit a polypeptide, e.g., ADAR1 (p110 and/or p150) and/or ADAR2. In some embodiments, a duplex structure is or comprises a recruiting portion as described in WO 2016/097212.

In some embodiments, a duplexing oligonucleotide comprises one or more sugar, nucleobase, and/or internucleotidic linkage modifications as described herein. In some embodiments, a duplexing oligonucleotide comprises one or more sugar modification. In some embodiments, a majority, as described herein, of, or all of, the sugars in a duplexing oligonucleotide is a modified sugar. In some embodiments, a modified sugar is a 2′-modified sugar. In some embodiments, each modified sugar is independently a 2′-modified sugar. In some embodiments, each modified sugar is independently selected from a 2′-F modified sugar, a bicyclic sugar, or a 2′-OR modified sugar wherein R is not hydrogen. In some embodiments, each modified sugar is independently selected from a 2′-F modified sugar, a bicyclic sugar, or a 2′-OR modified sugar wherein R is optionally substituted C₁₋₆ aliphatic. In some embodiments, each modified sugar is independently selected from a 2′-F modified sugar or a 2′-OR modified sugar wherein R is optionally substituted C₁₋₆ aliphatic. In some embodiments, each 2′-OR modified sugar is independently a 2′-OMe or a 2′-MOE modified sugar. In some embodiments, each 2′-OR modified sugar is independently a 2′-OMe modified sugar. In some embodiments, each 2′-OR modified sugar is independently a 2′-MOE modified sugar. In some embodiments, each 2′-OR modified sugar is independently a 2′-F modified sugar. In some embodiments, a duplexing oligonucleotide comprises one or more modified internucleotidic linkages, e.g., phosphorothioate internucleotidic linkages. In some embodiments, a majority, as described herein, of or all of internucleotidic linkages of a duplexing oligonucleotide are independently modified internucleotidic linkages. In some embodiments, each internucleotidic linkage of a duplexing oligonucleotide is independently a modified internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral internucleotidic linkage. In some embodiments, a modified internucleotidic linkage is a phosphoryl guanidine internucleotidic linkage. In some embodiments, a modified internucleotidic linkage is n001. In some embodiments, each modified internucleotidic linkage is a phosphorothioate internucleotidic linkage. In some embodiments, each internucleotidic linkage is a phosphorothioate internucleotidic linkage. In some embodiments, a phosphorothioate internucleotidic linkage is chirally controlled. In some embodiments, a phosphorothioate internucleotidic linkage is not chirally controlled. In some embodiments, a majority, as described herein, of, or all of, chirally controlled phosphorothioate internucleotidic linkages are independently Sp. In some embodiments, all phosphorothioate internucleotidic linkages are Sp. In some embodiments, a duplexing oligonucleotide comprises one or more (e.g., 1, 2, 3, 4, 5, 6, 7, 8, 9, 10, 11, 12, 13, 14, 15, 16, 17, 18, 19 or 20 or more) natural phosphate linkages. In some embodiments, when an oligonucleotide comprises one or more natural phosphate linkages, one or several internucleotidic linkages at the 5′ and/or 3′ end are independently modified internucleotidic linkages as described herein. In some embodiments, several internucleotidic linkages at both the 5′ and 3′ ends are independently modified internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 5′ end are modified internucleotidic linkages as described herein, e.g., phosphorothioate internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 3′ end are modified internucleotidic linkages as described herein, e.g., phosphorothioate internucleotidic linkages. In some embodiments, increasing the number of modified internucleotidic linkages, e.g., phosphorothioate internucleotidic linkages, etc., can increase editing efficiency, e.g., when more natural DNA/RNA sugars, 2′-F modified sugars, etc., are bonded to modified internucleotidic linkages such as phosphorothioate internucleotidic linkages.

In some embodiments, a duplexing region comprises one or more sugar, nucleobase and/or internucleotidic linkage modifications as described herein. In some embodiments, a duplexing region comprises one or more (e.g., 1-30, 1-20, 1-10, 1, 2, 3, 4, 5, 6, 7, 8, 9, 10, 11, 12, 13, 14, 15, 16, 17, 18, 19, 20 or more, etc.) modified sugars as described herein. In some embodiments, a majority, as described herein, of or all of sugars in a duplexing region are each independently a modified sugar as described herein. In some embodiments, a modified sugar is a 2′-modified sugar. In some embodiments, each modified sugar is independently a 2′-modified sugar. In some embodiments, each modified sugar is independently selected from a 2′-F modified sugar, a bicyclic sugar, or a 2′-OR modified sugar wherein R is not hydrogen. In some embodiments, each modified sugar is independently selected from a 2′-F modified sugar, a bicyclic sugar, or a 2′-OR modified sugar wherein R is optionally substituted C₁₋₆ aliphatic. In some embodiments, each modified sugar is independently selected from a 2′-F modified sugar or a 2′-OR modified sugar wherein R is optionally substituted C₁₋₆ aliphatic. In some embodiments, each 2′-OR modified sugar is independently a 2′-OMe or a 2′-MOE modified sugar. In some embodiments, each 2′-OR modified sugar is independently a 2′-OMe modified sugar. In some embodiments, each 2′-OR modified sugar is independently a 2′-MOE modified sugar. In some embodiments, each 2′-OR modified sugar is independently a 2′-F modified sugar. In some embodiments, about 50%-100%, 60%-100%, 70%-100%, 50%-90%, 50%-80%, 60%-90%, 60%-80%, 70%-90%, 70%-80%, or about or at least about 60%, 70%, 75%, 80%, 85%, 90%, 95% or more of sugars in a duplexing region are each independently a 2′-F modified sugar. In some embodiments, as described herein, one or more sugars at an end of an oligonucleotide is independently a modified sugar. In some embodiments, as described herein, one or more sugars at an end of an oligonucleotide is independently a bicyclic sugar or a 2′-OR modified sugar wherein R is C₁₋₆ aliphatic. In some embodiments, as described herein, one or more (e.g., 1, 2, 3, 4, 5, 6, 7, 8, 9, 10, 2-20, 2-10, 2-5, 3-5, etc.) sugars at an end of an oligonucleotide are each independently a 2′-OR modified sugar wherein R is C₁₋₆ aliphatic. In some embodiments, one or more (e.g., 1, 2, 3, 4, 5, 6, 7, 8, 9, 10, 2-20, 2-10, 2-5, 3-5, etc.) sugars at both ends of an oligonucleotide are each independently a modified sugar; for example, in some oligonucleotides, 3 or more sugars at the 5′ end are 2′-OMe modified sugars, and 4 or more sugars at the 3′ end are 2′-OMe modified sugars. In some embodiments, a duplexing region comprises one or more (e.g., 1-30, 1-20, 1-10, 1, 2, 3, 4, 5, 6, 7, 8, 9, 10, 11, 12, 13, 14, 15, 16, 17, 18, 19, 20 or more, etc.) modified internucleotidic linkages as described herein. In some embodiments, one or more (e.g.. 1, 2, 3, 4, 5, 6, 7, 8, 9, 10, 2-20, 2-10, 2-5, 3-5, etc.) internucleotidic linkages at the 5′ and/or 3′ ends of an oligonucleotide are each independently a modified internucleotidic linkage, e.g., in some embodiments, each independently selected from a non-negatively charged internucleotidic linkage, a neutral internucleotidic linkage, a phosphoryl guanidine internucleotidic linkage, n001 and a phosphorothioate internucleotidic linkage. In some embodiments, one or more (e.g., 1, 2, 3, 4, 5, 6, 7, 8, 9, 10, 2-20, 2-10, 2-5, 3-5, etc.) internucleotidic linkages at the 5′ end of an oligonucleotide are each independently a modified internucleotidic linkage, and one or more (e.g., 1, 2, 3, 4, 5, 6, 7, 8, 9, 10, 2-20, 2-10, 2-5, 3-5, etc.) internucleotidic linkages at the 3′ end of an oligonucleotide are each independently a modified internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral internucleotidic linkage. In some embodiments, a modified internucleotidic linkage is a phosphoryl guanidine internucleotidic linkage. In some embodiments, a modified internucleotidic linkage is n001. In some embodiments, each modified internucleotidic linkage is a phosphorothioate internucleotidic linkage. In some embodiments, each internucleotidic linkage is a phosphorothioate internucleotidic linkage. In some embodiments, a phosphorothioate internucleotidic linkage is chirally controlled. In some embodiments, a phosphorothioate internucleotidic linkage is not chirally controlled. In some embodiments, a majority, as described herein, of, or all of, chirally controlled phosphorothioate internucleotidic linkages are independently Sp. In some embodiments, all phosphorothioate internucleotidic linkages are Sp. In some embodiments, chiral modified internucleotidic linkages, e.g., phosphorothioate internucleotidic linkages, are not chirally controlled. In some embodiments, a duplexing region comprises one or more (e.g., 1, 2, 3, 4, 5, 6, 7, 8, 9, 10, 11, 12, 13, 14, 15, 16, 17, 18, 19 or 20 or more) natural phosphate linkages. In some embodiments, when an oligonucleotide comprises one or more natural phosphate linkages, one or several internucleotidic linkages at the 5′ and/or 3′ end are independently modified internucleotidic linkages as described herein. In some embodiments, several internucleotidic linkages at both the 5′ and 3′ ends are independently modified internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 5′ end are modified internucleotidic linkages as described herein, e.g., phosphorothioate internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 3′ end are modified internucleotidic linkages as described herein, e.g., phosphorothioate internucleotidic linkages. In some embodiments, incorporation of one or more (e.g., 1, 2, 3, 4, 5, 6, 7, 8, 9, 10, 2-20, 2-10, 2-5, 3-5, etc.) natural phosphate linkages at duplexing regions increase editing efficiency. In some embodiments, a majority of internucleotidic linkages (e.g., 50%-100%, 60%-100%, 70%-100%, 50%-90%, 50%-80%, 60%-90%, 60%-80%, 70%-90%, 70%-80%, or about or at least about 60%, 70%, 75%, 80%, 85%, 90%, 95% or more) in a duplexing region are independently natural phosphate linkages. In some embodiments, except the one or more natural phosphate linkages at an end of an oligonucleotide (if any), each other internucleotidic linkage in a duplexing region is independently a natural phosphate linkage.

In some embodiments, a targeting region is or comprises an editing region as described herein. In some embodiments, a targeting region comprises 5′-N₁N₀N₋₁-3′ as described herein.

In some embodiments, a targeting region comprises one or more sugar, nucleobase and/or internucleotidic linkage modifications as described herein. In some embodiments, a targeting region comprises one or more (e.g., 1-30, 1-20, 1-10, 1, 2, 3, 4, 5, 6, 7, 8, 9, 10, 11, 12, 13, 14, 15, 16, 17, 18, 19, 20 or more, etc.) modified sugars as described herein. In some embodiments, a majority, as described herein, of or all of sugars in a targeting region are each independently a modified sugar as described herein. In some embodiments, a modified sugar is a 2′-modified sugar. In some embodiments, each modified sugar is independently a 2′-modified sugar. In some embodiments, each modified sugar is independently selected from a 2′-F modified sugar, a bicyclic sugar, or a 2′-OR modified sugar wherein R is not hydrogen. In some embodiments, each modified sugar is independently selected from a 2′-F modified sugar, a bicyclic sugar, or a 2′-OR modified sugar wherein R is optionally substituted C₁₋₆ aliphatic. In some embodiments, each modified sugar is independently selected from a 2′-F modified sugar or a 2′-OR modified sugar wherein R is optionally substituted C₁₋₆ aliphatic. In some embodiments, each 2′-OR modified sugar is independently a 2′-OMe or a 2′-MOE modified sugar. In some embodiments, each 2′-OR modified sugar is independently a 2′-OMe modified sugar. In some embodiments, each 2′-OR modified sugar is independently a 2′-MOE modified sugar. In some embodiments, each 2′-OR modified sugar is independently a 2′-F modified sugar. In some embodiments, about 50%-100%, 60%-100%, 70%-100%, 50%-90%, 50%-80%, 60%-90%, 60%-80%, 70%-90%, 70%-80%, or about or at least about 60%, 70%, 75%, 80%, 85%, 90%, 95% or more of sugars in a targeting region are each independently a bicyclic sugar or a 2′-OR modified sugar wherein R is not hydrogen. In some embodiments, about 50%-100%, 60%-100%, 70%-100%, 50%-90%, 50%-80%, 60%-90%, 60%-80%, 70%-90%, 70%-80%, or about or at least about 60%, 70%, 75%, 80%, 85%, 90%, 95% or more of sugars in a targeting region are each independently a bicyclic sugar or a 2′-OR modified sugar wherein R is optionally substituted C₁₋₆ aliphatic. In some embodiments, about 50%-100%, 60%-100%, 70%-100%, 50%-90%, 50%-80%, 60%-90%, 60%-80%, 70%-90%, 70%-80%, or about or at least about 60%0, 70%, 75%, 80%, 85%, 90%, 95% or more of sugars in a targeting region are each independently a 2′-OMe or 2′-MOE modified sugar. In some embodiments, each sugar in a targeting region except the sugars in an editing region is independently a modified sugar as described herein. In some embodiments, each sugar in a targeting region except the sugars in an editing region is independently a bicyclic sugar or a 2′-OR modified sugar wherein R is optionally substituted C₁₋₆ aliphatic. In some embodiments, each sugar in a targeting region except the sugars in an editing region is independently a 2′-OR modified sugar wherein R is optionally substituted C₁₋₆ aliphatic. In some embodiments, each sugar in a targeting region except the sugars in an editing region is independently a 2′-OMe or 2′-MOE modified sugar. In some embodiments, each sugar in a targeting region except the sugars in an editing region is independently a 2′-OMe modified sugar. In some embodiments, an editing region comprises or consists of three nucleosides wherein a nucleoside opposite to a target adenosine is in the middle of the three. In some embodiments, an editing region consists of three nucleosides wherein a nucleoside opposite to a target adenosine is in the middle of the three. In some embodiments, an editing region comprising or consisting of 5′-N₁N₀N₋₁-3′. In some embodiments, as described herein, one or more (e.g., 1, 2, 3, 4, 5, 6, 7, 8, 9, 10, 2-20, 2-10, 2-5, 3-5, etc.) sugars at an end of an oligonucleotide is independently a modified sugar. In some embodiments, as described herein, one or more (e.g., 1, 2, 3, 4, 5, 6, 7, 8, 9, 10, 2-20, 2-10, 2-5, 3-5, etc.) sugars at an end of an oligonucleotide is independently a bicyclic sugar or a 2′-OR modified sugar wherein R is C₁₋₆ aliphatic. In some embodiments, as described herein, one or more (e.g., 1, 2, 3, 4, 5, 6, 7, 8, 9, 10, 2-20, 2-10, 2-5, 3-5, etc.) sugars at an end of an oligonucleotide are each independently a 2′-OR modified sugar wherein R is C1-6 aliphatic. In some embodiments, one or more (e.g., 1, 2, 3, 4, 5, 6, 7, 8, 9, 10, 2-20, 2-10, 2-5, 3-5, etc.) sugars at both ends of an oligonucleotide are each independently a modified sugar; for example, in some oligonucleotides, 3 or more sugars at the 5′ end are 2′-OMe modified sugars, and 4 or more sugars at the 3′ end are 2′-OMe modified sugars. In some embodiments, a targeting region comprises one or more (e.g., 1-30, 1-20, 1-10, 1, 2, 3, 4, 5, 6, 7, 8, 9, 10, 11, 12, 13, 14, 15, 16, 17, 18, 19, 20 or more, etc.) modified internucleotidic linkages as described herein. In some embodiments, one or more (e.g., 1, 2, 3, 4, 5, 6, 7, 8, 9, 10, 2-20, 2-10, 2-5, 3-5, etc.) internucleotidic linkages at the 5′ and/or 3′ ends of an oligonucleotide are each independently a modified internucleotidic linkage, e.g., in some embodiments, each independently selected from a non-negatively charged internucleotidic linkage, a neutral internucleotidic linkage, a phosphoryl guanidine internucleotidic linkage, n001 and a phosphorothioate internucleotidic linkage. In some embodiments, one or more (e.g., 1, 2, 3, 4, 5, 6, 7, 8. 9, 10, 2-20, 2-10, 2-5, 3-5, etc.) internucleotidic linkages at the 5′ end of an oligonucleotide are each independently a modified internucleotidic linkage, and one or more (e.g., 1, 2, 3, 4, 5, 6, 7, 8, 9, 10, 2-20, 2-10, 2-5, 3-5, etc.) internucleotidic linkages at the 3′ end of an oligonucleotide are each independently a modified internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral internucleotidic linkage. In some embodiments, a modified internucleotidic linkage is a phosphoryl guanidine internucleotidic linkage. In some embodiments, a modified internucleotidic linkage is n001. In some embodiments, each modified internucleotidic linkage is a phosphorothioate internucleotidic linkage. In some embodiments, each internucleotidic linkage is a phosphorothioate internucleotidic linkage. In some embodiments, a phosphorothioate internucleotidic linkage is chirally controlled. In some embodiments, a phosphorothioate internucleotidic linkage is not chirally controlled. In some embodiments, a majority, as described herein, of, or all of, chirally controlled phosphorothioate internucleotidic linkages are independently Sp. In some embodiments, all phosphorothioate internucleotidic linkages are Sp. In some embodiments, chiral modified internucleotidic linkages, e.g., phosphorothioate internucleotidic linkages, are not chirally controlled. In some embodiments, a targeting region comprises one or more (e.g., 1, 2, 3, 4, 5, 6, 7, 8, 9, 10, 11, 12, 13, 14, 15, 16, 17, 18, 19 or 20 or more) natural phosphate linkages. In some embodiments, when an oligonucleotide comprises one or more natural phosphate linkages, one or several internucleotidic linkages at the 5′ and/or 3′ end are independently modified internucleotidic linkages as described herein. In some embodiments, several internucleotidic linkages at both the 5′ and 3′ ends are independently modified internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 5′ end are modified internucleotidic linkages as described herein, e.g., phosphorothioate internucleotidic linkages. In some embodiments, one or more (e.g., 1, 2, 3, 4, 5, 6, 7, 8, 9, 10, 2-20, 2-10, 2-5, 3-5, etc.) internucleotidic linkages at the 3′ end are modified internucleotidic linkages as described herein, e.g., phosphorothioate internucleotidic linkages. In some embodiments, incorporation of one or more (e.g., 1, 2, 3, 4, 5, 6, 7, 8, 9, 10, 2-20, 2-10, 2-5, 3-5, etc.) natural phosphate linkages at targeting regions increase editing efficiency. In some embodiments, a majority of internucleotidic linkages (e.g., 50%-100%, 60%-100%, 70%-100%, 50%-90%, 50%-80%, 60%-90%, 60%-80%, 70%-90%, 70%-80%, or about or at least about 60%, 70%, 75%, 80%, 85%, 90%, 95% or more) in a targeting region are independently natural phosphate linkages. In some embodiments, except the one or more natural phosphate linkages at an end of an oligonucleotide (if any), each other internucleotidic linkage in a targeting region is independently a natural phosphate linkage.

In some embodiments, a targeting region is complementary to a sequence in a target nucleic acid. In some embodiments, a nucleic acid is or comprise RNA. In some embodiments, a nucleic acid is RNA. In some embodiments, a sequence in a target nucleic acid to which a target region is complementary to comprises a target adenosine. As those skilled in the art appreciate, full complementarity in many instances are not required, and one or more wobbles, bulges, mismatches, etc. may be present.

Targeting regions can be of various lengths. In some embodiments, a targeting region is at least 10 (e.g., about or at least about 10, 11, 12, 13, 14, 15, 16, 17, 18, 19, 20, 21, 22, 23, 24, 25, 26, 27, 28, 29 or 30 or more, about 10-20, 10-25, 10-30, 10-40, 10-50, 10-100, 14-20, 14-25, 14-30, 14-40, 14-50, 14-100, 15-20, 15-25, 15-30, 15-40, 15-50, 15-100, 16-20, 16-25, 16-30, 16-40, 16-50, 16-100, 17-20, 17-25, 17-30, 17-40, 17-50, 17-100, 18-20, 18-25, 18-30, 18-40, 18-50, 18-100, 19-20, 19-25, 19-30, 19-40, 19-50, 19-100, 20-25, 20-30, 20-40, 20-50, 20-100, etc.) nucleosides in length. In some embodiments, a length is about or at least about 10 nucleosides in length. In some embodiments, a length is about or at least about 11 nucleosides in length. In some embodiments, a length is about or at least about 12 nucleosides in length. In some embodiments, a length is about or at least about 13 nucleosides in length. In some embodiments, a length is about or at least about 14 nucleosides in length. In some embodiments, a length is about or at least about 15 nucleosides in length. In some embodiments, a length is about or at least about 16 nucleosides in length. In some embodiments, a length is about or at least about 17 nucleosides in length. In some embodiments, a length is about or at least about 18 nucleosides in length. In some embodiments, a length is about or at least about 19 nucleosides in length. In some embodiments, a length is about or at least about 20 nucleosides in length. In some embodiments, a length is about or at least about 21 nucleosides in length. In some embodiments, a length is about or at least about 22 nucleosides in length. In some embodiments, a length is about or at least about 23 nucleosides in length. In some embodiments, a length is about or at least about 24 nucleosides in length. In some embodiments, a length is about or at least about 25 nucleosides in length.

In some embodiments, an oligonucleotide comprises a targeting region and a duplexing region, wherein the targeting region is at the 3′ side of the duplexing region. In some embodiments, an oligonucleotide comprises a targeting region and a duplexing region, wherein the targeting region is at the 5′ side of the duplexing region. In some embodiments, an oligonucleotide consists of a targeting region and a duplexing region, wherein the targeting region is at the 3′ side of the duplexing region. In some embodiments, an oligonucleotide consists of a targeting region and a duplexing region, wherein the targeting region is at the 5′ side of the duplexing region. In some embodiments, an oligonucleotide comprise a targeting region, a duplexing region and a linker region between the target and duplexing regions. In some embodiments, a linker region comprises or is an oligonucleotide moiety.

In some embodiments, oligonucleotides comprising duplexing and targeting regions form complexes including duplexes with other nucleic acids e.g., duplexing oligonucleotides. In some embodiments, the present disclosure provides duplexes comprising oligonucleotides comprising duplexing and targeting regions and nucleic acids that form duplexes with duplexing regions. In some embodiments, the present disclosure provides duplexes comprising oligonucleotides comprising duplexing and targeting regions and duplexing oligonucleotides. In some embodiments, chirally controlled oligonucleotide compositions of oligonucleotides comprising duplexing and targeting regions are utilized. In some embodiments, non-chirally controlled oligonucleotide compositions of oligonucleotides comprising duplexing and targeting regions are utilized. In some embodiments, chirally controlled oligonucleotide compositions of duplexing oligonucleotides are utilized. In some embodiments, non-chirally controlled oligonucleotide compositions of duplexing oligonucleotides are utilized.

In some embodiments, duplexes are formed before administration. In some embodiments, oligonucleotides comprising duplexing and targeting regions and nucleic acids forming duplexes therewith (which may be referred to as “duplexing nucleic acids”) are administered separately. In some embodiments, oligonucleotides comprising duplexing and targeting regions are administered prior to, concurrently with (either in a single or multiple compositions) or subsequently to duplexing nucleic acids (e.g., various duplexing oligonucleotides described herein). In some embodiments, duplexing nucleic acids are present in and/or can be expressed in cells and thus may not need to be administered directly.

In some embodiments, a target nucleic acid is or comprises RNA. In some embodiments, a target nucleic acid is or comprises mRNA. In some embodiments, a target adenosine in a target nucleic acid is edited to I.

### Production of Oligonucleotides and Compositions

Various methods can be utilized for production of oligonucleotides and compositions and can be utilized in accordance with the present disclosure. For example, traditional phosphoramidite chemistry (e.g., phosphoramidites comprising —CH₂CH₂CN and —N(i-Pr)₂) can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the reagents and methods of each of which is incorporated herein by reference.

In some embodiments, chirally controlled/stereoselective preparation of oligonucleotides and compositions thereof comprise utilization of a chiral auxiliary, e.g., as part of monomers, dimers (e.g., chirally pure dimers from separation), monomeric phosphoramidites, dimeric phosphoramidites (e.g., chirally pure dimers from separation), etc. Examples of such chiral auxiliary reagents, monomers, dimers, and phosphoramidites are described in U.S. Pat. No. 9,982,257, US 20170037399, US 20180216108, US 20180216107, U.S. Pat. No. 9,598,458, WO 2017/062862. WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the chiral auxiliary reagents, monomers, dimers, and phosphoramidites of each of which are independently incorporated herein by reference. In some embodiments, a chiral auxiliary is a chiral auxiliary described in any of WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the chiral auxiliaries of each of which are independently incorporated herein by reference.

In some embodiments, chirally controlled preparation technologies, including oligonucleotide synthesis cycles, reagents and conditions are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, and/WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the oligonucleotide synthesis methods, cycles, reagents and conditions of each of which are independently incorporated herein by reference.

Once synthesized, provided oligonucleotides and compositions are typically further purified. Suitable purification technologies are widely known and practiced by those skilled in the art, including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862. WO 2018/067973, WO 2017/160741, WO 2017/192679, WO 2017/210647, WO 2018/098264, WO 2018/223056, or WO 2018/237194 and references cited therein.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

In some embodiments, a useful chiral auxiliary has the structure of

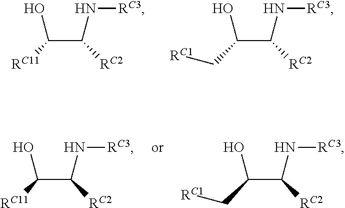

or a salt thereof, wherein R^{C11} is -L^{C1}-R^{C1}, L^{C1} is optionally substituted —CH₂—, R^{C1} is R, —Si(R)₃, —SO₂R or an electron-withdrawing group, and R^{C2} and R^{C3} are taken together with their intervening atoms to form an optionally substituted 3-10 membered saturated ring having, in addition to the nitrogen atom, 0-2 heteroatoms. In some embodiments, a useful chiral auxiliary has the structure of

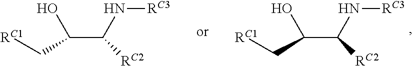

wherein R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms. is a formed ring is an optionally substituted 5-membered ring. In some embodiments, a useful chiral auxiliary has the structure of

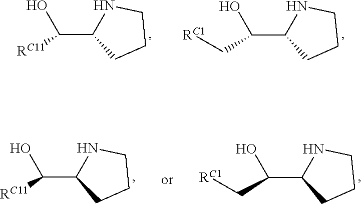

or a salt thereof. In some embodiments, a useful chiral auxiliary has the structure of

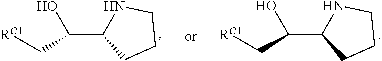

In some embodiments, a useful chiral auxiliary is a DPSE chiral auxiliary. In some embodiments, purity or stereochemical purity of a chiral auxiliary is at least 85%, 90%, 95%, 96%, 97%, 98%, or 99%. In some embodiments, it is at least 85%. In some embodiments, it is at least 90%. In some embodiments, it is at least 95%. In some embodiments, it is at least 96%. In some embodiments, it is at least 97%. In some embodiments, it is at least 98%. In some embodiments, it is at least 99%.

In some embodiments, L^{C1} is —CH₂—. In some embodiments, L^{C1} is substituted —CH₂—. In some embodiments, L^{C1} is mono-substituted —CH₂-.

In some embodiments, R^{C1} is R. In some embodiments, R^{C1} is optionally substituted phenyl. In some embodiments, R^{C1} is —SiR₃. In some embodiments, R^{C1} is -SiPh₂Me. In some embodiments, R^{C1} is —SO₂R. In some embodiments, R is not hydrogen. In some embodiments, R is optionally substituted phenyl. In some embodiments, R is phenyl. In some embodiments, R is optionally substituted C₁₋₆ aliphatic. In some embodiments, R is C₁₋₆ alkyl. In some embodiments, R is methyl. In some embodiments, R is t-butyl.

In some embodiments, R^{C1} is an electron-withdrawing group, such as —C(O)R, —OP(O)(OR)₂, —OP(O)(R)₂, —P(O)(R)₂, —S(O)R, —S(O)₂R, etc. In some embodiments, chiral auxiliaries comprising electron-withdrawing group R^{C1} groups are particularly useful for preparing chirally controlled non-negatively charged internucleotidic linkages and/or chirally controlled internucleotidic linkages bonded to natural RNA sugar.

In some embodiments, R^{C2} and R^{C3} are taken together with their intervening atoms to form an optionally substituted 3-10 (e.g., 3, 4, 5, 6, 7, 8, 9, or 10) membered saturated ring having no heteroatoms in addition to the nitrogen atom. In some embodiments, R^{C2} and R^{C3} are taken together with their intervening atoms to form an optionally substituted 5-membered saturated ring having no heteroatoms in addition to the nitrogen atom.

In some embodiments, a compound has the structure of H—X^{C}-C(R^{C5})₂-C(R^{C6})₂—SH or a salt thereof, wherein X^{C} is O or S, and each of R^{C5} and R^{C6} is independently R as described herein. In some embodiments, such a compound is useful for preparing a monomer. In some embodiments, such a compound is useful as a chiral auxiliary. In some embodiments, such a compound is particularly useful for preparing monomer which when utilized in oligonucleotide synthesis form bonds between their nitrogen atoms with linkage phosphorus (e.g., monomers comprising sm01, sm18, etc.). In some embodiments, X^{C} is O. In some embodiments, X^{C} is S. In some embodiments, one R^{C5} is —H. In some embodiments, one R^{C6} is —H. In some embodiments, a compound has the structure of H—X^{C}—CHR^{C5}—CHR^{C6}-SH or a salt thereof. In some embodiments, R^{C5} s is optionally substituted C₁₋₆ aliphatic. In some embodiments, R^{C5} is optionally substituted C₁₋₆ alkyl. In some embodiments, R^{C5} is methyl. In some embodiments, R^{C6} is optionally substituted C₁₋₆ aliphatic. In some embodiments, R^{C6} is optionally substituted C₁₋₆ alkyl. In some embodiments, R^{C6} is methyl. In some embodiments, a compound is HOCH(CH₃)CH(CH₃)SH. In some embodiments, a compound is HSCH(CH₃)CH(CH₃)SH. In some embodiments, one RCs is not hydrogen. In some embodiments, one R^{C6} is not hydrogen. In some embodiments, one R^{C5} and one R^{C6} are taken together with their intervening atoms to form an optionally substituted 3-20 (e.g., 3-15, 3-10, 5-10, 3, 4, 5, 6, 7, 8, 9, 10, 11, 12, 13, 14, 15, 16, 17, 18, 19, 20) membered monocyclic, bicyclic or polycyclic ring having 0-5 heteroatoms. In some embodiments, a formed ring is monocyclic. In some embodiments, one R^{C5} and one R^{C6} are taken together with their intervening atoms to form an optionally substituted 4-8, 4-7, 5-8, 5-7, 4, 5, 6, 7, or 8-membered monocyclic ring. In some embodiments, a formed ring is a saturated cycloalkyl ring. In some embodiments, a formed ring is a cyclohexyl ring. In some embodiments, a formed ring is bicyclic. In some embodiments, a formed ring contain no heteroatom ring atoms. In some embodiments, each monocyclic ring unit is independently 3-10 membered, and/or is independently saturated, partially unsaturated or aromatic and has 0-5 heteroatoms. In some embodiments, a compound is

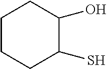

or a salt thereof, wherein the cyclohexyl ring is optionally substituted. In some embodiments, a compound is

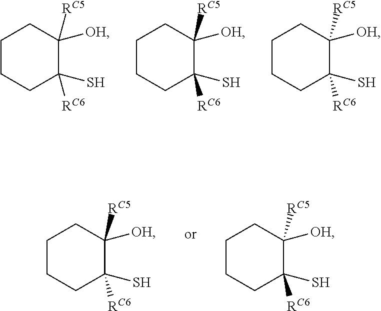

or a salt thereof, wherein the cyclohexyl ring is optionally substituted. In some embodiments, a substituent is C₁₋₆ aliphatic, e.g., —C(CH₃)=CH₂. For example, in some embodiments, a compound is

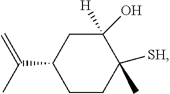

In some embodiments, a compound is

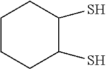

or a salt thereof, wherein the cyclohexyl ring is optionally substituted.

In some embodiments, methods for preparing oligonucleotides and/or compositions comprise using a chiral auxiliary described herein, e.g., for constructing one or more chirally controlled internucleotidic linkages. In some embodiments, one or more chirally controlled internucleotidic linkages are independently constructed using a DPSE chiral auxiliary. In some embodiments, each chirally controlled phosphorothioate internucleotidic linkage is independently constructed using a DPSE chiral auxiliary. In some embodiments, one or more chirally controlled internucleotidic linkages are independently constructed using

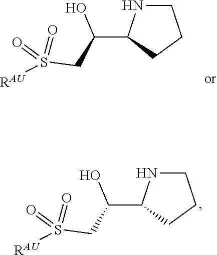

or a salt thereof, wherein R^{AU} is as described herein. In some embodiments, each chirally controlled non-negatively charged internucleotidic linkage (e.g., n001) is independently constructed using

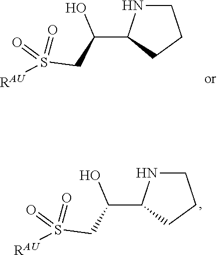

or a salt thereof. In some embodiments, each chirally controlled internucleotidic linkage is independently constructed using

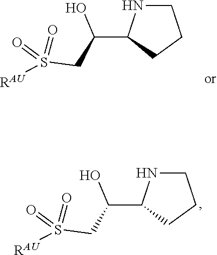

or a salt thereof. In some embodiments, R^{AU} is optionally substituted C₁₋₂₀, C₁₋₁₀, C₁₋₆, C₁₋₅, or C₁₋₄ aliphatic. In some embodiments, R^{AU} is optionally substituted C₁₋₂₀, C₁₋₁₀, C₁₋₆, C₁₋₅, or C₁₋₄ alkyl. In some embodiments, R^{AU} is optionally substituted aryl. In some embodiments, R^{AU} is phenyl. In some embodiments, one or more chirally controlled internucleotidic linkages are constructed using a PSM chiral auxiliary. In some embodiments, each chirally controlled non-negatively charged internucleotidic linkage (e.g., n001) is independently constructed using a PSM chiral auxiliary. In some embodiments, each chirally controlled internucleotidic linkages is independently constructed using a PSM chiral auxiliary. As appreciated by those skilled in the art, a chiral auxiliary is often utilized in a phosphoramidite

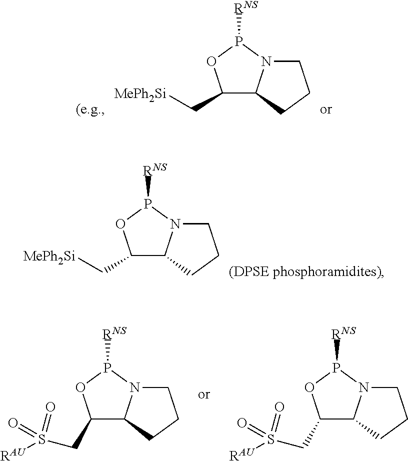

(wherein R^{AU} is independently as described herein; when R^{AU} is -Ph, PSM phosphoramidites), wherein R^{NS} is an optionally substituted/protected nucleoside (e.g., optionally protected for oligonucleotide synthesis), or a salt thereof, etc.) for oligonucleotide preparation. In some embodiments, a phosphoramidite is a compound having the structure of

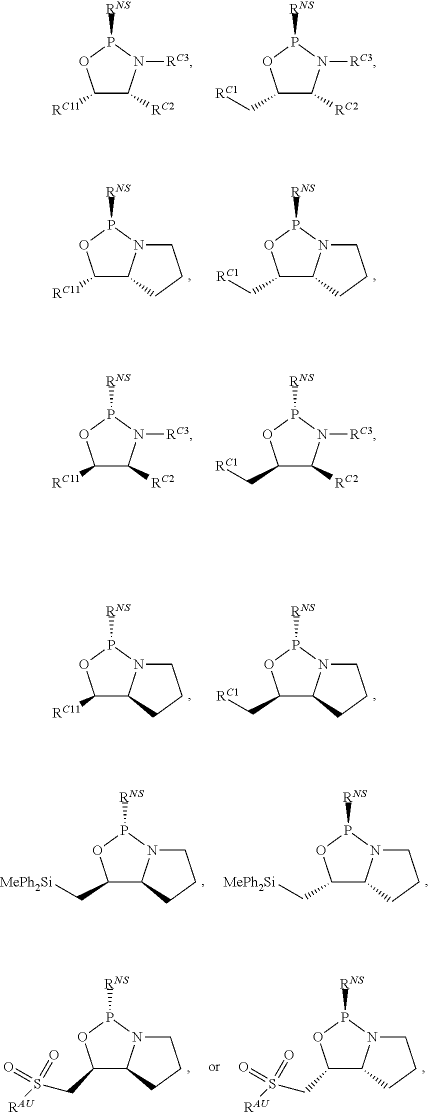

or salt thereof, wherein each variable is independently as described herein. In some embodiments. R^{AU} is optionally substituted phenyl. In some embodiments, R^{AU} is phenyl. In some embodiments, R^{NS} is an optionally substituted or protected nucleoside comprising hypoxanthine. In some embodiments, R^{NS} comprises optionally substituted or protected hypoxanthine. In some embodiments, R^{NS} is optionally substituted or protected inosine. In some embodiments, R^{NS} is optionally substituted or protected deoxyinosine. In some embodiments, R^{NS} is optionally substituted or protected 2′-F inosine (2′-OH replaced with 2′-F). In some embodiments, R^{N}S is optionally substituted or protected 2′-OR modified inosine (2′-OH replaced with a 2′-OR modification as described herein (e.g., 2′-OMe, 2′-MOE, etc.)). In some embodiments, hypoxanthine is O⁶ protected. In some embodiments, hypoxanthine is O⁶ protected with -L-Si(R)₃, wherein L is optionally substituted —CH₂—CH₂—, and each R is independently as described herein and not —H. In some embodiments, each R is independently an optionally substituted group selected from C₁₋₆ aliphatic and phenyl. In some embodiments, each R is independently optionally substituted C₁₋₆ alkyl. In some embodiments, -L-Si(R)₃ is —CH₂CH₂Si(Me)₃. In some embodiments, compounds comprising O⁶ protected hypoxanthine (e.g., with —CH₂CH₂Si(Me)₃) have higher solubility than corresponding O⁶ unprotected compounds and may provide various benefits and advantages when utilized for oligonucleotide synthesis in accordance with the present disclosure. In some embodiments, in a compound having the structure of

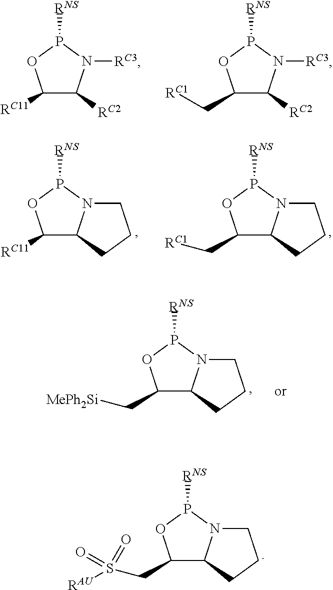

or salt thereof, R^{NS} comprises an O⁶ protected hypoxanthine (e.g., with —CH₂CH₂Si(Me)₃). In some embodiments, R^{NS} is O⁶-protected inosine. In some embodiments, RNS is O⁶-protected deoxyinosine. In some embodiments, RNS is O⁶-protected 2′-F inosine. In some embodiments, RNS is O⁶-protected 2′-OR modified inosine whose 2′-OR modification is as described herein (e.g., 2′-OMe, 2′-MOE, etc.). Among other things, the present disclosure encompasses the recognition that such a compound has sufficient solubility for oligonucleotide synthesis and can be utilized in oligonucleotide synthesis while a corresponding compound without O⁶ protection may not have sufficient solubility for efficient oligonucleotide synthesis. In some embodiments, a phosphoramidite is (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((methyldiphenylsilyl)methyl)tetrahydro-1H,3H-pyrrolo[1,2-c][1,3,2]oxazaphosphole. In some embodiments, a phosphoramidite is (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((phenylsulfonyl)methyl)tetrahydro-1H,3H-pyrrolo[1,2-c][1,3,2]oxazaphosphole. In some embodiments, in a compound having the structure of

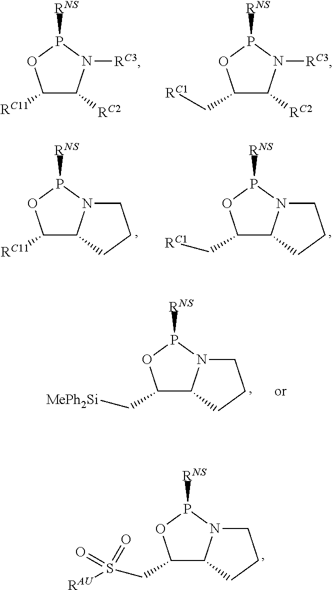

or salt thereof, R^{NS} comprises an O⁶ unprotected hypoxanthine. In some embodiments, R^{NS} is optionally substituted or protected inosine wherein the hypoxanthine is unprotected. In some embodiments, R^{NS} is optionally substituted or protected deoxyinosine wherein the hypoxanthine is unprotected. In some embodiments, R^{NS} is optionally substituted or protected 2′-F inosine wherein the hypoxanthine is unprotected. In some embodiments, R^{N}S is optionally substituted or protected 2′-OR modified inosine wherein the hypoxanthine is unprotected and whose 2′-OR modification is as described herein (e.g., 2′-OMe, 2′-MOE, etc.). Among other things, the present disclosure encompasses the recognition that such a compound has sufficient solubility for oligonucleotide synthesis and can be utilized in oligonucleotide synthesis without O⁶ protection.

In some embodiments, a method comprises providing a DPSE and/or a PSM phosphoramidite or a salt thereof. In some embodiments, a provided method comprises contacting a DPSE and/or a PSM phosphoramidite or a salt thereof with —OH (e.g., 5′—OH of a nucleoside or an oligonucleotide chain). As those skilled in the art appreciate, contacting can be performed under various suitable conditions so that a phosphorus linkage is formed. In some embodiments, preparation of each chirally controlled internucleotidic linkage independently comprises contacting a DPSE or PSM phosphoramidite or a salt thereof with —OH (e.g., 5′—OH of a nucleoside or an oligonucleotide chain). In some embodiments, preparation of each chirally controlled phosphorothioate internucleotidic linkage independently comprises contacting a DPSE phosphoramidite or a salt thereof with —OH (e.g., 5′—OH of a nucleoside or an oligonucleotide chain). In some embodiments, preparation of each chirally controlled non-negatively charged internucleotidic linkage (e.g., n001) independently comprises contacting a PSM phosphoramidite or a salt thereof with —OH (e.g., 5′—OH of a nucleoside or an oligonucleotide chain). In some embodiments, preparation of each chirally controlled internucleotidic linkage independently comprises contacting a PSM phosphoramidite or a salt thereof with —OH (e.g., 5′—OH of a nucleoside or an oligonucleotide chain). In some embodiments, contacting forms a P(III) linkage comprising a phosphorus atom bonded to two sugars and a chiral auxiliary moiety (e.g.,

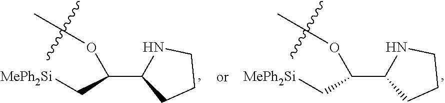

or a salt form thereof (e.g., from DPSE phosphoramidites or salts thereof),

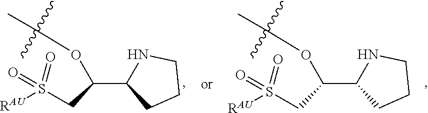

or a salt form thereof (wherein R^{AU} is independently as described herein; when R^{AU} is -Ph, e.g., from PSM phosphoramidites or salts thereof). etc.). In some embodiments, an oligonucleotide comprises a P(III) linkage comprising a chiral auxiliary moiety, e.g., from a DPSE or PSM phosphoramidite. In some embodiments, a P(III) linkage comprising a chiral auxiliary moiety is chirally controlled. In some embodiments, a chiral auxiliary moiety may be protected, e.g., before converting a P(III) linkage to a P(V) linkage (e.g., before sulfurization, reacting with azide, etc.). In some embodiments, a protected chiral auxiliary has the structure of

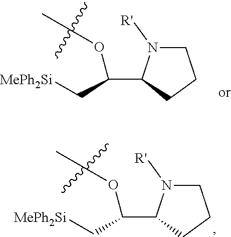

or a salt form thereof (e.g., wherein R′ is independently as described herein; e.g., from DPSE phosphoramidites or salts thereof), or

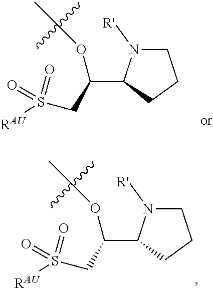

or a salt form thereof (wherein each R′ and R^{AU} is independently as described herein; when R^{AU} is -Ph, e.g., from PSM phosphoramidites or salts thereof), wherein each R′ is independently as described herein. In some embodiments, R′ is —C(O)R, wherein R is as described herein. In some embodiments, R is —CH₃. In some embodiments, an oligonucleotide comprises a protected chiral auxiliary. In some embodiments, each chirally controlled internucleotidic linkage in an oligonucleotide independently comprises

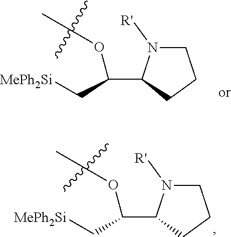

or a salt form thereof, or

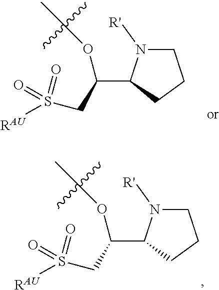

or a salt form thereof In some embodiments, each chirally controlled internucleotidic linkage in an oligonucleotide independently comprises

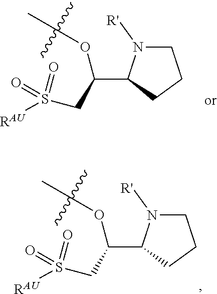

or a salt form thereof In some embodiments, R′ is —C(O)R. In some embodiments, R′ is —C(O)CH₃. In some embodiments, R^{AU} is Ph. In some embodiments, an oligonucleotide comprises one or more

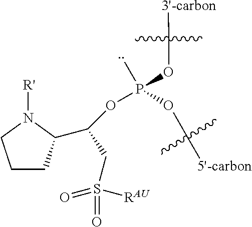

or a salt form thereof (PIII-1), wherein each variable independently as described herein. In some embodiments, an oligonucleotide comprises one or more

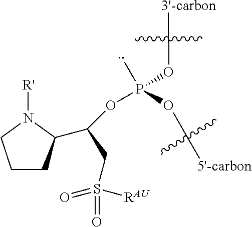

or a salt form thereof (PIII-2), wherein each variable independently as described herein. In some embodiments, an oligonucleotide comprises one or more

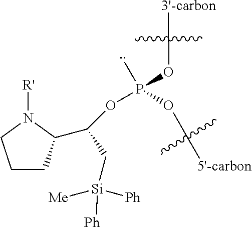

or a salt form thereof (PIII-5), wherein each variable independently as described herein. In some embodiments, an oligonucleotide comprises one or more

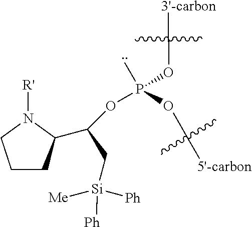

or a salt form thereof (PIII-6), wherein each variable independently as described herein. In some embodiments, a 5′-end internucleotidic linkage is PIII-1, PIII-2, PIII-5, or PIII-6. In some embodiments, a 5′-end internucleotidic linkage is PIII-1 or PIII-2. In some embodiments, R′ is —H. In some embodiments, R′ is —C(O)R. In some embodiments, R′ is —C(O)CH₃. In some embodiments, R^{AU} is -Ph. In some embodiments, a P(III) linkage is converted into a P(V) linkage. In some embodiments, a P(V) linkage comprises a phosphorus atom bonded to two sugars, a chiral auxiliary moiety

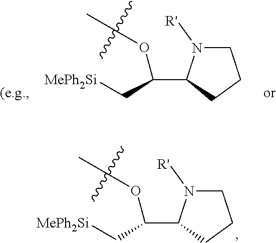

or a salt form thereof (wherein R‘ is as described herein; e.g., from DPSE phosphoramidites or salts thereof),

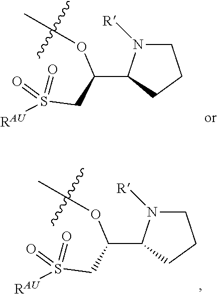

or a salt form thereof (wherein each of R′ and R^{AU} is independently as described herein; when R^{AU} is -Ph, e.g., from PSM phosphoramidites or salts thereof), etc.), and S or

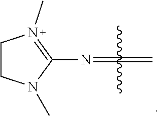

In some embodiments, a P(V) linkage comprises a phosphorus atom bonded to two sugars,

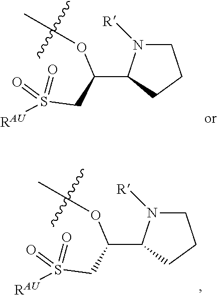

or a Salt form thereof (wherein each R′ and R^{AU} is independently as described herein; when R^{AU} is -Ph, e.g., from PSM phosphoramidites or salts thereof), etc.), and S or

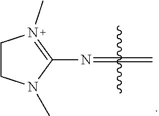

In some embodiments, a P(V) linkage comprises a phosphorus atom bonded to two sugars,

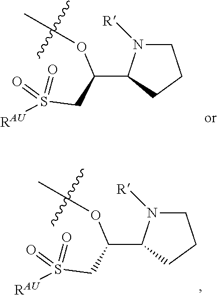

or a salt form thereof (wherein each R′ and R^{AU} is independently as described herein; when R^{AU} is -Ph, e.g., from PSM phosphoramidites or salts thereof), etc.), and S. In some embodiments, a P(V) linkage comprises a phosphorus atom bonded to two sugars,

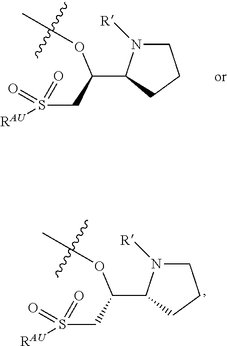

or a salt form thereof (wherein each R′ and R^{AU} is independently as described herein; when R^{AU} is -Ph, e.g., from PSM phosphoramidites or salts thereof), etc.), and

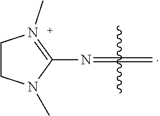

Those skilled in the art will appreciate that

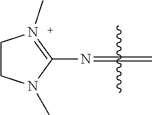

can exist with a counterion, e.g., in some embodiments, PF₆⁻. In some embodiments, an oligonucleotide comprises one or more

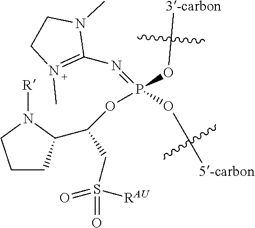

or a salt form thereof (PV-1), wherein each variable independently as described herein. In some embodiments, an oligonucleotide comprises one or more

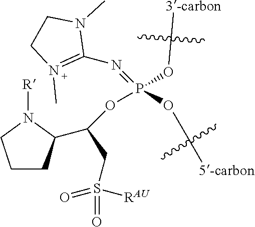

or a salt form thereof (PV-2), wherein each variable independently as described herein. In some embodiments, an oligonucleotide comprises one or more

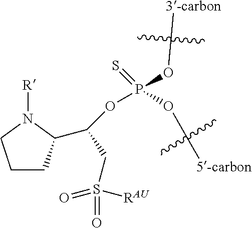

or a salt form thereof (PV-3), wherein each variable independently as described herein. In some embodiments, an oligonucleotide comprises one or more

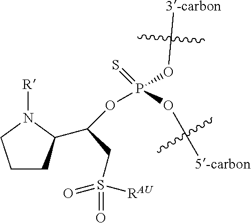

or a salt form thereof (PV-4), wherein each variable independently as described herein. In some embodiments, an oligonucleotide comprises one or more

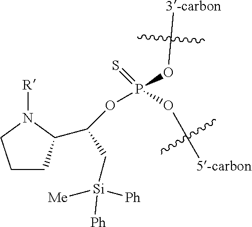

or a salt form thereof (PV-5), wherein each variable independently as described herein. In some embodiments, an oligonucleotide comprises one or more

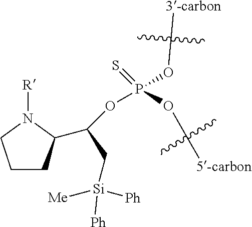

or a salt form thereof (PV-6), wherein each variable independently as described herein. In some embodiments, each chiral internucleotidic linkage, or each chirally controlled internucleotidic linkage, of an oligonucleotide is independently selected from PIII-1, PIII-2, PIII-5, PIII-6, PV-1, PV-2, PV-3, PV-4, PV-5, and PV-6. In some embodiments, each chiral internucleotidic linkage, or each chirally controlled internucleotidic linkage, of an oligonucleotide is independently selected from PIII-1, PIII-2, PV-1, PV-2, PV-3, and PV-4. In some embodiments, a linkage of PIII-1, PIII-2, PIII-5, or PIII-6 is typically the 5′-end internucleotidic linkage. In some embodiments, each chiral internucleotidic linkage, or each chirally controlled internucleotidic linkage, of an oligonucleotide is independently selected from PV-1, PV-2. PV-3, PV-4, PV-5, and PV-6. In some embodiments, each chiral internucleotidic linkage, or each chirally controlled internucleotidic linkage, of an oligonucleotide is independently selected from PV-1, PV-2, PV-3, or PV-4. In some embodiments, a provided oligonucleotide is an oligonucleotide as described herein, e.g., of Table 1, wherein each *S is independently replaced with PV-3 or PV-5, each *R is independently replaced with PV-4 or PV-6, each n001R is independently replaced with PV-1, and each n001S is independently replaced with PV-2. In some embodiments, a provided oligonucleotide is an oligonucleotide as described herein, e.g., of Table 1, wherein each *S is independently replaced with PV-3, each *R is independently replaced with PV-4, each n001R is independently replaced with PV-1, and each n001S is independently replaced with PV-2. In some embodiments, each natural phosphate linkage is independently replaced with a precursor, e.g.,

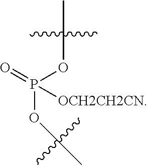

In some embodiments, R′ is —H. In some embodiments, R′ is —C(O)R. In some embodiments, R′ is —C(O)CH₃. In some embodiments, R^{AU} is -Ph. In some embodiments, a method comprises removal of one or more chiral auxiliary moieties so that phosphorothioate and/or non-negatively charged internucleotidic linkages (e.g., n001) are formed (e.g., from V-1, PV-2, PV-3, PV-4, PV-5, PV-6. etc.). In some embodiments, removal of a chiral auxiliary (e.g., PSM) comprises contacting an oligonucleotide with a base (e.g., N(R)₃ such as DEA) under anhydrous conditions.

In some embodiments, as appreciated by those skilled in the art, for preparation of a chirally controlled internucleotidic linkage, a monomer or a phosphoramidite (e.g., a DPSE or PSM phosphoramidite) is typically utilized in a chirally enriched or pure form (e.g., of a purity as described herein (e.g., about or at least about 90%, 91%, 92%, 93%, 94%, 95%, 96%, 97%, 98%, or 99%, or about 100%)).

In some embodiments, the present disclosure provides useful reagents for preparation of oligonucleotides and compositions thereof. In some embodiments, monomers and phosphoramidites comprise nucleosides, nucleobases and sugars as described herein. In some embodiments, nucleobases and sugars are properly protected for oligonucleotide synthesis as those skilled in the art will appreciate. In some embodiments, a phosphoramidite has the structure of R^{NS}P(OR)N(R)₂, wherein R^{NS} is a optionally protected nucleoside moiety. In some embodiments, a phosphoramidite has the structure of R^{NS}-P(OCH₂CH₂CN)N(i-Pr)₂. In some embodiments, a monomer comprises a nucleobase which is or comprises Ring BA, wherein Ring BA has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a phosphoramidite comprises a nucleobase which is or comprises Ring BA, wherein Ring BA has the structure of BA-1, BA-1-a, BA-1-b, BA-1-c, BA-1-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a phosphoramidite comprises a chiral auxiliary moiety, wherein the phosphorus is bonded to an oxygen and a nitrogen atom of the chiral auxiliary moiety. In some embodiments, a phosphoramidite has the structure of

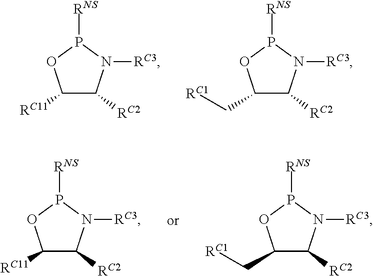

or a salt thereof, wherein R^{NS} is a protected nucleoside moiety (e.g., 5′—OH and/or nucleobases suitably protected for oligonucleotide synthesis), and each other variable is independently as described herein. In some embodiments, a phosphoramidite has the structure of

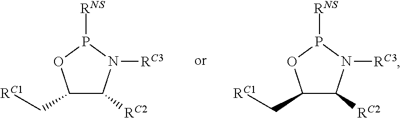

wherein R^{NS} is a protected nucleoside moiety (e.g., 5′—OH and/or nucleobases suitably protected for oligonucleotide synthesis), R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage. In some embodiments, 5′—OH of R^{NS} is protected. In some embodiments, 5′—OH of R^{NS} is protected as —ODMTr. In some embodiments, R^{NS} is bonded to phosphorus through its 3′—O—. In some embodiments, a formed ring by R^{C2} and R^{C3} is an optionally substituted 5-membered ring. In some embodiments, a phosphoramidite has the structure of

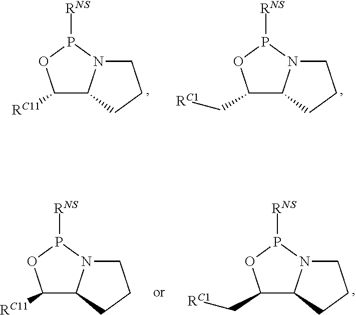

or a salt thereof. In some embodiments, a phosphoramidite has the structure of

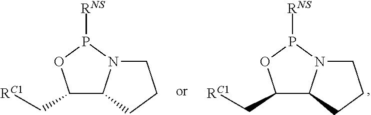

In some embodiments, as described herein R^{NS} comprises a modified nucleobase (e.g., b001A, b002A, b003A, b008U, b001C, etc.) which is optionally protected for oligonucleotide synthesis. In some embodiments, a monomer has the structure of

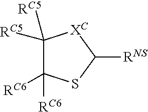

or a salt thereof, wherein R^{NS} is an optionally substituted/protected nucleoside (e.g., optionally protected for oligonucleotide synthesis) as described herein, and each other variable is independently as described herein. In some embodiments, -X^{C}-C(R^{C5})₂-C(R^{C6})₂-S- is of such a structure that H—X^{C}-C(R^{C5})₂-C(R^{C6})₂—SH is a compound as described herein, e.g., HOCH(CH₃)CH(CH₃)SH, HSCH(CH₃)CH(CH₃)SH,

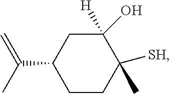

etc. In some embodiments, 5′—OH of R^{NS} is protected. In some embodiments, 5′—OH of R^{NS} is protected as —ODMTr.
In some embodiments, R^{NS} is an optionally substituted or protected nucleoside selected from

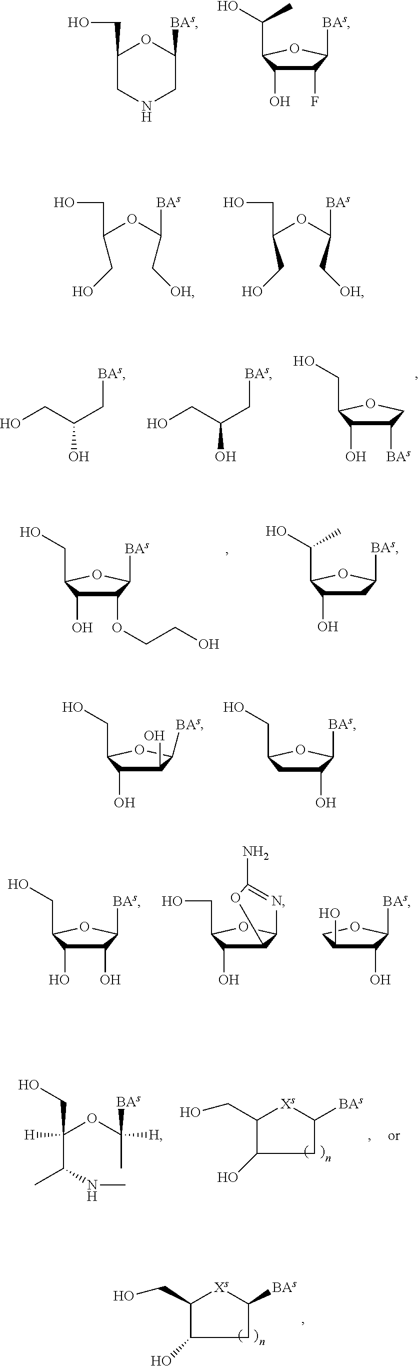

or a salt thereof, wherein BA^{s} is as described herein and each other variable is independently as described herein. In

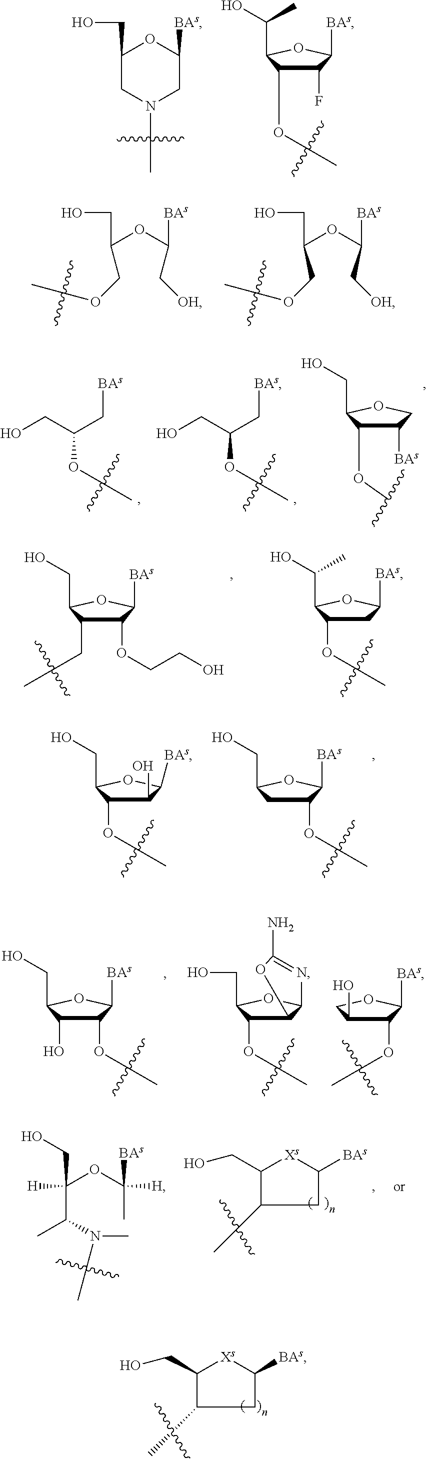

or a salt thereof, wherein BA^{s} is as described herein. In some embodiments, each —OH is optionally and independently substituted or protected. In some embodiments, BA^{s} is optionally substituted or protected nucleobase, and each —OH of the nucleoside is independently protected, wherein at least one —OH is protected as DMTrO—. In some embodiments, —OH for coupling, e.g., with another monomer or phosphoramidite, is protected as DMTrO—. In some embodiments, an —OH group for coupling, e.g., with another monomer or phosphoramidite, is protected different from an —OH group that is not for coupling. In some embodiments, a non-coupling —OH is protected such that the protection remains when DMTrO- is deprotected. In some embodiments, a non-coupling —OH is protected such that the protection remains during oligonucleotide synthesis cycles. In some embodiments, BAS is an optionally protected nucleobase selected from A, T, C, G, U, and tautomers thereof. In some embodiments, BA^{s} is an optionally protected nucleobase selected from Table BA-1 and tautomers thereof. In some embodiments, BA^{s} is an optionally protected nucleobase selected for b004A, b005A, b006A, b007A, b014U, and b015U, and tautomers thereof. In some embodiments, X^{s} is —O—. In some embodiments, X^{s} is -S-. In some embodiments, X^{s} is -Se-. In some embodiments, X^{s} is optionally substituted —CH₂—. In some embodiments, X^{s} is —CH₂—. In some embodiments, n is 0. In some embodiments, n is 1. In some embodiments, n is 2. In some embodiments, n is 3. In some embodiments, R^{NS} comprises an optionally substituted or protected nucleobase as described herein (e.g., selected from Table BA-1) or a tautomer thereof and a sugar as described herein.

In some embodiments, purity or stereochemical purity of a monomer or a phosphoramidite is at least 85%, 90%, 95%, 96%, 97%, 98%, or 99%. In some embodiments, it is at least 85%. In some embodiments, it is at least 90%. In some embodiments, it is at least 95%.

In some embodiments, the present disclosure provides a method for preparing an oligonucleotide or composition, comprising coupling a free —OH, e.g., a free 5′—OH, of an oligonucleotide or a nucleoside with a monomer as described herein. In some embodiments, the present disclosure provides a method for preparing an oligonucleotide or composition, comprising coupling a free —OH, e.g., a free 5′—OH, of an oligonucleotide or a nucleoside with a phosphoramidite as described herein.

In some embodiments, the present disclosure provides an oligonucleotide, wherein the oligonucleotide comprises one or more modified internucleotidic linkages each independently having the structure of -O⁵-P^{L}(W)(R^{CA})—O³—, wherein:
P^{L} is P, or P(═W);
W is O, S, or W^{N};
W^{N} is =N-C(—N(R¹)₂=N⁺(R¹)₂Q⁻;
Q⁻ is an anion;
R^{CA} is or comprises an optionally capped chiral auxiliary moiety,
O⁵ is an oxygen bonded to a 5′-carbon of a sugar, and
O is an oxygen bonded to a 3′-carbon of a sugar.

In some embodiments, a modified internucleotidic linkage is optionally chirally controlled. In some embodiments, a modified internucleotidic linkage is optionally chirally controlled.

In some embodiments, a provided methods comprising removing R^{CA} from such a modified internucleotidic linkages. In some embodiments, after removal, bonding to R^{CA} is replaced with —OH. In some embodiments, after removal, bonding to R^{CA} is replaced with ═O, and bonding to W^{N} is replaced with -N═C(N(R¹)₂)₂.

In some embodiments, P^{L} is P═S, and when R^{CA} is removed, such an internucleotidic linkage is converted into a phosphorothioate internucleotidic linkage.

In some embodiments, P^{L} is P=W^{N}, and when R^{CA} is removed, such an internucleotidic linkage is converted into an internucleotidic linkage having the structure of

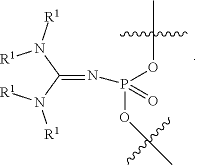

In some embodiments, an internucleotidic linkage having the structure of

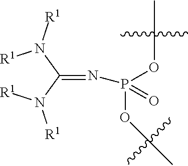

has the structure of

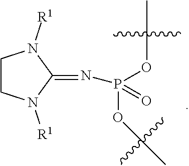

In some embodiments, an internucleotidic linkage having the structure of

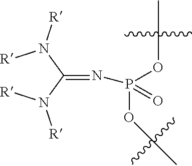

has the structure of

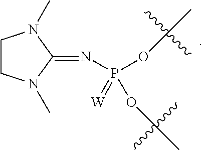

In some embodiments, P^{L} is P (e.g., in newly formed internucleotidic linkage from coupling of a phosphoramidite with a 5′—OH). In some embodiments, W is O or S. In some embodiments, W is S (e.g., after sulfurization). In some embodiments, W is O (e.g., after oxidation). In some embodiments, certain non-negatively charged internucleotidic linkages or neutral internucleotidic linkages may be prepared by reacting a P(III) phosphite triester internucleotidic linkage with azido imidazolinium salts (e.g., compounds comprising

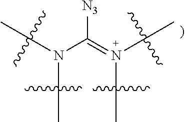

under suitable conditions. In some embodiments, an azido imidazolinium salt is a salt of PF₆⁻. In some embodiments, an azido imidazolinium salt is a slat of

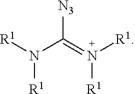

In some embodiments, an azido imidazolinium salt is 2-azido-1,3-dimethylimidazolinium hexafluorophosphate.

As appreciated by those skilled in the art, Q⁻ can be various suitable anion present in a system (e.g., in oligonucleotide synthesis), and may vary during oligonucleotide preparation processes depending on cycles, process stages, reagents, solvents, etc. In some embodiments, Q⁻ is PF₆⁻.

In some embodiments, R^{CA} is

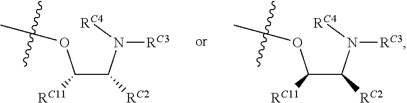

wherein R^{C4} is —H or —C(O)R′, and each other variable is independently as described herein. In some embodiments, R^{CA} is

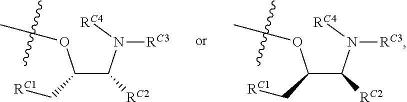

wherein R^{C1} is R, —Si(R)₃ or —SO₂R, R^{C2} and R^{C3} are taken together with their intervening atoms to form an optionally substituted 3-7 membered saturated ring having, in addition to the nitrogen atom, 0-2 heteroatoms, R^{C4} is —H or —C(O)R′. In some embodiments, R^{C4} is —H. In some embodiments, RC⁴ is —C(O)CH₃. In some embodiments, R^{C2} and R^{C3} are taken together to form an optionally substituted 5-membered ring.

In some embodiments, R^{C4} is —H (e.g., in n newly formed internucleotidic linkage from coupling of a phosphoramidite with a 5′—OH). In some embodiments, R^{C4} is —C(O)R (e.g., after capping of the amine). In some embodiments, R is methyl.

In some embodiments, each chirally controlled phosphorothioate internucleotidic linkage is independently converted from —O⁵—P^{L}(W)(R^{CA})-O³_

### Assessment Characterization of Providing Technologies

As appreciated by those skilled in the art, various technologies may be utilized to assess/characterize provided technologies in accordance with the present disclosure. Among other things, the present disclosure describes various in vivo and in vitro technologies suitable for assessing and characterizing provided technologies. Certain useful technologies are described in the Examples, WO 2021/071858, WO 2022/046667, WO 2022/099159, and/or PCT/US2023/018742. In some embodiments, provided technologies are assessed/characterized, e.g., in cells, with or without exogenous ADAR polypeptides; additionally or alternatively, in some embodiments, provided technologies are assessed/characterized, e.g., in animals, e.g., non-human primates and mice.

Among other things, the present disclosure encompasses the insights that various agents (e.g., oligonucleotides) and compositions thereof that can provide editing in various human systems, e.g., cells, may show no or much lower levels of editing in certain cells (e.g., mouse cells) and certain animals such as rodents (e.g., mice) that do not contain or express human ADAR, e.g., human ADAR1. Particularly, mice, a commonly used animal model, may be of limited uses for assessing various agents (e.g., oligonucleotides) for editing in humans, as various agents active in human cells provide no or very low levels of activity in mouse cells and animals not engineered to comprise or express a proper ADAR1 (e.g., human ADAR1) polypeptide or a characteristic portion thereof In some embodiments, the present disclosure provides engineered cells and non-human animals expressing human ADAR1 polypeptide or a characteristic portion thereof. In some embodiments, such cells and human are useful for assessing and characterizing provided technologies. In some embodiments, a human ADAR1 polypeptide or a characteristic portion thereof is or comprises human ADAR1 polypeptide or a characteristic portion thereof. In some embodiments, a human ADAR1 polypeptide or a characteristic portion thereof is or comprises human ADAR1 p110 polypeptide or a characteristic portion thereof In some embodiments, a human ADAR1 polypeptide or a characteristic portion thereof is or comprises human ADAR1 p150 polypeptide or a characteristic portion thereof. In some embodiments, a human ADAR1 polypeptide or a characteristic portion thereof is or comprises human ADAR1. In some embodiments, a human ADAR1 polypeptide or a characteristic portion thereof is or comprises a human ADAR1 p110 peptide. In some embodiments, a human ADAR1 polypeptide or a characteristic portion thereof is or comprises a human ADAR1 p150 peptide. In some embodiments, a human ADAR1 polypeptide or a characteristic portion thereof is or comprises one or more or all of the following domains of human ADAR1: Z-DNA binding domains, dsRNA binding domains, and deaminase domain. In some embodiments, a human ADAR1 polypeptide or a characteristic portion thereof is or comprises one or both of human ADAR1 Z-DNA binding domains; alternatively or additionally, in some embodiments, a human ADAR1 polypeptide or a characteristic portion thereof is or comprises one, two or all of human ADAR1 dsRNA binding domains; alternatively or additionally, a human ADAR1 polypeptide or a characteristic portion thereof is or comprises a human deaminase domain. In some embodiments, a human ADAR1 polypeptide or a characteristic portion thereof may be expressed together with a mouse ADAR1 polypeptide or a characteristic portion thereof, e.g., one or more human dsRNA binding domains may be engineered to be expressed together with a mouse deaminase domain to form a human-mouse hybrid ADAR1 polypeptide. In some embodiments, cells and/or non-human animals are engineered to comprise and/or express a polynucleotide encoding a human ADAR1 polypeptide or a characteristic portion thereof as described herein. In some embodiments, genomes of cells and/or non-human animals are engineered to comprise a polynucleotide encoding a human ADAR1 polypeptide or a characteristic portion thereof as described herein. In some embodiments, germline genomes of cells and/or non-human animals are engineered to comprise a polynucleotide encoding a human ADAR1 polypeptide or a characteristic portion thereof as described herein. In some embodiments, cells and non-human animals are engineered to comprise, e.g., in their genomes (in some embodiments, germline genomes), one or more G to A mutations each independently associated with a condition, disorder or disease (e.g., a mutation (e.g., c. 1024G>A) in SERPINA1 gene that leads to a glutamate to lysine substitution at amino acid position 342 (E342K) of an A1AT protein). Among other things, such cells and animals are useful for assessing/characterizing provided technologies, e.g., various oligonucleotides and compositions thereof, e.g., for their editing properties and/or activities, including for their uses against one or more conditions, disorders or diseases. In some embodiments, cells are rodent cells. In some embodiments, cells are mouse cells. In some embodiments, an animal is a rodent. In some embodiments, an animal is a mice.

Among other things, the present disclosure provides oligonucleotide designs comprising sugar modifications, base modifications, internucleotidic linkage modifications, linkage phosphorus stereochemistry, and/or patterns thereof, that can greatly improve one or more properties and/or activities of oligonucleotides compared to comparable oligonucleotides of similar or identical base sequences but of reference designs. For example, oligonucleotides of various provided designs and compositions thereof can provide high levels of editing in mice that do not express a human ADAR protein (e.g., mice only expressing mouse ADAR proteins), in some embodiments comparable to or no lower than in mice that are engineered to express a human ADAR protein, while comparable oligonucleotides of reference designs and compositions thereof provide low levels of editing in mice that do not express a human ADAR protein (e.g., mice only expressing mouse ADAR proteins), in some embodiments significantly lower than in mice that are engineered to express a human ADAR protein. In some embodiments, a reference design is a design reported in WO 2016/097212, WO 2017/220751, WO 2018/041973, WO 2018/134301A1, WO 2019/158475, WO 2019/219581, WO 2020/157008, WO 2020/165077. WO 2020/201406, WO 2020/252376 WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256. In some embodiments, a reference design is a design in WO 2021/071858, WO 2022/099159, and/or PCT/US2023/018742.

In some embodiments, the present disclosure provides technologies for assessing/characterizing for assessing cells and/or non-human animals. including those engineered to comprise or express an ADAR1 polypeptide or a characteristic portion thereof, or a polynucleotide encoding an ADAR1 polypeptide or a characteristic portion thereof, which ADAR1 polypeptide or a characteristic portion thereof and/or polynucleotide is not in and/or is not expressed in the cells and/or non-human animals prior to engineering. In some embodiments, a provided method comprises administering to a cell or a population thereof one or more oligonucleotides or compositions which one or more oligonucleotides or compositions can each independently edit an adenosine in a comparable human cell or a population thereof. In some embodiments, a provided method comprises administering to an animal or a population thereof one or more oligonucleotides or compositions which one or more oligonucleotides or compositions can each independently edit an adenosine in a human cell or a population thereof In some embodiments, editing levels in cells to be assessed/characterized, or in cells from animals, are compared to those observed in comparable human cells. In some embodiments, comparable human cells are of the same type as cells to be assessed/characterized or cells from animals. In some embodiments, cells are rodent cells. In some embodiments, cells are mouse cells. In some embodiments, an animal is a rodent. In some embodiments, an animal is a mice. In some embodiments, one or more oligonucleotides or compositions are administered separately to separate cells and/or animals. In some embodiments, one or more oligonucleotides or compositions may be administered to the same collection of cells and/or animals, optionally simultaneously. Various oligonucleotides and compositions that can edit various target adenosines are as described herein and can be utilized accordingly.

As appreciated by those skilled in the art, in some embodiments, provided technologies, e.g., oligonucleotides, compositions, etc., may be assessed in one or more models, e.g., cells, tissues, organs, animals, etc. In some embodiments, as appreciated by those skilled in the art, cells, tissues, organs, animals, etc. are or comprise cells of, associated with or comprising one or more characteristics (e.g., nucleotide sequences such as mutations) of conditions, disorders or diseases. For example, in some embodiments, cells, tissues, organs, animals, etc. comprise G to A mutations associated with conditions, disorders or diseases, e.g., 1024G>A (E342K) in human SERPINA1. In some embodiments, an animal is a NOD.Cg-Prkdcscid Il2rgtm1Wjl Tg(SERPINA1*E342K) #Slcw/SzJ mouse (e.g., see The Jackson Laboratory Stock No: 028842; NSG-PiZ, and also Borel F; Tang Q; Gernoux G; Greer C; Wang Z; Barzel A; Kay MA; Shultz LD; Greiner DL; Flotte TR; Brehm MA; Mueller C. 2017. Survival Advantage of Both Human Hepatocyte Xenografts and Genome-Edited Hepatocytes for Treatment of alpha-1 Antitrypsin Deficiency. Mol Ther 25(11):2477-2489PubMed: 29032169MGI: J:243726, and Li S; Ling C; Zhong L; Li M; Su Q; He R; Tang Q; Greiner DL; Shultz LD; Brehm MA; Flotte TR; Mueller C; Srivastava A; Gao G. 2015). Efficient and Targeted Transduction of Nonhuman Primate Liver With Systemically Delivered Optimized AAV3B Vectors. Mol Ther 23(12):1867-76PubMed: 26403887MGI: J:230567). In some embodiments, cells, tissues, organs, animals, etc. comprise one or more cancer cells. In some embodiments, non-human cells, tissues, organs, animals, etc. are engineered to comprise or express ADAR1 or a characteristic portion thereof, e.g., through incorporation of (optionally into its genome or germline genome) a polynucleotide whose sequence encodes an ADAR1 polypeptide or a characteristic portion thereof In some embodiments, an ADAR1 is a primate ADAR1. In some embodiments, an ADAR1 is a human ADAR1. In some embodiments, a human ADAR1 is human ADAR1 p110. In some embodiments, a human ADAR1 is human ADAR1 p150. As appreciated by those skilled in the art, various technologies are available in the art and can be utilized in accordance with the present disclosure to generated useful cells, tissues, organs, animals, etc. For example, for condition, disorder or disease animal models expressing human ADAR1 or a characteristic portion thereof, an animal model can be crossed with huADAR1 mice described herein to provide engineered animal models expressing human ADAR1 or a characteristic portion thereof. In some embodiments, mice comprising G to A mutations, e.g., a NOD.Cg-Prkdcscid Il2rgtm1Wjl Tg(SERPINA1*E342K)#Slcw/SzJ mouse (e.g., see The Jackson Laboratory Stock No: 028842; NSG-PiZ, and also Borel F; Tang Q; Gernoux G; Greer C; Wang Z; Barzel A; Kay MA; Shultz LD; Greiner DL; Flotte TR; Brehm MA; Mueller C. 2017. Survival Advantage of Both Human Hepatocyte Xenografts and Genome-Edited Hepatocytes for Treatment of alpha-1 Antitrypsin Deficiency. Mol Ther 25(11):2477-2489PubMed: 29032169MGI: J:243726, and Li S; Ling C; Zhong L; Li M; Su Q; He R; Tang Q; Greiner D L; Shultz L D; Brehm M A; Flotte T R; Mueller C; Srivastava A; Gao G. 2015) are crossed with huADAR1 mice described herein to provide mice comprising G to A mutations (e.g., 024G>A (E342K) in human SERPINA1) and expressing human ADAR1 or a characteristic portion thereof.

As appreciated by those skilled in the art, in some embodiments, animals can be heterozygous with respect to one or more or all sequences. In some embodiments, animals are homozygous with respect to one or more or all sequences. In some embodiments, animals are hemizygous with respect to one or more or all engineered sequences. In some embodiments, animals are homozygous with respect to one or more sequences, and heterozygous with respect to one or more sequences. In some embodiments, animals are heterozygous with respect to a polynucleotide whose sequence encodes an ADAR1 polypeptide or a characteristic portion thereof In some embodiments, animals are homozygous with respect to a polynucleotide whose sequence encodes an ADAR1 polypeptide or a characteristic portion thereof In some embodiments, certain animals are heterozygous with respect to one or more polynucleotide sequences associated with various condition, disorder or diseases, and are heterozygous with respect to a polynucleotide whose sequence encodes an ADAR1 polypeptide or a characteristic portion thereof. In some embodiments, certain animals are homozygous with respect to one or more polynucleotide sequences associated with various condition, disorder or diseases, and are heterozygous with respect to a polynucleotide whose sequence encodes an ADAR1 polypeptide or a characteristic portion thereof In some embodiments, certain animals are heterozygous with respect to one or more polynucleotide sequences associated with various condition, disorder or diseases, and are homozygous with respect to a polynucleotide whose sequence encodes an ADAR1 polypeptide or a characteristic portion thereof In some embodiments, certain animals are homozygous with respect to one or more polynucleotide sequences associated with various condition, disorder or diseases, and are homozygous with respect to a polynucleotide whose sequence encodes an ADAR1 polypeptide or a characteristic portion thereof. Cells or tissues may be similarly heterozygous, hemizygous and/or homozygous with respect to various sequences.

Certain useful technologies, e.g., cells, animals, etc., are described in WO 2022/046667 and may be utilized in accordance with the present disclosure.

In some embodiments, the present disclosure provides methods for assessing an agent, e.g., an oligonucleotide, or a composition thereof, comprising administering to an animal, cell or tissue described herein the agent or composition. In some embodiments, an agent or composition is assessed for preventing or treating a condition, disorder or disease. In some embodiments, animals, cells, tissues, e.g., as described in various embodiments herein, are animal models, or cells or tissues, for various conditions, disorders or diseases (e.g., comprising mutations associated with various conditions, disorders or diseases, and/or cells, tissues, organs, etc., associated with or of various conditions, disorders or diseases) that are engineered to comprise and/or express a polynucleotide whose sequence encodes an ADAR1 polypeptide or a characteristic portion thereof In some embodiments, animals may be provided by breeding (e.g., IVF, natural breeding, etc.) an animal that are model animals for various conditions, disorders or diseases but are not engineered to comprise and/or express a polynucleotide whose sequence encodes an ADAR1 polypeptide or a characteristic portion thereof with animals that are engineered to comprise and/or express a polynucleotide whose sequence encodes an ADAR1 polypeptide or a characteristic portion thereof. In some embodiments, cells or tissues may be provided by introducing into cells or tissues a polynucleotide whose sequence encodes an ADAR1 polypeptide or a characteristic portion thereof In some embodiments, the present disclosure provides a method for preventing or treating a condition, disorder or disease, comprising administering to a subject an effective amount of an agent or a compositions thereof, wherein the agent or composition is assessed in an animal provided herein (e.g., an animal engineered to comprise an ADAR1 polypeptide or a characteristic portion thereof, an animal engineered to comprise and/or express a polynucleotide whose sequence encodes an ADAR1 polypeptide or a characteristic portion thereof, a model animal for a condition, disorder or disease which is engineered to comprise an ADAR1 polypeptide or a characteristic portion thereof, a model animal for a condition, disorder or disease engineered to comprise and/or express a polynucleotide whose sequence encodes an ADAR1 polypeptide or a characteristic portion thereof). In some embodiments, the present disclosure provides a method for preventing or treating a condition, disorder or disease, comprising administering to a subject an effective amount of an agent or a compositions thereof, wherein the agent or composition is assessed in a cell or tissue provided herein. In some embodiments, an animal, cell or tissue comprises a SERPINA1 mutation (e.g., 1024 G>A (E342K)) and is engineered to comprise and/or express a polynucleotide whose sequence encodes an ADAR1 polypeptide or a characteristic portion thereof In some embodiments, an animal is a non-human animal. In some embodiments, cells are non-human animal cells. In some embodiments, tissues are non-human animal tissues. In some embodiments, a non-human animal is a rodent. In some embodiments, a non-human animal is a mouse. In some embodiments, a non-human animal is a rat. In some embodiments, a non-human animal is a non-human primate.

In some embodiments, the present disclosure provides methods comprising: 1) assessing an agent or a composition thereof, comprising contacting the agent or a composition thereof with a provided cell or tissue associated with or of a condition, disorder or disease, and 2) administering to a subject suffering from or susceptible to a condition, disorder or disease an effective amount of an agent or composition thereof. In some embodiments, the present disclosure provides methods comprising: 1) assessing an agent or a composition thereof, comprising administering the agent or a composition thereof to a provided animal which is an animal model of a condition, disorder or disease, and 2) administering to a subject suffering from or susceptible to a condition, disorder or disease an effective amount of an agent or composition thereof. In some embodiments, as described herein, a cell, tissue or animal is engineered to comprise an ADAR1 polypeptide or a characteristic portion thereof. In some embodiments, a cell, tissue or animal is engineered to comprise and/or express a polynucleotide whose sequence encodes an ADAR1 polypeptide or a characteristic portion thereof. In some embodiments, a cell, tissue or animal further comprises a nucleotide sequence (e.g., a mutation) associated with a condition, disorder or disease. In some embodiments, an animal is a rodent, e.g., a mouse, a rat, etc. In some embodiments, a cell or tissue is of a rodent, e.g., a mouse, a rat, etc. In some embodiments, a cell is a germline cell. In some embodiments, a fraction of and not all cells, e.g., cells of particular cell types or tissues or location, of a population of cells, a tissue or an animal comprise a nucleotide sequence (e.g., a mutation) associated with a condition, disorder or disease, and such fraction of cells are engineered to comprise an ADAR1 polypeptide or a characteristic portion thereof or engineered to comprise and/or express a polynucleotide whose sequence encodes an ADAR1 polypeptide or a characteristic portion thereof In some embodiments, a collection of liver cells comprise a SERPINA1 mutation, e.g., 1024 G>A (E342K) and a polynucleotide whose sequence encodes an ADAR1 polypeptide or a characteristic portion thereof. Those skilled in the art appreciate that various technologies are available for optionally controlled introduction and/or expression of a nucleotide sequence in various cells, tissues, or organs and can be utilized in accordance with the present disclosure. In some embodiments, as described herein, a cell, tissue or animal comprises a polynucleotide whose sequence encodes an ADAR1 polypeptide or a characteristic portion thereof in a genome, in some embodiments, in a germline genome. In some embodiments, as described herein, a cell, tissue or animal comprises a nucleotide sequence (e.g., a mutation) associated with a condition, disorder or disease in a genome, in some embodiments, in a germline genome.

As described herein, in some embodiments, a polynucleotide encodes human ADAR1 p110 or a characteristic portion thereof In some embodiments, a polynucleotide encodes human ADAR1 p110. In some embodiments, a polynucleotide encodes human ADAR1 p150 or a characteristic portion thereof. In some embodiments, a polynucleotide encodes human ADAR1 p150. In some embodiments, a cell, tissue or animal (e.g., a huADAR mouse or a cell or tissue therefrom) is engineered to comprise and/or express a polynucleotide whose sequence encodes a human ADAR1 p110 polypeptide or a characteristic portion thereof In some embodiments, a cell, tissue or animal (e.g., a huADAR mouse or a cell or tissue therefrom) is engineered to comprise and/or express a polynucleotide whose sequence encodes a human ADAR1 p110 polypeptide. In some embodiments, a cell, tissue or animal (e.g., a huADAR mouse or a cell or tissue therefrom) is engineered to comprise and/or express a polynucleotide whose sequence encodes a human ADAR1 p150 polypeptide or a characteristic portion thereof. In some embodiments, a cell, tissue or animal (e.g., a huADAR mouse or a cell or tissue therefrom) is engineered to comprise and/or express a polynucleotide whose sequence encodes a human ADAR1 p150 polypeptide. As described herein, in some embodiments, an animal is a rodent, e.g., a mouse or a rat.

In some embodiments, ADAR (e.g., human ADAR1) transgene is established on a zygote, e.g., SERPINA1 mouse zygote comprising a mutation (e.g., 1024 G>A (E342K) in human SERPINA1) or vice versa. In some embodiments, a zygote is homozygous. In some embodiments, a zygote is heterozygous.

### Uses and Applications

As appreciated by those skilled in the art, oligonucleotides are useful for multiple purposes. In some embodiments, provided technologies (e.g., oligonucleotides, compositions, methods, etc.) can be useful for modulating levels and/or activities of various nucleic acids (e.g., RNA) and/or products encoded thereby (e.g., proteins). In some embodiments, provided technologies can reduce levels and/or activities of undesired target nucleic acids (e.g., comprising undesired adenosine) and/or products thereof. In some embodiments, provided technologies can increase levels and/or activities of desired target nucleic acids (e.g., comprising I instead of undesired adenosine at one or more locations) and/or products thereof.

For example, in some embodiments, provided technologies can be utilized as single-stranded oligonucleotides for site-directed editing of target adenosine in target RNA sequences. In some embodiments, provided technologies are capable of modulating levels of expressions and activities. Among other things, the present disclosure provides improvement by provided technologies which can be improvement of various desired biological functions, including but not limited to treatment and/or prevention of various conditions, disorders or diseases (e.g., those associated with G to A mutation).

In some embodiments, provided technologies can modulate activities and/or functions of a target gene. In some embodiments, a target gene is a gene with respect to which expression and/or activity of one or more gene products (e.g., RNA and/or protein products) are intended to be altered. In many embodiments, target genes have target adenosine residues to be altered and can benefit from conversion of such residues to inosine residues. In some embodiments, when an oligonucleotide as described herein acts on a particular target gene, level and/or activity of one or more gene products of that gene can be altered when the oligonucleotide is present as compared with when it is absent.

In some embodiments, provided oligonucleotides and compositions are useful for treating various conditions, disorders, or diseases, by reducing levels and/or activities of target transcripts and/or products encoded thereby that are associated with the conditions, disorders, or diseases, and optionally providing transcripts and/or products encoded thereby that are less associated or not associated with the conditions, disorders or diseases (e.g., by conversion of target adenosine to inosine to correct G to A mutations, to alter splicing, etc.). In some embodiments, the present disclosure provides methods for preventing or treating a condition, disorder, or disease, comprising administering to a subject susceptible thereto or suffering therefrom an effective amount of a provided oligonucleotide or composition. In some embodiments, the present disclosure provides methods for preventing or treating a condition, disorder, or disease, comprising administering to a subject susceptible to or suffering from a condition, disorder or disease a provided single-stranded oligonucleotide for site-directed editing of a nucleotide (e.g. target adenosine) in a target RNA sequence, or a composition thereof. In some embodiments, a provided single-stranded oligonucleotide for site-directed editing of a nucleotide in a target RNA sequence is of a base sequence that partially or fully complementary to a portion of a transcript, which transcript is associated with a condition, disorder, or disease. In some embodiments, a base sequence is such that it preferentially binds to a transcript associated with a condition, disorder or disease over other transcripts that are not associated with said condition, disorder, or disease. In some embodiments, a condition, disorder, or disease is associated with a G to A mutation. In some embodiments, a condition, disorder, or disease is associated with a G to A mutation in SERPINA1 In some embodiments, a condition, disorder, or disease is associated with 1024 G>A (E342K) mutation in human SERPINA1. In some embodiments, a condition, disorder or disease is alpha-1 antitrypsin deficiency. In some embodiments, provided technologies increase levels, properties, and/or activities of desired products (e.g., properly folded wild-type A1AT protein in serum) and/or decreases levels, properties, and/or activities of undesired products (e.g., mutant (e.g., E342K) A1AT protein in serum), in absolute amounts (e.g., ng/mL in serum) and/or relatively (e.g., as % of total proteins or total AAT proteins). In some embodiments, the present disclosure provides a method for increasing levels and/or activities of an alpha-1 antitrypsin (A1AT) polypeptide in the serum or blood of a subject, comprising administering to the subject an effective amount of an oligonucleotide or composition. In some embodiments, an A1AT polypeptide provides one or more higher activities compared to a reference A1AT polypeptide. In some embodiments, an A1AT polypeptide is a wild-type A1AT polypeptide. In some embodiments, method increase the amount of the A1AT polypeptide in serum. In some embodiments, a method decrease the amount of a reference A1AT polypeptide in serum. In some embodiments, a method increase the ratio of the A1AT polypeptide over a reference A1AT polypeptide in serum or blood. In some embodiments, a reference A1AT polypeptide is mutated. In some embodiments, a reference A1AT polypeptide is not properly folded. In some embodiments, a reference A1AT polypeptide is an E342K A1AT polypeptide. In some embodiments, the present disclosure provides a method for decreasing levels and/or activities of a mutant alpha-1 antitrypsin (A1AT) polypeptide in the serum or blood of a subject, comprising administering to the subject an effective amount of an oligonucleotide or composition. In some embodiments, a subject is susceptible to or suffering from a condition, disorder or disease. In some embodiments, a condition, disorder or disease is alpha-1 antitrypsin deficiency. In some embodiments, a subject is a human. In some embodiments, a subject comprises a mutation in human SERPINA1. In some embodiments, a subject comprises 1024 G>A (E342K) mutation in human SERPNA1. In some embodiments, a subject is homozygous with respect to the mutation. In some embodiments, a subject is heterozygous with respect to a mutation.

In some embodiments, a condition, disorder or disease is not associated with a G to A mutation. In some embodiments, a condition, disorder or disease is associated with increased level and/or activity of a transcript and/or an encoded product thereby, and a provided technology can reduce level and/or activity of a transcript and/or an encoded product thereby, e.g., through introducing one or more A to I to a transcript. In some embodiments, a condition, disorder or disease is associated with decreased level and/or activity of a transcript and/or an encoded product thereby, and a provided technology can increase level and/or activity of a transcript and/or an encoded product thereby, e.g., through introducing one or more A to I to a transcript. In some embodiments, a condition, disorder or disease is associated with splicing, and a provided technology provides splicing modulation through introducing one or more A to I to a transcript (e.g., pre-mRNA).

In some embodiments, oligonucleotide compositions in provided methods are chirally controlled oligonucleotide compositions. In some embodiments, a method of treating a condition, disorder or disease can include administering a composition comprising a plurality of oligonucleotides sharing a common base sequence, which base sequence is complementary to a target sequence in a target transcript. Among other things, the present disclosure provides an improvement that comprises administering as the oligonucleotide composition a chirally controlled oligonucleotide composition as described in the present disclosure, characterized in that, when it is contacted with the target transcript in a system, adenosine editing of the transcript is improved relative to that observed under a reference condition selected from the group consisting of absence of the composition, presence of a reference composition, and any combinations thereof. In some embodiments, a reference composition is a racemic preparation of oligonucleotides of the same sequence or constitution. In some embodiments, a target transcript is an oligonucleotide transcript.

As appreciated by those skilled in the art, among other things, provided technologies can be utilized for various applications which involve and/or can benefit from an adenosine to inosine conversion. Certain applications are described in below.

| | | Treatment Modality | | |
|---|---|---|---|---|
| | | oligonucleotide- | oligonucleotide/ | |
| | | mediated | siRNA-mediated | RNA |
| Application | Application | splicing | silencing | editing |
| Alter mRNA splicing | Exon | ✓ | | ✓ |
| | skipping/inclusion/ | | | |
| | restore frame | | | |
| Silence protein | Reduce levels of toxic | | ✓ | ✓ |
| expression | mRNA/protein | | | |
| Fix nonsense mutations | Restore protein | | | ✓ |
| (e.g. those that cannot be | expression | | | |
| splice-corrected) | | | | |
| Fix missense mutations | Restore protein | | | ✓ |
| (e.g., those that cannot | function | | | |
| be splice-corrected) | | | | |
| Modify amino acid | Alter protein | | | ✓ |
| codons | level/function | | | |
| Remove upstream ORF | Increase protein | | | ✓ |
| | expression | | | |

Those skilled in the art reading the present disclosure will appreciate that various G to A mutations, e.g., those in transcripts from C to T mutations, a type of the most common mutations occurring in human genes, may be corrected and thus benefit from provided technologies. In some embodiments, provided technologies may be utilized to target mutations associated with various polar or charged amino acids (e.g., Ser, Tyr, Asp, Glu, His, Asn, Gln, Lys, etc.), stop codons (opal, ochre and amber), transcriptional start sites, splicing signals, microRNA recognition sites, repetitive elements, microRNAs (miRNAs), protein encoding transcripts, etc. Among other things, provided technologies can elicit diverse functional outcomes, e.g., altered splicing, restored/improved protein expression and/or functions, etc.

In some embodiments, through editing provided technology can restore protein functions (e.g., fix nonsense and missense mutations that cannot be splice-corrected, remove stop mutations, prevent protein misfolding and aggregation, etc., and can be utilized for preventing and/or treating various conditions, disorders or diseases such as recessive or dominant genetically defined diseases), modify protein functions (e.g., alter protein processing (e.g., protease cleavage sites), protein-protein interactions, modulate signaling pathways, etc., and can be utilized for preventing and/or treating various conditions, disorders or diseases such as those related to ion channel permeability), protein upregulation (e.g., miRNA target site modification, modifying upstream ORFs, modification of ubiquitination sites, etc., and can be utilized for preventing and/or treating various conditions, disorders or diseases such as Haploinsufficient diseases)). In some embodiments, a provided technology restores or improves expression, level, function and/or activity of a protein. In some embodiments, a provided technology is useful for preventing or treating a recessive or dominant genetically defined condition, disorder or disease. e.g., one associated with a G to A mutation. In some embodiments, a condition, disorder or disease is a liver condition, disorder or disease. In some embodiments, a condition, disorder or disease is a metabolic liver condition, disorder or disease. In some embodiments, a condition, disorder or disease is a neurodevelopmental condition, disorder or disease. In some embodiments, a provided technology modify express, level, function and/or activity of a protein. In some embodiments, a provided technology reduces express, level, function and/or activity of a protein. In some embodiments, a provided technology increases express, level, function and/or activity of a protein. In some embodiments, a provided technology modulate ion channel permeability. In some embodiments, a provided technology is useful for preventing or treating a condition, disorder or disease associated with ion channel permeability. In some embodiments, a condition, disorder or disease is familial epilepsies. In some embodiments, a condition, disorder or disease is neuropathic pain. In some embodiments, a condition, disorder or disease is AATD. In some embodiments, a condition, disorder or disease is Rett syndrome. In some embodiments, a condition, disorder or disease is recessive or dominant genetically defined diseases. In some embodiments, a provided technology modifies a nucleic acid (e.g., miRNA) target site. In some embodiments, a provided technology modifies, reduces function or activity of, removes, or suppresses an upstream ORF (e.g., in some embodiments, modifies an A (e.g., of an ATG start codon of an uORF)). In some embodiments, a provided technology modifies a modification site of a protein, e.g., a ubiquitination site. In some embodiments, a provided technology is useful for preventing or treating a condition, disorder or disease associated with haploinsufficiency. In some embodiments, a provided technology is useful for preventing or treating a neuronal condition, disorder or disease. In some embodiments, a provided technology is useful for preventing or treating a neuromuscular condition, disorder or disease. In some embodiments, a provided technology is useful for preventing or treating dementias. In some embodiments, a provided technology is useful for preventing or treating dementias. In some embodiments, a provided technology is useful for preventing or treating a haploinsufficient condition, disorder or disease. In some embodiments, the provided technology provides a method for prevent or treating a condition, disorder or disease, comprising administering to a subject susceptible thereto or suffering therefrom an effective amount of an oligonucleotide or a composition thereof as described herein. Those skilled in the art appreciate that through, e.g., editing a nucleobase such as A in a RNA, a protein encoded thereby can be edited. In some embodiments, an amino acid residue is replaced with another amino acid residue. In some embodiments, a protein is elongated. In some embodiments, a protein is shortened. In some embodiments, expression, level, function, stability, property and/or activity are modulated. In some embodiments, some properties and/or activities are enhanced while others are reduced or maintained the same. In some embodiments, some properties and/or activities are reduced while others are enhanced or maintained the same.

In some embodiments, provided technology edits a nucleic acid or a codon comprising a mutation. In some embodiments, a mutation is a nonsense mutation. In some embodiments, a mutation is a missense mutation. In some embodiments, a mutation is a silent mutation. In some embodiments, a provided technology fixes a nonsense mutation. In some embodiments, a provided technology fixes a missense mutation. In some embodiments, a provided technology removes a stop mutation. In some embodiments, a provided technology prevents or reduces misfolding and/or aggregation. In some embodiments, a provided technology edits a codon comprising a mutation. In some embodiments, an edited nucleobase is a mutation. In some embodiments, an edited nucleobase is not a mutation but another nucleobase in a codon. In some embodiments, after editing a codon becomes its corresponding wild type codon. In some embodiments, after editing a codon encodes the same amino acid as a wild type codon. In some embodiments, after editing a codon encodes a different amino acid from a wild type codon. In some embodiments, a protein comprising such a different amino acid residue shares one or more properties and/or performs one or more functions of its corresponding wild type protein. In some embodiments, a protein comprising such a different amino acid residue shares more similarities to a wild type protein, and/or provides higher levels of desired activities compared to a corresponding mutated, un-edited protein. In some embodiments, a nonsense or missense mutation cannot be splice-corrected. In some embodiments, a provided technology creates a silent mutation. In some embodiments, a silent mutation modulates levels of an encoded protein. In some embodiments, a protein level is increased. In some embodiments, a protein level is decreased.

In some embodiments, a provided technology modifies protein function. In some embodiments, a provided technology changes one or more properties and/or functions of a nucleic acid (e.g., a transcript) and/or a protein. In some embodiments, a provided technology increases, promotes, or enhances one or more properties and/or functions of a nucleic acid (e.g., a transcript) and/or a protein. In some embodiments, a provided technology provide one or more new properties and/or activities, e.g., of a nucleic acid (e.g., a transcript) and/or a protein. In some embodiments, a provided technology decreases, inhibits, or removes one or more properties and/or functions of a nucleic acid (e.g., a transcript) and/or a protein. In some embodiments, a provided technology alter protein processing. For example, in some embodiments, protease cleavage sites are edited. In some embodiments, provided technologies edit one or more residues involved in protein-protein interactions. In some embodiments, provided technologies edit amino acid residues at protein-protein interactions domains. In some embodiments, through editing mRNAs that encode proteins, residues at various regions of polypeptides, e.g., protease cleavage sites, various domains (e.g., protein-protein interactions domains), modification sites, miRNA targeting sites, ubiquitination sites, etc. can be edited. In some embodiments, provided technologies modulate signaling pathways.

In some embodiments, provided technologies restore, increase or enhance levels of functional proteins. In some embodiments, provided technologies reduce levels and/or activities of mutant or undesired nucleic acids (e.g., RNA transcripts) and proteins. In some embodiments, provided technologies restore or correct expression of one or more polypeptides. In some embodiments, provided technologies can upregulate expression. In some embodiments, provided technologies can upregulate translation. In some embodiments, provided technologies can upregulate activity levels of polypeptides. In some embodiments, provided technologies modify functions of target nucleic acids (e.g., RNA transcripts) and/or products encoded thereby (e.g., polypeptides). In some embodiments, provided technologies modulate post-translation modifications of target nucleic acids (e.g., RNA transcripts) and/or products encoded thereby (e.g., polypeptides). In some embodiments, provided technologies can upregulate levels of polypeptides. In some embodiments, provided technologies edit codons encoding amino acid residues involved in protein-protein interactions or protein interactions with other agents, including in some embodiments, changing the amino acid residues to different amino acid residues to enhance or reduce interactions. In some embodiments, provided technologies modify one or more functions of nucleic acids and/or proteins. In some embodiments, provided technologies can modulate protein-protein interactions. In some embodiments, provided technologies edit encoding transcripts to remove, change, or incorporate amino acid residues for post-translation modification. In some embodiments, provided technologies modulate post-translational modifications. In some embodiments, provided technologies modulate nucleic acid folding. In some embodiments, provided technologies modulate protein folding. In some embodiments, provided technologies modulate stability of transcripts and/or products thereof. In some embodiments, provided technologies modulate protein stability. In some embodiments, provided technologies modulate processing of transcripts and/or products thereof In some embodiments, provided technologies modulate nucleic acids (e.g., transcripts) processing. In some embodiments, provided technologies alter protein processing. In some embodiments, provided technologies modulate post translational processes. For example, in some embodiments, provided technologies modulate PCSK9 post translational processes. Among other things, provided technologies are applicable to a wide range of therapeutic applications with large patient populations.

For example, in some embodiments, one or more amino acid residues of one or more proteins may be changed through editing of encoding mRNAs to modulate protein-protein interactions. Suitable amino acid residues for editing include various reported amino acid residues involved in protein-protein interactions, or can be identified through technologies available in the art, e.g., mutation technologies, structural biology technologies, etc. In some embodiments, the present disclosure provides technologies for modulating levels, properties and/or activities of nucleic acids (e.g., transcripts) and/or proteins through editing of nucleic acids (e.g., transcripts) and/or proteins that interact them. In some embodiments, the present disclosure provides technologies for modulating levels and/or activities of a protein (e.g., a transcription factor) and/or transcription and/or expression regulated thereby. In some embodiments, a provided technology comprises editing an amino acid residue of a protein (e.g., a transcription factor) or a partner protein that it interacts with, wherein interaction between the protein and a partner protein is reduced or enhanced. In some embodiments, a provided technology comprises editing an amino acid residue of a protein (e.g., a transcription factor) or a partner protein that it interacts with, wherein interaction between the protein and a partner protein is reduced. In some embodiments, such editing stabilizes a protein so that its levels and/or activities (e.g., transcription activation of certain nucleic acids) are increased. In some embodiments, the present disclosure provides technologies for modulating (e.g., activating, increasing, reducing, suppressing, etc.) expression of a nucleic acid, comprising editing an adenosine in a transcript encoding a protein that regulates expression of the nucleic acid, or a protein that interacts with a protein that regulates expression of the nucleic acid, or a protein that is a member of a pathway comprising a protein that regulates expression of the nucleic acid, wherein editing modulates levels and/or activities of a protein that regulates expression of the nucleic acid. In some embodiments, transcripts levels and/or activities of a nucleic acids are modulated. In some embodiments, levels and/or activities of proteins encoded by such transcripts are modulated. Among other things, the present disclosure confirms that many functions, activities, pathways, etc., that involve protein-protein interactions may be modulated through editing of interacting amino acid residues of one or more interacting proteins. For example, editing of one or more amino acid residues in NRF2 (e.g., Glu82 (e.g., to Gly), Glu79 (e.g., to Gly), Glu78 (e.g., to Gly), Asp76 (e.g., to Gly), Ile28 (to Val), Asp27 (e.g., to Gly), Gln26 (e.g., to Arg), etc.) or Keap1 (e.g., Ser603 (e.g., to Gly), Tyr572 (e.g., to Cys), Tyr525 (e.g., to Cys), Ser508 (e.g., to Gly), His436 (e.g., to Arg), Asn382 (e.g., to Asp), Arg380 (e.g., to Gly), Tyr334 (e.g., to Cys), etc.) can increase levels and/or activities of NRF2, and/or expression of various nucleic acids (e.g., various genes) regulated by NRF2. In some embodiments, the present disclosure provides a method for modulating, e.g., reducing, NRF2-Keap1 interaction in a system, comprising administering to a system comprising a NRF2 or Keap1 mRNA an oligonucleotide or a composition thereof, wherein the oligonucleotide edits an adenosine in the mRNA so that an amino acid residue in a protein encoded by the mRNA is edited to be a different residue. In some embodiments, the present disclosure provides a method for increasing a level and/or activity of NRF2 in a system, comprising administering to a system comprising a NRF2 or Keap1 mRNA an oligonucleotide or a composition thereof, wherein the oligonucleotide edits an adenosine in the mRNA so that an amino acid residue in a protein encoded by the mRNA is edited to be a different residue. In some embodiments, the present disclosure provides a method for increasing transcription or expression of a NRF2-regulated nucleic acid (e.g., a gene), comprising administering to a system comprising a NRF2 or Keap1 mRNA an oligonucleotide or a composition thereof, wherein the oligonucleotide edits an adenosine in the mRNA so that an amino acid residue in a protein encoded by the mRNA is edited to be a different residue. In some embodiments, levels and/or activities of transcripts from NRF2-regulated nucleic acids, e.g., genes such as SRGN, HMOX1, SLC7a11, NQO1, etc., and/or products (e.g., proteins) encoded thereby are increased. In some embodiments, a system comprising a NRF2 and a Keap1 mRNA, and NRF2 and Keap1 proteins are translated from such mRNA. In some embodiments, a target adenosine of a NRF2 and/or a Keap1 mRNA is edited so that an amino acid residue is replaced with a different amino acid residue after translation. In some embodiments, an administered oligonucleotide or composition thereof targets NRF2 mRNA. In some embodiments, an administered oligonucleotide or composition thereof targets Keap1 mRNA. In some embodiments, an amino acid residue in NRF2 (e.g., Glu82 (e.g., to Gly), Glu79 (e.g., to Gly), Glu78 (e.g., to Gly), Asp76 (e.g., to Gly), Ile28 (to Val), Asp27 (e.g., to Gly), Gln26 (e.g., to Arg), etc.) is edited. In some embodiments, an amino acid residue in Keap1 (e.g., Ser603 (e.g., to Gly), Tyr572 (e.g., to Cys), Tyr525 (e.g., to Cys), Ser508 (e.g., to Gly), His436 (e.g., to Arg), Asn382 (e.g., to Asp), Arg380 (e.g., to Gly), Tyr334 (e.g., to Cys), etc.) is edited. In some embodiments, two or more amino acid residues are edited. In some embodiments, each edited amino acid residue is independently a NRF2 residue. In some embodiments, each edited amino acid residue is independently a Keap1 residue. In some embodiments, an edited amino acid residue is a Keap1 residue, and an edited amino acid residue is a NRF2 residue. In some embodiments, a system is or comprises a cell. In some embodiments, a system is or comprises a tissue. In some embodiments, a system is or comprises an organ. In some embodiments, a system is an orgasm. In some embodiments, a system is an in vitro system. Certain NRF2-targeting and Keap1-targeting oligonucleotides and/or oligonucleotide compositions are presented in the Table(s) as examples. In some embodiments, provided technologies are useful for treating a condition, disorder or disease related to NRF2. In some embodiments, provided technologies are useful for treating a condition, disorder or disease related to Keap1. In some embodiments, provided technologies are useful for treating a condition, disorder or disease related to NRF2-Keap1 interaction.

In some embodiments, provided technologies modulate enzymatic activities. In some embodiments, provided technologies increase an enzymatic activity, e.g., through editing a codon to a codon encoding a amino acid residue that can increase an enzymatic activity. In some embodiments, provided technologies decrease an enzymatic activity, e.g., those associated with a condition, disorder or disease, through editing a codon to a codon encoding a amino acid residue that can decrease an enzymatic activity. Various enzymatic activities, in many cases with amino acid residues involved for such activities, are reported or can be identified and characterized, and can be modulated in accordance with the present disclosure. In some embodiments, an activity is a kinase activity.

In some embodiments, editing of a protein (e.g., through editing of its encoding mRNA to change one or more amino acid residues) decreases degradation of the protein or a protein which it interacts with. In some embodiments, editing of a protein upregulate its levels. In some embodiments, editing of a protein modulate protein processing. In some embodiments, editing of a protein modulate its folding. In some embodiments, editing of a protein modulate its stability. In some embodiments, editing of a protein modulate protein modification (e.g., increasing, decreasing, removing or introducing a modification site, etc.). In some embodiments, editing of a protein modulate post-translational modification (e.g., increasing, decreasing, removing or introducing a modification site, etc.). In some embodiments, provided technologies are useful for treating associated conditions, disorders or diseases, such as dementias, familial epilepsies, neuropathic pain, neuromuscular disorders, dementias, haploinsufficient diseases, loss of function conditions, disorders or diseases, etc.

Technologies of present disclosure can provide efficient editing in various types of cells, tissues, organs and/or organisms. In some embodiments, provided technologies can provide efficient editing in various immune cells. In some embodiments, provided technologies can provide high levels of editing in human peripheral blood mononuclear cells (PBMCs). Among other things, provided technologies can provide high levels of editing in various cell populations such as CD4+ T cells, CD8+ T cells, CD14 monocytes, CD19 B cells, NK cells, Tregs T cells, etc. In some embodiments, immune cells are activated (e.g., by PHA) before contact with oligonucleotides. In some embodiments, cells are non-activated. In some embodiments, similar levels of editing are obtained in activated and non-activated cells. In some embodiments, higher levels of editing are obtained in activated cells. In some embodiments, after editing cells, e.g., PBMCs, may be sorted into various cell types. In some embodiments, cells can be first sorted before contact with oligonucleotides. As appreciated by those skilled in the art, immune cells have a number of functions and may be utilized for a number of purposes including for treating various conditions, disorders or diseases. In some embodiments, immune cells are utilized in immunotherapy, e.g., for various types of cancer. Among other things, the present disclosure provides technologies for editing one or more transcripts expressed in immune cells to improve its properties and/or activities for immunotherapy. In some embodiments, provided technologies can reduce expression and/or activity of one or more genes in immune cells, e.g., FAS, BID, CTLA4, PDCD1, CBLB, PTPN6, TRAC, TRBC, etc. In some embodiments, transcripts from such genes are edited. In some embodiments, a target cell is a T cell, e.g., a CD8+ T cell (e.g., a CD8+ naïve T cell, central memory T cell, or effector memory T cell), a CD4+ T cell, a natural killer T cell (NK T cell), a regulatory T cell (Treg), a stem cell memory T cell, a lymphoid progenitor cell, a hematopoietic stem cell, a natural killer cell (NK cell) or a dendritic cell. In some embodiments, cells are CD4+ cells, e.g., CD4+ T cells. In some embodiments, cells are CD8+ cells, e.g., CD8+ T cells. In some embodiments, cells are CD14+ cells, e.g., CD14+ monocytes. In some embodiments, cells are CD19+ cells, e.g., CD19+ B cells. In some embodiments, cells are NC cells. In some embodiments, cells are T-regulatory cells. In some embodiments, a target cell is an induced pluripotent stem (iPS) cell or a cell derived from an iPS cell, e.g., an iPS cell generated from a subject, manipulated to alter (e.g., induce a mutation in) expression of one or more genes, e.g., FAS, BID, CTLA4, PDCD1, CBLB, PTPN6, TRAC or TRBC gene, and differentiated into, e.g., a T cell, e.g., a CD8+ T cell (e.g., a CD8+ naïve T cell, central memory T cell, or effector memory T cell), a CD4+ T cell, a stem cell memory T cell, a lymphoid progenitor cell or a hematopoietic stem cell.

Among other things, provided technologies are useful for increasing, enhancing, improving or upregulating levels, properties, activities, etc., of various polypeptides including various proteins. In some embodiments, provided technologies modify binding or target sites, e.g., miRNA target sites. In some embodiments, provided technologies modify regulatory elements in transcripts. In some embodiments, provided technologies modify upstream ORFs (e.g., A in ATG). In some embodiments, provided technologies modify amino acid residues that can be modified, e.g., ubiquitination sites. Those skilled in the art appreciate provided technologies can also be useful for decreasing or downregulating levels, properties, activities, etc., of various polypeptides including various proteins through modifying RNAs.

In some embodiments, an editing site, e.g., a target adenosine, is in a coding region. In some embodiments, it is in a non-coding region. In some embodiments, a target nucleic acid is a non-coding RNA.

Certain applications, including various conditions, disorders or diseases that can be prevented or treated, are described, e.g., in WO 2016/097212, WO 2017/220751, WO 2018/041973, WO 2018/134301A1, WO 2020/154342, WO 2020/154343, WO 2020/154344, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256.

Many adenosines associated with various conditions, disorders or diseases are reported or can be identified, and can be targeted using provided technologies, e.g., for preventing or treating associated conditions, disorders or diseases. For example, it has been reported that various adenosines associated with various conditions, disorders or diseases have been identified in SNCA (e.g., Parkinson's disease), APP (e.g., Alzheimer's Disease), Tau (e.g., Alzheimer's Disease), Nav1.7 (e.g., Chronic Pain), C9orf72 (e.g., Amyotrophic Lateral Sclerosis), SOD1 (e.g., Amyotrophic Lateral Sclerosis), DYRK1A (e.g., Down Syndrome), IT15 (e.g., Huntington's Disease), HEXA (e.g., Tay-Sachs Disease), RAI1 (e.g., Protocki-Lupski Syndrome), ABCA4 (e.g., Stargardt Disease), USH2A (e.g., Usher Syndrome), NRP1 (e.g., Wet AMD, Dry AMD, etc.), PCSK9 (e.g., cardiovascular conditions, disorders or diseases), LIPA (e.g., Cholesteryl Ester Storage Disease), HFE (e.g., Hemochromatosis), ALAS1 (e.g., Porphyria/Acute Hepatic Porphyria), ATP7B (Wilson Disease), COL4A5 (e.g., Alport Syndrome), LDHA (e.g., Primary Hyperoxaluria), HAO1 (e.g., Primary Hyperoxaluria Type 2), DUX4 (e.g., Facioscapulohumeral Dystrophy), DMPK (e.g., Myotonic Dystrophy), BCL11A (e.g., Sickle Cell Disease), Mex3B (e.g., Asthma), CIDEC (e.g., obesity), SCD1 (e.g., obesity), GNB3 (e.g., obesity), FGFR3 (e.g., Achondroplasia), CLCN7 (e.g., Osteopetrosis), PMP22 (e.g., Charcot-Marie-Tooth Disease), ENAC (e.g., Cystic Fibrosis), GHR (e.g., Acromegaly), TTR (e.g., Transthyretin Amyloidosis (familial)), etc. In some embodiments, the present disclosure provides oligonucleotides and compositions targeting such adenosines, and methods for preventing or treating such conditions, disorders or diseases.

In some embodiments, conditions, disorders or diseases that may be treated include, for example, , alpha-1 antitrypsin deficiency, Alzheimer's disease, amyloid diseases, Becker muscular dystrophy, breast cancer predisposition mutations, Canavan disease, Charcot-Marie-Tooth disease, cystic fibrosis, Factor V Leiden deficiency. Type 1 diabetes, Type 2 diabetes, Duchenne muscular dystrophy, Fabry disease, hereditary tyrosinemia type I (HTI), familial adenomatous polyposis, familial amyloid cardiomyopathy, familial amyloid polyneuropathy, familial dysautonomia, familial hypercholesterolemia, Friedreich's ataxia, Gaucher disease type I, Gaucher disease II, glycogen storage disease type II, GM2 gangliosidosis, hemochromatosis, hemophilia A, hemophilia B, hemophilia C, hexosaminidase A deficiency, ovarian cancer predisposition mutations, obesity, phenylketonuria, polycystic kidney disease, prion disease, senile systemic amyloidosis, sickle-cell disease, Smith-Lemli-Opitz syndrome, spinal muscular atrophy, Wilson's disease, Parkinson's disease, and hereditary blindness. In some embodiments, diseases/targets include: cystic fibrosis transmembrane conductance regulator gene (CFTR); albinism, Amyotrophic lateral sclerosis, Asthma, 3-thalassemia, Cadasil syndrome, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, dystrophin gene (DMD); amyloid beta (A4) precursor protein gene (APP); Factor V Leiden associated disorders, Glucose-6-phosphate dehydrogenase. Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Myotonic dystrophy types I and II, neurofibromatosis, Niemann-Pick disease type A, B and C, NY-eso1 related cancer, Rett syndrome, NY-ESO-1 related cancer, 11-thalassemia, Galactosemia, Gaucher's Disease, Factor XII gene; Factor IX gene; Factor XI gene; HgbS; insulin receptor gene; adenosine deaminase gene; alpha-I antitrypsin gene; breast cancer 1 gene (BRCA1); breast cancer 2 gene (BRCA2); aspartocyclase gene (ASPA); galactosidase alpha gene (GLA); adenomatous polyposis coli gene (APC); inhibitor of kappa light polypeptide gene enhancer in B-cells, kinase complex-associated protein (IKBKAP); glucosidase beta acid gene (GBA); glucosidase alpha acid gene (GAA); hemochromatosis gene (HFE); apolipoprotein B gene (APOB); low density lipoprotein receptor gene (LDLR), low density lipoprotein receptor adaptor protein 1 gene (LDLRAP1); proprotein convertase subtilisin/kexin type 9 gene (PCSK9); polycystic kidney disease I (autosomal dominant) gene (PKD-1); Prion protein gene (PRNP); PTP-1B; 7-dehydrocholesterol reductase gene (DHCR7); survival of motor neuron 1, telomeric gene (SMN1); biquitin-like modifier activating enzyme 1 gene (UBA1); dynein, cytoplasmic 1, heavy chain 1 gene (DYNC1H1), survival of motor neuron 2, centromeric gene (SMN2); (vesicle-associated membrane protein)-associated protein B and C (VAPB); hexosaminidase A (alpha polypeptide) gene (HEXA); transthyretin gene (TTR); ATPase, Cu++ transporting, beta polypeptide gene (ATP7B); phenylalanine hydroxylase gene (PAH); rhodopsin gene; retinitis pigmentosa 1 (autosomal dominant) gene (RP1); retinitis pigmentosa 2 (X-linked recessive) gene (RP2), Sturge-Weber Syndrome, Parkinson's disease, Peutz-Jeghers Syndrome, Pompe's disease, Primary Ciliary Disease, Prothrombin mutation related disorders, such as the Prothrombin G20210A mutation, Pulmonary Hypertension, Sandhoff Disease, Severe Combined Immune Deficiency Syndrome (SCID), Stargardt's Disease, Tay-Sachs Disease, Usher syndrome, X-linked immunodeficiency, various forms of cancer (e.g. BRCA1 and 2 linked breast cancer and ovarian cancer), and the like and other known gene targets. Other diseases include those point mutations or small deletions or insertions or diseases that can be corrected by point changes or small deletions or insertions listed in http://www.omim.org/Online Mendelian Inheritance in Man* An Online Catalog of Human Genes and Genetic Disorders Updated, e.g., on 24 Sep. 2021.

In some embodiments, the present disclosure provides technologies targeting IDUA. In some embodiments, the present disclosure provides methods for preventing or treating a condition, disorder or disease associated with IDUA, comprising administering to a subject susceptible thereto or suffering therefrom an effective amount of an oligonucleotide or composition. In some embodiments, a subject benefits from a G to A editing in IDUA. In some embodiments, a condition, disorder or disease is Hurler syndrome. In some embodiments, the present disclosure provides technologies targeting PINK1. In some embodiments, the present disclosure provides methods for preventing or treating a PINK1-associated condition, disorder or disease, comprising administering to a subject susceptible thereto or suffering therefrom an effective amount of an oligonucleotide or composition. In some embodiments, a subject benefits from a G to A editing in PINK1. In some embodiments, a condition, disorder or disease is Parkinson's disease. In some embodiments, the present disclosure provides technologies targeting Factor V Leiden. In some embodiments, the present disclosure provides methods for preventing or treating a Factor V Leiden-associated condition, disorder or disease, comprising administering to a subject susceptible thereto or suffering therefrom an effective amount of an oligonucleotide or composition. In some embodiments, a subject benefits from a G to A editing in Factor V Leiden. In some embodiments, a condition, disorder or disease is Factor V Leiden deficiency. In some embodiments, the present disclosure provides technologies targeting CFTR. In some embodiments, the present disclosure provides methods for preventing or treating a CFTR-associated condition, disorder or disease, comprising administering to a subject susceptible thereto or suffering therefrom an effective amount of an oligonucleotide or composition. In some embodiments, a subject benefits from a G to A editing in CFTR. In some embodiments, a condition, disorder or disease is cystic fibrosis.

It is reported that there are over 32,000 pathogenic human SNPs nearly half of which are G to A mutations that can be corrected by provided technologies. Indeed, tens of thousands of disease are reported to be associated with G to A mutation and can be prevented or treated by provided technologies. Among other things, provided technologies can be utilized to prevent or treat many conditions, disorders or diseases associated with premature stop codons; it is reported that ˜12% of all reported disease-causing mutations are single point mutations that result in a premature stop codon. In some embodiments, the provided technologies correct a premature stop codon. See, e.g., ClinVar database; Gaudelli NM et al., Nature. 2017 Nov. 23; 551(7681): 464-471; Keeling KM et al., Madame Curie Bioscience Database 2000-2013; etc.

In some embodiments, when an oligonucleotide or oligonucleotide composition is contacted with a target nucleic acid comprising a target adenosine in a system, a target adenosine in a target nucleic acid is modified. In some embodiments, when an oligonucleotide or oligonucleotide composition is contacted with a target nucleic acid comprising a target adenosine in a system, level of a target nucleic acid is reduced compared to absence of the product or presence of a reference oligonucleotide. In some embodiments, when an oligonucleotide or oligonucleotide composition is contacted with a target nucleic acid comprising a target adenosine in a system, splicing of a target nucleic acid or a product thereof is altered compared to absence of the oligonucleotide or presence of a reference oligonucleotide. In some embodiments, when an oligonucleotide or oligonucleotide composition is contacted with a target nucleic acid comprising a target adenosine in a system, level of a product of a target nucleic acid is altered compared to absence of the product or presence of a reference oligonucleotide. In some embodiments, level of a product is increased, wherein the product is or is encoded by a nucleic acid which is otherwise identical to a target nucleic acid but a target adenosine is modified. In some embodiments, level of a product is increased, wherein the product is or is encoded by a nucleic acid which is otherwise identical to a target nucleic acid but a target adenosine is replaced with inosine. In some embodiments, level of a product is increased, wherein the product is or is encoded by a nucleic acid which is otherwise identical to a target nucleic acid but the adenine of a target adenosine is replaced with guanine. In some embodiments, a product is a protein. In some embodiments, a target adenosine is a mutation from guanine. In some embodiments, a target adenosine is more associated with a condition, disorder or disease than a guanine at the same position. In some embodiments, an oligonucleotide is capable of forming a double-stranded complex with a target nucleic acid. In some embodiments, a target nucleic acid or a portion thereof is or comprises RNA. In some embodiments, a target adenosine is of an RNA. In some embodiments, a target adenosine is modified, and the modification is or comprises deamination of a target adenosine. In some embodiments, a target adenosine is modified and the modification is or comprises conversion of a target adenosine to an inosine. In some embodiments, a modification is promoted by an ADAR protein. In some embodiments, a system is an in vitro or ex vivo system comprising an ADAR protein. In some embodiments, a system is or comprises a cell that comprises or expresses an ADAR protein. In some embodiments, a system is a subject comprising a cell that comprises or expresses an ADAR protein. In some embodiments, a ADAR protein is ADAR1. In some embodiments, an ADAR1 protein is or comprises p110 isoform. In some embodiments, an ADAR1 protein is or comprises p150 isoform. In some embodiments, an ADAR1 protein is or comprises p110 and p150 isoform. In some embodiments, a ADAR protein is ADAR2. In some embodiments, the present disclosure among other things provides technologies for recruiting enzymes to target sites (e.g., those comprising target As), comprising contacting such target sites with, or administering to systems comprising or expressing polynucleotide (e.g., RNA) comprising such target sites, provided oligonucleotides or compositions thereof In some embodiments, an enzyme is an RNA-editing enzyme such as ADAR1, ADAR2, etc. as described herein.

In some embodiments, an oligonucleotide composition comprising a plurality of oligonucleotides provide a greater level, e.g., a target adenosine is modified at a greater level, than that is observed with a comparable reference oligonucleotide composition. In some embodiments, a reference oligonucleotide composition comprises no or a lower level of oligonucleotides of the plurality. In some embodiments, a reference composition does not contain oligonucleotides that have the same constitution as an oligonucleotide of the plurality. In some embodiments, a reference composition does not contain oligonucleotides that have the same structure as an oligonucleotide of the plurality. In some embodiments, a reference oligonucleotide composition is a composition whose oligonucleotides having the same base sequence as oligonucleotides of the plurality contain a lower level of 2′-F modifications compared to oligonucleotides of the plurality. In some embodiments, a reference oligonucleotide composition is a composition whose oligonucleotides having the same base sequence as oligonucleotides of the plurality contain a lower level of 2′-OMe modifications compared to oligonucleotides of the plurality. In some embodiments, a reference oligonucleotide composition is a composition whose oligonucleotides having the same base sequence as oligonucleotides of the plurality have a different sugar modification pattern compared to oligonucleotides of the plurality. In some embodiments, a reference oligonucleotide composition is a composition whose oligonucleotides having the same base sequence as oligonucleotides of the plurality contain a lower level of modified internucleotidic linkages compared to oligonucleotides of the plurality. In some embodiments, a reference oligonucleotide composition is a composition whose oligonucleotides having the same base sequence as oligonucleotides of the plurality contain a lower level of phosphorothioate internucleotidic linkages compared to oligonucleotides of the plurality. In some embodiments, a composition is a stereorandom oligonucleotide composition. In some embodiments, a reference composition is a stereorandom oligonucleotide composition of oligonucleotides of the same constitution as oligonucleotides of the plurality.

In some embodiments, the present disclosure provides technologies for modifying a target adenosine in a target nucleic acid, comprising contacting a target nucleic acid with an provided oligonucleotide or oligonucleotide composition as described herein. In some embodiments, the present disclosure provides a method for deaminating a target adenosine in a target nucleic acid, comprising contacting a target nucleic acid with an oligonucleotide or composition as described herein. In some embodiments, the present disclosure provides a method for producing, or restoring or increasing level of a product of a particular nucleic acid, comprising contacting a target nucleic acid with a provided oligonucleotide or composition wherein a target nucleic acid comprises a target adenosine, and the particular nucleic acid differs from a target nucleic acid in that the particular nucleic acid has an I or G instead of a target adenosine. In some embodiments, the present disclosure provides a method for reducing level of a product of a target nucleic acid, comprising contacting a target nucleic acid with an oligonucleotide or composition of the present disclosure, wherein a target nucleic acid comprises a target adenosine. In some embodiments, a product is a protein. In some embodiments, a product is a mRNA.

In some embodiments, the present disclosure provides a method, comprising:
contacting an oligonucleotide or composition with a sample comprising a target nucleic acid and an adenosine deaminase, wherein:
the base sequence of the oligonucleotide or oligonucleotides in the oligonucleotide composition is substantially complementary to that of a target nucleic acid; and
a target nucleic acid comprises a target adenosine;
wherein a target adenosine is modified.

In some embodiments, the present disclosure provides a method comprising:
1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise more sugars with 2′-F modification, more sugars with 2′-OR modification wherein R is not —H, and/or more chiral internucleotidic linkages than oligonucleotides of the reference plurality; and
the first oligonucleotide composition provides a higher level of modification compared to oligonucleotides of the reference oligonucleotide composition.

In some embodiments, the present disclosure provides a method comprising:
obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise more sugars with 2′-F modification, more sugars with 2′-OR modification wherein R is not —H, and/or more chiral internucleotidic linkages than oligonucleotides of the reference plurality.

In some embodiments, the present disclosure provides a method comprising:
1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise more sugars with 2′-F modification, more sugars with 2′-OR modification wherein R is not —H, and/or more chirally controlled chiral internucleotidic linkages than oligonucleotides of the reference plurality; and
the first oligonucleotide composition provides a higher level of modification compared to oligonucleotides of the reference oligonucleotide composition.

In some embodiments, the present disclosure provides a method comprising:
obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise more sugars with 2′-F modification, more sugars with 2′-OR modification wherein R is not —H, and/or more chirally controlled chiral internucleotidic linkages than oligonucleotides of the reference plurality.

In some embodiments, the present disclosure provides a method comprising:
1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise one or more chirally controlled chiral internucleotidic linkages; and
oligonucleotides of the reference plurality comprise no chirally controlled chiral internucleotidic linkages (a reference oligonucleotide composition is a “stereorandom composition); and
the first oligonucleotide composition provides a higher level of modification compared to oligonucleotides of the reference oligonucleotide composition.

In some embodiments, the present disclosure provides a method comprising:
obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and
wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;
wherein:
oligonucleotides of the first plurality comprise one or more chirally controlled chiral internucleotidic linkages; and
oligonucleotides of the reference plurality comprise no chirally controlled chiral internucleotidic linkages (a reference oligonucleotide composition is a “stereorandom composition).

In some embodiments, a first oligonucleotide composition is an oligonucleotide composition as described herein. In some embodiments, a first oligonucleotide composition is a chirally controlled oligonucleotide composition. In some embodiments, a deaminase is an ADAR enzyme. In some embodiments, a deaminase is ADAR1. In some embodiments, a deaminase is ADAR2. In some embodiments, a sample is or comprise a cell. In some embodiments, a target nucleic acid is more associated with a condition, disorder or disease, or decrease of a desired property or function, or increase of an undesired property or function, compared to a nucleic acid which differs from a target nucleic acid in that it has an I or G at the position of a target adenosine instead of a target adenosine. In some embodiments, a target adenosine is a G to A mutation.

Among other things, oligonucleotide designs of the present disclosure, e.g., nucleobase, sugar, internucleotidic linkage modifications, control of linkage phosphorus stereochemistry, and/or patterns thereof, can be applied to improve prior technologies. In some embodiments, the present disclosure provides improvement over prior technologies by introducing one or more structural features of the present disclosure, e.g., nucleobase, sugar, internucleotidic linkage modifications, control of linkage phosphorus stereochemistry, and/or patterns thereof to oligonucleotides in prior technologies. In some embodiments, an improvement is or comprises improvement from control of linkage phosphorus stereochemistry.

In some embodiments, the present disclosure provides technologies for improving adenosine editing by a polypeptide, e.g., ADAR1, ADAR2, etc., comprising incorporating into an oligonucleotide a design (e.g., one or more modifications and/or patterns thereof) as described herein. In some embodiments, a design is or comprises a modified base as described herein, e.g., at the position opposite to a target adenosine and/or one or both of its neighboring positions. In some embodiments, a design is or comprises one or more sugar modifications and/or patterns thereof, one or more base modifications and/or patterns thereof, one or more modified internucleotidic linkages and/or patterns thereof, and/or controlled stereochemistry at one or more positions and/or patterns thereof In some embodiments, a provided technology improves editing by ADAR1 more than ADAR2. In some embodiments, a provided technology improves editing by ADAR2 more than ADAR1. In some embodiments, a provided technology improves editing by ADAR1 p110 more than p150 (e.g., in some embodiments, Rp (e.g., of phosphorothioate internucleotidic linkages) at one or more positions). In some embodiments, a provided technology improves editing by ADAR1 p150 more than p110.

In some embodiments, a provided technology comprises increasing levels of an adenosine editing polypeptide, e.g., ADAR1 (p110 or p150) or ADAR2, or a portion thereof. In some embodiments, an increase is through expression of an exogenous of a polypeptide.

In some embodiments, a provided oligonucleotide or oligonucleotide composition does not cause significant degradation of a nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)). In some embodiments, a composition does not cause significant undesired exon skipping or altered exon inclusion in a target nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).

In some embodiments, provided technologies can provide high levels of adenosine editing (e.g., conversion to inosine). In some embodiments, percentage of target adenosine editing is about 10%-100%, e.g., at least about 10%, 15%, 0% 25%, 30%, 35%, 40%, 45%, 50%, 60%, 70%, 75%, 80%, 85%, 90%, or 95%. In some embodiments, it is at least 10%. In some embodiments, it is at least 15%. In some embodiments, it is at least 20%. In some embodiments, it is at least 25%. In some embodiments, it is at least 30%. In some embodiments, it is at least 35%. In some embodiments, it is at least 40%. In some embodiments, it is at least 45%. In some embodiments, it is at least 50%. In some embodiments, it is at least 60%. In some embodiments, it is at least 70%. In some embodiments, it is at least 75%. In some embodiments, it is at least 80%. In some embodiments, it is at least 85%. In some embodiments, it is at least 90%. In some embodiments, it is at least 95%. In some embodiments, it is at least about 100%.

In some embodiments, an oligonucleotide or a composition thereof is capable of mediating a decrease in the expression or level of a target nucleic acid or a product thereof (e.g., by modifying a target adenosine into inosine). In some embodiments, an oligonucleotide or a composition thereof is capable of mediating a decrease in the expression or level of a target gene or a gene product thereof (e.g., by modifying a target adenosine into inosine) in a cell in vitro. In some embodiments, expression or level can be decreased by at least about 10%, 15%, 20% 25% 30% 35%, 40%, 45%, 50%, 60%, 70%, 75%, 80%, 85%, 90%, or 95%. In some embodiments, expression or level of a target gene or a gene product thereof can be decreased by at least about 10%, 15%, 20%, 25%, 30%, 35%, 40%, 45%, 50%, 60%, 70%, 75%, 80%, 85%, 90%, or 95% by ADAR-mediated deamination directed by an oligonucleotide or a composition thereof, e.g., at a concentration of 10 uM or less in a cell(s) in vitro. In some embodiments, an oligonucleotide or a composition thereof is capable of provide suitable levels of activities at a concentration of 1 nM, 5 nM, 10 nM or less (e.g., when assayed in cells in vitro or in vivo).

In some embodiments, activity of provided oligonucleotides and compositions may be assessed by IC50, which is the inhibitory concentration to decrease level of a target nucleic acid or a product thereof by 50% in a suitable condition, e.g., cell-based in vitro assays. In some embodiments, provided oligonucleotides or compositions have an IC50 no more than 0.001, 0.01, 0.1, 0.5, 1, 2, 5, 10, 50, 100, 200, 500 or 1000 nM, e.g., when assessed in cell-based assays. In some embodiments, an IC50 is no more than about 500 nM. In some embodiments, an IC50 is no more than about 200 nM. In some embodiments, an IC50 is no more than about 100 nM. In some embodiments, an IC50 is no more than about 50 nM. In some embodiments, an IC50 is no more than about 25 nM. In some embodiments, an IC50 is no more than about 10 nM. In some embodiments, an IC50 is no more than about 5 nM. In some embodiments, an IC50 is no more than about 2 nM. In some embodiments, an IC50 is no more than about 1 nM. In some embodiments, an IC50 is no more than about 0.5 nM.

In some embodiments, provided technologies can provide selective editing of target adenosine over other adenosine residues in a target adenosine. In some embodiments, selectivity of a target adenosine over a non-target adenosine is at least 1.5, 2, 3, 4, 5, 6, 7, 8, 9, 10, 15, 20, 25, 30, 40, 50, 60, 70, 80, 90, 100 fold or more (e.g., as measured by level of editing of a target adenosine over a non-target adenosine at a suitable condition, or by oligonucleotide concentrations for a certain level of editing (e.g., 0.5%, 1%, 2%, 5%, 10%, 15%, 20%, 25%, 30%, 40%, 50%, etc.). In some embodiments, a selectivity is at least 2 fold. In some embodiments, a selectivity is at least 3 fold. In some embodiments, a selectivity is at least 4 fold. In some embodiments, a selectivity is at least 5 fold. In some embodiments, a selectivity is at least 10 fold. In some embodiments, a selectivity is at least 25 fold. In some embodiments, a selectivity is at least 50 fold. In some embodiments, a selectivity is at least 100 fold.

In some embodiments, the present disclosure provides a method for suppression of a transcript from a target nucleic acid sequence for which one or more similar nucleic acid sequences exist within a population, each of the target and similar sequences contains a specific characteristic sequence element that defines the target sequence relative to the similar sequences, the method comprising contacting a sample comprising transcripts of target nucleic acid sequence with an oligonucleotide, or a composition comprising a plurality of oligonucleotides sharing a common base sequence, wherein the base sequence of the oligonucleotide, or the common base sequence of the plurality of oligonucleotide, is or comprises a sequence that is complementary to the characteristic sequence element that defines the target nucleic acid sequence. In some embodiments, wherein when the oligonucleotide, or the oligonucleotide composition, is contacted with a system comprising transcripts of both the target nucleic acid sequence and a similar nucleic acid sequences, transcripts of the target nucleic acid sequence are suppressed at a greater level than a level of suppression observed for a similar nucleic acid sequence. In some embodiments, suppression of the transcripts of the target nucleic acid sequence can be 1.1-100, 2-100, 1.5, 2, 2.5, 3, 4, 5, 6, 7, 8, 9, or 10-fold greater than suppression observed for a similar nucleic acid sequence. In some embodiments, a target nucleic acid sequence is associated with (or more associated with compared to a similar nucleic acid sequence) a condition, disorder or disease. As those skilled in the art will appreciate, selective reduction of a transcript (and/or products thereof) associated with conditions, disorders or diseases, while maintaining transcripts that are not, or are less, associated with conditions, disorders or diseases can provide a number of advantages, for example, providing disease treatment and/or prevention while maintaining one or more desired biological functions (which may provide, among other things, fewer or less severe side effects).

In some embodiments, selectivity is at least 10 fold, or 20, 30, 40, or 50 fold or more in a system, e.g. a reporter assay described herein. In some embodiments, an oligonucleotide or composition can effectively reduce levels of mutant protein (e.g., at least 50%, 60%, 70% or more reduction of a mutant protein) while maintaining levels of wild-type protein (e.g. at least 70%, 75%, 80%, 85%, 90%, 95%, or more wild-type protein remaining) in a system. In some embodiments, provided oligonucleotides are stable in various biological systems, e.g. in mouse brain homogenates (e.g., at least 70%, 75%, 80%, 85%, 90%, 95%, or more remaining after 1, 2, 3, 4, 5, 6, 7, or 8 days). In some embodiments, provided oligonucleotides are of low toxicity. In some embodiments, provided oligonucleotides and compositions thereof, e.g., chirally controlled oligonucleotides and compositions thereof, do not significant activate TLR9 (e.g., when compared to reference oligonucleotides and compositions thereof (e.g., corresponding stereorandom oligonucleotides and compositions thereof)). In some embodiments, provided oligonucleotides and compositions thereof, e.g., chirally controlled oligonucleotides and compositions thereof, do not significantly induce complement activation (e.g., when compared to reference oligonucleotides and compositions thereof (e.g., corresponding stereorandom oligonucleotides and compositions thereof)).

For various applications, provided oligonucleotides and/or compositions may be provided as pharmaceutical compositions. In some embodiments, the present disclosure provides a pharmaceutical composition which comprises or delivers an effective amount of an oligonucleotide or a pharmaceutically acceptable salt thereof. In some embodiments, a pharmaceutical composition may comprise various forms of an oligonucleotide, e.g., acid, base and various pharmaceutically acceptable salt forms. In some embodiments, a pharmaceutically acceptable salt is sodium salt. In some embodiments, a pharmaceutically acceptable salt is a potassium salt. In some embodiments, a pharmaceutically acceptable salt is a amine salt (e.g., of an amine having the structure of N(R)₃). In some embodiments, a pharmaceutical composition further comprises a pharmaceutically acceptable carrier. In some embodiments, a pharmaceutical composition is or comprises a liquid solution. In some embodiments, a liquid composition has a controlled pH range, e.g., around or being physiological pH. In some embodiments, a pharmaceutical composition comprises or is formulated as a solution in a physiologically compatible buffers such as Hanks's solution, Ringer's solution, cerebral spinal fluid, artificial cerebral spinal fluid (aCSF) or physiological saline buffer. In some embodiments, a pharmaceutical composition comprises or is formulated as a solution in artificial cerebral spinal fluid (aCSF). In some embodiments, a pharmaceutical composition is an injectable suspension or solution. In certain embodiments, injectable suspensions or solutions are prepared using appropriate liquid carriers, suspending agents and the like. Pharmaceutical compositions can be administered in various suitable routes. In some embodiments, pharmaceutical compositions are formulated for oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, intrathecal, intracerebroventricular or epidural injection as, for example, a sterile solution or suspension, e.g., in physiologically compatible buffers such as Hanks's solution, Ringer's solution, artificial cerebral spinal fluid (aCSF) or physiological saline buffer or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Among other things, the present disclosure provides technologies for preventing or treating conditions, disorders or diseases. In some embodiments, the present disclosure provides a method for preventing or treating a condition, disorder or disease, comprising administering or delivering to a subject susceptible thereto or suffering therefrom an effective amount of an oligonucleotide or composition as described herein. In some embodiments, a condition, disorder or disease is amenable to (e.g., can benefit from) A to I conversion. In some embodiments, the present disclosure provides a method for preventing or treating a condition, disorder or disease associated with a G to A mutation, comprising administering to a subject susceptible thereto or suffering therefrom an effective amount of an oligonucleotide or composition as described herein. In some embodiments, the present disclosure provides a method for preventing or treating a condition, disorder or disease amenable to a G to A mutation, comprising administering to a subject susceptible thereto or suffering therefrom an effective amount of an oligonucleotide or composition as described herein. In some embodiments, the present disclosure provides a method for preventing or treating a condition, disorder or disease associated with a G to A mutation, comprising administering to a subject susceptible thereto or suffering therefrom an effective amount of an oligonucleotide or composition as described herein. In some embodiments, the base sequence of the oligonucleotide or oligonucleotides in the oligonucleotide composition is substantially complementary to that of the target nucleic acid comprising a target adenosine. In some embodiments, cells, tissues or organs associated with the condition, disorder or disease comprise or express an ADAR protein. In some embodiments, cells, tissues or organs associated with the condition, disorder or disease comprise or express ADAR1 (e.g., a p110 and/or a p150 forms). In some embodiments, cells, tissues or organs associated with the condition, disorder or disease comprise or express ADAR2. In some embodiments, a condition, disorder or disease is as described herein. In some embodiments, a condition, disorder or disease is alpha-1 antitrypsin deficiency. In some embodiments, a method comprises converting a target adenosine to I.

In some embodiments, the present disclosure provides an oligonucleotide comprising a sequence complementary to a target sequence. In some embodiments, the present disclosure provides an oligonucleotide which directs site-specific (can also be referred as site directed) editing (e.g., deamination). In some embodiments, the present disclosure provides an oligonucleotide which directs site-specific adenosine editing mediated by ADAR (e.g., an endogenous ADAR). Various provided oligonucleotides can be utilized as single-stranded oligonucleotides for site-directed editing of a nucleotide in a target RNA sequence. In some embodiments, the present disclosure provides methods for preventing and/or treating conditions, disorders, or diseases associated with a G to A mutation in a target sequence using provided single-stranded oligonucleotides for site-directed editing of a nucleotide in a target RNA sequence and compositions thereof. In some embodiments, the present disclosure provides oligonucleotides and compositions thereof for use as medicaments, e.g., for conditions, disorders, or diseases associated with a G to A mutation in a target sequence. In some embodiments, the present disclosure provides oligonucleotides and compositions thereof for use in the treatment of conditions, disorders or diseases associated with a G to A mutation in a target sequence. In some embodiments, the present disclosure provides oligonucleotides and compositions thereof for the manufacture of medicaments for the treatment of a related conditions, disorders or diseases associated with a G to A mutation in a target sequence.

In some embodiments, the present disclosure provides a method for preventing, treating or ameliorating a condition, disorder or disease associated with a G to A mutation in a target sequence in a subject susceptible thereto or suffering therefrom, comprising administering to the subject a therapeutically effective amount of an oligonucleotide or a pharmaceutical composition thereof.

In some embodiments, the present disclosure provides a method for deaminating a target adenosine in a target sequence in a cell, comprising: contacting the cell with an oligonucleotide or a composition thereof. In some embodiments, the present disclosure provides a method deaminating a target adenosine in a target sequence (e.g., a transcript) in a cell, comprising: contacting the cell with an oligonucleotide or a composition thereof. In some embodiments, the present disclosure provides a method for reducing the level of a protein associated with a G to A mutation in a cell, comprising: contacting the cell with an oligonucleotide or a composition thereof. In some embodiments, provided methods can selectively reduce levels of a transcripts and/or products encoded thereby that are related to conditions, disorders or diseases associated with a G to A mutation. In some embodiments, provided methods can selectively edit target nucleic acids, e.g., transcripts comprising an undesired A (e.g., a G to A mutation) over otherwise identical nucleic acids which have G at positions of target A.

In some embodiments, the present disclosure provides a method for decreasing a mutated gene (e.g., a G to A mutation) expression in a mammal in need thereof, comprising administering to the mammal a nucleic acid-lipid particle comprising a provided single-stranded oligonucleotide for site-directed editing of a nucleotide in a target RNA sequence or a composition thereof.

In some embodiments, the present disclosure provides a method for in vivo delivery of an oligonucleotide, comprising administering to a mammal an oligonucleotide or a composition thereof.

In some embodiments, a subject or patient suitable for treatment of a condition, disorder, or disease associated with a G to A mutation, can be identified or diagnosed by a health care professional.

In some embodiments, a symptom of a condition, disorder or disease associated with a G to A mutation can be any condition, disorder or disease that can benefit from an A to I conversion.

In some embodiments, a provided single-stranded oligonucleotide for site-directed editing of a nucleotide in a target RNA sequence or a composition thereof can prevent, treat, ameliorate, or slow progression of a condition, disorder or disease associated with a G to A mutation, or at least one symptom of a condition, disorder or disease associated with a G to A mutation.

In some embodiments, a method of the present disclosure can be for the treatment of a condition, disorder or disease associated with a G to A mutation in a subject wherein the method comprises administering to a subject a therapeutically effective amount of an oligonucleotide or a pharmaceutical composition thereof.

In some embodiments, a provided method can reduce at least one symptom of a condition, disorder or disease associated with a G to A mutation wherein the method comprises administering to a subject a therapeutically effective amount of an oligonucleotide or a pharmaceutical composition thereof.

In some embodiments, administration of an oligonucleotide to a patient or subject can be capable of mediating any one or more of: slowing the progression of a condition, disorder or disease associated with a G to A mutation; delaying the onset of a condition, disorder or disease associated with a G to A mutation or at least one symptom thereof; improving one or more indicators of a condition, disorder or disease associated with a G to A mutation; and/or increasing the survival time or lifespan of the patient or subject.

In some embodiments, slowing disease progression can relate to the prevention of, or delay in, a clinically undesirable change in one or more clinical parameters in an individual susceptible to or suffering from a condition, disorder, or disease associated with a G to A mutation, such as those described herein. It is well within the abilities of a physician to identify a slowing of disease progression in an individual susceptible to or suffering a condition, disorder, or disease associated with a G to A mutation, using one or more of the disease assessment tests described herein. Additionally, it is understood that a physician may administer to the individual diagnostic tests other than those described herein to assess the rate of disease progression in an individual susceptible to or suffering from a condition, disorder, or disease associated with a G to A mutation.

A physician may use family history of a condition, disorder, or disease associated with a G to A mutation or comparisons to other patients with similar genetic profile.

In some embodiments, indicators of a condition, disorder, or disease associated with a G to A mutation include parameters employed by a medical professional, such as a physician, to diagnose or measure the progression of the condition, disorder, or disease.

In some embodiments, a subject is administered an oligonucleotide or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components can be administered as one composition. In some embodiments, at a time point a subject being administered can be exposed to both provided oligonucleotides and additional components at the same time.

In some embodiments, an additional therapeutic agent can be physically conjugated to an oligonucleotide. In some embodiments, an additional agent is GalNAc. In some embodiments, a provided single-stranded oligonucleotide for site-directed editing of a nucleotide in a target RNA sequence can be physically conjugated with an additional agent. In some embodiments, additional agent oligonucleotides can have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure, wherein each T may be independently replaced with U and vice versa. In some embodiments, an oligonucleotide can be physically conjugated to a second oligonucleotide which can decrease (directly or indirectly) the expression, activity, and/or level of a target sequence, or which is useful for treating a condition, disorder, or disease associated with a G to A mutation.

In some embodiments, a provided single-stranded oligonucleotide for site-directed editing of a nucleotide in a target RNA sequence may be administered with one or more additional (or second) therapeutic agent for a condition, disorder or disease associated with a G to A mutation.

In some embodiments, a subject can be administered an oligonucleotide and an additional therapeutic agent, wherein the additional therapeutic agent is an agent described herein or known in the art which is useful for treatment of a condition, disorder or disease to be treated.

In some embodiments, provided single-stranded oligonucleotide for site-directed editing of a nucleotide in a target RNA sequence can be co-administered or be used as part of a treatment regimen along with one or more treatment for a condition, disorder or disease or a symptom thereof, including but not limited to: aptamers, lncRNAs, lncRNA inhibitors, antibodies, peptides, small molecules, other oligonucleotides to a target other targets.

In some embodiments, an additional therapeutic treatment is, as a non-limiting example, a method of editing a gene

In some embodiments, an additional therapeutic agent is, as a non-limiting example, an oligonucleotide.

In some embodiments, a second or additional therapeutic agent can be administered to a subject prior, simultaneously with, or after an oligonucleotide. In some embodiments, a second or additional therapeutic agent can be administered multiple times to a subject, and an oligonucleotide is also administered multiple times to a subject, and the administrations are in any order.

In some embodiments, an improvement may include decreasing the expression, activity and/or level of a gene or gene product which is too high in a disease state; increasing the expression, activity and/or level of a gene or gene product which is too low in the disease state; and/or decreasing the expression, activity and/or level of a mutant and/or disease-associated variant of a gene or gene product.

In some embodiments, an oligonucleotide or composition useful for treating, ameliorating and/or preventing a condition, disorder or disease associated with a G to A mutation can be administered (e.g., to a subject) via various suitable available technologies.

In some embodiments, provided oligonucleotides, e.g., single-stranded oligonucleotide for site-directed editing of a nucleotide in a target RNA sequences, can be administered as a pharmaceutical composition, e.g., for treating, ameliorating and/or preventing conditions, disorders or diseases. In some embodiments, provided oligonucleotides comprise at least one chirally controlled internucleotidic linkage. In some embodiments, provided oligonucleotide compositions are chirally controlled.

Among other things, technologies, e.g., oligonucleotides and compositions thereof, of the present disclosure can provide various improvements and advantages compared to reference technologies (e.g., absence or low levels of chiral control (e.g., stereorandom oligonucleotide compositions (e.g., of oligonucleotides of the same base sequence, or the same constitution, etc.)), and/or absence or low levels of certain modifications and patterns thereof (e.g., 2′-F, non-negatively charged internucleotidic linkages, etc.), such as improved stability, delivery, editing efficiency, pharmacokinetics, and/or pharmacodynamics. In some embodiments, a reference oligonucleotide composition is a stereorandom oligonucleotide composition of oligonucleotides with the same base sequence. In some embodiments, a reference oligonucleotide composition is a stereorandom oligonucleotide composition of oligonucleotides with the same constitution (as appreciated by those skilled in the art, in some embodiments, various salt forms may be properly considered to be of the same constitution). In some embodiments, a reference oligonucleotide is an oligonucleotide comprising no non-negatively charged internucleotidic linkages. In some embodiments, a reference oligonucleotide comprises no n001. In some embodiments, a reference oligonucleotide composition is a composition of oligonucleotides comprising no non-negatively charged internucleotidic linkages. In some embodiments, a reference oligonucleotide composition is a composition of oligonucleotides comprising no n001. In some embodiments, provided technologies may be utilized at lower unit or total doses, and/or may be administered with fewer doses and/or longer dose intervals (e.g., to achieve comparable or better effects) compared to reference technologies. In some embodiments, provided technologies can provide long durability of editing. In some embodiments, provided technologies once administered can provide activities, e.g., target editing, at or above certain levels (e.g., levels useful and/or sufficient to provide certain biological and/or therapeutic effects) for a period of time, e.g., about or at least about 2, 3, 4, 5, 6, 7, 8, 9, 10, 15, 20, 25, 30, 35, 40, 45, 50, 60 or more days, or 1, 2, 3, 4, 5, 6, 7, 8, 9, or 10 months, after a last dose. In some embodiments, provided technologies provide low toxicity. In some embodiments, provided technologies may be utilized at higher unit or total doses, and/or may be administered with more doses and/or shorter dose intervals (e.g., to achieve better effects) compared to reference technologies. In some embodiments, a total dose may be administered as a single dose. In some embodiments, a total dose may be administered as two or more single doses. In some embodiments, a total dose administered as a single dose may provide higher maximum editing levels compared to when administered as two or more single doses.

In some cases, patients who have been administered an oligonucleotide as a medicament may experience certain side effects or adverse effects, including: thrombocytopenia, renal toxicity, glomerulonephritis, and/or coagulation abnormalities; genotoxicity, repeat-dose toxicity of target organs and pathologic effects; dose response and exposure relationships; chronic toxicity; juvenile toxicity; reproductive and developmental toxicity; cardiovascular safety; injection site reactions; cytokine response complement effects; immunogenicity; and/or carcinogenicity. In some embodiments, an additional therapeutic agent is administered to counter-act a side effect or adverse effect of administration of an oligonucleotide. In some embodiments, a particular single-stranded oligonucleotide for site-directed editing of a nucleotide in a target RNA sequence can have a reduced capability of eliciting a side effect or adverse effect, compared to a different single-stranded oligonucleotide for site-directed editing of a nucleotide in a target RNA sequence.

In some embodiments, an additional therapeutic agent can be administered to the patient in order to control or alleviate one or more side effects or adverse effects associated with administration of an oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide, and wherein the oligonucleotide operates via any biochemical mechanism, including but not limited to: decreasing the level, expression and/or activity of a target gene or a gene product thereof, increasing or decreasing skipping of one or more exons in a target gene mRNA, an ADAR-mediated deamination, a RNaseH-mediated mechanism, a steric hindrance-mediated mechanism, and/or a RNA interference-mediated mechanism, wherein the oligonucleotide is single- or double-stranded.

In some embodiments, an oligonucleotide composition and one or more additional therapeutic agent can be administered to a patient (in any order), wherein the additional therapeutic agent can be administered to the patient in order to control or alleviate one or more side effects or adverse effects associated with administration of the oligonucleotide composition, and wherein the oligonucleotide composition can be chirally controlled or comprises at least one chirally controlled internucleotidic linkage (including but not limited to a chirally controlled phosphorothioate).

Various conditions, disorders, or diseases can benefit from adenosine editing, including those are associated with a G to A mutation, e.g., Cystic fibrosis, Hurler Syndrome, alpha-I -antitrypsin (A1AT) deficiency, Parkinson's disease, Alzheimer's disease, albinism, Amyotrophic lateral sclerosis, Asthma, 3-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and various cancers. Certain conditions, disorders or diseases are described in WO 2016/097212, WO 2017/220751, WO 2018/041973, WO 2018/134301A1, WO 2020/154342, WO 2020/154343, WO 2020/154344, WO 2020/165077, WO 2020/201406, WO 2020/216637, WO 2020/252376, WO 2021/130313, WO 2021/231691, WO 2021/231692, WO 2021/231673, WO 2021/231675, WO 2021/231679, WO 2021/231680, WO 2021/231698, WO 2021/231685, WO 2021/231830, WO 2021/243023, WO 2022/018207, WO 2022/026928, or WO 2022/090256.

In some embodiments, a condition, disorder or disease is Alpha-1 antitrypsin (A1AT) deficiency (AATD).

Alpha-1 antitrypsin (A1AT) deficiency (AATD) is a genetic disease reportedly caused by defects in the SERPINA1 gene (also known as PI; AIA; AAT; PII; A1AT; PR02275; and alpha1AT). Severe AlAT deficiency is associated with various phenotypes including lung and liver phenotypes.

A1AT deficiency is reportedly one of the most common genetic diseases in subjects of Northern European descent. Prevalence of severe A1AT deficiency in the U.S. alone is 80,000-100,000. Similar numbers are estimated to be found in the EU. The worldwide estimate for severe A1AT deficiency has been pegged at 3 million people. A1AT deficiency causes emphysema, with subjects developing emphysema in their third or fourth decade. A1AT deficiency can also cause liver failure and hepatocellular carcinoma, with up to 30% of subjects with severe A1AT deficiency developing significant liver disease, including cirrhosis, fulminant liver failure, and hepatocellular carcinoma.

A mutation (i.e., c. 1024G>A) in SERPINA1 gene leads to a glutamate to lysine substitution at amino acid position 342 (E342K, “Z mutation”) of the mature A1AT protein. This missense mutation affect protein conformation and secretion leading to reduced circulating levels of A1AT. Alleles carrying the Z mutation are identified as PiZ alleles. Subjects homozygous for the PiZ allele are termed PiZZ carriers, and express 10-15% of normal levels of serum A1AT. Approximately 95% of subjects who are symptomatic for AAT deficiency have the PiZZ genotype. Subjects heterozygous for the Z mutation are termed PiMZ mutants, and express 60% of normal levels of serum A1AT. Of those diagnosed, 90% of patients with severe A1AT deficiency have the ZZ mutation. About between 30,000 and 50,000 individuals in the United States have the PiZZ genotype.

The pathophysiology of AAT deficiency can vary by the organ affected. Liver disease is reported to be due to a gain-of-function mechanism. Abnormally folded A1AT, especially Z-type A1AT (Z-AT), aggregates and polymerizes within hepatocytes. A1AT inclusions are found in PiZZ subjects and are thought to cause cirrhosis and, in some cases, hepatocellular carcinoma. Evidence for the gain-of-function mechanism in liver disease is supported by null homozygotes. These subjects produce no A1AT and do not develop hepatocyte inclusions or liver disease.

It is reported that A1AT deficiency leads to liver disease in up to about 50% of A1AT subjects and leads to severe liver disease in up to about 30% of subjects. Liver disease may manifest as: (a) cirrhosis during childhood that is self-limiting, (b) severe cirrhosis during childhood or adulthood that requires liver transplantation or leads to death and (c) hepatocellular carcinoma that is often deadly. The onset of liver disease is reported to be bi-modal, predominantly affecting children or adults. Childhood disease is self-limiting in many cases but may be led to end-stage, deadly cirrhosis. It is reported that up to about 18% of subjects with the PiZZ genotype may develop clinically significant liver abnormalities during childhood. Approximately 2% of PiZZ subjects are reported to develop severe liver cirrhosis leading to death during childhood (Sveger 1988; Volpert 2000). Adult-onset liver disease may affect subjects with all genotypes, but presents earlier in subjects with the PiZZ genotype. Approximately 2-10% of A1AT deficient subjects are reported to develop adult-onset liver disease.

Lung disease associated with A 1AT deficiency is currently treated with intravenous administration of human-derived replacement A1AT protein, but in addition to being costly and requiring frequent injections over a subject's entire lifetime, this approach is only partially effective. A1AT-deficient subjects with hepatocellular carcinoma are currently treated with chemotherapy and surgery, but there is no satisfactory approach for preventing the potentially deadly liver manifestations of A1AT deficiency.

Among other things, the present disclosure recognizes a need for improved treatment of A1AT deficiency, e.g., including liver and lung manifestations thereof. In some embodiments, the present disclosure provides technologies for preventing or treating conditions, disorders or diseases associated Alpha-1 antitrypsin (A1AT) deficiency, e.g., by providing oligonucleotides and/or compositions that can convert the A mutation to I which can be read as G during protein translation and thus correcting the G to A mutation for protein translation. Among other things, alteration of SERPINA1 in one or more of hepatocytes can prevent the progression of liver disease in subjects with A1AT deficiency by reducing or eliminating production of the toxic Z protein (Z-AAT). In certain embodiments, Z protein production is eliminated or reduced by utilizing provided technologies. In certain embodiments, the disease is cured, does not progress, or has delayed progression compared to a subject who has not received the therapy.

In some embodiments, AATD dual pathologies have been reported in liver and lung. In some embodiments, inability to secrete polymerized Z-ATT has been reported to lead to, e.g., liver damage/cirrhosis. In some embodiments, one or both lungs are open to unchecked proteases, which in some embodiments lead to inflammation and lung damage. Many patients (e.g., reportedly ˜200,000 in the US and EU) are with homozygous ZZ genotype which is reported to be associated with the most common form of sever AATD. It has been reported that approved therapies modestly increase circulating levels of wide-type AAT in those with lung pathology, and no therapies address liver pathology. In some embodiments, provided technologies increase or restore expression, levels, properties and/or activities of wild-type AAT in liver. In some embodiments, provided technologies target liver, e.g., through incorporating moieties targeting liver (e.g., ligands such as GalNAc targeting receptors expressed in liver) into oligonucleotides. In some embodiments, provided technologies restore, increase or enhance wild-type AAT physiological regulation in liver. In some embodiments, provided technologies reduce Z-AAT protein aggregation. In some embodiments, provided technologies restore, increase or enhance wild-type AAT physiological regulation in liver and reduce Z-AAT protein aggregation. In some embodiments, provided technologies increase secretion into bloodstream. In some embodiments, provided technologies increase circulating wild-type AAT. In some embodiments, provided technologies increase circulating, lung-bond wild-type AAT. In some embodiments, provided technologies increase or restore expression, levels, properties and/or activities of wild-type AAT in lung. In some embodiments, provided technologies protect lungs from undesired proteases. In some embodiments, provided technologies reduce or prevent inflammation and/or lung damage. In some embodiments, provided technologies provide benefits at both livers and lungs. In some embodiments, provided technology reduces or prevents liver damage or cirrhosis, and reduces or prevents inflammation and/or lung damage. In some embodiments, provided oligonucleotides, e.g., those comprising certain moieties such ligands (e.g., GalNAc) targeting receptors expressed in livers, provide benefits at livers and lungs. In some embodiments, provided technologies simultaneously provide benefits at livers and lungs. In some embodiments, provided technologies address lung and/or liver manifestation of AATD. In some embodiments, provided technologies simultaneously address lung and liver manifestation of AATD. In some embodiments, provided technologies comprise using GalNAc conjugated oligonucleotides and compositions thereof to correct RNA base mutation in mRNA coded by SERPINA1 Z allele that triggers AATD. In some embodiments, provided technologies simultaneously reduce aggregation of mutated, misfolded alpha-1 protein and increase circulating levels of wild-type alpha-1 antitrypsin protein, and in some embodiments address both liver and lung manifestations of AATD. In some embodiments, provided technologies avoid risk of permanent off-target changes to DNA.

In certain embodiments, technologies as described herein can provide a selective advantage to survival of one or more of treated hepatocytes. In certain embodiments, a target cell is modified. In some embodiments, cells treated with technologies herein may not produce toxic Z protein. In some embodiments, diseased cells that are not modified produce toxic Z proteins and may undergo apoptosis secondary to endoplasmic reticulum (ER) stress induced by Z protein misfolding. In certain embodiments, after treatment using the provided technologies, treated cells will survive and untreated cells will die. This selective advantage can drive eventual colonization of hepatocytes with the majority being SERPINA1 corrected cells.

In some embodiments, an oligonucleotide, when administered to a patient suffering from or susceptible to a condition, disorder or disease that is associated with a G to A mutation is capable of reducing at least one symptom of the condition, disorder or disease and/or capable of delaying or preventing the onset, worsening, and/or reducing the rate and/or degree of worsening of at least one symptom of the condition, disorder or disease that's due to a G to A mutation in a gene or gene product.

In some embodiments, provided technologies can provide editing of two or more sites in a system (e.g., a cell, tissue, organ, animal, etc.) (“multiplex editing”). In some embodiments, provided technologies can target and provide editing of two or more sites of the same transcripts. In some embodiments, provided technologies can target and provide editing of two or more different transcripts, either from the same nucleic acid or different nucleic acids. In some embodiments, provided technologies can target and provide editing of transcripts from two or more different nucleic acids. In some embodiments, provided technologies can target and provide editing of transcripts from two or more different genes. In some embodiments, of the targets simultaneously edited, each is independently at a biologically and/or therapeutically relevant level. In some embodiments, in multiplex editing one or more or all targets are independently edited at a comparable level as editing conducted individually under comparable conditions. In some embodiments, multiplex editing are performed utilizing two or more separate compositions, each of which independently target one or more targets. In some embodiments, compositions are administered concurrently. In some embodiments, compositions are administered with suitable intervals. In some embodiments, one or more compositions are administered prior or subsequently to one or more other compositions. In some embodiments, multiplex editing are performed utilizing a single composition, e.g., a composition comprising two or more pluralities of oligonucleotides, wherein the pluralities target different targets. In some embodiments, each plurality independently targets a different adenosine. In some embodiments, each plurality independently targets a different transcript. In some embodiments, each plurality independently targets a different gene. In some embodiments, two or more pluralities may target the same target, but the pluralities together target the desired targets.

As described herein, provided technologies can provide a number of advantages. For example, in some embodiments, provided technologies are safer than technologies that act on DNA, as provided technologies can provide RNA edits that are both reversible and tunable (e.g., through adjusting of doses). Additionally and alternatively, provided technologies can provide high levels of editing in systems expressing endogenous ADAR proteins thus avoiding the requirement of introduction of exogenous proteins in various instances. Still further, provided technologies do not require complex oligonucleotides that depend on ancillary delivery vehicles, such as viral vectors or lipid nanoparticles, as utilized in many other technologies, particularly for application beyond cell culture. In some embodiments, provided technologies can provide sequence-specific A-to-I RNA editing with high efficiency using endogenous ADAR enzymes and can be delivered to various systems, e.g., cells, in the absence of artificial delivery agents.

Those skilled in the art reading the present disclosure will understand that provided oligonucleotides and compositions thereof may be delivered using a number of technologies in accordance with the present disclosure. In some embodiments, provided oligonucleotides and compositions may be delivered via transfection or lipofeciton. In some embodiments, provided oligonucleotides and compositions thereof may be delivered in the absence of delivery aids, such as those utilized in transfection or lipofection. In some embodiments, provided oligonucleotides and compositions may be delivered via transfection or lipofeciton. In some embodiments, provided oligonucleotides and compositions thereof are delivered with gymnotic delivery. In some embodiments, provided oligonucleotides comprise additional chemical moieties that can facilitate delivery. For example, in some embodiments, additional chemical moieties are or comprise ligand moieties (e.g., N-acetylgalactosamine (GalNAc)) for receptors (e.g., asialoglycoprotein receptors). In some embodiments, provided oligonucleotides and compositions thereof can be delivered through GalNAc-mediated delivery.

Among other things, the present disclosure provides the following Example Embodiments:

1. An oligonucleotide comprising:
a first domain; and
a second domain,
   wherein:
the base sequence of the first domain is complementary to a first portion of the base sequence of a target nucleic acid;
the base sequence of the second domain is complementary to a second portion of the base sequence of a target nucleic acid; and
the first portion and the second portion of the base sequence of the target nucleic acid are separated by a gap.
   2. An oligonucleotide comprising:
a first domain; and
a second domain,
   wherein:
the first domain is characterized in that it can form a duplex with a first portion of a target nucleic acid;
the second domain is characterized in that it can form a duplex with a second portion of a target nucleic acid; and
the first portion and the second portion of the base sequence of the target nucleic acid are separated by a gap.
   3. The oligonucleotide of any one of the preceding Embodiments, wherein the length of the first domain is about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases.
   4. The oligonucleotide of any one of the preceding Embodiments, wherein about or at least about 5-100, 5-50, 5-40, 5-30, 5-20, 10-100, 10-50, 10-40, 10-30, 10-20,5,6,7,8,9, 10, 11, 12, 13, 14, 15, 16, 17, 18, 19, 20, 21, 22, 23, 24, 25, 26, 27, 28, 29 or 30 nucleobases in the first domain are each independently optionally substituted A, T, C, G, or U, or an optionally substituted tautomer of A, T, C, G or U.
   5. The oligonucleotide of any one of the preceding Embodiments, wherein the length of the first domain is about 10-30 nucleobases.
   6. The oligonucleotide of any one of the preceding Embodiments, wherein about 10-30 nucleobases in the first domain are each independently optionally substituted A, T, C, G, or U, or an optionally substituted tautomer of A, T, C, G or U.
   7. The oligonucleotide of any one of the preceding Embodiments, wherein the length of the first domain is about 10-25 nucleobases.
   8. The oligonucleotide of any one of the preceding Embodiments, wherein about 10-25 nucleobases in the first domain are each independently optionally substituted A, T, C, G, or U, or an optionally substituted tautomer of A, T, C, G or U.
   9. The oligonucleotide of any one of the preceding Embodiments, wherein the length of the first domain is about 21 or more nucleobases.
   10. The oligonucleotide of any one Embodiments 1-8, wherein the length of the first domain is about 20 nucleobases.
   11. The oligonucleotide of any one Embodiments 1-8, wherein the length of the first domain is about 19 nucleobases.
   12. The oligonucleotide of any one Embodiments 1-8, wherein the length of the first domain is about 18 nucleobases.
   13. The oligonucleotide of any one Embodiments 1-8, wherein the length of the first domain is about 17 nucleobases.
   14. The oligonucleotide of any one Embodiments 1-8, wherein the length of the first domain is about 16 nucleobases.
   15. The oligonucleotide of any one Embodiments 1-8, wherein the length of the first domain is about 15 nucleobases.
   16. The oligonucleotide of any one Embodiments 1-8, wherein the length of the first domain is about 14 nucleobases.
   17. The oligonucleotide of any one Embodiments 1-8, wherein the length of the first domain is about 13 nucleobases.
   18. The oligonucleotide of any one Embodiments 1-8, wherein the length of the first domain is about 12 nucleobases.
   19. The oligonucleotide of any one Embodiments 1-8, wherein the length of the first domain is about 11 nucleobases.
   20. The oligonucleotide of any one Embodiments 1-8, wherein the length of the first domain is about 10 nucleobases.
   21. The oligonucleotide of any one of the preceding Embodiments, wherein about 10 nucleobases in the first domain are each independently optionally substituted A, T, C, G, or U, or an optionally substituted tautomer of A, T, C, G or U.
   22. The oligonucleotide of any one Embodiments 1-19, wherein about 11 nucleobases in the first domain are each independently optionally substituted A, T, C, G, or U, or an optionally substituted tautomer of A, T, C, G or U.
   23. The oligonucleotide of any one Embodiments 1-18, wherein about 12 nucleobases in the first domain are each independently optionally substituted A, T, C, G, or U, or an optionally substituted tautomer of A, T, C, G or U.
   24. The oligonucleotide of any one Embodiments 1-17, wherein about 13 nucleobases in the first domain are each independently optionally substituted A, T, C, G, or U, or an optionally substituted tautomer of A, T, C, G or U.
   25. The oligonucleotide of any one Embodiments 1-16, wherein about 14 nucleobases in the first domain are each independently optionally substituted A, T, C, G, or U, or an optionally substituted tautomer of A, T, C, G or U.
   26. The oligonucleotide of any one Embodiments 1-15, wherein about 15 nucleobases in the first domain are each independently optionally substituted A, T, C, G, or U, or an optionally substituted tautomer of A, T, C, G or U.
   27. The oligonucleotide of any one Embodiments 1-14, wherein about 16 nucleobases in the first domain are each independently optionally substituted A, T, C, G, or U, or an optionally substituted tautomer of A, T, C, G or U.
   28. The oligonucleotide of any one Embodiments 1-13, wherein about 17 nucleobases in the first domain are each independently optionally substituted A, T, C, G, or U, or an optionally substituted tautomer of A, T, C, G or U.
   29. The oligonucleotide of any one Embodiments 1-12, wherein about 18 nucleobases in the first domain are each independently optionally substituted A, T, C, G, or U, or an optionally substituted tautomer of A, T, C, G or U.
   30. The oligonucleotide of any one Embodiments 1-11, wherein about 19 nucleobases in the first domain are each independently optionally substituted A, T, C, G, or U, or an optionally substituted tautomer of A, T, C, G or U.
   31. The oligonucleotide of any one Embodiments 1-10, wherein about 20 nucleobases in the first domain are each independently optionally substituted A, T, C, G, or U, or an optionally substituted tautomer of A, T, C, G or U.
   32. The oligonucleotide of any one Embodiments 1-9, wherein about 21 or more nucleobases in the first domain are each independently optionally substituted A, T, C, G, or U, or an optionally substituted tautomer of A, T, C, G or U.
   33. The oligonucleotide of any one of the preceding Embodiments, wherein the first domain and the first portion have the same lengths.
   34. The oligonucleotide of any one of the preceding Embodiments, wherein the base sequence of the first domain is about 80%-100% complementary to the base sequence of the first portion.
   35. The oligonucleotide of any one of the preceding Embodiments, wherein the base sequence of the first domain comprises a mismatch with the base sequence of the first portion.
   36. The oligonucleotide of any one of the preceding Embodiments, wherein the base sequence of the first domain comprises a wobble with the base sequence of the first portion.
   37. The oligonucleotide of any one of the preceding Embodiments, wherein the base sequence of the first domain comprises a bulge with the base sequence of the first portion.
   38. The oligonucleotide of any one of the preceding Embodiments, wherein the base sequence of the first domain is about or at least about 85% complementary to the base sequence of the first portion.
   39. The oligonucleotide of any one of the preceding Embodiments, wherein the base sequence of the first domain is about or at least about 90% complementary to the base sequence of the first portion.
   40. The oligonucleotide of any one of the preceding Embodiments, wherein the base sequence of the first domain is about or at least about 95% complementary to the base sequence of the first portion.
   41. The oligonucleotide of any one of the preceding Embodiments, wherein the first nucleobase from the 5′-end of the first domain is complementary to a nucleobase in the first portion.
   42. The oligonucleotide of any one of the preceding Embodiments, wherein the first nucleobase from the 3′-end of the first domain is complementary to a nucleobase in the first portion.
   43. The oligonucleotide of any one of Embodiments 1-34, wherein the base sequence of the first domain is 100% complementary to the base sequence of the first portion.
   44. The oligonucleotide of any one of the preceding Embodiments, wherein when the oligonucleotide is administered or delivered to a system comprising a target nucleic acid or a fragment thereof that comprises the first portion, the first domain can selectively form a duplex with the first portion.
   45. The oligonucleotide of any one of the preceding Embodiments, wherein when the oligonucleotide is administered or delivered to a system expressing a target nucleic acid or a fragment thereof that comprises the first portion, the first domain can selectively form a duplex with the first portion.
   46. The oligonucleotide of any one of the preceding Embodiments, wherein the base sequence of the first portion differentiates the first portion from the rest of the target nucleic acid.
   47. The oligonucleotide of any one of the preceding Embodiments, wherein the base sequence of the first portion differentiates the first portion from the other transcripts in a system.
   48. The oligonucleotide of any one of the preceding Embodiments, wherein the base sequence of the first portion differentiates the first portion from the other RNA in a system.
   49. The oligonucleotide of any one of the preceding Embodiments, wherein the base sequence of the first portion differentiates the first portion from the other nucleic acids in a system.
   50. The oligonucleotide of any one of the preceding Embodiments, wherein the length of the second domain is about or at least about 5-100, 5-50, 5-40, 5-30, 5-20, 10-100, 10-50, 10-40, 10-30, 10-20, 5, 6, 7, 8, 9, 10, 11, 12, 13, 14, 15, 16, 17, 18, 19, 20, 21, 22, 23, 24, 25, 26, 27, 28, 29 or 30 nucleobases.
   51. The oligonucleotide of any one of the preceding Embodiments, wherein about or at least about 5-100, 5-50, 5-40, 5-30, 5-20, 10-100, 10-50, 10-40, 10-30, 10-20,5, 6,7, 8, 9, 10, 11, 12, 13, 14, 15, 16, 17, 18, 19, 20, 21, 22, 23, 24, 25, 26, 27, 28, 29 or 30 nucleobases in the second domain are each independently optionally substituted A, T, C, G, or U, or an optionally substituted tautomer of A, T, C, G or U.
   52. The oligonucleotide of any one of the preceding Embodiments, wherein the length of the second domain is about 10-30 nucleobases.
   53. The oligonucleotide of any one of the preceding Embodiments, wherein about 10-30 nucleobases in the second domain are each independently optionally substituted A, T, C, G, or U, or an optionally substituted tautomer of A, T, C, G or U.
   54. The oligonucleotide of any one of the preceding Embodiments, wherein the length of the second domain is about 10-25 nucleobases.
   55. The oligonucleotide of any one of the preceding Embodiments, wherein about 10-25 nucleobases in the second domain are each independently optionally substituted A, T, C, G, or U, or an optionally substituted tautomer of A, T, C, G or U.
   56. The oligonucleotide of any one of the preceding Embodiments, wherein the length of the second domain is about 21 or more nucleobases.
   57. The oligonucleotide of any one Embodiments 1-55, wherein the length ofthe second domain is about 20 nucleobases.
   58. The oligonucleotide of any one Embodiments 1-55, wherein the length ofthe second domain is about 19 nucleobases.
   59. The oligonucleotide of any one Embodiments 1-55, wherein the length ofthe second domain is about 18 nucleobases.
   60. The oligonucleotide of any one Embodiments 1-55, wherein the length ofthe second domain is about 17 nucleobases.
   61. The oligonucleotide of any one Embodiments 1-55, wherein the length ofthe second domain is about 16 nucleobases.
   62. The oligonucleotide of any one Embodiments 1-55, wherein the length ofthe second domain is about 15 nucleobases.
   63. The oligonucleotide of any one Embodiments 1-55, wherein the length ofthe second domain is about 14 nucleobases.
   64. The oligonucleotide of any one Embodiments 1-55, wherein the length ofthe second domain is about 13 nucleobases.
   65. The oligonucleotide of any one Embodiments 1-55, wherein the length ofthe second domain is about 12 nucleobases.
   66. The oligonucleotide of any one Embodiments 1-55, wherein the length ofthe second domain is about 11 nucleobases.
   67. The oligonucleotide of any one Embodiments 1-55, wherein the length ofthe second domain is about 10 nucleobases.
   68. The oligonucleotide of any one of the preceding Embodiments, wherein about 10 nucleobases in the second domain are each independently optionally substituted A, T, C, G, or U, or an optionally substituted tautomer of A, T, C, G or U.
   69. The oligonucleotide of any one Embodiments 1-66, wherein about 11 nucleobases in the second domain are each independently optionally substituted A, T, C, G, or U, or an optionally substituted tautomer of A, T, C, G or U.
   70. The oligonucleotide of any one Embodiments 1-65, wherein about 12 nucleobases in the second domain are each independently optionally substituted A, T, C, G, or U, or an optionally substituted tautomer of A, T, C, G or U.
   71. The oligonucleotide of any one Embodiments 1-64, wherein about 13 nucleobases in the second domain are each independently optionally substituted A, T, C, G, or U, or an optionally substituted tautomer of A, T, C, G or U.
   72. The oligonucleotide of any one Embodiments 1-63, wherein about 14 nucleobases in the second domain are each independently optionally substituted A, T, C, G, or U, or an optionally substituted tautomer of A, T, C, G or U.
   73. The oligonucleotide of any one Embodiments 1-62, wherein about 15 nucleobases in the second domain are each independently optionally substituted A, T, C, G, or U, or an optionally substituted tautomer of A, T, C, G or U.
   74. The oligonucleotide of any one Embodiments 1-61, wherein about 16 nucleobases in the second domain are each independently optionally substituted A, T, C, G, or U, or an optionally substituted tautomer of A, T, C, G or U.
   75. The oligonucleotide of any one Embodiments 1-60, wherein about 17 nucleobases in the second domain are each independently optionally substituted A, T, C, G, or U, or an optionally substituted tautomer of A, T, C, G or U.
   76. The oligonucleotide of any one Embodiments 1-59, wherein about 18 nucleobases in the second domain are each independently optionally substituted A, T, C, G, or U, or an optionally substituted tautomer of A, T, C, G or U.
   77. The oligonucleotide of any one Embodiments 1-58, wherein about 19 nucleobases in the second domain are each independently optionally substituted A, T, C, G, or U, or an optionally substituted tautomer of A, T, C, G or U.
   78. The oligonucleotide of any one Embodiments 1-57, wherein about 20 nucleobases in the second domain are each independently optionally substituted A, T, C, G, or U, or an optionally substituted tautomer of A, T, C, G or U.
   79. The oligonucleotide of any one Embodiments 1-56, wherein about 21 or more nucleobases in the second domain are each independently optionally substituted A, T, C, G, or U, or an optionally substituted tautomer of A, T, C, G or U.
   80. The oligonucleotide of any one of the preceding Embodiments, wherein the second domain and the second portion have the same lengths.
   81. The oligonucleotide of any one of the preceding Embodiments, wherein the base sequence of the second domain is about 80%-100% complementary to the base sequence of the second portion.
   82. The oligonucleotide of any one of the preceding Embodiments, wherein the base sequence of the second domain comprises a mismatch with the base sequence of the second portion.
   83. The oligonucleotide of any one of the preceding Embodiments, wherein the base sequence of the second domain comprises a wobble with the base sequence of the second portion.
   84. The oligonucleotide of any one of the preceding Embodiments, wherein the base sequence of the second domain comprises a bulge with the base sequence of the second portion.
   85. The oligonucleotide of any one of the preceding Embodiments, wherein a nucleobase opposite to a target adenine is not T or U.
   86. The oligonucleotide of any one of the preceding Embodiments, wherein the base sequence of the second domain is about or at least about 85% complementary to the base sequence of the second portion.
   87. The oligonucleotide of any one of the preceding Embodiments, wherein the base sequence of the second domain is about or at least about 90% complementary to the base sequence of the second portion.
   88. The oligonucleotide of any one of the preceding Embodiments, wherein the base sequence of the second domain is about or at least about 95% complementary to the base sequence of the second portion.
   89. The oligonucleotide of any one of the preceding Embodiments, wherein the first nucleobase from the 5′-end of the second domain is complementary to a nucleobase in the second portion.
   90. The oligonucleotide of any one of the preceding Embodiments, wherein the first nucleobase from the 3′-end of the second domain is complementary to a nucleobase in the second portion.
   91. The oligonucleotide of any one of the preceding Embodiments, wherein the first nucleobase from the 5′-end of the first domain is complementary to a nucleobase in the first portion.
   92. The oligonucleotide of any one of the preceding Embodiments, wherein the first nucleobase from the 3′-end of the first domain is complementary to a nucleobase in the first portion.
   93. The oligonucleotide of any one of Embodiments 1-81, wherein the base sequence of the second domain is 100% complementary to the base sequence of the second portion.
   94. The oligonucleotide of any one of the preceding Embodiments, wherein when the oligonucleotide is administered or delivered to a system comprising a target nucleic acid or a fragment thereof that comprises the second portion, the second domain can selectively form a duplex with the second portion.
   95. The oligonucleotide of any one of the preceding Embodiments, wherein when the oligonucleotide is administered or delivered to a system expressing a target nucleic acid or a fragment thereof that comprises the second portion, the second domain can selectively form a duplex with the second portion.
   96. The oligonucleotide of any one of the preceding Embodiments, wherein the base sequence of the second portion differentiates the second portion from the rest of the target nucleic acid.
   97. The oligonucleotide of any one of the preceding Embodiments, wherein the base sequence of the second portion differentiates the second portion from the other transcripts in a system.
   98. The oligonucleotide of any one of the preceding Embodiments, wherein the base sequence of the second portion differentiates the second portion from the other RNA in a system.
   99. The oligonucleotide of any one of the preceding Embodiments, wherein the base sequence of the second portion differentiates the second portion from the other nucleic acids in a system.
   100. The oligonucleotide of any one of the preceding Embodiments, wherein the length of the gap is about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 nucleobases.
   101. The oligonucleotide of any one of the preceding Embodiments, wherein the length of the gap is about or at least about 3 nucleobases.
   102. The oligonucleotide of any one of the preceding Embodiments, wherein the length of the gap is about or at least about 4 nucleobases.
   103. The oligonucleotide of any one of the preceding Embodiments, wherein the length of the gap is about or at least about 5 nucleobases.
   104. The oligonucleotide of any one of the preceding Embodiments, wherein the length of the gap is about or at least about 6 nucleobases.
   105. The oligonucleotide of any one of the preceding Embodiments, wherein the length of the gap is about or at least about 7 nucleobases.
   106. The oligonucleotide of any one of the preceding Embodiments, wherein the length of the gap is about or at least about 8 nucleobases.
   107. The oligonucleotide of any one of the preceding Embodiments, wherein the length of the gap is about or at least about 9 nucleobases.
   108. The oligonucleotide of any one of the preceding Embodiments, wherein the length of the gap is about or at least about 10 nucleobases.
   109. The oligonucleotide of any one of the preceding Embodiments, wherein the length of the gap is about or at least about 11 nucleobases.
   110. The oligonucleotide of any one of the preceding Embodiments, wherein the length of the gap is about or at least about 12 nucleobases.
   111. The oligonucleotide of any one of the preceding Embodiments, wherein the length of the gap is about or at least about 13 nucleobases.
   112. The oligonucleotide of any one of the preceding Embodiments, wherein the length of the gap is about or at least about 14 nucleobases.
   113. The oligonucleotide of any one of the preceding Embodiments, wherein the length of the gap is about or at least about 15 nucleobases.
   114. The oligonucleotide of any one of the preceding Embodiments, wherein the length of the gap is about or at least about 16 nucleobases.
   115. The oligonucleotide of any one of the preceding Embodiments, wherein the length of the gap is about or at least about 17 nucleobases.
   116. The oligonucleotide of any one of the preceding Embodiments, wherein the length of the gap is about or at least about 18 nucleobases.
   117. The oligonucleotide of any one of the preceding Embodiments, wherein the length of the gap is about or at least about 19 nucleobases.
   118. The oligonucleotide of any one of the preceding Embodiments, wherein the length of the gap is about or at least about 20 nucleobases.
   119. The oligonucleotide of any one of the preceding Embodiments, wherein the length of the gap is about or at least about 25 nucleobases.
   120. The oligonucleotide of any one of the preceding Embodiments, wherein the length of the gap is about or at least about 30 nucleobases.
   121. The oligonucleotide of any one of the preceding Embodiments, wherein the length of the gap is about or at least about 40 nucleobases.
   122. The oligonucleotide of any one of the preceding Embodiments, wherein the length of the gap is about or at least about 50 nucleobases.
   123. The oligonucleotide of any one of the preceding Embodiments, wherein the length of the gap is about or at least about 100 nucleobases.
   124. The oligonucleotide of any one of the preceding Embodiments, wherein the length of the gap is about or at least about 200 nucleobases.
   125. The oligonucleotide of any one of the preceding Embodiments, wherein the length of the gap is about or at least about 500 nucleobases.
   126. The oligonucleotide of any one of the preceding Embodiments, wherein the length of the gap is about or at least about 1000 nucleobases.
   127. The oligonucleotide of any one of the preceding Embodiments, wherein the length of the gap is about or at least about 2000 nucleobases.
   128. The oligonucleotide of any one of the preceding Embodiments, wherein the length of the gap is about or at least about 5000 nucleobases.
   129. The oligonucleotide of any one of the preceding Embodiments, wherein the length of the gap is about or at least about 10000 nucleobases.
   130. The oligonucleotide of any one of the preceding Embodiments, wherein the length of the gap is about 2-100,000, 2-50,000, 2-10,000, 2-5,000, 3-100,000, 3-50,000, 3-10,000, 3-5,000, 4-100,000, 4-50,000, 4-10,000, 4-5,000, 5-100,000, 5-50,000, 5-10,000, 5-5,000, 10-100,000, 10-50,000, 10-10,000, 10-5,000, 20-100,000, 20-50,000, 20-10,000, 20-5,000, 50-100,000, 50-50,000, 50-10,000, 50-5,000, 100-100,000, 100-50,000, 100-10,000, or 100-5,000 nucleobases.
   131. The oligonucleotide of any one of the preceding Embodiments, wherein each nucleobase in the gap is independently optionally substituted A, T, C, G, or U, or an optionally substituted tautomer of A, T, C, G or U.
   132. The oligonucleotide of any one of the preceding Embodiments, wherein each nucleobase in the gap is independently optionally substituted A, T, C, G, or U.
   133. The oligonucleotide of any one of the preceding Embodiments, wherein each nucleobase in the gap is independently A, T, C, G, or U.
   134. The oligonucleotide of any one of the preceding Embodiments, wherein the first domain is to the 5′-side of the second domain.
   135. The oligonucleotide of any one of the preceding Embodiments, wherein the first domain is to the 3′-side of the second domain.
   136. The oligonucleotide of any one of the preceding Embodiments, wherein the first domain is connected to the second domain through a linker.
   137. The oligonucleotide of any one of the preceding Embodiments, wherein the linker is or comprises a nucleoside.
   138. The oligonucleotide of any one of the preceding Embodiments, wherein the linker is or comprises an oligonucleotide (“linker oligonucleotide”).
   139. The oligonucleotide of any one of the preceding Embodiments, wherein the length of the linker oligonucleotide is about or at least about 2-100, 2-90, 2-80, 2-70, 2-60, 2-50, 2-40, 2-30, 2-20, 2-10, 2, 3, 4, 5, 6, 7, 8, 9, 10, 11, 12, 13, 14, 15, 16, 17, 18, 19, or 20 nucleobases.
   140. The oligonucleotide of any one of the preceding Embodiments, wherein the complementarity of the linker oligonucleotide to the gap in the target nucleic acid is no more than about 90%, 85%, 80%, 75%, 70%, 65%, 60%, 50%, 40%, 35%, 30%, 25%, 20%, 10%, or 5%.
   141. The oligonucleotide of any one of the preceding Embodiments, wherein the complementarity of the linker oligonucleotide to the gap in the target nucleic acid is no more than about 85%.
   142. The oligonucleotide of any one of the preceding Embodiments, wherein the complementarity of the linker oligonucleotide to the gap in the target nucleic acid is no more than about 80%.
   143. The oligonucleotide of any one of the preceding Embodiments, wherein the complementarity of the linker oligonucleotide to the gap in the target nucleic acid is no more than about 75%.
   144. The oligonucleotide of any one of the preceding Embodiments, wherein the complementarity of the linker oligonucleotide to the gap in the target nucleic acid is no more than about 70%.
   145. The oligonucleotide of any one of the preceding Embodiments, wherein the complementarity of the linker oligonucleotide to the gap in the target nucleic acid is no more than about 65%.
   146. The oligonucleotide of any one of the preceding Embodiments, wherein the complementarity of the linker oligonucleotide to the gap in the target nucleic acid is no more than about 60%.
   147. The oligonucleotide of any one of the preceding Embodiments, wherein the complementarity of the linker oligonucleotide to the gap in the target nucleic acid is no more than about 55%.
   148. The oligonucleotide of any one of the preceding Embodiments, wherein the complementarity of the linker oligonucleotide to the gap in the target nucleic acid is no more than about 50%.
   149. The oligonucleotide of any one of the preceding Embodiments, wherein the complementarity of the linker oligonucleotide to the gap in the target nucleic acid is no more than about 40%,
   150. The oligonucleotide of any one of the preceding Embodiments, wherein the complementarity of the linker oligonucleotide to the gap in the target nucleic acid is no more than about 35%.
   151. The oligonucleotide of any one of the preceding Embodiments, wherein the complementarity of the linker oligonucleotide to the gap in the target nucleic acid is no more than about 30%,
   152. The oligonucleotide of any one of the preceding Embodiments, wherein the complementarity of the linker oligonucleotide to the gap in the target nucleic acid is no more than about 25%.
   153. The oligonucleotide of any one of the preceding Embodiments, wherein the complementarity of the linker oligonucleotide to the gap in the target nucleic acid is no more than about 20%,
   154. The oligonucleotide of any one of the preceding Embodiments, wherein the complementarity of the linker oligonucleotide to the gap in the target nucleic acid is no more than about 10%,
   155. The oligonucleotide of any one of the preceding Embodiments, wherein the complementarity of the linker oligonucleotide to the gap in the target nucleic acid is no more than about 5%.
   156. The oligonucleotide of any one of the preceding Embodiments, wherein the linker comprises polyvinylether, polyethylene, polypropylene, polyethylene glycol (PEG), polypropylene glycol (PEG), polyvinyl alcohol (PVA), polyglycolide (PGA), polylactide (PLA), polycaprolactone (PCL), or copolymers thereof.
   157. The oligonucleotide of any one of the preceding Embodiments, wherein the linker is, comprises or consists of one or more units each independently selected from

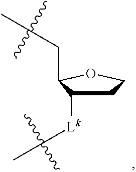

—(CH₂)ₙ-L^{k}-, and —(CH₂CH₂O),CH₂CH₂-L^{k}-. wherein each n is independently about 1-100, 1-50, 1-40, 1-30, 1-20, 1-10, 1-5, 2-100, 2-50, 2-40, 2-30, 2-20, 2-10, 2-5, 3-100, 3-50, 3-40, 3-30, 3-20, 3-10, 3-5, 1, 2, 3, 4, 5, 6, 7, 8, 9, 10, 11, 12, 13. 14, 15, 16, 17, 18, 19, or 20, and each L^{k} independently has the structure of internucleotidic linkage.
158. The oligonucleotide of any one of the preceding Embodiments, wherein the linker comprises one or more units each independently selected from

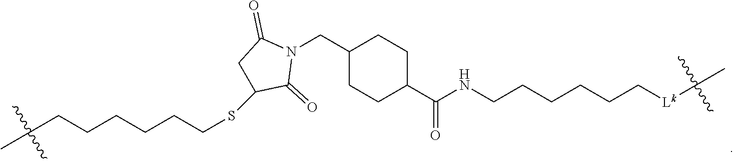

159. The oligonucleotide of any one of Embodiments 157-158, wherein each L^{k} independently has the structure of a PO, PS or PN linkage.
160. The oligonucleotide of any one of Embodiments 157-158, wherein each L^{k} independently has the structure of a PO, PS or phosphoryl guanidine linkage.
161. The oligonucleotide of any one of Embodiments 157-160, wherein at least one L^{k} is has the structure of a natural phosphate linkage.
162. The oligonucleotide of any one of Embodiments 157-161, wherein at least one L^{k} has the structure of phosphorothioate internucleotidic linkage.
163. The oligonucleotide of any one of Embodiments 157-162, wherein at least one L^{k} has the structure of a PN linkage.
164. The oligonucleotide of any one of Embodiments 157-162, wherein at least one L^{k} has the structure of a phosphoryl guanidine linkage.
165. The oligonucleotide of any one of Embodiments 157-162, wherein at least one L^{k} has the structure of n001.
166. The oligonucleotide of any one of Embodiments 157-165, wherein each L^{k} independently has the structure of a natural phosphate linkage, a phosphorothioate internucleotidic linkage or a phosphoryl guanidine internucleotidic linkage.
167. The oligonucleotide of any one of Embodiments 157-166, wherein each L^{k} independently has the structure of a natural phosphate linkage, a phosphorothioate internucleotidic linkage or n001.
168. The oligonucleotide of any one of the preceding Embodiments, wherein the linker is, comprises or consists of one or more units each independently selected from

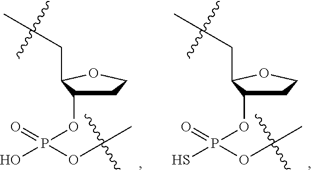

—(CH₂)ₙOP(O)(OH)O—, —(CH₂)˜OP(O)(SH)O—, —(CH₂CH₂O)ₙP(O)(OH)O—, and -(CH₂CH₂O)ₙP(O)(SH)O—, wherein each n is independently about 1-100, 1-50, 1-40, 1-30, 1-20, 1-10, 1-5, 2-100, 2-50, 2-40, 2-30, 2-20, 2-10, 2-5, 3-100, 3-50, 3-40, 3-30, 3-20, 3-10, 3-5, 1, 2, 3, 4, 5, 6, 7, 8, 9, 10, 11, 12, 13, 14, 15, 16, 17, 18, 19, or 20.
169. The oligonucleotide of any one of Embodiments 157-168, wherein n is about 1-20.
170. The oligonucleotide of any one of Embodiments 157-168, wherein n is about 3-10.
171. The oligonucleotide of any one of the preceding Embodiments, wherein the linker comprises one or more units each independently selected from

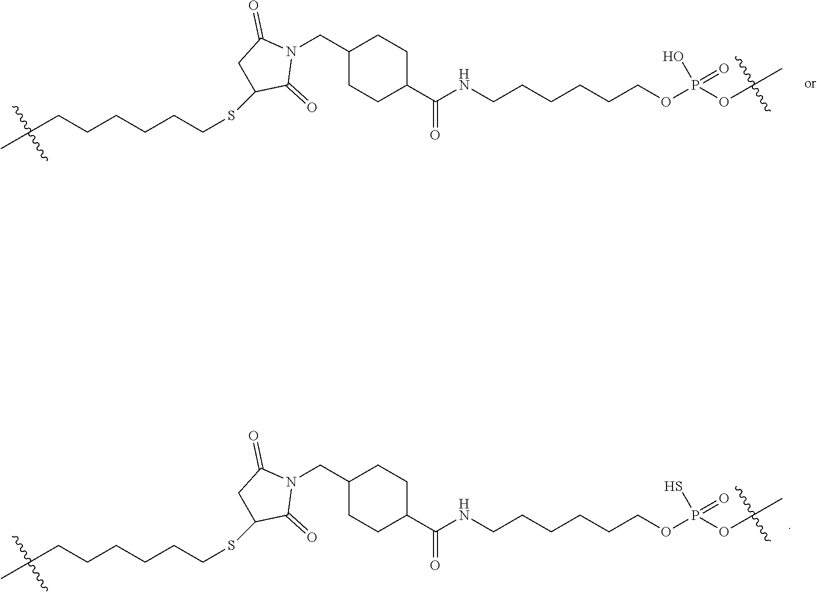 [SMILES: *CCCCCCSC1CC(=O)N(CC2CCC(C(=O)NCCCCCCOP(=O)(O)O*)CC2)C1=O ; *CCCCCCSC1CC(=O)N(CC2CCC(C(=O)NCCCCCCOP(=O)(S)O*)CC2)C1=O]

172. The oligonucleotide of any one of Embodiments 157-171, wherein for one unit, L^{k}, or the phosphate or phosphorothioate moiety is bonded to 5′-position of a 5′-end nucleoside of the first domain.
173. The oligonucleotide of any one of Embodiments 157-171, wherein for one unit, L^{k}, or the phosphate or phosphorothioate moiety is bonded to 3′-position of a 3′-end nucleoside of the first domain.
174. The oligonucleotide of any one of Embodiments 157-171, wherein for one unit, L^{k}, or the phosphate or phosphorothioate moiety is bonded to 5′-position of a 5′-end nucleoside of the second domain.
175. The oligonucleotide of any one of Embodiments 157-171, wherein for one unit, L^{k}, or the phosphate or phosphorothioate moiety is bonded to 3′-position of a 3′-end nucleoside of the second domain.
176. The oligonucleotide of any one of Embodiments 157-175, wherein for one unit, the —CH₂- is bonded to 5′-position of a 5′-end nucleoside of the first domain through L^{k}.
177. The oligonucleotide of any one of Embodiments 157-175, wherein for one unit, the —CH₂- is bonded to 3′-position of a 3′-end nucleoside of the first domain through L^{k}.
178. The oligonucleotide of any one of Embodiments 157-175, wherein for one unit, the —CH₂- is bonded to 5′-position of a 5′-end nucleoside of the second domain through L^{k}.
179. The oligonucleotide of any one of Embodiments 157-175, wherein for one unit, the —CH₂- is bonded to 3′-position of a 3′-end nucleoside of the second domain through L^{k}.
180. The oligonucleotide of any one of Embodiments 176-179, wherein L^{k} has the structure of a PO linkage.
181. The oligonucleotide of any one of Embodiments 176-179, wherein L^{k} has the structure of a natural phosphate linkage.
182. The oligonucleotide of any one of Embodiments 176-179, wherein L^{k} has the structure of a PS linkage.
183. The oligonucleotide of any one of Embodiments 176-179, wherein L^{k} has the structure of a phosphorothioate internucleotidic linkage.
184. The oligonucleotide of any one of Embodiments 176-179, wherein L^{k} has the structure of a PN linkage.
185. The oligonucleotide of any one of Embodiments 176-179, wherein L^{k} has the structure of a phosphoryl guanidine linkage.
186. The oligonucleotide of any one of Embodiments 176-179, wherein L^{k} has the structure of n001.
187. The oligonucleotide of any one of Embodiments 157-186, wherein the linker comprises an optionally substituted 1H-1,2,3-triazole ring.
188. The oligonucleotide of any one of Embodiments 157-186, wherein the linker comprises an optionally substituted 1H-1,2,3-triazole ring formed from an azide and an alkyne.
189. The oligonucleotide of any one of Embodiments 157-188, wherein one or more units are independently a salt form.
190. The oligonucleotide of any one of Embodiments 1-135, wherein the first domain is directly connected to the second domain.
191. The oligonucleotide of any one of the preceding Embodiments, wherein the first portion is to the 5′-side of the second portion.
192. The oligonucleotide of any one of the preceding Embodiments, wherein the first portion is to the 3′-side of the second portion.
193. The oligonucleotide of any one of the preceding Embodiments, wherein the target nucleic acid is RNA.
194. The oligonucleotide of any one of the preceding Embodiments, wherein the target nucleic acid is a transcript.
195. The oligonucleotide of any one of the preceding Embodiments, wherein the target nucleic acid is mRNA.
196. The oligonucleotide of any one of the preceding Embodiments, wherein the target nucleic acid comprises a target adenosine which is a G to A mutation.
197. The oligonucleotide of any one of the preceding Embodiments, wherein a system is in vitro.
198. The oligonucleotide of any one of the preceding Embodiments, wherein a system is ex vivo.
199. The oligonucleotide of any one of the preceding Embodiments, wherein a system is or comprises a cell.
200. The oligonucleotide of any one of the preceding Embodiments, wherein a system is or comprises a tissue.
201. The oligonucleotide of any one of the preceding Embodiments, wherein a system is or comprises an organ.
202. The oligonucleotide of any one of the preceding Embodiments, wherein a system is or comprises an organism.
203. The oligonucleotide of any one of the preceding Embodiments, wherein a system is a subject.
204. The oligonucleotide of any one of the preceding Embodiments, comprising a modified nucleobase, nucleoside, sugar or internucleotidic linkage as described in the present disclosure.
205. The oligonucleotide of any one of the preceding Embodiments, wherein about or at least about 60%, 65%, 70%, 75%, 80%, 85%, 90%, or 95% of all sugars are 2′-F modified sugars.
206. The oligonucleotide of any one of the preceding Embodiments, comprising one or more modified sugars and/or one or more modified internucleotidic linkages.
207. The oligonucleotide of any one of Embodiments 1-206, wherein when the oligonucleotide is contacted with a target nucleic acid comprising a target adenosine in a system, a target adenosine in the target nucleic acid is modified.
208. The oligonucleotide of any one of Embodiments 1-206, wherein when the oligonucleotide is contacted with a target nucleic acid comprising a target adenosine in a system, level of the target nucleic acid is reduced compared to absence of the product or presence of a reference oligonucleotide.
209. The oligonucleotide of any one of Embodiments 1-206, wherein when the oligonucleotide is contacted with a target nucleic acid comprising a target adenosine in a system, splicing of the target nucleic acid or a product thereof is altered compared to absence of the oligonucleotide or presence of a reference oligonucleotide.
210. The oligonucleotide of any one of Embodiments 1-206, wherein when the oligonucleotide is contacted with a target nucleic acid comprising a target adenosine in a system, level of a product of the target nucleic acid is altered compared to absence of the product or presence of a reference oligonucleotide.
211. The oligonucleotide of any one of Embodiments 208-210, wherein the target nucleic acid is modified.
212. The oligonucleotide of any one of Embodiments 207-211, wherein level of a product is increased, wherein the product is or is encoded by a nucleic acid which is otherwise identical to the target nucleic acid but the target adenosine is modified.
213. The oligonucleotide of any one of Embodiments 207-211, wherein level of a product is increased. wherein the product is or is encoded by a nucleic acid which is otherwise identical to the target nucleic acid but the target adenosine is replaced with inosine.
214. The oligonucleotide of any one of Embodiments 207-211, wherein level of a product is increased, wherein the product is or is encoded by a nucleic acid which is otherwise identical to the target nucleic acid but the adenine of the target adenosine is replaced with guanine.
215. The oligonucleotide of any one of Embodiments 212-214, wherein the product is a protein.
216. The oligonucleotide of any one of the preceding Embodiments, wherein the target adenosine is a mutation from guanine.
217. The oligonucleotide of any one of the preceding Embodiments, wherein the target adenosine is more associated with a condition, disorder or disease than a guanine at the same position.
218. The oligonucleotide of any one of the preceding Embodiments, wherein the oligonucleotide is capable of forming a double-stranded complex with the target nucleic acid.
219. The oligonucleotide of Embodiment 207-218, wherein a target nucleic acid or a portion thereof is or comprises RNA.
220. The oligonucleotide of any one of Embodiments 207-219, wherein the target adenosine is of an RNA.
221. The oligonucleotide of any one of Embodiments 207-220, wherein the target adenosine is modified, and the modification is or comprises deamination of the target adenosine.
222. The oligonucleotide of any one of Embodiments 207-221, wherein the target adenosine is modified and the modification is or comprises conversion of the target adenosine to an inosine.
223. The oligonucleotide of any one of Embodiments 207-222, wherein the modification is promoted by an ADAR protein.
224. The oligonucleotide of any one of Embodiments 207-223, wherein the system is an in vitro or ex vivo system comprising an ADAR protein.
225. The oligonucleotide of any one of Embodiments 207-223, wherein the system is or comprises a cell that comprises or expresses an ADAR protein.
226. The oligonucleotide of any one of Embodiments 207-223, wherein the system is a subject comprising a cell that comprises or expresses an ADAR protein.
227. The oligonucleotide of any one of Embodiments 223-226, wherein the ADAR protein is ADAR1.
228. The oligonucleotide of any one of Embodiments 223-226, wherein the ADAR protein is ADAR2.
229. The oligonucleotide of any one of the preceding Embodiments, wherein the oligonucleotide has a length of about 10-200 (e.g., about 10-20, 10-30, 10-40, 10-50, 10-60, 10-70, 10-80, 10-90, 10-100, 10-120, 10-150, 20-30, 20-40, 20-50, 20-60, 20-70, 20-80, 20-90, 20-100, 20-120, 20-150, 20-200, 25-30, 25-40, 25-50, 25-60, 25-70, 25-80, 25-90, 25-100, 25-120, 25-150, 25-200, 30-40, 30-50, 30-60, 30-70, 30-80, 30-90, 30-100, 30-120, 30-150, 30-200, 10, 20, 25, 26, 27, 28, 29, 30, 31, 32, 33, 34, 35, 36, 37, 38, 39, 40, 45, 50, 60, etc.) nucleobases.
230. The oligonucleotide of any one of the preceding Embodiments, wherein the oligonucleotide has a length of about 20-50 nucleobases.
231. The oligonucleotide of any one of the preceding Embodiments, wherein the oligonucleotide has a length of about 30-40 nucleobases.
232. The oligonucleotide of any one of the preceding Embodiments, wherein the oligonucleotide has a length of about 26-35 nucleobases.
233. The oligonucleotide of any one of the preceding Embodiments, wherein the oligonucleotide has a length of about 26 nucleobases.
234. The oligonucleotide of any one of the preceding Embodiments, wherein the oligonucleotide has a length of about 27 nucleobases.
235. The oligonucleotide of any one of the preceding Embodiments, wherein the oligonucleotide has a length of about 28 nucleobases.
236. The oligonucleotide of any one of the preceding Embodiments, wherein the oligonucleotide has a length of about 29 nucleobases.
237. The oligonucleotide of any one of the preceding Embodiments, wherein the oligonucleotide has a length of about 30 nucleobases.
238. The oligonucleotide of any one of the preceding Embodiments, wherein the oligonucleotide has a length of about 31 nucleobases.
239. The oligonucleotide of any one of the preceding Embodiments, wherein the oligonucleotide has a length of about 32 nucleobases.
240. The oligonucleotide of any one of the preceding Embodiments, wherein the oligonucleotide has a length of about 33 nucleobases.
241. The oligonucleotide of any one of the preceding Embodiments, wherein the oligonucleotide has a length of about 34 nucleobases.
242. The oligonucleotide of any one of the preceding Embodiments, wherein the oligonucleotide has a length of about 35 nucleobases.
243. The oligonucleotide of any one of the preceding Embodiments, wherein the oligonucleotide consists of a first domain and a second domain.
244. The oligonucleotide of any one of the preceding Embodiments, wherein the first domain has a length of about 2-50 (e.g., about 5, 6, 7, 8, 9, or 10-about 10, 11, 12, 13, 14, 15, 16, 17, 18, 19, 20, 21, 22. 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
245. The oligonucleotide of any one of the preceding Embodiments, wherein the first domain has a length of about 10-25 nucleobases.
246. The oligonucleotide of any one of the preceding Embodiments, wherein the first domain has a length of about 15 nucleobases.
247. The oligonucleotide of any one of the preceding Embodiments, wherein the first domain comprises one or more (e.g., 1-10, 1, 2, 3, 4, 5, 6, 7, 8, 9, or 10, etc.) mismatches when the oligonucleotide is aligned with a target nucleic acid for complementarity.
248. The oligonucleotide of any one of the preceding Embodiments, wherein the first domain comprises two or more mismatches when the oligonucleotide is aligned with a target nucleic acid for complementarity.
249. The oligonucleotide of any one of Embodiments 1-244, wherein the first domain comprises one and no more than one mismatch when the oligonucleotide is aligned with a target nucleic acid for complementarity.
250. The oligonucleotide of any one of Embodiments 1-244, wherein the first domain comprises two and no more than two mismatches when the oligonucleotide is aligned with a target nucleic acid for complementarity.
251. The oligonucleotide of any one of the preceding Embodiments, wherein the first domain comprises one or more (e.g., 1-10, 1, 2, 3, 4, 5, 6, 7, 8, 9, or 10, etc.) bulges when the oligonucleotide is aligned with a target nucleic acid for complementarity.
252. The oligonucleotide of Embodiment 251, wherein each bulge independently comprises one or more base pairs that are not Watson-Crick or wobble pairs.
253. The oligonucleotide of any one of the preceding Embodiments, wherein the first domain comprises one or more (e.g., 1-10, 1, 2, 3, 4, 5, 6, 7, 8, 9, or 10, etc.) wobble pairs when the oligonucleotide is aligned with a target nucleic acid for complementarity.
254. The oligonucleotide of any one of the preceding Embodiments, wherein the first domain comprises two or more wobble pairs when the oligonucleotide is aligned with a target nucleic acid for complementarity.
255. The oligonucleotide of any one of the preceding Embodiments, wherein the first domain comprises two and no more than two wobble pairs when the oligonucleotide is aligned with a target nucleic acid for complementarity.
256. The oligonucleotide of any one of the preceding Embodiments, wherein the first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) sugars with 2′-F modification.
257. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the first domain independently comprise a 2-F modification.
258. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first domain independently comprise a 2′-F modification.
259. The oligonucleotide of any one of the preceding Embodiments, wherein about 30%-70% (e.g., about 30%-60%, 30%-50%, or about 30%, 40%, 50%, 60% or 70%) of sugars in the first domain independently comprise a 2′-F modification.
260. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OMe.
261. The oligonucleotide of any one of the preceding Embodiments, wherein about 30%-70% (e.g., about 30%-60%, 30%-50%, or about 30%, 40%, 50%, 60% or 70%) of sugars in the first domain comprises 2′-OMe.
262. The oligonucleotide of any one of the preceding Embodiments, wherein no more than about 50% of sugars in the first domain comprises 2′-OMe.
263. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OR, wherein R is optionally substituted C₁₋₆ aliphatic.
264. The oligonucleotide of any one of the preceding Embodiments, wherein about 30%-70% (e.g., about 30%-60%, 30%-50%, or about 30%, 40%, 50%, 60% or 70%) of sugars in the first domain comprises 2′-OR, wherein R is optionally substituted C₁₋₆ aliphatic.
265. The oligonucleotide of any one of the preceding Embodiments, wherein no more than about 50% of sugars in the first domain comprises 2′-OR, wherein R is optionally substituted C₁₋₆ aliphatic.
266. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OR.
267. The oligonucleotide of any one of the preceding Embodiments, wherein about 30%-70% (e.g., about 30%-60%, 30%-50%, or about 30%, 40%, 50%, 60% or 70%) of sugars in the first domain comprises 2′-OR, wherein R is not —H.
268. The oligonucleotide of any one of the preceding Embodiments, wherein no more than about 50% of sugars in the first domain comprises 2′-OR.
269. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
270. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-MOE modification.
271. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
272. The oligonucleotide of any one of the preceding Embodiments, wherein the first about 1-5, e.g., 1, 2, 3, 4, or 5 sugars from the 5′-end of a first domain is independently a 2′-OR modified sugar, wherein R is independently optionally substituted C₁₋₆ aliphatic.
273. The oligonucleotide of any one of the preceding Embodiments, wherein the first about 1-5, e.g., 1, 2, 3, 4, or 5 sugars from the 5′-end of a first domain is independently a 2′-MOE modified sugar.
274. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
275. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
276. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
277. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
278. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
279. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
280. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
281. The oligonucleotide of any one of Embodiments 1-268, wherein no sugar in the first domain comprises 2′-OR.
282. The oligonucleotide of any one of Embodiments 1-268, wherein no sugar in the first domain comprises 2′-OMe.
283. The oligonucleotide of any one of Embodiments 1-268, wherein no sugar in the first domain comprises 2′-OR, wherein R is optionally substituted C₁₋₆ aliphatic.
284. The oligonucleotide of any one of Embodiments 1-268, wherein each sugar in the first domain comprises 2′-F.
285. The oligonucleotide of any one of the preceding Embodiments, wherein the first domain comprise about 1-50 (e.g., about 5, 6, 7, 8, 9, or 10-about 10, 11, 12. 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
286. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the first domain are modified internucleotidic linkages.
287. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the first domain are modified internucleotidic linkages.
288. The oligonucleotide of any one of the preceding Embodiments, wherein each modified internucleotidic linkages is independently a chiral internucleotidic linkage.
289. The oligonucleotide of any one of the preceding Embodiments, wherein each modified internucleotidic linkages is independently a phosphorothioate internucleotidic linkage or a non-negatively charged internucleotidic linkage.
290. The oligonucleotide of any one of the preceding Embodiments, wherein each modified internucleotidic linkages is independently a phosphorothioate internucleotidic linkage or a neutral internucleotidic linkage.
291. The oligonucleotide of any one of the preceding Embodiments, wherein the first domain comprises one or more phosphorothioate internucleotidic linkages.
292. The oligonucleotide of any one of the preceding Embodiments, wherein the first domain comprises 1, 2, 3, 4, or 5 non-negatively charged internucleotidic linkages.
293. The oligonucleotide of any one of the preceding Embodiments, wherein the internucleotidic linkage between the first and the second nucleosides of the first domain is a non-negatively charged internucleotidic linkage.
294. The oligonucleotide of any one of the preceding Embodiments, wherein the internucleotidic linkage between the last and the second last nucleosides of the first domain is a non-negatively charged internucleotidic linkage.
295. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first domain is chirally controlled.
296. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first domain is chirally controlled.
297. The oligonucleotide of any one of the preceding Embodiments, wherein the internucleotidic linkage between the first and the second nucleosides of the first domain is chirally controlled.
298. The oligonucleotide of any one of the preceding Embodiments, wherein the internucleotidic linkage between the last and the second last nucleosides of the first domain is chirally controlled.
299. The oligonucleotide of any one of the preceding Embodiments, wherein each chiral internucleotidic linkage is independently a chirally controlled internucleotidic linkage.
300. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first domain is Sp.
301. The oligonucleotide of any one of the preceding Embodiments, wherein at least 5%-100% (e.g.. about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first domain is Sp.
302. The oligonucleotide of any one of the preceding Embodiments, wherein each chiral internucleotidic linkages in the first domain is Sp.
303. The oligonucleotide of any one of Embodiments 1-301, wherein the internucleotidic linkage between the first and the second nucleosides of the first domain is Rp.
304. The oligonucleotide of any one of Embodiments 1-301 and 303, wherein the internucleotidic linkage between the last and the second last nucleosides of the first domain is Rp.
305. The oligonucleotide of any one of the preceding Embodiments, wherein each internucleotidic linkage in the first domain is independently a modified internucleotidic linkage.
306. The oligonucleotide of any one of Embodiments 1-304, wherein the first domain comprises one or more natural phosphate linkages.
307. The oligonucleotide of any one of the preceding Embodiments, wherein the first domain can recruit, or promotes or contributes to recruitment of, an ADAR protein to a target nucleic acid.
308. The oligonucleotide of any one of the preceding Embodiments, wherein the first domain can interact, or promotes or contributes to interaction of, an ADAR protein with a target nucleic acid.
309. The oligonucleotide of any one of the preceding Embodiments, wherein the first domain contacts with a RNA binding domain (RBD) of ADAR.
310. The oligonucleotide of any one of the preceding Embodiments, wherein the first domain does not substantially contact with a second RBD domain of ADAR.
311. The oligonucleotide of any one of the preceding Embodiments, wherein the first domain does not substantially contact with a catalytic domain which has a deaminase activity, of ADAR.
312. The oligonucleotide of any one of the preceding Embodiments, wherein the second domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
313. The oligonucleotide of any one of the preceding Embodiments, wherein the second domain has a length of about 1-20 nucleobases.
314. The oligonucleotide of any one of the preceding Embodiments, wherein the second domain has a length of about 5-15 nucleobases.
315. The oligonucleotide of any one of the preceding Embodiments, wherein the second domain has a length of about 10-25 nucleobases.
316. The oligonucleotide of any one of the preceding Embodiments, wherein the second domain has a length of about 15 nucleobases.
317. The oligonucleotide of any one of the preceding Embodiments, wherein the second domain comprises one or more (e.g., 1-10, 1, 2, 3, 4, 5, 6, 7, 8, 9, or 10, etc.) mismatches when the oligonucleotide is aligned with a target nucleic acid for complementarity.
318. The oligonucleotide of any one of the preceding Embodiments, wherein the second domain comprises two or more mismatches when the oligonucleotide is aligned with a target nucleic acid for complementarity.
319. The oligonucleotide of any one of Embodiments 1-312, wherein the second domain comprises one and no more than one mismatch when the oligonucleotide is aligned with a target nucleic acid for complementarity.
320. The oligonucleotide of any one of Embodiments 1-312, wherein the second domain comprises two and no more than two mismatches when the oligonucleotide is aligned with a target nucleic acid for complementarity.
321. The oligonucleotide of any one of the preceding Embodiments, wherein the second domain comprises one or more (e.g., 1-10, 1, 2, 3, 4, 5, 6, 7, 8, 9, or 10, etc.) bulges when the oligonucleotide is aligned with a target nucleic acid for complementarity.
322. The oligonucleotide of Embodiment 321, wherein each bulge independently comprises one or more base pairs that are not Watson-Crick or wobble pairs.
323. The oligonucleotide of any one of the preceding Embodiments, wherein the second domain comprises one or more (e.g., 1-10, 1, 2, 3, 4, 5, 6, 7, 8, 9, or 10, etc.) wobble pairs when the oligonucleotide is aligned with a target nucleic acid for complementarity.
324. The oligonucleotide of any one of the preceding Embodiments, wherein the second domain comprises two or more wobble pairs when the oligonucleotide is aligned with a target nucleic acid for complementarity.
325. The oligonucleotide of any one of the preceding Embodiments, wherein the second domain comprises two and no more than two wobble pairs when the oligonucleotide is aligned with a target nucleic acid for complementarity.
326. The oligonucleotide of any one of the preceding Embodiments, wherein the second domain comprise a nucleoside opposite to a target adenosine when the oligonucleotide is aligned with a target nucleic acid for complementarity.
327. The oligonucleotide of Embodiment 326, wherein the opposite nucleobase is optionally substituted or protected U, or is an optionally substituted or protected tautomer of U.
328. The oligonucleotide of Embodiment 326, wherein the opposite nucleobase is U.
329. The oligonucleotide of Embodiment 326, wherein the opposite nucleobase is optionally substituted or protected C, or is an optionally substituted or protected tautomer of C.
330. The oligonucleotide of Embodiment 326, wherein the opposite nucleobase is C.
331. The oligonucleotide of Embodiment 326, wherein the opposite nucleobase is optionally substituted or protected A, or is an optionally substituted or protected tautomer of A.
332. The oligonucleotide of Embodiment 326, wherein the opposite nucleobase is A.
333. The oligonucleotide of Embodiment 326, wherein the opposite nucleobase is optionally substituted or protected nucleobase of pseudoisocytosine, or is an optionally substituted or protected tautomer of the nucleobase of pseudoisocytosine.
334. The oligonucleotide of Embodiment 326, wherein the opposite nucleobase is the nucleobase of pseudoisocytosine.
335. The oligonucleotide of any one of the preceding Embodiments, wherein the oligonucleotide comprises a nucleobase BA, wherein BA is or comprises Ring BA or a tautomer thereof, wherein Ring BA is an optionally substituted, 5-20 membered, monocyclic, bicyclic or polycyclic ring having 0-10 hetereoatoms.
336. The oligonucleotide of Embodiment 326, wherein the nucleobase is BA, wherein BA is or comprises Ring BA or a tautomer thereof, wherein Ring BA is an optionally substituted, 5-20 membered, monocyclic, bicyclic or polycyclic ring having 0-10 hetereoatoms.
337. The oligonucleotide of any one of Embodiments 335-336, wherein BA has weaker hydrogen bonding with the target adenine of the adenosine compared to U.
338. The oligonucleotide of any one of Embodiments 335-337, wherein BA forms fewer hydrogen bonds with the target adenine of the adenosine compared to U.
339. The oligonucleotide of any one of Embodiments 335-338, wherein BA forms one or more hydrogen bonds with one or more amino acid residues of ADAR which residues form one or more hydrogen bonds with U opposite to a target adenosine.
340. The oligonucleotide of any one of Embodiments 335-339, wherein BA forms one or more hydrogen bonds with each amino acid residue of ADAR that forms one or more hydrogen bonds with U opposite to a target adenosine.
341. The oligonucleotide of any one of Embodiments 335-340, wherein Ring BA comprises [symbol] X² [symbol] X³
342. The oligonucleotide of any one of Embodiments 335-340, wherein Ring BA comprises [symbol] X² [symbol] X³ [symbol] X⁴ [symbol] .
343. The oligonucleotide of any one of Embodiments 335-340, wherein Ring BA comprises —X¹( [symbol] ) [symbol] X² [symbol] X³ [symbol] .
344. The oligonucleotide of any one of Embodiments 335-340, wherein Ring BA comprises -X¹( [symbol] ) [symbol] X² [symbol] X³ [symbol] X⁴ [symbol] .
345. The oligonucleotide of any one of Embodiments 335-344, wherein Ring BA has the structure of formula BA-I.
346. The oligonucleotide of any one of Embodiments 335-344, wherein Ring BA has the structure of formula BA-I-a.
347. The oligonucleotide of any one of Embodiments 335-344, wherein Ring BA has the structure of formula BA-I-b.
348. The oligonucleotide of any one of Embodiments 335-344, wherein Ring BA has the structure of formula BA-I-c.
349. The oligonucleotide of any one of Embodiments 335-344, wherein Ring BA has the structure of formula BA-I-d.
350. The oligonucleotide of any one of Embodiments 335-344, wherein Ring BA has the structure of formula BA-II.
351. The oligonucleotide of any one of Embodiments 335-344, wherein Ring BA has the structure of formula BA-II-a.
352. The oligonucleotide of any one of Embodiments 335-344, wherein Ring BA has the structure of formula BA-II-b.
353. The oligonucleotide of any one of Embodiments 335-344, wherein Ring BA has the structure of formula BA-II-c.
354. The oligonucleotide of any one of Embodiments 335-344, wherein Ring BA has the structure of formula BA-II-d.
355. The oligonucleotide of any one of Embodiments 335-344, wherein Ring BA has the structure of formula BA-III.
356. The oligonucleotide of any one of Embodiments 335-344, wherein Ring BA has the structure of formula BA-III-a.
357. The oligonucleotide of any one of Embodiments 335-344, wherein Ring BA has the structure of formula BA-III-b.
358. The oligonucleotide of any one of Embodiments 335-344, wherein Ring BA has the structure of formula BA-III-c.
359. The oligonucleotide of any one of Embodiments 335-344, wherein Ring BA has the structure of formula BA-III-d.
360. The oligonucleotide of any one of Embodiments 335-344, wherein Ring BA has the structure of formula BA-III-e.
361. The oligonucleotide of any one of Embodiments 335-360, wherein each of X¹, X², X³, X⁴, X⁵, X⁶, X¹′, X²′, X³′, X⁴′, X⁵′, X⁶′, and X⁷′ is independently and optionally substituted when it is —CH═, —C(OH)═, —C(—NH₂)═, —CH₂—, —C(═NH)—, or —NH—.
362. The oligonucleotide of any one of Embodiments 341-361, wherein X¹ is —N(−)-.
363. The oligonucleotide of any one of Embodiments 341-361, wherein X¹ is —C(−)═.
364. The oligonucleotide of any one of Embodiments 341-363, wherein X² is —C(O)—.
365. The oligonucleotide of any one of Embodiments 341-363, wherein X² is —C(S)—.
366. The oligonucleotide of any one of Embodiments 341-363, wherein X² is —C(Se)-.
367. The oligonucleotide of any one of Embodiments 341-363, wherein X² is —C(R^{B2}).
368. The oligonucleotide of any one of Embodiments 341-363, wherein X² is optionally substituted -CH═.
369. The oligonucleotide of any one of Embodiments 341-363, wherein X² is —C(OR^{B2}).
370. The oligonucleotide of any one of Embodiments 341-363, wherein X² is —C(OR^{B2})=, wherein R^{B2} is -L^{B2}-R′.
371. The oligonucleotide of any one of Embodiments 341-363, wherein X² is —N═.
372. The oligonucleotide of any one of Embodiments 341-371, wherein X³ is —N(R^{B3})_, wherein R^{B3} is -L^{B3}-R′.
373. The oligonucleotide of any one of Embodiments 341-371, wherein X³ is —NR′-.
374. The oligonucleotide of any one of Embodiments 341-371, wherein X³ is optionally substituted -NH—.
375. The oligonucleotide of any one of Embodiments 341-371, wherein X³ is —NH—.
376. The oligonucleotide of any one of Embodiments 341-371, wherein X³ is —C(R^{B2}).
377. The oligonucleotide of any one of Embodiments 341-371, wherein X³ is optionally substituted -CH═.
378. The oligonucleotide of any one of Embodiments 341-371, wherein X³ is —CH═.
379. The oligonucleotide of any one of Embodiments 341-371, wherein X³ is —N═.
380. The oligonucleotide of any one of Embodiments 341-379, wherein X⁴ is —C(R^{B4})═, —C(—N(R^{B4})₂)=, -C(R^{B4})₂, or —C(═NR^{B4})-.
381. The oligonucleotide of any one of Embodiments 341-379, wherein X⁴ is —C(R^{B4})═.
382. The oligonucleotide of any one of Embodiments 341-379, wherein X⁴ is —C(R^{B4})=, wherein R^{B4} is optionally substituted C₁₋₆ aliphatic.
383. The oligonucleotide of any one of Embodiments 341-379, wherein X⁴ is —C(R^{B4})=, wherein R^{B4} is optionally substituted C₁₋₆ alkyl.
384. The oligonucleotide of any one of Embodiments 341-379, wherein X⁴ is optionally substituted -CH═.
385. The oligonucleotide of any one of Embodiments 341-379, wherein X⁴ is —CH═.
386. The oligonucleotide of any one of Embodiments 341-379, wherein X⁴ is —CF═.
387. The oligonucleotide of any one of Embodiments 341-379, wherein X⁴ is —CCl═.
388. The oligonucleotide of any one of Embodiments 341-379, wherein X⁴ is —CBr═.
389. The oligonucleotide of any one of Embodiments 341-379, wherein X⁴ is —Cl═.
390. The oligonucleotide of any one of Embodiments 341-379, wherein X⁴ is —C(CH₃)═.
391. The oligonucleotide of any one of Embodiments 341-379, wherein X⁴ is —C(—N(R^{B4})₂)═.
392. The oligonucleotide of any one of Embodiments 341-379, wherein X⁴ is —C(—N(R^{B4})₂)=, wherein each R^{B4} is independently -L^{B4}-R^{B41}.
393. The oligonucleotide of any one of Embodiments 341-379, wherein X⁴ is optionally substituted -C(—NH₂)═.
394. The oligonucleotide of any one of Embodiments 341-379, wherein X⁴ is —C(—NH₂)═.
395. The oligonucleotide of any one of Embodiments 341-379, wherein X⁴ is —C(—N═CHNR₂)═.
396. The oligonucleotide of any one of Embodiments 341-379, wherein X⁴ is —C(—N═CHN(CH₃)₂)═.
397. The oligonucleotide of any one of Embodiments 341-379, wherein X⁴ is —C(—NHR′)═.
398. The oligonucleotide of any one of Embodiments 341-379, wherein X⁴ is —C(—NHC(O)R)═.
399. The oligonucleotide of any one of Embodiments 341-379, wherein X⁴ is —C(—NHAc)═.
400. The oligonucleotide of any one of Embodiments 341-379, wherein X⁴ is —C(R^{B4})₂—.
401. The oligonucleotide of any one of Embodiments 341-379, wherein X⁴ is optionally substituted —CH₂-.
402. The oligonucleotide of any one of Embodiments 341-379, wherein X⁴ is —CH₂-.
403. The oligonucleotide of any one of Embodiments 341-379, wherein X⁴ is optionally substituted —C(O)—.
404. The oligonucleotide of any one of Embodiments 341-379, wherein X⁴ is optionally substituted —C(S)—.
405. The oligonucleotide of any one of Embodiments 341-379, wherein X⁴ is optionally substituted —C(Se)-.
406. The oligonucleotide of any one of Embodiments 341-379, wherein X⁴ is optionally substituted —C(═NH)-.
407. The oligonucleotide of any one of Embodiments 341-379, wherein X⁴ is —C(═NR^{B4})-.
408. The oligonucleotide of any one of Embodiments 341-379, wherein X⁴ is —C(O)=, wherein the oxygen atom has a weaker hydrogen bond acceptor than the corresponding —C(O)- in U.
409. The oligonucleotide of any one of Embodiments 341-379, wherein X⁴ is —C(O)=, wherein the oxygen atom forms an intramolecular hydrogen bond.
410. The oligonucleotide of any one of Embodiments 341-379, wherein X⁴ is —C(O)=, wherein the oxygen atom forms an hydrogen bond with a hydrogen within the same nucleobase.
411. The oligonucleotide of any one of Embodiments 350-410, wherein X⁵ is —C(R^{B5})₂-.
412. The oligonucleotide of any one of Embodiments 350-410, wherein X⁵ is optionally substituted —CH₂-.
413. The oligonucleotide of any one of Embodiments 350-410, wherein X⁵ is —CH₂-.
414. The oligonucleotide of any one of Embodiments 350-410, wherein X⁵ is —C(R^{B5})═.
415. The oligonucleotide of any one of Embodiments 350-410, wherein X⁵ is —C(R^{B5})=, wherein R^{B5} is optionally substituted C₁₋₆ aliphatic.
416. The oligonucleotide of any one of Embodiments 350-410, wherein X⁵ is —C(R^{B5})=, wherein R^{B5} is C₁₋₆ aliphatic.
417. The oligonucleotide of any one of Embodiments 350-410, wherein X⁵ is optionally substituted -CH═.
418. The oligonucleotide of any one of Embodiments 350-410, wherein X⁵ is —CH═.
419. The oligonucleotide of any one of Embodiments 350-410, wherein X⁵ is —C(CH₃)-.
420. The oligonucleotide of any one of Embodiments 350-410, wherein X⁵ is —C(CH₂CH₃)-.
421. The oligonucleotide of any one of Embodiments 350-410, wherein X⁵ is —C(CH≡CH)═.
422. The oligonucleotide of any one of Embodiments 350-410, wherein X⁵ is —C(CH≡CCH₃)═.
423. The oligonucleotide of any one of Embodiments 350-410, wherein X⁵ is —CF=.
424. The oligonucleotide of any one of Embodiments 350-410, wherein X⁵ is —CCl=.
425. The oligonucleotide of any one of Embodiments 350-410, wherein X⁵ is —CBr=.
426. The oligonucleotide of any one of Embodiments 350-410, wherein X⁵ is —CI=.
427. The oligonucleotide of any one of Embodiments 350-410, wherein X⁵ is —C(CN)═.
428. The oligonucleotide of any one of Embodiments 350-410, wherein X⁵ is —C(NO₂)═.
429. The oligonucleotide of any one of Embodiments 350-410, wherein X⁵ is —C(-L^{B5}-R^{B51})═, wherein R^{B51} is -R′, —N(R′)₂, —OR′, or -SR′.
430. The oligonucleotide of any one of Embodiments 350-410, wherein X⁵ is —C(-L^{B5}-R^{B51})═, wherein R^{B51} is —N(R′)₂, —OR′, or -SR′.
431. The oligonucleotide of any one of Embodiments 350-410, wherein X⁵ is —C(-L^{B5}-R^{B51})=, wherein R^{B51} is —NHR′.
432. The oligonucleotide of any one of Embodiments 424-431, wherein L^{B5} is or comprises —C(O).
433. The oligonucleotide of any one of Embodiments 350-410, wherein X⁵ is —N═.
434. The oligonucleotide of any one of Embodiments 431-432, wherein X⁴ is —C(O)=, wherein the oxygen atom forms a hydrogen bond with a hydrogen of —NHR′, —OH or —SH in R^{B51}.
435. The oligonucleotide of any one of Embodiments 335-344, wherein Ring BA has the structure of formula BA-IV.
436. The oligonucleotide of any one of Embodiments 335-344, wherein Ring BA has the structure of formula BA-IV-a.
437. The oligonucleotide of any one of Embodiments 335-344, wherein Ring BA has the structure of formula BA-IV-b.
438. The oligonucleotide of any one of Embodiments 335-344, wherein Ring BA has the structure of formula BA-V.
439. The oligonucleotide of any one of Embodiments 335-344, wherein Ring BA has the structure of formula BA-V-a.
440. The oligonucleotide of any one of Embodiments 335-344, wherein Ring BA has the structure of formula BA-V-b. 441. The oligonucleotide of any one of Embodiments 335-344, wherein Ring BA has the structure of formula BA-VI. 442. The oligonucleotide of any one of Embodiments 435-441, wherein each of X, X², X³, X⁴, X⁵, X⁶, X”, X²′, X³′, X⁴′, X”, X”, and XT is independently and optionally substituted when it is —CH═, —C(OH)═, -C(—NH₂)═, —CH₂—, —C(═NH)—, or —NH—. 443. The oligonucleotide of any one of Embodiments 435-442, wherein X¹ is —N(-)-. 444. The oligonucleotide of any one of Embodiments 435-442, wherein X¹ is —C(-)═. 445. The oligonucleotide of any one of Embodiments 435-444, wherein X² is optionally substituted -CH═.
446. The oligonucleotide of any one of Embodiments 435-444, wherein X² is —CH═.
447. The oligonucleotide of any one of Embodiments 435-444, wherein X² is —N═.
448. The oligonucleotide of any one of Embodiments 435-444, wherein X² is —C(O)—.
449. The oligonucleotide of any one of Embodiments 435-444, wherein X² is —C(S)—.
450. The oligonucleotide of any one of Embodiments 435-444, wherein X² is —C(Se)-.
451. The oligonucleotide of any one of Embodiments 435-450, wherein X³ is —NR′-.
452. The oligonucleotide of any one of Embodiments 435-450, wherein X³ is optionally substituted -NH—.
453. The oligonucleotide of any one of Embodiments 435-450, wherein X³ is —NH—.
454. The oligonucleotide of any one of Embodiments 435-453, wherein Ring BA^{A} is 5-membered.
455. The oligonucleotide of any one of Embodiments 435-453, wherein Ring BA^{A} is 6-membered.
456. The oligonucleotide of any one of Embodiments 435-455, wherein Ring BA^{A} is 6-membered.
457. The oligonucleotide of any one of Embodiments 435-453, wherein Ring BA^{A} is 9-membered.
458. The oligonucleotide of any one of Embodiments 435-453, wherein Ring BA^{A} is 10-membered.
459. The oligonucleotide of any one of Embodiments 457-458, wherein Ring BA^{A} is bicyclic.
460. The oligonucleotide of any one of Embodiments 435-459, wherein Ring BA^{A} is an optionally substituted ring having 1-5 (e.g., 1, 2, 3, 4, or 5) heteroatoms.
461. The oligonucleotide of any one of Embodiments 435-460, wherein Ring BA^{A} is an optionally substituted ring having 1-3 heteroatoms.
462. The oligonucleotide of Embodiment 461, wherein a heteroatom is a nitrogen.
463. The oligonucleotide of any one of Embodiments 461-462, wherein Ring BA^{A} contains two nitrogen.
464. The oligonucleotide of any one of Embodiments 461-462, wherein a heteroatom is oxygen.
465. The oligonucleotide of any one of Embodiments 355-464, wherein X⁶ is —C(R^{B6})=, —C(OR^{B6})=, -C(R^{B6})₂—, or —C(O)—.
466. The oligonucleotide of any one of Embodiments 355-464, wherein X⁶ is —C(R)=, —C(R)₂—, or —C(O)—.
467. The oligonucleotide of any one of Embodiments 355-464, wherein X⁶ is optionally substituted -CH═.
468. The oligonucleotide of any one of Embodiments 355-464, wherein X⁶ is —CH═.
469. The oligonucleotide of any one of Embodiments 355-464, wherein X⁶ is —N═.
470. The oligonucleotide of any one of Embodiments 355-464, wherein X⁶ is optionally substituted —CH₂-.
471. The oligonucleotide of any one of Embodiments 355-464, wherein X⁶ is —CH₂-.
472. The oligonucleotide of any one of Embodiments 355-464, wherein X⁶ is —C(O)—.
473. The oligonucleotide of any one of Embodiments 355-464, wherein X⁶ is —C(S)—.
474. The oligonucleotide of any one of Embodiments 355-464, wherein X⁶ is —C(Se)-.
475. The oligonucleotide of any one of Embodiments 326-340, wherein Ring BA comprises [symbol] X⁴ [symbol] X^{5′} [symbol] .
476. The oligonucleotide of any one of Embodiments 335-340 or 475, wherein Ring BA has the structure of formula BA-VI.
477. The oligonucleotide of Embodiment 475, wherein X¹′ is —N(−)-.
478. The oligonucleotide of Embodiment 475, wherein X¹′ is —C(−)═.
479. The oligonucleotide of any one of Embodiments 475-478, wherein X²′ is —C(O)—.
480. The oligonucleotide of any one of Embodiments 475-478, wherein X²′ is —C(S)—.
481. The oligonucleotide of any one of Embodiments 475-478, wherein X²′ is —C(Se)-.
482. The oligonucleotide of any one of Embodiments 475-478, wherein X²′ is optionally substituted -CH═.
483. The oligonucleotide of any one of Embodiments 475-478, wherein X²′ is —CH═.
484. The oligonucleotide of any one of Embodiments 475-478, wherein X²′ is —C(R^{B2}′)═.
485. The oligonucleotide of any one of Embodiments 475-478, wherein X^{2′} is —C(OR^{B2}′)=, wherein R^{B2}′ is -L^{B2}′-R′.
486. The oligonucleotide of any one of Embodiments 475-478, wherein X²′ is —N═.
487. The oligonucleotide of any one of Embodiments 475-486, wherein X³′ is —NR′-.
488. The oligonucleotide of any one of Embodiments 475-486, wherein X³′ is optionally substituted -NH—.
489. The oligonucleotide of any one of Embodiments 475-486, wherein X^{3′} is —NH—.
490. The oligonucleotide of any one of Embodiments 475-486, wherein X³′ is —N(R^{B3}′)—, wherein R^{B3} is -L^{B3}′-R′.
491. The oligonucleotide of any one of Embodiments 475-486, wherein X³′ is —N(R′)-.
492. The oligonucleotide of any one of Embodiments 475-486, wherein X³′ is —N(C(O)R)-.
493. The oligonucleotide of any one of Embodiments 475-486, wherein X³′ is —C(R^{B3})═.
494. The oligonucleotide of any one of Embodiments 475-486, wherein X³′ is optionally substituted -CH═.
495. The oligonucleotide of any one of Embodiments 475-486, wherein X³′ is —CH═.
496. The oligonucleotide of any one of Embodiments 475-486, wherein X³′ is —N═.
497. The oligonucleotide of any one of Embodiments 475-496, wherein X⁴′ is —C(O)═.
498. The oligonucleotide of any one of Embodiments 475-496, wherein X⁴′ is —C(S)═.
499. The oligonucleotide of any one of Embodiments 475-496, wherein X⁴′ is —C(Se)═.
500. The oligonucleotide of any one of Embodiments 475-496, wherein X⁴′ is —C(R^{B4})═.
501. The oligonucleotide of any one of Embodiments 475-496, wherein X⁴′ is optionally substituted -CH═.
502. The oligonucleotide of any one of Embodiments 475-496, wherein X⁴′ is —CH═.
503. The oligonucleotide of any one of Embodiments 475-496, wherein X⁴′ is —C(OR^{B4}′)=, wherein R^{B4}′ is -L^{B4}′ R^{B41}.
504. The oligonucleotide of any one of Embodiments 475-496, wherein X⁴′ is —C(—N(R^{B4}′)₂)═.
505. The oligonucleotide of any one of Embodiments 475-496, wherein X⁴′ is —C(—N(R^{B4}′)₂)=, wherein each R^{B4} is independently -L^{B4}′R^{B41}′.
506. The oligonucleotide of any one of Embodiments 475-496, wherein X⁴′ is —C(—N(R^{B4}′)₂)═, wherein each R^{B4} is independently —H or —C(O)N(R′)₂.
507. The oligonucleotide of any one of Embodiments 475-496, wherein X⁴′ is —C(—N(R^{B4}′)₂)=, wherein each R^{B4} is independently —H or —C(O)NHR′.
508. The oligonucleotide of any one of Embodiments 475-496, wherein X⁴ is —C(—NHR^{B4})=, wherein R^{B4} is —C(O)N(R′)₂.
509. The oligonucleotide of any one of Embodiments 475-496, wherein X⁴′ is —C(—NHR^{B4}′)═, wherein R^{B4}′ is —C(O)NHR′.
510. The oligonucleotide of Embodiment 509, wherein R′ is optionally substituted phenyl.
511. The oligonucleotide of Embodiment 509, wherein R′ is optionally substituted naphthyl.
512. The oligonucleotide of Embodiment 509, wherein R′ is optionally substituted 2-naphthyl.
513. The oligonucleotide of Embodiment 509, wherein R′ is 2-naphthyl.
514. The oligonucleotide of Embodiment 509, wherein R′ is optionally substituted C₁₋₆ aliphatic.
515. The oligonucleotide of Embodiment 509, wherein R′ is methyl.
516. The oligonucleotide of any one of Embodiments 475-496, wherein X⁴ is —C(—N(R^{B4})₂)═, wherein each R^{B4}′ is independently R′.
517. The oligonucleotide of any one of Embodiments 475-496, wherein X⁴′ is —C(—N(R^{B4}′)₂)═, wherein each R^{B4} is independently —H or optionally substituted C₁₋₆ aliphatic.
518. The oligonucleotide of any one of Embodiments 475-496, wherein X⁴′ is optionally substituted -C(—NH₂)═.
519. The oligonucleotide of any one of Embodiments 475-496, wherein X⁴′ is —C(—NH₂)═.
520. The oligonucleotide of any one of Embodiments 475-496, wherein X⁴′ is —C(—NHEt)═.
521. The oligonucleotide of any one of Embodiments 475-496, wherein X⁴′ is —C(—NHiPr)═.
522. The oligonucleotide of any one of Embodiments 475-496, wherein X⁴′ is —C(—NHBz)═.
523. The oligonucleotide of any one of Embodiments 475-496, wherein X⁴′ is —C(—N(CH₃)₂)═.
524. The oligonucleotide of any one of Embodiments 475-496, wherein X⁴′ is —C(—N═CHN(CH₃)₂)═.
525. The oligonucleotide of any one of Embodiments 475-496, wherein X⁴′ is —C(—NHC(O)NHPh)═.
526. The oligonucleotide of any one of Embodiments 475-496, wherein X⁴′ is —C(—NHC(O)NHPh)═.
527. The oligonucleotide of any one of Embodiments 475-496, wherein X⁴′ is —C(R⁴)2-.
528. The oligonucleotide of any one of Embodiments 475-496, wherein X⁴′ is optionally substituted —CH₂—.
529. The oligonucleotide of any one of Embodiments 475-496, wherein X⁴′ is —C(═NR^{B4}′)=, wherein R^{B4} is -L^{B4}-R^{B41}′.
530. The oligonucleotide of any one of Embodiments 475-496, wherein X⁴ is —N(R^{B4}′), wherein R^{B4}′ is -L^{B4}′-R^{B41}′.
531. The oligonucleotide of any one of Embodiments 475-496, wherein X⁴′ is optionally substituted -NH—.
532. The oligonucleotide of any one of Embodiments 475-496, wherein X⁴′ is —NH—.
533. The oligonucleotide of any one of Embodiments 475-496, wherein X⁴′ is —N═.
534. The oligonucleotide of any one of Embodiments 475-533, wherein X^{5′} is optionally substituted -NH—.
535. The oligonucleotide of any one of Embodiments 475-533, wherein X^{5′} is —NH—.
536. The oligonucleotide of any one of Embodiments 475-533, wherein X^{5′} is optionally substituted —CH₂—.
537. The oligonucleotide of any one of Embodiments 475-533, wherein X^{5′} is —CH₂-.
538. The oligonucleotide of any one of Embodiments 475-533, wherein X^{5′} is —C(O)═.
539. The oligonucleotide of any one of Embodiments 475-533, wherein X^{5′} is —C(S)═.
540. The oligonucleotide of any one of Embodiments 475-533, wherein X⁵′ is —C(Se)═.
541. The oligonucleotide of any one of Embodiments 475-533, wherein X⁵′ is —N═.
542. The oligonucleotide of any one of Embodiments 475-533, wherein X⁵′ is —C(R^{B5}′).
543. The oligonucleotide of any one of Embodiments 475-533, wherein X^{5′} is optionally substituted -CH═.
544. The oligonucleotide of any one of Embodiments 475-533, wherein X⁵′ is —CH═.
545. The oligonucleotide of any one of Embodiments 475-544, wherein X^{6′} is —C(R^{B6}′)═.
546. The oligonucleotide of any one of Embodiments 475-544, wherein X⁶′ is optionally substituted —CH═.
547. The oligonucleotide of any one of Embodiments 475-544, wherein X^{6′} is —CH═.
548. The oligonucleotide of any one of Embodiments 475-544, wherein X⁶′ is —C(R^{B6}′)₂.
549. The oligonucleotide of any one of Embodiments 475-544, wherein X^{6′} is optionally substituted —CH₂-.
550. The oligonucleotide of any one of Embodiments 475-544, wherein X^{6′} is —C(O)═.
551. The oligonucleotide of any one of Embodiments 475-544, wherein X^{6′} is —C(S)═.
552. The oligonucleotide of any one of Embodiments 475-544, wherein X^{6′} is —C(Se)═.
553. The oligonucleotide of any one of Embodiments 475-544, wherein X^{6′} is —N═.
554. The oligonucleotide of any one of Embodiments 475-544, wherein X^{6′} is —C(—N(R^{B6}′)₂)=, wherein each R^{B6}′ is independently -L^{B6}′-B⁶¹′.
555. The oligonucleotide of any one of Embodiments 475-544, wherein X^{6′} is —C(—N(R′)₂)═.
556. The oligonucleotide of any one of Embodiments 475-544, wherein X⁶′ is —C(—NHR′)═.
557. The oligonucleotide of any one of Embodiments 475-544, wherein X^{6′} is —C(—NH₂)═.
558. The oligonucleotide of any one of Embodiments 475-544, wherein X⁶′ is —C(OR^{B6}′)═, wherein R^{B6}′ is -L^{B6}′-R^{B61}′.
559. The oligonucleotide of any one of Embodiments 475-544, wherein X⁶′ is —C(OR′)═.
560. The oligonucleotide of any one of Embodiments 475-559, wherein X⁷′ is —C(R^{B7}′).
561. The oligonucleotide of any one of Embodiments 475-559, wherein X⁷′ is optionally substituted —CH═.
562. The oligonucleotide of any one of Embodiments 475-559, wherein X⁷′ is —CH═.
563. The oligonucleotide of any one of Embodiments 475-559, wherein X⁷′ is —C(OR^{B7}′)═, wherein R^{B7}′ is -L^{B7}′-R^{B71}′.
564. The oligonucleotide of any one of Embodiments 475-559, wherein X⁷′ is —C(R^{B7}′)₂-.
565. The oligonucleotide of any one of Embodiments 475-559, wherein X⁷′ is optionally substituted —CH₂-.
566. The oligonucleotide of any one of Embodiments 475-559, wherein X⁷′ is —C(—N(R^{B7}′)₂)═. wherein each R^{B7}′ is independently -L^{B7}′- R^{B71}′.
567. The oligonucleotide of any one of Embodiments 475-559, wherein X⁷′ is —C(—N(R′)₂)═.
568. The oligonucleotide of any one of Embodiments 475-559, wherein X⁷′ is —C(—NH₂)═.
569. The oligonucleotide of any one of Embodiments 475-559, wherein X⁷′ is —C(—NHR′)═.
570. The oligonucleotide of any one of Embodiments 475-559, wherein X⁷′ is —C(—NHC(O)R)═.
571. The oligonucleotide of any one of Embodiments 475-559, wherein X⁷′ is —C(—NHC(O)CH₃)═.
572. The oligonucleotide of any one of Embodiments 475-559, wherein X⁷′ is —N(R^{B7}′)-, wherein each R^{B7}′ is independently -L^{B7}′-R^{B71}′.
573. The oligonucleotide of any one of Embodiments 475-559, wherein X⁷′ is optionally substituted -NH—.
574. The oligonucleotide of any one of Embodiments 475-559, wherein X⁷′ is —NH—.
575. The oligonucleotide of any one of Embodiments 475-559, wherein X⁷′ is —N═.
576. The oligonucleotide of any one of Embodiments 475-575, wherein X⁸′ is C.
577. The oligonucleotide of any one of Embodiments 475-575, wherein X⁸′ is N.
578. The oligonucleotide of any one of Embodiments 475-577, wherein X⁹′ is C.
579. The oligonucleotide of any one of Embodiments 475-577, wherein X^{9′} is N.
580. The oligonucleotide of any one of Embodiments 335-340, wherein Ring BA is

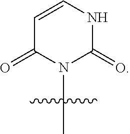 [SMILES: *n1c(=O)cc[nH]c1=O]

581. The oligonucleotide of any one of Embodiments 335-340, wherein Ring BA is

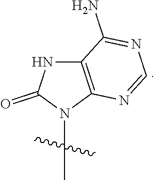 [SMILES: *n1c(=O)[nH]c2c(N)ncnc21]

582. The oligonucleotide of any one of Embodiments 335-340, wherein Ring BA is

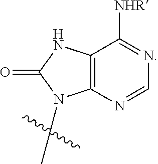

583. The oligonucleotide of any one of Embodiments 335-340, wherein Ring BA is

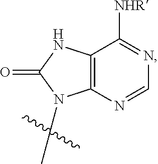

wherein R′ is —C(O)R.
584. The oligonucleotide of any one of Embodiments 335-340, wherein Ring BA is

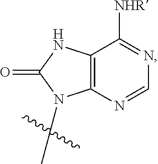

wherein R′ is —C(O)Ph.
585. The oligonucleotide of any one of Embodiments 335-340, wherein Ring BA is

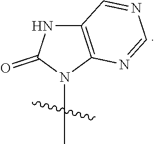 [SMILES: *n1c(=O)[nH]c2cncnc21]

586. The oligonucleotide of any one of Embodiments 335-340, wherein Ring BA is

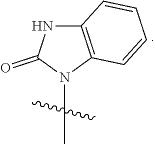 [SMILES: *n1c(=O)[nH]c2ccccc21]

587. The oligonucleotide of any one of Embodiments 335-340, wherein Ring BA is

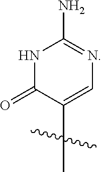 [SMILES: *c1cnc(N)[nH]c1=O]

588. The oligonucleotide of any one of Embodiments 335-340, wherein Ring BA is

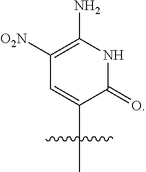 [SMILES: *c1cc([N+](=O)[O-])c(N)[nH]c1=O]

589. The oligonucleotide of any one of Embodiments 335-340, wherein Ring BA is

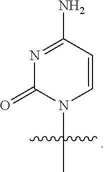 [SMILES: *n1ccc(N)nc1=O]

590. The oligonucleotide of any one of Embodiments 335-340, wherein Ring BA is

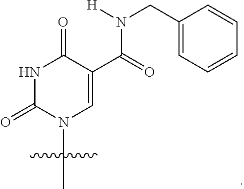 [SMILES: *n1cc(C(=O)NCc2ccccc2)c(=O)[nH]c1=O]

591. The oligonucleotide of any one of Embodiments 335-340, wherein Ring BA is

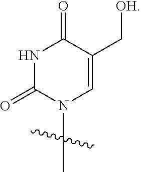 [SMILES: *n1cc(CO)c(=O)[nH]c1=O]

592. The oligonucleotide of any one of Embodiments 335-340, wherein Ring BA is

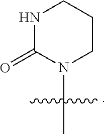 [SMILES: *N1CCCNC1=O]

593. The oligonucleotide of any one of Embodiments 335-340, wherein Ring BA is

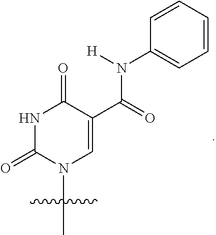 [SMILES: *n1cc(C(=O)Nc2ccccc2)c(=O)[nH]c1=O]

594. The oligonucleotide of any one of Embodiments 335-340, wherein Ring BA is

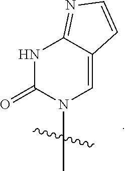 [SMILES: *n1cc2ccnc-2[nH]c1=O]

595. The oligonucleotide of any one of Embodiments 335-340, wherein Ring BA is

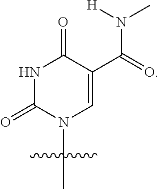 [SMILES: *n1cc(C(=O)NC)c(=O)[nH]c1=O]

596. The oligonucleotide of any one of Embodiments 335-340, wherein Ring BA is

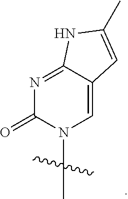 [SMILES: *n1cc2cc(C)[nH]c2nc1=O]

597. The oligonucleotide of any one of Embodiments 335-340, wherein Ring BA is

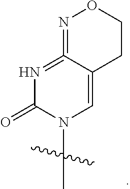

598. The oligonucleotide of any one of Embodiments 335-340, wherein Ring BA is

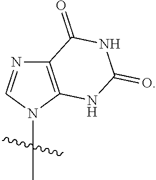 [SMILES: *n1cnc2c(=O)[nH]c(=O)[nH]c21]

599. The oligonucleotide of any one of Embodiments 335-340, wherein Ring BA is

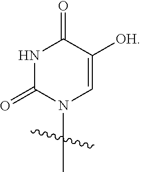 [SMILES: *n1cc(O)c(=O)[nH]c1=O]

600. The oligonucleotide of any one of Embodiments 335-340, wherein Ring BA is

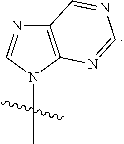 [SMILES: *n1cnc2cncnc21]

601. The oligonucleotide of any one of Embodiments 335-340, wherein Ring BA is

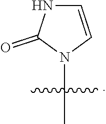 [SMILES: *n1cc[nH]c1=O]

602. The oligonucleotide of any one of Embodiments 335-340, wherein Ring BA is

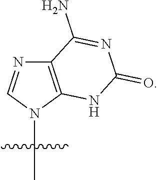 [SMILES: *n1cnc2c(N)nc(=O)[nH]c21]

603. The oligonucleotide of any one of Embodiments 335-340, wherein Ring BA is

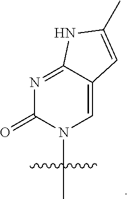 [SMILES: *n1cc2cc(C)[nH]c2nc1=O]

604. The oligonucleotide of any one of Embodiments 335-340, wherein Ring BA is

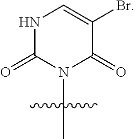 [SMILES: *n1c(=O)[nH]cc(Br)c1=O]

605. The olignucleotide of any one of Embodiments 35-40,wherein Ring BA is

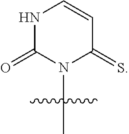 [SMILES: *n1c(=S)cc[nH]c1=O]

606. The oligonucleotide of any one of Embodiments 335-340, wherein Ring BA is

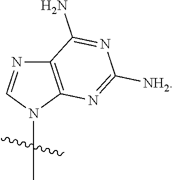 [SMILES: *n1cnc2c(N)nc(N)nc21]

607. The oligonucleotide of any one of Embodiments 335-340, wherein Ring BA is

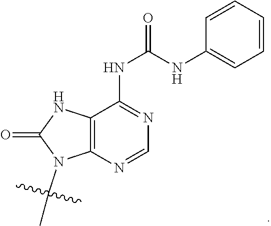 [SMILES: *n1c(=O)[nH]c2c(NC(=O)Nc3ccccc3)ncnc21]

608. The oligonucleotide of any one of Embodiments 335-340, wherein Ring BA is

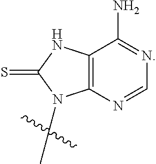 [SMILES: *n1c(=S)[nH]c2c(N)ncnc21]

609. The oligonucleotide of any one of Embodiments 335-340, wherein Ring BA is

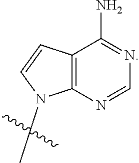 [SMILES: *n1ccc2c(N)ncnc21]

610. The oligonucleotide of any one of Embodiments 335-340, wherein Ring BA is

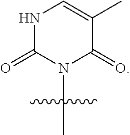 [SMILES: *n1c(=O)[nH]cc(C)c1=O]

611. The oligonucleotide of any one of Embodiments 335-340, wherein Ring BA is

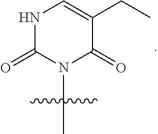 [SMILES: *n1c(=O)[nH]cc(CC)c1=O]

612. The oligonucleotide of any one of Embodiments 335-340, wherein Ring BA is

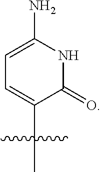 [SMILES: *c1ccc(N)[nH]c1=O]

613. The oligonucleotide of any one of Embodiments 335-340, wherein Ring BA is

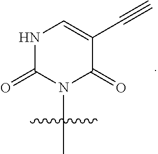 [SMILES: *n1c(=O)[nH]cc(C#C)c1=O]

614. The oligonucleotide of any one of Embodiments 335-340, wherein Ring BA is

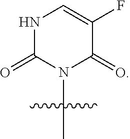 [SMILES: *n1c(=O)[nH]cc(F)c1=O]

615. The oligonucleotide of any one of Embodiments 335-340, wherein Ring BA is

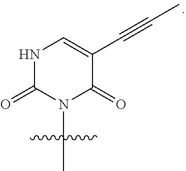 [SMILES: *n1c(=O)[nH]cc(C#CC)c1=O]

616. The oligonucleotide of any one of Embodiments 335-340, wherein Ring BA is

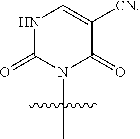 [SMILES: *n1c(=O)[nH]cc(C#N)c1=O]

617. The oligonucleotide of any one of Embodiments 335-340, wherein Ring BA is,

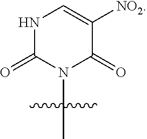 [SMILES: *n1c(=O)[nH]cc([N+](=O)[O-])c1=O]

618. The oligonucleotide of any one of Embodiments 335-340, wherein Ring BA is

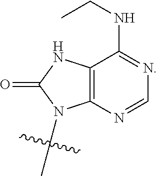 [SMILES: *n1c(=O)[nH]c2c(NCC)ncnc21]

619. The oligonucleotide of any one of Embodiments 335-340, wherein Ring BA is

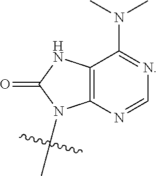 [SMILES: *n1c(=O)[nH]c2c(N(C)C)ncnc21]

620. The oligonucleotide of any one of Embodiments 335-340, wherein Ring BA is

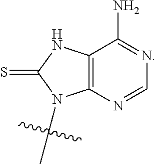 [SMILES: *n1c(=S)[nH]c2c(N)ncnc21]

621. The oligonucleotide of any one of Embodiments 335-340, wherein Ring BA is

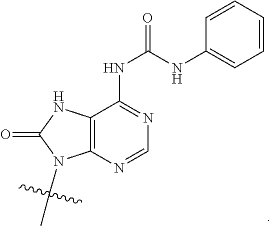 [SMILES: *n1c(=O)[nH]c2c(NC(=O)Nc3ccccc3)ncnc21]

622. The oligonucleotide of any one of Embodiments 335-340, wherein Ring BA is

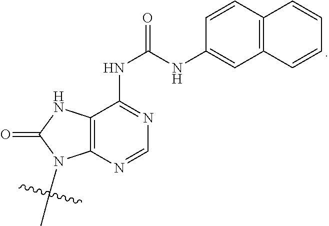 [SMILES: *n1c(=O)[nH]c2c(NC(=O)Nc3ccc4ccccc4c3)ncnc21]

623. The oligonucleotide of any one of Embodiments 335-340, wherein Ring BA is

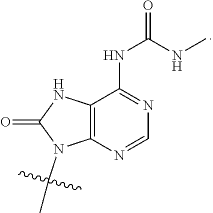 [SMILES: *n1c(=O)[nH]c2c(NC(=O)NC)ncnc21]

624. The oligonucleotide of any one of Embodiments 335-340, wherein Ring BA is

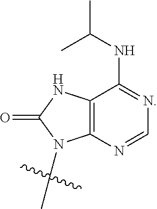 [SMILES: *n1c(=O)[nH]c2c(NC(C)C)ncnc21]

625. The oligonucleotide of any one of Embodiments 335-340, wherein Ring BA is

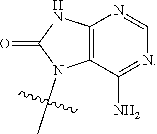 [SMILES: *n1c(=O)[nH]c2ncnc(N)c21]

626. The oligonucleotide of any one of Embodiments 335-340, wherein Ring BA is

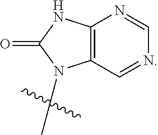 [SMILES: *n1c(=O)[nH]c2ncncc21]

627. The oligonucleotide of any one of Embodiments 335-340, wherein Ring BA is

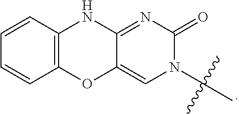 [SMILES: *n1cc2c(nc1=O)Nc1ccccc1O2]

628. The oligonucleotide of any one of Embodiments 335-340, wherein Ring BA is

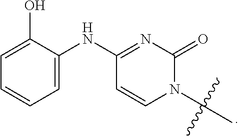 [SMILES: *n1ccc(Nc2ccccc2O)nc1=O]

629. The oligonucleotide of any one of Embodiments 335-628, wherein a nucleobase is Ring BA or a tautomer thereof.
630. The oligonucleotide of any one of Embodiments 335-628, wherein a nucleobase is substituted Ring BA or a tautomer thereof.
631. The oligonucleotide of any one of Embodiments 335-628, wherein a nucleobase is optionally substituted Ring BA or a tautomer thereof, wherein each ring —CH═, —CH₂— and —NH— is optionally and independently substituted.
632. The oligonucleotide of any one of Embodiments 335-628, wherein a nucleobase is optionally substituted Ring BA or a tautomer thereof, wherein each ring —CH= and —CH₂— is optionally and independently substituted.
633. The oligonucleotide of any one of Embodiments 335-628, wherein a nucleobase is optionally substituted Ring BA or a tautomer thereof, wherein each ring —CH═ is optionally and independently substituted.
634. The oligonucleotide of any one of the preceding Embodiments, comprising a sugar that comprises a 6-membered ring having 0-2 (0, 1, or 2) heteroatoms.
635. The oligonucleotide of any one of the preceding Embodiments, comprising a bicyclic sugar.
636. The oligonucleotide of any one of the preceding Embodiments, comprising a LNA sugar.
637. The oligonucleotide of any one of the preceding Embodiments, comprising a cEt sugar.
638. The oligonucleotide of any one of the preceding Embodiments, comprising a (S)-cEt sugar.
639. The oligonucleotide of any one of the preceding Embodiments, comprising a sugar that has the structure of

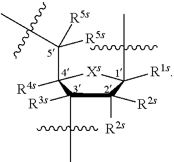

640. The oligonucleotide of Embodiment 639, wherein each R^{2s} is independently —H, —F, —OH or —OR, wherein R^{ak} is optionally substituted C₁₋₆ aliphatic.
641. The oligonucleotide of any one of Embodiments 639-640, wherein the sugar has the structure of

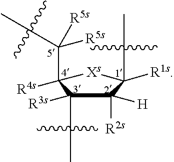

642. The oligonucleotide of any one of Embodiments 639-641, wherein the sugar has the structure of

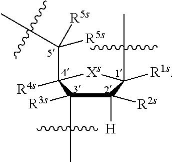

643. The oligonucleotide of any one of Embodiments 639-642, wherein R^{1s} is —H.
644. The oligonucleotide of any one of Embodiments 639-643, wherein R^{2s} is —H.
645. The oligonucleotide of any one of Embodiments 639-643, wherein R^{2s} is -F.
646. The oligonucleotide of any one of Embodiments 639-643, wherein R^{2s} is —OH.
647. The oligonucleotide of any one of Embodiments 639-643, wherein R^{2s} is —OR^{ak}, wherein R^{ak} is optionally substituted C₁₋₆ aliphatic.
648. The oligonucleotide of any one of Embodiments 639-643, wherein R^{2a} is —OMe.
649. The oligonucleotide of any one of Embodiments 639-643, wherein R^{2s} is —OCH₂CH₂OCH₃.
650. The oligonucleotide of any one of Embodiments 639-649, wherein R^{3s} is —H.
651. The oligonucleotide of any one of Embodiments 639-650, wherein R^{4s} is —H.
652. The oligonucleotide of any one of Embodiments 639-651, wherein one occurrence of R^{5s} is —H.
653. The oligonucleotide of any one of Embodiments 639-652, wherein the other occurrence of R^{5s} is —H.
654. The oligonucleotide of any one of Embodiments 639-652, wherein the other occurrence of R^{5s} is optionally substituted C₁₋₆ aliphatic.
655. The oligonucleotide of any one of the preceding Embodiments, comprising a sugar that is optionally substituted

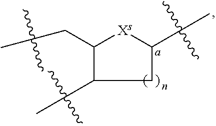

wherein position a is bonded to a nucleobase, wherein X^{s} is —O—, —S— or —Se—, and n is 0, 1, 2or 3.
656. The oligonucleotide of any one of the preceding Embodiments, comprising a sugar that is

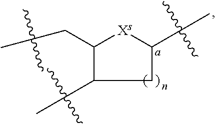

wherein position a is bonded to a nucleobase, wherein X^{s} is —O—, —S— or —Se—. and n is 0, 1,2 or 3.
657. The oligonucleotide of any one of the preceding Embodiments, comprising a sugar that is optionally substituted

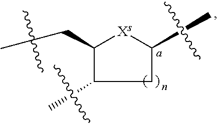

wherein position a is bonded to a nucleobase, wherein X^{s} is -O—, —S— or —Se—, and n is 0, 1, 2 or 3.
658. The oligonucleotide of any one of the preceding Embodiments, comprising a sugar that is

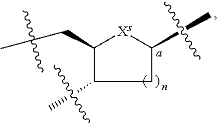

wherein position a is bonded to a nucleobase, wherein X^{s} is —O—, —S— or —Se—, and n is 0, 1,2 or 3.
659. The oligonucleotide of any one of Embodiments 655-658, wherein n is 0.
660. The oligonucleotide of any one of Embodiments 655-658, wherein n is 1.
661. The oligonucleotide of any one of Embodiments 655-658, wherein n is 2.
662. The oligonucleotide of any one of Embodiments 655-658, wherein n is 3.
663. The oligonucleotide of any one of Embodiments 639-662, wherein X^{s} is —O—.
664. The oligonucleotide of any one of Embodiments 639-662, wherein X^{s} is -S-.
665. The oligonucleotide of any one of Embodiments 639-662, wherein X^{s} is optionally substituted —CH₂—.
666. The oligonucleotide of any one of Embodiments 639-662, wherein X^{s} is —CH₂-.
667. The oligonucleotide of any one of the preceding Embodiments, wherein the second domain comprises about 1-50 (e.g., about 5, 6, 7, 8, 9, or 10-about 10, 11, 12, 13, 14, 15, 16. 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
668. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85% 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40% 50%, 60%, 65%, 70%, 75%, 80%, 85%, 90%, 95%, or 100%, etc.) of sugars in the second domain are independently modified sugars with a modification that is not 2′-F.
669. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the second domain are independently modified sugars with a modification that is not 2′-F.
670. The oligonucleotide of any one of Embodiments 331-669, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
671. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
672. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
673. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
674. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
675. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
676. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
677. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
678. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
679. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
680. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
681. The oligonucleotide of any one of the preceding Embodiments, wherein the second domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
682. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the second domain are modified internucleotidic linkages.
683. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the second domain are modified internucleotidic linkages.
684. The oligonucleotide of any one of the preceding Embodiments, wherein each modified internucleotidic linkages is independently a chiral internucleotidic linkage.
685. The oligonucleotide of any one of the preceding Embodiments, wherein each modified internucleotidic linkages is independently a phosphorothioate internucleotidic linkage or a non-negatively charged internucleotidic linkage.
686. The oligonucleotide of any one of the preceding Embodiments, wherein each modified internucleotidic linkages is independently a phosphorothioate internucleotidic linkage or a neutral internucleotidic linkage.
687. The oligonucleotide of any one of the preceding Embodiments, wherein the second domain comprises one or more phosphorothioate internucleotidic linkages.
688. The oligonucleotide of any one of the preceding Embodiments, wherein the second domain comprises 1, 2, 3, 4, or 5 non-negatively charged internucleotidic linkages.
689. The oligonucleotide of any one of the preceding Embodiments, wherein the internucleotidic linkage between the last and the second last nucleosides of the second domain is a non-negatively charged internucleotidic linkage.
690. The oligonucleotide of any one of the preceding Embodiments, wherein the internucleotidic linkage between the first and the second nucleosides of the second domain is a non-negatively charged internucleotidic linkage.
691. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second domain is chirally controlled.
692. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second domain is chirally controlled.
693. The oligonucleotide of any one of the preceding Embodiments, wherein the internucleotidic linkage between the last and the second last nucleosides of the second domain is chirally controlled.
694. The oligonucleotide of any one of the preceding Embodiments, wherein the internucleotidic linkage between the first and the second nucleosides of the second domain is a chirally controlled.
695. The oligonucleotide of any one of the preceding Embodiments, wherein each chiral internucleotidic linkage is independently a chirally controlled internucleotidic linkage.
696. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second domain is Sp.
697. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second domain is Sp, or wherein each chiral internucleotidic linkages in the second domain is Sp.
698. The oligonucleotide of any one of the preceding Embodiments, wherein the internucleotidic linkage between the first and the second nucleosides of the second domain is Rp.
699. The oligonucleotide of any one of the preceding Embodiments, wherein the internucleotidic linkage between the last and the second last nucleosides of the second domain is Rp.
700. The oligonucleotide of any one of the preceding Embodiments, wherein each internucleotidic linkage in the second domain is independently a modified internucleotidic linkage.
701. The oligonucleotide of any one of Embodiments 1-699, wherein the second domain comprises one or more natural phosphate linkages.
702. The oligonucleotide of any one of the preceding Embodiments, wherein the second domain can recruit, or promotes or contributes to recruitment of, an ADAR protein to a target nucleic acid.
703. The oligonucleotide of any one of the preceding Embodiments, wherein the second domain can interact, or promotes or contributes to interaction of, an ADAR protein with a target nucleic acid.
704. The oligonucleotide of any one of the preceding Embodiments, wherein the second domain contacts with a domain that have an enzymatic activity.
705. The oligonucleotide of any one of the preceding Embodiments, wherein the second domain contact with a domain that has a deaminase activity of ADAR1.
706. The oligonucleotide of any one of the preceding Embodiments, wherein the second domain contact with a domain that has a deaminase activity of ADAR2.
707. The oligonucleotide of any one of the preceding Embodiments, wherein the second domain comprises or consists of from the 5′ to 3′ a first subdomain, a second subdomain, and a third subdomain.
708. The oligonucleotide of any one of the preceding Embodiments, wherein the second domain consists of from the 5′ to 3′ a first subdomain, a second subdomain, and a third subdomain.
709. The oligonucleotide of any one of the preceding Embodiments, wherein the first subdomain has a length of about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
710. The oligonucleotide of any one of the preceding Embodiments, wherein the first subdomain has a length of about 10-20 (e.g., about 10, 11, 12, 13, 14, 15, 16, 17, 18, 19, or 20) nucleobases.
711. The oligonucleotide of any one of the preceding Embodiments, wherein the first subdomain comprises one or more (e.g., 1-10, 1, 2, 3, 4, 5, 6, 7, 8, 9, or 10, etc.) mismatches when the oligonucleotide is aligned with a target nucleic acid for complementarity.
712. The oligonucleotide of any one of the preceding Embodiments, wherein the first subdomain comprises two or more mismatches when the oligonucleotide is aligned with a target nucleic acid for complementarity.
713. The oligonucleotide of any one of Embodiments 1-711, wherein the first subdomain comprises one and no more than one mismatch when the oligonucleotide is aligned with a target nucleic acid for complementarity.
714. The oligonucleotide of any one of Embodiments 1-711, wherein the first subdomain comprises two and no more than two mismatches when the oligonucleotide is aligned with a target nucleic acid for complementarity.
715. The oligonucleotide of any one of the preceding Embodiments, wherein the first subdomain comprises one or more (e.g., 1-10, 1, 2, 3, 4, 5, 6, 7, 8, 9, or 10, etc.) bulges when the oligonucleotide is aligned with a target nucleic acid for complementarity.
716. The oligonucleotide of Embodiment 715, wherein each bulge independently comprises one or more base pairs that are not Watson-Crick or wobble pairs.
717. The oligonucleotide of any one of the preceding Embodiments, wherein the first subdomain comprises one or more (e.g., 1-10, 1, 2, 3, 4, 5, 6, 7, 8, 9, or 10, etc.) wobble pairs when the oligonucleotide is aligned with a target nucleic acid for complementarity.
718. The oligonucleotide of any one of the preceding Embodiments, wherein the first subdomain comprises two or more wobble pairs when the oligonucleotide is aligned with a target nucleic acid for complementarity.
719. The oligonucleotide of any one of the preceding Embodiments, wherein the first subdomain comprises two and no more than two wobble pairs when the oligonucleotide is aligned with a target nucleic acid for complementarity.
720. The oligonucleotide of any one of Embodiments 1-709, wherein the first subdomain is fully complementary to a target nucleic acid.
721. The oligonucleotide of any one of the preceding Embodiments, wherein the first subdomain comprises about 1-50 (e.g., about 5, 6, 7, 8, 9, or 10-about 10, 11, 12, 13, 14, 15, 16. 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
722. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40% 50%, 60%, 65%, 70%, 75%, 80%, 85%, 90%, 95%, or 100%, etc.) of sugars in the first subdomain are independently modified sugars with a modification that is not 2′-F.
723. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first subdomain are independently modified sugars with a modification that is not 2′-F.
724. The oligonucleotide of any one of Embodiments 721-723, wherein the first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
725. The oligonucleotide of any one of Embodiments 721-723, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
726. The oligonucleotide of any one of Embodiments 721-723, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
727. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
728. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
729. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
730. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
731. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
732. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
733. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
734. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
735. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
736. The oligonucleotide of any one of Embodiments 707-726, wherein each sugar in the first subdomain independently comprises a 2′-OR modification, wherein R is optionally substituted C₁₋₆ aliphatic, or a 2′-O-L^{B}-4′ modification.
737. The oligonucleotide of Embodiment 736, wherein each sugar in the first subdomain independently comprises a 2′-OR modification, wherein R is optionally substituted C₁₋₆ aliphatic, or a 2′-O-L^{B}-4′ modification, wherein L^{B} is optionally substituted —CH₂-.
738. The oligonucleotide of Embodiment 736, wherein each sugar in the first subdomain independently comprises 2′-OMe.
739. The oligonucleotide of any one of the preceding Embodiments, wherein the first subdomain comprises a 5′-end portion having a length of about 3-8 nucleobases.
740. The oligonucleotide of Embodiment 739, wherein the 5′-end portion has a length of about 3-6 nucleobases.
741. The oligonucleotide of Embodiment 739 or 740, wherein the 5′-end portion comprises the 5′-end nucleobase of the first subdomain.
742. The oligonucleotide of any one of Embodiments 739-741, wherein one or more of the sugars in the 5′-end portion are independently modified sugars.
743. The oligonucleotide of Embodiment 742, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
744. The oligonucleotide of Embodiment 742, wherein one or more of the modified sugars independently comprises 2′-F or 2′-OR, wherein R is independently optionally substituted C₁₋₆ aliphatic.
745. The oligonucleotide of Embodiment 742, wherein one or more of the modified sugars are independently 2′-F or 2′-OMe.
746. The oligonucleotide of any one of Embodiments 739-745, wherein the 5′-end portion comprises one or more mismatches.
747. The oligonucleotide of any one of Embodiments 739-746, wherein the 5′-end portion comprises one or more wobbles.
748. The oligonucleotide of any one of Embodiments 739-747, wherein the 5′-end portion is about 60-100% (e.g., 66%, 70%, 75%, 80%, 85%, 90%, 95%, or more) complementary to a target nucleic acid.
749. The oligonucleotide of any one of the preceding Embodiments, wherein the first subdomain comprises a 3′-end portion having a length of about 3-8 nucleobases.
750. The oligonucleotide of Embodiment 749, wherein the 3′-end portion has a length of about 1-3 nucleobases.
751. The oligonucleotide of Embodiment 749 or 750, wherein the 3′-end portion comprises the 3′-end nucleobase of the first subdomain.
752. The oligonucleotide of any one of Embodiments 749-751, wherein one or more of the sugars in the 3′-end portion are independently modified sugars.
753. The oligonucleotide of Embodiment 752, wherein the modified sugars are independently selected from a bicyclic sugar (e.g., a LNA sugar), an acyclic sugar (e.g.. a UNA sugar), a sugar with a 2′-OR modification, or a sugar with a 2′-N(R)₂ modification, wherein each R is independently optionally substituted C₁₋₆ aliphatic.
754. The oligonucleotide of Embodiment 752, wherein one or more of the modified sugars independently comprise 2′-F.
755. The oligonucleotide of any one of Embodiments 752-754, wherein no modified sugars comprise 2′-OMe.
756. The oligonucleotide of any one of Embodiments 749-755, wherein each sugar of the 3′-end portion independently comprises two 2′-H or a 2′-F modification.
757. The oligonucleotide of any one of Embodiments 739-745, wherein the 3′-end portion comprises one or more mismatches.
758. The oligonucleotide of any one of Embodiments 739-746, wherein the 3′-end portion comprises one or more wobbles.
759. The oligonucleotide of any one of Embodiments 739-747, wherein the 3′-end portion is about 60-100% (e.g., 66%, 70%, 75%, 80%, 85%, 90%, 95%, or more) complementary to a target nucleic acid.
760. The oligonucleotide of any one of the preceding Embodiments, wherein the first subdomain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
761. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the first subdomain are modified internucleotidic linkages.
762. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the first subdomain are modified internucleotidic linkages.
763. The oligonucleotide of any one of the preceding Embodiments, wherein each modified internucleotidic linkages is independently a chiral internucleotidic linkage.
764. The oligonucleotide of any one of the preceding Embodiments, wherein the internucleotidic linkage between the first and the second nucleosides of the first subdomain is a non-negatively charged internucleotidic linkage.
765. The oligonucleotide of any one of the preceding Embodiments, wherein each modified internucleotidic linkages is independently a phosphorothioate internucleotidic linkage or a non-negatively charged internucleotidic linkage.
766. The oligonucleotide of any one of the preceding Embodiments, wherein each modified internucleotidic linkages is independently a phosphorothioate internucleotidic linkage or a neutral internucleotidic linkage.
767. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10. 11, 12, 13, 14, 15, 16, 17, 18, 19, or 20, etc.) chiral internucleotidic linkages in the first subdomain is chirally controlled.
768. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95% or 100%, etc.) of chiral internucleotidic linkages in the first subdomain is chirally controlled.
769. The oligonucleotide of any one of the preceding Embodiments, wherein the internucleotidic linkage between the first and the second nucleosides of the first subdomain is chirally controlled.
770. The oligonucleotide of any one of the preceding Embodiments, wherein each chiral internucleotidic linkage is independently a chirally controlled internucleotidic linkage.
771. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first subdomain is Sp.
772. The oligonucleotide of any one of the preceding Embodiments, wherein at least 5%-100% (e.g.. about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first subdomain is Sp.
773. The oligonucleotide of any one of the preceding Embodiments, wherein each chiral internucleotidic linkages in the first subdomain is Sp.
774. The oligonucleotide of any one of Embodiments 1-773, wherein the internucleotidic linkage between the first and the second nucleosides of the first subdomain is Rp.
775. The oligonucleotide of any one of the preceding Embodiments, wherein each internucleotidic linkage in the first subdomain is independently a modified internucleotidic linkage.
776. The oligonucleotide of any one of Embodiments 1-774, wherein the first subdomain comprises one or more natural phosphate linkages.
777. The oligonucleotide of any one of the preceding Embodiments, wherein the first subdomain can recruit, or promotes or contributes to recruitment of, an ADAR protein to a target nucleic acid.
778. The oligonucleotide of any one of the preceding Embodiments, wherein the first subdomain can interact, or promotes or contributes to interaction of. an ADAR protein with a target nucleic acid.
779. The oligonucleotide of any one of the preceding Embodiments, wherein the first subdomain contacts with a domain that have an enzymatic activity.
780. The oligonucleotide of any one of the preceding Embodiments, wherein the first subdomain contact with a domain that has a deaminase activity of ADAR1.
781. The oligonucleotide of any one of the preceding Embodiments, wherein the first subdomain contact with a domain that has a deaminase activity of ADAR2.
782. The oligonucleotide of any one of the preceding Embodiments, wherein the second subdomain has a length of about 1-10 (e.g., about 1, 2, 3, 4, 5, 6, 7, 8, 9, or 10) nucleobases.
783. The oligonucleotide of any one of the preceding Embodiments, wherein the second subdomain has a length of about 1-5 (e.g., about 1, 2, 3, 4, or 5) nucleobases.
784. The oligonucleotide of any one of the preceding Embodiments, wherein the second subdomain has a length of about 1, 2, or 3 nucleobases.
785. The oligonucleotide of any one of the preceding Embodiments, wherein the second subdomain has a length of 3 nucleobases.
786. The oligonucleotide of any one of the preceding Embodiments, wherein the second subdomain comprises a nucleoside opposite to a target adenosine.
787. The oligonucleotide of any one of the preceding Embodiments, wherein the second domain comprises one and no more than one nucleoside opposite to a target adenosine.
788. The oligonucleotide of any one of the preceding Embodiments, wherein the second subdomain comprises one or more (e.g., 1-10, 1, 2, 3, 4, 5, 6, 7, 8, 9, or 10, etc.) mismatches when the oligonucleotide is aligned with a target nucleic acid for complementarity.
789. The oligonucleotide of any one of the preceding Embodiments, wherein the second subdomain comprises two or more mismatches when the oligonucleotide is aligned with a target nucleic acid for complementarity.
790. The oligonucleotide of any one of Embodiments 1-788, wherein the second subdomain comprises one and no more than one mismatch when the oligonucleotide is aligned with a target nucleic acid for complementarity.
791. The oligonucleotide of any one of Embodiments 1-788, wherein the second subdomain comprises two and no more than two mismatches when the oligonucleotide is aligned with a target nucleic acid for complementarity.
792. The oligonucleotide of any one of the preceding Embodiments, wherein the second subdomain comprises one or more (e.g., 1-10, 1, 2, 3, 4, 5, 6, 7, 8, 9, or 10, etc.) bulges when the oligonucleotide is aligned with a target nucleic acid for complementarity.
793. The oligonucleotide of Embodiment 792, wherein each bulge independently comprises one or more base pairs that are not Watson-Crick or wobble pairs.
794. The oligonucleotide of any one of the preceding Embodiments, wherein the second subdomain comprises one or more (e.g., 1-10, 1, 2, 3, 4, 5, 6, 7, 8, 9, or 10, etc.) wobble pairs when the oligonucleotide is aligned with a target nucleic acid for complementarity.
795. The oligonucleotide of any one of the preceding Embodiments, wherein the second subdomain comprises two or more wobble pairs when the oligonucleotide is aligned with a target nucleic acid for complementarity.
796. The oligonucleotide of any one of the preceding Embodiments, wherein the second subdomain comprises two and no more than two wobble pairs when the oligonucleotide is aligned with a target nucleic acid for complementarity.
797. The oligonucleotide of any one of Embodiments 1-787, wherein the second subdomain is fully complementary to a target nucleic acid.
798. The oligonucleotide of any one of the preceding Embodiments, wherein the second subdomain comprises one or more sugars comprising two 2′-H (e.g., natural DNA sugars).
799. The oligonucleotide of any one of the preceding Embodiments, wherein the second subdomain comprises one or more sugars comprising 2′-OH (e.g., natural RNA sugars).
800. The oligonucleotide of any one of the preceding Embodiments, wherein the second subdomain comprises about 1-10 (e.g., 1, 2, 3, 4, 5, 6, 7, 8, 9, or 10) modified sugars.
801. The oligonucleotide of Embodiment 800, wherein each modified sugar is independently selected from a bicyclic sugar (e.g., a LNA sugar), an acyclic sugar (e.g.. a UNA sugar), a sugar with a 2′-OR modification, or a sugar with a 2′-N(R)₂ modification, wherein each R is independently optionally substituted C₁₋₆ aliphatic.
802. The oligonucleotide of any one of the preceding Embodiments, wherein the second subdomain comprises no modified sugars comprising a 2′-OMe modification.
803. The oligonucleotide of any one of the preceding Embodiments, wherein the second subdomain comprises no modified sugars comprising a 2′-OR modification, wherein R is optionally substituted C₁₋₆ aliphatic.
804. The oligonucleotide of Embodiment 800, wherein each 2′-modified sugar is sugar comprising a 2′-F modification.
805. The oligonucleotide of any one of Embodiments 1-803, wherein the sugar of the opposite nucleoside is an acyclic sugar (e.g., a UNA sugar).
806. The oligonucleotide of any one of Embodiments 1-803, wherein the sugar of the opposite nucleoside comprises two 2′-H.
807. The oligonucleotide of any one of Embodiments 1-803, wherein the sugar of the opposite nucleoside comprises a 2′-OH.
808. The oligonucleotide of any one of Embodiments 1-803, wherein the sugar of the opposite nucleoside is a natural DNA sugar.
809. The oligonucleotide of any one of Embodiments 1-803, wherein the sugar of the opposite nucleoside comprises is modified.
810. The oligonucleotide of any one of Embodiments 1-803, wherein the sugar of the opposite nucleoside is sm04.
811. The oligonucleotide of any one of Embodiments 1-803, wherein the sugar of the opposite nucleoside is sm1l.
812. The oligonucleotide of any one of Embodiments 1-803, wherein the sugar of the opposite nucleoside is sm12.
813. The oligonucleotide of any one of Embodiments 1-803, wherein the sugar of the opposite nucleoside is sm13.
814. The oligonucleotide of any one of Embodiments 1-803, wherein the sugar of the opposite nucleoside is sm14.
815. The oligonucleotide of any one of Embodiments 1-803, wherein the sugar of the opposite nucleoside is sm15.
816. The oligonucleotide of any one of Embodiments 1-803, wherein the sugar of the opposite nucleoside is sm16.
817. The oligonucleotide of any one of Embodiments 1-803, wherein the sugar of the opposite nucleoside is sm17.
818. The oligonucleotide of any one of Embodiments 1-803, wherein the sugar of the opposite nucleoside is sm18.
819. The oligonucleotide of any one of Embodiments 1-803, wherein the sugar of the opposite nucleoside is sm19.
820. The oligonucleotide of any one of Embodiments 1-803, wherein the sugar of the opposite nucleoside comprises a 6-membered ring having 0-2 (0, 1, or 2) heteroatoms.
821. The oligonucleotide of any one of Embodiments 1-803, wherein the sugar of the opposite nucleoside is a bicyclic sugar.
822. The oligonucleotide of any one of Embodiments 1-803, wherein the sugar of the opposite nucleoside is a LNA sugar.
823. The oligonucleotide of any one of Embodiments 1-803, wherein the sugar of the opposite nucleoside is a cEt sugar.
824. The oligonucleotide of any one of Embodiments 1-803, wherein the sugar of the opposite nucleoside is a (S)-cEt sugar.
825. The oligonucleotide of any one of Embodiments 1-803, wherein the sugar of the opposite nucleoside has the structure of

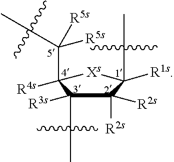

826. The oligonucleotide of Embodiment 825, wherein each R^{2s} is independently —H, —F, —OH or —OR^{ak}, wherein R^{ak} is optionally substituted C₁₋₆ aliphatic.
827. The oligonucleotide of any one of Embodiments 825-826, wherein the sugar has the structure of

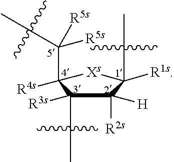

828. The oligonucleotide of any one of Embodiments 825-826, wherein the sugar has the structure of

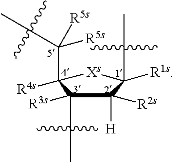

829. The oligonucleotide of any one of Embodiments 825-828, wherein R^{1s} is —H.
830. The oligonucleotide of any one of Embodiments 825-829, wherein R^{2s} is —H.
831. The oligonucleotide of any one of Embodiments 825-829, wherein R^{2s} is —F.
832. The oligonucleotide of any one of Embodiments 825-829, wherein R^{2s} is —OH.
833. The oligonucleotide of any one of Embodiments 825-829, wherein R^{2s} is —OR^{ak}, wherein R^{ak} is optionally substituted C₁₋₆ aliphatic.
834. The oligonucleotide of any one of Embodiments 825-829, wherein R^{2s} is —OMe.
835. The oligonucleotide of any one of Embodiments 825-829, wherein R^{2s} is —OCH₂CH₂OCH₃.
836. The oligonucleotide of any one of Embodiments 825-835, wherein R^{3s} is —H.
837. The oligonucleotide of any one of Embodiments 825-836, wherein R^{4s} is —H.
838. The oligonucleotide of any one of Embodiments 825-837, wherein one occurrence of R^{5s} is —H.
839. The oligonucleotide of any one of Embodiments 825-838, wherein the other occurrence of R^{5s} is —H.
840. The oligonucleotide of any one of Embodiments 825-838, wherein the other occurrence of R^{5s} is optionally substituted C₁₋₆ aliphatic.
841. The oligonucleotide of any one of Embodiments 1-803, wherein the sugar of the opposite nucleoside is optionally substituted

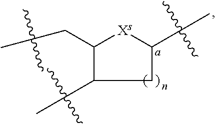

wherein position a is bonded to a nucleobase, wherein X^{s} is —O—, —S— or —Se—, and n is 0, 1, 2 or 3.
842. The oligonucleotide of any one of Embodiments 1-803, wherein the sugar of the opposite nucleoside is

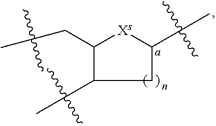

wherein position a is bonded to a nucleobase, wherein X^{s} is —O—, —S— or —Se—, and n is 0, 1,2or 3.
843. The oligonucleotide of any one of Embodiments 1-803, wherein the sugar of the opposite nucleoside is optionally substituted

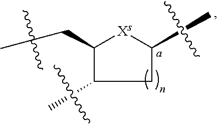

wherein position a is bonded to a nucleobase, wherein X^{s} is —O—, —S— or —Se—, and n is 0, 1, 2 or 3.
844. The oligonucleotide of any one of Embodiments 1-803, wherein the sugar of the opposite nucleoside is

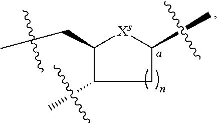

wherein position a is bonded to a nucleobase, wherein X^{s} is —O—, —S— or —Se—, and n is 0, 1,2or 3.
845. The oligonucleotide of any one of Embodiments 841-844, wherein n is 0.
846. The oligonucleotide of any one of Embodiments 841-844, wherein n is 1.
847. The oligonucleotide of any one of Embodiments 841-844, wherein n is 2.
848. The oligonucleotide of any one of Embodiments 841-844, wherein n is 3.
849. The oligonucleotide of any one of Embodiments 825-848, wherein X^{s} is —O—.
850. The oligonucleotide of any one of Embodiments 825-848, wherein X^{s} is —S—.
851. The oligonucleotide of any one of Embodiments 825-848, wherein X^{s} is optionally substituted —CH₂—.
852. The oligonucleotide of any one of Embodiments 825-848, wherein X^{s} is —CH₂—.
853. The oligonucleotide of any one of Embodiments 1-803, wherein the sugar of the opposite nucleoside comprises 2′-F.
854. The oligonucleotide of any one of Embodiments 1-803, wherein the sugar of the opposite nucleoside is a 2′-OR modified sugar wherein R is optionally substituted C₁₋₆ aliphatic.
855. The oligonucleotide of any one of Embodiments 1-803, wherein the sugar of the opposite nucleoside is a 2′-OMe modified sugar.
856. The oligonucleotide of any one of Embodiments 1-803, wherein the sugar of the opposite nucleoside is a 2′-MOE modified sugar.
857. The oligonucleotide of any one of Embodiments 1-856, wherein the second domain comprises N₁N₀N₋₁, wherein each of N₁, N₀ and N₋₁ is independently a nucleoside.
858. The oligonucleotide of any one of Embodiments 1-857, wherein the second domain comprises N₁N₀N₋₁, wherein each of N₁, N₀ and N₋₁ is independently a nucleoside, wherein when aligned with a target, N₀ is opposite to a target adenosine.
859. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises two 2′-H.
860. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises 2′-OH.
861. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) is a natural DNA sugar.
862. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises 2′-F.
863. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) comprises two 2′-H.
864. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) comprises 2′-OH.
865. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) is a natural DNA sugar.
866. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) comprises 2′-F.
867. The oligonucleotide of any one of Embodiments 1-803, wherein each of the sugar of the opposite nucleoside, the sugar of a nucleoside 5′-next to the opposite nucleoside (sugar of N₁ in 5′- . . . N₁N₀ . . . 3′, wherein when aligned with a target, N₀ is opposite to a target adenosine), and the sugar of a nucleoside 3′-next to the opposite nucleoside (sugar of N₋₁ in 5′- . . . N₀N₋₁ . . . 3′, wherein when aligned with a target, N₀ is opposite to a target adenosine) is independently a natural DNA sugar.
868. The oligonucleotide of any one of Embodiments 1-803, wherein the sugar of the opposite nucleoside is a natural DNA sugar, the sugar of a nucleoside 5′-next to the opposite nucleoside (sugar of N₁ in 5′- . . . N₁N₀ . . . 3′, wherein when aligned with a target, N₀ is opposite to a target adenosine) is a 2′-F modified sugar, and the sugar of a nucleoside 3′-next to the opposite nucleoside (sugar of N₋₁ in 5′- . . . N₀N₋₁ . . . 3′, wherein when aligned with a target, N₀ is opposite to a target adenosine) is a natural DNA sugar.
869. The oligonucleotide of any one of the preceding Embodiments, wherein the second subdomain comprise a 5′-end portion connected to 5′-side the opposite nucleoside.
870. The oligonucleotide of Embodiment 866, wherein the 5′-end portion comprises one or more mismatches or wobbles when aligned with a target nucleic acid for complementarity.
871. The oligonucleotide of Embodiment 866 or 870, wherein the 5′-end portion has a length of 1, 2 or 3 nucleobases.
872. The oligonucleotide of and one of Embodiments 866-871, wherein sugars of the 5′-end portion are selected from sugars having two 2′-H (e.g., natural DNA sugar) and 2′-F modified sugars.
873. The oligonucleotide of any one of the preceding Embodiments, wherein the second subdomain comprise a 3′-end portion connected to the 3′-side of the opposite nucleoside.
874. The oligonucleotide of Embodiment 873, wherein the 3′-end portion comprises one or more mismatches or wobbles when aligned with a target nucleic acid for complementarity.
875. The oligonucleotide of Embodiment 873, wherein the 3′-end portion comprises one or more mismatches and/or wobbles when aligned with a target nucleic acid for complementarity.
876. The oligonucleotide of Embodiment 873, wherein the 3′-end portion comprises one or more wobbles when aligned with a target nucleic acid for complementarity.
877. The oligonucleotide of Embodiment 873, wherein the 3′-end portion comprises an I or a derivative thereof.
878. The oligonucleotide of Embodiment 873, wherein the 3′-end portion comprises an I and an I-C wobble when aligned with a target nucleic acid for complementarity.
879. The oligonucleotide of any one of Embodiments 873-878, wherein the 3′-end portion has a length of 1, 2 or 3 nucleobases.
880. The oligonucleotide of and one of Embodiments 873-879, wherein sugars of the 3′-end portion are selected from sugars having two 2′-H (e.g., natural DNA sugar) and 2′-F modified sugars.
881. The oligonucleotide of and one of Embodiments 873-879, wherein sugars of the 3′-end portion are sugars having two 2′-H (e.g., natural DNA sugar).
882. The oligonucleotide of any one of the preceding Embodiments, wherein the second subdomain comprise about 1-10 (e.g., about 1, 2, 3, 4, 5, 6, 7, 8, 9, 10, 11, 12, 13, 14, 15, 16, 17, 18, 19, or 20, etc.) modified internucleotidic linkages.
883. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the second subdomain are modified internucleotidic linkages.
884. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the second subdomain are modified internucleotidic linkages.
885. The oligonucleotide of any one of the preceding Embodiments, wherein each modified internucleotidic linkages in the second subdomain is independently a chiral internucleotidic linkage.
886. The oligonucleotide of any one of the preceding Embodiments, wherein each modified internucleotidic linkages in the second subdomain is independently a phosphorothioate internucleotidic linkage or a non-negatively charged internucleotidic linkage.
887. The oligonucleotide of any one of the preceding Embodiments, wherein each modified internucleotidic linkages in the second subdomain is independently a phosphorothioate internucleotidic linkage or a neutral internucleotidic linkage.
888. The oligonucleotide of any one of the preceding Embodiments, wherein at least about 1-50 (e.g., about 5, 6, 7, 8, 9, or 10-about 10, 11, 12, 13, 14, 15, 16. 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is chirally controlled.
889. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 5% 90%, 95%, or 100%, etc.) of chiral internucleotidic linkages in the second subdomain is chirally controlled.
890. The oligonucleotide of any one of the preceding Embodiments, wherein each chiral internucleotidic linkage in the second subdomain is independently a chirally controlled internucleotidic linkage.
891. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is Sp.
892. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is Rp.
893. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second subdomain is Sp.
894. The oligonucleotide of any one of the preceding Embodiments, wherein each chiral internucleotidic linkages in the second subdomain is Sp.
895. The oligonucleotide of any one of the preceding Embodiments, wherein each internucleotidic linkage in the second subdomain is independently a modified internucleotidic linkage.
896. The oligonucleotide of any one of Embodiments 1-894, wherein the second subdomain comprises one or more natural phosphate linkages.
897. The oligonucleotide of any one of Embodiments 1-894, wherein the opposite nucleoside is connected to its 5′ immediate nucleoside through a natural phosphate linkage.
898. The oligonucleotide of any one of Embodiments 1-896, wherein the opposite nucleoside is connected to its 5′ immediate nucleoside through a modified internucleotidic linkage.
899. The oligonucleotide of any one of Embodiments 1-898, wherein the opposite nucleoside is connected to its 3′ immediate nucleoside through a modified internucleotidic linkage.
900. The oligonucleotide of any one of Embodiments 1-899, wherein the nucleoside (position -1) that is 3′ immediate to an opposite nucleoside (position 0) is connected to its 3′ immediate nucleoside (position -2) through a modified internucleotidic linkage.
901. The oligonucleotide of any one of Embodiments 898-900, wherein the modified internucleotidic linkage is a chiral internucleotidic linkage.
902. The oligonucleotide of any one of Embodiments 898-901, wherein the modified internucleotidic linkage is a phosphorothioate internucleotidic linkage.
903. The oligonucleotide of any one of Embodiments 898-901, wherein the modified internucleotidic linkage is a non-negatively charged internucleotidic linkage.
904. The oligonucleotide of any one of Embodiments 898-901, wherein the modified internucleotidic linkage is a neutral charged internucleotidic linkage.
905. The oligonucleotide of any one of Embodiments 901-904, wherein the chiral internucleotidic linkage is chirally controlled.
906. The oligonucleotide of any one of Embodiments 901-905, wherein the chiral internucleotidic linkage is Rp.
907. The oligonucleotide of any one of Embodiments 901-905, wherein the chiral internucleotidic linkage is Sp.
908. The oligonucleotide of any one of Embodiments 897-907, wherein the 5′ immediate nucleoside comprises a modified sugar.
909. The oligonucleotide of any one of Embodiments 897-907, wherein the 5′ immediate nucleoside comprises a modified sugar comprising a 2′-F modification.
910. The oligonucleotide of any one of Embodiments 897-907, wherein the 5′ immediate nucleoside comprises a sugar comprising two 2′-H (e.g., a natural DNA sugar).
911. The oligonucleotide of any one of Embodiments 1-894 and 896-910, wherein the opposite nucleoside is connected to its 3′ immediate nucleoside through a natural phosphate linkage.
912. The oligonucleotide of any one of Embodiments 1-894 and 896-910, wherein the opposite nucleoside is connected to its 3′ immediate nucleoside through a modified internucleotidic linkage.
913. The oligonucleotide of Embodiment 912, wherein the modified internucleotidic linkage is a chiral internucleotidic linkage.
914. The oligonucleotide of Embodiment 912 or 913, wherein the modified internucleotidic linkage is a phosphorothioate internucleotidic linkage.
915. The oligonucleotide of Embodiment 912 or 913, wherein the modified internucleotidic linkage is a non-negatively charged internucleotidic linkage.
916. The oligonucleotide of Embodiment 912 or 913, wherein the modified internucleotidic linkage is a neutral charged internucleotidic linkage.
917. The oligonucleotide of any one of Embodiments 913-916, wherein the chiral internucleotidic linkage is chirally controlled.
918. The oligonucleotide of any one of Embodiments 913-917, wherein the chiral internucleotidic linkage is Rp.
919. The oligonucleotide of any one of Embodiments 913-917, wherein the chiral internucleotidic linkage is Sp.
920. The oligonucleotide of any one of the preceding Embodiments, wherein the 3′ immediate nucleoside comprises a modified sugar.
921. The oligonucleotide of Embodiment 919, wherein the 3′ immediate nucleoside comprises a modified sugar comprising a 2′-F modification.
922. The oligonucleotide of Embodiment 919, wherein the 3′ immediate nucleoside comprises a sugar comprising two 2′-H (e.g., a natural DNA sugar).
923. The oligonucleotide of any one of the preceding Embodiments, wherein the 3′-immediate nucleoside comprises a base that is not G.
924. The oligonucleotide of any one of the preceding Embodiments, wherein the 3′-immediate nucleoside comprises a base that are less steric than G.
925. The oligonucleotide of any one of the preceding Embodiments, wherein the 3′-immediate nucleoside comprises a nucleobase which is or comprise Ring BA having the structure of formula BA-VI.
926. The oligonucleotide of any one of Embodiment 923-925, wherein Ring BA is the Ring BA of any one of Embodiments 477-633.
927. The oligonucleotide of any one of Embodiment 923-926, wherein the nucleobase is

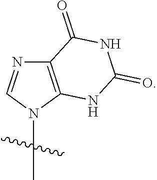 [SMILES: *n1cnc2c(=O)[nH]c(=O)[nH]c21]

928. The oligonucleotide of any one of Embodiment 923-926, wherein the nucleobase is

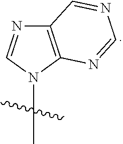 [SMILES: *n1cnc2cncnc21]

929. The oligonucleotide of any one of Embodiment 923-926, wherein the nucleobase is hypoxanthine.
930. The oligonucleotide of any one of the preceding Embodiments, wherein a target nucleic acid comprises 5′-CA-3′, wherein A is a target adenosine.
931. The oligonucleotide of any one of the preceding Embodiments, wherein the sugar in a 5′ immediate nucleoside is or comprises

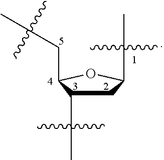

932. The oligonucleotide of any one of Embodiments 1-930, wherein the sugar in a 5′ immediate nucleoside is or comprises

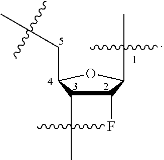

933. The oligonucleotide of any one of Embodiments 1-930, wherein the sugar in a 5′ immediate nucleoside is or comprises

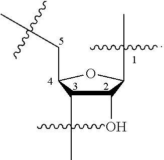

934. The oligonucleotide of any one of the preceding Embodiments, wherein the sugar in a nucleoside opposition to a target nucleoside is or comprises

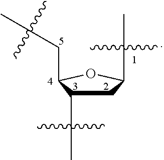

935. The oligonucleotide of any one of Embodiments 1-933, wherein the sugar in a nucleoside opposition to a target nucleoside is or comprises

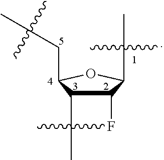

936. The oligonucleotide of any one of Embodiments 1-933, wherein the sugar in a nucleoside opposition to a target nucleoside is or comprises

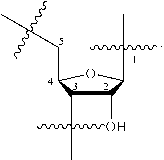

937. The oligonucleotide of any one of the preceding Embodiments, wherein the sugar in a 3′ immediate nucleoside is or comprises

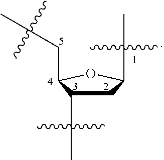

938. The oligonucleotide of any one of Embodiments 1-936, wherein the sugar in a 3′ immediate nucleoside is or comprises

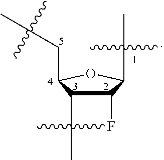

939. The oligonucleotide of any one of Embodiments 1-936, wherein the sugar in a 3′- immediate nucleoside is or comprises

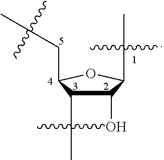

940. The oligonucleotide of any one of the preceding Embodiments, wherein the second subdomain can recruit, or promotes or contributes to recruitment of, an ADAR protein to a target nucleic acid.
941. The oligonucleotide of any one of the preceding Embodiments, wherein the second subdomain can interact, or promotes or contributes to interaction of, an ADAR protein with a target nucleic acid.
942. The oligonucleotide of any one of the preceding Embodiments, wherein the second subdomain contacts with a domain that have an enzymatic activity.
943. The oligonucleotide of any one of the preceding Embodiments, wherein the second subdomain contact with a domain that has a deaminase activity of ADAR1.
944. The oligonucleotide of any one of the preceding Embodiments, wherein the second subdomain contact with a domain that has a deaminase activity of ADAR2.
945. The oligonucleotide of any one of the preceding Embodiments, wherein the third subdomain has a length of about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
946. The oligonucleotide of any one of the preceding Embodiments, wherein the third subdomain has a length of about 1-10 (e.g., 1, 2, 3, 4, 5, 6, 7, 8, 9, or 10) nucleobases.
947. The oligonucleotide of any one of the preceding Embodiments, wherein the third subdomain comprises one or more (e.g., 1-10, 1, 2, 3, 4, 5, 6, 7, 8, 9, or 10, etc.) mismatches when the oligonucleotide is aligned with a target nucleic acid for complementarity.
948. The oligonucleotide of any one of the preceding Embodiments, wherein the third subdomain comprises two or more mismatches when the oligonucleotide is aligned with a target nucleic acid for complementarity.
949. The oligonucleotide of any one of Embodiments 1-947, wherein the third subdomain comprises one and no more than one mismatch when the oligonucleotide is aligned with a target nucleic acid for complementarity.
950. The oligonucleotide of any one of Embodiments 1-947, wherein the third subdomain comprises two and no more than two mismatches when the oligonucleotide is aligned with a target nucleic acid for complementarity.
951. The oligonucleotide of any one of the preceding Embodiments, wherein the third subdomain comprises one or more (e.g., 1-10, 1, 2, 3, 4, 5, 6, 7, 8. 9, or 10, etc.) bulges when the oligonucleotide is aligned with a target nucleic acid for complementarity.
952. The oligonucleotide of Embodiment 951, wherein each bulge independently comprises one or more base pairs that are not Watson-Crick or wobble pairs.
953. The oligonucleotide of any one of the preceding Embodiments, wherein the third subdomain comprises one or more (e.g., 1-10, 1, 2, 3, 4, 5, 6, 7, 8. 9, or 10, etc.) wobble pairs when the oligonucleotide is aligned with a target nucleic acid for complementarity.
954. The oligonucleotide of any one of the preceding Embodiments, wherein the third subdomain comprises two or more wobble pairs when the oligonucleotide is aligned with a target nucleic acid for complementarity.
955. The oligonucleotide of any one of the preceding Embodiments, wherein the third subdomain comprises two and no more than two wobble pairs when the oligonucleotide is aligned with a target nucleic acid for complementarity.
956. The oligonucleotide of any one of Embodiments 1-946, wherein the third subdomain is fully complementary to a target nucleic acid.
957. The oligonucleotide of any one of the preceding Embodiments, wherein the third subdomain comprises about 1-50 (e.g., about 5, 6, 7, 8, 9, or 10-about 10, 11, 12, 13, 14, 15, 16, 17, 18, 19, 20, 21, 22, 23, 24, 25. 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
958. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40% 50%, 60%, 65%, 70%, 75% 80%, 85%, 90%, 95%, or 100%, etc.) of sugars in the third subdomain are independently modified sugars with a modification that is not 2′-F.
959. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the third subdomain are independently modified sugars with a modification that is not 2′-F.
960. The oligonucleotide of any one of the preceding Embodiments, wherein the third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
961. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
962. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
963. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
964. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
965. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
966. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
967. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
968. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
969. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
970. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
971. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
972. The oligonucleotide of any one of Embodiments 1-962, wherein each sugar in the third subdomain independently comprises a 2′-OR modification, wherein R is optionally substituted C₁₋₆ aliphatic, or a 2′-O-L^{B}-4′ modification.
973. The oligonucleotide of Embodiment 972, wherein each sugar in the third subdomain independently comprises a 2′-OR modification, wherein R is optionally substituted C₁₋₆ aliphatic, or a 2′-O-L^{B}-4′ modification, wherein L^{B} is optionally substituted —CH₂-.
974. The oligonucleotide of Embodiment 972, wherein each sugar in the third subdomain independently comprises 2′-OMe.
975. The oligonucleotide of any one of the preceding Embodiments, wherein the third subdomain comprises a 5′-end portion having a length of about 1-8 nucleobases.
976. The oligonucleotide of Embodiment 975, wherein the 5′-end portion has a length of about 1, 2, or 3 nucleobases
977. The oligonucleotide of Embodiment 975 or 976, wherein the 5′-end portion is bonded to the second subdomain.
978. The oligonucleotide of any one of Embodiments 975-977, wherein one or more of the sugars in the 5′-end portion are independently modified sugars.
979. The oligonucleotide of Embodiment 978, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
980. The oligonucleotide of Embodiment 978, wherein one or more of the modified sugars independently comprises 2′-F.
981. The oligonucleotide of any one of Embodiments 975-977, wherein one or more sugars of the 5′-end portion independently comprise two 2′-H (e.g., natural DNA sugar).
982. The oligonucleotide of any one of Embodiments 975-981, wherein one or more sugars of the 5′-end portion independently comprise 2′-OH (e.g., natural RNA sugar).
983. The oligonucleotide of any one of Embodiments 975-977, wherein the sugars of the 5′-end portion independently comprise two 2′-H (e.g., natural DNA sugar) or a 2′-OH (e.g., natural RNA sugar).
984. The oligonucleotide of any one of Embodiments 975-977, wherein the sugars of the 5′-end portion are independently natural DNA or RNA sugars.
985. The oligonucleotide of any one of Embodiments 975-984, wherein the 5′-end portion comprises one or more mismatches.
986. The oligonucleotide of any one of Embodiments 975-985, wherein the 5′-end portion comprises one or more wobbles.
987. The oligonucleotide of any one of Embodiments 975-986, wherein the 5′-end portion is about 60-100% (e.g., 66%, 70%, 75%, 80%, 85%, 90%, 95%, or more) complementary to a target nucleic acid.
988. The oligonucleotide of any one of the preceding Embodiments, wherein the third subdomain comprises a 3′-end portion having a length of about 1-8 nucleobases.
989. The oligonucleotide of Embodiment 988, wherein the 3′-end portion has a length of about 1, 2, 3, or 4 nucleobases.
990. The oligonucleotide of Embodiment 988 or 989, wherein the 3′-end portion comprises the 3′-end nucleobase of the third subdomain.
991. The oligonucleotide of any one of Embodiments 988-990, wherein one or more of the sugars in the 3′-end portion are independently modified sugars.
992. The oligonucleotide of Embodiment 991, wherein the modified sugars are independently selected from a bicyclic sugar (e.g., a LNA sugar), an acyclic sugar (e.g.. a UNA sugar), a sugar with a 2′-OR modification, or a sugar with a 2′-N(R)₂ modification, wherein each R is independently optionally substituted C₁₋₆ aliphatic.
993. The oligonucleotide of any one of Embodiments 991-992, wherein one or more modified sugars independently comprises 2′-F.
994. The oligonucleotide of any one of Embodiments 991-992, wherein at least 20%, 25%, 30%, 35%, 40%, 50%, 60%, 70%, 75%, 80%, 90%, or 95% sugars in the third subdomain independently comprise 2′-F.
995. The oligonucleotide of any one of Embodiments 991-994, wherein one or more sugars in the 3′-end portion independently comprise a 2′-OR modification, wherein R is optionally substituted C₁₋₆ aliphatic, or a 2′-O-L^{B}-4′ modification.
996. The oligonucleotide of Embodiment 995, wherein each sugar in the 3′-end portion independently comprises a 2′-OR modification, wherein R is optionally substituted C₁₋₆ aliphatic, or a 2′-O-L^{B}-4′ modification.
997. The oligonucleotide of any one of Embodiments 995-996, wherein L^{B} is optionally substituted —CH₂-.
998. The oligonucleotide of any one of Embodiments 995-996, wherein L^{B} is —CH₂-.
999. The oligonucleotide of Embodiment 995, wherein each sugar in the 3′-end portion independently comprises 2′-OMe.
1000. The oligonucleotide of any one of Embodiments 988-999, wherein the 3′-end portion comprises one or more mismatches.
1001. The oligonucleotide of any one of Embodiments 988-1000, wherein the 3′-end portion comprises one or more wobbles.
1002. The oligonucleotide of any one of Embodiments 988-1001, wherein the 3′-end portion is about 60-100% (e.g., 66%, 70%, 75%, 80%, 85%, 90%, 95%, or more) complementary to a target nucleic acid.
1003. The oligonucleotide of any one of the preceding Embodiments, wherein the third subdomain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
1004. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the third subdomain are modified internucleotidic linkages.
1005. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the third subdomain are modified internucleotidic linkages.
1006. The oligonucleotide of any one of the preceding Embodiments, wherein each modified internucleotidic linkages is independently a chiral internucleotidic linkage.
1007. The oligonucleotide of any one of the preceding Embodiments, wherein the internucleotidic linkage between the last and the second last nucleosides of the third subdomain is a non-negatively charged internucleotidic linkage.
1008. The oligonucleotide of any one of the preceding Embodiments, wherein each modified internucleotidic linkages is independently a phosphorothioate internucleotidic linkage or a non-negatively charged internucleotidic linkage.
1009. The oligonucleotide of any one of the preceding Embodiments, wherein each modified internucleotidic linkages is independently a phosphorothioate internucleotidic linkage or a neutral internucleotidic linkage.
1010. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the third subdomain is chirally controlled.
1011. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the third subdomain is chirally controlled.
1012. The oligonucleotide of any one of the preceding Embodiments, wherein the internucleotidic linkage between the last and the second last nucleosides of the third subdomain is chirally controlled.
1013. The oligonucleotide of any one of the preceding Embodiments, wherein each chiral internucleotidic linkage is independently a chirally controlled internucleotidic linkage.
1014. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the third subdomain is Sp.
1015. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 5% 90%, 95%, or 100%, etc.) of chiral internucleotidic linkages in the third subdomain is Sp.
1016. The oligonucleotide of any one of the preceding Embodiments, wherein each chiral internucleotidic linkages in the third subdomain is Sp.
1017. The oligonucleotide of any one of Embodiments 1-1015, wherein the internucleotidic linkage between the last and the second lase nucleosides of the third subdomain is Rp.
1018. The oligonucleotide of any one of the preceding Embodiments, wherein the internucleotidic linkage linking the last nucleoside of the second subdomain and the first nucleoside of the third subdomain is a non-negatively charged internucleotidic linkage.
1019. The oligonucleotide of any one of the preceding Embodiments, wherein the internucleotidic linkage at position -2 is a non-negatively charged internucleotidic linkage.
1020. The oligonucleotide of any one of Embodiments 1018-1019, wherein the non-negatively charged internucleotidic linkage is chirally controlled.
1021. The oligonucleotide of Embodiment 1020, wherein the non-negatively charged internucleotidic linkage is Rp.
1022. The oligonucleotide of Embodiment 1020, wherein the non-negatively charged internucleotidic linkage is Sp.
1023. The oligonucleotide of any one of the preceding Embodiments, wherein each internucleotidic linkage in the third subdomain is independently a modified internucleotidic linkage.
1024. The oligonucleotide of any one of Embodiments 1-1022, wherein the third subdomain comprises one or more natural phosphate linkages.
1025. The oligonucleotide of any one of the preceding Embodiments, wherein the third subdomain can recruit, or promotes or contributes to recruitment of, an ADAR protein to a target nucleic acid.
1026. The oligonucleotide of any one of the preceding Embodiments, wherein the third subdomain can interact, or promotes or contributes to interaction of, an ADAR protein with a target nucleic acid.
1027. The oligonucleotide of any one of the preceding Embodiments, wherein the third subdomain contacts with a domain that have an enzymatic activity.
1028. The oligonucleotide of any one of the preceding Embodiments, wherein the third subdomain contact with a domain that has a deaminase activity of ADAR1.
1029. The oligonucleotide of any one of the preceding Embodiments, wherein the third subdomain contact with a domain that has a deaminase activity of ADAR2.
1030. The oligonucleotide of any one of the preceding Embodiments, wherein each wobble base pair is independently G-U, I-A, G-A, I-U, I-C, I-T, A-A, or reverse A-T.
1031. The oligonucleotide of any one of the preceding Embodiments, wherein each wobble base pair is independently G-U, I-A. G-A, I-U, or I-C.
1032. The oligonucleotide of any one of the preceding Embodiments, wherein each cyclic sugar or each sugar is independently optionally substituted

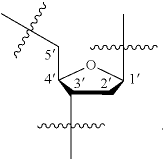

1033. The oligonucleotide of any one of the preceding Embodiments, wherein each cyclic sugar or each sugar independently has the structure of

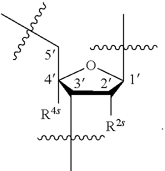

1034. The oligonucleotide of Embodiment 1033, wherein the oligonucleotide comprises one or more sugars wherein R^{2s} and R^{4s} are H.
1035. The oligonucleotide of any one of Embodiments 1033-1034, wherein the oligonucleotide comprises one or more sugars wherein R^{2s} is —OR, and R^{4s} is H.
1036. The oligonucleotide of any one of Embodiments 1033-1035, wherein the oligonucleotide comprises one or more sugars wherein R^{2s} is —OR, wherein R is optionally substituted C₁₋₄ alkyl and R^{4s} is H.
1037. The oligonucleotide of any one of Embodiments 1033-1036, wherein the oligonucleotide comprises one or more sugars wherein R^{2s} is —OMe and R^{4s} is H.
1038. The oligonucleotide of any one of Embodiments 1033-1037, wherein the oligonucleotide comprises one or more sugars wherein R^{2s} is —F and R^{4s} is H.
1039. The oligonucleotide of any one of Embodiments 1033-1038, wherein the oligonucleotide comprises one or more sugars wherein R^{4s} and R^{2s} are forming a bridge having the structure of optionally substituted 2′-O—CH₂-4′.
1040. The oligonucleotide of any one of Embodiments 1033-1038, wherein the oligonucleotide comprises one or more sugars wherein R^{4s} and R^{2s} are forming a bridge having the structure of 2′-O—CH₂-4′.
1041. The oligonucleotide of any one of the preceding Embodiments, wherein the oligonucleotide comprises an additional chemical moiety.
1042. The oligonucleotide of any one of the preceding Embodiments, wherein the oligonucleotide comprises a targeting moiety.
1043. The oligonucleotide of any one of the preceding Embodiments, wherein the oligonucleotide comprises a carbohydrate moiety.
1044. The oligonucleotide of any one of Embodiments 1039-1043, wherein the moiety is or comprises a ligand for an asialoglycoprotein receptor.
1045. The oligonucleotide of any one of Embodiments 1039-1044, wherein the moiety is or comprises GalNAc or a derivative thereof.
1046. The oligonucleotide of any one of Embodiments 1039-1045, wherein the moiety is or comprises optionally substituted

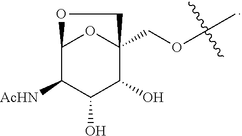 [SMILES: *OC[C@@]12CO[C@@H](O1)[C@H](NC(C)=O)[C@@H](O)[C@H]2O]

1047. The oligonucleotide of any one of Embodiments 1039-1045, wherein the moiety is or comprises optionally substituted

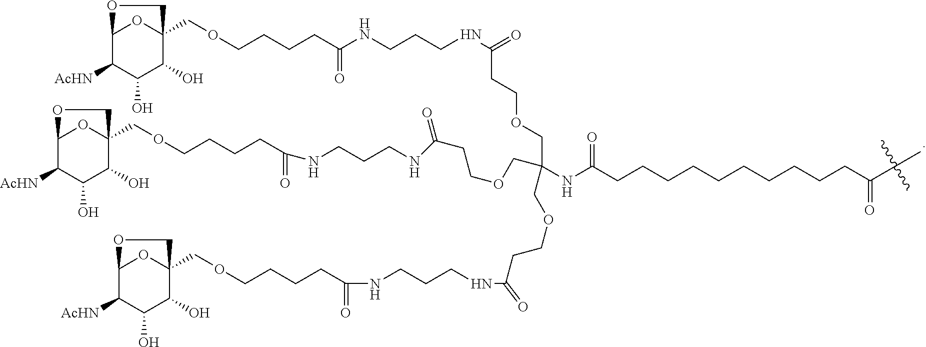 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O]

1048. The oligonucleotide of any one of Embodiments 1039-1047, wherein the moiety is connected to an oligonucleotide chain through a linker.
1049. The oligonucleotide of Embodiment 1048, wherein the linker is or comprises L001.
1050. The oligonucleotide of Embodiment 1049, wherein L001 is connected to 5′-end 5′-carbon of an oligonucleotide chain through a phosphate group
1051. The oligonucleotide of any one of the preceding Embodiments, wherein an additional chemical moiety is or comprises a nucleic acid moiety.
1052. The oligonucleotide of Embodiment 1051, wherein the nucleic acid is or comprises an aptamer.
1053. The oligonucleotide of any one of the preceding Embodiments, wherein the oligonucleotide is in a salt form.
1054. The oligonucleotide of any one of the preceding Embodiments, wherein the oligonucleotide is in a pharmaceutically acceptable salt form.
1055. The oligonucleotide of any one of the preceding Embodiments, wherein the oligonucleotide is in a sodium salt form.
1056. The oligonucleotide of any one of the preceding Embodiments, wherein the oligonucleotide is in an ammonium salt form.
1057. The oligonucleotide of any one of the preceding Embodiments, wherein if any, at least one or each neutral internucleotidic linkage is independently n001.
1058. The oligonucleotide of any one of the preceding Embodiments, wherein if any, each non-negatively charged internucleotidic linkage is independently n001.
1059. The oligonucleotide of any one of the preceding Embodiments, wherein no more than 5, 6, 7, 8, 9, 10, 11 or 12 nucleosides 3′ to a nucleoside opposite to a target adenosine.
1060. The oligonucleotide of any one of the preceding Embodiments, wherein no more than 5, 6, 7, 8, 9, 10, 11 or 12 nucleosides 3′ to a nucleoside opposite to a target nucleoside, wherein each of the nucleosides is independently optionally substituted A, T, C, G, U, or a tautomer thereof.
1061. The oligonucleotide of any one of the preceding Embodiments, wherein about 50%-100% (e.g., about or at least about 50%, 55%, 60%, 65%, 70%, 75%, 80%, 85%, 90%, 95%) of internucleotidic linkages 3′ to a nucleoside opposite to a target adenosine are each independently a modified internucleotidic linkage.
1062. The oligonucleotide of any one of the preceding Embodiments, wherein about 50%-100% (e.g., about or at least about 50%, 55%, 60%, 65%, 70%, 75%, 80%, 85%, 90%, 95%) of internucleotidic linkages 3′ to a nucleoside opposite to a target adenosine are each independently a phosphorothioate internucleotidic linkage or a non-negatively charged internucleotidic linkage.
1063. The oligonucleotide of any one of the preceding Embodiments, wherein no more than 1, 2, or 3 internucleotidic linkages 3′ to a nucleoside opposite to a target adenosine are natural phosphate linkages.
1064. The oligonucleotide of any one of the preceding Embodiments, wherein no more than 1, 2, or 3 internucleotidic linkages 3′ to a nucleoside opposite to a target adenosine are Rp internucleotidic linkages.
1065. The oligonucleotide of any one of the preceding Embodiments, wherein no more than 1, 2, or 3 internucleotidic linkages 3′ to a nucleoside opposite to a target adenosine are Rp phosphorothioate internucleotidic linkages.
1066. The oligonucleotide of any one of the preceding Embodiments, wherein the internucleotidic linkage between a nucleoside opposite to a target nucleoside and its 3′ immediate nucleoside (considered a -1 position) is a stereorandom phosphorothioate internucleotidic linkage.
1067. The oligonucleotide of any one of Embodiments 1-1065, wherein the internucleotidic linkage between a nucleoside opposite to a target nucleoside and its 3′ immediate nucleoside (considered a -1 position) is a chirally controlled Rp phosphorothioate internucleotidic linkage.
1068. The oligonucleotide of any one of Embodiments 1-1065, wherein the internucleotidic linkage between a nucleoside opposite to a target nucleoside and its 3′ immediate nucleoside (considered a -1 position) is a chirally controlled Sp phosphorothioate internucleotidic linkage
1069. The oligonucleotide of any one of Embodiments 1-1065, wherein an internucleotidic linkage bonded to a nucleoside opposite to a target nucleoside at the 3′-position of its sugar (considered a -1 position) is a Rp phosphorothioate internucleotidic linkage, and optionally the only Rp phosphorothioate internucleotidic linkage 3′ to a nucleoside opposite to a target adenosine.
1070. The oligonucleotide of any one of Embodiments 1-1065, wherein an internucleotidic linkage bonded to a nucleoside opposite to a target nucleoside at the 3′-position of its sugar (considered a −1 position) is a Sp phosphorothioate internucleotidic linkage.
1071. The oligonucleotide of any one of Embodiments 1-1065, wherein an internucleotidic linkage bonded to a nucleoside opposite to a target nucleoside at the 3′-position of its sugar (considered a −1 position) is a stereorandom phosphorothioate internucleotidic linkage.
1072. The oligonucleotide of any one of Embodiments 1-1065, wherein the internucleotidic linkage between a 3′ immediate nucleoside of a nucleoside opposite to a target nucleoside and the next 3′ immediate nucleoside (e.g., position −2 between N₋₁ and N₋₂ of 5′- . . . N₀N₋₁N₋₂ . . . −3′ wherein No represents a nucleoside opposite to a target nucleoside) is a non-negatively charged internucleotidic linkage.
1073. The oligonucleotide of Embodiment 1072, wherein the non-negatively charged internucleotidic linkage is stereorandom.
1074. The oligonucleotide of Embodiment 1072, wherein the non-negatively charged internucleotidic linkage is chirally controlled.
1075. The oligonucleotide of Embodiment 1072, wherein the non-negatively charged internucleotidic linkage is chirally controlled and is Sp.
1076. The oligonucleotide of Embodiment 1072, wherein the non-negatively charged internucleotidic linkage is chirally controlled and is Rp.
1077. The oligonucleotide of any one of Embodiments 1072-1076, wherein a non-negatively charged internucleotidic linkage is phosphoryl guanidine internucleotidic linkage.
1078. The oligonucleotide of any one of Embodiments 1072-1076, wherein a non-negatively charged internucleotidic linkage is n001.
1079. The oligonucleotide of any one of Embodiments 1072-1076, wherein a non-negatively charged internucleotidic linkage is n004, n008, n025 or n026.
1080. The oligonucleotide of any one of the preceding Embodiments, wherein the first internucleotidic linkage is a non-negatively charged internucleotidic linkage.
1081. The oligonucleotide of Embodiment 1080, wherein the non-negatively charged internucleotidic linkage is stereorandom.
1082. The oligonucleotide of Embodiment 1080, wherein the non-negatively charged internucleotidic linkage is chirally controlled.
1083. The oligonucleotide of Embodiment 1080, wherein the non-negatively charged internucleotidic linkage is chirally controlled and is Sp.
1084. The oligonucleotide of Embodiment 1080, wherein the non-negatively charged internucleotidic linkage is chirally controlled and is Rp.
1085. The oligonucleotide of any one of Embodiments 1080-1084, wherein a non-negatively charged internucleotidic linkage is phosphoryl guanidine internucleotidic linkage.
1086. The oligonucleotide of any one of Embodiments 1080-1084, wherein a non-negatively charged internucleotidic linkage is n001.
1087. The oligonucleotide of any one of Embodiments 1080-1084, wherein a non-negatively charged internucleotidic linkage is n004, n008, n025, n026.
1088. The oligonucleotide of any one of the preceding Embodiments, wherein the last internucleotidic linkage is a non-negatively charged internucleotidic linkage.
1089. The oligonucleotide of Embodiment 1088, wherein the non-negatively charged internucleotidic linkage is stereorandom.
1090. The oligonucleotide of Embodiment 1088, wherein the non-negatively charged internucleotidic linkage is chirally controlled.
1091. The oligonucleotide of Embodiment 1088, wherein the non-negatively charged internucleotidic linkage is chirally controlled and is Sp.
1092. The oligonucleotide of Embodiment 1088, wherein the non-negatively charged internucleotidic linkage is chirally controlled and is Rp.
1093. The oligonucleotide of any one of Embodiments 1088-1092, wherein a non-negatively charged internucleotidic linkage is a phosphoryl guanidine internucleotidic linkage.
1094. The oligonucleotide of any one of Embodiments 1088-1092, wherein a non-negatively charged internucleotidic linkage is n004, n008, n025, n026.
1095. The oligonucleotide of any one of Embodiments 1088-1092, wherein a non-negatively charged internucleotidic linkage is n001.
1096. The oligonucleotide of any one of the preceding Embodiments, wherein an internucleotidic linkage at position −3 relative to a nucleoside opposite to a target adenosine is not a Rp phosphorothioate internucleotidic linkage.
1097. The oligonucleotide of any one of the preceding Embodiments, wherein an internucleotidic linkage at position −6 relative to a nucleoside opposite to a target adenosine is not a Rp phosphorothioate internucleotidic linkage.
1098. The oligonucleotide of any one of the preceding Embodiments, wherein an internucleotidic linkage at position −4 and/or −5 relative to a nucleoside opposite to a target nucleoside is a modified internucleotidic linkage, e.g., a phosphorothioate internucleotidic linkage.
1099. The oligonucleotide of any one of the preceding Embodiments, wherein a nucleoside opposite to a target nucleoside is at position 2, 3, 4, 5, 6, 7, 8, 9, 10, 11, 12, 13, 14, 15, 16, 17, 18, 19, 20, 21, 22, 23, 24, 25, 26, 27. 28, 29 or 30 or more from the 5′-end.
1100. The oligonucleotide of any one of the preceding Embodiments, wherein a nucleoside opposite to a target nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 3′-end.
1101. The oligonucleotide of Embodiment 1099 or 1100, wherein the position is position 4.
1102. The oligonucleotide of Embodiment 1099 or 1100, wherein the position is position 5.
1103. The oligonucleotide of Embodiment 1099 or 1100, wherein the position is position 6.
1104. The oligonucleotide of Embodiment 1099 or 1100, wherein the position is position 7.
1105. The oligonucleotide of Embodiment 1099 or 1100, wherein the position is position 8.
1106. The oligonucleotide of Embodiment 1099 or 1100, wherein the position is position 9.
1107. The oligonucleotide of Embodiment 1099 or 1100, wherein the position is position 10.
1108. The oligonucleotide of any one of the preceding Embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target adenosine are each independently a modified internucleotidic linkage, which is optionally chirally controlled.
1109. The oligonucleotide of any one of the preceding Embodiments, about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each chirally controlled and are Sp.
1110. The oligonucleotide of any one of the preceding Embodiments, wherein no or no more than 1, 2, or 3 internucleotidic linkages 5′ to a nucleoside opposite to a target adenosine are natural phosphate linkages
1111. The oligonucleotide of any one of the preceding Embodiments, an internucleotidic linkage at position +5 relative to a nucleoside opposite to a target nucleoside (e.g., for . . . N₊₅N₊₄N₊₃N₊₂N₊₁N₀ . . . the internucleotidic linkage linking N₊₄ and N₊₅ wherein N₀ is a nucleoside opposite to a target nucleoside) is not a Rp phosphorothioate internucleotidic linkage.
1112. The oligonucleotide of any one of the preceding Embodiments, wherein one or more or all internucleotidic linkages at positions +6 to +8 relative to a nucleoside opposite to a target adenosine are each independently a modified internucleotidic linkage, optionally chirally controlled.
1113. The oligonucleotide of any one of the preceding Embodiments, wherein one or more or all internucleotidic linkages at positions +6 to +8 relative to a nucleoside opposite to a target adenosine are each independently a phosphorothioate internucleotidic linkage, optionally chirally controlled.
1114. The oligonucleotide of any one of the preceding Embodiments, wherein one or more or all internucleotidic linkages at positions +6, +7, +8, +9, and +11 relative to a nucleoside opposite to a target adenosine are each independently Rp phosphorothioate internucleotidic linkages.
1115. The oligonucleotide of any one of the preceding Embodiments, wherein one or more or all internucleotidic linkages at positions +5, +6, +7, +8, and +9 relative to a nucleoside opposite to a target adenosine are each independently Sp phosphorothioate internucleotidic linkages.
1116. The oligonucleotide of any one of the preceding Embodiments, comprising an optionally protected nucleobase of a nucleoside selected from b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b003I, b004I, b0141, [3nT], [3ne5U], [3nfl5U], [3npry5U], [3ncn5U], [napth6o8A], [ipr6o8A], [c7In], [c39z481n], [z2c3In], [z5C], and zdnp, or a nucleobase selected from Table BA-1.
1117. The oligonucleotide of any one of the preceding Embodiments, comprising optionally protected nucleobase b001U.
1118. The oligonucleotide of any one of the preceding Embodiments, comprising optionally protected nucleobase b002U.
1119. The oligonucleotide of any one of the preceding Embodiments, comprising optionally protected nucleobase b003U.
1120. The oligonucleotide of any one of the preceding Embodiments, comprising optionally protected nucleobase b004U.
1121. The oligonucleotide of any one of the preceding Embodiments, comprising optionally protected nucleobase b005U.
1122. The oligonucleotide of any one of the preceding Embodiments, comprising optionally protected nucleobase b006U.
1123. The oligonucleotide of any one of the preceding Embodiments, comprising optionally protected nucleobase b007U.
1124. The oligonucleotide of any one of the preceding Embodiments, comprising optionally protected nucleobase b008U.
1125. The oligonucleotide of any one of the preceding Embodiments, comprising optionally protected nucleobase b009U.
1126. The oligonucleotide of any one of the preceding Embodiments, comprising optionally protected nucleobase b011U.
1127. The oligonucleotide of any one of the preceding Embodiments, comprising optionally protected nucleobase b012U.
1128. The oligonucleotide of any one of the preceding Embodiments, comprising optionally protected nucleobase b013U.
1129. The oligonucleotide of any one of the preceding Embodiments, comprising optionally protected nucleobase b014U.
1130. The oligonucleotide of any one of the preceding Embodiments, comprising optionally protected nucleobase b015U.
1131. The oligonucleotide of any one of the preceding Embodiments, comprising optionally protected nucleobase b001A.
1132. The oligonucleotide of any one of the preceding Embodiments, comprising optionally protected nucleobase b002A.
1133. The oligonucleotide of any one of the preceding Embodiments, comprising optionally protected nucleobase b003A.
1134. The oligonucleotide of any one of the preceding Embodiments, comprising optionally protected nucleobase b004A.
1135. The oligonucleotide of any one of the preceding Embodiments, comprising optionally protected nucleobase b005A.
1136. The oligonucleotide of any one of the preceding Embodiments, comprising optionally protected nucleobase b006A.
1137. The oligonucleotide of any one of the preceding Embodiments, comprising optionally protected nucleobase b007A.
1138. The oligonucleotide of any one of the preceding Embodiments, comprising optionally protected nucleobase b001G.
1139. The oligonucleotide of any one of the preceding Embodiments, comprising optionally protected nucleobase b002G.
1140. The oligonucleotide of any one of the preceding Embodiments, comprising optionally protected nucleobase b001C.
1141. The oligonucleotide of any one of the preceding Embodiments, comprising optionally protected nucleobase b002C.
1142. The oligonucleotide of any one of the preceding Embodiments, comprising optionally protected nucleobase b003C.
1143. The oligonucleotide of any one of the preceding Embodiments, comprising optionally protected nucleobase b004C.
1144. The oligonucleotide of any one of the preceding Embodiments, comprising optionally protected nucleobase b005C.
1145. The oligonucleotide of any one of the preceding Embodiments, comprising optionally protected nucleobase b006C.
1146. The oligonucleotide of any one of the preceding Embodiments, comprising optionally protected nucleobase b007C.
1147. The oligonucleotide of any one of the preceding Embodiments, comprising optionally protected nucleobase b008C.
1148. The oligonucleotide of any one of the preceding Embodiments, comprising optionally protected nucleobase b009C.
1149. The oligonucleotide of any one of the preceding Embodiments, comprising optionally protected nucleobase b002I.
1150. The oligonucleotide of any one of the preceding Embodiments, comprising optionally protected nucleobase b003I.
1151. The oligonucleotide of any one of the preceding Embodiments, comprising optionally protected nucleobase b004I.
1152. The oligonucleotide of any one of the preceding Embodiments, comprising optionally protected nucleobase b014I.
1153. The oligonucleotide of any one of the preceding Embodiments, comprising optionally protected nucleobase [3nT].
1154. The oligonucleotide of any one of the preceding Embodiments, comprising optionally protected nucleobase [3ne5U].
1155. The oligonucleotide of any one of the preceding Embodiments, comprising optionally protected nucleobase [3nfl5U].
1156. The oligonucleotide of any one of the preceding Embodiments, comprising optionally protected nucleobase [3npry5U].
1157. The oligonucleotide of any one of the preceding Embodiments, comprising optionally protected nucleobase [3ncn5U].
1158. The oligonucleotide of any one of the preceding Embodiments, comprising optionally protected nucleobase [napth6o8A].
1159. The oligonucleotide of any one of the preceding Embodiments, comprising optionally protected nucleobase [ipr6o8A].
1160. The oligonucleotide of any one of the preceding Embodiments, comprising optionally protected nucleobase [c7In].
1161. The oligonucleotide of any one of the preceding Embodiments, comprising optionally protected nucleobase [c39z48In].
1162. The oligonucleotide of any one of the preceding Embodiments, comprising optionally protected nucleobase [z2c3In].
1163. The oligonucleotide of any one of the preceding Embodiments, comprising optionally protected nucleobase [z5C].
1164. The oligonucleotide of any one of the preceding Embodiments, comprising optionally protected nucleobase and zdnp.

1165. The oligonucleotide of any one of the preceding Embodiments, comprising an optionally protected nucleoside selected from aC, b001U, b002U, b003U, b004U, b005U, b006U, b007U, b008U, b008Usm15, b008Usm19, b009U, b010U, b011U, b012U, b013U, b014U, b015U, b001A, b001Asm15, b001rA, b002A, b003A, b004A, b005A, b006A, b007A, b001G, b002G, b001C, b002C, b003C, b003mC, b004C, b005C, b006C, b007C, b008C, b002I, b003I, b004I, b0141, [3nT], [3ne5U], [3nfl5U], [3npry5U], [3ncn5U], [napth6o8A], [ipr6o8A], [c7ln], [c39z481n], [z2c3ln], [z5C], AsmO1, Gsm01, 5MSfC, Usm04, 5MRdT, Csm04, Csm 11, Gsm 11, Tsm 11, b009Csm11, b009Csm12, Gsm12, Tsm12, Csm12, rCsm13, rCsm 14, Csm15, Csm16, Csm17, Csm19, L034, zdnp, and Tsm18.

1166. The oligonucleotide of any one of the preceding Embodiments, comprising an optionally protected sugar of a nucleoside selected from Asm01, Gsm01, 5MSfC, Usm04, 5MRdT, Csm15, Csm16, rCsm14, Csm17 and Tsm18.
1167. The oligonucleotide of any one of the preceding Embodiments, comprising optionally protected nucleoside aC.
1168. The oligonucleotide of any one of the preceding Embodiments, comprising optionally protected nucleoside b001U.
1169. The oligonucleotide of any one of the preceding Embodiments, comprising optionally protected nucleoside b002U.
1170. The oligonucleotide of any one of the preceding Embodiments, comprising optionally protected nucleoside b003U.
1171. The oligonucleotide of any one of the preceding Embodiments, comprising optionally protected nucleoside b004U.
1172. The oligonucleotide of any one of the preceding Embodiments, comprising optionally protected nucleoside b005U.
1173. The oligonucleotide of any one of the preceding Embodiments, comprising optionally protected nucleoside b006U.
1174. The oligonucleotide of any one of the preceding Embodiments, comprising optionally protected nucleoside b007U.
1175. The oligonucleotide of any one of the preceding Embodiments, comprising optionally protected nucleoside b008U.
1176. The oligonucleotide of any one of the preceding Embodiments, comprising optionally protected nucleoside b009U.
1177. The oligonucleotide of any one of the preceding Embodiments, comprising optionally protected nucleoside b010U.
1178. The oligonucleotide of any one of the preceding Embodiments, comprising optionally protected nucleoside b011U.
1179. The oligonucleotide of any one of the preceding Embodiments, comprising optionally protected nucleoside b012U.
1180. The oligonucleotide of any one of the preceding Embodiments, comprising optionally protected nucleoside b013U.
1181. The oligonucleotide of any one of the preceding Embodiments, comprising optionally protected nucleoside b014U.
1182. The oligonucleotide of any one of the preceding Embodiments, comprising optionally protected nucleoside b015U.
1183. The oligonucleotide of any one of the preceding Embodiments, comprising optionally protected nucleoside b001A.
1184. The oligonucleotide of any one of the preceding Embodiments, comprising optionally protected nucleoside b001rA.
1185. The oligonucleotide of any one of the preceding Embodiments, comprising optionally protected nucleoside b002A.
1186. The oligonucleotide of any one of the preceding Embodiments, comprising optionally protected nucleoside b003A.
1187. The oligonucleotide of any one of the preceding Embodiments, comprising optionally protected nucleoside b004A.
1188. The oligonucleotide of any one of the preceding Embodiments, comprising optionally protected nucleoside b005A.
1189. The oligonucleotide of any one of the preceding Embodiments, comprising optionally protected nucleoside b006A.
1190. The oligonucleotide of any one of the preceding Embodiments, comprising optionally protected nucleoside b007A.
1191. The oligonucleotide of any one of the preceding Embodiments, comprising optionally protected nucleoside b001G.
1192. The oligonucleotide of any one of the preceding Embodiments, comprising optionally protected nucleoside b002G.
1193. The oligonucleotide of any one of the preceding Embodiments, comprising optionally protected nucleoside b001C.
1194. The oligonucleotide of any one of the preceding Embodiments, comprising optionally protected nucleoside b002C.
1195. The oligonucleotide of any one of the preceding Embodiments, comprising optionally protected nucleoside b003C.
1196. The oligonucleotide of any one of the preceding Embodiments, comprising optionally protected nucleoside b003mC.
1197. The oligonucleotide of any one of the preceding Embodiments, comprising optionally protected nucleoside b004C.
1198. The oligonucleotide of any one of the preceding Embodiments, comprising optionally protected nucleoside b005C.
1199. The oligonucleotide of any one of the preceding Embodiments, comprising optionally protected nucleoside b006C.
1200. The oligonucleotide of any one of the preceding Embodiments, comprising optionally protected nucleoside b007C.
1201. The oligonucleotide of any one of the preceding Embodiments, comprising optionally protected nucleoside b008C.
1202. The oligonucleotide of any one of the preceding Embodiments, comprising optionally protected nucleoside b002I.
1203. The oligonucleotide of any one of the preceding Embodiments, comprising optionally protected nucleoside b003I.
1204. The oligonucleotide of any one of the preceding Embodiments, comprising optionally protected nucleoside b004I.
1205. The oligonucleotide of any one of the preceding Embodiments, comprising optionally protected nucleoside b014I.
1206. The oligonucleotide of any one of the preceding Embodiments, comprising optionally protected nucleoside [3nT].
1207. The oligonucleotide of any one of the preceding Embodiments, comprising optionally protected nucleoside [3ne5U].
1208. The oligonucleotide of any one of the preceding Embodiments, comprising optionally protected nucleoside [3nfl5U].
1209. The oligonucleotide of any one of the preceding Embodiments, comprising optionally protected nucleoside [3npry5U].
1210. The oligonucleotide of any one of the preceding Embodiments, comprising optionally protected nucleoside [3ncn5U].
1211. The oligonucleotide of any one of the preceding Embodiments, comprising optionally protected nucleoside [napth6o8A].
1212. The oligonucleotide of any one of the preceding Embodiments, comprising optionally protected nucleoside [ipr6o8A].
1213. The oligonucleotide of any one of the preceding Embodiments, comprising optionally protected nucleoside [c7In].
1214. The oligonucleotide of any one of the preceding Embodiments, comprising optionally protected nucleoside [c39z48In].
1215. The oligonucleotide of any one of the preceding Embodiments, comprising optionally protected nucleoside [z2c3In].
1216. The oligonucleotide of any one of the preceding Embodiments, comprising optionally protected nucleoside [z5C].
1217. The oligonucleotide of any one of the preceding Embodiments, comprising optionally protected nucleoside Asm01.
1218. The oligonucleotide of any one of the preceding Embodiments, comprising optionally protected nucleoside Gsm01.
1219. The oligonucleotide of any one of the preceding Embodiments, comprising optionally protected nucleoside 5MSfC.
1220. The oligonucleotide of any one of the preceding Embodiments, comprising optionally protected nucleoside Usm04.
1221. The oligonucleotide of any one of the preceding Embodiments, comprising optionally protected nucleoside 5MRdT.
1222. The oligonucleotide of any one of the preceding Embodiments, comprising optionally protected nucleoside Csm04.
1223. The oligonucleotide of any one of the preceding Embodiments, comprising optionally protected nucleoside Csm11.
1224. The oligonucleotide of any one of the preceding Embodiments, comprising optionally protected nucleoside Gsm11.
1225. The oligonucleotide of any one of the preceding Embodiments, comprising optionally protected nucleoside Tsm11.
1226. The oligonucleotide of any one of the preceding Embodiments, comprising optionally protected nucleoside b009Csm11.
1227. The oligonucleotide of any one of the preceding Embodiments, comprising optionally protected nucleoside b009Csm12.
1228. The oligonucleotide of any one of the preceding Embodiments, comprising optionally protected nucleoside Gsm12.
1229. The oligonucleotide of any one of the preceding Embodiments, comprising optionally protected nucleoside Tsm12.
1230. The oligonucleotide of any one of the preceding Embodiments, comprising optionally protected nucleoside Csm12.
1231. The oligonucleotide of any one of the preceding Embodiments, comprising optionally protected nucleoside rCsm13.
1232. The oligonucleotide of any one of the preceding Embodiments, comprising optionally protected nucleoside rCsm14.
1233. The oligonucleotide of any one of the preceding Embodiments, comprising optionally protected nucleoside Csm15.
1234. The oligonucleotide of any one of the preceding Embodiments, comprising optionally protected nucleoside b001Asm15.
1235. The oligonucleotide of any one of the preceding Embodiments, comprising optionally protected nucleoside b008Usm15.
1236. The oligonucleotide of any one of the preceding Embodiments, comprising optionally protected nucleoside Csm16.
1237. The oligonucleotide of any one of the preceding Embodiments, comprising optionally protected nucleoside Csm17.
1238. The oligonucleotide of any one of the preceding Embodiments, comprising optionally protected nucleoside b008Usm19.
1239. The oligonucleotide of any one of the preceding Embodiments, comprising optionally protected nucleoside Csm19.
1240. The oligonucleotide of any one of the preceding Embodiments, comprising optionally protected abasic nucleoside.
1241. The oligonucleotide of any one of the preceding Embodiments, comprising optionally protected L010.
1242. The oligonucleotide of any one of the preceding Embodiments, comprising optionally protected nucleoside L034.
1243. The oligonucleotide of any one of the preceding Embodiments, comprising optionally protected nucleoside zdnp.
1244. The oligonucleotide of any one of the preceding Embodiments, comprising optionally protected nucleoside Tsm18.
1245. The oligonucleotide of any one of the preceding Embodiments, wherein each optionally protected nucleobase or nucleoside is independently an optionally substituted nucleobase or nucleoside, respectively.
1246. The oligonucleotide of any one of the preceding Embodiments, wherein each optionally protected or substituted nucleobase or nucleoside is not protected or substituted, respectively.
1247. The oligonucleotide of any one of the preceding Embodiments, comprising an internucleotidic linkage having the structure of -Y—P(═W)(—X—R^{L})-Z—
1248. The oligonucleotide of Embodiment 1247, wherein W is O.
1249. The oligonucleotide of Embodiment 1247, wherein W is S.
1250. The oligonucleotide of any one of Embodiments 1247-1249, wherein Y is —O—.
1251. The oligonucleotide of any one of Embodiments 1247-1250, wherein Z is a covalent bond.
1252. The oligonucleotide of any one of Embodiments 1247-1250, wherein Z is —O—.
1253. The oligonucleotide of any one of the preceding Embodiments, comprising an internucleotidic linkage comprising —X—R^{L}.
1254. The oligonucleotide of any one of the preceding Embodiments, comprising an internucleotidic linkage comprising —X—R^{L}.
1255. The oligonucleotide of any one of Embodiments 1247-1254, wherein —X—R^{L} is —N(R′)SO₂R″, wherein R″ is R′, —OR′, or —N(R′)₂.
1256. The oligonucleotide of any one of Embodiments 1247-1254, wherein —X—R^{L} is —NHSO₂R″, wherein R″ is optionally substituted C₁₋₆ aliphatic.
1257. The oligonucleotide of any one of Embodiments 1247-1254, wherein —X—R^{L} is —NHSO₂R″, wherein R″ is methyl.
1258. The oligonucleotide of any one of Embodiments 1247-1254, wherein —X—R^{L} is —NHSO₂R″, wherein R″ is optionally substituted phenyl.
1259. The oligonucleotide of any one of the preceding Embodiments, comprising n002.
1260. The oligonucleotide of any one of the preceding Embodiments, comprising n006.
1261. The oligonucleotide of any one of the preceding Embodiments, comprising n020.
1262. The oligonucleotide of any one of the preceding Embodiments, comprising —OP(═O)(NHSO₂CH₃)O—.
1263. The oligonucleotide of any one of the preceding Embodiments, wherein the first one, two, or three internucleotidic linkages are each independently an internucleotidic linkage of any one of Embodiments 1255-1262.
1264. The oligonucleotide of any one of the preceding Embodiments, wherein the last one, two, or three internucleotidic linkages are each independently an internucleotidic linkage of any one of Embodiments 1255-1262.
1265. The oligonucleotide of any one of the preceding Embodiments, wherein one or more internal internucleotidic linkages are each independently an internucleotidic linkage of any one of Embodiments 1255-1262.
1266. The oligonucleotide of any one of Embodiments 1247-1254, wherein —X—R^{L} is —N(R′)C(O)R″, wherein R″ is R′, —OR′, or —N(R′)₂.
1267. The oligonucleotide of any one of Embodiments 1247-1254, wherein —X—R^{L} is —NHC(O)R″, wherein R″ is optionally substituted C₁₋₆ aliphatic.
1268. The oligonucleotide of any one of Embodiments 1247-1254, wherein —X—R^{L} is —NHC(O)R″, wherein R″ is methyl.
1269. The oligonucleotide of any one of Embodiments 1247-1254, wherein —X—R^{L} is —NHC(O)R″, wherein R″ is optionally substituted phenyl.
1270. The oligonucleotide of any one of Embodiments 1247-1254, wherein —X—R^{L} is —NHC(O)R″, wherein R″ is —OR′.
1271. The oligonucleotide of any one of Embodiments 1247-1254, wherein —X—R^{L} is —NHC(O)R″, wherein R″ is —N(R′)₂.
1272. The oligonucleotide of any one of Embodiments 1247-1254, wherein —X—R^{L} is —N(R′)P(O)(R″)₂, wherein each R″ is independently R′, —OR′, or —N(R′)₂.
1273. The oligonucleotide of any one of Embodiments 1247-1254, wherein —X—R^{L} is —N(R′)P(S)(R″)₂, wherein each R″ is independently R′, —OR′, or —N(R′)₂.
1274. The oligonucleotide of any one of Embodiments 1247-1254, wherein —X—R^{L} is selected from Table L-1, L-2, L-3, L-4, L-5 or L-6.
1275. The oligonucleotide of any one of the preceding Embodiments, wherein about 20%-90% (e.g., about 20%-80%, 20%-70%, 30%-90%, 30%-80%, 30%-70%, 30%-60%, 30%-50%, about 30%, 40%, 50%, 60% or 70%) of all sugars of the oligonucleotide are 2′-F modified sugars.
1276. The oligonucleotide of any one of the preceding Embodiments, wherein about 30%-70% (e.g., about 30%-60%, 30%-50%, about 30%, 40%, 50%, 60% or 70%) of all sugars of the oligonucleotide are 2′-F modified sugars.
1277. The oligonucleotide of any one of the preceding Embodiments, wherein about 30%-60% (e.g., about 40%-60%, 30%-50%, about 30%, 40%, 50%, 60% or 70%) of all sugars of the oligonucleotide are 2′-F modified sugars.
1278. The oligonucleotide of any one of the preceding Embodiments, wherein at least about 65% of all sugars of the oligonucleotide are 2′-F modified sugars.
1279. The oligonucleotide of any one of the preceding Embodiments, wherein at least about 70% of all sugars of the oligonucleotide are 2′-F modified sugars.
1280. The oligonucleotide of any one of the preceding Embodiments, wherein at least about 75% of all sugars of the oligonucleotide are 2′-F modified sugars.
1281. The oligonucleotide of any one of the preceding Embodiments, wherein at least about 80% of all sugars of the oligonucleotide are 2′-F modified sugars.
1282. The oligonucleotide of any one of the preceding Embodiments, wherein at least about 85% of all sugars of the oligonucleotide are 2′-F modified sugars.
1283. The oligonucleotide of any one of the preceding Embodiments, wherein at least about 90% of all sugars of the oligonucleotide are 2′-F modified sugars.
1284. The oligonucleotide of any one of the preceding Embodiments, wherein about 20%-90% (e.g., about 20%-80%, 20%-70%, 30%-90%, 30%-80%, 30%-70%, 30%-60%, 30%-50%, about 30%, 40%, 50%, 60% or 70%) of all sugars of the oligonucleotide are each independently 2′-OR modified sugars wherein R is not -H.
1285. The oligonucleotide of any one of the preceding Embodiments, wherein about 30%-70% (e.g., about 30%-60%, 30%-50%, about 30%, 40%, 50%, 60% or 70%) of all sugars of the oligonucleotide are each independently 2′-OR modified sugars wherein R is not —H.
1286. The oligonucleotide of any one of the preceding Embodiments, wherein about 30%-60% (e.g., about 40%-60%, 30%-50%, about 30%, 40%, 50%, 60% or 70%) of all sugars of the oligonucleotide are each independently 2′-OR modified sugars wherein R is not —H.
1287. The oligonucleotide of any one of Embodiments 1284-1286, wherein a 2′-OR modified sugar is a 2′-OR modified sugar wherein R is optionally substituted C₁₋₆ aliphatic.
1288. The oligonucleotide of any one of the preceding Embodiments, wherein a 2′-OR modified sugar is a 2′-OMe modified sugar.
1289. The oligonucleotide of any one of the preceding Embodiments, wherein a 2′-OR modified sugar is a 2′-MOE modified sugar.
1290. The oligonucleotide of any one of the preceding Embodiments, wherein a 2′-OR modified sugar is a bicyclic sugar.
1291. The oligonucleotide of any one of the preceding Embodiments, wherein a 2′-OR modified sugar is a LNA sugar.
1292. The oligonucleotide of any one of the preceding Embodiments, wherein a 2′-OR modified sugar is a cEt sugar.
1293. The oligonucleotide of any one of Embodiments 1284-1286, wherein each 2′-OR modified sugar is independently a 2′-OR modified sugar wherein R is optionally substituted C₁₋₆ aliphatic.
1294. The oligonucleotide of any one of Embodiments 1284-1286, wherein each 2′-OR modified sugar is independently a 2′-OMe or 2′-MOE modified sugar.
1295. The oligonucleotide of any one of Embodiments 1284-1286, wherein each 2′-OR modified sugar is independently a 2′-OMe or 2′-MOE modified sugar, wherein at least one is a 2′-OMe modified sugar and at least one is a 2′-MOE modified sugar.
1296. The oligonucleotide of any one of Embodiments 1284-1286, wherein each 2′-OR modified sugar is a 2′-OMe modified sugar.
1297. The oligonucleotide of any one of the preceding Embodiments, wherein the first domain comprises one or more (e.g., 1-20, 1-15, 1-14, 1-13, 1-12, 1-11, 1-10, 2-20, 3-15. 4-15, 5-15, 1, 2, 3, 4, 5, 6, 7, 8, 9, 10, 11, 12, 13, 14, 15, 16, 17, 18, 19, 20, etc.) 2′-F blocks and one or more (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) separating blocks, wherein each sugar in each 2′-F block is independently a 2′-F modified sugar, and wherein each sugar in each separating block is independently a sugar other than a 2′-F modified sugar.
1298. The oligonucleotide of any one of the preceding Embodiments, wherein there are 2 or more 2′-F blocks in the first domain.
1299. The oligonucleotide of any one of the preceding Embodiments, wherein there are 3 or more 2′-F blocks in the first domain.
1300. The oligonucleotide of any one of the preceding Embodiments, wherein there are 4 or more 2′-F blocks in the first domain.
1301. The oligonucleotide of any one of the preceding Embodiments, wherein there are 5 or more 2′-F blocks in the first domain.
1302. The oligonucleotide of any one of the preceding Embodiments, wherein there are 2 or more separating blocks in the first domain.
1303. The oligonucleotide of any one of the preceding Embodiments, wherein there are 3 or more separating blocks in the first domain.
1304. The oligonucleotide of any one of the preceding Embodiments, wherein there are 4 or more separating blocks in the first domain.
1305. The oligonucleotide of any one of the preceding Embodiments, wherein there are 5 or more separating blocks in the first domain.
1306. The oligonucleotide of any one of the preceding Embodiments, wherein each sugar in each separating block is independently a 2′-modified sugar.
1307. The oligonucleotide of any one of the preceding Embodiments, wherein a sugar in a separating block is independently a 2′-OR sugar wherein R is not —H.
1308. The oligonucleotide of any one of the preceding Embodiments, wherein each separating block independently comprises a 2′-OR modified sugar wherein R is not —H.
1309. The oligonucleotide of any one of the preceding Embodiments, wherein a sugar in a separating block is independently a 2′-OR sugar wherein R is optionally substituted C₁₋₆ aliphatic.
1310. The oligonucleotide of any one of the preceding Embodiments, wherein each separating block independently comprises a 2′-OR modified sugar wherein R optionally substituted C₁₋₆ aliphatic.
1311. The oligonucleotide of any one of the preceding Embodiments, wherein each sugar in each separating block is independently a 2′-OR modified sugar or a bicyclic sugar, wherein R is optionally substituted C₁₋₆ aliphatic.
1312. The oligonucleotide of any one of the preceding Embodiments, wherein each sugar in a separating block is independently a 2′-OR modified sugar wherein R is optionally substituted C₁₋₆ aliphatic.
1313. The oligonucleotide of any one of the preceding Embodiments, wherein each sugar in each separating block is independently a 2′-OR modified sugar wherein R is optionally substituted C_{L-6} aliphatic.
1314. The oligonucleotide of any one of the preceding Embodiments, wherein each sugar in a separating block is independently a 2′-OMe or 2′-MOE modified sugar.
1315. The oligonucleotide of any one of the preceding Embodiments, wherein each sugar in each separating block is independently a 2′-OMe or 2′-MOE modified sugar.
1316. The oligonucleotide of any one of the preceding Embodiments, wherein a sugar in a separating block is a 2′-OME modified sugar.
1317. The oligonucleotide of any one of the preceding Embodiments, wherein each sugar in a separating block is independently a 2′-OMe modified sugar.
1318. The oligonucleotide of any one of the preceding Embodiments, wherein each sugar in a separating block is independently a 2′-MOE modified sugar.
1319. The oligonucleotide of any one of Embodiments 1-1305, wherein each sugar in each separating block is independently a 2′-OMe modified sugar.
1320. The oligonucleotide of any one of Embodiments 1-1305, wherein each sugar in each separating block is independently a 2′-MOE modified sugar.
1321. The oligonucleotide of any one of Embodiments 1297-1320, wherein in each 2′-F block there are independently about 1-20 (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12. 13, 14, 15, 16, 17, 18, 19, 20, etc.) 2′-F modified sugars.
1322. The oligonucleotide of any one of Embodiments 1297-1320, wherein in each 2′-F block there are about 1-10, e.g., 1, 2, 3, 4, 5, 6, 7, 8, 9, or 10, 2′-F modified sugars.
1323. The oligonucleotide of any one of Embodiments 1297-1320, wherein in each 2′-F block there are about 1, 2, 3, 4 or 5 2′-F modified sugars.
1324. The oligonucleotide of any one of Embodiments 1297-1320, wherein in each 2′-F block there are about 1, 2, or 3 2′-F modified sugars.
1325. The oligonucleotide of any one of Embodiments 1297-1324, wherein in each separating block there are independently about 1-20 (e.g., 1-20, 1-15, 1-14, 1-13, 1-12, 1-11, 1-10, 2-20, 3-15, 4-15, 5-15, 1, 2, 3, 4, 5, 6, 7, 8, 9, 10, 11, 12, 13, 14, 15, 16, 17, 18, 19, 20, etc.) sugars.
1326. The oligonucleotide of any one of Embodiments 1297-1324, wherein in each separating block there are about 1-10, e.g., 1, 2, 3, 4, 5, 6, 7, 8, 9, or 10 sugars.
1327. The oligonucleotide of any one of Embodiments 1297-1324, wherein in each separating block there are about 1, 2, 3, 4 or 5 sugars.
1328. The oligonucleotide of any one of Embodiments 1297-1324, wherein in each separating there are about 1, 2, or 3 sugars.
1329. The oligonucleotide of any one of Embodiments 1297-1328, wherein each block in a first domain that is bonded to a 2′-F block in a first domain is a separating block.
1330. The oligonucleotide of any one of Embodiments 1297-1329, wherein each block in a first domain that is bonded to a separating block in a first domain is a 2′-F block.
1331. The oligonucleotide of any one of the preceding Embodiments, wherein the oligonucleotide comprises two or more 2′-F modified sugar blocks, wherein each 2′-F modified sugar block independently comprises or consists of 2, 3, 4, 5, 6, 7, 8, 9, or 10 consecutive 2′-F modified sugars, wherein each two consecutive 2′-F modified sugar blocks are independently separated by a separating block which separating block comprises one or more sugars that are independently not 2′-F modified sugars and no consecutive 2′-F modified sugars.
1332. The oligonucleotide of any one of the preceding Embodiments, wherein at least 50%, 60%, 70%, 75%, 80%, 85%, 90%, 95% or 99% (e.g., 50%-100%, 60%-100%, 70-100%, 75%-100%, 80%-100%, 90%-100%, 95%-100%, 60%-95%, 70%-95%, 75-95%, 80-95%, 85-95%, 90-95%, 50%, 60%, 70%, 75%, 80%, 85%, 90%, 95%, or 99%, etc.) of all, or all phosphorothioate internucleotidic linkages, are Sp.
1333. The oligonucleotide of any one of the preceding Embodiments, wherein the first domain is at the 5′ side of a second domain.
1334. The oligonucleotide of any one of the preceding Embodiments, wherein the first domain is at the 3′ side of a second domain.
1335. The oligonucleotide of any one of the preceding Embodiments, wherein in the second domain the first subdomain is at the 5′ side of the second subdomain, and the third subdomain is at the 3′ side of the second subdomain.
1336. The oligonucleotide of any one ofthe preceding Embodiments, wherein the oligonucleotide comprises a 5′-N₁N₀N₋₁-3′, wherein each of N₋₁, N₀, and N₁ is independently a nucleoside.
1337. The oligonucleotide of any one of the preceding Embodiments, wherein the oligonucleotide comprises 5′-N₂N₁N₀N₋₁N₋₂-3′ wherein each of N₂, N₁, N₀, N₋₁, and N₋₂ is independently a nucleoside.
1338. The oligonucleotide of any one of the preceding Embodiments, wherein the oligonucleotide comprises 5′-N₃N₂N₁N₀N₋₁N₋₂N₋₃-3′ wherein each of N₃, N₂, N₁, N₀, N₋₁, N₋₂, and N₋₃ is independently a nucleoside.
1339. The oligonucleotide of any one of the preceding Embodiments, wherein the oligonucleotide comprises 5′-N₄N₃N₂N₁N₀N₋₁N₋₂N₋₃N₋₄-3′ wherein each of N₄, N₃, N₂, N₁, N₀, N₋₁, N₋₂, N₋₃, and N₋₄ is independently a nucleoside.
1340. The oligonucleotide of any one of the preceding Embodiments, wherein the oligonucleotide comprises 5′-N₅N₄N₃N₂N₁N₀N₋₁N₋₂N₋₃N₋₄N₋₅-3′ wherein each of N₅, N₄, N₃, N₂, N₁, N₀, N₋₁, N₋₂, N₋₃, N₋₄, and N₋₅ is independently a nucleoside.
1341. The oligonucleotide of any one of the preceding Embodiments, wherein the oligonucleotide comprises 5′-N₆N₅N₄N₃N₂N₁N₀N₋₁N₋₂N₋₃N₋₄N₋₅N₋₆-3′ wherein each of N₆, N₅, N₄, N₃, N₂, N₁, N₀, N₋₁, N₋₂, N₋₃, N₋₄, N₋₅, and N₋₆ is independently a nucleoside.
1342. The oligonucleotide of any one of the preceding Embodiments, wherein the second subdomain comprises a 5′-N₁N₀N₋₁-3′, wherein each of N₋₁, N₀, and N₁ is independently a nucleoside.
1343. The oligonucleotide of any one of the preceding Embodiments, wherein when the oligonucleotide is aligned with a target nucleic acid, N₀ is opposite to a target adenosine.
1344. The oligonucleotide of any one of the preceding Embodiments, wherein each of N₋₁, N₀, and N₁ independently has a 2′-F modified sugar, a natural RNA sugar, or a sugar having no 2′-substituent replacing 2′-OH of a natural RNA sugar.
1345. The oligonucleotide of any one of the preceding Embodiments, wherein each of N₋₁, N₀, and N₁ independently has a 2′-F modified sugar, a natural RNA sugar, or a sugar having no 2′-substituents.
1346. The oligonucleotide of any one of the preceding Embodiments, wherein each of N₋₁, N₀, and N₁ independently has a 2′-F modified sugar, a natural RNA sugar, or a natural DNA sugar.
1347. The oligonucleotide of any one of the preceding Embodiments, wherein no more than one of N₋₁, N₀, and N₁ has a 2′-F modified sugar.
1348. The oligonucleotide of any one of the preceding Embodiments, wherein no more than one of N₋₁, N₀, and N₁ has a natural RNA sugar.
1349. The oligonucleotide of any one of the preceding Embodiments, wherein the sugar of N₁ is a 2′-F modified sugar.
1350. The oligonucleotide of any one of Embodiments 1-1348, wherein the sugar of N₀ is a sugar comprising no substituent at a position corresponding to 2′-OH of a natural RNA sugar.
1351. The oligonucleotide of any one of Embodiments 1-1348, wherein the sugar of N₀ is a sugar comprising no 2′-substituent.
1352. The oligonucleotide of any one of Embodiments 1-1348, wherein the sugar of N₁ is sm19.
1353. The oligonucleotide of any one of Embodiments 1-1348, wherein the sugar of N₁ is a natural DNA sugar.
1354. The oligonucleotide of any one of Embodiments 1-1348, wherein the sugar of N₁ is a natural RNA sugar.
1355. The oligonucleotide of any one of the preceding Embodiments, wherein the sugar of N₀ is a modified sugar.
1356. The oligonucleotide of any one of the preceding Embodiments, wherein the sugar of N₀ is a 2′-F modified sugar.
1357. The oligonucleotide of any one of Embodiments 1-1354, wherein the sugar of N₀ is a 2′-OR modified sugar, wherein R is optionally substituted C₁₋₆ alkyl.
1358. The oligonucleotide of any one of Embodiments 1-1354, wherein the sugar of N₀ is a 2′-OMe modified sugar.
1359. The oligonucleotide of any one of Embodiments 1-1354, wherein the sugar of N₀ is a 2′-MOE modified sugar.
1360. The oligonucleotide of any one of Embodiments 1-1354, wherein the sugar of N₀ is a sugar comprising no substituent at a position corresponding to 2′-OH of a natural RNA sugar.
1361. The oligonucleotide of any one of Embodiments 1-1354, wherein the sugar of N₀ is a sugar comprising no 2′-substituent.
1362. The oligonucleotide of any one of Embodiments 1-1354, wherein the sugar of N₀ is a 5′-modified sugar.
1363. The oligonucleotide of any one of Embodiments 1-1354, wherein the sugar of N₀ is a 5′-Me modified sugar.
1364. The oligonucleotide of any one of Embodiments 1-1354, wherein the sugar of N₀ is a non-cyclic sugar.
1365. The oligonucleotide of any one of Embodiments 1-1354, wherein the sugar of N₀ is sm01.
1366. The oligonucleotide of any one of Embodiments 1-1354, wherein the sugar of N₀ is sml5.
1367. The oligonucleotide of any one of Embodiments 1-1354, wherein the sugar of N₀ is sm19.
1368. The oligonucleotide of any one of Embodiments 1-1354, wherein the sugar of N₀ is a sugar is a substituted natural DNA sugar one of whose 2′-H is substituted with —OH or —F and the other 2′-H is not substituted.
1369. The oligonucleotide of any one of Embodiments 1-1354, wherein the sugar of N₀ is a natural DNA sugar.
1370. The oligonucleotide of any one of Embodiments 1-1354, wherein the sugar of N₀ is the sugar of any one of Embodiments 820-852.
1371. The oligonucleotide of any one of Embodiments 1-1354, wherein the sugar of N₀ is a natural RNA sugar.
1372. The oligonucleotide of any one of Embodiments 1-1371, wherein the nucleobase of N₀ is C.
1373. The oligonucleotide of any one of Embodiments 1-1371, wherein the nucleobase of N₀ is hypoxanthine.
1374. The oligonucleotide of any one of Embodiments 1-1371, wherein the nucleobase of N₀ is T.
1375. The oligonucleotide of any one of Embodiments 1-1371, wherein the nucleobase of N₀ is A.
1376. The oligonucleotide of any one of Embodiments 1-1371, wherein the nucleobase of N₀ is G.
1377. The oligonucleotide of any one of Embodiments 1-1371, wherein the nucleobase of N₀ is U.
1378. The oligonucleotide of any one of Embodiments 1-1371, wherein the nucleobase of N₀ is b001U.
1379. The oligonucleotide of any one of Embodiments 1-1371, wherein the nucleobase of N₀ is b002U.
1380. The oligonucleotide of any one of Embodiments 1-1371, wherein the nucleobase of N₀ is b003U.
1381. The oligonucleotide of any one of Embodiments 1-1371, wherein the nucleobase of N₀ is b004U.
1382. The oligonucleotide of any one of Embodiments 1-1371, wherein the nucleobase of N₀ is b005U.
1383. The oligonucleotide of any one of Embodiments 1-1371, wherein the nucleobase of N₀ is b006U.
1384. The oligonucleotide of any one of Embodiments 1-1371, wherein the nucleobase of N₀ is b007U.
1385. The oligonucleotide of any one of Embodiments 1-1371, wherein the nucleobase of N₀ is b008U.
1386. The oligonucleotide of any one of Embodiments 1-1371, wherein the nucleobase of N₀ is b009U.
1387. The oligonucleotide of any one of Embodiments 1-1371, wherein the nucleobase of N₀ is b011U.
1388. The oligonucleotide of any one of Embodiments 1-1371, wherein the nucleobase of N₀ is b012U.
1389. The oligonucleotide of any one of Embodiments 1-1371, wherein the nucleobase of N₀ is b013U.
1390. The oligonucleotide of any one of Embodiments 1-1371, wherein the nucleobase of N₀ is b014U.
1391. The oligonucleotide of any one of Embodiments 1-1371, wherein the nucleobase of N₀ is b015U.
1392. The oligonucleotide of any one of Embodiments 1-1371, wherein the nucleobase of N₀ is b001A.
1393. The oligonucleotide of any one of Embodiments 1-1371, wherein the nucleobase of N₀ is b002A.
1394. The oligonucleotide of any one of Embodiments 1-1371, wherein the nucleobase of N₀ is b003A.
1395. The oligonucleotide of any one of Embodiments 1-1371, wherein the nucleobase of N₀ is b004A.
1396. The oligonucleotide of any one of Embodiments 1-1371, wherein the nucleobase of N₀ is b005A.
1397. The oligonucleotide of any one of Embodiments 1-1371, wherein the nucleobase of N₀ is b006A.
1398. The oligonucleotide of any one of Embodiments 1-1371, wherein the nucleobase of N₀ is b007A.
1399. The oligonucleotide of any one of Embodiments 1-1371, wherein the nucleobase of N₀ is b001G.
1400. The oligonucleotide of any one of Embodiments 1-1371, wherein the nucleobase of N₀ is b002G.
1401. The oligonucleotide of any one of Embodiments 1-1371, wherein the nucleobase of N₀ is b001C.
1402. The oligonucleotide of any one of Embodiments 1-1371, wherein the nucleobase of N₀ is b002C.
1403. The oligonucleotide of any one of Embodiments 1-1371, wherein the nucleobase of N₀ is b003C.
1404. The oligonucleotide of any one of Embodiments 1-1371, wherein the nucleobase of N₀ is b004C.
1405. The oligonucleotide of any one of Embodiments 1-1371, wherein the nucleobase of N₀ is b005C.
1406. The oligonucleotide of any one of Embodiments 1-1371, wherein the nucleobase of N₀ is b006C.
1407. The oligonucleotide of any one of Embodiments 1-1371, wherein the nucleobase of N₀ is b007C.
1408. The oligonucleotide of any one of Embodiments 1-1371, wherein the nucleobase of N₀ is b008C.
1409. The oligonucleotide of any one of Embodiments 1-1371, wherein the nucleobase of N₀ is b009C.
1410. The oligonucleotide of any one of Embodiments 1-1371, wherein the nucleobase of N₀ is b002I.
1411. The oligonucleotide of any one of Embodiments 1-1371, wherein the nucleobase of N₀ is b003I.
1412. The oligonucleotide of any one of Embodiments 1-1371, wherein the nucleobase of N₀ is b004I.
1413. The oligonucleotide of any one of Embodiments 1-1371, wherein the nucleobase of N₀ is b014I.
1414. The oligonucleotide of any one of Embodiments 1-1371, wherein the nucleobase of N₀ is [3ne5U].
1415. The oligonucleotide of any one of Embodiments 1-1371, wherein the nucleobase of N₀ is [3nT].
1416. The oligonucleotide of any one of Embodiments 1-1371, wherein the nucleobase of N₀ is [3nfl5U].
1417. The oligonucleotide of any one of Embodiments 1-1371, wherein the nucleobase of N₀ is [3npry5U].
1418. The oligonucleotide of any one of Embodiments 1-1371, wherein the nucleobase of N₀ is [3ncn5U].
1419. The oligonucleotide of any one of Embodiments 1-1371, wherein the nucleobase of N₀ is [napth6o8A].
1420. The oligonucleotide of any one of Embodiments 1-1371, wherein the nucleobase of N₀ is [ipr6o8A].
1421. The oligonucleotide of any one of Embodiments 1-1371, wherein the nucleobase of N₀ is [c7In].
1422. The oligonucleotide of any one of Embodiments 1-1371, wherein the nucleobase of N₀ is [c39z48In].
1423. The oligonucleotide of any one of Embodiments 1-1371, wherein the nucleobase of N₀ is [z2c3In].
1424. The oligonucleotide of any one of Embodiments 1-1371, wherein the nucleobase of N₀ is [z5C].
1425. The oligonucleotide of any one of Embodiments 1-1371, wherein the nucleobase of N₀ is selected from Table BA-1.
1426. The oligonucleotide of any one of Embodiments 1-1371, wherein the nucleobase of N₀ is zndp.
1427. The oligonucleotide of any one of Embodiments 1-1354, wherein N₀ is dC.
1428. The oligonucleotide of any one of Embodiments 1-1354, wherein N₀ is fU.
1429. The oligonucleotide of any one of Embodiments 1-1354, wherein N₀ is dU.
1430. The oligonucleotide of any one of Embodiments 1-1354, wherein N₀ is fA.
1431. The oligonucleotide of any one of Embodiments 1-1354, wherein N₀ is dA.
1432. The oligonucleotide of any one of Embodiments 1-1354, wherein N₀ is fT.
1433. The oligonucleotide of any one of Embodiments 1-1354, wherein N₀ is dT.
1434. The oligonucleotide of any one of Embodiments 1-1354, wherein N₀ is fC.
1435. The oligonucleotide of any one of Embodiments 1-1354, wherein N₀ is fG.
1436. The oligonucleotide of any one of Embodiments 1-1354, wherein N₀ is dG.
1437. The oligonucleotide of any one of Embodiments 1-1354, wherein N₀ is dI.
1438. The oligonucleotide of any one of Embodiments 1-1354, wherein N₀ is fI.
1439. The oligonucleotide of any one of Embodiments 1-1354, wherein N₀ is aC.
1440. The oligonucleotide of any one of Embodiments 1-1354, wherein N₀ is m5dC.
1441. The oligonucleotide of any one of Embodiments 1-1354, wherein N₀ is 5MRm5dC.
1442. The oligonucleotide of any one of Embodiments 1-1354, wherein N₀ is 5MSm5dC.
1443. The oligonucleotide of any one of Embodiments 1-1354, wherein N₀ is b001G.
1444. The oligonucleotide of any one of Embodiments 1-1354, wherein N₀ is b002G.
1445. The oligonucleotide of any one of Embodiments 1-1354, wherein N₀ is b001C.
1446. The oligonucleotide of any one of Embodiments 1-1354, wherein N₀ is b002C.
1447. The oligonucleotide of any one of Embodiments 1-1354, wherein N₀ is b003C.
1448. The oligonucleotide of any one of Embodiments 1-1354, wherein N₀ is b003mC.
1449. The oligonucleotide of any one of Embodiments 1-1354, wherein N₀ is b004C.
1450. The oligonucleotide of any one of Embodiments 1-1354, wherein N₀ is b005C.
1451. The oligonucleotide of any one of Embodiments 1-1354, wherein N₀ is b006C.
1452. The oligonucleotide of any one of Embodiments 1-1354, wherein N₀ is b007C.
1453. The oligonucleotide of any one of Embodiments 1-1354, wherein N₀ is b008C.
1454. The oligonucleotide of any one of Embodiments 1-1354, wherein N₀ is b009C.
1455. The oligonucleotide of any one of Embodiments 1-1354, wherein N₀ is b001A.
1456. The oligonucleotide of any one of Embodiments 1-1354, wherein N₀ is b002A.
1457. The oligonucleotide of any one of Embodiments 1-1354, wherein N₀ is b003A.
1458. The oligonucleotide of any one of Embodiments 1-1354, wherein N₀ is b004A.
1459. The oligonucleotide of any one of Embodiments 1-1354, wherein N₀ is b005A.
1460. The oligonucleotide of any one of Embodiments 1-1354, wherein N₀ is b006A.
1461. The oligonucleotide of any one of Embodiments 1-1354, wherein N₀ is b007A.
1462. The oligonucleotide of any one of Embodiments 1-1354, wherein N₀ is b001U.
1463. The oligonucleotide of any one of Embodiments 1-1354, wherein N₀ is b002U.
1464. The oligonucleotide of any one of Embodiments 1-1354, wherein N₀ is b003U.
1465. The oligonucleotide of any one of Embodiments 1-1354, wherein N₀ is b004U.
1466. The oligonucleotide of any one of Embodiments 1-1354, wherein N₀ is b005U.
1467. The oligonucleotide of any one of Embodiments 1-1354, wherein N₀ is b006U.
1468. The oligonucleotide of any one of Embodiments 1-1354, wherein N₀ is b007U.
1469. The oligonucleotide of any one of Embodiments 1-1354, wherein N₀ is b008U.
1470. The oligonucleotide of any one of Embodiments 1-1354, wherein N₀ is b009U.
1471. The oligonucleotide of any one of Embodiments 1-1354, wherein N₀ is b010U.
1472. The oligonucleotide of any one of Embodiments 1-1354, wherein N₀ is b011U.
1473. The oligonucleotide of any one of Embodiments 1-1354, wherein N₀ is b012U.
1474. The oligonucleotide of any one of Embodiments 1-1354, wherein N₀ is b013U.
1475. The oligonucleotide of any one of Embodiments 1-1354, wherein N₀ is b014U.
1476. The oligonucleotide of any one of Embodiments 1-1354, wherein N₀ is b015U.
1477. The oligonucleotide of any one of Embodiments 1-1354, wherein N₀ is b002I.
1478. The oligonucleotide of any one of Embodiments 1-1354, wherein N₀ is b0031.
1479. The oligonucleotide of any one of Embodiments 1-1354, wherein N₀ is b004I.
1480. The oligonucleotide of any one of Embodiments 1-1354, wherein N₀ is b014I.
1481. The oligonucleotide of any one of Embodiments 1-1354, wherein N₀ is [3nT].
1482. The oligonucleotide of any one of Embodiments 1-1354, wherein N₀ is [3ne5U].
1483. The oligonucleotide of any one of Embodiments 1-1354, wherein N₀ is [3nfl5U].
1484. The oligonucleotide of any one of Embodiments 1-1354, wherein N₀ is [3npry5U].
1485. The oligonucleotide of any one of Embodiments 1-1354, wherein N₀ is [3ncn5U].
1486. The oligonucleotide of any one of Embodiments 1-1354, wherein N₀ is [napth6o8A].
1487. The oligonucleotide of any one of Embodiments 1-1354, wherein N₀ is [ipr6o8A].
1488. The oligonucleotide of any one of Embodiments 1-1354, wherein N₀ is [c7In].
1489. The oligonucleotide of any one of Embodiments 1-1354, wherein N₀ is [c39z481n].
1490. The oligonucleotide of any one of Embodiments 1-1354, wherein N₀ is [z2c3In].
1491. The oligonucleotide of any one of Embodiments 1-1354, wherein N₀ is [z5C].
1492. The oligonucleotide of any one of Embodiments 1-1354, wherein N₀ is Asm01.
1493. The oligonucleotide of any one of Embodiments 1-1354, wherein N₀ is Gsm01.
1494. The oligonucleotide of any one of Embodiments 1-1354, wherein N₀ is Tsm01.
1495. The oligonucleotide of any one of Embodiments 1-1354, wherein N₀ is 5MSfC.
1496. The oligonucleotide of any one of Embodiments 1-1354, wherein N₀ is Usm04.
1497. The oligonucleotide of any one of Embodiments 1-1354, wherein N₀ is 5MRdT.
1498. The oligonucleotide of any one of Embodiments 1-1354, wherein N₀ is Csm04.
1499. The oligonucleotide of any one of Embodiments 1-1354, wherein N₀ is Csm11. 1500. The oligonucleotide of any one of Embodiments 1-1354, wherein N₀ is Gsm11.
1501. The oligonucleotide of any one of Embodiments 1-1354, wherein N₀ is Tsm11.
1502. The oligonucleotide of any one of Embodiments 1-1354, wherein N₀ is b009Csm11.
1503. The oligonucleotide of any one of Embodiments 1-1354, wherein N₀ is b009Csm12.
1504. The oligonucleotide of any one of Embodiments 1-1354, wherein N₀ is Gsm12.
1505. The oligonucleotide of any one of Embodiments 1-1354, wherein N₀ is Tsm12.
1506. The oligonucleotide of any one of Embodiments 1-1354, wherein N₀ is Csm12.
1507. The oligonucleotide of any one of Embodiments 1-1354, wherein N₀ is rCsm13.
1508. The oligonucleotide of any one of Embodiments 1-1354, wherein N₀ is rCsm14.
1509. The oligonucleotide of any one of Embodiments 1-1354, wherein N₀ is Csm15.
1510. The oligonucleotide of any one of Embodiments 1-1354, wherein N₀ is b001Asm15.
1511. The oligonucleotide of any one of Embodiments 1-1354, wherein N₀ is b008Usm15.
1512. The oligonucleotide of any one of Embodiments 1-1354, wherein N₀ is Csm16.
1513. The oligonucleotide of any one of Embodiments 1-1354, wherein N₀ is Csm17.
1514. The oligonucleotide of any one of Embodiments 1-1354, wherein N₀ is b008Usm19.
1515. The oligonucleotide of any one of Embodiments 1-1354, wherein N₀ is Csm19.
1516. The oligonucleotide of any one of Embodiments 1-1354, wherein N₀ is abasic.
1517. The oligonucleotide of any one of Embodiments 1-1354, wherein N₀ is L010.
1518. The oligonucleotide of any one of Embodiments 1-1354, wherein N₀ is L034.
1519. The oligonucleotide of any one of Embodiments 1-1354, wherein N₀ is Csm15.
1520. The oligonucleotide of any one of Embodiments 1-1354, wherein N₀ is Tsm18.
1521. The oligonucleotide of any one of Embodiments 1-1354, wherein N₀ is b001rA.
1522. The oligonucleotide of any one of the preceding Embodiments, wherein nucleobase of N₁ is A, T, C, G, U, hypoxanthine, b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b003I, b004I, b014I, [3nT], [3ne5U], [3nfl5U], [3npry5U], [3ncn5U], [napth6o8A], [ipr6o8A], [c7In], [c39z481n], [z2c3In], [z5C], or zdnp.
1523. The oligonucleotide of any one of the preceding Embodiments, wherein nucleobase of N₁ is a modified nucleobase.
1524. The oligonucleotide of any one of the preceding Embodiments, wherein the sugar of N₁ is a 2′-F modified sugar.
1525. The oligonucleotide of any one of Embodiments 1-1522, wherein the sugar of N₁ is a sugar comprising no substituent at a position corresponding to 2′-OH of a natural RNA sugar.
1526. The oligonucleotide of any one of Embodiments 1-1522, wherein the sugar of N₁ is a sugar comprising no 2′-substituent.
1527. The oligonucleotide of any one of Embodiments 1-1522, wherein the sugar of N₁ is a natural DNA sugar.
1528. The oligonucleotide of any one of Embodiments 1-1522, wherein the sugar of N₁ the sugar of any one of Embodiments 820-852.
1529. The oligonucleotide of any one of Embodiments 1-1522, wherein the sugar of N₁ is a natural RNA sugar.
1530. The oligonucleotide of any one of Embodiments 1-1522, where N₁ is dA, dT, dC, dG, dU, fA, fT, fC, fG or fU.
1531. The oligonucleotide of any one of Embodiments 1-1529, where the nucleobase of N₁ is A, T, C, G, U, hypoxanthine, b001U, b002U, b003U, b004U, b005U, b006U, b007U, b008U, b009U, b011U, b012U, b013U, b001A, b002A, b003A, b001G, b002G, b001C, b002C, b003C, b004C, b005C, b006C, b007C, b008C, b009C, b0021, b0031, b0041. b0141, [3nT], [3ne5U], [3nfl5U], [3npry5U], [3ncn5U], [napth6o8A], [ipr6o8A], [c7Tn], [c39z481n], [z2c3Tn], [z5C], or zdnp, or is selected from Table BA-1.
1532. The oligonucleotide of any one of Embodiments 1-1522, wherein N₁ is b001A.
1533. The oligonucleotide of any one of Embodiments 1-1522, wherein N₁ is b002A.
1534. The oligonucleotide of any one of Embodiments 1-1522, wherein N₁ is b003A.
1535. The oligonucleotide of any one of Embodiments 1-1522, wherein N₁ is b004A.
1536. The oligonucleotide of any one of Embodiments 1-1522, wherein N₁ is b005A.
1537. The oligonucleotide of any one of Embodiments 1-1522, wherein N₁ is b006A.
1538. The oligonucleotide of any one of Embodiments 1-1522, wherein N₁ is b007A.
1539. The oligonucleotide of any one of Embodiments 1-1522, wherein N₁ is b001C.
1540. The oligonucleotide of any one of Embodiments 1-1522, wherein N₁ is b004C.
1541. The oligonucleotide of any one of Embodiments 1-1522, wherein N₁ is b007C.
1542. The oligonucleotide of any one of Embodiments 1-1522, wherein N₁ is b008C.
1543. The oligonucleotide of any one of Embodiments 1-1522, wherein N₁ is b008U.
1544. The oligonucleotide of any one of Embodiments 1-1522, wherein N₁ is b010U.
1545. The oligonucleotide of any one of Embodiments 1-1522, wherein N₁ is b011U.
1546. The oligonucleotide of any one of Embodiments 1-1522, wherein N₁ is b012U.
1547. The oligonucleotide of any one of Embodiments 1-1522, wherein N₁ is b013U.
1548. The oligonucleotide of any one of Embodiments 1-1522, wherein N₁ is b014U.
1549. The oligonucleotide of any one of Embodiments 1-1522, wherein N₁ is b015U.
1550. The oligonucleotide of any one of Embodiments 1-1522, wherein N₁ is Csm11.
1551. The oligonucleotide of any one of Embodiments 1-1522, wherein N₁ is Csm12.
1552. The oligonucleotide of any one of Embodiments 1-1522, wherein N₁ is Csm17.
1553. The oligonucleotide of any one of Embodiments 1-1522, wherein N₁ is b009Csm11.
1554. The oligonucleotide of any one of Embodiments 1-1522, wherein N₁ is b009Csm12.
1555. The oligonucleotide of any one of Embodiments 1-1522, wherein N₁ is Gsm01.
1556. The oligonucleotide of any one of Embodiments 1-1522, wherein N₁ is Gsm11.
1557. The oligonucleotide of any one of Embodiments 1-1522, wherein N₁ is Gsm12.
1558. The oligonucleotide of any one of Embodiments 1-1522, wherein N₁ is TsmO1.
1559. The oligonucleotide of any one of Embodiments 1-1522, wherein N₁ is Tsm11.
1560. The oligonucleotide of any one of Embodiments 1-1522, wherein N₁ is Tsm12.
1561. The oligonucleotide of any one of Embodiments 1-1522, wherein N₁ is b001Asm15.
1562. The oligonucleotide of any one of Embodiments 1-1522, wherein N₁ is b008Usm15.
1563. The oligonucleotide of any one of Embodiments 1-1522, wherein N₁ is Tsm18.
1564. The oligonucleotide of any one of Embodiments 1-1522, wherein N₁ is Csm19.
1565. The oligonucleotide of any one of Embodiments 1-1522, wherein N₁ is b008Usm19.
1566. The oligonucleotide of any one of Embodiments 1-1522, wherein N₁ is L010.
1567. The oligonucleotide of any one of the preceding Embodiments, wherein the sugar of N₋₁ is a modified sugar.
1568. The oligonucleotide of any one of the preceding Embodiments, wherein the sugar of N₋₁ is a 2′-F modified sugar.
1569. The oligonucleotide of any one of Embodiments 1-1565, wherein the sugar of N₋₁ is a sugar comprising no substituent at a position corresponding to 2′-OH of a natural RNA sugar.
1570. The oligonucleotide of any one of Embodiments 1-1565, wherein the sugar of N₋₁ is a sugar comprising no 2′-substituent.
1571. The oligonucleotide of any one of Embodiments 1-1565, wherein the sugar of N₋₁ is sm19.
1572. The oligonucleotide of any one of Embodiments 1-1565, wherein the sugar of N₋₁ is a natural DNA sugar.
1573. The oligonucleotide of any one of Embodiments 1-1565, wherein the sugar of N₋₁ is a natural RNA sugar.
1574. The oligonucleotide of any one of the preceding Embodiments, wherein N₁ and N₋₁ are both complementary to their corresponding nucleosides when the oligonucleotide is aligned with a target nucleic acid.
1575. The oligonucleotide of any one of the preceding Embodiments, wherein at least one of N₁ and N₋₁ is independently produces a mismatch or a wobble base pairing when the oligonucleotide is aligned with a target nucleic acid.
1576. The oligonucleotide of Embodiment 1575, wherein the oligonucleotide provides comparable or higher editing levels of a target adenosine compared to a reference oligonucleotide, wherein the reference oligonucleotide is otherwise identical but has N₁ and N₋₁ that are complementary to their corresponding nucleosides when the reference oligonucleotide is aligned with the target nucleic acid, wherein the target adenosine is opposite to No when the oligonucleotide is aligned with the target nucleic acid.
1577. The oligonucleotide of any one of the preceding Embodiments, wherein the nucleobase of N₋₁ is A, T, C, G, U, hypoxanthine, b001U, b002U, b003U, b004U, b005U, b006U, b007U, b008U, b009U, b011U, b012U, b013U, b014U, b015U, b001A, b002A, b003A, b004A, b005A, b006A, b007A, b001G, b002G, b001C, b002C, b003C, b004C, b005C, b006C, b007C, b008C, b009C, b002I, b0031, b004I, b014I, [3nT], [3ne5U], [3nfl5U], [3npry5U], [3ncn5U], [napth6o8A], [ipr6o8A], [c7In], [c39z481n], [z2c3In], [z5C], or zdnp.
1578. The oligonucleotide of any one of the preceding Embodiments, wherein the nucleobase of N₋₁ is a modified nucleobase.
1579. The oligonucleotide of any one of Embodiments 1-1576, wherein the nucleobase of N₋₁ is hypoxanthine.
1580. The oligonucleotide of any one of Embodiments 1-1576, wherein the nucleobase of N₋₁ is C.
1581. The oligonucleotide of any one of Embodiments 1-1576, wherein the nucleobase of N₁ is T.
1582. The oligonucleotide of any one of Embodiments 1-1576, wherein the nucleobase of N₋₁ is A.
1583. The oligonucleotide of any one of Embodiments 1-1576, wherein the nucleobase of N₋₁ is G.
1584. The oligonucleotide of any one of Embodiments 1-1576, wherein the nucleobase of N₋₁ is U.
1585. The oligonucleotide of any one of Embodiments 1-1576, wherein the nucleobase of N₋₁ is b001U.
1586. The oligonucleotide of any one of Embodiments 1-1576, wherein the nucleobase of N₋₁ is b002U.
1587. The oligonucleotide of any one of Embodiments 1-1576, wherein the nucleobase of N₋₁ is b003U.
1588. The oligonucleotide of any one of Embodiments 1-1576, wherein the nucleobase of N₋₁ is b004U.
1589. The oligonucleotide of any one of Embodiments 1-1576, wherein the nucleobase of N₋₁ is b005U.
1590. The oligonucleotide of any one of Embodiments 1-1576, wherein the nucleobase of N₋₁ is b006U.
1591. The oligonucleotide of any one of Embodiments 1-1576, wherein the nucleobase of N₋₁ is b007U.
1592. The oligonucleotide of any one of Embodiments 1-1576, wherein the nucleobase of N₋₁ is b008U.
1593. The oligonucleotide of any one of Embodiments 1-1576, wherein the nucleobase of N₋₁ is b009U.
1594. The oligonucleotide of any one of Embodiments 1-1576, wherein the nucleobase of N₋₁ is b011U.
1595. The oligonucleotide of any one of Embodiments 1-1576, wherein the nucleobase of N₋₁ is b012U.
1596. The oligonucleotide of any one of Embodiments 1-1576, wherein the nucleobase of N₋₁ is b013U.
1597. The oligonucleotide of any one of Embodiments 1-1576, wherein the nucleobase of N₋₁ is b014U.
1598. The oligonucleotide of any one of Embodiments 1-1576, wherein the nucleobase of N₋₁ is b015U.
1599. The oligonucleotide of any one of Embodiments 1-1576, wherein the nucleobase of N₋₁ is b001A.
1600. The oligonucleotide of any one of Embodiments 1-1576, wherein the nucleobase of N₋₁ is b002A.
1601. The oligonucleotide of any one of Embodiments 1-1576, wherein the nucleobase of N₋₁ is b003A.
1602. The oligonucleotide of any one of Embodiments 1-1576, wherein the nucleobase of N₋₁ is b004A.
1603. The oligonucleotide of any one of Embodiments 1-1576, wherein the nucleobase of N₋₁ is b005A.
1604. The oligonucleotide of any one of Embodiments 1-1576, wherein the nucleobase of N₋₁ is b006A.
1605. The oligonucleotide of any one of Embodiments 1-1576, wherein the nucleobase of N₋₁ is b007A.
1606. The oligonucleotide of any one of Embodiments 1-1576, wherein the nucleobase of N₋₁ is b001G.
1607. The oligonucleotide of any one of Embodiments 1-1576, wherein the nucleobase of N₋₁ is b002G.
1608. The oligonucleotide of any one of Embodiments 1-1576, wherein the nucleobase of N₋₁ is b001C.
1609. The oligonucleotide of any one of Embodiments 1-1576, wherein the nucleobase of N₋₁ is b002C.
1610. The oligonucleotide of any one of Embodiments 1-1576, wherein the nucleobase of N₋₁ is b003C.
1611. The oligonucleotide of any one of Embodiments 1-1576, wherein the nucleobase of N₁ is b004C.
1612. The oligonucleotide of any one of Embodiments 1-1576, wherein the nucleobase of N₋₁ is b005C.
1613. The oligonucleotide of any one of Embodiments 1-1576, wherein the nucleobase of N₋₁ is b006C.
1614. The oligonucleotide of any one of Embodiments 1-1576, wherein the nucleobase of N₋₁ is b007C.
1615. The oligonucleotide of any one of Embodiments 1-1576, wherein the nucleobase of N₋₁ is b008C.
1616. The oligonucleotide of any one of Embodiments 1-1576, wherein the nucleobase of N₋₁ is b009C.
1617. The oligonucleotide of any one of Embodiments 1-1576, wherein the nucleobase of N₋₁ is b002I.
1618. The oligonucleotide of any one of Embodiments 1-1576, wherein the nucleobase of N₋₁ is b003T.
1619. The oligonucleotide of any one of Embodiments 1-1576, wherein the nucleobase of N₋₁ is b004I.
1620. The oligonucleotide of any one of Embodiments 1-1576, wherein the nucleobase of N₋₁ is b014I.
1621. The oligonucleotide of any one of Embodiments 1-1576, wherein the nucleobase of N₋₁ is [3nT].
1622. The oligonucleotide of any one of Embodiments 1-1576, wherein the nucleobase of N₋₁ is [3ne5U].
1623. The oligonucleotide of any one of Embodiments 1-1576, wherein the nucleobase of N₋₁ is [3nfl5U].
1624. The oligonucleotide of any one of Embodiments 1-1576, wherein the nucleobase of N₋₁ is [3npry5U].
1625. The oligonucleotide of any one of Embodiments 1-1576, wherein the nucleobase of N₋₁ is [3ncn5U].
1626. The oligonucleotide of any one of Embodiments 1-1576, wherein the nucleobase of N₋₁ is [napth6o8A].
1627. The oligonucleotide of any one of Embodiments 1-1576, wherein the nucleobase of N₋₁ is [ipr6o8A].
1628. The oligonucleotide of any one of Embodiments 1-1576, wherein the nucleobase of N₋₁ is [c7In].
1629. The oligonucleotide of any one of Embodiments 1-1576, wherein the nucleobase of N₋₁ is [c39z48In].
1630. The oligonucleotide of any one of Embodiments 1-1576, wherein the nucleobase of N₋₁ is [z2c3In].
1631. The oligonucleotide of any one of Embodiments 1-1576, wherein the nucleobase of N₋₁ is [z5C].
1632. The oligonucleotide of any one of Embodiments 1-1576, wherein the nucleobase of N₋₁ is selected from Table BA-1.
1633. The oligonucleotide of any one of Embodiments 1-1576, wherein the nucleobase of N₋₁ is zndp.
1634. The oligonucleotide of any one of Embodiments 1-1576, wherein N₋₁ is dC.
1635. The oligonucleotide of any one of Embodiments 1-1576, wherein N₋₁ is fU.
1636. The oligonucleotide of any one of Embodiments 1-1576, wherein N₋₁ is dU.
1637. The oligonucleotide of any one of Embodiments 1-1576, wherein N₋₁ is fA.
1638. The oligonucleotide of any one of Embodiments 1-1576, wherein N₋₁ is dA.
1639. The oligonucleotide of any one of Embodiments 1-1576, wherein N₋₁ is fT.
1640. The oligonucleotide of any one of Embodiments 1-1576, wherein N₋₁ is dT.
1641. The oligonucleotide of any one of Embodiments 1-1576, wherein N₋₁ is fC.
1642. The oligonucleotide of any one of Embodiments 1-1576, wherein N₋₁ is fG.
1643. The oligonucleotide of any one of Embodiments 1-1576, wherein N₋₁ is dG.
1644. The oligonucleotide of any one of Embodiments 1-1576, wherein N₋₁ is dI.
1645. The oligonucleotide of any one of Embodiments 1-1576, wherein N₋₁ is fI.
1646. The oligonucleotide of any one of Embodiments 1-1576, wherein N₋₁ is aC.
1647. The oligonucleotide of any one of Embodiments 1-1576, wherein N₋₁ is m5dC.
1648. The oligonucleotide of any one of Embodiments 1-1576, wherein N₋₁ is 5MRmD5dC.
1649. The oligonucleotide of any one of Embodiments 1-1576, wherein N₋₁ is 5MSn5dC.
1650. The oligonucleotide of any one of Embodiments 1-1576, wherein N₋₁ is b001G.
1651. The oligonucleotide of any one of Embodiments 1-1576, wherein N₋₁ is b002G.
1652. The oligonucleotide of any one of Embodiments 1-1576, wherein N₋₁ is b001C.
1653. The oligonucleotide of any one of Embodiments 1-1576, wherein N₋₁ is b002C.
1654. The oligonucleotide of any one of Embodiments 1-1576, wherein N₋₁ is b003C.
1655. The oligonucleotide of any one of Embodiments 1-1576, wherein N₋₁ is b003mC.
1656. The oligonucleotide of any one of Embodiments 1-1576, wherein N₋₁ is b004C.
1657. The oligonucleotide of any one of Embodiments 1-1576, wherein N₋₁ is b005C.
1658. The oligonucleotide of any one of Embodiments 1-1576, wherein N₋₁ is b006C.
1659. The oligonucleotide of any one of Embodiments 1-1576, wherein N₋₁ is b007C.
1660. The oligonucleotide of any one of Embodiments 1-1576, wherein N₋₁ is b008C.
1661. The oligonucleotide of any one of Embodiments 1-1576, wherein N₋₁ is b009C.
1662. The oligonucleotide of any one of Embodiments 1-1576, wherein N₋₁ is b001A.
1663. The oligonucleotide of any one of Embodiments 1-1576, wherein N₋₁ is b002A.
1664. The oligonucleotide of any one of Embodiments 1-1576, wherein N₋₁ is b003A.
1665. The oligonucleotide of any one of Embodiments 1-1576, wherein N₋₁ is b004A.
1666. The oligonucleotide of any one of Embodiments 1-1576, wherein N₋₁ is b005A.
1667. The oligonucleotide of any one of Embodiments 1-1576, wherein N₋₁ is b006A.
1668. The oligonucleotide of any one of Embodiments 1-1576, wherein N₋₁ is b007A.
1669. The oligonucleotide of any one of Embodiments 1-1576, wherein N₋₁ is b001U.
1670. The oligonucleotide of any one of Embodiments 1-1576, wherein N₋₁ is b002U.
1671. The oligonucleotide of any one of Embodiments 1-1576, wherein N₋₁ is b003U.
1672. The oligonucleotide of any one of Embodiments 1-1576, wherein N₋₁ is b004U.
1673. The oligonucleotide of any one of Embodiments 1-1576, wherein N₋₁ is b005U.
1674. The oligonucleotide of any one of Embodiments 1-1576, wherein N₋₁ is b006U.
1675. The oligonucleotide of any one of Embodiments 1-1576, wherein N₋₁ is b007U.
1676. The oligonucleotide of any one of Embodiments 1-1576, wherein N₋₁ is b008U.
1677. The oligonucleotide of any one of Embodiments 1-1576, wherein N₋₁ is b009U.
1678. The oligonucleotide of any one of Embodiments 1-1576, wherein N₋₁ is b010U.
1679. The oligonucleotide of any one of Embodiments 1-1576, wherein N₋₁ is b011U.
1680. The oligonucleotide of any one of Embodiments 1-1576, wherein N₋₁ is b012U.
1681. The oligonucleotide of any one of Embodiments 1-1576, wherein N₋₁ is b013U.
1682. The oligonucleotide of any one of Embodiments 1-1576, wherein N₋₁ is b014U.
1683. The oligonucleotide of any one of Embodiments 1-1576, wherein N₋₁ is b015U.
1684. The oligonucleotide of any one of Embodiments 1-1576, wherein N₋₁ is b0021.
1685. The oligonucleotide of any one of Embodiments 1-1576, wherein N₋₁ is b0031.
1686. The oligonucleotide of any one of Embodiments 1-1576, wherein N₋₁ is b0041.
1687. The oligonucleotide of any one of Embodiments 1-1576, wherein N₋₁ is b0141.
1688. The oligonucleotide of any one of Embodiments 1-1576, wherein N₋₁ is [3nT].
1689. The oligonucleotide of any one of Embodiments 1-1576, wherein N₋₁ is [3ne5U].
1690. The oligonucleotide of any one of Embodiments 1-1576, wherein N₋₁ is [3nfl5U].
1691. The oligonucleotide of any one of Embodiments 1-1576, wherein N₋₁ is [3npry5U].
1692. The oligonucleotide of any one of Embodiments 1-1576, wherein N₋₁ is [3ncn5U].
1693. The oligonucleotide of any one of Embodiments 1-1576, wherein N₋₁ is [napth608A].
1694. The oligonucleotide of any one of Embodiments 1-1576, wherein N₋₁ is [ipr6o8A].
1695. The oligonucleotide of any one of Embodiments 1-1576, wherein N₋₁ is [c7In].
1696. The oligonucleotide of any one of Embodiments 1-1576, wherein N₋₁ is [c39z48In].
1697. The oligonucleotide of any one of Embodiments 1-1576, wherein N₋₁ is [z2c3In].
1698. The oligonucleotide of any one of Embodiments 1-1576, wherein N₋₁ is [z5C1.
1699. The oligonucleotide of any one of Embodiments 1-1576, wherein N₋₁ is Asm01.
1700. The oligonucleotide of any one of Embodiments 1-1576, wherein N₋₁ is Gsm01.
1701. The oligonucleotide of any one of Embodiments 1-1576, wherein N₋₁ is 5MSfC.
1702. The oligonucleotide of any one of Embodiments 1-1576, wherein N₋₁ is Usm04.
1703. The oligonucleotide of any one of Embodiments 1-1576, wherein N₋₁ is 5MRdT.
1704. The oligonucleotide of any one of Embodiments 1-1576, wherein N₋₁ is Csm04.
1705. The oligonucleotide of any one of Embodiments 1-1576, wherein N₋₁ is Csm11.
1706. The oligonucleotide of any one of Embodiments 1-1576, wherein N₋₁ is Gsm11.
1707. The oligonucleotide of any one of Embodiments 1-1576, wherein N₋₁ is Tsm11.
1708. The oligonucleotide of any one of Embodiments 1-1576, wherein N₋₁ is b009Csm11.
1709. The oligonucleotide of any one of Embodiments 1-1576, wherein N₋₁ is b009Csm12.
1710. The oligonucleotide of any one of Embodiments 1-1576, wherein N₋₁ is Gsm12.
1711. The oligonucleotide of any one of Embodiments 1-1576, wherein N₋₁ is Tsm12.
1712. The oligonucleotide of any one of Embodiments 1-1576, wherein N₋₁ is Csm12.
1713. The oligonucleotide of any one of Embodiments 1-1576, wherein N₋₁ is rCsm13.
1714. The oligonucleotide of any one of Embodiments 1-1576, wherein N₋₁ is rCsm14.
1715. The oligonucleotide of any one of Embodiments 1-1576, wherein N₋₁ is Csm15.
1716. The oligonucleotide of any one of Embodiments 1-1576, wherein N₋₁ is b001Asm15.
1717. The oligonucleotide of any one of Embodiments 1-1576, wherein N₋₁ is b008Usm15.
1718. The oligonucleotide of any one of Embodiments 1-1576, wherein N₋₁ is Csm16.
1719. The oligonucleotide of any one of Embodiments 1-1576, wherein N₋₁ is Csm17.
1720. The oligonucleotide of any one of Embodiments 1-1576, wherein N₋₁ is abasic.
1721. The oligonucleotide of any one of Embodiments 1-1576, wherein N₋₁ is L010.
1722. The oligonucleotide of any one of Embodiments 1-1576, wherein N₋₁ is L034.
1723. The oligonucleotide of any one of Embodiments 1-1576, wherein N₋₁ is Csm15.
1724. The oligonucleotide of any one of Embodiments 1-1576, wherein N₋₁ is Tsm18.
1725. The oligonucleotide of any one of Embodiments 1-1576, wherein N₋₁ is Csm19.
1726. The oligonucleotide of any one of Embodiments 1-1576, wherein N₋₁ is b008Usm19.
1727. The oligonucleotide of any one of Embodiments 1-1576, wherein N₋₁ is b001rA.
1728. The oligonucleotide of any one of the preceding Embodiments, wherein the internucleotidic linkage between N₀ and N₁ is a phosphorothioate internucleotidic linkage.
1729. The oligonucleotide of any one of the preceding Embodiments, wherein the internucleotidic linkage between N₀ and N₁ is a Sp phosphorothioate internucleotidic linkage.
1730. The oligonucleotide of any one of the preceding Embodiments, wherein the internucleotidic linkage between N₀ and N₋₁ is a phosphorothioate internucleotidic linkage.
1731. The oligonucleotide of any one of the preceding Embodiments, wherein the internucleotidic linkage between N₀ and N₋₁ is a Sp phosphorothioate internucleotidic linkage.
1732. The oligonucleotide of any one of the preceding Embodiments, wherein sugar of N₂ is a modified sugar.
1733. The oligonucleotide of any one of the preceding Embodiments, wherein sugar of N₂ is a 2′-F modified sugar.
1734. The oligonucleotide of any one of the preceding Embodiments, wherein the internucleotidic linkage between N₁ and N₂ is a phosphorothioate internucleotidic linkage.
1735. The oligonucleotide of any one of the preceding Embodiments, wherein the internucleotidic linkage between N₁ and N₂ is a Sp phosphorothioate internucleotidic linkage.
1736. The oligonucleotide of any one of the preceding Embodiments, wherein sugar of N₃ is a modified sugar.
1737. The oligonucleotide of any one of the preceding Embodiments, wherein sugar of N₃ is a 2′-OR modified sugar wherein R is optionally substituted C₁₋₆ aliphatic or a bicyclic sugar.
1738. The oligonucleotide of any one of the preceding Embodiments, wherein sugar of N₃ is a 2′-OR modified sugar wherein R is optionally substituted C₁₋₆ aliphatic.
1739. The oligonucleotide of any one of the preceding Embodiments, wherein sugar of N₃ is a 2′-OMe modified sugar.
1740. The oligonucleotide of Embodiment 1737, wherein sugar of N₃ is a 2′-MOE modified sugar.
1741. The oligonucleotide of any one of the preceding Embodiments, wherein the internucleotidic linkage between N₂ and N₃ is natural phosphate linkage.
1742. The oligonucleotide of any one of the preceding Embodiments, wherein sugar of N₄ is a modified sugar.
1743. The oligonucleotide of any one of the preceding Embodiments, wherein sugar of N₄ is a 2′-OR modified sugar wherein R is optionally substituted C₁₋₆ aliphatic or a bicyclic sugar.
1744. The oligonucleotide of any one of the preceding Embodiments, wherein sugar of N₄ is a 2′-OR modified sugar wherein R is optionally substituted C₁₋₆ aliphatic.
1745. The oligonucleotide of any one of the preceding Embodiments, wherein sugar of N₄ is a 2′-OMe modified sugar.
1746. The oligonucleotide of Embodiment 1743, wherein sugar of N₄ is a 2′-MOE modified sugar.
1747. The oligonucleotide of any one of the preceding Embodiments, wherein the internucleotidic linkage between N₃ and N₄ is a natural phosphate linkage.
1748. The oligonucleotide of any one of the preceding Embodiments, wherein sugar of N₅ is a modified sugar.
1749. The oligonucleotide of any one of the preceding Embodiments, wherein sugar of N₅ is a 2′-F modified sugar.
1750. The oligonucleotide of any one of the preceding Embodiments, wherein the internucleotidic linkage between N₄ and N₅ is a non-negatively charged internucleotidic linkage.
1751. The oligonucleotide of any one of the preceding Embodiments, wherein the internucleotidic linkage between N₄ and N₅ is a phosphoryl guanidine internucleotidic linkage.
1752. The oligonucleotide of any one of the preceding Embodiments, wherein the internucleotidic linkage between N₄ and N₅ is n001.
1753. The oligonucleotide of any one of the preceding Embodiments, wherein the internucleotidic linkage between N₄ and N₅ is Rp n001.
1754. The oligonucleotide of any one of the preceding Embodiments, wherein sugar of N₆ is a modified sugar.
1755. The oligonucleotide of any one of the preceding Embodiments, wherein sugar of N₆ is a 2′-F modified sugar.
1756. The oligonucleotide of any one of the preceding Embodiments, wherein the internucleotidic linkage between N₅ and N₆ is a phosphorothioate internucleotidic linkage internucleotidic linkage.
1757. The oligonucleotide of any one of the preceding Embodiments, wherein the internucleotidic linkage between N₅ and N₆ is a Sp phosphorothioate internucleotidic linkage internucleotidic linkage.
1758. The oligonucleotide of any one of the preceding Embodiments, wherein sugar of N₋₂ is a modified sugar.
1759. The oligonucleotide of any one of the preceding Embodiments, wherein sugar of N₋₂ is a 2′-OR modified sugar wherein R is optionally substituted C₁₋₆ aliphatic or a bicyclic sugar.
1760. The oligonucleotide of any one of the preceding Embodiments, wherein sugar of N₋₂ is a 2′-OR modified sugar wherein R is optionally substituted C₁₋₆ aliphatic.
1761. The oligonucleotide of any one of the preceding Embodiments, wherein sugar of N₋₂ is a 2′-OMe modified sugar.
1762. The oligonucleotide of Embodiment 1759, wherein sugar of N₋₂ is a 2′-MOE modified sugar.
1763. The oligonucleotide of any one of the preceding Embodiments, wherein the internucleotidic linkage between N₋₁ and N₋₂ is a non-negatively charged internucleotidic linkage.
1764. The oligonucleotide of any one of the preceding Embodiments, wherein the internucleotidic linkage between N₋₁ and N₋₂ is a phosphoryl guanidine internucleotidic linkage.
1765. The oligonucleotide of any one of the preceding Embodiments, wherein the internucleotidic linkage between N₋₁ and N₋₂ is n004, n008, n025, n026.
1766. The oligonucleotide of any one of the preceding Embodiments, wherein the internucleotidic linkage between N₋₁ and N₋₂ is Rp n004, n008, n025, n026.
1767. The oligonucleotide of any one of the preceding Embodiments, wherein the internucleotidic linkage between N₋₁ and N₋₂ is Sp n004, n008, n025, n026.
1768. The oligonucleotide of any one of the preceding Embodiments, wherein the internucleotidic linkage between N₋₁ and N₋₂ is n001.
1769. The oligonucleotide of any one of the preceding Embodiments, wherein the internucleotidic linkage between N₋₁ and N₋₂ is Rp n001.
1770. The oligonucleotide of Embodiment 1768, wherein the internucleotidic linkage between N₋₁ and N₋₂ is Sp n001.
1771. The oligonucleotide of any one of the preceding Embodiments, wherein sugar of N₋₃ is a modified sugar.
1772. The oligonucleotide of any one of the preceding Embodiments, wherein sugar of N₋₃ is a 2′-F modified sugar.
1773. The oligonucleotide of any one of the preceding Embodiments, wherein the internucleotidic linkage between N₋₂ and N₋₃ is a natural phosphate linkage.
1774. The oligonucleotide of any one of the preceding Embodiments, wherein sugar of N₋₄ is a modified sugar.
1775. The oligonucleotide of any one of the preceding Embodiments, wherein sugar of N₋₄ is a 2′-OR modified sugar wherein R is optionally substituted C₁₋₆ aliphatic or a bicyclic sugar.
1776. The oligonucleotide of any one of the preceding Embodiments, wherein sugar of N₋₄ is a 2′-OR modified sugar wherein R is optionally substituted C₁₋₆ aliphatic.
1777. The oligonucleotide of any one of the preceding Embodiments, wherein sugar of N₋₄ is a 2′-OMe modified sugar.
1778. The oligonucleotide of Embodiment 1775, wherein sugar of N₋₄ is a 2′-MOE modified sugar.
1779. The oligonucleotide of any one of the preceding Embodiments, wherein the linkage between N₋₃ and N₋₄ is a phosphorothioate internucleotidic linkage.
1780. The oligonucleotide of any one of the preceding Embodiments, wherein the linkage between N₋₃ and N₋₄ is a Sp phosphorothioate internucleotidic linkage.
1781. The oligonucleotide of any one of the preceding Embodiments, wherein sugar of N₋₅ is a modified sugar.
1782. The oligonucleotide of any one of the preceding Embodiments, wherein sugar of N₋₅ is a 2′-OR modified sugar wherein R is optionally substituted C₁₋₆ aliphatic or a bicyclic sugar.
1783. The oligonucleotide of any one of the preceding Embodiments, wherein sugar of N₋₅ is a 2′-OR modified sugar wherein R is optionally substituted C₁₋₆ aliphatic.
1784. The oligonucleotide of any one of the preceding Embodiments, wherein sugar of N₋₅ is a 2′-OMe modified sugar.
1785. The oligonucleotide of Embodiment 1782, wherein sugar of N₋₅ is a 2′-MOE modified sugar.
1786. The oligonucleotide of any one of the preceding Embodiments, wherein the linkage between N₋₄ and N₋₅ is a phosphorothioate internucleotidic linkage.
1787. The oligonucleotide of any one of the preceding Embodiments, wherein the linkage between N₋₄ and N₋₅ is a Sp phosphorothioate internucleotidic linkage.
1788. The oligonucleotide of any one of the preceding Embodiments, wherein sugar of N₋₆ is a modified sugar.
1789. The oligonucleotide of any one of the preceding Embodiments, wherein sugar of N₋₆ is a 2′-OR modified sugar wherein R is optionally substituted C₁₋₆ aliphatic or a bicyclic sugar.
1790. The oligonucleotide of any one of the preceding Embodiments, wherein sugar of N₋₆ is a 2′-OR modified sugar wherein R is optionally substituted C₁₋₆ aliphatic.
1791. The oligonucleotide of any one of the preceding Embodiments, wherein sugar of N₋₆ is a 2′-OMe modified sugar.
1792. The oligonucleotide of Embodiment 1782, wherein sugar of N₋₆ is a 2′-MOE modified sugar.
1793. The oligonucleotide of any one of the preceding Embodiments, wherein the internucleotidic linkage between N₋₅ and N₋₆ is a non-negatively charged internucleotidic linkage.
1794. The oligonucleotide of any one of the preceding Embodiments, wherein the internucleotidic linkage between N₋₅ and N₋₆ is a phosphoryl guanidine internucleotidic linkage.
1795. The oligonucleotide of any one of the preceding Embodiments, wherein the internucleotidic linkage between N₋₅ and N₋₆ is n001.
1796. The oligonucleotide of any one of the preceding Embodiments, wherein the internucleotidic linkage between N₋₅ and N₋₆ is Rp n001.
1797. The oligonucleotide of any one of the preceding Embodiments, wherein about 20%-80%, 30-70%, 30%-60%, 30%-50%, 40%-60%, 20%, 30%, 40%, 50%, 60%, 70% or 80% of its sugars are each independently a 2′-F modified sugar.
1798. The oligonucleotide of any one of the preceding Embodiments, wherein about 30%-60% of its sugars are each independently a 2′-F modified sugar.
1799. The oligonucleotide of any one of the preceding Embodiments, wherein about 20%-80%, 30-70%, 30%-60%, 30%-50%, 40%-60%, 20%, 30%, 40%, 50%, 60%, 70% or 80% of its sugars are each independently a 2′-OR modified sugar wherein R is optionally substituted C₁₋₆ aliphatic.
1800. The oligonucleotide of any one of the preceding Embodiments, wherein about 30%-60% of its sugars are each independently a 2′-OR modified sugar wherein R is optionally substituted C₁₋₆ aliphatic.
1801. The oligonucleotide of any one of the preceding Embodiments, wherein about 30%-60% of its sugars are each independently a 2′-OMe or 2′-MOE modified sugar.
1802. The oligonucleotide of any one of the preceding Embodiments, wherein about 20%-80%, 30-70%, 30%-60%, 30%-50%, 40%-60%, 20%0, 30%, 40%, 50% 60%, 70% or 80% of its sugars in a first domain are each independently a 2′-OR modified sugar wherein R is optionally substituted C₁₋₆ aliphatic.
1803. The oligonucleotide of any one of the preceding Embodiments, wherein about 30%-60% of its sugars in a first domain are each independently a 2′-OR modified sugar wherein R is optionally substituted C₁₋₆ aliphatic.
1804. The oligonucleotide of any one of the preceding Embodiments, wherein about 30%-60% of its sugars in a first domain are each independently a 2′-OMe or 2′-MOE modified sugar.
1805. The oligonucleotide of any one of the preceding Embodiments, wherein the 3′-end nucleoside of a first domain is N₂.
1806. The oligonucleotide of any one of the preceding Embodiments, wherein the 5′-end nucleoside of a first domain is the 5′-end nucleoside of the oligonucleotide.
1807. The oligonucleotide of any one of the preceding Embodiments, wherein about or at least about 20%, 30%, 40%, 50%, 60%, 70%, 80% or 90% 2′-OR modified sugars wherein R is optionally substituted C₁₋₆ aliphatic in an oligonucleotide or a portion thereof, e.g., a first domain, a second domain, etc., are independently bonded to a natural phosphate linkage.
1808. The oligonucleotide of any one of the preceding Embodiments, wherein each of one or more sugars of N₋₂, N₋₃, N₋₄, N₋₅, and N₋₆ is independently a 2′-OR modified sugar wherein R is optionally substituted C₁₋₆ aliphatic or a bicyclic sugar.
1809. The oligonucleotide of any one of the preceding Embodiments, wherein each of one or more sugars of N₋₂, N₋₃, N₋₄, N₋₅, and N₋₆ is independently a 2′-OR modified sugar wherein R is optionally substituted C₁₋₆ aliphatic.
1810. The oligonucleotide of any one of the preceding Embodiments, wherein each of one or more sugars of N₋₂, N₋₃, N₋₄, N₋₅, and N₋₆ is independently a 2′-OMe modified sugar.
1811. The oligonucleotide of any one of the preceding Embodiments, wherein each of one or more sugars of N₋₂, N₋₃, N₋₄, N₋₅, and N₋₆ is independently a 2′-MOE modified sugar.
1812. The oligonucleotide of any one of the preceding Embodiments, wherein each of one or more sugars of N₂, N₃, N₄, N₅, N₆, N₇, and N₈ is independently a 2′-OR modified sugar wherein R is optionally substituted C₁₋₆ aliphatic or a bicyclic sugar.
1813. The oligonucleotide of any one of the preceding Embodiments, wherein each of one or more sugars of N₂, N₃, N₄, N₅, N₆, N₇, and N₈ is independently a 2′-OR modified sugar wherein R is optionally substituted C₁₋₆ aliphatic.
1814. The oligonucleotide of any one of the preceding Embodiments, wherein each of one or more sugars of N₂, N₃, N4, N₅, N₆, N₇, and N₈ is independently a 2′-OMe modified sugar.
1815. The oligonucleotide of any one of the preceding Embodiments, wherein each of one or more sugars of N₂, N₃, N₄, N₅, N₆, N₇, and N₈ is independently a 2′-MOE modified sugar.
1816. The oligonucleotide of any one of the preceding Embodiments, wherein about or at least about 50% 2′-OR modified sugars wherein R is optionally substituted C₁ aliphatic in an oligonucleotide or a portion thereof, e.g., a first domain, a second domain, etc., are independently bonded to a natural phosphate linkage.
1817. The oligonucleotide of any one of the preceding Embodiments, wherein at least 60%, 70%, 80% or 90% or all natural phosphate linkages each independently bond to at least one modified sugar which is 2′-OR modified sugar wherein R is optionally substituted C₁₋₆ aliphatic or a bicyclic sugar.
1818. The oligonucleotide of any one of the preceding Embodiments, wherein about 5%-90%, about 10-80%, about 10-75%, about 10-70%, 10%-60%, 10-50%, 10-40%, 10-30%, 15-40%, 20-30%, 25-30%, or about or at least about 5%, 10%, 15%, 20%, 25%, 30%, 35%, 40%, 45%, or 50%, of all internucleotidic linkages in an oligonucleotide are independently a natural phosphate linkage.
1819. The oligonucleotide of any one of the preceding Embodiments, wherein about 5%-90%, about 10-80%, about 10-75%, about 10-70%, 10%-60%, 10-50%, 10-40%, 10-30%, 15-40%, 20-30%, 25-30%, or about or at least about 5%, 10%, 15%, 20%, 25%, 30%, 35%, 40%, 45%, or 50%, of all internucleotidic linkages in a first domain are independently a natural phosphate linkage.
1820. The oligonucleotide of any one of the preceding Embodiments, wherein one or more internucleotidic linkages at one or more of positions +3 (between N₊₄N+₃), +4, +6, +8, +9, +12, +14, +15, +17, and +18 are independently a natural phosphate linkage.
1821. The oligonucleotide of any one of the preceding Embodiments, wherein about 5%-90%, about 10-80%, about 10-75%, about 10-70%, 10%-60%, 10-50%, 10-40%, 10-30%, 15-40%, 20-30%, 25-30%, 30%-70%, 40-70%, 40%-65%, 40%-60%, or about or at least about 5%, 10%, 15%, 20%, 25%, 30%, 35%, 40%, 45%, 50%, 55%, 60%, or 65% of all internucleotidic linkages in an oligonucleotide are independently a phosphorothioate internucleotidic linkage.
1822. The oligonucleotide of any one of the preceding Embodiments, wherein about 5%-90%, about 10-80%, about 10-75%, about 10-70%, 10%-60%, 10-50%, 10-40%, 10-30%, 15-40%, 20-30%, 25-30%, 30%-70%, 40-70%, 40%-65%, 40%-⁶⁰%, or about or at least about 5%, 10%, 15%, 20%, 25%, 30%, 35%, 40%, 45%, 50%, 55%, 60%, or 65% of all internucleotidic linkages in a first domain are independently a phosphorothioate internucleotidic linkage.
1823. The oligonucleotide of any one of the preceding Embodiments, wherein one or more internucleotidic linkages at one or more of positions +1 (between N₊₁N₀), +2, +5, +6, +7, +8, +11, +14, +15, +16, +17, +19, +20, +21, and +22 are independently a phosphorothioate internucleotidic linkage.
1824. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%. 15%-30%, 15-25%, 15-20%, or about or at least about 5%, 10%, 15%, 20%, 25%, 30%, 35%, 40%, 45%, or 50%, of all internucleotidic linkages in an oligonucleotide are independently a non-negatively charged internucleotidic linkage.
1825. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%, 15%-30%, 15-25%, 15-20%, or about or at least about 5%, 10%, 15%, 20%, 25%, 30%, 35%, 40%, 45%, or 50%, of all internucleotidic linkages in a first domain are independently a non-negatively charged internucleotidic linkage.
1826. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%. 15%-30%, 15-25%, 15-20%, or about or at least about 5%, 10%, 15%, 20%, 25%, 30%, 35%, 40%, 45%, or 50%, of all internucleotidic linkages in an oligonucleotide are independently a phosphoryl guanidine internucleotidic linkage.
1827. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%. 15%-30%, 15-25%, 15-20%, or about or at least about 5%, 10%, 15%, 20%, 25%, 30%, 35%, 40%, 45%, or 50%, of all internucleotidic linkages in a first domain are independently a phosphoryl guanidine internucleotidic linkage.
1828. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%. 15%-30%, 15-25%, 15-20%, or about or at least about 5%, 10%, 15%, 20%, 25%, 30%, 35%, 40%, 45%, or 50%, of all internucleotidic linkages in an oligonucleotide are independently n001.
1829. The oligonucleotide of any one of the preceding Embodiments, wherein about 5%-90%, about 10-80%, about 10-75%, about 10-70%, 10%-60%, 10-50%, 10-40%, 10-30%, 10%-20%, 10-15%, 15-40%, 15%-35%. 15%-30%, 15-25%, 15-20%, or about or at least about 5%, 10%, 15%, 20%, 25%, 30%, 35%, 40%, 45%, or 50%, of all internucleotidic linkages in a first domain are independently n001.
1830. The oligonucleotide of any one of the preceding Embodiments, wherein one or more or all of positions +5 (between N₊₅N₊₄), +10, +13 or +23 are independently a non-negatively charged internucleotidic linkage.
1831. The oligonucleotide of any one of the preceding Embodiments, wherein one or more or all of positions +5 (between N₊₅N₊₄), +10, +13 or +23 are independently a phosphoryl guanidine internucleotidic linkage.
1832. The oligonucleotide of any one of the preceding Embodiments, wherein one or more or all of positions +5 (between N₊₅N₊₄), +10, +13 or +23 are independently n001.
1833. The oligonucleotide of any one of the preceding Embodiments, wherein sugar of N₄ is a 2′-OR modified sugar wherein R is optionally substituted C₁₋₆ aliphatic.
1834. The oligonucleotide of any one of the preceding Embodiments, wherein the internucleotidic linkage between N₃ and N₄ is natural phosphate linkage.
1835. The oligonucleotide of any one of the preceding Embodiments, wherein there are 5 or more nucleosides at the 3′ side of N₀.
1836. The oligonucleotide of any one of the preceding Embodiments, wherein there are 6 or more nucleosides at the 3′ side of N₀.
1837. The oligonucleotide of any one of the preceding Embodiments, wherein there are 7 or more nucleosides at the 3′ side of N₀.
1838. The oligonucleotide of any one of the preceding Embodiments, wherein there are 8 or more nucleosides at the 3′ side of N₀.
1839. The oligonucleotide of any one of Embodiments 1-1834, wherein there are 3 nucleosides at the 3′ side of N₀.
1840. The oligonucleotide of any one of Embodiments 1-1834, wherein there are 4 nucleosides at the 3′ side of N₀.
1841. The oligonucleotide of any one of Embodiments 1-1834, wherein there are 5 nucleosides at the 3′ side of N₀.
1842. The oligonucleotide of any one of Embodiments 1-1834, wherein there are 6 nucleosides at the 3′ side of N₀.
1843. The oligonucleotide of any one of Embodiments 1-1834, wherein there are 7 nucleosides at the 3′ side of N₀.
1844. The oligonucleotide of any one of Embodiments 1-1834, wherein there are 8 nucleosides at the 3′ side of N₀.
1845. The oligonucleotide of any one of Embodiments 1-1834, wherein there are 9 nucleosides at the 3′ side of N₀.
1846. The oligonucleotide of any one of Embodiments 1-1834, wherein there are 10 nucleosides at the 3′ side of N₀.
1847. The oligonucleotide of any one of the preceding Embodiments, wherein there are 5 or more (e.g., 5, 6, 7, 8, 9, 10, 11, 12, 13, 14, 15, 16, 17, 18, 19, 20, 21, 22, 23, 24, 25, 26, 27, 28, 29 or 30 or more) nucleosides at the 5′ side of N₀.
1848. The oligonucleotide of any one of the preceding Embodiments, wherein there are 8 or more nucleosides at the 5′ side of N₀.
1849. The oligonucleotide of any one of the preceding Embodiments, wherein there are 10 or more nucleosides at the 5′ side of N₀.
1850. The oligonucleotide of any one of the preceding Embodiments, wherein there are 15 or more nucleosides at the 5′ side of N₀.
1851. The oligonucleotide of any one of the preceding Embodiments, wherein there are 16 or more nucleosides at the 5′ side of N₀.
1852. The oligonucleotide of any one of the preceding Embodiments, wherein there are 17 or more nucleosides at the 5′ side of N₀.
1853. The oligonucleotide of any one of the preceding Embodiments, wherein there are 18 or more nucleosides at the 5′ side of N₀.
1854. The oligonucleotide of any one of the preceding Embodiments, wherein there are 19 or more nucleosides at the 5′ side of N₀.
1855. The oligonucleotide of any one of the preceding Embodiments, wherein there are 20 or more nucleosides at the 5′ side of N₀.
1856. The oligonucleotide of any one of the preceding Embodiments, wherein there are 21 or more nucleosides at the 5′ side of N₀.
1857. The oligonucleotide of any one of the preceding Embodiments, wherein there are 22 or more nucleosides at the 5′ side of N₀.
1858. The oligonucleotide of any one of the preceding Embodiments, wherein there are 23 or more nucleosides at the 5′ side of N₀.
1859. The oligonucleotide of any one of the preceding Embodiments, wherein there are 24 or more nucleosides at the 5′ side of N₀.
1860. The oligonucleotide of any one of the preceding Embodiments, wherein there are 25 or more nucleosides at the 5′ side of N₀.
1861. The oligonucleotide of any one of the preceding Embodiments, wherein there are 26 or more nucleosides at the 5′ side of N₀.
1862. The oligonucleotide of any one of Embodiments 1-1847, wherein there are 20 nucleosides at the 5′ side of N₀.
1863. The oligonucleotide of any one of Embodiments 1-1847, wherein there are 21 nucleosides at the 5′ side of N₀.
1864. The oligonucleotide of any one of Embodiments 1-1847, wherein there are 22 nucleosides at the 5′ side of N₀.
1865. The oligonucleotide of any one of Embodiments 1-1847, wherein there are 23 nucleosides at the 5′ side of N₀.
1866. The oligonucleotide of any one of Embodiments 1-1847, wherein there are 24 nucleosides at the 5′ side of N₀.
1867. The oligonucleotide of any one of Embodiments 1-1847, wherein there are 25 nucleosides at the 5′ side of N₀.
1868. The oligonucleotide of any one of Embodiments 1-1847, wherein there are 26 nucleosides at the 5′ side of N₀.
1869. The oligonucleotide of any one of Embodiments 1-1847, wherein there are 27 nucleosides at the 5′ side of N₀.
1870. The oligonucleotide of any one of Embodiments 1-1847, wherein there are 28 nucleosides at the 5′ side of N₀.
1871. The oligonucleotide of any one of Embodiments 1-1847, wherein there are 29 nucleosides at the 5′ side of N₀.
1872. The oligonucleotide of any one of Embodiments 1-1847, wherein there are 30 nucleosides at the 5′ side of N₀.
1873. The oligonucleotide of any one of the preceding Embodiments, wherein the first 1, 2, 3, 4, or 5 sugars at the 5′-end of the oligonucleotide are each independently sugars that can increase stability.
1874. The oligonucleotide of any one of the preceding Embodiments, wherein the first 3 sugars at the 5-end of the oligonucleotide are each independently sugars that can increase stability.
1875. The oligonucleotide of any one of the preceding Embodiments, wherein the first 4 sugars at the 5′-end of the oligonucleotide are each independently sugars that can increase stability.
1876. The oligonucleotide of any one of the preceding Embodiments, wherein the first 5 sugars at the 5′-end of the oligonucleotide are each independently sugars that can increase stability.
1877. The oligonucleotide of any one of the preceding Embodiments, wherein the first 1, 2, 3, 4, or 5 sugars at the 5′-end of the oligonucleotide are each independently selected from a bicyclic sugar and a 2′-OR modified sugar, wherein R is optionally substituted C₁₋₆ aliphatic.
1878. The oligonucleotide of any one of the preceding Embodiments, wherein the first 3 sugars at the 5′-end of the oligonucleotide are each independently selected from a bicyclic sugar and a 2′-OR modified sugar, wherein R is optionally substituted C₁₋₆ aliphatic.
1879. The oligonucleotide of any one of the preceding Embodiments, wherein the first 4 sugars at the 5-end of the oligonucleotide are each independently selected from a bicyclic sugar and a 2′-OR modified sugar, wherein R is optionally substituted C₁₋₆ aliphatic.
1880. The oligonucleotide of any one of the preceding Embodiments, wherein the first 5 sugars at the 5′-end of the oligonucleotide are each independently selected from a bicyclic sugar and a 2′-OR modified sugar, wherein R is optionally substituted C₁₋₆ aliphatic.
1881. The oligonucleotide of any one of the preceding Embodiments, wherein the first 3 sugars at the 5′-end of the oligonucleotide are each independently a 2′-OR modified sugar, wherein R is optionally substituted C₁₋₆ aliphatic.
1882. The oligonucleotide of any one of the preceding Embodiments, wherein the first 4 sugars at the 5′-end of the oligonucleotide are each independently a 2′-OR modified sugar, wherein R is optionally substituted C₁₋₆ aliphatic.
1883. The oligonucleotide of any one of the preceding Embodiments, wherein the first 5 sugars at the 5′-end of the oligonucleotide are each independently a 2′-OR modified sugar, wherein R is optionally substituted C₁₋₆ aliphatic.
1884. The oligonucleotide of any one of the preceding Embodiments, wherein the first 1, 2, 3, 4, or 5 sugars at the 5′-end of the oligonucleotide are each independently a 2′-OMe or 2′-MOE modified sugar.
1885. The oligonucleotide of any one of the preceding Embodiments, wherein the first 1, 2, 3, 4, or 5 sugars at the 5′-end of the oligonucleotide are each independently a 2′-OMe modified sugar.
1886. The oligonucleotide of any one of the preceding Embodiments, wherein the first 3 sugars at the 5′-end of the oligonucleotide are each independently a 2′-OMe modified sugar.
1887. The oligonucleotide of any one of the preceding Embodiments, wherein the first 4 sugars at the 5-end of the oligonucleotide are each independently a 2′-OMe modified sugar.
1888. The oligonucleotide of any one of the preceding Embodiments, wherein the first 5 sugars at the 5′-end of the oligonucleotide are each independently a 2′-OMe modified sugar.
1889. The oligonucleotide of any one of Embodiments 1-1884, wherein the first 1, 2, 3, 4, or 5 sugars at the 5′-end of the oligonucleotide are each independently a 2′-MOE modified sugar.
1890. The oligonucleotide of any one of Embodiments 1-1884, wherein the first 3 sugars at the 5′-end of the oligonucleotide are each independently a 2′-MOE modified sugar.
1891. The oligonucleotide of any one of Embodiments 1-1884, wherein the first 4 sugars at the 5′-end of the oligonucleotide are each independently a 2′-MOE modified sugar.
1892. The oligonucleotide of any one of Embodiments 1-1884, wherein the first 5 sugars at the 5′-end of the oligonucleotide are each independently a 2′-MOE modified sugar.
1893. The oligonucleotide of any one of the preceding Embodiments, wherein the last 1, 2, 3, 4, or 5 sugars at the 3′-end of the oligonucleotide are each independently sugars that can increase stability.
1894. The oligonucleotide of any one of the preceding Embodiments, wherein the last 3 sugars at the 3′-end of the oligonucleotide are each independently sugars that can increase stability.
1895. The oligonucleotide of any one of the preceding Embodiments, wherein the last 4 sugars at the 3′-end of the oligonucleotide are each independently sugars that can increase stability.
1896. The oligonucleotide of any one of the preceding Embodiments, wherein the last 5 sugars at the 3′-end of the oligonucleotide are each independently sugars that can increase stability.
1897. The oligonucleotide of any one of the preceding Embodiments, wherein the last 1, 2, 3, 4, or 5 sugars at the 3′-end of the oligonucleotide are each independently selected from a bicyclic sugar and a 2′-OR modified sugar, wherein R is optionally substituted C₁₋₆ aliphatic.
1898. The oligonucleotide of any one of the preceding Embodiments, wherein the last 3 sugars at the 3′-end of the oligonucleotide are each independently selected from a bicyclic sugar and a 2′-OR modified sugar, wherein R is optionally substituted C₁₋₆ aliphatic.
1899. The oligonucleotide of any one of the preceding Embodiments, wherein the last 4 sugars at the 3′-end of the oligonucleotide are each independently selected from a bicyclic sugar and a 2′-OR modified sugar, wherein R is optionally substituted C₁₋₆ aliphatic.
1900. The oligonucleotide of any one of the preceding Embodiments, wherein the last 5 sugars at the 3′-end of the oligonucleotide are each independently selected from a bicyclic sugar and a 2′-OR modified sugar, wherein R is optionally substituted C₁₋₆ aliphatic.
1901. The oligonucleotide of any one of the preceding Embodiments, wherein the last 3 sugars at the 3′-end of the oligonucleotide are each independently a 2′-OR modified sugar, wherein R is optionally substituted C₁₋₆ aliphatic.
1902. The oligonucleotide of any one of the preceding Embodiments, wherein the last 4 sugars at the 3′-end of the oligonucleotide are each independently a 2′-OR modified sugar, wherein R is optionally substituted C₁₋₆ aliphatic.
1903. The oligonucleotide of any one of the preceding Embodiments, wherein the last 5 sugars at the 3′-end of the oligonucleotide are each independently a 2′-OR modified sugar, wherein R is optionally substituted C₁₋₆ aliphatic.
1904. The oligonucleotide of any one of the preceding Embodiments, wherein the last 1, 2, 3, 4, or 5 sugars at the 3′-end of the oligonucleotide are each independently a 2′-OMe or 2′-MOE modified sugar.
1905. The oligonucleotide of any one of the preceding Embodiments, wherein the last 1, 2, 3, 4, or 5 sugars at the 3′-end of the oligonucleotide are each independently a 2′-OMe modified sugar.
1906. The oligonucleotide of any one of the preceding Embodiments, wherein the last 3 sugars at the 3′-end of the oligonucleotide are each independently a 2′-OMe modified sugar.
1907. The oligonucleotide of any one of the preceding Embodiments, wherein the last 4 sugars at the 3′-end of the oligonucleotide are each independently a 2′-OMe modified sugar.
1908. The oligonucleotide of any one of the preceding Embodiments, wherein the last 5 sugars at the 3′-end of the oligonucleotide are each independently a 2′-OMe modified sugar.
1909. The oligonucleotide of any one of Embodiments 1-1904, wherein the last 1, 2, 3, 4, or 5 sugars at the 3′-end of the oligonucleotide are each independently a 2′-MOE modified sugar.
1910. The oligonucleotide of any one of Embodiments 1-1904, wherein the last 3 sugars at the 3′-end of the oligonucleotide are each independently a 2′-MOE modified sugar.
1911. The oligonucleotide of any one of Embodiments 1-1904, wherein the last 4 sugars at the 3′-end of the oligonucleotide are each independently a 2′-MOE modified sugar.
1912. The oligonucleotide of any one of Embodiments 1-1904, wherein the last 5 sugars at the 3′-end of the oligonucleotide are each independently a 2′-MOE modified sugar.
1913. The oligonucleotide of any one of the preceding Embodiments, wherein the first internucleotidic linkage from the 5′-end of an oligonucleotide is a non-negatively charged internucleotidic linkage.
1914. The oligonucleotide of any one of the preceding Embodiments, wherein the first internucleotidic linkage from the 5′-end of an oligonucleotide is a neutral internucleotidic linkage.
1915. The oligonucleotide of any one of the preceding Embodiments, wherein the first internucleotidic linkage from the 5′-end of an oligonucleotide is phosphoryl guanidine internucleotidic linkage.
1916. The oligonucleotide of any one of the preceding Embodiments, wherein the first internucleotidic linkage from the 5′-end of an oligonucleotide is n004, n008, n025, n026.
1917. The oligonucleotide of any one of the preceding Embodiments, wherein the first internucleotidic linkage from the 5′-end of an oligonucleotide is n001.
1918. The oligonucleotide of any one of the preceding Embodiments, wherein the first internucleotidic linkage from the 5′-end is chirally controlled and is Rp.
1919. The oligonucleotide of any one of Embodiments 1-1917, wherein the first internucleotidic linkage from the 5′-end is chirally controlled and is Sp.
1920. The oligonucleotide of any one of the preceding Embodiments, wherein both internucleotidic linkages bonded to the 3^{rd} nucleosides from the 5′-end are each independently a phosphorothioate internucleotidic linkages.
1921. The oligonucleotide of any one of the preceding Embodiments, wherein both internucleotidic linkages bonded to the 4^{th} nucleosides from the 5′-end are each independently a phosphorothioate internucleotidic linkages.
1922. The oligonucleotide of any one of the preceding Embodiments, wherein both internucleotidic linkages bonded to the 5^{th} nucleosides from the 5′-end are each independently a phosphorothioate internucleotidic linkages.
1923. The oligonucleotide of any one of Embodiments 1920-1922, wherein each phosphorothioate internucleotidic linkage is chirally controlled.
1924. The oligonucleotide of Embodiment 1923, wherein each phosphorothioate internucleotidic linkage is Sp.
1925. The oligonucleotide of any one of the preceding Embodiments, wherein the first internucleotidic linkage from the 3′-end of an oligonucleotide is a non-negatively charged internucleotidic linkage.
1926. The oligonucleotide of any one of the preceding Embodiments, wherein the first internucleotidic linkage from the 3′-end of an oligonucleotide is a neutral internucleotidic linkage.
1927. The oligonucleotide of any one of the preceding Embodiments, wherein the first internucleotidic linkage from the 3′-end of an oligonucleotide is phosphoryl guanidine internucleotidic linkage.
1928. The oligonucleotide of any one of the preceding Embodiments, wherein the first internucleotidic linkage from the 3′-end of an oligonucleotide is n004, n008, n025, n026.
1929. The oligonucleotide of any one of the preceding Embodiments, wherein the first internucleotidic linkage from the 3′-end of an oligonucleotide is n001.
1930. The oligonucleotide of any one of the preceding Embodiments, wherein the first internucleotidic linkage from the 3′-end is chirally controlled and is Rp.
1931. The oligonucleotide of any one of Embodiments 1-1930, wherein the first internucleotidic linkage from the 3′-end is chirally controlled and is Sp.
1932. The oligonucleotide of any one of the preceding Embodiments, wherein the sugar of the nucleoside opposite to a target adenosine (position 0, such a nucleoside: No) is a natural DNA sugar.
1933. The oligonucleotide of any one of the preceding Embodiments, wherein the sugar of the nucleoside at position +1 (the nucleoside to the immediate 5′-side of No; i.e., N₊₁ of 5′- . . . N₊₁N₀ . . . -3′) is a natural DNA sugar.
1934. The oligonucleotide of any one of Embodiments 1-1932, wherein the sugar of the nucleoside at position +1 (the nucleoside to the immediate 5′-side of N₀; i.e., N₊₁ of 5′- . . . N₊₁N₀ . . . -3′) is a 2′-F modified sugar.
1935. The oligonucleotide of any one of the preceding Embodiments, wherein the sugar of the nucleoside at position +2 (N₊₂ of 5′- . . . N₋₂N₊₁N₀ . . . -3′) is a 2′-F modified sugar.
1936. The oligonucleotide of any one of the preceding Embodiments, wherein the sugar of the nucleoside at position −1 (N₋₁ of 5′- . . . N₊₂N₊₁N₀N₋₁ . . . -3′) is a natural DNA sugar.
1937. The oligonucleotide of any one of the preceding Embodiments, wherein the sugar of the nucleoside at position −2 (N₋₂ of 5′- . . . N₊₂N₊₁N₀N₋₁N₋₂ . . . -3′) is a sugar that can increase stability.
1938. The oligonucleotide of any one of the preceding Embodiments, wherein the sugar of the nucleoside at position −2 (N₋₂ of 5′- . . . N₊₂N₊₁N₀N₋₁N₋₂ . . . -3′) is a bicyclic sugar or a 2′-OR modified sugar, wherein R is optionally substituted C₁₋₆ aliphatic.
1939. The oligonucleotide of any one of the preceding Embodiments, wherein the sugar of the nucleoside at position −2 (N₋₂ of 5′- . . . N₊₂N₊₁N₀N₋₁N₋₂ . . . -3′) is a bicyclic sugar.
1940. The oligonucleotide of any one of Embodiments 1-1938, wherein the sugar of the nucleoside at position −2 (N₋₂ of 5′- . . . N₊₂N₊₁N₀N₋₁N₋₂ . . . -3′) is a 2′-OR modified sugar, wherein R is optionally substituted C₁₋₆ aliphatic.
1941. The oligonucleotide of any one of Embodiments 1-1938, wherein the sugar of the nucleoside at position −2 (N₋₂ of 5′- . . . N₊₂N₊₁N₀N₋₁N₋₂ . . . -3′) is a 2′-OMe modified sugar.
1942. The oligonucleotide of any one of Embodiments 1-1938, wherein the sugar of the nucleoside at position −2 (N₋₂ of 5′- . . . N₊₂N₊₁N₀N₋₁N₋₂ . . . -3′) is a 2′-MOE modified sugar.
1943. The oligonucleotide of any one of Embodiments 1-1938, wherein the sugar of the nucleoside at position −2 (N₋₂ of 5′- . . . N₊₂N₊₁N₀N₋₁N₋₂ . . . -3′) is a 2′-MOE modified sugar.
1944. The oligonucleotide of any one of the preceding Embodiments, wherein the sugar of the nucleoside at position −3 (N₋₃ of 5′- . . . N₊₂N₊₁N₀N₋₁N₋₂N₋₃ . . . -3′) is a 2′-F modified sugar.
1945. The oligonucleotide of any one of the preceding Embodiments, wherein each sugar of a nucleoside after N₋₃ (e.g., N₋₄, N₋₅, N₋₆, etc.) is independently a sugar that can increase stability.
1946. The oligonucleotide of any one of the preceding Embodiments, wherein each sugar of a nucleoside after N₋₃ (e.g., N₋₄, N₋₅, N₋₆, etc.) is independently a bicyclic sugar or a 2′-OR modified sugar, wherein R is optionally substituted C₁₋₆ aliphatic.
1947. The oligonucleotide of any one of the preceding Embodiments, wherein a sugar of a nucleoside after N₋₃ (e.g., N₋₄, N₋₅, N₋₆, etc.) is a bicyclic sugar.
1948. The oligonucleotide of any one of the preceding Embodiments, wherein a sugar of a nucleoside after N₋₃ (e.g., N₋₄, N₋₅, N₋₆, etc.) is a 2′-OR modified sugar, wherein R is optionally substituted C₁₋₆ aliphatic.
1949. The oligonucleotide of any one of the preceding Embodiments, wherein a sugar of a nucleoside after N₋₃ (e.g., N₋₄, N₋₅, N₋₆, etc.) is a 2′-OMe modified sugar.
1950. The oligonucleotide of any one of the preceding Embodiments, wherein a sugar of a nucleoside after N₋₃ (e.g., N₋₄, N₋₅, N₋₆, etc.) is a 2′-MOE modified sugar.
1951. The oligonucleotide of any one of Embodiments 1-1946, wherein each sugar of a nucleoside after N₋₃ (e.g., N₋₄, N₋₅, N₋₆, etc.) is independently a 2′-OR modified sugar, wherein R is optionally substituted C₁₋₆ aliphatic.
1952. The oligonucleotide of any one of Embodiments 1-1946, wherein each sugar of a nucleoside after N₋₃ (e.g., N₋₄, N₋₅, N₋₆, etc.) is independently a 2′-OMe modified sugar.
1953. The oligonucleotide of any one of Embodiments 1-1946, wherein each sugar of a nucleoside after N₋₃ (e.g., N₋₄, N₋₅, N₋₆, etc.) is independently a 2′-MOE modified sugar.
1954. The oligonucleotide of any one of the preceding Embodiments, wherein each internucleotidic linkage bonded to N₊₁ or N₀ is independently a phosphorothioate internucleotidic linkage.
1955. The oligonucleotide of any one of the preceding Embodiments, wherein each internucleotidic linkage bonded to N₊₁ or N₀ is independently a Sp phosphorothioate internucleotidic linkage.
1956. The oligonucleotide of any one of the preceding Embodiments, wherein the internucleotidic linkage between N₋₁ and N₋₂ is a non-negatively charged internucleotidic linkage.
1957. The oligonucleotide of any one of the preceding Embodiments, wherein the internucleotidic linkage between N₋₁ and N₋₂ is a neutral internucleotidic linkage.
1958. The oligonucleotide of any one of the preceding Embodiments, wherein the internucleotidic linkage between N₋₁ and N₋₂ is phosphoryl guanidine internucleotidic linkage.
1959. The oligonucleotide of any one of the preceding Embodiments, wherein the internucleotidic linkage between N₋₁ and N₋₂ is n004, n008, n025, n026.
1960. The oligonucleotide of any one of the preceding Embodiments, wherein the internucleotidic linkage between N₋₁ and N₋₂ is n001.
1961. The oligonucleotide of any one of the preceding Embodiments, wherein the internucleotidic linkage between N₋₁ and N₋₂ is chirally controlled and is Rp.
1962. The oligonucleotide of any one of Embodiments 1-1960, wherein the internucleotidic linkage between N₋₁ and N₋₂ is chirally controlled and is Sp.
1963. The oligonucleotide of any one of the preceding Embodiments, wherein the internucleotidic linkage between N₋₂ and N₋₃ is a natural phosphate linkage.
1964. The oligonucleotide of any one of the preceding Embodiments, wherein each internucleotidic linkage bonded to a nucleoside after N₋₃ (e.g., N₄, N₋₅, N₋₆, etc.) is independently a phosphorothioate internucleotidic linkage except the first internucleotidic linkage from the 3′-end.
1965. The oligonucleotide of Embodiment 1964, wherein the phosphorothioate internucleotidic linkage is chirally controlled and is Sp.
1966. The oligonucleotide of any one of the preceding Embodiments, wherein the first domain is or comprises a second domain described in any one of Embodiments 312-1965.
1967. The oligonucleotide of any one of the preceding Embodiments, wherein the first domain comprises 5′-N₁N₀N₋₁-3′ wherein each of N₁, N₀, and N₋₁ is independently a nucleoside.
1968. The oligonucleotide of any one of the preceding Embodiments, wherein the first domain comprises 5′-N₁N₀N₋₁-3′ wherein each of N₁, N₀, and N₋₁ is independently a nucleoside, and No is opposite to a target adenosine.
1969. The oligonucleotide of any one of the preceding Embodiments, wherein the first domain comprises 5′-N₁N₀N₋₁-3′ as described in any one of Embodiments 857-1965.
1970. The oligonucleotide of any one of the preceding Embodiments, capable of editing a target adenosine opposite to a nucleoside in the first domain.
1971. The oligonucleotide of any one of the preceding Embodiments, capable of editing a target adenosine opposite to a nucleoside in the second domain.
1972. The oligonucleotide of any one of the preceding Embodiments, wherein a bicyclic sugar is a LNA sugar or a cEt sugar.
1973. The oligonucleotide of any one of the preceding Embodiments, wherein the oligonucleotide comprises 1-10 (e.g., 1, 2, 3, 4, 5, 6, 7, 8, 9, or 10) natural phosphate linkages.
1974. The oligonucleotide of any one of the preceding Embodiments, wherein the oligonucleotide comprises no more than 5 (e.g., 1, 2, 3, 4, or 5) natural phosphate linkages.
1975. The oligonucleotide of any one of the preceding Embodiments, wherein the oligonucleotide comprises no more than 10 (e.g., 1, 2, 3, 4, 5, 6, 7, 8, 9, 10) n001.
1976. The oligonucleotide of any one of the preceding Embodiments, wherein the oligonucleotide comprises no more than 10 (e.g., 1, 2, 3, 4, 5, 6, 7, 8, 9, 10) phosphoryl guanidine internucleotidic linkages.
1977. The oligonucleotide of any one of the preceding Embodiments, wherein each sugar is independently selected from a natural DNA sugar, a natural RNA sugar, a 2′-F modified sugar, and a 2′-OR modified sugar wherein R is optionally substituted C₁₋₆ aliphatic.
1978. The oligonucleotide of any one of the preceding Embodiments, wherein each sugar is independently selected from a natural DNA sugar, a natural RNA sugar, a 2′-F modified sugar, and a 2′-OMe or a 2′-MOE modified sugar.
1979. The oligonucleotide of any one of the preceding Embodiments, wherein each internucleotidic linkage is independently selected from a natural phosphate linkage, a non-negatively charged internucleotidic linkage, and a phosphorothioate internucleotidic linkage.
1980. The oligonucleotide of any one of the preceding Embodiments, wherein each internucleotidic linkage is independently selected from a natural phosphate linkage, a neutral internucleotidic linkage, and a phosphorothioate internucleotidic linkage.
1981. The oligonucleotide of any one of the preceding Embodiments, wherein each internucleotidic linkage is independently selected from a natural phosphate linkage, a phosphoryl guanidine internucleotidic linkage, and a phosphorothioate internucleotidic linkage.
1982. The oligonucleotide of any one of the preceding Embodiments, wherein each internucleotidic linkage is independently selected from a natural phosphate linkage, n001, and a phosphorothioate internucleotidic linkage.
1983. The oligonucleotide of any one of the preceding Embodiments, wherein the oligonucleotide is in a salt form.
1984. The oligonucleotide of any one of the preceding Embodiments, wherein the oligonucleotide is in a pharmaceutically acceptable salt form.
1985. The oligonucleotide of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 80%, 85%, 90%, 91%, 92%, 93%, 94%, 95%, 96%, 97%, 98% or 99%.
1986. The oligonucleotide of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 90%, 91%, 92%, 93%, 94%, 95%, 96%, 97%, 98% or 99%.
1987. The oligonucleotide of any one of the preceding Embodiments, wherein diastereomeric excess of each phosphorothioate linkage phosphorus is independently about or at least about 80%, 85%, 90%, 91%, 92%, 93%, 94%, 95%, 96%, 97%, 98% or 99%.
1988. The oligonucleotide of any one of the preceding Embodiments, wherein diastereomeric excess of each phosphorothioate linkage phosphorus is independently about or at least about 90%, 91%, 92%, 93%, 94%, 95%, 96%, 97%, 98% or 99%.
1989. The oligonucleotide of any one of the preceding Embodiments, wherein diastereomeric excess of each chiral linkage phosphorus centers is independently about or at least about 80%, 85%, 90%, 91%, 92%, 93%, 94%, 95%, 96%, 97%, 98% or 99%.
1990. The oligonucleotide of any one of the preceding Embodiments, wherein diastereomeric excess of each chiral linkage phosphorus centers is independently about or at least about 90%, 91%, 92%, 93%, 94%, 95%, 96%, 97%, 98% or 99%.
1991. The oligonucleotide of any one of the preceding Embodiments, wherein the oligonucleotide has a purity of about 10%-100% (e.g., about 10%-95%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or about or at least about 10%, 20%, 30%, 40%, 50%, 60%, 65%, 70%, 75%, 80%, 85%, 90%, 95%, or 100%, etc.).
1992. The oligonucleotide of any one of the preceding Embodiments, wherein the oligonucleotide has a purity of about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.).
1993. A pharmaceutical composition which comprises or delivers an effective amount of an oligonucleotide of any one of the preceding Embodiments or a pharmaceutically acceptable salt thereof and a pharmaceutically acceptable carrier.
1994. An oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein each oligonucleotide of the plurality is independently an oligonucleotide of any one of the preceding Embodiments or an acid, base, or salt form thereof.
   1995. An oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein each oligonucleotide of the plurality is independently an oligonucleotide of any one of Embodiments 2108-2133, or an acid, base, or salt form thereof.
   1996. An oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the common base sequence is complementary to a base sequence of a portion of a nucleic acid which portion comprises a target adenosine.
   1997. The composition of Embodiment 1996, wherein the common base sequence is complementary to a base sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
   1998. The composition of Embodiment 1996, wherein the common base sequence is complementary to a base sequence of a portion of a nucleic acid with 0-5 mismatches which are not Watson-Crick base pairs.
   1999. The composition of Embodiment 1996, wherein the common base sequence is 100% complementary to a base sequence of a portion of a nucleic acid across the length of the common base sequence except the nucleoside opposite to a target adenosine.
   2000. The composition of Embodiment 1996, wherein the common base sequence is 100% complementary to a base sequence of a portion of a nucleic acid across the length of the common base sequence.
   2001. The composition of any one of Embodiments 1994-2000, wherein the composition can edit a target A to I when contacted with a nucleic acid in a system expressing ADAR.
   2002. The composition of any one of Embodiments 1994-2001, wherein the target adenosine is a G to A mutation associated with a condition, disorder or disease.
   2003. The composition of any one of Embodiments 1994-2002, wherein oligonucleotides of the plurality share the same base and sugar modifications.
   2004. The composition of any one of Embodiments 1994-2003, wherein oligonucleotides of the plurality share the same pattern of backbone chiral centers.
   2005. The composition of any one of Embodiments 1994-2004, wherein the composition is enriched for oligonucleotides of the plurality compared to a stereorandom preparation of the oligonucleotides wherein no internucleotidic linkages are chirally controlled.
   2006. The composition of any one of Embodiments 1994-2004, wherein a non-random level of all oligonucleotides in the composition that share the common base sequence and the same base and sugar modifications are oligonucleotides of the plurality.
   2007. The composition of any one of Embodiments 1994-2004, wherein a non-random level of all oligonucleotides in the composition that share the common base sequence are oligonucleotides of the plurality.
   2008. The composition of any one of Embodiments 1994-2007, wherein oligonucleotides of the plurality are of the same oligonucleotide or one or more pharmaceutically acceptable salts thereof.
   2009. The composition of any one of Embodiments 1994-2007, wherein oligonucleotides of the plurality are one or more pharmaceutically acceptable salts of the same acid-form oligonucleotide.
   2010. The composition of any one of Embodiments 1994-2007, wherein oligonucleotides of the plurality are of the same constitution.
   2011. The composition of Embodiment 2010, wherein anon-random level of all oligonucleotides in the composition that share the same base sequence as oligonucleotides of the plurality are oligonucleotides of the plurality.
   2012. The composition of Embodiment 2010, wherein anon-random level of all oligonucleotides in the composition that share the same constitution are oligonucleotides of the plurality.
   2013. The composition of any one of Embodiments 1994-2007, wherein oligonucleotides of the plurality are of the same structure.
   2014. The composition of any one of Embodiments 1994-2013, wherein oligonucleotides of the plurality are sodium salts.
   2015. The composition of any one of Embodiments 1994-2014, wherein oligonucleotides of the plurality share the same linkage phosphorus stereochemistry at 10 or more chiral internucleotidic linkages.
   2016. The composition of any one of Embodiments 1994-2015, wherein oligonucleotides of the plurality share the same linkage phosphorus stereochemistry at each phosphorothioate internucleotidic linkages.
   2017. The composition of any one of Embodiments 1994-2016, wherein oligonucleotides of the plurality do not share the same linkage phosphorus stereochemistry at one or more or any non-negatively charged internucleotidic linkages.
   2018. An oligonucleotide composition comprising one or more pluralities of oligonucleotides, wherein oligonucleotides of each plurality independently share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein each oligonucleotide of the plurality is independently an oligonucleotide of any one of the preceding Embodiments or an acid, base, or salt form thereof.
   2019. An oligonucleotide composition comprising one or more pluralities of oligonucleotides, wherein oligonucleotides of each plurality independently share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15. 16, 17, 18, 19, 20, 21, 22, 23, 24, or 25 or more) chiral internucleotidic linkages (“chirally controlled internucleotidic linkages”);
wherein each oligonucleotide of the plurality is independently an oligonucleotide of any one of the preceding Embodiments and Embodiments 2108-2133, or an acid, base, or salt form thereof.
   2020. An oligonucleotide composition comprising one or more pluralities of oligonucleotides, wherein oligonucleotides of each plurality independently share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein each oligonucleotide of the plurality is independently an oligonucleotide of any one of Embodiments 2108-2133, or an acid, base, or salt form thereof.
   2021. An oligonucleotide composition comprising one or more pluralities of oligonucleotides, wherein oligonucleotides of each plurality independently share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1,2, 3, 4, 5, 6,7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the common base sequence of each plurality is independently complementary to a base sequence of a portion of a nucleic acid which portion comprises a target adenosine.
   2022. The composition of Embodiment, wherein the common base sequence is complementary to abase sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
   2023. The composition of Embodiment 2022, wherein the common base sequence of each plurality is independently complementary to a base sequence of a portion of a nucleic acid with 0-5 mismatches which are not Watson-Crick base pairs.
   2024. The composition of Embodiment 2022, wherein the common base sequence of each plurality is independently 100% complementary to a base sequence of a portion of a nucleic acid across the length of the common base sequence except the nucleoside opposite to a target adenosine.
   2025. The composition of Embodiment 2022, wherein the common base sequence of each plurality is independently 100% complementary to a base sequence of a portion of a nucleic acid across the length of the common base sequence.
   2026. The composition of any one of Embodiments 2018-2025, wherein each plurality of oligonucleotides can independently edit a target A to I when contacted with a nucleic acid in a system expressing ADAR.
   2027. The composition of any one of Embodiments 2018-2026, wherein a target adenosine is a G to A mutation associated with a condition, disorder or disease.
   2028. The composition of any one of Embodiments 2018-2027, wherein the composition comprises two or more (e.g., 2, 3, 4, 5, 6, 7, 8, 9, 10, or more) pluralities of oligonucleotides.
   2029. The composition of any one of Embodiments 2018-2028, wherein common base sequences of at least two pluralities are different.
   2030. The composition of any one of Embodiments 2018-2029, wherein no two pluralities of oligonucleotides share the same common base sequence.
   2031. The composition of any one of Embodiments 2018-2030, wherein at least two pluralities of oligonucleotides target different adenosine.
   2032. The composition of any one of Embodiments 2018-2031, wherein no two pluralities of oligonucleotides target the same adenosine.
   2033. The composition of any one of Embodiments 2018-2032, wherein at least two pluralities of oligonucleotides target different transcripts.
   2034. The composition of any one of Embodiments 2018-2033, wherein no two pluralities of oligonucleotides target the same transcript.
   2035. The composition of any one of Embodiments 2018-2034, wherein at least two plurality of oligonucleotides target adenosine residues in transcripts from different polynucleotides.
   2036. The composition of any one of Embodiments 2018-2037, wherein no two pluralities of oligonucleotides target transcripts from the same polynucleotide.
   2037. The composition of any one of Embodiments 2018-2036, wherein at least two plurality of oligonucleotides target adenosine residues in transcripts from different genes.
   2038. The composition of any one of Embodiments 2018-2037, wherein no two pluralities of oligonucleotides target transcripts from the same gene.
   2039. The composition of any one of Embodiments 2018-2038, wherein oligonucleotides of each plurality independently share the same base and sugar modifications within the plurality.
   2040. The composition of any one of Embodiments 2018-2039, wherein oligonucleotides of each plurality independently share the same pattern of backbone chiral centers within the plurality.
   2041. The composition of any one of Embodiments 2018-2040, wherein for each plurality independently, the composition is enriched for oligonucleotides of that plurality compared to a stereorandom preparation of oligonucleotides of that plurality wherein no internucleotidic linkages are chirally controlled.
   2042. The composition of any one of Embodiments 2018-2041, wherein for each plurality independently, a non-random level of all oligonucleotides in the composition that share the common base sequence and the same base and sugar modifications are oligonucleotides of the plurality.
   2043. The composition of any one of Embodiments 2018-2041, wherein for each plurality independently, a non-random level of all oligonucleotides in the composition that share the common base sequence are oligonucleotides of the plurality.
   2044. The composition of any one of Embodiments 2018-2043, wherein for each plurality independently, oligonucleotides of the plurality are of the same oligonucleotide or one or more pharmaceutically acceptable salts thereof.
   2045. The composition of any one of Embodiments 2018-2044, wherein for each plurality independently, oligonucleotides of the plurality are one or more pharmaceutically acceptable salts of the same acid-form oligonucleotide.
   2046. The composition of any one of Embodiments 2018-2043, wherein for each plurality independently, oligonucleotides of the plurality are of the same constitution.
   2047. The composition of Embodiment 2046, wherein for each plurality independently, a non-random level of all oligonucleotides in the composition that share the same base sequence as oligonucleotides of the plurality are oligonucleotides of the plurality.
   2048. The composition of Embodiment 2046, wherein for each plurality independently, a non-random level of all oligonucleotides in the composition that share the same constitution are oligonucleotides of the plurality.
   2049. The composition of any one of Embodiments 2018-2048, wherein for one or two or all pluralities independently, oligonucleotides of the plurality are of the same structure.
   2050. The composition of any one of Embodiments 2018-2049, wherein for one or two or all pluralities independently, oligonucleotides of the plurality are each independently a pharmaceutically acceptable salt form.
   2051. The composition of any one of Embodiments 2018-2049, wherein for one or two or all pluralities independently, oligonucleotides of the plurality are sodium salts.
   2052. The composition of any one of Embodiments 2018-2051, wherein for one or two or all pluralities independently, oligonucleotides of the plurality share the same linkage phosphorus stereochemistry at 10 or more chiral internucleotidic linkages.
   2053. The composition of any one of Embodiments 2018-2052, wherein for each plurality independently, oligonucleotides of the plurality share the same linkage phosphorus stereochemistry at 10 or more chiral internucleotidic linkages.
   2054. The composition of any one of Embodiments 2018-2053, wherein for one or two or all pluralities independently, oligonucleotides of the plurality share the same linkage phosphorus stereochemistry at each phosphorothioate internucleotidic linkages.
   2055. The composition of any one of Embodiments 2018-2054, wherein for each plurality independently, oligonucleotides of the plurality share the same linkage phosphorus stereochemistry at each phosphorothioate internucleotidic linkages.
   2056. The composition of any one of Embodiments 2018-2055, wherein for one or two or all pluralities independently, oligonucleotides of the plurality do not share the same linkage phosphorus stereochemistry at one or more or any non-negatively charged internucleotidic linkages.
   2057. The composition of any one of Embodiments 2018-2056, wherein for each plurality independently, oligonucleotides of the plurality do not share the same linkage phosphorus stereochemistry at one or more or any non-negatively charged internucleotidic linkages.
   2058. A composition comprising a plurality of oligonucleotides which are of a particular oligonucleotide type characterized by:
a) a common base sequence;
b) a common pattern of backbone linkages;
c) a common pattern of backbone chiral centers;
d) a common pattern of backbone phosphorus modifications;
which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and
wherein each oligonucleotide of the plurality is independently an oligonucleotide of any one of the preceding Embodiments or an acid, base, or salt form thereof.
   2059. A composition comprising a plurality of oligonucleotides which are of a particular oligonucleotide type characterized by:
a) a common base sequence;
b) a common pattern of backbone linkages;
c) a common pattern of backbone chiral centers;
d) a common pattern of backbone phosphorus modifications;
which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and
wherein each oligonucleotide of the plurality is independently an oligonucleotide of any one of Embodiments 2108-2133, or an acid, base, or salt form thereof.
   2060. A composition comprising a plurality of oligonucleotides which are of a particular oligonucleotide type characterized by:
a) a common base sequence;
b) a common pattern of backbone linkages;
c) a common pattern of backbone chiral centers;
d) a common pattern of backbone phosphorus modifications;
which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and
wherein the common base sequence is complementary to a base sequence of a portion of a nucleic acid which portion comprises a target adenosine.
   2061. The composition of Embodiment 2060, wherein the common base sequence is complementary to a base sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
   2062. The composition of Embodiment 2060, wherein the common base sequence is complementary to a base sequence of a portion of a nucleic acid with 0-5 mismatches which are not Watson-Crick base pairs.
   2063. The composition of Embodiment 2060, wherein the common base sequence is 100% complementary to a base sequence of a portion of a nucleic acid across the length of the common base sequence except the nucleoside opposite to a target adenosine.
   2064. The composition of Embodiment 2060, wherein the common base sequence is 100% complementary to a base sequence of a portion of a nucleic acid across the length of the common base sequence.
   2065. The composition of any one of Embodiments 2058-2064, wherein the composition can edit a target A to I when contacted with a nucleic acid in a system expressing ADAR.
   2066. The composition of any one of Embodiments 2058-2065, wherein the target adenosine is a G to A mutation associated with a condition, disorder or disease.
   2067. The composition of any one of Embodiments 2058-2066, wherein the composition is enriched, relative to a substantially racemic preparation of oligonucleotides having the same common base sequence, pattern of backbone linkages and pattern of backbone phosphorus modifications, for oligonucleotides of the particular oligonucleotide type.
   2068. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-1984.
   2069. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-1984, wherein at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%0, 90%, 91%, 92%, 93%, 94%, 95%, 96%, 97%, 98%, or 99% of all oligonucleotides in the composition that share the base sequence of a the particular oligonucleotide are oligonucleotide of the plurality.
   2070. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-1984, wherein at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the constitution of the particular oligonucleotide or a salt thereof are oligonucleotide of the plurality.
   2071. A composition comprising a plurality of oligonucleotides, wherein each oligonucleotides of the plurality is independently a particular oligonucleotide or a salt thereof, wherein the particular oligonucleotide is an oligonucleotide of Table 1.
   2072. The composition of any one of Embodiments 2058-2071, wherein anon-random level of all oligonucleotides in the composition that share the common base sequence are oligonucleotides of the plurality.
   2073. The composition of any one of Embodiments 1994-2072, wherein the level of oligonucleotides of a plurality in oligonucleotides in the composition that share the common base sequence of the plurality is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
   2074. The composition of any one of Embodiments 1994-2072, wherein for each plurality of oligonucleotides, the level of oligonucleotides of the plurality in oligonucleotides in the composition that share the common base sequence of the plurality is independently about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
   2075. The composition of any one of Embodiments 1994-2072, wherein the level of oligonucleotides of a plurality in oligonucleotides in the composition that share the common constitution of the plurality is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
   2076. The composition of any one of Embodiments 1994-2072, wherein for each plurality of oligonucleotides, the level of oligonucleotides of the plurality in oligonucleotides in the composition that share the common constitution of the plurality is independently about or at least about (DS)^{nc}, wherein DS is about 85%-100% (e.g., about 85%, 86%, 87%, 88%, 89%, 90%, 9l %, 92%, 93%, 94%, 95%, 96%, 97%, 98%, 99% or 99.5% or more) and nc is the number of chirally controlled internucleotidic linkages.
   2077. The composition of any one of Embodiments 1994-2076, wherein DS is about 90%-100% (e.g.. about 90%, 91%, 92%, 93%, 94%, 95%, 96%, 97%, 98%, 99% or 99.5% or more).
   2078. The composition of any one of Embodiments 2073-2077, wherein nc is about 5-40 (e.g., about 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) or more.
   2079. The composition of any one of Embodiments 1994-2072, wherein the level is at least about 10%-100%, or at least about 10%, 20%, 30%, 40%, 50%, 60%, 65%, 70%, 75%, 80%, 85%, 90%, or 95%,
   2080. The composition of any one of Embodiments 1994-2072, wherein the level is at least about 50%-100%, or at least about 50%, 60%, 65%, 70%, 75%, 80%, 85%, 90%, or 95%.
   2081. A composition comprising a particular oligonucleotide, wherein about 10%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%0, % 60%, 65%, 70%, 75%, 80%, 85%, 90%, 95%, or 100%, etc.) of all oligonucleotides in the composition that share the base sequence of the oligonucleotide are independently the particular oligonucleotide or a salt thereof.
   2082. A composition comprising a particular oligonucleotide, wherein about 30%-90% of all oligonucleotides in the composition that share the base sequence of the oligonucleotide are independently the particular oligonucleotide or a salt thereof.
   2083. A composition comprising a particular oligonucleotide, wherein about 40%-90% of all oligonucleotides in the composition that share the base sequence of the oligonucleotide are independently the particular oligonucleotide or a salt thereof
   2084. The composition of any one of Embodiments 2081-2083, wherein the particular oligonucleotide is an oligonucleotide of any one of Embodiments 1-1992.
   2085. The composition of any one of Embodiments 2081-2084, wherein the particular oligonucleotide is an oligonucleotide selected from Table 1.
   2086. The composition of any one of Embodiments 2081-2085, wherein the particular oligonucleotide comprises about or at least about 5, 6, 7, 8, 9, 10, 11, 12, 13, 14, 15, 16, 17, 18, 19, 20, 21, 22, 23, 24, 25, 26, 27, 28, 29, 30 or more chiral internucleotidic linkages.
   2087. The composition of any one of Embodiments 2081-2086, wherein each salt is independently a pharmaceutically acceptable salt.
   2088. The composition of any one of Embodiments 1994-2087, wherein when the composition is contacted with a sample comprising the target nucleic acid and an adenosine deaminase, the target adenosine residue is modified.
   2089. The composition of Embodiment 2088, wherein the modification is or comprises modification performed by ADAR1.
   2090. The composition of Embodiment 2088 or 2089, wherein the modification is or comprises modification performed by ADAR2.
   2091. The composition of any one of Embodiments 2088-2090, wherein the modification is performed in vitro.
   2092. The composition of any one of Embodiments 2088-2090, wherein the sample is a cell.
   2093. The composition of any one of Embodiments 2088-2092, wherein the target adenosine is converted into inosine.
   2094. The composition of any one of Embodiments 2088-2093, wherein the target adenosine is modified to a greater degree than that is observed with a comparable reference oligonucleotide composition.
   2095. The composition of Embodiment 2094, wherein the reference oligonucleotide composition comprises no or a lower level of oligonucleotides of the plurality.
   2096. The composition of any one of Embodiments 2094-2095, wherein the reference composition does not contain oligonucleotides that have the same constitution as an oligonucleotide of the plurality.
   2097. The composition of any one of Embodiments 2094-2096, wherein the reference composition does not contain oligonucleotides that have the same structure as an oligonucleotide of the plurality.
   2098. The composition of Embodiment 2094, wherein the reference oligonucleotide composition is a composition whose oligonucleotides having the same base sequence as oligonucleotides of the plurality contain a lower level of 2′-F modifications compared to oligonucleotides of the plurality.
   2099. The composition of any one of Embodiments 2094-2098, wherein the reference oligonucleotide composition is a composition whose oligonucleotides having the same base sequence as oligonucleotides of the plurality contain a lower level of 2′-OMe modifications compared to oligonucleotides of the plurality.
   2100. The composition of any one of Embodiments 2094-2099, wherein the reference oligonucleotide composition is a composition whose oligonucleotides having the same base sequence as oligonucleotides of the plurality have a different sugar modification pattern compared to oligonucleotides of the plurality.
   2101. The composition of any one of Embodiments 2094-2100, wherein the reference oligonucleotide composition is a composition whose oligonucleotides having the same base sequence as oligonucleotides of the plurality contain a lower level of modified internucleotidic linkages compared to oligonucleotides of the plurality.
   2102. The composition of any one of Embodiments 2094-2101, wherein the reference oligonucleotide composition is a composition whose oligonucleotides having the same base sequence as oligonucleotides of the plurality contain a lower level of phosphorothioate internucleotidic linkages compared to oligonucleotides of the plurality.
   2103. The composition of any one of Embodiments 2094-2102, wherein the reference composition is a stereorandom oligonucleotide composition.
   2104. The composition of Embodiment 2094, wherein the reference composition is a stereorandom oligonucleotide composition of oligonucleotides of the same constitution as oligonucleotides of the plurality.
   2105. The composition of any one of the preceding Embodiments, wherein the composition does not cause significant degradation of the nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%0, 40%, 50%, 60% 65%, 70%, 75%, 80%, 85%, 90%, 95%, or 100%, etc.)).
   2106. The composition of any one of the preceding Embodiments, wherein the composition does not cause significant exon skipping or altered exon inclusion in the target nucleic acid (e.g., no more than about 5%-100% (e.g., no more than about 10%-100%, 20-100%, 30%-100%, 40%-10%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%. 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).
   2107. The composition of any one of Embodiments 1994-2106, wherein the composition is a pharmaceutical composition, and further comprises a pharmaceutically acceptable carrier.
   2108. An oligonucleotide, wherein the oligonucleotide is otherwise identical to an oligonucleotide of any one of the preceding Embodiments, except that at a position of a modified internucleotidic linkage is a linkage having the structure of -O⁵-P^{L}(R^{CA})-O³-, wherein:
P^{L} is P, or P(═W);
W is O, S, or W^{N};
R^{CA} is or comprises an optionally substituted or capped chiral auxiliary moiety,
O⁵ is an oxygen bonded to a 5′-carbon of a sugar, and
O is an oxygen bonded to a 3′-carbon of a sugar.
   2109. The oligonucleotide of Embodiment 2108, wherein the chiral auxiliary is removed the linkage is converted to the modified internucleotidic linkage.
   2110. The oligonucleotide of Embodiment 2108, wherein a modified internucleotidic linkage is a phosphorothioate internucleotidic linkage.
   2111. The oligonucleotide of Embodiment 2110, wherein when W is replaced with —SH and R^{CA} is replaced with 0, P^{L} has the same configuration as the linkage phosphorus of the phosphorothioate internucleotidic linkage.
   2112. The oligonucleotide of any one of Embodiments 2108-2111, wherein a modified internucleotidic linkage is a neutral internucleotidic linkage.
   2113. The oligonucleotide of any one of Embodiments 2108-2111, wherein a modified internucleotidic linkage is a phosphoryl guanidine internucleotidic linkage.
   2114. The oligonucleotide of any one of Embodiments 2108-2111, wherein a modified internucleotidic linkage is n004, n008, n025, n026.
   2115. The oligonucleotide of any one of Embodiments 2108-2111, wherein a modified internucleotidic linkage is n001.
   2116. The oligonucleotide of any one of Embodiments 2108-2115, wherein at each position of a phosphorothioate internucleotidic linkage is independently a linkage having the structure of -O⁵-P^{L}(W)(R^{CA})-O³-.
   2117. The oligonucleotide of any one of Embodiments 2108-2115, wherein at each position of a modified internucleotidic linkage is independently a linkage having the structure of -O⁵-P^{L}(W)(R^{CA})-O³-.
   2118. The oligonucleotide of any one of Embodiments 2108-2117, wherein one or each W is S.
   2119. The oligonucleotide of any one of Embodiments 2108-2118, wherein one and only one P^{L} is P.
   2120. The oligonucleotide of any one of Embodiments 2108-2119, wherein each R^{CA} is independently

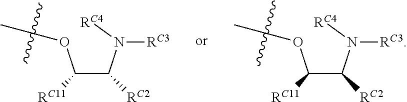

2121. The oligonucleotide of any one of Embodiments 2108-2119, wherein each R^{CA} is independently

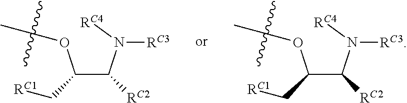

wherein R^{C1} is R -Si(R)₃ or —SO₂R, R^{C2} and R^{C3} are taken together with their intervening atoms to form an optionally substituted 3-7 membered saturated or partially unsaturated ring having, in addition to the nitrogen atom, 0-2 heteroatoms, R^{C4} is —H or —C(O)R′.
2122. The oligonucleotide of Embodiment 2120 or 2121, wherein in a linkage, R^{C4} is —C(O)R and P^{L} is P.
2123. The oligonucleotide of any one of Embodiments 2121-2122, wherein in a linkage, R^{C4} is —C(O)R and W is S.
2124. The oligonucleotide of any one of Embodiments 2121-2123, wherein in each linkage wherein W is S, RC⁴ is —C(O)R′.
2125. The oligonucleotide of any one of Embodiments 2121-2124, wherein R^{C4} is —C(O)CH₃.
2126. The oligonucleotide of Embodiment 2121, wherein in a linkage, R^{C4} is —H and P^{L} is P.
2127. The oligonucleotide of any one of Embodiments 2121-2126, wherein R^{C2} and R^{C3} are taken together with their intervening atoms to form an optionally substituted 5-membered ring having no heteroatoms in addition to the nitrogen atom.
2128. The oligonucleotide of any one of Embodiments 2121-2127, wherein each R^{CA} is independently

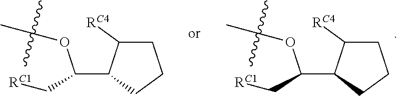

2129. The oligonucleotide of any one of Embodiments 2121-2128, wherein R^{C1} is -SiPh₂Me.
2130. The oligonucleotide of any one of Embodiments 2121-2128, wherein R^{C1} is —SO₂R.
2131. The oligonucleotide of any one of Embodiments 2121-2128, wherein R^{C1} is —SO₂R wherein R is optionally substituted C₁₋₁₀ aliphatic.
2132. The oligonucleotide of any one of Embodiments 2121-2128, wherein R^{C1} is —SO₂R wherein R is optionally substituted phenyl.
2133. The oligonucleotide of any one of Embodiments 2121-2128, wherein R^{C1} is —SO₂R, wherein R is phenyl.
2134. A phosphoramidite, wherein the nucleobase of the phosphoramidite is a nucleobase of any one of Embodiments 1-1992 or a tautomer thereof, wherein the nucleobase or tautomer thereof is optionally substituted or protected.
2135. A phosphoramidite, wherein the nucleobase is or comprises Ring BA, wherein Ring BA has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected.
2136. The phosphoramidite of any one of Embodiments 2134-2135, wherein the sugar of the phosphoramidite is a sugar of any one of Embodiments 1-1992, wherein the sugar is optionally protected.
2137. The phosphoramidite of any one of Embodiments 2134-2136, wherein the phosphoramidite has the structure of R^{NS}—P(OR)N(R)₂, wherein R^{NS} is a optionally protected nucleoside moiety, and each R is as described herein.
2138. The phosphoramidite of any one of Embodiments 2134-2136, wherein the phosphoramidite has the structure of R^{NS}-P(OCH₂CH₂CN)N(i-Pr)₂.
2139. The phosphoramidite of any one of Embodiments 2134-2136, wherein the phosphoramidite comprises a chiral auxiliary moiety, wherein the phosphorus is bonded to an oxygen and a nitrogen atom of the chiral auxiliary moiety.
2140. The phosphoramidite of any one of Embodiments 2134-2136 or 2139, wherein the phosphoramidite has the structure of

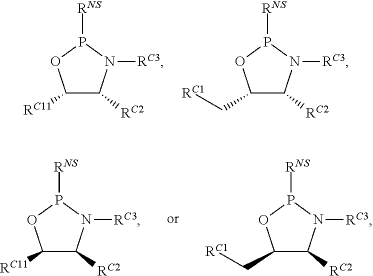

or a salt thereof.
2141. The phosphoramidite of any one of Embodiments 2134-2136 or 2139, wherein the phosphoramidite has the structure of

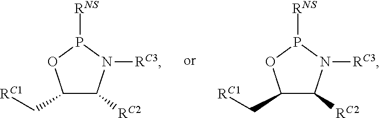

wherein R^{NS} is a optionally protected nucleoside moiety, R^{C1} is R, —Si(R)₃ or —SO₂R, R^{C2} and R^{C3} are taken together with their intervening atoms to form an optionally substituted 3-7 membered saturated or partially unsaturated ring having, in addition to the nitrogen atom, 0-2 heteroatoms.
2142. The phosphoramidite of any one of Embodiments 2140-2141, wherein R^{C2} and R^{C3} are taken together with their intervening atoms to form an optionally substituted 5-membered saturated ring having no heteroatoms in addition to the nitrogen atom.
2143. The phosphoramidite of any one of Embodiments 2140-2142, wherein the phosphoramidite has the structure of

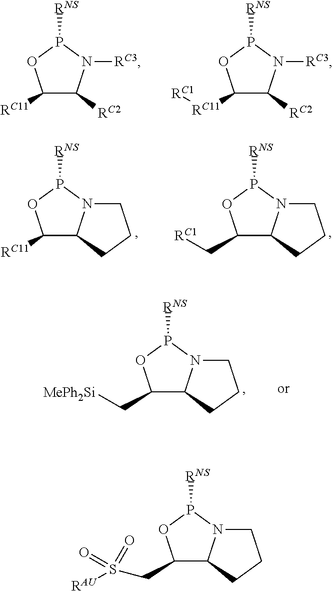

or a salt thereof.
2144. The phosphoramidite of any one of Embodiments 2140-2142, wherein the phosphoramidite has the structure of

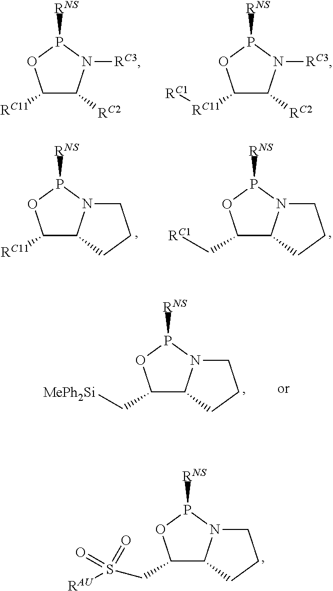

or a salt thereof.
2145. The phosphoramidite of any one of Embodiments 2140-2142, wherein the phosphoramidite has the structure of

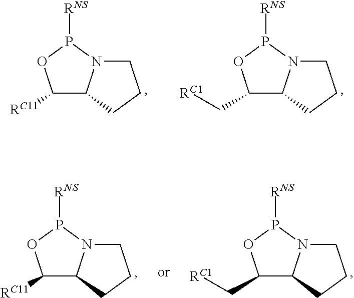

or a salt thereof.
2146. The phosphoramidite of any one of Embodiments 2140-2142, wherein the phosphoramidite has the structure of

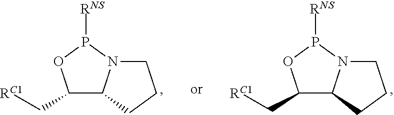

2147. The phosphoramidite of any one of Embodiments 2140-2146, wherein R^{C1} is -SiPh₂Me.
2148. The phosphoramidite of any one of Embodiments 2140-2146, wherein R^{C1} is —SO₂R.
2149. The phosphoramidite of any one of Embodiments 2140-2146, wherein R^{C1} is —SO₂R, wherein R is optionally substituted C₁₋₁₀ aliphatic.
2150. The phosphoramidite of any one of Embodiments 2140-2146, wherein R^{C1} is —SO₂R, wherein R is optionally substituted phenyl.
2151. The phosphoramidite of any one of Embodiments 2140-2146, wherein R^{C1} is —SO₂R, wherein R is phenyl.
2152. A compound having the structure of

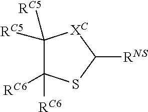

or a salt thereof, wherein R^{NS} is an optionally substituted/protected nucleoside, X^{C} is O or S, and each of R^{C5} and R^{C6} is independently R.
2153. The compound of Embodiment 2152, wherein X^{C} is O.
2154. The compound of Embodiment 2152, wherein X^{C} is S.
2155. The compound of any one of Embodiments 2152-2154, wherein one R^{C5} is not hydrogen.
2156. The compound of any one of Embodiments 2152-2155, wherein one R^{C5} is hydrogen.
2157. The compound of any one of Embodiments 2152-2156, wherein one R^{C6} is not hydrogen.
2158. The compound of any one of Embodiments 2152-2157, wherein one R^{C6} is hydrogen.
2159. The compound of any one of Embodiments 2152-2158, wherein one R^{C5} and one R^{C6} are taken together with their intervening atoms to form an optionally substituted 3-20 (e.g., 3-15, 3-10, 5-10, 3, 4, 5, 6, 7, 8, 9, 10, 11, 12, 13, 14, 15, 16, 17, 18, 19, 20) membered monocyclic, bicyclic or polycyclic ring having 0-5 heteroatoms.
2160. The compound of any one of Embodiments 2152-2158, wherein one R^{C5} and one R^{C6} are taken together with their intervening atoms to form an optionally substituted cyclohexyl ring.
2161. The compound of Embodiment 2152, wherein -X^{C}-C(R^{C5})₂-C(R^{C6})₂-S- is —OCH(CH₃)CH(CH₃)S-.
2162. The compound of Embodiment 2152, wherein -X^{C}-C(R^{C5})₂-C(R^{C6})₂-S- is —SCH(CH₃)CH(CH₃)S-.
2163. The phosphoramidite or compound of any one of Embodiments 2137-2162, wherein a hydroxyl group of R^{NS} is protected.
2164. The phosphoramidite or compound of any one of Embodiments 2137-2162, wherein a hydroxyl group of R^{NS} is protected as —ODMTr.
2165. The phosphoramidite or compound of any one of Embodiments 2137-2162, wherein the 5′—OH of R^{NS} is protected.
2166. The phosphoramidite or compound of Embodiment 2165, wherein the 5′—OH of R^{NS} is protected as -ODMTr.
2167. The phosphoramidite or compound of any one of Embodiments 2137-2166, wherein R^{NS} is an optionally substituted or protected nucleoside selected from

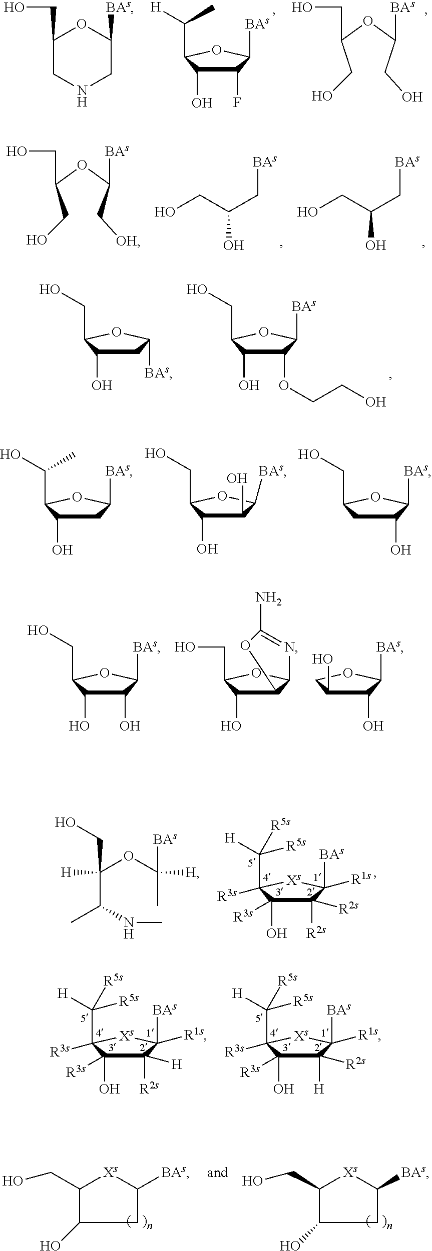

or a salt thereof, wherein BA^{s} is as described herein.
2168 The phosphoramidite or compound of any one of Embodiments 2137-2167 wherein R^{NS} is selected, from

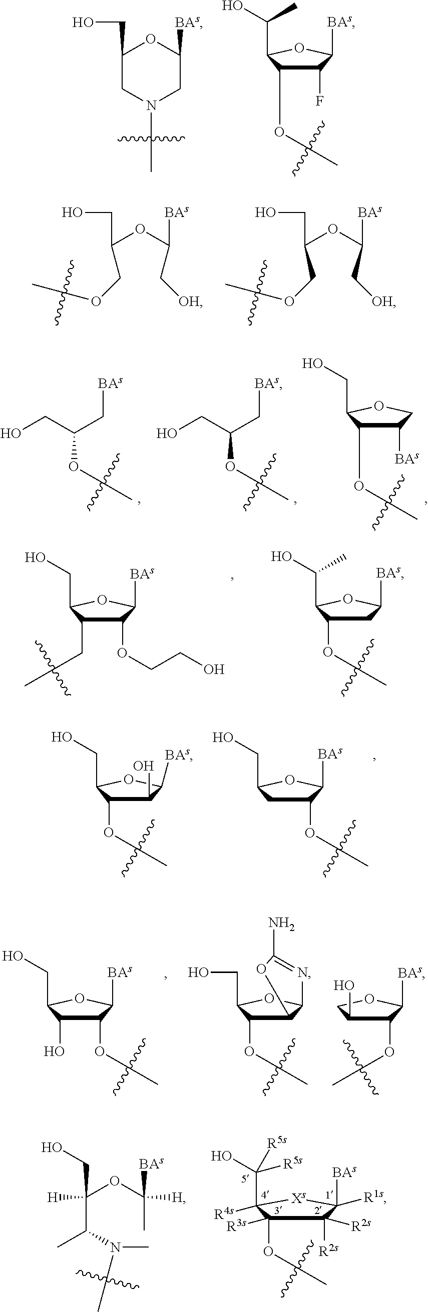

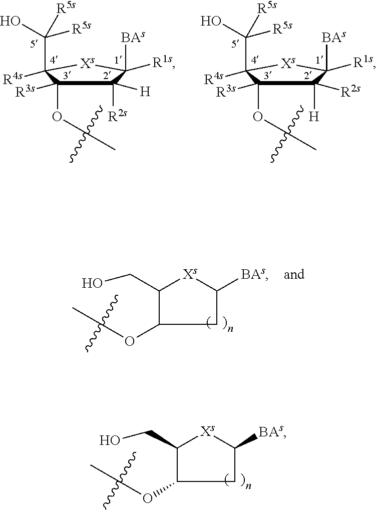

or a salt thereof, wherein BA^{s} is as described herein.
2169. The phosphoramidite or compound of any one of Embodiments 2137-2166, wherein R^{NS} is an optionally substituted or protected nucleoside selected from

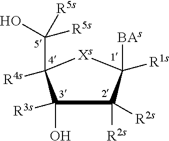

or a salt thereof.
2170. The phosphoramidite or compound of any one of Embodiments 2137-2166, wherein R^{NS} is an optionally substituted or protected nucleoside selected from

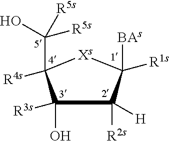

or a salt thereof.
2171. The phosphoramidite or compound of any one of Embodiments 2137-2166, wherein R^{NS} is an optionally substituted or protected nucleoside selected from

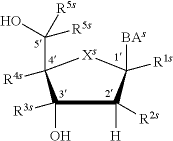

or a salt thereof.
2172. The phosphoramidite or compound of any one of Embodiments 2137-2166, wherein R^{NS} is

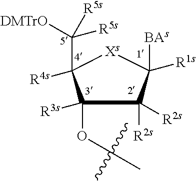

or a salt thereof, wherein BA^{s} is an optionally substituted or protected nucleobase.
2173. The phosphoramidite or compound of any one of Embodiments 2137-2166, wherein RNS is

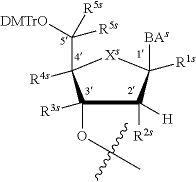

or a salt thereof, wherein BA^{s} is an optionally substituted or protected nucleobase.
2174. The phosphoramidite or compound of any one of Embodiments 2137-2166, wherein R^{NS} is

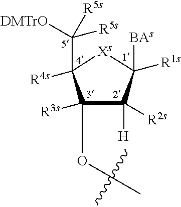

or a salt thereof, wherein BAs is an optionally substituted or protected nucleobase.
2175. The phosphoramidite or compound of any one of Embodiments 2167-2174, wherein R^{1s} is —H.
2176. The phosphoramidite or compound of any one of Embodiments 2167-2175, wherein R^{2s} is —H.
2177. The phosphoramidite or compound of any one of Embodiments 2167-2175, wherein R^{2s} is -F.
2178. The phosphoramidite or compound of any one of Embodiments 2167-2175, wherein R^{2s} is —OH.
2179. The phosphoramidite or compound of any one of Embodiments 2167-2175, wherein R₂ₛ is —OR^{ak}, wherein R^{ak} is optionally substituted C₁₋₆ aliphatic.
2180. The phosphoramidite or compound of any one of Embodiments 2167-2175, wherein R^{2s} is —OMe.
2181. The phosphoramidite or compound of any one of Embodiments 2167-2175, wherein R^{2s} is —OCH₂CH₂OCH₃.
2182. The oligonucleotide of any one of Embodiments 2167-2181, wherein R^{3s} is —H.
2183. The oligonucleotide of any one of Embodiments 2167-2182, wherein R^{4s} is —H.
2184. The oligonucleotide of any one of Embodiments 2167-2183, wherein one occurrence of R^{5s} is —H.
2185. The oligonucleotide of any one of Embodiments 2167-2184, wherein the other occurrence of R^{5s} is —H.
2186. The oligonucleotide of any one of Embodiments 2167-2184, wherein the other occurrence of R^{5s} is optionally substituted C₁₋₆ aliphatic.
2187. The phosphoramidite or compound of any one of Embodiments 2137-2166, wherein R^{NS} is an optionally substituted or protected nucleoside selected from

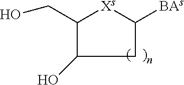

or a salt thereof.
2188. The phosphoramidite or compound of any one of Embodiments 2137-2166, wherein R^{NS} is an optionally substituted or protected nucleoside selected from

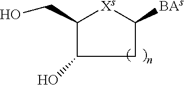

or a salt thereof.
2189. The phosphoramidite or compound of any one of Embodiments 2137-2166, wherein R^{NS} is optionally substituted or protected

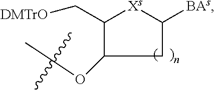

wherein X^{s} is —O—, —S— or —Se—, and n is 0, 1, 2 or 3.
2190. The phosphoramidite or compound of any one of Embodiments 2137-2166, wherein R^{NS} is optionally substituted or protected

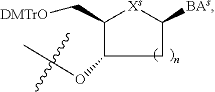

wherein X^{s} is —O—, —S— or —Se—, and n is 0, 1, 2 or 3.
2191. The phosphoramidite or compound of any one of Embodiments 2137-2166, wherein R^{NS} is

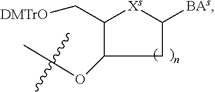

wherein X^{s} is —O—, —S— or —Se—, and n is 0, 1, 2 or 3.
2192. The phosphoramidite or compound of any one of Embodiments 2137-2166, wherein R^{NS} is

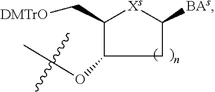

wherein X^{s} is —O—, —S— or —Se—, and n is 0, 1, 2 or 3.
2193. The phosphoramidite or compound of any one of Embodiments 2187-2192, wherein n is 0.
2194. The phosphoramidite or compound of any one of Embodiments 2187-2192, wherein n is 1.
2195. The phosphoramidite or compound of any one of Embodiments 2187-2192, wherein n is 2.
2196. The phosphoramidite or compound of any one of Embodiments 2187-2192, wherein n is 3.
2197. The phosphoramidite or compound of any one of Embodiments 2167-2196, wherein X^{s} is —O—.
2198. The phosphoramidite or compound of any one of Embodiments 2167-2196, wherein X^{s} is -S-.
2199. The phosphoramidite or compound of any one of Embodiments 2167-2196, wherein X^{s} is optionally substituted —CH₂-.
2200. The phosphoramidite or compound of any one of Embodiments 2167-2196, wherein X^{s} is —CH₂-.
2201. The phosphoramidite or compound of any one of Embodiments 2137-2200, wherein the phosphoramidite or compound comprises a nucleobase of Table BA-1 or a tautomer thereof, wherein the nucleobase or tautomer thereof is optionally substituted or protected.
2202. The phosphoramidite or compound of any one of Embodiments 2137-2200, wherein the phosphoramidite or compound comprises b014U, b015U, b004A, b005A, b006A, and b007A, or a tautomer thereof, wherein the nucleobase or tautomer thereof is optionally substituted or protected.
2203. The phosphoramidite or compound of any one of Embodiments 2137-2200, wherein the phosphoramidite or compound comprises a nucleobase of any one of Embodiments 1-1992 or a tautomer thereof, wherein the nucleobase or tautomer thereof is optionally substituted or protected.
2204. The phosphoramidite or compound of any one of Embodiments 2137-2203, wherein the phosphoramidite or compound comprises a nucleobase, wherein the nucleobase is or comprises Ring BA, wherein Ring BA has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected.
2205. The phosphoramidite or compound of any one of Embodiments 2137-2204, wherein the phosphoramidite or compound comprises a nucleobase, wherein the nucleobase is or comprises Ring BA, wherein Ring BA has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected.
2206. The phosphoramidite or compound of any one of Embodiments 2137-2205, wherein BA^{s} has the structure of BA-I, BA-I-a, BA-I-b, BA-I-c, BA-I-d, BA-II, BA-II-a, BA-II-b, BA-II-c, BA-II-d, BA-III, BA-III-a, BA-III-b, BA-III-c, BA-III-d, BA-III-e, BA-IV, BA-IV-a, BA-IV-b, BA-V, BA-V-a, BA-V-b, or BA-VI, or a tautomer of Ring BA, wherein the nucleobase is optionally substituted or protected.
2207. The phosphoramidite or compound of any one of Embodiments 2137-2203, wherein the phosphoramidite or compound comprises hypoxanthine.
2208. The phosphoramidite or compound of any one of Embodiments 2137-2203, wherein the phosphoramidite or compound comprises O⁶-protected hypoxanthine.
2209. The phosphoramidite or compound of any one of Embodiments 2137-2203, wherein the phosphoramidite or compound comprises O⁶-protected hypoxanthine, wherein the O⁶ protection group is —CH₂CH₂Si(R)₃, wherein the —CH₂CH₂- is optionally substituted and each R is not —H.
2210. The phosphoramidite or compound of any one of Embodiments 2137-2203, wherein the phosphoramidite or compound comprises O⁶-protected hypoxanthine, wherein the O⁶ protection group is —CH₂CH₂Si(Me)₃.
2211. The phosphoramidite or compound of any one of Embodiments 2137-2210, wherein the phosphoramidite or compound comprises a sugar which is a sugar of any one of Embodiments 1-1992.
2212. The phosphoramidite or compound of any one of Embodiments 2137-2166, wherein R^{NS} is an optionally substituted or protected nucleoside selected from A, T, C, G and U.
2213. The phosphoramidite or compound of any one of Embodiments 2137-2166, wherein R^{NS} is an optionally substituted or protected nucleoside selected from b001Asm15, b004A, b005A, b006A, b014U, b015U, b008Usm15, Csm19, b008Usm19.
2214. The phosphoramidite or compound of any one of Embodiments 2137-2166, wherein R^{NS} is an optionally substituted or protected nucleoside selected from b001U, b002U, b003U, b004U, b005U, b006U, b008U, b002A, b001G, b004C, b007U, b001A, b001C, b002C, b003C, b002I, b003I, b009U, b003A, b007C, Asm01, Gsm01, 5MSfC, Usm04, 5MRdT, Csm15, Csm16, rCsm14, Csm17 and Tsm18.
2215. The phosphoramidite or compound of any one of Embodiments 2137-2214, wherein R^{NS} is bonded to phosphorus through its 3′-O—.
2216. The phosphoramidite or compound of any one of Embodiments 2140-2215, wherein the purity of the phosphoramidite is at least 85%, 90%, 95%, 96%, 97%, 98%, or 99%.
2217. A method for preparing an oligonucleotide or composition, comprising coupling a —OH group of an oligonucleotide or a nucleoside with a phosphoramidite or compound of any one of Embodiments 2134-2216.
2218. A method for preparing an oligonucleotide or composition, comprising coupling a 5′—OH of an oligonucleotide or a nucleoside with a phosphoramidite or compound of any one of Embodiments 2134-2216.
2219. A method for preparing an oligonucleotide or composition, comprising removing a chiral auxiliary moiety from an oligonucleotide of any one of Embodiments 1994-2133.
2220. The method of any one of Embodiments 2217-2219, wherein the oligonucleotide, or an oligonucleotide in the composition, comprises a sugar comprising 2′-OH.
2221. The method of any one of Embodiments 2217-2220, wherein the oligonucleotide, or an oligonucleotide in the composition, comprises a sugar comprising 2′-OH, wherein the sugar is bonded to a chirally controlled internucleotidic linkage.
2222. The oligonucleotide, composition or method of any one of the preceding Embodiments, wherein each heteroatom is independently selected from nitrogen, oxygen, silicon, phosphorus and sulfur.
2223. The oligonucleotide, composition or method of any one of the preceding Embodiments, wherein each nucleobase independently comprises an optionally substituted ring having at least one nitrogen.
2224. An oligonucleotide or composition of any one of the preceding Embodiments, for editing a target adenosine.
2225. An oligonucleotide or composition of any one of the preceding Embodiments, for preventing or treating a condition, disorder or disease.
2226. A method, comprising:
assessing an agent or a composition thereof in a cell, tissue or animal, wherein the cell, tissue or animal is or comprises a cell, tissue or organ associated or of a condition, disorder or disease, and/or comprises a nucleotide sequence associated with a condition, disorder or disease; and
administering to a subject susceptible to or suffering from a condition, disorder or disease an effective amount of an agent or a composition for preventing or treating the condition, disorder or disease.
   2227. A method, comprising:
administering to a subject susceptible to or suffering from a condition, disorder or disease an effective amount of an agent or a composition for preventing or treating the condition, disorder or disease, wherein the agent or composition is assessed in a cell, tissue or animal, wherein the cell, tissue or animal is or comprises a cell, tissue or organ associated or of a condition, disorder or disease, and/or comprises a nucleotide sequence associated with a condition, disorder or disease.
   2228. The method of Embodiment 2226-2227, wherein the subject is a human.
   2229. The method of any one of Embodiments 2226-2228, wherein a condition, disorder or disease is associated with a G to A mutation.
   2230. The method of any one of Embodiments 2226-2228, wherein a condition, disorder or disease is cancer.
   2231. A method for characterizing an oligonucleotide or a composition, comprising:
administering the oligonucleotide or composition to a cell or a population thereof comprising or expressing an ADAR1 polypeptide or a characteristic portion thereof, or a polynucleotide encoding an ADAR1 polypeptide or a characteristic portion thereof.
   2232. The method of any one of Embodiments 2226-2231, wherein a cell is a rodent cell.
   2233. The method of any one of Embodiments 2226-2231, wherein a cell is a rat cell.
   2234. The method of any one of Embodiments 2226-2231, wherein a cell is a mouse cell.
   2235. The method of any one of Embodiments 2226-2234, wherein the genome of the cell comprises a polynucleotide encoding an ADAR1 polypeptide or a characteristic portion thereof.
   2236. A method for characterizing an oligonucleotide or a composition, comprising:
administering the oligonucleotide or composition to a non-human animal or a population thereof comprising or expressing an ADAR1 polypeptide or a characteristic portion thereof, or a polynucleotide encoding an ADAR1 polypeptide or a characteristic portion thereof.
   2237. The method of Embodiment 2236, wherein the animal is a mouse.
   2238. The method of any one of Embodiments 2236-2237, wherein the genome of the animal comprises a polynucleotide encoding an ADAR1 polypeptide or a characteristic portion thereof.
   2239. The method of any one of Embodiments 2236-2237, wherein the germline genome of the animal comprises a polynucleotide encoding an ADAR1 polypeptide or a characteristic portion thereof.
   2240. The method of any one of Embodiments 2226-2239, wherein an ADAR1 polypeptide or a characteristic portion thereof is or comprises one or both of human ADAR1 Z-DNA binding domains.
   2241. The method of any one of Embodiments 2226-2240, wherein an ADAR1 polypeptide or a characteristic portion thereof is or comprises one or more or all of human ADAR1 dsRNA binding domains.
   2242. The method of any one of Embodiments 2226-2241, wherein an ADAR1 polypeptide or a characteristic portion thereof is or comprises human deaminase domain.
   2243. The method of any one of Embodiments 2226-2242, wherein an ADAR1 polypeptide or a characteristic portion thereof is or comprises human ADAR1.
   2244. The method of any one of Embodiments 2226-2243, wherein an ADAR1 polypeptide or a characteristic portion thereof is or comprises human ADAR1 p¹10.
   2245. The method of any one of Embodiments 2226-2243, wherein an ADAR1 polypeptide or a characteristic portion thereof is or comprises human ADAR1 p150.
   2246. The method of any one of Embodiments 2226-2245, wherein activity levels of an oligonucleotide or composition observed from a cell or a cell from an animal, or a population thereof, is more similar to those observed in a comparable human cell or a population thereof compared to those observed in a cell prior to engineering or a cell from an animal prior to engineering, or a population thereof.
   2247. The method of Embodiment 2246, wherein a comparable human cell is of the same type as a cell or a cell from an animal.
   2248. The method of any one of Embodiments 2226-2247, wherein the cell, tissue or animal is or comprises a cell, tissue or organ associated with or of a condition, disorder or disease.
   2249. The method Embodiment 2248, wherein a cell, tissue or organ associated with or of a condition, disorder or disease is or comprises a tumor.
   2250. The method of any one of Embodiments 2226-2249, wherein the cell, tissue or animal comprises a nucleotide sequence associated with a condition, disorder or disease.
   2251. The method of Embodiment 2250, wherein a nucleotide sequence associated with a condition, disorder or disease is homozygous.
   2252. The method of Embodiment 2250, wherein a nucleotide sequence associated with a condition, disorder or disease is heterozygous.
   2253. The method of Embodiment 2250, wherein a nucleotide sequence associated with a condition, disorder or disease is hemizygous.
   2254. The method of any one of Embodiments 2250-2253, wherein a nucleotide sequence associated with a condition, disorder or disease is in a genome.
   2255. The method of any one of Embodiments 2250-2254, wherein a nucleotide sequence associated with a condition, disorder or disease is in a genome of some but not all cells.
   2256. The method of any one of Embodiments 2250-2255, wherein a nucleotide sequence associated with a condition, disorder or disease is in a germline genome.
   2257. The method of any one of Embodiments 2250-2256, wherein a nucleotide sequence associated with a condition, disorder or disease is a mutation.
   2258. The method of any one of Embodiments 2250-2257, wherein a nucleotide sequence associated with a condition, disorder or disease is a G to A mutation.
   2259. A method for modifying a target adenosine in a target nucleic acid, comprising contacting the target nucleic acid with an oligonucleotide or composition of any one of the preceding Embodiments.
   2260. A method for deaminating a target adenosine in a target nucleic acid, comprising contacting the target nucleic acid with an oligonucleotide or composition of any one of the preceding Embodiments.
   2261. A method for producing, or restoring or increasing level of a particular nucleic acid or a product thereof, comprising contacting a target nucleic acid with an oligonucleotide or composition of any one of the preceding Embodiments, wherein the target nucleic acid comprises a target adenosine, and the particular nucleic acid differs from the target nucleic acid in that the particular nucleic acid has an I or G instead of the target adenosine.
   2262. A method for reducing level of a target nucleic acid or a product thereof, comprising contacting a target nucleic acid with an oligonucleotide or composition of any one of the preceding Embodiments, wherein the target nucleic acid comprises a target adenosine.
   2263. The method of Embodiment 2261 or 2262, wherein the product is a protein.
   2264. The method of Embodiment 2261 or 2262, wherein the product is a mRNA.
   2265. The method of any one of Embodiments 2259-2264, wherein the base sequence the oligonucleotide or oligonucleotides in the oligonucleotide composition is substantially complementary to that of the target nucleic acid.
   2266. The method of any one of Embodiments 2259-2265, wherein the target nucleic acid is in a sample.
   2267. A method, comprising:
contacting an oligonucleotide or composition of any one of the preceding Embodiments with a sample comprising a target nucleic acid and an adenosine deaminase, wherein:
the base sequence of the oligonucleotide or oligonucleotides in the oligonucleotide composition is substantially complementary to that of the target nucleic acid; and the target nucleic acid comprises a target adenosine;
wherein the target adenosine is modified.
   2268. A method, comprising
1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise more sugars with 2′-F modification, more sugars with 2′-OR modification wherein R is not —H, and/or more chiral internucleotidic linkages than oligonucleotides of the reference plurality; and
the first oligonucleotide composition provides a higher level of modification compared to oligonucleotides of the reference oligonucleotide composition.
   2269. A method, comprising
obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise more sugars with 2′-F modification, more sugars with 2′-OR modification wherein R is not —H, and/or more chiral internucleotidic linkages than oligonucleotides of the reference plurality.
   2270. A method, comprising
1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise more sugars with 2′-F modification, more sugars with 2′-OR modification wherein R is not —H, and/or more chirally controlled chiral internucleotidic linkages than oligonucleotides of the reference plurality; and
the first oligonucleotide composition provides a higher level of modification compared to oligonucleotides of the reference oligonucleotide composition.
   2271. A method, comprising
obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise more sugars with 2′-F modification, more sugars with 2′-OR modification wherein R is not —H, and/or more chirally controlled chiral internucleotidic linkages than oligonucleotides of the reference plurality.
   2272. A method, comprising
1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise one or more chirally controlled chiral internucleotidic linkages; and
oligonucleotides of the reference plurality comprise no chirally controlled chiral internucleotidic linkages (a reference oligonucleotide composition is a “stereorandom composition); and
the first oligonucleotide composition provides a higher level of modification compared to oligonucleotides of the reference oligonucleotide composition.
   2273. A method, comprising
obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise one or more chirally controlled chiral internucleotidic linkages; and
oligonucleotides of the reference plurality comprise no chirally controlled chiral internucleotidic linkages (a reference oligonucleotide composition is a “stereorandom composition).
   2274. The method of any one of Embodiments 2268-2273, wherein a first oligonucleotide composition is an oligonucleotide composition of any one of the preceding Embodiments.
   2275. The method of any one of Embodiments 2268-2274, wherein the reference oligonucleotide composition is a reference oligonucleotide composition of any one of Embodiments 2095-2104.
   2276. The method of any one of Embodiments 2259-2275, wherein the deaminase is an ADAR enzyme.
   2277. The method of any one of Embodiments 2259-2275, wherein the deaminase is ADAR1.
   2278. The method of any one of Embodiments 2259-2275, wherein the deaminase is ADAR2.
   2279. The method of any one of Embodiments 2259-2278, wherein the target nucleic acid is or comprise RNA.
   2280. The method of any one of Embodiments 2259-2279, wherein a sample is a cell.
   2281. The method of any one of Embodiments 2259-2280, wherein the target nucleic acid is more associated with a condition, disorder or disease, or decrease of a desired property or function, or increase of an undesired property or function, compared to a nucleic acid which differs from the target nucleic acid in that it has an I or G at the position of the target adenosine instead of the target adenosine.
   2282. The method of any one of Embodiments 2259-2280, wherein the target adenosine is a G to A mutation.
   2283. A method for preventing or treating a condition, disorder or disease, comprising administering or delivering to a subject susceptible thereto or suffering therefrom an effective amount of an oligonucleotide or composition of any one of the preceding Embodiments.
   2284. A method for preventing or treating a condition, disorder or disease amenable to a G to A mutation, comprising administering or delivering to a subject susceptible thereto or suffering therefrom an effective amount of an oligonucleotide or composition of any one of the preceding Embodiments.
   2285. A method for preventing or treating a condition, disorder or disease amenable to a G to A mutation, comprising administering to a subject susceptible thereto or suffering therefrom an effective amount of an oligonucleotide or composition of any one of the preceding Embodiments.
   2286. A method for preventing or treating a condition, disorder or disease associated with a G to A mutation, comprising administering or delivering to a subject susceptible thereto or suffering therefrom an effective amount of an oligonucleotide or composition of any one of the preceding Embodiments.
   2287. A method for preventing or treating a condition, disorder or disease associated with a G to A mutation, comprising administering to a subject susceptible thereto or suffering therefrom an effective amount of an oligonucleotide or composition of any one of the preceding Embodiments.
   2288. The method of any one of Embodiments 2283-2287, wherein the base sequence of the oligonucleotide or oligonucleotides in the oligonucleotide composition is substantially complementary to that of the target nucleic acid comprising a target adenosine that is the mutation.
   2289. The method of any one of Embodiments 2287-2288, wherein the condition, disorder or disease is amenable to an A to G or A to I modification.
   2290. The method of any one of Embodiments 2283-2289, wherein cells associated with the condition, disorder or disease comprise or express an ADAR protein.
   2291. The method of any one of Embodiments 2283-2289, wherein cells associated with the condition, disorder or disease comprise or express ADAR1.
   2292. The method of any one of Embodiments 2283-2289, wherein cells associated with the condition, disorder or disease comprise or express ADAR2.
   2293. The method of any one of Embodiments 2283-2292, wherein the subject is a human subject.
   2294. The method of any one of Embodiments 2259-2293, comprising converting a target adenosine to I.
   2295. The method of any one of Embodiments 2259-2294, wherein two or more different adenosine are targeted and edited.
   2296. The method of any one of Embodiments 2259-2294, wherein two or more different transcripts are targeted and edited.
   2297. The method of any one of Embodiments 2259-2294, wherein transcripts from two or more different polynucleotides are targeted and edited.
   2298. The method of any one of Embodiments 2259-2294, wherein transcripts from two or more genes are targeted and edited.
   2299. The method of any one of Embodiments 2295-2298, comprising administering two or more oligonucleotides, each of which independently targets a different target, and each of which is independently an oligonucleotide of any one of Embodiments 1-1992 or a salt thereof.
   2300. The method of any one of Embodiments 2295-2298, comprising administering two or more oligonucleotide compositions, each of which independently targets at least one different target, and each of which is independently a composition of any one of Embodiments 1993-2107.
   2301. The method of any one of Embodiments 2295-2300, comprising administering a composition of any one of Embodiments 2018-2107.
   2302. The method of any one of Embodiments 2295-2301, wherein two or more oligonucleotides or compositions are administered concurrently.
   2303. The method of any one of Embodiments 2295-2302, wherein two or more oligonucleotides or compositions are administered concurrently in a single composition.
   2304. The method of any one of Embodiments 2295-2302, wherein two or more oligonucleotides or compositions are administered as separated compositions.
   2305. The method of any one of Embodiments 2295-2301, wherein one or more oligonucleotides or compositions are administered prior or subsequently to one or more other oligonucleotides or compositions.
   2306. Use of an oligonucleotide or composition of any one of the preceding Embodiments for alter mRNA splicing, wherein a target adenosine of an mRNA is edited.
   2307. The use of Embodiment 2308, wherein an exon is skipped, or an exon is included, or frame is restored.
   2308. Use of an oligonucleotide or composition of any one of the preceding Embodiments for alter mRNA splicing, wherein a target adenosine of an mRNA is edited.
   2309. The use of Embodiment 2308, wherein levels of a RNA and/or a polypeptide encoded thereby is reduced.
   2310. Use of an oligonucleotide or composition of any one of the preceding Embodiments for silencing protein expression, wherein a target adenosine of an mRNA encoding the protein is edited.
   2311. The use of Embodiment 2310, wherein expression, level and/or activity of a protein is increased or restored.
   2312. Use of an oligonucleotide or composition of any one of the preceding Embodiments for fixing nonsense mutation, wherein a target adenosine of an RNA is edited so that the nonsense mutation is fixed.
   2313. The use of Embodiment 2312, wherein expression, level and/or activity of a protein is increased or restored.
   2314. Use of an oligonucleotide or composition of any one of the preceding Embodiments for fixing missense mutation, wherein a target adenosine of an RNA is edited so that the missense mutation is fixed.
   2315. The use of Embodiment 2314, wherein expression, level and/or activity of a protein is increased or restored.
   2316. Use of an oligonucleotide or composition of any one of the preceding Embodiments for editing a target adenosine in a codon.
   2317. The use of Embodiment 2316, wherein sequence, expression, level and/or activity of a protein is altered.
   2318. Use of an oligonucleotide or composition of any one of the preceding Embodiments for editing a target adenosine in an upstream ORF.
   2319. The use of Embodiment 2318, wherein expression, level and/or activity of a protein is increased.

2320. A method for modulating protein-protein interaction in a system wherein a protein is translated from its encoding RNA, comprising contacting the encoding RNA with an oligonucleotide or composition of any one of the preceding Embodiments, wherein an adenosine in the encoding RNA is edited, wherein a protein is translated from the encoded mRNA (“the edited protein”), wherein the edited protein differs from the unedited protein at an amino acid residue involving in the protein-protein interaction.

2321. A method for modulating an interaction between a protein and its partner protein in a system, comprising administering to the system of any one of the preceding Embodiments, wherein the oligonucleotide or composition is capable of editing an adenosine in a nucleic acid encoding the protein or its partner protein, and an edited nucleic acid encodes a protein that is different from the protein encoded by the unedited nucleic acid at at least one amino acid residue involved in the interaction between the protein and its partner protein.
2322. The method of any one of Embodiments 2320-2321, wherein the edited adenosine is in a codon encoding an amino acid residue involved in the interaction between the protein and its partner protein.
2323. The method of any one of Embodiment 2322, wherein the edited adenosine is in a codon encoding an amino acid residue involved in the interaction between the protein and its partner, and the editing changed the amino acid to a different amino acid.
2324. The method of any one of Embodiments 2320-2323, wherein the protein-protein interaction is reduced or disrupted.
2325. The method of any one of Embodiments 2320-2324, wherein the protein is a transcription factor.
2326. The method of any one of Embodiments 2320-2325, wherein level of the protein is increased.
2327. The method of any one of Embodiments 2320-2326, wherein expression of one or more nucleic acids regulated by the protein is modulated.
2328. The method of any one of Embodiments 2320-2327, wherein expression of one or more nucleic acids regulated by the protein is increased.
2329. The method of any one of Embodiments 2320-2328, wherein the system is or comprises a cell.
2330. The method of any one of Embodiments 2320-2329, wherein the system is or comprises a tissue.
2331. The method of any one of Embodiments 2320-2330, wherein the system is or comprises an organ.
2332. The method of any one of Embodiments 2320-2331, wherein the system is or comprises an organism.
2333. A method for editing a transcript in an immune cell, comprising administering to an immune cell an effective amount of an oligonucleotide or composition of any one of the preceding Embodiments.
2334. The method of Embodiment 2333, wherein an immune cell is a PBMC.
2335. The method of Embodiment 2333, wherein an immune cell is a CD4+ cell.
2336. The method of Embodiment 2333, wherein an immune cell is a CD8+ cell.
2337. The method of Embodiment 2333, wherein an immune cell is a CD14+ cell.
2338. The method of Embodiment 2333, wherein an immune cell is a CD19+ cell.
2339. The method of Embodiment 2333, wherein an immune cell is a NK cell.
2340. The method of Embodiment 2333, wherein an immune cell is a Treg cell.
2341. The method of any one of Embodiments 2333-2340, wherein the cell is activated.
2342. The method of any one of Embodiments 2333-2340, wherein the cell is non-activated.
2343. The method of any one of Embodiments 2333-2342, wherein the oligonucleotide or composition targets and edits FAS, BID, CTLA4, PDCD1, CBLB, PTPN6, TRAC, or TRBC.
2344. A method for improving editing levels of an oligonucleotide, comprising incorporating a structural element recited in any one of the preceding Embodiments.
2345. The method of any one of the preceding Embodiments, wherein a target adenosine opposite to a nucleoside in a first domain is edited.
2346. The method of any one of the preceding Embodiments, wherein a target adenosine opposite to No in a first domain is edited.
2347. The method of any one of the preceding Embodiments, wherein a target adenosine opposite to a nucleoside in a second domain is edited.
2348. The method of any one of the preceding Embodiments, wherein a target adenosine opposite to No in a second domain is edited.
2349. A compound, oligonucleotide, composition, nucleobase, sugar, nucleoside, internucleotidic linkage, or method described in the present disclosure.

### EXEMPLIFICATION

Certain examples of provided technologies (compounds (oligonucleotides, reagents, etc.), compositions, methods (methods of preparation, use, assessment, etc.), etc.) were presented herein. Those skilled in the art appreciate that many technologies, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/046667, WO 2022/099159, PCT/US2023/018742, etc., can be utilized to prepare and/or assess properties and/or activities of provided technologies in accordance with the present disclosure.

### Example 1. Useful Technologies for Assessing Adenosine Editing

Oligonucleotide designs may be assessed using various systems. In some embodiments, cLuc assay as described in, e.g., WO 2021/071858, can be utilized to assess oligonucleotides and composition. In some embodiments, oligonucleotides targeting cLuc (Cypridina) are assessed in 293T cells transfected with plasmids for either human ADAR1 or human ADAR2 and a cLuc luciferase reporter plasmid. In some embodiments, a cLuc reporter plasmid is or comprises (Gaussia)gLuc-p2A-cLuc(W85X) with respect to luciferases. In some embodiments, a cLuc reporter is activated by ADAR mediated A>I editing. In some embodiments, a reporter plasmid and ADAR1 or ADAR2 plasmid are transfected together into HEK293T cells using the Lipofectamine 2000 transfection protocol (Thermo 11668030). After a suitable time period, e.g., 24 hours, the HEK293T cells expressing the reporter and ADAR plasmids are reverse transfected with the appropriate amount of oligonucleotides for each experiment. In some embodiments, cLuc and gLuc activity is measured after 48, 72, and/or 96 hours using the Pierce™ Gaussia Luciferase Glow Assay Kit (Pierce™ 16161) or the Pierce™ Cypridina Luciferase Glow Assay Kit (Pierce™ 16170), respectively.

In some embodiments, oligonucleotides and compositions can be assessed and confirmed to provide editing in various cells, e.g., mouse or human primary hepatocytes, primary human retinal pigment epithelial cells, cell lines, etc. In some embodiments, editing of ACTb is assessed in cells.

In some embodiments, oligonucleotide and compositions are assessed and confirmed to provide editing in subjects. In some embodiments, oligonucleotides and compositions are assessed and confirmed to provide editing in animals, e.g., mice, non-human primates (e.g., cynomoI gus macaques), etc. In some embodiments, animals are transgenic animals, e.g., mice expressing human ADAR1. In some embodiments, animals are model animals comprising target adenosines associated with conditions, disorders or diseases, e.g., in many instances, G to A mutations. In some embodiments, provided technologies can provide efficient editing with or without exogenous ADAR polypeptides. In some embodiments, provided technologies can provide efficient editing without exogenous ADAR1 or ADAR2. In some embodiments, oligonucleotides and compositions are delivered by transfection (e.g., using transfection compositions such as Lipofectamine RNAimax). In some embodiments, oligonucleotides and compositions are delivered by gymnotic free update. Certain useful technologies are described in WO 2021/071858, WO 2022/046667, WO 2022/099159, and/or PCT/US2023/018742.

In some embodiments, provided technologies can provide high levels of selectivity. In some embodiments, about or at least about 80%, 85%, 90%, 91%, 92%, 93%, 94%, 95%, 95%, 96%, 97%, 98%, 99%, 99.5%, or 99.9% observed adenosine editing are at target adenosines. In some embodiments, about or at least about 80%, 85%, 90%, 95%, 96%, 97%, 98%, or 99% observed adenosine editing in coding regions are at target adenosines. In some embodiments, about or at least about 80%, 85%, 90%, 95%, 96%, 97%, 98%, or 99% observed adenosine editing in target nucleic acids (e.g., transcripts of target genes) are at target adenosines. In some embodiments, about or at least about 80%, 85%, 90%, 95%, 96%, 97%, 98%, or 99% observed adenosine editing in coding regions of target nucleic acids (e.g., transcripts of target genes) are at target adenosines. Various technologies, e.g., RNA-Seq, are available to those skilled in the art to assess selectivity; certain such technologies are described herein or in the priority applications or WO 2021/071858, WO 2022/099159, and/or PCT/US2023/018742, the entirety of each of which is independently incorporated herein by reference. In some embodiments, a percentage for a selectivity described herein is at least about 80%. In some embodiments, it is at least about 85%. In some embodiments, it is at least about 90%. In some embodiments, it is at least about 95%. In some embodiments, it is at least about 96%. In some embodiments, it is at least about 97%. In some embodiments, it is at least about 98%. In some embodiments, it is at least about 99%. In some embodiments, it is at least about 99.5%. In some embodiments. it is at least about 99.9%. In some embodiments, it is about 100%. In some embodiments, no off-target editing is observed. In some embodiments, provided technology provides high selectivity in vivo.

In some embodiments, the present disclosure among other things provide oligonucleotides comprising various modifications (e.g., nucleobase modifications, sugar modifications, linkage modifications, etc., and combinations and patterns thereof) that can provide efficient editing.

In some embodiments, provided technologies can provide efficient editing using significantly shorter oligonucleotides compared to various prior reported technologies. In some embodiments, oligonucleotides of various lengths, e.g., 27, 28, 29, 20, 31, 32, or more, nucleosides can provide editing.

In some embodiments, base sequences of oligonucleotides or portions thereof are of sufficient complementarity to those of target nucleic acids so that oligonucleotides can form duplexes under suitable conditions, e.g., in vivo or in vitro editing conditions. In some embodiments, oligonucleotides selectively form duplexes with target nucleic acids over non-target nucleic acids. While certain levels of complementarity to target nucleic acids are preferred or required for various uses including target adenosine editing, full complementarity is generally not required. In some embodiments, there are one or mismatches, bulges, etc. as described herein. In some embodiments, the nucleobase of a nucleoside opposite to target adenosine, N₀, is not complementary to a target adenosine. In some embodiments, hypoxanthine is utilized in place of G particularly if close or next to No. In some embodiments, first domains, first subdomains and/or third subdomains comprise one or more, e.g., 1, 2, 3, 4, or more, mismatches.

In some embodiments, oligonucleotides are provided in chirally controlled oligonucleotide compositions. In some embodiments, as illustrated herein, chirally controlled oligonucleotide compositions provide various desired properties and/or activities. In some embodiments, chirally controlled oligonucleotide compositions provide improved properties and/or activities compared to corresponding stereorandom oligonucleotide compositions (e.g., of oligonucleotides of the same constitution but not chirally controlled at chiral linkage phosphorus).

Among other things, technologies can provide robust editing in the presence of ADAR1 and/or ADAR2, and can provide robust editing in the presence of ADAR1-p110 and/or ADAR1-p150.

### Example 2. Technologies for Preparing Oligonucleotide and Compositions

Various technologies (e.g., phosphoramidites, nucleobases, nucleosides, etc.) for preparing provided technologies (e.g., oligonucleotides, compositions (e.g., oligonucleotide compositions, pharmaceutical compositions, etc.), etc.) can be utilized in accordance with the present disclosure, including, for example, methods and reagents described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the methods and reagents of each of which are incorporated herein by reference. In some embodiments, the present disclosure provides useful technologies for preparing oligonucleotides and compositions thereof.

In some embodiments, useful compounds including those described below or salts thereof. In some embodiments, compounds were prepared utilizing technologies described in the priority applications and WO 2021/071858, WO 2022/099159, and/or PCT/US2023/018742, the entirety of each of which is incorporated herein by reference.

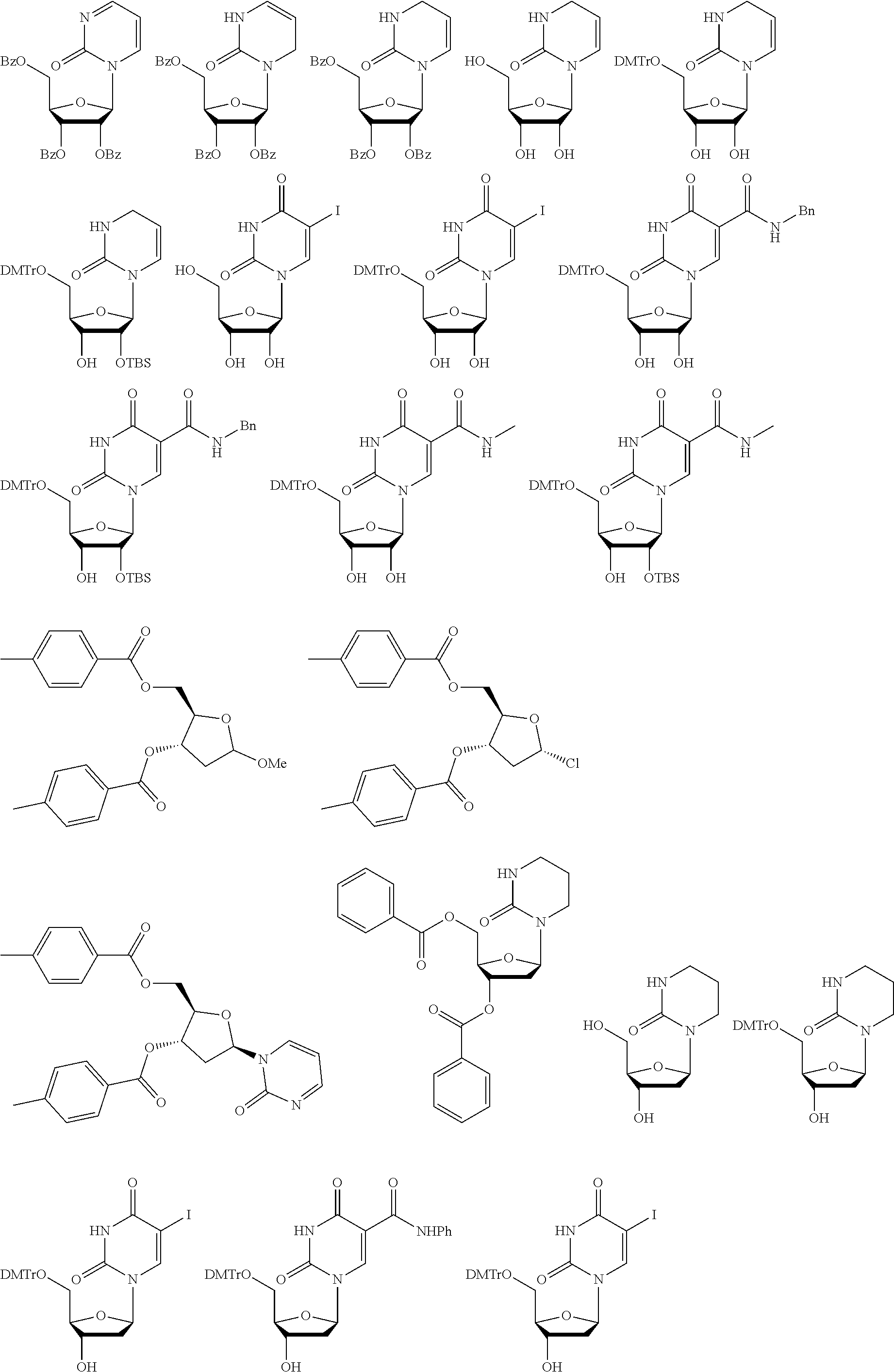

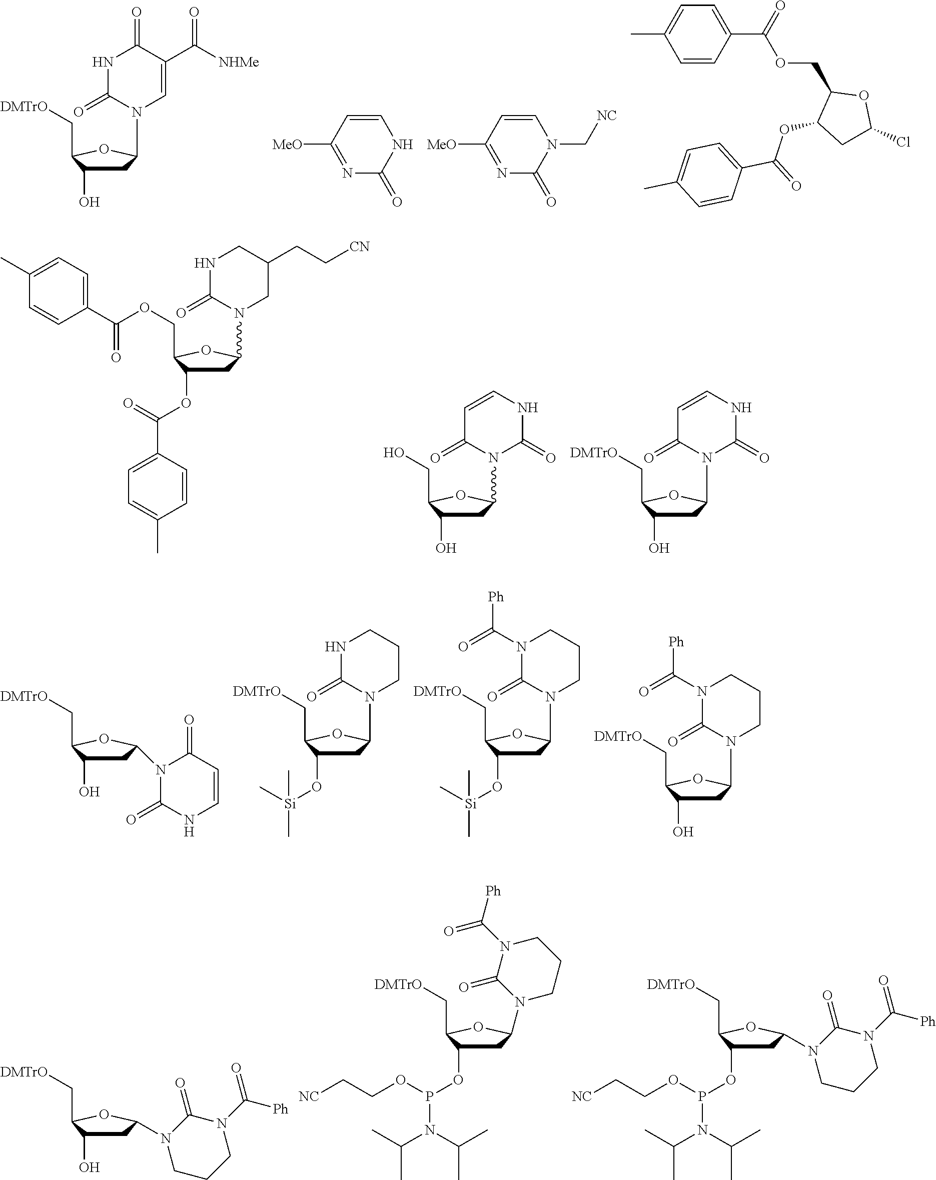

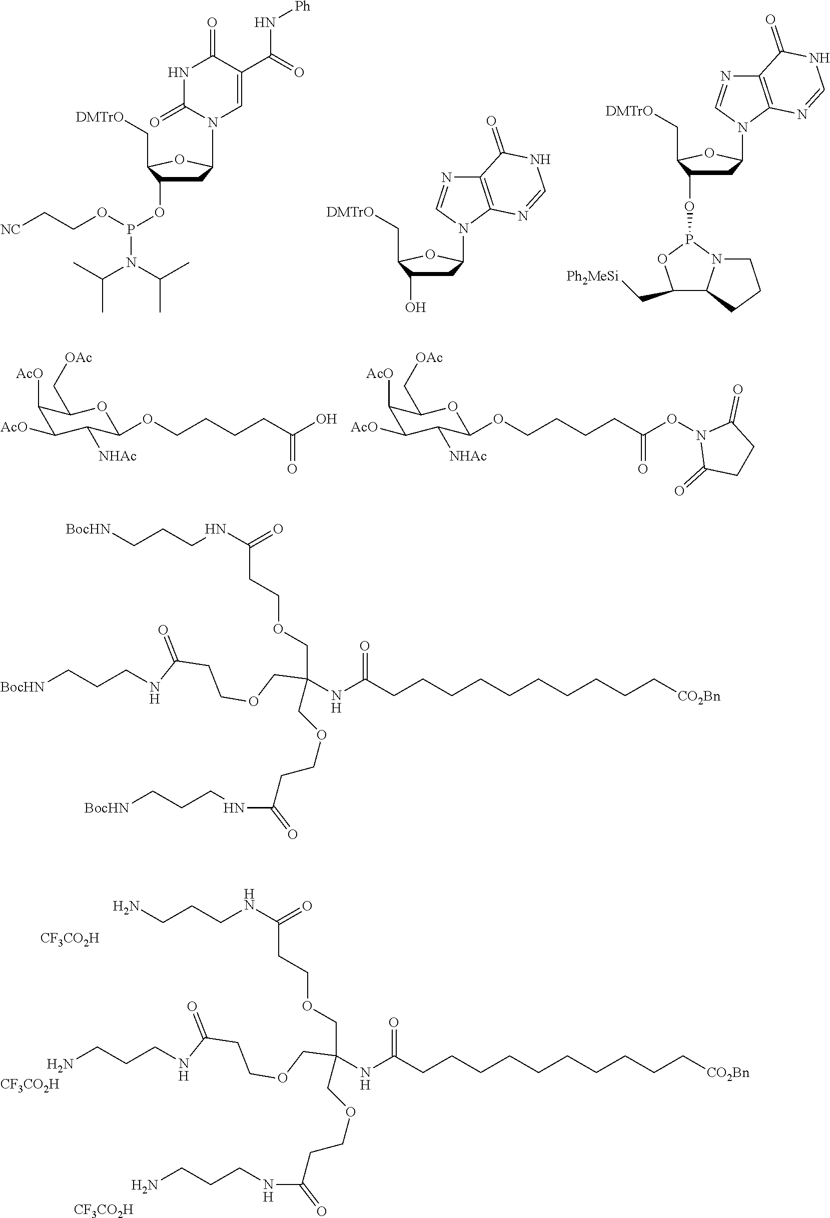

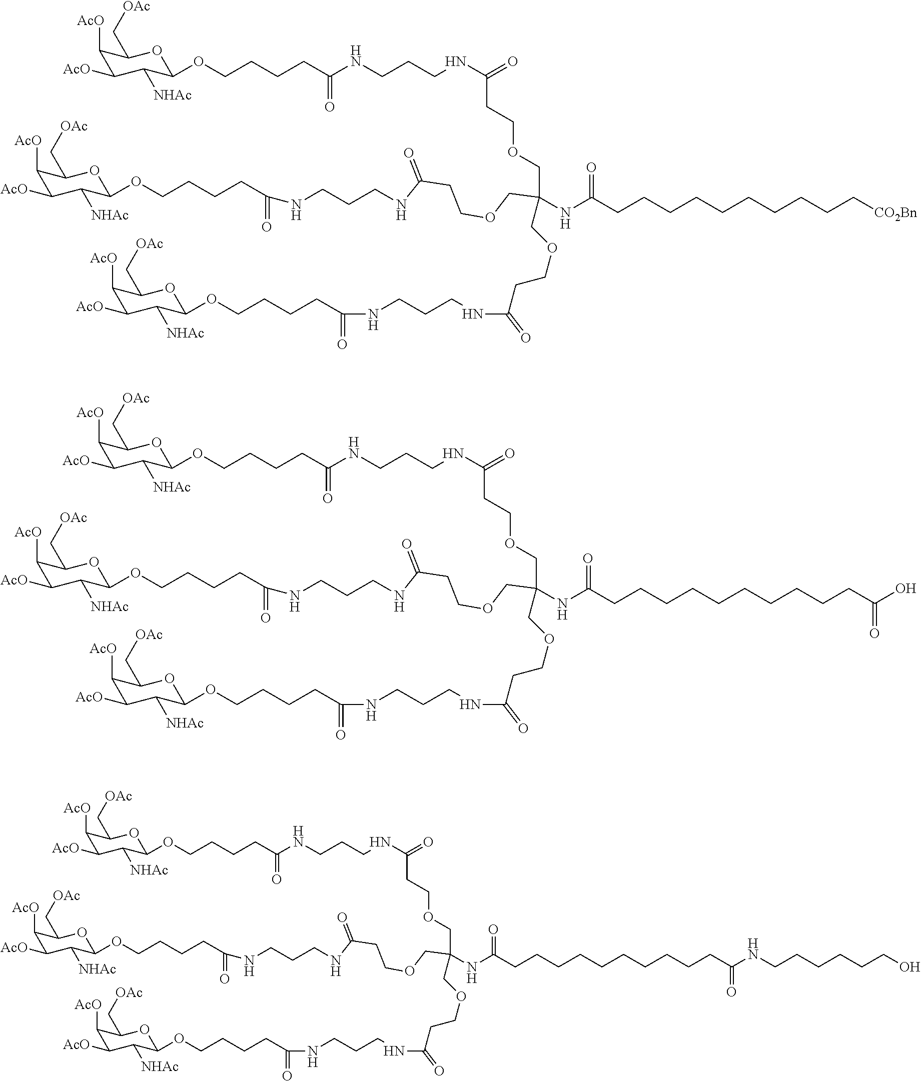

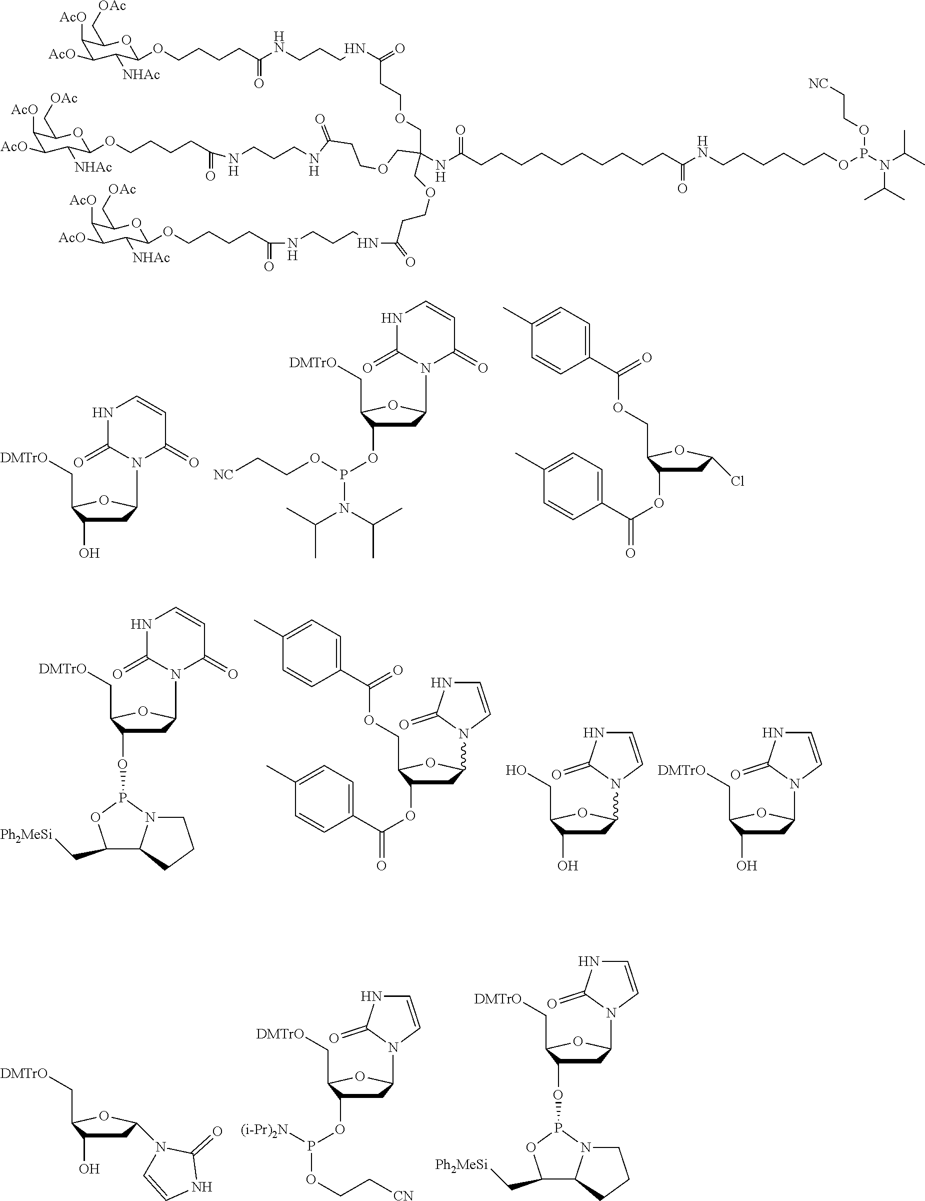

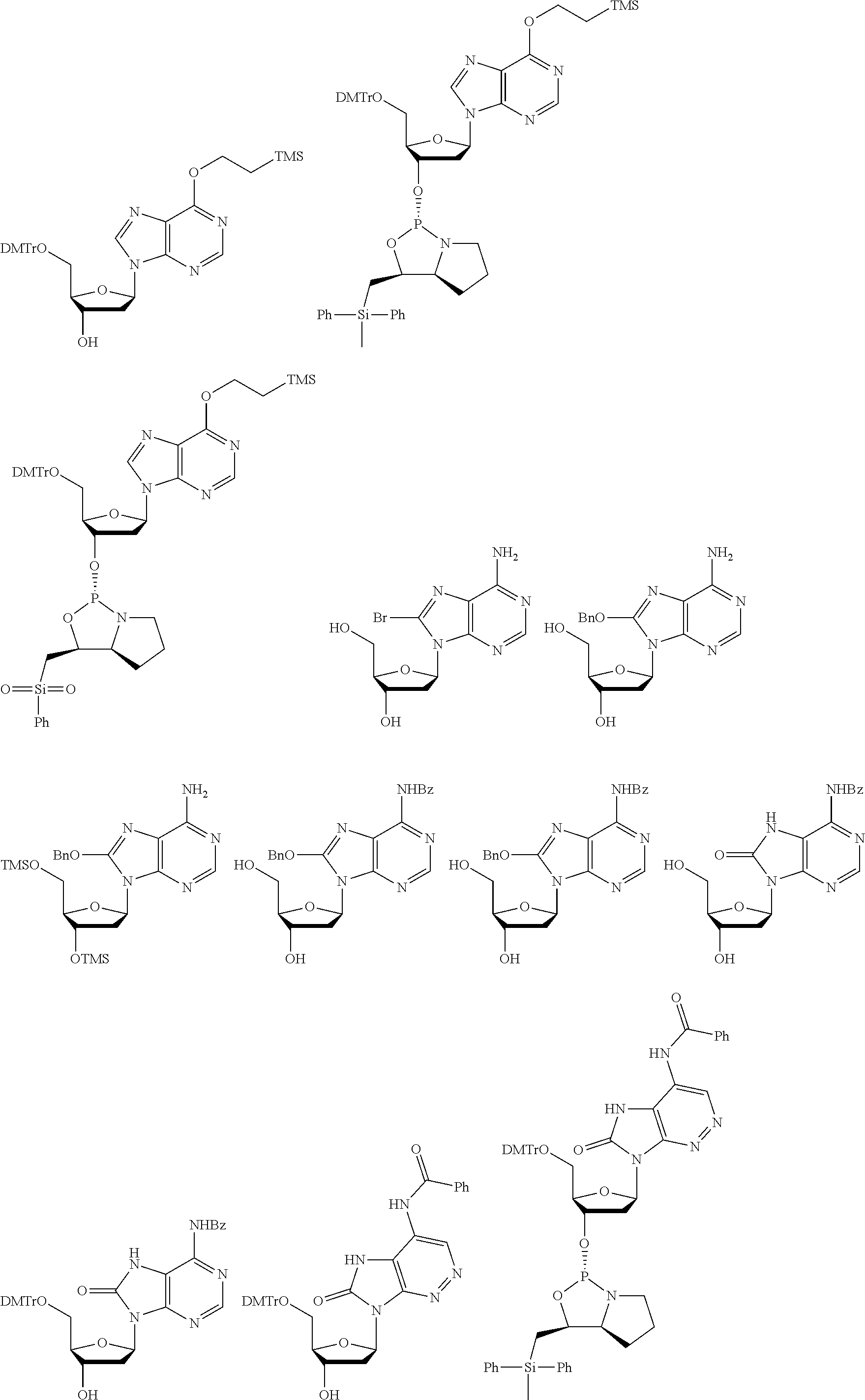

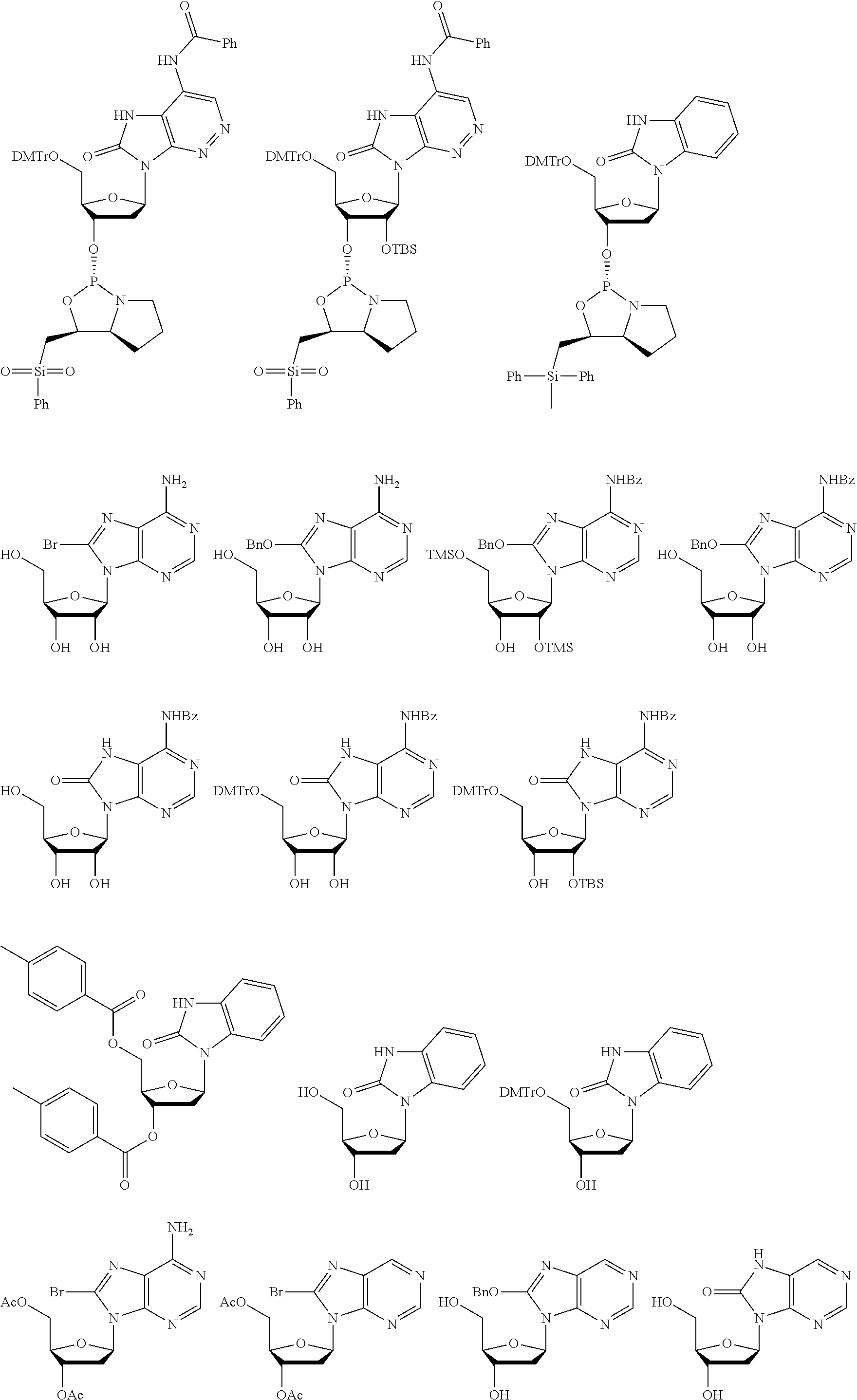

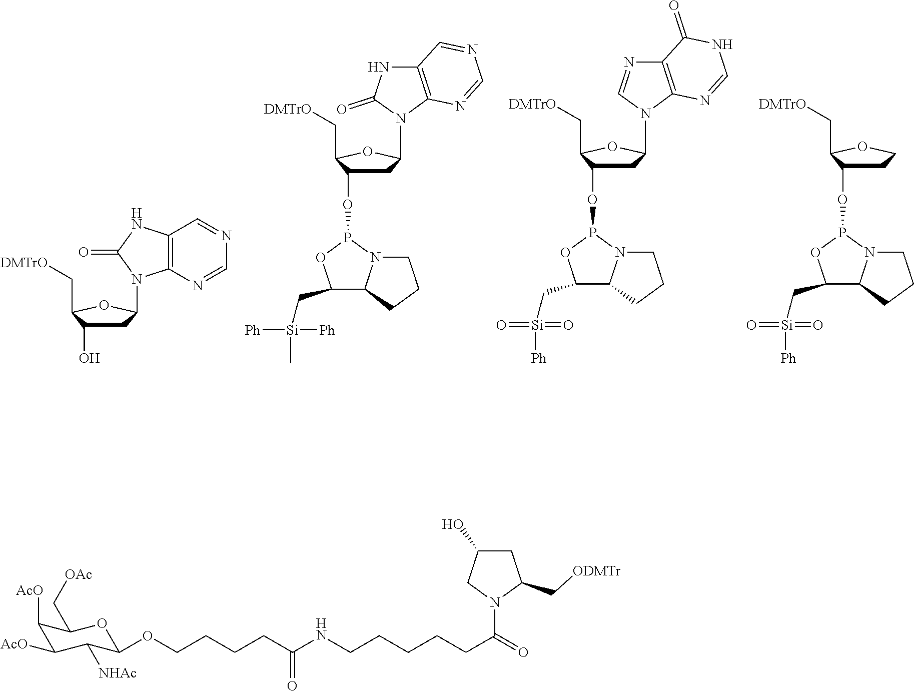

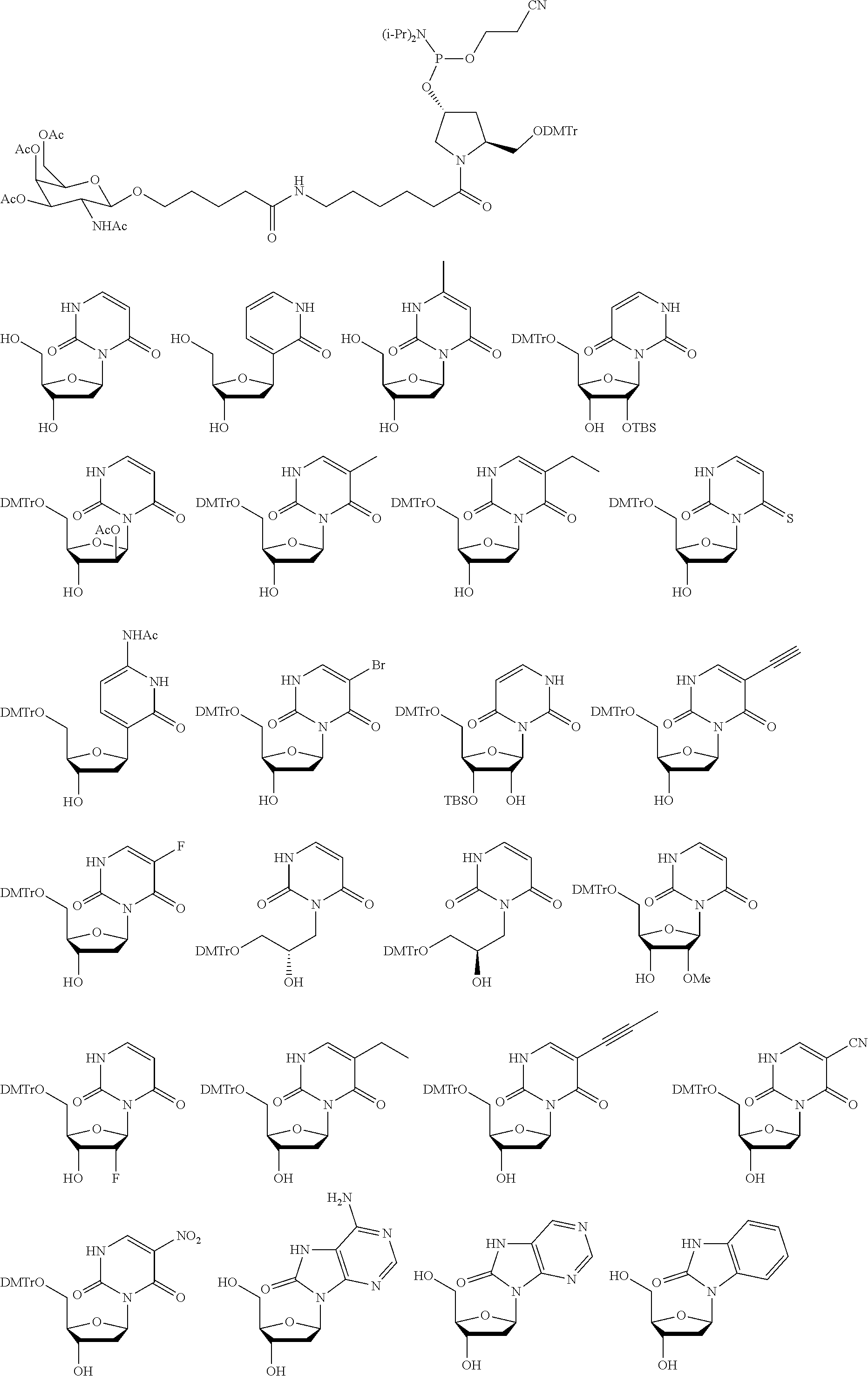

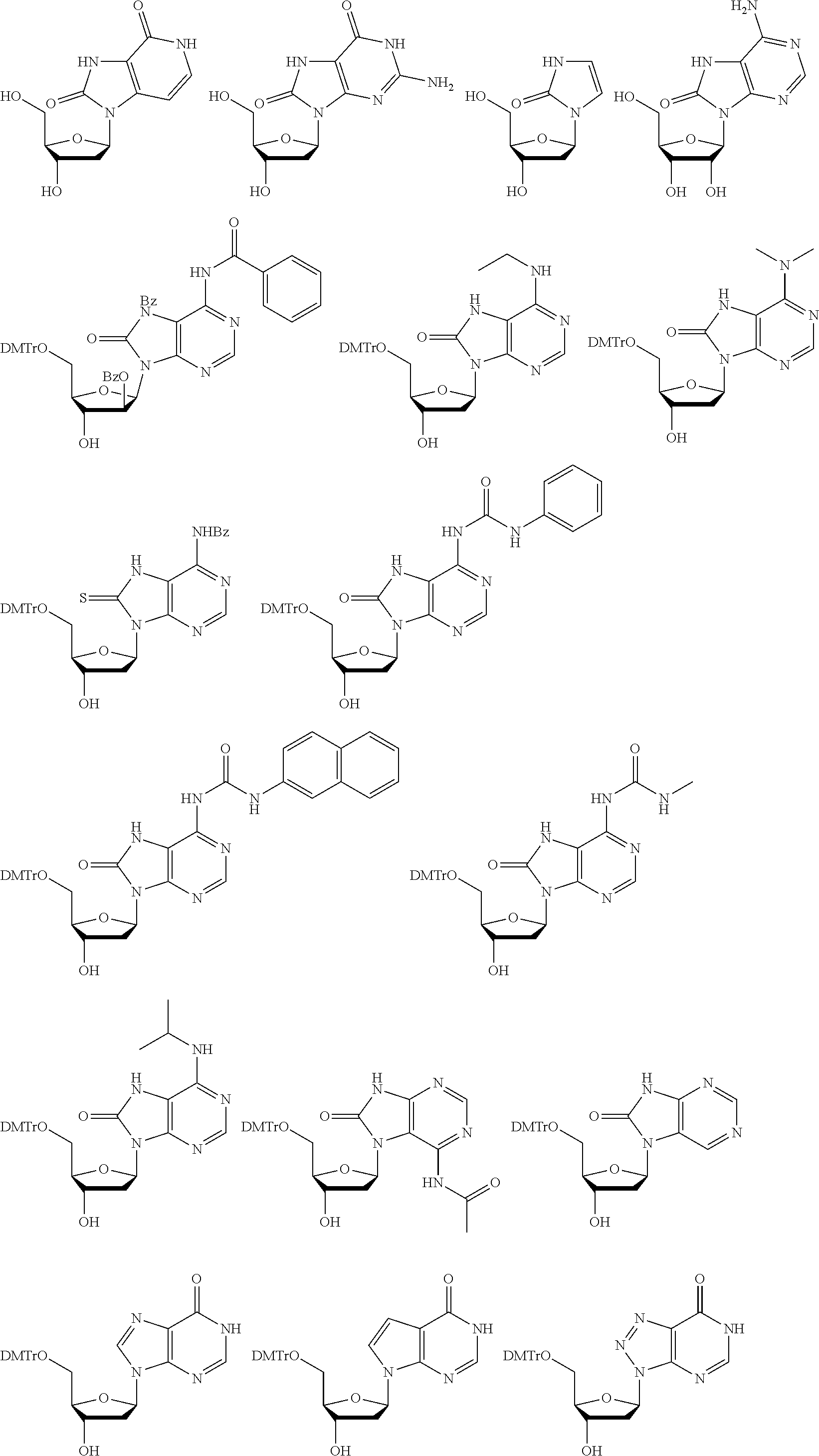

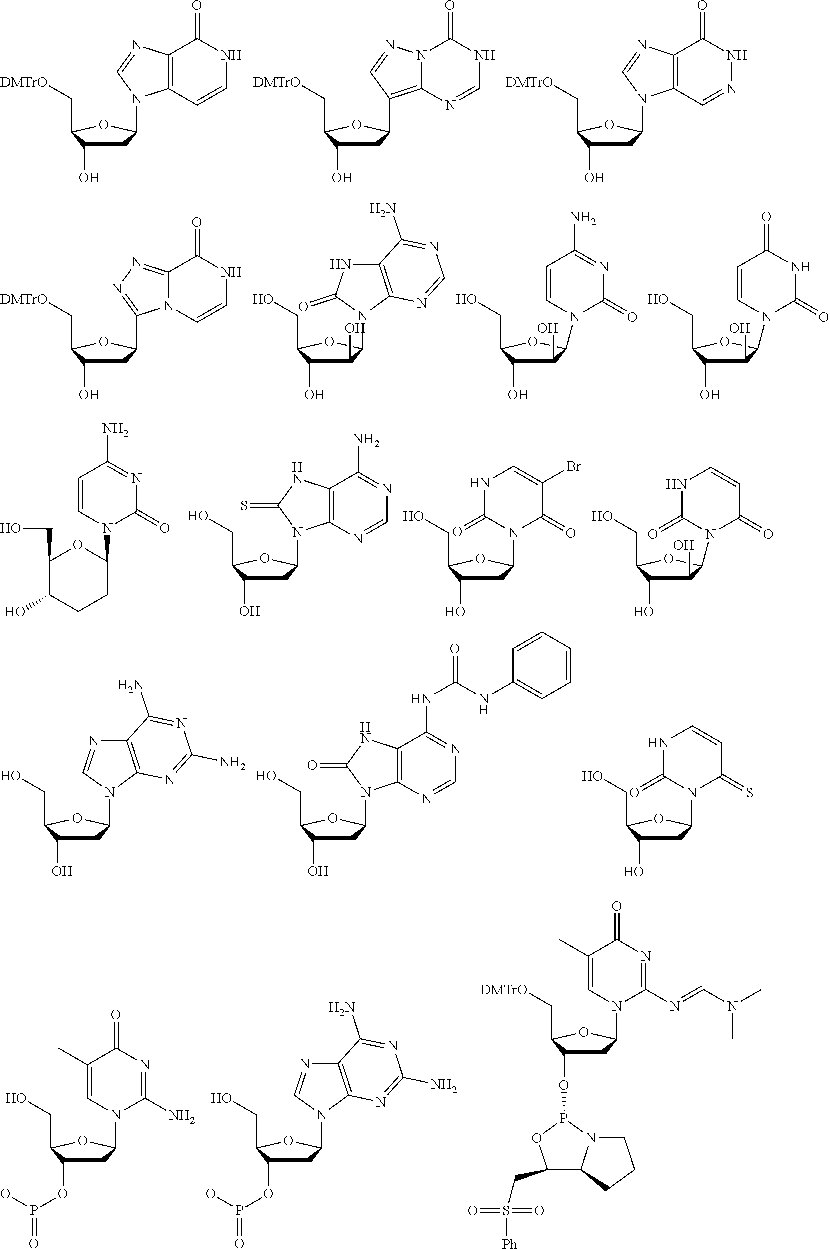

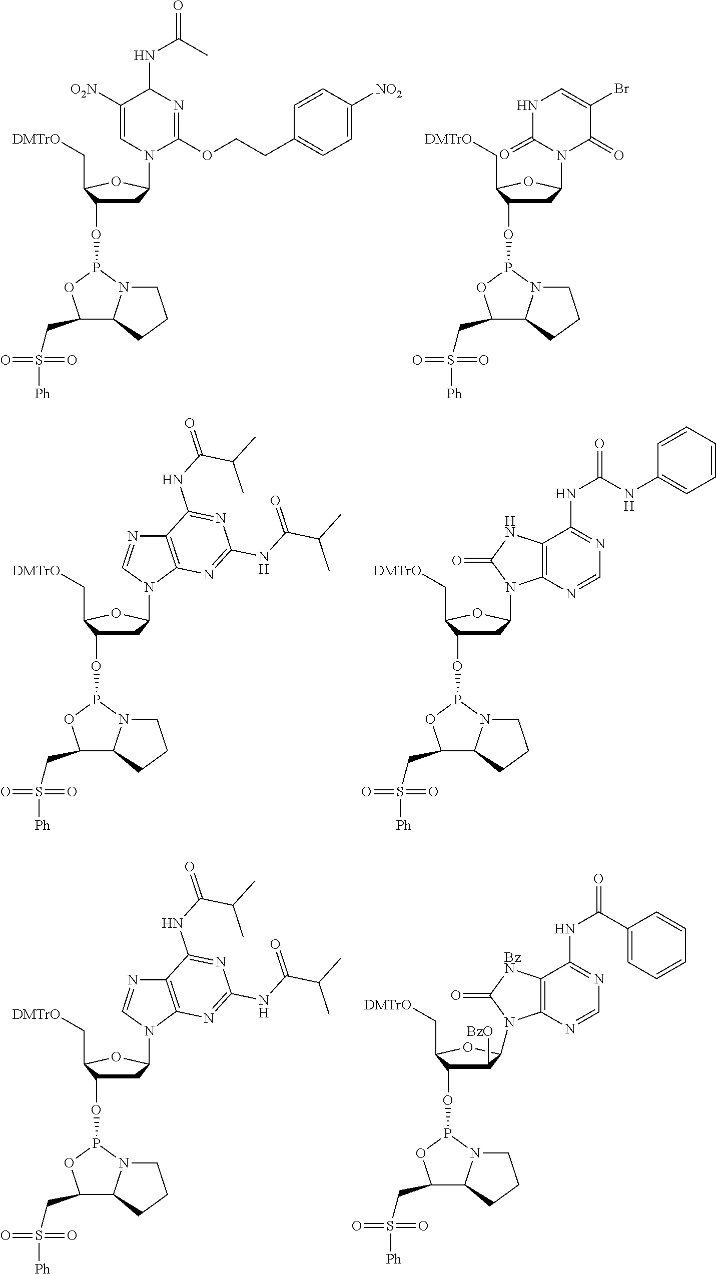

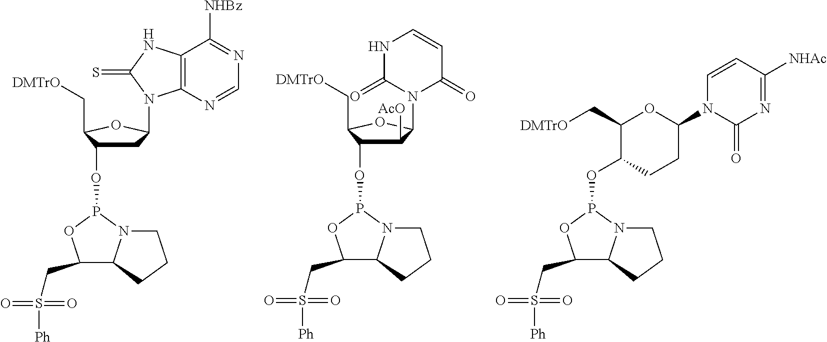

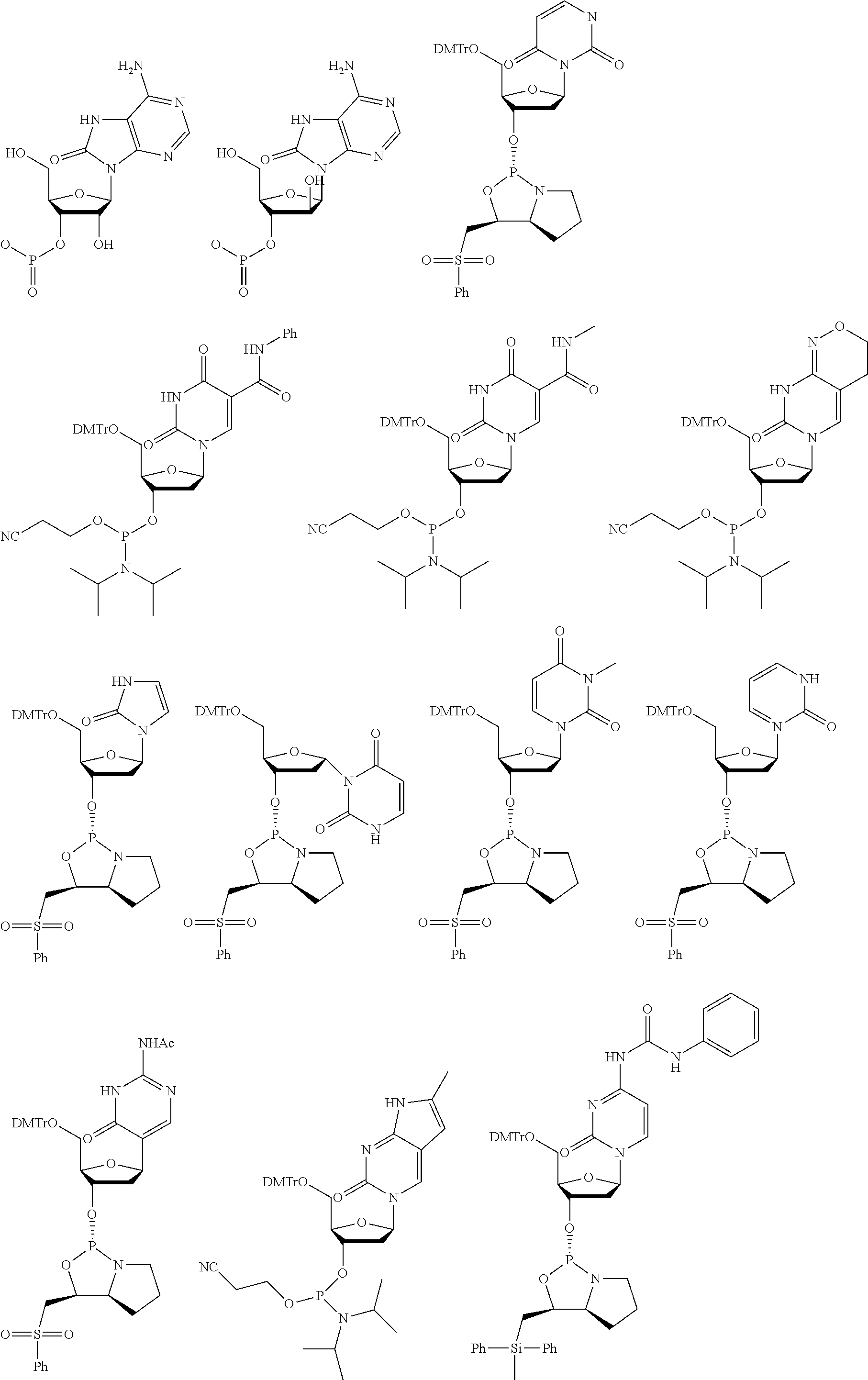

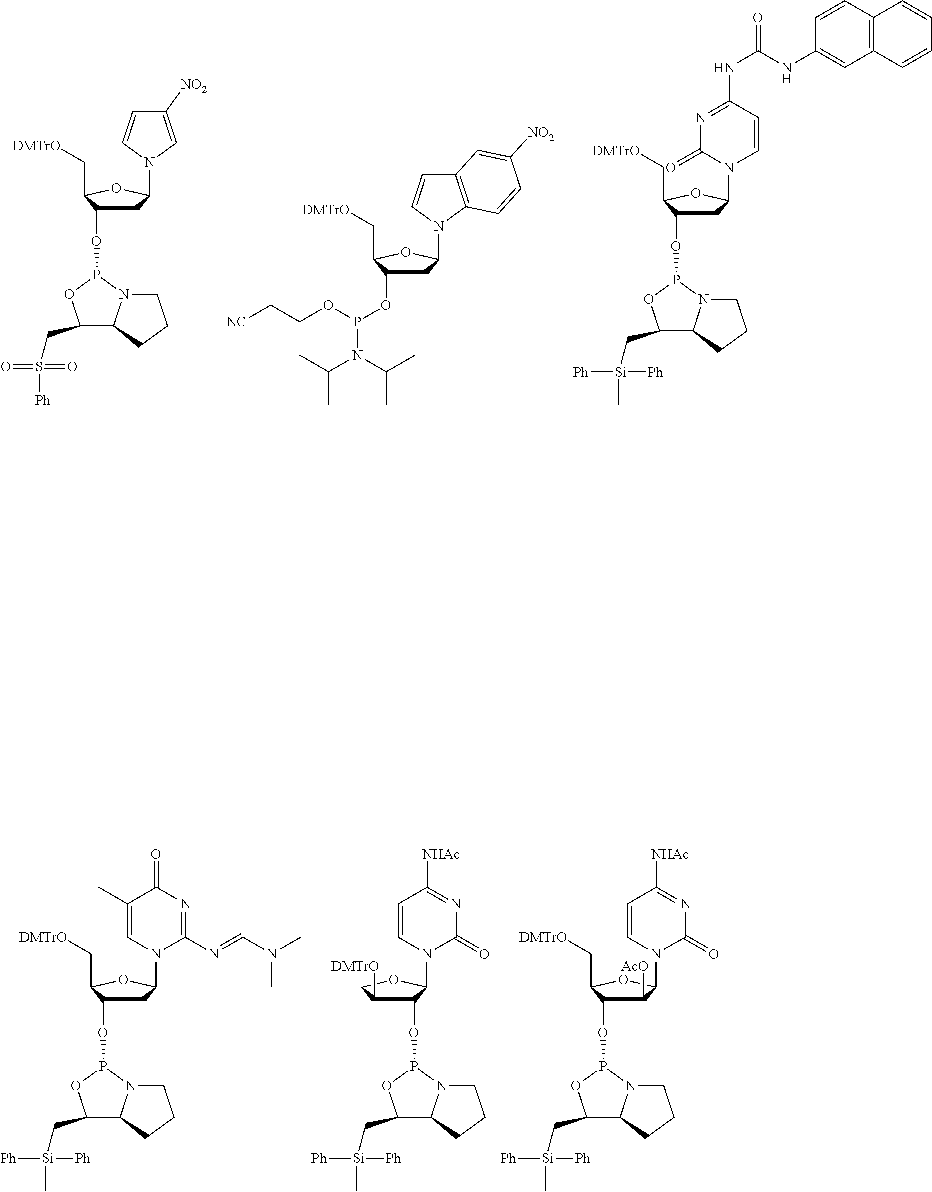

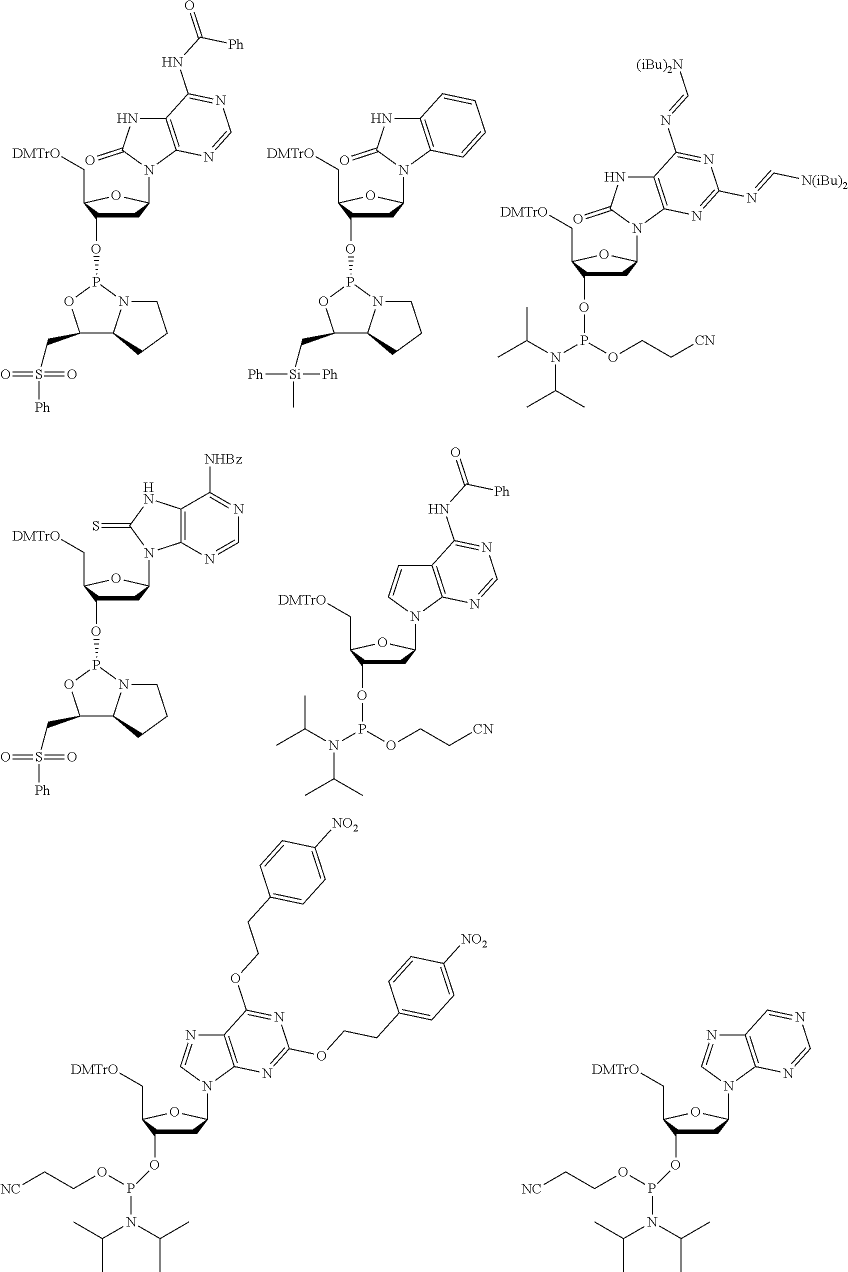

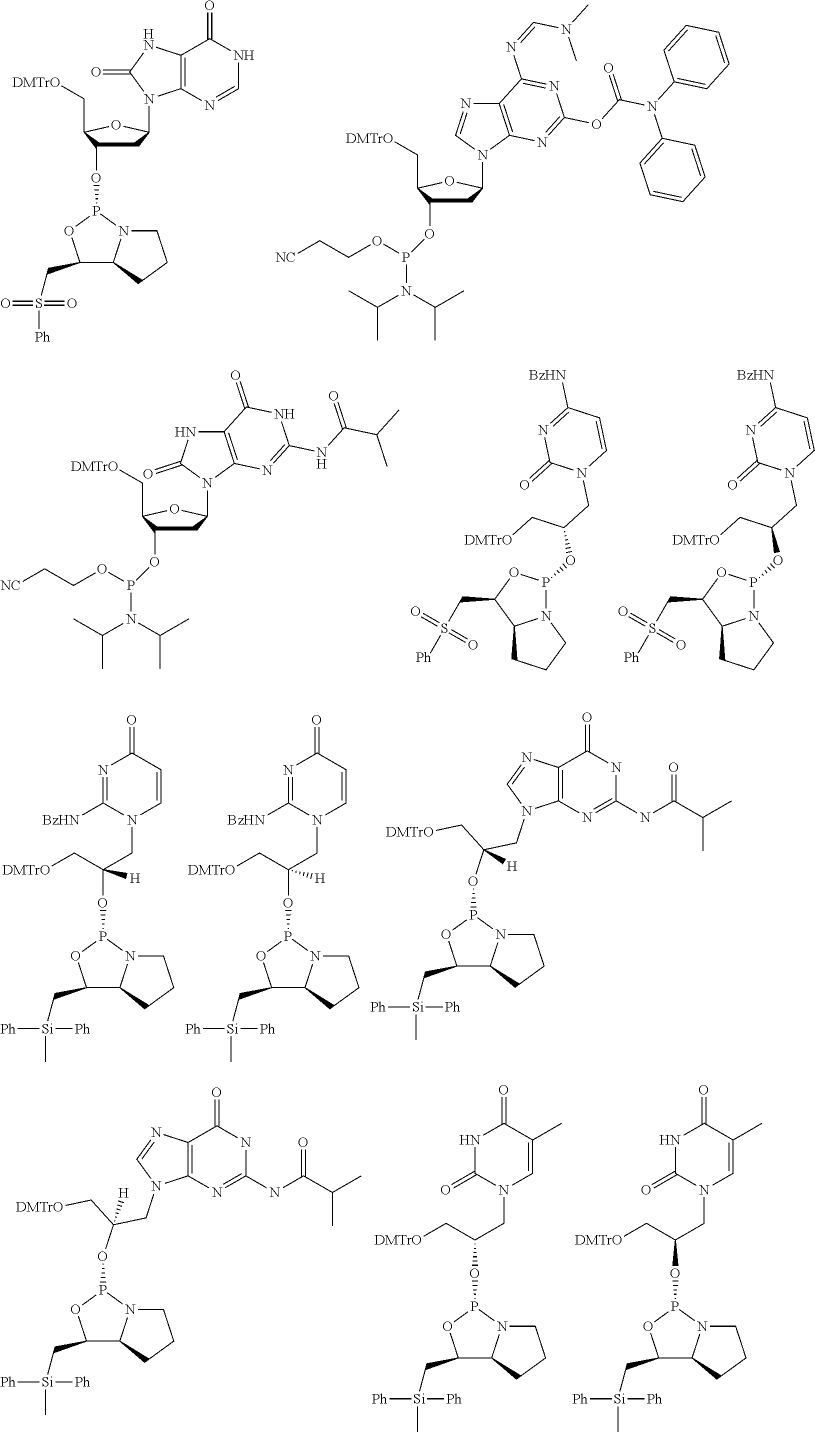

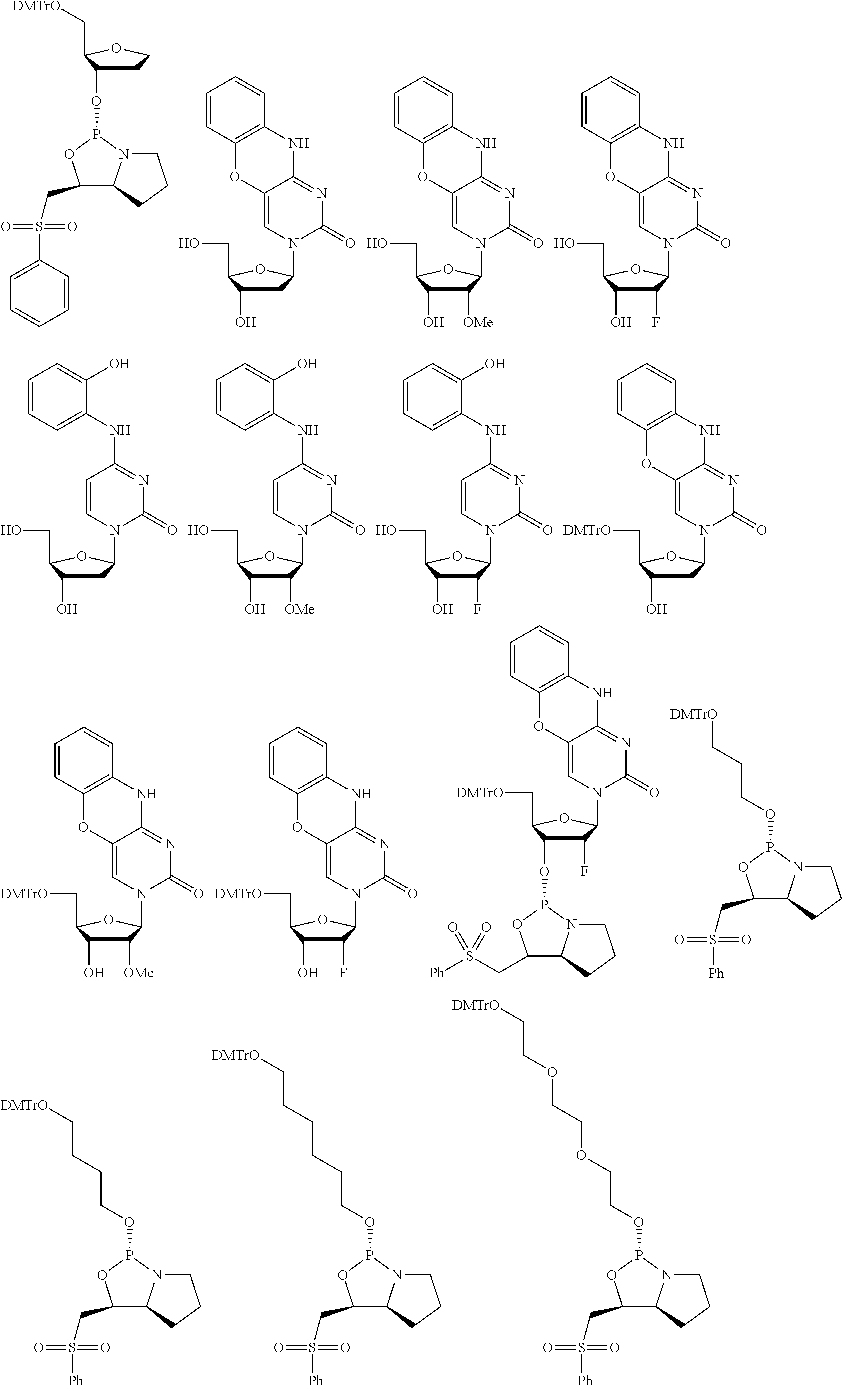

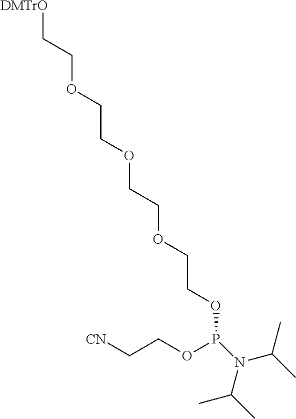

Certain useful technologies for preparing various additional useful compounds are described below as examples.

### Synthesis of (2R, 3S, 4R, 5R)-5-((bis(4-methoxyphenyl)(phenyl)methoxy)methyl)-2-(2, 4-dioxo-3, 4-dihydropyrimidin-1(2H)-yl)-4-hydroxytetrahydrofuran-3-yl acetate (WV-NU-211)

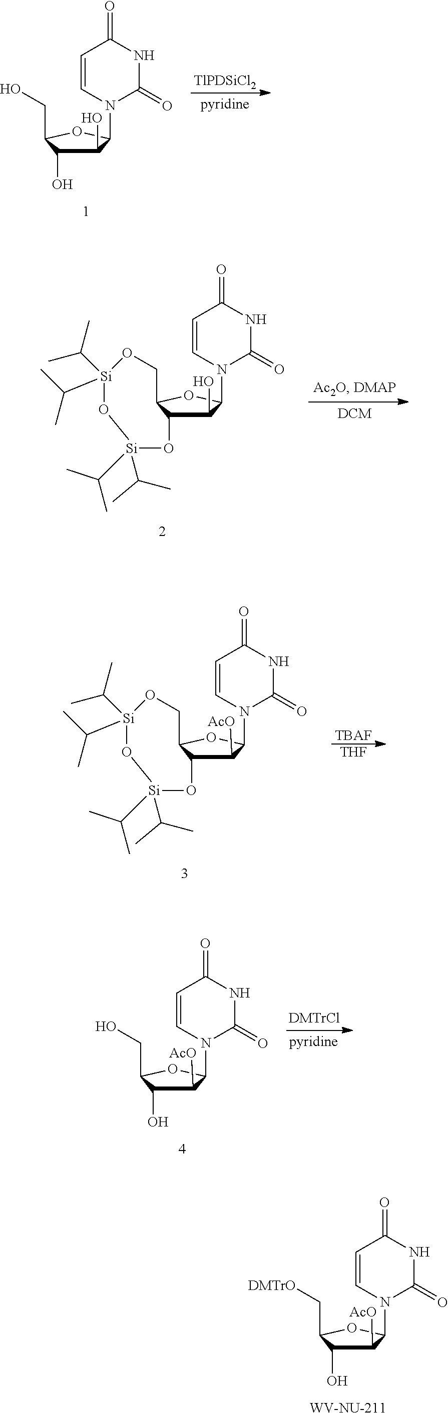

Step 1. To a solution of compound 1 (36 g, 147.42 mmol, 1 eq.) in pyridine (500 mL) at 0° C. drops added chloro-[chloro(diisopropyl)silyl]oxy-diisopropyl-silane (51.15 g, 162.16 mmol, 51.88 mL, 1.1 eq.) in N₂. And 2 hr latter, the mixture was stirred at 0-20° C. for 10 hr. LCMS showed compound 1 was consumed completely and desired mass was detected. The reaction liquid was vacuum concentrated to obtain crude product. The residue was purified by column chromatography (SiO₂. Petroleum ether:Ethyl acetate=0:1). Compound 2 (68 g, 139.71 mmol, 97.14% yield) was obtained as a white solid. ¹HNMR (400 MHz, CHLOROFORM-d) δ=10.08 (s, 1H), 7.85 (d, J=8.1 Hz, 1H), 6.10 (d, J=5.9 Hz, 1H), 5.70 (dd, J=1.1, 8.0 Hz, 1H), 4.62-4.51 (m, 2H), 4.20-4.08 (m, 2H), 4.05-3.97 (m, 1H), 3.76 (br d, J=8.9 Hz, 1H), 1.14-1.01 (m, 28H); LCMS (M+H⁺): 487.3; purity: 94.77%.

Step 2. To a solution of compound 2 (68 g, 139.71 mmol) in DCM (700 mL) and Py (37 mL) was added Ac₂O (17.12 g, 167.66 mmol) and DMAP (17.07 g, 139.71 mmol, 1 eq.). The mixture was stirred at 25° C. for 2 hr. TLC indicated compound 2 was consumed completely and one new spot formed. The reaction mixture was diluted with water 400 mL, and then separate and collect organic phases. The combined organic layers were dried over Na₂SO₄, filtered and concentrated under reduced pressure to give a residue. Compound 3 (73 g, crude) was obtained as a colorless oil. LCMS (M+H⁺):551.3.

Step 3. To a solution of compound 3 (73 g, 138.06 mmol) in THF (700 mL) was added TBAF (1 M, 207.10 mL) and AcOH (8.29 g, 138.06 mmol). The mixture was stirred at 20° C. for 2 hr. TLC indicated compound 3 was consumed completely and one new spot formed. The reaction mixture was filtered and concentrated under reduced pressure to give a residue. The residue was purified by column chromatography (SiO₂, Ethyl acetate:MeOH=20:1 to 5:1). After concentration under reduced pressure, 600 mL ethyl acetate was stirred for 10 min, and white solid was obtained by filtration. Compound 4 (12 g, 40.00% yield) was obtained as a white solid. ¹H NMR (400 MHz, DMSO-d6) δ=7.71 (d, J=8.1 Hz, 1H), 6.14 (d, J=5.3 Hz, 1H), 5.62 (d, J=8.0 Hz, 1H), 5.15 (t, J=4.8 Hz, 1H), 4.12 (t, J=5.0 Hz, 1H), 3.78 (q, J=4.8 Hz, 1H), 3.68-3.56 (m, 2H), 3.19-3.12 (m, 1H), 1.93 (s, 3H), 0.93 (t, J - 7.3 Hz, 1H); LCMS (M+H⁺): 309.1.

Step 4. To a solution of compound 3 (12 g, 41.92 mmol) in pyridine (120 mL) was added DMTCl (14.20 g, 41.92 mmol). The mixture was stirred at 25° C. for 16 hr. TLC indicated compound 4 was consumed completely and two new spots formed. The reaction mixture was filtered and concentrated under reduced pressure to give a residue. The residue was purified by column chromatography (SiO₂, Petroleum ether:Ethyl acetate=10:1 to 0:1, 5% TEA). Compound WV-NU-211 (19.8 g, 33.64 mmol, 82.50% yield) was obtained as a white solid. ¹HNMR (400 MHz, DMSO-d6) δ=11.37 (s, 1H), 7.47-7.20 (m, 11H), 6.91 (dd, J=1.8, 8.8 Hz, 4H), 6.17 (d, J=4.9 Hz, 1H), 5.90 (d, J=5.0 Hz, 1H), 5.45 (dd, J=1.6, 8.1 Hz, 1H), 5.10 (t, J=4.4 Hz, 1H), 4.13 (q, J=4.7 Hz, 1H), 3.74 (s, 6H), 3.31-3.19 (m. 2H), 1.83 (s, 3H); LCMS (M−H⁺):587.2; purity: 96.89%.

### Synthesis of (2R, 3S, 4R, 5R)-5-((bis(4-methoxyphenyl)(phenyl)methoxy)methyl)-2-(2, 6-dioxo-3, 6-dihydropyrimidin-1(2H)-yl)-4-hydroxytetrahydrofuran-3-yl acetate (WV-NU-212)

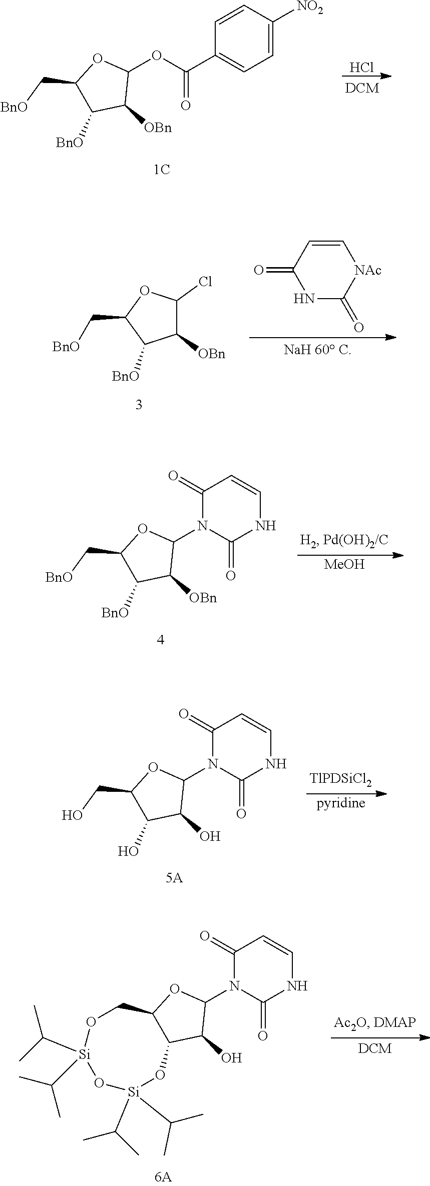

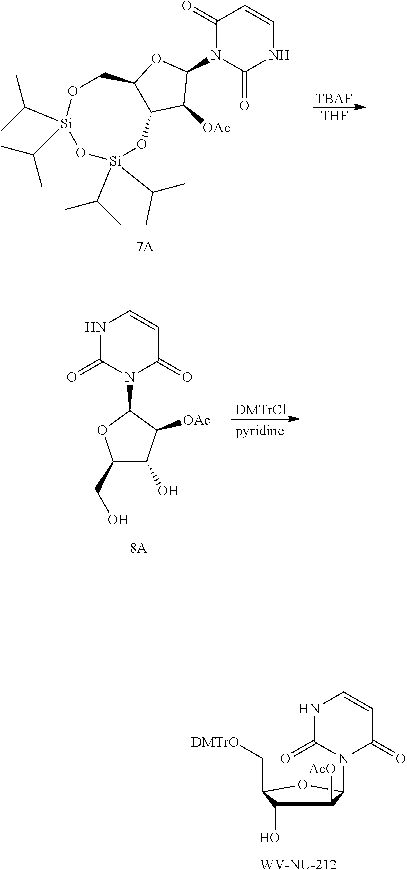

Step 1. For two batches: HCl (3.20 g, 87.78 mmol, 3.14 mL, 1 eq.) was bubbled into a solution of IC (50 g, 87.78 mmol. I eq.) in DCM (300 mL) at 0° C. for 0.3 hr. TLC (Petroleum ether:Ethyl acetate=3:1) indicated compound 1C was consumed completely and one new spot formed. The reaction was clean according to TLC. The reaction mixture was concentrated to give the crude product. Compound 3 (77 g, crude) was obtained as a colorless oil.

Step 2. For two batches: To a solution of 1-acetylpyrimidine-2, 4(1H, 3H)-dione (18.93 g, 122.80 mmol, 1.4 eq.) in MeCN (300 mL) Cool the reaction mixture to 0° C. by using ice bath, then, add NaH (5.26 g, 131.57 mmol, 60% purity, 1.5 eq.) portion wise to the reaction mixture and stir for 30 min at 0° C. After that, compound 3 (38.5 g, 87.71 mmol, 1 eq.) was added to the mixture. The mixture was stirred at 60° C. for 4 hrs. TLC (Petroleum ether:Ethyl acetate=1:1) indicated compound 3 was consumed completely. The reaction mixture was partitioned between H₂O 500 mL and EtOAc 3000 mL. The organic phase was separated, washed with brine 50 mL, dried over NA₂SO₄, filtered and concentrated under reduced pressure to give a residue. The residue was purified by column chromatography (SiO₂, Petroleum ether/Ethyl acetate=100/1 to 1/1) to get compound 4 (20, 38.48 mmol, 21.94% yield, 99% purity) as a white solid. ¹HNMR (CHLOROFORM-d, 400 MHz): δ=8.64 (br d, J=5.3 Hz, 1H), 7.20-7.41 (m, 17H), 6.79-6.97 (m, 2H), 5.62 (dd, J=7.7, 1.4 Hz, 1H), 4.40-4.71 (m, 8H), 4.08-4.17 (m, 1H), 3.96 (dd, J=10.2, 8.3 Hz, 1H), 3.72 ppm (dd, J=10.4, 2.8 Hz, 1H); LCMS: M+H⁺=515.

Step 3. To a solution of compound 4 (20 g, 38.87 mmol, 1 eq.) was dissolved in MeOH (600 mL). Addition of Pd (OH)₂ (3.20 g, 4.56 mmol, 20% purity, 1.17e-1 eq.) was followed by addition of HCl (1 M, 58.30 mL, 1.5 eq.). The reaction mixture was stirred vigorously under H₂ atmosphere (10 psi) at 15° C. for 4.5 hrs. TLC (Dichloromethane:Methanol=5:1) indicated Compound 4 was consumed completely and one new spot formed. The reaction was clean according to TLC. Concentrated in vacuum to give compound 5A (9.4 g, crude) as a colorless oil without further purification.

Step 4. To a solution of 5A (9 g, 36.85 mmol, 1 eq.) in pyridine (30 mL) was added chloro-[chloro(diisopropyl)silyl]oxy-diisopropyl-silane (13.95 g, 44.23 mmol, 14.15 mL, 1.2 eq.).The mixture was stirred at 15° C. for 12 hr. LC-MS showed compound 5A was consumed completely and one main peak with desired m/z was detected. The reaction mixture was partitioned between H₂O 20 mL and EtOAc 60 mL. The organic phase was separated, washed with brine 23 mL dried over Na₂SO₄, filtered and concentrated under reduced pressure to give a residue. The residue was purified by column chromatography (SiO₂, Petroleum ether/Ethyl acetate=100/1 to 1/1). Compound 6A (10 g, 20.55 mmol, 55.75% yield) was obtained as a white solid. ¹H NMR (CHLOROFORM-d, 400 MHz): δ=11.00 (br d, J=5.0 Hz, 1H), 7.38 (dd, J=7.5, 5.9 Hz, 1H), 6.39 (d, J=8.6 Hz, 1H), 5.47-5.55 (m, 2H), 4.81 (t, J=6.9 Hz, 1H), 4.32-4.41 (m, 1H), 4.16 (dd, J=10.9, 9.3 Hz, 1H), 3.76 (dd, J=11.0, 3.5 Hz, 1H), 3.56-3.63 (m, 1H), 0.95-1.12 ppm (m, 28H); LCMS (M+H⁺): 487.

Step 5. To a solution of Compound 6A (11.00 g, 22.60 mmol, 1 eq.) in DCM (200 mL) was added Ac2O (2.08 g, 20.34 mmol, 1.91 mL, 0.9 eq.) and DMAP (1.38 g, 11.30 mmol, 0.5 eq.). The mixture was stirred at 15° C. for 4 hr. TLC (Petroleum ether:Ethyl acetate=1:1) indicated compound 6A was consumed completely and one new spot formed. The reaction was clean according to TLC. The reaction mixture was partitioned between DCM 200 mL and H2O 100 mL. The organic phase was separated, washed with brine 5 mL, dried over NA₂SO₄, filtered and concentrated under reduced pressure to give a residue. The residue was purified by column chromatography (SiO₂, Petroleum ether/Ethyl acetate=100/1 to 0/1). Compound 7A (9 g, 17.02 mmol, 75.310% yield) was obtained as a white solid. ¹HNMR (CHLOROFORM-d, 400 MHz): δ=11.11 (br d, J=4.1 Hz, 1H), 7.41 (dd, J=7.3. 5.3 Hz, 1H), 6.62 (d, J=8.3 Hz, 1H), 5.52 (d, J=7.5 Hz, 1H), 5.32 (dd, J=8.2, 7.2 Hz, 1H), 5.15 (t, J=7.1 Hz, 1H), 4.12 (dd, J=11.0, 8.8 Hz, 1H), 3.86 (dd, J=11.2, 3.5 Hz, 1H), 3.68-3.80 (m, 1H), 1.85-1.91 (m, 3H), 0.89-1.12 ppm (m, 28H).

Step 6. To a solution of Compound 7A (9 g, 17.02 mmol, 1 eq.) in THF (100 mL) was added TBAF (1 M, 34.04 mL, 2 eq.). The mixture was stirred at 15° C. for 4 hr. TLC (Petroleum ether:Ethyl acetate =0:1) indicated compound 7A was consumed completely and one new spot formed. The reaction was clean according to TLC. The filtration was concentrated in vacuum to give a residue. The residue was purified by column chromatography (SiO₂, Petroleum ether/Ethyl acetate=100/1 to 1/1). Compound 8A (3 g, 10.48 mmol, 61.57% yield) was obtained as a white solid. ¹H NMR (DMSO-d₆, 400 MHz): δ=11.06 (br d, J=5.5 Hz, 1H), 7.28-7.62 (m, 1H), 6.62 (d, J=8.3 Hz, 1H), 5.53 (dd, J=7.5, 1.3 Hz, 1H), 5.05-5.22 (m, 1H), 4.38-4.58 (m, 1H), 3.55-3.71 (m, 3H), 1.91 ppm (s, 3H).

Step 7. To a solution of Compound 8A (2.9 g, 10.13 mmol, 1 eq.) in pyridine (50 mL) was added DMTCl (3.09 g, 9.12 mmol, 0.9 eq.). The mixture was stirred at 25° C. for 2 hr. TLC (Petroleum ether:Ethyl acetate=1:1) indicated compound 8A was consumed completely and (one new spot) formed. The reaction was (clean) according to TLC. The reaction mixture was partitioned between EtOAc 200 mL and H2O 100 mL. The organic phase was separated, washed with brine 50 mL, dried over NA₂SO₄, filtered and concentrated under reduced pressure to give a residue. The residue was purified by column chromatography (SiO₂, Petroleum ether/Ethyl acetate=100/1 to 0/1). (2R, 3S, 4R, 5R)-5-((Bis(4-methoxyphenyl)(phenyl)methoxy)methyl)-2-(2, 6-dioxo-3, 6-dihydropyrimidin-1(2H)-yl)-4-hydroxytetrahydrofuran-3-yl acetate (WV-NU-212) (2 g, 3.40 mmol, 33.54% yield) was obtained as a white solid. ¹H NMR (DMSO-d₆, 400 MHz): δ=11.04 (br s, 1H), 7.46 (br d, J=7.7 Hz, 1H), 7.39 (br d, J=7.3 Hz, 2H), 7.17-7.30 (m, 7H), 6.85 (t, J=9.3 Hz, 4H), 6.70 (d, J=8.3 Hz, 1H), 5.39-5.57 (m, 2H), 5.18 (t, J=7.7 Hz, 1H), 4.36-4.47 (m, 1H), 3.84-3.95 (m, 1H), 1.89 ppm (s, 3H); LCMS (M−H⁺): 587; LCMS purity: 96.899.

### Synthesis of N, N′-(9-((2R, 4S, 5R)-5-((bis(4-methoxyphenyl)(phenyl)methoxy)methyl)-4-hydroxytetrahydrofuran-2-yl)-9H-purine-2, 6-diyl)bis(2-methylpropanamide) (WV-NU-226A)

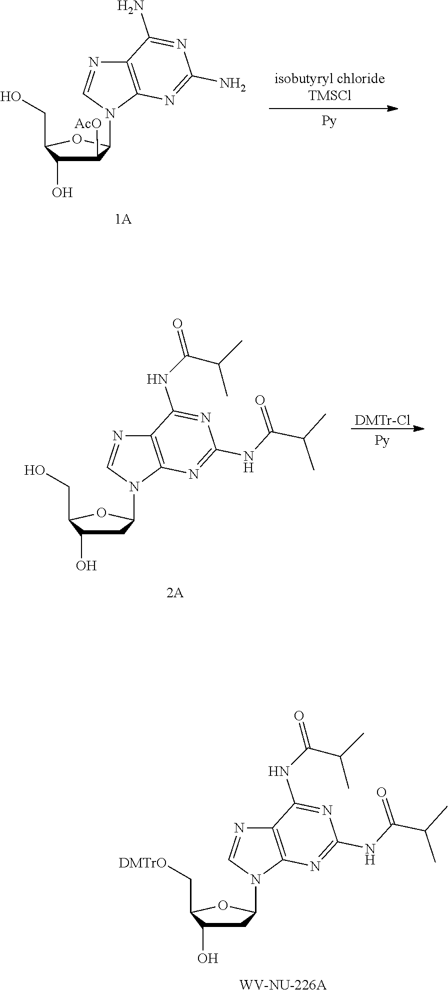

Step 1. To a solution of (2R, 3S, 5R)-5-(2, 6-diamino-9H-purin-9-yl)-2-(hydroxymethyl)tetrahydrofuran-3-ol (1A) (10.5 g, 39.44 mmol) in Py (500 mL) and then TMSCl (39.90 g, 367.26 mmol) was added and the solution was stirred at 25° C. for 30 min. Then, 2-methylpropanoyl chloride (34.02 g, 319.29 mmol) was added drop wise, and the reaction mixture was stirred at 25° C. for 3 hr. LCMS showed compound 1A was consumed completely and desired mass was detected. The reaction mixture was cooled in an ice-bath, H₂O (100 mL) was added, and subsequently, 5 min later, 28% aq. ammonia (100 mL) was introduced. Stirring was continued for 30 min at 25° C. The solvent from the reaction mixture was evaporated, and the remaining oily residue was evaporated with toluene (3×100 mL) and got the crude oil product. The residue was purified by column chromatography (SiO₂, Petroleum ether:Ethyl acetate to Ethyl acetate: MeOH=1:1 to 5:1). N, N′-(9-((2R, 4S, 5R)-4-hydroxy-5-(hydroxymethyl)tetrahydrofuran-2-yl)-9H-purine-2, 6-diyl)bis(2-methylpropanamide) (2A) (8.2 g, 51.25% yield) was obtained as a white solid. ¹HNMR (400 MHz, DMSO-d6) δ=10.50 (s, 1H), 10.34 (s, 1H), 8.51 (s, 1H), 6.35 (t, J=6.8 Hz, 1H), 5.32 (d, J=4.0 Hz, 1H), 4.90 (t, J=5.6 Hz, 1H), 4.43 (br d, J=2.3 Hz, 1H), 4.09 (q, J=5.3 Hz, 1H), 3.89-3.83 (m, 1H), 3.63-3.49 (m, 2H), 3.03 (td, J=6.8, 13.6 Hz, 1H), 2.95-2.85 (m, 1H), 2.74 (td, J=6.6, 13.3 Hz, 1H), 2.30 (ddd, J=3.3, 6.1, 13.2 Hz, 1H), 1.10 (dd, J=6.8, 12.3 Hz, 12H); LCMS (M+H*): 407.2.

Step 2. To a solution of N, N′-(9-((2R, 4S, 5R)-4-hydroxy-5-(hydroxymethyl)tetrahydrofuran-2-yl)-9H-purine-2, 6-diyl)bis(2-methylpropanamide) (2A) (8 g, 19.68 mmol) in Py (100 mL) was added DMT-Cl (7.34 g, 21.65 mmol) in N₂. The mixture was stirred at 25° C. for 4 hr. LCMS showed compound 2A was consumed completely and desired mass was detected. The reaction mixture was filtered and concentrated under reduced pressure to give a residue. The crude product was purified by reversed-phase HPLC (column: Phenomenex C18 250*100 mm 10u; mobile phase: [water (NH₄HCO₃)-ACN]; B %: 45%-75%, 20 min). N, N′-(9-((2R, 4S, 5R)-5-((Bis(4-methoxyphenyl)(phenyl)methoxy)methyl)-4-hydroxytetrahydrofuran-2-yl)-9H-purine-2, 6-diyl)bis(2-methylpropanamide) (WV-NU-226A) (3.4 g, 34.00% yield) was obtained as a white solid. ¹HNMR (400 MHz. DMSO-d6) δ=10.50 (s, 1H), 10.27 (s, 1H), 8.39 (s, 1H). 7.33-7.27 (m, 2H), 7.23-7.12 (m, 7H), 6.82-6.72 (m, 4H), 6.39 (t, J=6.4 Hz, 1H), 5.33 (d, J=4.6 Hz, 1H), 4.53 (quin, J=5.0 Hz, 1H), 4.10 (q, J=5.3 Hz, 1H), 3.99-3.93 (m, 1H), 3.70 (d, J=3.0 Hz, 6H), 3.32-3.26 (m, 1H), 3.05 (td, J=6.8, 13.6 Hz, 1H), 2.87 (qd, J=6.3, 12.9 Hz, 2H), 2.41-2.32 (m, 1H), 1.12 (d, J=6.8 Hz, 6H), 1.05 (t, J=7.4 Hz, 6H); LCMS (M−H⁺): 707.4, purity: 100.0%.

### Synthesis of N-(5-((2R, 4S, 5R)-5-((bis(4-methoxyphenyl)(phenyl)methoxy)methyl)-4-hydroxytetrahydrofuran-2-yl)-3-nitro-6-(4-nitrophenethoxy)pyridin-2-yl)acetamide (WV-NU-225)

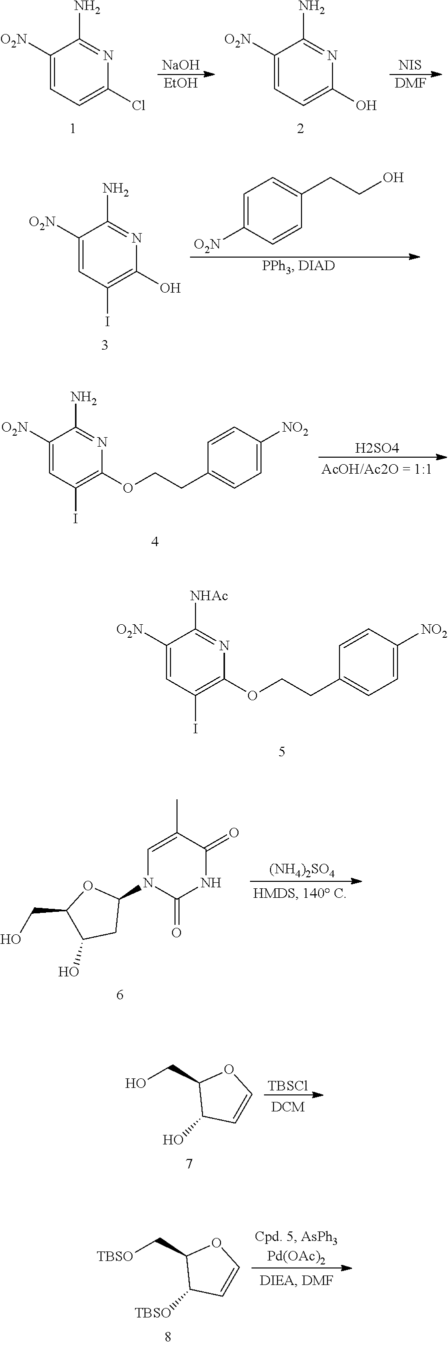

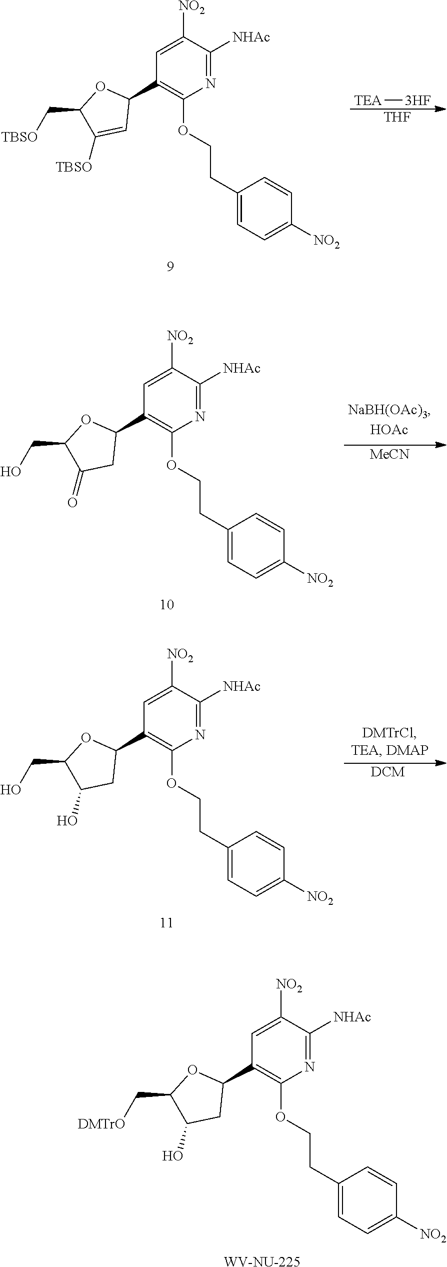

Step 1. To a suspension of compound 1 (50 g, 288.09 mmol.) in refluxing EtOH (800 mL) and H₂O (300 mL) was added NaOH (10 M, 182.65 mL). The mixture was stirred at 110° C. for 5 min. The reaction mixture turned to clear and the color from yellow turned to orange. The resulting orange solution was cooled on ice. Acidification with aqueous 6 M HCl led to a large amount of yellow precipitate. Filtration and the solid was desired product. Compound 2 (44 g, crude) was obtained as a yellow solid.

Step 2. Compound 2 (44 g, 283.67 mmol.) was rendered anhydrous by co-evaporation with DMF under high vacuum and then resuspended in DMF (450 mL), NIS (95.73 g, 425.50 mmol) was added and the resulting red, clear solution was stirred at 15° C. for 20 hr in the dark. LCMS showed the desired mass was detected. The reaction solution was slowly added to vigorously stirred water (4 L) and the resulting suspension was filtered and the solid is desired. Compound 3 (59 g, crude) was obtained as a yellow solid. LCMS (M−H⁺): 279.9.

Step 3. To a solution of compound 3 (59 g, 209.96 mmol) in THF (1500 mL) was added 2-(4-nitrophenyl) ethanol (44.25 g, 264.71 mmol), PPh₃ (82.60 g, 314.94 mmol, 1.5 eq.) and DEAD (54.85 g, 314.94 mmol.). The mixture was stirred at 20° C. for 12 hr. LCMS showed the desired mass was detected. The reaction mixture was concentrated under reduced pressure to remove solvent. The residue was purified by flash chromatography (silica, Petroleum ether: DCM=1/0 to 0/1) to give a yellow solid, then it was treated with Petroleum ether/EtOAc (600 mL/200 mL) and the insoluble solid was filtered and dried. Compound 4 (30 g, 33.22% yield) was obtained as a yellow solid. LCMS (M−H⁺): 429.1.

Step 4. To a solution of compound 4 (15 g, 34.87 mmol) in AcOH (120 mL) and Ac₂O (120 mL) was added H₂SO₄ (1.71 g, 17.44 mmol). The mixture was stirred at 65° C. for 1 hr. LCMS showed compound 4 was consumed completely and one main peak with desired mass was detected. The reaction mixture was cooled to room temperature and was neutralized by sat.NaHCO₃, and then the mixture was extracted with ethyl acetate (300 mL*3). The combined organic layers were washed with brine 200 mL, dried over Na₂SO₄, filtered and concentrated under reduced pressure to give a residue. The residue was purified by column chromatography (SiO₂, Petroleum ether/Ethyl acetate=1/0 to 0/1). Compound 5 (15 g, 91.10% yield) was obtained as a yellow solid. LCMS (M+H⁺): 473.1

Step 5. Compound 6 (100 g, 412.83 mmol) was introduced to a dried flask under Ar. HMDS (1000 mL) was added, and the solution was stirred until dissolved. After the addition of the (NH₄)₂SO₄ (87.28 g, 660.53 mmol), the solution was refluxed 140° C. for 4 h. TLC showed the starting material was consumed several spots were shown on TLC, filtered and concentrated under reduced pressure to give a residue. The residue was purified by column chromatography (SiO₂, Petroleum ether/Ethyl acetate=100/1 to 0/1) to get compound 7 (21 g, 43.81% yield) was obtained as a yellow oil. ¹HNMR (CHLOROFORM-d, 400 MHz): δ=6.48-6.53 (m, 1H), 5.03 (t, J=2.6 Hz, 1H), 4.82 (dt, J=2.6, 1.3 Hz, 1H), 4.32 (td, J=6.5, 2.7 Hz, 1H), 3.66 (dd, J=10.7, 6.2 Hz, 1H), 3.45 ppm (dd, J=10.7, 6.8 Hz, 1H).

Step 6. To a solution of compound 7 (21 g, 180.86 mmol) in DCM (1000 mL) was added TBSCl (81.78 g, 542.57 mmol) and IMIDAZOLE (61.56 g, 904.28 mmol.). The mixture was stirred at 15° C. for 13 hr. TLC indicated compound 7 was consumed completely and two new spots formed. The reaction was clean according to TLC. Filtered and concentrated under reduced pressure to give a residue. The residue was purified by column chromatography (SiO₂, Petroleum ether/Ethyl acetate=100/1 to 5/1) to get compound 8 (21 g, 33.69% yield) was obtained as a yellow oil. ¹HNMR (CHLOROFORM-d, 400 MHz): δ=6.59-6.67 (m, 1H), 5.05 (t, J=2.6 Hz, 1H), 4.79-4.85 (m, 1H), 4.09-4.21 (m, 1H), 3.59-3.68 (m, 1H), 3.45 (dd, J=10.9, 6.4 Hz, 1H), 0.83-0.88 (m, 18H), 0.83-0.89 (m, 1H), 0.02-0.09 ppm (m, 12H).

Step 7. BSA (33.39 g, 164.13 mmol) was added dropwise to a suspension of compound 8 (19.74 g, 57.28 mmol.) in DMF (500 mL) under N₂ atmosphere. After stirring for 1 h the reaction become a clear solution. Then DIEA (21.21 g, 164.13 mmol) and compound 5 (25 g, 52.94 mmol, 1 eq.) were added. In a separate flask, triphenylarsane (3.24 g, 10.59 mmol) and diacetoxypalladium (1.19 g, 5.29 mmol) were dissolved in DMF (50 mL) and the mixture was stirred at 20° C. for 30 min. Then this mixture was added slowly to the first flask and the mixture was stirred for 12 hr at 80° C. LCMS showed the desired mass was detected. The reaction was quenched with the addition of H₂O (600 mL) and the solvent was evaporated under reduced pressure. The residue was re-dissolved in EtOAc (500 mL), and washed with H2O (2*500 mL) and brine (500 mL). The organic layer was dried over Na₂SO₄, filtered and concentrated under reduced pressure. The residue was purified by column chromatography (SiO2, Petroleum ether/Ethyl acetate=1/0 to 0/1). Compound 9 (11 g, 30.16% yield) was obtained as a brown gum. ¹HNMR (400 MHz, CHLOROFORM-d) δ=10.61 (s, 1H), 8.67 (s, 1H), 8.20 (d, J=8.6 Hz, 2H), 7.49 (d, J=8.6 Hz, 2H), 5.82-5.71 (m, 1H), 4.75 (t, J=6.4 Hz, 2H), 4.67 (s, 1H), 4.62-4.56 (m, 1H), 3.91 (dd, J=1.8, 11.3 Hz, 1H), 3.73 (dd, J=3.9, 11.3 Hz, 1H), 3.33-3.22 (m, 2H), 2.46 (s, 3H), 0.94 (s, 9H), 0.82 (s, 9H), 0.17 (d, J=15.8 Hz, 6H), 0.02 (d, J=2.1 Hz, 6H); LCMS (M−H⁺): 687.4.

Step 8. To a solution of compound 9 (10.8 g, 15.68 mmol) in THF (100 mL) was added N, N-diethylethanamine;trihydrofluoride (25.27 g, 156.77 mmol). The mixture was stirred at 60° C. for 2 hr under N₂. LCMS showed the desired mass was detected. The reaction mixture was concentrated under reduced pressure to remove solvent. The crude product was washed with water 100 mL*2. Compound 10 (7.22 g, crude) was obtained as a brown solid. LCMS (M−H⁺): 459.3.

Step 9. To a solution of compound 10 (7.22 g, 15.68 mmol.) in ACN (100 mL) was added NaBH(OAc)₃ (13.29 g, 62.73 mmol). The mixture was stirred at 20° C. for 5 hr. LCMS showed the desired mass was detected. The reaction mixture was concentrated under reduced pressure to remove solvent. The residue was diluted with water 100 mL and extracted with ethyl acetate 300 mL (100 mL*3). The combined organic layers were washed with brine 100 mL, dried over Na₂SO₄, filtered and concentrated under reduced pressure to give a residue. The residue was purified by column chromatography (SiO₂, Petroleum ether/Ethyl acetate=1/0 to 0/1). Compound 11 (6 g, 82.74% yield) was obtained as a yellow solid. ¹H NMR (400 MHz, DMSO-d₆) δ=10.75 (s, 1H), 8.29 (s, 1H), 8.18 (d, J=8.6 Hz, 2H), 7.61 (d, J=8.6 Hz, 2H), 5.09 (d, J=4.0 Hz, 1H), 5.00 (dd, J=5.7, 9.7 Hz, 1H), 4.81 (t, J=5.4 Hz, 1H), 4.70-4.54 (m, 2H), 4.12 (br d, J=1.9 Hz, 1H), 3.78 (dt, J=2.4, 4.8 Hz, 1H), 3.48-3.43 (m, 2H), 3.23 (br t, J=6.4 Hz, 2H), 2.10 (s, 3H), 2.04 (ddd, J=1.9, 5.8, 12.6 Hz, 1H), 1.58 (ddd, J=5.8, 9.8, 12.7 Hz, 1H); LCMS (M−H⁺): 461.3.

Step 10. To a solution of compound 11 (6 g, 12.98 mmol) in DCM (100 mL) was added TEA (13.13 g, 129.76 mmol), DMAP (792.60 mg, 6.49 mmol.) and DMTCl (5.28 g, 15.57 mmol.). The mixture was stirred at 30° C. for 20 hr. LCMS showed the desired mass was detected. The reaction mixture was quenched by addition methanol 20 mL at 20° C., and then was concentrated under reduced pressure to give a residue. The residue was purified by column chromatography (SiO₂, Petroleum ether/Ethyl acetate=I/O to 0/1). N-(5-((2R, 4S, 5R)-5-((Bis(4-methoxyphenyl)(phenyl)methoxy)methyl)-4-hydroxytetrahydrofuran-2-yl)-3-nitro-6-(4-nitrophenethoxy)pyridin-2-yl)acetamide (WV-NU-225) (7.6 g, 75.35% yield) was obtained as a yellow solid. ¹HNMR (400 MHz, CHLOROFORM-d) δ=10.59 (s, 1H), 8.61 (s, 1H), 8.19 (d, J=8.8 Hz, 2H), 7.48 (d, J=8.8 Hz, 2H), 7.42 (d, J=7.4 Hz, 2H), 7.35-7.28 (m, 5H), 7.22 (d, J=7.3 Hz, 1H), 6.84 (d, J=8.5 Hz, 4H), 5.13 (dd, J=5.6, 9.9 Hz, 1H), 4.72 (t, J=6.6 Hz, 2H), 4.42-4.35 (m, 1H), 4.09-4.02 (m, 1H), 3.80 (s, 6H), 3.40-3.34 (m, 1H), 3.31-3.23 (m, 3H), 2.46 (s, 3H), 2.19 (ddd, J=1.7, 5.7, 13.0 Hz, 1H), 1.75 (ddd, J=6.1, 10.0, 13.0 Hz, 1H); LCMS (M−H⁺): 763.3; purity: 98.38%.

### Synthesis of 3-((2R, 4S, 5R)-5-((bis(4-methoxyphenyl)(phenyl)methoxy)methyl)-4-hydroxytetrahydrofuran-2-yl)-5-bromopyrimidine-2, 4(1H, 3H)-dione (WV-NU-238)

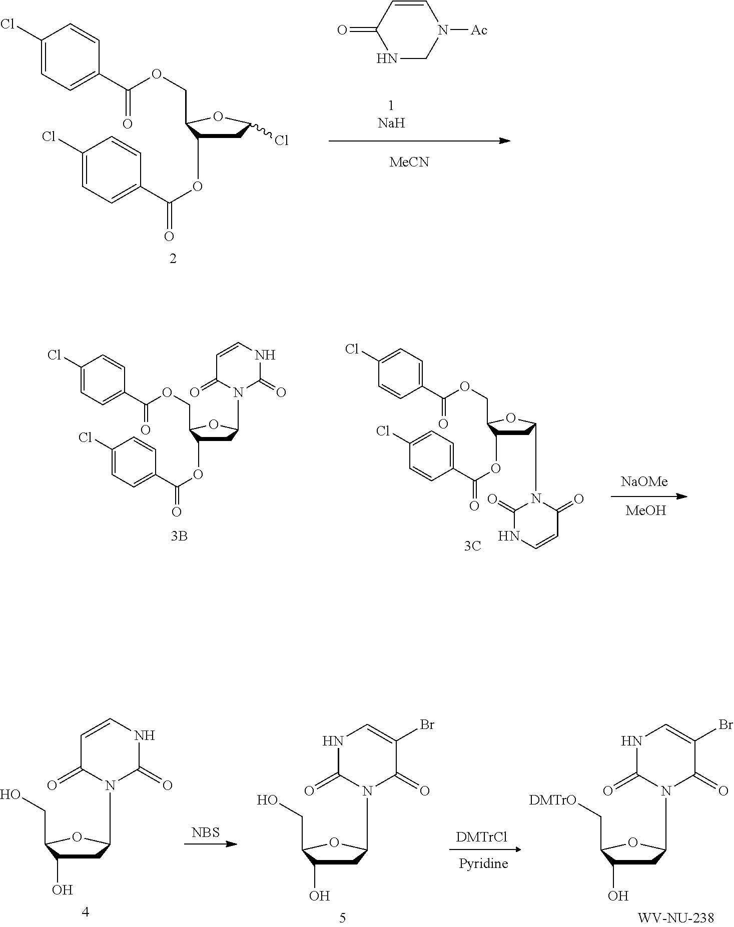

Step 1. For four batches: a clean and dry three-neck 3 Lit round bottom flask charge with 1-acetylpyrimidine-2, 4(1H, 3H)-dione (17 g, 110.30 mmol, 1 eq.) and dissolved into dry ACN (1700 mL) under argon atmosphere. Cool the reaction mixture to 0° C. by using ice bath. Then NaH (6.62 g, 165.45 mmol, 60% purity, 1.5 eq.) was added portion wise to the reaction mixture and stir for 30 min at 0° C. After that, compound 2 (66 g, 153.60 mmol, 1.39 eq.) was added portion wise and stir the reaction mixture for 30 min at 0° C. and 65° C. for 3 h. TLC (Petroleum ether:Ethyl acetate=1:1, Rf=0.25) showed that compound 2 was consumed and new spots was formed. Cool the reaction mixture to r.t. and filtered, the filtrate of four batches were combined and concentrated under reduced pressure to give a crude product. The residue was purified by column chromatography (SiO₂, Petroleum ether/Ethyl acetate=1/0 to 1/2), then was triturated with Ethyl acetate (500 mL) and MeCN (50 mL) at 60° C. for 15 min, filtered and the cake re-triturated for three times, the cake was dried under vacuum to get the product and all the mother liquor was concentrated to get the crude. ((2R, 3S, 5R)-3-((4—Chlorobenzoyl)oxy)-5-(2, 6-dioxo-3, 6-dihydropyrimidin-1(2H)-yl)tetrahydrofuran-2-yl)methyl 4-chlorobenzoate (3B) (25 g, 49.48 mmol, 11.21% yield) was obtained as a white solid and there was 40 g crude in hand.

Step 2. To a solution of ((2R, 3S, 5R)-3-((4-chlorobenzoyl)oxy)-5-(2, 6-dioxo-3, 6-dihydropyrimidin-1(2H)-yl)tetrahydrofuran-2-yl)methyl 4-chlorobenzoate (3B) (20 g, 39.58 mmol, 1 eq.) in MeOH (200 mL) was added NaOMe (5.35 g, 98.95 mmol, 2.5 eq.). The mixture was stirred at 15° C. for 2 hr. TLC (Petroleum ether:Ethyl acetate=0:1, Rf=0.05) indicated compound 3B was consumed completely and two new spots formed. The reaction mixture was quenched by addition NH₄Cl 5.35 g, and then concentrated under reduced pressure to give a residue. The residue was purified by column chromatography (SiO₂, Petroleum ether/Ethyl acetate=1/1 to 0/1, then Ethyl acetate/methanol=1/0 to 5/1). 3-((2R, 4S, 5R)-4—Hydroxy-5-(hydroxymethyl)tetrahydrofuran-2-yl)pyrimidine-2, 4(1H, 3H)-dione (4) (8 g, 35.06 mmol, 88.57% yield) was obtained as a white solid.

Step 3. To a solution of 3-((2R, 4S, 5R)-4-hydroxy-5-(hydroxymethyl)tetrahydrofuran-2-yl)pyrimidine-2, 4(1H, 3H)-dione (4) (6.4 g, 28.05 mmol, 1 eq.) in THF (100 mL) was added NBS (5.99 g, 33.65 mmol. 1.2 eq.). The mixture was stirred at 20° C. for 6 hr. The reaction mixture was concentrated under reduced pressure to remove solvent. The residue was diluted with water 200 mL and extracted with DCM 40 mL (200 mL*2). The combined organic layers were washed with brine 300 mL, dried over Na₂SO₄, filtered and concentrated under reduced pressure to give a residue. The residue was purified by column chromatography (SiO₂, Ethyl acetate:Methanol=1/1 to 5/1). 5—Bromo-3-((2R, 4S, 5R)-4-hydroxy-5-(hydroxymethyl)tetrahydrofuran-2-yl)pyrimidine-2, 4(1H, 3H)-dione (5) (4.7 g, 15.30 mmol, 54.57% yield) was obtained as a yellow solid. LCMS (M−H⁺): 305.0

Step 4. To a solution of 5-bromo-3-((2R, 4S, 5R)-4-hydroxy-5-(hydroxymethyl)tetrahydrofuran-2-yl)pyrimidine-2. 4(1H, 3H)-dione (5) (4.7 g, 15.30 mmol, 1 eq.) in Py (40 mL) was added DMTCl (5.96 g, 17.60 mmol, 1.15 eq.). The mixture was stirred at 25° C. for 2 hr. The reaction mixture was quenched by addition NaHCO₃ 20 mL, and then diluted with water 50 mL and extracted with ethyl acetate 150 mL (50 mL *3). The combined organic layers were washed with brine 100 mL (50 mL*2), dried over Na₂SO₄, filtered and concentrated under reduced pressure to give a residue. The residue was purified by column chromatography (SiO₂, Petroleum ether/Ethyl acetate=1/0 to 0/1, 5% TEA). 3-((2R, 4S, 5R)-5-((Bis(4-methoxyphenyl)(phenyl)methoxy)methyl)-4-hydroxytetrahydrofuran-2-yl)-5-bromopyrimidine-2, 4(1H, 3H)-dione (WV-NU-238) (5.7 g, 9.06 mmol, 59.18% yield, 96.84% purity) was obtained as a yellow solid. ¹H NMR (400 MHz, DMSO-d₆) δ=11.51 (br s, 1H), 7.99 (s, 1H), 7.41-7.35 (m, 2H), 7.29-7.22 (m, 6H), 7.21-7.16 (m, 1H), 6.91-6.77 (m, 4H), 6.58 (dd, J=4.1, 9.0 Hz, 1H), 5.09 (d, J=5.5 Hz, 1H), 4.31-4.21 (m, 1H), 3.86-3.78 (m, 1H), 3.73 (d, J=1.8 Hz, 6H), 3.22 (dd, J=8.0, 9.6 Hz, 1H), 3.07 (dd, J=3.1, 9.7 Hz, 1H), 2.59 (ddd, J=4.1, 8.4, 13.0 Hz, 1H), 2.08-2.00 (m, 1H); LCMS (M−H⁺): 607.2; purity: 96.84%.

### Synthesis of 1-(9-((2R, 4S, 5R)-5-((bis(4-methoxyphenyl)(phenyl)methoxy)methyl)-4-hydroxytetrahydrofuran-2-yl)-8-oxo-8, 9-dihydro-7H-purin-6-yl)-3-phenylurea (WV-NU-234)

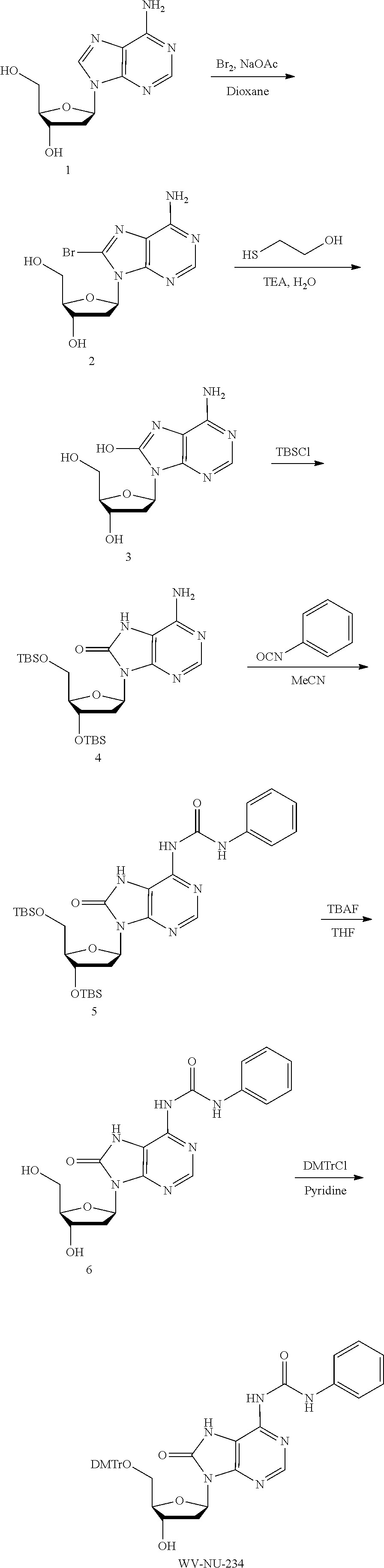

Step 1. For two batches: To a solution of (2R, 3S, 5R)-5-(6-amino-9H-purin-9-yl)-2-(hydroxymethyl)tetrahydrofuran-3-ol (50 g, 199.01 mmol, 1 eq.) in dioxane (400 mL) and AcONa (0.5 M, 1.87 L, 4.71 eq.) buffer (pH 4.3), a solution of Br₂ (38.16 g, 238.81 mmol, 12.31 mL, 1.2 eq.) was added dropwise while stirring. The mixture was stirred at 15° C. for 12 h. The two batches were combined for work up. To the mixture conc. Na₂S₂O₅ was added until the red color vanished. The mixture was neutralized to pH 7.0 with 0.5m NaOH. The residue was evaporated, when a white solid precipitated. The solid was filtered off, washed with cold 1, 4-dioxane(50 mL), and dried under high vacuum to get (2R, 3S, 5R)-5-(6-amino-8-bromo-9H-purin-9-yl)-2-(hydroxymethyl)tetrahydrofuran-3-ol (110 g, 333.19 mmol, 83.71% yield) as a yellow solid. ¹HNMR (400 MHz, DMSO-d6) δ=8.22-7.98 (m, 1H), 7.53 (br s, 2H), 6.29 (dd, J=6.5, 7.9 Hz, 1H), 5.35 (br d, J=12.3 Hz, 2H), 4.58-4.38 (m, 1H), 3.95-3.82 (m, 1H), 3.65 (dd, J=4.5, 11.9 Hz, 1H), 3.48 (br dd, J=4.5, 11.7 Hz, 1H), 3.36 (br s, 1H), 3.24 (ddd, J=6.1, 7.8, 13.4 Hz, 1H), 2.19 (ddd, J=2.6, 6.4, 13.1 Hz, 1H); LCMS:(M+H⁺):330.14.

Step 2. For two batches: A solution of (2R, 3S, 5R)-5-(6-amino-8-bromo-9H-purin-9-yl)-2-(hydroxymethyl)tetrahydrofuran-3-ol (55 g, 166.60 mmol, 1 eq.) 2-MERCAPTOETHANOL (39.22 g, 501.90 mmol, 35.01 mL, 3.01 eq.) and TEA (168.58 g, 1.67 mol, 231.88 mL, 10 eq. ) in water (1500 mL) was stirred under 110° C. for 4 hr. The solvent was removed under reduced pressure to give a residue which was purified by MPLC (Dichloromethane:Methanol=5:1, 10:1) to get 6-amino-9-((2R, 4S, 5R)-4-hydroxy-5-(hydroxymethyl)tetrahydrofuran-2-yl)-9H-purin-8-ol (65 g, 243.23 mmol, 73.00% yield) as a white solid. LCMS:(M+H⁺):267.24

Step 3. For three batches: To a solution of compound 3 (15 g, 56.13 mmol, 1 eq.) in DCM (300 mL) was added DMAP (3.43 g, 28.06 mmol, 0.5 eq.), TBSCl (33.84 g, 224.52 mmol, 27.51 mL, 4 eq.) and imidazole (19.11 g, 280.65 mmol, 5 eq.). The mixture was stirred at 15° C. for 12 hr. TLC (Petroleum ether: Ethyl acetate=0:1) indicated compound 3 was consumed completely and one new spot formed. Water (15 ml) and extracted with DCM 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0/1). Compound 4 (30 g, 60.51 mmol, 53.91% yield) was obtained as a white solid. LCMS (M+H⁺): 496.3.

Step 4. To a solution of compound 4 (10.1 g, 20.37 mmol, 1 eq.) in MeCN (200 mL), and isocyanatobenzene (7.28 g, 61.12 mmol, 6.62 mL*3 eq.) was added to the mixture, the solution was stirred at 20° C. for 12 hr. The reaction mixture was concentrated to get the crude. The mixture was purified by MPLC (SiO₂, Petroleum ether:Ethylacetate=30:1, 20:1, 10:1). Compound 5 (10 g, crude) as a yellow oil. ¹H NMR (400 MHz, DMSO-d6) δ=10.51 (s, 1H), 10.20 (br s, 1H), 9.41 (br s, 1H), 8.53 (s, 1H), 8.31 (s, 1H), 7.45 (d, J=7.9 Hz, 2H), 7.33 (d, J=7.8 Hz, 2H), 7.26-7.12 (m, 4H), 6.96 (t, J=7.4 Hz, 1H), 6.85 (t, J=7.3 Hz, 1H), 6.06 (t, J=6.9 Hz, 1H), 4.64-4.59 (m, 1H), 3.72-3.62 (m, 2H), 3.72-3.62 (m, 1H), 3.49 (dd, J=4.3, 10.1 Hz, 1H), 3.13-3.05 (m, 1H), 2.12-1.88 (m, 2H), 1.12 (s, 5H), 0.79 (s, 10H), 0.71 (s, 10H); LCMS (M+H⁺): 615.4.

Step 5. To a solution of compound 5 (10 g, 16.26 mmol, 1 eq.) in THF (100 mL) was added TBAF (1 M, 32.53 mL, 2 eq.) and the mixture was stirred at 20° C. for 0.5 hr. The mixture was concentrated to get the crude. The mixture was purified by MPLC (SiO₂, Petroleum ether:Ethyl acetate=5:1, 1:1, 0:1) to get compound 6 (6 g, crude) as a white solid. ¹HNMR (400 MHz, DMSO-d6) δ=11.85 (br s, 1H), 8.19 (s, 1H), 7.85 (s, 1H), 7.51 (d, J=7.6 Hz, 2H), 7.20 (t, J=7.9 Hz, 2H), 6.91 (t, J=7.4 Hz, 1H), 6.14 (dd, J=6.8, 8.0 Hz, 1H), 4.38-4.30 (m, 2H), 3.72 (hr d, J=2.4 Hz, 1H), 3.55 (hr d, J=4.5 Hz, 2H), 3.38 (br dd, J=4.5, 11.8 Hz, 1H), 2.98-2.88 (m, 2H), 1.92 (dt, J=2.5, 6.5 Hz, 1H); LCMS (M+H⁺): 387.2.

Step 6. For two batches. To a solution of compound 6 (2.5 g, 6.47 mmol, 1 eq.) in PYRIDINE (30 mL) was added DMTCl (2.19 g, 6.47 mmol, 1 eq.). The mixture was stirred at 15° C. for 0.5 hr. TLC (Dichloromethane:Methanol=20:1) indicated of compound 6 was remained, and many new spots formed. 2 reactions were combined for work up. The reaction mixture was quenched by addition water 50 mL at 15° C., and extracted with ethyl acetate 150 mL (50 mL *3). The combined organic layers were dried over Na₂SO₄, filtered and concentrated under reduced pressure to give a residue. The residue was purified by column chromatography (SiO2, DCM: MeOH=1:0 to 0:1). WV-NU-234(4.4 g, 6.39 mmol, 49.38% yield) was obtained as a yellow solid and WV-NU-234 (2 g, 5.18 mmol, 80.00% yield). The crude product was purified by reversed-phase HPLC (column:Welch Xtimate 250*70 mm #10 um 100A 5 g; mobile phase: [water-ACN]; B %: 45%-70% @20 mL/min), 1-(9-((2R, 4S, 5R)-5-((bis(4-methoxyphenyl)(phenyl)methoxy)methyl)-4-hydroxytetrahydrofuran-2-yl)-8-oxo-8, 9-dihydro-7H-purin-6-yl)-3-phenylurea (WV-NU-234) (4 g, 5.81 mmol, 80.00% yield) was obtained as a yellow solid. ¹H NMR (400 MHz, DMSO-d6) δ=10.65 (br s, 1H), 10.32 (br s, 1H), 9.52 (br s, 1H), 8.26 (br s, 1H), 7.56 (br d, J=6.9 Hz, 2H), 7.34 (br s, 4H), 7.21 (br d, J=6.3 Hz, 7H), 7.07 (br s, 1H), 6.83-6.72 (m, 4H), 6.22 (br s, 1H), 5.28 (br s, 1H), 4.50 (br s, 1H), 4.07-3.91 (m, 2H), 3.70 (br d, J=8.1 Hz, 6H), 3.24-2.95 (m, 2H), 2.14 (br s, 1H); LCMS (M−H⁺):688.73, purity: 98.33%.

### Synthesis of 3-((2R, 4S, 5R)-5-((bis(4-methoxyphenyl)(phenyl)methoxy)methyl)-4-hydroxytetrahydrofuran-2-yl)-4-thioxo-3, 4-dihydropyrimidin-2(1H)-one (WV-NU-237)

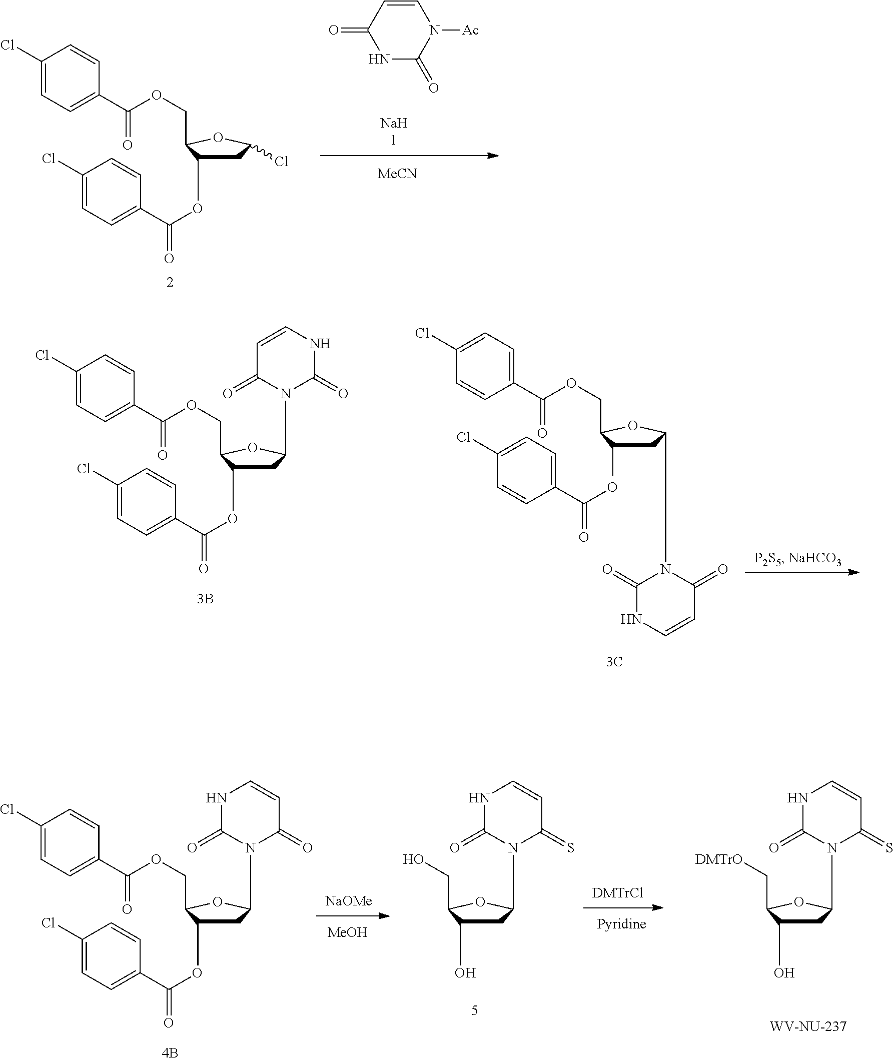

Step 1. For four batches: A clean and dry three-neck 3 Lit round bottom flask charge with compound 1 (17 g, 110.30 mmol, 1 eq.) and dissolved into dry ACN (1700 mL) under argon atmosphere. Cool the reaction mixture to 0° C. by using ice bath. Then NaH (6.62 g, 165.45 mmol, 60% o purity, 1.5 eq.) was added portion wise to the reaction mixture and stir for 30 min at 0° C. After that, compound 2 (66 g, 153.60 mmol, 1.39 eq.) was added portion wise and stir the reaction mixture for 30 min at 0° C. and 65° C. for 3 h. TLC (Petroleum ether:Ethyl acetate=1:1, Rf=0.25) show that compound 2 was consumed and new spots was formed. Cool the reaction mixture to r.t. and filtered, the filtrate of four batches were combined and concentrated under reduced pressure to give a crude product. The residue was purified by column chromatography (SiO₂, Petroleum ether/Ethyl acetate=1/0 to 1/2), then was triturated with Ethyl acetate (500 mL) and MeCN (50 mL) at 60° C. for 15 min, filtered and the cake re-triturated for three times, the cake was dried under vacuum to get the product and all the mother liquor was concentrated to get the crude. Compound 3B (49 g, 96.97 mmol, 21.98% yield) was obtained as a white solid and there was 20 g crude in hand.

Step 2. To a solution of compound 3B (13 g, 25.73 mmol, 1 eq.) in diethylene glycol dimethyl ether (250 mL) was added P₂S₅(9.72 g, 43.74 mmol, 4.65 mL, 1.7 eq.) and NaHCO₃ (7.35 g, 87.47 mmol, 3.40 mL, 3.4 eq.). The mixture was stirred at 130° C. for 12 hr. LCMS (ET28998-986-P1A1) showed the desired mass was detected. The reaction mixture was diluted with H₂O 100 mL and extracted with EtOAc 150 mL (50 mL*3). The combined organic layers were washed with sat. brine 100 mL, dried over Na₂SO₄, filtered and concentrated under reduced pressure to give a residue. The crude was combined, then was triturated with DCM 100 mL*3 at 25° C. for 30 min. Compound 4B (11 g, 15.61 mmol, 41.74% yield, 74% purity) was obtained as a yellow solid. LCMS (M−H⁺): 519.0.

Step 3. To a solution of compound 4B (9.9 g, 18.99 mmol, 1 eq.) in METHANOL (100 mL) was added NaOMe (3.08 g, 56.97 mmol, 3 eq.). The mixture was stirred at 20° C. for 2 hr. LCMS (ET28998-996-P1A1) showed the desired mass was detected. The reaction mixture was quenched by addition NH₄Cl 3 g, and then reduced pressure to give a residue. The crude product was purified by reversed-phase HPLC (800 g Agela C18, H2O/MeOH, 120 ml/min, 5-35% 30 min; 35% 30 min). Compound 5 (3 g, crude) was obtained as a yellow solid. LCMS (M−H⁺): 243.3.

Step 4. Batch 1: To a solution of compound 5 (1 g, 4.09 mmol, 1 eq.) in Py (10 mL) was added DMTCl (1.66 g, 4.91 mmol, 1.2 eq.). The mixture was stirred at 20° C. for 2 hr. LCMS (ET28998-1007-P1A1) showed the desired mass was detected. The reaction mixture was quenched by addition methanol 5 mL, and then concentrated under reduced pressure to give a residue. The residue was purified by column chromatography (SiO₂, Petroleum ether/Ethyl acetate=1/0 to 0/1). TLC (Petroleum ether:Ethyl acetate=1:1, Rf=0.4). 3-((2R, 4S, 5R)-5-((bis(4-methoxyphenyl)(phenyl)methoxy)methyl)-4-hydroxytetrahydrofuran-2-yl)-4-thioxo-3, 4-dihydropyrimidin-2(1H)-one (WV-NU-237) (1.12 g, 1.96 mmol, 47.85% yield, 95.61% purity) was obtained as a yellow solid. ¹H NMR (400 MHz, DMSO-d₆) δ=11.97-11.31 (m, 1H), 7.60 (dd, J=4.1, 9.0 Hz, 1H), 7.42-7.36 (m, 2H), 7.35-7.16 (m, 8H), 6.88-6.79 (m, 4H), 6.44 (d, J=7.1 Hz, 1H), 5.09 (d, J=5.6 Hz, 1H), 4.25 (br dd, J=6.1, 8.0 Hz, 1H), 3.92-3.82 (m, 1H), 3.72 (d, J=1.8 Hz, 6H), 3.30-3.24 (m, 1H), 3.06 (dd, J=3.2, 9.7 Hz, 1H), 2.60 (s, 1H), 2.05 (ddd, J=6.1, 9.0, 13.4 Hz, 1H); LCMS (M−H⁺): 545.2; purity: 95.61%.

### Synthesis of (2R, 3S, 4R, 5R)-2-(6-benzamido-7-benzoyl-8-oxo-7, 8-dihydro-9H-purin-9-yl)-5-((bis(4-methoxyphenyl)(phenyl)methoxy)methyl)-4-hydroxytetrahydrofuran-3-yl benzoate (WV-NU-229A)

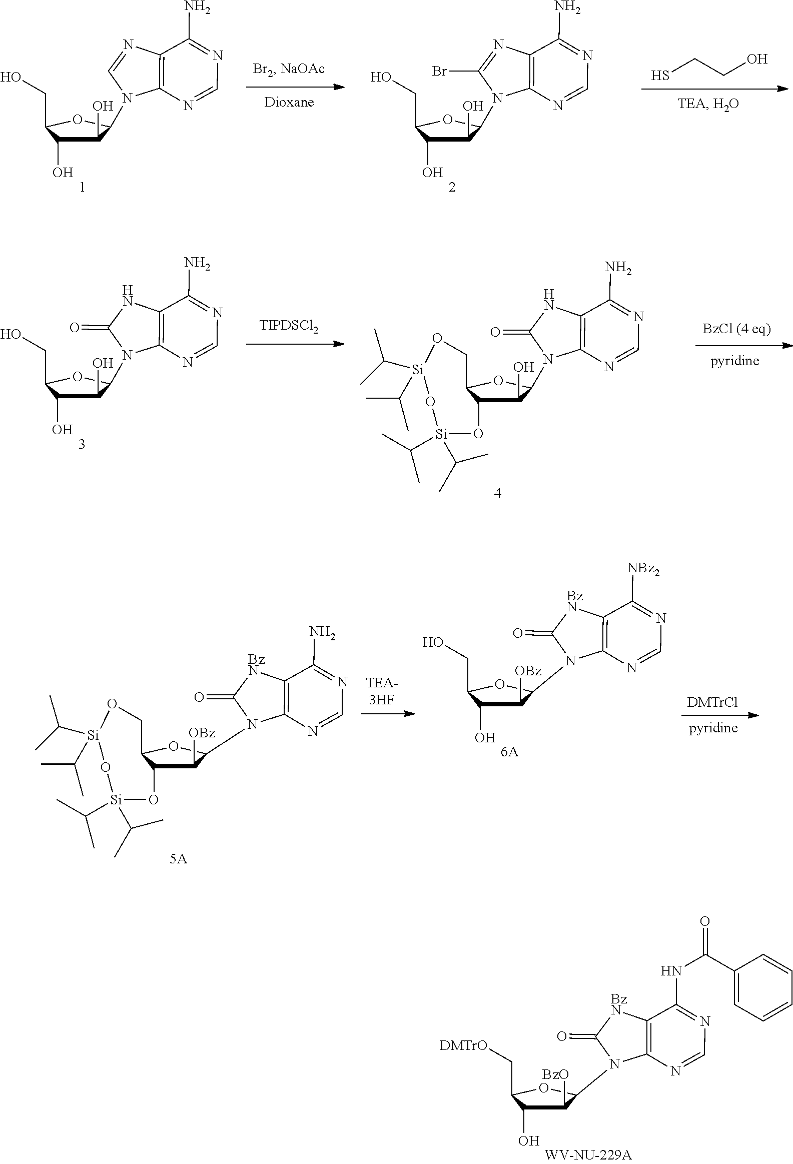

Step 1. For two batches. To a solution of compound 1 (30 g, 112.26 mmol) in H₂O (900 mL) and NaOAc (18.51 g, 225.64 mmol), AcOH (0.5 M) buffer (pH 4.7), a solution of Br₂ (21.53 g, 134.71 mmol) in Dioxane (400 mL) was added dropwise while stirring. The mixture was stirred at 25° C. for 12 hr. LCMS showed the compound 1 was consumed and the desired was found. 2 batches of reactions were combined for workup/purification. To the mixture conc.Na₂S₂O₅ was added until the red color vanished. The mixture was neutralized to pH 7.0 with 0.5 m NaOH. The residue was evaporated, when a white solid precipitated. The solid was filtered off, washed with cold 1, 4-dioxane (50 mL), and dried under high vacuum. Compound 2 (74 g, 95.22% yield) was obtained as a yellow solid. LCMS (M−H)⁻:346.1.

Step 2. For three batches. To a solution of compound 2 (24 g, 69.34 mmol) in water (400 mL) was added 2-MERCAPTOETHANOL (27.09 g, 346.68 mmol) and TEA (70.16 g, 693.37 mmol). The mixture was stirred at 110° C. for 4 hr. TLC indicated compound 2 was consumed completely and one new spot formed. 3 Batches of reactions were combined for work up. The reaction mixture was concentrated under reduced pressure to remove water. The residue was purified by column chromatography (SiO₂, Petroleum ether/Ethyl acetate=1/0 to 0/1). Compound 3 (34 g, 57.71% yield) was obtained as a yellow oil. LCMS (M−H+):284.1.

Step 3. For three batches. To a solution of compound 3 (11 g, 38.84 mmol) in PYRIDINE (200 mL) at 0° C. drops was added chloro-[chloro(diisopropyl)silyl]oxy-diisopropyl-silane (13.48 g, 42.72 mmol.) in N₂. And 2 hr latter, the mixture was stirred at 25° C. for 10 hr. TLC indicated compound 3 was consumed completely and many new spots formed. The reaction mixture was diluted with water (300 ml) and extracted with Ethyl acetate 900 mL (300 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0/1). Compound 4 (35 g, 66.57 mmol, 57.14% yield) was obtained as a yellow oil. ¹HNMR (400 MHz, DMSO-d6) δ=10.26 (s, 1H), 8.57 (dd, J=1.6, 5.8 Hz, 1H), 7.94-7.84 (m, 1H), 7.49-7.31 (m, 1H), 6.49-6.35 (m, 2H), 6.06-5.90 (m, 1H), 4.85-4.78 (m, 1H), 4.73-4.63 (m, 1H), 4.25-4.12 (m, 1H), 3.86-3.74 (m, 1H), 3.67 (dt, J=2.8, 7.7 Hz, 1H), 1.98 (s, 4H), 0.98-0.91 (m, 25H); LCMS (M−H+):897.3, purity: 99.46%.

Step 4. To a solution of compound 4 (14 g, 26.63 mmol) in PYRIDINE (140 mL) was added BzCl (14.97 g, 106.52 mmol). The mixture was stirred at 25° C. for 12 hr. LCMS showed compound 4 was consumed completely and desired mass was detected. The reaction mixture was concentrated under reduced pressure to remove pyridine. The residue was purified by column chromatography (SiO₂, Petroleum ether/Ethyl acetate=1/0 to 0/1). Compound 5A (10 g, 39.86% yield) was obtained as a yellow solid. LCMS (M−H⁺):942.6.

Step 5. To a solution of compound 5A (5 g, 5.31 mmol) in THF (50 mL) was added N, N-diethylethanamine;trihydrofluoride (2.57 g, 15.92 mmol.). The mixture was stirred at 25° C. for 2 hr. LCMS showed compound 5A was consumed completely and desired mass was detected. The reaction mixture was concentrated under reduced pressure to remove THF. The residue was purified by column chromatography (SiO₂, Petroleum ether/Ethyl acetate=1/0 to 0/1). Compound 6A (2.2 g, 59.25% yield) was obtained as a yellow solid. ¹H NMR (400 MHz, DMSO-d6) δ=8.58-8.55 (m, 1H), 7.85-7.69 (m, 8H), 7.65-7.50 (m, 8H), 7.46-7.32 (m, 7H), 6.56-6.53 (m, 1H), 5.81-5.76 (m, 1H), 5.67-5.61 (m, 1H), 4.78-4.66 (m, 2H), 3.93-3.82 (m, 1H), 3.78-3.70 (m, 1H), 3.68-3.58 (m, 1H). LCMS: (M−H)⁻: 700.3.

Step 6. To a solution of compound 6A (2.7 g, 3.86 mmol) in DCM (30 mL) was added DMTCl (1.44 g), TEA (780.98 mg, 7.72 mmol). The mixture was stirred at 25° C. for 3 hr. TLC indicated compound 6A was consumed completely and many new spot formed. The reaction mixture was diluted with water 200 mL and extracted with Ethyl acetate 900 mL (300 mL *3). The combined organic layers were dried over Na₂SO₄, filtered and concentrated under reduced pressure to give a residue. The residue was purified by column chromatography (SiO₂, Petroleum ether/Ethyl acetate=1/0 to 0/1). WV-NU-229A (2.2 g, crude) was obtained as a yellow oil. The residue was purified by column chromatography (SiO₂, Dichloromethane: Methanol=1:0 to 0:1). WV-NU-229A (3.8 g, 4.23 mmol, 80.85% yield) was obtained as a yellow solid. ¹H NMR (400 MHz, DMSO-d6) δ=8.39-8.36 (m, 1H), 7.76 (br s, 4H), 7.69-7.62 (m. 2H), 7.59-7.51 (m, 6H), 7.37 (br d, J=7.0 Hz, 6H), 7.26-7.20 (m, 4H), 7.18-7.12 (m, 2H), 7.10-7.05 (m, 1H), 6.86-6.78 (m, 4H), 6.61-6.59 (m, 1H), 5.82-5.78 (m, 1H), 5.70-5.65 (m, 1H), 4.68-4.61 (m, 1H), 4.14-4.08 (m, 1H), 3.73-3.68 (m, 6H), 3.54-3.43 (m, 1H), 3.23-3.15 (m, 1H). LCMS (M−H+):897.3, purity: 99.46%.

### Synthesis of N-(9-((2R, 4S, 5R)-5-((bis(4-methoxyphenyl)(phenyl)methoxy)methyl)-4-hydroxytetrahydrofuran-2-yl)-8-thioxo-8 9-dihydro-7H-purin-6-yl)henzamide (WV-NU1-749)

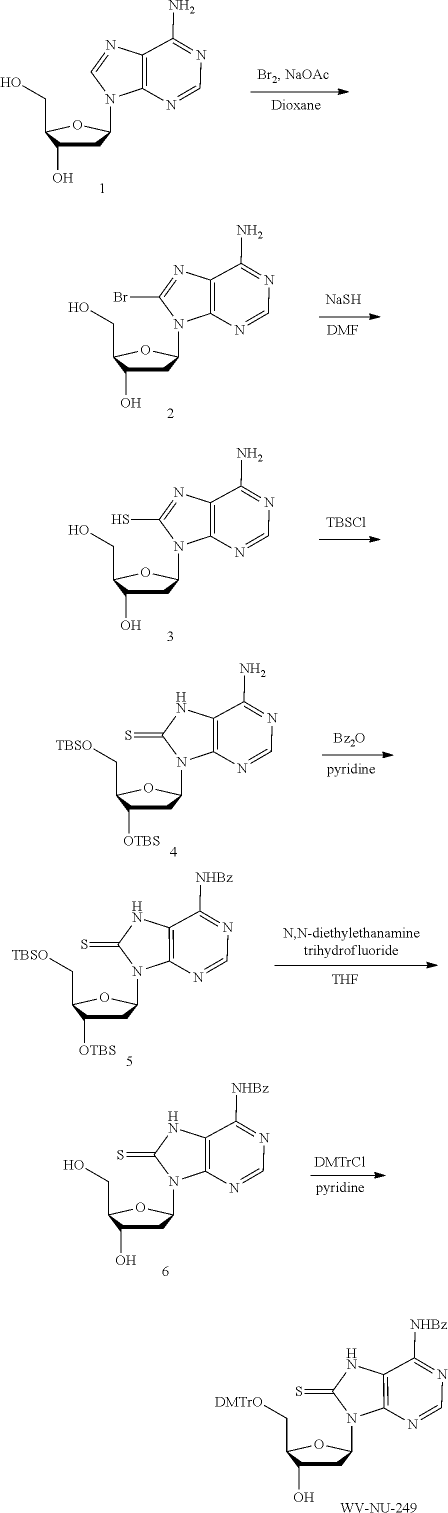

Step 1. For three batches. To a 3 L reactor, charged NaOAc (20.24 g, 246.77 mmol) water (1250 mL), and Dioxane (500 mL), adjust the pH to 4.7. And compound 1 (31 g, 123.39 mmol) was added. The Br₂ (23.67 g, 148.10 mmol) was diluted in Dioxane (500 mL) and added to the reaction mixture later. And then the mixture was stirred at 20° C. for 5 hr under N₂ atmosphere. LC-MS showed compound 1 was consumed completely and one main peak with desired mass was detected. Three reactions were combined for workup. The reaction mixture was quenched by addition sodium metabisulfite until the red was gone, and then adjust the pH to 7. Concentrated under reduced pressure until solid precipitates. The reaction mixture was filtered. The filter cake was dried by reduced pressure to give a yellow solid. Compound 2 (100 g, 81.83% yield) was obtained as a yellow solid. LCMS (M+H)⁺: 157.2, purity: 90.30%.

Step 2. For two batches. A mixture of compound 2 (10 g, 30.29 mmol.), NaSH (5.09 g, 90.87 mmol, 3 eq.) in DMF (100 mL) was degassed and purged with N₂ for 3 times, and then the mixture was stirred at 20° C. for 12 hr under N₂ atmosphere. TLC showed the product was detected. Two reactions were combined for workup. Adjust the pH to 6. The reaction mixture was concentrated under reduced pressure to give a crude product. The crude product 3 (17 g, crude) as yellow oil was used into the next step without further purification.

Step 3. A mixture of compound 3 (17 g, 60.01 mmol), TBSCl (27.13 g, 180.02 mmol), imidazole (20.43 g, 300.03 mmol) in DCM (100 mL) was degassed and purged with N₂ for 3 times, and then the mixture was stirred at 20° C. for 3 hr under N₂ atmosphere. LCMS showed the product was detected. The reaction was quenched by the addition of 3000 mL of water. Extracted three times with 5000 mL of CH₂Cl₂, the organic phase was dried over Na₂SO₄ and concentrated under reduced pressure. The residue was purified by column chromatography (SiO₂, Petroleum ether:Ethyl acetate=10:1 to 0:1). Compound 4 (24.4 g, 79.45% yield) was obtained as a white solid. ¹HNMR (400 MHz, DMSO-d₆) δ=12.49 (s, 1H), 3.90 (dd, J=6.4, 10.8 Hz, 1H), 3.81-3.74 (m, 1H), 3.81-3.74 (m, 1H), 3.61 (dd, J=5.3, 10.8 Hz, 1H), 2.88 (s, 3H), 2.75-2.70 (m, 2H), 2.09 (br s, 1H), 0.89 (s, 1OH), 0.81 (s, 9H), 0.13-0.07 (m, 1H), 0.10 (d, J=1.3 Hz, 5H), 0.04 (d, J=13.8 Hz, 6H); LCMS (M+H)⁺: 512.2, purity: 91.34%.

Step 4. To a solution of compound 4 (7.3 g, 14.26 mmol) in pyridine (80 mL) was added DMAP (1.74 g, 14.26 mmol.), benzoyl benzoate (4.84 g, 21.39 mmol). The reaction mixture was stirred at 70° C. for 12h. TLC showed the product was detected. The mixture was diluted with H₂O (500 mL) the aqueous layer was extracted with dichloromethane (3×500 mL), the combined organic phases dried over Na₂SO₄, filtered and evaporated to dryness. The residue was purified by column chromatography (SiO₂, Petroleum ether:Ethyl acetate=10:1 to 5:1). Compound 5 (7 g, 11.36 mmol, 87.50% yield) was obtained as a white solid.

Step 5. To a solution of compound 5 (7 g, 11.36 mmol.) in THF (80 mL) was added HF: TEA (10.99 g, 68.19 mmol). The mixture was stirred at 25° C. for 12 hr. TLC showed the product was detected. Filtered, the residue was washed with Methanol and concentrated under reduced pressure to give the product. Without purification used for next step. Compound 6 (2.1 g, crude) was obtained as a white solid. LCMS (M+H⁺): 386.2.

Step 6. To a solution of compound 6 (2.1 g, 5.42 mmol) in pyridine (20 mL) was added DMAP (331.12 mg, 2.71 mmol) and DMTrCl. The reaction mixture was stirred at 25° C. for 3h. LCMS showed the product was detected. Filtered and concentrated under reduced pressure to give a residue. The residue was purified by column chromatography (SiO₂, Petroleum ether:Ethyl acetate=10:1 to 0:1). 3.8 g product was obtained. ¹HNMR (400 MHz, DMSO-d₆) δ=12.78-12.61 (m, 1H), 11.41 (s, 1H), 8.31 (s, 1H), 8.09-8.02 (m, 2H), 7.69-7.63 (m, 1H), 7.60-7.49 (m, 2H), 7.36 (d, J=7.2 Hz, 2H), 7.25-7.18 (m, 6H), 7.18-7.13 (m, 1H), 6.89-6.75 (m, 4H), 6.92-6.74 (m, 1H), 5.36 (d, J=4.6 Hz, 1H), 4.69-4.50 (m, 1H), 4.06-3.99 (m, 1H), 4.00-3.99 (m, 1H), 4.06-3.99 (m, 1H), 3.71 (d, J=5.5 Hz, 6H), 3.32-3.25 (m, 1H), 3.24-3.11 (m, 2H), 2.24-2.15 (m, 1H); LCMS (M+H+): 688.3, purity: 99.57%.

### Synthesis of 3-((2R, 3R 4R, 5R)-5-((bis(4-methoxyphenyl)(phenyl)methoxy)methyl)-3-((tert-butyldimethylsilyl)oxy)-4-hydroxvtetrahydrofuran-2-yl)pyrimidine-2, 4(1H, 3H)-dione (WV-NU-222) and 3-((2R, 3R, 4S, 5R)-5-((bis(4-methoxyphenyl)(phenyl)methoxy)methyl)-4-((tert-butyldimethylsilyl)oxy)-3-hydroxytetrahydrofuran-2-yl)pyrimidine-2, 4(1H, 3H)-dione (WV-NU-222A)

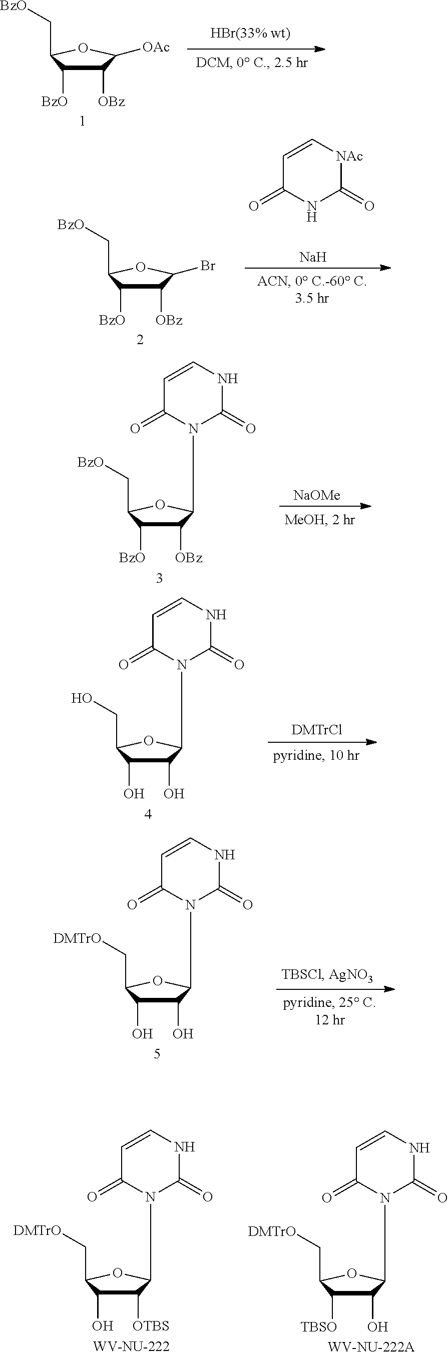

Step 1. To a solution of compound 1 (200 g, 396.44 mmol) in DCM (2000 mL) was added hydrogen bromide (388.81 g, 1.59 mol, 33% purity) at 0° C. in N₂. The mixture was stirred at 0° C. for 2 hr. TLC indicated compound 1 was consumed completely and two new spots formed. The mixture of reaction was poured into 500 mL of ice water, and the organic phase was separated, washed with water (5×300 mL), dried over Na₂SO₄, and evaporated under reduced pressure. And then the crude product to remove remaining acetic acid and dried in vacuous. Compound 2 (208 g, crude) was obtained as a yellow oil.

Step 2. For four batches: To a solution of 1-acetylpyrimidine-2, 4(1H, 3H)-dione (22.88 g, 148.47 mmol) in ACN (2000 mL) was added NaH (7.92 g, 197.97 mmol, 60% purity) at 0° C. in N₂ for 30 min. And then added compound 2 (52 g, 98.98 mmol) for 30 min at 25° C., latter 1 hr heat up to 40° C. And then the mixture was stirred at 60° C. for 2 hr. TLC indicated compound 2 was consumed completely and four new spots formed. Filtration to remove solids, the organic phase vacuum concentration to obtain coarse product. The residue was purified by column chromatography (SiO₂, Petroleum ether:Ethyl acetate=1:0 to 0:1). Compound 3 (76 g, 34.55% yield) was obtained as a yellow solid. ¹HNMR (400 MHz, DMSO-d6) δ=11.40 (br s, 1H), 7.98 (dd, J=1.1, 8.3 Hz, 2H), 7.91 (dd, J=1.2, 8.3 Hz, 2H), 7.82 (dd, J=1.3, 8.3 Hz, 2H), 7.68-7.57 (m, 3H), 7.54 (br d, J=7.6 Hz, 1H), 7.50-7.42 (m, 4H), 7.41-7.35 (m, 2H), 6.50 (s, 1H), 6.11-6.05 (m, 2H), 5.68 (d, J=7.6 Hz, 1H), 4.71-4.61 (m, 2H), 4.55-4.48 (m, 1H).

Step 3. To a solution of compound 3 (40 g, 71.88 mmol) was in MeOH (400 mL) and then added NaOMe (11.65 g, 215.63 mmol). The mixture was stirred at 25° C. for 3 hr. TLC indicated compound 3 was consumed completely and two new spots formed. The reaction was added NH₄Cl (11 g) and stirred at room temperature for 5 min, and then vacuum concentration. The crude is dissolved in MeOH (200 ml) and then pour into the EtOAc (1000 ml*3) at 25° C. stir at 20 min. And then filtered, got to the white solid to reduced pressure concentration drying. Compound 4 (15 g, 85.46% yield) was obtained as a white solid. ¹HNMR (400 MHz, DMSO-d6) δ=7.43 (d, J=7.4 Hz, 1H), 6.07 (d, J=3.6 Hz, 1H), 5.50 (d, J=7.4 Hz, 1H), 4.47 (dd, J=3.9, 6.1 Hz, 1H), 4.09 (t, J=6.3 Hz, 1H), 3.68 (dt, J=3.3, 6.2 Hz, 1H), 3.58 (dd, J=3.2, 11.7 Hz, 1H), 3.40 (br dd, J=6.1, 11.8 Hz, 1H); LCMS (M+H⁺): 267.1, purity: 96.15%.

Step 4. To a solution of compound 4 (14 g, 57.33 mmol) in Py (1500 mL) was added DMT-C1 (19.43 g, 57.33 mmol) in N₂. The mixture was stirred at 25° C. for 10 hr. TLC indicated compound 4 was consumed completely and two new spots formed. The reaction mixture was quenched by addition MeOH 50 mL, and then filtered and concentrated under reduced pressure to give a residue. The residue was purified by column chromatography (SiO₂, Petroleum ether:Ethyl acetate=1:1 to 0:1 to Ethyl acetate: MeOH=1:1, 5% TEA). Compound 5 (23 g, 42.08 mmol, 76.67% yield) was obtained as a red solid. ¹HNMR (400 MHz, DMSO-d6) δ=7.51-7.36 (m, 4H), 7.34-7.17 (m, 7H), 6.91-6.82 (m, 4H), 6.13 (d, J=2.1 Hz, 1H), 5.59 (d, J=7.6 Hz, 1H), 5.08 (br d, J=4.8 Hz, 1H), 4.86 (br d, J=7.4 Hz, 1H), 4.35 (br t, J=6.3 Hz, 1H), 4.16 (q, J =7.2 Hz, 1H), 3.87 (dt, J=3.9, 6.9 Hz, 1H), 3.74 (d, J=1.5 Hz, 6H), 3.20-3.06 (m, 2H); LCMS (M−H⁺): 545.4.

Step 5. To a solution of compound 5 (22 g, 40.25 mmol) and Py (63.68 g, 805.03 mmol) in THF (220 mL) was added AgNO₃ (25.58 g, 150.59 mmol) and TBSCl (9.10 g, 60.38 mmol). The mixture was stirred at 25° C. for 10 hr. TLC indicated compound 5 was consumed completely and two new spots formed. The reaction was filtered to remove the insoluble and partitioned between H₂O 500 mL and extracted with EtOAc (300 mL*3). The combined organic layers were washed with brine 500 ml, dried over Na₂SO₄, filtered and concentrated under reduced pressure to give a residue. The crude product was purified by reversed-phase HPLC (column: Welch Xtimate C18 250* 100 mm #10 um; mobile phase: [water (NH₄HCO₃) -ACN]; B %: 60%-85%, 20 min). Compound WV-NU-222 (6.94 g, 36.53% yield) was obtained as a white solid, and compound WV-NU-222A (7.77 g, 40.89% yield) was obtained as a white solid.

WV-NU-222: ¹H NMR (400 MHz, DMSO-d6) δ=11.16 (br s, 1H), 7.48 (br d, J=7.5 Hz, 1H), 7.40 (br d, J=7.5 Hz, 2H), 7.32-7.17 (m, 7H), 6.92-6.80 (m, 4H), 6.11 (d, J=2.8 Hz, 1H), 5.60 (d, J=7.5 Hz, 1H), 4.61-4.51 (m, 2H), 4.19-4.10 (m, 1H), 3.87 (dt, J=3.4, 6.6 Hz, 1H), 3.73 (s, 6H), 3.20-3.06 (m, 2H), 0.83 (s, 9H), 0.00 (d, J=8.8 Hz, 6H); LCMS (M−H⁺): 659.3, purity: 94.77%.

WV-NU-222A: ¹H NMR (400 MHz, DMSO-d6) δ=11.12 (br s, 1H), 7.47 (br d, J=6.8 Hz, 1H), 7.39 (br d, J=7.5 Hz, 2H), 7.31-7.16 (m, 7H), 6.84 (dd, J=2.3, 8.9 Hz, 4H), 6.10 (d, J=2.0 Hz, 1H), 5.58 (d, J=7.6 Hz, 1H), 4.84 (d, J=6.3 Hz, 1H), 4.41-4.31 (m, 2H), 3.88 (dt, J=2.5, 6.7 Hz, 1H), 3.72 (s, 6H), 3.18 (dd, J=2.6, 10.1 Hz, 1H), 2.99 (dd, J=6.9, 10.0 Hz, 1H), 0.74 (s, 9H), −0.02-−0.15 (m, 6H); LCMS (M−H⁺): 659.3, purity: 94.89%.

### Synthesis of 1-(9-((2R, 4S, 5R)-5-((bis(4-methoxyphenyl)(phenyl)methoxy)methyl)-4-hydroxytetrahydrofuran-2-yl)-8-oxo-8, 9-dihydro-7H-purin-6-yl)-3-(naphthalen-2-yl)urea (WV-NU-244) NaHCO₃

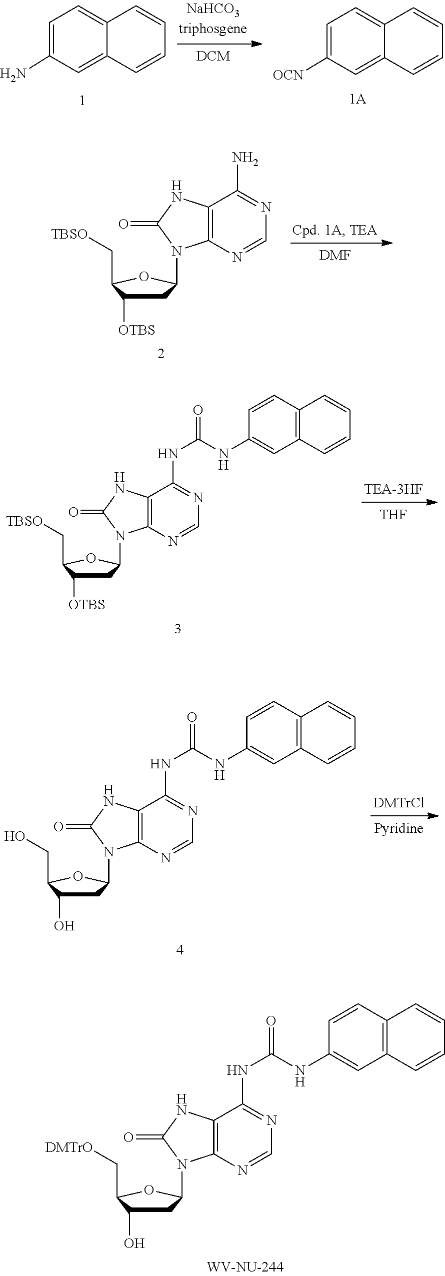

Step 1. To a mixture of compound 1 (25 g, 174.60 mmol.) in DCM (250 mL) and NaHCO₃ (250 mL) was added triphosgene (19.17 g, 64.60 mmol.) at 0° C. The mixture was stirred at 0° C. for 1.5 hr. TLC indicated compound 1 was consumed completely and one new spot formed. The organic layer was separated and dried over sodium sulfate. After filtration the organics were concentrated to an oil which solidified on standing to afford the compound. Compound IA (28 g, crude) was obtained as a brown solid.

Step 2. To a solution of compound 2 (25 g, 50.43 mmol.) in DMF (250 mL) was added compound 1A (8.53 g, 50.43 mmol.) and TEA (5.10 g, 50.43 mmol). The mixture was stirred at 80° C. for 12 hr. LCMS showed 8% of compound 2 remained. Several new peaks were shown on LCMS and 34% of desired compound 3 was detected. The reaction mixture was concentrated under reduced pressure to remove DMF. Compound 3 (33.53 g crude) was obtained as a brown oil. LCMS (M−H+): 663.4.

Step 3. To a solution of compound 3 (33.53 g, 50.43 mmol) in THF (350 mL) was added TEA-3HF (81.29 g, 504.26 mmol). The mixture was stirred at 40° C. for 2 hr. LCMS showed compound 3 was consumed completely and 35% of desired compound was detected. The reaction mixture was concentrated under reduced pressure to remove THF. The compound was filtered and got a residue and filtrate. The crude product was used into the next step without further purification. Compound 4 (22 g crude) was obtained as a yellow oil. LCMS (M−H⁺):435.2.

Step 4. To a solution of compound 4 (11 g, 25.21 mmol, 1 eq.) in PYRIDINE (100 mL) and DMSO (100 mL) was added DMTCl (8.54 g, 25.21 mmol, 1 eq.). The mixture was stirred at 40° C. for 12 hr. LCMS (ET52262-46-P1A3) showed 7% of compound 4 remained. Several new peaks were shown on LCMS and 23% of desired compound WV-NU-244 was detected. The reaction mixture was concentrated under reduced pressure to remove PYRIDINE. The crude product was purified by reversed-phase HPLC (0.1% NH₃·H₂O). Compound WV-NU-244 (4.14 g, 21.57% yield) was obtained as a little pink solid. ¹H NMR (400 MHz, DMSO-d₆) δ=10.89 (s, 1H), 10.38 (s, 1H), 9.63 (s, 1H), 8.29 (s, 1H), 8.23 (d, J=1.3 Hz, 1H), 7.89 (dd, J=8.7, 19.8 Hz, 3H), 7.62 (dd, J=2.0, 8.8 Hz, 1H), 7.51-7.39 (m, 2H), 7.34 (d, J=7.3 Hz, 2H), 7.24-7.18 (m, 7H), 6.83-6.73 (m, 4H), 6.22 (t, J=6.7 Hz, 1H), 5.28 (d, J=4.6 Hz, 1H), 4.53-4.46 (m, 1H), 3.97-3.92 (m, 1H), 3.70 (d, J=9.3 Hz, 6H), 3.22-3.11 (m, 2H), 3.04 (td, J=6.4, 12.9 Hz, 1H), 2.19-2.09 (m, 1H); LCMS (M−H+):737.4.

### Synthesis of 9-((2R, 4S, 5R)-5-((bis(4-methoxyphenyl)(phenyl)methoxy)methyl)-4-hydroxytetrahydrofuran-2-yl)-9H-purin-6-yl diphenylcarbamate (WV-NU-259) and 9-((2R, 4S, 5R)-5-((bis(4-methoxyphenyl)(phenyl)methoxy)methyl)-4-hydroxytetrahydrofuran-2-yl)-6-oxo-N, N-diphenyl-6, 9-dihydro-1H-purine-1-carboxamide (WV-NU-259A)

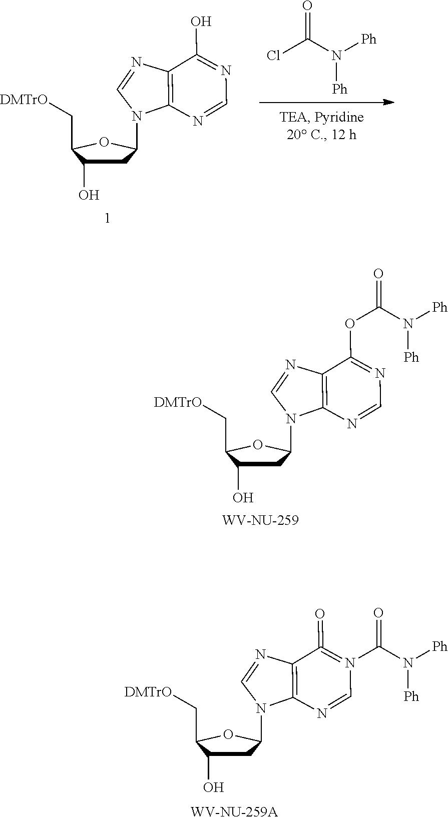

To a solution of compound 1 (40 g, 72.13 mmol) in pyridine (600 mL) was added TEA (72.98 g, 721.25 mmol) and N, N-diphenylcarbamoyl chloride (23.39 g, 100.98 mmol). The mixture was stirred at 25° C. for 4 hr. TLC indicated compound 1 was consumed completely and two new spot formed. The reaction was (clean) according to TLC. The reaction mixture was partitioned between H₂O 2000 mL and EtOAc 1000 mL. The organic phase was separated, washed with brine 500 mL dried over Na₂SO₄, filtered and concentrated under reduced pressure to give a residue. The residue was purified by column chromatography (SiO₂, Petroleum ether/Ethyl acetate=10/1 to 0/1). Compound WV-NU-259A (38.25 g, 70.09% yield) was obtained as a yellow solid. Compound WV-NU-259 (7.2 g, 2.24% yield) was obtained as a yellow solid.

WV-NU-259A: ¹HNMR (DMSO-d6, 400 MHz): δ=8.74-8.83 (m, 1H), 8.19 (d, J=12.1 Hz, 1H), 7.13-7.54 (m, 19H), 6.77-6.87 (m, 4H), 6.25 (t, J - 6.4 Hz, 1H), 5.38 (d, J -4.4 Hz, 1H), 4.38 (br s, 1H), 3.92 (br d, J=11.6 Hz, 1H), 3.73 (s, 6H), 3.01-3.20 (m, 2H), 2.59-2.71 (m, 1H), 2.33 ppm (br s, 1H); LCMS (M−H⁺): 748.

WV-NU-259: ¹HNMR (DMSO-d6, 400 MHz): δ=8.74 (s, 1H), 8.65 (s, 1H), 7.39-7.56 (m, 8H), 7.24-7.38 (m, 4H), 7.09-7.23 (m, 7H), 6.77 (dd, J=15.6, 8.8 Hz, 4H), 6.50 (t, J=6.2 Hz, 1H), 5.43 (d, J=4.4 Hz, 1H), 4.44-4.55 (m, 1H), 3.98-4.06 (m, 1H), 3.66 (d, J=10.1 Hz, 6H), 3.13-3.26 (m, 2H), 2.89-2.98 (m, 1H), 2.40 ppm (dt, J - 12.7, 6.4 Hz, 1H); LCMS (M−H⁺): 748.

### Synthesis of 3-((2R, 4S, 5R)-5-((bis(4-methoxyphenyl)(phenyl)methoxy)methyl)-4-hydroxytetrahydrofuran-2-yl)-5-ethynylpyrimidine-2, 4(1H, 3H)-dione_(WV-NU-256A)

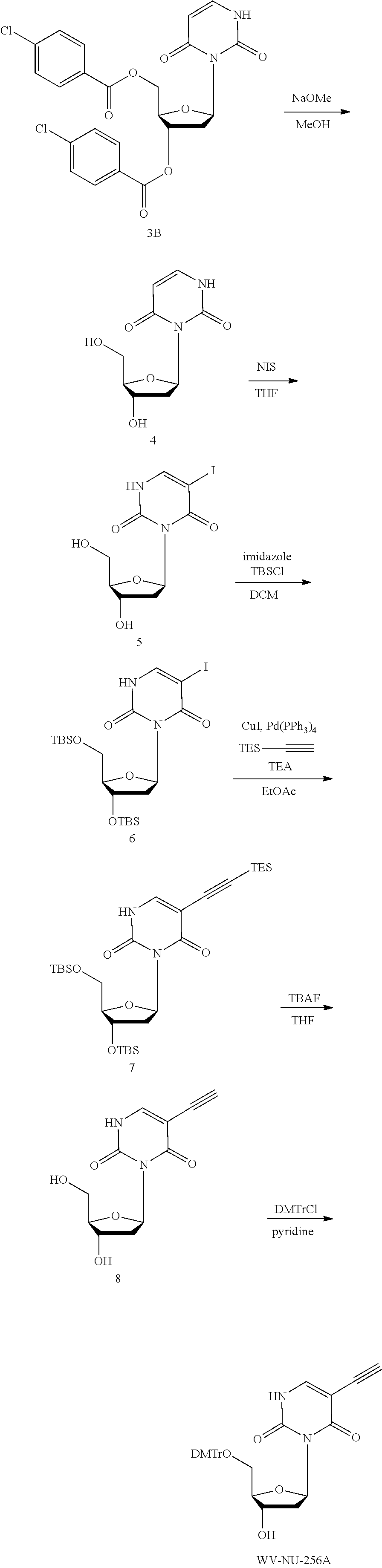

Step 1. To a solution of compound 3B (26 g, 51.45 mmol) in MeOH (400 mL) was added NaOMe (7.51 g, 138.93 mmol). The mixture was stirred at 25° C. for 1 hr. TLC indicated compound 3B was consumed completely and one new spot formed. The reaction was clean according to TLC. The mixture was filtered and concentrated under reduced pressure to give a residue. The residue was purified by column chromatography (SiO₂, Ethyl acetate: Methanol=100/1 to 1/1). Compound 4 (11 g, 93.68% yield) was obtained as a white solid.

Step 2. To a solution of compound 4 (11 g, 48.20 mmol) in THF (300 mL) was added NIS (27.11 g, 120.51 mmol). The mixture was stirred at 25° C. for 12 hr. TLC indicated compound 4 was consumed completely and one new spot formed. The reaction was clean according to TLC. The mixture was concentrated in vacuum. The residue was purified by column chromatography (SiO₂, Ethyl acetate: Methanol=100/1 to 0/1). Compound 5 (13.5 g, 79.09% yield) was obtained as a yellow solid. ¹H NMR (DMSO-d₆, 400 MHz): δ=11.42 (br d, J=5.9 Hz, 1H), 7.93 (d, J=6.0 Hz, 1H), 6.52 (dd, J=8.1, 6.1 Hz, 1H), 4.28 (dt, J=7.3, 4.9 Hz, 1H), 3.63-3.69 (m, 1H), 3.54-3.62 (m, 1H), 3.38-3.48 (m, 1H), 2.61-2.71 (m, 1H), 1.92 ppm (ddd, J=13.0, 8.2, 4.9 Hz, 1H).

Step 3. To a solution of compound 5 (11 g, 31.06 mmol) in DCM (300 mL) was added TBSCl (9.36 g, 62.13 mmol) and imidazole (3.17 g, 46.60 mmol).The mixture was stirred at 15° C. for 12 hr. LCMS showed compound 5 was consumed completely and one main peak with desired m/z. The mixture was concentrated under reduced pressure to give a residue. The residue was purified by column chromatography (SiO₂, Petroleum ether/Ethyl acetate=100/1 to 1/1). Compound 6 (17 g, 93.93% yield) was obtained as a white solid. ¹HNMR (CHLOROFORM-d, 400 MHz): δ=7.49 (s, 1H), 6.62 (dd, J=8.6, 5.1 Hz, 1H), 4.45 (dt, J=7.5, 5.0 Hz, 1H), 3.69-3.86 (m, 3H), 2.70 (ddd, J=13.1, 7.8, 5.1 Hz, 1H), 1.96-2.06 (m, 1H), 0.82 (d, J=3.1 Hz, 18H), 0.00 ppm (t, J=2.1 Hz, 12H); LCMS (M+Na⁺)=605.

Step 4. To a solution of compound 6 (10 g, 17.16 mmol) in EtOAc (100 mL) was added TEA (2.61 g, 25.75 mmol) and CuI (16.34 mg, 85.82umol), triethyl(ethynyl)silane (7.22 g, 51.49 mmol, 3 eq.), Pd(PPh₃)₂Cl₂ (60.24 mg, 85.82umol). The mixture was stirred at 15° C. for 12 hr. TLC indicated compound 6 was consumed completely and two new spot) formed. The reaction was clean according to TLC. The mixture was concentrated under reduced pressure to give a residue. The residue was purified by column chromatography (SiO₂, Petroleum ether/Ethyl acetate=100/1 to 0/1). Compound 7 (6.5 g, 63.65% yield) was obtained as a white solid.

Step 5. To a solution of compound 7 (6.5 g, 10.92 mmol) in THF (65 mL) was added TBAF (2 M, 21.85 mL). The mixture was stirred at 15° C. for 4 hr. TLC indicated compound 7 was consumed completely and one new spot formed. The reaction was clean according to TLC. The mixture was concentrated under reduced pressure to give a residue. The residue was purified by column chromatography (SiO₂, Petroleum ether/Ethyl acetate=100/1 to 1/1). Compound 8 (2.5 g, 90.73% yield) was obtained as a white solid.

Step 6. To a solution of compound 8 (2.5 g, 9.91 mmol) in Py (20 mL) was added DMTCl (3.36 g, 9.91 mmol). The mixture was stirred at 15° C. for 2 hr. LCMS showed compound 8 was consumed completely and one main peak with desired m/z. The reaction mixture was partitioned between H₂O 50 mL and EtOAc100 mL. The organic phase was separated, washed with brine 50 mL dried over Na₂SO₄, filtered and concentrated under reduced pressure to give a residue. The residue was purified by column chromatography (SiO₂, Petroleum ether/Ethyl acetate=100/1 to 0/1). Compound WV-NU-256A (3.3 g, 60.03% yield) was obtained as a white solid. ¹H NMR (CHLOROFORM-d, 400 MHz): δ=9.24 (br s, 1H), 7.45 (br d, J=7.4 Hz, 1H), 7.26-7.39 (m, 8H), 7.18-7.23 (m, 3H), 6.79-6.89 (m, 4H), 6.69 (dd, J=9.1, 4.1 Hz, 1H), 4.66-4.82 (m, 1H), 3.81 (d, J=10.9 Hz, 6H), 3.51 (dd, J=9.5, 6.3 Hz, 1H), 3.38 (dd, J=9.6, 5.6 Hz, 1H), 2.77-2.86 (m, 1H), 2.18-2.30 ppm (m, 1H); LCMS (M−H⁺): 553 purity: 98%.

### Synthesis of 3-((2R, 4S, 5R)-5-((bis(4-methoxyphenyl)(phenyl)methoxy)methyl)-4-hydroxytetrahydrofuran-2-yl)-5-methylpyrimidine-2, 4(1H, 3H)-dione (WV-NU-236)

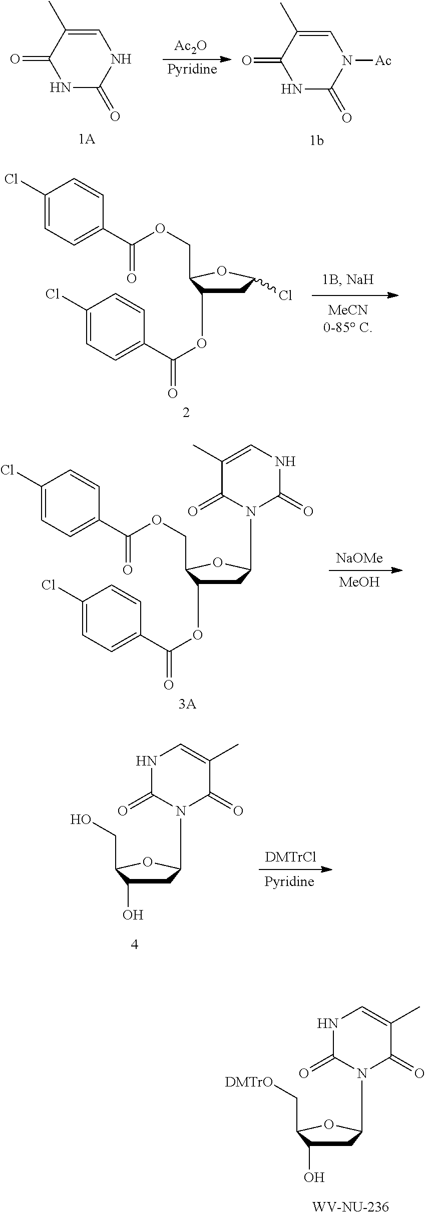

Step 1. To a solution of compound 1A (41.4 g, 328.28 mmol) in pyridine (400 mL) was added Ac₂O (201.08 g, 1.97 mol). The mixture was stirred at 120° C. for 3 hr. TLC indicated compound 1A was consumed completely and one new spot formed. The reaction was clean according to TLC. The mixture was concentrated under reduced pressure to give a residue. The crude product was triturated with Petroleum ether: Ethyl acetate=10:1 at 15° C. for 5 min. Compound 1B (46 g, 83.33% yield) was obtained as a white solid.

Step 2. A clean and dry three-neck 1 L round bottom flask charge with compound 1B (11.74 g, 69.82 mmol) and dissolved into dry MeCN (300 mL) under N₂ atmosphere. Cool the reaction mixture to 0° C. by using ice bath. Then NaH (4.19 g, 104.73 mmol, 60% purity) was added portion wise to the reaction mixture and stir for 30 min at 0° C. After Compound 2 (30 g, 69.82 mmol) was added portion wise and stir the reaction mixture for 30 min at 0-45° C. for 1 hr and at 65° C. for 2 hr. TLC show that compound 2 was consumed and new spots was formed. Then, cool the reaction mixture to 15° C. and filter through sintered funnel using what man filter paper. The filtrate was concentrated under reduced pressure to give a crude product. The residue was purified by column chromatography (SiO₂, Petroleum ether/Ethyl acetate=100/1 to 1/1). Then, the crude product was purified by re-crystallization from EtOAc (100 mL) at 15° C. Compound 3A (6 g, 16.55% yield) was obtained as a white solid.

Step 3. To a solution of compound 3A (6.5 g, 12.52 mmol) in MeOH (90 mL) was added NaOMe (1.69 g, 31.29 mmol). The mixture was stirred at 20° C. for 12 hr. LCMS showed compound 3A was consumed completely and one main peak with desired mass was detected. The reaction mixture was quenched with NH₄Cl solid (1.7 g), filtrate the solids and get filtrate. The residue was purified by column chromatography (SiO₂, Petroleum ether/Ethyl acetate=1/0 to 0/1, Dichloromethane: Methanol=1/0 to 0/1). Compound 4 (2.8 g, 92.41% yield) was obtained as a white solid. ¹H NMR (400 MHz, DMSO-d₆) δ=10.93-10.83 (m, 1H), 7.31 (s, 1H), 6.61-6.52 (m, 1H), 5.07 (d, J=4.6 Hz, 1H), 4.59-4.53 (m, 1H), 4.34-4.26 (m, 1H), 4.13-4.07 (m, 1H), 3.70-3.63 (m, 1H), 3.62-3.54 (m, 1H), 3.49-3.40 (m, 1H), 1.91 (dd, J=4.5, 8.1, 12.8 Hz, 1H), 1.75 (d, J=0.7 Hz, 3H); LCMS (M−H⁺): 241.2.

Step 4. To a solution of Compound 4 (2.8 g, 11.56 mmol) in Pyridine (70 mL) was added DMTCl (4.31 g, 12.72 mmol). The mixture was stirred at 20° C. for 12 hr. LCMS showed Compound 4 was consumed completely and one main peak with desired mass was detected. The reaction mixture was diluted with H₂O 100 mL and extracted with EtOAc 200 mL (100 mL*2). The combined organic layers were washed with saturated salt water 200 mL (100 mL*2), dried over with saturated Mg₂SO₄, filtered and concentrated under reduced pressure to give a residue. The residue was purified by column chromatography (SiO₂, Petroleum ether/Ethyl acetate=1/0 to 0/1). Compound WV-NU-236 (5 g, 75.71% yield) was obtained as a yellow foam. ¹HNMR (400 MHz, DMSO-d₆) δ=10.84 (d, J=5.7 Hz, 1H), 7.40 (d, J=7.2 Hz, 2H), 7.32 (dd, J=1.0, 5.7 Hz, 1H), 7.30-7.22 (m, 6H), 7.22-7.16 (m, 1H), 6.88-6.79 (m, 4H), 6.63 (dd, J=4.3, 9.0 Hz, 1H), 5.08-5.04 (m, 1H), 4.33-4.25 (m, 1H), 3.87-3.80 (m, 1H), 3.73 (d, J=2.2 Hz, 6H), 3.25 (t, J=8.8 Hz, 1H), 3.10-3.05 (m, 1H), 2.61 (ddd, J=4.2, 8.3, 12.9 Hz, 1H), 2.07-2.00 (m, 1H), 1.74 (s, 3H); LCMS (M−H⁺): 543.2, LCMS purity: 95.4%.

### Synthesis of 9-((2R, 4S, 5R)-5-((bis(4-methoxyphenyl)(phenyl)methoxy)methyl)-4-hydroxytetrahydrofuran-2-yl)-6-(ethylamino)-7, 9-dihydro-8H-purin- 8-one (WV-NU-245C)

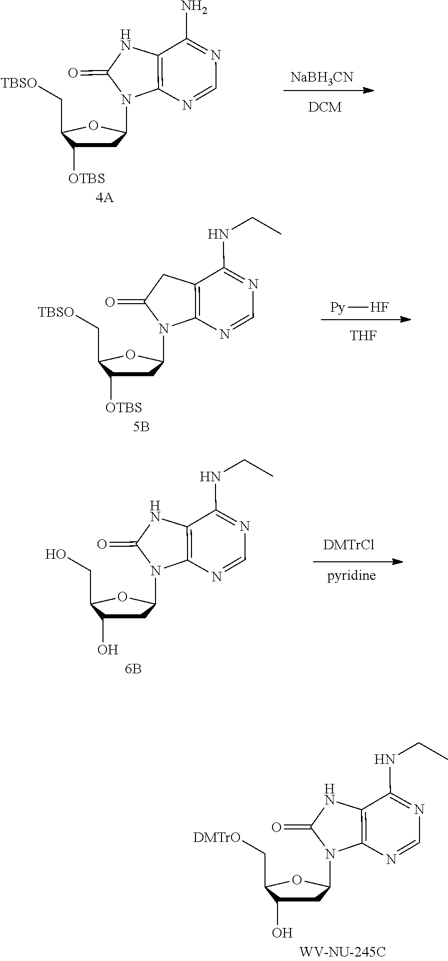

Step 1. To a solution of compound 4A (6.66 g, 151.28 mmol), tetraisopropoxytitanium (4.30 g, 15.13 mmol) in DCM (250 mL), after stirring for 2 h, the reaction become a clear solution. Then sodium triacetoxyboranuide (32.06 g, 151.28 mmol) were added. The mixture was stirred at 25° C. for 10 hr. LCMS showed compound 4A was consumed completely and one main peak with desired m/z. TLC indicated compound 4A was consumed completely and one new spot formed. The reaction was clean according to TLC. The reaction mixture was partitioned between H₂O (100 mL) and DCM (400 mL). The organic phase was separated, washed with brine 40 mL, dried over Na₂SO₄, filtered and concentrated under reduced pressure to give a residue. The residue was purified by column chromatography (SiO₂, Petroleum ether/Ethyl acetate=100/1 to 3/1). Compound 5B (10 g, 63.10% yield) was obtained as a white solid. LCMS: (M+H⁺): 524.

Step 2. To a solution of compound 5B (9 g, 17.18 mmol) in THF (100 mL) was added pyridine hydrofluoride (6.81 g, 51.54 mmol). The mixture was stirred at 37° C. for 12 hr. LCMS showed compound 5B was consumed completely and one main peak with desired m/z. The mixture was concentrated in vacuum to give a residue. The residue was purified by column chromatography (SiO₂, Dichloromethane: Methanol=100/1 to 5/1). Compound 6B (5 g, 98.55% yield) was obtained as a white solid. LCMS: (M+H⁺)=296.

Step 3. To a solution of compound 6B (5 g, 16.93 mmol) in Py (60 mL) was added DMT-C1 (5.74 g, 16.93 mmol). The mixture was stirred at 25° C. for 2 hr. TLC indicated compound 6B was consumed completely and one new spot formed. The reaction was clean according to TLC. The reaction mixture was partitioned between H₂O (100 mL) and EtOAc (300 mL). The organic phase was separated, washed with brine, dried over Na₂SO₄, filtered and concentrated under reduced pressure to give a residue. The residue was purified by column chromatography (SiO₂, Petroleum ether/Ethyl acetate=100/1 to 0/1). Compound WV-NU-245C (4.2 g, 38.18% yield) was obtained as a yellow solid. ¹H NMR (DMSO-d₆, 400 MHz): δ=10.20 (s, 1H), 7.95 (s, 1H), 7.35 (d, J=7.3 Hz, 2H), 7.13-7.26 (m, 7H), 6.75-6.89 (m, 4H), 6.44 (t, J=5.4 Hz, 1H), 6.14 (t, J=6.8 Hz, 1H), 5.22 (d, J=4.8 Hz, 1H), 4.45-4.54 (m, 1H), 3.90 (q, J=5.3 Hz, 1H), 3.68-3.77 (m, 6H), 3.42-3.50 (m, 2H), 3.14 (d, J=5.5 Hz, 2H), 3.05 (dt, J=13.0, 6.5 Hz, 1H), 2.07 ppm (ddd, J=12.8, 7.7, 4.8 Hz, 1H); LCMS: (M−H+)=596.

### Synthesis of 9-((2R, 4S, 5R)-5-((bis(4-methoxyphenyl)(phenyl)methoxy)methyl)-4-hydroxytetrahydrofuran-2-yl)-6-(isopropylamino)-7, 9-dihydro-8H-purin-8-one (WV-NU-246A)

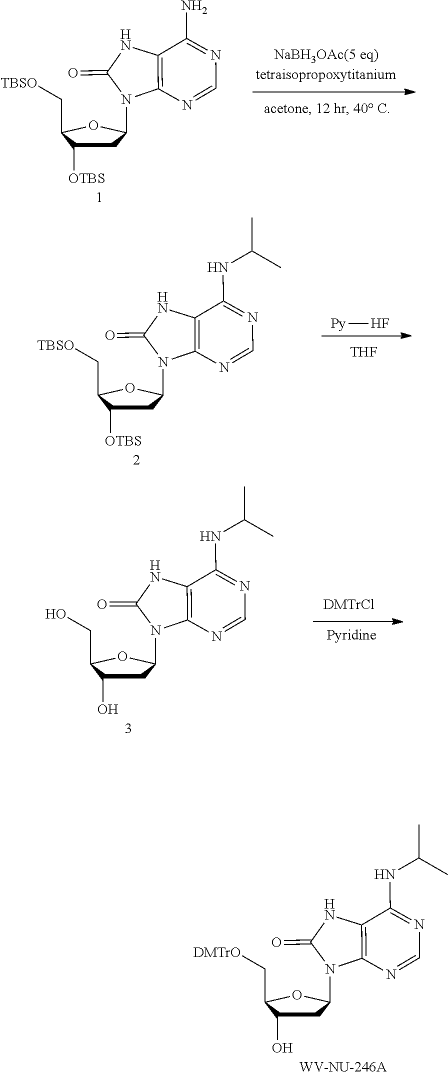

Step 1. To a solution of compound 1 (20 g, 40.34 mmol), tetraisopropoxytitanium (40.13 g, 141.20 mmol) in acetone (400 mL). After stirring at 60° C. for 4 h, the reaction become a clear solution. Then sodium triacetoxyboranuide (32.06 g, 151.28 mmol) were added. The mixture was stirred at 25° C. for 10 hr. TLC indicated compound I was consumed completely and one new spot formed. The reaction was clean according to TLC. The reaction mixture was partitioned between H₂O 500 mL and DCM 2000 mL. The organic phase was separated, washed with brine 200 mL, dried over Na₂SO₄, filtered and concentrated under reduced pressure to give a residue. The residue was purified by column chromatography (SiO₂, Petroleum ether/Ethyl acetate =100/1 to 3/1.). Compound 2 (9 g, 41.48% yield) was obtained as a white solid. ¹H NMR (CHLOROFORM-d, 400 MHz): δ=10.11 (br s, 1H), 7.95-8.08 (m, 1H), 6.22 (t, J=7.2 Hz, 1H), 5.45 (br s, 1H), 4.60 (dt, J=5.5, 2.8 Hz, 1H), 4.12-4.25 (m, 1H), 3.51-3.67 (m, 1H), 3.16-3.45 (m, 1H), 1.91-2.08 (m, 3H), 1.01-1.24 (m, 7H), 0.74-0.82 (m, 17H), -0.15-0.01 ppm (m, 11H).

Step 2. To a solution of compound 2 (9 g, 16.73 mmol) in THF (100 mL) was added pyridine;hydrofluoride (4.88 g, 41.83 mmol, 85% purity). The mixture was stirred at 37° C. for 12 hr. LCMS showed compound 5 was consumed completely and one main peak with desired m/z. The mixture was concentrated in vacuum to give a residue. The residue was purified by column chromatography (SiO₂, Dichloromethane: Methanol=100/1 to 5/1). Compound 3 (3.3 g, 63.76% yield) was obtained as a white solid. LCMS: (M+H⁺)=310.

Step 3. To a solution of compound 3 (3.3 g, 10.67 mmol) in Py (33 mL) was added DMT-Cl (3.61 g, 10.67 mmol). The mixture was stirred at 25° C. for 2 hr. LC-MS showed compound 6 was consumed completely and one main peak with desired m/z. TLC indicated compound 3 was consumed completely and one new spot formed. The reaction was clean according to TLC. The reaction mixture was partitioned between H₂O 80 mL and EtOAc 200 mL. The organic phase was separated, washed with brine 80 mL, dried over Na₂SO₄, filtered and concentrated under reduced pressure to give a residue. The residue was purified by column chromatography (SiO₂, Petroleum ether/Ethyl acetate=100/1 to 0/1). Compound WV-NU-246A (5.2 g, 74.58% yield) was obtained as a purple solid. ¹HNMR (DMSO-d₆, 400 MHz): δ=10.41 (br s, 1H), 7.94 (s, 1H), 7.25-7.39 (m, 2H), 7.09-7.33 (m, 8H), 6.74-6.86 (m, 4H), 6.56 (br s, 1H), 6.13 (t, J=6.7 Hz, 1H), 5.22 (d, J=4.5 Hz, 1H), 4.44-4.54 (m, 1H), 4.20 (dq, J=13.5, 6.6 Hz, 1H), 3.85-3.93 (m, 1H), 3.72 (d, J=4.2 Hz, 6H), 3.12-3.18 (m, 2H), 2.07 (ddd, J=12.9, 7.7, 4.7 Hz, 1H), 1.19 ppm (qd, J=7.2, 3.4 Hz, 6H); LCMS (M−H⁺)=610; purity=93%.

### Synthesis of 1-(9-((2R, 4S, 5R)-5-((bis(4-methoxyphenyl)(phenyl)methoxy)methyl)-4-hydroxytetrahydrofuran-2-yl)-8-oxo-8, 9-dihydro-7H-purin-6-yl)-3-methylurea (WV-NU-247)

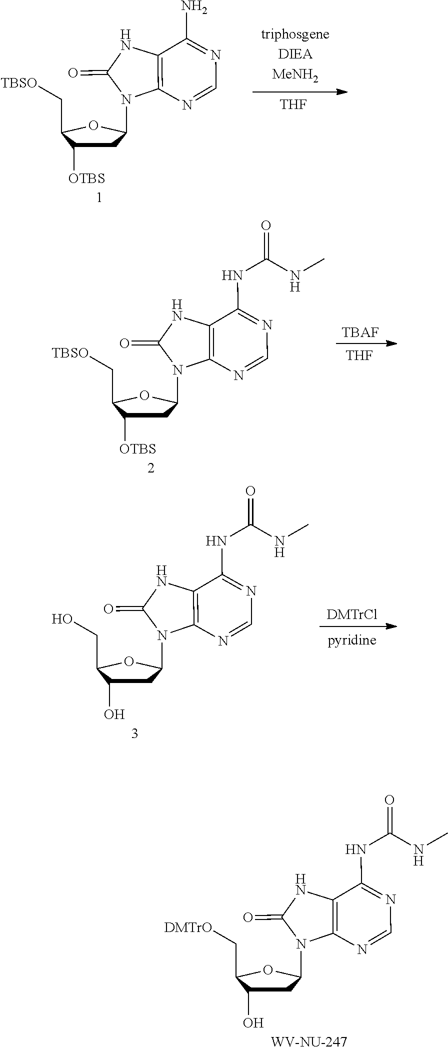

Step 1. To a solution of compound 1 (30 g, 60.51 mmol) and DIEA (19.55 g, 151.28 mmol) in THF (1500 mL) at 0° C. was added triphosgene (7.70 g, 25.96 mmol.) and the mixture was stirred for 10 min. Then MeNH₂ (2 M, 60.51 mL.) was added, the mixture was stirred for an additional 20 min, then warmed to 15° C. and stirred for 12 h under N₂. LCMS showed the desired mass was detected. Excess triphosgene was quenched with 100 mL water then the mixture was concentrated. The residue was partitioned between water 200 mL and ethyl acetate 200 mL*3, and the organic phase was collected, washed with saturated aqueous sodium bicarbonate 100 mL, and brine 100 mL. It was then dried over anhydrous Na₂SO₄, filtered, concentrated. The residue was purified by column chromatography (SiO₂, Petroleum ether/Ethyl acetate=1/0 to 0/1). Then the crude product was triturated with 20 mL methanol at 15° C. for 10 min. Compound 2 (3.5 g, 10.61% yield) was obtained as a yellow solid. LCMS (M−H⁺): 551.5.

Step 2. To a solution of compound 2 (3.5 g, 6.33 mmol.) in THF (30 mL) was added TBAF (1 M, 15.83 mL). The mixture was stirred at 15° C. for 2 hr. LCMS showed the desired mass was detected. The reaction mixture was concentrated under reduced pressure to remove solvent. The crude product was purified by reversed-phase HPLC (0.1% NH₃·H₂O). Compound 3 (1.8 g, 87.67% yield) was obtained as a white solid. LCMS (M−H⁺): 323.2.

Step 3. To a solution of compound 3 (0.9 g, 2.78 mmol.) in Py (50 mL) was added DMTCl (1.03 g, 3.05 mmol). The mixture was stirred at 15° C. for 12 hr. LCMS showed the desired mass was detected. The reaction mixture was quenched and diluted by addition sat. NaHCO₃ 50 mL, and then extracted with ethyl acetate 200 mL (100 mL*2). The combined organic layers were washed with brine 100 mL, dried over Na₂SO₄, filtered and concentrated under reduced pressure to give a residue. The residue was purified by column chromatography (SiO₂, Petroleum ether/Ethyl acetate=1/0 to 0/1, then Ethyl acetate/methanol=1/0 to 0/1, 5% TEA). Compound WV-NU-247 (1 g, 53.47% yield) was obtained as a yellow solid. ¹H NMR (400 MHz, DMSO-d₆) δ=10.28 (s, 1H), 9.32 (s, 1H), 8.33-8.24 (m, 1H), 8.15 (s, 1H), 7.34 (br d, J=7.7 Hz, 2H), 7.29-7.08 (m, 7H), 6.87-6.70 (m, 4H), 6.19 (br t, J=6.6 Hz, 1H), 5.26 (d, J=4.8 Hz, 1H), 4.54-4.42 (m, 1H), 3.93 (br d, J=5.0 Hz, 1H), 3.70 (d, J=7.0 Hz, 6H), 3.22-3.09 (m, 2H), 3.02 (td, J=6.4, 13.0 Hz, 1H), 2.78 (br d, J=4.4 Hz, 3H), 2.19-2.06 (m, 1H); LCMS (M−H⁻): 625.3; purity: 93.00%.

### Synthesis of 7-((2R, 4S, 5R)-5-((bis(4-methoxyphenyl)(phenyl)methoxy)methyl)-4-hydroxytetrahydrofuran-2-yl)-3, 7-dihydro-4H-pyrrolo[2, 3-d]pyrimidin-4-one (WV-NU-261)

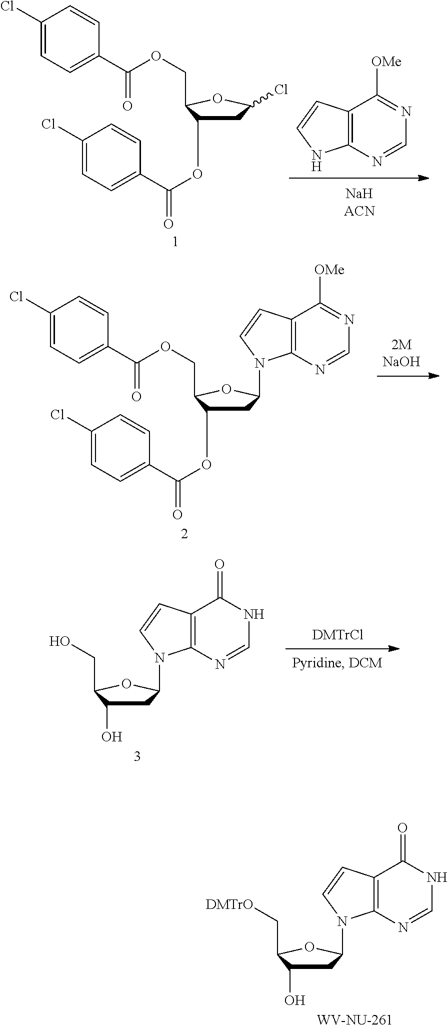

Step 1. To a solution of 4-methoxy-7H-pyrrolo[2, 3-d]pyrimidine (4.5 g, 30.17 mmol) in ACN (200 mL) was added NaH (1.81 g, 45.26 mmol, 60% purity) at 0° C. and stirred at 0° C. for 30 min, then added compound 1 (19.45 g, 45.26 mmol) to the above reaction mixture and stirred at 65° C. for 4h. TLC showed the product was detected. Filtered and concentrated under reduced pressure to give a residue. The residue was purified by column chromatography (SiO₂, Petroleum ether:Ethyl acetate=10:1 to 3:1). Compound 2 (11 g, 78.57% yield) was obtained as a white solid.

Step 2. To a solution of compound 2 (10 g, 18.44 mmol) in MeOH (50 mL) was added NaOH (2 M, 92.19 mL). The reaction mixture was stirred at 90° C. for 12h. LCMS showed the product was detected. Filtered and concentrated under reduced pressure to give a residue. The residue was purified by column chromatography (SiO₂, Dichloromethane:Methanol=60:1 to 0:1). Compound 3 (4.2 g, 90.67% yield) was obtained as a white solid. ¹H NMR (400 MHz, DMSO-d₆) δ=11.98 (br s, 1H), 7.91 (s, 1H), 7.36 (d, J=3.5 Hz, 1H), 6.52 (d, J=3.6 Hz, 1H), 6.50-6.45 (m, 1H), 5.59-4.74 (m, 2H), 4.45-4.23 (m, 1H), 3.91-3.77 (m, 1H), 3.59-3.46 (m, 2H), 2.48-2.39 (m, 1H), 2.24-2.15 (m, 1H). LCMS (M+H⁺): 250.2.

Step 3. To a solution of compound 3 (3.5 g, 13.93 mmol) in pyridine (200 mL) was added DMTCl (5.19 g, 15.32 mmol). TLC showed the product was detected. A few drops of Methanol 5 ml was added to hydrolyze any unreacted DMTrCl and the mixture was stirred for 10 minutes and concentrated under reduced pressure to give a residue. The residue was purified by column chromatography (SiO₂, Petroleum ether:Ethyl acetate=10:1 to 0:1). WV-NU-261 (3.2 g, 40.11% yield) was obtained as a yellow solid. ¹H NMR (400 MHz, DMSO-d₆) δ=11.99 (br s, 1H), 7.91 (s, 1H), 7.37 (d, J=7.4 Hz, 2H), 7.30-7.20 (m, 7H), 7.19-7.17 (m, 1H), 6.91-6.80 (m, 4H), 6.52-6.47 (m, 2H), 4.44-4.31 (m, 1H), 3.98-3.88 (m, 1H), 3.73 (s, 6H), 3.15 (br d, J=4.6 Hz, 2H), 2.58-2.44 (m, 2H), 2.27 (ddd, J=4.1, 6.2, 13.1 Hz, 1H); LCMS (M−H+): 552.3, purity: 96.67%.

### Synthesis of 3-((2R, 4S, 5R)-5-((bis(4-methoxyphenyl)(phenyl)methoxy)methyl)-4-hydroxytetrahydrofuran-2-yl)-5-(prop-1-yn-1-yl)pyrimidine-2, 4(1H, 3H)-dione (WV-NU-260)

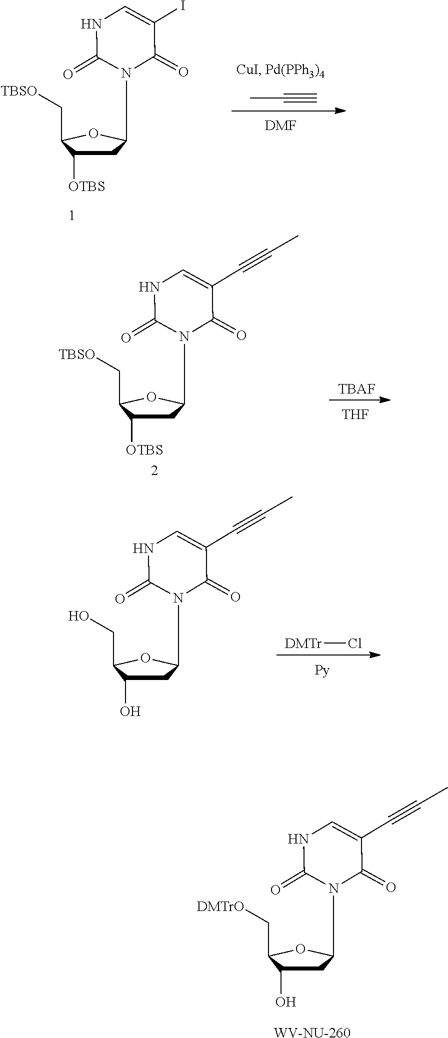

Step 1. To a solution of compound 1 (2.9 g, 4.98 mmol) in EtOAc (30 mL) was added TEA (579.23 mg, 5.72 mmol), CuI(4.74 mg, 24.89 umol) and Pd(PPh₃)₂Cl₂ (17.47 mg, 24.89 umol) prop-1-yne (2 M, 14.93 mL). The mixture was stirred at 25° C. for 12 hr. LCMS showed Reactant 1 was consumed completely and one main peak with desired m/z. The mixture was concentrated under reduced pressure to give a residue. The crude product was purified by reversed-phase HPLC (column: Welch Xtimate C18 250*70 mm #10 um; mobile phase: [water (NH₄HCO₃)-ACN]; B %: 70%-95%, 20 min). Compound 2 (0.5 g, 20.30% yield) was obtained as a white solid. LCMS (M+Na⁺): 517.

Step 2. To a solution of compound 2 (1.5 g, 3.03 mmol) in THF (15 mL) was added TBAF (1 M, 9.10 mL).The mixture was stirred at 15° C. for 2 hr. LCMS showed compound 2 was consumed completely and one main peak with desired m/z. The mixture was concentrated under reduced pressure to give a residue. The residue was purified by column chromatography (SiO₂, Dichloromethane: Methanol=100/1 to 5/1). Compound 3 (0.807 g, crude) was obtained as a white solid. LC-MS (M+Na⁺): 289.

Step 3. To a solution of compound 3 (0.7 g, 2.63 mmol) in Py (10 mL) was added DMT-Cl (890.81 mg, 2.63 mmol). The mixture was stirred at 15° C. for 2 hr. LCMS showed compound 3 was consumed completely and one main peak with desired m/z. The reaction mixture was partitioned between EtOAc 100 mL and H₂O 30 mL. The organic phase was separated, washed with brine 30 mL, dried over Na₂SO₄, filtered and concentrated under reduced pressure to give a residue. The residue was purified by column chromatography (SiO₂, Petroleum ether/Ethyl acetate=100/1 to 0/1). Compound WV-NU-260 (1 g, 62.88% yield) was obtained as a white solid. ¹HNMR (DMSO-d₆, 400 MHz): δ=11.43 (br s, 1H), 7.78 (s, 1H), 7.44 (d, J=7.4 Hz, 2H), 7.22-7.36 (m, 7H), 6.90 (t, J=8.9 Hz, 4H), 6.62 (dd, J=8.9, 4.2 Hz, 1H), 5.13 (d, J=5.5 Hz, 1H), 4.26-4.36 (m, 1H), 3.84-3.90 (m, 1H), 3.79 (d, J=1.6 Hz, 6H), 3.38 (s, 2H), 3.22-3.32 (m, 1H), 3.13 (dd, J=9.8, 3.2 Hz, 1H), 2.64 (ddd, J=12.9, 8.3, 4.3 Hz, 1H), 2.05 ppm (s, 4H); LCMS (M−H⁺): 567, purity: 94%.

### Synthesis of 3-((2R, 3R, 4R, 5R)-5-((bis(4-methoxyphenyl)(phenyl)methoxy)methyl)-4-hydroxy-3-methoxytetrahydrofuran-2-yl)pyrimidine-2, 4(1H, 3H)-dione (WV-NU-254)

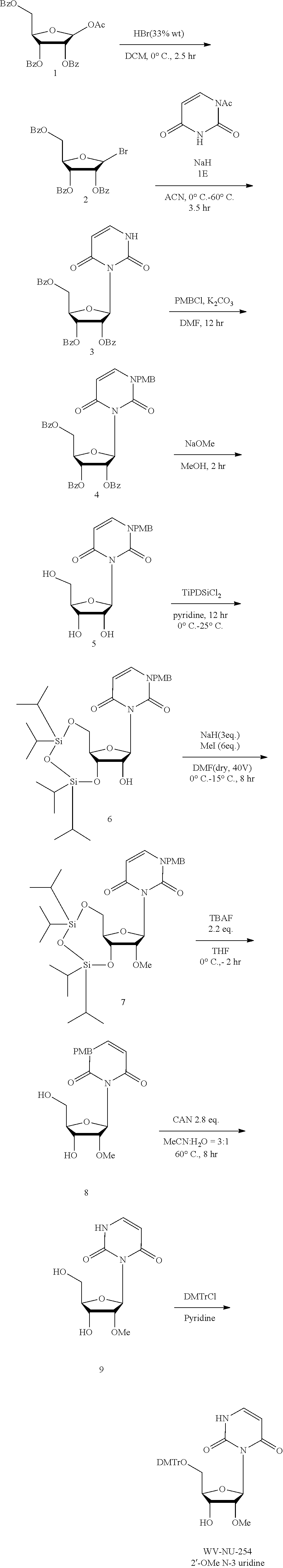

Step 1. To a solution of compound 1 (200 g, 396.44 mmol) in DCM (2000 mL) was added hydrogen bromide (388.81 g, 260.95 mL, 33% purity) at 0° C. in N₂. The mixture was stirred at 0° C. for 2 hr. TLC indicated compound 1 was consumed completely and two new spots formed. The mixture of reaction was poured into 500 mL of ice water, and the organic phase was separated, washed with water (300 mL*5), dried over Na₂SO₄, and evaporated under reduced pressure. Then the remaining acetic acid was removed and the crude product was dried in vacuous. Compound 2 (208 g, crude) was obtained as a yellow oil.

Step 2. For four batches: To a solution of compound 1E (22.88 g, 148.47 mmol) in ACN (2000 mL) was added NaH (7.92 g, 60% purity) at 0° C. in N₂ for 30 min. And then added compound 2 (52 g, 98.98 mmol) for 30 min at 25° C., latter 1 hr heat up to 40° C. And then the mixture was stirred at 60° C. for 2 hr. TLC indicated compound 2 was consumed completely and four new spots formed. Filtration to remove solids, the organic phase vacuum concentration to obtain coarse product. The residue was purified by column chromatography (SiO₂, Petroleum ether:Ethyl acetate=1:0 to 0:1). Compound 3 (60 g, 27.27% yield) was obtained as a yellow solid. ¹HNMR (400 MHz, DMSO-d6) 6=11.40 (br s, 1H), 7.98 (br d, J=7.6 Hz, 2H), 7.91 (br d, J=7.6 Hz, 2H), 7.83 (br d, J=7.6 Hz, 2H), 7.69-7.57 (m, 3H), 7.54 (br d, J=7.5 Hz, 1H), 7.50-7.42 (m, 4H), 7.42-7.34 (m, 2H), 6.51 (s, 1H), 6.12-6.05 (m, 2H), 5.68 (d, J=7.6 Hz, 1H), 4.72-4.62 (m, 2H), 4.57-4.48 (m, 1H); LCMS (M+H⁺): 579.3, purity: 94.16%.

Step 3. To a solution of compound 3 (20 g, 35.94 mmol) in DMF (300 mL) was added K₂CO₃ (7.45 g, 53.91 mmol) and PMB-C1 (6.19 g, 39.53 mmol). The mixture was stirred at 25° C. for 12 hr. TLC indicated compound 3 was consumed completely and one new spot formed. The reaction mixture was diluted with water 200 mL and extracted with EtOAc (100 mL*3). The combined organic layers were dried over Na₂SO₄ filtered and concentrated under reduced pressure to give a residue. The residue was purified by column chromatography (SiO₂, Petroleum ether:Ethyl acetate=15:1 to 0:1). Compound 4 (23 g, 94.58% yield) was obtained as a yellow oil. LCMS (M+H⁺): 700.3, purity: 98.35%.

Step 4. To a solution of compound 4 (23 g, 33.99 mmol) was in MeOH (300 mL) and then added NaOMe (5.51 g, 101.97 mmol). The mixture was stirred at 25° C. for 3 hr. TLC indicated compound 4 was consumed completely and two new spot formed. The reaction was added NH₄C1 (5.6 g) and stirred at room temperature for 10 min, and then vacuum concentration. The residue was purified by column chromatography (SiO₂, Petroleum ether:Ethyl acetate=5:1 to 0:1 to Ethyl acetate:MeOH=3:1). Compound 5 (12 g, 96.90% yield) was obtained as a yellow oil. ¹H NMR (400 MHz, DMSO-d6) δ=7.84 (d, J=7.9 Hz, 1H), 7.28 (d, J=8.6 Hz, 2H), 6.98-6.87 (m, 2H), 6.07 (d, J=3.4 Hz, 1H), 5.70 (d, J=7.9 Hz, 1H), 5.05 (br s, 1H), 4.87-4.77 (m, 2H), 4.58 (br s, 1H), 4.43 (br dd, J=3.5, 5.8 Hz, 1H), 4.09 (br t, J=6.3 Hz, 1H), 3.73 (s, 3H), 3.70-3.52 (m, 2H), 3.45-3.29 (m, 2H); LCMS (M+H+): 365.2, purity: 99.51%.

Step 5. To a solution of 3-((2R, 3R, 4S, 5R)-3, 4-dihydroxy-5-(hydroxymethyl)tetrahydrofuran-2-yl)-1-(4-methoxybenzyl)pyrimidine-2, 4(1H, 3H)-dione (5) (12 g, 32.94 mmol) in Pyridine (200 mL) was added chloro-[chloro(diisopropyl)silyl]oxy-diisopropyl-silane (13.51 g, 42.82 mmol) slowly at 0° C. for 1 hr in N₂. The mixture was stirred at 0° C.-25° C. for 1 1 hr. LCMS showed compound 5 was consumed completely and desired mass was detected. The reaction mixture was filtered and concentrated under reduced pressure to give a residue. The residue was purified by column chromatography (SiO₂, Petroleum ether:Ethyl acetate=20:1 to 0:1). 3-((6aR,8R,9R,9aS)-9-hydroxy-2, 2, 4, 4-tetraisopropyltetrahydro-6H-furo[3, 2-f][1, 3, 5, 2, 4]trioxadisilocin-8-yl)-1-(4-methoxybenzyl)pyrimidine-2, 4(1H, 3H)-dione (6) (15 g, 75.05% yield) was obtained as a yellow oil. LCMS (M+H+): 607.4, purity: 95.88%.

Step 6. For five batches: To a solution of 3-((6aR, 8R, 9R, 9aS)-9-hydroxy-2, 2, 4, 4-tetraisopropyltetrahydro-6H-furo[3, 2-f][1, 3, 5, 2, 4]trioxadisilocin-8-yl)-1-(4-methoxybenzyl)pyrimidine-2, 4(1H, 3H)-dione (6) (5 g, 8.24 mmol) in DMF (200 mL) was added NaH (988.61 mg, 24.72 mmol, 60% purity) and Mel (7.02 g, 49.44 mmol) at 0° C. in N₂. The mixture was stirred at 0° C.-15° C. for 10 hr. LCMS showed compound 6 was consumed completely and desired mass was detected. The reaction of five batches mixture were quenched by addition NH₄Cl 1000 mL, and then diluted with H₂O 500 mL and extracted with EtOAc (500 mL*4). The combined organic layers were dried over Na₂SO₄ filtered and concentrated under reduced pressure to give a residue. The residue was purified by column chromatography (SiO₂, Petroleum ether:Ethyl acetate=10:1 to 0:1). 1-(4-methoxybenzyl)-3-((6aR, 8R, 9R, 9aR)-2, 2, 4, 4-tetraisopropyl-9-methoxytetrahydro-6H-furo[3, 2-f][1, 3, 5, 2, 4]trioxadisilocin-8-yl)pyrimidine-2, 4(1H, 3H)-dione (7) (21.5 g, 84.06% yield) was obtained as a yellow oil. ¹H NMR (400 MHz, CHLOROFORM-d) δ=7.23 (d, J=8.6 Hz, 2H), 7.07 (d, J=8.0 Hz, 1H), 6.91-6.87 (m, 2H), 6.26 (d, J=1.6 Hz, 1H), 5.64 (d, J=8.0 Hz, 1H), 5.06 (dd, J=5.9, 9.1 Hz, 1H), 4.90 (d, J=14.6 Hz, 1H), 4.69 (d, J=14.5 Hz, 1H), 4.22 (dd, J=1.5, 5.9 Hz, 1H), 4.07 (d, J=2.8 Hz, 1H), 4.01-3.96 (m, 1H), 3.86-3.80 (m, 4H), 3.51 (s, 3H), 1.17-1.01 (m, 28H); LCMS (M+H+): 621.4, purity: 99.09%.

Step 7. To a solution of 1-(4-methoxybenzyl)-3-((6aR, 8R, 9R, 9aR)-2, 2, 4, 4-tetraisopropyl-9-methoxytetrahydro-6H-furo[3, 2-f][1, 3, 5, 2, 4]trioxadisilocin-8-yl)pyrimidine-2, 4(1H, 3H)-dione (7) (19 g, 30.60 mmol) in THF (200 mL) was added TBAF (1 M, 61.20 mL). The mixture was stirred at 0° C. for 4 hr. TLC indicated compound 7 was consumed completely and one new spot formed. The reaction mixture was filtered and concentrated under reduced pressure to give a residue. The residue was purified by column chromatography (SiO₂, Petroleum ether:Ethyl acetate=1:1 to 0:1 to Ethyl acetate:MeOH=1:1). 3-((2R, 3R, 4R, 5R)-4-hydroxy-5-(hydroxymethyl)-3-methoxytetrahydrofuran-2-yl)-1-(4-methoxybenzyl)pyrimidine-2, 4(1H, 3H)-dione (8) (10.9 g, 94.14% yield) was obtained as a white solid. ¹H NMR (400 MHz, DMSO-d6) 6=7.84 (d, J=7.9 Hz, 1H), 7.29 (d, J=8.6 Hz, 2H), 6.93 (d, J=8.6 Hz, 2H), 6.12 (d, J=3.3 Hz, 1H), 5.71 (d, J=7.9 Hz, 1H), 4.88 (d, J=7.1 Hz, 1H), 4.83 (d, J=7.1 Hz, 2H), 4.58 (t, J=5.8 Hz, 1H), 4.27-4.14 (m, 2H), 3.74 (s, 3H), 3.66 (dt, J=3.3, 6.6 Hz, 1H), 3.60 (ddd, J=3.4, 5.3, 11.7 Hz, 1H), 3.39 (td, J=6.3, 12.0 Hz, 1H), 3.30 (s, 3H); LCMS (M+H⁺): 401.2, purity: 96.37%.

Step 8. To a solution of 3-((2R, 3R, 4R, 5R)-4-hydroxy-5-(hydroxymethyl)-3-methoxytetrahydrofuran-2-yl)-1-(4-methoxybenzyl)pyrimidine-2, 4(1H, 3H)-dione (8) (10.9 g, 28.81 mmol) in ACN (400 mL) and H₂O (100 mL) was added CAN (39.48 g, 72.02 mmol). The mixture was stirred at 60° C. for 3 hr. TLC indicated compound 8 was consumed completely and two new spot formed. The reaction mixture was filtered and concentrated under reduced pressure to give a residue. The crude product was purification by prep-HPLC (column: Welch Xtimate C18 250*70 mm #10 um; mobile phase: [water (NH₄HCO₃)-ACN]; B %: 0%-10%, 20 min). 3-((2R, 3R, 4R, 5R)-4-hydroxy-5-(hydroxymethyl)-3-methoxytetrahydrofuran-2-yl)pyrimidine-2, 4(1H, 3H)-dione (9) (3.6 g, 48.65% yield) was obtained as a white solid. ¹H NMR (400 MHz, DMSO-d6) δ=11.17 (br s, 1H), 7.46 (d, J=7.6 Hz, 1H), 6.10 (d, J=3.1 Hz, 1H), 5.58 (d, J=7.6 Hz, 1H), 4.86 (d, J=6.9 Hz, 1H), 4.58 (t, J=5.8 Hz, 1H), 4.24-4.16 (m, 2H), 3.66 (dt, J=3.3, 6.3 Hz, 1H), 3.60 (ddd, J=3.4, 5.3, 11.8 Hz, 1H), 3.44-3.36 (m, 1H), 3.31 (s, 3H), 3.17 (d, J=5.1 Hz, 1H); LCMS (M+H⁺):281.1, purity: 100.0%.

To a solution of 3-((2R, 3R, 4R, 5R)-4-hydroxy-5-(hydroxymethyl)-3-methoxytetrahydrofuran-2-yl)pyrimidine-2, 4(1H, 3H)-dione (9) (3.5 g, 13.55 mmol) in Pyridine (50 mL) was added DMT-Cl (4.59 g, 13.55 mmol). The mixture was stirred at 15° C. for 6 hr. TLC indicated compound 9 was consumed completely and two new spot formed. The reaction mixture was filtered and concentrated under reduced pressure to give a residue. The residue was purified by column chromatography (SiO₂, Petroleum ether:Ethyl acetate=10:1 to 0:1 to Ethyl acetate:MeOH=5:1, 5% TEA). 3-((2R, 3R, 4R, 5R)-5-((bis(4-methoxyphenyl)(phenyl)methoxy)methyl)-4-hydroxy-3-methoxytetrahydrofuran-2-yl)pyrimidine-2. 4(1H, 3H)-dione (WV-NU-254) (6.3 g, 82.89% yield) was obtained as a white solid. ¹H NMR (400 MHz, CHLOROFORM-d) δ=9.36 (br s, 1H), 7.47 (d, J=7.8 Hz, 2H), 7.35 (d, J=8.6 Hz, 4H), 7.26-7.22 (m, 2H), 7.20-7.15 (m, 1H), 6.85-6.74 (m, 5H), 6.36 (d, J=1.8 Hz, 1H), 5.62 (d, J=7.8 Hz, 1H), 4.54-4.45 (m, 1H), 4.21 (dd, J=1.9, 6.6 Hz, 1H), 3.93 (dt, J=3.1, 7.1 Hz, 1H), 3.77 (s, 6H), 3.47 (s, 3H), 3.43 (dd, J=3.0, 10.1 Hz, 1H), 3.31 (dd, J=6.6, 10.1 Hz, 1H), 2.70 (d, J=9.1 Hz, 1H); LCMS (M−H⁺):559.3, purity: 99.10%.

### Synthesis of 3-((2R, 4S, 5R)-5-((bis(4-methoxyphenyl)(phenyl)methoxy)methyl)-4-hydroxytetrahydrofuran-2-yl)-5-ethylpyrimidine-2, 4(1H, 3H)-dione (WV-NU-256)

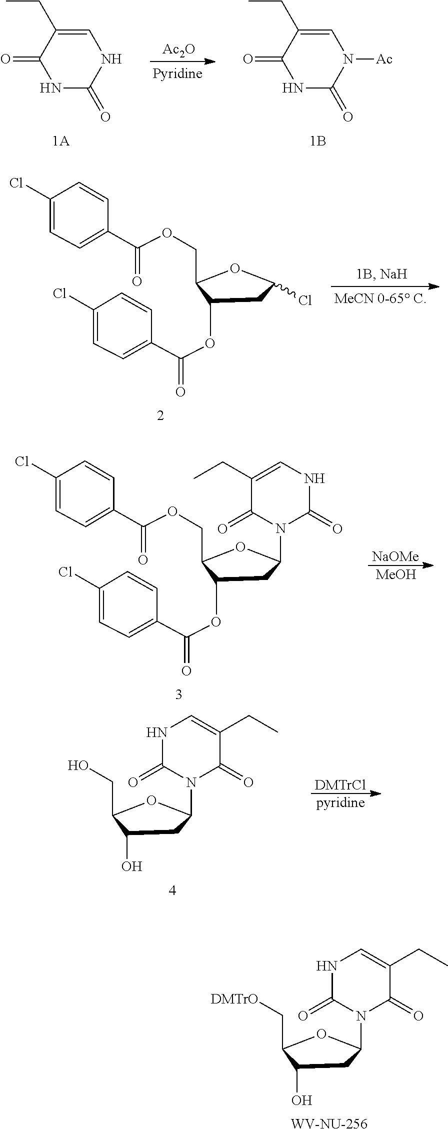

Step 1. To a solution of 5-ethylpyrimidine-2, 4(1H, 3H)-dione (1A) (50 g, 356.79 mmol) in Pyridine (500 mL) was added Ac₂O (218.54 g, 2.14 mol). The mixture was stirred at 120° C. for 3 hr. TLC indicated compound 1A was consumed completely and one new spot formed. The reaction mixture was concentrated under reduced pressure to give a crude product. The crude product was washed with Ethyl acetate (500 mL*3), filtered and the cake was dried under reduced pressure to get the product. 1-Acetyl-5-ethylpyrimidine-2, 4(1H, 3H)-dione (1B) (50 g, 76.92% yield) was obtained as a white solid. ¹HNMR (400 MHz, DMSO-d6) δ=11.55 (br s, 1H), 7.93 (s, 1H), 2.60 (s, 3H), 2.28 (q, J=7.4 Hz, 2H), 1.04 (t, J=7.4 Hz, 3H).

Step 2. Four batches: A clean and dry three-neck 2L round bottom flask charge with 1-acetyl-5-ethylpyrimidine-2, 4(1H, 3H)-dione (1B) (10 g, 54.89 mmol) and dissolved into dry ACN (1000 mL) under argon atmosphere. Cool the reaction mixture to 0° C. by using ice bath. Then NaH (3.29 g, 82.34 mmol, 60% purity) was added portion wise to the reaction mixture and stir for 30 min at 0° C. After that, compound 2 (32.78 g, 76.30 mmol) was added portion wise and stirred the reaction mixture for 30 min at 0° C. and 65° C. for 3 hr. LCMS showed compound 2 was consumed completely and one main peak with desired mass was detected. Cool the reaction mixture to rt. and filtered, the filtrate was concentrated under reduced pressure to give a crude product. The residue was purified by column chromatography (SiO₂, Petroleum ether/Ethyl acetate=1/0 to 0/1). The crude product was purified by re-crystallization from Petroleum ether:Ethyl acetate =5:1 (200 mL) at 60° C. ((2R, 3S, 5R)-3-((4—Chlorobenzoyl)oxy)-5-(5-ethyl-2, 6-dioxo-3, 6-dihydropyrimidin-1(2H)-yl)tetrahydrofuran-2-yl)methyl 4-chlorobenzoate (3) (5.7 g, 19.00% yield) was obtained as a white solid and 10 g crude.

Step 3. To a solution of ((2R, 3S, 5R)-3-((4-chlorobenzoyl)oxy)-5-(5-ethyl-2, 6-dioxo-3, 6-dihydropyrimidin-1(2H)-yl)tetrahydrofuran-2-yl)methyl 4-chlorobenzoate (3) (7.5 g, 14.06 mmol) in MeOH (100 mL) was added NaOMe (1.90 g, 35.15 mmol). The mixture was stirred at 20° C. for 12 hr. LCMS showed compound 3 was consumed completely and one main peak with desired mass was detected. The reaction mixture was added dry NH₄C1 (1.9 g) and stirred. The reaction mixture was concentrated under reduced pressure to remove MeOH. The residue was purified by column chromatography (SiO₂, Petroleum ether/Ethyl acetate=1/0 to 0/1). 5-Ethyl-3-((2R, 4S, 5R)-4-hydroxy-5-(hydroxymethyl)tetrahydrofuran-2-yl)pyrimidine-2, 4(1H, 3H)-dione (4) (3.4 g, 87.83% yield) was obtained as a white solid. ¹H NMR (400 MHz, DMSO-d₆) δ=10.89 (br d, J=4.9 Hz, 1H), 7.22 (d, J=5.1 Hz, 1H), 6.57 (dd, J=6.6, 7.8 Hz, 1H), 5.05 (d, J=4.8 Hz, 1H), 4.54 (dd, J=5.1, 6.4 Hz, 1H), 4.35-4.26 (m, 1H), 3.69-3.63 (m, 1H), 3.62-3.54 (m, 1H), 3.48-3.40 (m, 1H), 3.17 (d, J=5.3 Hz, 1H), 2.74-2.66 (m, 1H), 2.19 (q, J=7.4 Hz, 2H), 1.00 (t, J=7.4 Hz, 3H); LCMS (M−H⁺):255.2.

Step 4. To a solution of 5-ethyl-3-((2R, 4S, 5R)-4-hydroxy-5-(hydroxymethyl)tetrahydrofuran-2-yl)pyrimidine-2, 4(1H, 3H)-dione (4) (3.4 g, 13.27 mmol) in Pyridine (90 mL) was added DMTCl (4.95 g, 14.59 mmol). The mixture was stirred at 20° C. for 12 hr. LCMS showed compound 4 was consumed completely and one main peak with desired mass was detected. The reaction was diluted with H₂O 100 mL and extracted with Ethyl acetate (100 mL*3). The combined organic layers were washed with saturated salt water (100 mL*2), dried over with dry Na₂SO₄, filtered and concentrated under reduced pressure to give a residue. The residue was purified by column chromatography (SiO₂, Petroleum ether/Ethyl acetate=1/0 to 0/1). 3-((2R, 4S, 5R)-5-((Bis(4-methoxyphenyl)(phenyl)methoxy)methyl)-4-hydroxytetrahydrofuran-2-yl)-5-ethylpyrimidine-2, 4(1H, 3H)-dione (WV-NU-256) (5.8 g, 76.24% yield) was obtained as a white solid. ¹HNMR (400 MHz, DMSO-d₆) δ=10.85 (br s, 1H), 7.42-7.36 (m, 2H), 7.29-7.22 (m, 7H), 7.21-7.16 (m, 1H), 6.87-6.79 (m, 4H), 6.62 (dd, J=4.1, 9.1 Hz, 1H), 5.05 (d, J=5.5 Hz, 1H), 4.31-4.24 (m, 1H), 3.86-3.80 (m, 1H), 3.72 (d, J=2.9 Hz, 6H), 3.25-3.18 (m, 1H), 3.06 (dd, J=3.2, 9.7 Hz, 1H), 2.59 (ddd, J=4.2, 8.4, 12.9 Hz, 1H), 2.07-2.00 (m, 1H), 1.17 (t, J=7.2 Hz, 2H), 1.01-0.95 (m, 3H); LCMS (M−H⁺):557.3, LCMS purity: 97.4%.

### Synthesis of 3-((2R, 4S, 5R)-5-((bis(4-methoxyphenyl)(phenyl)methoxy)methyl)-4-hydroxytetrahydrofuran-2-yl)-5-fluoropyrimidine-2, 4(1H, 3H)-dione (WV-NU-271)

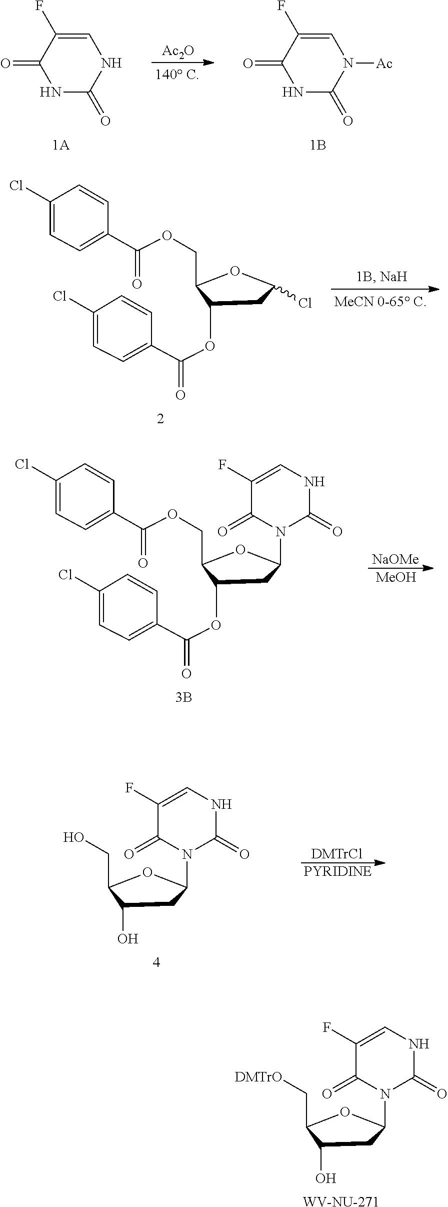

Step 1. To a solution of 5-fluoropyrimidine-2, 4(1H, 3H)-dione (1A) (52 g, 399.76 mmol) was added Ac₂O (545.00 g, 5.34 mol). The mixture was stirred at 140° C. for 12 hr. TLC indicated compound 1A was consumed completely and one new spot formed. The reaction mixture was concentrated under reduced pressure to give a crude product. The crude product was washed with Petroleum ether:Ethyl acetate=10:1(100 mL*3), filtered and the cake was dried under reduced pressure to get the product. 1-Acetyl-5-fluoropyrimidine-2, 4(1H, 3H)-dione (1B) (48 g, 69.77% yield) was obtained as a white solid. ¹HNMR (400 MHz, DMSO-d6) δ=12.15-12.02 (m, 1H), 8.33 (d, J=7.5 Hz, 1H), 2.60 (s, 3H).

Step 2. A clean and dry three-neck 3 L round bottom flask charge with 1-acetyl-5-fluoropyrimidine-2, 4(1H, 3H)-dione (1B) (18 g, 104.58 mmol) and dissolved into dry ACN (1800 mL) under argon atmosphere. Cool the reaction mixture to 0° C. by using ice bath. Then NaH (6.27 g, 156.87 mmol, 60% purity) was added portion wise to the reaction mixture and stir for 30 min at 0° C. After that, compound 2 (62.46 g, 145.37 mmol) was added portion wise and stir the reaction mixture for 30 min at 0° C. and 65° C. for 3 hr. LCMS showed compound 2 was consumed completely and one main peak with desired mass was detected. Cool the reaction mixture to rt. and filtered, the filtrate was concentrated under reduced pressure to give a crude product. The crude product was triturated with Ethyl acetate (200 mL) and Petroleum ether (600 mL) at 65° C. for 15 min, filtered and the cake washed for three times, the cake was dried under vacuum to get the product and all the mother liquor was concentrated to get the crude. ((2R, 3S, 5R)-3-((4-Chlorobenzoyl)oxy)-5-(5-fluoro-2, 6-dioxo-3, 6-dihydropyrimidin-1(2H)-yl)tetrahydrofuran-2-yl)methyl 4-chlorobenzoate (3B) (14 g, 25.58% yield) was obtained as a white solid.

Step 3. To a solution of ((2R, 3S, 5R)-3-((4-chlorobenzoyl)oxy)-5-(5-fluoro-2, 6-dioxo-3, 6-dihydropyrimidin-1(2H)-yl)tetrahydrofuran-2-yl)methyl 4-chlorobenzoate (3B) (14 g, 26.75 mmol) in MeOH (200 mL) was added NaOMe (3.61 g, 66.88 mmol). The mixture was stirred at 20° C. for 2 hr. LCMS showed compound 3B was consumed completely and one main peak with desired mass was detected. The reaction mixture was quenched with NH₄C1 solid (3.6 g), filtrate the solid and get filtrate. The residue was purified by column chromatography (SiO₂, Petroleum ether/Ethyl acetate=1/0 to 0/1, Dichloromethane: Methanol=1/0 to 0/1). 5-fluoro-3-((2R, 4S, 5R)-4-hydroxy-5-(hydroxymethyl)tetrahydrofuran-2-yl)pyrimidine-2, 4(1H, 3H)-dione (4) (5.74 g, 87.10% yield) was obtained as a white foam. ¹H NMR (400 MHz, DMSO-d₆) δ=11.47-10.66 (m, 1H), 7.83 (d, J=5.4 Hz, 1H), 6.49 (dd, J=6.1, 7.9 Hz, 1H), 5.20-4.98 (m, 1H), 4.54 (br s, 1H), 4.29 (td, J=4.8. 7.3 Hz, 1H), 3.69-3.52 (m, 2H), 3.48-3.39 (m, 1H), 2.71 (ddd, J=6.2, 7.2, 13.1 Hz, 1H), 2.01-1.89 (m, 1H); LCMS (M+H⁺): 269.2.

Step 4. To a solution of 5-fluoro-3-((2R, 4S, 5R)-4-hydroxy-5-(hydroxymethyl)tetrahydrofuran-2-yl)pyrimidine-2, 4(1H, 3H)-dione (4) (5.7 g, 23.15 mmol) in pyridine (140 mL) was added DMTCl (8.63 g, 25.47 mmol). The mixture was stirred at 20° C. for 5 hr. LCMS showed compound 4 was consumed completely and one main peak with desired mass was detected. The reaction mixture was concentrated under reduced pressure to remove pyridine. The residue was diluted with H20 150 mL and extracted with Ethyl acetate (150 mL *3). The combined organic layers were washed with salt water 150 mL, dried over Na₂SO₄, filtered and concentrated under reduced pressure to give a residue. The residue was purified by column chromatography (SiO₂, Petroleum ether/Ethyl acetate=1/0 to 0/1) to get 3-((2R, 4S, 5R)-5-((bis(4-methoxyphenyl)(phenyl)methoxy)methyl)-4-hydroxytetrahydrofuran-2-yl)-5-fluoropyrimidine-2, 4(1H, 3H)-dione (WV-NU-271) (10.4 g, 79.56%) as a white solid. ¹H NMR (400 MHz, DMSO-d₆) δ=11.04 (br s, 1H), 7.87 (d, J=5.4 Hz, 1H), 7.39 (br d, J=7.5 Hz, 2H), 7.30-7.22 (m, 6H), 6.84 (br t, J=9.0 Hz, 4H), 6.59-6.52 (m, 1H), 5.10 (br d, J=5.1 Hz, 1H), 4.31-4.21 (m, 1H), 3.88-3.80 (m, 1H), 3.73 (d, J=2.1 Hz, 7H), 3.27-3.18 (m, 1H), 3.08 (br dd, J=3.2, 9.7 Hz, 1H), 2.62 (ddd, J=4.2, 8.3, 12.9 Hz, 1H), 2.09-2.01 (m, 1H); LCMS (M−H⁺): 547.2, LCMS purity: 97.15%.

### Synthesis of 3-((2R, 4S, 5R)-5-((bis(4-methoxyphenyl)(phenyl)methoxy)methyl)-4-hydroxytetrahydrofuran-2-yl)-[1, 2, 4]triazolo[4, 3-a]pyrazin-8(7H)-one (WV-NU-266)

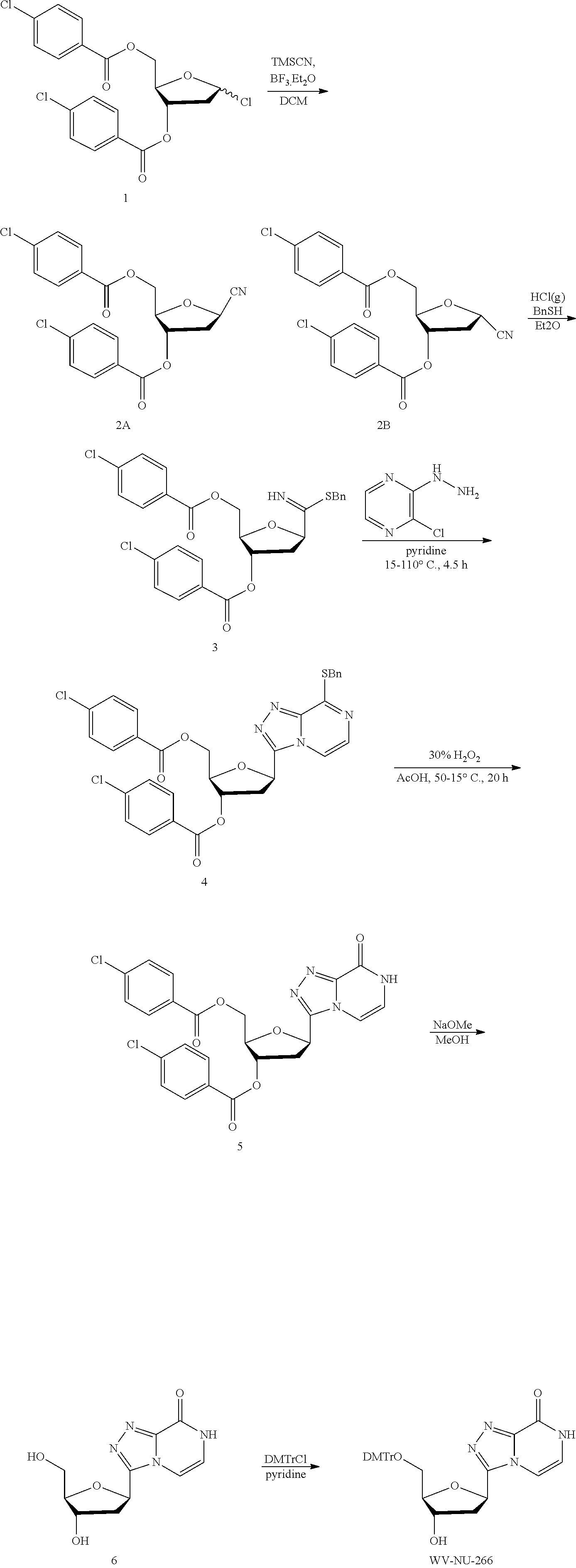

Step 1. To a solution of compound 1 (70.00 g, 162.91 mmol) in DCM (1400 mL) was added TMSCN (24.24 g, 244.37 mmol.) and BF₃·Et₂O (46.24 g, 325.83 mmol.). The mixture was stirred at 15° C. for 12 hr. TLC indicated compound 1 was consumed completely and two new spots formed. The reaction was quenched with saturated ammonium bicarbonate solution (200 mL) and stirred for additional 30 min. The slurry of two batches were combined and extracted with CH₂Cl₂ (2*1000 mL). The combined organic phases were dried over Na₂SO₄ and evaporated to dryness. The crude product was triturated with 1000 mL mixed solvent of (Petroleum ether:Ethyl acetate=1:1) at 15° C. for 30 min for two times. ((2R, 3S, 5R)-3-((4-Chlorobenzoyl)oxy)-5-cyanotetrahydrofuran-2-yl)methyl 4-chlorobenzoate (2A) (62 g, 45.29% yield) was obtained as a gray solid. ¹H NMR (400 MHz, DMSO-d₆) 6=8.05-7.96 (m, 4H), 7.62 (d, J=8.1 Hz, 4H), 5.68-5.56 (m, 1H), 5.18 (t, J=7.9 Hz, 1H), 4.59-4.54 (m, 1H), 4.52-4.43 (m, 2H), 2.72 (dd, J=4.1, 8.0 Hz, 2H).

Step 2. A solution of ((2R, 3S, 5R)-3-((4-chlorobenzoyl)oxy)-5-cyanotetrahydrofuran-2-yl)methyl 4-chlorobenzoate (2A) (52 g, 123.74 mmol) and BnSH (31.34 g, 252.33 mmol) in Et₂O (1500 mL) was saturated at 0° C. with anhydrous HCl for 2 h (after 45 min, a white precipitate formed). The mixture was then kept at −10° C. for 6 h. LCMS showed the desired mass was detected. The white precipitate was isolated by filtration and washed with anhydrous Et₂O 500 mL, and the cake was dried to get the product. (2R, 3S, 5R)-5-((Benzylthio)(imino)methyl)-2-(((4-chlorobenzoyl)oxy)methyl)tetrahydrofuran-3-yl 4-chlorobenzoate (3) (67 g, crude) was obtained as a white solid. LCMS (M+H⁺):545.1.

Step 3. The mixture of (2R, 3S, 5R)-5-((benzylthio)(imino)methyl)-2-(((4-chlorobenzoyl)oxy)methyl)tetrahydrofuran-3-yl 4-chlorobenzoate (3) (37 g, 67.96 mmol) and (3-chloropyrazin-2-yl)hydrazine (19.65 g, 135.92 mmol) in Py (1500 mL) were stirred for 3 h at 15° C. and then 115° C. for 1.5 h. LCMS showed the desired mass was detected. The reaction mixture was concentrated under reduced pressure to remove solvent. The residue was purified by column chromatography (SiO₂, Petroleum ether/Ethyl acetate=1/0 to 0/1). (2R, 3S, 5R)-5-(8-(Benzylthio)-[1, 2, 4]triazolo[4, 3-a]pyrazin-3-yl)-2-(((4-chlorobenzoyl)oxy)methyl)tetrahydrofuran-3-yl 4-chlorobenzoate (4) (14 g, 32.42% yield) was obtained as a yellow solid. LCMS (M+H+):635.2.

Step 4. To a solution of (2R, 3S, 5R)-5-(8-(benzylthio)-[1, 2, 4]triazolo[4, 3-a]pyrazin-3-yl)-2-(((4-chlorobenzoyl)oxy)methyl)tetrahydrofuran-3-yl 4-chlorobenzoate (4) (22 g, 34.62 mmol) in AcOH (220 mL) was added H₂O₂(21.81 g, 192.36 mmol, 30% purity) at 50° C. The mixture was stirred at 15° C. for 20 hr. LCMS showed the desired mass was detected. The AcOH was removed in vacuo to a light yellow syrup to which H₂O (500 mL) was added. The pH of this solution was brought to 6 with 3 N NaOH solution. The resulting precipitate was isolated by filtration and recrystallized from 200 mL MeOH to give a crude. ((2R, 3S, 5R)-3-((4—Chlorobenzoyl)oxy)-5-(8-oxo-7, 8-dihydro-[1, 2, 4]triazolo[4, 3-a]pyrazin-3-yl)tetrahydrofuran-2-yl)methyl 4-chlorobenzoate (5) (18.32 g, crude) was obtained as a yellow solid. LCMS (M+H⁺): 529.1.

Step 5. To a solution of ((2R, 3S, 5R)-3-((4-chlorobenzoyl)oxy)-5-(8-oxo-7, 8-dihydro-[1, 2, 4]triazolo[4, 3-a]pyrazin-3-yl)tetrahydrofuran-2-yl)methyl 4-chlorobenzoate (5) (18.32 g, 34.61 mmol.) in MeOH (200 mL) was added NaOMe (4.67 g, 86.52 mmol). The mixture was stirred at 15° C. for 2 hr. LCMS showed the desired mass was detected. The reaction mixture was quenched by addition NH₄C1 4.7 g, and then concentrated under reduced pressure to give a residue. 3-((2R, 4S, 5R)-4—Hydroxy-5-(hydroxymethyl)tetrahydrofuran-2-yl)-[l, 2, 4]triazolo[4, 3-a]pyrazin-8(7H)-one (6) (8.73 g, crude) was obtained as a white solid. LCMS (M+H+):253.1.

Step 6. To a solution of 3-((2R, 4S, 5R)-4-hydroxy-5-(hydroxymethyl)tetrahydrofuran-2-yl)-[1, 2, 4]triazolo[4, 3-a]pyrazin-8(7H)-one (6) (8.58 g, 34.02 mmol.) in Py (200 mL) was added DMTCl (11.53 g, 34.02 mmol). The mixture was stirred at 15° C. for 10 hr. LCMS showed the desired mass was detected. The reaction mixture was quenched and diluted by addition sat. NaHCO₃ 100 mL, and then extracted with ethyl acetate 200 mL (100 mL*2). The combined organic layers were washed with sat. brine 200 mL, dried over Na₂SO₄, filtered and concentrated under reduced pressure to give a residue. The crude product was triturated with DCM 100 mL*3 for 15 min. 3-((2R, 4S, 5R)-5-((Bis(4-methoxyphenyl)(phenyl)methoxy)methyl)-4-hydroxytetrahydrofuran-2-yl)-[1, 2, 4]triazolo[4, 3-a]pyrazin-8(7H)-one (WV-NU-266) (6.08 g, 31.07% yield, ) was obtained as a white solid. ¹H NMR (400 MHz, DMSO-d₆) δ=11.49 (br d, J=5.4 Hz, 1H), 7.54 (d, J=5.6 Hz, 1H), 7.26-7.13 (m, 5H), 7.10 (d, J=8.8 Hz, 4H), 6.83-6.74 (m, 5H), 5.58 (t, J=7.1 Hz, 1H), 5.35 (br s, 1H), 4.38 (br s, 1H), 3.98 (q, J=3.9 Hz, 1H), 3.71 (s, 6H), 3.08-2.87 (m, 2H), 2.83 (td, J=6.6, 13.2 Hz, 1H), 2.30 (ddd, J=4.0, 6.9, 12.6 Hz, 1H); LCMS (M−H⁺): 553.2, purity: 96.42%.

### Synthesis of (2R, 3S, 4R, 5R)-5-((bis(4-methoxyphenyl)(phenyl)methoxy)methyl)-2-(2, 4-dioxo-3, 4-dihydropyrimidin-1(2H)-yl)-4-(((1S, 3S, 3aS)-3-((phenylsulfonyl)methyl)tetrahydro-1H, 3H-pyrrolo[1, 2-c][1,3,2]oxazaphoshol-1-yl)oxy)tetrahydrofuran-3-yl acetate

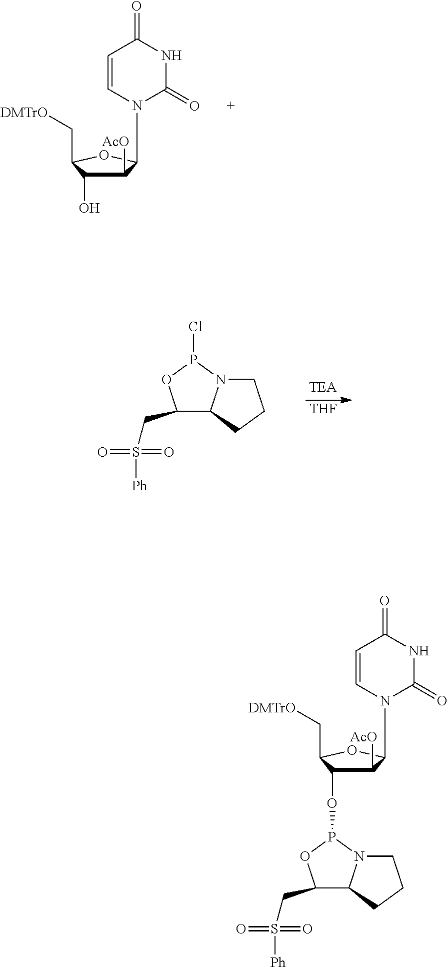

To a solution of dry [(2R, 3R, 5R)-5-[[bis(4-methoxyphenyl)-phenyl-methoxy]methyl]-2-(2, 4-dioxopyrimidin-1-yl)-4-hydroxy-tetrahydrofuran-3-yl]acetate (9.5 g, 16.14 mmol) in THF (70 mL) was added triethylamine (4.95 mL, 35.51 mmol). The reaction flask was set in a water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.43Min THF, 56.3 mL, 24.21 mmol) was added fast dropwise. The water bath was removed. The cloudy reaction solution was stirred at rt for 1 hr. TLC showed the reaction was complete. The reaction was quenched by water (145 μL). Anhydrous MgSO₄ (1.937 g) was added. The mixture was filtered through celite, and the filtrate was concentrated to afford the crude product as an off-white foam. The crude product was purified by normal phase column chromatography applying 20-100% EtOAc in hexanes (each mobile phase contained 1% triethylamine) as the gradient to afford the title compound as a white foam (10.96 g, 77.9% yield). ¹H NMR (600 MHz, Chloroform-d) δ 8.52 (s, 1H), 7.92-7.87 (m, 2H), 7.65-7.59 (m, 1H), 7.53 (q, J=7.9 Hz, 3H), 7.46-7.41 (m, 2H), 7.34-7.28 (m, 6H), 7.24 (t, J=7.3 Hz, 1H), 6.87-6.82 (m, 4H), 6.26 (d, J=4.6 Hz, 1H), 5.48 (d, J=8.2 Hz, 1H), 5.36 (dd, J=4.6, 2.9 Hz, 1H), 5.04 (q, J=6.1 Hz, 1H), 4.68 (ddd, J=9.1, 4.5, 2.9 Hz, 1H), 4.02 (q, J=4.4 Hz, 1H), 3.79 (s, 6H), 3.64 (dq, J=10.0, 6.1 Hz, 1H), 3.49 (ddd, J=14.1, 10.5, 7.3 Hz, 1H), 3.44 (dt, J=14.4, 5.3 Hz, 2H), 3.38-3.31 (m, 2H), 3.09-3.00 (m, 1H), 1.95 (s, 3H), 1.86 (ddt, J=12.0, 7.7, 3.7 Hz, 1H), 1.76 (dt, J=11.8, 8.2 Hz, 1H), 1.68-1.63 (m, 1H), 1.14-1.05 (m, 1H); ³¹P NMR (243 MHz, Chloroform-d) δ 152.83; MS (ESI), 870.63 [M −H]⁻.

### Synthesis of (2R, 3S, 4R, 5R)-5-((bis(4-methoxyphenyl)(phenyl)methoxy)methyl)-2-(2, 6-dioxo-3, 6-dihydropyrimidin-1(2H)-yl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-3-yl acetate

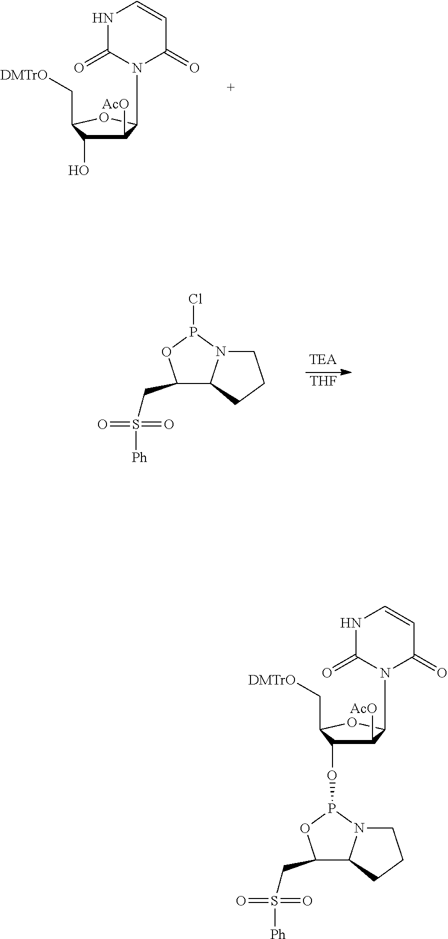

To a solution of dry [(2R, 3R, 5R)-5-[[bis(4-methoxyphenyl)-phenyl-methoxy]methyl]-2-(2, 4-dioxo-1H-pyrimidin-3-yl)-4-hydroxy-tetrahydrofuran-3-yl]acetate (1.9 g, 3.23 mmol) in THF (15 mL) was added triethylamine (0.99 mL, 7.1 mmol). The reaction flask was set in a water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.90Min THF, 5.38 mL, 4.84 mmol) was added fast dropwise. The water bath was removed. The cloudy reaction solution was stirred at rt for 2 hr. TLC and LCMS showed the reaction was almost complete. Stirred for another 1 hr. The reaction was quenched by water (29 L). Anhydrous MgSO₄ (387 mg) was added. The mixture was filtered through celite, and the filtrate was concentrated to afford the crude product as an off-white foam. The crude product was purified by normal phase column chromatography applying 40-100% EtOAc in hexanes (each mobile phase contained 1% triethylamine) as the gradient to afford the title compound as a white foam (1.02 g, 36.3% yield). ¹H NMR (600 MHz, Chloroform-d) δ 9.49 (s, 1H), 7.86 (d, J=7.8 Hz, 2H), 7.62 (t, J=7.5 Hz, 1H), 7.53-7.48 (m, 2H), 7.46 (d, J=7.9 Hz, 2H), 7.34 (dd, J=9.1, 2.9 Hz, 4H), 7.22 (t, J=7.6 Hz, 2H), 7.15 (t, J -7.3 Hz, 1H), 6.96 (d, J=7.6 Hz, 1H), 6.86 (d, J - 8.2 Hz, 1H), 6.77 (dd, J - 8.6, 5.8 Hz, 4H), 5.59 (d, J=7.5 Hz, 1H), 5.41 (t, J=7.5 Hz, 1H), 5.00 (q, J=6.0 Hz, 2H), 3.96 (td, J=8.1, 2.1 Hz, 1H), 3.76-3.73 (m, 6H), 3.62 (dq, J=11.5, 5.8 Hz, 1H), 3.57 (t, J=9.2 Hz, 1H), 3.33 (d, J=9.4 Hz, 1H), 3.32-3.22 (m, 3H), 2.74 (qd, J=9.4, 4.1 Hz, 1H), 1.98 (s, 3H), 1.77 (dp, J=12.5, 4.3 Hz, 1H), 1.73-1.65 (m, 1H), 1.61 (dt, J=9.3, 5.3 Hz, 1H), 1.11-1.01 (m, 1H); ³¹P NMR (243 MHz, Chloroform-d) δ 148.88; MS (ESI), 870.46 [M −H]⁻.

### Synthesis of N-(5-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-3-nitro-6-(4-nitrophenethoxy)pyridin-2-yl)acetamide

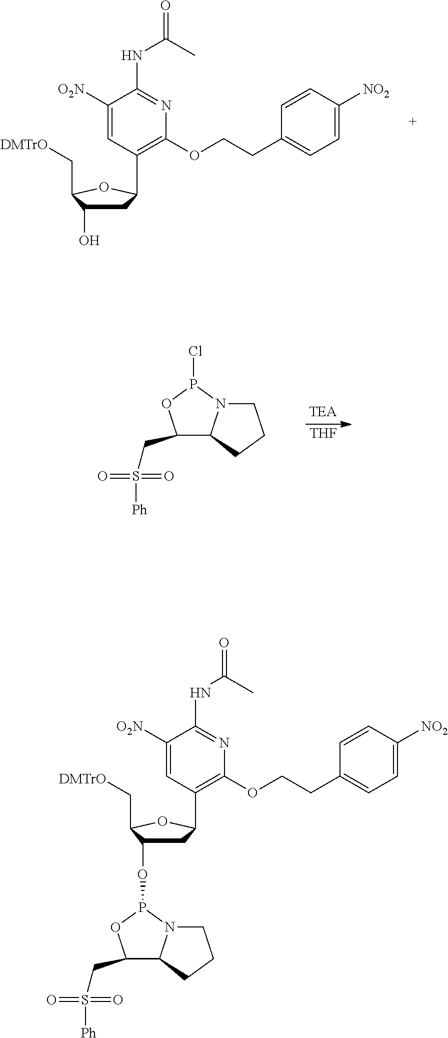

To a solution of dry N-[5-[(2R, 4R, 5R)-5-[[bis(4-methoxyphenyl)-phenyl-methoxy]methyl]-4-hydroxy-tetrahydrofuran-2-yl]-3-nitro-6-[2-(4-nitrophenyl)ethoxy]-2-pyridyl]acetamide (2.0 g, 2.62 mmol) in THF (15 mL) was added triethylamine (0.91 mL, 6.54 mmol). The reaction flask was set in a water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.90 M in THF, 5.23 mL, 4.71 mmol) was added dropwise. The water bath was removed. The cloudy reaction solution was stirred at rt for 4 hr 45 min. TLC and LCMS showed the reaction was complete. The reaction was quenched by water (24 L). Anhydrous MgSO₄ (314 mg) was added. The mixture was filtered through celite, and the filtrate was concentrated to afford the crude product as an off-white foam. The crude product was purified by normal phase column chromatography applying 20-70% EtOAc in hexanes (each mobile phase contained 10% triethylamine) as the gradient to afford the title compound as a light-yellow foam (1.897 g, 69.2% yield). ¹H NMR (600 MHz, Chloroform-d) δ 10.59 (s, 1H), 8.66 (s, 1H), 8.20 (d, J=8.6 Hz, 2H), 7.89 (d, J=7.5 Hz, 2H), 7.57 (tt, J=7.5, 1.4 Hz, 1H), 7.50-7.44 (m, 4H), 7.43-7.39 (m, 2H), 7.32 (dd, J=8.8, 3.8 Hz, 4H), 7.28 (t, J=7.6 Hz, 2H), 7.20 (t, J=7.2 Hz, 1H), 6.87-6.80 (m, 4H), 5.14-5.08 (m, 1H), 5.08-5.03 (m, 1H), 4.76-4.65 (m, 3H), 4.08 (q, J=3.6 Hz, 1H), 3.79 (s, 6H), 3.61 (dq, J=11.7, 5.9 Hz, 1H), 3.54 (ddd, J=14.5, 10.5, 7.5 Hz, 1H), 3.48 (dd, J=14.5, 7.0 Hz, 1H), 3.36 (dd, J=14.5, 5.3 Hz, 1H), 3.27 (dq, J=15.5, 6.3, 5.2 Hz, 3H), 3.22 (dd, J=10.2, 3.9 Hz, 1H), 3.13 (qd, J=10.3, 3.9 Hz, 1H), 2.46 (s, 3H), 2.38 (dd, J=13.0, 5.2 Hz, 1H), 1.90 (tdd, J=11.5, 7.6, 3.0 Hz, 1H), 1.84-1.73 (m, 2H), 1.64 (dt, J=12.0, 7.2 Hz, 1H), 1.17-1.07 (m, 1H); ³¹P NMR (243 MHz, Chloroform-d) δ 151.45; MS (ESI), 1086.17 [M+K]⁺.

### Synthesis of 3-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-5-bromopyrimidine-2, 4(1H, 3H)-dione

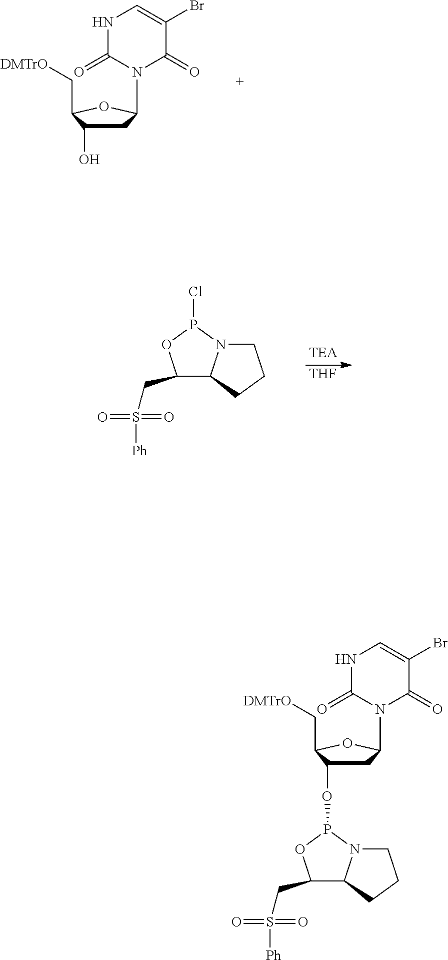

To a solution of dry 3-[(2R, 4R, 5R)-5-[[bis(4-methoxyphenyl)-phenyl-methoxy]methyl]-4-hydroxy-tetrahydrofuran-2-yl]-5-bromo-1H-pyrimidine-2, 4-dione (2.8 g, 4.59 mmol) in THF (21 mL) was added triethylamine (1.6 mL, 11.49 mmol). (3S, 3aS)-3-(benzenesulfonylmethyl)-1-chloro-3a, 4, 5, 6-tetrahydro-3H-pyrrolo[l, 2-c][1, 3, 2]oxazaphosphole (0.90M in THF, 9.19 mL, 8.27 mmol) was added dropwise. The resulting slurry was stirred at rt for 2 hr 45 min. TLC and LCMS showed the reaction was complete. The reaction was quenched by water (41 L). Anhydrous MgSO₄(551 mg) was added. The mixture was filtered through celite, and the filtrate was concentrated to afford the crude product as an off-white foam. The crude product was purified by normal phase column chromatography applying 45-100% EtOAc in hexanes (each mobile phase contained 1% triethylamine) as the gradient to afford the title compound as a brownish off-white foam (2.433 g, 59.3% yield). ¹H NMR (600 MHz, Chloroform-d) δ 7.87 (d, J=8.1 Hz, 2H), 7.63-7.58 (m, 1H), 7.50 (t, J=7.7 Hz, 2H), 7.47 (d, J=7.8 Hz, 2H), 7.35 (d, J=8.5 Hz, 4H), 7.24 (t, J=7.7 Hz, 2H), 7.16 (t, J=7.3 Hz, 1H), 6.93 (s, 1H), 6.82-6.76 (m, 4H), 6.70 (dd, J=8.5, 5.0 Hz, 1H), 4.91 (q, J=6.1 Hz, 1H), 4.85 (td, J=8.8, 4.4 Hz, 1H), 3.94 (td, J=6.2, 3.2 Hz, 1H), 3.751 (s, 3H), 3.748 (s, 3H), 3.60 (dp, J=11.7, 6.5, 6.1 Hz, 1H), 3.43-3.33 (m, 3H), 3.33-3.26 (m, 2H), 2.96 (qd, J=10.0, 4.0 Hz, 1H), 2.86 (ddd, J=13.3, 8.2, 5.0 Hz, 1H), 2.26 (ddd, J=13.7, 8.5, 5.5 Hz, 1H), 1.82 (ddq, J=11.9, 7.7, 3.9, 3.3 Hz, 1H), 1.72 (dt, J=14.9, 9.1 Hz, 1H), 1.60 (qd, J=7.5, 2.6 Hz, 1H), 1.16-1.01 (m, 2H); “P NMR (243 MHz, Chloroform-d) δ 150.19; MS (ESI), 890.46/892.45 [M−H]⁻.

### Synthesis of N, N′-(9-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-9H-purine-2, 6-diyl)bis(2-methylpropanamide)

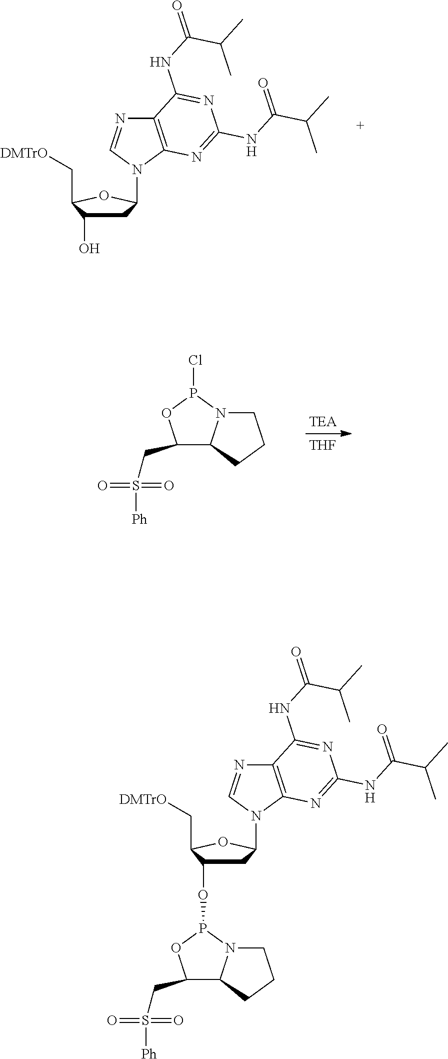

To a solution of dry N-[9-[(2R, 4R, 5R)-5-[[bis(4-methoxyphenyl)-phenyl-methoxy]methyl]-4-hydroxy-tetrahydrofuran-2-yl]-2-(2-methylpropanoylamino)purin-6-yl]-2-methyl-propanamide (3.25 g, 4.59 mmol) in THF (24.4 mL) was added triethylamine (1.6 mL, 11.46 mmol). The flask was set in a water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.90Min THF, 9.17 mL, 8.25 mmol) was added dropwise. The water bath was removed. The resulting slurry was stirred at rt for 2.5 hr. TLC and LCMS showed the reaction was complete. The reaction was quenched by water (41 μL). Anhydrous MgSO₄ (551 mg) was added. The mixture was filtered through celite, and the filtrate was concentrated to afford the crude product as an off-white foam. The crude product was purified by normal phase column chromatography applying 30-100% EtOAc in hexanes (each mobile phase contained I % triethylamine) as the gradient to afford the title compound as a white foam (2.52 g, 55.4% yield). ¹H NMR (600 MHz, Chloroform-d) δ 8.65 (s, 1H), 8.27 (s, 1H), 8.05 (s, 1H), 7.92-7.88 (m, 2H), 7.57 (t, J=7.5 Hz, 1H), 7.48 (t, J=7.7 Hz, 2H), 7.39 (d, J=7.6 Hz, 2H), 7.28 (d, J=8.6 Hz, 4H), 7.23 (t, J=7.5 Hz, 2H), 7.19 (t, J=7.2 Hz, 1H), 6.80-6.75 (m, 4H), 6.38 (dd, J=7.9, 5.8 Hz, 1H), 5.03 (q, J=6.0 Hz, 1H), 4.88 (ddt, J=8.8, 5.7, 2.5 Hz, 1H), 4.15-4.13 (m, 1H), 3.76 (s, 6H), 3.63 (dq, J=11.6, 6.1 Hz, 1H), 3.53-3.43 (m, 2H), 3.40-3.30 (m, 2H), 3.13 (qd, J=9.8, 3.7 Hz, 2H), 2.88 (ddd, J=13.8, 8.0, 6.1 Hz, 1H), 2.63 (ddd, J=13.5, 5.8, 2.4 Hz, 1H), 2.14 (s, 2H), 1.88 (ddq, J=11.7, 7.6, 3.7, 3.1 Hz, 1H), 1.79 (tt, J=12.0, 7.7 Hz, 1H), 1.64 (qd, J=7.6, 2.4 Hz, 1H), 1.29 (d, J=6.8 Hz, 6H), 1.18 (dd, J=11.4, 6.8 Hz, 6H), 1.14-1.07 (m, 1H); ³¹P NMR (243 MHz, Chloroform-d) δ 152.69; MS (ESI), 992.72 [M+H]⁺.

### Synthesis of 1-(9-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-8-oxo-8, 9-dihydro-7H-purin-6-yl)-3-phenylurea

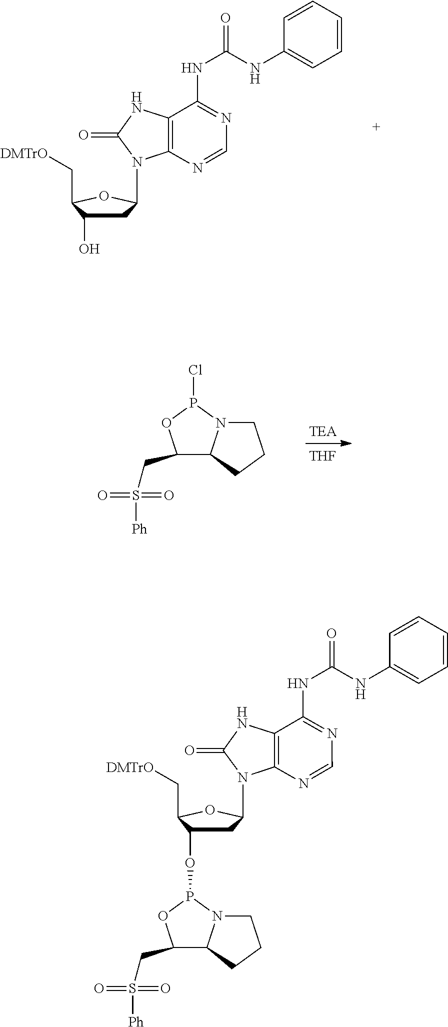

To a solution of dry 1-[9-[(2R, 4R, 5R)-5-[[bis(4-methoxyphenyl)-phenyl-methoxy]methyl]-4-hydroxy-tetrahydrofuran-2-yl]-8-oxo-7H-purin-6-yl]-3-phenyl-urea (3.95 g, 5.74 mmol) in THF (30 mL) was added triethylamine (2.0 mL, 14.34 mmol). The flask was set in a water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.90Min THF, 11.47 mL, 10.32 mmol) was added dropwise. The water bath was removed. The resulting slurry was stirred at rt for 3 hr. TLC and LCMS showed the reaction was complete. The reaction was quenched by water (52 μL). Anhydrous MgSO₄ (688 mg) was added. The mixture was filtered through celite, and the filtrate was concentrated to afford the crude product as a light-yellow foam. The crude product was purified by normal phase column chromatography applying 0-100% acetonitrile in EtOAc (each mobile phase contained 1% triethylamine) as the gradient to afford the title compound as an off-white foam (1.70 g, 30.6% yield). ¹H NMR (600 MHz, Chloroform-d) δ 11.67 (s, 2H), 10.62 (s, 1H), 8.27 (s, 1H), 7.90 (d, J=7.9 Hz, 2H), 7.59 (t, J=7.8 Hz, 1H), 7.51 (dt, J=26.1, 8.9 Hz, 6H), 7.43 (d, J=7.8 Hz, 2H), 7.31 (d, J=8.3 Hz, 4H), 7.20 (t, J=7.6 Hz, 3H), 7.16 (d, J=7.4 Hz, 1H), 6.74 (t, J=9.1 Hz, 4H), 6.38-6.32 (m, 1H), 5.10 (q, J=6.2 Hz, 2H), 4.08-4.02 (m, 1H), 3.73 (s, 3H), 3.72 (s, 3H), 3.71-3.66 (m, 1H), 3.47 (dd, J=14.6, 6.9 Hz, 2H), 3.42-3.30 (m, 3H), 3.26 (t, J=8.2 Hz, 1H), 3.07-2.99 (m, 1H), 2.30 (dt, J=12.7, 5.4 Hz, 1H), 1.87 (t, J=9.2 Hz, 1H), 1.78 (p, J=10.0, 9.4 Hz, 1H), 1.67 (dt, J=13.1, 6.1 Hz, 1H), 1.18-1.08 (m, 1H); ³¹P NMR (243 MHz, Chloroform-d) δ 149.65; MS (ESI), 970.45 [M−H]⁻.

### Synthesis of 3-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-4-thioxo-3, 4-dihydropyrimidin-2(1H)-one

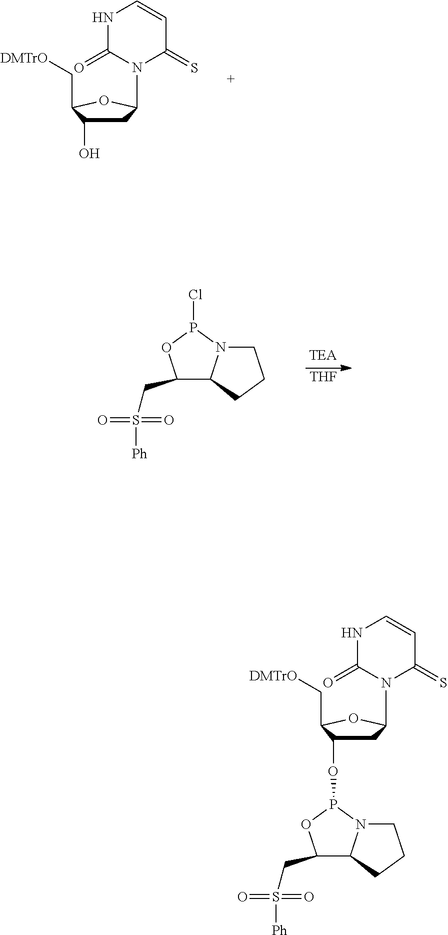

To a solution of dry 3-[(2R, 4R, 5R)-5-[[bis(4-methoxyphenyl)-phenyl-methoxy]methyl]-4-hydroxy-tetrahydrofuran-2-yl]-4-thioxo-1H-pyrimidin-2-one (2.2 g, 4.02 mmol) in THF (16.5 mL) was added triethylamine (1.4 mL, 10.06 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75 M in THF, 8.05 mL, 6.04 mmol) was added dropwise. The ice/water bath was removed. The resulting slurry was stirred at rt for 5 hr. TLC and LCMS showed the reaction was complete. The reaction was quenched by water (36 L). Anhydrous MgSO₄ (483 mg) was added. The mixture was filtered through celite, and the filtrate was concentrated to afford the crude product as an off-white foam. The crude product was purified by normal phase column chromatography applying 20-100% EtOAc in hexanes (each mobile phase contained 1% triethylamine) as the gradient to afford the title compound as a light-yellow foam (1.35 g, 40.4% yield). ¹H NMR (600 MHz, Chloroform-d) δ 7.86 (d, J=7.8 Hz, 2H), 7.61 (p, J=7.0 Hz, 2H), 7.51 (t, J=7.7 Hz, 2H), 7.46 (d, J=7.6 Hz, 2H), 7.34 (d, J=8.3 Hz, 3H), 7.23 (t, J=7.8 Hz, 2H), 7.16 (d, J=7.7 Hz, 1H), 6.79 (d, J=8.4 Hz, 4H), 6.75 (d, J=8.4 Hz, 1H), 6.43 (d, J=7.3 Hz, 1H), 6.38 (d, J=7.3 Hz, 1H), 4.95-4.90 (m, 1H), 4.86 (p, J=7.8 Hz, 1H), 3.96 (dd, J=7.6, 4.2 Hz, 1H), 3.75 (s, 6H), 3.60 (dq, J=13.6, 6.5 Hz, 1H), 3.42-3.26 (m, 5H), 2.97-2.90 (m, 1H), 2.78 (p, J=6.5 Hz, 1H), 2.28 (dt, J=13.8, 7.1 Hz, 1H), 1.85-1.79 (m, 1H), 1.72 (q, J=10.5, 9.8 Hz, 1H), 1.61 (p, J=6.3 Hz, 1H), 1.58-1.49 (m, 1H); “¹P NMR (243 MHz, Chloroform-d) δ 149.15; MS (ESI), 828.38 [M−H]⁻.

### Synthesis of (2R, 3S, 4R, 5R)-2-(6-benzamido-7-benzoyl-8-oxo-7, 8-dihydro-9H-purin-9-yl)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-3-yl benzoate

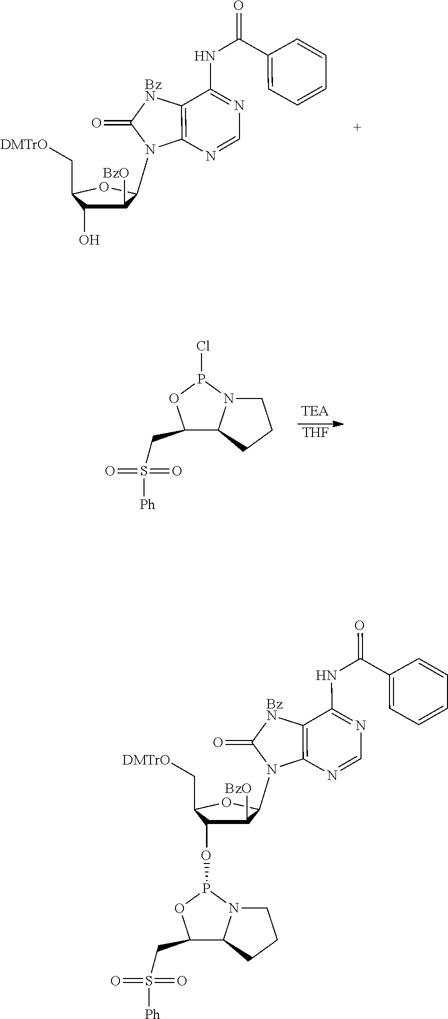

To a solution of dry [(2R, 3R, 5R)-2-(6-benzamido-7-benzoyl-8-oxo-purin-9-yl)-5-[[bis(4-methoxyphenyl)-phenyl-methoxy]methyl]-4-hydroxy-tetrahydrofuran-3-yl]benzoate (2.5 g, 2.78 mmol) in THF (19 mL) was added triethylamine (0.97 mL, 6.96 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75 M in THF, 6.31 mL, 4.73 mmol) was added dropwise. The ice/water bath was removed. The resulting slurry was stirred at rt for 1.5 hr. TLC and LCMS showed the reaction was complete. The reaction was quenched by water (25 μL). Anhydrous MgSO₄ (334 mg) was added. The mixture was filtered through celite, and the filtrate was concentrated to afford the crude product as an off-white foam. The crude product was purified by normal phase column chromatography applying 15-70% EtOAc in hexanes (each mobile phase contained 1% triethylamine) as the gradient to afford the title compound as an off-white foam (1.54 g, 46.9% yield). ¹H NMR (600 MHz, Chloroform-d) δ 8.26 (s, 1H), 7.94-7.88 (m, 2H), 7.87-7.81 (m, 2H), 7.78 (dd, J=17.2, 7.8 Hz, 3H), 7.68 (d, J=7.8 Hz, 2H), 7.60 (t, J=7.7 Hz, 1H), 7.54 (t, J=7.8 Hz, 1H), 7.49 (t, J=8.0 Hz, 1H), 7.46-7.39 (m, 7H), 7.33 (dt, J=6.4, 2.8 Hz, 3H), 7.28 (q, J=6.6, 4.7 Hz, 4H), 7.15 (t, J=7.7 Hz, 2H), 7.11 (t, J=7.3 Hz, 1H), 6.80-6.71 (m, 4H), 6.66 (d, J=7.5 Hz, 1H), 5.77 (t, J=7.1 Hz, 1H), 5.16 (q, J =8.1 Hz, 1H), 4.89 (q, J=6.4 Hz, 1H), 4.15-4.10 (m, 1H), 3.74 (s, 6H), 3.57 (q, J=8.8, 8.1 Hz, 2H), 3.43 (d, J=10.2 Hz, 1H), 3.26 (qd, J=14.3, 6.2 Hz, 3H), 2.82-2.75 (m, 1H), 1.79-1.64 (m, 2H), 1.63-1.53 (m, 2H); ³¹P NMR (243 MHz, Chloroform-d) δ 153.71; MS (ESI), 1179.43 [M−H]⁻.

### Synthesis of N-(9-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-8-thioxo-8, 9-dihydro-7H-purin-6-yl)benzamide

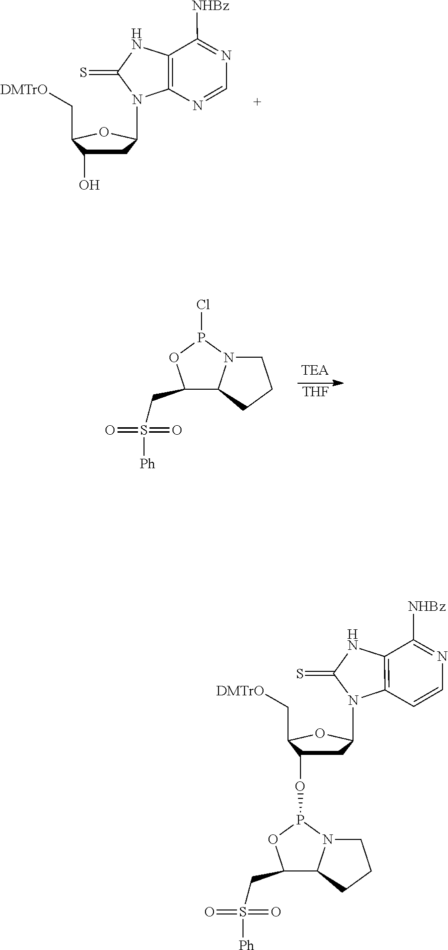

To a solution of dry N-[9-[(2R, 4R, 5R)-5-[[bis(4-methoxyphenyl)-phenyl-methoxy]methyl]-4-hydroxy-tetrahydrofuran-2-yl]-8-thioxo-7H-purin-6-yl]benzamide (2.5 g, 3.62 mmol) in THF (19 mL) was added triethylamine (1.26 mL, 9.06 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75 M in THF, 8.22 mL, 6.16 mmol) was added dropwise. The ice/water bath was removed. The resulting slurry was stirred at rt for 1.5 hr. TLC and LCMS showed the reaction was complete. The reaction was quenched by water (33 L). Anhydrous MgSO₄ (435 mg) was added. The mixture was filtered through celite, and the filtrate was concentrated to afford the crude product as an off-white foam. The crude product was purified by normal phase column chromatography applying 20-100% EtOAc in hexanes (each mobile phase contained 2.5% triethylamine) as the gradient to afford the title compound as a yellow foam (2.96 g, 84.0% yield). ¹H NMR (600 MHz, Chloroform-d) δ 8.67 (s, 2H), 8.24 (d, J=2.5 Hz, 1H), 7.95 (d, J=7.8 Hz, 2H), 7.89 (d, J=7.8 Hz, 2H), 7.66 (t, J=7.7 Hz, 1H), 7.61 (t, J=7.7 Hz, 1H), 7.56 (t, J=7.6 Hz, 2H), 7.52 (t, J=7.9 Hz, 2H), 7.42 (d, J=7.8 Hz, 2H), 7.30 (d, J=8.1 Hz, 4H), 7.22 (t, J=7.9 Hz, 2H), 7.16 (t, J=7.5 Hz, 1H), 6.98 (t, J=7.6 Hz, 1H), 6.79-6.73 (m, 4H), 5.25-5.18 (m, 1H), 5.08 (q, J=6.6 Hz, 1H), 4.11 (dd, J=7.9, 4.9 Hz, 1H), 3.76 (s, 6H), 3.70 (t, J=8.0 Hz, 1H), 3.55-3.45 (m, 2H), 3.45-3.40 (m, 1H), 3.37 (dt, J=13.7, 6.6 Hz, 2H), 3.32-3.26 (m, 1H), 3.05 (d, J=10.3 Hz, 1H), 2.39 (tt, J=7.3, 3.0 Hz, 1H), 1.90 (ddt, J=15.9, 11.6, 5.8 Hz, 1H), 1.78 (q, J=9.9 Hz, 1H), 1.68 (dt, J=12.6, 6.0 Hz, 1H), 1.12 (q, J=10.3 Hz, 1H); ³¹P NMR (243 MHz, Chloroform-d) δ 149.85; MS (ESI), 971.40 [M−H]⁻.

### Synthesis of (E)-N′-(1-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-5-methyl-4-oxo-1, 4-dihydropyrimidin-2-yl)-N, N-dimethylformimidamide

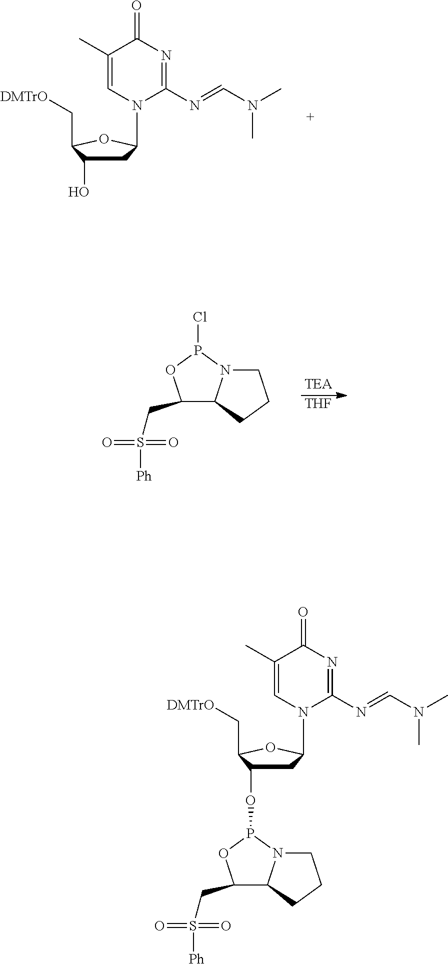

To a solution of dry 5′—ODMTr-N-DMF-5-methyl-Isocytidine (20 g, 33.41 mmol) in THF (300 mL) was added triethylamine (11.64 mL, 83.52 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75 M in THF, 75.72 mL, 56.79 mmol) was added dropwise. The ice/water bath was removed. The slurry was stirred at rt for 6.5 hr. TLC and LCMS showed the reaction was complete. The reaction was quenched by water (301 μL). Anhydrous MgSO₄ (4.0 g) was added. The mixture was filtered through celite, and the filtrate was concentrated to afford the crude product as an off-white foam. The crude product was purified by normal phase column chromatography first applying 20-100% EtOAc in hexanes (each mobile phase contained 2.5% triethylamine) then applying 0-90% acetonitrile in EtOAc (each mobile phase contained 5% triethylamine) as the gradient to afford the title compound as an off-white foam (21.9 g, 74.3% yield). ¹H NMR (600 MHz, Chloroform-d) δ 8.82 (s, 1H), 7.86 (d, J=7.7 Hz, 2H), 7.66 (s, 1H), 7.58 (t, J=7.8 Hz, 1H), 7.47 (t, J=7.8 Hz, 2H), 7.44-7.39 (m, 2H), 7.33-7.26 (m, 6H), 7.22 (t, J=7.4 Hz, 1H), 6.83 (d, J=8.3 Hz, 4H), 6.81-6.75 (m, 1H), 4.97 (q, J=6.3 Hz, 1H), 4.81-4.75 (m, 1H), 3.99 (q, J=2.5 Hz, 1H), 3.77 (s, 6H), 3.57 (dq, J =12.3, 6.2 Hz, 1H), 3.52-3.39 (m, 3H), 3.33 (dd, J=14.6, 5.5 Hz, 1H), 3.26 (dd, J=10.6, 2.5 Hz, 1H), 3.14 (s, 3H), 3.10 (s, 3H), 2.50-2.44 (m, 1H), 2.26 (dt, J=14.3, 7.4 Hz, 1H), 1.92-1.85 (m, 1H), 1.83-1.70 (m, 2H), 1.64 (q, J=5.7 Hz, 1H), 1.61 (s, 3H), 1.09 (p, J=10.4 Hz, 1H); ³¹P NMR (243 MHz, Chloroform-d) δ 152.53; MS (ESI), 882.59 [M+H]⁺.

### Synthesis of 3-((2R, 4S, 5R)-5-((bis(4-methoxyphenyl)(phenyl)methoxy)methyl)-4-(((1R, 3R, 3aR)-3-((phenylsulfonyl)methyl)tetrahydro-1H, 3H-pyrrolo[1, 2-c][1, 3, 2]oxazaphosphol-1-yl)oxy)tetrahydrofuran-2-yl)pyrimidine-2, 4(1H, 3H)-dione

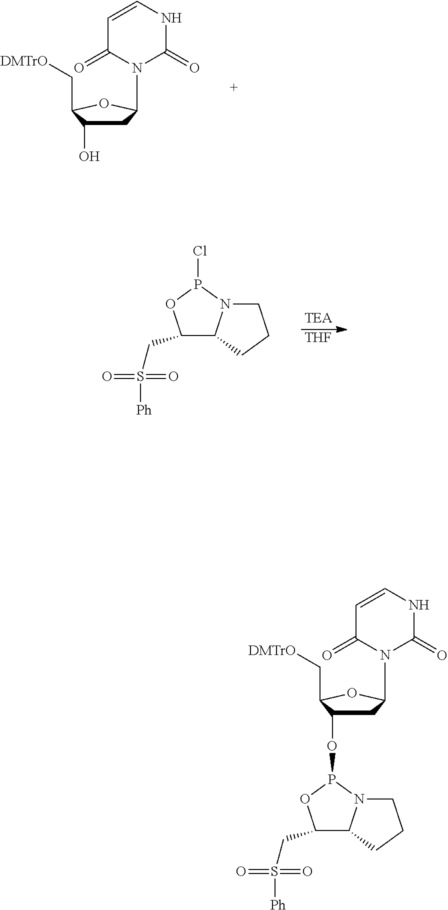

To a solution of dry 3-[(2R, 4R, 5R)-5-[[bis(4-methoxyphenyl)-phenyl-methoxy]methyl]-4-hydroxy-tetrahydrofuran-2-yl]-1H-pyrimidine-2, 4-dione (5.0 g, 9.42 mmol) in THF (37.5 mL) was added triethylamine (3.28 mL, 23.56 mmol). The reaction flask was set in an ice/water bath. (3R, 3aR)-3-(benzenesulfonylmethyl)-1-chloro-3a, 4, 5, 6-tetrahydro-3H-pyrrolo[1, 2-c][1, 3, 2]oxazaphosphole (0.8M in THF, 18.85 mL, 15.08 mmol) was added dropwise. The ice/water bath was removed. The resulting slurry was stirred at rt for 5 hr. TLC and LCMS showed the reaction was complete. The reaction was quenched by water (85 μL). Anhydrous MgSO₄ (1.13 g) was added. The mixture was filtered through celite, and the filtrate was concentrated to afford the crude product as an off-white foam. The crude product was purified by normal phase column chromatography applying 20-100% EtOAc in hexanes (each mobile phase contained 2.5% triethylamine) as the gradient to afford the title compound as a white foam (6.3 g, 82.1% yield). ¹H NMR (600 MHz, Chloroform-d) δ 9.31 (s, 1H), 7.91 (d, J=7.8 Hz, 2H), 7.63 (t, J=7.7 Hz, 1H), 7.54 (t, J=7.8 Hz, 2H), 7.46 (d, J=7.8 Hz, 2H), 7.34 (d, J=8.3 Hz, 4H), 7.23 (t, J=7.8 Hz, 2H), 7.16 (t, J=7.6 Hz, 1H), 6.78 (ddt, J=6.7, 4.5, 2.5 Hz, 4H), 6.68 (d, J=8.0 Hz, 2H), 5.55 (d, J=7.4 Hz, 1H), 5.00 (q, J=6.2 Hz, 1H), 4.81 (quintuplet, J=6.3 Hz, 1H), 4.02 (q, J=5.6 Hz, 1H), 3.75 (s, 6H), 3.60 (quintuplet, J=7.9 Hz, 1H), 3.48 (dd, J=14.8, 7.6 Hz, 1H), 3.43-3.34 (m, 3H), 3.34-3.28 (m, 1H), 3.10 (q, J=9.12 Hz, 1H), 2.84-2.77 (m, 1H), 2.14 (dt, J=14.0, 7.2 Hz, 1H), 1.83 (d, J=9.6 Hz, 1H), 1.79-1.70 (m, 1H), 1.60 (quintuplet, J=6.2 Hz, 1H), 1.09 (h, J=10.1, 9.5 Hz, 1H); ³¹P NMR (243 MHz, Chloroform-d) δ 155.86; MS (ESI), 812.46 [M−H]⁻.

### Synthesis of 3-((2R, 3R, 4R, 5R)-5-((bis(4-methoxyphenyl)(phenyl)methoxy)methyl)-3-((tert-butyldimethylsilyl)oxy)-4-(((IS, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)pyrimidine-2, 4(1H, 3H)-dione

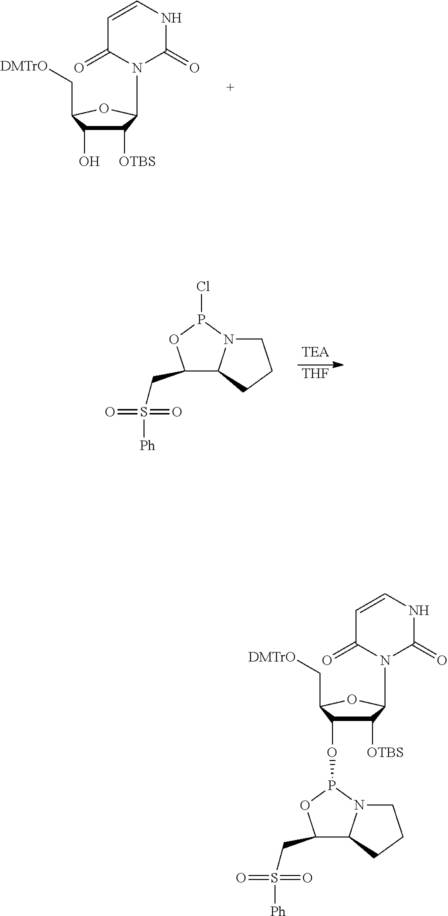

To a solution of dry 3-[(2R, 4S, 5R)-5-[[bis(4-methoxyphenyl)-phenyl-methoxy]methyl]-3-[tert-butyl(dimethyl)silyl]oxy-4-hydroxy-tetrahydrofuran-2-yl]-1H-pyrimidine-2. 4-dione (5.0 g, 7.57 mmol) in THF (37.5 mL) was added triethylamine (2.64 mL, 18.92 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75M in THF, 17.15 mL, 12.86 mmol) was added dropwise. The ice/water bath was removed. The resulting slurry was stirred at rt for 4 h 45 min. The reaction was quenched by water (68 μL). Anhydrous MgSO₄ (908 mg) was added. The mixture was filtered through celite, and the filtrate was concentrated. The resulting crude product was purified by normal phase column chromatography applying 20-100% EtOAc in hexanes (each mobile phase contained 2.5% triethylamine) as the gradient to afford the title compound as a white foam (4.206 g, 58.9% yield). ¹H NMR (600 MHz, Chloroform-d) δ 9.51 (s, 1H), 7.84 (d, J=7.7 Hz, 2H), 7.57 (t, J=7.7 Hz, 1H), 7.51 (d, J=8.2 Hz, 2H), 7.45 (t, J=7.9 Hz, 2H), 7.40-7.32 (m, 4H), 7.23 (t, J=7.8 Hz, 2H), 7.16 (t, J=7.6 Hz, 1H), 6.79 (d, J=8.3 Hz, 4H), 6.27 (d, J=3.1 Hz, 1H), 6.20 (d, J=7.5 Hz, 1H), 5.42 (d, J=7.5 Hz, 1H), 5.00 (s, 1H), 4.88 (q, J=6.5 Hz, 1H), 4.63 (dt, J=11.4, 5.5 Hz, 1H), 3.98-3.93 (m, 1H), 3.75 (s, 6H), 3.58 (q, J=7.2, 6.6 Hz, 1H), 3.44-3.29 (m, 4H), 3.08 (dd, J=10.4, 5.0 Hz, 1H), 3.00-2.92 (m, 1H), 1.81 (t, J=9.9 Hz, 1H), 1.70 (q, J=10.8, 9.8 Hz, 1H), 1.59 (q, J=6.9, 6.2 Hz, 1H), 1.10-1.00 (m, 1H), 0.87 (s, 9H), 0.04 (s, 3H), 0.00 (s, 3H); ³¹P NMR (243 MHz, Chloroform-d) δ 151.37; MS (ESI), 942.40 [M−H]⁻.

### Synthesis of 3-((2R, 3R. 4R, 5R)-5-((bis(4-methoxyphenyl)(phenyl)methoxy)methyl)-4-((tert-butyldimethylsilyl)oxy)-3-(((IS, 3S, 3aS)-3-((phenylsulfonyl)methyl)tetrahydro-1H, 3H-pyrrolo[l, 2-c][1, 3, 2]oxazaphosphol-1-yl)oxy)tetrahydrofuran-2-yl)pyrimidine-2, 4(1H, 3H)-dione

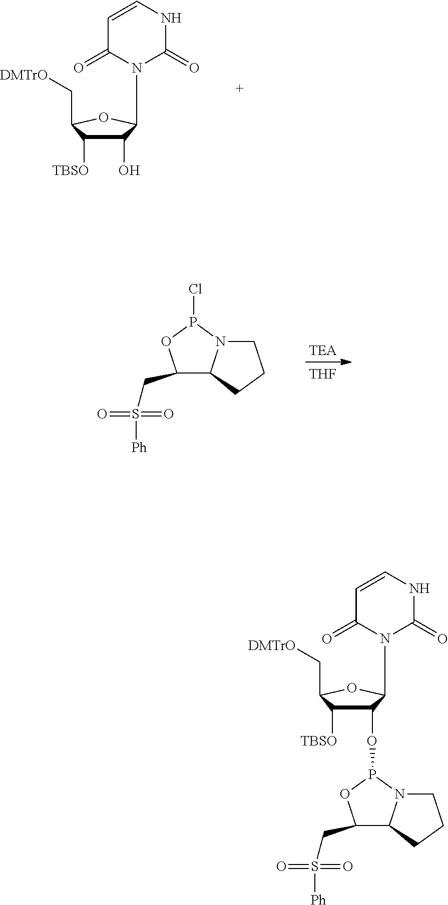

To a solution of dry 3-[(2R, 4R, 5R)-5-[[bis(4-methoxyphenyl)-phenyl-methoxy]methyl]-4-[tert-butyl(dimethyl)silyl]oxy-3-hydroxy-tetrahydrofuran-2-yl]-1H-pyrimidine-2, 4-dione (5.0 g, 7.57 mmol) in THF (37.5 mL) was added triethylamine (2.64 mL, 18.92 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75M in THF, 18.16 mL, 13.62 mmol) was added dropwise. The ice/water bath was removed. The resulting slurry was stirred at rt for 4 hr. TLC and LCMS showed the reaction was complete. The reaction was quenched by water (68 μL). Anhydrous MgSO₄(908 mg) was added. The mixture was filtered through celite, and the filtrate was concentrated. The resulting crude product was purified by normal phase column chromatography applying 20-95% EtOAc in hexanes (each mobile phase contained 2.5% triethylamine) as the gradient to afford the title compound as a brownish off-white foam (3.587 g, 50.2% yield). ¹H NMR (600 MHz, Chloroform-d) δ 9.35 (s, 1H), 7.97-7.93 (m, 2H), 7.66-7.61 (m, 1H), 7.56 (dd, J=8.4, 7.2 Hz, 2H), 7.48-7.44 (m, 2H), 7.38-7.31 (m, 4H), 7.23 (t, J=7.7 Hz, 2H), 7.18-7.12 (m, 1H), 6.86 (d, J=7.7 Hz, 1H), 6.78 (dt, J=9.4, 2.2 Hz, 4H), 6.28 (d, J=3.6 Hz, 1H), 5.58 (d, J=7.6 Hz, 1H), 5.05-4.97 (m, 2H), 4.44 (t, J=6.4 Hz, 1H), 4.03 (td, J=6.1, 3.5 Hz, 1H), 3.75 (s, 6H), 3.72-3.67 (m, 1H), 3.47-3.37 (m, 3H), 3.33 (dd, J=14.4, 5.9 Hz, 1H), 3.13 (dd, J=10.4, 5.8 Hz, 1H), 2.97 (tt, J=10.0, 6.5 Hz, 1H), 1.76-1.64 (m, 3H), 1.18 (dq, J=11.7, 8.3 Hz, 1H), 0.78 (s, 9H), 0.00 (s, 3H), −0.08 (s, 3H); ³¹P NMR (243 MHz, Chloroform-d) δ 148.50; MS (ESI), 942.40 [M−H]⁻.

### Synthesis of 9-((2R, 4S, 5R)-5-((bis(4-methoxyphenyl)(phenyl)methoxy)methyl)-4-(((1R, 3R, 3aR)-3-((phenylsulfonyl)methyl)tetrahydro-1H, 3H-pyrrolo[1, 2-c][1, 3, 2]oxazaphosphol-1-yl)oxy)tetrahydrofuran-2-yl)-6-oxo-N, N-diphenyl-6, 9-dihydro-1H-purine-1-carboxamide

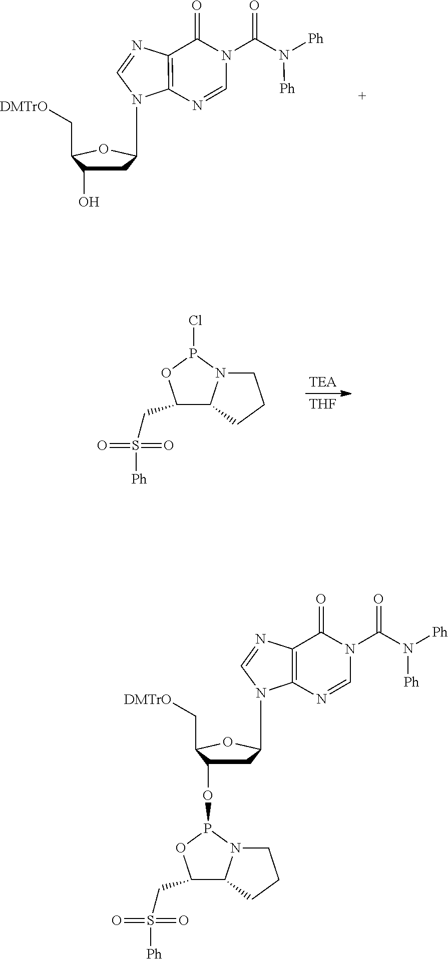

To a solution of dry 9-[(2R, 4R, 5R)-5-[[bis(4-methoxyphenyl)-phenyl-methoxy]methyl]-4-hydroxy-tetrahydrofuran-2-yl]-6-oxo-N, N-diphenyl-purine-1-carboxamide (13.06 g, 17.42 mmol) in THF (98 mL) was added triethylamine (6.07 mL, 43.54 mmol). The reaction flask was set in an ice/water bath. (3R, 3aR)-3-(benzenesulfonylmethyl)-1-chloro-3a, 4, 5, 6-tetrahydro-3H-pyrrolo[1, 2-c][1, 3, 2]oxazaphosphole (0.80 M in THF, 39.19 mL, 31.35 mmol) was added dropwise. The ice/water bath was removed. The resulting slurry was stirred at rt for 3 hr 45 min. TLC and LCMS showed the reaction was complete. The reaction was quenched by water (157 L). Anhydrous MgSO₄ (2.08 g) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a white foam (14.7 g, 81.7% yield). ¹H NMR (600 MHz, Chloroform-d) δ 8.05 (s, 1H), 7.90 (t, J=12.5 Hz, 2H), 7.83 (s, 1H), 7.59 (s, 1H), 7.57-7.46 (m, 2H), 7.44 (s, 2H), 7.42-7.33 (m, 5H), 7.32-7.23 (m, 6H), 7.21 (t, J=7.4 Hz, 2H), 7.18 (s, 2H), 7.11 (s, 1H), 7.05 (s, 1H), 6.83-6.77 (m, 4H), 6.24 (dd, J=7.3, 6.0 Hz, 1H), 5.09 (dq, J=7.8, 4.6 Hz, 1H), 4.86-4.77 (m, 1H), 4.24-4.16 (m, 1H), 3.782 (s, 3H), 3.781 (s, 3H), 3.69-3.63 (m, 1H), 3.55-3.44 (m, 2H), 3.42-3.33 (m, 2H), 3.29 (dd, J=10.5, 3.9 Hz, 1H), 3.19 (tdd, J=10.2, 8.7, 4.0 Hz, 1H), 2.64 (dt, J=13.5, 6.7 Hz, 1H), 2.53-2.39 (m, 1H), 1.92-1.86 (m, 1H), 1.85-1.75 (m, 1H), 1.65-1.60 (m, 1H), 1.13 (dtd, J=11.7, 10.3, 8.6 Hz, 1H); “P NMR (243 MHz, Chloroform-d) δ 156.79 (minor), 156.34 (major); minor: major=47: 53; MS (ESI), 1033.10 [M+H]⁺.

### Synthesis of 9-((2R, 4S, 5R)-5-((bis(4-methoxyphenyl)(phenyl)methoxy)methyl)-4-(((1R, 3R, 3aR)-3-((phenylsulfonyl)methyl)tetrahydro-1H, 3H-pyrrolo[1, 2-c][1, 3, 2]oxazaphosphol-1-yl)oxy)tetrahydrofuran-2-yl)-9H-purin-6-yl diphenylcarbamate

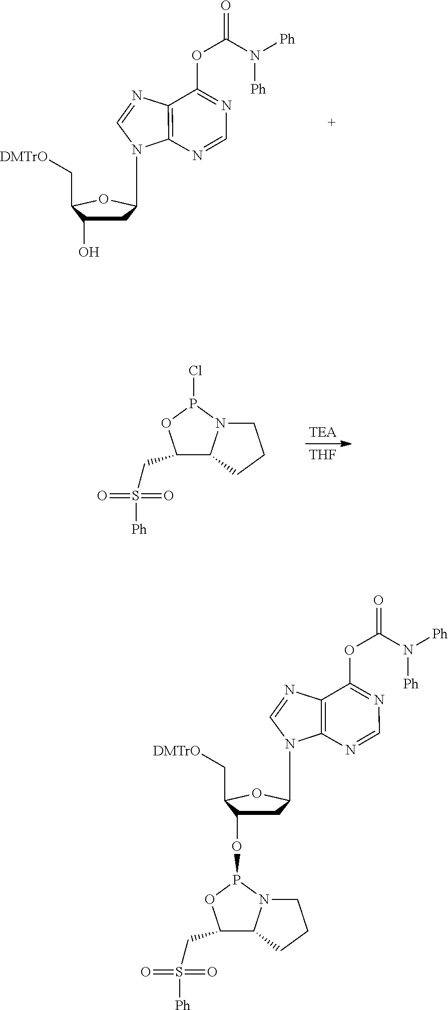

To a solution of dry [9-[(2R, 4R, 5R)-5-[[bis(4-methoxyphenyl)-phenyl-methoxy]methyl]-4-hydroxy-tetrahydrofuran-2-yl]purin-6-yl]N, N-diphenylcarbamate (3.6 g, 4.8 mmol) in THF (27 mL) was added triethylamine (1.67 mL, 12.0 mmol). The reaction flask was set in an ice/water bath. (3R, 3aR)-3-(benzenesulfonylmethyl)-1-chloro-3a, 4, 5, 6-tetrahydro-3H-pyrrolo[1, 2-c][1, 3, 2]oxazaphosphole (0.80M in THF, 10.8 mL, 8.64 mmol) was added dropwise. The ice/water bath was removed. The resulting slurry was stirred at rt for 2.5 hr. TLC and LCMS showed the reaction was complete. The reaction was quenched by water (43 μL). Anhydrous MgSO₄(576 mg) was added. The mixture was filtered through celite, and the filtrate was concentrated. The resulting crude product was purified by normal phase column chromatography applying 20-80% EtOAc in hexanes (each mobile phase contained 2.5% triethylamine) as the gradient to afford the title compound as a brownish off-white foam (3.94 g, 79.4% yield). ¹H NMR (600 MHz, Chloroform-d) δ 8.62 (s, 1H), 8.28 (s, 1H), 7.93-7.89 (m, 2H), 7.60-7.56 (m, 1H), 7.53-7.48 (m, 2H), 7.48-7.41 (m, 4H), 7.41-7.38 (m, 2H), 7.35 (t, J=7.9 Hz, 4H), 7.32-7.27 (m, 4H), 7.26-7.20 (m, 4H), 7.20-7.17 (m, 1H), 6.82-6.75 (m, 4H), 6.44 (dd, J=7.3, 6.0 Hz, 1H), 5.10 (ddd, J=7.7, 5.8, 4.5 Hz, 1H), 4.86 (ddt, J=8.8, 6.1, 3.1 Hz, 1H), 4.27 (q, J=4.1 Hz, 1H), 3.749 (s, 3H), 3.747 (s, 3H), 3.70-3.64 (m, 1H), 3.55-3.46 (m, 2H), 3.44 (dd, J=10.4. 4.7 Hz, 1H), 3.39 (dd, J=14.5, 4.4 Hz, 1H), 3.34 (dd, J=10.4, 4.3 Hz, 1H), 3.20 (tdd, J=10.1, 8.7, 4.0 Hz, 1H), 2.91 (ddd, J=13.4, 7.4, 6.0 Hz, 1H), 2.56 (ddd, J=13.6, 6.0, 3.3 Hz, 1H), 1.90 (tdd, J=11.8, 9.4, 5.6 Hz, 1H), 1.85-1.77 (m, 1H), 1.68-1.61 (m, 1H), 1.14 (dtd, J=11.6, 10.2, 8.4 Hz, 1H); ³¹P NMR (243 MHz, Chloroform-d) δ 156.38; MS (ESI), 1033.00 [M+H]⁺.

### Synthesis of 9-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-6-oxo-N, N-diphenyl-6, 9-dihydro-1H-purine-1-carboxamide

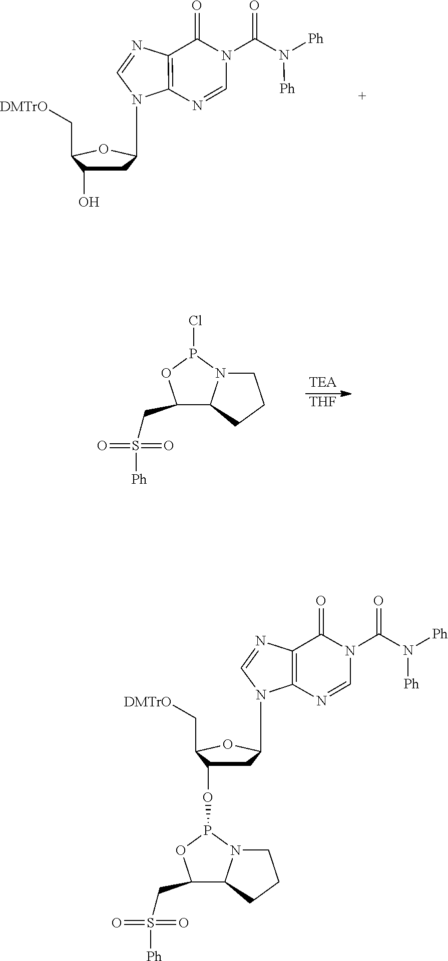

To a solution of dry 9-[(2R, 4R, 5R)-5-[[bis(4-methoxyphenyl)-phenyl-methoxy]methyl]-4-hydroxy-tetrahydrofuran-2-yl]-6-oxo-N, N-diphenyl-purine-1-carboxamide (10.0 g, 13.34 mmol) in THF (75 mL) was added triethylamine (4.65 mL, 33.34 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75M in THF, 32.01 mL, 24.01 mmol) was added dropwise. The ice/water bath was removed. The resulting slurry was stirred at rt for 3.5 hr. TLC and LCMS showed the reaction was complete. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a white foam (9.91 g, 71.9% yield). ¹H NMR (600 MHz, Chloroform-d) δ 8.07 (s, 1H), 7.92-7.87 (m, 2H), 7.79 (s, 1H), 7.57 (t, J=7.5 Hz, 1H), 7.47 (t, J=7.8 Hz, 2H), 7.46-7.34 (m, 6H), 7.34-7.26 (m, 5H), 7.25-7.04 (m, 8H), 6.83-6.77 (m, 4H), 6.25 (t, J=7.3 Hz, 1H), 5.06 (s, 1H), 4.98-4.83 (m, 1H), 4.17-4.05 (m, 1H), 3.777 (s, 3H), 3.776 (s, 3H), 3.65 (dq, J=10.0, 5.9 Hz, 1H), 3.55-3.45 (m, 1H), 3.48 (dd, J=14.6, 7.0 Hz, 1H), 3.37 (dd, J=14.6, 5.3 Hz, 1H), 3.33-3.22 (m, 2H), 3.14-3.09 (m, 1H), 2.71-2.59 (m, 1H), 2.51 (s, 1H), 1.93-1.88 (m, 1H), 1.83-1.75 (m, 1H), 1.70-1.62 (m, 1H), 1.17-1.07 (m, 1H); ³¹P NMR (243 MHz, Chloroform-d) δ 152.46 (major), 152.33 (minor); major:minor=56: 44; MS (ESI), 1033.48 [M+H]³⁰ .

### Synthesis of 1-(9-((2R, 4S, 5R)-5-((bis(4-methoxyphenyl)(phenyl)methoxy)methyl)-4-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydrofuran-2-yl)-8-oxo-8, 9-dihydro-7H-purin-6-yl)-3-(naphthalen-2-yl)urea

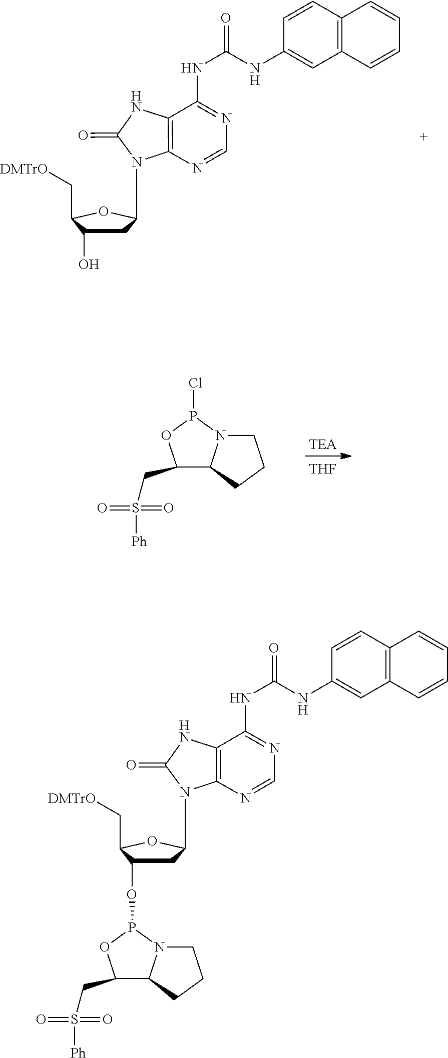

To a solution of dry 1-[9-[(2R, 4R, 5R)-5-[[bis(4-methoxyphenyl)-phenyl-methoxy]methyl]-4-hydroxy-tetrahydrofuran-2-yl]-8-oxo-7H-purin-6-yl]-3-(2-naphthyl)urea (3.0 g, 4.06 mmol) in THF (22.5 mL) was added triethylamine (1.41 mL, 10.15 mmol). The reaction flask was set in an ice/water bath. (3S, 3aS)-3-(benzenesulfonylmethyl)-1-chloro-3a, 4, 5, 6-tetrahydro-3H-pyrrolo[1, 2-c][1, 3, 2]oxazaphosphole (0.75M in THF, 9.75 mL, 7.31 mmol) was added dropwise. The ice/water bath was removed. The resulting slurry was stirred at rt for 3 hr. The mixture was filtered through celite, and the filtrate was concentrated. The resulting crude product was purified by normal phase column chromatography applying 0-100% EtOAc in acetonitrile (EtOAc contained 2.5% TEA, acetonitrile contained 1.0% TEA) as the gradient to afford the title compound as a brownish off-white foam (2.17 g, 52.3% yield). ¹H NMR (600 MHz, Chloroform-d) δ 11.97 (s, 1H), 10.46 (s, 1H), 9.69 (s, 1H), 8.24-8.11 (m, 2H), 8.04-7.96 (m, 1H), 7.96-7.89 (m, 2H), 7.85 (d, J=7.9 Hz, 1H), 7.74-7.67 (m, 1H), 7.61-7.57 (m, 1H), 7.51 (t, J=7.8 Hz, 3H), 7.46-7.40 (m, 3H), 7.39-7.34 (m, 1H), 7.31 (dt, J=8.9, 2.2 Hz, 4H), 7.24-7.18 (m, 2H), 7.14 (t, J=7.2 Hz, 1H), 6.79-6.70 (m, 4H), 6.34 (t, J=6.8 Hz, 1H), 5.20-5.07 (m, 2H), 4.07 (q, J=4.8 Hz, 1H), 3.78-3.64 (m, 7H), 3.49 (dt, J=14.9, 7.5 Hz, 2H), 3.45-3.38 (m, 2H), 3.35 (q, J=7.0 Hz, 1H), 3.28 (dd, J=10.2, 5.5 Hz, 1H), 3.06 (qd, J=10.0, 3.9 Hz, 1H), 2.32 (dq, J=13.0, 7.7, 6.4 Hz, 1H), 1.89 (tq, J=10.6, 4.5, 3.3 Hz, 1H), 1.84-1.74 (m, 1H), 1.73-1.65 (m, 1H), 1.19-1.10 (m, 1H); ³¹P NMR (243 MHz, Chloroform-d) δ 149.69 (major), 145.64 (minor); major:minor=83:17; MS (ESI), 1020.11 [M−H]⁻.

### Synthesis of N-(1-((2R, 5S, 6R)-6-((bis(4-methoxyphenyl)(phenyl)methoxy)methyl)-5-hydroxytetrahydro-2H-pyran-2-yl)-2-oxo-1, 2-dihydropyrimidin-4-yl)acetamide (WV-NU-223)

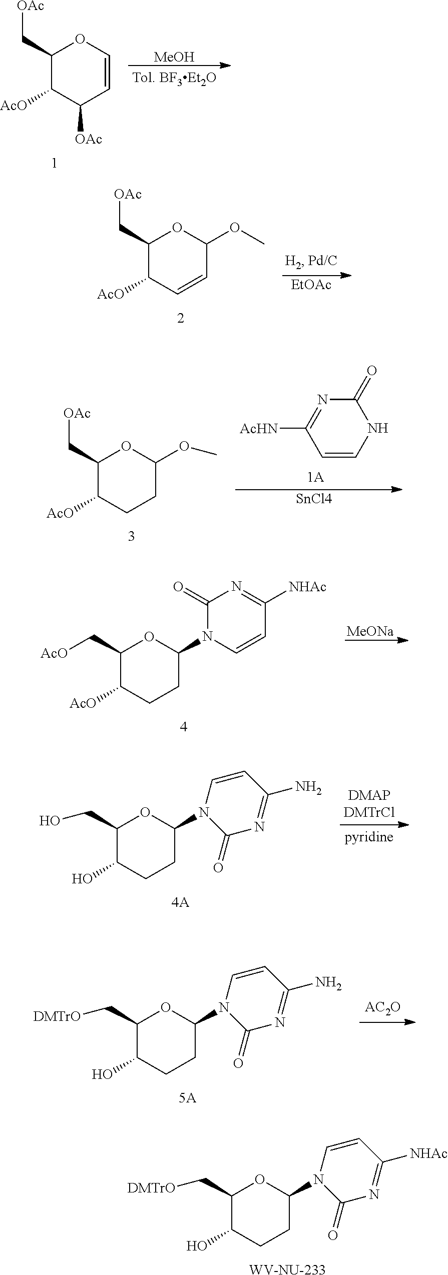

Step 1. Boron trifluoride diethyl etherate (22.16 g, 156.11 mmol) was added to a solution of Compound 1 (50 g, 183.65 mmol) in MeOH (12.95 g, 404.04 mmol, 16.35 mL, 2.2 eq.) and Tol. (600 mL) at 0° C. The mixture was stirred at 20° C. for 6.5 hrs. TLC indicated compound 1 was consumed completely and two new spots formed. The reaction was clean according to TLC. The solution was cooled to 0° C., and quenched with Et₃N (20.45 mL), after maintaining for 10 min at 0° C., Na₂CO₃ (19.45 g) was added to the solution. The filtrate was concentrated to give a residue, which was purified by silica gel column chromatography (SiO₂, Petroleum ether/Ethyl acetate=100/1 to 1/1, with 1.0 vol % Et₃N). Compound 2 (44 g, 98.09% yield) was obtained as a colorless oil. ¹H NMR (CHLOROFORM-d, 400 MHz): δ=5.73-5.87 (m, 2H), 5.25 (dd, J=9.7, 1.5 Hz, 1H), 4.86 (s, 1H), 4.13-4.25 (m, 2H), 3.93-4.03 (m, 1H), 3.39 (s, 3H), 2.04 (s, 3H), 2.02 ppm (s, 4H). TLC (Petroleum ether:Ethyl acetate=5:1) Rf=0.23.

Step 2. To a solution of compound 2 (44 g, 180.15 mmol) in EtOAc (400 mL) was added Pd/C (0.07 mg, 180.15 mmol, 10% purity). The mixture was stirred at 25° C. for 18 hrs under H₂(15 Psi) atmosphere. TLC indicated compound 2 was consumed completely and one new spot formed. The reaction was clean according to TLC. The insoluble material was removed by filtration through a pad of celite. The pad was washed with EtOAc (100 mL). The filtrate was concentrated. The residue was purified by column chromatography (SiO₂, Petroleum ether/Ethyl acetate=100/1 to 0/1). Compound 3 (43 g, 96.93% yield) was obtained as a colorless oil. ¹H NMR (CHLOROFORM-d, 400 MHz): δ=4.67-4.80 (m, 2H), 4.22-4.31 (m, 1H), 4.11 (dd, J=11.9, 2.3 Hz, 1H), 3.90 (ddd, J=10.0, 5.3, 2.3 Hz, 1H), 3.37 (s, 3H), 2.09 (s, 3H), 2.04-2.06 (m, 3H), 1.81-2.02 ppm (m, 4H). TLC: (Petroleum ether:Ethyl acetate=5:1) Rf=0.30.

Step 3. To a solution of compound 1A (37.44 g, 244.46 mmol) in MeCN (500 mL) was added BSA (71.04 g, 349.23 mmol, 86.32 mL) and compound 3 (43 g, 174.61 mmol) and SnCl₄ (68.24 g, 261.92 mmol, 30.60 mL). The mixture was stirred at 45° C. for 2 hr. TLC indicated compound 1A was consumed completely and two new spot formed. The reaction was clean according to TLC. The reaction mixture was partitioned between H₂O (200 mL) and EtOAc (500 mL). The organic phase was separated, washed with brine 30 mL, dried over Na₂SO₄, filtered and concentrated under reduced pressure to give a residue. The residue was purified by column chromatography (SiO₂, Petroleum ether/Ethyl acetate=100/1 to 0/1). Compound 4 (34 g, crude) was obtained as a yellow oil.

Step 4. To a solution of compound 4 (24 g, 65.33 mmol) in MeOH (5 mL) was added CH₃ONa (10.59 g, 196.00 mmol). The mixture was stirred at 15° C. for 12 hr. HPLC showed compound 4 was consumed completely and one main peak with desired mass was detected. The filter liquor was concentrated in vacuo. The crude product was purified by reverse-phase HPLC (0.1% FA condition). Compound 4A (13 g, 82.48% yield) was obtained as a white solid.

Step 5. To a solution of compound 4A (13 g, 53.89 mmol) in pyridine (200 mL) was added DMT-C1 (18.26 g, 53.89 mmol). The mixture was stirred at 25° C. for 6 hr. LCMS showed compound 4A was consumed completely and one main peak with desired m/z. The reaction mixture was partitioned between H₂O (500 mL) and EtOAc (1000 mL). The organic phase was separated, washed with brine (50 mL), dried over Na₂SO₄, filtered and concentrated under reduced pressure to give a residue. The residue was purified by column chromatography (SiO₂, Dichloromethane: Methanol=100/1 to 0/1). Compound 5A (13 g, 44.38% yield) was obtained as a white solid. LCMS: (M−H⁺)=542. TLC (Dichloromethane: Methanol=10:1), Rf=0.33.

Step 6. To a solution of compound 5A (13 g, 23.91 mmol) in Pyr. (130 mL) was added Ac₂O (2.44 g, 23.91 mmol, 2.24 mL). The mixture was stirred at 25° C. for 12 hr. TLC (Petroleum ether:Ethyl acetate=0:1) indicated compound 5A was consumed completely and one new spot formed. The reaction was clean according to TLC. The mixture was concentrated in vacuum. The residue was purified by column chromatography (SiO₂, Petroleum ether/Ethyl acetate=100/1 to 0/1). Compound WV-NU-223 (7.7 g, 52.78% yield, 96% purity) was obtained as a white solid. ¹H NMR (DMSO-d₆, 400 MHz): δ=10.93 (s, 1H), 8.12 (d, J=7.5 Hz, 1H), 7.40 (br d, J=7.4 Hz, 2H), 7.14-7.34 (m, 8H), 6.79-6.91 (m, 4H), 5.73 (br d, J=10.4 Hz, 1H), 4.89 (d, J=6.1 Hz, 1H), 3.73 (s, 6H), 3.59-3.66 (m, 1H), 3.26 (br d, J=9.4 Hz, 1H), 3.15 (br dd, J=10.0, 6.7 Hz, 1H), 2.12 (s, 3H), 1.93 (br d, J=10.2 Hz, 2H), 1.53-1.72 ppm (m, 2H). LCMS=(M−H⁺)=585.6. TLC (Petroleum ether:Ethyl acetate=0:1), Rf=0.43.

### Synthesis of N-(1-((2R, 5S, 6R)-6-((bis(4-methoxyphenyl)(phenyl)methoxy)methyl)-5-(((1S, 3S, 3aS)-3-((phenylsulfonyl)methyl)tetrahydro-1H, 3H-pyrrolo[1, 2-c][1, 3, 2]oxazaphosphol-1-yl)oxy)tetrahydro-2H-pyran-2-yl)-2-oxo-1, 2-dihydropyrimidin-4-yl)acetamide (CSM-019-L-PSM)

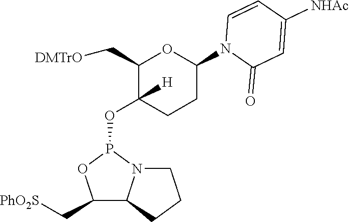

Amidite 6 was synthesized using general procedure. Yield, 74%. ³¹P NMR (243 MHz, CDCl₃) δ 149.44; MS (ES) m/z calculated for C₄₅H₄₉N₄O₁₀PS [M+K]⁺ 907.25, Observed: 907.14 [M+K]⁺.

### Synthesis of 3-((2R,3R,4S,5R)-5-((bis(4-methoxyphenyl)(phenyl)methoxy)methyl)-4-hydroxy-3-(trifluoromethyl)tetrahydrofuran-2-yl)-3H-benzo[b]pyrimido[4,5-e][1,4]oxazin-2(1OH)-one (WV-NU-243)

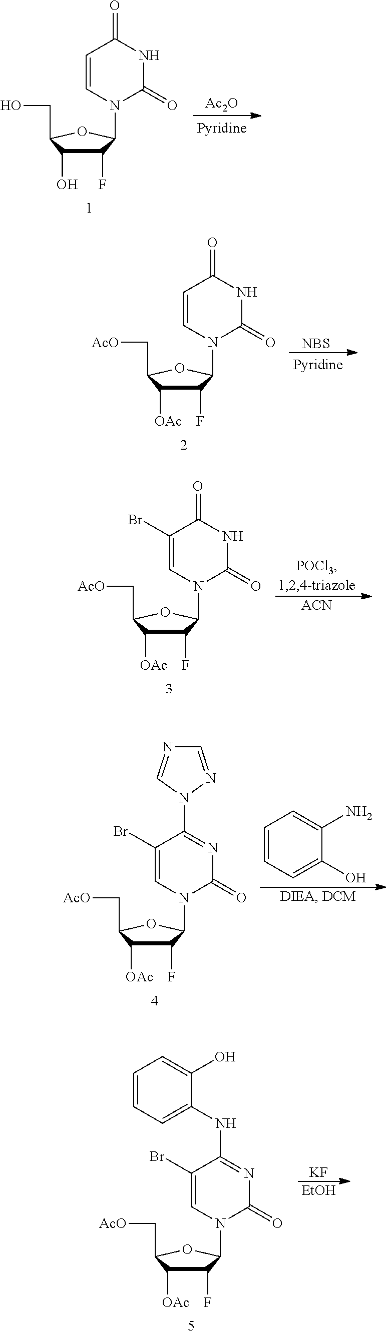

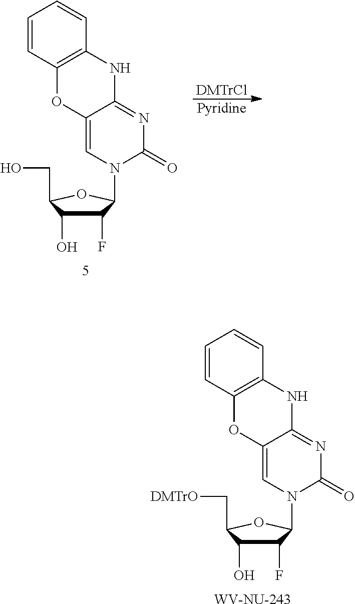

Step 1. For two batches: to a solution of compound 1 (100 g, 406.19 mmol.) in pyridine (1000 mL) was added Ac₂O (165.87 g, 1.62 mol.). The mixture was stirred at 20° C. for 18 hr. TLC indicated compound 1 was consumed completely and one new spot formed. The reaction mixture of two batches were combined and was concentrated under reduced pressure to remove solvent. The residue was washed with petroleum ether 1000 mL *2. Compound 2 (268 g, 99.89% yield) was obtained as a white solid. ¹H NMR (400 MHz, DMSO-d6) δ=11.47 (br s, 1H), 7.71 (d, J=8.0 Hz, 1H), 5.93-5.82 (m, 1H), 5.68 (d, J=8.0 Hz, 1H), 5.62-5.44 (m, 1H), 5.26 (ddd, J=5.4, 7.9, 17.3 Hz, 1H), 4.39-4.31 (m, 1H), 4.30-4.24 (m, 1H), 4.21-4.12 (m, 1H), 2.11 (s, 3H), 2.04 (s, 3H). TLC:Petroleum ether:Ethyl acetate=0:1, Rf=0.8.

Step 2. For two batches: To a solution of compound 2 (134 g, 405.73 mmol) in Py (1300 mL) was added NBS (93.88 g, 527.45 mmol). The mixture was stirred at 20° C. for 18 hr. TLC indicated compound 2 was consumed completely and one new spot formed. The reaction mixture of two batches were concentrated under reduced pressure to remove solvent. The residue was purified by column chromatography (SiO₂, Petroleum ether/Ethyl acetate=1/1 to 0/1). Compound 3 (323 g, 97.28% yield) was obtained as a yellow solid. ¹H NMR (400 MHz, DMSO-d6) δ=11.99 (s, 1H), 8.20 (s, 1H), 5.97-5.79 (m, 1H), 5.63-5.43 (m, 1H), 5.27 (ddd, J=5.3, 7.9, 18.0 Hz, 1H), 4.40-4.27 (m, 2H). 4.25-4.17 (m, 1H), 2.09 (d, J=17.6 Hz, 6H). LCMS (M−H+): 411.0. TLC: Petroleum ether:Ethyl acetate=1:2, Rf=0.8.

Step 3. For two batches: A mixture of 1H-1, 2, 4-TRIAZOLE (81.02 g, 1.17 mol.), POCl₃ (44.97 g, 293.28 mmol) in ACN (1300 mL) was added TEA (148.39 g, 1.47 mol) slowly at 0° C., 30 min later, a solution of compound 3 (30 g, 73.32 mmol) in ACN (200 mL) was added, the mixture was stirred at 15° C. for 1 hr. TLC indicated compound 3 was consumed completely and one new spot formed. Excess TEA (150 mL) and water (50 mL) were added and stirred for 30 min. The solvent was evaporated to a residue and partitioned between dichloromethane (500 mL) and sodium bicarbonate solution (200 mL). The mixture of two batches were combined and aqueous layer was extracted with dichloromethane (500 mL*3) and the combined organic layer was washed with brine 500 mL, dried over sodium sulfate. The solvent was evaporated to give a light yellow solid which was dried under high vacuum. Compound 4 (60 g, crude) was obtained as a yellow solid. TLC:Petroleum ether:Ethyl acetate=1:2, Rf=0.4.

Step 4. For two batches: To a solution of compound 4 (60 g, 130.37 mmol.) in DCM (1200 mL) was added DIEA (42.12 g, 325.94 mmol) and 2-aminophenol (35.57 g, 325.94 mmol.). The mixture was stirred at 20° C. for 12 hr. LCMS showed the desired mass was detected. The reaction mixture was quenched by addition 5% citric acid (500 ml) and H2O 500 mL, then mixture of two batches were combined and extracted with dichloromethane (1000 ml*2) and the combined organic layer was dried over sodium sulfate and evaporated to give a crude. The crude was washed with ethyl acetate 1000 mL*3. Compound 5 (43 g, 65.93% yield) was obtained as a brown solid. LCMS (M−H+): 499.2.

Step 5. To a solution of compound 5 (30 g, 59.97 mmol) in EtOH (1.5 L) was added KF (69.68 g, 1.20 mol). The mixture was stirred at 95° C. for 12 hr. LCMS showed the desired mass was detected. The reaction mixture was cooled to 15° C. then filtered and concentrated under reduced pressure to give a residue. Compound 6 (20 g, crude) was obtained as a brown solid. LCMS (M−H+): 336.0.

Step 6. To a solution of compound 6 (20 g, 59.65 mmol) in Py (1000 mL) was added DMTCl (22.23 g, 65.62 mmol). The mixture was stirred at 80° C. for 10 hr. LCMS showed the desired mass was detected. The reaction mixture was quenched by addition methanol 50 mL at 15° C., and then concentrated under reduced pressure to give a residue. The residue was purified by column chromatography (SiO₂, Petroleum ether/Ethyl acetate=1/0 to 0/1, then Ethyl acetate: Methanol=1/0 to 0/1). Then the crude product was purified by prep-HPLC (column: Agela DuraShell C18 250*70 mm*10 um; mobile phase: [water (NH₄HCO₃)-ACN];B %: 42%-72%, 18 min). Compound WV-NU-243 (3.68 g, 67% yield) was obtained as a yellow solid. ¹H NMR (400 MHz, DMSO-d₆) δ=10.77-9.94 (m, 1H), 7.86-7.58 (m, 1H), 7.34-7.25 (m, 2H), 7.24-7.16 (m, 1H), 7.11 (br d, J=7.6 Hz, 2H), 7.01 (d, J=8.8 Hz, 4H), 6.86 (d, J=8.9 Hz, 4H), 6.73 (d, J=8.6 Hz, 1H), 6.68 (s, 1H), 6.56 (dd, J=2.1, 8.6 Hz, 1H), 5.86 (dd, J=1.2, 17.1 Hz, 1H), 5.58 (br d, J=6.3 Hz, 1H), 5.33 (brt, J=4.4 Hz, 1H), 5.04-4.78 (m, 1H), 4.24-4.09 (m, 1H), 3.88 (br d, J=7.6 Hz, 1H), 3.78 (br s, 1H), 3.73 (s, 6H), 3.66-3.57 (m, 1H). LCMS (M−H+): 636.2, purity: 100%. TLC (Petroleum ether: Ethyl acetate=1:1, Rf=0.3).

### Synthesis of 3-((2R,3R,4R,5R)-5-((bis(4-methoxyphenyl)(phenyl)methoxy)methyl)-3-fluoro-4-(((1 S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-3H-benzo[b]pyrimido[4,5-e][1,4]oxazin-2(1OH)-one (L-PSM-WV-NU-243 Amidite)

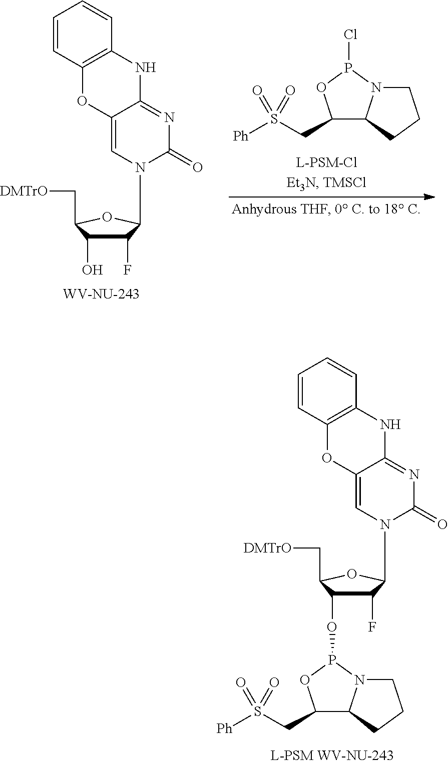

Nucleosides WV-NU-243 (1.72 g, 2.70 mmol, 1.0 eq.) in a 250 mL size three necked flask was azeotroped with anhydrous toluene (30 mL) and was dried for 48 hrs on high vacuum. To the flask was added anhydrous THF (10 mL) under argon and solution was cooled to −10° C. To the reaction mixture was added triethyl amine (4.0 eq.) followed by addition of TMSCl (1.0 eq) and then L-PSM-C1 (0.8 M) solution (2.0 eq.) over the period of 10 min. The reaction mixture was warmed to room temperature and reaction progress was monitored by HPLC. After disappearance of starting material, reaction was quenched by addition of water and dried by addition of molecular sieve. The reaction mixture was filtered through fritted glass tube. Reaction flask and precipitate was washed with anhydrous THF (25 mL). Obtained filtrate was collected and solvent was removed under reduced pressure. The residue was purified by column chromatography (SiO2, 40-100% Ethyl acetate in Hexanes) to give L-PSM-WV-NU-243 Amidite off yellow solid (2.1 g, 84% yield). LCMS: C4₅H₄₆N₅O₉PS (M−H⁺): 919.27. ¹H NMR (600 MHz, CDCl₃) δ 10.95 (s, 1H), 7.90-7.84 (m, 2H), 7.63 (t, J=6.6 Hz, 1H), 7.61-7.57 (m, 1H), 7.52 (s, 1H), 7.49 (t, J=7.9 Hz, 2H). 7.47-7.42 (m, 2H), 7.38-7.32 (m, 4H), 7.29 (t, J=7.7 Hz, 2H), 7.22-7.16 (m, 1H), 6.88-6.78 (m, 5H), 6.74 (td, J=7.8, 1.7 Hz, 1H), 6.28 (dt, J=8.1, 1.4 Hz, 1H), 6.00 (d, J=16.0 Hz, 1H), 5.10 (d, J=4.1 Hz, 2H), 5.00 (dt, J=12.3, 5.4 Hz, 2H), 4.80-4.65 (m, 1H), 4.14 (d, J=8.1 Hz, 1H), 3.70 (d, J=11.8 Hz, 8H), 3.56-3.40 (m, 4H), 3.36 (dd, J=14.6, 5.9 Hz, 1H), 3.14 (qd, J=10.0, 4.1 Hz, 1H), 1.85 (dtt, J=11.8, 7.7, 3.3 Hz, 1H), 1.76 (dt, J=12.0, 8.5 Hz, 1H), 1.66 (dtd, J=12.3, 6.0, 2.8 Hz, 1H), 1.16-1.04 (m, 1H). ³¹P NMR (243 MHz, CDCl₃) 6=155.40. ⁹F NMR (565 MHz, CDCl₃) 6=200.12. ³C NMR (151 MHz, CDCl₃) δ 171.81, 159.27, 159.24, 156.20, 154.29, 144.82, 142.89, 139.74, 136.27, 135.97, 134.71, 130.89, 130.71, 129.99, 128.92, 128.90, 128.82, 128.67, 127.66, 126.66, 124.90, 124.59, 121.99, 118.74, 115.89, 113.95, 113.93, 94.24, 92.97, 89.32, 89.10, 87.60, 81.76, 81.74, 75.38, 75.32, 69.43, 69.36, 69.33, 69.26, 66.78, 66.75, 61.06, 60.92, 58.77, 58.74, 55.84, 55.82, 47.18, 46.95, 28.03, 26.69, 26.67, 21.72, 14.87.

### Synthesis of 3-((2R,3R,4R,5R)-5-((bis(4-methoxyphenyl)(phenyl)methoxy)methyl)-4-hydroxy-3-methoxytetrahydrofuran-2-yl)-3H-benzol[b]pyrimido[4,5-e][1,4]oxazin-2(10H)-one (WV-NU-253)

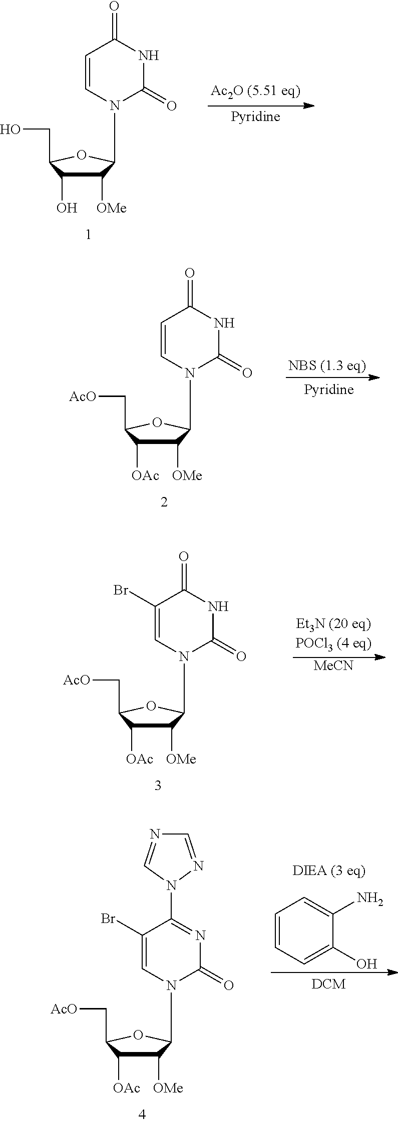

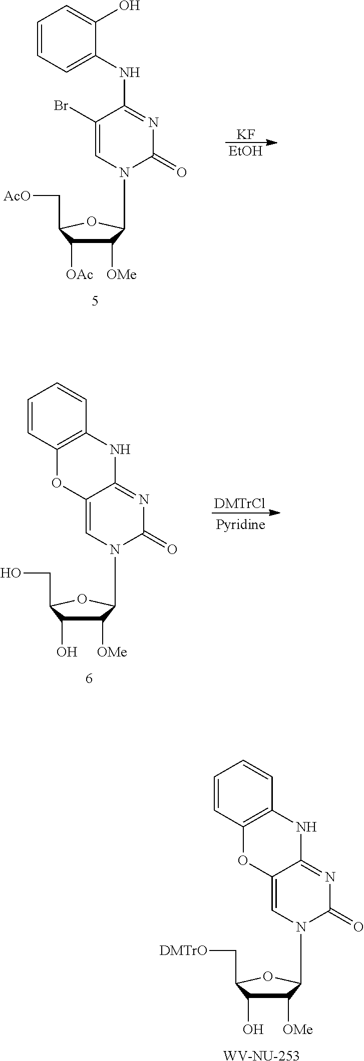

Step 1. To a solution of compound 1 (90 g, 348.53 mmol) in Pyridine (720 mL) was added Ac₂O (196.20 g, 1.92 mol) and the reaction mixture was stirred at 25° C. for 18 h. LCMS showed the product was detected. The residue was dissolved in dichloromethane (1000 ml) and washed with saturated sodium bicarbonate solution (4*1000 ml). Aqueous layer was extracted with dichloromethane (2*750 ml) and the combined organic layers was dried over sodium sulfate and evaporated. Without any further purification used for next step. Compound 2 (100 g, 83.82% yield) was obtained as yellow oil. ¹H NMR (400 MHz, DMSO-d6) δ=11.60-11.33 (m, 1H), 8.71-8.49 (m, 1H), 7.82-7.74 (m, 1H), 7.70 (d, J=8.1 Hz, 1H), 7.42-7.35 (m, 1H), 5.83 (d, J=5.7 Hz, 1H), 5.74 (d, J=8.1 Hz, 1H), 5.20 (br s, 1H), 4.27-4.18 (m, 4H), 3.29 (s, 3H), 2.10 (s, 3H), 2.07 (s, 3H). LCMS (M+H+): 341.2.

Step 2. To a solution of compound 2 (100 g, 292.14 mmol) in Pyridine (1000 mL) was added NBS (67.59 g, 379.78 mmol). The reaction mixture was stirred at 25° C. for 18h. LCMS showed the product was detected. The reaction mixture was diluted with dichloromethane (1000 ml) and washed with saturated sodium bicarbonate solution (3×800 ml). Aqueous layer was extracted with dichloromethane (2×500 ml) and the combined organic layer was dried over sodium sulfate and evaporated. The residue was purified by column chromatography (SiO₂, Petroleum ether/Ethyl acetate=10/1 to 0/1). Compound 3 (117 g, 95.08% yield) was obtained as a brown solid. 1H NMR (400 MHz, DMSO-d6) δ=11.97 (s, 1H), 8.13 (s, 1H), 5.80 (d, J=4.8 Hz, 1H), 5.19 (t, J=5.2 Hz, 1H), 4.31-4.19 (m, 4H), 3.33-3.31 (m, 3H), 2.10 (d, J=2.6 Hz, 6H). LCMS (M+H⁺): 444.3. TLC (Petroleum ether:Ethyl acetate=0:1), Rf=0.37.

Step 3. To a suspension of 1H-1, 2, 4-triazole (91.83 g, 1.33 mol) and POCl₃ (50.96 g, 332.39 mmol, 30.89 mL) in ACN (1000 mL) was added Et₃N (168.17 g, 1.66 mol, 231.32 mL) slowly at 0° C. (ice bath). After stirring for 20 min a solution of compound 3 (35 g, 83.10 mmol) in ACN (500 mL) was added to the reaction flask. Ice bath was removed and the mixture was stirred at 25° C. for 3 h. TLC showed the product was detected. Excess triethylamine (700 mL) and water (300 mL) were added and stirred for 30 min. The solvent was evaporated to a residue and partitioned between dichloromethane (500 * 3 mL) and sodium bicarbonate solution (200 mL). Aqueous layer was extracted with dichloromethane and the combined organic layer was dried over sodium sulfate. The solvent was evaporated to give a light yellow solid which was dried under high vacuum. The mixture was without further purification used for next step. Compound 4 (39 g, crude) was obtained as yellow oil. TLC (Petroleum ether:Ethyl acetate=1:2), Rf=0.36.

Step 4. To a solution of compound 4 (39 g, 82.58 mmol) in DCM (500 mL) was added 2-aminophenol (27.04 g, 247.75 mmol) and DTEA (32.02 g, 247.75 mmol, 43.15 mL) The reaction mixture was stirred at 25° C. for 2h. TLC showed the product was detected. The reaction mixture was quenched by addition 5% citric acid (200 ml). Aqueous layer was extracted with dichloromethane (200 ml) and the combined organic layer was dried over sodium sulfate and evaporated to give a foam. The crude product was triturated with (Petroleum ether:Ethyl acetate=1:1) 500 ml at 25° C. for 12h. Compound 5 (20 g, crude) was obtained as a brown solid. TLC (Petroleum ether:Ethyl acetate=1:2), Rf=0.57.

Step 5. To a solution of compound 5 (30 g, 58.56 mmol), KF (34.02 g, 585.59 mmol) in EtOH (1500 mL), and the mixture was stirred at 90° C. for 72 hr. LCMS showed the compound 5 was consumed and the desired mass was found. The mixture was concentrated to get the crude and the mixture was washed with DCM:MeOH=10:1 (2000 mL), filtered and the filtrated was concentrated to get the compound 6 (20 g, 98.33% yield) as a yellow oil. LCMS: (M+H+): 348.1.

Step 6. To a solution of compound 6 (20 g, 57.58 mmol) in PYRIDINE (2000 mL) was added DMTCl (25.36 g, 74.86 mmol) and the mixture was stirred at 15° C. for 12 hr. LCMS showed the compound 6 was consumed and the desired mass was obtained. The mixture was concentrated to get the crude. The residue was purified by MPLC (DCM:MeOH=20:1, 10:1). The crude product was triturated with DCM (10 mL): MTBE (10 mL) was added to the stirred Petroleum ether (10 mL) at 15° C. for 5 min. Filtered and the cake was dried to get the WV-NU-253 (10.7 g, 26.94% yield) as a yellow solid. ¹HNMR (400 MHz, CHLOROFORM-d) δ=10.01 (br s, 1H), 7.52 (s, 1H), 7.41 (d, J=7.5 Hz, 2H), 7.35-7.28 (m, 5H), 7.24 (t, J=7.6 Hz, 2H), 7.17-7.12 (m, 1H), 6.82-6.70 (m, 6H), 6.29 (dd, J=1.3, 7.8 Hz, 1H), 5.88 (s, 1H), 4.44-4.37 (m, 1H), 3.94 (br d, J=8.0 Hz, 1H), 3.81 (d, J=5.1 Hz, 1H), 3.71-3.62 (m, 9H), 3.51-3.39 (m, 2H), 2.55 (br d, J=9.3 Hz, 1H). LCMS: (M−H+):648.3, LCMS purity: 94.193%. TLC:DCM:MeOH=10:1, Rf=0.38.

### Synthesis of 3-((2R, 4S, 5R)-5-((bis(4-methoxyphenyl)(phenyl)methoxy)methyl)-4-hydroxytetra hydrofuran-2-yl)-benzo[b]pyrimido[4, 5-e][1, 4]oxazin-2(10H)-one (WV-NU-242)

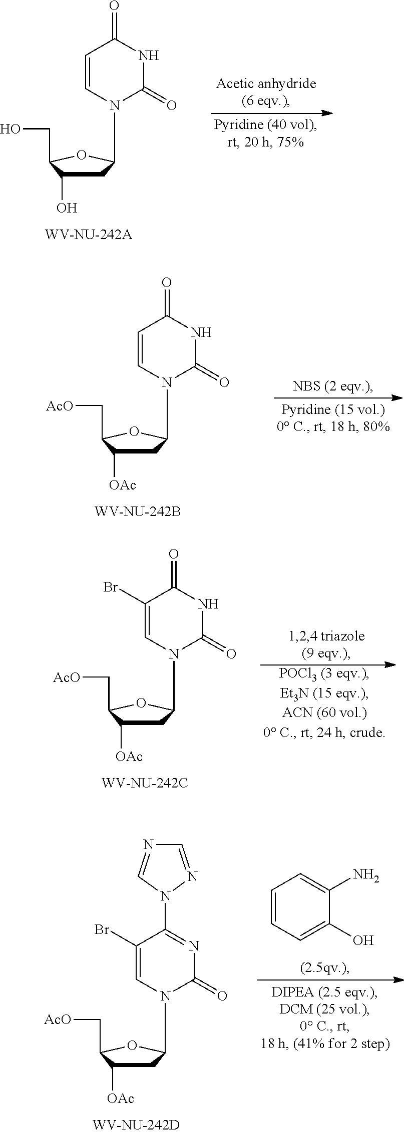

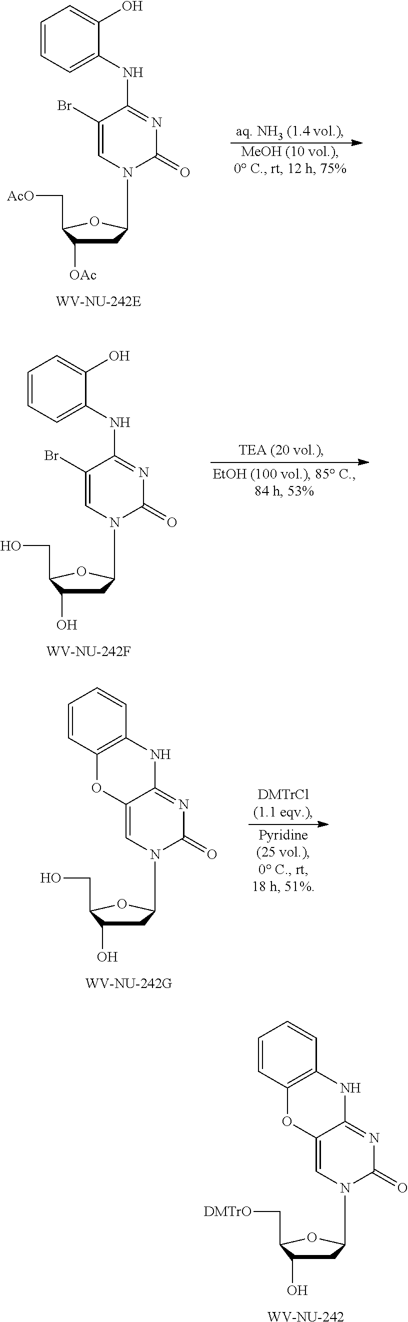

Step 1. To a stirred solution of (WV-NU-242A) (50 g, 0.219 mol) in dry pyridine (750 mL, 15 vol.) was added acetic anhydride (124.3 mL, 1.315 mol.) drop-wise over a period of 30 min at 0° C. Then the reaction was further stirred at rt for 18 h. Progress of the reaction was monitored by TLC. The reaction mixture was concentrated under vacuum to get a crude mass. The mass was co-distilled with toluene (2×100 mL), and dried under vacuum to afford an off white solid (51 g). TLC Mobile phase details: 7% MeOH in DCM. ¹H NMR (500 MHz, DMSO-d₆): δ in ppm=10.39 (s, 1H), 7.66 (d, 1H, J1=8.3 Hz), 7.20 (m, 1H), 6.15 (dd, 1H, J1=8.3 Hz, , J2=6.2 Hz ), 5.70 (d, 1H, J1=8.3 Hz) 5.18 (m, 1H), ) 5.18 (m, 1H) ), 4.22 (m, 2H) ) 4.17 (m, 1H) ) 2.47 (m, 1H), 2.31 (m, 2H) 2.06 (m, 6H, J1=6.9 Hz) MS: m czalcd for C₁₃H₁₆N₂O₇ 312.3; found [M+H⁺]⁺, 313.14.

Step 2. To a stirred solution of (WV-NU-242B) (40 g, 0.128 mol) in dry pyridine (600 mL, 15 vol.) was added freshly crystallized NBS (41.07 g, 0.2305 mol.) portion-wise over a period of 30 min at 0° C. Above reaction was stirred at rt for 18 h. Progress of the reaction was monitored by TLC. Then reaction was concentrated under vacuum. The crude was dissolved in DCM (600 mL), washed with sat.NaHCO₃ (2×100 mL), dried over Na₂SO₄ and concentrated under vacuum to get gummy mass. The crude was purified by column chromatography over silica gel (230-400 mesh) eluted in 30% EtOAc/Hexane to get as an off white solid (WV-NU-243C) (38 g, 76%). TLC Mobile phase details: 60% EtOAc in Hexane. ¹H NMR (400 MHz, DMSO-d₆): δ in ppm=11.09 (s, 1H), 8.06 (s, 1H), 6.12 (dd, 1H, J1=87.9 Hz, , J2=6.3 Hz ), 5.18 (m, 1H), 4.26 (d, 2H, J1=4.7 Hz ) 4.19 (m, 1H), ) 2.57 (s, 1H) ), 2.53 (d, 1H, J1=7.6 Hz) ) 2.31 (ddd, 1H) ) 2.08 (s, 3H), 2.06 (s, 3H), MS: m/z calcd for C₁₃H₁₅BrN₂O₇, 391.2; found [M+2]⁺, 393.

Step 3. To a stirred solution of 1, 2, 4-triazole (63.52 g, 0.92 mol) in dry acetonitrile (1.7 Lit, 50 vol.), at 0° C. was added POCl₃ (28.63 mL, 0.3063 mol), followed by trimethylamine (209 mL, 1.534 mol) dropwise over a period of 40 min. Then the mixture was stirred at 0° to 10° C. for 2 h. To this mixture was added a solution of (WV-NU-242C) (40 g, 0.1023 mol) in dry acetonitrile (300 mL) dropwise over a period of 1 h at OoC. Then the mixture was allowed to rt and stirred for 24 h. Progress of the reaction was monitored by TLC. The reaction mixture was concentrated under vacuum. The crude was dissolved in DCM (1 Lit), washed with sat.NaHCO₃ (200 mL×2), dried over Na₂SO₄ and concentrated under vacuum to afford a yellowish solid (WV-NU-242D) (40 g), TLC Mobile phase details: 60% EtOAc in Hexane. ¹H NMR (500 MHz, DMSO-d₆): δ in ppm=9.28 (s, 1H), 8.66 (s, 1H), 8.41 (s, 1H), 6.11 (t, 1H, 1H, J1=6.5), 5.24 (m, 1H), 4.4 (m, 1H), 4.33 (ddd, 2H, J1=21.0 J2=12.1 J3=4.3), 2.59 (m, 2H, ), 2.09 (d, 6H, J1=4.8), MS: m/z calcd for C₁₅H₁₆BrN₂O₆, 442.2; found 444.08. [M+2]⁺.

Step 4. To a stirred solution of (WV-NU-242D) (40 g, 0.0907 mol) in dry acetonitrile (800 mL, 20 vol.), was added 2-Aminophenal (24.71 g, 0.2267 mol.), DBU (33.79 ml, 0.2267 mol), dropwise over a period of 20 min at 0° C. Above reaction was stirred at rt for 18 h. Progress of the reaction was monitored by TLC. Then reaction was concentrated under vacuum to get crude compound The crude was purified by column chromatography over silica gel (230-400 mesh) eluted in 50% EtOAc/Hexane to get a reddish solid (WV-NU-243C) (20 g, 41% for 2 step). TLC Mobile phase details: 70% EtOAc in Hexane. ¹H NMR (400 MHz, DMSO-d₆): δ in ppm=10.23 (s, 1H), 8.41 (s, 1H), 8.12 (dd, 1H, J1=8.2 Hz, , J2=1.5 Hz ), 8.10 (s, 1H), 7.02 (m, 1H), 6.93 (dd, 1H, J1=8.8 Hz, , J2=1.3 Hz ), 6.85 (m, 1H), 6.15 (m, 1H), 5.19 (dd, 1H, J1=6.1 Hz, J2=2.8 Hz) 4.26 (m, 3H), ) 2.38 (s, 2H) ), 2.08 (d, 1H, J1=7.7 Hz), MS: m/z calcd for C₁₉H₂₀BrN₃O₇, 482.3; found 484.05 [M+2]⁺.

Step 5. To a stirred solution of (WV-NU-242E) (20 g, 0.04149 mol) in methanol (200 mL, 10 vol.) was added 25% aq. ammonia solution (28 mL 1.4 vol) dropwise over a period of 10 min at 0° C. Above reaction was stirred at rt for 12 h. Progress of the reaction was monitored by TLC. Then reaction mixture was concentrated under vacuum; co-distil with toluene (2×100 ml), and dried to get a crude gummy mass. The crude mass was purified by column chromatography over silica gel (230-400 mesh) eluted in 5% MeOH/DCM to get a yellowish solid (WV-NU-243F) (12 g, 75%). TLC Mobile phase details: 10% MeOH in DCM. ¹H NMR (500 MHz, DMSO-d₆): δ in ppm=10.25 (s, 1H), 8.46 (s, 1H), 8.32 (s, 1H), 8.18 (dd, 1H, J1=7.6 Hz, , J2=1.4 Hz ), 7.01 (td, 1H, J1=7.2 Hz, , J2=1.6 Hz ), 6.93 (dd, 1H, , J1=8.3 Hz, , J2=1.4 Hz), 6.85 (m, 1H), 6.11 (t, 1H, J1=6.2 Hz, ), 5.25 (d, 1H, J1=4.1 Hz), 5.19 (t, 1H, J1=4.8 Hz), 4.24 (m, 1H, ) 3.82 (q, 1H, , J1=3.2 Hz), ) 3.63 (dd, 2H, J1=39.2 Hz, J2=7.7 Hz, J3=3.9 Hz,) ), 2.21 (qd, 1H, J1=6.5 Hz, J2=4.3 Hz), ), 2.08 (m, 1H, ) MS: m/z caled for C₁₅H₁₆BrN₃O₅ 398.3; found 400.05 [M+2]⁺.

Step 6. To a stirred solution of (WV-NU-242F) (7 g, 0.01799 mol) in anhydrous ethanol (350 mL, 50 vol.) was added triethylamine (140 mL, 20 vol) dropwise over a period of 10 min at rt. Above reaction was stirred at 85° C. for 80 h. Progress of the reaction was monitored by TLC. Then reaction was concentrated under vacuum to get a crude which was purified by column chromatography over silica gel (230-400 mesh) eluted in 7% MeOH/DCM to get as a yellowish solid (WV-NU-243G) (3 g, 52%). TLC Mobile phase details: 10% MeOH in DCM. ¹H NMR (400 MHz, DMSO-d₆): δ in ppm=7.67 (s, 1H), 6.94 (m, 2H), 6.81 (m, 2H), 6.12 (t, 1H, J1=6.7 Hz, ), 4.22 (m, 1H), 3.80 (m, 1H, ), 3.60 (m, 2H), 3.09 (m, 2H, ), 2.04 (m, 2H, ), 1.17 (t, 3H, J1=7.3 Hz) MS: m/z calcd for C₁₅H₁₅N₃O₅, 317.3; found 316.16 [M−1]⁺.

Step 7. To a stirred solution of (WV-NU-242G) (3 g, 0.0094 mol) in dry pyridine (75 mL, 25 vol.) was added DMTCl (3.51 g, 0.0104 mol) portion-wise over a period of 30 min at 0° C. Above reaction was stirred at for 18 h. Progress of the reaction was monitored by TLC. Then reaction was concentrated under vacuum to get a crude mass. The crude was dissolved in DCM (100 mL), washed with sat.NaHCO₃ (50 mL×2), dried over Na₂SO₄ and concentrated under vacuum to afford a yellow solid. The crude was purified by column chromatography over silica gel (230-400 mesh) eluted in 3% EtOH/DCM to get as a yellowish solid (WV-NU-243) (3 g, 51%). TLC Mobile phase details: 7% EtOH in DCM. ¹H NMR (400 MHz, DMSO-d₆): δ in ppm =7.41 (m, 2H), 7.31 (m, 7H), 7.22 (t, 1H, J1=6.7 Hz), 6.85 (m, 8H), 6.11 (t, 1H, J1=6.5 Hz), 5.27 (s, 1H, ), 4.28 (dd, 1H, J1=8.9 Hz, J2=5.0 Hz), 3.89 (m, 1H, ), 3.72 (m, 7H, ), 3.26 (q, 1 H, 11=5.2 Hz), 3.11 (dd, 1H, J1=10.4 Hz, J2=2.7 Hz), 2.162 (t, 1H, J1=6.0 Hz) MS: m/z caled for C₃₆H₃₃N₃O₇, 619.7; found 618.30 [M−1]⁺.

### Synthesis of 1-((2R,4S,5R)-5-((bis(4-methoxyphenyl)(phenyl)methoxy)methyl)-4-hydroxytetrahydrofuran-2-yl)-1,5-dihydro-4H-imidazo[4,5-d]pyridazin-4-one (WV-NU-265)

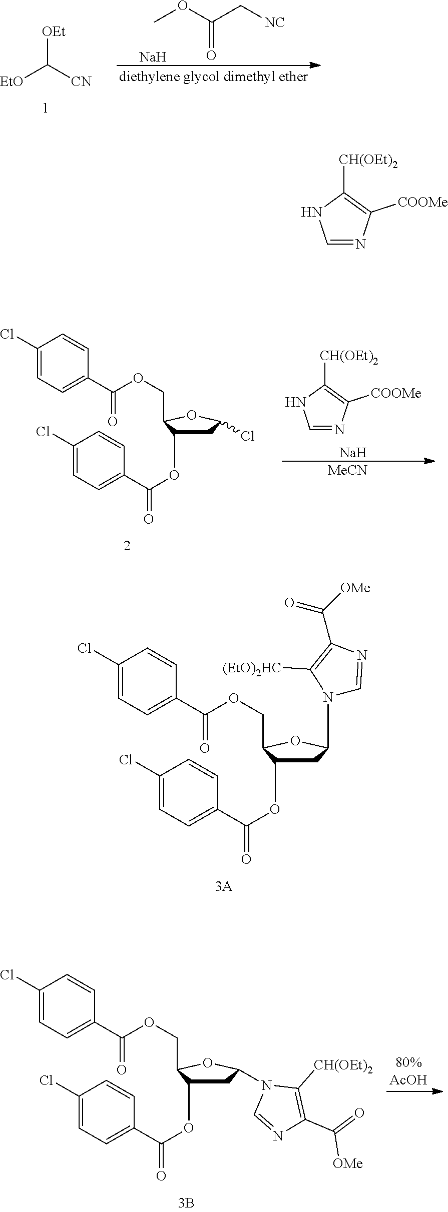

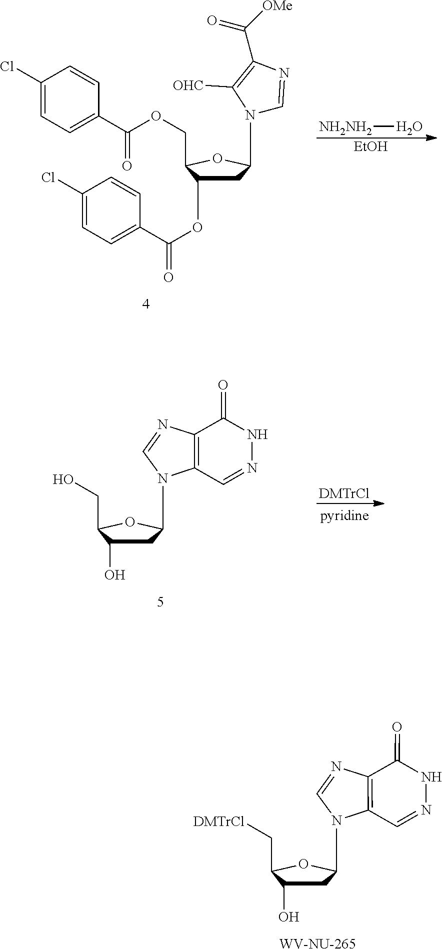

Step 1. To a solution of NaH (11.15 g, 278.73 mmol, 60% purity) in diethylene glycol dimethyl ether (300 mL) was added dropwise 3-oxopentanenitrile (15.04 g, 154.85 mmol) and 2,2-diethoxyacetonitrile (20 g, 154.85 mmol) in diethylene glycol dimethyl ether (50 mL) at 0° C. about 0.5 h. After addition, the resulting mixture was stirred at 80° C. for 1h. TLC indicated 2,2-diethoxyacetonitrile was consumed completely and one new spot formed. The reaction was clean according to TLC. The reaction mixture was partitioned between DCM 1000 mL and H₂O 500 mL. The organic phase was separated, washed with brine 300 mL, dried over Na₂SO₄, filtered and concentrated under reduced pressure to give a residue. The crude product was triturated with Petroleum Ether: Ethyl acetate=10:1 at 15° C. for 30 min. Methyl 5-(diethoxymethyl)-1H-imidazole-4-carboxylate (22 g, 62.25% yield) was obtained as a yellow solid.

Step 2. To a solution of methyl 5-(diethoxymethyl)-1H-imidazole-4-carboxylate (20 g, 87.63 mmol) in MeCN (500 mL) was added dropwise NaH (4.21 g, 105.15 mmol, 60% purity) at 0° C. After addition, the mixture was stirred at this temperature for 0.5 h and then (2R,3S)-5-chloro-2-(((4-chlorobenzoyl)oxy)methyl)tetrahydrofuran-3-yl 4-chlorobenzoate (37.65 g, 87.63 mmol) was added dropwise at 15° C. The resulting mixture was stirred at 60° C. for 2 hr. TLC indicated (2R,3S)-5-chloro-2-(((4-chlorobenzoyl)oxy)methyl)tetrahydrofuran-3-yl 4-chlorobenzoate was consumed completely and two new spot formed. The reaction was clean according to TLC. The reaction mixture was concentrated under reduced pressure to remove MeCN. The reaction mixture was quenched by addition H₂O 500 mL at 15° C. and extracted with EtOAc 1500 mL, the combined organic layers were washed with brine 200 mL dried over Na₂SO₄, filtered and concentrated under reduced pressure to give a residue. The residue was purified by column chromatography (SiO₂, Petroleum ether/Ethyl acetate=100/1 to 0/1). Methyl 1-((2R,4S,5R)-4-((4-chlorobenzoyl)oxy)-5-(((4-chlorobenzoyl)oxy)methyl)tetrahydrofuran-2-yl)-5-(diethoxymethyl)-1H-imidazole-4-carboxylate (20 g, 36.73% yield) was obtained as a white solid.

Step 3. To a solution of methyl 1-((2R,4S,5R)-4-((4-chlorobenzoyl)oxy)-5-(((4-chlorobenzoyl)oxy)methyl)tetrahydrofuran-2-yl)-5-(diethoxymethyl)-1H-imidazole-4-carboxylate (17 g, 27.35 mmol) in AcOH (30 mL)/H2O (120 mL). The mixture was stirred at 15° C. for 14 hr. The reaction was clean according to TLC. The resulting solid was isolated by filtration, washed and dried under vacuum to give residue. The residue was without further purification. Methyl 1-((2R,4S,5R)-4-((4-chlorobenzoyl)oxy)-5-(((4-chlorobenzoyl)oxy)methyl)tetrahydrofuran-2-yl)-5-formyl-1H-imidazole-4-carboxylate (14.97 g, 100.00% yield) was obtained as a white solid. ¹H NMR (CHLOROFORM-d, 400 MHz): δ=10.29 (s, 1H), 8.35-8.45 (m, 1H), 8.03 (d, J=8.5 Hz, 2H), 7.89 (d, J=8.5 Hz, 2H), 7.65 (d, J=8.6 Hz, 2H), 7.57 (d, J=8.6 Hz, 2H), 6.70 (t, J=6.5 Hz, 1H), 5.60-5.68 (m, 1H), 4.59-4.77 (m, 3H), 3.84-3.93 (m, 3H), 2.89 (ddd, J=14.5, 6.1, 2.9 Hz, 1H), 2.72 ppm (dt, J=14.4, 7.0 Hz, 1H).

Step 4. To a solution of methyl 1-((2R,4S,5R)-4-((4-chlorobenzoyl)oxy)-5-(((4-chlorobenzoyl)oxy)methyl)tetrahydrofuran-2-yl)-5-formyl-1H-imidazole-4-carboxylate (13 g, 23.75 mmol) in EtOH (300 mL) was added NH₂NH₂·H₂O (6.99 g. 118.76 mmol, 85% purity). The mixture was stirred at 80° C. for 72 hr. The reaction was clean according to TLC. The resulting solid was filtered, washed with ethanol and dried under high vacuum to give residue as off white solid. The residue was without further purification. 1-((2R,4S,5R)-4—Hydroxy-5-(hydroxymethyl)tetrahydrofuran-2-yl)-1,5-dihydro-4H-imidazo[4,5-d]pyridazin-4-one (3.5 g, 58.42% yield) was obtained as a white solid. ¹H NMR (DMSO-d6, 400 MHz): δ=12.73 (br s, 1H), 8.50-8.71 (m, 2H), 6.38 (br t, J=6.2 Hz, 1H), 5.38 (br d, J=3.3 Hz, 1H), 5.05 (br s, 1H), 4.32-4.46 (m, 1H), 3.91 (br s, 1H), 3.58 (br d, J=4.0 Hz, 2H), 2.37 ppm (br d, J=12.8 Hz, 1H).

Step 5. To a solution of 1-((2R,4S,5R)-4-hydroxy-5-(hydroxymethyl)tetrahydrofuran-2-yl)-1,5-dihydro-4H-imidazo[4,5-d]pyridazin-4-one (4.5 g, 17.84 mmol, 1 eq) in PY (100 mL) was added DMTrCl (6.65 g, 19.63 mmol) and DMAP (1.09 g, 8.92 mmol). The mixture was stirred at 15° C. for 2 hr. The reaction mixture was partitioned between H₂O 300 mL and EtOAc 1000 mL. The organic phase was separated, washed with brine 300 mL, dried over Na₂SO₄, filtered and concentrated under reduced pressure to give a residue. The residue was purified by column chromatography (SiO₂, Dichloromethane: Methanol=100/1 to 0/1). Compound WV-NU-265 (6.5 g, 65.69% yield) was obtained as a white solid. ¹HNMR (DMSO-d₆, 400 MHz): δ=12.77 (s, 1H), 8.53 (s, 1H), 8.50 (s, 1H), 7.11-7.28 (m, 9H), 6.79 (dd, J=8.9, 6.4 Hz, 4H), 6.42 (t, J=6.0 Hz, 1H), 5.46 (d, J=4.8 Hz, 1H), 4.41 (quin, J=5.3 Hz, 1H), 3.98-4.06 (m, 1H), 3.72-3.83 (m, 3H), 3.35 (s, 2H), 3.06-3.15 (m, 2H), 2.67-2.81 (m, 1H), 2.42-2.49 ppm (m, 1H); LCMS (M−H+)=553, LCMS purity: 96.1%.

### Synthesis of 1-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-1,5-dihydro-4H-imidazo[4,5-d]pyridazin-4-one

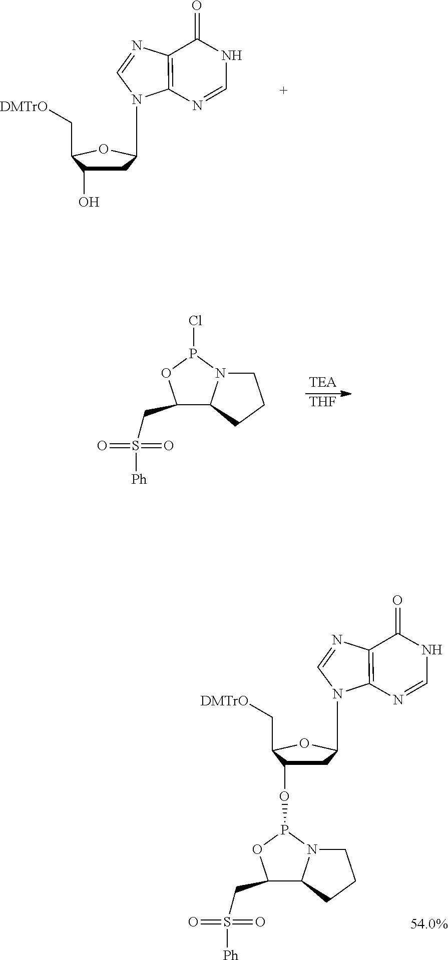

To a slurry of dry 3-[(2R,4R,5R)-5-[[bis(4-methoxyphenyl)-phenyl-methoxy]methyl]-4-hydroxy-tetrahydrofuran-2-yl]-6H-imidazo[4,5-d]pyridazin-7-one (3.00 g, 5.41 mmol) in THF (45 mL) was added triethylamine (1.66 mL, 11.9 mmol). (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.62 mL, 8.66 mmol) was added fast dropwise. The resulting slurry was stirred at rt for 6 hr. TLC and LCMS showed that the r×n was complete. The reaction was quenched by water (49 μL). Anhydrous MgSO₄ (649 mg) was added. The mixture was filtered through celite, and the filtrate was concentrated. The resulting crude product was purified by normal phase column chromatography applying 0-40% ACN in EtOAc as the gradient to afford the title compound as a white foam (2.45 g, 54.0% yield). ¹H NMR (600 MHz, Chloroform-d) δ 10.56 (s, 1H), 8.33 (s, 1H), 8.03 (s, 1H), 7.92-7.87 (m, 2H), 7.62-7.57 (m, 1H), 7.51-7.46 (m, 2H), 7.35-7.31 (m, 2H), 7.28-7.22 (m, 6H), 7.22-7.19 (m, 1H), 6.84-6.77 (m, 4H), 6.22 (dd, J=8.2, 5.6 Hz, 1H), 5.10 (dt, J=7.2, 5.4 Hz, 1H), 4.91-4.86 (m, 1H), 4.24 (td, J=3.7, 2.0 Hz, 1H), 3.775 (s, 3H), 3.772 (s, 3H), 3.67 (ddt, J=10.0, 7.0, 5.6 Hz, 1H), 3.58-3.50 (m, 1H), 3.52-3.47 (m, 1H), 3.39 (dd, J=14.5, 5.1 Hz, 1H), 3.35 (dd, J=10.6, 3.7 Hz, 1H), 3.31 (dd, J=10.6, 3.8 Hz, 1H), 3.19 (tdd, J=10.4, 8.8, 4.0 Hz, 1H), 2.70-2.60 (m, 2H), 1.93 (dddt, J=14.9, 10.8, 7.9, 5.2 Hz, 1H), 1.87-1.77 (m, 1H), 1.66 (tdd, J=9.0, 7.7, 6.6, 3.9 Hz, 1H), 1.13 (dtd, J=11.6, 10.3, 8.5 Hz, 1H); ³¹P NMR (243 MHz, Chloroform-d) δ 154.40; MS (ESI), 876.05 [M+K]⁺.

### Synthesis of 1-((2R,3S,4R,5R)-5-((bis(4-methoxyphenyl)(phenyl)methoxy)methyl)-3-fluoro-4-hydroxytetrahydrofuran-2-yl)pyrimidine-2,4(1H,3H)-dione (WV-NU-284)

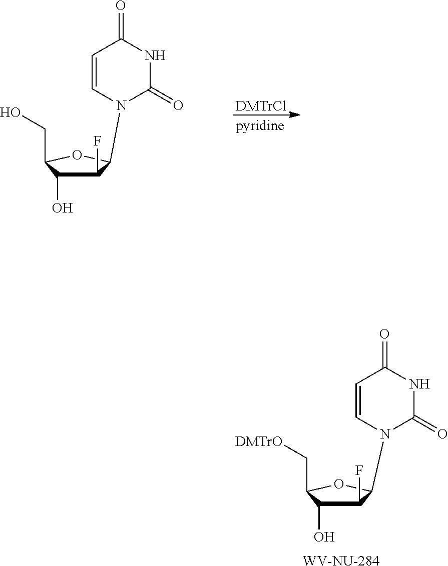

To a solution of 1-((2R,3S,4R,5R)-3-fluoro-4-hydroxy-5-(hydroxymethyl)tetrahydrofuran-2-yl)pyrimidine-2,4(1H,3H)-dione (15 g, 60.93 mmol) in pyridine (350 mL) was added DMTCl (22.71 g, 67.02 mmol). The mixture was stirred at 20° C. for 12 hr. The reaction mixture was concentrated under reduced pressure to remove Pyridine. The residue was diluted with H₂O 300 mL and extracted with Ethyl acetate (100 mL*3). The combined organic layers were washed with salt water 200 mL, dried over Na₂SO₄, filtered and concentrated under reduced pressure to give a residue. The residue was purified by column chromatography (SiO₂, Petroleum ether/Ethyl acetate=1/0 to 0/1). Compound WV-NU-284 (29.5 g, 88.26% yield) was obtained as a white solid. ¹HNMR (400 MHz, DMSO-d₆) δ=11.51 (s, 1H), 7.52 (dd, J=1.1, 8.2 Hz, 1H), 7.43-7.38 (m, 2H), 7.36-7.20 (m, 7H), 6.91 (d, J=8.9 Hz, 4H), 6.17 (dd, J=4.3, 15.6 Hz, 1H), 6.00 (br d, J=4.9 Hz, 1H), 5.49 (d, J=8.1 Hz, 1H), 5.18-4.95 (m, 1H), 4.33-4.20 (m, 1H), 4.01-3.95 (m, 1H), 3.74 (s, 6H), 3.28 (br d, J=4.4 Hz, 2H); LCMS (M−H+): 547.3, LCMS purity: 97.8%; TLC (Ethyl acetate:Petroleum ether=2:1), Rf=0.4.

### Synthesis of 1-((2R3S,4R,5R)-5-((bis(4-methoxyphenyl)(phenyl)methoxy)methyl)-3-fluoro-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

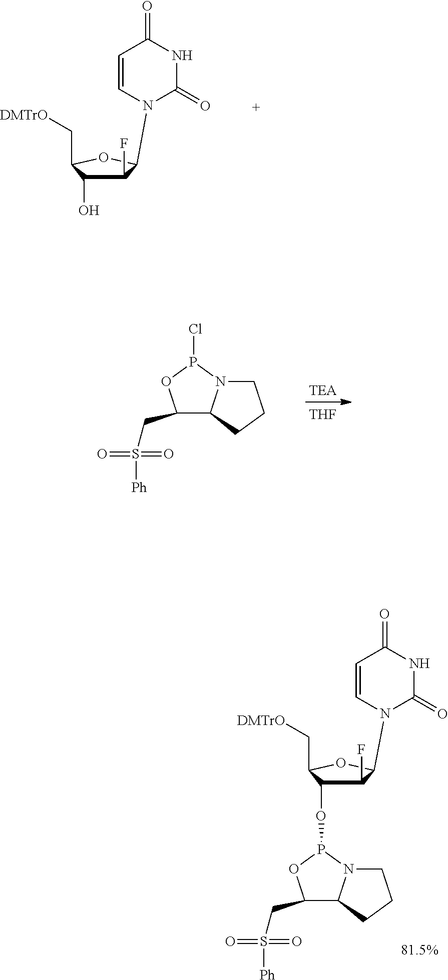

To a solution of dry 1-[(2R,3R,5R)-5-[[bis(4-methoxyphenyl)-phenyl-methoxy]methyl]-3-fluoro-4-hydroxy-tetrahydrofuran-2-yl]pyrimidine-2,4-dione (15.0 g, 27.3 mmol) in THF (90 mL) was added triethylamine (8.38 mL, 60.2 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 48.6 mL, 43.8 mmol) was added fast dropwise. The ice/water bath was removed. The resulting slurry was stirred at rt for 2 hr. TLC and LCMS showed that the r×n was complete. The reaction was quenched by water (246 μL). Anhydrous MgSO₄ (3.28 g) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a white foam (18.5 g, 81.5% yield). ¹H NMR (600 MHz, Chloroform-d) δ 8.70 (s, 1H), 7.92-7.86 (m, 2H), 7.64-7.60 (m, 1H), 7.55 (dd, J=8.2, 1.9 Hz, 1H), 7.53-7.49 (m, 2H), 7.45-7.40 (m, 2H), 7.36-7.26 (m, 6H), 7.25-7.22 (m, 1H), 6.87-6.80 (m, 4H), 6.19 (dd, J=18.7, 3.6 Hz, 1H), 5.56 (d, J=8.2 Hz, 1H), 5.03 (ddd, J=51.2, 3.6, 1.7 Hz, 1H), 5.00 (q, J=6.4 Hz, 1H), 4.76 (dddd, J=19.4, 9.2, 4.2, 1.8 Hz, 1H), 3.97 (q, J=4.2 Hz, 1H), 3.79 (s, 6H), 3.63 (dq, J=9.9, 5.9 Hz, 1H), 3.52-3.40 (m, 3H), 3.39-3.31 (m, 2H), 3.04 (tdd, J=10.4, 8.9, 4.1 Hz, 1H), 1.94-1.85 (m, 1H), 1.78 (dddd, J=19.6, 13.0, 9.8, 7.7 Hz, 1H), 1.67 (dddd, J=9.5, 7.4, 5.4, 2.6 Hz, 1H), 1.11 (dtd, J=11.8, 10.2, 8.5 Hz, 1H); ³¹P NMR (243 MHz, Chloroform-d) δ 151.51; ¹⁹F NMR (565 MHz, Chloroform-d) δ −197.06; MS (ESI), 830.18 [M−H]⁻.

### Synthesis of 4-amino-i-((2R,4S,5R)-5-((bis(4-methoxyphenyl)(phenyl)methoxy)methyl)-4-hydroxytetrahydrofuran-2-yl)-1,3,5-triazin-2(1H)-one (WV-274A)

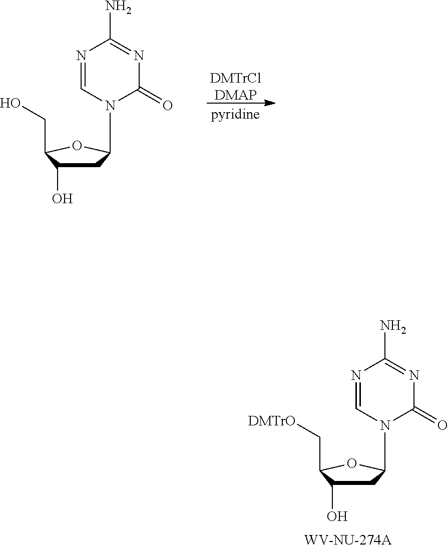

To a solution of 4-amino-i-((2R,4S,5R)-4-hydroxy-5-(hydroxymethyl)tetrahydrofuran-2-yl)-1,3,5-triazin-2(1H)-one (5.00 g, 21.91 mmol) in pyridine (200 mL) was added DMT-C1 (7.42 g, 21.91 mmol) and DMAP (1.34 g, 10.96 mmol) in N₂. The mixture was stirred at 20° C. for 2 hr. The reaction mixture was filtered and concentrated under reduced pressure to give a residue. The residue was purified by column chromatography (SiO₂, Petroleum ether/Ethyl acetate=10:1 to 0:1 to Ethyl acetate: Methanol=1:1, 5% TEA). Compound WV-NU-274A (17 g, 91.40% yield) was obtained as a yellow solid. ¹H NMR (400 MHz, DMSO-d6) δ=8.31 (s, 1H), 7.54 (s, 2H), 7.42-7.35 (m, 2H), 7.34-7.18 (m, 7H), 6.88 (d, J=8.6 Hz, 4H), 6.04 (t, J=6.4 Hz, 1H), 5.29 (d, J=4.6 Hz, 1H), 4.23 (quin, J=4.8 Hz, 1H), 3.96-3.90 (m, 1H), 3.74 (s, 6H), 3.32 (s, 1H), 3.26-3.13 (m, 2H), 2.24 (t, J=5.9 Hz, 2H); TLC: Ethyl acetate: Methanol=5:1, Rf=0.41; LCMS (M−H+): 529.3, LCMS purity: 98.27%.

### Synthesis of 4-amino-i-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-1,3,5-triazin-2(1H)-one

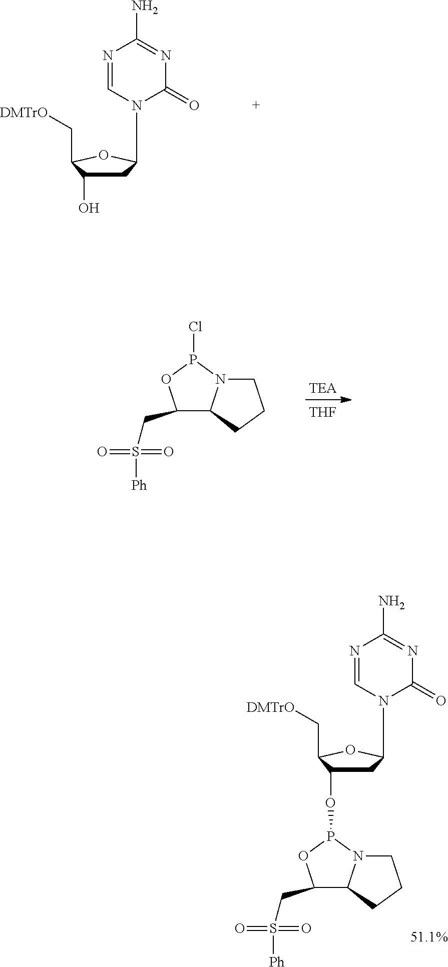

To a solution of dry 4-amino-1-[(2R,4R,5R)-5-[[bis(4-methoxyphenyl)-phenyl-methoxy]methyl]-4-hydroxy-tetrahydrofuran-2-yl]-1,3,5-triazin-2-one (3.00 g, 5.65 mmol) in THF (18 mL) was added triethylamine (1.97 mL, 14.1 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.75A4 in THF, 13.6 mL, 10.2 mmol) was added fast dropwise. The ice/water bath was removed. The resulting slurry was stirred at rt for 3 hr. TLC and LCMS showed that the r×n was complete. The reaction was quenched by water (51 L). Anhydrous MgSO₄ (678 mg) was added. The mixture was filtered through celite, and the filtrate was concentrated. The resulting crude product was purified by normal phase column chromatography applying 0-40% ACN in EtOAc (each mobile phase contained 2.5% triethylamine) as the gradient to afford the title compound as a yellowish off-white foam (2.35 g, 51.1% yield). ¹H NMR (600 MHz, Chloroform-d) δ 8.40 (s, 1H), 7.88 (dq, J=8.6, 1.5 Hz, 2H), 7.62-7.59 (m, 1H), 7.52-7.48 (m, 2H), 7.40-7.36 (m, 2H), 7.32-7.25 (m, 6H), 7.24-7.20 (m, 1H), 6.87-6.81 (m, 4H), 6.12 (t, J=6.4 Hz, 1H), 6.11 (s, 1H), 5.60 (s, 1H), 4.95 (dt, J=6.9, 5.6 Hz, 1H), 4.69 (ddq, J=10.1, 6.9, 3.5 Hz, 1H), 3.78 (s, 6H), 3.76-3.70 (m, 1H), 3.61 (dq, J=9.9, 5.9 Hz, 1H), 3.52-3.43 (m, 2H), 3.39-3.30 (m, 3H), 3.14 (tdd, J=10.3, 8.8, 4.1 Hz, 1H), 2.74 (ddd, J=14.0, 6.1, 3.2 Hz, 1H), 2.28-2.22 (m, 1H), 1.90-1.84 (m, 1H), 1.78 (ddd, J=9.6, 7.8, 4.9 Hz, 1H), 1.63 (dtt, J=9.9, 5.4, 2.7 Hz, 1H), 1.10 (dtd, J=11.7, 10.2, 8.4 Hz, 1H); ³¹P NMR (243 MHz, Chloroform-d) δ 153.71; MS (ESI), 852.45 [M+K]⁺.

### Synthesis of 3-((2R,3S,4R,5R)-5-((bis(4-methoxyphenyl)(phenyl)methoxy)methyl)-3-fluoro-4-hydroxytetrahydrofuran-2-yl)pyrimidine-2,4(1H,3H)-dione (WV-NU-267) and 3-((2S,3S,4R,5R)-5-((bis(4-methoxyphenyl)(phenyl)methoxy)methyl)-3-fluoro-4-hydroxytetrahydrofuran-2-yl)pyrimidine-2,4(1H,3H)-dione (WV-267A)

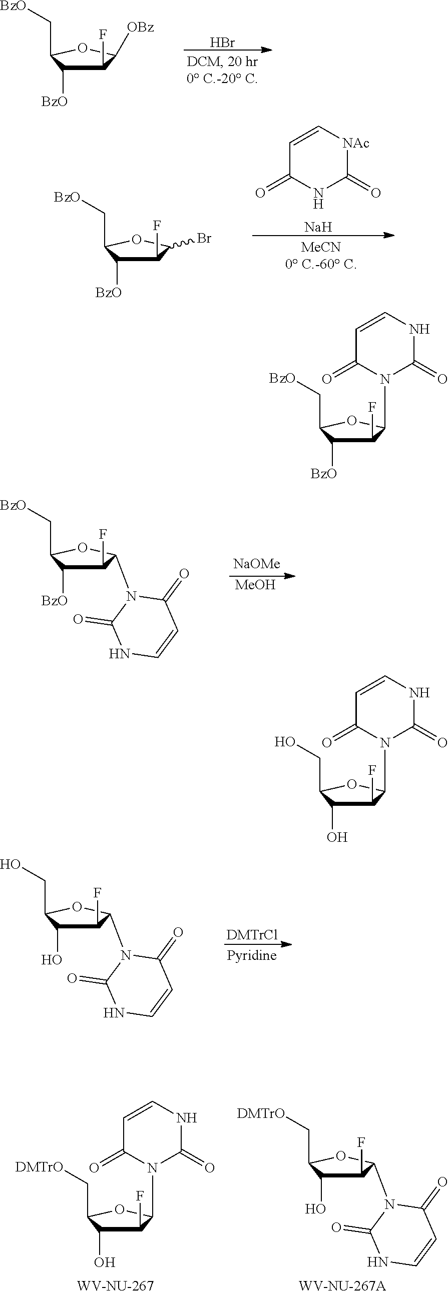

Step 1. For two batches: To a solution of (2S,3S,4R,5R)-5-((benzoyloxy)methyl)-3-fluorotetrahydrofuran-2,4-diyl dibenzoate (75 g, 161.49 mmol) in DCM (1500 mL) was added hydrogen bromide (158.37 g, 645.94 mmol) at 0° C. in N₂. The mixture was stirred at 0-20° C. for 20 hr. TLC indicated compound 1 was consumed completely and one new spot formed. The reaction mixture of two batches poured into 500 mL of ice water, and the organic phase was separated, washed with water (5×200 mL), dried over MgSO₄, and evaporated under reduced pressure, and then the crude product to remove remaining acetic acid and dried in vacuum. ((2R,3R,4S)-3-(benzoyloxy)-5-bromo-4-fluorotetrahydrofuran-2-yl)methyl benzoate (136 g, crude) was obtained as a yellow oil.

Step 2. For four batches: To a solution of 1-acetylpyrimidine-2,4(1H,3H)-dione (12.38 g, 80.33 mmol) in ACN (2000 mL) was added NaH (6.43 g, 160.66 mmol) at 0° C. for 0.5 hr, and then added ((2R,3R,4S)-3-(benzoyloxy)-5-bromo-4-fluorotetrahydrofuran-2-yl)methyl benzoate (34 g, 80.33 mmol). The mixture was stirred at 0-60° C. for 3 hr. TLC indicated ((2R,3R,4S)-3-(benzoyloxy)-5-bromo-4-fluorotetrahydrofuran-2-yl)methyl benzoate was consumed completely and two new spots formed. The reaction mixture of four batches were filtered and concentrated under reduced pressure to give a residue. The residue was purified by column chromatography (SiO₂, Petroleum ether:Ethyl acetate=1:0 to 1:1). ((2R,3R,4S,5R)-3-(benzoyloxy)-5-(2,6-dioxo-3,6-dihydropyrimidin-1(2H)-yl)-4-fluorotetrahydrofuran-2-yl)methyl benzoate (13 g, 8.90% yield) was obtained as a white solid. ¹H NMR (400 MHz, DMSO-d6) δ=11.36-11.30 (m, 1H), 8.00 (d, J=7.5 Hz, 2H), 7.87 (d, J=7.5 Hz, 2H), 7.73-7.67 (m, 1H), 7.62-7.51 (m, 4H), 7.44-7.36 (m, 2H), 6.85 (dd, J=4.6, 8.0 Hz, 1H), 6.32-6.20 (m, 1H), 5.96-5.75 (m, 1H), 5.65 (d, J=7.6 Hz, 1H), 4.71-4.53 (m, 2H), 4.45-4.34 (m, 1H). TLC: Petroleum ether:Ethyl acetate=1:1.5, Rf=0.17. ((2R,3R,4S,5S)-3-(benzoyloxy)-5-(2,6-dioxo-3,6-dihydropyrimidin-1(2H)-yl)-4-fluorotetrahydrofuran-2-yl)methyl benzoate (49 g, 33.56% yield) was obtained as a white solid. ¹H NMR (400 MHz, DMSO-d6) δ=11.40 (br s, 1H), 8.03 (br d, J=7.3 Hz, 2H), 7.96 (br d, J=7.1 Hz, 1H), 7.75-7.61 (m, 2H), 7.60-7.47 (m, 5H), 6.69-6.54 (m, 1H), 6.21-6.01 (m, 1H), 5.92-5.77 (m, 1H), 5.68 (d, J=7.6 Hz, 1H), 4.94 (td, J=3.7, 7.4 Hz, 1H), 4.62-4.47 (m, 2H); TLC: Petroleum ether:Ethyl acetate=1:1.5, Rf₂=0.11.

Step 3. To a solution of ((2R,3R,4S,5R)-3-(benzoyloxy)-5-(2,6-dioxo-3,6-dihydropyrimidin-1(2H)-yl)-4-fluorotetrahydrofuran-2-yl)methyl benzoate (13 g, 28.61 mmol) in MeOH (150 mL) was added NaOMe (3.09 g, 57.22 mmol). The mixture was stirred at 25° C. for 2 hr. The reaction was added NH₄Cl (3.1 g) and stirred at room temperature for 5 min, and then vacuum concentration. The residue was purified by column chromatography (SiO₂, Petroleum ether:Ethyl acetate=1:1 to 0:1 to Ethyl acetate: MeOH=1:1). 3-((2R,3S,4R,5R)-3-Fluoro-4-hydroxy-5-(hydroxymethyl)tetrahydrofuran-2-yl)pyrimidine-2,4(1H,3H)-dione (6.6 g, 94.29% yield) was obtained as a white solid. ¹H NMR (400 MHz, DMSO-d6) δ=11.14 (br s, 1H), 7.46 (d, J=7.6 Hz, 1H), 6.61 (dd, J=6.0, 7.9 Hz, 1H), 5.57 (d, J=7.6 Hz, 1H), 5.28 (dd, J=6.3, 7.8 Hz, 1H), 5.14 (dd, J=6.2, 7.8 Hz, 1H), 4.61-4.46 (m, 2H), 3.67-3.56 (m, 3H); LCMS (M+H⁺): 269.2, purity: 96.9%; TLC: Petroleum ether:Ethyl acetate=0:1, Rf=0.21.

Step 4. To a solution of 3-((2R,3S,4R,5R)-3-fluoro-4-hydroxy-5-(hydroxymethyl)tetrahydrofuran-2-yl)pyrimidine-2,4(1H,3H)-dione (6.6 g, 26.81 mmol) in Py (5 mL) was added DMTCl (9.08 g, 26.81 mmol) in N₂. The mixture was stirred at 25° C. for 6 hr. The reaction mixture was filtered and concentrated under reduced pressure to give a residue. The residue was purified by column chromatography (SiO₂, Petroleum ether:Ethyl acetate=0:1 to 1:0 to Ethyl acetate: MeOH=5:1). Compound WV-NU-267 (13 g, 88.40% yield) was obtained as a white solid. ¹HNMR (400 MHz, DMSO-d6) δ=11.15 (br s, 1H), 7.49 (d, J=7.5 Hz, 1H), 7.39 (d, J=7.4 Hz, 2H), 7.32-7.15 (m, 7H), 6.85 (t, J=8.7 Hz, 4H), 6.71 (dd, J=5.0, 8.0 Hz, 1H), 5.64-5.54 (m, 2H), 5.35-5.15 (m, 1H), 4.54-4.39 (m, 1H), 3.85 (br t, J =7.9 Hz, 1H), 3.73 (d, J=1.6 Hz, 6H), 3.46 (t, J=9.2 Hz, 1H), 3.09-3.03 (m, 1H); LCMS (M−H⁺): 547.3, purity: 95.92%; TLC: Petroleum ether:Ethyl acetate=0:1, Rf=0.48.

Step 5. To a solution of ((2R,3R,4S,5S)-3-(benzoyloxy)-5-(2,6-dioxo-3,6-dihydropyrimidin-1(2H)-yl)-4-fluorotetrahydrofuran-2-yl)methyl benzoate (49 g, 107.83 mmol) in MeOH (500 mL) was added NaOMe (12.82 g, 237.23 mmol). The mixture was stirred at 25° C. for 2 hr. The reaction was added NH₄C1 (12 g) and stirred at room temperature for 5 min, and then vacuum concentration. The residue was purified by column chromatography (SiO₂, Petroleum ether:Ethyl acetate=10:1 to 0:1 to Ethyl acetate: MeOH =10:1). 3-((2S,3S,4R,5R)-3-Fluoro-4-hydroxy-5-(hydroxymethyl)tetrahydrofuran-2-yl)pyrimidine-2,4(1H,3H)-dione (25 g, 96.15% yield) was obtained as a yellow solid. ¹HNMR (400 MHz, DMSO-d6) δ=11.80-10.71 (m, 1H), 7.49 (d, J=7.6 Hz, 1H), 6.42-6.31 (m, 1H), 5.83-5.75 (m, 1H), 5.68-5.59 (m, 1H), 4.91-4.66 (m, 1H), 4.34-4.18 (m, 2H), 3.66-3.54 (m, 1H), 3.42 (br dd, J=4.0, 12.4 Hz, 1H); LCMS (M+H⁺): 247.2, purity: 98.26%; TLC: Petroleum ether:Ethyl acetate=0:1, Rf=0.21.

Step 6. To a solution of 3-((2S,3S,4R,5R)-3-fluoro-4-hydroxy-5-(hydroxymethyl)tetrahydrofuran-2-yl)pyrimidine-2.4(1H,3H)-dione (25 g, 101.55 mmol) in Py (500 mL) was added DMTrCl (34.41 g, 101.55 mmol) in N₂. The mixture was stirred at 25° C. for 6 hr. The reaction mixture was filtered and concentrated under reduced pressure to give a residue. The residue was purified by column chromatography (SiO₂, Petroleum ether:Ethyl acetate=20:1 to 0:1 to Ethyl acetate: MeOH=5:1). Compound WV-NU-267A (34 g, 61.04% yield) was obtained as a yellow solid. ¹H NMR (400 MHz, DMSO-d6) δ=11.81-10.78 (m, 1H), 7.53 (d, J=7.6 Hz, 1H), 7.43-7.37 (m, 2H), 7.36-7.20 (m, 7H), 6.90 (d, J=8.9 Hz, 4H), 6.50-6.40 (m, 1H), 5.87-5.77 (m, 1H), 5.70-5.62 (m, 1H), 4.48-4.40 (m, 1H), 4.39-4.26 (m, 1H), 3.75 (s, 6H), 3.20 (br d, J=9.9 Hz, 1H), 3.05 (dd, J=5.4, 10.6 Hz, 1H); LCMS (M−H⁺): 547.3, purity: 93.07%; TLC: Petroleum ether: Ethyl acetate=0:1, Rf=0.48.

### Synthesis of 3-((2R,4S,5R)-5-((bis(4-methoxyphenyl)(phenyl)methoxy)methyl)-4-hydroxetrahydrofuran-2-yl)-2,4-dioxo-1,2,3,4-tetrahydropyrimidine-5-carbonitrile (WV-272)

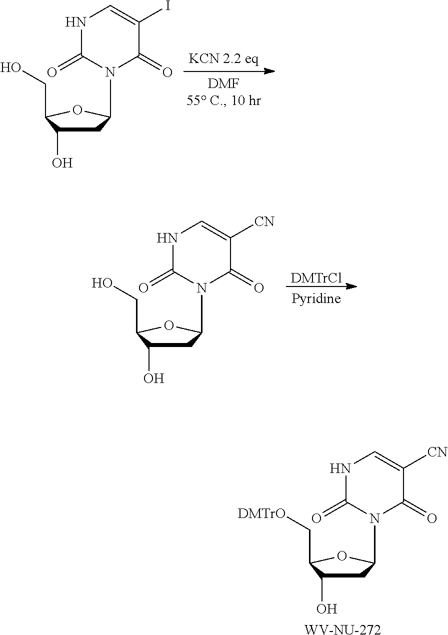

Step 1. To a solution of 3-((2R,4S,5R)-4-hydroxy-5-(hydroxymethyl)tetrahydrofuran-2-yl)-5-iodopyrimidine-2,4(1H,3H)-dione (3.6 g, 10.17 mmol) in DMF (60 mL) was added KCN (1.51 g, 23.19 mmol, 993.42 uL) and 1,4,7,10,13,16-hexaoxacyclooctadecane (10.75 g, 40.67 mmol). The mixture was stirred at 55° C. for 15 hr. The mixture was added sat. NaHCO₃ solution to adjust pH 9. The mixture was diluted with H₂O 300 mL and extracted with EtOAc 200 mL*3. The combined organic layers were dried over Na₂SO₄, filtered and concentrated under reduced pressure to give a residue. The residue was purified by column chromatography (SiO₂, DCM: MeOH=100: 0 to 5:1). 3-((2R,4S,5R)-4-hydroxy-5-(hydroxymethyl)tetrahydrofuran-2-yl)-2,4-dioxo-1,2,3,4-tetrahydropyrimidine-5-carbonitrile (2.0 g, 77.69% yield) was obtained as yellow oil. LCMS (M−H⁺):252.1; TLC: DCM: MeOH=5:1, Rf=0.52.

Step 2. For two batches: To a solution of 3-((2R,4S,5R)-4-hydroxy-5-(hydroxymethyl)tetrahydrofuran-2-yl)-2,4-dioxo-1,2,3,4-tetrahydropyrimidine-5-carbonitrile (2.5 g, 9.87 mmol) in PYRIDINE (30 mL) was added DMTCl (3.35 g, 9.87 mmol). The mixture was stirred at 15° C. for 1 hr. 10 mL MeOH was added to the mixture and the reaction mixture was concentrated under reduced pressure to remove solvent. The residue was purified by column chromatography (SiO2, Petroleum ether:Ethyl acetate =100:1 to 0:1, 5% TEA). Compound WV-NU-272 (9 g, 82.06% yield) was obtained as yellow solid. ¹H NMR (400 MHz, DMSO-d6) δ=7.38 (d, J=7.4 Hz, 2H), 7.31-7.16 (m, 7H), 6.84 (t, J=9.3 Hz, 4H), 6.55 (dd, J=4.2, 9.1 Hz, 1H), 6.26 (s, 1H), 5.05 (br d, J=3.0 Hz, 1H), 4.23 (br d, J=6.5 Hz, 1H). 3.87-3.78 (m, 1H), 3.73 (d, J=1.6 Hz, 6H), 3.24-3.17 (m, 1H), 3.13-3.05 (m, 2H), 2.56 (ddd, J=4.3, 8.3, 12.9 Hz, 1H), 1.99-1.94 (m, 1H); LCMS (M−H+):554.3, purity: 93.70%; TLC: Petroleum ether:Ethyl acetate=1:2, Rf=0.45.

### Synthesis of (S)-3-(3-(bis(4-methoxyphenyl)(phenyl)methoxy)-2-hydroxypropyl)pyrimidine-2,4(1H,3H)-dione (WV-239)

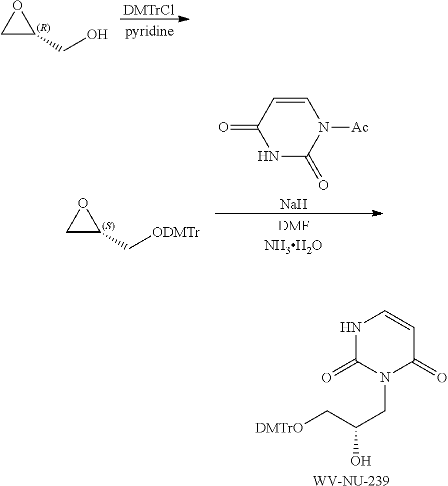

Step 1. Two batches were set up: To a solution of (R)-oxiran-2-ylmethanol (25 g, 337.48 mmol, 22.32 mL) in PYRIDINE (1250 mL) was added DMTCl (125.78 g, 371.23 mmol). The mixture was stirred at 25° C. for 10 hr. 2 Batches were mixed. The mixture was diluted with H₂O 2000 mL and exacted with Ethyl acetate 2000 mL*3. The organic phase was dried over Na₂SO₄, filtered and concentrated under reduced pressure to give a residue. The residue was purified by column chromatography (SiO₂, Petroleum ether/Ethyl acetate=100/1 to 5/1). (S)-2-((Bis(4-methoxyphenyl)(phenyl)methoxy)methyl)oxirane (160 g, crude) was obtained as yellow oil. TLC: Petroleum ether:Ethyl acetate=3:1, Rf=0.3.

Step 2. Five batches were set up: To a solution of 1-acetylpyrimidine-2,4(1H,3H)-dione (4.50 g, 29.22 mmol) in DMF (50 mL) was added NaH (223.14 mg, 5.58 mmol, 60% purity) and (S)-2-((bis(4-methoxyphenyl)(phenyl)methoxy)methyl)oxirane (10 g, 26.56 mmol). The mixture was stirred at 110° C. for 12 hr. Five batches were mixed. To a solution of mixture was added NH₃·H₂O (72.80 g, 519.32 mmol, 25% purity). The mixture was stirred at 60° C. for 12 hr. LCMS showed 14% of intermediate state remained. Several new peaks were shown on LCMS and 40% of desired compound was detected. The reaction mixture was concentrated under reduced pressure to remove DMF. The residue was diluted with H₂O 300 mL and extracted with Ethyl acetate (200 mL *2). The combined organic layers were washed with NaCl 300 mL, dried over Na₂SO₄, filtered and concentrated under reduced pressure to give a residue. The residue was purified by column chromatography (SiO₂, Petroleum ether/Ethyl acetate=1/0 to 0/1, Ethyl acetate/Methanol=1/0 to 50/1). Compound WV-NU-239 (5 g, 62.50% yield) was obtained as a yellow solid. ¹H NMR (400 MHz, DMSO-d6) δ=11.02 (br s, 1H), 7.42-7.36 (m, 3H), 7.32-7.22 (m. 7H), 6.91-6.85 (m, 4H), 5.54 (d, J=7.6 Hz, 1H), 4.96 (d, J=5.8 Hz, 1H), 4.02-3.95 (m, 1H), 3.89-3.80 (m, 1H), 3.74 (s, 7H), 2.99 (dd, J=5.6, 9.5 Hz, 1H), 2.87-2.81 (m, 1H); LCMS (M−H⁺):487.2, LCMS purity: 95.37%; TLC: Petroleum ether:Ethyl acetate=0:1, Rf=0.3.

### Synthesis of (R)-3-(3-(bis(4-methoxyphenyl)(phenyl)methoxy)-2-hvdroxypropyl)pyrimidine-2,4(1H,3H)-dione (WV-240)

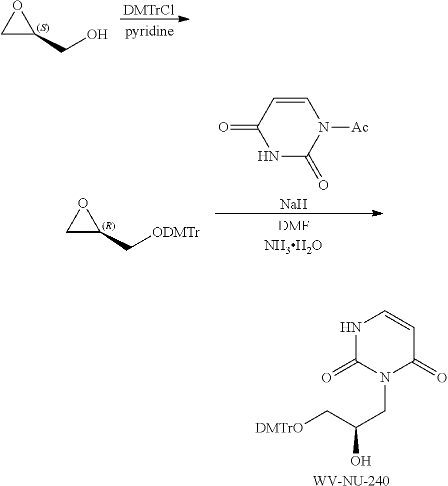

Step 1. To a solution of (S)-oxiran-2-ylmethanol (20 g, 269.98 mmol) in PYRIDINE (1000 mL) was added DMTCl (91.48 g, 269.98 mmol). The mixture was stirred at 20° C. for 12 hr. TLC indicated compound 1 was consumed completely and two new spots formed. The reaction was clean according to TLC. The reaction mixture was concentrated under reduced pressure to remove pyridine. The residue was purified by column chromatography (SiO₂, Petroleum ether/Ethyl acetate=1/0 to 0/1). (R)-2-((bis(4-methoxyphenyl)(phenyl)methoxy)methyl)oxirane (72 g, 70.84% yield) was obtained as a yellow oil. TLC: Petroleum ether:Ethyl acetate=3:1, Rf=0.3.

Step 2. Five batches: To a solution of 1-acetylpyrimidine-2,4(1H,3H)-dione (4.50 g, 29.22 mmol) in DMF (50 mL) was added NaH (223.08 mg, 5.58 mmol, 60% purity) and (R)-2-((bis(4-methoxyphenyl)(phenyl)methoxy)methyl)oxirane (10 g, 26.56 mmol). The mixture was stirred at 110° C. for 12 hr. Five batches were mixed. The mixture liquid was added NH₃·H₂O (364.04 g, 2.60 mol, 25% purity). The mixture was stirred at 60° C. for 12 hr. LCMS showed 18% of intermediate state remained. Several new peaks were shown on LCMS and 25% of desired compound WV-NU-240 was detected. The reaction mixture was concentrated under reduced pressure to remove DMF. The residue was diluted with H₂O 300 mL and extracted with ethyl acetate (200 mL *2). The combined organic layers were washed with NaCl 300 mL, dried over Na₂SO₄, filtered and concentrated under reduced pressure to give a residue. The residue was purified by column chromatography (SiO₂, Petroleum ether/Ethyl acetate=1/0 to 0/1, Ethyl acetate/Methanol=I/O to 50/1). Compound WV-NU-240 (2.5 g, 3.88% yield) was obtained as a white solid. ¹HNMR (400 MHz, DMSO-d6) 6=11.01 (s, 1H), 7.42-7.35 (m, 3H), 7.32-7.21 (m, 7H), 6.91-6.83 (m, 4H), 5.54 (d, J=7.6 Hz, 1H), 4.95 (d, J=5.7 Hz, 1H), 4.01-3.94 (m, 1H), 3.88-3.81 (m, 1H), 3.74 (s, 7H), 2.99 (dd, J=5.7, 9.4 Hz, 1H), 2.86-2.79 (m, 1H); LCMS (M−H⁺):487.2, 99.36% purity; TLC: Petroleum ether:Ethyl acetate=0:1, Rf=0.3.

### Synthesis of 9-((2R,4S,5R)-5-((bis(4-methoxyphenyl)(phenyl)methoxy)methyl)-4-hydroxytetrahydrofuran-2-yl)-6-(dimethylamino)-7,9-dihydro-8H-purin-8-one (WV-232)

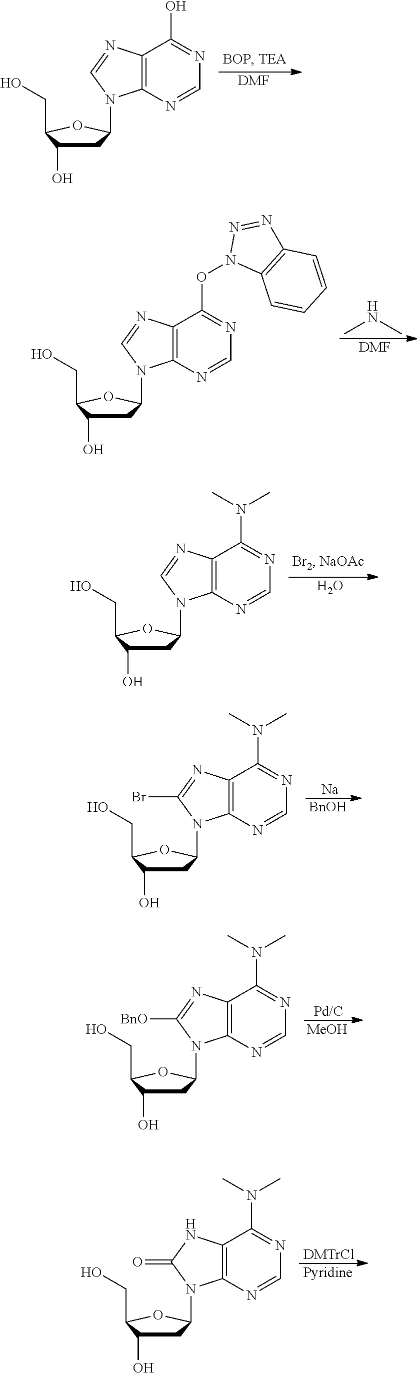

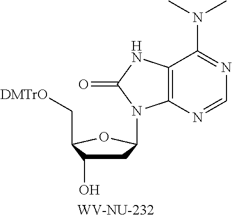

Step 1. To a solution of 9-((2R,4S,5R)-4-hydroxy-5-(hydroxymethyl)tetrahydrofuran-2-yl)-1,9-dihydro-6H-purin-6-one (45 g, 178.41 mmol) in DMF (500 mL) was added BOP (157.82 g, 356.82 mmol) and TEA (36.11 g, 356.82 mmol). The mixture was stirred at 15° C. for 1 hr. The mixture was concentrated under reduced pressure to give a residue. The mixture was without further purification. (2R,3S,5R)-5-(6-((1H-benzo[d][1,2,3]triazol-1-yl)oxy)-9H-purin-9-yl)-2-(hydroxymethyl)tetrahydrofuran-3-ol (65.89 g crude) was obtained as a white solid. TLC (Dichloromethane: Methanol=6:1), Rf=0.48.

Step 2. To a solution of (2R,3S,5R)-5-(6-((1H-benzo[d][1,2,3]triazol-1-yl)oxy)-9H-purin-9-yl)-2-(hydroxymethyl)tetrahydrofuran-3-ol (64.65 g, 174.09 mmol) in DMF (650 mL) was added Cs₂CO₃ (170.17 g, 522.28 mmol) and N-methylmethanamine (23.54 g, 208.91 mmol, 40% purity,). The mixture was stirred at 15° C. for 3 hr. The reaction was clean according to TLC. The mixture was filtered and concentrated in vacuum. The residue was purified by column chromatography (SiO₂, Dichloromethane: Methanol=100/1 to 0/1). (2R,3S,5R)-5-(6-(dimethylamino)-9H-purin-9-yl)-2-(hydroxymethyl)tetrahydrofuran-3-ol (30 g, 61.70% yield) was obtained as a white solid. TLC (Dichloromethane: Methanol=5:1), Rf=0.55.

Step 3. To a solution of (2R,3S,5R)-5-(6-(dimethylamino)-9H-purin-9-yl)-2-(hydroxymethyl)tetrahydrofuran-3-ol (6.5 g, 23.27 mmol) in sodium; acetate (0.5 M, 260.00 mL, 5.59 eq) and AcOH (0.5 M) buffer (pH 4.7), was added Br₂ (4.46 g, 27.93 mmol, 1.44 mL, 1.2 eq). The mixture was stirred at 15° C. for 12 hr. Filtered and concentrated under reduced pressure to give a residue. The residue was purified by column chromatography (SiO₂, DCM: MeOH=100/1 to 0/1). (2R,3S,5R)-5-(8-bromo-6-(dimethylamino)-9H-purin-9-yl)-2-(hydroxymethyl)tetrahydrofuran-3-ol (11 g, 65.98% yield) was obtained as a yellow solid. H NMR (DMSO-d6, 400 MHz): δ=8.10-8.30 (m, 1H), 6.32 (t, J=7.2 Hz, 1H), 4.40-4.61 (m, 1H), 3.82-3.99 (m, 1H), 3.67 (br dd, J=11.9, 4.5 Hz, 3H), 3.49 (br dd, J=11.8, 4.8 Hz, 6H), 3.23 (ddd, J=13.4, 7.5, 6.3 Hz, 2H), 2.19 ppm (ddd, J=13.2, 6.5, 2.5 Hz, 1H); TLC (Ethyl acetate:Dichloromethane=10:1) Rf=0.43.

Step 4. To a solution of Na (4.49 g, 195.43 mmol) in BnOH (200 mL) the mixture was stirred at 15° C. for 1 hr, and then (2R,3S,5R)-5-(8-bromo-6-(dimethylamino)-9H-purin-9-yl)-2-(hydroxymethyl)tetrahydrofuran-3-ol (7 g, 19.54 mmol) in BnOH (100 mL) was added dropwise at 15° C. The resulting mixture was stirred at 60° C. for 2 hr. The reaction mixture was quenched by addition EtOH (10 mL). The residue was purified by column chromatography (SiO₂, Dichloromethane=100/0 to 0/1). (2R,3S,5R)-5-(8-(benzyloxy)-6-(dimethylamino)-9H-purin-9-yl)-2-(hydroxymethyl)tetrahydrofuran-3-ol (7 g, 30.98% yield) as a yellow oil was obtained. ¹H NMR (DMSO-d6, 400 MHz): δ=7.50-7.60 (m, 2H), 7.36-7.48 (m, 3H), 6.24 (dd, J=7.9, 6.8 Hz, 1H), 5.48-5.58 (m, 2H), 5.13-5.28 (m, 2H), 4.31-4.44 (m, 1H), 3.76-3.85 (m, 1H), 3.56 (dt, J=11.8, 4.8 Hz, 1H), 3.37-3.43 (m, 6H), 2.89-3.07 (m, 1H), 2.10 ppm (ddd, J=13.1, 6.5, 2.7 Hz, 1H); TLC (Ethyl acetate:Dichloromethane=10:1, Rf=0.2).

Step 5. To a solution of (2R,3S,5R)-5-(8-(benzyloxy)-6-(dimethylamino)-9H-purin-9-yl)-2-(hydroxymethyl)tetrahydrofuran-3-ol (7 g, 18.16 mmol) in MeOH (200 mL) was added Pd/C (1 g, 18.16 mmol, 10% purity) under N₂ atmosphere. The suspension was degassed and purged with H₂ for 3 times. The mixture was stirred under H₂ (15 Psi) at 15° C. for 12 hr. The mixture was filtered and concentrated under reduced pressure to give a residue. The residue was purified by column chromatography (SiO₂, DCM: MeOH=100/1 to 5/1). 6-(dimethylamino)-9-((2R,4S,5R)-4-hydroxy-5-(hydroxymethyl)tetrahydrofuran-2-yl)-7,9-dihydro-8H-purin-8-one (5.3 g, 98.82% yield) was obtained as a white solid. LCMS (M+H⁺)=296; TLC (DCM: MeOH=10:1), Rf=0.2.

Step 6. To a solution of 6-(dimethylamino)-9-((2R,4S,5R)-4-hydroxy-5-(hydroxymethyl)tetrahydrofuran-2-yl)-7,9-dihydro-8H-purin-8-one (5 g, 16.93 mmol) in Py (100 mL) was added DMTCl (5.74 g, 16.93 mmol). The mixture was stirred at 15° C. for 2 hr. The reaction was clean according to TLC. The reaction mixture was partitioned between H₂O (200 mL) and EtOAc (300 mL). The organic phase was separated, washed with brine 100 mL, dried over Na₂SO₄, filtered and concentrated under reduced pressure to give a residue. The residue was purified by column chromatography (SiO₂, Dichloromethane: Methanol=100/1 to 0/1). Compound WV-NU-232 (7 g, 66.06% yield) as a white solid was obtained. ¹H NMR (DMSO-d6, 400 MHz): δ=10.90 (s, 1H), 7.96 (s, 1H), 7.36 (d, J=7.3 Hz, 2H), 7.15-7.30 (m, 7H), 6.80 (dd, J=19.9, 8.8 Hz, 4H), 6.21 (t, J=6.7 Hz, 1H), 5.22 (br d, J=4.6 Hz. 1H), 4.46-4.54 (m, 1H), 3.88-3.96 (m, 1H), 3.72 (d, J=5.8 Hz, 6H), 3.10-3.22 (m, 8H), 3.05 (dt, J=12.9, 6.4 Hz, 1H), 2.09 ppm (ddd, J=12.9, 7.7, 4.9 Hz, 1H); LCMS (M−H+)=596, purity=95.5%; TLC (Dichloromethane: Methanol =10:1), Rf=0.6.

### Synthesis of 1-((2R,4S,5R)-5-((bis(4-methoxyphenyl)(phenyl)methoxy)methyl)-4-hydroxytetrahydrofuran-2-yl)-5-(prop-1-yn-1-yl)pyrimidine-2,4(1H,3H)-dione (WV-292)

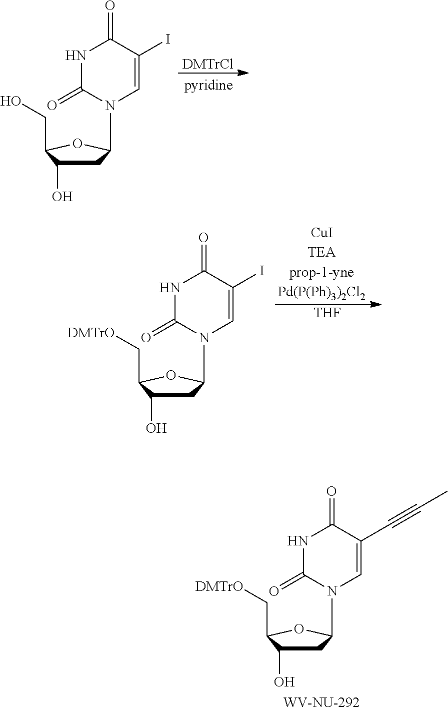

Step 1. To a solution of 1-((2R,4S,5R)-4-hydroxy-5-(hydroxymethyl)tetrahydrofuran-2-yl)-5-iodopyrimidine-2,4(1H,3H)-dione (20 g, 56.48 mmol) in Pyridine (1000 mL) was added DMTCl (19.14 g, 56.48 mmol) and DMAP (7.59 g, 62.13 mmol). The mixture was stirred at 25° C. for 2 hr. The reaction mixture was quenched by addition sat.Na₂CO₃ 200 mL, and then diluted with water 300 mL and extracted with EtOAc mL (200 mL*3). The combined organic layers were washed with brine 200 mL, dried over MgSO₄, filtered and concentrated under reduced pressure to give a residue. The residue was purified by column chromatography (SiO₂, Petroleum ether/Ethyl acetate=1/0 to 0/1). 1-((2R,4S,5R)-5-((bis(4-methoxyphenyl)(phenyl)methoxy)methyl)-4-hydroxytetrahydrofuran-2-yl)-5-iodopyrimidine-2,4(1H,3H)-dione (16 g, 43.15% yield) was obtained as a white solid. ¹HNMR (400 MHz, DMSO-d₆) 6=11.74 (br s, 1H), 8.01 (s, 1H), 7.42-7.37 (m, 2H), 7.36-7.26 (m, 6H), 7.23 (d, J=7.1 Hz, 1H), 6.90 (d, J=8.8 Hz, 4H), 6.10 (t, J=6.8 Hz, 1H), 5.30 (d, J=4.3 Hz, 1H), 4.22 (br d, J=2.0 Hz, 1H), 3.90 (q, J=3.8 Hz, 1H), 3.74 (s, 6H), 3.23-3.12 (m, 2H), 2.31-2.12 (m, 2H); LCMS (M−H+): 655.3; TLC (Petroleum ether:Ethyl acetate=1:1), Rf=0.35.

Step 2. To a solution of 1-((2R,4S,5R)-5-((bis(4-methoxyphenyl)(phenyl)methoxy)methyl)-4-hydroxytetrahydrofuran-2-yl)-5-iodopyrimidine-2,4(1H,3H)-dione (11.87 g, 18.08 mmol) in THF (1000 mL) was added iodocopper (1.72 g, 9.04 mmol) and dichloropalladium; triphenylphosphane (1.27 g, 1.81 mmol) and TEA (7.32 g, 72.33 mmol), prop-1-yne (1 M, 180.82 mL) in N₂. The mixture was stirred at 25° C. for 5 hr. LCMS showed the product was detected. The reaction mixture was quenched by addition sat.Na₂CO₃ 200 mL, and then diluted with water 300 mL and extracted with EtOAc mL (200 mL*3). The combined organic layers were washed with brine 200 mL, dried over MgSO₄, filtered and concentrated under reduced pressure to give a residue. The residue was purified by column chromatography (SiO₂, Petroleum ether/Ethyl acetate =1/0 to 0/1). Compound WV-NU-292 (8.58 g, 63.87% yield) was obtained as a yellow solid. ¹H NMR (400 MHz, DMSO-d6) δ=11.65-11.56 (m, 1H), 7.93-7.82 (m, 1H), 7.41 (d, J=7.6 Hz, 2H), 7.35-7.27 (m, 6H), 7.26-7.20 (m, 1H), 6.89 (d, J=8.5 Hz, 4H), 6.13 (t, J=6.8 Hz, 1H), 5.33 (d, J=4.4 Hz, 1H), 4.35-4.24 (m, 1H), 3.96-3.88 (m, 1H), 3.74 (s, 6H), 3.30-3.22 (m, 1H), 3.10 (dd, J=2.4, 10.4 Hz, 1H), 2.33-2.14 (m, 2H), 1.79 (s, 3H); LCMS (M−H+): 567.3, LCMS purity: 91.89%; TLC (Petroleum ether:Ethyl acetate)=1:1, Rf=0.2.

### Synthesis of 1-((2R,3R,4R,5R)-5-((bis(4-methoxyphenyl)(phenyl)methoxy)methyl)-3-fluoro-4-hydroxytetrahydrofuran-2-yl)-5-(prop-1-yn-1-yl)pyrimidine-2,4(1H,3H)-dione (WV-293)

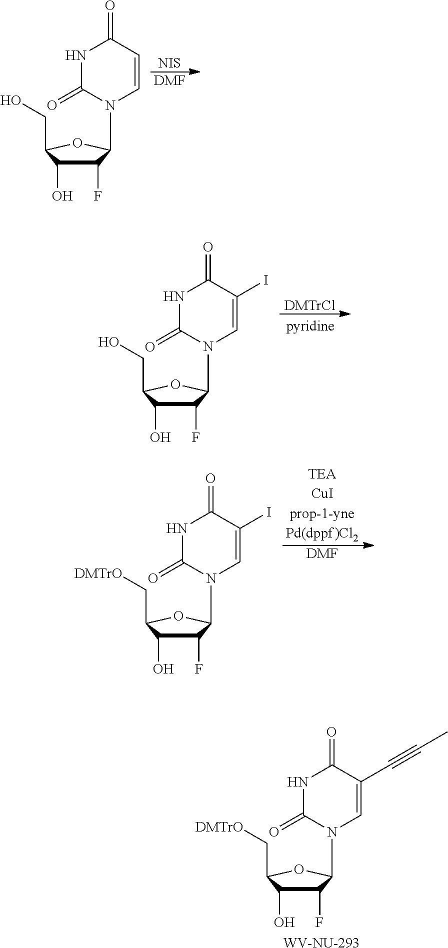

Step 1. To a solution of 1-((2R,3R,4R,5R)-3-fluoro-4-hydroxy-5-(hydroxymethyl)tetrahydrofuran-2-yl)pyrimidine-2,4(1H,3H)-dione (24 g, 97.48 mmol) in DMF (800 mL) was added NIS (65.80 g, 292.45 mmol). The mixture was stirred at 90° C. for 12 hr. The reaction was clean according to TLC. The reaction mixture was concentrated under reduced pressure to remove DMF. The residue was purified by column chromatography (SiO₂, Petroleum ether/Ethyl acetate=I/O to 0/1). 1-((2R,3R,4R,5R)-3-fluoro-4-hydroxy-5-(hydroxymethyl)tetrahydrofuran-2-yl)-5-iodopyrimidine-2,4(1H,3H)-dione (14 g, 38.60% yield) was obtained as a yellow solid. ¹HNMR (400 MHz, DMSO-d₆) δ=1H NMR (400 MHz, DMSO-d6) δ=11.72 (s, 1H), 8.55-8.50 (m, 1H), 5.85 (dd, J=0.9, 16.8 Hz, 1H), 5.59 (br s, 1H), 5.38 (br s, 1H), 5.11-4.93 (m, 1H), 4.23-4.09 (m, 1H), 3.93-3.76 (m, 2H), 3.65-3.54 (m, 1H); LCMS (M−H+): 371.1. TLC (Dichloromethane: Methanol=10:1), Rf=0.4.

Step 2. To a solution of 1-((2R,3R,4R,5R)-3-fluoro-4-hydroxy-5-(hydroxymethyl)tetrahydrofuran-2-yl)-5-iodopyrimidine-2,4(1H,3H)-dione (12.6 g, 33.86 mmol) in PYRIDINE (250 mL) was added DMTCl (12.62 g, 37.25 mmol). The mixture was stirred at 20° C. for 12 hr. The reaction mixture was concentrated under reduced pressure to remove pyridine. The residue was purified by column chromatography (SiO₂, Petroleum ether/Ethyl acetate=1/0 to 0/1). 1-((2R,3R,4R,5R)-5-((bis(4-methoxyphenyl)(phenyl)methoxy)methyl)-3-fluoro-4-hydroxytetrahydrofuran-2-yl)-5-iodopyrimidine-2,4(1H,3H)-dione (20 g, 87.57% yield) was obtained as a white solid. ¹H NMR (400 MHz, DMSO-d6) 6=11.81 (br s, 1H), 8.07 (s, 1H), 7.41 (d, J=7.5 Hz, 2H), 7.34-7.26 (m, 6H), 7.25-7.19 (m, 1H), 6.88 (dd, J=1.8, 8.9 Hz, 4H), 5.87-5.78 (m, 1H), 5.60 (d, J=7.1 Hz, 1H), 5.26-5.06 (m, 1H), 4.39-4.24 (m, 1H), 4.00-3.97 (m, 1H), 3.74 (s, 6H), 3.23 (br d, J=3.1 Hz, 2H); LCMS (M−H⁺): 653.2; TLC (Petroleum ether:Ethyl acetate=1:1), Rf=0.3.

Step 3. To a solution of 1-((2R,3R,4R,5R)-5-((bis(4-methoxyphenyl)(phenyl)methoxy)methyl)-3-fluoro-4-hydroxytetrahydrofuran-2-yl)-5-iodopyrimidine-2,4(1H,3H)-dione (12 g, 17.79 mmol) in THF (300 mL) was added CuI (1.69 g, 8.90 mmol), Pd(dppf)Cl₂ (2.60 g, 3.56 mmol), TEA (18.00 g, 177.92 mmol) and prop-1-yne (1 M, 177.92 mL) at 25° C. for 12 hr. The reaction was clean according to TLC. The reaction mixture was diluted with Na₂CO₃ 200 mL and extracted with Ethyl acetate (200 mL*3). The combined organic layers were washed with NaCl 500 mL, dried over Na₂SO₄, filtered and concentrated under reduced pressure to give a residue. The residue was purified by prep-HPLC (neutral condition). Compound WV-NU-293 (2.26 g, 23.95% yield) was obtained as a brown solid. ¹HNMR (400 MHz, DMSO-d6) δ=11.68 (br s, 1H), 7.89-7.83 (m, 1H), 7.42 (br d, J=7.6 Hz, 2H), 7.34-7.26 (m, 6H), 7.25-7.19 (m, 1H), 6.88 (br d, J=8.7 Hz, 4H), 5.83 (br d, J=19.3 Hz, 1H), 5.67 (d, J=7.0 Hz, 1H), 5.23-5.04 (m, 1H), 4.46-4.27 (m, 1H), 3.74 (s, 6H), 3.34 (s, 2H), 3.21 (br d, J=10.1 Hz, 1H), 1.74 (s, 3H); LCMS (M−H+): 585.3, LCMS purity: 99.04%; TLC (Petroleum ether:Ethyl acetate)=1:1, Rf=0.2.

### Synthesis of N-(1-((2R,3S,4R,5R)-5-((bis(4-methoxyphenyl)(phenyl)methoxy)methyl)-3-fluoro-4-hydroxytetrahydrofuran-2-yl)-2-oxo-1,2-dihydropyrimidin-4-yl)acetamide (WV-285)

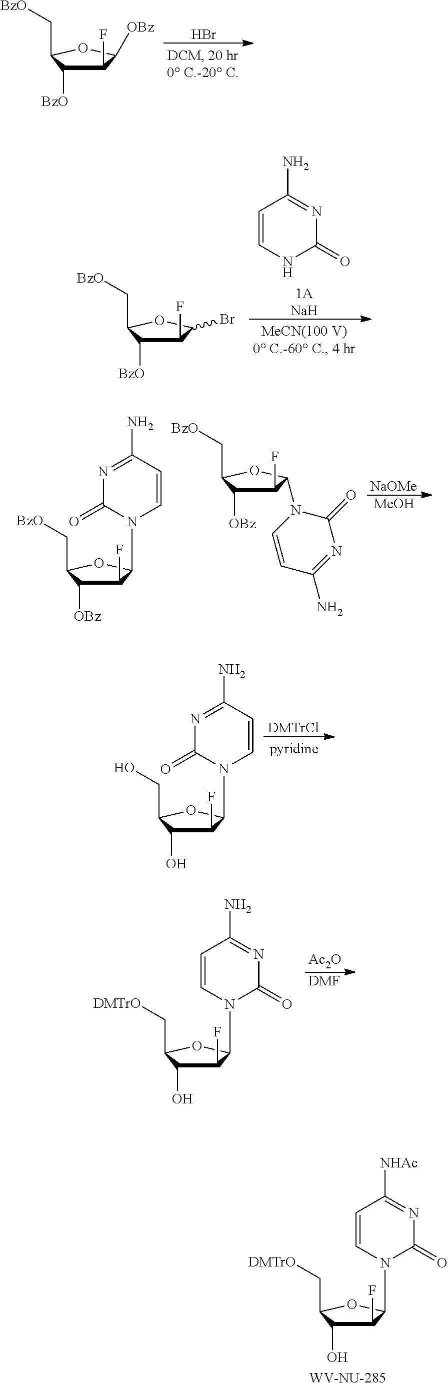

Step 1. To a solution of (2S,3S,4R,5R)-5-((benzoyloxy)methyl)-3-fluorotetrahydrofuran-2,4-diyl dibenzoate (50 g, 107.66 mmol) in DCM (500 mL) was added hydrogen bromide (105.58 g, 430.63 mmol) at 0° C. in N₂. The mixture was stirred at 0-20° C. for 20 hr. The reaction mixture of two batches poured into 500 mL of ice water, and the organic phase was separated, washed with water (5×200 mL), dried over MgSO₄, and evaporated under reduced pressure. And then the crude product to remove remaining acetic acid and dried in vacuum. ((2R,3R,4S)-3-(benzoyloxy)-5-bromo-4-fluorotetrahydrofuran-2-yl)methyl benzoate (45 g, crude) was obtained as a yellow oil. TLC: Petroleum ether:Ethyl acetate=3:1, Rf=0.37.

Step 2. To a solution of 4-aminopyrimidin-2(1H)-one (17.72 g, 159.49 mmol) in ACN (2200 mL) was added NaH (8.51 g, 212.65 mmol) at 0° C. for 0.5 hr. And then added ((2R,3R,4S)-3-(benzoyloxy)-5-bromo-4-fluorotetrahydrofuran-2-yl)methyl benzoate (45 g, 106.33 mmol). The mixture was stirred at 0-60° C. for 3 hr. The reaction mixture was filtered and concentrated under reduced pressure to give a residue. The residue was purified by column chromatography (SiO₂, Petroleum ether:Ethyl acetate=10:1 to 0:1), and got 23 g the mixture of product, the mixture of product purification by prep-HPLC: (column: Welch Xtimate C18 250*70 mm #10 um; mobile phase: [water (NH₄HCO₃) -ACN]; B %: 35%-65%, 20 min). (2R,3R,4S,5R)-5-(4-amino-2-oxopyrimidin-1(2H)-yl)-2-((benzoyloxy)methyl)-4-fluorotetrahydrofuran-3-yl benzoate (5.7 g, 12.57 mmol) was obtained as a yellow solid. ¹H NMR (400 MHz, DMSO-d6) δ=8.10-7.95 (m, 4H), 7.77-7.63 (m, 2H), 7.61-7.50 (m, 5H), 7.34 (br s, 1H), 7.29 (br s, 1H), 6.36-6.26 (m, 1H), 5.76-5.60 (m, 2H), 5.58-5.50 (m, IH), 5.55-5.40 (m, 1H), 4.77-4.56 (m, 3H); LCMS (M+H⁺): 454.2; TLC: Petroleum ether: Ethyl acetate=1:1.5, Rf=0.04.

Step 3. To a solution of (2R,3R,4S,5R)-5-(4-amino-2-oxopyrimidin-1(2H)-yl)-2-((benzoyloxy)methyl)-4-fluorotetrahydrofuran-3-yl benzoate (5.6 g, 12.35 mmol) in MeOH (100 mL) was added NaOMe (1.47 g, 27.17 mmol) in N₂. The mixture was stirred at 25° C. for 3 hr. The reaction was added NH₄Cl (12 g) and stirred at room temperature for 5 min, and then vacuum concentration. The residue was purified by column chromatography (SiO₂, Petroleum ether:Ethyl acetate=10:1 to 0:1 to Ethyl acetate: MeOH=2:1). 4-amino-1-((2R,3S,4R,5R)-3-fluoro-4-hydroxy-5-(hydroxymethyl)tetrahydrofuran-2-yl)pyrimidin-2(1H)-one (3 g, 99.06% yield) was obtained as a white solid. ¹H NMR (400 MHz, DMSO-d6) δ=7.64 (dd, J=1.2, 7.4 Hz, 1H), 7.36 (br s, 1H), 7.21 (br s, 1H), 6.08 (dd, J=3.7, 18.6 Hz, 1H), 5.88 (br d, J=4.5 Hz, 1H), 5.76 (d, J=7.5 Hz, 1H), 5.06 (br s, 1H), 5.03-4.86 (m, 1H), 4.17 (br dd, J=1.7, 18.8 Hz, 1H), 3.89-3.71 (m, 1H), 3.66-3.50 (m, 2H); LCMS (M+H⁺): 246.1, purity: 100%; TLC: Petroleum ether:Ethyl acetate=0:1, Rf=0.00.

Step 4. To a solution of 4-amino-1-((2R,3S,4R,5R)-3-fluoro-4-hydroxy-5-(hydroxymethyl)tetrahydrofuran-2-yl)pyrimidin-2(1H)-one (3 g, 12.23 mmol) in Py (10 mL) was added DMT-Cl (4.15 g, 12.23 mmol) in N₂. The mixture was stirred at 25° C. for 6 hr. The reaction mixture was filtered and concentrated under reduced pressure to give a residue. The residue was purified by column chromatography (SiO₂, Petroleum ether:Ethyl acetate=5: 1 to 0: 1 to Ethyl acetate: MeOH=1:1). 4-amino-1-((2R,3S,4R,5R)-5-((bis(4-methoxyphenyl)(phenyl)methoxy)methyl)-3-fluoro-4-hydroxytetrahydrofuran-2-yl)pyrimidin-2(1H)-one (2.7 g, 40.30% yield) was obtained as a yellow solid. LCMS (M−H⁺): 546.4, purity: 92.76%; TLC:Ethyl acetate:Methanol=8:1, Rf=0.34.

Step 5. To a solution of 4-amino-1-((2R,3S,4R,5R)-5-((bis(4-methoxyphenyl)(phenyl)methoxy)methyl)-3-fluoro-4-hydroxytetrahydrofuran-2-yl)pyrimidin-2(1H)-one (2.7 g, 4.93 mmol) in DMF (30 mL) was added Ac₂O (1.11 g, 10.85 mmol) in N₂. The mixture was stirred at 25° C. for 2 hr. The reaction mixture was filtered and concentrated under reduced pressure to give a residue. The residue was purified by column chromatography (SiO₂, Dichloromethane: Methanol=1:0 to 10:1, 5% TEA). Compound WV-NU-285 (2.3 g, 79.11% yield) was obtained as a white solid. ¹H NMR (400 MHz, DMSO-d6) δ=10.95 (s, 1H), 7.91 (d, J=7.5 Hz, 1H), 7.42-7.22 (m, 9H), 7.14 (d, J=7.5 Hz, 1H), 6.91 (d, J=8.5 Hz, 4H), 6.17 (dd, J=3.9, 16.1 Hz, 1H), 6.00 (d, J=4.9 Hz, 1H), 5.19-5.00 (m, 1H), 4.29-4.20 (m, 1H), 4.10-4.06 (m, 1H), 3.74 (s, 6H), 3.30-3.26 (m, 2H), 2.10 (s. 3H); LCMS (M−H+):588.3, purity: 98.75%; TLC: Dichloromethane: Methanol=10:1, Rf=0.38.

### Synthesis of 1-(9-((2R,4S,5R)-5-((bis(4-methoxyphenyl)(phenyl)methoxy)methyl)-4-hydroxytetrahydrofuran-2-yl)-8-oxo-8,9-dihydro-7H-purin-6-yl)-3-methylurea (WV-247)

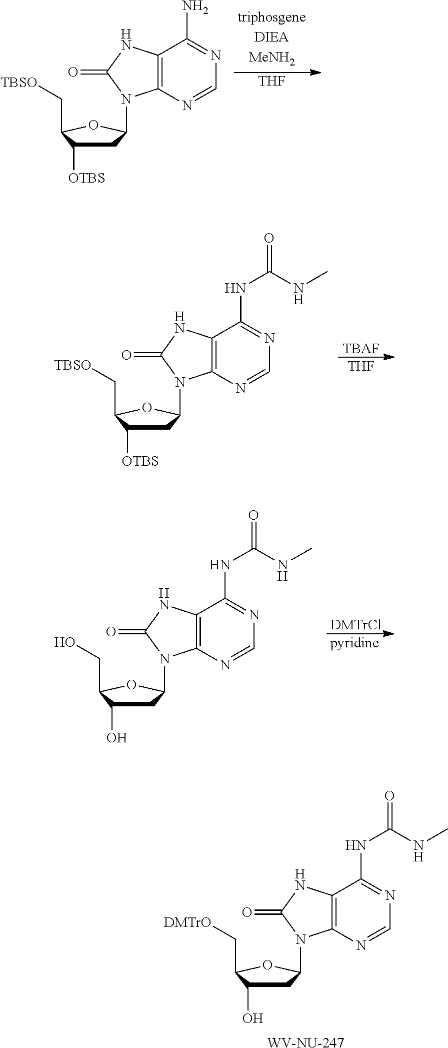

Step 1. For three batches: To a solution of 6-amino-9-((2R,4S,5R)-4-((tert-butyldimethylsilyl)oxy)-5-(((tert-butyldimethylsilyl)oxy)methyl)tetrahydrofuran-2-yl)-7,9-dihydro-8H-purin-8-one (10 g, 20.17 mmol), DIEA (6.52 g, 50.43 mmol) in THF (100 mL) and the triphosgene (2.68 g, 9.03 mmol) was added at 0° C., the mixture was stirred at 0° C. for 30 min and then MeNH₂ (37.7 g, 364.13 mmol, 30% purity) was added and the mixture was stirred at 15° C. for 12 hr. The mixture was poured into ice water (200 mL) and extracted with EtOAc (200 mL*2), the combined organic was dried over Na₂SO₄, filtered and concentrated to get the crude. The residue was purified by MPLC (Petroleum ether:Ethyl acetate=10:1, 5:1) and then the mixture was triturated with by (Petroleum ether:Ethyl acetate=10:1), filtered and the cake was concentrated to get 1-(9-((2R,4S,5R)-4-((tert-butyldimethylsilyl)oxy)-5-(((tert-butyldimethylsilyl)oxy)methyl)tetrahydrofuran-2-yl)-8-oxo-8,9-dihydro-7H-purin-6-yl)-3-methylurea (6 g, 17.93% yield) and 6-amino-9-((2R,4S,5R)-4-((tert-butyldimethylsilyl)oxy)-5-(((tert-butyldimethylsilyl)oxy)methyl)tetrahydrofuran-2-yl)-7,9-dihydro-8H-purin-8-one (7 g, 23.33% yield) as a yellow solid recovered. ¹HNMR (400 MHz, DMSO-d6) δ=10.22-10.08 (m, 1H), 9.32-9.11 (m, 1H), 8.21-8.18 (m, 1H), 8.17-8.10 (m, 1H), 6.08-6.00 (m, 1H), 4.65-4.56 (m, 1H), 3.70-3.60 (m, 2H), 3.51-3.46 (m, 1H), 3.14-3.01 (m, 1H), 2.70-2.64 (m, 3H), 2.04-1.95 (m, 1H), 0.82-0.78 (m, 9H), 0.73-0.70 (m, 9H), 0.00 (s, 6H), −0.10-−0.18 (m, 6H); LCMS (M+H⁺): 553.2; TLC: Petroleum ether:Ethyl acetate=3:1, Rf =0.61.

Step 2. To a solution of 1-(9-((2R,4S,5R)-4-((tert-butyldimethylsilyl)oxy)-5-(((tert-butyldimethylsilyl)oxy)methyl)tetrahydrofuran-2-yl)-8-oxo-8,9-dihydro-7H-purin-6-yl)-3-methylurea (6 g, 10.85 mmol, 1 eq) in THF (80 mL) was added TBAF (1 M, 27.13 mL, 2.5 eq). The mixture was stirred at 20° C. for 1 hr. The reaction mixture was concentrated under reduced pressure to remove solvent. The crude product was purified by MPLC (SiO₂, Dichloromethane: Methanol=5:1, 10:1, 8:1) to get 1-(9-((2R,4S,5R)-4-hydroxy-5-(hydroxymethyl)tetrahydrofuran-2-yl)-8-oxo-8,9-dihydro-7H-purin-6-yl)-3-methylurea (3.5 g crude) as a yellow oil. LCMS (M+H⁺): 325.1; TLC (Dichloromethane: Methanol=8:1), Rf=0.15.

Step 3. To a solution of 1-(9-((2R,4S,5R)-4-hydroxy-5-(hydroxymethyl)tetrahydrofuran-2-yl)-8-oxo-8,9-dihydro-7H-purin-6-yl)-3-methylurea (3.5 g, 10.79 mmol) in PYRIDINE (50 mL) and DMTCl (3.66 g, 10.79 mmol) was added, the mixture was stirred at 25° C. for 1 hr. MeOH (5 mL) was added and stirred for 5 min and the mixture was concentrated to get the crude. The residue was purified by column chromatography (SiO₂, Petroleum ether/Ethyl acetate=1/0 to 0/1, then Ethyl acetate/methanol=1/0 to 0/1, 5% TEA) to get WV-NU-247 (4 g, 58.87% yield) was obtained as a yellow solid ¹H NMR (400 MHz, DMSO-d₆) δ=10.34-10.20 (m, 1H), 9.39-9.22 (m, 1H), 8.30-8.23 (m, 1H), 8.16-8.13 (m, 1H), 7.34 (d, J=7.4 Hz, 2H), 7.25-7.16 (m, 7H), 6.85-6.72 (m, 4H), 6.22-6.14 (m, 1H), 5.29-5.18 (m, 1H), 4.53-4.42 (m, 1H), 3.96-3.89 (m, 1H), 3.70 (d, J=6.9 Hz, 6H), 3.21-3.09 (m, 2H), 3.06-2.97 (m, 1H), 2.81-2.75 (m, 3H), 2.17-2.04 (m, 1H); LCMS (M−H⁺): 625.3; purity: 98.6% purity; TLC (Dichloromethane: Methanol=10:1), Rf=0.43.

### Synthesis of (2R,3S,5R)-2-((bis(4-methoxyphenyl)(phenyl)methoxy)methyl)-5-(9H-purin-9-yl)tetrahydrofuran-3-ol (WV-298)

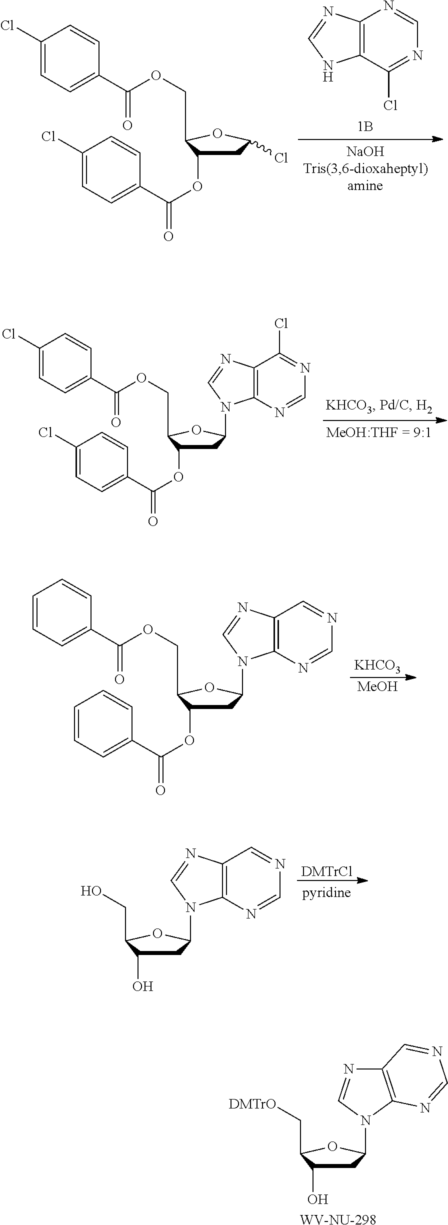

Step 1. To a solution of 6-chloro-7H-purine (10 g, 64.70 mmol) in ACN (1000 mL) was added NaOH (18.00 g, 450.03 mmol), 2-(2-methoxyethoxy)-N, N-bis [2-(2-methoxyethoxy) ethyl]ethanamine (209.26 mg, 647.01 umol) and (2R,3S)-5-chloro-2-(((4-chlorobenzoyl)oxy)methyl)tetrahydrofuran-3-yl 4-chlorobenzoate (30.58 g, 71.17 mmol). The mixture was stirred at 25° C. for 12 hr. The reaction mixture was filtered and concentrated under reduced pressure to give a residue. The residue was purified by column chromatography (SiO2, Petroleum ether/Ethyl acetate=1/0 to 0/1). (2R,3S,5R)-5-(6-chloro-9H-purin-9-yl)-2-(((4-chlorobenzoyl)oxy)methyl)tetrahydrofuran-3-yl 4-chlorobenzoate (29 g, 81.83% yield) was obtained as a yellow solid. ¹HNMR (400 MHz, DMSO-d₆) 6=9.15 (s, 1H), 8.84 (s, 1H), 8.04 (d, J=8.6 Hz, 2H), 7.85 (d, J=8.6 Hz, 2H), 7.67-7.50 (m, 4H), 6.88 (t, J=6.5 Hz, 1H), 5.79-5.73 (m, IH), 4.73-4.68 (m, 1H), 4.65-4.54 (m, 2H), 3.25-3.15 (m, 1H), 2.98 (ddd, J=3.5, 6.2, 14.5 Hz, 1H); LCMS (M−H+): 569; TLC (Petroleum ether:Ethyl acetate=1:1), Rf=0.6.

Step 2. For six batches: To a solution of (2R,3S,5R)-5-(6-chloro-9H-purin-9-yl)-2-(((4-chlorobenzoyl)oxy)methyl)tetrahydrofuran-3-yl 4-chlorobenzoate (25 g, 45.64 mmol) in MeOH (450 mL) and THF (50 mL) was added Pd/C (3 g, 45.64 mmol, 30% purity) and KHCO₃ (10.05 g, 100.41 mmol). The mixture was stirred at 25° C. for 15 hr under H₂ (15 psi). The reaction mixture was filtered and concentrated under reduced pressure to give a residue. For batches were combined for workup. ((2R,3S,5R)-3-(benzoyloxy)-5-(9H-purin-9-yl)tetrahydrofuran-2-yl)methyl benzoate (80 g, crude) was obtained as a yellow solid. LCMS (M−H⁺): 445.3.

Step 3. For three batches. To a solution of ((2R,3S,5R)-3-(benzoyloxy)-5-(9H-purin-9-yl)tetrahydrofuran-2-yl)methyl benzoate (40 g, 90.00 mmol) in MeOH (500 mL) was added KHCO₃ (18.02 g, 180.00 mmol). The mixture was stirred at 25° C. for 1 hr. TLC indicated compound 3 was consumed completely and one new spot formed. The reaction was clean according to TLC. The reaction mixture was concentrated under reduced pressure to give a residue. For batches were combined for workup. The crude product was purified by re- crystallization from Petroleum ether (500 ml): Ethyl acetate (100 mL) at 25° C. (2R,3S,5R)-2-(hydroxymethyl)-5-(9H-purin-9-yl)tetrahydrofuran-3-ol (60 g, 4.07% yield) was obtained as a white solid. ¹HNMR (400 MHz, DMSO-d₆) 6=9.18 (s, 1H), 8.95 (s, 1H), 8.82 (s, 1H), 6.49 (br t, J=6.4 Hz, 1H), 4.45 (br s, 1H), 3.90 (br s, 1H), 3.68-3.49 (m, 2H), 2.79 (td, J=6.3, 12.7 Hz, 1H), 2.42-2.32 (m, 1H); LCMS (M−H+): 237.2; TLC (Petroleum ether:Ethyl acetate)=1:1, Rf=0.3.

Step 4. For three batches. To a solution of (2R,3S,5R)-2-(hydroxymethyl)-5-(9H-purin-9-yl)tetrahydrofuran-3-ol (20 g, 84.66 mmol) in Py (700 mL) added DMTCl (28.69 g, 84.66 mmol). The mixture was stirred at 25° C. for 15 hr. The reaction mixture was added Methanol (50 ml) and concentrated under reduced pressure to give a residue. For batches were combined for workup. The residue was purified by column chromatography (SiO₂, Petroleum ether/Ethyl acetate=I/O to 0/1 to Dichloromethane: Methanol=1/0 to 0/1). Compound WV-NU-298 (30 g, 22.06% yield) was obtained as an orange solid. ¹HNMR (400 MHz, DMSO-d₆) δ=9.18 (s, 1H), 8.90-8.81 (m, 1H), 8.77-8.68 (m, 1H), 7.40-7.24 (m, 3H), 7.21-7.10 (m, 7H), 6.82-6.70 (m, 4H), 6.55-6.46 (m, 1H), 5.48-5.37 (m, 1H), 4.61-4.47 (m, 1H), 3.75-3.68 (m, 6H), 3.23-3.14 (m, 2H), 3.03-2.90 (m, 1H), 2.44-2.33 (m, 1H); LCMS (M−H⁺): 537.3, LCMS purity: 90.13%; TLC (Petroleum ether:Ethyl acetate)=0:1, Rf=0.3.

### Synthesis of 1-((S)-3-(bis(4-methoxyphenyl)(phenyl)methoxy)-2-(((1S,3S,3aS)-3-((phenylsulfonyl)methyl)tetrahydro-1H,3H-pyrrolo[1,2-c][1,3,2]oxazaphosphol-1-yl)oxy)propyl)pyrimidine-2,4(1H,3H)-dione

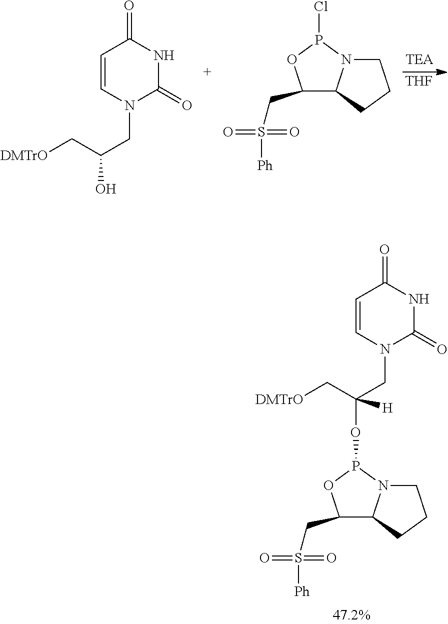

To a solution of dry 1-[(2S)-3-[bis(4-methoxyphenyl)-phenyl-methoxy]-2-hydroxy-propyl]pyrimidine-2,4-dione (787 mg, 1.61 mmol) in THF (6 mL) was added triethylamine (0.56 mL, 4.03 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.75M in THF, 3.87 mL, 2.90 mmol) was added fast dropwise. The ice/water bath was removed. The resulting slurry was stirred at rt for 3 hr. TLC and LCMS showed that the r×n was complete. The reaction was quenched by water (15 L). Anhydrous MgSO₄ (193 mg) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a brownish off-white foam (0.587 g, 47.2% yield). ¹H NMR (600 MHz, Chloroform-d) δ 8.50 (s, 1H), 7.98-7.93 (m, 2H), 7.67-7.63 (m, 1H), 7.58-7.53 (m, 2H), 7.48-7.44 (m, 2H), 7.36-7.32 (m, 4H), 7.31-7.27 (m, 2H), 7.24-7.20 (m, 1H), 7.14 (d, J=7.9 Hz, 1H), 6.86-6.81 (m, 4H), 5.61 (d, J=7.9 Hz, 1H), 5.12 (q, J=6.2 Hz, 1H), 4.44 (tdd, J=10.4, 8.8, 7.3, 4.4 Hz, 1H), 4.08 (dd, J=14.0, 3.5 Hz, 1H), 3.793 (s, 3H), 3.791 (s, 3H), 3.78-3.72 (m, 1H), 3.53-3.44 (m, 3H), 3.32 (dd, J=14.2, 6.9 Hz, 1H), 3.19 (dd, J=10.0, 5.6 Hz, 1H), 3.14 (dd, J=10.0, 4.6 Hz, 1H), 3.09 (tdd, J=10.6, 8.7, 4.2 Hz, 1H), 1.85 (ttd, J=13.9, 9.5, 8.8, 4.6 Hz, 1H), 1.78 (td, J=12.2, 10.5, 6.9 Hz, 1H), 1.69 (dtd, J=12.1, 7.1, 6.6, 2.6 Hz, 1H), 1.13-1.05 (m, 1H); ³¹P NMR (243 MHz, Chloroform-d) δ 155.42; MS (ESI), 770.09 [M−H]⁻.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-5-methylpyrimidine-2,4(1H,3H)-dione

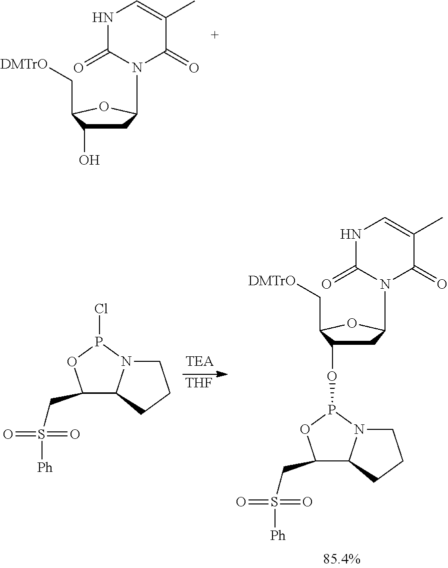

To a solution of dry 3-[(2R,4R,5R)-5-[[bis(4-methoxyphenyl)-phenyl-methoxy]methyl]-4-hydroxy-tetrahydrofuran-2-yl]-5-methyl-1H-pyrimidine-2,4-dione (4.80 g, 8.81 mmol) in THF (36 mL) was added triethylamine (3.07 mL, 22.0 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.75M in THF, 21.2 mL, 15.9 mmol) was added fast dropwise. The ice/water bath was removed. The resulting slurry was stirred at rt for 3 hr 45 min. TLC and LCMS showed that the r×n was complete. The reaction was quenched by water (79 μL). Anhydrous MgSO₄ (1.06 g) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as a white foam (6.23 g, 85.4% yield). ¹H NMR (600 MHz, Chloroform-d) δ 8.89 (s, 1H), 7.88-7.84 (m, 2H), 7.60 (tt, J=7.4, 1.3 Hz, 1H), 7.52-7.46 (m, 4H), 7.38-7.34 (m, 4H), 7.22 (t, J=7.7 Hz, 2H), 7.15 (tt, J=7.3, 1.8 Hz 1H), 6.79-6.75 (m, 4H), 6.74 (dd, J=8.6, 4.8 Hz, 1H), 6.56 (s, 1H), 4.94-4.86 (m, 2H), 3.91 (td, J=6.1, 3.5 Hz, 1H), 3.751 (s, 3H), 3.747 (s, 3H), 3.65-3.57 (m, 1H), 3.43-3.27 (m, 5H), 2.95 (tdd, J=10.3, 8.8, 4.0 Hz, 1H), 2.88 (ddd, J=13.2, 8.2, 4.8 Hz, 1H), 2.25 (ddd, J=13.8, 8.7, 5.6 Hz, 1H), 1.83 (tdd, J=11.5, 5.7, 2.9 Hz, 1H), 1.76 (d, J=1.2 Hz, 3H), 1.70 (dt, J=8.2, 3.9 Hz, 1H), 1.61 (tdt, J=7.8, 5.0, 2.5 Hz, 1H), 1.06 (dtd, J=11.6, 10.1, 8.4 Hz, 1H); ³¹P NMR (243 MHz, Chloroform-d) δ 149.64; MS (ESI), 866.01 [M+K]+.

### Synthesis of 7-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-3,7-dihydro-4H-pyrrolo[2,3-d]pyrimidin-4-one

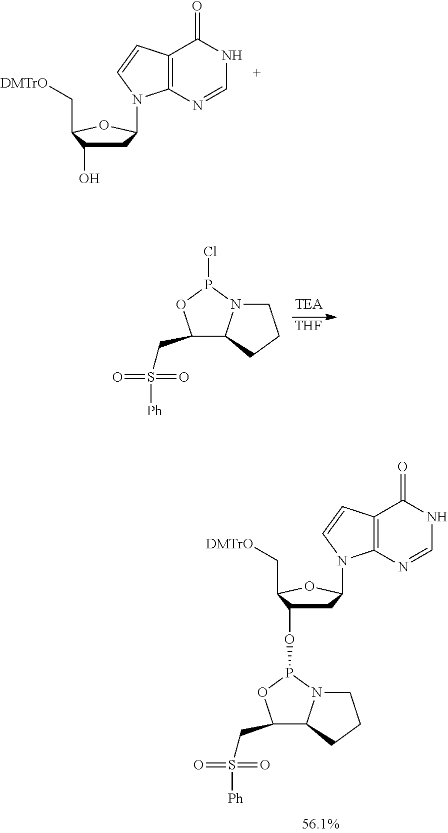

To a solution of dry 7-[(2R,4R,5R)-5-[[bis(4-methoxyphenyl)-phenyl-methoxy]methyl]-4-hydroxy-tetrahydrofuran-2-yl]-3H-pyrrolo[2,3-d]pyrimidin-4-one (3.00 g, 5.42 mmol) in THF (22.5 mL) was added triethylamine (1.89 mL, 13.5 mmol). (3S,3aS)-3-(benzenesulfonylmethyl)-1-chloro-3a,4,5,6-tetrahydro-3H-pyrrolo[1,2-c][1,3,2]oxazaphosphole (0.75M in THF, 13.0 mL, 9.75 mmol) was added fast dropwise. The resulting slurry was stirred at rt for 3 hr. TLC and LCMS showed that the r×n was complete. The reaction was quenched by water (49 μL). Anhydrous MgSO₄ (650 mg) was added. The mixture was filtered through celite, and the filtrate was concentrated. The resulting crude product was purified by normal phase column chromatography applying 0-50% ACN in EtOAc (each mobile phase contained 2.5% triethylamine) as the gradient to afford the title compound as a white foam (2.55 g, 56.1% yield). ¹H NMR (600 MHz, Chloroform-d) δ 12.35 (s, 1H), 7.92 (s, 1H), 7.89 (d, J=7.7 Hz, 2H), 7.58-7.54 (m, 1H), 7.47 (t, J=7.7 Hz, 2H), 7.43 (d, J=7.8 Hz, 2H), 7.34-7.29 (m, 4H), 7.27 (t, J=7.5 Hz, 2H), 7.24-7.19 (m, 1H), 7.12 (d, J=3.6 Hz, 1H), 6.84-6.79 (m, 4H), 6.68 (d, J=3.6 Hz, 1H), 6.60 (dd, J=8.1, 5.8 Hz, 1H), 4.99 (q, J=6.0 Hz, 1H), 4.87 (ddt, J=9.0, 5.9, 2.7 Hz, 1H), 4.08 (q, J=3.7 Hz, 1H), 3.78 (s, 6H), 3.63 (dq, J=11.9, 6.0 Hz, 1H), 3.54-3.43 (m, 2H), 3.40-3.33 (m, 2H), 3.26 (dd, J=10.3, 3.9 Hz, 1H), 3.12 (qd, J=10.0, 4.0 Hz, 1H), 2.61 (ddd, J=13.9, 8.2, 6.2 Hz, 1H), 2.47 (ddd, J=13.6, 5.8, 2.5 Hz, 1H), 1.89 (ddq, J=11.7, 7.6, 3.5, 3.0 Hz, 1H), 1.82-1.74 (m, 1H), 1.65 (qd, J=7.5, 2.5 Hz, 1H), 1.16-1.07 (m, 1H); ³¹P NMR (243 MHz, Chloroform-d) δ 152.69; MS (ESI), 875.10 [M+K]⁺.

### Synthesis of 9-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-6-(isopropylamino)-7,9-dihydro-8H-purin-8-one

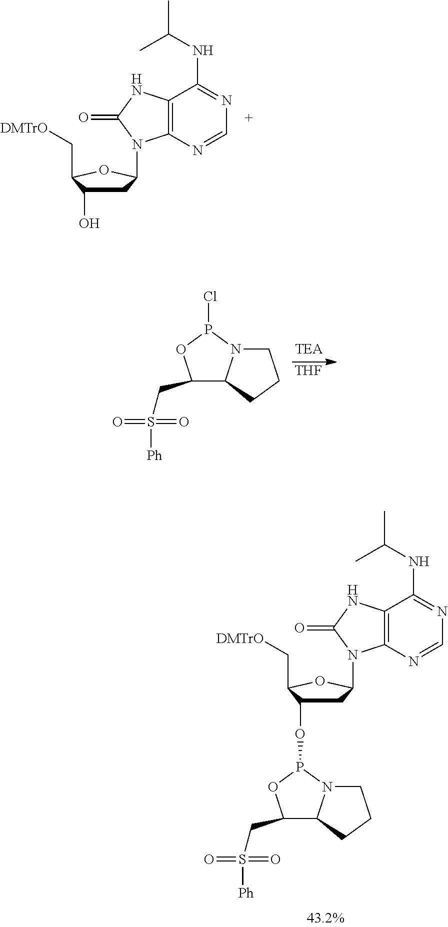

To a solution of dry 9-[(2R,4R,5R)-5-[[bis(4-methoxyphenyl)-phenyl-methoxy]methyl]-4-hydroxy-tetrahydrofuran-2-yl]-6-(isopropylamino)-7H-purin-8-one (2.00 g, 3.27 mmol) in THF (15 mL) was added triethylamine (1.14 mL, 8.17 mmol). (3S,3aS)-3-(benzenesulfonylmethyl)-1-chloro-3a,4,5,6-tetrahydro-3H-pyrrolo[1,2-c][1,3,2]oxazaphosphole (0.75M in THF, 6.98 mL, 5.23 mmol) was added fast dropwise. The resulting slurry was stirred at rt for 3 hr. TLC and LCMS showed that the r×n was complete. The reaction was quenched by water (29 μL). Anhydrous MgSO₄ (392 mg) was added. The mixture was filtered through celite, and the filtrate was concentrated. The resulting crude product was purified by normal phase column chromatography applying 0-15% ACN in EtOAc (each mobile phase contained 2.5% triethylamine) as the gradient to afford the title compound as a brownish off-white foam (1.26 g, 43.2% yield). ¹H NMR (600 MHz, Chloroform-d) δ 8.06 (s, 1H), 7.88 (d, J - 7.5 Hz, 2H), 7.55 (t, J - 7.3 Hz, 1H), 7.46 (t, J - 7.7 Hz, 2H), 7.41 (d, J=7.7 Hz, 2H), 7.33-7.27 (m, 4H), 7.19 (t, J=7.7 Hz, 2H), 7.14 (t, J=7.2 Hz, 1H), 6.79-6.71 (m, 4H), 6.29 (t, J=6.8 Hz, 1H), 5.59-5.48 (m, 1H), 5.11 (p, J=6.2, 5.3 Hz, 2H), 4.35 (dt, J=13.5, 6.7 Hz, 1H), 4.03 (q, J=5.2 Hz, 1H), 3.737 (s, 3H), 3.735 (s, 3H), 3.68 (dt, J=11.8, 5.7 Hz, 1H), 3.45 (dp, J=9.4, 4.8, 4.3 Hz, 2H), 3.37 (ddt, J=19.6, 9.3, 5.3 Hz, 3H), 3.27 (dd, J=10.0, 5.7 Hz, 1H), 3.00 (qd, J=9.9, 4.1 Hz, 1H), 2.24 (ddd, J=13.3, 7.3, 4.2 Hz, 1H), 1.87 (dtt, J=12.2, 8.1, 3.6 Hz, 1H), 1.76 (dddd, J=14.6, 11.7, 8.1, 4.8 Hz, 1H), 1.64 (h, J=7.6, 5.1 Hz, 1H), 1.29-1.25 (m, 6H), 1.12 (td, J=11.1, 10.5, 6.3 Hz, 1H); ³¹P NMR (243 MHz, Chloroform-d) δ 148.55; MS (ESI), 895.34 [M+H]⁺.

### Synthesis of (2R,3S,5R)-5-(6-benzamido-8-oxo-7,8-dihydro-9H-purin-9-yl)-2-((bis(4-methoxyphenyl)(phenyl)methoxy)methyl)tetrahydrofuran-3-yl (2-cyanoethyl) diisopropylphosphoramidite

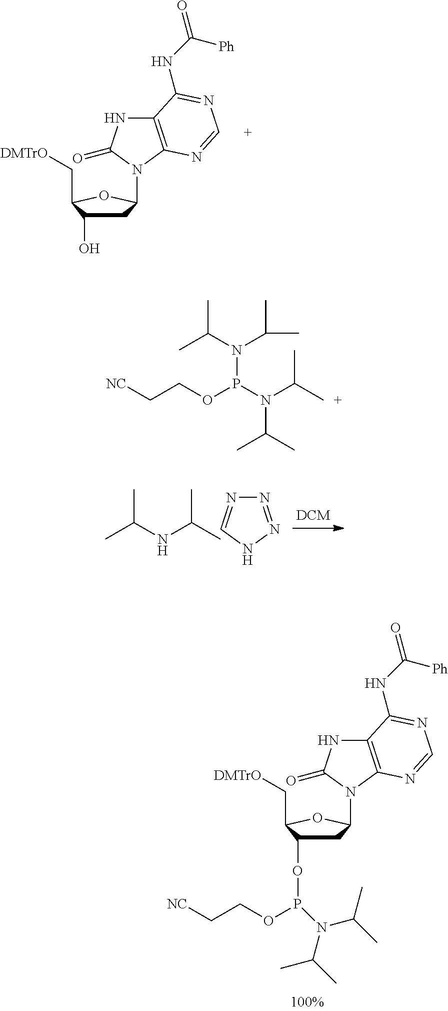

To a solution of dry N-[9-[(2R,4R,5R)-5-[[bis(4-methoxyphenyl)-phenyl-methoxy]methyl]-4-hydroxy-tetrahydrofuran-2-yl]-8-oxo-7H-purin-6-yl]benzamide (30.0 g, 44.5 mmol) in DCM (84 mL) at 0° C. was added 2-cyanoethyl N,N,N′,N′-tetraisopropylphosphorodiamidite (35.4 mL, 111 mmol) slowly. The solution was stirred for 5 min. Diisopropylammonium tetrazolide (2.29 g, 13.4 mmol) was added in one portion. Continued to stir at 0° C. for 5 min. The ice/water bath was removed. The resulting r×n solution was stirred at rt for 6 hr. TLC and LCMS showed that the r×n was complete. The mixture was filtered through celite, and the filtrate was concentrated. The resulting crude product was purified by normal phase column chromatography applying 30-70% EtOAc in hexanes (each mobile phase contained 1% triethylamine) as the gradient to afford the title compound as a white foam (39.3 g, 100% yield). ¹H NMR (600 MHz, Chloroform-d) 6 9.46 (s, 1H), 8.62 (d, J=3.8 Hz, 1H), 8.22-8.18 (m, 1H), 7.95-7.91 (m, 2H), 7.64 (t, J=7.4 Hz, 1H), 7.54 (t, J=7.6 Hz, 2H), 7.43 (dt, J=7.7, 3.4 Hz, 2H), 7.34-7.28 (m, 4H), 7.24-7.13 (m, 3H), 6.75 (q, J=9.1, 8.6 Hz, 4H), 6.42 (q, J=6.5 Hz, 1H), 4.94-4.80 (m, 1H), 4.25-4.18 (m, 1H), 3.89-3.77 (m, 1H), 3.77-3.74 (m, 6H), 3.71-3.64 (m, 1H), 3.64-3.54 (m, 2H), 3.45-3.37 (m, 1H), 3.36-3.28 (m, 2H), 2.61 (td, J - 6.5, 2.1 Hz, 1H), 2.46 (td, J=6.5, 4.5 Hz, 1H), 2.40-2.28 (m, 1H), 1.21-1.16 (m, 9H), 1.12 (d, J=6.8 Hz, 3H); “¹P NMR (243 MHz, Chloroform-d) δ 148.49, 148.30; MS (ESI), 874.20 [M+H]⁺.

### Synthesis of 3-((2R,3R,4R,5R)-5-((bis(4-methoxyphenyl)(phenyl)methoxy)methyl)-3-methoxy-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

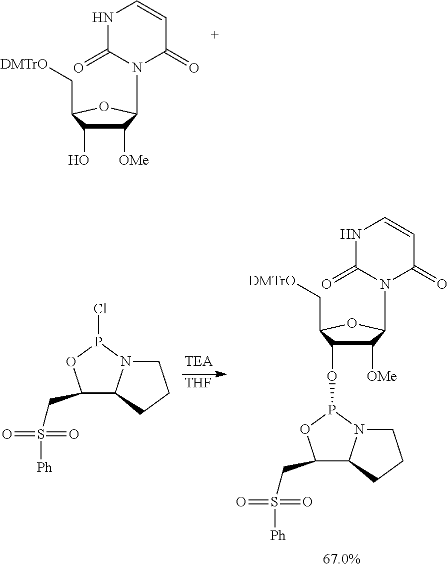

To a solution of dry 3-[(2R,3S,5R)-5-[[bis(4-methoxyphenyl)-phenyl-methoxy]methyl]-4-hydroxy-3-methoxy-tetrahydrofuran-2-yl]-1H-pyrimidine-2,4-dione (3.00 g, 5.35 mmol) in THF (18 mL) was added triethylamine (1.72 mL, 12.3 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.51 mL, 8.56 mmol) was added fast dropwise. The ice/water bath was removed. The resulting slurry was stirred at rt for 3 hr 15 min. TLC and LCMS showed that the r×n was complete. The reaction was quenched by water (48 μL). Anhydrous MgSO₄ (642 mg) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as a white foam (3.02 g, 67.0% yield). ¹H NMR (600 MHz, Chloroform-d) δ 8.99 (s, 1H), 7.88 (d, J=7.8 Hz, 2H), 7.59 (t, J=7.5 Hz, 1H), 7.51-7.44 (m, 4H), 7.34 (d, J=8.6 Hz, 4H), 7.23 (t, J=7.6 Hz, 2H), 7.16 (t, J=7.3 Hz, 1H), 6.82-6.74 (m, 5H), 6.35 (d, J=3.0 Hz, 1H), 5.57 (d, J=7.7 Hz, 1H), 5.08 (q, J=6.1 Hz, 1H), 4.87 (dt, J=9.5, 6.8 Hz, 1H), 4.35 (dd, J=6.2, 3.1 Hz, 1H), 4.09 (dq, J=6.8, 3.3 Hz, 1H), 3.75 (s, 6H), 3.66 (dq, J=11.6, 6.0 Hz, 1H), 3.43 (s, 3H), 3.41-3.31 (m, 4H), 3.19 (dd, J=10.4, 6.0 Hz, 1H), 2.88 (qd, J=9.7, 4.1 Hz, 1H), 1.80 (dtt, J=12.1, 8.0, 3.5 Hz, 1H), 1.75-1.66 (m, 1H), 1.61-1.54 (m, 1H), 1.10-1.03 (m, 1H); ³¹P NMR (243 MHz, Chloroform-d) δ 148.59; MS (ESI), 844.34 [M+H]⁺.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-5-ethylpyrimidine-2,4(1H,3H)-dione

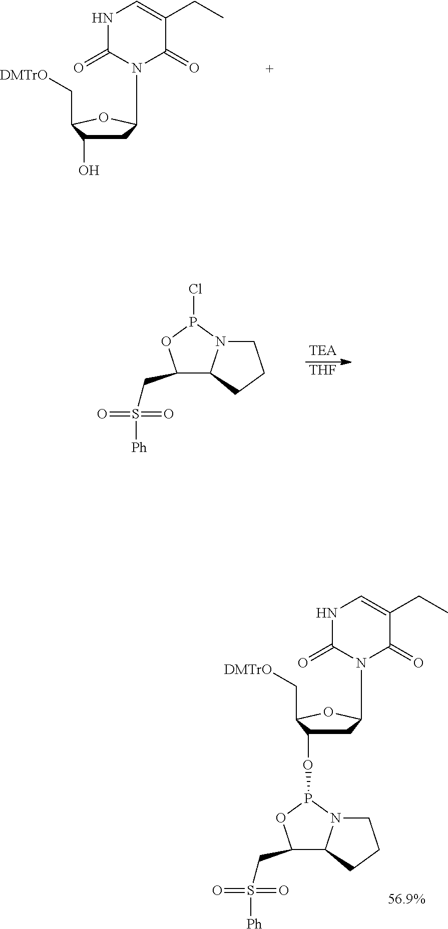

To a solution of dry 3-[(2R,4R,5R)-5-[[bis(4-methoxyphenyl)-phenyl-methoxy]methyl]-4-hydroxy-tetrahydrofuran-2-yl]-5-ethyl-1H-pyrimidine-2,4-dione (3.00 g, 5.37 mmol) in THF (18 mL) was added triethylamine (1.72 mL, 12.4 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.55 mL, 8.59 mmol) was added fast dropwise. The ice/water bath was removed. The resulting slurry was stirred at rt for 3 hr. TLC and LCMS showed that the r×n was complete. The reaction was quenched by water (48 μL). Anhydrous MgSO₄ (644 mg) was added. The mixture was filtered through celite, and the filtrate was concentrated. The resulting crude product was purified by normal phase column chromatography applying 30-90% EtOAc in hexanes (each mobile phase contained 2.5% triethylamine) as the gradient to afford the title compound as a white foam (2.57 g, 56.9% yield). ¹H NMR (600 MHz, Chloroform-d) δ 9.38 (s, 1H), 7.89-7.84 (m, 2H), 7.62-7.56 (m, 1H), 7.52-7.44 (m, 4H), 7.36 (dd, J=7.8, 1.4 Hz, 4H), 7.22 (dd, J=8.4, 7.0 Hz, 2H), 7.17-7.12 (m, 1H), 6.79-6.75 (m, 4H), 6.74 (dd, J=8.5, 5.1 Hz, 1H), 6.46 (s, 1H), 4.94-4.87 (m, 2H), 3.91 (td, J=6.0, 3.2 Hz, 1H), 3.745 (s, 3H), 3.740 (s, 3H), 3.60 (dq, J=11.5, 5.8 Hz, 1H), 3.43-3.25 (m, 5H), 2.99-2.93 (m, 1H), 2.91 (td, J=8.2, 4.1 Hz, 1H), 2.24 (ddd, J=13.6, 8.6, 5.4 Hz, 1H), 2.16 (qt, J=7.4, 1.4 Hz, 2H), 1.86-1.79 (m, 1H), 1.75-1.67 (m, 1H), 1.61 (dtd, J=12.2, 7.3, 6.7, 2.6 Hz, 1H), 1.10-1.03 (m, 1H), 0.97 (t, J=7.4 Hz, 3H); ³¹P NMR (243 MHz, Chloroform-d) δ 149.81; MS (ESI), 880.51 [M+K]⁺.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo [1,2-c][1,3,2]oxazaphosphol-1-yl)oxy)tetrahydrofuran-2-yl)-5-fluoropyrimidine-2,4(1H,3H)-dione

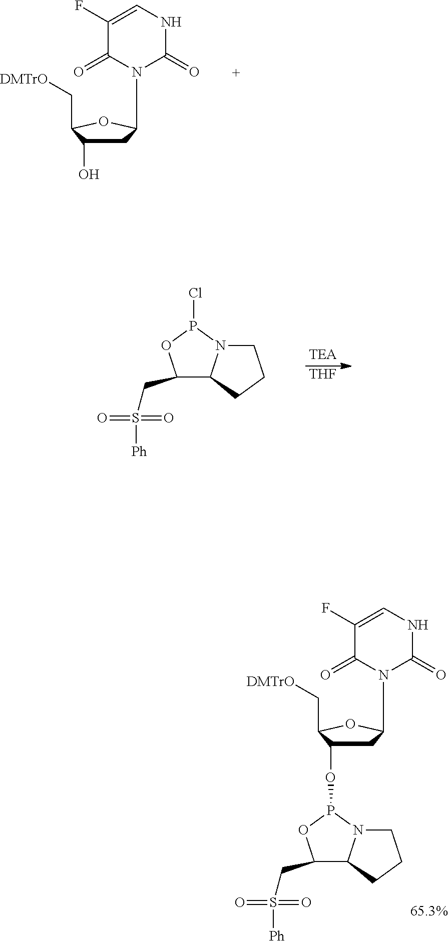

To a solution of dry 3-[(2R,4R,5R)-5-[[bis(4-methoxyphenyl)-phenyl-methoxy]methyl]-4-hydroxy-tetrahydrofuran-2-yl]-5-fluoro-1H-pyrimidine-2,4-dione (5.00 g, 9.11 mmol) in THF (30 mL) was added triethylamine (2.92 mL, 21.0 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 16.2 mL, 14.6 mmol) was added fast dropwise. The ice/water bath was removed. The resulting slurry was stirred at rt for 3.5 hr. TLC and LCMS showed that the r×n was complete. The reaction was quenched by water (82 μL). Anhydrous MgSO₄ (1.09 g) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as a white foam (4.95 g, 65.3% yield). ¹H NMR (600 MHz, Chloroform-d) δ 7.89-7.85 (m, 2H), 7.62-7.58 (m, 1H), 7.50 (t, J=7.8 Hz, 2H), 7.48-7.44 (m, 2H), 7.37-7.31 (m, 4H), 7.23 (dd, J=8.4, 7.0 Hz, 2H), 7.18-7.14 (m, 1H), 6.80-6.76 (m, 4H), 6.71 (d, J=4.2 Hz, 1H), 6.66 (dd, J=8.4, 5.0 Hz, 1H), 4.96 (dt, J=7.1, 5.5 Hz, 1H), 4.90-4.84 (m, 1H), 3.95 (td, J=6.0, 3.6 Hz, 1H), 3.75 (s, 3H), 3.74 (s, 3H), 3.61 (dq, J=9.9, 5.9 Hz, 1H), 3.43-3.26 (m, 5H), 2.96-2.90 (m, 1H), 2.88 (ddd, J=13.2, 8.0, 5.0 Hz, 1H), 2.62 (q, J=7.0 Hz, 1H), 2.26 (ddd, J=13.7, 8.5, 5.5 Hz, 1H), 1.86-1.79 (m, 1H), 1.76-1.67 (m, 1H), 1.63-1.57 (m, 1H); “¹P NMR (243 MHz, Chloroform-d) δ 149.29; ⁹F NMR (565 MHz, Chloroform-d) δ−164.78; MS (ESI), 870.18 [M+K]⁺.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-[1,2,4]triazolo[4,3-a]pyrazin-8(7H)-one

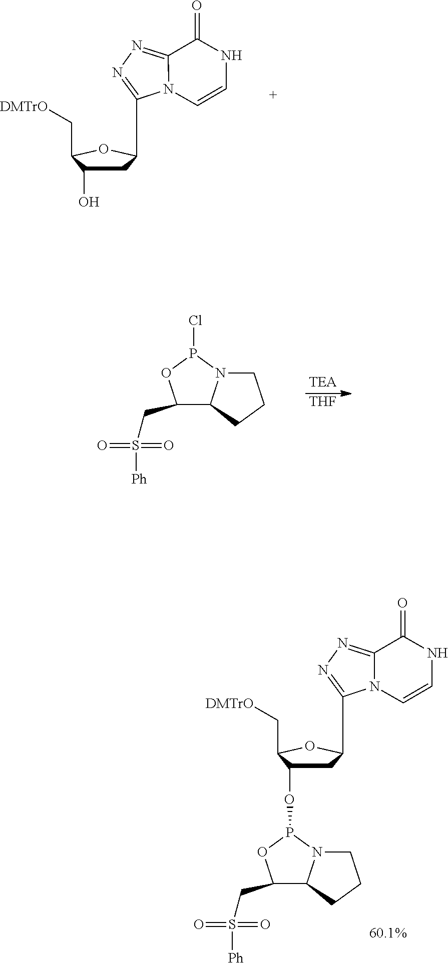

To a slurry of dry 3-[(2R,4R,5R)-5-[[bis(4-methoxyphenyl)-phenyl-methoxy]methyl]-4-hydroxy-tetrahydrofuran-2-yl]-7H-[1,2,4]triazolo[4,3-a]pyrazin-8-one (3.00 g, 5.41 mmol) in THF (30 mL) was added triethylamine (1.73 mL, 12.4 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90A4 in THF, 9.62 mL, 8.66 mmol) was added fast dropwise. The ice/water bath was removed. The resulting slurry was stirred at rt for 6 hr. TLC and LCMS showed that the r×n was complete. The reaction was quenched by water (49 μL). Anhydrous MgSO₄ (649 mg) was added. The mixture was filtered through celite, and the filtrate was concentrated. The resulting crude product was purified by normal phase column chromatography applying 0-100% ACN in EtOAc (each mobile phase contained 2.5% triethylamine) as the gradient to afford the title compound as a brownish off-white foam (2.72 g, 60.1% yield). ¹H NMR (600 MHz, Chloroform-d) δ 7.92-7.87 (m, 2H), 7.67 (d, J=5.7 Hz, 1H), 7.61-7.56 (m, 1H), 7.51-7.46 (m, 2H), 7.35-7.30 (m, 2H), 7.24 (dd, J=8.5, 6.8 Hz, 2H), 7.21 (dt, J=8.9, 2.2 Hz, 4H), 7.20-7.16 (m, 1H), 6.81-6.76 (m, 4H), 6.46 (d, J=5.7 Hz, 1H), 5.56 (dd, J=10.1, 5.8 Hz, 1H), 5.06 (dt, J=7.1, 5.5 Hz, 1H), 4.90 (tt, J=6.2, 2.0 Hz, 1H), 4.15 (q, J=3.4 Hz, 1H), 3.76 (s, 6H), 3.63 (ddd, J=12.1, 10.1, 5.9 Hz, 1H), 3.56-3.46 (m, 2H), 3.39 (dd, J=14.6, 5.2 Hz, 1H), 3.29-3.21 (m, 2H), 3.16 (tdd, J=10.3, 8.8, 4.0 Hz, 1H), 2.80 (ddd, J=13.3, 10.1, 6.0 Hz, 1H), 2.58 (ddd, J=13.4, 5.8, 1.7 Hz, 1H), 1.94-1.85 (m, 1H), 1.83-1.75 (m, 1H), 1.64 (tdd, J=7.7, 5.5, 2.5 Hz, 1H), 1.16-1.07 (m, 1H); ³¹P NMR (243 MHz, Chloroform-d) δ 153.51; MS (ESI), 836.43 [M−H]⁻.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-5-(prop-1-yn-1-yl)pyrimidine-2,4(1H,3H)-dione

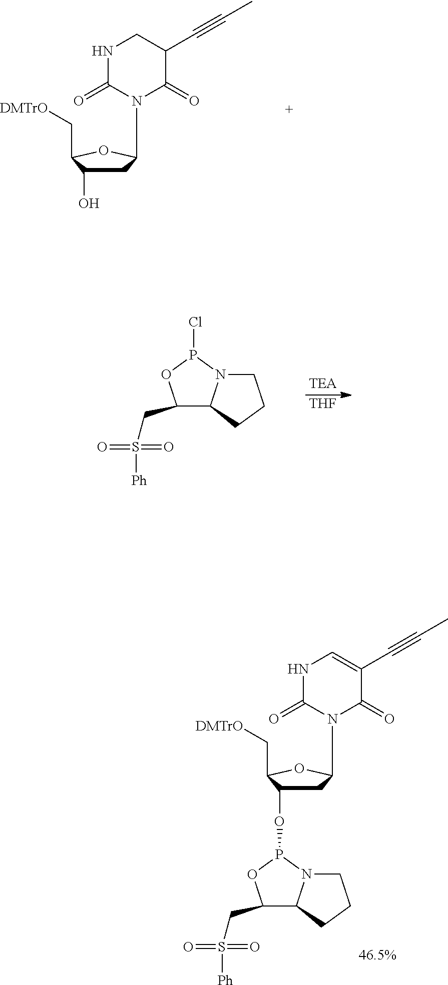

To a solution of dry 3-[(2R,4R,5R)-5-[[bis(4-methoxyphenyl)-phenyl-methoxy]methyl]-4-hydroxy-tetrahydrofuran-2-yl]-5-prop-1-ynyl-1H-pyrimidine-2,4-dione (3.00 g, 5.28 mmol) in THF (18 mL) was added triethylamine (1.69 mL, 12.1 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.38 mL, 8.44 mmol) was added fast dropwise. The ice/water bath was removed. The resulting slurry was stirred at rt for 2 hr 45 min. TLC and LCMS showed that the r×n was complete. The reaction was quenched by water (47 μL). Anhydrous MgSO₄ (633 mg) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a brownish off-white foam (2.09 g, 46.5% yield). ¹H NMR (600 MHz, Chloroform-d) δ 7.89-7.84 (m, 2H), 7.63-7.57 (m, 1H), 7.50 (t, J=7.8 Hz, 2H), 7.47-7.43 (m, 2H), 7.36-7.31 (m, 4H), 7.23 (t, J=7.7 Hz, 2H), 7.18-7.13 (m, 1H), 7.05 (s, 1H), 6.81-6.74 (m, 4H), 6.69 (dd, J=8.8, 4.5 Hz, 1H), 4.90-4.81 (m, 2H), 3.92 (td, J=6.6, 3.5 Hz, 1H), 3.755 (s, 3H), 3.750 (s, 3H), 3.61 (dq, J=9.9, 6.0 Hz, 1H), 3.43-3.33 (m, 3H), 3.33-3.25 (m, 2H), 2.95 (tdd, J=10.4, 8.8, 4.2 Hz, 1H), 2.79 (ddd, J=13.1, 8.3, 4.5 Hz, 1H), 2.25 (ddd, J=13.5, 8.9, 5.9 Hz, 1H), 2.02 (s, 3H), 1.82 (tp, J=10.9, 3.9, 3.3 Hz, 1H), 1.76-1.68 (m, 1H), 1.62 (dddd, J=9.4, 7.5, 5.7, 2.6 Hz, 1H), 1.10-1.01 (m, 1H); ³¹P NMR (243 MHz, Chloroform-d) δ 150.08; MS (ESI), 890.17 [M+K]⁺.

### Synthesis of 3-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-2,4-dioxo-1,2,3,4-tetrahydropyrimidine-5-carbonitrile

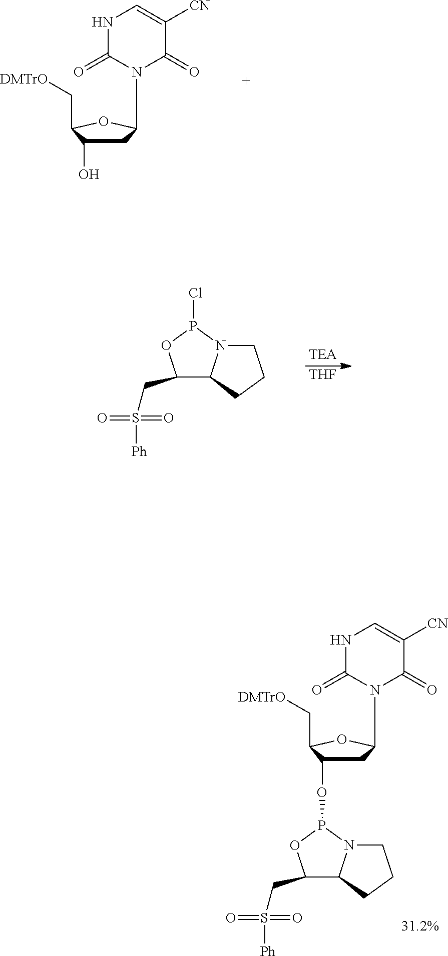

To a solution of dry 3-[(2R,4R,5R)-5-[[bis(4-methoxyphenyl)-phenyl-methoxy]methyl]-4-hydroxy-tetrahydrofuran-2-yl]-2,4-dioxo-1H-pyrimidine-5-carbonitrile (4.50 g, 8.10 mmol) in THF (27 mL) was added triethylamine (2.60 mL, 18.6 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 14.4 mL, 13.0 mmol) was added fast dropwise. The ice/water bath was removed. The resulting slurry was stirred at rt for 4.5 hr. TLC and LCMS showed that the r×n was complete. The reaction was quenched by water (73 L). Anhydrous MgSO₄ (972 mg) was added. The mixture was filtered through celite, and the filtrate was concentrated. The resulting crude product was purified by normal phase column chromatography applying 0-50% ACN in EtOAc (each mobile phase contained 2.5% triethylamine) as the gradient. The obtained product was not pure, so it was purified again by normal phase column chromatography applying 30-100% EtOAc in hexanes (each mobile phase contained 2.5% triethylamine) as the gradient to afford the title compound as a light-brown foam (2.12 g, 31.2% yield). ¹H NMR (600 MHz, Chloroform-d) δ 7.89-7.84 (m, 2H), 7.60 (t, J=7.4 Hz, 1H), 7.49 (t, J=7.7 Hz, 2H), 7.46 (d, J=7.6 Hz, 2H), 7.37-7.31 (m, 4H), 7.21 (t, J=7.6 Hz, 2H), 7.15 (t, J=7.2 Hz, 1H), 6.80-6.72 (m, 5H), 5.88 (s, 1H), 4.96-4.90 (m, 2H), 3.92 (td, J=6.6, 3.7 Hz, 1H), 3.76 (s, 6H), 3.61 (dq, J=11.6, 5.9 Hz, 1H), 3.40-3.31 (m, 3H), 3.31-3.24 (m, 2H), 2.89-2.82 (m, 1H), 2.79 (ddd, J=12.8, 8.4, 4.1 Hz, 1H), 2.21 (ddd, J=13.2. 9.2, 6.2 Hz, 1H), 1.81 (dtt, J=11.8, 7.9, 3.2 Hz, 1H), 1.75-1.65 (m, 1H), 1.62 (ddt, J=12.1, 9.6, 3.8 Hz, 1H), 1.10-1.01 (m, 1H); ³¹P NMR (243 MHz, Chloroform-d) δ 147.53; MS (ESI), 837.57 [M−H]⁻.

### Synthesis of 3-((S)-3-(bis(4-methoxyphenyl)(phenyl)methoxy)-2-(((1S,3S,3aS)-3-((phenylsulfonyl)methyl)tetrahydro-1H,3H-pyrrolo[1,2-c][1,3,2]oxazaphosphol-1-yl)oxy)propyl)pyrimidine-2,4(1H,3H)-dione

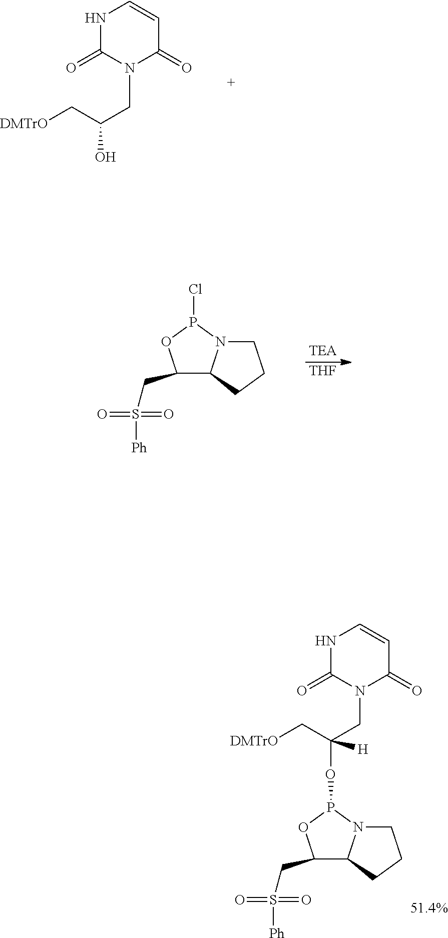

To a solution of dry 3-[(2S)-3-[bis(4-methoxyphenyl)-phenyl-methoxy]-2-hydroxy-propyl]-1H-pyrimidine-2,4-dione (2.50 g, 5.12 mmol) in THF (15 mL) was added triethylamine (1.64 mL, 11.8 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90Min THF, 9.10 mL, 8.19 mmol) was added fast dropwise. The ice/water bath was removed. The resulting slurry was stirred at rt for 2.5 hr. TLC and LCMS showed that the r×n was complete. The reaction was quenched by water (46 μL). Anhydrous MgSO₄ (614 mg) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as an off-white foam (2.03 g, 51.4% yield). ¹H NMR (600 MHz, Chloroform-d) δ 9.45 (s, 1H), 7.86-7.83 (m, 2H), 7.57 (t, J=7.5 Hz, 1H), 7.44 (t, J=8.1 Hz, 4H), 7.33 (dt, J=9.8, 2.6 Hz, 4H), 7.28-7.24 (m, 2H), 7.19 (t, J=7.4 Hz, 1H), 7.13 (d, J=7.7 Hz, 1H), 6.82 (dt, J=8.9, 2.9 Hz, 4H), 5.68 (d, J=7.7 Hz, 1H), 5.12 (q, J=6.2 Hz, 1H), 4.54 (tdd, J=11.2, 7.8, 4.7 Hz, 1H), 4.24 (dd, J=13.3, 9.1 Hz, 1H), 3.91 (dd, J=13.3, 4.0 Hz, 1H), 3.80-3.76 (m, 6H), 3.64 (dq, J =9.4, 5.9 Hz, 1H), 3.43 (dd, J=14.4, 6.7 Hz, 1H), 3.41-3.35 (m, 1H), 3.31 (dd, J=14.4, 6.1 Hz, 1H), 3.22 (dd, J=9.6, 5.7 Hz, 1H), 3.08 (dd, J=9.6, 5.4 Hz, 1H), 2.97 (dtd, J=14.6, 10.2, 9.4, 4.5 Hz, 1H), 1.77 (dp, J =12.2, 4.1 Hz, 1H), 1.73-1.67 (m, 1H), 1.63 (dtd, J=12.1, 6.1, 3.2 Hz, 1H), 1.12-1.05 (m, 1H); ³¹P NMR (243 MHz, Chloroform-d) δ 151.28; MS (ESI), 810.28 [M+K]⁺.

### Synthesis of 3-((R)-3-(bis(4-methoxyphenyl)(phenyl)methoxy)-2-(((1S,3S,3aS)-3-((phenylsulfonyl)methyl)tetrahydro-1H,3H-pyrrolo[1,2-c][1,3,2]oxazaphosphol-1-yl)oxy)propyl)pyrimidine-2,4(1H,3H)-dione

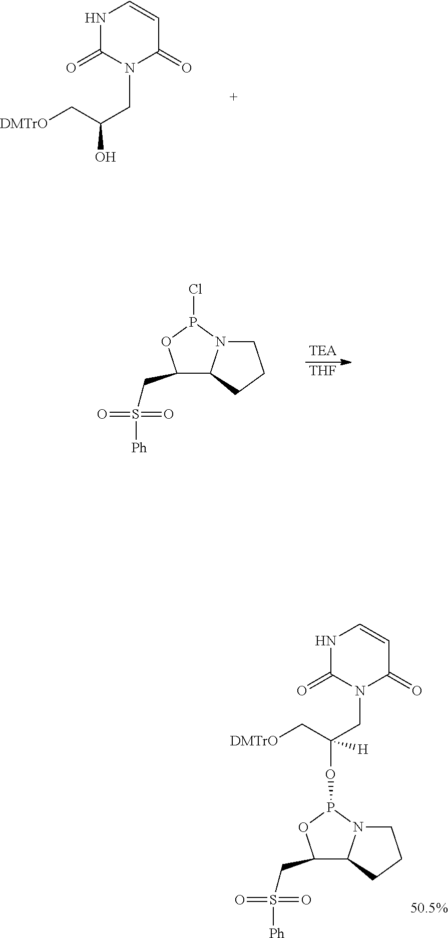

To a solution of dry 3-[(2R)-3-[bis(4-methoxyphenyl)-phenyl-methoxy]-2-hydroxy-propyl]-1H-pyrimidine-2,4-dione (2.00 g, 4.09 mmol) in THF (12 mL) was added triethylamine (1.31 mL, 9.42 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90Min THF, 7.28 mL, 6.55 mmol) was added fast dropwise. The ice/water bath was removed. The resulting slurry was stirred at rt for 4 hr. TLC and LCMS showed that the r×n was complete. The reaction was quenched by water (37 μL). Anhydrous MgSO₄ (491 mg) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as a white foam (1.60 g, 50.5% yield). ¹H NMR (600 MHz, Chloroform-d) δ 8.98 (s, 1H), 7.97 (dd, J=8.1, 1.4 Hz, 2H), 7.69-7.65 (m, 1H), 7.58 (t, J=7.7 Hz, 2H), 7.49-7.44 (m, 2H), 7.34 (ddd, J=9.6, 6.0, 2.8 Hz, 4H), 7.25 (t, J=7.6 Hz, 2H), 7.21-7.16 (m, 2H), 6.85-6.77 (m, 4H), 5.59 (d, J=7.7 Hz, 1H), 5.06 (q, J=6.1 Hz, 1H), 4.61 (ddq, J=14.1, 7.3, 3.6 Hz, 1H), 4.17 (dd, J=13.3, 9.6 Hz, 1H), 3.83-3.79 (m, 1H), 3.78 (s, 6H), 3.57 (dq, J=9.9, 6.0 Hz, 1H), 3.50-3.41 (m, 2H), 3.38 (dd, J=14.5, 5.8 Hz, 1H), 3.24 (dd, J=9.7, 7.1 Hz, 1H), 3.11-3.04 (m, 2H), 1.80 (ddd, J - 11.7, 7.8, 3.4 Hz, 1H), 1.73-1.65 (m, 1H), 1.50 (qd, J - 7.3, 2.5 Hz, 1H), 1.10-1.02 (m, 1H); ³¹P NMR (243 MHz, Chloroform-d) δ 157.59; MS (ESI), 770.18 [M−H]⁻.

### Synthesis of 3-((2R,3S,4R,5R)-5-((bis(4-methoxyphenyl)(phenyl)methoxy)methyl)-3-fluoro-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

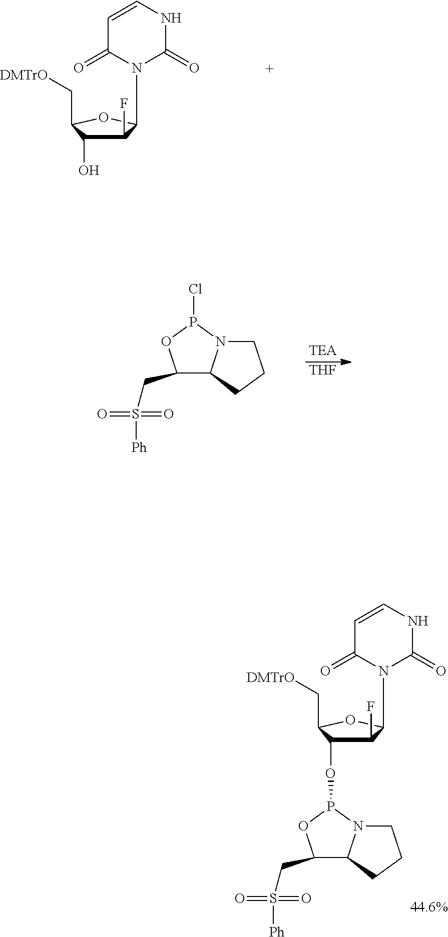

To a solution of dry 3-[(2R,4S,5R)-5-[[bis(4-methoxyphenyl)-phenyl-methoxy]methyl]-3-fluoro-4-hydroxy-tetrahydrofuran-2-yl]-1H-pyrimidine-2,4-dione (5.00 g, 9.11 mmol) in THF (30 mL) was added triethylamine (2.79 mL, 20.1 mmol). The r×n solution was cooled to −20° C. (3S,3aS)-3-(benzenesulfonylmethyl)-1-chloro-3a,4,5,6-tetrahydro-3H-pyrrolo[1,2-c][1,3,2]oxazaphosphole (0.90M in THF, 16.2 mL, 14.6 mmol) was added fast dropwise. The resulting slurry was allowed to slowly warm to rt and continued to stir at rt for 3 hr. LCMS showed minor nucleoside remained. Stirred for another 4 hr. The reaction was quenched by water (82 μL). Anhydrous MgSO₄ (1.09 g) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient to afford the title compound as a white foam (3.38 g, 44.6% yield). ¹H NMR (600 MHz, Chloroform-d) δ 9.16 (s, 1H), 7.88-7.84 (m, 2H), 7.62-7.57 (m, 1H), 7.50-7.43 (m, 4H), 7.37-7.31 (m, 4H), 7.23 (t, J=7.7 Hz, 2H), 7.18-7.13 (m, 1H), 6.83-6.72 (m, 6H), 5.57 (d, J=7.7 Hz, 1H), 5.24 (ddd, J=54.1, 7.9, 5.6 Hz, 1H), 5.10-5.00 (m, 1H), 4.97 (q, J=6.2 Hz, 1H), 3.88 (ddd, J=9.3, 7.6, 2.2 Hz, 1H), 3.75 (s, 3H), 3.74 (s, 3H), 3.62 (dq, J=9.6, 5.9 Hz, 1H), 3.54 (dd, J=10.2, 7.8 Hz, 1H), 3.36-3.26 (m, 4H), 2.79 (qd, J=9.4, 4.0 Hz, 1H), 1.80 (pt, J=8.8, 4.3 Hz, 1H), 1.74-1.66 (m, 1H), 1.66-1.60 (m, 1H), 1.10-1.01 (m, 1H); ³¹P NMR (243 MHz, Chloroform-d) δ 148.18 (d, J=2.2 Hz); ⁹F NMR (565 MHz, Chloroform-d) δ −199.71 (d, J=2.7 Hz); MS (ESI), 830.56 [M−H]⁻.

### Synthesis of 3-((2S,3S,4R,5R)-5-((bis(4-methoxyphenyl)(phenyl)methoxy)methyl)-3-fluoro-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

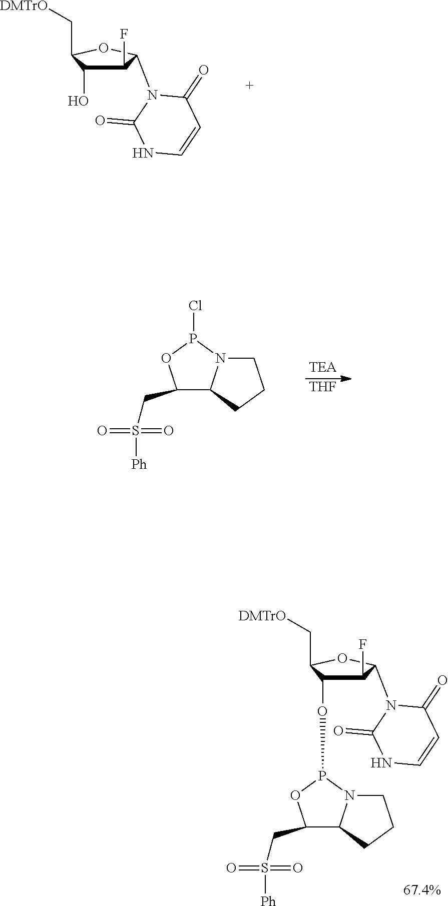

To a solution of dry 3-[(2S,4S,5R)-5-[[bis(4-methoxyphenyl)-phenyl-methoxy]methyl]-3-fluoro-4-hydroxy-tetrahydrofuran-2-yl]-1H-pyrimidine-2,4-dione (10.0 g, 18.2 mmol) in THF (60 mL) was added triethylamine (5.59 mL, 40.1 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 34.4 mL, 31.0 mmol) was added fast dropwise. The ice/water bath was removed. The resulting slurry was stirred at rt for 2.5 hr. TLC and LCMS showed that the r×n was complete. The reaction was quenched by water (164 μL). Anhydrous MgSO₄ (2.18 g) was added. The mixture was filtered through celite, and the filtrate was concentrated. The resulting crude product was purified by normal phase column chromatography applying 40-100% EtOAc in hexanes (each mobile phase contained 2.5% triethylamine) as the gradient. The less pure product fractions were combined, concentrated, and purified one more time applying the same gradient. The combined pure product fractions from the two column purifications were combined, concentrated, and further dried on high vacuum to afford the title compound as a white foam (10.2 g, 67.4% yield). ¹H NMR (600 MHz, Chloroform-d) δ 7.91-7.86 (m, 2H), 7.62-7.56 (m, 1H), 7.50 (t, J=7.8 Hz, 2H), 7.48-7.44 (m, 2H), 7.37-7.31 (m, 4H), 7.28 (t, J=7.7 Hz, 2H), 7.21-7.18 (m, 1H), 7.17 (d, J=7.9 Hz, 1H), 6.84-6.79 (m, 4H), 6.71 (dd, J=21.1, 4.2 Hz, 1H), 5.82 (dt, J=56.9, 4.5 Hz, 1H), 5.73 (d, J=7.7 Hz, 1H), 5.08-4.95 (m, 2H), 4.50 (dt, J=8.2, 3.0 Hz, 1H), 3.77 (s, 6H), 3.62 (dq, J=9.7, 6.0 Hz, 1H), 3.42-3.33 (m, 3H), 3.30 (dd, J=14.6, 6.0 Hz, 1H), 3.05 (dd, J=10.8, 3.9 Hz, 1H), 2.93-2.84 (m, 1H), 1.83 (dtd, J=12.2, 8.5, 8.0, 3.9 Hz, 1H), 1.76-1.67 (m, 1H), 1.66-1.59 (m, 1H), 1.11-1.03 (m, 1H); ³¹P NMR (243 MHz, Chloroform-d) δ 149.78; ¹⁹F NMR (565 MHz, Chloroform-d) δ −186.62; MS (ESI), 830.46 [M−H]⁻.

### Synthesis of 9-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-6-(dimethylamino)-7,9-dihydro-8H-purin-8-one

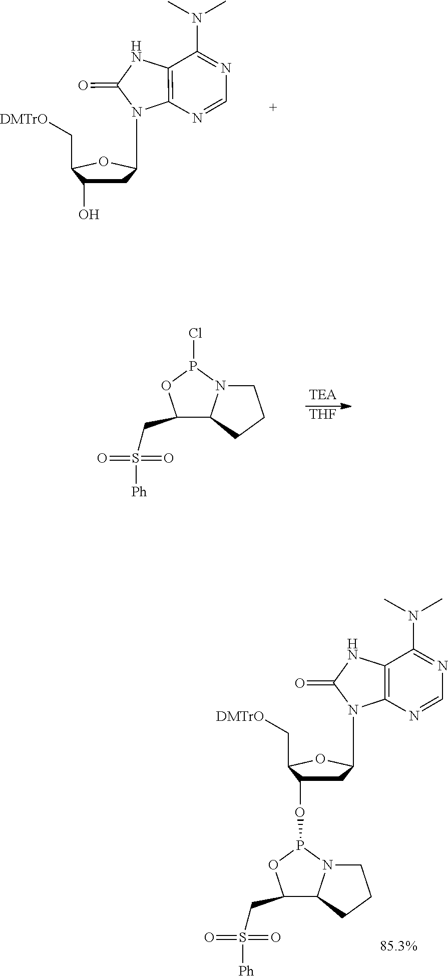

To a solution of dry 9-[(2R,4R,5R)-5-[[bis(4-methoxyphenyl)-phenyl-methoxy]methyl]-4-hydroxy-tetrahydrofuran-2-yl]-6-(dimethylamino)-7H-purin-8-one (3.00 g, 5.02 mmol) in THF (18 mL) was added triethylamine (1.54 mL, 11.0 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.48 mL, 8.53 mmol) was added fast dropwise. The ice/water bath was removed. The resulting slurry was stirred at rt for 3 hr. TLC and LCMS showed that the r×n was complete. The reaction was quenched by water (45 μL). Anhydrous MgSO₄ (602 mg) was added. The mixture was filtered through celite, and the filtrate was concentrated. The resulting crude product was purified by normal phase column chromatography applying 40-100% EtOAc in hexanes (mobile phase A contained 1% triethylamine, mobile phase B contained 2.5% triethylamine) as the gradient to afford the title compound as a white foam (3.77 g, 85.3% yield). ¹H NMR (600 MHz, Chloroform-d) δ 10.85 (s, 1H), 8.05 (s, 1H), 7.88 (d, J=7.6 Hz, 2H), 7.58-7.54 (m, 1H), 7.48 (t, J=7.6 Hz, 2H), 7.41 (d, J=7.8 Hz, 2H), 7.29 (dd, J=8.8, 2.1 Hz, 4H), 7.19 (t, J=7.6 Hz, 2H), 7.15-7.11 (m, 1H), 6.73 (t, J=8.5 Hz, 4H), 6.35 (t, J=6.9 Hz, 1H), 5.06 (p, J=5.9 Hz, 2H), 4.04 (q, J=5.1 Hz, 1H), 3.72 (s, 3H), 3.71 (s, 3H), 3.72-3.67 (m, 1H), 3.54-3.42 (m, 2H), 3.41-3.33 (m, 3H), 3.27 (dd, J=10.0, 5.7 Hz, 1H), 3.22 (s, 6H), 3.10 (qd, J=9.8, 3.9 Hz, 1H), 2.30-2.23 (m, 1H), 1.89 (ddq, J=12.4, 8.7, 4.7, 3.8 Hz, 1H), 1.82-1.74 (m, 1H), 1.67 (qd, J=7.5, 2.5 Hz, 1H), 1.14 (tq, J=19.2, 10.2, 8.7 Hz, 1H); ³¹P NMR (243 MHz, Chloroform-d) δ 151.22; MS (ESI), 881.26 [M+H]⁺.

### Synthesis of 1-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo [1,2-c][1,3,2]oxazaphosphol-1-yl)oxy)tetrahydrofuran-2-yl)-5-(prop-i-yn-1-yl)pyrimidine-2,4(1H,3H)-dione

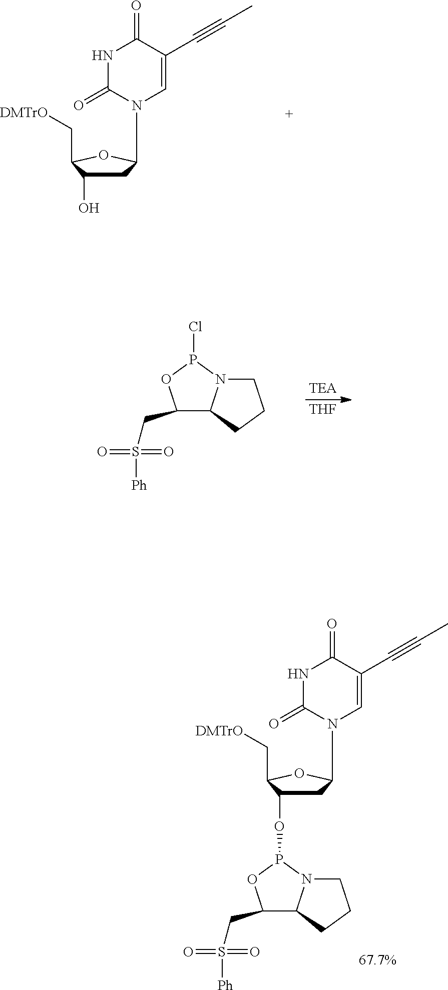

To a solution of dry 1-[(2R,4R)-5-[[bis(4-methoxyphenyl)-phenyl-methoxy]methyl]-4-hydroxy-tetrahydrofuran-2-yl]-5-prop-1-ynyl-pyrimidine-2,4-dione (3.00 g, 5.28 mmol) in THF (18 mL) was added triethylamine (1.62 mL, 11.6 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 9.97 mL, 8.97 mmol) was added fast dropwise. The ice/water bath was removed. The resulting slurry was stirred at rt for 3 hr. TLC and LCMS showed that the r×n was complete. The reaction was quenched by water (47 μL). Anhydrous MgSO₄ (633 mg) was added. The mixture was filtered through celite, and the filtrate was concentrated. The resulting crude product was purified by normal phase column chromatography applying 40-100% EtOAc in hexanes (mobile phase A contained 1% triethylamine, mobile phase B contained 2.5% triethylamine) as the gradient to afford the title compound as a white foam (3.04 g, 67.7% yield). ¹H NMR (600 MHz, Chloroform-d) δ 8.24 (s, 1H), 8.00 (s, 1H), 7.90-7.85 (m, 2H), 7.63-7.58 (m, 1H), 7.49 (t, J=7.7 Hz, 2H), 7.46-7.42 (m, 2H), 7.37-7.33 (m, 4H), 7.31 (t, J=7.6 Hz, 2H), 7.24-7.20 (m, 1H), 6.88-6.83 (m, 4H), 6.29 (dd, J=8.2, 5.6 Hz, 1H), 4.96 (dt, J=7.1, 5.4 Hz, 1H), 4.77 (ddd, J=11.1, 5.0, 2.3 Hz, 1H), 4.06 (q, J=2.7 Hz, 1H), 3.79 (s, 6H), 3.60 (dq, J=9.8, 5.9 Hz, 1H), 3.54-3.47 (m, 1H), 3.46 (dd, J=14.4, 7.2 Hz, 1H), 3.39-3.29 (m, 3H), 3.19-3.12 (m, 1H), 2.54 (ddd, J=13.8, 5.7, 2.0 Hz, 1H), 2.26 (ddd, J=13.9, 8.2, 6.0 Hz, 1H), 1.89 (tt, J=8.3, 4.0 Hz, 1H), 1.83-1.75 (m, 1H), 1.69 (s, 3H), 1.64 (dt, J=12.1, 7.4 Hz, 1H), 1.15-1.06 (m, 1H); ³¹P NMR (243 MHz, Chloroform-d) δ 153.88; MS (ESI), 850.46[M−H]⁻).

### Synthesis of 1-((2R,3R,4R,5R)-5-((bis(4-methoxyphenyl)(phenyl)methoxy)methyl)-3-fluoro-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-5-(prop-i-yn-1-yl)pyrimidine-2,4(1H,3H)-dione

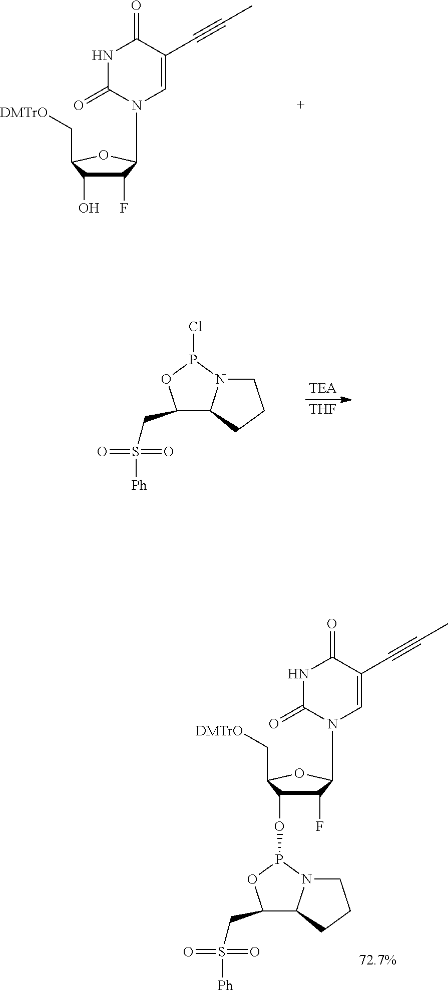

To a solution of dry 1-[(3S,4S)-5-[[bis(4-methoxyphenyl)-phenyl-methoxy]methyl]-3-fluoro-4-hydroxy-tetrahydrofuran-2-yl]-5-prop-1-ynyl-pyrimidine-2,4-dione (2.00 g, 3.41 mmol) in THF (12 mL) was added triethylamine (1.09 mL, 7.84 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 6.44 mL, 5.80 mmol) was added fast dropwise. The ice/water bath was removed. The resulting slurry was stirred at rt for 3 hr. TLC and LCMS showed that the r×n was complete. The reaction was quenched by water (31 L). Anhydrous MgSO₄ (409 mg) was added. The mixture was filtered through celite, and the filtrate was concentrated. The resulting crude product was purified by normal phase column chromatography applying 40-100% EtOAc in hexanes (mobile phase A contained 1% triethylamine, mobile phase B contained 2.5% triethylamine) as the gradient to afford the title compound as a brownish off-white foam (2.16 g, 72.7% yield).

¹H NMR (600 MHz, Chloroform-d) δ 7.92 (s, 1H), 7.90-7.86 (m, 2H), 7.62 (t, J=7.5 Hz, 1H), 7.50 (t, J=7.7 Hz, 2H), 7.45-7.41 (m, 2H), 7.34 (dd, J=8.9, 3.0 Hz, 4H), 7.29 (t, J=7.7 Hz, 2H), 7.22 (t, J=7.3 Hz, 1H), 6.84 (d, J=8.8 Hz, 4H), 6.00 (dd, J=15.5, 2.9 Hz, 1H), 5.15 (ddd, J=52.0, 4.6, 2.8 Hz, 1H), 5.06-5.01 (m, 1H), 4.74 (dddd, J=15.1, 8.5, 6.4, 4.5 Hz, 1H), 4.18-4.14 (m, 1H), 3.783 (s, 3H), 3.781 (s, 3H), 3.71 3.65 (m, 1H), 3.53-3.43 (m, 3H), 3.41-3.34 (m, 2H), 3.11 (qd, J=10.2, 4.1 Hz, 1H), 1.86 (dqt, J=11.0, 7.6, 3.4 Hz, 1H), 1.82-1.73 (m, 1H), 1.68-1.58 (m, 1H), 1.64 (s, 3H), 1.15-1.07 (m, 1H); “P NMR (243 MHz, Chloroform-d) δ 154.11 (d, J=12.8 Hz); ¹⁹F NMR (565 MHz, Chloroform-d) δ −201.92 (d, J=12.6 Hz); MS (EST), 868.37 [M−H]⁻).

### Synthesis of N-(1-((2R,3S,4R,5R)-5-((bis(4-methoxyphenyl)(phenyl)methoxy)methyl)-3-fluoro-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-2-oxo-1,2-dihydropyrimidin-4-yl)acetamide

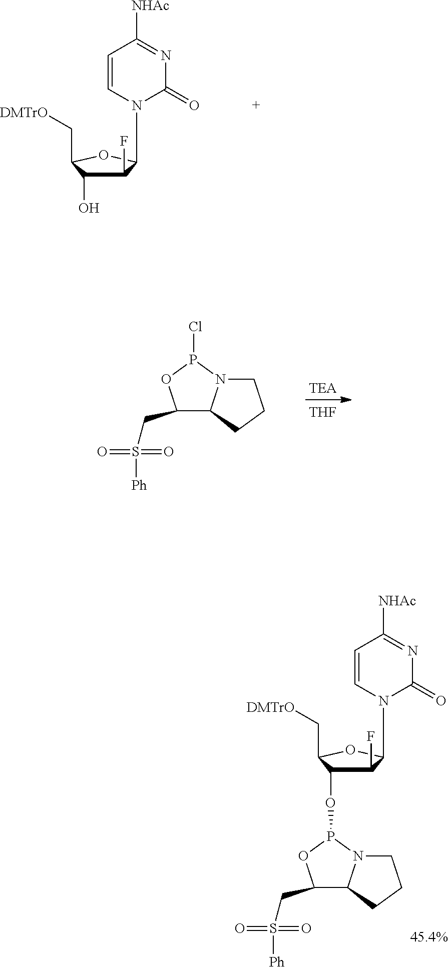

To a solution of dry N-[1-[(2R,3R,5R)-5-[[bis(4-methoxyphenyl)-phenyl-methoxy]methyl]-3-fluoro-4-hydroxy-tetrahydrofuran-2-yl]-2-oxo-pyrimidin-4-yl]acetamide (2.00 g, 3.39 mmol) in THF (12 mL) was added triethylamine (1.09 mL, 7.80 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 6.41 mL, 5.77 mmol) was added fast dropwise. The ice/water bath was removed. The resulting slurry was stirred at rt for 4 hr. TLC and LCMS showed that the r×n was not complete. Additional triethylamine (0.31 mL, 2.2 mmol) was added. Additional (3S,3aS)-3-(benzenesulfonylmethyl)-1-chloro-3a,4,5,6-tetrahydro-3H-pyrrolo[1,2-c][1,3,2]oxazaphosphole (0.90M in THF, 1.88 mL, 1.70 mmol) was added fast dropwise. The slurry was stirred at rt for another 2 hr. The mixture was filtered through celite, and the filtrate was concentrated. The resulting crude product was purified by normal phase column chromatography applying 40-100% EtOAc in hexanes (mobile phase A contained 1% triethylamine, mobile phase B contained 2.5% triethylamine) as the gradient to afford the title compound as a white foam (1.35 g, 45.4% yield). ¹H NMR (600 MHz, Chloroform-d) δ 9.20 (s, 1H), 7.93-7.88 (m, 2H), 7.87 (d, J - 7.6 Hz, 1H), 7.61 (t, J - 7.4 Hz, 1H), 7.52 (t, J=7.7 Hz, 2H), 7.47-7.41 (m, 2H), 7.36-7.28 (m, 7H), 7.24 (t, J=7.3 Hz, 1H), 6.85 (d, J=8.6 Hz, 4H), 6.21 (dd, J=19.2, 3.2 Hz, 1H), 5.14 (dd, J=51.0, 3.3 Hz, 1H), 5.00 (q, J=6.0 Hz, 1H), 4.69 (ddd, J=17.9. 9.3, 3.5 Hz, 1H), 4.07 (q, J=4.4 Hz, 1H), 3.80 (s, 6H), 3.63 (dq, J=11.7, 6.0 Hz, 1H), 3.52-3.43 (m, 2H), 3.41 (dd, J=10.4, 4.3 Hz, 1H), 3.39-3.32 (m, 2H), 3.04 (qd, J=9.9, 4.0 Hz, 1H), 2.26 (s, 3H), 1.89 (pd, J=7.5, 3.1 Hz, 1H), 1.78 (h, J=7.9 Hz, 1H), 1.66 (ddt, J=12.7, 9.9, 3.9 Hz, 1H), 1.16-1.06 (m, 1H); ³¹P NMR (243 MHz, Chloroform-d) δ 151.23; ¹⁹F NMR (565 MHz, Chloroform-d) δ −197.00; MS (ESI), 873.21 [M+H]⁺.

### Synthesis of 9-((2R,3R,4R,5R)-5-((bis(4-methoxyphenyl)(phenyl)methoxy)methyl)-3-methoxy-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-1,9-dihydro-6H-purin-6-one

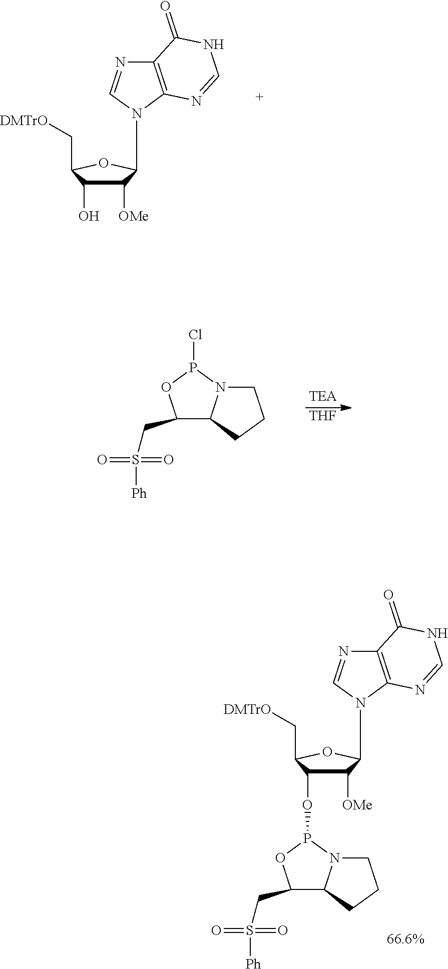

To a cloudy solution of dry 5′-DMT-2′-O-Methyl Inosine (5.00 g, 8.55 mmol) in THF (37.5 mL) was added triethylamine (2.86 mL, 20.5 mmol). (3S,3aS)-3-(benzenesulfonylmethyl)-1-chloro-3a,4,5,6-tetrahydro-3H-pyrrolo[1,2-c][1,3,2]oxazaphosphole (0.90M in THF, 17.1 mL, 15.4 mmol) was added fast dropwise. The resulting slurry was stirred at rt for 3.5 hr. TLC and LCMS showed that minor nucleoside starting material remained. The r×n mixture was stirred for another 2.5 hr. Since the r×n mixture became much thicker, additional THF (75 mL) was added. The reaction was quenched by water (77 μL). Anhydrous MgSO₄ (1.03 g) was added. The mixture was filtered through celite, and the filtrate was concentrated. The resulting crude product was purified by normal phase column chromatography applying 0-100% ACN in DCM (each mobile phase contained 1% triethylamine) as the gradient to afford the title compound as a white foam (4.94 g, 66.6% yield). ¹H NMR (600 MHz, Chloroform-d) δ 12.62 (s, 1H), 8.04 (s, 1H), 7.96 (s, 1H), 7.87 (dd, J=8.1, 1.4 Hz, 2H), 7.59-7.55 (m, 1H), 7.46 (t, J=7.8 Hz, 2H), 7.45-7.41 (m, 2H), 7.32 (dd, J=8.9, 2.3 Hz, 4H), 7.27 (t, J=7.8 Hz, 2H), 7.23-7.19 (m. 1H), 6.81 (dd, J=8.9, 3.1 Hz, 4H), 6.05 (d. J=6.0 Hz, 1H), 5.13-5.07 (m, 1H), 4.75 (dt, J=9.2, 4.0 Hz, 1H), 4.57 (t, J=5.5 Hz, 1H), 4.26 (q, J=3.7 Hz, 1H), 3.77 (s, 3H), 3.76 (s, 3H), 3.65 (dq, J=11.6, 6.0 Hz, 1H), 3.53-3.45 (m, 3H), 3.43 (s, 3H), 3.42-3.39 (m, 1H), 3.36 (dd, J=10.7, 4.1 Hz, 1H), 3.15-3.08 (m, 1H), 1.88 (ddq, J=11.3, 7.5, 4.1, 3.1 Hz, 1H), 1.81-1.73 (m, 1H), 1.66 (ddt, J=12.6, 9.7, 3.9 Hz, 1H), 1.17-1.07 (m, 1H).; “P NMR (243 MHz, Chloroform-d) δ 154.61; MS (ESI), 868.28 [M+H]⁺.

### Synthesis of 1-(9-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)-3-methylurea

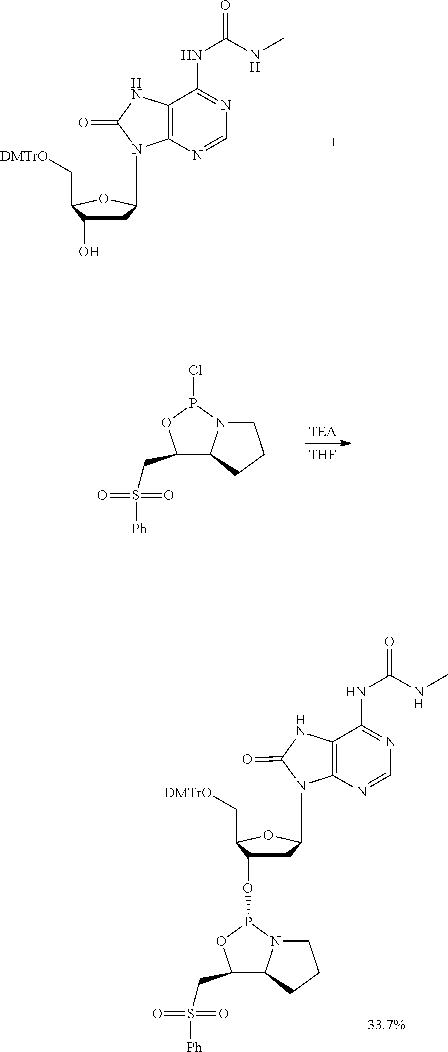

To a solution of dry 1-[9-[(2R,4R,5R)-5-[[bis(4-methoxyphenyl)-phenyl-methoxy]methyl]-4-hydroxy-tetrahydrofuran-2-yl]-8-oxo-7H-purin-6-yl]-3-methyl-urea (1.00 g, 1.60 mmol) in THF (6 mL) was added triethylamine (0.49 mL, 3.51 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.90M in THF, 2.84 mL, 2.55 mmol) was added dropwise. The ice/water bath was removed. The resulting slurry was stirred at rt for 3 hr. TLC showed that minor nucleoside starting material remained. The r×n mixture was stirred for another 3 hr. The reaction was quenched by water (14 L). Anhydrous MgSO₄ (191 mg) was added. The mixture was filtered through celite, and the filtrate was concentrated. The resulting crude product was purified by normal phase column chromatography applying 0-100% ACN in EtOAc (each mobile phase contained 1% triethylamine) as the gradient to afford the title compound as a brownish off-white foam (0.489 g, 33.7% yield). ¹H NMR (600 MHz, Chloroform-d) δ 10.55 (s, 1H), 9.37 (s, 1H), 8.17 (s, 1H), 7.91-7.87 (m, 2H), 7.61-7.57 (m, 1H), 7.50 (t, J=7.8 Hz, 2H), 7.45-7.41 (m, 2H), 7.33-7.28 (m, 4H), 7.21 (dd, J=8.5, 6.7 Hz, 2H), 7.15 (dd, J=8.4, 5.9 Hz, 1H), 6.77-6.72 (m, 4H), 6.34 (t, J=6.8 Hz, 1H), 5.12-5.03 (m, 2H), 4.03 (q, J=4.9 Hz, 1H), 3.75 (s, 3H), 3.74 (s, 3H), 3.68 (dq, J=10.0, 6.0 Hz, 1H), 3.50-3.43 (m, 2H), 3.39-3.34 (m, 2H), 3.32-3.23 (m, 2H), 3.06 (d, J=4.6 Hz, 3H), 3.02 (td, J=10.1, 9.5, 4.0 Hz, 1H), 2.29 (ddd, J=13.4, 7.3, 4.3 Hz, 1H), 1.87 (dtt, J=11.8, 7.9, 3.2 Hz, 1H), 1.80-1.72 (m, 1H), 1.66 (ddt, J=12.0, 7.5, 4.2 Hz, 1H), 1.15-1.07 (m, 1H); ³¹P NMR (243 MHz, Chloroform-d) δ 149.97; MS (ESI), 908.47 [M−H]⁻).

### Synthesis of 3-((2R,5S,6R)-6-((bis(4-methoxyphenyl)(phenyl)methoxy)methyl)-5-hydroxytetrahydro-2H-pyran-2-yl)pyrimidine-2,4(1H,3H)-dione (WV-NU-279)

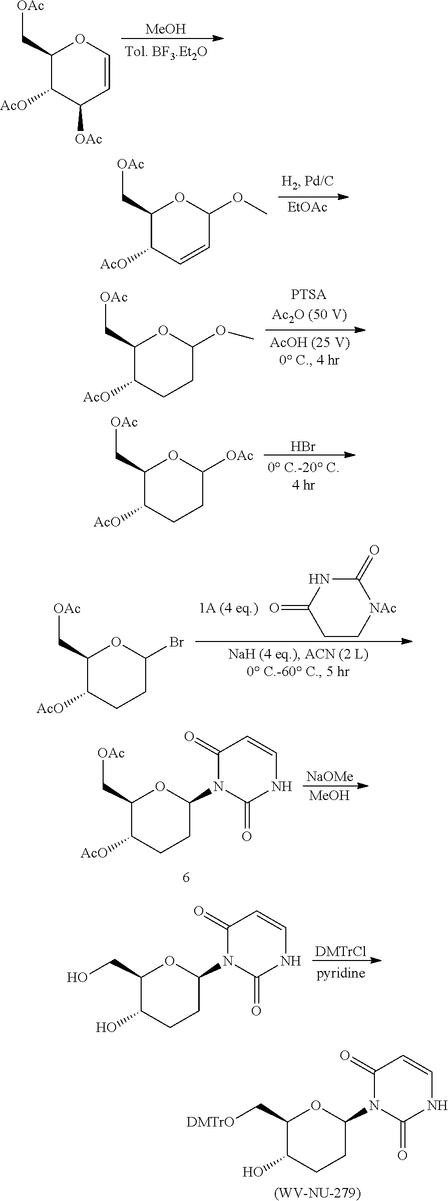

Step 1. BORON TRIFLUORIDE DIETHYL ETHERATE (22.16 g, 156.11 mmol) was added to a solution of (2R,3S,4R)-2-(acetoxymethyl)-3,4-dihydro-2H-pyran-3,4-diyl diacetate (50 g, 183.65 mmol) in MeOH (12.95 g, 404.04 mmol, 16.35 mL, 2.2 eq.) and Tol. (600 mL) at 0° C. The mixture was stirred at 20° C. for 6.5 hrs. The reaction was clean according to TLC. The solution was cooled to 0° C., and quenched with Et₃N (20.45 mL), after maintaining for 10 min at 0° C., Na₂CO₃ (19.45 g) was added to the solution. The filtrate was concentrated to give a residue, which was purified by silica gel column chromatography (SiO₂, Petroleum ether/Ethyl acetate=100/1 to 1/1, with 1.0 vol % Et₃N). ((2R,3S)-3-acetoxy-6-methoxy-3,6-dihydro-2H-pyran-2-yl)methyl acetate (44 g, 98.09% yield) was obtained as a colorless oil. ¹H NMR (CHLOROFORM-d, 400 MHz): δ=5.73-5.87 (m, 2H), 5.25 (dd, J=9.7, 1.5 Hz, 1H), 4.86 (s, 1H), 4.13-4.25 (m, 2H), 3.93-4.03 (m, 1H), 3.39 (s, 3H), 2.04 (s, 3H), 2.02 ppm (s, 4H); TLC (Petroleum ether:Ethyl acetate=5:1) Rf=0.23.

Step 2. To a solution of ((2R,3S)-3-acetoxy-6-methoxy-3,6-dihydro-2H-pyran-2-yl)methyl acetate (44 g, 180.15 mmol) in EtOAc (400 mL) was added Pd/C (0.07 mg, 180.15 mmol, 10% purity). The mixture was stirred at 25° C. for 18 hrs under H₂ (15 Psi) atmosphere. The reaction was clean according to TLC. The insoluble material was removed by filtration through a pad of celite. The pad was washed with EtOAc (100 mL). The filtrate was concentrated. The residue was purified by column chromatography (SiO₂, Petroleum ether/Ethyl acetate=100/1 to 0/1). ((2R,3S)-3-acetoxy-6-methoxytetrahydro-2H-pyran-2-yl)methyl acetate (43 g, 96.93% yield) was obtained as a colorless oil. ¹H NMR (CHLOROFORM-d, 400 MHz): δ=4.67-4.80 (m, 2H), 4.22-4.31 (m, 1H), 4.11 (dd, J=11.9, 2.3 Hz, 1H), 3.90 (ddd, J=10.0, 5.3, 2.3 Hz, 1H), 3.37 (s, 3H), 2.09 (s, 3H), 2.04-2.06 (m, 3H), 1.81-2.02 ppm (m, 4H); TLC: (Petroleum ether:Ethyl acetate=5:1) Rf=0.30.

Step 3. For two batches: To a solution of ((2R,3S)-3-acetoxy-6-methoxytetrahydro-2H-pyran-2-yl)methyl acetate (54 g, 219.28 mmol) in AcOH (135 mL) was added Ac₂O (294.30 g, 2.88 mmol) and then added PTSA (75.52 g, 438.57 mmol). The mixture was stirred at 0° C. for 4 hr. TLC indicated compound 3 was consumed completely and one new spot formed. The reaction was into 500 mL of ice water, and the organic phase was separated, washed with water (5×300 mL), dried over Na₂SO₄, and evaporated under reduced pressure. And then the crude product to remove remaining acetic acid and dried in vacuum. The residue was purified by column chromatography (SiO₂, Petroleum ether:Ethyl acetate=15:0 to 0:1). (5S,6R)-6-(acetoxymethyl)tetrahydro-2H-pyran-2,5-diyl diacetate (109.6 g, 84.31% yield) was obtained as a colorless oil. ¹H NMR (400 MHz, DMSO-d6) δ=5.98 (br s, 1H), 4.68 (dt, J=4.8, 10.4 Hz, 1H), 4.21-4.10 (m, 1H), 4.06-3.89 (m, 2H), 2.07 (s, 3H), 2.01 (s, 3H), 2.00-1.99 (m, 3H), 1.97-1.93 (m, 1H), 1.89-1.70 (m, 3H); TLC: Petroleum ether:Ethyl acetate=1:1.5, Rf=0.39.

Step 4. To a solution of (5S,6R)-6-(acetoxymethyl)tetrahydro-2H-pyran-2,5-diyl diacetate (23 g, 83.86 mmol) in DCM (300 mL) was added hydrogen bromide (1 M) in N₂. The mixture was stirred at 0° C. for 4 hr. The reaction mixture poured into 300 mL of ice water, and the organic phase was separated, washed with water (5×200 mL), dried over MgSO₄, and evaporated under reduced pressure, and then the crude product to remove remaining acetic acid and dried in vacuum. ((2R,3S)-3-acetoxy-6-bromotetrahydro-2H-pyran-2-yl)methyl acetate (24 g, 96.97% yield) was obtained as a yellow oil. TLC: Petroleum ether:Ethyl acetate=1:1), Rf=0.26.

Step 5. For two batches: To a solution of 1-acetyl-114-pyrimidine-2,4(1H,3H)-dione (25.07 g, 162.64 mmol) in ACN (2000 mL) was added NaH (6.51 g, 162.64 mmol) for 0.5 hr at 0° C. and then added ((2R,3S)-3-acetoxy-6-bromotetrahydro-2H-pyran-2-yl)methyl aceta (12 g, 40.66 mmol) in N₂. The mixture was stirred at 0-60° C. for 3.5 hr. TLC indicated compound 5 was consumed completely and three new spots formed. The reaction mixture of two batches were filtered and concentrated under reduced pressure to give a residue. The crude product was purification by prep-HPLC (column: Welch Xtimate C18 250*70 mm #10 um; mobile phase: [water (NH₄HCO₃)-ACN]; B %: 10%-40%, 20 min). ((2R,3S,6R)-3-acetoxy-6-(2,6-dioxo-3,6-dihydropyrimidin-1(2H)-yl)tetrahydro-2H-pyran-2-yl)methyl acetate (1.5 g, 11.5% yield) was obtained as a white solid. ¹H NMR (400 MHz, DMSO-d6) δ=11.00 (br s, 1H), 7.42 (d, J=7.6 Hz, 1H), 5.92 (dd, J=1.9, 11.2 Hz, 1H), 5.55 (d, J=7.6 Hz, 1H), 4.59 (dt, J=4.6, 10.4 Hz, 1H), 4.11-3.99 (m, 2H), 3.75 (ddd, J=2.8, 5.1, 9.7 Hz, 1H), 2.99-2.85 (m, 1H), 2.16-2.08 (m, 1H), 2.00 (d, J=10.6 Hz, 6H), 1.74-1.61 (m, 2H); LCMS (M+H⁺): 349.1; TLC: Petroleum ether:Ethyl acetate=1:1, Rf=0.07.

Step 6. To a solution of ((2R,3S,6R)-3-acetoxy-6-(2,6-dioxo-3,6-dihydropyrimidin-1(2H)-yl)tetrahydro-2H-pyran-2-yl)methyl acetate (1.5 g, 4.60 mmol) in MeOH (50 mL) was added NaOMe (745.04 mg, 13.79 mmol). The mixture was stirred at 25° C. for 2 hr. TLC indicated compound 6 was consumed completely and one new spot formed. The reaction was added NH₄Cl (0.75 g) and stirred at room temperature for 5 min, and then vacuum concentration. The residue was purified by column chromatography (SiO₂, Petroleum ether:Ethyl acetate=10:1 to 0:1 to Ethyl acetate: MeOH=3:1). 3-((2R,5S,6R)-5-hydroxy-6-(hydroxymethyl)tetrahydro-2H-pyran-2-yl)pyrimidine-2,4(1H,3H)-dione (1 g, 89.81% yield) was obtained as a white solid. LCMS (M+H⁺): 507.1, purity: 92.82%; TLC: Ethyl acetate: Methanol=9:1, Rf=0.13.

Step 7. To a solution of 3-((2R,5S,6R)-5-hydroxy-6-(hydroxymethyl)tetrahydro-2H-pyran-2-yl)pyrimidine-2,4(1H,3H)-dione (0.8 g, 3.30 mmol) in Py (10 mL) was added DMTCl (1.12 g, 3.30 mmol) in N₂. The mixture was stirred at 25° C. for 6 hr. The reaction mixture was filtered and concentrated under reduced pressure to give a residue. The residue was purified by column chromatography (SiO₂, Petroleum ether:Ethyl acetate=10:1 to 0: 1 to DCM: MeOH=3:1, 5% TEA). Compound WV-NU-279 (0.8 g, 44.48% yield) was obtained as a yellow solid. ¹H NMR (400 MHz, DMSO-d6) δ=11.14-11.03 (m, 1H), 7.47-7.38 (m, 3H), 7.30-7.16 (m, 7H), 6.82 (br d, J=7.5 Hz, 4H), 5.87 (br d, J=10.8 Hz, 1H), 5.59 (d, J=7.6 Hz, 1H), 4.73 (d, J=6.1 Hz, 1H), 3.72 (s, 6H), 3.47-3.39 (m, 1H), 3.23 (br d, J=9.9 Hz, 1H), 2.99 (br dd, J=6.3, 9.9 Hz, 1H), 2.91-2.77 (m, 1H), 2.18 (t, J=8.0 Hz, 1H), 1.90 (quin, J=7.6 Hz, 1H), 1.58 (br d, J=11.1 Hz, 1H), 1.52-1.41 (m, 1H); LCMS (M−H+):543.3, purity: 91.91%; TLC: Dichloromethane: Methanol=9:1, Rf=0.42.

### Synthesis of 3-((2R,5S,6R)-6-((bis(4-methoxyphenyl)(phenyl)methoxy)methyl)-5-(((1S,3S,3aS)-3-((phenylsulfonyl)methyl)tetrahydro-1H,3H-pyrrolo [1,2-c][1,3,2]oxazaphosphol-1-yl)oxy)tetrahydro-2H-pyran-2-yl)pyrimidine-2,4(1H,3H)-dione

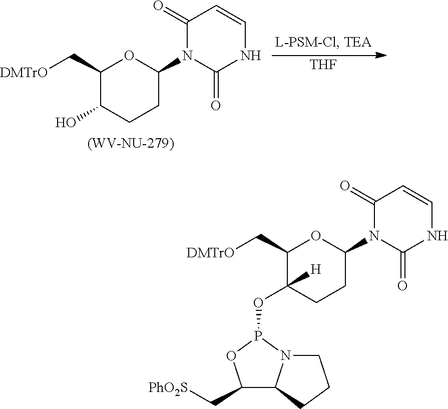

Amidite 9 (N568-374) was synthesized using general procedure from WV-NU-279. Yield, 38%. ³¹P NMR (243 MHz, CDCl₃) δ 150.41; MS (ES) m/z calculated for C₄₃H₄₆N₃O₁₀PS [M+K]⁺ 866.23, Observed: 866.19 [M+K]⁺.

### Synthesis of stereorandom C12 linker tri-antennary GalNAc phosphoramidite

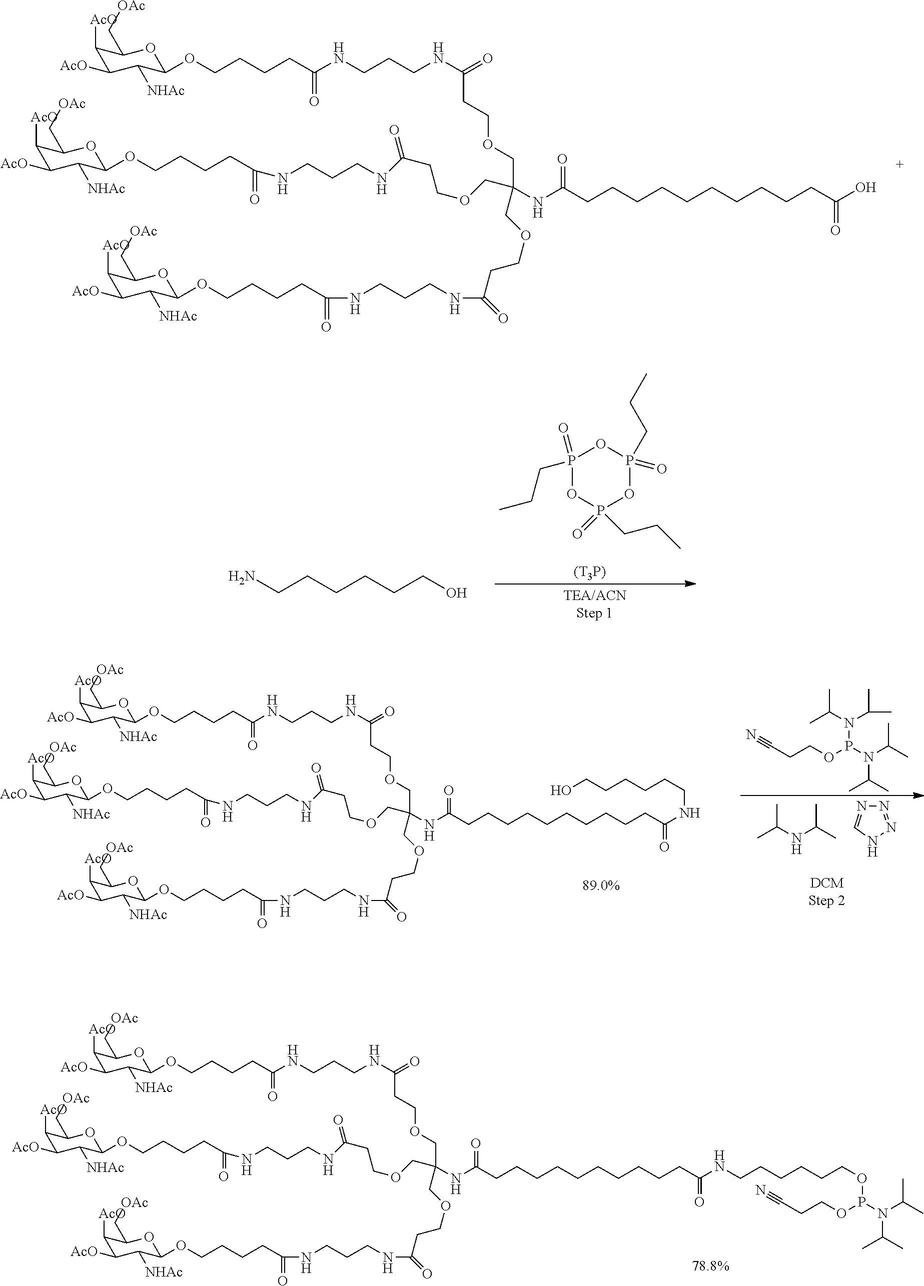

Step 1. To a solution of 6-Amino-1-hexanol (8.76 g, 74.8 mmol) in ACN (350 mL) was added GalNAc acetyl derivative (50.0 g, 24.9 mmol) followed by triethylamine (10.4 mL, 74.8 mmol). T3P (31.7 g, 49.8 mmol) (50 wt % in ACN) was added slowly over a period of 10 min. Under argon balloon protection, the r×n solution was stirred at rt for 21 hr. TLC and LCMS showed the r×n was complete. The r×n mixture was filtered through celite, and the filtrate was concentrated. The resulting crude product was redissolved in DCM (375 ml) and washed with water (95 mL). The aqueous layer including the thick brown layer between the organic layer and the aqueous layer was back-extracted with DCM (4×375 ml). The thick brown layer was released to the organic layer. The resulting cloudy organics were dried over anhydrous sodium sulfate and stirred for 30 min. The organics became a clear solution. The organics were filtered through a plastic filter funnel. The filtrate was concentrated to afford the crude product as a foam, which was purified by normal phase column chromatography (Silica RediSepRf Gold 330 g HP Column) applying 0-50% MeOH in DCM as the gradient to afford [(3R,5S,6R)-5-acetamido-6-[5-[3-[3-[3-[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]methyl]-2-[[12-(6-hydroxyhexylamino)-12-oxo-dodecanoyl]amino]propoxy]propanoylamino]propylamino]-5-oxo-pentoxy]-3,4-diacetoxy-tetrahydropyran-2-yl]methyl acetate as a foam (46.7 g, 89.0% yield). ¹H NMR (600 MHz, Chloroform-d) δ 7.30 (t, J=6.3 Hz, 3H), 7.08 (t, J=6.1 Hz, 3H), 6.95-6.87 (m, 3H), 6.60 (d, J=6.7 Hz, 1H), 6.04 (t, J=5.8 Hz, 1H), 5.34 (dd, J=3.5, 1.1 Hz, 3H), 5.18 (dt, J=11.2, 3.9 Hz, 3H), 4.61 (dd, J=8.3, 4.2 Hz, 3H), 4.16 (dd, J=11.2, 6.4 Hz, 3H), 4.13-4.04 (m, 6H), 3.96-3.87 (m, 6H), 3.69-3.65 (m, 11H), 3.61 (t, J -6.6 Hz, 3H), 3.49 (ddd, J -9.7, 7.4, 4.8 Hz, 3H), 3.34-3.20 (m, 14H), 2.43 (t, J=5.7 Hz, 6H), 2.30-2.16 (m, 8H), 2.14 (s, 9H), 2.04 (s, 9H), 1.98 (s, 9H), 1.94 (s, 9H), 1.74 (dt, J=14.2, 7.2 Hz, 3H), 1.70-1.47 (m, 25H), 1.41-1.32 (m, 5H), 1.29-1.22 (m, 11H). MS (ESI), 2105.21 [M+H]⁺.

Step 2. [(3R,5S,6R)-5-acetamido-6-[5-[3-[3-[3-[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]-2- [[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]methyl]-2-[[12-(6-hydroxyhexylamino)-12-oxo-dodecanoyl]amino]propoxy]propanoylamino]propylamino]-5-oxo-pentoxy]-3,4-diacetoxy-tetrahydropyran-2-yl]methyl acetate (46.7 g, 22.2 mmol) was co-evaporated at 35° C. to dryness first with anhydrous toluene (2×234 mL), then with 1:1 ratio of anhydrous toluene/anhydrous ACN (2×234 mL). and finally with anhydrous ACN (4×234 mL). The resulting foam was further dried on high vacuum overnight before the r×n. Under argon balloon protection, to a solution of the dry GalNAc alcohol derivative (46.7 g, 22.2 mmol) in DCM (130 mL) was added 2-cyanoethyl N,N,N,N-tetraisopropylphosphorodiamidite (14.1 mL, 44.4 mmol) slowly over a period of 5 min. Diisopropylammonium tetrazolide (1.52 g, 8.87 mmol) was added in one portion. The resulting r×n solution was stirred at rt for 4 hr. TLC and LCMS showed the r×n was complete. ³¹P NMR showed the desired product was formed and major. The r×n solution was concentrated at rt. The resulting crude product was purified by normal phase column chromatography (Silica RediSepRf Gold 330 g HP Column) by applying 0-100% ACN in EtOAc (mobile phase A contained 1% TEA; mobile phase B contained 1-5% TEA) as the gradient to afford the title compound as a foam (39.8 g, 77.8% yield). ¹H NMR (600 MHz, Chloroform-d) δ 7.25 (t, J=6.1 Hz, 3H), 7.01 (t, J=6.2 Hz, 3H), 6.86-6.77 (m, 3H), 6.56 (d, J=9.7 Hz, 1H), 5.82 (t, J=5.7 Hz, 1H), 5.38-5.32 (m, 3H), 5.22-5.15 (m, 3H), 4.61 (dd, J=8.3, 4.5 Hz, 3H), 4.16 (dd, J=11.3, 6.5 Hz, 3H), 4.14-4.04 (m, 6H), 3.96-3.88 (m, 6H), 3.87-3.83 (m, 1H), 3.78 (ddt, J=10.2, 8.0, 6.3 Hz, 1H), 3.69-3.65 (m, 11H), 3.58 (ddt, J=14.3, 7.6, 3.6 Hz, 3H), 3.50 (ddd, J=9.7, 7.4, 4.8 Hz, 3H), 3.33-3.19 (m, 14H), 2.64 (t, J=6.4 Hz, 2H), 2.46-2.40 (m, 6H), 2.29-2.16 (m, 8H), 2.14 (s, 9H), 2.04 (s, 9H), 1.99 (s, 9H), 1.94 (s, 9H), 1.75 (dt, J=14.2, 7.2 Hz, 3H), 1.70-1.47 (m, 25H), 1.42-1.31 (m, 5H), 1.30-1.22 (m, 13H), 1.17 (dd, J=6.8, 5.7 Hz, 12H). “P NMR (243 MHz, Chloroform-d) δ 147.23. MS (ESI), 2343.06 [M +K]⁺.

Preparation for additional compounds useful for oligonucleotide preparation were described below, in some embodiments following preparations described in WO 2022/099159:

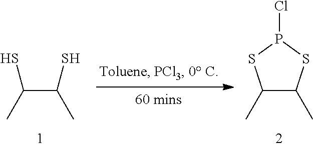

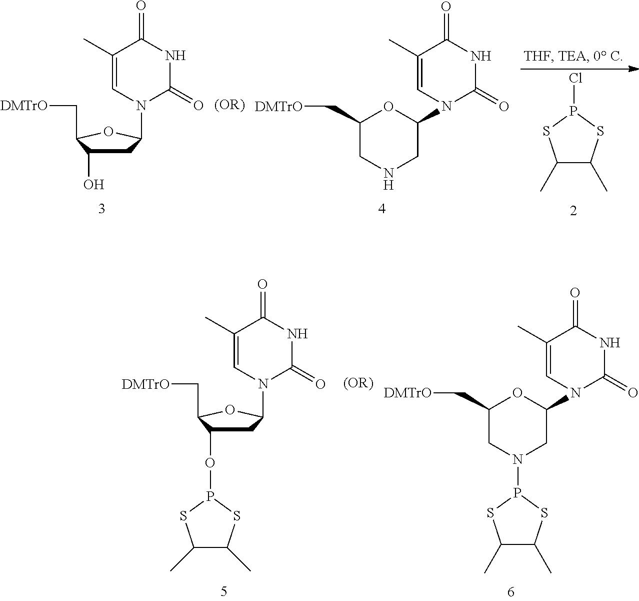

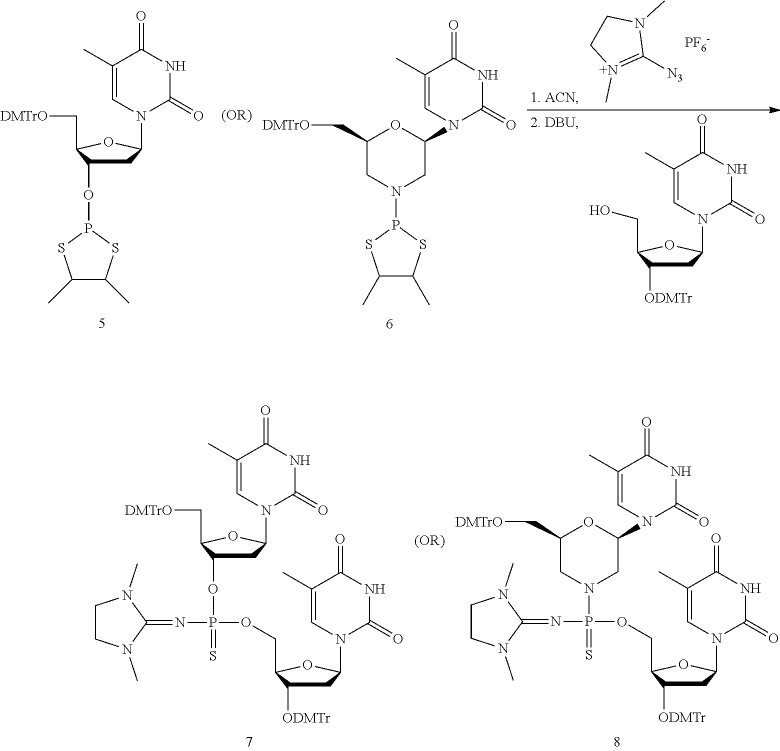

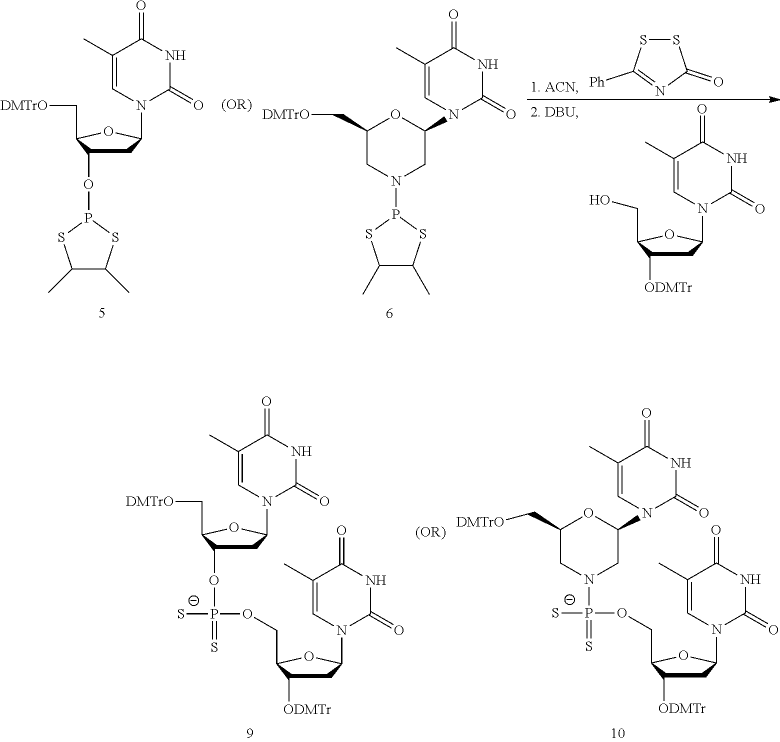

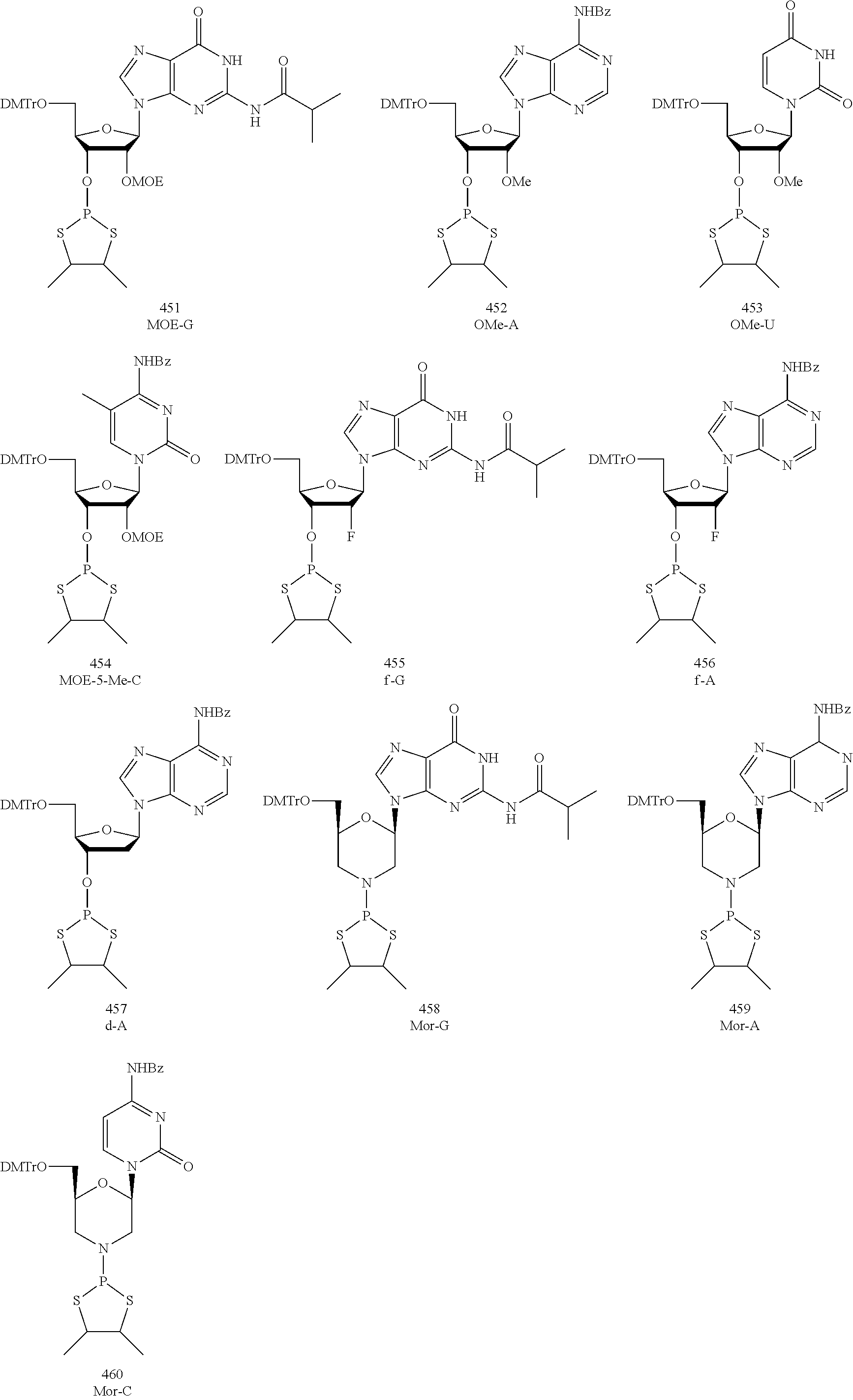

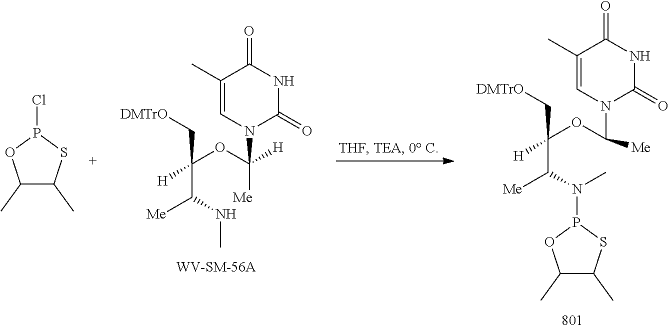

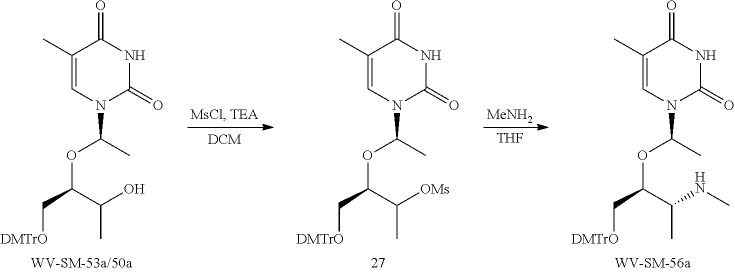

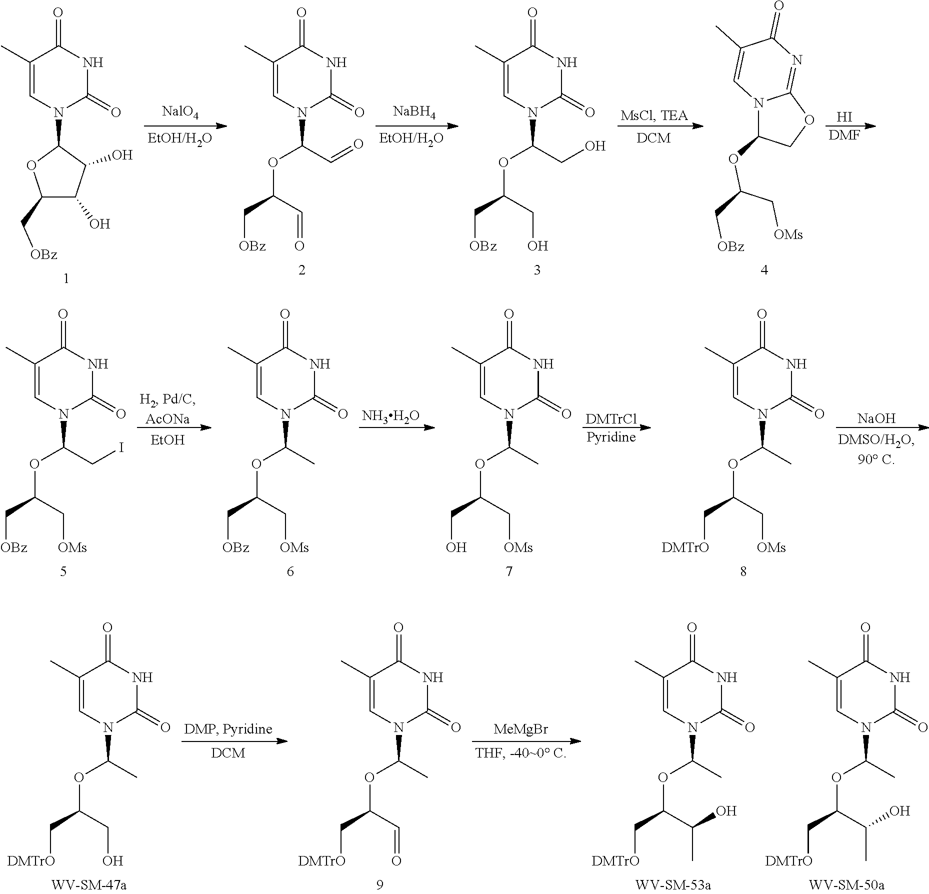

### Synthesis of 3-((2R,3R,4R,5R)-5-((bis(4-methoxyphenyl)(phenyl)methoxy)methyl)-4-hydroxy-3-(2-methoxyethoxy)tetrahydrofuran-2-yl)pyrimidine-2,4(1H,3H)-dione (WV-NU-300) and 3-((2R,3R,4S,5R)-5-((bis(4-methoxyphenyl)(phenyl)methoxy)methyl)-3-hydroxy-4-(2-methoxyethoxy)tetrahydrofuran-2-yl)pyrimidine-2,4(1H,3H)-dione (WV-NU-300A)

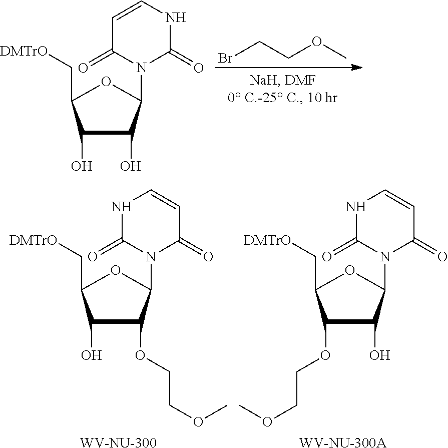

For 4 batches: To a solution of 3-((2R,3R,4S,5R)-5-((bis(4-methoxyphenyl)(phenyl)methoxy)methyl)-3,4-dihydroxytetrahydrofuran-2-yl)pyrimidine-2,4(1H,3H)-dione (5 g, 9.15 mmol) in DMF (50 mL) and NaH (475.65 mg, 11.89 mmol, 60% purity) was added at 0° C. for 0.5 hr under N₂ and the then 1-bromo-2-methoxy-ethane (1.27 g, 9.15 mmol) was dropped and mixture was stirred at 20° C. for 12 hr. The water (25 mL) was dropped to the mixture at 0° C. under N₂ and extracted with EtOAc (50 mL*3), the combined organic was dried over Na₂SO₄, filtered and concentrated to get the crude. The mixture was purified by prep-HPLC column: Waters Xbridge BEH C18 250*50 mm*10 um; mobile phase: [water (NH₄HCO₃)-ACN]; B %: 25%-55%, 10 min] to get WV-NU-300 (1.9 g, 8.52% yield) as a yellow solid and WV-NU-300A (7 g, crude) was a yellow solid, the WV-NU-300A was further purification by MPLC: column: Welch Xtimate C18 250*70 mm #10 um; mobile phase: [water (NH₄HCO₃)-ACN]; B %: 40%-70%, 22 min to get (4 g, 16% yield) as a white solid. WV-NU-300: ¹HNMR (400 MHz, DMSO-d6) δ=11.27-11.04 (m, 1H), 7.51-7.47 (m, 1H), 7.42-7.38 (m, 2H), 7.31-7.24 (m, 6H), 7.23-7.18 (m, 1H), 6.86 (dd, J=5.9, 8.9 Hz, 4H), 6.17-6.14 (m, 1H), 5.63-5.58 (m, 1H), 4.77-4.72 (m, 1H), 4.30-4.22 (m, 2H), 3.89-3.82 (m, 1H), 3.76-3.72 (m, 6H), 3.72-3.65 (m, 1H), 3.59-3.52 (m, 1H), 3.47-3.40 (m, 2H), 3.23 (s, 3H), 3.18-3.12 (m, 1H), 3.11-3.03 (m, 1H); LCMS: (M−H+): 603.2, LCMS purity: 99.22%. WV-NU-300A: ¹HNMR (400 MHz, DMSO-d6) δ=7.64 (d, J=7.9 Hz, 1H), 7.40 (d, J=7.7 Hz, 2H), 7.29-7.16 (m, 7H), 6.89-6.80 (m, 4H), 6.14 (d, J=2.3 Hz, 1H), 5.67 (d, J=7.8 Hz, 1H), 5.10 (br d, J=4.4 Hz, 1H), 4.86 (br d, J=7.5 Hz, 1H), 4.32 (br d, J=2.0 Hz, 1H), 4.19 (q, J=7.1 Hz, 1H), 3.91-3.81 (m, 3H), 3.72 (s, 6H), 3.46 (br s, 2H), 3.20 (s, 3H), 3.17-3.03 (m, 2H); LCMS: (M−H+): 603.2, LCMS purity: 100.00%.

### Synthesis of 3-((2R,5S,6R)-6-((bis(4-methoxyphenyl)(phenyl)methoxy)methyl)-5-hydroxytetrahydro-2H-pyran-2-yl)pyrimidine-2,4(1H,3H)-dione (WV-NU-279) and 3-((2S,5S,6R)-6-((bis(4-methoxyphenyl)(phenyl)methoxy)methyl)-5-hydroxytetrahydro-2H-pyran-2-yl)pyrimidine-2,4(1H,3H)-dione (WV-NU-279A)

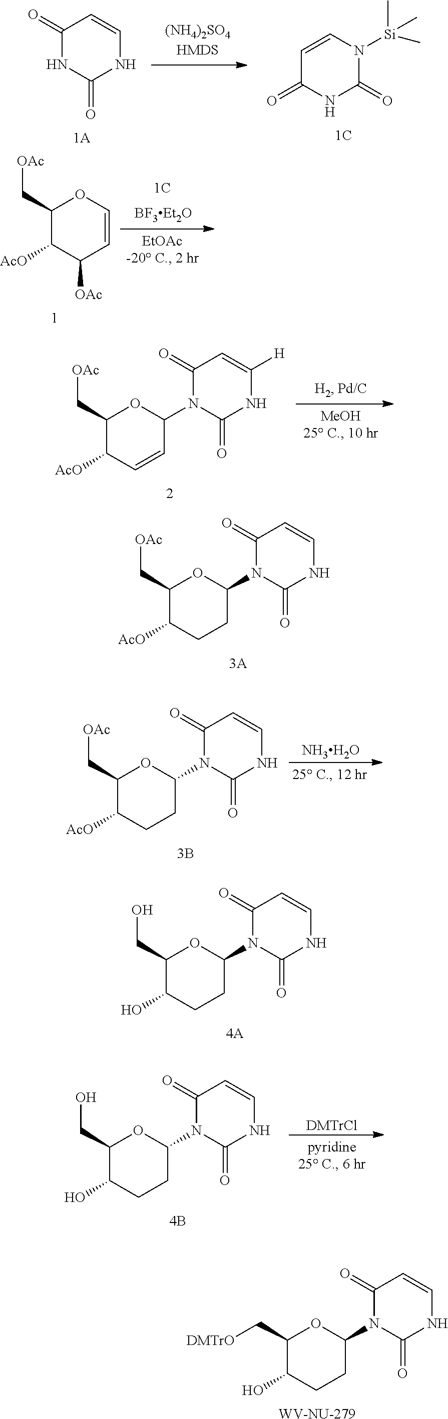

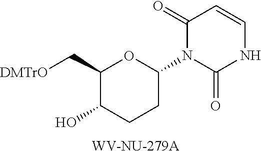

Step 1. For four batches: To a solution of pyrimidine-2,4(1H,3H)-dione (25 g, 223.04 mmol) in HMDS (192.50 g, 1.19 mol) was added (NH₄)2SO₄ (5.89 g, 44.61 mmol) under N₂. The mixture was stirred at 140° C. for 2 hr. The reaction mixture was filtered and concentrated under reduced pressure to give a compound 1C (102.6 g, 92.60% yield) as a yellow oil.

Step 2. For two batches: To a solution of compound 1 (50 g, 183.65 mmol) in EtOAc (1000 mL) at −20° C. was added compound 1C (51.32 g, 275.48 mmol) and then added BF₃.Et₂O (338.86 g, 2.39 mol) under N₂. The mixture was stirred at −20° C. for 2 hr. TLC indicated compound 1 was consumed completely and one new spot formed. The reaction mixture was quenched by addition sat. NaHCO₃ 500 mL at 0° C., and then extracted with EtOAc (200 mL*3). The combined organic layers were dried over Na₂SO₄, filtered and concentrated under reduced pressure to give a residue to get compound 2 (78 g, crude) as a white solid. ¹H NMR (400 MHz, DMSO-d6) δ=11.23-11.02 (m, 1H), 7.44 (br s, 1H), 6.61-6.39 (m, 1H), 5.99-5.90 (m, 1H), 5.89-5.69 (m, 1H), 5.58 (br d, J=7.5 Hz, 1H), 5.28-5.13 (m, 1H), 4.15-3.99 (m, 3H), 2.09-1.97 (m, 6H); LCMS: (M+H+): 325.1.

Step 3. To a mixture solution of compound 2 (78 g, 240.53 mmol) in MeOH (1000 mL) was added Pd/C (10 g, 10% purity) under H₂ (485.87 mg, 240.53 mmol) 15 psi. The mixture was stirred at 25° C. for 10 hr. LCMS showed the desired mass was detected. The reaction mixture was filtered and concentrated under reduced pressure to give a residue. The crude product was purification by prep-HPLC: column: Welch Xtimate C18 180*70 mm #10 um; mobile phase: [water (10 mM NH₄HCO₃)-ACN]; gradient: 0%-25% B over 17.0 min. Compound 3A (18 g, 26.47% yield) was obtained as a white solid. Compound 3B (18 g, 26.47% yield) was obtained as a white solid. Compound 3A: ¹HNMR (400 MHz, DMSO-d6) 6-11.10 (br s, 1H), 7.42 (d, J -7.6 Hz, 1H), 5.92 (dd, J=1.9, 11.1 Hz, 1H), 5.55 (d, J=7.6 Hz, 1H), 4.59 (dt, J=4.6, 10.3 Hz, 1H), 4.12-3.97 (m, 2H), 3.75 (ddd, J=2.8, 5.1, 9.8 Hz, 1H), 3.00-2.84 (m, 1H), 2.16-2.08 (m, 1H), 2.03-1.98 (m, 6H), 1.74-1.60 (m, 2H). Compound 3B: ¹HNMR (400 MHz, DMSO-d6) δ=11.10 (br s, 1H), 7.41 (d, J=7.5 Hz, 1H), 6.15 (dd, J=4.6, 10.8 Hz, 1H), 5.56 (d, J=7.6 Hz, 1H), 4.82-4.75 (m, 1H), 4.47-4.40 (m, 1H), 4.16-4.09 (m, 2H), 2.89-2.75 (m, 1H), 2.04 (s, 3H), 1.99 (s, 3H), 1.90-1.80 (m, 2H), 1.61-1.53 (m, 1H); LCMS: (M+H+): 349.1.

Step 4. To a solution of compound 3A (18 g, 55.16 mmol) in ammonia (7 M, 180 mL) under N₂. The mixture was stirred at 25° C. for 12 hr. TLC indicated compound 3A was consumed completely and one new spot formed. The reaction mixture was filtered and concentrated under reduced pressure to give Compound 4A (13 g, 97.29% yield) as a colorless oil. ¹H NMR (400 MHz, DMSO-d6) 6=7.39 (d, J=7.5 Hz, 1H), 5.78 (dd, J=2.0, 11.1 Hz, 1H), 5.52 (d, J=7.6 Hz, 1H), 3.68 (dd, J=1.6, 11.7 Hz, 1H), 3.37 (br dd, J=6.3, 11.7 Hz, 3H), 3.22-3.15 (m, 3H), 2.89-2.78 (m, 1H), 2.04-1.97 (m, 1H), 1.56-1.37 (m, 2H); LCMS: (M+H+): 243.1, purity: 97.53%.

Step 4b. To a solution of compound 3B (18.00 g, 55.16 mmol) in ammonia (7 M, 180 mL) under N₂. The mixture was stirred at 25° C. for 12 hr. TLC indicated compound 3B was consumed completely and one new spot formed. The reaction mixture was filtered and concentrated under reduced pressure to give a residue. Compound 4B (13 g, 97.29% yield) was obtained as a colorless oil. ¹H NMR (400 MHz, DMSO-d6) δ=7.39 (d, J=7.6 Hz, 1H), 6.07 (dd, J=4.6, 10.8 Hz, 1H), 5.52 (d, J=7.5 Hz, 1H), 4.01-3.94 (m, 1H), 3.67-3.43 (m, 5H), 2.83 (dq, J=5.2, 11.9 Hz, 1H), 1.83-1.66 (m, 2H), 1.48-1.39 (m, 1H); LCMS: (M+H⁺): 243.1.

Step 5. To a solution of compound 4A (13 g, 53.67 mmol) in pyridine (200 mL) was added DMT-Cl (18.18 g, 53.67 mmol) under N₂. The mixture was stirred at 15° C. for 6 hr. TLC indicated compound 4A was consumed completely and two new spot formed. The reaction mixture was quenched by addition MeOH 20 mL, and then filtered and concentrated under reduced pressure to give a residue. The residue was purified by column chromatography (SiO₂, Petroleum ether:Ethyl acetate=20:1 to 0:1 to Ethyl acetate: Methanol=5:1, 5% TEA). Compound WV-NU-279 (21 g, 72.41% yield) was obtained as a white solid. ¹H NMR (400 MHz, DMSO-d6) δ=11.11 (br s, 1H), 7.48-7.38 (m, 3H), 7.31-7.15 (m, 7H), 6.83 (dd, J=1.8, 8.8 Hz, 4H), 5.87 (br d, J=10.0 Hz, 1H), 5.59 (d, J=7.6 Hz, 1H), 4.73 (d, J=6.1 Hz, 1H), 3.72 (s, 6H), 3.47-3.39 (m, 1H), 3.32-3.20 (m, 2H), 2.99 (dd, J=6.2, 9.9 Hz, 1H), 2.90-2.79 (m, 1H), 2.06-2.00 (m, 1H), 1.58 (br d. J =11.3 Hz, 1H), 1.52-1.41 (m, 1H); LCMS: (M−H⁺): 543.1, purity: 98.8%.

Step 5b. To a solution of compound 4B (13 g, 53.67 mmol) in pyridine (200 mL) was added DMT-C1 (18.18 g, 53.67 mmol) under N₂. The mixture was stirred at 15° C. for 6 hr. TLC indicated compound 4B was consumed completely and two new spots formed. The reaction mixture was quenched by addition MeOH 20 mL, and then filtered and concentrated under reduced pressure to give a residue. The residue was purified by column chromatography (SiO₂, Petroleum ether:Ethyl acetate=20:1 to 0:1 to Ethyl acetate: Methanol=5:1, 5% TEA). Compound WV-NU-279A (8.8 g, 30.34% yield) was obtained as a white solid and 8 g crude need further purification. ¹HNMR (400 MHz, DMSO-d6) δ=11.09 (s, 1H), 7.47-7.35 (m, 3H), 7.33-7.16 (m, 7H), 6.91-6.82 (m, 4H), 6.11 (dd, J=4.8, 10.6 Hz, 1H), 5.58 (d, J=7.5 Hz, 1H), 4.73 (d, J=5.4 Hz, 1H), 4.31-4.24 (m, 1H), 3.72 (s, 6H), 3.58-3.51 (m, 1H), 3.15-3.08 (m, 1H), 3.07-2.99 (m, 1H), 2.94-2.81 (m, 1H), 1.78-1.67 (m, 2H), 1.54-1.44 (m, 1H); LCMS: (M−H⁺): 543.2, LCMS purity: 94.43%.

### Synthesis of N-(7-benzoyl-9-((2R,3R,4R,5R)-5-((bis(4-methoxyphenyl)(phenyl)methoxy)methyl)-4-hydroxy-3-methoxytetrahydrofuran-2-yl)-8-oxo-8,9-dihydro-7H-purin-6-yl)benzamide (WV-NU-312A)

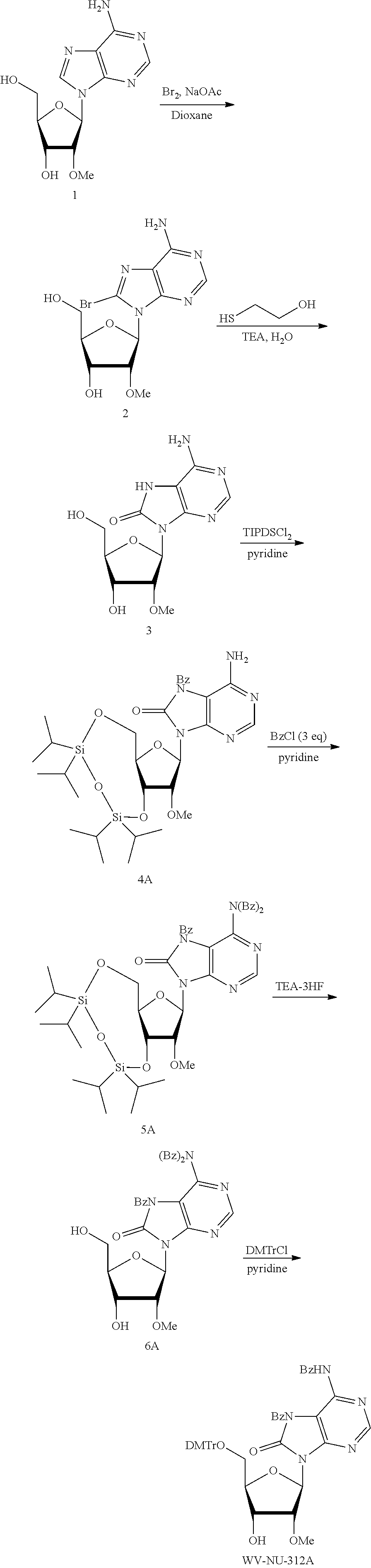

Step 1. A mixture of compound 1 (10 g, 35.55 mmol), Br₂ (11.36 g, 71.11 mmol), NaOAc (0.5 M, 142.92 mL, HOAc buffer pH=4.7) in dioxane (300 mL) was degassed and purged with N₂ for 3 times, and then the mixture was stirred at 20° C. for 10 hr under N₂ atmosphere. LCMS showed compound 1 was consumed completely and one main peak with desired mass was detected. The reaction mixture was quenched by addition Na₂S₂O₅ until the red color disappear, and then the mixture was added 0.5M NaOH until pH=7 and then the mixture was concentrated under reduced pressure and concentrated under reduced pressure to give compound 2 (12.8 g, crude) was obtained as a white solid. ¹H NMR (400 MHz, DMSO-d6) δ −8.13 (s, 1H), 7.78-7.41 (m, 2H), 5.92 (d, J=6.8 Hz, 1H), 5.50 (dd, J=4.0, 8.3 Hz, 1H), 5.36 (d, J=5.3 Hz, 1H), 4.89-4.81 (m, 1H), 4.46-4.36 (m, 1H), 4.04-3.96 (m, 1H), 3.68 (td, J=4.0, 12.2 Hz, 1H), 3.55-3.49 (m, 1H), 3.28 (s, 3H); LCMS (M+H⁺): 362.0.

Step 2. To a solution of compound 2 (12.8 g, 35.54 mmol) in water (130 mL) was added TEA (35.96 g, 355.39 mmol) and 2-sulfanylethanol (13.26 g, 169.69 mmol). The mixture was stirred at 100° C. for 10 hr. LCMS showed compound 2 remained. Several new peaks were shown on LCMS and desired compound was detected. The reaction mixture was combined and concentrated under reduced pressure to remove H2O, and then the mixture was purified by RP-MPLC (NH4HCO3, H2O/MeCN) to get compound 3 (8 g, 80%) was obtained as a yellow solid. ¹H NMR (400 MHz, DMSO-d6) δ=10.45-10.33 (m, 1H), 8.10-7.98 (m, 1H), 6.66-6.55 (m, 2H), 5.83-5.73 (m, 1H), 5.24-5.14 (m, 2H), 4.69-4.53 (m, 1H), 4.37-4.29 (m, 1H), 3.93-3.85 (m, 1H), 3.64-3.58 (m, 1H), 3.53-3.44 (m, 1H), 3.30-3.27 (m, 3H); LCMS: (M+H⁺): 298.1.

Step 3. To a solution of compound 3 (10 g, 33.64 mmol) in PYRIDINE (100 mL) and chloro-[chloro(diisopropyl)silyl]oxy-diisopropyl-silane (15.92 g, 50.46 mmol) was added and the mixture was stirred at 15° C. for 1 hr. LCMS showed the compound 3 was consumed and the desired mass was obtained. The mixture was concentrated to get the crude. The residue was purified by MPLC (Petroleum ether:Ethyl acetate =10:1, 5:1, 1:1) to get compound 4A (15 g, 82.61% yield) as a yellow oil. ¹H NMR (400 MHz, CHLOROFORM-d) δ=10.58 (br s, 1H), 8.11 (s, 1H), 5.79 (d, J=1.1 Hz, 1H), 5.66 (br s, 2H), 5.25 (dd, J=5.4, 8.9 Hz, 1H), 4.58 (d, J=5.5 Hz, 1H), 4.09-3.93 (m, 4H), 3.60 (s, 3H), 1.12-1.02 (m, 26H); LCMS: (M+H⁺): 540.3.

Step 4. To a solution of compound 4A (13 g, 24.08 mmol) in PYRIDINE (200 mL) and BzCl (11.85 g, 84.30 mmol) was added and the mixture was stirred at 15° C. for 12 hr. LCMS showed the compound 4A was consumed and the desired mass was found. The reaction mixture was concentrated under reduced pressure to remove pyridine. The residue was purified by column chromatography (SiO₂, Petroleum ether/Ethyl acetate=1/0 to 0/1) to get compound 5A (13 g, 63.35% yield) as a yellow solid. ¹H NMR (400 MHz, CHLOROFORM-d) 6=8.31 (s, 1H), 7.99 (br d, J=7.4 Hz, 2H), 7.94 (br d, J=7.5 Hz, 2H), 7.87 (d, J=7.3 Hz, 2H), 7.57-7.45 (m, 3H), 7.42-7.33 (m. 6H), 5.91 (d, J=1.1 Hz, 1H), 5.12 (dd, J=5.5, 9.3 Hz, 1H), 4.47 (d. J=5.5 Hz, 1H), 4.15-4.05 (m, 1H), 4.03-3.90 (m, 2H), 3.82-3.75 (m, 1H), 3.68-3.62 (m, 1H), 3.60 (s, 3H), 1.11-1.03 (m, 18H), 1.00-0.92 (m, 8H); LCMS: (M+H+): 853.6.

Step 5. To a solution of compound 5A (13 g, 15.26 mmol) in THF (130 mL) was added N,N-diethylethanamine;trihydrofluoride (7.38 g, 45.77 mmol). The mixture was stirred at 25° C. for 2 hr. LCMS showed the compound 5A was consumed completely and desired mass was detected. The reaction mixture was concentrated under reduced pressure to remove THF. The residue was purified by column chromatography (SiO₂, Petroleum ether/Ethyl acetate=1/0 to 0/1) to get compound 6A (9 g, 96.77% yield) as a white solid. ¹H NMR (400 MHz, DMSO-d6) δ=8.54 (s, 1H), 7.89 (br s, 2H), 7.84 (br d, J=7.4 Hz, 3H), 7.65-7.58 (m, 1H), 7.58-7.53 (m, 2H), 7.51-7.40 (m, 7H), 5.82 (d, J=4.3 Hz, 1H), 5.17 (d, J=6.5 Hz, 1H), 4.72 (t, J=5.9 Hz, 1H), 4.57 (t, J=4.9 Hz, 1H), 4.37 (q, J=5.7 Hz, 1H), 3.87-3.78 (m, 1H), 3.61 (td, J=4.6, 11.7 Hz, 1H), 3.45 (td, J=6.2, 12.0 Hz, 1H), 3.40-3.36 (m, 3H); LCMS: (M+H+): 610.1.

Step 6. To a solution of compound 6A (9 g, 14.76 mmol) in PYRIDINE (100 mL) and DMTCl (5.00 g, 14.76 mmol) was added and the mixture was stirred at 15° C. for 1 hr, TLC (Petroleum ether:Ethyl acetate=1:2, Rf=0.63) showed the compound 6A was consumed and a new spot was found. MeOH (10 mL) was added and stirred for 10 min and the mixture was concentrated to get the crude. The residue was purified by MPLC (Petroleum ether:Ethyl acetate=10:1, 5:1, 1:1) to get WV-NU-312A (9 g, 75.46% yield) as a white solid. ¹HNMR (400 MHz, DMSO-d6) δ=8.40 (s, 1H), 7.89 (br s, 3H), 7.83 (br d, J=7.4 Hz, 2H), 7.64-7.57 (m, 1H), 7.54 (br s, 2H), 7.45-7.38 (m, 6H), 7.35 (br d, J=7.2 Hz, 2H), 7.24-7.10 (m, 7H), 6.80 (dd, J=2.9, 8.9 Hz, 4H), 5.85 (d, J=3.0 Hz, 1H), 5.17 (d, J=6.8 Hz, 1H), 4.54-4.42 (m, 2H), 4.00-3.96 (m, 1H), 3.70 (s, 6H), 3.39 (s, 3H), 3.17 (br d, J=4.2 Hz, 2H); LCMS: (M−H+): 806.2, LCMS purity: 92.46%.

### Synthesis of 3-((2R,3R,4R,5R)-5-((bis(4-methoxyphenyl)(phenyl)methoxy)methyl)-3-(2-methoxyethoxy)-4-(((1S,3S,3aS)-3-((phenylsulfinyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

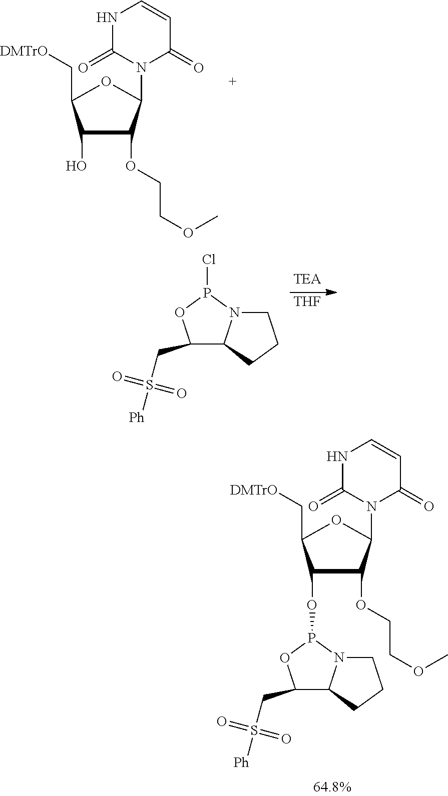

To a solution of dry 3-[(2R,3S,5R)-5-[[bis(4-methoxyphenyl)-phenyl-methoxy]methyl]-4-hydroxy-3-(2-methoxyethoxy)tetrahydrofuran-2-yl]-1H-pyrimidine-2,4-dione (1.83 g, 3.03 mmol) in THF (14 mL) was added triethylamine (0.97 mL, 6.96 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.9M soln in THF, 5.38 mL, 4.84 mmol) was added dropwise over a period of 5 min. The ice/water bath was removed. The resulting brownish r×n slurry was stirred at rt for 1.5 hr. TLC and LCMS showed that the r×n was complete. The reaction was quenched by water (27 μL). Anhydrous MgSO₄ (360 mg) was added. The mixture was filtered through celite, and the filtrate was concentrated. The resulting crude product was purified by normal phase column chromatography applying 30-100% EtOAc in hexanes (each mobile phase contained 2.5% triethylamine) as the gradient to afford the title compound as a brownish off-white foam (1.74 g, 64.8% yield).

¹H NMR (600 MHz, Chloroform-d) δ 7.90-7.86 (m, 2H), 7.61-7.57 (m, 1H), 7.51-7.47 (m, 2H), 7.47-7.44 (m, 2H), 7.36-7.31 (m, 4H), 7.23 (dd, J=8.4, 7.0 Hz, 2H), 7.18-7.12 (m, 1H), 6.81 (d, J=7.7 Hz, 1H), 6.80-6.75 (m, 4H), 6.34 (d, J=3.1 Hz, 1H), 5.55 (d, J=7.7 Hz, 1H), 5.12-5.06 (m, 1H), 4.85-4.78 (m, 1H), 4.55 (dd, J=6.1, 3.3 Hz, 1H), 4.10 (dt, J=6.7, 3.2 Hz, 1H), 3.79-3.76 (m, 1H), 3.744 (s, 3H), 3.742 (s, 3H), 3.66 (ddt, J=9.8, 5.8, 2.9 Hz, 2H), 3.59-3.50 (m, 2H), 3.42-3.34 (m, 3H), 3.33 (s, 3H), 3.34-3.30 (m, 1H), 3.19 (dd, J=10.4, 6.0 Hz, 1H), 2.87 (qd, J=9.7, 4.2 Hz, 1H), 1.80 (dtt, J=12.2, 7.9, 3.7 Hz, 1H), 1.74-1.65 (m, 1H), 1.61-1.53 (m, 1H), 1.11-1.05 (m, 1H); ³¹P NMR (243 MHz, Chloroform-d) δ 148.84; MS (ESI), 886.68 [M−H]⁻.

### Synthesis of N-(7-benzoyl-9-((2R,3R,4R,5R)-5-((bis(4-methoxyphenyl)(phenyl)methoxy)methyl)-3-methoxy-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)benzamide

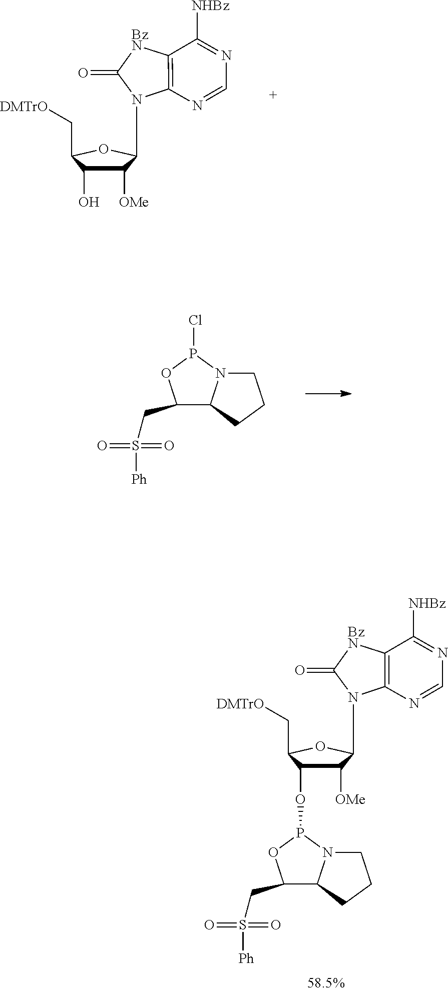

To a solution of dry N-[7-benzoyl-9-[(2R,3S,5R)-5-[[bis(4-methoxyphenyl)-phenyl-methoxy]methyl]-4-hydroxy-3-methoxy-tetrahydrofuran-2-yl]-8-oxo-purin-6-yl]benzamide (2.50 g, 3.09 mmol) in THF (15 mL) was added triethylamine (1.29 mL, 9.28 mmol). The r×n flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.746M soln in THF, 5.81 mL, 4.33 mmol) was added dropwise over a period of 5 min. The ice/water bath was removed. The resulting slurry was stirred at rt for 1.5 hr. TLC and LCMS showed that the r×n was complete. The reaction was quenched with water (28 L). Anhydrous MgSO₄ (372 mg) was added. The mixture was filtered through celite, and the filtrate was concentrated. The resulting crude product was purified by normal phase column chromatography applying 30-100% EtOAc in hexanes (each mobile phase contained 1% triethylamine) as the gradient to afford the title compound as a white foam (1.97 g, 58.5% yield). ¹H NMR (600 MHz, Acetonitrile-d3) δ 8.20 (s, 1H), 7.91-7.88 (m, 2H), 7.86-7.82 (m, 2H), 7.62 (tt, J=7.4, 1.3 Hz, 1H), 7.60-7.57 (m, 1H), 7.48 (t, J=7.8 Hz, 3H), 7.45 (t, J=7.9 Hz, 2H), 7.41-7.36 (m, 4H), 7.33 (t, J=7.8 Hz, 2H), 7.23 (dq, J=8.3, 3.3 Hz, 4H), 7.19-7.15 (m, 2H), 7.15-7.11 (m, 1H), 6.79-6.73 (m, 4H), 5.84 (d, J=3.3 Hz, 1H), 5.12 (td, J=6.8, 4.8 Hz, 1H), 5.06 (dt, J=9.6, 6.0 Hz, 1H), 4.60 (dd, J=5.5, 3.4 Hz, 1H), 4.03 (td, J=6.0, 2.6 Hz, 1H), 3.72 (s, 6H), 3.60 (dq, J=9.3, 5.9 Hz, 1H), 3.51-3.47 (m, 2H), 3.44 (s, 3H), 3.39-3.34 (m, 1H), 3.32 (dd, J=10.8, 2.8 Hz, 1H), 3.19-3.14 (m, 1H), 2.78 (ddq, J=14.2, 9.7, 4.4 Hz, 1H), 1.78 (dtt, J=12.2, 8.0, 3.8 Hz, JH), 1.64 (tdd, J=15.6, 10.8, 7.5 Hz, 1H), 1.54 (qd, J=8.6, 7.7, 3.1 Hz, 1H), 1.12 (dq, J=11.9, 9.3 Hz, 1H); ³¹P NMR (243 MHz, Acetonitrile-d3) δ 147.42; MS (ESI), 1089.03 [M −H]⁻.

### Synthesis of 3-((2R,5S,6R)-6-((Bis(4-methoxyphenyl)(phenyl)methoxy)methyl)-5-(((1S,3S,3aS)-3-((phenylsulfonyl)methyl)tetrahydro-1H,3H-pyrrolo[1,2-c][1,3,2]oxazaphosphol-1-yl)oxy)tetrahydro-2H-pyran-2-yl)pyrimidine-2,4(1H,3H)-dione

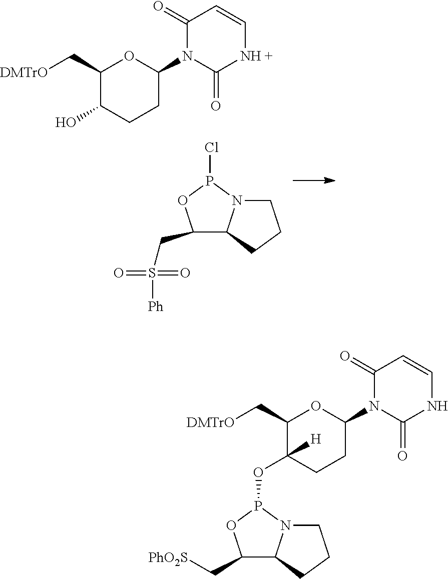

Treatment of 3-((2R,5S,6R)-6-((bis(4-methoxyphenyl)(phenyl)methoxy)methyl)-5-hydroxytetrahydro-2H-pyran-2-yl)pyrimidine-2,4(1H,3H)-dione (8.0 g, 14.69 mmol) in THF (40 mL), triethylamine (6.14 mL, 44.06 mmol) and (3S,3aS)-1-chloro-3-((phenylsulfonyl)methyl)tetrahydro-1H,3H-pyrrolo[1,2-c][1,3,2]oxazaphosphole (0.90 M in THF, 26.9 mL, 24.23 mmol) according to general procedure described above afforded 3-((2R,5S,6R)-6-((Bis(4-methoxyphenyl)(phenyl)methoxy)methyl)-5-(((1S,3S,3aS)-3-((phenylsulfonyl)methyl)tetrahydro-1H,3H-pyrrolo[1,2-c][1,3,2]oxazaphosphol-1-yl)oxy)tetrahydro-2H-pyran-2-yl)pyrimidine-2,4(1H,3H)-dione (7.86 g, 64.6%) as a white solid. ¹H NMR (600 MHz, Chloroform-d) δ 9.93 (s, 1H), 7.89-7.87 (m, 2H), 7.66-7.63 (td, J=7.3, 1.4 Hz, 1H), 7.56-7.52 (m, 4H), 7.36 (dd, J=8.88, 6.36 Hz, 4H), 7.26-7.22 (m, 2H), 7.18-7.15 (m, 1H), 6.81 (m, 4H), 6.27 (m, 1H), 6.03 (d, J=11.1 Hz, 1H), 5.36 (d, J=7.6 Hz, 1H), 4.74 (q, J=6.2 Hz, 1H), 4.15 (m, 1H), 3.74 (s, 6H), 3.61-3.56 (m, 1H), 3.5 3-3.51 (m, 1H), 3.37-3.25 (m, 4H), 3.13 (dd, J=10.5, 4.8 Hz, 1H), 2.98-2.89 (m, 2H), 2.20 (dt, J=9.3, 5.3 Hz, 1H), 1.82-1.78 (tt, J=8.3, 4.5 Hz, 1H), 1.76-1.68 (m, 3H), 1.62 (td, J=7.1, 2.9 Hz, 1H), 1.08-1.01 (m, 1H); ³¹P NMR (243 MHz, CDCl₃) δ 150.41; MS (ESI) 866.2 [M+K]⁺.

### Synthesis of 3-(bis(4-methoxyphenyl)(phenyl)methoxy)propan-1-ol (WV-LI-009)

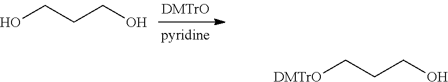

For two batches. To a solution of propane-1,3-diol (20 g, 262.83 mmol) in Py (1000 mL), DMT-C1 (62.34 g, 183.98 mmol) was added under N₂. The mixture was stirred at 25° C. for 4 hr. The two batches reaction were combined for work up. MeOH (20 mL) was added and stirred for 5 min then the mixture was concentrated to give a crude product under reduced pressure. The residue was purified by column chromatography (SiO₂, Petroleum ether:Ethyl acetate=10:1 to 1:1, 5% TEA). Compound WV-LI-009 (44 g, 23.16% yield) was obtained as a yellow oil. ¹HNMR (400 MHz, DMSO-d6) δ=7.42-7.36 (m, 2H), 7.34-7.18 (m, 7H), 6.92-6.85 (m, 4H), 4.39 (t, J=5.0 Hz, 1H), 3.73 (s, 6H), 3.55-3.48 (m, 2H), 3.05 (t, J=6.6 Hz, 2H), 1.73 (quin, J=6.5 Hz, 2H); LCMS (M+H⁺): 401.2, purity: 100%.

### Synthesis of (1S,3S,3aS)-3-(benzenesulfonylmethyl)-1-[2-[2-[2-[bis(4-methoxyphenyl)-phenyl-methoxy]ethoxy]ethoxy]ethoxy]-3a,4,5,6-tetrahydro-3H-pyrrolo[1,2-c][1,3,2]oxazaphosphole

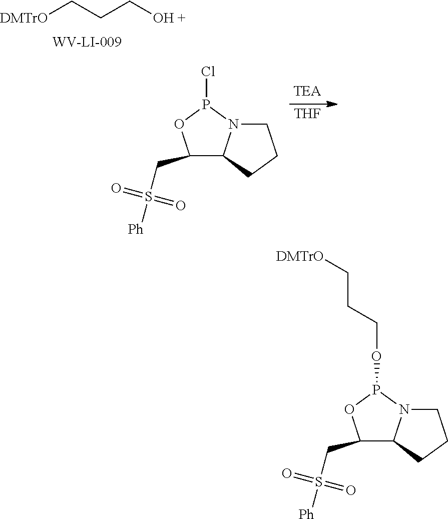

To a solution of dry 3-[bis(4-methoxyphenyl)-phenyl-methoxy]propan-1-ol (5.0 g, 13.21 mmol) in THF (37.5 mL) was added triethylamine (4.05 mL, 29.06 mmol). The reaction flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.9Min THF, 23.47 mL, 21.14 mmol) was added dropwise. The ice/water bath was removed. The resulting yellow solution was stirred at room temperature for 3 hours. TLC and LCMS showed the reaction was complete. The mixture was filtered through celite and the resulting filtrate was concentrated to give a pale yellow crude foam. The crude product was purified by normal phase column chromatography with a 20-50% EtOAc in hexanes gradient (mobile phases contained 2.5% triethylamine) to give the desired compound as a white foam. (5.58 g, 63.8% yield). ³¹P NMR (243 MHz, Chloroform-d) δ 153.97; MS (ESI), 663.16 [M+H]⁺.

### Synthesis of 4-(bis(4-methoxyphenyl)(phenyl)methoxy)butan-1-ol (WV-LI-010)

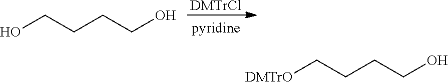

To a solution of butane-1,4-diol (40 g, 443.85 mmol) in Pyridine (1500 mL) was added DMTCl (105.27 g, 310.69 mmol). The mixture was stirred at 25° C. for 4 hr. TLC indicated compound 1 was consumed completely and two new spots formed. The reaction was clean according to TLC. The reaction mixture was added Methanol (100 ml) and concentrated under reduced pressure to give a residue. The residue was purified by column chromatography (SiO₂, Petroleum ether/Ethyl acetate=1/0 to 0/1 to Dichloromethane: Methanol=1/0 to 0/1). Compound WV-LI-010 (36 g, 60.00% yield) was obtained as a yellow oily liquid. ¹HNMR (400 MHz, DMSO-d₆) δ=7.39-7.34 (m, 2H), 7.30 (t, J=7.7 Hz, 2H), 7.26-7.20 (m, 5H), 6.95-6.81 (m, 4H), 3.73 (s, 6H), 3.38-3.33 (m, 2H), 2.94 (t, J=6.3 Hz, 2H), 1.62-1.40 (m, 4H).

### Synthesis of (1S,3S,3aS)-3-(benzenesulfonylmethyl)-1-[4-[bis(4-methoxyphenyl)-phenyl-methoxy]butoxy]-3a,4,5,6-tetrahydro-3H-pyrrolo[1,2-c][1,3,2]oxazaphosphole

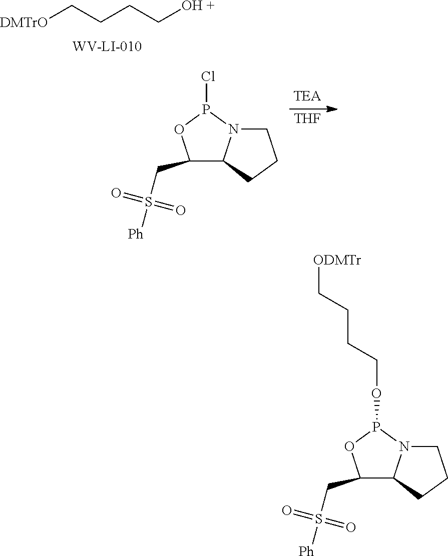

To a solution of dry 4-[bis(4-methoxyphenyl)-phenyl-methoxy]butan-1-ol (2 g, 5.10 mmol) in THF (15 mL) was added triethylamine (1.56 mL, 11.21 mmol). The reaction flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.9M in THF, 9.06 mL, 8.15 mmol) was added dropwise. The ice/water bath was removed. The resulting yellow slurry was stirred at room temperature for 3 hours. TLC and LCMS showed the reaction was complete. The reaction was quenched by water (46 L). Anhydrous MgSO₄ (620 mg) was added. The mixture was filtered through celite and the resulting clear filtrate was concentrated to give a pale yellow crude foam. The crude product was purified by normal phase column chromatography applying 10-50% EtOAc in hexanes (each mobile phase contained 2.5% triethylamine) as the gradient to give the desired product as an off-white foam (2.83 g, 82.1% yield). ³¹P NMR (243 MHz, Chloroform-d) δ 154.31; MS (ESI), 676.58 [M+H]⁺.

### Synthesis of 6-(bis(4-methoxyphenyl)(phenyl)methoxy)hexan-1-ol (WV-LI-011)

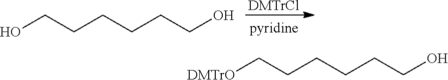

To a solution of hexane-1,6-diolin pyridine (1500 mL) was added DMTrCl (80.28 g, 236.94 mmol). The mixture was stirred at 20° C. for 4 hr. The reaction mixture was concentrated under reduced pressure to remove pyridine. The residue was purified by column chromatography (SiO₂, Petroleum ether: Ethyl acetate=1: 0 to 0: 1). Compound WV-LI-011 (46.14 g, 32.69% yield) was obtained as a yellow oil. ¹HNMR (400 MHz, CHLOROFORM-d) 6=7.40-7.35 (m, 2H), 7.30 (t, J=7.6 Hz, 2H), 7.26-7.18 (m, 5H), 6.88 (d, J=8.9 Hz, 4H), 4.31 (t, J=5.1 Hz, 1H), 3.76-3.71 (m, 6H), 3.35 (br d, J=5.4 Hz, 2H), 2.94 (t, J=6.4 Hz, 2H), 1.53 (quin, J=6.8 Hz, 2H), 1.38 (quin, J=6.9 Hz, 2H), 1.33-1.27 (m, 2H), 1.27-1.20 (m, 2H); LCMS (M−H⁺): 419.4.

### Synthesis of (1S,3S,3aS)-3-(benzenesulfonylmethyl)-1-[6-[bis(4-methoxyphenyl)-phenyl-methoxy]hexoxy]-3a,4,5,6-tetrahydro-3H-pyrrolo[1,2-c][1,3,2]oxazaphosphole

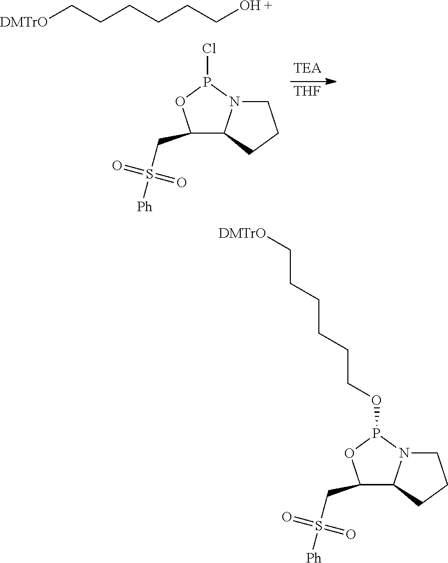

To a solution of dry 6-[bis(4-methoxyphenyl)-phenyl-methoxy]hexan-1-ol (5 g, 11.89 mmol) in THF (37.5 mL) was added triethylamine (3.65 mL, 26.16 mmol). The reaction flask was placed in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.9M in THF, 21.14 mL, 19.02 mmol) was added dropwise. The ice/water bath was removed. The resulting slurry was stirred at room temperature for 3 hours. TLC and LCMS showed that the reaction was complete. The reaction was quenched by water (107 μL). Anhydrous MgSO₄ (1.43 g) was added. The mixture was filtered through celite and the resulting clear filtrate concentrated to give a pale yellow crude foam. The crude product was purified by normal phase column chromatography applying a 20-50% EtOAc in hexanes gradient (both mobile phases contained 2.5% triethyamine) to give the desired product as an off-white solid (4.81 g, 57.5% yield). ³“P NMR (243 MHz, Chloroform-d) δ 154.49; MS (ESI) 702.40 [M−H]⁻.

### Synthesis of 2-(2-(2-(bis(4-methoxyphenyl)(phenyl)methoxy)ethoxy)ethoxy)ethan-1-ol (WV-DL-012)

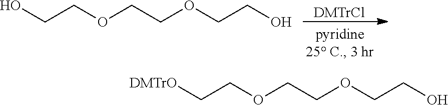

For four batches. To a solution of 2,2′-(ethane-1,2-diylbis(oxy))bis(ethan-1-ol) (10 g, 66.59 mmol) in Py (500 mL), DMTrCl (15.79 g, 46.61 mmol) was added under N₂. The mixture was stirred at 25° C. for 4 hr. The four batches reaction were combined for work up. MeOH (20 mL) was added and stirred for 5 min then the mixture was concentrated to give a crude product under reduced pressure. The residue was purified by column chromatography (SiO₂, Petroleum ether:Ethyl acetate=10:1 to 1:1, 5% TEA). Compound WV-LI-012 (22 g, 23.16% yield) was obtained as a yellow oil. ¹HNMR (400 MHz, DMSO-d6) δ=7.41 (d, J=7.4 Hz, 2H), 7.35-7.19 (m, 7H), 6.89 (d, J=8.9 Hz, 4H), 4.60 (t, J=5.3 Hz, 1H), 3.74 (s, 6H), 3.62-3.43 (m, 10H), 3.06 (t, J=4.9 Hz, 2H).

### Synthesis of (1S,3S,3aS)-3-(benzenesulfonylmethyl)-1-[2-[2-[2-[bis(4-methoxyphenyl)-phenyl-methoxy]ethoxy]ethoxy]ethoxy]-3a,4,5,6-tetrahydro-3H-pyrrolo[1,2-c][1,3,2]oxazaphosphole

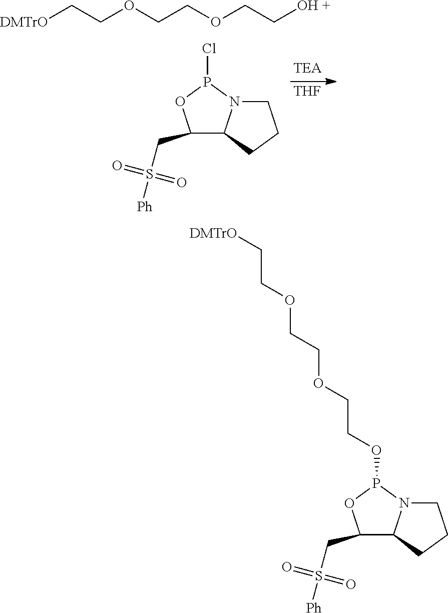

To a solution of dry 2-[2-[2-[bis(4-methoxyphenyl)-phenyl-methoxy]ethoxy]ethoxy]ethanol in THF (15 mL) was added triethylamine (1.48 mL, 10.61 mmol). The reaction flask was set in an ice/water bath. (3S,3aS)-3-(benzenesulfonylmethyl)-1-chloro-3a,4,5,6-tetrahydro-3H-pyrrolo[1,2-c][1,3,2]oxazaphosphole (0.9M in THF, 8.84 mL, 7.96 mmol) was added dropwise. The ice/water bath was removed. The resulting yellow slurry was at room temperature for 3 hours. TLC and LCMS showed the reaction was complete. The reaction mixture was filtered through celite and the resulting cloudy filtrate was concentrated to give a pale yellow crude foam. The crude product was purified by normal phase column chromatography applying a 30-60% EtOAc in hexanes gradient (both mobile phases contained 2.5% triethylamine) to afford the desired product as a yellow oil (1.07 g, 32.9% yield). ³¹P NMR (243 MHz, Chloroform-d) δ 154.29; MS (ESI) 736.35 [M+H]⁺.

### Example 3. Preparation of Oligonucleotide and Compositions

Various technologies for preparing oligonucleotides and oligonucleotide compositions (both stereorandom and chirally controlled) can be utilized in accordance with the present disclosure, including, for example, methods and reagents described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2021/237223, WO 2022/099159, and/or PCT/US2023/018742, the methods and reagents of each of which are incorporated herein by reference.

In some embodiments, preparations include one or more DPSE and/or PSM cycles. Certain cycle conditions are provided below as examples; those skilled in the art appreciate that such conditions may be optimized or modified (e.g., reagents, concentration, etc.).

Regular Amidite Synthetic Cycle

| step | operation | reagents and solvent |
|---|---|---|
| 1 | detritylation | 3% TCA/DCM |
| 2 | coupling | 0.2M monomer/20% IBN-MeCN |
| | | 0.5M CMIMT/MeCN |
| 3 | oxidation | 50 mM I₂/pyridine-H₂O (9:1, v/v) |
| 4 | cap-2 | 20% Ac₂O, 30% 2,6-lutidine/MeCN |
| | | 20% MeIm/MeCN |

DPSE Amidite Synthetic Cycle

| step | operation | reagents and solvent |
|---|---|---|
| 1 | detritylation | 3% TCA/DCM |
| 2 | coupling | 0.2M monomer/20% IBN-MeCN |
| | | 0.5M CMIMT/MeCN |
| 3 | cap-1 | 20% Ac₂O, 30% 2,6-lutidine/MeCN |
| 4 | sulfurization | 0.2M XH/pyridine |
| 5 | cap-2 | 20% Ac₂O, 30% 2,6-lutidine/MeCN |
| | | 20% MeIm/MeCN |

PSM Amidite Synthetic Cycle

| step | operation | reagents and solvent |
|---|---|---|
| 1 | detritylation | 3% TCA/DCM |
| 2 | coupling | 0.2M monomer/20% IBN-MeCN |
| | | 0.5M CMIMT/MeCN |
| 3 | cap-1 | 20% Ac₂O, 30% 2,6-lutidine/MeCN |
| 4 | imidation | 0.5M ADIH reagent/MeCN |
| 5 | cap-2 | 20% Ac₂O, 30% 2,6-lutidine/MeCN |
| | | 20% MeIm/MeCN |

In some embodiments, for introduction of additional chemical moieties, e.g., GalNAc moieties, at 5′-end, oligonucleotides are synthesized by coupling a suitable linker, e.g., a C-6 amino linker, as the last coupling cycle.

After completion of cycles, the CPG support is treated with an anhydrous amine solution, e.g., 20% diethylamine/acetonitrile to remove PSM chiral auxiliaries, cyanoethyl groups, etc.. DPSE can be removed with desilylation reagent comprising HF (e.g., DMSO:water:TEA:TEA.3HF in ratio of 7.33:1.47:0.7:0.5). The CPG can then be treated with conc. NH₃ (5.0 mL; 200 μL/umol) for a suitable period time at a suitable temperature, e.g., 24 h at 37° C. In some embodiments, the reaction mixture is cooled to room temperature and the CPG is separated by membrane filtration and washed with H₂O. In some embodiments, the crude material (filtrate) is analyzed by LTQ and RP-UPLC. For certain oligonucleotides to be conjugated with other additional chemical moieties such as GalNac, oligonucleotides comprising suitable reactive groups such as amino groups can be purified, e.g., by ion exchange chromatography on AKTA pure system using a sodium chloride gradient. Desired product can be desalted and further conjugated with GalNAc-containing acid. After conjugation reaction is found to be completed, the material can be further purified by ion exchange chromatography and desalted using tangential flow filtration (TFF) to obtain desired products.

Product compositions can be assessed and characterized by various technologies, e.g., UV, HPLC, UPLC, MS, NMR, etc.

Many oligonucleotides and compositions, e.g., various oligonucleotides and compositions thereof in Table 1, were prepared and assessed and were confirmed to provide various activities, e.g., adenosine editing.

### Example 4. Provided technologies can provide editing

Among other things, provided technologies can provide efficient editing of target adenosine. Various technologies can be utilized to assess, characterize, and confirm properties and activities of provided technologies.

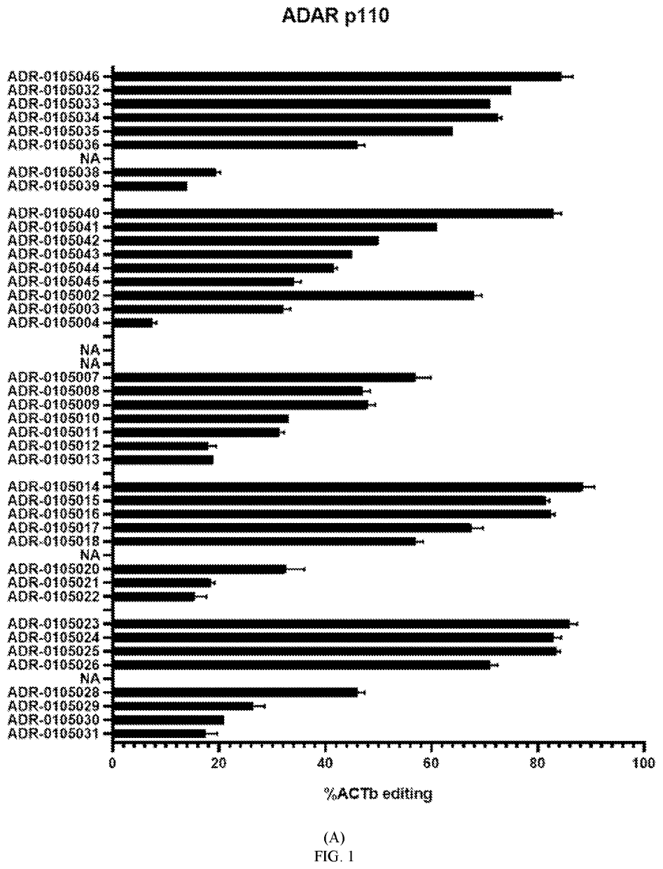

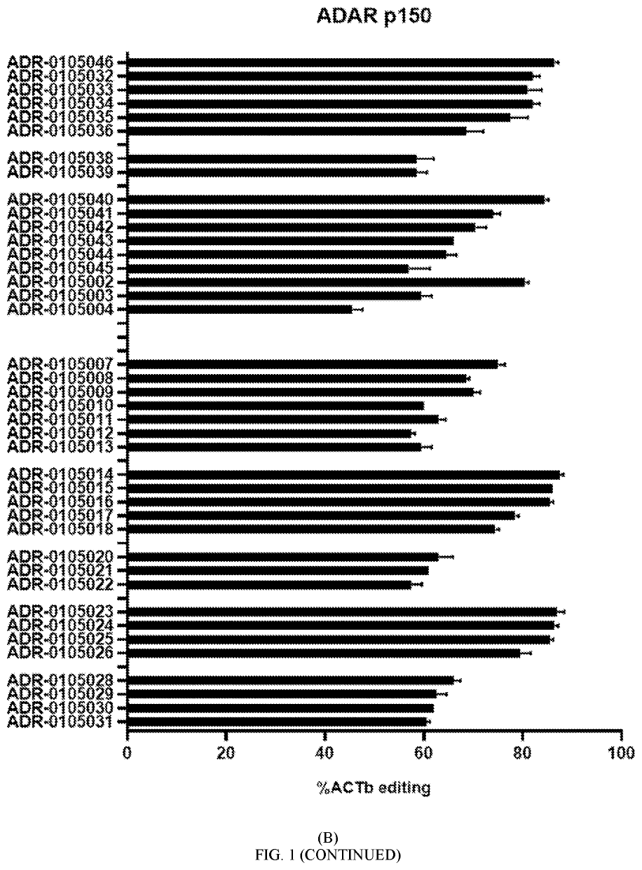

For example, in an assessment, HEK293T cells were transfected with 1 μg of either ADAR p110 or ADAR p150 expression plasmids. At 8 hours post-plasmid transfection, various oligonucleotide compositions (20 nM) were transfected into the HEK293T cells expressing ADAR p110 or ADAR p150. All transfections were conducted in biological replicate (n=2). After 48 hours, cells were harvested, RNA was extracted, cDNA was generated, and polymerase chain reaction (PCR) was performed as known in the art to isolate copies of ACTB transcript. Editing of ACTB transcript was assessed using Sanger sequencing and analysis of resulting sequencing data. As demonstrated in FIG. 1, provided oligonucleotide compositions can provide robust target adenosine editing.

### Example 5. Provided Technologies can Provide Editing

Oligonucleotides comprising various types of sugars, nucleobase, internucleotidic linkages, and stereochemistry and patterns thereof were designed and assessed, including those comprising PO internucleotidic linkages (e.g., natural phosphate linkages), PS (e.g., phosphorothioate) internucleotidic linkages, PN (e.g., phosphoryl guanidine linkages such as n001) internucleotidic linkages, various types of nucleobases (e.g., at No position such as 3nU, etc.), various types of sugars (e.g., 2′-F modified sugars, 2′-OMe modified sugars, natural DNA sugars, LNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, 2′-MOE, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), etc. as described herein. In some embodiments, oligonucleotides are designed with a first domain comprising an about 15 nucleotide sequence fully complementary to a sequence region including a target NRF2 adenosine and a second domain comprising an about 15 nucleotide sequence fully complementary to a region 3′ distant to the target adenosine, wherein complementary portions of the two domains in a NRF2 transcript are separated by gaps of various lengths.

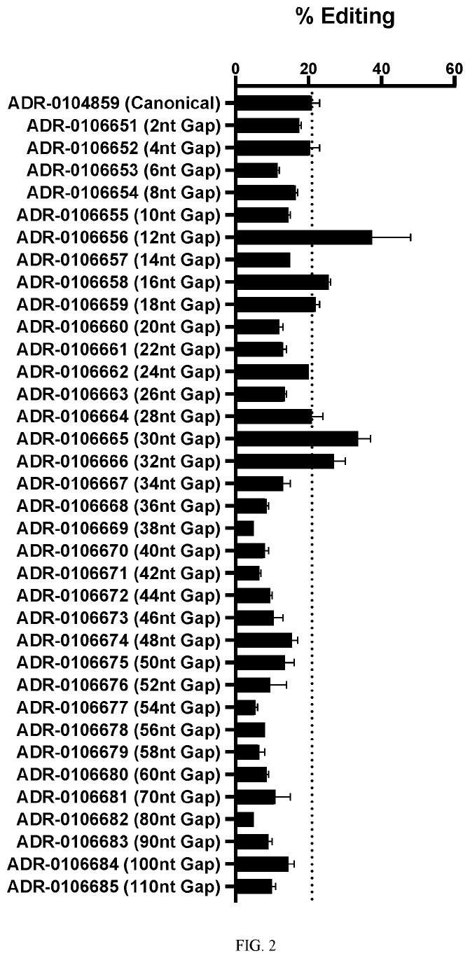

Certain data are presented in FIG. 2, confirming that provided technologies can provide editing, and in various embodiments, oligonucleotides comprising first and second domains whose corresponding first and second portions are separated by gaps of various lengths can provide effective editing. In some embodiments, oligonucleotides comprising various gap lengths between a first domain and second domain of the oligonucleotide can provide comparable or increased editing relative to a reference (e.g., oligonucleotides without a gap). Human primary astrocytes expressing endogenous ADAR were dosed with compositions comprising indicated oligonucleotides targeting NRF2 via transfection at a dosage of 25 nM. Cells were harvested 48 h later, and RNA was collected and reverse transcribed into cDNA. Editing was assessed by Sanger sequencing.

In some embodiments, it was further confirmed that oligonucleotides of various structural patterns, including those comprising first and second domains whose corresponding first and second portions are separated by gaps of various lengths, can provide effective editing. In some embodiments, oligonucleotides comprising a gap of about 2 nucleotides in length to about 35 nucleotides in length can provide effective editing. In some embodiments, oligonucleotides comprising a gap of up to about 110 nucleotides in length can provide effective editing. In some embodiments, oligonucleotides comprising a gap of at least about 2 nucleotides in length can provide effective editing.

### Example 6. Provided Technologies can Provide Editing

Oligonucleotides comprising various types of sugars, nucleobase, internucleotidic linkages, and stereochemistry and patterns thereof were designed and assessed, including those comprising PO internucleotidic linkages (e.g., natural phosphate linkages), PS (e.g., phosphorothioate) internucleotidic linkages, PN (e.g., phosphoryl guanidine linkages such as n001) internucleotidic linkages, various types of nucleobases (e.g., at No position such as 3nU, etc.), various types of sugars (e.g., 2′-F modified sugars, 2′-OMe modified sugars, natural DNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, 2′-MOE, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), etc. as described herein. In some embodiments, oligonucleotides are designed to comprise a first domain and second domain, wherein the first domain is complementary to a sequence region including a target adenosine and the second domain is complementary to a sequence region including a second target adenosine. As described herein, in some embodiments, an oligonucleotide may comprise more than one editing region. In some embodiments, a domain may comprise more than one editing region. In some embodiments, a first domain comprises an editing region as described herein. In some embodiments, a second domain comprises an editing region as described herein. In some embodiments, a first domain and a second domain each independently comprises an editing region. In some embodiments, a first domain editing region targets a first target adenosine, and a second domain editing region targets a second target adenosine. In some embodiments, a first domain comprises a 5′-N₁N₀N₋₁-3′ as described herein, and a second domain comprises a 5′-N₁N₀N₋₁-3′ as described herein. In some embodiments, a first target adenosine and a second target adenosine is separated by a gap, e.g., of about 1-10, 5-15, 10-20, 15-30, 25-50, 40-100, 50-200, 150-500, 250-1000, or more nucleobases in a target transcript. As confirmed herein, provided technologies can be utilized to selectively editing more than one target adenosine.

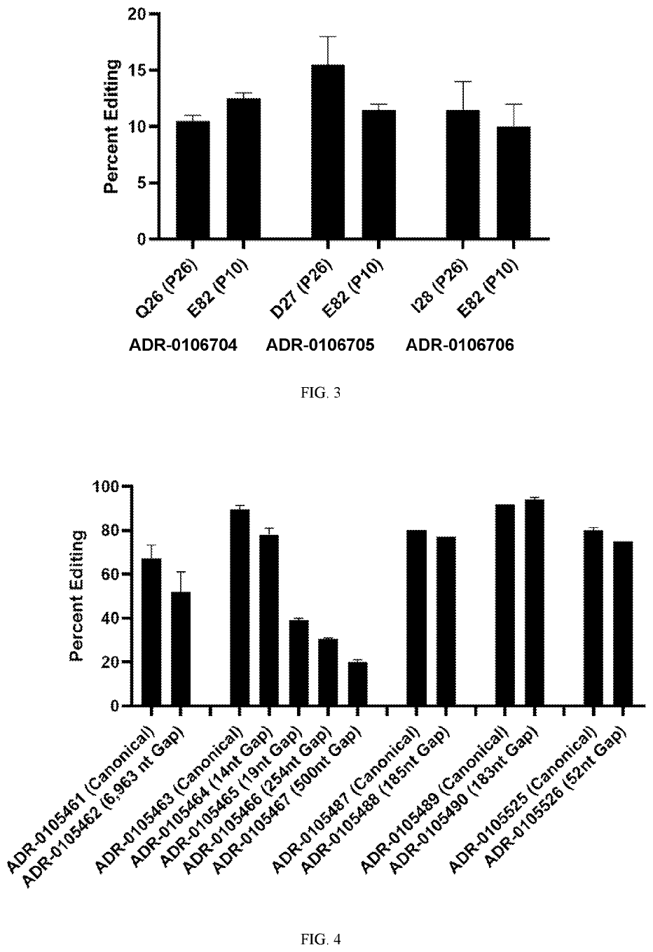

Certain data are presented in FIG. 3, confirming that provided technologies can provide editing, and in various embodiments, oligonucleotides comprising a first domain and second domain, wherein the first domain is complementary to a sequence region including a target adenosine and the second domain is complementary to a sequence region including a second target adenosine can provide effective editing. Human primary astrocytes expressing endogenous ADAR were dosed with compositions comprising indicated oligonucleotides targeting NRF2 via transfection at a dosage of 25 nM. Cells were harvested 48 h later, and RNA was collected and reverse transcribed into cDNA. Editing was assessed by Sanger sequencing.

In some embodiments, it was confirmed that oligonucleotides of various structural patterns, including those comprising a first domain and second domain, wherein the first domain is complementary to a sequence region including a target NRF2 adenosine and the second domain is complementary to a sequence region including a second target NRF2 adenosine can provide effective editing, can provide effective editing. In some embodiments, provided oligonucleotides can provide effective editing at both target adenosine sites. In some embodiments, such oligonucleotides can provide comparable editing at both target adenosine sites. In some embodiments, oligonucleotides comprising a gap of about 150 or more nucleotides in length can provide editing. In some embodiments, oligonucleotides comprising a gap of up to about 150 nucleotides in length can provide editing. In some embodiments, oligonucleotides comprising a gap of at least about 150 nucleotides in length can provide editing. In some embodiments, oligonucleotides comprising a gap of about 150 nucleotides in length can be utilized to edit at two target adenosine sites. In some embodiments, oligonucleotides can provide editing at a first target adenosine and a second target adenosine. In some embodiments, a first portion comprising a first target adenosine and a second portion comprising a second target adenosine are separated by a gap of various lengths, e.g., at least about 10, 20, 30, 40, 50, 60, 70, 80, 90, 100, 110, 120, 130, 140, 150, 160, 170, 180, 190, or 200 nucleotides in length.

### Example 7. Provided Technologies can Provide Editing

Oligonucleotides comprising various types of sugars, nucleobase, internucleotidic linkages, and stereochemistry and patterns thereof were designed and assessed, including those comprising PO e.g., (natural phosphate linkages) internucleotidic linkages, PS (e.g., phosphorothioate) internucleotidic linkages, PN (e.g., phosphoryl guanidine linkages such as n001) internucleotidic linkages, various types of nucleobases (e.g., at No position such as 3nU, etc.), various types of sugars (e.g., 2′-F modified sugars, 2′-OMe modified sugars, natural DNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, 2′-MOE, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), etc. as described herein. In some embodiments, oligonucleotides are designed with a first domain comprising about 15 nt sequence fully complementary to a sequence region including a target Malat1 adenosine and a second domain comprising about 15 nt sequence fully complementary to a sequence region 3′ distant to the target adenosine, wherein the two domains comprised intervening gaps of various lengths.

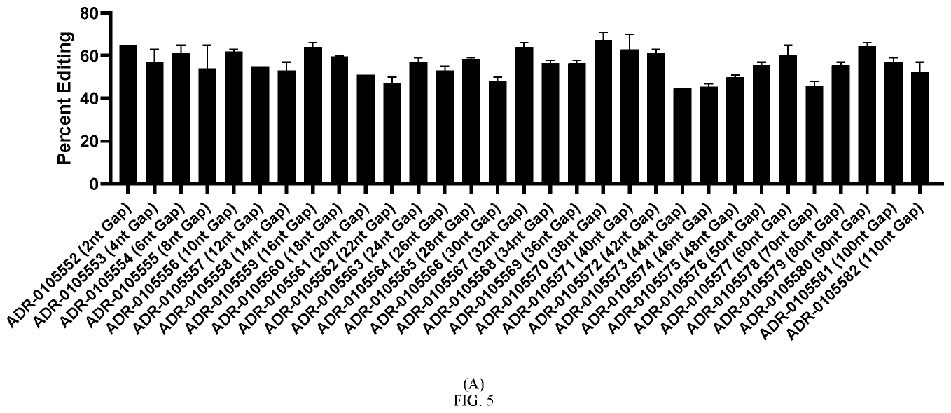

Certain data are presented in FIG. 4, confirming that provided technologies can provide editing, and in various embodiments, oligonucleotides comprising first and second domains whose corresponding first and second portions are separated by gaps of various lengths can provide effective editing. In some embodiments, oligonucleotides comprising various gap lengths between a first domain and second domain of the oligonucleotide can provide comparable or increased editing relative to a reference (e.g., oligonucleotides without a gap). In some embodiments, such oligonucleotides may provide editing at one or more target adenosines within a transcript. HEK293T cells were transfected with a plasmid encoding ADAR1 p110 for exogenous ADAR expression. After 24 hours, cells were transfected with compositions comprising indicated oligonucleotides targeting Malat1 via transfection at a dosage of 25 nM. Cells were harvested 48 h later, and RNA was collected and reverse transcribed into cDNA. Editing was assessed by Sanger sequencing.

In some embodiments, it was confirmed that oligonucleotides of various structural patterns, including those comprising first and second domains whose corresponding first and second portions are separated by gaps of various lengths, can provide effective editing. In some embodiments, such oligonucleotides may comprise a gap of about or greater than about 100 nucleotides in length. In some embodiments, such oligonucleotides may comprise a gap of about or greater than about 1000 nucleotides in length. In some embodiments, such oligonucleotides may comprise a gap of about or greater than about 6000 nucleotides in length. In some embodiments, such oligonucleotide designs may be guided by predicted secondary and tertiary structures of a target transcript.

### Example 8. Provided Technologies can Provide Editing

Oligonucleotides comprising various types of sugars, nucleobase, internucleotidic linkages, and stereochemistry and patterns thereof were designed and assessed, including those comprising PO internucleotidic linkages (e.g., natural phosphate linkages), PS (e.g., phosphorothioate) internucleotidic linkages, PN (e.g., phosphoryl guanidine linkages such as n001) internucleotidic linkages, various types of nucleobases (e.g., at No position such as 3nU, etc.), various types of sugars (e.g., 2′-F modified sugars, 2′-OMe modified sugars, natural DNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, 2′-MOE, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), etc. as described herein. In some embodiments, oligonucleotides are designed with a first domain comprising an about 15 (e.g., 15, 16, etc.) nucleotide sequence fully complementary to a sequence region including a target MECP2 adenosine and a second domain comprising an about 15 nucleotide (e.g., 15, 16, etc.) nucleotide sequence fully complementary to a sequence region 3′ distant to the target adenosine, wherein intervening gaps of various lengths were between the sequence regions.

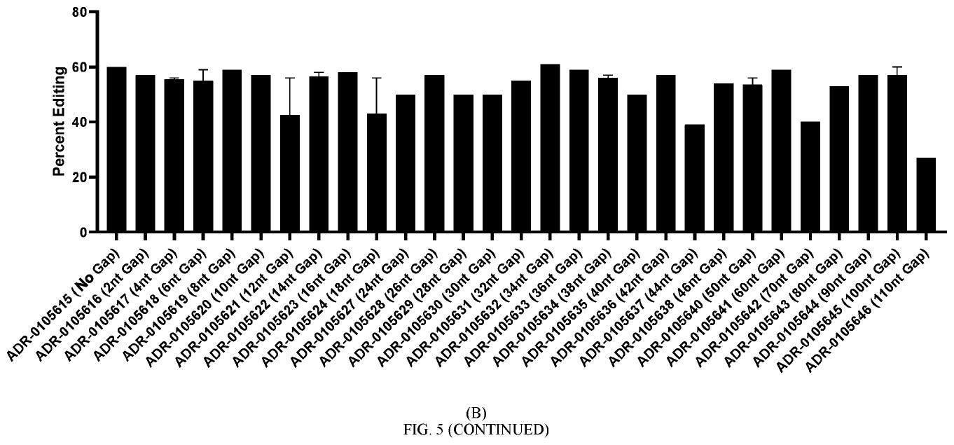

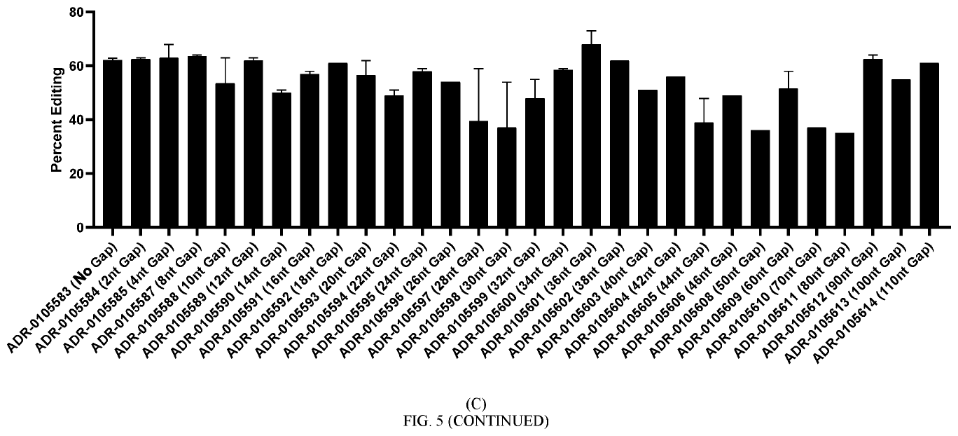

Certain data are presented in FIG. 5, confirming that provided technologies can provide editing, and in various embodiments, oligonucleotides comprising first and second domains whose corresponding first and second portions are separated by gaps of various lengths can provide effective editing. In some embodiments, oligonucleotides comprising various gap lengths between a first domain and second domain of the oligonucleotide can provide comparable or increased editing relative to a reference (e.g., oligonucleotides without a gap). In some embodiments, such oligonucleotides may provide editing at one or more target adenosines within a transcript. HEK293T cells were transfected with a plasmid encoding ADAR1 p110 for exogenous ADAR expression and a plasmid encoding MECP2 for exogenous expression of MECP2. After 24 hours, cells were transfected with compositions comprising indicated oligonucleotides targeting MECP2 via transfection at a dosage of 25 nM. Cells were harvested 48 h later, and RNA was collected and reverse transcribed into cDNA. Editing was assessed by Sanger sequencing.

In some embodiments, it was confirmed that oligonucleotides of various structural patterns, including those comprising first and second domains whose corresponding first and second portions are separated by gaps of various lengths, can provide effective editing. In some embodiments, it was further confirmed that oligonucleotides of various structural patterns, including those comprising first and second domains who comprise various numbers of nucleotides and whose corresponding first and second portions are separated by gaps of various lengths, can provide effective editing. In some embodiments, such oligonucleotides may comprise a first domain of about 5 to 25 nucleotides, about 10 to 20 nucleotides, about 15 to 16 nucleotides, about 15 nucleotides, or about 16 nucleotides. In some embodiments, such oligonucleotides may comprise a second domain of about 5 to 25 nucleotides, about 10 to 20 nucleotides, about 15 to 16 nucleotides, about 15 nucleotides, or about 16 nucleotides. In some embodiments, an oligonucleotide may In some embodiments, such oligonucleotides may comprise a gap of about or at least about 2, 4, 6, 8, 10, 12, 14, 16, 18, 20, 22, 24, 26, 28, 30, 32, 34, 36, 38, 40, 42, 44, 46, 48, 50, 60, 70, 80, 90, 100, or 110 nucleotides in length. In some embodiments, such oligonucleotides may comprise a gap of about 2 nucleotides to about 110 nucleotides in length. In some embodiments, such oligonucleotides may comprise a gap of greater than about 100 nucleotides in length. In some embodiments, such oligonucleotide designs may be guided by predicted secondary and tertiary structures of a target transcript.

### Example 9. Provided Technologies can Provide Editing

Oligonucleotides comprising various types of sugars, nucleobase, internucleotidic linkages, and stereochemistry and patterns thereof were designed and assessed, including those comprising PO internucleotidic linkages (e.g., natural phosphate linkages), PS (e.g., phosphorothioate) internucleotidic linkages, PN (e.g., phosphoryl guanidine linkages such as n001) internucleotidic linkages, various types of nucleobases (e.g., at N₀ position such as 3nU, etc.), various types of sugars (e.g., 2′-F modified sugars, 2′-OMe modified sugars, natural DNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), various linkers (e.g., oligonucleotides comprising various linkers, e.g., [SpC3], [oC4o], [oC6o], [Sp9], or [Sp12], between first and second domains) etc. as described herein. In some embodiments, oligonucleotides are designed with a first domain comprising an about 15 (e.g., 15, 16, etc.) nucleotide sequence fully complementary to a sequence region including a target MECP2 adenosine and a second domain comprising an about 15 (e.g., 15, 16, etc.) nucleotide sequence fully complementary to a sequence region 3′ distant to the target adenosine, wherein intervening gaps of various lengths were between the sequence regions.

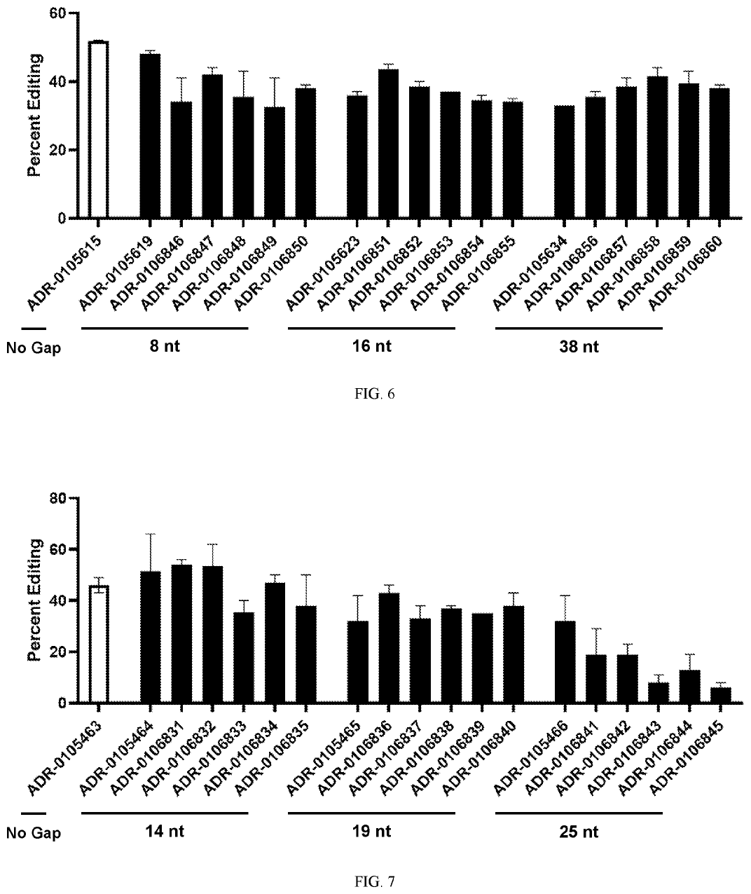

Certain data are presented in FIG. 6, confirming that provided technologies can provide editing, and in various embodiments, oligonucleotides comprising first and second domains whose corresponding first and second portions are separated by gaps of various lengths can provide effective editing. In some embodiments, oligonucleotides comprising various gap lengths between a first domain and second domain of the oligonucleotide can provide comparable or increased editing relative to a reference (e.g., oligonucleotides without a gap). In some embodiments, oligonucleotides comprising various linkers between a first domain and second domain of the oligonucleotide can provide comparable or increased editing relative to a reference (e.g., oligonucleotides without a linker). In some embodiments, such oligonucleotides may provide editing at one or more target adenosines within a transcript. HEK293T cells were transfected with a plasmid encoding ADAR1 p110 for exogenous ADAR expression and a plasmid encoding MECP2 for exogenous expression of MECP2. After 24 hours, cells were transfected with compositions comprising indicated oligonucleotides targeting MECP2 via transfection at a dosage of 25 nM. Cells were harvested 48 h later, and RNA was collected and reverse transcribed into cDNA. Editing was assessed by Sanger sequencing.

In some embodiments, it was confirmed that oligonucleotides of various structural patterns, including those comprising first and second domains whose corresponding first and second portions are separated by gaps of various lengths, can provide effective editing. In some embodiments, it was further confirmed that oligonucleotides of various structural patterns, including those comprising first and second domains, whose corresponding first and second portions are separated by gaps of various lengths, and a linker between first and second domains, can provide effective editing. In some embodiments, such oligonucleotides comprise a linker, e.g., [SpC3], [oC4o], [oC6o], [Sp9], or [Sp12]. In some embodiments, such oligonucleotides may comprise a gap of about or at least about 8, 16, or 38 nucleotides in length. In some embodiments, such oligonucleotides may comprise a gap of about 8 nucleotides to about 38 nucleotides in length. In some embodiments, such oligonucleotides may comprise a gap of greater than about 35 nucleotides in length. In some embodiments, such oligonucleotide designs may be guided by predicted secondary and tertiary structures of a target transcript. In some embodiments, an oligonucleotide comprising a linker, e.g., [SpC3], [oC4o], [oC6o], [Sp9], or [Sp12], can provide editing.

### Example 10. Provided Technologies can Provide Editing

Oligonucleotides comprising various types of sugars, nucleobase, internucleotidic linkages, and stereochemistry and patterns thereof were designed and assessed, including those comprising PO internucleotidic linkages (e.g., natural phosphate linkages), PS (e.g., phosphorothioate) internucleotidic linkages, PN (e.g., phosphoryl guanidine linkages such as n001) internucleotidic linkages, various types of nucleobases (e.g., at No position such as 3nU, etc.), various types of sugars (e.g., 2′-F modified sugars, 2′-OMe modified sugars, natural DNA sugars, etc.), controlled stereochemistry, patterns thereof (e.g., for sugars, 2′-F blocks and 2′-OR (e.g., wherein R is optionally substituted C₁₋₆ aliphatic (e.g., 2′-OMe, etc.)) blocks, or patterns thereof, at 5′-sides (e.g., first domains and/or first subdomains of second domains) and/or 3′-sides of editing regions or second subdomains of second domains or 5′-N₁N₀N₋₁-3′), various gap lengths (e.g., oligonucleotides comprising first and second domains whose corresponding first and second portions are separated by gaps of various lengths), various linkers (e.g., oligonucleotides comprising various linkers, e.g., [SpC3], [oC4o], [oC6oj, [Sp9], or [Sp12], between first and second domains) etc. as described herein. Oligonucleotides designed with a first domain comprising a 16 nucleotide sequence fully complementary to a sequence region including a target Malat1 adenosine and a second domain comprising a 15 nucleotide sequence fully complementary to a sequence region 3′ distant to the target adenosine, wherein intervening gaps of various lengths were between the sequence regions.

Certain data are presented in FIG. 7, confirming that provided technologies can provide editing, and in various embodiments, oligonucleotides comprising first and second domains whose corresponding first and second portions are separated by gaps of various lengths can provide effective editing. In some embodiments, oligonucleotides comprising various gap lengths between a first domain and second domain of the oligonucleotide can provide comparable or increased editing relative to a reference (e.g., oligonucleotides without a gap). In some embodiments, oligonucleotides comprising various linkers between a first domain and second domain of the oligonucleotide can provide comparable or increased editing relative to a reference (e.g., oligonucleotides without a linker). In some embodiments, such oligonucleotides may provide editing at one or more target adenosines within a transcript. HEK293T cells were transfected with a plasmid encoding ADAR1 p110 for exogenous ADAR expression. After 24 hours, cells were transfected with compositions comprising indicated oligonucleotides targeting Malat1 via transfection at a dosage of 25 nM. Cells were harvested 48 h later, and RNA was collected and reverse transcribed into cDNA. Editing was assessed by Sanger sequencing.

In some embodiments, it was confirmed that oligonucleotides of various structural patterns, including those comprising first and second domains whose corresponding first and second portions are separated by gaps of various lengths, can provide effective editing. In some embodiments, it was further confirmed that oligonucleotides of various structural patterns, including those comprising first and second domains, whose corresponding first and second portions are separated by gaps of various lengths, and a linker between first and second domains, can provide effective editing. In some embodiments, such oligonucleotides comprise a linker, e.g., [SpC3], [oC4o], [oC6o], [Sp9], or [Sp12]. In some embodiments, such oligonucleotides may comprise a gap of about or at least about 14, 19, or 25 nucleotides in length. In some embodiments, such oligonucleotides may comprise a gap of about 14 nucleotides to about 25 nucleotides in length. In some embodiments, such oligonucleotides may comprise a gap of greater than about 20 nucleotides in length. In some embodiments, such oligonucleotide designs may be guided by predicted secondary and tertiary structures of a target transcript. In some embodiments, an oligonucleotide comprising a linker, e.g., [SpC3], [oC4o], [oC6o], [Sp9], or [Sp12], can provide editing.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An oligonucleotide, comprising:
a first domain, wherein the length of the first domain is about 10 nucleobases or more;
a second domain, wherein the length of the second domain is about 10 nucleobases or more; and
a 2′-F modified sugar;
wherein:
the base sequence of the first domain is complementary to a first portion of the base sequence of a target nucleic acid;
the base sequence of the second domain is complementary to a second portion of the base sequence of the target RNA nucleic acid;
the first portion and the second portion of the base sequence of the target RNA nucleic acid are separated by a gap of about or at least about 50, 100, 200, 500 or 1000 nucleobases in the target nucleic acid; and
the length of the oligonucleotide is about 25-50 nucleobases.

2. An oligonucleotide comprising:
a first domain; and
a second domain,
wherein:
the base sequence of the first domain is complementary to a first portion of the base sequence of a target nucleic acid;
the base sequence of the second domain is complementary to a second portion of the base sequence of a target nucleic acid; and
the first portion and the second portion of the base sequence of the target nucleic acid are separated by a gap.

3. An oligonucleotide comprising:
a first domain; and
a second domain,
wherein:
the first domain is characterized in that it can form a duplex with a first portion of a target nucleic acid;
the second domain is characterized in that it can form a duplex with a second portion of a target nucleic acid; and
the first portion and the second portion of the base sequence of the target nucleic acid are separated by a gap.

4. The oligonucleotide of any one of claims 1-3, wherein the oligonucleotide comprises 5′-N₁N₀N₋₁-3′, wherein each of N₋₁, N₀, and N₁ is independently a nucleoside; and wherein the nucleobase of N₀ is BA, wherein BA is

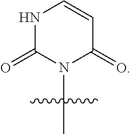 [SMILES: *n1c(=O)cc[nH]c1=O]

5. The oligonucleotide of any one of claims 1-3, wherein the oligonucleotide comprises 5′-N₁N₀N₋₁-3′, wherein each of N₋₁, N₀, and N₁ is independently a nucleoside; and wherein the nucleobase of N₀ is BA, wherein BA comprises Ring BA or a tautomer thereof, wherein Ring BA has the structure of formula BA-III-e: 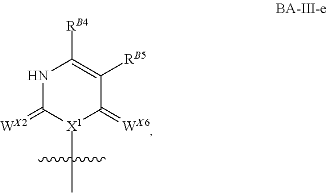
wherein:
X¹ is —N(−)- or —C(−)=;
each of W^{X2} and W^{X6} is independently O, S or Se;
R^{B4} is halogen, —CN, —NO₂, or -L^{B4}-R^{B41}, wherein R^{B41} is R′;
R^{B5} is halogen, —CN, —NO₂, or -L^{B5}-R^{B51}, wherein R^{B51} is -R′, —N(R′)₂, —OR′, or -SR′,
each of L^{B4} and L^{B5} is independently L^{B};
each L^{B} is independently a covalent bond, or an optionally substituted bivalent C₁₋₁₀ saturated or partially unsaturated chain having 0-6 heteroatoms, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted, 3-20 membered, monocyclic, bicyclic or polycyclic ring having 0-10 heteroatoms;
each R′ is independently —R, —C(O)R, —C(O)OR, —C(O)N(R)₂, or —SO₂R; and
each R is independently —H, or an optionally substituted group selected from C₁₋₂₀ aliphatic, C₁₋₂₀ heteroaliphatic having 1-10 heteroatoms, C₆₋₂₀ aryl, C6-20 arylaliphatic, C6-2o arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

6. The oligonucleotide of claim 4 or 5, wherein the sugar of N₀ comprises a 2′-OR modification, wherein R is optionally substituted C₁₋₆ aliphatic.

7. The oligonucleotide of any one of claims 1-3, wherein the oligonucleotide comprises 5′-N₁N₀N₋₁-3′, wherein each of N₋₁, N₀, and N₁ is independently a nucleoside; and wherein the nucleobase of N₀ is BA, wherein BA is

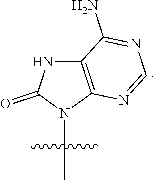 [SMILES: *n1c(=O)[nH]c2c(N)ncnc21]

8. The oligonucleotide of any one of claims 1-3, wherein the oligonucleotide comprises 5′-N₁N₀N₋₁-3′, wherein each of N₋₁, N₀, and N₁ is independently a nucleoside; and wherein the nucleobase of N₀ is BA, wherein BA comprises Ring BA or a tautomer thereof, wherein Ring BA has the structure of formula BA-VI: 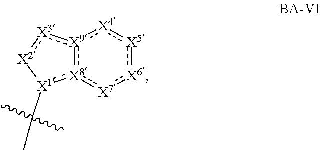
wherein:
each [symbol] is independent a single or double bond;
X¹′ is —N(−)— or —C(−)═;
X^{2′} is —C(W^{X2}′)—, —C(R^{B2}′)═, —C(OR^{B2}′)═, —N=, or optionally substituted —CH═ or —CH₂—, wherein R^{B2}′ is halogen, —CN, —NO₂, or -L^{B2}′-R′, and W^{X2}′ is O, S or Se;
X³′ is —N(R^{B3}′)-, —N═, —C(R^{B3}′)═ or optionally substituted —NH— or —CH═, wherein R^{B3}′ is halogen, —CN, —NO₂, or -L^{B3}′-R′;
X⁴′ is —C(R^{B4}′)═, —C(OR^{B4}′)═, —C(—N(R^{B4}′)₂)═, —C(R^{B4}′)₂—, —C(W^{X4}′)—, —C(═NR^{B4}′)—, —N(R^{B4}′)—, —N═, or optionally substituted —CH═, —NH— or —CH₂—, wherein each R^{B4}′ is independently halogen, —CN, —NO₂, or -L^{B4′}-R^{B41}′ or two R^{B4}′ on the same atom are taken together to form ═O, ═C(-L^{B4}′-R^{B41}′)₂, ═N-L^{B4}′-R^{B41}′, or optionally substituted ═CH₂ or ═NH, wherein each R^{B41}′ is independently —R′, and W^{X4}′ is O, S or Se;
X⁵′ is —C(R^{B5}′)₂—, —N(R^{B5}′)—, —C(R^{B5}′)═, —C(W^{X5}′)—, —N═, or optionally substituted —NH—, —CH₂—, or —CH═, wherein each R^{B5}′ is independently halogen, —CN, —NO₂, or -L^{B5}′-R^{B51}′, wherein R^{B51}′ is —R′, —N(R′)₂, —OR′, or —SR′, and W^{X5}′ is O, S, or Se;
X^{6′} is —C(R^{B6}′)═, —C(OR^{B6}′)═, —C(R^{B6}′)₂—, —C(W^{X6}′)—, —C(—N(R^{B6}′)₂)═, —N═ or optionally substituted —NH—, —CH₂— or —CH═, wherein each R^{B6}′ is independently halogen, —CN, —NO₂, or -L^{B6}′-R^{B61}′, or two R^{B6}′ on the same atom are taken together to form ═O, ═C(-L^{B6}′-R^{B61})₂, ═N-L^{B6}′-R^{B61}′, or optionally substituted ═CH₂ or ═NH, wherein each R^{B61}′ is independently R′, and W^{X6}′ is O, S or Se;
X⁷′ is —C(R^{B7}′)═, —C(OR^{B7}′)═, —C(R^{B7}′)₂—, —C(W^{X7}′)—, —C(—N(R^{B7}′)₂)═, —N(R^{B7}′), —N═ or optionally substituted —NH—, —CH₂— or —CH═, wherein each R^{B7}′ is independently halogen, —CN, —NO₂, or -L^{B7}′-R^{B71}′, or two R^{B7}′ on the same atom are taken together to form ═O, ═C(-L^{B7}′-R^{B71}′)₂, ═N-L^{B7}′-R^{B71}′, or optionally substituted ═CH₂ or ═NH, wherein each R^{B71}′ is independently R′, and wherein W^{X7}′is O, S, or Se;
each of X⁸′ and X⁹′ is independently C or N;
each of L^{B2}′, L^{B3}′, L^{B4}′, L^{B5}′, L^{B6}′ and L^{B7}′ is independently L^{B}; and
each L^{B} is independently a covalent bond, or an optionally substituted bivalent C₁₋₁₀ saturated or partially unsaturated chain having 0-6 heteroatoms, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted, 3-20 membered, monocyclic, bicyclic or polycyclic ring having 0-10 heteroatoms;
each R′ is independently —R, —C(O)R, —C(O)OR, —C(O)N(R)₂, or —SO₂R; and
each R is independently —H, or an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

9. The oligonucleotide of any one of the preceding claims, wherein the length of the first domain is about 10-30 nucleobases.

10. The oligonucleotide of any one of the preceding claims, wherein the length of the second domain is about 10-30 nucleobases.

11. The oligonucleotide of any one of the preceding claims, wherein the length of the gap is about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 nucleobases.

12. The oligonucleotide of any one of the preceding claims, wherein the first domain is connected to the second domain through a linker, optionally wherein the linker is or comprises an oligonucleotide (“linker oligonucleotide”).

13. The oligonucleotide of any one of the preceding claims, wherein the complementarity of the linker oligonucleotide to the gap in the target nucleic acid is no more than about 30%, 25%, 20%, 10%, or 5%.

14. The oligonucleotide of any one of the preceding claims, wherein the linker comprises polyvinylether, polyethylene, polypropylene, polyethylene glycol (PEG), polypropylene glycol (PEG), polyvinyl alcohol (PVA), polyglycolide (PGA), polylactide (PLA), polycaprolactone (PCL), or copolymers thereof.

15. The oligonucleotide of any one of the preceding claims, wherein the target nucleic acid is mRNA.

16. The oligonucleotide of any one of the preceding claims, wherein when the oligonucleotide is contacted with a target nucleic acid comprising a target adenosine in a system, a target adenosine in the target nucleic acid is modified.

17. The oligonucleotide of any one of the preceding claims, wherein the target adenosine is a mutation from guanine.

18. The oligonucleotide of any one of the preceding claims, wherein the oligonucleotide has a length of about 10-200 (e.g., about 10-20, 10-30, 10-40, 10-50, 10-60, 10-70, 10-80, 10-90, 10-100, 10-120, 10-150, 20-30, 20-40, 20-50, 20-60, 20-70, 20-80, 20-90, 20-100, 20-120, 20-150, 20-200, 25-30, 25-40, 25-50, 25-60, 25-70, 25-80, 25-90, 25-100, 25-120, 25-150, 25-200, 30-40, 30-50, 30-60, 30-70, 30-80, 30-90, 30-100, 30-120, 30-150, 30-200, 10, 20, 25, 26, 27, 28, 29, 30, 31, 32, 33, 34, 35, 36, 37, 38, 39, 40, 45, 50, 60, etc.) nucleobases.

19. The oligonucleotide of any one of the preceding claims, wherein the oligonucleotide has a length of about 30-40 nucleobases.

20. The oligonucleotide of any one of the preceding claims, wherein the first domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.

21. The oligonucleotide of any one of the preceding claims, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first domain is chirally controlled.

22. The oligonucleotide of any one of the preceding claims, wherein the second domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.

23. The oligonucleotide of any one of the preceding claims, wherein the second domain comprises one or more (e.g., 1-10, 1, 2, 3, 4, 5, 6, 7, 8, 9, or 10, etc.) wobble pairs when the oligonucleotide is aligned with a target nucleic acid for complementarity.

24. The oligonucleotide of any one of the preceding claims, wherein the second domain comprise a nucleoside opposite to a target adenosine when the oligonucleotide is aligned with a target nucleic acid for complementarity.

25. The oligonucleotide of claim 95, wherein the first and second domain each independently comprise a nucleoside opposite to a target adenosine when the oligonucleotide is aligned with a target nucleic acid for complementarity.

26. The oligonucleotide of claim 25, wherein the first and second domain each independently comprise a nucleobase opposite to a target adenosine, wherein each opposite nucleobase comprises nucleobase BA, wherein BA is or comprises Ring BA or a tautomer thereof, wherein Ring BA has the structure of formula BA-III-e: 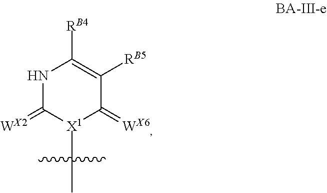
wherein:
X¹ is —N(−)- or -C(−)=;
each of W^{x2} and W^{X6} is independently O, S or Se;
R^{B4} is halogen, —CN, —NO₂, or -L^{B4}-R^{B41}, wherein R^{B41} is R′;
R^{B5} is halogen, —CN, —NO₂, or -L^{B5}-R^{B51}, wherein R^{B51} is —R′, —N(R′)₂, —OR′, or —SR′,
each of L^{B4} and L^{B5} is independently L^{B};
each L^{B} is independently a covalent bond, or an optionally substituted bivalent C₁₋₁₀ saturated or partially unsaturated chain having 0-6 heteroatoms, wherein one or more methylene unit is optionally and independently replaced with —Cy—, —O—, -S-, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted, 3-20 membered, monocyclic, bicyclic or polycyclic ring having 0-10 heteroatoms;
each R′ is independently —R, —C(O)R, —C(O)OR, —C(O)N(R)₂, or —SO₂R; and
each R is independently —H, or an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

27. The oligonucleotide of any one of claims 1-3, wherein the first and second domain each independently comprise a nucleobase opposite to a target adenosine, wherein each opposite nucleobase comprises nucleobase BA, wherein BA is or comprises Ring BA or a tautomer thereof, wherein Ring BA has the structure of formula BA-VI: 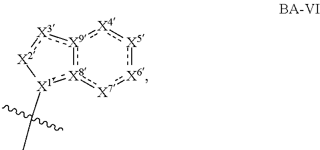
wherein:
each [symbol] is independent a single or double bond;
X¹′ is —N(−)- or —C(−)=;
X²′ is —C(W^{X2}′)—, —C(R^{B2}′)═, C(OR^{B2}′)═, —N═, or optionally substituted —CH═ or —CH₂—, wherein R^{B2}′ is halogen, —CN, —NO₂, or -L^{B2}′-R′, and W^{X2}′ is O, S or Se;
X³′ is —N(R^{B3}′)—, —N═, —C(R^{B3}′)═ or optionally substituted —NH— or —CH═, wherein R^{B3} is halogen, —CN, —NO₂, or -L^{B3}′-R′;
X⁴′ is —C(R^{B4}′)═, —C(OR^{B4}′)═, —C(—N(R^{B4}′)₂)═, —C(R^{B4}′)₂—, —C(W^{X4}′)—, —C(═NR^{B4}′)—, —N(R^{B4}′)—, —N═, or optionally substituted —CH═, —NH— or —CH₂—, wherein each R^{B4}′ is independently halogen, —CN, —NO₂, or -L^{B4}′_R^{B41}′, or two R^{B4}′ on the same atom are taken together to form ═O, =C(-L^{B4}′-R^{B41}′)₂, ═N-L^{B4}′- R^{B41}′, or optionally substituted ═CH₂ or ═NH, wherein each R^{B41}′ is independently —R′, and W^{X4}′ is O, S or Se;
X⁵′ is —C(R^{B5}′)₂—, —N(R^{B5}′), —C(R^{B5}′)═, —C(W^{X5}′)—, —N═, or optionally substituted —NH—, —CH₂—, or —CH═, wherein each R^{B5}′ is independently halogen, —CN, —NO₂, or -L^{B5}′R^{B51}′, wherein R^{B51}′ is —R′, —N(R′)₂, —OR′, or —SR′, and W^{X5}′ is O, S, or Se;
X^{6′} is —C(R^{B6}′)═, C(OR^{B6}′)═, —C(R^{B6}′)₂—, —C(W^{X6}′)—, —C(—N(R^{B6}′)₂)═, —N═ or optionally substituted —NH—, —CH₂— or —CH═, wherein each R^{B6}′ is independently halogen, —CN, —NO₂, or -L^{B6}′-R^{B61}′, or two R^{B6}′ on the same atom are taken together to form ═O, ═C(-L^{B6}′-R^{B61}′)₂, ═N-L^{B6}′-R^{B61}′ or optionally substituted ═CH₂ or ═NH, wherein each R^{B61}′ is independently R′, and W^{X6}′ is O, S or Se;
X⁷′ is —C(R^{B7}′)═, —C(OR^{B7}′)═, —C(R^{B7}′)₂-, —C(W^{X7}′), —C(—N(R^{B7}′)₂)═, —N(R^{B7}′)-, —N═ or optionally substituted —NH—, —CH₂— or —CH═, wherein each R^{B7}′ is independently halogen, —CN, —NO₂, or -L^{B7}′-R^{B71}′, or two R^{B7}′ on the same atom are taken together to form ═O, ═C(-L^{B7}′-R^{B71}′)₂, =N-L^{B7}-R^{B71}′, or optionally substituted ═CH₂ or ═NH, wherein each R^{B71}′ is independently R′, and wherein W^{X7}′ is O, S, or Se;
each of X⁸′ and X⁹′ is independently C or N;
each of L^{B2}′, L^{B3}′, L^{B4}′, L^{B5}′, L^{B6}′ and L^{B7}′ is independently L^{B}; and
each L^{B} is independently a covalent bond, or an optionally substituted bivalent C₁₋₁₀ saturated or partially unsaturated chain having 0-6 heteroatoms, wherein one or more methylene unit is optionally and independently replaced with —Cy—, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted, 3-20 membered, monocyclic, bicyclic or polycyclic ring having 0-10 heteroatoms;
each R′ is independently —R, —C(O)R, —C(O)OR, —C(O)N(R)₂, or —SO₂R; and
each R is independently —H, or an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

28. The oligonucleotide of any one of claims 1-26, wherein the sugar of the nucleoside comprising the opposite nucleobase comprises a 2′-OR modification, wherein R is optionally substituted C₁₋₆ aliphatic.

29. The oligonucleotide of any one of claims 1-26, wherein the sugar of the nucleoside comprising the opposite nucleobase comprises a natural DNA sugar.

30. A phosphoramidite, wherein the nucleobase of the phosphoramidite is a nucleobase of any one of claims 1-28 or a tautomer thereof, wherein the nucleobase or tautomer thereof is optionally substituted or protected.

31. The phosphoramidite of claim 30, wherein the phosphoramidite has the structure of 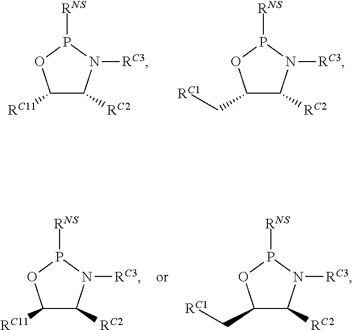
or a salt thereof.

32. A method for preparing an oligonucleotide or composition, comprising coupling a —OH group of an oligonucleotide or a nucleoside with a phosphoramidite of any one of claims 30-31.

33. A method for deaminating a target adenosine in a target nucleic acid, comprising contacting the target nucleic acid with an oligonucleotide or composition of any one of the preceding claims, wherein the oligonucleotide targets the target adenosine.

34. A method for producing, or restoring or increasing level of a particular nucleic acid or a product thereof, comprising contacting a target nucleic acid with an oligonucleotide or composition of any one of the preceding claims, wherein the target nucleic acid comprises a target adenosine, and the particular nucleic acid differs from the target nucleic acid in that the particular nucleic acid has an I or G instead of the target adenosine.

35. A method, comprising:
contacting an oligonucleotide or composition of any one of the preceding claims with a sample comprising a target nucleic acid and an adenosine deaminase, wherein:
the base sequence of the oligonucleotide or oligonucleotides in the oligonucleotide composition is substantially complementary to that of the target nucleic acid; and
the target nucleic acid comprises a target adenosine;
wherein the target adenosine is modified.

36. A method, comprising
obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and
wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;
wherein:
oligonucleotides of the first plurality comprise one or more chirally controlled chiral internucleotidic linkages; and
oligonucleotides of the reference plurality comprise no chirally controlled chiral internucleotidic linkages (a reference oligonucleotide composition is a “stereorandom composition).

37. A method for preventing a condition, disorder or disease associated with a G to A mutation, comprising administering or delivering to a subject susceptible thereto an effective amount of an oligonucleotide or composition of any one of the preceding claims, wherein the oligonucleotide targets the G to A mutation for editing.

38. A method for treating a condition, disorder or disease associated with a G to A mutation, comprising administering or delivering to a subject suffering therefrom an effective amount of an oligonucleotide or composition of any one of the preceding claims, wherein the oligonucleotide targets the G to A mutation for editing.

39. An oligonucleotide, composition, phosphoramidite, use, or method described in the specification or any one of Example Embodiments 1-2349.
